SEQUENCE LISTING (13 sequences)

SEQ ID NO: 1 - protein, 183 aa, synthetic construct
GLNDIFEAQKIEWHETEYKLVVVGACGVGKSALTIQLIQNHFVDEYDPTIEDSYRKQVVIDGETSLLDILDTAGQEEYSAMRDQYMRTGEGFLLVFAINNTKSFEDIHHYREQIKRVKDSEDVPMVLVGNKSDLPSRTVDTKQAQDLARSYGIPFIETSAKTRQGVDDAFYTLVREIRKHKEK

SEQ ID NO: 2 - protein, 183 aa, synthetic construct
GLNDIFEAQKIEWHETEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 3 - protein, 183 aa, synthetic construct
GLNDIFEAQKIEWHETEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 4 - protein, 183 aa, synthetic construct
GLNDIFEAQKIEWHETEYKLVVVGAGDVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 5 - protein, 172 aa, synthetic construct
GGGGSHMTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQH

SEQ ID NO: 6 - protein, 176 aa, synthetic construct
GGGGMTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLN

SEQ ID NO: 7 - protein, 524 aa, synthetic construct
MGSSHHHHHHSGENLYFQGSSGLNDIFEAQKIEWHESSEEQMRLPSADVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEITGEIQQYQNQPYCLRVESDIKRFFENLNPMGNSMEKEFTDYLFNKSLEIEPRNPKPLPRFPKKYSYPLKSPGVRPSNPRPGT

SEQ ID NO: 8 - protein, 172 aa, synthetic construct
GGGGTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 9 - protein, 188 aa, synthetic construct
MASSHHHHHHSSENLYFQGMTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 10 - protein, 188 aa, synthetic construct
MASSHHHHHHSSENLYFQGMTEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 11 - protein, 210 aa, synthetic construct
MGSSHHHHHHSSGLVPRGSHMASMTGGQQMGRGSENLYFQGMTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 12 - protein, 188 aa, synthetic construct
MASSHHHHHHSSENLYFQGMTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 13 - protein, 487 aa, synthetic construct
GEEQMRLPSADVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEITGEIQQYQNQPYCLRVESDIKRFFENLNPMGNSMEKEFTDYLFNKSLEIEPRNPKPLPRFPKKYSYPLKSPGVRPSNPRPGT

(19) United States
(12) Patent Application Publication
(10) Pub. No.: US 2026/0176289 A1
(43) Pub. Date: Jun. 25, 2026

(54) SMALL MOLECULE INHIBITORS OF KRAS PROTEINS

(71) Applicant: Merck Sharp & Dohme LLC, Rahway, NJ (US)

(72) Inventors: Symon Gathiaka, Waltham, MA (US); Shuhei Kawamura, Cambridge, MA (US); Matthew J. Mitcheltree, Jamaica Plain, MA (US); Anandan Palani, Needham, MA (US); Juan del Pozo, Brookline, MA (US); Uma Swaminathan, Auburndale, MA (US); Hua Zhou, Acton, MA (US); Yu Kobayakawa, Ibaraki (JP); Satoru Iguchi, Ibaraki (JP); Hitomi Kondo, Ibaraki (JP); Keiichi Sumiyama, Ibaraki (JP); Patrick Schöpf, London (GB); Steven Howard, Cambridge (GB); George Madalin Giambasu, Natick, MA (US); Melanie A. S. Blackburn, Watertown, MA (US); Solomon Kattar, Wakefield, MA (US); Igri Kolaj, Malden, MA (US); Bing Li, Towaco, NJ (US); Michael Ryan, Brookline, MA (US); Emily Ruan, Boston, MA (US); Tobias Sandmeier, Brighton, MA (US); David L. Sloman, Newton, MA (US); Tomohiro Yamamoto, Ibaraki (JP); Makoto Kitade, Ibaraki (JP); Junpei Koyama, Ibaraki (JP); Tsuyoshi Oshima, Ibaraki (JP); Kei Akemoto, Ibaraki (JP); Toshihiro Sakamoto, Ibaraki (JP); Hiroki Asakura, Ibaraki (JP); Risako Miura, Ibaraki (JP)

(21) Appl. No.: 19/128,682

(22) PCT Filed: Nov. 10, 2023
(86) PCT No.: PCT/US2023/079393

§ 371 Date: May 9, 2025

Related U.S. Application Data
(60) Provisional application No. 63/383,342, filed on Nov. 11, 2022.

(51) Int. Cl. C07D 519/00; A61K 31/519; A61K 31/5377; A61K 31/55; A61K 31/553; A61K 45/06; C07B 59/00; C07D 471/14; C07D 487/14; C07D 491/147; C07D 495/14

(52) U.S. Cl. CPC C07D 519/00; A61K 31/519; A61K 31/5377; A61K 31/55; A61K 31/553; A61K 45/06; C07B 59/002; C07D 471/14; C07D 487/14; C07D 491/147; C07D 495/14

(57) ABSTRACT

Compounds of Formula (I) or their pharmaceutically acceptable salts can inhibit the G12C, G12D, G12V, and/or G13D mutants of Kirsten rat sarcoma (KRAS) protein and are expected to have utility as therapeutic agents, for example, for treating cancer. The disclosure also provides pharmaceutical compositions which comprise compounds of Formula (I) or pharmaceutically acceptable salts thereof. The disclosure also relates to methods for use of the compounds or their pharmaceutically acceptable salts in the therapy and prophylaxis of cancer and for preparing pharmaceuticals for this purpose.

### FIELD OF THE INVENTION

The present disclosure relates to small molecule inhibitors of KRAS that inhibit, for example, the G12C mutant, G12D mutant, G12V mutant, G13D mutant, and the wild-type (WT) of Kirsten rat sarcoma (KRAS) protein and relates to a pharmaceutical composition comprising a compound of Formula (I) as well as methods of using such a compound for treatment of diseases, including cancers.

### BACKGROUND

RAS, which is a small monomeric GTP-binding protein having a molecular weight of about 21 kDa, acts as a molecular on/off switch. RAS can bind to GTP by binding to proteins of a guanine nucleotide exchange factor (GEF) (e.g., SOS1), which forces the release of a bound nucleotide, and releases GDP. When RAS binds to GTP, it becomes activated (turned on) and recruits and activates proteins necessary for the propagation of other receptors' signals, such as c-Raf and PI 3-kinase. RAS also possesses enzymatic activity with which it cleaves the terminal phosphate of the GTP nucleotide and converts the nucleotide into GDP. The rate of conversion is usually slow, but can be dramatically sped up by a protein of the GTPase-activating protein (GAP) class, such as RasGAP. When GTP is converted into GDP, RAS is deactivated (turned off).

The commonly known members of the RAS subfamily include HRAS, KRAS, and NRAS. Of these, mutations of KRAS are observed in many malignant tumors: in 86% of pancreatic ductal adenocarcinoma (PDAC), in 41% of colorectal cancers (CRC), and in 32% of lung adenocarcinoma (LUAD; a subtype of non-small-cell lung cancer (NSCLC)). The mutations often occur in the glycine residue at position 12 of KRAS (“G12”); the mutation at G12 dominates 91% (PDAC), 68% (CRC) and 85% (LUAD) of the total KRAS mutations, respectively. The distributions of amino acid substitutions at G12 vary among each tissue type. The most prevalent mutation in LUAD is the mutation into cysteine (“G12C”) (46%), while the predominant mutation in PDAC (45%) and CRC (45%) is the mutation into aspartic acid (“G12D”). The mutation at G12 into valine (“G12V”) is observed in a significant portion of G12 mutations in all of PDAC (35%), CRC (30%) and LUAD (23%). (Nature Reviews Drug Discovery, 19, 533-552, 2020).

Intense efforts in developing KRAS-G12C inhibitors are underway. Several covalent inhibitors which focus on the cysteine residue have been reported, and some of them have been subjected to clinical studies, such as AMG510 (NCT03600883), MRTX849 (NCT03785249) and JNJ-74699157 (NCT04006301). However, the KRAS-G12C mutation only accounts for a fraction of all KRAS mutations and is primarily found in LUAD. To effectively inhibit the other commonly-occurring KRAS mutated proteins, such as KRAS-G12D and KRAS-G12V, different approaches are needed as these mutants lack reactive cysteines in the active site (Nature Reviews Drug Discovery, 19, 533-552, 2020).

Studies have also indicated that gene amplification and high expression of WT KRAS in the absence of coding mutations can also occur in certain cancers. These amplifications were observed most frequently in esophageal, gastric and ovarian adenocarcinomas (Nature Medicine, 24, 968-977, 2018). Thus, effective inhibition of WT KRAS could provide a therapeutic benefit to patients suffering from such cancers.

### SUMMARY OF THE DISCLOSURE

The present disclosure provides small molecule inhibitors which modulate mutant and WT KRAS proteins and may be valuable pharmaceutically active compounds for the treatment of cancer. In some embodiments the disclosed compounds selectively inhibit the KRAS-G12C, KRAS-G12D and/or KRAS-G12V proteins. The compounds of Formula (I):

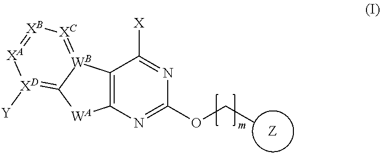

and their pharmaceutically acceptable salts, can modulate the activity of KRAS and thereby affect the signaling pathway which regulates cell growth, differentiation, and proliferation associated with oncological disorders. In certain embodiments, the compounds of Formula (I) can inhibit the KRAS-G12C, KRAS-G12D, KRAS-G12V, KRAS-G13D, and/or WT KRAS proteins. The disclosure furthermore provides processes for preparing compounds of Formula (I), methods for using such compounds to treat oncological disorders, and pharmaceutical compositions which comprise compounds of Formula (I).

### DETAILED DESCRIPTION OF THE INVENTION

### Compounds of the Disclosure

In one embodiment, the present disclosure provides a compound having structural Formula (I), or a pharmaceutically acceptable salt thereof, as shown above, wherein;
X is:

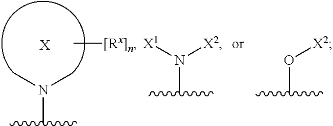

Ring X is selected from the group consisting of:
   (i) a 5- to 9-membered monocyclic or fused bicyclic or bridged bicyclic heterocycloalkyl, wherein the 5- to 9-membered monocyclic or fused bicyclic or bridged bicyclic heterocycloalkyl is saturated and contains 0 to 2 heteroatom groups selected from the group consisting of N, S, S(O), S(O)₂ and O, in addition to the illustrated N atom; and
   (ii) an 8- to 12-membered spiroheterocycloalkyl, wherein the 8- to 12-membered spiroheterocycloalkyl is saturated and contains 0 to 2 heteroatom groups selected from the group consisting of N, S, S(O), S(O)₂ and O, in addition to the illustrated N atom;
each R^{X} is independently selected from the group consisting of fluoro, cyano, hydroxy, oxo, C₁-C₆ alkyl, C₁-C₆ fluoroalkyl, C₁-C₆ alkoxy, C₁-C₆ fluoroalkoxy, C₁-C₆ cyanoalkyl, and C₁-C₆ hydroxyalkyl;
X¹ is selected from the group consisting of H, C₁-C₆ alkyl, C₁-C₆ fluoroalkyl, C₁-C₆ fluoroalkoxy, C₃-C₆ cycloalkyl, and C₃-C₆ fluorocycloalkyl;
X² is H or —(CR^{a}₂)ₚ—C^{X}, wherein C^{X} is:
   (i) a 3- to 10-membered monocyclic or fused bicyclic or bridged bicyclic cycloalkyl or an 8- to 10-membered spirocycloalkyl;
   (ii) a 3- to 10-membered monocyclic or fused bicyclic or bridged bicyclic heterocycloalkyl or an 8- to 10-membered spiroheterocycloalkyl, wherein the 3- to 10-membered monocyclic or fused bicyclic or bridged heterocycloalkyl or the 8- to 10-membered spiroheterocycloalkyl is saturated and contains 1 to 3 heteroatom groups independently selected from the group consisting of N, S, S(O), S(O)₂ and O;
      wherein C^{X} is unsubstituted or substituted by 1 to 2 RCA substituents independently selected from the group consisting of fluoro, hydroxy, oxo, cyano, C₁-C₆ alkyl, C₁-C₆ acyl, C₁-C₆ fluoroalkyl, C₁-C₆ alkoxy, C₁-C₆ fluoroalkoxy, C₁-C₆ hydroxyalkyl, C₁-C₆ cyanoalkyl, C₃-C₆ cycloalkyl, and C₃-C₆ fluorocycloalkyl;
each R^{a} is independently selected from the group consisting of hydrogen, halo, cyano, hydroxy, C₁-C₆ alkyl, C₁-C₆ fluoroalkyl, C₁-C₆ alkoxy, C₁-C₆ fluoroalkoxy, C₁-C₆ cyanoalkyl, C₁-C₆ fluorocyanoalkyl, C₃-C₆ cycloalkyl, and C₃-C₆ fluorocycloalkyl;
X^{A}, X^{B}, and X^{C} are independently selected from the group consisting of N, C(H), and C(R¹);
X^{D} is selected from the group consisting of N and C;
each R¹ is independently selected from the group consisting of C₁-C₃ alkyl, C₁-C₃ fluoroalkyl, C₁-C₃ alkoxy, C₁-C₃ fluoroalkoxy, halo, hydroxy, oxo, cyano, C₁-C₃ alkylamino, C₁-C₃ dialkylamino, and C^{B};
   wherein C^{B} is selected from the group consisting of:
   (i) a monocyclic, saturated heterocycloalkyl containing 1 to 3 heteroatoms independently selected from the group consisting of N, S, and O;
   (ii) a monocyclic 5- to 6-membered heteroaryl containing 1 to 3 heteroatoms independently selected from the group consisting of N, S, and O;
      wherein C^{B} is unsubstituted or substituted by 1 to 2 R^{C}^{^{B}} substituents independently selected from the group consisting of halo, hydroxy, oxo, cyano, C₁-C₃ alkyl, C₁-C₃ fluoroalkyl, C₁-C₃ alkoxy, C₁-C₃ fluoroalkoxy, C₃-C₆ cycloalkyl, and C₃-C₆ fluorocycloalkyl;
W^{A} is selected from the group consisting of N(R^{W1}), C(R^{W2})₂, O, S, Se, and C(R^{W3});
R^{W1} is selected from the group consisting of C₁-C₃ alkyl, C₁-C₃ fluoroalkyl, C₃-C₆ cycloalkyl, and C₃-C₆ fluorocycloalkyl;
each R^{W2} is independently selected from the group consisting of fluoro, C₁-C₃ alkyl, C₁-C₃ fluoroalkyl, and hydroxy;
   or alternatively, the two R^{W2}, together with the carbon atom to which they are attached form a C₃-C₆ cycloalkyl or C₃-C₆ fluorocycloalkyl; R^{W3} is selected from the group consisting of H, halo, C₁-C₃ alkyl, C₁-C₃ alkoxy, C₁-C₃ fluoroalkyl, C₁-C₃ fluoroalkoxy, C₃-C₆ cycloalkyl, and C₃-C₆ fluorocycloalkyl;
W^{B} is C or N, wherein when:
   W^{B} is C, then W^{A} is N(R^{W1}), C(R^{W2})₂, O, or S; and
   W^{B} is N, then W^{A} is C(R^{W3});
Y is selected from the group consisting of C₁-C₆ alkyl, C₁-C₆ fluoroalkyl, and C^{Y};
C^{Y} is:
   (i) an aryl selected from the group consisting of phenyl and naphthyl:
   (ii) an indanyl or fused indanyl group of the formula:

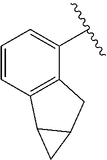 [SMILES: *c1cccc2c1CC1CC21]

(iii) a 5- to 6-membered monocyclic heteroaryl, wherein the 5- to 6-membered monocyclic heteroaryl contains 1 to 3 heteroatoms independently selected from the group consisting of N, S, and O; or
(iv) a 9- to 10-membered fused, bridged or spiro-bicyclic heteroaryl, wherein the 9- to 10-membered fused, bridged or spiro-bicyclic heteroaryl contains 1 to 3 heteroatoms independently selected from the group consisting of N, O, and S; or
(v) a 12- to 17-membered fused, bridged or spiro-tri- or tetracyclic heterocycloalkyl, where at least 2 of the rings of the 12- to 17-membered fused, bridged or spiro-tri- or tetracyclic heterocycloalkyl are aromatic, the third ring is partially unsaturated or aromatic, and the fourth ring, if present, is saturated, wherein the 12- to 17-membered fused, bridged or spiro-tri- or tetracyclic heterocycloalkyl contains 1 to 4 heteroatoms independently selected from the group consisting of N, S, and O;
wherein C^{Y} is unsubstituted or substituted by 1 to 5 RY substituents independently selected from the group consisting of halo, hydroxy, oxo, cyano, C₁-C₆ alkyl, C₂-C₆ alkynyl, C₂-C₇ alkenyl, C₁-C₆ fluoroalkyl, C₁-C₆ alkoxy, C₁-C₆ haloalkoxy, C₂-C₆ fluoroalkynyl, C₂-C₇ fluoroalkenyl, C₁-C₃ fluoroalkenylenyl, C₁-C₆ alkylthio, C₁-C₆ fluoroalkylthio, amino, C₁-C₆ alkylamino, C₁-C₆ dialkylamino, tri (C₁-C₆ alkyl) silyl, cyano, C₁-C₆ cyanoalkyl, C₁-C₆ fluorocyanoalkyl, C₁-C₃ alkoxy C₁-C₃ alkyl, C₁-C₆ alkoxycarbonyl, C₁-C₆ acyl, methylenyl, and R^{ya};
   wherein R^{ya} is:
      (a) a 3- to 9-membered monocyclic or fused bicyclic or bridged bicyclic cycloalkyl;
      (b) a 3- to 9-membered monocyclic or fused bicyclic or bridged bicyclic cycloalkenyl;
      (c) a 5- to 6-membered heteroaryl containing 1 to 3 heteroatoms independently selected from the group consisting of N, S, and O; or
      (d) a 4- to 7-membered saturated or partially saturated heterocycloalkyl containing 1 to 2 heteroatoms independently selected from the group of N, S, and O;
      wherein R^{ya} is unsubstituted or substituted by 1 to 2 substituents independently selected from the group consisting of halo, hydroxy, cyano, C₁-C₃ alkyl, C₁-C₃ fluoroalkyl, C₃-C₆ cycloalkyl, C₃-C₆ fluorocycloalkyl, C₁-C₃ alkoxy, C₁-C₃ fluoroalkoxy, and C₁-C₃ cyanoalkyl;

Ring Z is selected from the group consisting of:
(i) a 3- to 10-membered monocyclic, fused, bridged or spiro-bicyclic, or fused, bridged or spiro-tricyclic heterocycloalkyl, wherein the 3- to 10-membered monocyclic, fused, bridged or spiro-bicyclic, or fused, bridged or spiro-tricyclic heterocycloalkyl is saturated or partially unsaturated and contains 1 to 3 heteroatoms independently selected from the group consisting of N, S, and O;
(ii) a 3- to 10-membered monocyclic, fused, bridged or spiro-bicyclic, or fused, bridged or spiro-tricyclic cycloalkyl, wherein the 3- to 10-membered monocyclic, fused, bridged or spiro-bicyclic, or fused, bridged or spiro-tricyclic cycloalkyl is saturated or partially unsaturated; and
   (iii)

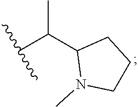 [SMILES: *C(C)C1CCCN1C]

wherein ring Z is unsubstituted or independently substituted with 1 to 4 substituents R^{ZC} selected from the group consisting of halo, hydroxy, oxo, cyano, C₁-C₃ alkyl, C₁-C₃ fluoroalkyl, C₁-C₃ hydroxyalkyl, C₁-C₃ fluoroalkenylenyl, C₁-C₃ hydroxyfluoroalkyl, C₁-C₃ alkoxy, C₁-C₃ fluoroalkoxy, C₁-C₃ cyanoalkyl, C₃-C₆ cycloalkyl, C₃-C₆ fluorocycloalkyl, C₃-C₆ hydroxycycloalkyl, C₃-C₆ hydroxyfluorocycloalkyl, C₂-C₄ fluoroalkenyl, C₁-C₃ alkylamino, C₁-C₃ dialkylamino, methylene (C₁-C₃ alkyl)amino, C₁-C₃ alkylenedi(C₁-C₃ alkyl)amino, and methylene (C₁-C₃ alkyl) (C₁-C₃ alkyl) carbamate;
wherein ring Z is optionally substituted with 1 P or —CH₂P, wherein P is a 4- to 10-membered monocyclic, fused, bridged or spiro-bicyclic, or fused, bridged or spiro-tricyclic heterocycloalkyl, wherein the 4- to 10-membered monocyclic, fused, bridged or spiro-bicyclic, or fused, bridged or spiro-tricyclic heterocycloalkyl is saturated or partially unsaturated and contains 1 to 2 heteroatoms selected from the group consisting of N, S, and O;
wherein P is unsubstituted or independently substituted with 1 to 4 R^{P} substituents selected from the group consisting of halo, hydroxy, oxo, cyano, C₁-C₃ alkyl, C₁-C₃ fluoroalkyl, C₁-C₃ hydroxyalkyl, C₁-C₃ hydroxyfluoroalkyl, C₁-C₃ alkoxy, C₁-C₃ fluoroalkoxy, C₁-C₃ cyanoalkyl, C₃-C₆ cycloalkyl, C₃-C₆ fluorocycloalkyl, C₃-C₆ hydroxycycloalkyl, C₁-C₃ alkoxy C₁-C₃ alkyl, C₃-C₆ hydroxyfluorocycloalkyl, carbamoyl, and —NHC(O)C₁-C₃alkyl;
subscript m is 0, 1, or 2;
subscript n is 0, 1, 2, or 3; and
subscript p is 0, 1, 2, or 3.

In another embodiment, the present disclosure provides a compound of Formula (I), or the pharmaceutically acceptable salt thereof, wherein X is

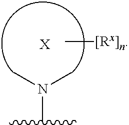

In another embodiment, the present disclosure provides a compound of Formula (I), or the pharmaceutically acceptable salt thereof, wherein X is

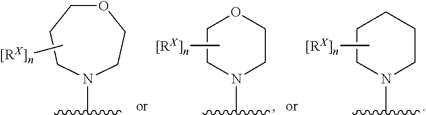

In another embodiment, the present disclosure provides a compound of Formula (I), or the pharmaceutically acceptable salt thereof, wherein X is

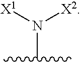

In another embodiment, the present disclosure provides a compound of Formula (I), or the pharmaceutically acceptable salt thereof, wherein X¹ is H and X² is —(CH₂)ₚ—C^{X}, wherein C^{X} is selected from the group consisting of:

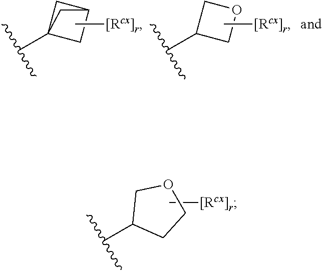

and
subscript r is 0, 1, or 2.

In another embodiment, the present disclosure provides a compound of Formula (I), or the pharmaceutically acceptable salt thereof, wherein X is

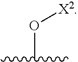

In another embodiment, the present disclosure provides a compound of Formula (I), or the pharmaceutically acceptable salt thereof, wherein W^{A} is N(R^{W1}).

In another embodiment, the present disclosure provides a compound of Formula (I), or the pharmaceutically acceptable salt thereof, wherein W^{A} is O.

In another embodiment, the present disclosure provides a compound of Formula (I), or the pharmaceutically acceptable salt thereof, wherein W^{A} is C(R^{W2})₂.

In another embodiment, the present disclosure provides a compound of Formula (I), or the pharmaceutically acceptable salt thereof, wherein W^{A} is C(R^{W3}).

In another embodiment, the present disclosure provides a compound of Formula (I), or the pharmaceutically acceptable salt thereof, wherein W^{A} is S.

In another embodiment, the present disclosure provides a compound of Formula (I), or the pharmaceutically acceptable salt thereof, wherein W^{A} is Se.

In another embodiment, the present disclosure provides a compound of Formula (I), or the pharmaceutically acceptable salt thereof, wherein Y is C^{Y}, and C^{Y} is unsubstituted or substituted phenyl, naphthyl, pyridyl, indazolyl, benzothienyl, benzoxazolyl, benzothiazolyl, or isoquinolinyl.

In another embodiment, the present disclosure provides a compound of Formula (I), or the pharmaceutically acceptable salt thereof, wherein C^{Y} is naphthyl and indazolyl, wherein C^{Y} is substituted by 1 to 3 RY substituents independently selected from the group consisting of halo, hydroxy, amino, C₁-C₃ alkyl, C₁-C₃ fluoroalkyl, C₃-C₆ cycloalkyl, C₃-C₆ fluorocycloalkyl, C₂-C₄ alkynyl, and cyano.

In another embodiment, the present disclosure provides a compound of Formula (I), or the pharmaceutically acceptable salt thereof, wherein C^{Y} is selected from the group consisting of:

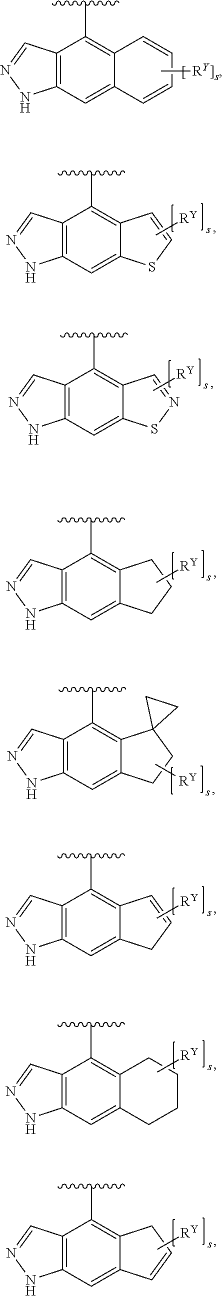

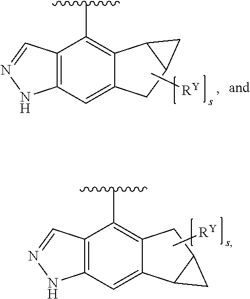

and subscript s is 0, 1, 2, 3, or 4.

In another embodiment, the present disclosure provides a compound of Formula (I), or the pharmaceutically acceptable salt thereof, wherein Y is C^{Y}, wherein C^{Y} is a group of the formula

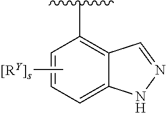

and subscript s is 0, 1, 2, or 3.

In another embodiment, the present disclosure provides a compound of Formula (I), or the pharmaceutically acceptable salt thereof, wherein Y is C^{Y}, wherein C^{Y} is selected from the group consisting of:

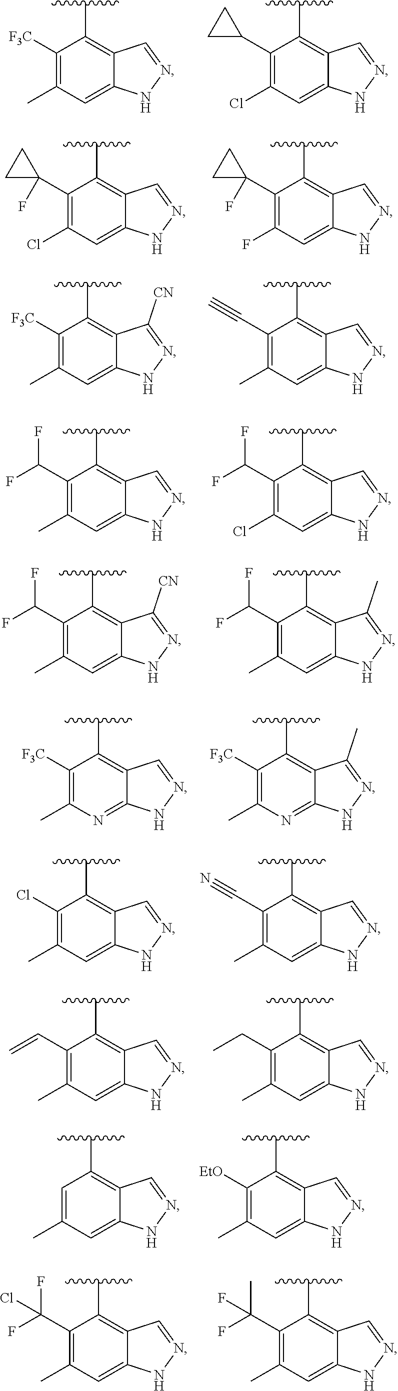

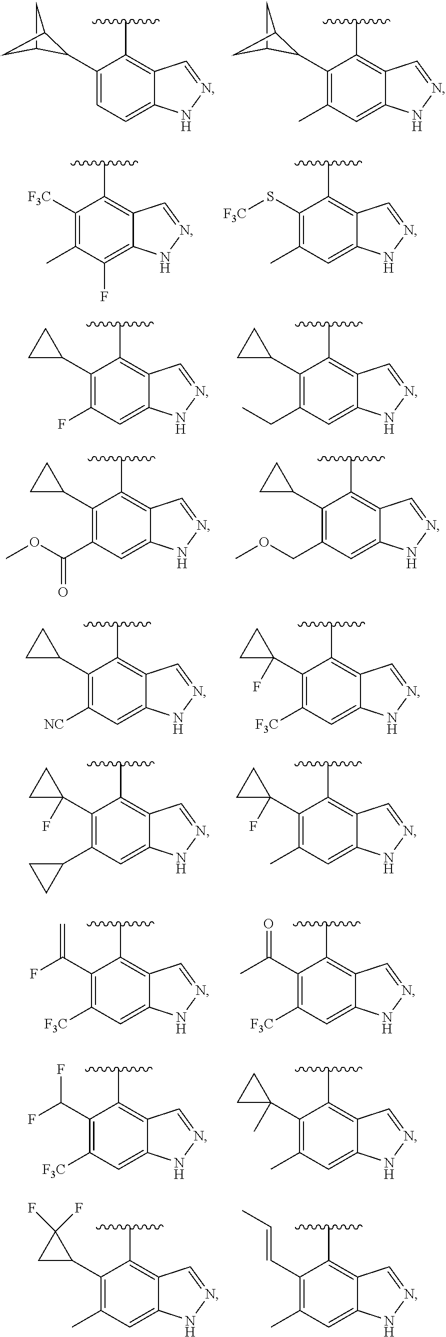

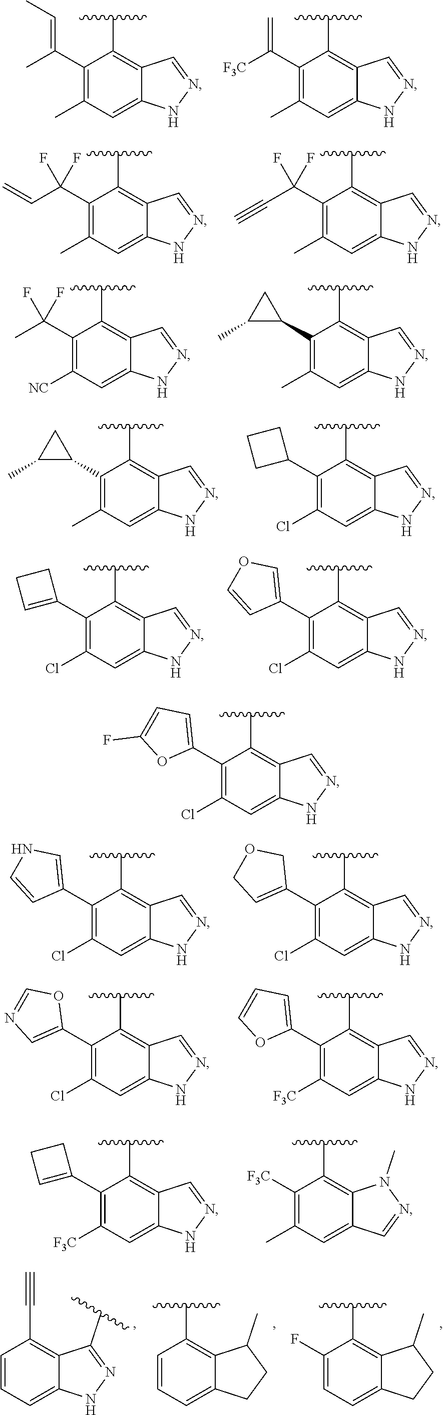

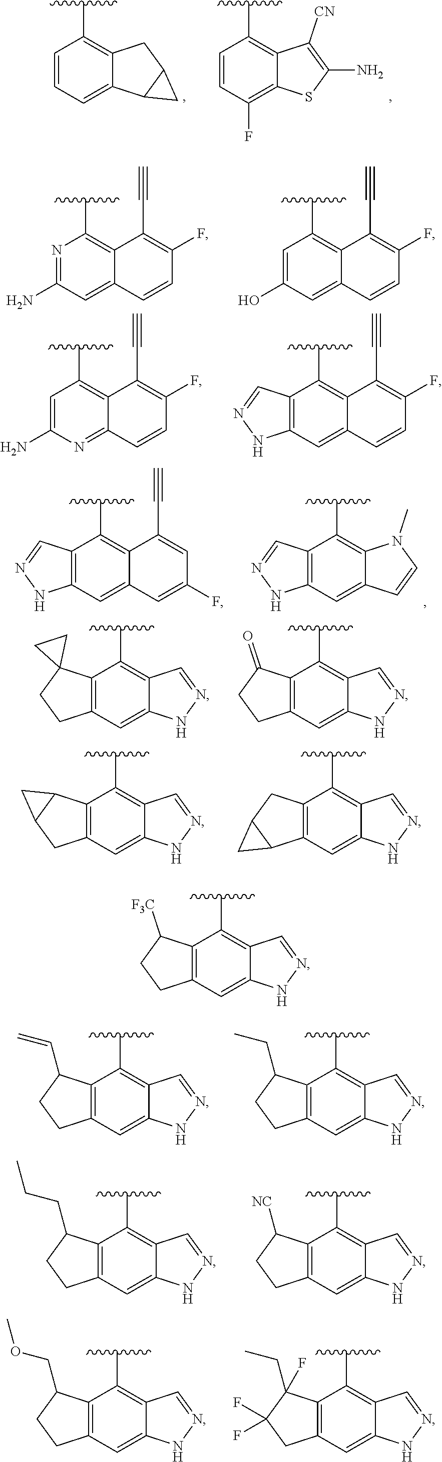

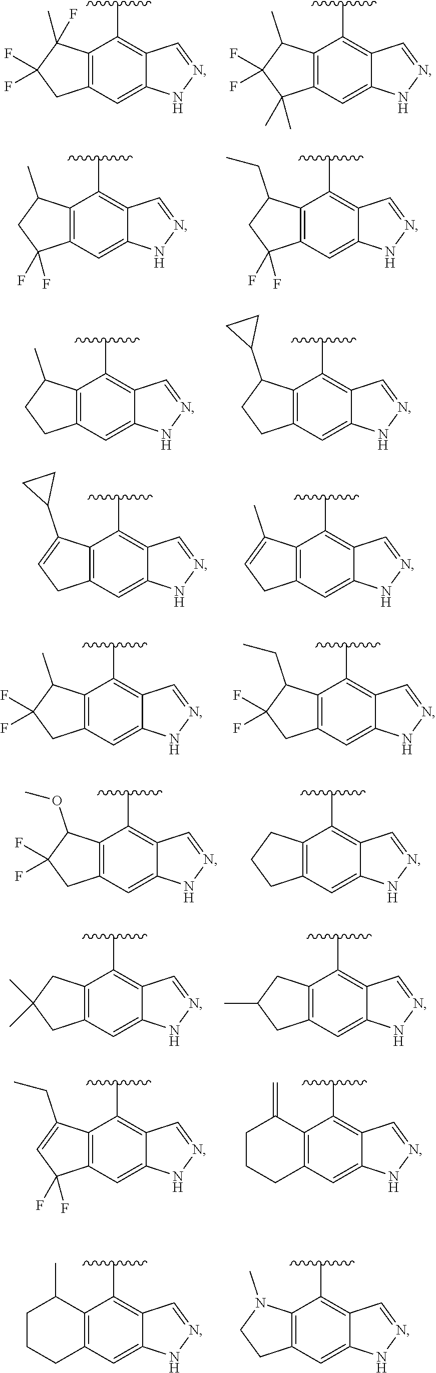

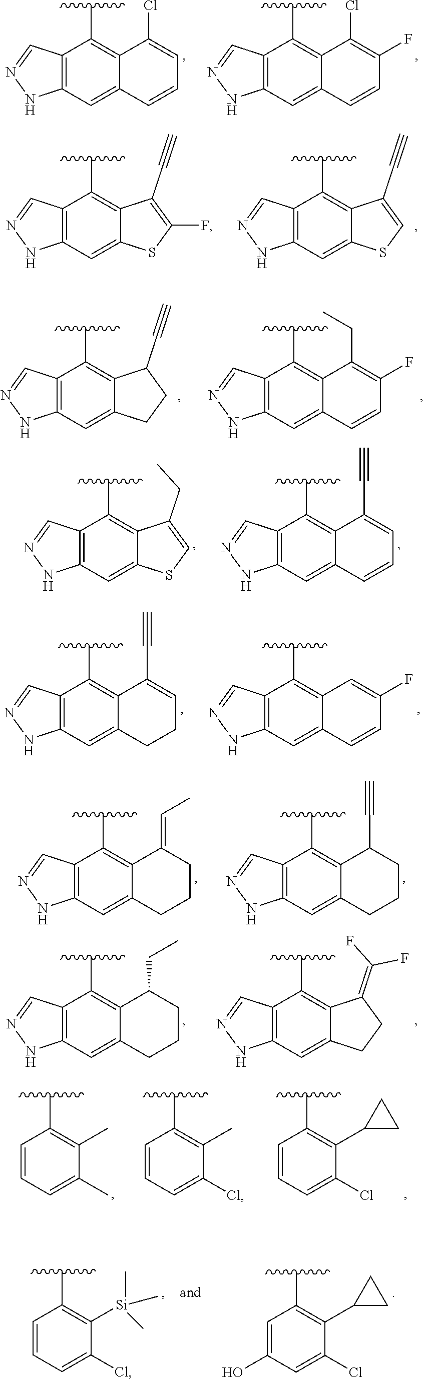

In another embodiment, the present disclosure provides a compound of Formula (I), or the pharmaceutically acceptable salt thereof, wherein
X^{A} is N,
X^{B} and X^{C} are independently selected from the group consisting of C(H) and (CR¹),
X^{D} is C, and
W^{B} is C.

In another embodiment, the present disclosure provides a compound of Formula (I), or the pharmaceutically acceptable salt thereof, wherein Ring Z is selected from the group consisting of:
(i) a 5- to 10-membered monocyclic, fused, bridged or spiro-bicyclic, or fused, bridged or spiro-tricyclic heterocycloalkyl, wherein the 5- to 10-membered monocyclic, fused, bridged or spiro-bicyclic, or fused, bridged or spiro-tricyclic heterocycloalkyl is saturated and contains 1 to 3 heteroatoms independently selected from the group consisting of N, S, and O, and wherein the 5- to 10-membered monocyclic, fused, bridged or spiro-bicyclic, or fused, bridged or spiro-tricyclic heterocycloalkyl is unsubstituted or substituted with 1 to 2 substituents R^{ZHC} selected from the group consisting of halo, hydroxy, C₁-C₃ alkyl, C₁-C₃ hydroxyalkyl, —C(H)(OH)CF₂H, —O—CH₂—O—(C₁-C₃ fluoroalkyl), C₂-C₄ fluoroalkenyl, and methylene (C₁-C₃ alkyl) (C₁-C₃ alkyl)carbamate;
(ii)

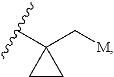

wherein M is selected from the group consisting of hydroxy, C₁-C₄ dialkylamino, and C₁-C₄ alkylamino, and wherein the cyclopropyl group is unsubstituted or independently substituted with up to 2 balo groups;
(iii)

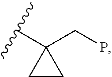

wherein P is a 5- to 10-membered monocyclic, fused bicyclic, bridged bicyclic, or spirocyclic heterocycloalkyl, wherein the 5- to 10-membered monocyclic, fused bicyclic, bridged bicyclic, or spirocyclic heterocycloalkyl is saturated and contains 1 to 2 heteroatoms selected from the group consisting of N. S. and O, wherein the 5- to 10-membered monocyclic, fused bicyclic, bridged bicyclic, or spirocyclic heterocycloalkyl is unsubstituted or substituted with 1 to 2 R^{P} substituents selected from the group consisting of halo, hydroxy, C₁-C₃ alkyl, C₁-C₃ hydroxyalkyl, C₁-C₃ cyanoalkyl, C₁-C₃ fluoroalkyl carbamoyl, C₁-C₃ alkoxy, C₁-C₃ fluoroalkoxy, cyano, C₃-C₆ cycloalkyl, C₃-C₆ fluorocycloalkyl, and —NHC(O)C₁-C₃alkyl, and wherein the cyclopropyl group is unsubstituted or independently substituted with up to 2 halo groups; and
(iv) a 4- to 8-membered monocyclic or fused, bridged or spiro-bicyclic cycloalkyl, wherein the 4- to 8-membered monocyclic or fused, bridged or spiro-bicyclic cycloalkyl is saturated and wherein the 4- to 8-membered monocyclic or fused, bridged or spiro-bicyclic cycloalkyl is unsubstituted or independently substituted with 1 to 3 substituents R^{ZC} selected from the group consisting of halo, hydroxy, C₁-C₃ alkyl, C₁-C₃ fluoroalkyl, C₁-C₃ hydroxyalkyl, C₁-C₃ hydroxyfluoroalkyl, C₃-C₄ cycloalkyl, C₃-C₄ fluorocycloalkyl, C₃-C₄ hydroxycycloalkyl, and C₃-C₄ hydroxyfluorocycloalkyl.

In another embodiment, the present disclosure provides a compound of Formula (I), or the pharmaceutically acceptable salt thereof, wherein Ring Z is selected from the group consisting of:
(i) a 5- to 8-membered monocyclic or fused, bridged or spiro-bicyclic heterocycloalkyl, wherein the 5- to 8-membered monocyclic or fused, bridged or spiro-bicyclic heterocycloalkyl is saturated and contains 1 to 3 heteroatoms independently selected from the group consisting of N, S, and O, and wherein the 5- to 8-membered monocyclic or fused, bridged or spiro-bicyclic heterocycloalkyl is unsubstituted or substituted with 1 to 2 substituents R^{ZHC} selected from the group consisting of halo, hydroxy, C₁-C₃ alkyl, C₁-C₃ hydroxyalkyl, —C(H)(OH)CF₂H, —O—CH₂—O—(C₁-C₃ fluoroalkyl), C₂-C₄ fluoroalkenyl, and methylene (C₁-C₃ alkyl) (C₁-C₃ alkyl) carbamate;
(ii)

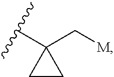

wherein M is selected from the group consisting of hydroxy, C₁-C₄ dialkylamino, and C₁-C₄ alkylamino, and wherein the cyclopropyl group is unsubstituted or independently substituted with up to 2 halo groups;
(iii)

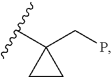

wherein P is a 5- to 8-membered monocyclic, fused bicyclic, or bridged bicyclic heterocycloalkyl, wherein the 5- to 8-membered monocyclic, fused bicyclic, or bridged bicyclic heterocycloalkyl is saturated and contains 1 to 2 heteroatoms selected from the group consisting of N, S, and O, wherein the 5- to 8-membered monocyclic, fused bicyclic, or bridged bicyclic heterocycloalkyl is unsubstituted or substituted with 1 to 2 R^{P} substituents selected from the group consisting of halo, hydroxy, C₁-C₃ alkyl, C₁-C₃ hydroxyalkyl, C₁-C₃ cyanoalkyl, C₁-C₃ fluoroalkyl carbamoyl, C₁-C₃ alkoxy, C₁-C₃ fluoroalkoxy, cyano, C₃-C₆ cycloalkyl, C₃-C₆ fluorocycloalkyl, and —NHC(O)C₁-C₃alkyl, and wherein the cyclopropyl group is unsubstituted or independently substituted with up to 2 halo groups; and
(iv) a 4- to 8-membered monocyclic or fused, bridged or spiro-bicyclic cycloalkyl, wherein the 4- to 8-membered monocyclic or fused, bridged or spiro-bicyclic cycloalkyl is saturated and wherein the 4- to 8-membered monocyclic or fused, bridged or spiro-bicyclic cycloalkyl is unsubstituted or independently substituted with 1 to 3 substituents R^{ZC} selected from the group consisting of halo, hydroxy, C₁-C₃ alkyl, C₁-C₃ fluoroalkyl, C₁-C₃ hydroxyalkyl, C₁-C₃ hydroxyfluoroalkyl, C₃-C₄ cycloalkyl, C₃-C₄ fluorocycloalkyl, C₃-C₄ hydroxycycloalkyl, and C₃-C₄ hydroxyfluorocycloalkyl.

In another embodiment, the present disclosure provides a compound of Formula (I), or the pharmaceutically acceptable salt thereof, wherein the group

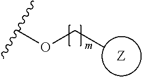

is selected from the group consisting of:
(i)

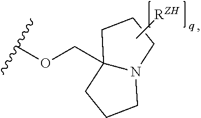

wherein subscript q is 1 or 2;
(ii)

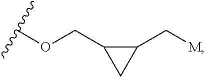

wherein M is selected from the group consisting of hydroxy, C₁-C₃ dialkylamino, and C₁-C₄ alkylamino, and wherein the cyclopropyl group is unsubstituted or substituted with up to 2 fluoro groups; and
(iii)

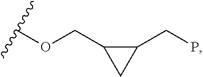

wherein P is a 5- to 8-membered monocyclic, fused bicyclic, or bridged bicyclic heterocycloalkyl, wherein the 5- to 8-membered monocyclic, fused bicyclic, or bridged bicyclic heterocycloalkyl is saturated and contains 1 to 2 heteroatoms selected from the group consisting of N and O, wherein the 5- to 8-membered monocyclic, fused bicyclic, or bridged bicyclic heterocycloalkyl is unsubstituted or substituted with 1 R^{P} substituent selected from the group consisting of halo, hydroxy, C₁-C₃ hydroxyalkyl, C₁-C₃ cyanoalkyl, carbamoyl, C₁-C₃ alkoxy, cyano, and —NHC(O)C₁-C₃alkyl, and wherein the cyclopropyl group is unsubstituted or substituted with up to 2 fluoro groups.

In another embodiment, the present disclosure provides a compound of Formula (I), or the pharmaceutically acceptable salt thereof, wherein Ring Z is selected from the group consisting of:

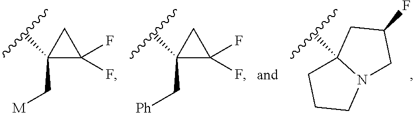

and the subscript m is 1.

In another embodiment, the present disclosure provides a compound of Formula (I), or the pharmaceutically acceptable salt thereof, wherein Ring Z is selected from the group consisting of:

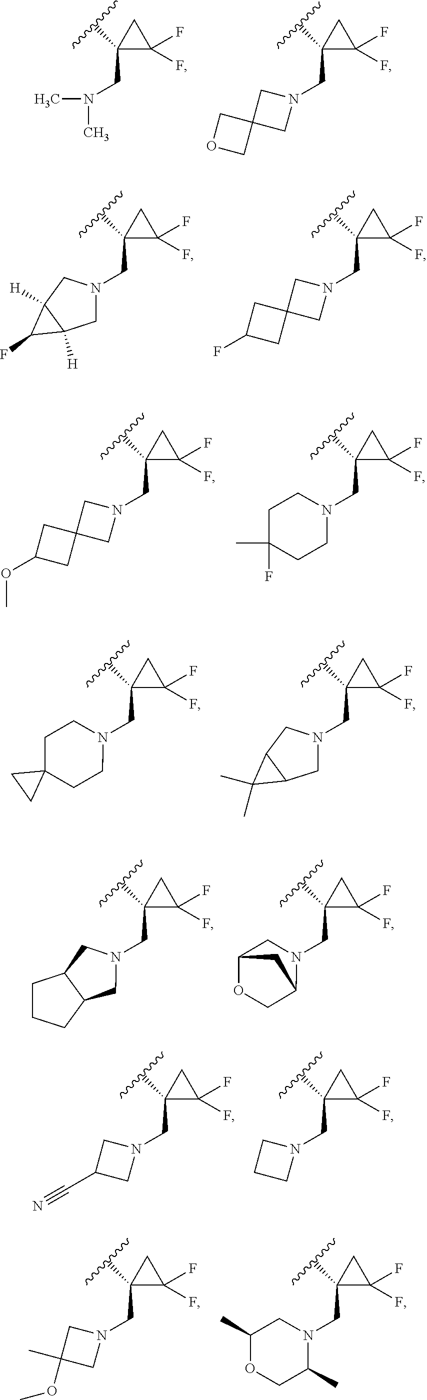

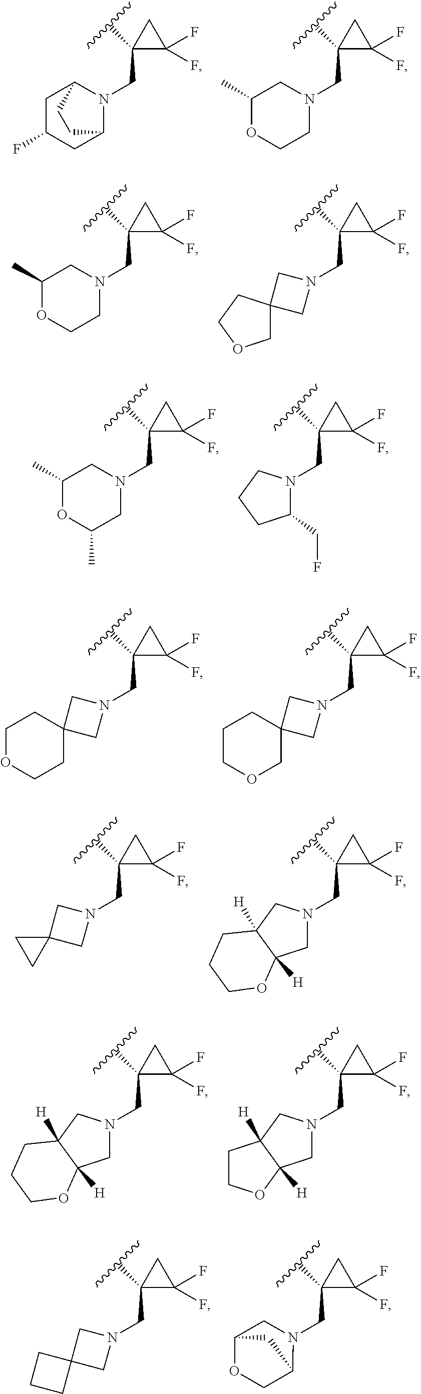

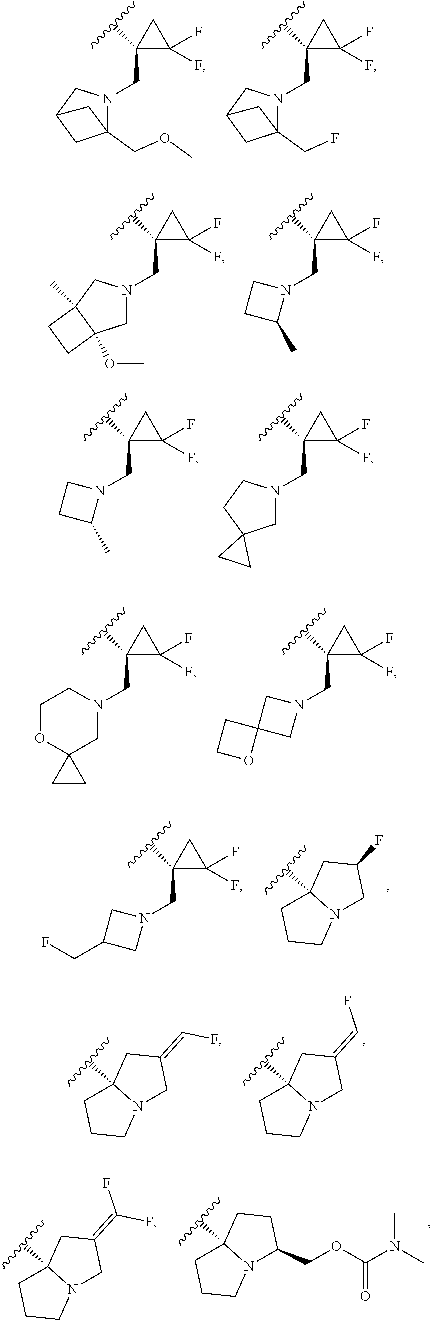

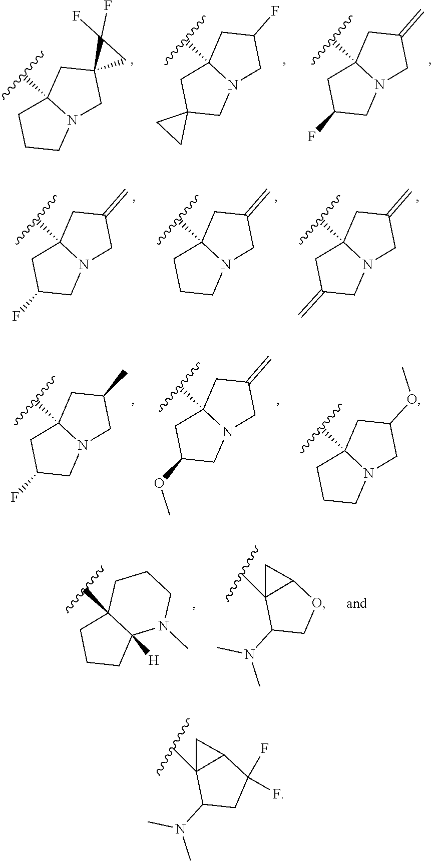

In another embodiment, the present disclosure provides a compound of Formula (I), or the pharmaceutically acceptable salt thereof, wherein Ring Z is selected from the group consisting of:

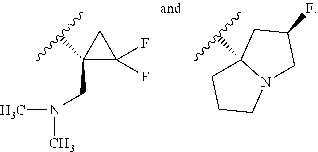

In another embodiment, the present disclosure provides a compound of Formula (I), or the pharmaceutically acceptable salt thereof, wherein the group

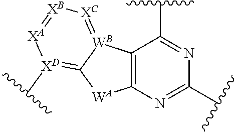

is selected from the group consisting of:

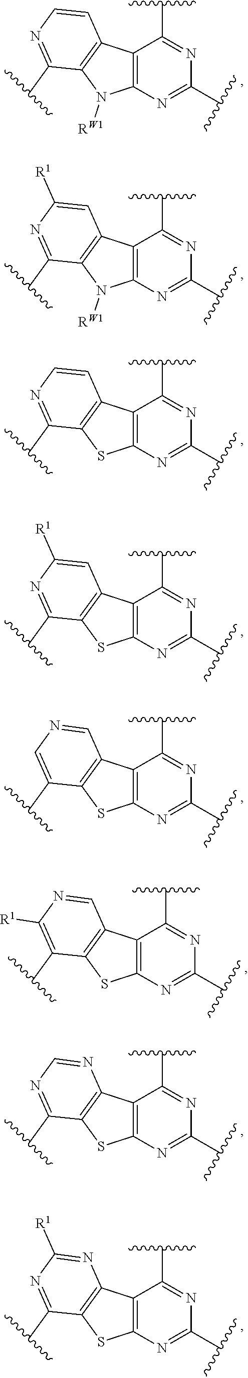

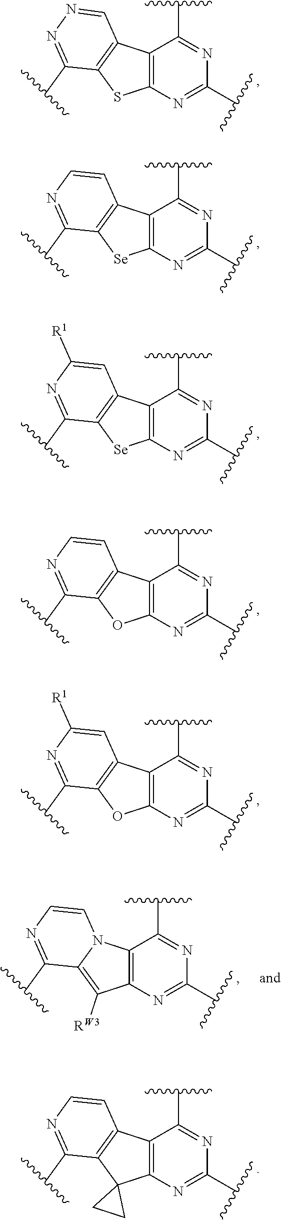

In another embodiment, the present disclosure provides a compound of Formula (I), or the pharmaceutically acceptable salt thereof, wherein the compound of Formula (I) has Formula (IA)

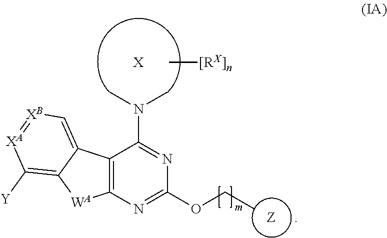

In another embodiment, the present disclosure provides a compound of Formula (I), or the pharmaceutically acceptable salt thereof, wherein the compound of Formula (I) has Formula (IB)

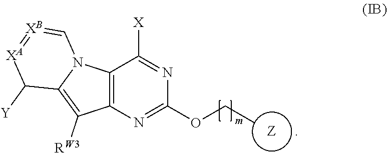

In specific embodiments, the present disclosure provides a compound as described in any one of Examples 1-307 as set forth below, or a pharmaceutically acceptable salt thereof.

In one embodiment, the present disclosure provides a compound having structural Formula (I), or a pharmaceutically acceptable salt thereof, as shown above, wherein:

X is:

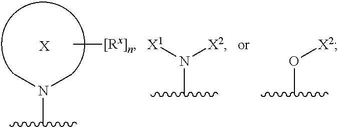

Ring X is selected from the group consisting of:
(i) a 5- to 9-membered monocyclic or fused bicyclic or bridged bicyclic heterocycloalkyl, wherein the 5- to 9-membered monocyclic or fused bicyclic or bridged bicyclic heterocycloalkyl is saturated and contains 0 to 2 heteroatom groups selected from the group consisting of N, S, S(O), S(O)₂ and O, in addition to the illustrated N atom; and
(ii) an 8- to 10-membered spiroheterocycloalkyl, wherein the 8- to 10-membered spiroheterocycloalkyl is saturated and contains 0 to 2 heteroatom groups selected from the group consisting of N, S, S(O), S(O)₂ and O, in addition to the illustrated N atom;
each R^{X} is independently selected from the group consisting of fluoro, cyano, hydroxy, oxo, C₁-C₆ alkyl, C₁-C₆ fluoroalkyl, C₁-C₆ alkoxy, C₁-C₆ fluoroalkoxy, C₁-C₆ cyanoalkyl, and C₁-C₆ hydroxyalkyl;
X¹ is selected from the group consisting of H, C₁-C₆ alkyl, C₁-C₆ fluoroalkyl, C₁-C₆ fluoroalkoxy, C₃-C₆ cycloalkyl, and C₃-C₆ fluorocycloalkyl;
x² is H or —(CR^{a}₂)ₚ—C^{X}, wherein C^{X} is:
   (i) a 3- to 10-membered monocyclic or bicyclic cycloalkyl or an 8- to 10-membered spirocycloalkyl;
   (ii) a 3- to 10-membered monocyclic or bicyclic heterocycloalkyl or an 8- to 10-membered spiroheterocycloalkyl, wherein the 3- to 10-membered monocyclic or bicyclic heterocycloalkyl or the 8- to 10-membered spiroheterocycloalkyl is saturated and contains 1 to 3 heteroatom groups independently selected from the group consisting of N, S, S(O), S(O)₂ and O;
      wherein C^{X} is unsubstituted or substituted by 1 to 2 RCA substituents independently selected from the group consisting of fluoro, hydroxy, oxo, cyano, C₁-C₆ alkyl, C₁-C₆ acyl, C₁-C₆ fluoroalkyl, C₁-C₆ alkoxy, C₁-C₆ fluoroalkoxy, C₁-C₆ hydroxyalkyl, C₁-C₆ cyanoalkyl, C₃-C₆ cycloalkyl, and C₃-C₆ fluorocycloalkyl;
each R^{a} is independently selected from the group consisting of hydrogen, halo, cyano, hydroxy, C₁-C₆ alkyl, C₁-C₆ fluoroalkyl, C₁-C₆ alkoxy, C₁-C₆ fluoroalkoxy, C₁-C₆ cyanoalkyl, C₁-C₆ fluorocyanoalkyl, C₃-C₆ cycloalkyl, and C₃-C₆ fluorocycloalkyl;
X^{A}, X^{B}, and X^{C}, are independently selected from the group consisting of N, C(H), and C(R¹);
X^{D} is selected from the group consisting of N and C;
each R¹ is independently selected from the group consisting of C₁-C₃ alkyl, C₁-C₃ fluoroalkyl, C₁-C₃ alkoxy, C₁-C₃ fluoroalkoxy, halo, hydroxy, oxo, cyano, C₁-C₃ alkylamino, C₁-C₃ dialkylamino, and C^{B};
   wherein C^{B} is a monocyclic, saturated heterocycloalkyl containing 1 to 3 heteroatoms independently selected from the group consisting of N, S, and O;
      wherein C^{B} is unsubstituted or substituted by 1 to 2 R^{C}^{^{B}} substituents independently selected from the group consisting of halo, hydroxy, oxo, cyano, C₁-C₃ alkyl, C₁-C₃ fluoroalkyl, C₁-C₃ alkoxy, C₁-C₃ fluoroalkoxy, C₃-C₆ cycloalkyl, and C₃-C₆ fluorocycloalkyl;
W^{A} is selected from the group consisting of N(R^{W1}), C(R^{W2})₂, O, S, and C(R^{W3});
R^{W1} is selected from the group consisting of C₁-C₃ alkyl, C₁-C₃ fluoroalkyl, C₃-C₆ cycloalkyl, and C₃-C₆ fluorocycloalkyl;
each R^{W2} is independently selected from the group consisting of fluoro, C₁-C₃ alkyl, C₁-C₃ fluoroalkyl, and hydroxy;
or alternatively, the two R^{W2}, together with the carbon atom to which they are attached form a C₃-C₆ cycloalkyl or C₃-C₆ fluorocycloalkyl;
R^{W3} is selected from the group consisting of H, halo, C₁-C₃ alkyl, C₁-C₃ alkoxy, C₁-C₃ fluoroalkyl, C₁-C₃ fluoroalkoxy, C₃-C₆ cycloalkyl, and C₃-C₆ fluorocycloalkyl;
W^{B} is C or N, wherein when:
   W^{B} is C, then W^{A} is N(R^{W1}), C(R^{W2})₂, O, or S; and
   W^{B} is N, then W^{A} is C(R^{W3});
Y is selected from the group consisting of C₁-C₆ alkyl, C₁-C₆ fluoroalkyl, and C^{Y};
C^{Y} is:
   (i) an aryl selected from the group consisting of phenyl and naphthyl:
   (ii) a 5- to 6-membered monocyclic heteroaryl, wherein the 5- to 6-membered monocyclic heteroaryl contains 1 to 3 heteroatoms independently selected from the group consisting of N, S, and O; or
   (iii) a 9- to 10-membered bicyclic heteroaryl, wherein the 9- to 10-membered bicyclic heteroaryl contains 1 to 3 heteroatoms independently selected from the group consisting of N, O, and S; or
   (iv) a 12- to 17-membered tri- or tetracyclic heterocycloalkyl, where at least 2 of the rings of the 12- to 17-membered tri- or tetracyclic heterocycloalkyl are aromatic, the third ring is partially unsaturated or aromatic, and the fourth ring, if present, is saturated, wherein the 12- to 17-membered tri- or tetracyclic heterocycloalkyl contains 1 to 4 heteroatoms independently selected from the group consisting of N, S, and O;
      wherein C^{Y} is unsubstituted or substituted by 1 to 4 RY substituents independently selected from the group consisting of halo, hydroxy, cyano, C₁-C₆ alkyl, C₂-C₆ alkynyl, C₂-C₇ alkenyl, C₁-C₆ fluoroalkyl, C₁-C₆ alkoxy, C₁-C₆ haloalkoxy, C₂-C₆ fluoroalkynyl, C₂-C₇ fluoroalkenyl, C₁-C₆ alkylthio, C₁-C₆ fluoroalkylthio, amino, C₁-C₆ alkylamino, C₁-C₆ dialkylamino, tri (C₁-C₆ alkyl) silyl, cyano, C₁-C₆ cyanoalkyl, C₁-C₆ fluorocyanoalkyl, and R^{ya};
         wherein R^{ya} is:
         (a) a 3- to 9-membered monocyclic or bicyclic cycloalkyl;
         (b) a 3- to 9-membered monocyclic or bicyclic cycloalkenyl;
         (c) a 5- to 6-membered heteroaryl containing 1 to 3 heteroatoms independently selected from the group consisting of N, S, and O; or a 4- to 7-membered saturated heterocycloalkyl containing 1 to 2 (d) heteroatoms independently selected from the group of N, S, and O;
         wherein R^{ya} is unsubstituted or substituted by 1 to 2 substituents independently selected from the group consisting of halo, hydroxy, cyano, C₁-C₃ alkyl, C₁-C₃ fluoroalkyl, C₃-C₆ cycloalkyl, C₃-C₆ fluorocycloalkyl, C₁-C₃ alkoxy, C₁-C₃ fluoroalkoxy, and C₁-C₃ cyanoalkyl;
Ring Z is selected from the group consisting of:
   (i) a 3- to 10-membered monocyclic, bicyclic, or tricyclic heterocycloalkyl, wherein the 3- to 10-membered monocyclic, bicyclic, or tricyclic heterocycloalkyl is saturated or partially unsaturated and contains 1 to 3 heteroatoms independently selected from the group consisting of N, S, and O; and
   (ii) a 3- to 10-membered monocyclic, bicyclic, or tricyclic cycloalkyl, wherein the 3- to 10-membered monocyclic, bicyclic, or tricyclic cycloalkyl is saturated or partially unsaturated;
   wherein ring Z is unsubstituted or independently substituted with 1 to 4 substituents R^{ZC} selected from the group consisting of halo, hydroxy, oxo, cyano, C₁-C₃ alkyl, C₁-C₃ fluoroalkyl, C₁-C₃ hydroxyalkyl, C₁-C₃ hydroxyfluoroalkyl, C₁-C₃ alkoxy, C₁-C₃ fluoroalkoxy, C₁-C₃ cyanoalkyl, C₃-C₆ cycloalkyl, C₃-C₆ fluorocycloalkyl, C₃-C₆ hydroxycycloalkyl, C₃-C₆ hydroxyfluorocycloalkyl, C₂-C₄ fluoroalkenyl, C₁-C₃ alkylamino, C₁-C₃ dialkylamino, methylene (C₁-C₃ alkyl)amino, methylenedi (C₁-C₃ alkyl)amino, and methylene (C₁-C₃ alkyl) (C₁-C₃ alkyl) carbamate;
   wherein ring Z is optionally substituted with 1 P or —CH₂P, wherein P is a 4- to 10-membered monocyclic, bicyclic, or tricyclic heterocycloalkyl, wherein the 4- to 10-membered monocyclic, bicyclic, or tricyclic heterocycloalkyl is saturated or partially unsaturated and contains 1 to 2 heteroatoms selected from the group consisting of N, S, and O;
   wherein P is unsubstituted or independently substituted with 1 to 4 R^{P} substituents selected from the group consisting of halo, hydroxy, oxo, cyano, C₁-C₃ alkyl, C₁-C₃ fluoroalkyl, C₁-C₃ hydroxyalkyl, C₁-C₃ hydroxyfluoroalkyl, C₁-C₃ alkoxy, C₁-C₃ fluoroalkoxy, C₁-C₃ cyanoalkyl, C₃-C₆ cycloalkyl, C₃-C₆ fluorocycloalkyl, C₃-C₆ hydroxycycloalkyl, C₃-C₆ hydroxyfluorocycloalkyl, carbamoyl, and —NHC(O)C₁-C₃alkyl;
subscript m is 0, 1, or 2;
subscript n is 0, 1, 2, or 3; and
subscript p is 0, 1, 2, or 3.

The present disclosure includes the pharmaceutically acceptable salts of the compounds defined herein, including the pharmaceutically acceptable salts of all structural formulas, embodiments and classes defined herein.

### Definitions

Unless defined otherwise, all technical and scientific terms used herein have the same meaning as is commonly understood by one of skill in the art to which this invention belongs.

As used throughout this disclosure, “compound(s) of Formula (I)”, “compound(s) disclosed herein”, “compound(s) described herein”, “compound(s) of the disclosure”, etc., are used interchangeably and are to be understood to include the disclosed compounds of Formula (I). The compounds of Formula (I) can form salts which are also within the scope of the present disclosure. Reference to a compound of the disclosure (or compound of Formula (I)) herein is understood to include reference to salts thereof, unless otherwise indicated.

“Acyl” refers to a moiety derived by the removal of one or more hydroxyl groups from an oxoacid. An acyl group contains a central carbon atom, a double-bonded oxygen atom to the central carbon atom, and a single-bonded alkyl group to the central carbon atom.

“Alkenyl” means an aliphatic hydrocarbon group containing at least one carbon-carbon double bond and which may be straight or branched. Non-limiting examples include ethenyl, propenyl, and butenyl.

“Alkenylenyl” or “alkenylene” means a divalent group derived from an alkenyl. “Fluoroalkenylenyl” means an alkenylenyl that is mono- or multiple-fluoro-substituted.

“Alkyl”, as well as other groups having the prefix “alk”, such as alkoxy, and the like, means carbon chains which may be linear or branched, or combinations thereof, containing the indicated number of carbon atoms. For instance, a C₁-C₆ alkyl means an alkyl group having one (i.e., methyl) up to 6 carbon atoms (i.e., hexyl). In particular embodiments, linear alkyl groups have 1-6 carbon atoms and branched alkyl groups have 3-7 carbon atoms. Examples of alkyl groups include methyl, ethyl, propyl, isopropyl, butyl, sec- and tert-butyl, pentyl, hexyl, heptyl, octyl, nonyl and the like.

“Alkylenyl” or “alkylene,” means a divalent group derived from an alkyl. An example of an alkylene group include methylenyl.

“Alkylenealkylamino” means an alkylamino group linked to an alkylene group. The bond to the parent moiety is through a carbon atom of the alkylene group.

“Alkylenedialkylamino” means a dialkylamino group linked to an alkylene group. The bond to the parent moiety is through a carbon atom of the alkylene group.

“Alkylenealkylalkylcarbamate” means a carbamate group (having two alkyl groups attached to the nitrogen atom) linked to an alkylene group. The bond to the parent moiety is through a carbon atom of the alkylene group. For example, methylene alkylalkylcarbamate has the structure of

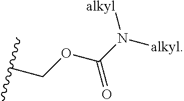

In other words, the carbamate group has alkyl groups, which can be the same or different, as previously defined, attached to the nitrogen atom.

“Alkylamino” means one or two alkyl groups linked to an amino group. The bond to the parent moiety is through a nitrogen atom of the amino component.

“Alkylthio” means an alkyl group linked to a sulfur. “Fluoroalkylthio” means an alkylthio that is mono- or multiple-fluoro-substituted.

“Alkoxy” and “alkyl-O)—” are used interchangeably and refer to an alkyl group linked to oxygen. “Haloalkoxy” means an alkoxy that is mono- or multiple-halo-substituted. The halo groups on a multiple-halo-substituted alkoxy group can be the same or different.

“Alkoxyalkyl” means an alkoxy group linked to an alkyl group. The bond to the parent moiety is through a carbon atom of the alkyl component.

“Alkoxycarbonyl” means an alkoxy group linked to a carbonyl group. The bond to the parent moiety is through a carbon atom of the carbonyl component.

“Alkynyl” means an aliphatic hydrocarbon group containing at least one carbon-carbon triple bond and which may be straight or branched. Non-limiting examples include ethynyl, propynyl, and butynyl.

“Aryl” means a monocyclic, bicyclic, tricyclic, or tetracyclic carbocyclic aromatic ring or ring system containing 5-17 carbon atoms, wherein at least one of the rings is aromatic. Non-limiting examples include phenyl and naphthyl.

“Bicyclic ring system” refers to two joined rings. “Tricyclic ring system” refers to three joined rings. “Tetracyclic ring system” refers to four joined rings. The rings may be fused, i.e., share two adjacent atoms, or “spirocyclic”, i.e., share only a single atom, or “bridged”, i.e., share three or more atoms with two bridgehead atoms being connected by a bridge containing at least one atom. Likewise the bicyclic or tricyclic rings may be aryl rings, heterocyclic rings, cycloalkyl rings, etc.

“Carbamoyl” means a H₂N—C(O)— group, which is the univalent group formed by loss of —OH group of carbamic acid. The bond to the parent group is through the carbon atom of the carbonyl component.

“Cyanoalkyl” means an -alkyl-CN group in which the alkyl is as previously defined. The bond to the parent moiety is through a carbon atom of the alkyl component. Non-limiting examples of suitable cyanoalkyl groups include cyanomethyl and 3-cyanopropyl. “Fluorocyanoalkyl” means a cyanoalkyl that is mono- or multiple-fluoro-substituted.

“Cycloalkyl” means a saturated cyclic hydrocarbon radical. In particular embodiments, the cycloalkyl group has 3-12 carbon atoms, forming 1-3 carbocyclic rings, wherein cyclic systems having 2-3 rings can be fused. Examples of cycloalkyl include cyclopropyl, cyclobutyl, cyclopentyl, cyclohexyl, cycloheptyl, adamantyl, and the like. “Fluorocycloalkyl” means a saturated cyclic hydrocarbon radical that is mono- or multiple-fluoro-substituted, e.g., doubly fluoro-substituted cyclopentyl. “Cycloalkoxy” refers to a cycloalkyl group linked through an oxygen to the parent moiety. “Spirocycloalkyl” means a saturated spirocyclic hydrocarbon radical having at least two rings sharing only a single atom.

“Cycloalkenyl” means a non-saturated cyclic hydrocarbon radical containing at least one carbon-carbon double bond. “Spirocycloalkenyl” means a non-saturated spirocyclic hydrocarbon radical (containing at least one carbon-carbon double bond) having at least two rings sharing only a single atom.

“Dialkylamino” means an alkylamino as previously defined, wherein the amino atom is substituted by two alkyl substituents, which substitutions can be the same or different, e.g., —N(CH₃)₂ or —N(CH₃)(CH₂CH₃).

“Fluoroalkyl” includes mono-substituted as well as multiple fluoro-substituted alkyl groups, up to perfluoro substituted alkyl. For example, fluoromethyl, 1,1-difluoroethyl, trifluoromethyl or 1,1,1,2,2-pentafluorobutyl are included. “Fluoroalkenyl” includes mono-substituted as well as multiple fluoro-substituted alkenyl groups. “Fluoroalkynyl” includes mono-substituted as well as multiple fluoro-substituted alkynyl groups. “Fluoroalkoxy” includes mono-substituted as well as multiple fluoro-substituted “alkoxy” groups as previously defined. “Hydroxyfluoroalkyl” includes mono-substituted as well as multiple fluoro-substituted hydroxyalkyl groups. “Hydroxyfluorocycloalkyl” includes mono-substituted as well as multiple fluoro-substituted hydroxycycloalkyl groups.

“Halogen” or “halo”, unless otherwise indicated, includes fluorine (fluoro), chlorine (chloro), bromine (bromo) and iodine (iodo). In one embodiment, halo is fluoro (—F) or chloro (—Cl).

“Heteroaryl” refers to aromatic monocyclic, bicyclic and tricyclic ring structures in which one or more atoms in the ring, the heteroatom(s), is an element other than carbon. Heteroatoms are typically O, S, or N atoms. Examples of heteroaryl groups include pyrazolyl, oxadiazolonyl, pyridinyl, pyrimidinyl, pyrrolyl, pyridazinyl, isoxazolyl, thiazolyl, oxazolyl, indolyl, benzoxazolyl, benzothiazolyl, and imidazolyl.

“Heterocycloalkyl” or “heterocyclic ring” or “heterocycle” means a non-aromatic monocyclic, bicyclic, tricyclic or bridged ring system comprising about 3 to about 10 ring atoms, preferably about 5 to about 10 ring atoms, in which one or more of the atoms in the ring system is an element other than carbon, for example, nitrogen, oxygen, phosphorus or sulfur, alone or in combination. There are no adjacent oxygen and/or sulfur atoms present in the ring system. In some embodiments, heterocycloalkyls contain about 5 to about 6 ring atoms. The prefix aza, oxa, phospha or thia before the heterocyclyl root name means that at least a nitrogen, oxygen, phosphorus or sulfur atom respectively is present as a ring atom. In some embodiments, the nitrogen or sulfur atom of the heterocycloalkyl can be optionally oxidized to the corresponding N-oxide, S-oxide or S,S-dioxide. For instance, in some embodiments the heterocycloalkyl can contain N, S, S(O), S(O)₂ and/or O (which are referred to herein as “heteroatom groups”). Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, phosphorinane, phosphinane, 1-oxophosphinan-1-ium and the like. “Spirobeterocycloalkyl” refers to a fused ring system in which the rings share only a single atom and at least one of the rings is a heterocycloalkyl.

“Hydroxyalkyl” means a HO-alkyl-group in which alkyl is as previously defined. The bond to the parent moiety is through a carbon atom of the alkyl group. Preferred hydroxyalkyls contain lower alkyl. Non-limiting examples of suitable hydroxyalkyl groups include hydroxymethyl and 2-hydroxyethyl. “Hydroxyfluoroalkyl” means a HO-fluoroalkyl- group in which fluoroalkyl is as previously defined. “Hydroxycycloalkyl” means a HO-cycloalkyl-group in which cycloalkyl is as previously defined. “Hydroxyfluorocycloalkyl” means a HO-fluorocycloalkyl-group in which fluorocycloalkyl is as previously defined.

“Trialkylsilyl” means a silicon radical having three alkyl groups covalently bonded to the silicon atom.

When any variable (e.g., R^{X}) occurs more than one time in any constituent or in Formula (I) or other generic formulas herein, its definition on each occurrence is independent of its definition at every other occurrence. Combinations of substituents and/or variables are permissible only if such combinations result in stable compounds. In choosing compounds of the present disclosure, one of ordinary skill in the art will recognize that the various substituents, e.g., R^{X}, are to be chosen in conformity with well-known principles of chemical structure connectivity and stability. Unless expressly stated to the contrary, substitution by a named substituent is permitted on any atom in a ring (e.g., aryl, a heteroaryl ring, or a saturated heteroaryl ring) provided such ring substitution is chemically allowed and results in a stable compound. A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

Unless expressly depicted or described otherwise, variables depicted in a structural formula with a “floating” bond, such as R^{X}, are permitted on any available carbon atom in the ring to which the variable is attached. When a moiety is noted as being “optionally substituted” in Formula (I) or any embodiment thereof, it means that Formula (I) or the embodiment thereof encompasses compounds that contain the noted substituent (or substituents) on the moiety and also compounds that do not contain the noted substituent (or substituents) on the moiety.

The wavy line [symbol] , as used herein, indicates a point of attachment to the rest of the compound.

The compounds of Formula (I) may contain one or more asymmetric centers and can thus occur as racemates and racemic mixtures, single enantiomers, diastereoisomeric mixtures and individual diastereoisomers. Centers of asymmetry that are present in the compounds of Formula (I) can all independently of one another have S configuration or R configuration. The compounds of Formula (I) include all possible enantiomers and diastereomers and mixtures of two or more stereoisomers, for example, mixtures of enantiomers and/or diastereomers, in all ratios. Thus, enantiomers are a subject of the disclosure in enantiomerically pure form, both as levorotatory and as dextrorotatory antipodes, in the form of racemates and in the form of mixtures of the two enantiomers in all ratios. In the case of a cis/trans isomerism, the disclosure includes both the cis form and the trans form as well as mixtures of these forms in all ratios. The present disclosure is meant to comprehend all such stereoisomeric forms of the compounds of Formula (I). Where a structural formula or chemical name specifies a particular configuration at a stereocenter, the enantiomer or stereoisomer of the compound resulting from that specified stereocenter is intended. Where a structural formula of the compounds of Formula (I) indicates a straight line at a chiral center, the structural formula includes both the S and R stereoisomers associated with the chiral center and mixtures thereof.

The compounds of Formula (I) may be separated into their individual diastereoisomers by, for example, fractional crystallization from a suitable solvent, for example, methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration. Vibrational circular dichroism (VCD) may also be used to determine the absolute stereochemistry. Alternatively, any stereoisomer or isomers of the compounds of Formula (I) may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereoisomeric mixture, followed by separation of the individual diastereoisomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

The compounds of Formula (I) which contain olefinic double bonds, unless specified otherwise, they are meant to include both E and Z geometric isomers.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed by the compounds of Formula (I).

Some of the compounds of Formula (I) described herein may exist as atropisomers when the rotational energy barrier around a single bond is sufficiently high to prevent free rotation at a given temperature, thus allowing isolation of individual conformers with distinct properties. The individual atropisomers as well as mixtures thereof are encompassed with compounds of Formula (I) of the present disclosure. When resolved, individual atropisomers can be designated by established conventions such as those specified by the International Union of Pure Applied Chemistry (IUPAC) 2013 Recommendations.

In the compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure as described and claimed herein is meant to include all suitable isotopic variations of the compounds of Formula (I) and embodiments thereof. For example, different isotopic forms of hydrogen (H) include protium (¹H) and deuterium (²H, also denoted herein as D). Protium is the predominant hydrogen isotope found in nature. Enriching for deuterium may afford certain therapeutic advantages, such as increasing in vivo half-life or reducing dosage requirements or may provide a compound useful as a standard for characterization of biological samples. Isotopically-enriched compounds can be prepared without undue experimentation by conventional techniques well known to those skilled in the art or by processes analogous to those described in the Schemes and Examples herein using appropriate isotopically-enriched reagents and/or intermediates.

The term “pharmaceutically acceptable salts” refers to salts prepared from pharmaceutically acceptable non-toxic bases or acids. When a compound of Formula (I) is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Preferred are the ammonium, calcium, magnesium, potassium and sodium salts. Salts prepared from pharmaceutically acceptable organic non-toxic bases include salts of primary, secondary, and tertiary amines derived from both naturally occurring and synthetic sources. Pharmaceutically acceptable organic non-toxic bases from which salts can be formed include, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, dicyclohexylamine, lysine, methylglucamine, morpholine, piperazine, piperidine, polyamine resins, procaine, purines, theobromine, triethylamine, trimethylamine, tripropylamine, tromethamine and the like.

When a compound of Formula (I) is basic, its corresponding salt can be conveniently prepared from pharmaceutically acceptable non-toxic inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids. If a compound of Formula (I) simultaneously contains acidic and basic groups in the molecule, the disclosure also includes, in addition to the salt forms mentioned, inner salts or betaines (zwitterions). Salts can be obtained from the compounds of Formula (I) by customary methods which are known to the person skilled in the art, for example, by combination with an organic or inorganic acid or base in a solvent or dispersant, or by anion exchange or cation exchange from other salts. The present disclosure also includes all salts of the compounds of Formula (I) which, owing to low physiological compatibility, are not directly suitable for use in pharmaceuticals but which can be used, for example, as intermediates for chemical reactions or for the preparation of pharmaceutically acceptable salts.

Furthermore, the compounds of Formula (I) may exist in amorphous form and/or one or more crystalline forms, and as such all amorphous and crystalline forms and mixtures thereof of the compounds of Formula (I), including the Examples, are intended to be included within the scope of the present disclosure. In addition, some of the compounds of Formula (I) may form solvates with water (i.e., a hydrate) or common organic solvents such as but not limited to ethyl acetate. Such solvates and hydrates, particularly the pharmaceutically acceptable solvates and hydrates, of the instant compounds are likewise encompassed within the scope of this disclosure, along with un-solvated and anhydrous forms.

Any pharmaceutically acceptable pro-drug modification of a compound of Formula (I) which results in conversion in vivo to a compound within the scope of this disclosure is also within the scope of this disclosure.

The terms “therapeutically effective (or efficacious) amount” and similar descriptions such as “an amount efficacious for treatment” or “an effective dose” are intended to mean that amount of a compound of Formula (I) that will elicit the biological or medical response of a tissue, a system, animal or human that is being sought by a researcher, veterinarian, medical doctor or other clinician. In a preferred embodiment, the term “therapeutically effective amount” means an amount of a compound of Formula (I) that alleviates at least one clinical symptom in a human patient. The terms “prophylactically effective (or efficacious) amount” and similar descriptions such as “an amount efficacious for prevention” are intended to mean that amount of a compound of Formula (I) that will prevent or reduce the risk of occurrence of the biological or medical event that is sought to be prevented in a tissue, a system, animal or human by a researcher, veterinarian, medical doctor or other clinician.

### Dosages of the Compounds of Formula (I)

The dosage regimen utilizing a compound of Formula (I) is selected in accordance with a variety of factors including type, species, age, weight, sex and medical condition of the patient; the severity of the condition to be treated; the potency of the compound chosen to be administered; the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition. It is understood that a specific daily dosage amount can simultaneously be both a therapeutically effective amount, e.g., for treatment of an oncological condition, and a prophylactically effective amount, e.g., for prevention of an oncological condition.

While individual needs vary, determination of optimal ranges of effective amounts of the compounds of Formula (I) is within the skill of the art. For administration to a human in, for example, the curative or prophylactic treatment of the conditions and disorders identified herein, the typical dosages of the compounds of Formula (I) can be about 0.05 mg/kg/day to about 50 mg/kg/day, or at least 0.05 mg/kg, or at least 0.08 mg/kg, or at least 0.1 mg/kg, or at least 0.2 mg/kg, or at least 0.3 mg/kg, or at least 0.4 mg/kg, or at least 0.5 mg/kg, and any amount therebetween, to about 50 mg/kg or less, or about 40 mg/kg or less, or about 30 mg/kg or less, or about 20 mg/kg or less, or about 10 mg/kg or less and any amount therebetween, which can be, for example, about 2.5 mg/day (0.5 mg/kg×5 kg) to about 5000 mg/day (50 mg/kg×100 kg). For example, dosages of the compounds can be about 0.1 mg/kg/day to about 50 mg/kg/day, or about 0.05 mg/kg/day to about 10 mg/kg/day, or about 0.05 mg/kg/day to about 5 mg/kg/day, or about 0.05 mg/kg/day to about 3 mg/kg/day, or about 0.07 mg/kg/day to about 3 mg/kg/day, or about 0.09 mg/kg/day to about 3 mg/kg/day, or about 0.05 mg/kg/day to about 0.1 mg/kg/day, or about 0.1 mg/kg/day to about 1 mg/kg/day, or about 1 mg/kg/day to about 10 mg/kg/day, or about 1 mg/kg/day to about 5 mg/kg/day, or about 1 mg/kg/day to about 3 mg/kg/day, or about 3 mg/day to about 500 mg/day, or about 5 mg/day to about 250 mg/day, or about 10 mg/day to about 100 mg/day, or about 3 mg/day to about 10 mg/day, or about 100 mg/day to about 250 mg/day. Such doses may be administered in a single dose or may be divided into multiple doses.

### Pharmaceutical Compositions

The compounds of Formula (I) and their pharmaceutically acceptable salts can be administered to animals, preferably to mammals, and in particular to humans, as pharmaceuticals by themselves, in mixtures with one another or in the form of pharmaceutical compositions. The term “subject” or “patient” includes animals, preferably mammals and especially humans, who use the instant active agents for the prevention or treatment of a medical condition. Administering of the drug to the subject includes both self-administration and administration to the patient by another person. The subject may be in need of, or desire, treatment for an existing disease or medical condition, or may be in need of or desire prophylactic treatment to prevent or reduce the risk of occurrence of said disease or medical condition. As used herein, a subject “in need” of treatment of an existing condition or of prophylactic treatment encompasses both a determination of need by a medical professional as well as the desire of a patient for such treatment.

The present disclosure therefore also provides the compounds of Formula (I) and their pharmaceutically acceptable salts for use as pharmaceuticals, their use for modulating the activity of mutant and/or WT KRAS proteins and in particular their use in the therapy and prophylaxis of the below-mentioned diseases or disorders as well as their use for preparing medicaments for these purposes. In certain embodiments, the compounds of Formula (I) and their pharmaceutically acceptable salts inhibit the KRAS-G12C, KRAS-G12D, KRAS-G12V, and/or KRAS-G13D proteins.

Furthermore, the present disclosure provides pharmaceutical compositions which comprise as active component an effective dose of at least one compound of Formula (I) and/or a pharmaceutically acceptable salt thereof and a customary pharmaceutically acceptable carrier, i.e., one or more pharmaceutically acceptable carrier substances and/or additives.

Thus, the present disclosure provides, for example, said compound and its pharmaceutically acceptable salts for use as pharmaceutical compositions which comprise as active component an effective dose of at least one compound of Formula (I) and/or a pharmaceutically acceptable salt thereof and a customary pharmaceutically acceptable carrier, and the uses of said compound and/or a pharmaceutically acceptable salt thereof in the therapy or prophylaxis of the below-mentioned diseases or disorders, e.g., cancer, as well as their use for preparing medicaments for these purposes.

The pharmaceutical compositions according to the disclosure can be administered orally, for example, in the form of pills, tablets, lacquered tablets, sugar-coated tablets, granules, hard and soft gelatin capsules, aqueous, alcoholic or oily solutions, syrups, emulsions or suspensions, or rectally, for example, in the form of suppositories. Administration can also be carried out parenterally, for example subcutaneously, intramuscularly or intravenously in the form of solutions for injection or infusion.

Other suitable administration forms are, for example, percutaneous or topical administration, for example, in the form of ointments, tinctures, sprays or transdermal therapeutic systems, or, for example, microcapsules, implants or rods. The preferred administration form depends, for example, on the disease to be treated and on its severity.

The amount of active compound of a compound described herein and/or its pharmaceutically acceptable salts in the pharmaceutical composition normally is from 0.01 to 200 mg, or from 0.1 to 200 mg, or from 1 to 200 mg, per dose, but depending on the type of the pharmaceutical composition, it can also be higher. In some embodiments, the amount of active compound of a compound of Formula (I) and/or its pharmaceutically acceptable salts in the pharmaceutical composition is from 0.01 to 10 mg per dose. The pharmaceutical compositions usually comprise 0.5 to 90 percent by weight of at least one compound of Formula (I) and/or its pharmaceutically acceptable salts. The preparation of the pharmaceutical compositions can be carried out in a manner known per se. For this purpose, one or more compounds of Formula (I) and/or their pharmaceutically acceptable salts, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical in human or veterinary medicine.

For the production of pills, tablets, sugar-coated tablets and hard gelatin capsules, it is possible to use, for example, lactose, starch, for example, maize starch, or starch derivatives, talc, stearic acid or its salts, etc. Carriers for soft gelatin capsules and suppositories are, for example, fats, waxes, semisolid and liquid polyols, natural or hardened oils, etc. Suitable carriers for the preparation of solutions, for example, of solutions for injection, or of emulsions or syrups are, for example, water, physiologically acceptable sodium chloride solution, alcohols such as ethanol, glycerol, polyols, sucrose, invert sugar, glucose, mannitol, vegetable oils, etc. It is also possible to lyophilize the compounds of Formula (I) and their pharmaceutically acceptable salts and to use the resulting lyophilisates, for example, for preparing preparations for injection or infusion. Suitable carriers for microcapsules, implants or rods are, for example, copolymers of glycolic acid and lactic acid.

Besides the active compounds and carriers, the pharmaceutical compositions can also contain customary additives, for example, fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents and/or antioxidants.

### Methods of Using the Compounds of Formula (I)

The present application provides a method of inhibiting RAS-mediated cell signaling comprising contacting a cell with a compound of Formula (I) or a pharmaceutically acceptable salt thereof. Inhibition of RAS-mediated signal transduction can be assessed and demonstrated by a wide variety of ways known in the art. Non-limiting examples include (a) a decrease in GTPase activity of RAS; (b) a decrease in GTP binding affinity or an increase in GDP binding affinity; (c) an increase in K_{off} of GTP or a decrease in K_{off} of GDP; (d) a decrease in the levels of signaling transduction molecules downstream in the RAS pathway, such as a decrease in pMEK, pERK, or pAKT levels; and/or (c) a decrease in binding of RAS complex to downstream signaling molecules including but not limited to Raf. Kits and commercially available assays can be utilized for determining one or more of the above.

The present application also provides methods of using the compounds of Formula (I) (or their pharmaceutically acceptable salts) or pharmaceutical compositions containing such compounds to treat disease conditions, including but not limited to, conditions implicated by mutant KRAS proteins and/or amplification or over expression of WT KRAS protein (e.g., cancer), and in some embodiments the KRAS-G12C, KRAS-G12D, KRAS-G12V, and/or KRAS-G13D mutants.

In some embodiments, a method for treatment of cancer is provided, the method comprising administering a therapeutically effective amount a compound of Formula (I) (or a pharmaceutically acceptable salt thereof) or any of the foregoing pharmaceutical compositions comprising such a compound to a subject in need of such treatment. In some embodiments, the cancer is mediated by a KRAS mutation, e.g., the KRAS-G12C, KRAS-G12D, KRAS-G12V, and/or KRAS-G13D mutations. In various embodiments, the cancer is pancreatic cancer, colorectal cancer or lung cancer. In some embodiments, the cancer is gall bladder cancer, thyroid cancer, or bile duct cancer.

In some embodiments the present disclosure provides a method of treating a disorder in a subject in need thereof, wherein said method comprises determining if the subject has a KRAS mutation (e.g., KRAS-G12C, KRAS-G12D, KRAS-G12V, and/or KRAS-G13D mutations) and if the subject is determined to have the KRAS mutation, then administering to the subject a therapeutically effective amount of a compound of Formula (I) or a pharmaceutically acceptable salt thereof.

In some embodiments the present disclosure provides a method of treating a disorder in a subject in need thereof, wherein said method comprises determining if the subject has amplified and/or over expression of WT KRAS protein and if the subject is determined to have such features, then administering to the subject a therapeutically effective amount of a compound of Formula (I) or a pharmaceutically acceptable salt thereof.

The disclosed compounds inhibit anchorage-independent cell growth and therefore have the potential to inhibit tumor metastasis. Accordingly, another embodiment of the present disclosure provides a method for inhibiting tumor metastasis, the method comprising administering an effective amount a compound of Formula (I).

KRAS mutations have also been identified in hematological malignancies (e.g., cancers that affect blood, bone marrow and/or lymph nodes). Accordingly, certain embodiments are directed to administration of the compounds of Formula (I) (e.g., in the form of a pharmaceutical composition) to a subject in need of treatment of a hematological malignancy. Such malignancies include, but are not limited to leukemias and lymphomas. For example, the presently disclosed compounds can be used for treatment of diseases such as acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMoL) and/or other leukemias. In other embodiments, the compounds are useful for treatment of lymphomas such as Hodgkin's lymphoma or non-Hodgkin's lymphoma. In various embodiments, the compounds are useful for treatment of plasma cell malignancies such as multiple myeloma, mantle cell lymphoma, and Waldenstrom's macroglubunemia.

Determining whether a tumor or cancer comprises a KRAS mutation (e.g., the KRAS-G12C, KRAS-G12D and/or KRAS-G12V mutations) or WT KRAS can be undertaken by assessing the nucleotide sequence encoding the KRAS protein, by assessing the amino acid sequence of the KRAS protein, or by assessing the characteristics of a putative KRAS mutant or WT KRAS protein. The sequence of wild-type human KRAS is known in the art.

Methods for detecting a mutation in a KRAS nucleotide sequence or a WT KRAS nucleotide sequence are also known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for KRAS mutations (e.g., the KRAS-G12C, KRAS-G12D, KRAS-G12V, and/or KRAS-G13D mutations) by real-time PCR. In real-time PCR, fluorescent probes specific for the KRAS mutation are used. When a mutation is present, the probe binds and fluorescence is detected. In some embodiments, the KRAS mutation is identified using a direct sequencing method of specific regions (e.g., exon 2 and/or exon 3) in the KRAS gene.

Methods for detecting a mutation in a KRAS protein or a WT KRAS protein (e.g., the KRAS-G12C, KRAS-G12D, KRAS-G12V, KRAS-G13D mutations) are known by those of skill in the art. These methods include, but are not limited to, detection of a KRAS mutant or WT KRAS protein using a binding agent (e.g., an antibody) specific for the mutant or WT protein, protein electrophoresis and Western blotting, and direct peptide sequencing.

A number of tissue samples can be assessed for determining whether a tumor or cancer comprises a KRAS mutation (e.g., the KRAS-G12C, KRAS-G12D, KRAS-G12V, and/or KRAS-G13D mutations) or amplified/overexpressed WT KRAS. In some embodiments, the sample is taken from a subject baving a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is a circulating tumor cell (CTC) sample. In some embodiments, the sample is processed to a cell lysate. In some embodiments, the sample is processed to DNA or RNA.

The present application also provides a method of treating a hyperproliferative disorder comprising administering a therapeutically effective amount of a compound of Formula (I), or a pharmaceutically acceptable salt thereof to a subject in need thereof. In some embodiments, said method relates to the treatment of a subject who suffers from a cancer such as acute myeloid leukemia, cancer in adolescents, adrenocortical carcinoma childhood, AIDS-related cancers (e.g., lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, Burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin's lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer; multiple endocrine neoplasia syndromes, multiple myeloma/plasma cell neoplasm, mycosis fungoides, myelodysplasia syndromes, myelodysplastic/myeloproliferative neoplasms, multiple myeloma, Merkel cell carcinoma, malignant mesothelioma, malignant fibrous histiocytoma of bone and osteosarcoma, nasal cavity and paranasal sinus cancer, nasopharyngeal cancer, neuroblastoma, non-Hodgkin's lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer; small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer. In some embodiments, said method relates to the treatment of a non-cancerous hyperproliferative disorder such as benign hyperplasia of the skin (e.g., psoriasis), restenosis, or prostate (e.g., benign prostatic hypertrophy (BPH)).

In some embodiments, the methods for treatment are directed to treating lung cancers, and the methods comprise administering a therapeutically effective amount of the compounds of Formula (I) (or pharmaceutical composition comprising such compounds) to a subject in need thereof. In certain embodiments, the lung cancer is a non-small cell lung carcinoma (NSCLC), for example, adenocarcinoma, squamous-cell lung carcinoma or large-cell lung carcinoma. In some embodiments, the lung cancer is a small cell lung carcinoma. Other lung cancers which the compounds of Formula (I) may provide therapeutic benefit for include, but are not limited to, glandular tumors, carcinoid tumors and undifferentiated carcinomas.

The present disclosure also provides methods of modulating a mutant KRAS protein activity (e.g., activity resulting from the KRAS-G12C, KRAS-G12D, KRAS-G12V, and/or KRAS-G13D mutations) or a WT KRAS protein activity by contacting the protein with an effective amount of a compound of Formula (I). Modulation can be inhibiting or activating protein activity. In some embodiments, the present disclosure provides methods of inhibiting protein activity by contacting the mutant KRAS protein (e.g., KRAS-G12C, KRAS-G12D, KRAS-G12V, and/or KRAS-G13D mutants) or WT KRAS protein with an effective amount of a compound of Formula (I) in solution. In some embodiments, the present disclosure provides methods of inhibiting the mutant or WT KRAS protein activity by contacting a cell, tissue, or organ that expresses the protein of interest. In some embodiments, the disclosure provides methods of inhibiting protein activity in subjects including, but not limited to, rodents and mammals (e.g., humans) by administering into the subjects an effective amount of a compound of Formula (I).

### Combination Therapies

One or more additional pharmacologically active agents may be administered in combination with a compound of Formula (I) (or a pharmaceutically acceptable salt thereof). An additional active agent (or agents) is intended to mean a pharmaceutically active agent (or agents) that is active in the body, including pro-drugs that convert to pharmaceutically active form after administration, which are different from the compound of Formula (I). The additional active agents also include free-acid, free-base and pharmaceutically acceptable salts of said additional active agents. Generally, any suitable additional active agent or agents, including chemotherapeutic agents or therapeutic antibodies, may be used in any combination with the compound of Formula (I) in a single dosage formulation (e.g., a fixed dose drug combination), or in one or more separate dosage formulations which allows for concurrent or sequential administration of the active agents (co-administration of the separate active agents) to subjects. In addition, the compounds of Formula (I) (or pharmaceutically acceptable salts thereof) can be administered in combination with radiation therapy, hormone therapy, surgery or immunotherapy.

The present application also provides methods for combination therapies in which the additional active agent is known to modulate other pathways, or other components of the same pathway, or even overlapping sets of target enzymes which are used in combination with a compound of Formula (I), or a pharmaceutically acceptable salt thereof. In one embodiment, such therapy includes, but is not limited to, the combination of one or more compounds of Formula (I) with chemotherapeutic agents, immunotherapeutic agents, hormonal and anti-hormonal agents, targeted therapy agents, and anti-angiogenesis agents, to provide a synergistic or additive therapeutic effect. In another embodiment, such therapy includes radiation treatment to provide a synergistic or additive therapeutic effect.

Examples of additional active agents (i.e., additional anti-cancer agents) include chemotherapeutic agents (e.g., cytotoxic agents), immunotherapeutic agents, hormonal and anti-hormonal agents, targeted therapy agents, and anti-angiogenesis agents. Many anti-cancer agents can be classified within one or more of these groups. While certain anti-cancer agents have been categorized within a specific group(s) or subgroup(s) herein, many of these agents can also be listed within one or more other group(s) or subgroup(s), as would be presently understood in the art. It is to be understood that the classification herein of a particular agent into a particular group is not intended to be limiting. Many anti-cancer agents are presently known in the art and can be used in combination with the compounds of the present disclosure.

Further, an agent can be an agonist, antagonist, allosteric modulator, toxin or, more generally, may act to inhibit or stimulate its target (e.g., receptor or enzyme activation or inhibition). For example, suitable for use are one or more agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor “c-met”.

In an embodiment, the additional anti-cancer agent is a chemotherapeutic agent, an immunotherapeutic agent, a hormonal agent, an anti-hormonal agent, a targeted therapy agent, or an anti-angiogenesis agent (or angiogenesis inhibitor). In an embodiment, the additional anti-cancer agent is selected from the group consisting of a chemotherapeutic agent, a mitotic inhibitor, a plant alkaloid, an alkylating agent, an anti-metabolite, a platinum analog, an enzyme, a topoisomerase inhibitor, a retinoid, an aziridine, an antibiotic, a hormonal agent, an anti-hormonal agent, an anti-estrogen, an anti-androgen, an anti-adrenal, an androgen, a targeted therapy agent, an immunotherapeutic agent, a biological response modifier, a cytokine inhibitor, a tumor vaccine, a monoclonal antibody, an immune checkpoint inhibitor, an anti-PD-1 agent, an anti-PD-L1 agent, a colony-stimulating factor, an immunomodulator, an immunomodulatory imide (IMID), an anti-CTLA4 agent, an anti-LAG1 agent, an anti-LAG3 agent, an anti-ILT4 agent, an anti-OX40 agent, a GITR agonist, a CAR-T cell, a BITE, a signal transduction inhibitor, a growth factor inhibitor, a tyrosine kinase inhibitor, an EGFR inhibitor, a histone deacetylase (HDAC) inhibitor, a proteasome inhibitor, a cell-cycle inhibitor, an anti-angiogenesis agent, a matrix-metalloproteinase (MMP) inhibitor, a hepatocyte growth factor inhibitor, a TOR inhibitor, a KDR inhibitor, a VEGF inhibitor, a HIF-1α inhibitor, a HIF-2α inhibitor, a fibroblast growth factor (FGF) inhibitor, a RAF inhibitor, a MEK inhibitor, an ERK inhibitor, a PI3K inhibitor, an AKT inhibitor, an MCL-1 inhibitor, a BCL-2 inhibitor, an SHP2 inhibitor, a HER-2 inhibitor, a BRAF-inhibitor, a gene expression modulator, an autophagy inhibitor, an apoptosis inducer, an antiproliferative agent, and a glycolysis inhibitor.

In one embodiment, the additional anti-cancer agent(s) is a chemotherapeutic agent. Non-limiting examples of chemotherapeutic agents include mitotic inhibitors and plant alkaloids, alkylating agents, anti-metabolites, platinum analogs, enzymes, topoisomerase inhibitors, retinoids, aziridines, and antibiotics.

Non-limiting examples of mitotic inhibitors and plant alkaloids include taxanes such as cabazitaxel, docetaxel, larotaxel, ortataxel, paclitaxel, and tesetaxel; demecolcine; epothilone; eribulin; etoposide (VP-16); etoposide phosphate; navelbine; noscapine; teniposide; thaliblastine; vinblastine; vincristine; vindesine; vinflunine; and vinorelbine.

Non-limiting examples of alkylating agents include nitrogen mustards such as chlorambucil, chlomaphazine, cholophosphamide, cytophosphane, estramustine, ifosfamide, mannomustine, mechlorethamine, mechlorethamine oxide hydrochloride, melphalan, novembichin, phenesterine, prednimustine, tris(2-chloroethyl)amine, trofosfamide, and uracil mustard; alkyl sulfonates such as busulfan, improsulfan, and piposulfan; nitrosoureas such as carmustine, chlorozotocin, fotemustine, lomustine, nimustine, ranimustine, streptozotocin, and TA-07; ethylenimines and methylamelamines such as altretamine, thiotepa, triethylenemelamine, triethylenethiophosphaoramide, trietylenephosphoramide, and trimethylolomelamine; ambamustine; bendamustine; dacarbazine; etoglucid; irofulven; mafosfamide; mitobronitol; mitolactol; pipobroman; procarbazine; temozolomide; treosulfan; and triaziquone.

Non-limiting examples of anti-metabolites include folic acid analogues such as aminopterin, denopterin, edatrexate, methotrexate, pteropterin, raltitrexed, and trimetrexate; purine analogs such as 6-mercaptopurine, 6-thioguanine, fludarabine, forodesine, thiamiprine, and thioguanine; pyrimidine analogs such as 5-fluorouracil (5-FU), 6-azauridine, ancitabine, azacytidine, capecitabine, carmofur, cytarabine, decitabine, dideoxyuridine, doxifiuridine, doxifluridine, enocitabine, floxuridine, galocitabine, gemcitabine, and sapacitabine; 3-aminopyridine-2-carboxaldehyde thiosemicarbazone; broxuridine; cladribine; cyclophosphamide; cytarabine; emitefur; hydroxyurea; mercaptopurine; nelarabine; pemetrexed; pentostatin; tegafur; and troxacitabine.

Non-limiting examples of platinum analogs include carboplatin, cisplatin, dicycloplatin, heptaplatin, lobaplatin, nedaplatin, oxaliplatin, satraplatin, and triplatin tetranitrate.

Non-limiting examples of enzymes include asparaginase and pegaspargase.

Non-limiting examples of topoisomerase inhibitors include acridine carboxamide, amonafide, amsacrine, belotecan, elliptinium acetate, exatecan, indolocarbazole, irinotecan, lurtotecan, mitoxantrone, razoxane, rubitecan, SN-38, sobuzoxane, and topotecan.

Non-limiting examples of retinoids include alitretinoin, bexarotene, fenretinide, isotretinoin, liarozole, RII retinamide, and tretinoin.

Non-limiting examples of aziridines include benzodopa, carboquone, meturedopa, and uredopa.

Non-limiting examples of antibiotics include intercalating antibiotics; anthracenediones; anthracycline antibiotics such as aclarubicin, amrubicin, daunomycin, daunorubicin, doxorubicin, epirubicin, idarubicin, menogaril, nogalamycin, pirarubicin, and valrubicin; 6-diazo-5-oxo-L-norleucine; aclacinomysins; actinomycin; authramycin; azaserine; bleomycins; cactinomycin; calicheamicin; carabicin; carminomycin; carzinophilin; chromomycins; dactinomycin; detorubicin; esorubicin; esperamicins; geldanamycin; marcellomycin; mitomycins; mitomycin C; mycophenolic acid; olivomycins; novantrone; peplomycin; porfiromycin; potfiromycin; puromycin; quelamycin; rebeccamycin; rodorubicin; streptonigrin; streptozocin; tanespimycin; tubercidin; ubenimex; zinostatin; zinostatin stimalamer; and zorubicin.

In one embodiment, the additional anti-cancer agent(s) is a hormonal and/or anti-hormonal agent (i.e., hormone therapy). Non-limiting examples of hormonal and anti-hormonal agents include anti-androgens such as abiraterone, apalutamide, bicalutamide, darolutamide, enzalutamide, flutamide, goserelin, leuprolide, and nilutamide; anti-estrogens such as 4-hydroxy tamoxifen, aromatase inhibiting 4 (5)-imidazoles, EM-800, fosfestrol, fulvestrant, keoxifene, LY 117018, onapristone, raloxifone, tamoxifen, toremifene, and trioxifene; anti-adrenals such as aminoglutethimide, dexaminoglutethimide, mitotane, and trilostane; androgens such as calusterone, dromostanolone propionate, epitiostanol, mepitiostane, and testolactone; abarelix; anastrozole; cetrorelix; deslorelin; exemestane; fadrozole; finasteride; formestane; histrelin (RL 0903); human chorionic gonadotropin; lanreotide; LDI 200 (Milkhaus); letrozole; leuprorelin; mifepristone; nafarelin; nafoxidine; osaterone; prednisone; thyrotropin alfa; and triptorelin.

In one embodiment, the additional anti-cancer agent(s) is an immunotherapeutic agent (i.e., immunotherapy). Non-limiting examples of immunotherapeutic agents include biological response modifiers, cytokine inhibitors, tumor vaccines, monoclonal antibodies, immune checkpoint inhibitors, colony-stimulating factors, and immunomodulators.

Non-limiting examples of biological response modifiers, including cytokine inhibitors (cytokines) such as interferons and interleukins, include interferon alfa/interferon alpha such as interferon alfa-2, interferon alfa-2a, interferon alfa-2b, interferon alfa-n1, interferon alfa-n3, interferon alfacon-1, peginterferon alfa-2a, peginterferon alfa-2b, and leukocyte alpha interferon; interferon beta such as interferon beta-1a, and interferon beta-1b; interferon gamma such as natural interferon gamma-1a, and interferon gamma-1b; aldesleukin; interleukin-1 beta; interleukin-2; oprelvekin; sonermin; tasonermin; and virulizin.

Non-limiting examples of tumor vaccines include APC 8015, A VICINE, bladder cancer vaccine, cancer vaccine (Biomira), gastrin 17 immunogen, Maruyama vaccine, melanoma lysate vaccine, melanoma oncolysate vaccine (New York Medical College), melanoma vaccine (New York University), melanoma vaccine (Sloan Kettering Institute), TICE® BCG (Bacillus Calmette-Guerin), and viral melanoma cell lysates vaccine (Royal Newcastle Hospital).

Non-limiting examples of monoclonal antibodies include abagovomab, adecatumumab, aflibercept, alemtuzumab, blinatumomab, brentuximab vedotin, CA 125 MAb (Biomira), cancer MAb (Japan Pharmaceutical Development), daclizumab, daratumumab, denosumab, edrecolomab, gemtuzumab zogamicin, HER-2 and Fc MAb (Medarex), ibritumomab tiuxetan, idiotypic 105AD7 MAb (CRC Technology), idiotypic CEA MAb (Trilex), ipilimumab, quavonlimab, vibostolimab, favezelimab, lintuzumab, LYM-1-iodine 131 MAb (Techni clone), mitumomab, moxetumomab, ofatumumab, polymorphic epithelial mucin-yttrium 90 MAb (Antisoma), ranibizumab, rituximab, and trastuzumab.

Non-limiting examples of immune checkpoint inhibitors include anti-PD-1 agents or antibodies such as cemiplimab, nivolumab, and pembrolizumab; anti-PD-L1 agents or antibodies such as atezolizumab, avelumab, and durvalumab; anti-CTLA-4 agents or antibodies such as ipilumumab and quavonlimab; anti-LAG1 agents; anti-LAG3 agents such as favezelimab, and anti-OX40 agents.

Non-limiting examples of colony-stimulating factors include darbepoetin alfa, epoctin alfa, epoetin beta, filgrastim, granulocyte macrophage colony stimulating factor, lenograstim, leridistim, mirimostim, molgramostim, nartograstim, pegfilgrastim, and sargramostim.

Non-limiting examples of additional immunotherapeutic agents include BiTEs, CAR-T cells, GITR agonists, imiquimod, immunomodulatory imides (IMiDs), mismatched double stranded RNA (Ampligen), resiquimod, SRL 172, and thymalfasin.

In one embodiment, the additional anti-cancer agent(s) is a targeted therapy agent (i.e., targeted therapy). Targeted therapy agents include, for example, monoclonal antibodies and small molecule drugs. Non-limiting examples of targeted therapy agents include signal transduction inhibitors, growth factor inhibitors, tyrosine kinase inhibitors, EGFR inhibitors, histone deacetylase (HDAC) inhibitors, proteasome inhibitors, cell-cycle inhibitors, angiogenesis inhibitors, matrix-metalloproteinase (MMP) inhibitors, hepatocyte growth factor inhibitors, TOR inhibitors, KDR inhibitors, VEGF inhibitors, fibroblast growth factors (FGF) inhibitors, MEK inhibitors, ERK inhibitors, PI3K inhibitors, AKT inhibitors, MCL-1 inhibitors, BCL-2 inhibitors, SHP2 inhibitors, HER-2 inhibitors, BRAF-inhibitors, BTK inhibitors (e.g., nemtabrutinib), gene expression modulators, autophagy inhibitors, apoptosis inducers, antiproliferative agents, and glycolysis inhibitors.

Non-limiting examples of signal transduction inhibitors include tyrosine kinase inhibitors, multiple-kinase inhibitors, anlotinib, avapritinib, axitinib, dasatinib, dovitinib, imatinib, lenvatinib, lonidamine, nilotinib, nintedanib, pazopanib, pegvisomant, ponatinib, vandetanib, and EGFR inhibitory agents.

Non-limiting examples of EGFR inhibitory agents include small molecule antagonists of EGFR such as afatinib, brigatinib, erlotinib, gefitinib, lapatinib, and osimertinib; and antibody-based EGFR inhibitors, including any anti-EGFR antibody or antibody fragment that can partially or completely block EGFR activation by its natural ligand. Antibody-based EGFR inhibitory agents may include, for example, those described in Modjtahedi, H., et al., 1993, Br. J. Cancer 67:247-253; Teramoto, T., et al., 1996, Cancer 77:639-645; Goldstein et al, 1995, Clin. Cancer Res. 1:1311-1318; Huang, S. M., et al., 1999, Cancer Res. 15: 59 (8): 1935-40; and Yang, X., et al., 1999, Cancer Res. 59:1236-1243; monoclonal antibody Mab E7.6.3 (Yang, 1999 supra); Mab C225 (ATCC Accession No. HB-8508), or an antibody or antibody fragment having the binding specificity thereof; specific antisense nucleotide or siRNA; afatinib, cetuximab; matuzumab; necitumumab; nimotuzumab; panitumumab; and zalutumumab.

Non-limiting examples of histone deacetylase (HDAC) inhibitors include belinostat, panobinostat, romidepsin, and vorinostat.

Non-limiting examples of proteasome inhibitors include bortezomib, carfilzomib, ixazomib, marizomib (salinosporamide a), and oprozomib.

Non-limiting examples of cell-cycle inhibitors, including CDK inhibitors, include abemaciclib, alvocidib, palbociclib, and ribociclib.

In one embodiment, the additional anti-cancer agent(s) is an anti-angiogenic agent (or angiogenesis inhibitor) including, but not limited to, matrix-metalloproteinase (MMP) inhibitors; VEGF inhibitors; EGFR inhibitors; TOR inhibitors such as everolimus and temsirolimus; PDGFR kinase inhibitory agents such as crenolanib; HIF-1α inhibitors such as PX 478; HIF-2α inhibitors such as belzutifan and the HIF-2α inhibitors described in WO 2015/035223; fibroblast growth factor (FGF) or FGFR inhibitory agents such as B-FGF and RG 13577; hepatocyte growth factor inhibitors; KDR inhibitors; anti-Ang1 and anti-Ang2 agents; anti-Tie2 kinase inhibitory agents; Tek antagonists (US 2003/0162712; U.S. Pat. No. 6,413,932); anti-TWEAK agents (U.S. Pat. No. 6,727,225); ADAM distintegrin domain to antagonize the binding of integrin to its ligands (US 2002/0042368); anti-eph receptor and/or anti-ephrin antibodies or antigen binding regions (U.S. Pat. Nos. 5,981,245; 5,728,813; 5,969,110; 6,596,852; 6,232,447; and 6,057,124); and anti-PDGF-BB antagonists as well as antibodies or antigen binding regions specifically binding to PDGF-BB ligands.

Non-limiting examples of matrix-metalloproteinase (MMP) inhibitors include MMP-2 (matrix-metalloproteinase 2) inhibitors, MMP-9 (matrix-metalloproteinase 9) inhibitors, prinomastat, RO 32-3555, and RS 13-0830. Examples of useful matrix metalloproteinase inhibitors are described, for example, in WO 96/33172, WO 96/27583. EP 1004578, WO 98/07697, WO 98/03516, WO 98/34918, WO 98/34915, WO 98/33768, WO 98/30566, EP 0606046, EP 0931788, WO 90/05719, WO 99/52910, WO 99/52889, WO 99/29667, WO 1999/007675, EP 1786785, EP 1181017, US 2009/0012085, U.S. Pat. Nos. 5,863,949, 5,861,510, and EP 0780386. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13).

Non-limiting examples of VEGF and VEGFR inhibitory agents include bevacizumab, cediranib, CEP 7055, CP 547632, KRN 633, orantinib, pazopanib, pegaptanib, pegaptanib octasodium, semaxanib, sorafenib, sunitinib, VEGF antagonist (Borean, Denmark), and VEGF-TRAP™.

The additional anti-cancer agent(s) may also be another anti-angiogenic agent including, but not limited to, 2-methoxyestradiol, AE 941, alemtuzumab, alpha-D148 Mab (Amgen, US), alphastatin, anecortave acetate, angiocidin, angiogenesis inhibitors, (SUGEN, US), angiostatin, anti-Vn Mab (Crucell, Netherlands), atiprimod, axitinib, AZD 9935, BAY RES 2690 (Bayer, Germany, BC 1 (Genoa Institute of Cancer Research, Italy), beloranib, benefin (Lane Labs, US), cabozantinib, CDP 791 (Celltech Group, UK), chondroitinase AC, cilengitide, combretastatin A4 prodrug, CP 564959 (OSI, US), CV247, CYC 381 (Harvard University, US), E 7820, EHT 0101, endostatin, enzastaurin hydrochloride, ER-68203-00 (IVAX, US), fibrinogen-E fragment, Flk-1 (ImClone Systems, US), forms of FLT 1 (VEGFR 1), FR-111142, GCS-100, GW 2286 (GlaxoSmithKline, UK), IL-8, ilomastat, IM-862, irsogladine, KM-2550 (Kyowa Hakko, Japan), lenalidomide, lenvatinib, MAb alpha5beta3 integrin, second generation (Applied Molecular Evolution, USA and Medimmune, US), MAb VEGF (Xenova, UK), marimastat, maspin (Sosei, Japan), metastatin, motuporamine C, M-PGA, ombrabulin, OXI4503, PI 88, platelet factor 4, PPI 2458, ramucirumab, rBPI 21 and BPI-derived antiangiogenic (XOMA, US), regorafenib, SC-236, SD-7784 (Pfizer, US), SDX 103 (University of California at San Diego, US), SG 292 (Telios, US), SU-0879 (Pfizer, US), TAN-1120, TBC-1635, tesevatinib, tetrathiomolybdate, thalidomide, thrombospondin 1 inhibitor, Tie-2 ligands (Regeneron, US), tissue factor pathway inhibitors (EntreMed, US), tumor necrosis factor-alpha inhibitors, tumstatin, TZ 93, urokinase plasminogen activator inhibitors, vadimezan, vandetanib, vasostatin, vatalanib, VE-cadherin-2 antagonists, xanthorrhizol, XL 784 (Exelixis, US), ziv-aflibercept, and ZD 6126.

In embodiments, the additional anti-cancer agent(s) is an additional active agent that disrupts or inhibits RAS-RAF-ERK or PI3K-AKT-TOR signaling pathways or is a PD-1 and/or PD-L1 antagonist. In embodiments, the additional anti-cancer agent(s) is a RAF inhibitor, EGFR inhibitor, MEK inhibitor, ERK inhibitor, PI3K inhibitor, AKT inhibitor, TOR inhibitor, MCL-1 inhibitor, BCL-2 inhibitor, SHP2 inhibitor, proteasome inhibitor, or immune therapy, including monoclonal antibodies, immunomodulatory imides (IMiDs), anti-PD-1, anti-PDL-1, anti-CTLA4, anti-LAG1, anti-LAG3, and anti-OX40 agents, GITR agonists, CAR-T cells, and BiTEs.

Non-limiting examples of RAF inhibitors include dabrafenib, encorafenib, regorafenib, sorafenib, and vemurafenib.

Non-limiting examples of MEK inhibitors include binimctinib, CI-1040, cobimetinib, PD318088, PD325901, PD334581, PD98059, refametinib, selumetinib, and trametinib.

Non-limiting examples of ERK inhibitors include LY3214996, LTT462, MK-8353, SCH772984, ravoxertinib, ulixertinib, and an ERKi as described in WO 2017/068412.

Non-limiting examples of PI3K inhibitors include 17-hydroxywortmannin analogs (e.g., WO 06/044453); AEZS-136; alpelisib; AS-252424; buparlisib; CAL263; copanlisib; CUDC-907; dactolisib (WO 06/122806); demethoxyviridin; duvelisib; GNE-477; GSK1059615; IC87114; idelalisib; INK1117; LY294002; Palomid 529; paxalisib; perifosine; PI-103; PI-103 hydrochloride; pictilisib (e.g., WO 09/036,082; WO 09/055,730); PIK 90; PWT33597; SF1126; sonolisib; TGI 00-115; TGX-221; XL147; XL-765; wortmannin; and ZSTK474.

Non-limiting examples of AKT inhibitors include Akt-1-1 (inhibits Akt1) (Bamett et al. (2005) Biochem. J., 385 (Pt. 2), 399-408); Akt-1-1,2 (Barnett et al. (2005) Biochem. J. 385 (Pt. 2), 399-408); API-59CJ-Ome (e.g., Jin et al. (2004) Br. J. Cancer 91, 1808-12); 1-H-imidazo[4,5-c]pyridinyl compounds (e.g., WO05011700); indole-3-carbinol and derivatives thereof (e.g., U.S. Pat. No. 6,656,963; Sarkar and Li (2004) J Nutr. 134 (12 Suppl), 3493S-3498S); perifosine, Dasmahapatra et al. (2004) Clin. Cancer Res. 10 (15), 5242-52, 2004); phosphatidylinositol ether lipid analogues (e.g., Gills and Dennis (2004) Expert. Opin. Investig. Drugs 13, 787-97); triciribine (Yang et al. (2004) Cancer Res. 64, 4394-9); imidazooxazone compounds including trans-3-amino-1-methyl-3-[4-(3-phenyl-5H-imidazo[1,2-c]pyrido[3,4-e][1,3]oxazin-2-yl)phenyl]-cyclobutanol hydrochloride (WO 2012/137870); afuresertib; capivasertib; MK2206; patasertib, and those disclosed in WO 2011/082270 and WO 2012/177844.

Non-limiting examples of TOR inhibitors include deforolimus; ATP-competitive TORC1/TORC2 inhibitors, including PI-103, PP242, PP30, and Torin 1; TOR inhibitors in FKBP12 enhancer, rapamycins and derivatives thereof, including temsirolimus, everolimus, WO 9409010; rapalogs, e.g. as disclosed in WO 98/02441 and WO 01/14387, e.g. AP23573, AP23464, or AP23841; 40-(2-hydroxyethyl) rapamycin, 40-[3-hydroxy (hydroxymethyl)methylpropanoate]-rapamycin; 40-epi-(tetrazolyl)-rapamycin (also called ABT578); 32-deoxorapamycin; 16-pentynyloxy-32 (S)-dihydrorapanycin, and other derivatives disclosed in WO 05/005434; derivatives disclosed in U.S. Pat. No. 5,258,389, WO 94/090101, WO 92/05179, U.S. Pat. Nos. 5,118,677, 5,118,678, 5,100,883, 5,151,413, 5,120,842, WO 93/111130, WO 94/02136, WO 94/02485, WO 95/14023, WO 94/02136, WO 95/16691, WO 96/41807, WO 96/41807 and U.S. Pat. No. 5,256,790; and phosphorus-containing rapamycin derivatives (e.g., WO 05/016252).

Non-limiting examples of MCL-1 inhibitors include AMG-176, MIK665, and S63845.

Non-limiting examples of SHP2 inhibitors include SHP2 inhibitors described in WO 2019/167000 and WO 2020/022323.

Additional non-limiting examples of anti-cancer agents that are suitable for use include 2-ethylhydrazide, 2,2′,2″-trichlorotriethylamine, ABVD, aceglatone, acemannan, aldophosphamide glycoside, alpharadin, amifostine, aminolevulinic acid, anagrelide, ANCER, ancestim, anti-CD22 immunotoxins, antitumorigenic herbs, apaziquone, arglabin, arsenic trioxide, azathioprine, BAM 002 (Novelos), bel-2 (Genta), bestrabucil, biricodar, bisantrene, bromocriptine, brostallicin, bryostatin, buthionine sulfoximine, calyculin, cell-cycle nonspecific antineoplastic agents, celmoleukin, clodronate, clotrimazole, cytarabine ocfosfate, DA 3030 (Dong-A), defofamine, denileukin diftitox, dexrazoxane, diaziquone, dichloroacetic acid, dilazep, discodermolide, docosanol, doxercalciferol, edelfosine, eflorithine, EL532 (Elan), elfomithine, elsamitrucin, eniluracil, etanidazole, exisulind, ferruginol, folic acid replenisher such as frolinic acid, gacytosine, gallium nitrate, gimeracil/oteracil/tegafur combination (S-1), glycopine, histamine dihydrochloride, HIT diclofenac, HLA-B7 gene therapy (Vical), human fetal alpha fetoprotein, ibandronate, ibandronic acid, ICE chemotherapy regimen, imexon, iobenguane, IT-101 (CRLX101), laniquidar, LC 9018 (Yakult), leflunomide, lentinan, levamisole+fluorouracil, lovastatin, lucanthone, masoprocol, melarsoprol, metoclopramide, miltefosine, miproxifene, mitoguazone, mitozolomide, mopidamol, motexafin gadolinium, MX6 (Galderma), naloxone+pentazocine, nitracrine, nolatrexed, NSC 631570 octreotide (Ukrain), olaparib, P-30 protein, PAC-1, palifermin, pamidronate, pamidronic acid, pentosan polysulfate sodium, phenamet, picibanil, pixantrone, platinum, podophyllinic acid, porfimer sodium, PSK (Polysaccharide-K), rabbit antithymocyte polyclonal antibody, rasburiembodiment, retinoic acid, rhenium Re 186 etidronate, romurtide, samarium (153 Sm) lexidronam, sizofiran, sodium phenylacetate, sparfosic acid, spirogermanium, strontium-89 chloride, suramin, swainsonine, talaporfin, tariquidar, tazarotene, tegafur-uracil, temoporfin, tenuazonic acid, tetrachlorodecaoxide, thrombopoietin, tin ethyl etiopurpurin, tirapazamine, TLC ELL-12, tositumomab-iodine 131, trifluridine and tipiracil combination, troponin I (Harvard University, US), urethan, valspodar, verteporfin, zoledronic acid, and zosuquidar.

The present disclosure further provides a method for using the compounds of Formula (I) or pharmaceutical compositions provided herein, in combination with radiation therapy to treat cancer. Techniques for administering radiation therapy are known in the art, and these techniques can be used in the combination therapy described herein. The administration of the compound of Formula (I) in this combination therapy can be determined as described herein.

Radiation therapy can be administered through one of several methods, or a combination of methods, including, without limitation, external-beam therapy, internal radiation therapy, implant radiation, stereotactic radiosurgery, systemic radiation therapy, radiotherapy and permanent or temporary interstitial brachy therapy. The term “brachytherapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, 1-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as I-125, 1-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive microspheres.

The present disclosure also provides methods for combination therapies in which the additional active agent is known to modulate other pathways, or other components of the same pathway, or even overlapping sets of target enzymes which are used in combination with a compound of Formula (I), or a pharmaceutically acceptable salt thereof. In one embodiment, such therapy includes, but is not limited to, the combination of one or more compounds of Formula (I) with chemotherapeutic agents, immunotherapeutic agents, hormonal therapy agents, therapeutic antibodies, targeted therapy agents, and radiation treatment, to provide a synergistic or additive therapeutic effect.

The compounds of the disclosure can be used in combination with the agents disclosed herein or other suitable agents, depending on the condition being treated. Hence, in some embodiments the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound of Formula (I) and any of the agents described above can be formulated together in the same dosage form and administered simultaneously. Alternatively, a compound of Formula (I) and any of the agents described above can be simultaneously administered, wherein both the agents are present in separate formulations. In another alternative, a compound of Formula (I) can be administered just followed by and any of the agents described above, or vice versa. In some embodiments of the separate administration protocol, a compound of Formula (I) and any of the agents described above are administered a few minutes apart, or a few hours apart, or a few days apart.

As one aspect of the present disclosure contemplates the treatment of the disease/conditions with a combination of pharmaceutically active compounds that may be administered separately, the disclosure further relates to combining separate pharmaceutical compositions in kit form. The kit comprises two separate pharmaceutical compositions: a compound of Formula (I), and a second pharmaceutical compound. The kit comprises a container for containing the separate compositions such as a divided bottle or a divided foil packet. Additional examples of containers include syringes, boxes, and bags. In some embodiments, the kit comprises directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

The present disclosure also provides for the compound of Formula (I), or the pharmaceutically acceptable salt thereof, for use in therapy, or use of the compound of Formula (I), or the pharmaceutically acceptable salt thereof, in therapy. The present disclosure also provides for the compound of Formula (I), or the pharmaceutically acceptable salt thereof, for use in treating cancer, or use of a compound of Formula (I), or the pharmaceutically acceptable salt thereof, for treating cancer. The present disclosure also provides for the compound of Formula (I), or the pharmaceutically acceptable salt thereof, for the preparation of a medicament for the treatment of cancer, or use of the compound of Formula (I), or the pharmaceutically acceptable salt thereof, for the preparation of a medicament for the treatment of cancer. The present disclosure also provides for the compound of Formula (I), or the pharmaceutically acceptable salt thereof, and an additional anti-cancer agent, for use in the treatment of cancer, or use of the compound of Formula (I), or the pharmaceutically acceptable salt thereof, and the additional anti-cancer agent for treating cancer. The disclosure also provides the compound of Formula (I), or the pharmaceutically acceptable salt thereof, and an additional anti-cancer agent, for the preparation of a medicament for the treatment of cancer, or use of the compound of Formula (I), or the pharmaceutically acceptable salt thereof, and the additional anti-cancer agent, for the preparation of a medicament for the treatment of cancer. The present disclosure also provides for a pharmaceutical composition comprising the compound of Formula (I), or the pharmaceutically acceptable salt thereof, for use in the treatment of cancer, or use of the pharmaceutical composition comprising the compound of Formula (I), or the pharmaceutically acceptable salt thereof, for treating cancer. The present disclosure also provides for a pharmaceutical composition comprising the compound of Formula (I), or the pharmaceutically acceptable salt thereof, and an additional anti-cancer agent, for use in the treatment of cancer, or use of the pharmaceutical composition comprising the compound of Formula (I), or the pharmaceutically acceptable salt thereof, and the additional anti-cancer agent, for treating cancer.

### Methods of Preparing the Compounds of the Disclosure

The compounds described herein can be prepared according to the procedures of the following schemes and examples, using appropriate materials and are further exemplified by the following specific examples. The compounds illustrated in the examples are not, however, to be construed as forming the only genus that is considered as the disclosure. The examples further illustrate details for the preparation of the compounds of the present disclosure. Those skilled in the art will readily understand that known variations of the conditions and processes of the following preparative procedures can be used to prepare these compounds. For instance, in some cases, the order of carrying out the steps of reaction schemes may be varied to facilitate the reaction or to avoid unwanted reaction products. These examples are provided for the purpose of further illustration only and are not intended to be limitations on the disclosure. Any intermediates described below may be referred to herein by their number preceded by “Int-.”

Throughout the synthetic schemes and examples, abbreviations and acronyms may be used with the following meanings unless otherwise indicated: Ac=acetyl; AcO=acetate; AcOH=acetic acid; Ac₂O=acetic anhydride; Am=amyl; APhos Pd G3=[4-(di-tert-butylphosphino)-N,N-dimethylaniline-2-(2′-aminobiphenyl)]palladium (II) methanesulfonate; aq.=aqueous; atm=atmosphere; BAST=bis(2-methoxyethyl)aminosulfur trifluoride; Bn=benzyl; Boc=tert-butyloxycarbonyl; BI-DIME=3-(tert-butyl)-4-(2,6-dimethoxyphenyl)-2,3-dihydrobenzo[d][1,3]oxaphosphole; bippyphos=5-(di-tert-butylphosphaneyl)-1′,3′,5′-triphenyl-1′H-1,4′-bipyrazole; Bodipy-GDP=mixture of ((2R,3S,4R,5R)-5-(2-amino-6-oxo-1,6-dihydro-9H-purin-9-yl)-3-(((2-(3-(5,5-difluoro-7,9-dimethyl-5H-414,514-dipyrrolo[1,2-c: 2′,1′-f][1,3,2]diazaborinin-3-yl) propanamido)ethyl) carbamoyl)oxy)-4-hydroxytetrahydrofuran-2-yl)methyl hydrogen diphosphate and ((2R,3R,4R,5R)-5-(2-amino-6-oxo-1,6-dihydro-9H-purin-9-yl)-4-(((2-(3-(5,5-difluoro-7,9-dimethyl-5H-414,514-dipyrrolo[1,2-c: 2′,1′-f][1,3,2]diazaborinin-3-yl) propanamido)ethyl) carbamoyl)oxy)-3-hydroxytetrahydrofuran-2-yl)methyl hydrogen diphosphate (Invitrogen™, catalog number G22360); BOMCI=((chloromethoxy)methyl)benzene; BOP=benzotriazol-1-yloxytris(dimethylamino)phosphonium hexafluorophosphate; bpy=2,2′-bipyridine; B (pin)=(pinacolato) boron; B2pin2=bis(pinacolato)diboron; Bu=butyl; sBu=sec-butyl; (Bu=tert-butyl; (BuO=tert-butoxide; cataCXium A=di(1-adamantyl)-n-butylphosphine; cataCXium A Pd G2=chloro[(di(1-adamantyl)-N-butylphosphine)-2-(2-aminobiphenyl)]palladium (II); cataCXium A Pd G3=mesylate[(di (1-adamantyl)-n-butylphosphine)-2-(2′-amino-1,1′-biphenyl)]palladium (II); Cbz=benzyloxycarbonyl; CbzCl=benzyl chloroformate; conc.=concentrated; Cp=cyclopentadienyl; CSA=(1R)-(−)-camphor-10-sulfonic acid; Cy=cyclohexyl; DAST=(diethylamino) sulfur trifluoride; dba=dibenzylideneacetone; DCE=1,2-dichloroethane; DCM=dichloromethane; DEAD=diethyl azodicarboxylate; DHP=3,4-dihydro-2H-pyran; DIAD=diisopropyl azodicarboxylate; DIBAL=DIBAL-H=diisobutylaluminum hydride; DIPEA=N,N-diisopropylethylamine; DMA=N,N-dimethylacetamide; DMAP=4-(dimethylamino)pyridine; DME=dimethyl ether; DMF=N,N-dimethylformamide; DMP=dimethyl phthalate; DMP=Dess-Martin periodinane=1,1,1-tris(acetyloxy)-1,1-dihydro-1,2-benziodoxol-3-(1H)-one; DMSO=dimethylsulfoxide; DPPA=diphenylphosphoryl azide; dppf=1,1′-bis(diphenylphosphino)ferrocene; EDTA=ethylenediaminetetraacetic acid; equiv, eq.=equivalent(s); EsCI=ethanesulfonyl chloride; Et=ethyl; EtOAc=ethyl acetate; EtOH=ethanol; Fmoc=fluorenylmethoxycarbonyl, GDP=guanosine diphosphate; GTP=guanosine triphosphate; h=hour; HATU=1-[bis(dimethylamino)methylene]-1H-1,2,3-triazolo[4,5-b]pyridinium 3-oxid hexafluorophosphate; HEPES=4-(2-hydroxyethyl)-1-piperazinecthanesulfonic acid; HMDS=hexamethydisilazane; HMPA=4-hydroxymethylphenoxyacetic acid; HPLC=high pressure liquid chromatography; IBX=2-iodoxybenzoic acid; Int=intermediate; i-Pr==iPr=isopropyl; IPA=1-PrOH=isopropyl alcohol; (Ir[dF(CF₃)ppy]₂(dtbpy)) PF₆=[4,4′-bis(1,1-dimethylethyl)-2,2′-bipyridine-N1,N1′]bis[3,5-difluoro-2-[5-(trifluoromethyl)-2-pyridinyl-N]phenyl-C]iridium (III) hexafluorophosphate; KHMDS=potassium bis(trimethylsilyl)amide; LCMS=liquid chromatography-mass spectrometry; min=minute; LDA=lithium diisopropylamide; LiHIMDS=lithium bis(trimethylsilyl)amide; M=Molar; Martin's sulfurane=bis[α,α-bis(trifluoromethyl)benzenemethanolato]diphenylsulfur; mCPBA=3-chlorobenzoperoxoic acid=m-chloroperoxybenzoic acid; Me=methyl; MeCN, ACN=acetonitrile; MeOH=methanol; MO=methoxy; MOMCI=chloromethyl methyl ether; MPLC=medium pressure liquid chromatography; MS=mass spectrometry; MsCl=methanesulfonyl chloride; Ms20=methanesulfonic anhydride; MTBE=tert-butyl methyl ether; N=Normal; NaHMDS=sodium bis(trimethylsilyl)amide; NBS=N-bromosuccinimide; NCS=N-chlorosuccinimide; NFSI=N-fluorobenzenesulfonamide; [Ni(dtbbpy)(H₂O)₄Cl₂]=tetraaqua[4,4′-bis(1,1-dimethylethyl)-2,2′-bipyridine-κN1,κN1′]nickel (II) chloride; NIS=N-iodosuccinimide; NMP=N-methyl-2-pyrrolidone; NMR=nuclear magnetic resonance; oxone=potassium peroxymonosulfate; Pd(dppf)Cl₂=[1,1′-bis(diphenylphosphino)ferrocene]dichloropalladium (II); Pet. ether=petroleum ether; Ph=phenyl; pin=pinacolato; PMB=4-methoxybenzyl; PMBCI=1-(chloromethyl)-4-methoxybenzene; POCl₃=phosphorus (V) oxide chloride; PPTS=pyridinium p-toluenesulfonate; PyBOP=(benzotriazol-1-yloxy)tripyrrolidinophosphonium hexafluorophosphate; rac=racemic; RP-HPLC=reverse phase HPLC; r.t.=room temperature; RuPhos Pd G3=(2-dicyclohexylphosphino-2′,6′-diisopropoxy-1,1′-biphenyl) [2-(2′-amino-1,1′-biphenyl)]palladium (II) methanesulfonate; RuPhos Pd G4=[dicyclohexyl(2′,6′-diisopropoxy-2-biphenylyl)phosphine-κP](methanesulfonatato-κO) [2′-(methylamino-KN)-2-biphenylyl-KC2]palladium; sat.=saturated; SCN-CO₂Et=ethoxycarbonyl isothiocyanate; selectfluor=1-(chloromethyl)-4-fluoro-1,4-diazabicyclo[2.2.2]octane-1,4-diium ditetrafluoroborate; SEM=(2-methoxyethyl)trimethylsilane; SEMCl=(2-(chloromethoxy)ethyl)trimethylsilane; SFC=supercritical fluid chromatography; SOS=Son of Sevenless; SPhos Pd G3=(2-dicyclohexylphosphino-2′,6′-dimethoxybiphenyl) [2-(2′-amino-1,1′-biphenyl)]palladium (II) methanesulfonate; SPhos Pd G4=(methanesulfonato-κO) [2′-(methylamino)-2-biphenylyl]palladium-dicyclohexyl(2′,6′-dimethoxy-2-biphenylyl)phosphine (1:1); TBAF=tetra-n-butylammonium fluoride; TBDPS=fert-butyldiphenylsilyl; TBDPSCI=tert-butylchlorodiphenylsilyl; TBS=tert-butyl dimethyl silane; (BusP Pd G2=chloro[(tri-tert-butylphosphine)-2-(2-aminobiphenyl)]palladium (II); TEA=Et₃N=triethylamine; TEBAC=benzyltriethylammonium chloride; TES=triethylsilyl; Tf=trifluoromethanesulfonyl; TfO=trifluoromethanesulfonate; TIOH=trifluoromethanesulfonic acid; Tf₂O=trifluoromethanesulfonic anhydride; TFA=trifluoroacetic acid; TFAA=trifluoroacetic anhydride; THF=tetrahydrofuran; THP=tetrahydropyran; TIPS=triisopropypsilyl; TLC=thin layer chromatography; TMEDA=N,N,N′,N′-tetramethyl ethylenediamine; THF=tetrahydrofuran; TLC=thin layer chromatography; TMS=trimethylsilyl; TMSCF₃=trimethyl(trifluoromethyl) silane; TMSCN=Trimethylsilyl cyanide; TR-FRET=time-resolved fluorescence resonance energy transfer; TsOH=p-toluenesulfonic acid=4-methylbenzenesulfonic acid; Tween=polyoxyethylene (20) sorbitan monolaurate; VCD=vibrational circular dichroism; v, v/v=volume, volume to volume; w, w/w=weight, weight to weight, XPhos Pd G2=chloro(2-dicyclohexylphosphino-2′,4′,6′-triisopropyl-1,1′-biphenyl) [2-(2′-amino-1,1′-biphenyl)]palladium (II); um=micrometer.

### Examples

Concentration refers to the removal of the volatile components at reduced pressure (e.g., by rotary evaporation) unless otherwise noted. All temperatures are in degrees Celsius unless otherwise noted. Mass spectra (MS) were measured by electrospray ion-mass spectroscopy (ESI) in positive ion detection mode and m/z refers to the [M+H]⁺ ion unless otherwise noted. ¹H NMR spectra were recorded at 400-600 MHz at ambient temperature unless otherwise noted. Protons reported as 0.5H are due to rotameric signals. RP-HPLC refers to reverse-phase HPLC on C18-functionalized preparative or semi-preparative columns with gradient elution using acetonitrile and water modified with trifluoroacetic acid or ammonium hydroxide as eluents and fractions were lyophilized or concentrated by rotary evaporation unless otherwise noted. Purification by column chromatography on silica gel was accomplished using a flash chromatography system (e.g., ISCO® or Biotage®) and commercial pre-packed silica gel columns with elution using the stated solvent systems. Compounds described herein were synthesized as the racemates unless otherwise noted in the experimental procedures and compound tables. Certain products/intermediates in the examples include indication of “Peak 1” and/or “Peak 2”, which refer to the order of elution of the indicated product/intermediate from the chromatography column (e.g., an SFC column) used to isolate the compound under the specified conditions. Thus, for example, Peak 1 refers to the first eluting compound, e.g., first eluting stereoisomer, under the specified conditions.

SFC and HPLC Columns used in the resolution of stereoisomers are summarized in the following table:

| | Column |
|---|---|
| SFC/HPLC Column | Abbreviation |
| Boston Green ODS (100 mm × 30 mm, 5 um) | Column A |
| Daicel Chiralcel ® OD (250 mm × 30 mm, 10 um) | Column B |
| Perkin Elmer CCO F4 (21 × 250 mm, 5 um) | Column C |
| Chiral Technologies, IB-N (21 mm × 250 mm, 5 um) | Column D |
| Daicel Chiralcel ® OD-H (250 mm × 30 mm, 5 um) | Column E |
| Waters XSelect ® C18 (150 × 30 mm, 5 um) | Column F |
| Daicel Chiralcel ® IG (250 mm × 30 mm, 10 um) | Column G |
| Regin Technologies IZ (21 mm × 250 mm, 5 um) | Column H |
| Phenomenex Biphenyl (21 mm × 250 mm, 5 um) | Column I |
| Daicel Chiralpak ® AS-H (30 × 250 mm, 5 um) | Column J |
| Daicel Chiralpak ® AD (50 × 250 mm, 10 um) | Column K |
| Phenomenex Cellulose (50 mm × 250 mm, 10 um) | Column L |
| Daicel Chiralpak ® AY-H (30 × 250 mm, 10 um) | Column M |
| Daicel Chiralpak ® AS (50 × 250 mm, 10 um) | Column N |
| Phenomenex Cellulose-2 (30 mm × 250 mm, 10 um) | Column O |
| Daicel Chiralpak ® IC (50 × 250 mm, 10 um) | Column P |
| Daicel Chiralcel ® OJ-H (250 mm × 30 mm, 5 um) | Column Q |
| Daicel Chiralpak ® OJ (250 mm × 30 mm, 10 um) | Column R |
| Daicel Chiralpak ® AD-H (250 × 30 mm, 10 um) | Column S |

### Intermediate Syntheses:

### Intermediate 1:(6-methyl-1-(tetrahydro-2H-pyran-2-yl)-5-(trifluoromethyl)-1H-indazol-4-yl)boronic acid (Int-1)

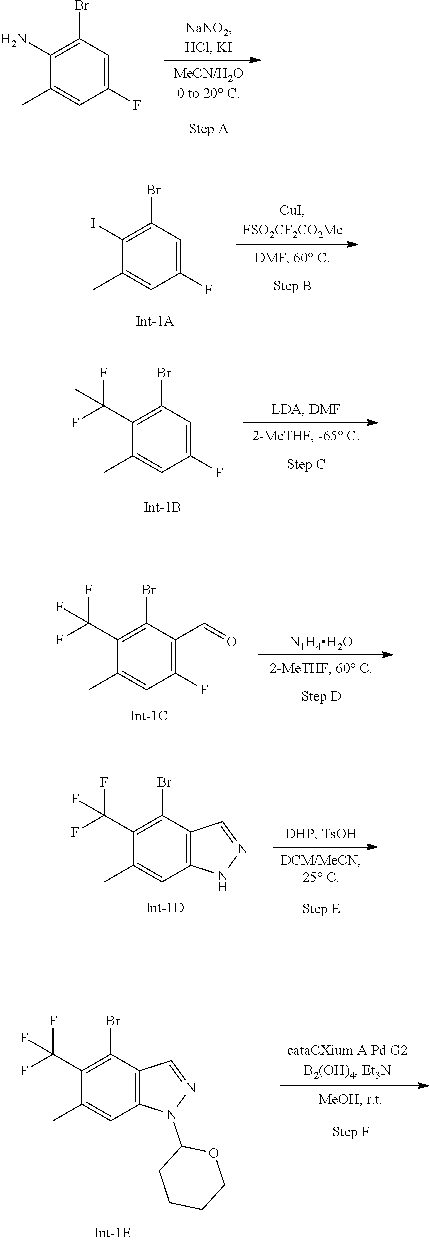

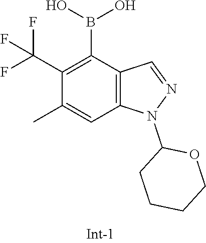 [SMILES: Cc1cc2c(cnn2C2CCCCO2)c(B(O)O)c1C(F)(F)F]

### Step A: 1-bromo-5-fluoro-2-iodo-3-methylbenzene (Int-1A)

2-Bromo-4-fluoro-6-methylaniline (200 g, 0.983 mol) was dissolved in MeCN (800 mL). The resulting mixture was cooled down to 0° C. Concentrated HCl (12 M, 245 mL) was added into the reaction mixture while maintaining the reaction temperature at 0° C. A solution of NaNO₂ (81.1 g, 1.18 mol) in water (400 mL) was added dropwise into the reaction mixture maintaining the reaction temperature at 0° C. The resulting mixture was stirred for 0.5 h at 0° C. Then a solution of KI (195 g, 1.18 mol) in water (400 mL) was added dropwise into the reaction mixture at 0° C. The resulting mixture was warmed up to room temperature and stirred for 12 h at 20° C. This reaction was repeated in one additional batch using the above conditions. The two batches of reactions were combined. The product mixture was adjusted to pH 8-9 by aq. NaOH and the aqueous phase was extracted with EtOAc (2.00 L×2). The organic phase was dried over Na₂SO₄, filtered, and concentrated. The residue obtained was purified by column chromatography (SiO₂, Petroleum ether:Ethyl acetate=1:0 to 0:1) to afford 1-bromo-5-fluoro-2-iodo-3-methylbenzene (Int-1A). ¹H NMR (400 MHz, CDCl₃) δ 7.27-7.22 (m, 1H), 6.95 (dd, J=2.4, 8.8 Hz, 1H), 2.56 (s, 3H).

### Step B: 1-bromo-5-fluoro-3-methyl-2-(trifluoromethyl)benzene (Int-1B)

1-bromo-5-fluoro-2-iodo-3-methylbenzene (Int-1A) (100 g, 0.317 mol) was dissolved in DMF (1.50 L). To this mixture were added CuI (514 g, 2.70 mol) and methyl 2,2-difluoro-2-(fluorosulfonyl)acetate (518 g, 2.70 mol) at 25° C. The reaction mixture was heated and stirred for 12 h at 60° C. This reaction was repeated in 3 additional batches using the above conditions. The four batches of reactions were combined and quenched with water (24 L). The mixture was extracted with petroleum ether (8 L×2). The combined organic layers were washed with brine (4 L×2) and dried over Na₂SO₄. The dried solution was filtered and the filtrate was concentrated in vacuo to afford a crude material containing 1-bromo-5-fluoro-3-methyl-2-(trifluoromethyl)benzene (Int-1B), which was used directly in the next step without further purification.

### Step C: 2-bromo-6-fluoro-4-methyl-3-(trifluoromethyl)benzaldehyde (Int-1C)

1-Bromo-5-fluoro-3-methyl-2-(trifluoromethyl)benzene (Int-1B) (100 g, 0.382 mol) was dissolved in 2-MeTHF (500 mL). The reaction mixture was cooled down to −65° C. A 2 M solution of LDA (213 mL, 426 mmol) was added into the mixture at −65° C. The reaction mixture was stirred for 0.5 h at −65° C. To this mixture was added dropwise DMF (31.2 g, 0.420 mol) at −65° C. The reaction mixture was stirred for 2 h at −65° C. This reaction was repeated in 2 additional batches using the above conditions. The three batches of reactions were combined. The reaction mixture pH was adjusted to 3-4 by using 1 M HCl and the aqueous phase was extracted with 2-MeTHF (500 mL×2). The organic phase was dried over Na₂SO₄, filtered, and concentrated to obtain 2-bromo-6-fluoro-4-methyl-3-(trifluoromethyl)benzaldehyde (Int-1C), which was used directly in the next step without further purification.

### Step D: 4-bromo-6-methyl-5-(trifluoromethyl)-1H-indazole (Int-1D)

2-Bromo-6-fluoro-4-methyl-3-(trifluoromethyl)benzaldehyde (Int-1C) (100 g, 0.351 mol) was dissolved in 2-MeTHF (800 mL). To this mixture was added N₂H₄·H₂O (53.7 g, 1.05 mol) at 25° C. The mixture was heated and stirred for 2 h at 60° C. The product mixture was quenched with water (400 mL) and extracted with EtOAc (200 mL×2). The combined organic layers were washed with brine (200 mL) and dried over Na₂SO₄. The dried solution was filtered and the filtrate was concentrated in vacuo to give the residue. This reaction was repeated in 2 additional batches using the above conditions. The three batches of reactions were combined. The residue obtained was triturated with DCM (100 mL) at 15° C. for 2 h. The solid was collected by filtration to afford 4-bromo-6-methyl-5-(trifluoromethyl)-1H-indazole (Int-1D). ¹H NMR (400 MHz, CDCl₃) δ 10.61-10.20 (m, 1H), 8.20 (d, J=0.8 Hz, 1H), 7.34 (d, J=0.6 Hz, 1H), 2.67-2.63 (m, 3H).

### Step E: 4-bromo-6-methyl-1-(tetrahydro-2H-pyran-2-yl)-5-(trifluoromethyl)-1H-indazole (Int-1E)

4-Bromo-6-methyl-5-(trifluoromethyl)-1H-indazole (Int-1D) (60.0 g, 0.215 mol) was dissolved in DCM (240 mL) and MeCN (240 mL). DHP (21.7 g, 0.258 mol) and TsOH·H₂O (8.2 g, 0.043 mol) were added to the mixture at 20° C. The reaction mixture was stirred for 12 h at 25° C. Water (200 mL) was added to the product mixture. The resulting mixture was extracted with DCM (200 mL×2). The combined organic layers were washed with brine (200 mL) and dried over Na₂SO₄. The dried solution was filtered and the filtrate was concentrated under reduced pressure. The residue obtained was purified by column chromatography (SiO₂, Petroleum ether:Ethyl acetate=1:0 to 0:1) to afford 4-bromo-6-methyl-1-(tetrahydro-2H-pyran-2-yl)-5-(trifluoromethyl)-1H-indazole (Int-1E). ¹H NMR (400 MHz, CDCl₃-d) δ 8.12 (s, 1H), 7.44 (s, 1H), 5.69 (dd, J=3, 9 Hz, 1H), 4.09-3.94 (m, 1H), 3.81-3.69 (m, 1H), 2.69-2.63 (m, 3H), 2.56-2.43 (m, 1H), 2.19-2.14 (m, 1H), 2.12-2.04 (m, 1H), 1.87-1.73 (m, 2H), 1.71-1.63 (m, 1H).

### Step F: (6-methyl-1-(tetrahydro-2H-pyran-2-yl)-5-(trifluoromethyl)-1H-indazol-4-yl)boronic acid (Int-1)

To a solution of 4-bromo-6-methyl-1-(tetrahydro-2H-pyran-2-yl)-5-(trifluoromethyl)-1H-indazole (Int-1E) (1.2 g, 3.3 mmol) in MeOH (15 mL) was added tetrahydroxydiboron (1.185 g, 13.22 mmol), triethylamine (1.38 mL, 9.91 mmol) and cataCXium A Pd G2 (0.110 g, 0.165 mmol). The mixture was stirred at r.t. for 16 h under N₂ atmosphere. The mixture was concentrated in vacuo. The residue was purified by flash silica gel chromatography (0-20% (EtOAc:EtOH=3:1)/petroleum ether) to give (6-methyl-1-(tetrahydro-2H-pyran-2-yl)-5-(trifluoromethyl)-1H-indazol-4-yl)boronic acid (Int-1). MS (ESI) [M+H]⁺: m/z 329.

The compound in the table below was synthesized using a similar procedure as described in the synthesis of Int-1 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | | [M + H\|⁺ |
|---|---|---|---|---|
| Int. | Starting Material | Structure | Compound Name | Found |
| Int-2 | 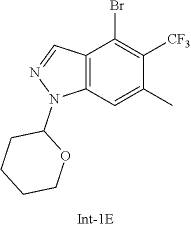 [SMILES: Cc1cc2c(cnn2C2CCCCO2)c(Br)c1C(F)(F)F] | 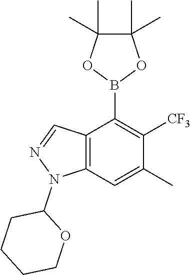 [SMILES: Cc1cc2c(cnn2C2CCCCO2)c(B2OC(C)(C)C(C)(C)O2)c1C(F)(F)F] | 6-methyl-1-(tetrahydro- 2H-pyran-2-yl)-4-(4,4, 5,5-tetramethyl-1,3,2- dioxaborolan-2-yl)-5- (trifluoromethyl)-1H- indazole | 411 |

### Intermediate 3: (7-fluoro-6-methyl-1-(tetrahydro-2H-pyran-2-yl)-5-(trifluoromethyl)-1H-indazol-4-yl)boronic acid (Int-3)

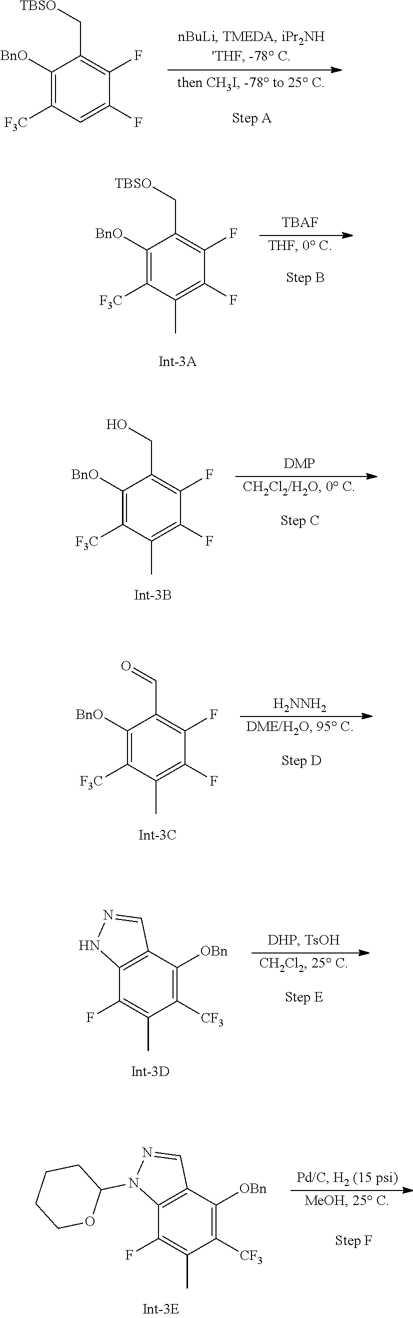

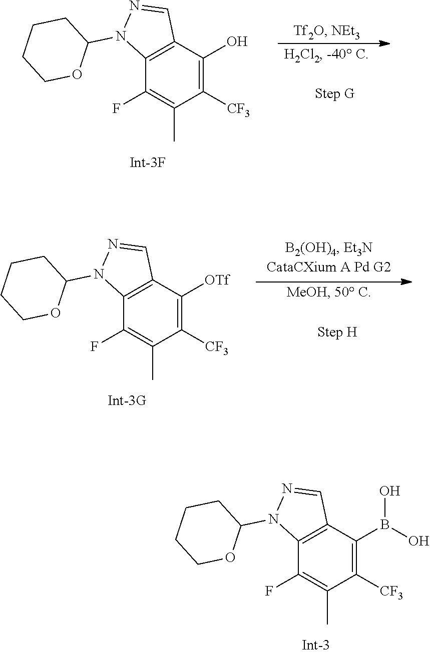

### Step A: ((2-(benzyloxy)-5,6-difluoro-4-methyl-3-(trifluoromethyl)benzyl)oxy) (tert-butyl)dimethylsilane (Int-3A)

To a solution of ((2-(benzyloxy)-5,6-difluoro-3-(trifluoromethyl)benzyl)oxy) (tert-butyl)dimethylsilane (1.5 g, 3.5 mmol) in THF (15 mL) was added TMEDA (1.570 mL, 10.40 mmol) and diisopropylamine (0.035 g, 0.35 mmol) at 25° C., and the resulting mixture was stirred at −78° C. for 10 min. nBuLi (2.50 mL, 6.24 mmol, 2.5 M in hexanes) was added dropwise into the reaction vessel, and the resulting mixture was stirred at −78° C. for 1 h. MeI (0.651 mL, 10.4 mmol) was added into the reaction vessel and the resulting mixture was stirred at 25° C. for 1 h. The product mixture was quenched with sat. aq. NH₄Cl (5 mL) and the product mixture was extracted three times with EtOAc (3×50 mL). The organic layer was dried over sodium sulfate, filtered, and concentrated to dryness to give ((2-(benzyloxy)-5,6-difluoro-4-methyl-3-(trifluoromethyl)benzyl)oxy) (tert-butyl)dimethylsilane (Int-3A), which was used directly in the next step without further purification. ¹H NMR (400 MHz, CDCl₃) δ 7.39-7.34 (m, 2H), 7.32-7.22 (m, 3H), 4.99-4.90 (m, 2H), 4.63 (d, J=2.1 Hz, 2H), 2.31 (m, J=2.8 Hz, 3H), 0.79-0.76 (m, 9H), 0.03-0.02 (m, 6H).

### Step B: (2-(benzyloxy)-5,6-difluoro-4-methyl-3-(trifluoromethyl)phenyl) methanol (Int-3B)

To a solution of ((2-(benzyloxy)-5,6-difluoro-4-methyl-3-(trifluoromethyl)benzyl)oxy) (tert-butyl)dimethylsilane (Int-3A) (1.55 g, 3.47 mmol) in THF (4 mL) was added TBAF (3.47 mL, 3.47 mmol, 1M in THF) at 0° C. and the resulting mixture was stirred at 0° C. for 10 min. The reaction mixture was quenched with sat. aq. NH₄Cl (5 mL). The product mixture was extracted three times with DCM (3×50 mL). The organic layer was dried over sodium sulfate, filtered, and concentrated. The crude mixture was purified by flash silica gel chromatography (20% EtOAc in hexanes) to afford (2-(benzyloxy)-5,6-difluoro-4-methyl-3-(trifluoromethyl)phenyl) methanol (Int-3B). ¹H NMR (400 MHz, CDCl₃) δ 7.41-7.24 (m, 5H), 4.89 (s, 2H), 4.61 (d, J=1.5 Hz, 2H), 2.34 (m, J=2.7 Hz, 3H).

### Step C: 2-(benzyloxy)-5,6-difluoro-4-methyl-3-(trifluoromethyl)benzaldehyde (Int-3C)

To a solution of (2-(benzyloxy)-5,6-difluoro-4-methyl-3-(trifluoromethyl)phenyl) methanol (Int-3B) (800 mg, 2.41 mmol) in DCM (4 mL) was added H₂O (0.043 mL, 2.4 mmol) and DMP (1.53 g, 3.61 mmol) at 0° C. and the resulting mixture was stirred at 0° C. for 3 h. The product mixture was diluted with EtOAc (20 mL) and washed with a 1:1 sat. aq. NaHCO₃: sat. aq. Na₂S₂O₃ solution (10 mL). The organic layer was dried over sodium sulfate, filtered, and concentrated. The crude mixture was purified by flash silica gel chromatography (20% EtOAc in hexanes) to afford 2-(benzyloxy)-5,6-difluoro-4-methyl-3-(trifluoromethyl)benzaldehyde (Int-3C). ¹H NMR (400 MHz, CDCl₃) δ 10.24-9.99 (m, 1H), 7.45-7.27 (m, 5H), 4.99-4.85 (m, 2H), 2.50-2.38 (m, 3H).

### Step D: 4-(benzyloxy)-7-fluoro-6-methyl-5-(trifluoromethyl)-1H-indazole (Int-3D)

To a solution of 2-(benzyloxy)-5,6-difluoro-4-methyl-3-(trifluoromethyl)benzaldehyde (Int-3C) (520 mg, 1.58 mmol) in DME (8 mL) was added hydrazine (0.581 mL, 15.8 mmol, 85% in water) at 25° C., and the resulting mixture was stirred at 95° C. for 1 h. The reaction mixture was cooled to room temperature and diluted with EtOAc (30 mL). The mixture was washed three times with sat. aq. NaHCO₃ (3×5 mL). The organic layer was dried over sodium sulfate, filtered, and concentrated to afford 4-(benzyloxy)-7-fluoro-6-methyl-5-(trifluoromethyl)-1H-indazole (Int-3D). MS (ESI) [M+H]⁺: m/z 325.

### Step E: 4-(benzyloxy)-7-fluoro-6-methyl-1-(tetrahydro-2H-pyran-2-yl)-5-(trifluoromethyl)-1H-indazole (Int-3E)

To a solution of 4-(benzyloxy)-7-fluoro-6-methyl-5-(trifluoromethyl)-1H-indazole (Int-3D) (490 mg, 1.51 mmol) in DCM (5 mL) was added p-toluenesulfonic acid monohydrate (28.7 mg, 0.151 mmol) and DHP (0.27 mL, 3.0 mmol) at 25° C., and the resulting mixture was stirred at 25° C. for 1 h. The reaction mixture was concentrated in vacuo and the residue was purified by flash silica gel chromatography (petroleum ether/EtOAc=5/1) to give 4-(benzyloxy)-7-fluoro-6-methyl-1-(tetrahydro-2H-pyran-2-yl)-5-(trifluoromethyl)-1H-indazole (Int-3E). MS (ESI) [M+H]⁺: m/z 409.

### Step F: 7-fluoro-6-methyl-1-(tetrahydro-2H-pyran-2-yl)-5-(trifluoromethyl)-1H-indazol-4-ol (Int-3F)

To a solution of 4-(benzyloxy)-7-fluoro-6-methyl-1-(tetrahydro-2H-pyran-2-yl)-5-(trifluoromethyl)-1H-indazole (Int-3E) (490 mg, 1.20 mmol) in MeOH (10 mL) was added Pd/C (255 mg, 0.240 mmol, 10% w/w, wet) at 25° C. under N₂, and the mixture was degassed and purged with H₂ for three times and stirred at 25° C. for 24 h under H₂ (15 psi). The product mixture was filtered and concentrated. The residue was purified by flash silica gel chromatography (petroleum ether/EtOAc=5/1) to give 7-fluoro-6-methyl-1-(tetrahydro-2H-pyran-2-yl)-5-(trifluoromethyl)-1H-indazol-4-ol (Int-3F). MS (ESI) [M+H]⁺: m/z 319.

### Step G: 7-fluoro-6-methyl-1-(tetrahydro-2H-pyran-2-yl)-5-(trifluoromethyl)-1H-indazol-4-yl trifluoromethanesulfonate (Int-3G)

To a solution of 7-fluoro-6-methyl-1-(tetrahydro-2H-pyran-2-yl)-5-(trifluoromethyl)-1H-indazol-4-ol (Int-3F) (280 mg, 0.880 mmol) in DCM (5 mL) was added N,N-diisopropylethylamine (0.461 mL, 2.64 mmol) and Tf₂O (0.268 mL, 1.58 mmol) at −40° C. and the resulting mixture was stirred at −40° C. for 10 min. The reaction mixture was warmed to room temperature and concentrated in vacuo and the residue was purified by flash silica gel chromatography (petroleum ether/EtOAc=10/1) to give 7-fluoro-6-methyl-1-(tetrahydro-2H-pyran-2-yl)-5-(trifluoromethyl)-1H-indazol-4-yl trifluoromethanesulfonate (Int-3G). MS (ESI) [M+H]⁺: m/z 451.

### Step H: (7-fluoro-6-methyl-1-(tetrahydro-2H-pyran-2-yl)-5-(trifluoromethyl)-1H-indazol-4-yl)boronic acid (Int-3)

To a solution of 7-fluoro-6-methyl-1-(tetrahydro-2H-pyran-2-yl)-5-(trifluoromethyl)-1H-indazol-4-yl trifluoromethanesulfonate (Int-3G) (50 mg, 0.11 mmol) in MeOH (0.75 mL) was added hypodiboric acid (39.8 mg, 0.444 mmol), cataCXium A Pd G2 (3.71 mg, 5.55 μmol) and triethylamine (0.046 mL, 0.33 mmol) at 20° C. under N₂. The reaction mixture was stirred at 50° C. for 2 h. The reaction mixture was cooled to room temperature and purified by preparative TLC (SiO₂, petroleum ether/EtOAc=3/1) to give (7-fluoro-6-methyl-1-(tetrahydro-2H-pyran-2-yl)-5-(trifluoromethyl)-1H-indazol-4-yl)boronic acid (Int-3). MS (ESI) [M+H]⁺: m/z 347.

### Intermediate 4: (5-(difluoromethyl)-6-methyl-1-(tetrahydro-2H-pyran-2-yl)-1H-indazol-4-yl)boronic acid (Int-4)

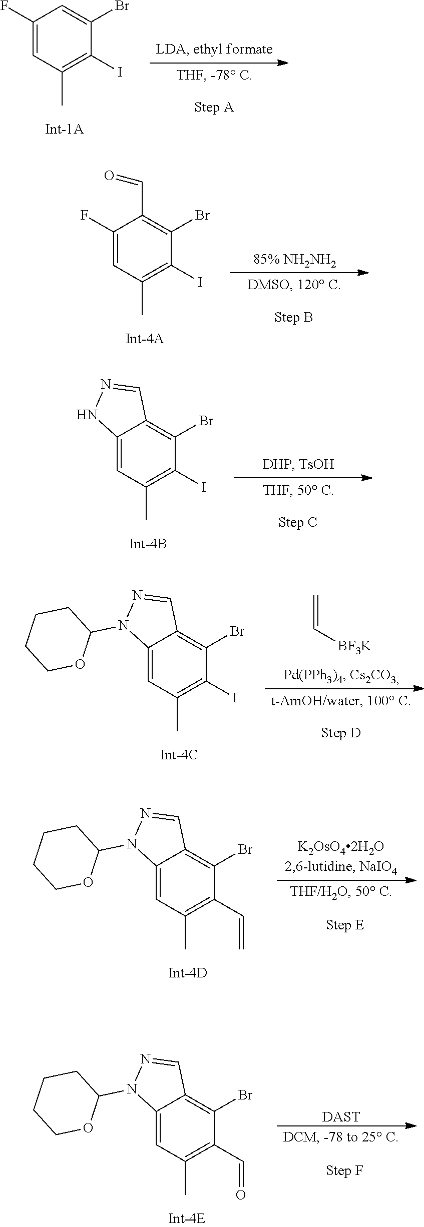

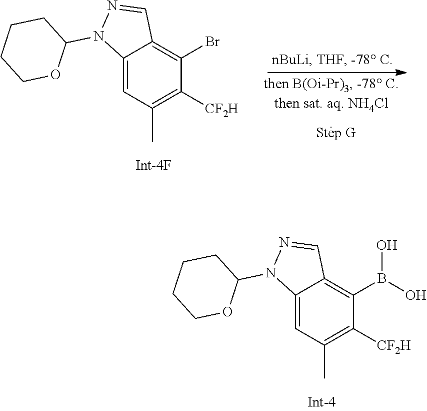 [SMILES: Cc1cc2c(cnn2C2CCCCO2)c(Br)c1C(F)F ; Cc1cc2c(cnn2C2CCCCO2)c(B(O)O)c1C(F)F]

### Step A: 2-bromo-6-fluoro-3-iodo-4-methylbenzaldehyde (Int-4A)

To a solution of diisopropylamine (15.0 ml, 106 mmol) in THF (45 ml) was added n-butyllithium (40 mL, 100 mmol, 2.5 M in hexanes) at −78° C., and the mixture was stirred at −78° C. for 30 min to give LDA. To a solution of 1-bromo-5-fluoro-2-iodo-3-methylbenzene (Int-1A) (24 g, 76 mmol) in THF (45 ml) was added LDA (91 mL, 91 mmol, 1 M in THF) at −78° C. under N₂ atmosphere. The mixture was stirred at −78° C. for 0.5 b, charged with ethyl formate (6.2 g, 84 mmol), and the resulting mixture was stirred at −78° C. for 15 min. The mixture was slowly poured into sat. aq. NH₄Cl (200 mL) and extracted with EtOAc (3×400 mL). The organic layer was dried over sodium sulfate, filtered, and the solvent was concentrated in vacuo. The residue was purified by silica gel chromatography (5% EtOAc in petroleum ether) to give 2-bromo-6-fluoro-3-iodo-4-methylbenzaldehyde (Int-4A). ¹H NMR (400 MHz, CDCl₃) δ 10.16-10.02 (m, 1H), 7.05 (d, J=11.0 Hz, 1H), 2.56 (s, 3H).

### Step B: 4-bromo-5-iodo-6-methyl-1H-indazole (Int-4B)

To a solution of 2-bromo-6-fluoro-3-iodo-4-methylbenzaldehyde (Int-4A) (18.7 g, 54.5 mmol) in DMSO (200 mL) was added hydrazine (19.8 mL, 327 mmol, 85% aqueous solution) at 20° C. under N₂ atmosphere. The reaction mixture was stirred at 120° C. for 12 h. LCMS showed the starting material was consumed and the desired MS was formed. The reaction mixture was poured into ice water (50 mL) and extracted with EtOAc (3×400 mL). The organic layer was washed with sat. aq. NaCl (2×50 mL), dried over sodium sulfate, filtered, and the solvent was concentrated in vacuo to give 4-bromo-5-iodo-6-methyl-1H-indazole (Int-4B), which was used directly in the next step without further purification. MS (ESI): m/z (M+H)⁺ 337, 339.

### Step C: 4-bromo-5-iodo-6-methyl-1-(tetrahydro-2H-pyran-2-yl)-1H-indazole (Int-4C)

To a solution of 4-bromo-5-iodo-6-methyl-1H-indazole (Int-4B) (15.5 g, 46.0 mmol) in THF (200 mL) was added 4-methylbenzenesulfonic acid (1.58 g, 9.20 mmol) and DHP (8.41 mL, 92 mmol) at 20° C., and the mixture was stirred at 50° C. for 12 h. The reaction mixture was concentrated in vacuo, and the residue was purified by silica gel chromatography (0 to 15% EtOAc in petroleum ether). The collected solid was diluted with MeOH (50 mL), and the resulting mixture was stirred for 5 min and then filtered. The solid was collected and dried in vacuum to afford 4-bromo-5-iodo-6-methyl-1-(tetrahydro-2H-pyran-2-yl)-1H-indazole (Int-4C). MS (ESI): m/z (M+H)⁺ 421, 423. ¹H NMR (400 MHz, CDCl₃) δ 7.84 (s, 1H), 7.44-7.35 (m, 1H), 5.58 (dd, J=2.7, 9.1 Hz, 1H), 4.00-3.85 (m, 1H), 3.73-3.59 (m, 1H), 2.67-2.58 (m, 3H), 2.52-2.36 (m, 1H), 2.16-1.93 (m, 2H), 1.76-1.51 (m, 3H).

### Step D: 4-bromo-6-methyl-1-(tetrahydro-2H-pyran-2-yl)-5-vinyl-1H-indazole (Int-4D)

To a solution of 4-bromo-5-iodo-6-methyl-1-(tetrahydro-2H-pyran-2-yl)-1H-indazole (Int-3C) (6.00 g, 14.3 mmol) in t-amyl alcohol (60 mL) and water (20 mL) was added potassium vinyltrifluoroborate (2.481 g, 18.52 mmol), Cs₂CO₃ (13.9 g, 42.7 mmol), and Pd(PPh₃)₄ (0.823 g, 0.712 mmol) at 25° C. The mixture was stirred at 100° C. for 16 h under N₂. The reaction mixture was quenched with H₂O (20 mL) and extracted with EtOAc (3×200 mL). The organic layer was dried over Na₂SO₄, filtered and the solvent was concentrated in vacuo. The residue was purified by silica gel chromatography (0 to 15% EtOAc in petroleum ether) to give 4-bromo-6-methyl-1-(tetrahydro-2H-pyran-2-yl)-5-vinyl-1H-indazole (Int-4D). MS (ESI): m/z (M+H)⁺ 321, 323.

### Step E: 4-bromo-6-methyl-1-(tetrahydro-2H-pyran-2-yl)-1H-indazole-5-carbaldehyde (Int-4E)

To a solution of 4-bromo-6-methyl-1-(tetrahydro-2H-pyran-2-yl)-5-vinyl-1H-indazole (Int-4D) (4.50 g, 12.9 mmol) in THF (50 mL) and water (50 ml) was added sodium periodate (11.03 g, 51.6 mmol), 2,6-dimethylpyridine (2.76 g, 25.8 mmol), and potassium osmate (VI) dihydrate (0.475 g, 1.29 mmol) at 25° C., and the mixture was stirred at 50° C. for 2 h. The reaction mixture was quenched with sat. aq. Na₂SO₃ (20 mL), and the mixture was extracted with EtOAc (3×200 mL). The combined organic layers were washed with brine (20 mL), dried over anhydrous Na₂SO₄, filtered, and concentrated in vacuo. The residue was purified by silica gel chromatography (petroleum ether/EtOAc-5/1) to give 4-bromo-6-methyl-1-(tetrahydro-2H-pyran-2-yl)-1H-indazole-5-carbaldehyde (Int-4E). MS (ESI): m/z (M+H)⁺ 323, 325. ¹H NMR (400 MHz, CDCl₃) δ 10.62-10.44 (m, 1H), 8.09 (s, 1H), 7.30 (s, 1H), 5.62 (dd, J=2.8, 9.1 Hz, 1H), 4.02-3.85 (m, 1H), 3.69 (ddd, J=3.0, 10.0, 11.6 Hz, 1H), 2.66 (d, J=0.7 Hz, 3H), 2.51-2.34 (m, 1H), 2.17-1.92 (m, 2H), 1.78-1.56 (m, 3H).

### Step F: 4-bromo-5-(difluoromethyl)-6-methyl-1-(tetrahydro-2H-pyran-2-yl)-1H-indazole (Int-4F)

To a solution of 4-bromo-6-methyl-1-(tetrahydro-2H-pyran-2-yl)-1H-indazole-5-carbaldehyde (Int-4E) (3.0 g, 9.3 mmol) in DCM (30 mL) was added DAST (6.13 ml, 46.4 mmol) at −78° C. under N₂ atmosphere, and the mixture was stirred at 25° C. for 12 h. The reaction mixture was added dropwise to aq. NaHCO₃ (50 mL), and the mixture was extracted with EtOAc (3×120 mL). The combined organic phase was dried over anhydrous Na₂SO₄, filtered, and concentrated in vacuo. The residue was purified by silica gel chromatography (petroleum ether/EtOAc=5/1) to give 4-bromo-5-(difluoromethyl)-6-methyl-1-(tetrahydro-2H-pyran-2-yl)-1H-indazole (Int-4F). MS (ESI): m/z (M+H)⁺ 345, 347. ¹H NMR (400 MHz, CDCl₃) δ 7.96 (s, 1H), 7.45-7.06 (m, 2H), 5.61 (dd, J=2.8, 9.1 Hz, 1H), 3.97-3.88 (m, 1H), 3.72-3.60 (m, 1H), 2.62 (s, 3H), 2.51-2.38 (m, 1H), 2.14-1.95 (m, 2H), 1.77-1.54 (m, 3H).

### Step G: (5-(difluoromethyl)-6-methyl-1-(tetrahydro-2H-pyran-2-yl)-1H-indazol-4-yl)boronic acid (Int-4)

To a solution of 4-bromo-5-(difluoromethyl)-6-methyl-1-(tetrahydro-2H-pyran-2-yl)-1H-indazole (Int-4F) (0.308 g, 0.892 mmol) in THF (8 ml) was added n-butyllithium (0.725 ml, 1,160 mmol, 1.6 M in hexanes) at −78° C. The resulting mixture was stirred at this temperature for 30 min. Then, triisopropyl borate (618 μL, 2.68 mmol) was added via syringe. The mixture was stirred at −78° C. for 10 min. LCMS showed the reaction was finished, no starting material could be detected, and the product mass was observed. The mixture was quenched with NH₄Cl (aq., 2 mL), extracted with EtOAc (20 mL×3). The organic layer was dried over anhydrous Na₂SO₄, filtered and concentrated in vacuo. (5-(difluoromethyl)-6-methyl-1-(tetrahydro-2H-pyran-2-yl)-1H-indazol-4-yl)boronic acid (Int-4) was obtained and used directly without further purification. MS (ESI): [M+H]⁺ m/z: 311.

The compounds in the table below were synthesized using a similar procedure as described in the synthesis of Int-4 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | | [M + H]⁺ |
|---|---|---|---|---|
| Int. | Starting Material | Structure | Compound Name | Found |
| Int-5 | 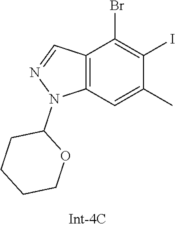 [SMILES: Cc1cc2c(cnn2C2CCCCO2)c(Br)c1I] | 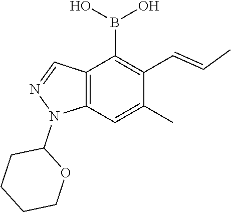 [SMILES: C/C=C/c1c(C)cc2c(cnn2C2CCCCO2)c1B(O)O] | (E)-(6-methyl-5-(prop-1- en-1-yl)-1-(tetrahydro- 2H-pyran-2-yl)-1H- indazol-4-yl)boronic acid | 300 |
| Int-6 | 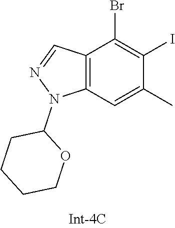 [SMILES: Cc1cc2c(cnn2C2CCCCO2)c(Br)c1I] | 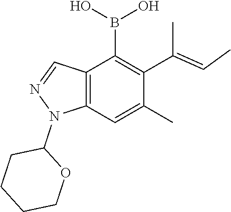 [SMILES: C/C=C(\C)c1c(C)cc2c(cnn2C2CCCCO2)c1B(O)O] | (E)-(5-(but-2-en-2-yl)-6- methyl-1-(tetrahydro-2H- pyran-2-yl)-1H-indazol- 4-yl)boronic acid | 314 |

### Intermediate 7: (5-(2,2-difluorocyclopropyl)-6-methyl-1-(tetrahydro-2H-pyran-2-yl)-1H-indazol-4-yl)boronic acid (Int-7)

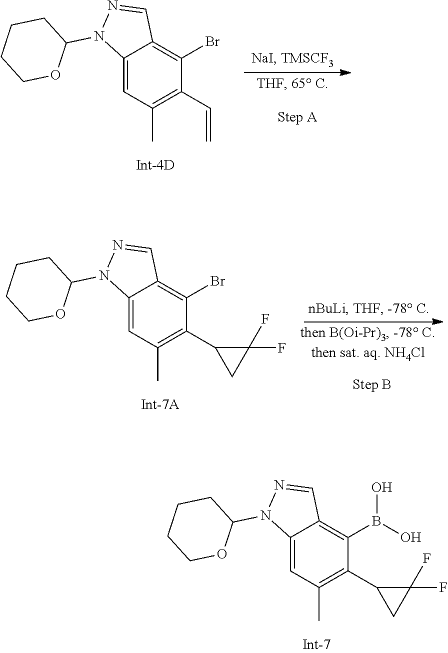 [SMILES: C=Cc1c(C)cc2c(cnn2C2CCCCO2)c1Br ; Cc1cc2c(cnn2C2CCCCO2)c(Br)c1C1CC1(F)F ; Cc1cc2c(cnn2C2CCCCO2)c(B(O)O)c1C1CC1(F)F]

### Step A: 4-bromo-5-(2,2-difluorocyclopropyl)-6-methyl-1-(tetrahydro-2H-pyran-2-yl)-1H-indazole (Int-7A)

4-bromo-6-methyl-1-(tetrahydro-2H-pyran-2-yl)-5-vinyl-1H-indazole (Int-4D) (1.00 g, 3.11 mmol) and sodium iodide (93 mg, 0.62 mmol) were dissolved in THF (3.9 mL). Trimethyl(trifluoromethyl) silane (1.15 mL, 7.78 mmol) was added and the mixture was stirred at 65° C. for 1 h. Additional trimethyl(trifluoromethyl) silane (1.15 mL, 7.78 mmol) was added and the mixture was stirred at 65° C. for 1 h. The reaction was diluted with water and extracted with DCM. The combined organic layers were washed with brine, dried over Na₂SO₄, filtered, and concentrated under reduced pressure. The residue was purified by silica gel chromatography (0-50% (1:9 EtOAc:hexanes) in hexanes) to provide 4-bromo-5-(2,2-difluorocyclopropyl)-6-methyl-1-(tetrahydro-2H-pyran-2-yl)-1H-indazole (Int-7A). MS (ESI) [M+H]⁺: m/z 371, 373.

### Step B: (5-(2,2-difluorocyclopropyl)-6-methyl-1-(tetrahydro-2H-pyran-2-yl)-1H-indazol-4-yl)boronic acid (Int-7)

To a solution of 4-bromo-5-(2,2-difluorocyclopropyl)-6-methyl-1-(tetrahydro-2H-pyran-2-yl)-1H-indazole (Int-7A) (160 mg, 0.431 mmol) in THF (2.16 mL) was added nBuLi in hexanes (350 μL, 0.560 mmol, 1.6 M in hexanes) at −78° C. The result mixture was stirred for 15 min. Triisopropyl borate (298 μL, 1.29 mmol) was added via syringe. The resulting mixture was stirred at −78° C. for 50 min. The mixture was quenched with sat. aq. NH₄Cl and extracted with EtOAc. The organic layer was dried over anhydrous Na₂SO₄, filtered, and concentrated in vacuo to yield (5-(2,2-difluorocyclopropyl)-6-methyl-1-(tetrahydro-2H-pyran-2-yl)-1H-indazol-4-yl)boronic acid (Int-7). (ESI) [M+H]⁺: m/z 337.

### Intermediate 8: (5-chloro-6-methyl-1-(tetrahydro-2H-pyran-2-yl)-3a,7a-dihydro-1H-indazol-4-yl)boronic acid (Int-8)

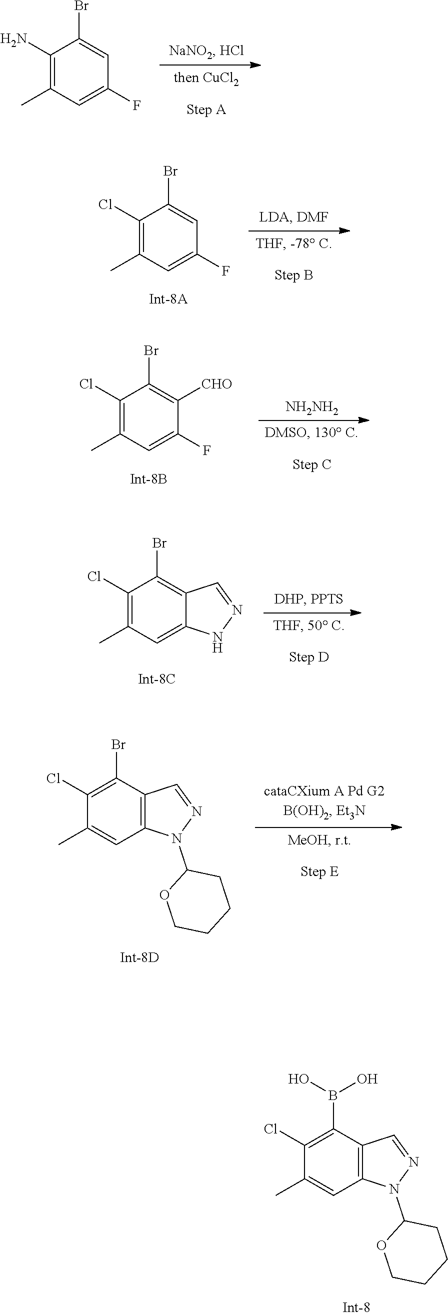

### Step A: 1-bromo-2-chloro-5-fluoro-3-methylbenzene (Int-8A)

Into a 20-L 4-necked round-bottom flask was placed 2-bromo-4-fluoro-6-methylaniline (245 g, 1.2 mol), conc. aq. HCl (2.50 L), and H₂O (2.5 L). The resulting solution was stirred for 1 h at 65° C. This was followed by the addition of a solution of sodium nitrite (99.4 g, 1.44 mol) in H₂O (1 L) dropwise with stirring at 0° C. over 30 min. The resulting solution was stirred for 20 min at 0° C. To this was added a solution of cuprous chloride (178 g, 1.80 mol) in conc. aq. HCl (1.5 L) at 0° C. The resulting solution was allowed to stir for 30 min at 70° C. The resulting solution was extracted with DCM (2×4 L) and the organic layers were combined. The resulting mixture was washed with brine (1×3 L). The mixture was dried over anhydrous sodium sulfate and concentrated under vacuum resulting in 1-bromo-2-chloro-5-fluoro-3-methylbenzene (Int-8A) which was used directly in the next step without further purification.

### Step B: 2-bromo-3-chloro-6-fluoro-4-methylbenzaldehyde (Int-8B)

Into a 10-L 4-necked round-bottom flask was placed 1-bromo-2-chloro-5-fluoro-3-methylbenzene (Int-8A) (200 g, 895 mmol) and THF (2.5 L) and the flask was cooled to −78° C. LDA (843.4 mL, 1.340 mol, 1.6 M in THF) was added dropwise with stirring at −78° C. over 1 h. The resulting solution was stirred for 1 h at −78° C. To this was added N,N-dimethylformamide (98.1 g, 1.34 mol) at −78° C. The resulting solution was allowed to stir for 30 min at −78° C. The reaction was then quenched by the addition of water/ice. The resulting solution was extracted with ethyl acetate (2×3 L) and the organic layers combined. The resulting mixture was washed with brine (1×3 L). The mixture was dried over anhydrous sodium sulfate and concentrated under vacuum resulting in 2-bromo-3-chloro-6-fluoro-4-methylbenzaldehyde (Int-8B) which was used directly in the next step without further purification.

### Step C: 4-bromo-5-chloro-6-methyl-1H-indazole (Int-8C)

Into a 10-L 4-necked round-bottom flask was placed 2-bromo-3-chloro-6-fluoro-4-methylbenzaldehyde (Int-8B) (250 g, 994.1 mmol), DMSO (5 L), and hydrazine (382.3 g, 1.19 mol). The resulting solution was stirred for 2 b at 130° C. The reaction was then quenched by the addition of water/ice and the solids were collected by filtration resulting in 4-bromo-5-chloro-6-methyl-1H-indazole (Int-8C) which was used directly in the next step without further purification.

### Step D: 4-bromo-5-chloro-6-methyl-1-(tetrahydro-2H-pyran-2-yl)-3a, 7a-dihydro-1H-indazole (Int-8D)

Into a 3-L 3-necked round-bottom flask was placed 4-bromo-5-chloro-6-methyl-1H-indazole (Int-8C) (80.00 g, 325.9 mmol), THF (1.2 L), DHP (82.23 g, 977.60 mmol), and PPTS (8.19 g, 32.6 mmol). The resulting solution was stirred for 18 h at 50° C. The reaction was then quenched by the addition of water/ice. The resulting solution was extracted with EtOAc (2×2 L). The organic layers were combined, dried over anhydrous sodium sulfate, and concentrated under vacuum. The residue was purified via silica gel chromatography (1:50 EtOAc/petroleum ether) resulting in 4-bromo-5-chloro-6-methyl-1-(tetrahydro-2H-pyran-2-yl)-3a,7a-dihydro-1H-indazole (Int-8D). MS (ESI): m/z (M+H)⁺ 331, 333. ¹H NMR (300 MHz, CDCl₃): δ 7.95 (d, J=0.9 Hz, 1H), 7.45 (t, J=1.0 Hz, 1H), 5.68 (dd, J=9.0, 2.8 Hz, 1H), 4.00 (d, J=11.7 Hz, 1H), 3.84-3.69 (m, 1H), 2.58 (d, J=0.9 Hz, 3H), 2.55-2.46 (m, 1H), 2.26-2.04 (m, 2H), 1.90-1.63 (m, 3H).

### Step E: (5-chloro-6-methyl-1-(tetrahydro-2H-pyran-2-yl)-3a,7a-dihydro-1H-indazol-4-yl)boronic acid (Int-8)

To a solution of 4-bromo-5-chloro-6-methyl-1-(tetrahydro-2H-pyran-2-yl)-3a, 7a-dihydro-1H-indazole (Int-8D) (750 mg, 2.26 mmol), cataCXium A Pd G2 (91 mg, 0.14 mmol), and tetrahydroxydiboron (811 mg, 9.05 mmol) in MeOH (15 ml) was added triethylamine (0.946 ml, 6.78 mmol). The reaction was stirred at r.t. for 45 min. The reaction was filtered and the filtrate was concentrated. The product was purified by silica gel chromatography (0 to 25% EtOAc in hexane) to provide (5-chloro-6-methyl-1-(tetrahydro-2H-pyran-2-yl)-3a, 7a-dihydro-1H-indazol-4-yl)boronic acid (Int-8). MS (ESI): m/z (M+H)⁺ 297.

### Intermediate 9:5-bromo-4-chloro-6-methyl-1-(tetrahydro-2H-pyran-2-yl)-1H-indazole (Int-9)

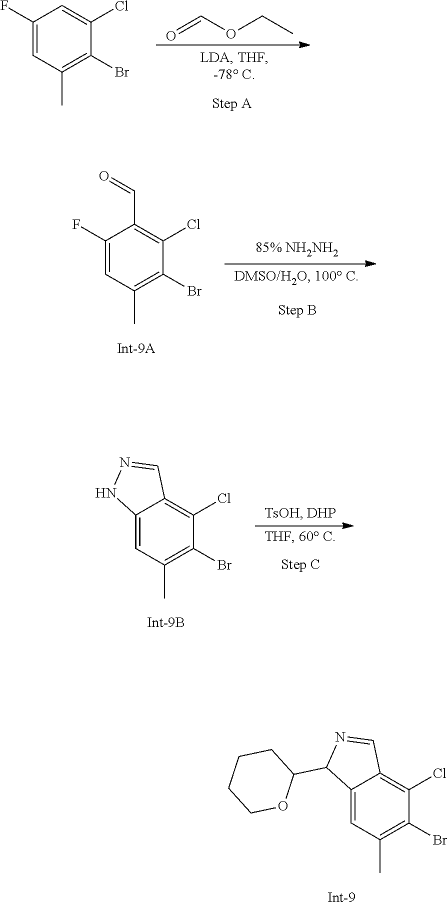

### Step A: 3-bromo-2-chloro-6-fluoro-4-methylbenzaldehyde (Int-9A)

To a solution of 2-bromo-1-chloro-5-fluoro-3-methylbenzene (2.00 g, 8.95 mmol) in THF (20 mL) was added LDA (8.95 mL, 8.95 mmol) (IM in THF) at −78° C. under N₂ atmosphere, and the mixture was stirred at −78° C. for 0.5 h under nitrogen atmosphere. Ethyl formate (0.793 mL, 9.84 mmol) was added to the reaction mixture at −78° C. and the reaction was stirred at −78° C. for 0.5 h. The reaction was quenched with aq. NH₄Cl (10 mL) and the resulting mixture was extracted with EtOAc (3×50 mL). The combined organic phase was dried over anhydrous sodium sulfate, filtered, and concentrated in vacuo to give 3-bromo-2-chloro-6-fluoro-4-methylbenzaldehyde (Int-9A). ¹H NMR (400 MHz, CDCl₃) δ 10.31 (s, 1H), 7.03-6.96 (m, 1H), 2.46 (s, 3H).

### Step B: 5-bromo-4-chloro-6-methyl-1H-indazole (Int-9B)

To a solution of 3-bromo-2-chloro-6-fluoro-4-methylbenzaldehyde (Int-9A) (1.65 g, 6.56 mmol) in DMSO (20 mL) was added hydrazine (2.42 mL, 65.6 mmol, 85% aqueous solution) at 25° C., and the reaction mixture was stirred at 100° C. for 16 h. The mixture was cooled to room temperature and quenched with H₂O (5 mL), and the resulting mixture was diluted with EtOAc (100 mL). The mixture was separated and the organic layer was washed with H₂O (3×5 mL) and brine (5 mL). The organic phase was dried over sodium sulfate, filtered, and the solvent was concentrated in vacuo. The residue was purified by flash silica gel chromatography (petroleum ether/EtOAc=3/1) to give 5-bromo-4-chloro-6-methyl-1H-indazole (Int-9B). MS (ESI) [M+H]⁺: m/z 245, 247.

### Step C: 5-bromo-4-chloro-6-methyl-1-(tetrahydro-2H-pyran-2-yl)-1H-indazole (Int-9)

To a solution of 5-bromo-4-chloro-6-methyl-1H-indazole (Int-9B) (790 mg, 3.22 mmol) in THF (8 mL) was added 3,4-dihydro-2H-pyran (0.883 mL, 9.65 mmol) and p-toluenesulfonic acid (55.4 mg, 0.322 mmol) at 25° C., and the mixture was stirred at 60° C. for 6 h. The reaction mixture was concentrated in vacuo and the residue was purified by flash silica gel chromatography (petroleum ether/EtOAc=10/1) to give 5-bromo-4-chloro-6-methyl-1-(tetrahydro-2H-pyran-2-yl)-1H-indazole (Int-9). MS (ESI) [M+H]⁺: m/z 329, 331.

The compound in the table below was synthesized using a similar procedure as described in the synthesis of Int-9 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| Int. | Starting Material | Structure | Compound Name | ¹H NMR |
|---|---|---|---|---|
| Int-10 | 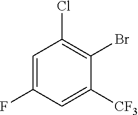 [SMILES: Fc1cc(Cl)c(Br)c(C(F)(F)F)c1] | 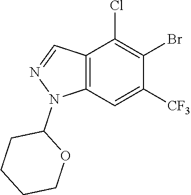 [SMILES: FC(F)(F)c1cc2c(cnn2C2CCCCO2)c(Cl)c1Br] | 5-bromo-4-chloro-1- (tetrahydro-2H-pyran- 2-yl)-6-(trifluorometh- yl)-1H-indazole | (400 MHZ, CDCl₃) δ 8.13 (s, 1 H), 7.97 (s, 1 H), 5.77 (dd, J = 9.20, 1 H), 3.97-4.00 (m, 1 H), 3.75- 3.80 (m, 1 H), 2.48-2.50 (m, 1 H), 2.11-2.14 (m, 2 H), 1.71-1.79 (m, 3 H). |

### Intermediate 11: (5-(prop-1-en-2-yl)-1-(tetrahydro-2H-pyran-2-yl)-6-(trifluoromethyl)-1H-indazol-4-yl)boronic acid (Int-11)

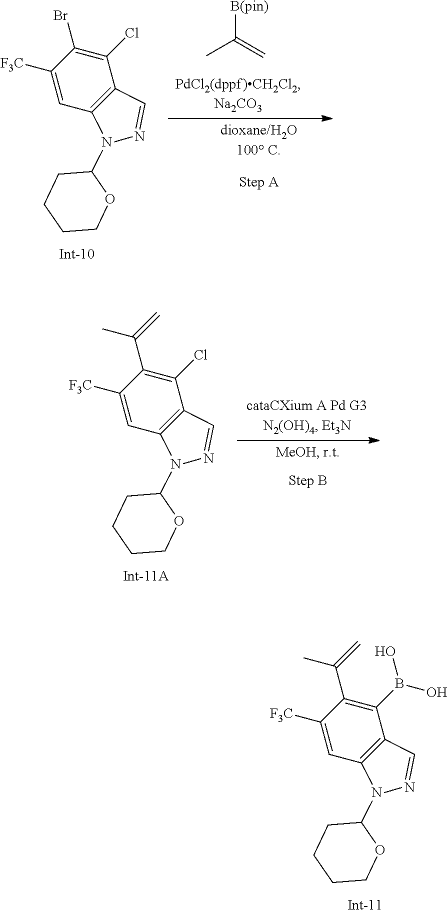

### Step A: 4-chloro-5-(prop-1-en-2-yl)-1-(tetrahydro-2H-pyran-2-yl)-6-(trifluoromethyl)-1H-indazole (Int-11A)

Na₂CO₃ (39.1 mL, 39.1 mmol, 1M in H₂O) was added to a solution of 5-bromo-4-chloro-1-(tetrahydro-2H-pyran-2-yl)-6-(trifluoromethyl)-1H-indazole (Int-10) (3.00 g, 7.82 mmol), 2-isopropenyl-4,4,5,5-tetramethyl-1,3,2-dioxaborolane (1.58 g, 9.39 mmol), and Pd(dppf)Cl₂·CH₂Cl₂ (95.8 mg, 1.17 mmol) in dioxane (100 mL) at room temperature. The reaction mixture was heated to 100° C. and stirred for 13 h. The reaction was quenched by the addition of H₂O and extracted with EtOAc. The combined organics were washed with brine, dried over Na₂SO₄, filtered, and concentrated in vacuo. The residue was purified by flash silica gel chromatography (0 to 7%, EtOAc in hexane) to afford 4-chloro-5-(prop-1-en-2-yl)-1-(tetrahydro-2H-pyran-2-yl)-6-(trifluoromethyl)-1H-indazole (Int-11A). ESI-MS m/z [M+H]⁺ 345.

### Step B: (5-(prop-1-en-2-yl)-1-(tetrahydro-2H-pyran-2-yl)-6-(trifluoromethyl)-1H-indazol-4-yl)boronic acid (Int-11)

Triethylamine (2.93 mL, 21.0 mmol) was added to a solution of 4-chloro-5-(prop-1-en-2-yl)-1-(tetrahydro-2H-pyran-2-yl)-6-(trifluoromethyl)-1H-indazole (Int-11A) (1.81 g, 5.25 mmol), tetrahydroxydiboron (1.41 g, 15.8 mmol), and cataCXium A Pd G3 (382 mg, 0.525 mmol) in MeOH (52.5 mL) at room temperature. After stirring the mixture at room temperature for 13 h, the reaction was quenched by the addition of H₂O. The reaction mixture was extracted with CHCl₃, washed with brine, dried over Na₂SO₄, filtered, and concentrated in vacuo. The residue was purified by flash silica gel chromatography (17 to 38% EtOAc in hexane) to afford (5-(prop-1-en-2-yl)-1-(tetrahydro-2H-pyran-2-yl)-6-(trifluoromethyl)-1H-indazol-4-yl)boronic acid (Int-11). ESI-MS m/z [M+H]⁺ 355. ¹H-NMR (400 MHz, CDCl₃) δ 8.51 (s, 1H), 8.06 (s, 1H), 5.78 (dd, J=9.0, 2.5 Hz, 1H), 5.64-5.57 (m, 3H), 5.15 (s, 1H), 4.07-3.98 (m, 1H), 3.83-3.70 (m, 1H), 2.64-2.48 (m, 1H), 2.23-2.06 (m, 5H), 1.86-1.64 (m, 3H).

### Intermediate 12: (5-(chlorodifluoromethoxy)-6-methyl-1-(tetrahydro-2H-pyran-2-yl)-1H-indazol-4-yl)boronic acid (Int-12)

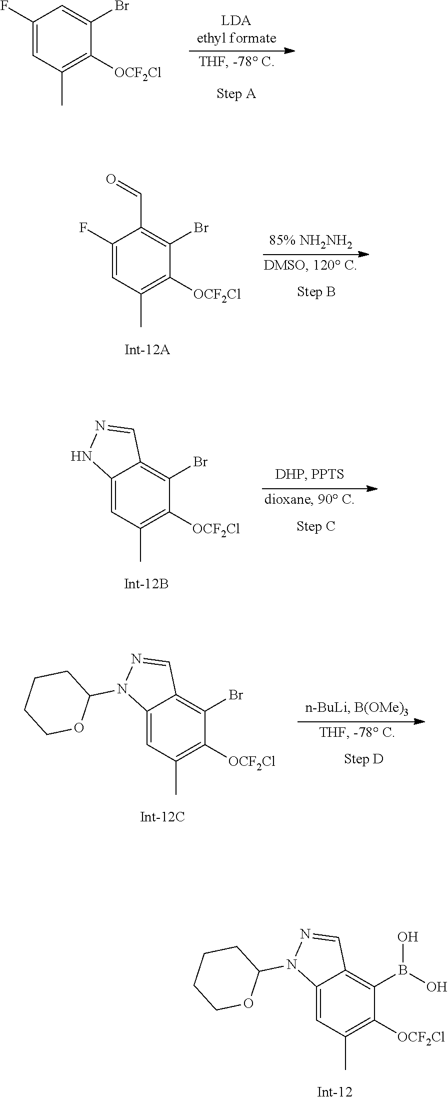

### Step A: 2-bromo-3-(chlorodifluoromethoxy)-6-fluoro-4-methylbenzaldehyde (Int-12A)

To a solution of 1-bromo-2-(chlorodifluoromethoxy)-5-fluoro-3-methylbenzene (1.00 g, 3.45 mmol) in THF (10 mL) was added lithium diisopropylamide (4.15 mL, 4.15 mmol, 1 M in THF) at −78° C. under N₂ atmosphere. The mixture was stirred at −78° C. for 1 h. Then to the reaction mixture was added ethyl formate (0.281 g, 3.80 mmol) at −78° C. and the resulting mixture was stirred at −78° C. for 0.5 h. The mixture was diluted with aq. NH₄Cl (5 mL) and extracted with EtOAc (3×15 mL), dried over sodium sulfate, filtered and the solvent was evaporated under reduced pressure to give a crude product. The crude product was purified by silica gel chromatography (0 to 25% EtOAc in petroleum ether) to give 2-bromo-3-(chlorodifluoromethoxy)-6-fluoro-4-methylbenzaldehyde (Int-12A). ¹H NMR (400 MHz, CDCl₃) δ:10.28-10.37 (m, 1H), 7.10 (d, J=10.4 Hz, 1H), 2.48 (s, 3H).

### Step B: 4-bromo-5-(chlorodifluoromethoxy)-6-methyl-1H-indazole (Int-12B)

To a solution of 2-bromo-3-(chlorodifluoromethoxy)-6-fluoro-4-methylbenzaldehyde (Int-12A) (0.900 g, 2.83 mmol) in DMSO (9 mL) was added hydrazine (0.534 g, 14.2 mmol, 85% in water) at 20° C. under N₂ atmosphere. The mixture was stirred at 120° C. for 12 h. LCMS showed the reaction was completed. The mixture was diluted with water (10 mL) and extracted with EtOAc (3×30 mL). The organic layer was washed with brine (3×10 mL), dried over sodium sulfate, filtered, and the solvent was evaporated under reduced pressure to give a crude product. The crude product was purified by silica gel chromatography (0 to 10% EtOAc in petroleum ether) to give 4-bromo-5-(chlorodifluoromethoxy)-6-methyl-1H-indazole (Int-12B). MS (ESI) [M+H]⁺: m/z 311, 313.

### Step C: 4-bromo-5-(chlorodifluoromethoxy)-6-methyl-1-(tetrahydro-2H-pyran-2-yl)-1H-indazole (Int-12C)

To a solution of 4-bromo-5-(chlorodifluoromethoxy)-6-methyl-1H-indazole (Int-12B) (630 mg, 2.02 mmol) in dioxane (7 mL) was added pyridinium p-toluenesulfonate (102 mg, 0.404 mmol) and 3,4-dihydro-2H-pyran (340 mg, 4.04 mmol) at 20° C. The mixture was stirred at 90° C. for 12 h. LCMS showed the reaction was completed. The mixture was diluted with water (4 mL) and extracted with EtOAc (3×10 mL), dried over sodium sulfate, filtered, and the solvent was evaporated under reduced pressure to give a crude product. The crude product was purified by silica gel chromatography (0 to 20% EtOAc in petroleum ether) to give 4-bromo-5-(chlorodifluoromethoxy)-6-methyl-1-(tetrahydro-2H-pyran-2-yl)-1H-indazole (Int-12C). MS (ESI) [M+H]⁺: m/z 395, 397.

### Step D: (5-(chlorodifluoromethoxy)-6-methyl-1-(tetrahydro-2H-pyran-2-yl)-1H-indazol-4-yl)boronic acid (Int-12)

To a solution of 4-bromo-5-(chlorodifluoromethoxy)-6-methyl-1-(tetrahydro-2H-pyran-2-yl)-1H-indazole (Int-12C) (550 mg, 1.39 mmol) in THF (10 mL) was added n-butyllithium (0.834 mL, 2.09 mmol, 2.5 M in hexane) at −78° C. under N₂ atmosphere. The reaction was stirred at −78° C. for 10 min. Trimethyl borate (0.311 mL, 2.78 mmol) in THF (1 mL) was added dropwise, and the reaction mixture was stirred at −78° C. for 1 h. LCMS showed the starting material was consumed and desired MS was formed. The mixture was quenched with sat. aq. NH₄Cl (5 mL) at −78° C. The mixture was extracted with EtOAc (20 mL×2) and washed with brine (10 mL), dried over Na₂SO₄, and concentrated in vacuum. The residue was purified by silica gel chromatography (0 to 20% EtOAc in petroleum ether then 10% (EtOAc/EtOH=3:1) in petroleum ether) to give (5-(chlorodifluoromethoxy)-6-methyl-1-(tetrahydro-2H-pyran-2-yl)-1H-indazol-4-yl)boronic acid (Int-12). MS (ESI) [M+H]⁺: m/z 361.

### Intermediate 13: (5-ethoxy-6-methyl-1-(tetrahydro-2H-pyran-2-yl)-1H-indazol-4-yl)boronic acid (Int-13)

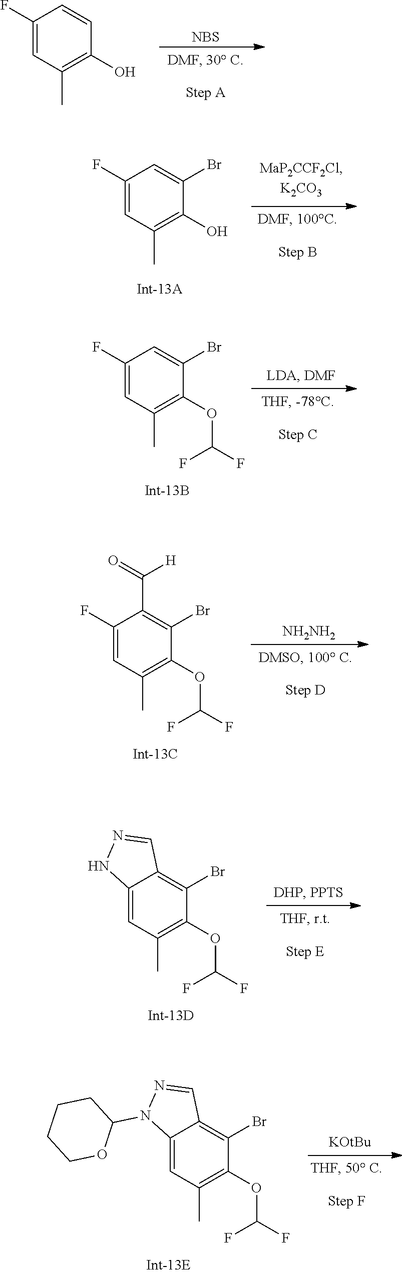

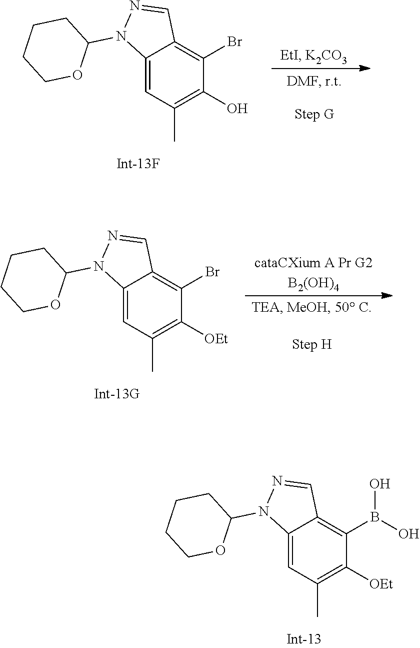

### Step A: 2-bromo-4-fluoro-6-methylphenol (Int-13A)

To a stirred solution of 4-fluoro-2-methylphenol (7.65 g, 60.7 mmol) in DMF (80 mL) was added NBS (10.8 g, 60.7 mmol), and the mixture was stirred at 30° C. for 18 h. The reaction mixture was quenched with water (80 mL) and extracted with EtOAc (3×80 mL). The combined organic layer was dried over Na₂SO₄, filtered, and the solvent was evaporated under reduced pressure. The residue was purified by silica gel chromatography (0 to 20% EtOAc in petroleum ether) to give 2-bromo-4-fluoro-6-methylphenol (Int-13A). ¹H NMR (400 MHz, CDCl₃) δ 7.04 (dd, J=2.7, 7.4 Hz, 1H), 6.83 (dd, J=2.9, 8.8 Hz, 1H), 2.28 (s, 3H).

### Step B: 1-bromo-2-(difluoromethoxy)-5-fluoro-3-methylbenzene (Int-13B)

To a mixture of 2-bromo-4-fluoro-6-methylphenol (Int-13A) (4.00 g, 19.5 mmol) in DMF (50 mL) was added sodium chlorodifluoroacetate (4.46 g, 29.3 mmol) and K₂CO₃ (8.09 g, 58.5 mmol), and the mixture was stirred at 100° C. for 15 h. The reaction mixture was quenched with water (30 mL), then the mixture was extracted with EtOAc (100 mL×3), the combined organic layers were washed with brine (50 mL), dried over anhydrous Na₂SO₄, filtered, and concentrated in vacuo. The crude product was purified by silica gel chromatography (0 to 10% EtOAc in petroleum ether) to give 1-bromo-2-(difluoromethoxy)-5-fluoro-3-methylbenzene (Int-13B). ¹H NMR (400 MHz, CDCl₃) δ 7.18 (dd, J=3.1, 7.4 Hz, 1H), 6.92 (dd, J=2.7, 8.2 Hz, 1H), 6.48 (t, J=74.8, Hz, 1H), 2.36 (s, 3H).

### Step C: 2-bromo-3-(difluoromethoxy)-6-fluoro-4-methylbenzaldehyde (Int-13C)

To a solution of diisopropylamine (2.5 g, 25 mmol) in THF (50 mL) was added n-BuLi (7.80 mL, 19.5 mmol, 2.5 M in hexanes) slowly at 0° C., the mixture was stirred at 0° C. for 30 min, and then cooled to −78° C. to give LDA. Then a solution of 1-bromo-2-(difluoromethoxy)-5-fluoro-3-methylbenzene (Int-13B) (5.0 g, 20 mmol) in THF (5.0 mL) was added to the above mixture, and the resulting mixture was stirred at −78° C. for 40 min. Then DMF (5 mL) was added and the mixture was stirred at −78° C. for another 10 min. The reaction mixture was quenched with water (2×5 mL) and extracted with EtOAc (3×60 mL). The combined organic layer was washed with brine (30 mL), dried over Na₂SO₄, filtered, and the solvent was evaporated under reduced pressure. The crude product was purified by silica gel chromatography (0 to 30% ethyl acetate in petroleum ether) to give 2-bromo-3-(difluoromethoxy)-6-fluoro-4-methylbenzaldehyde (Int-13C). ¹H NMR (400 MHz, CDCl₃) δ 10.30 (s, 1H), 7.07 (d, J=10.2 Hz, 1H), 6.53 (t, J=74.4 Hz, 1H), 2.45 (s, 3H).

### Step D: 4-bromo-S-(difluoromethoxy)-6-methyl-1H-indazole (Int-13D)

To a solution of 2-bromo-3-(difluoromethoxy)-6-fluoro-4-methylbenzaldehyde (Int-13C) (800 mg, 2.83 mmol) in DMSO (8.00 mL) was added hydrazine hydrate (832 mg, 14.1 mmol, 85% in H₂O), and the mixture was stirred at 100° C. for 15 h. The mixture was cooled and diluted with water (20 mL) and extracted with EtOAc (3×60 mL). The combined organic layer was dried over Na₂SO₄, filtered, and the solvent was evaporated under reduced pressure to give a crude product. The crude product was purified by silica gel chromatography (0 to 30% EtOAc in petroleum ether) to give 4-bromo-5-(difluoromethoxy)-6-methyl-1H-indazole (Int-13D). MS (ESI): m/z [M+H]⁺ 277.

### Step E: 4-bromo-5-(difluoromethoxy)-6-methyl-1-(tetrahydro-2H-pyran-2-yl)-1H-indazole (Int-13E)

To a solution of 4-bromo-5-(difluoromethoxy)-6-methyl-1H-indazole (Int-13D) (650 mg, 2.35 mmol) in THF (10.0 mL) was added pyridinium p-toluenesulfonate (59.0 mg, 0.235 mmol) and 3,4-dihydro-2H-pyran (987 mg, 11.7 mmol). The mixture was stirred at 25° C. for 15 h. The mixture was diluted with water (20 mL), extracted with EtOAc (3×30 mL), the combined organic layer was dried over with Na₂SO₄, filtered, and the solvent was evaporated under reduced pressure. The crude product was purified by silica gel chromatography (0 to 30% ethyl acetate in petroleum ether) to give 4-bromo-5-(difluoromethoxy)-6-methyl-1-(tetrahydro-2H-pyran-2-yl)-1H-indazole (Int-13E). MS (ESI): m/z [M+H]⁺ 361.

### Step F: 4-bromo-6-methyl-1-(tetrahydro-2H-pyran-2-yl)-1H-indazol-5-ol (Int-13E)

A mixture of 4-bromo-5-(difluoromethoxy)-6-methyl-1-(tetrahydro-2H-pyran-2-yl)-1H-indazole (Int-13E) (1.5 g, 4.2 mmol) and potassium tert-butoxide (2.33 g, 21 mmol) in THF (24 mL) were stirred at room temperature for 2 hr then heated at 50° C. for 4 hr. The reaction mixture was cooled to room temperature, diluted with EtOAc, acidified to pH 4-5 with 1N HCl, and extracted with EtOAc. The combined extracts were washed with brine solution and concentrated. The product was purified by silica gel chromatography (0-100% of EtOAc in hexane) to provide 4-bromo-6-methyl-1-(tetrahydro-2H-pyran-2-yl)-1H-indazol-5-ol (Int-13F). MS (ESI): m/z [M+H]⁺ 311.

### Step G: 4-bromo-5-ethoxy-6-methyl-1-(tetrahydro-2H-pyran-2-yl)-1H-indazole (Int-13G)

A mixture of 4-bromo-6-methyl-1-(tetrahydro-2H-pyran-2-yl)-1H-indazol-5-ol (Int-13F) (300 mg, 0.964 mmol), potassium carbonate (150 mg, 1.1 mmol), and ethyl iodide (0.093 mL, 1.1 mmol) in DMF (6.0 mL) was stirred at r.t. for 4 hr. After 4 hr, the reaction was quenched with water and extracted with EtOAc. The combined extracts were washed with water and brine solution. The organic phase was concentrated to provide 4-bromo-5-ethoxy-6-methyl-1-(tetrahydro-2H-pyran-2-yl)-1H-indazole (Int-13G) which was used without further purification. MS (ESI): m/z [M+H]⁺ 339.

### Step H: (5-ethoxy-6-methyl-1-(tetrahydro-2H-pyran-2-yl)-1H-indazol-4-yl) boronic acid (Int-13)

A mixture of 4-bromo-5-ethoxy-6-methyl-1-(tetrahydro-2H-pyran-2-yl)-1H-indazole (Int-13G) (300 mg, 0.9 mmol)), cataCXium A Pd G2 (36 mg, 0.053 mmol)), tetrahydroxydiboron (242 mg, 2.7 mmol), and triethylamine (0.37 mL, 2.7 mmol) in MeOH (7 ml) were stirred at 50° C. for 2 hr. The reaction mixture was cooled to room temperature, filtered and concentrated. The crude material was purified by silica gel chromatography (0-100% EtOAc in hexane) to provide (5-ethoxy-6-methyl-1-(tetrahydro-2H-pyran-2-yl)-1H-indazol-4-yl)boronic acid (Int-13). MS (ESI): m/z [M+H]⁺ 305.

### Intermediate 14: (6-methyl-1-(tetrahydro-2H-pyran-2-yl)-5-(trifluoromethyl)thio)-1H-indazol-4-yl)boronic acid (Int-14)

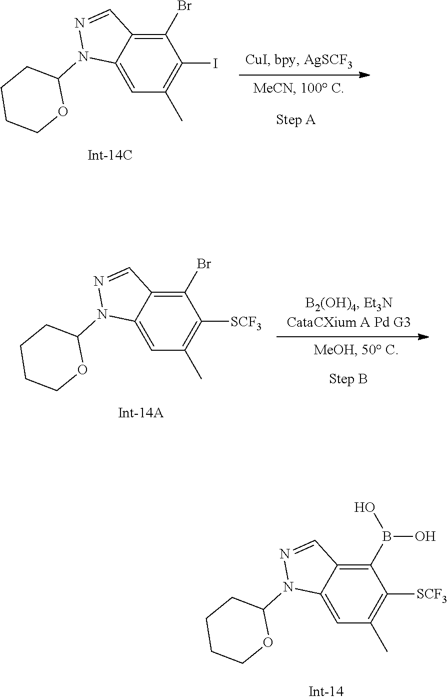

### Step A: 4-bromo-6-methyl-1-(tetrahydro-2H-pyran-2-yl)-5-((trifluoromethyl)thio)-1H-indazole (Int-14A)

To a solution of 4-bromo-5-iodo-6-methyl-1-(tetrahydro-2H-pyran-2-yl)-1H-indazole (Int-4C) (400 mg, 0.950 mmol) in MeCN (20 mL) was added silver (I) trifluoromethanethiolate (992 mg, 4.75 mmol), copper (I) iodide (543 mg, 2.85 mmol), and 2,2′-bipyridine (445 mg, 2.85 mmol) at 25° C., and the mixture was stirred at 100° C. for 48 h under N₂. The mixture was concentrated in vacuo and the residue was partitioned between EtOAc (30 mL) and brine (18 mL). The layers were separated and the aqueous phase was washed with EtOAc (2×30 mL). The organic layer was dried over sodium sulfate, filtered, and concentrated under reduced pressure. The residue was purified by preparative reverse-phase HPLC (MeCN/H₂O w/NH₄HCO₃ modifier). The mixture was purified a second time by preparative SFC (Column G, 45% EtOH w/0.1% NH₄OH) to give 4-bromo-6-methyl-1-(tetrahydro-2H-pyran-2-yl)-5-((trifluoromethyl)thio)-1H-indazole (Int-14A). MS (ESI) [M+H]⁺: m/z 395, 397.

### Step B: (6-methyl-1-(tetrahydro-2H-pyran-2-yl)-5-((trifluoromethyl)thio)-1H-indazol-4-yl)boronic acid (Int-14)

To a solution of 4-bromo-6-methyl-1-(tetrahydro-2H-pyran-2-yl)-5-((trifluoromethyl)thio)-1H-indazole (Int-14A) (167 mg, 0.423 mmol) in MeOH (2 mL) was added hypodiboric acid (152 mg, 1.69 mmol), triethylamine (0.177 mL, 1.27 mmol) and cataCXium A Pd G3 (14.13 mg, 0.0210 mmol) at 25° C. under N₂, and the reaction mixture was stirred at 50° C. for 1 h. The reaction mixture was cooled to room temperature and purified by preparative TLC (SiO₂, petroleum ether/EtOAc=1/1) to give (6-methyl-1-(tetrahydro-2H-pyran-2-yl)-5-((trifluoromethyl)thio)-1H-indazol-4-yl)boronic acid (Int-14). MS (ESI) [M+H]⁺: m/z 361.

### Intermediate 15: (6-chloro-5-(difluoromethyl)-1-(tetrahydro-2H-pyran-2-yl)-1H-indazol-4-yl)boronic acid (Int-15)

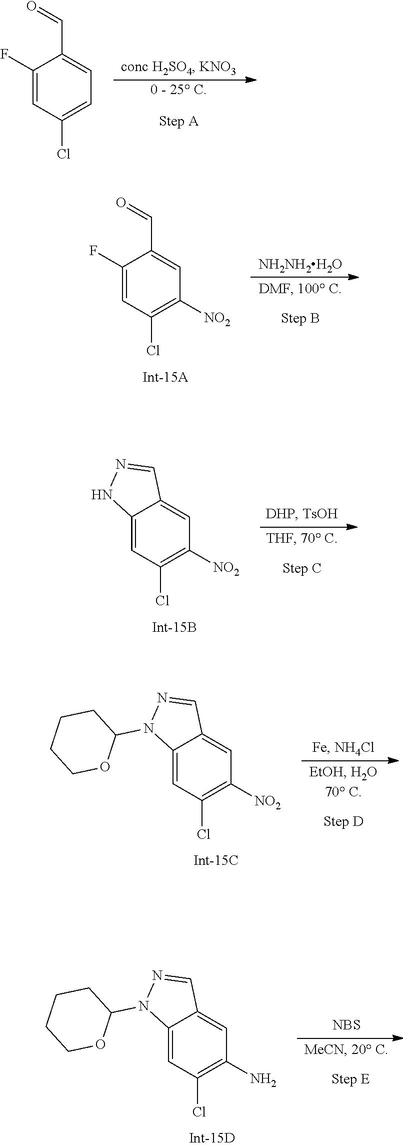

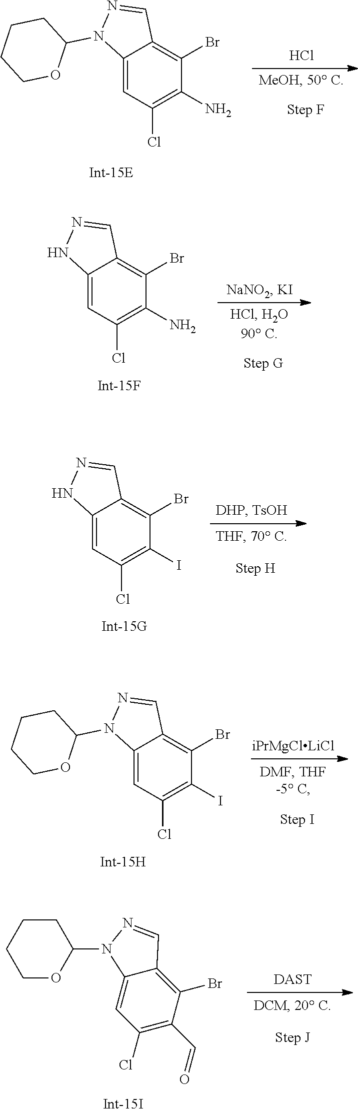

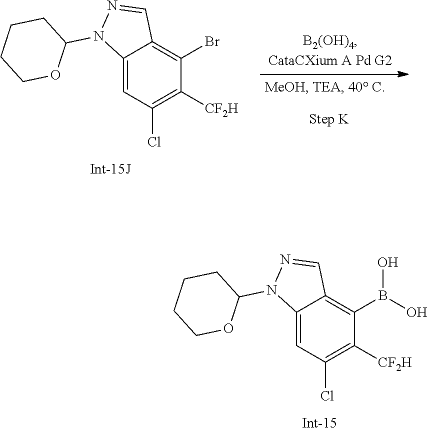 [SMILES: FC(F)c1c(Cl)cc2c(cnn2C2CCCCO2)c1Br ; OB(O)c1c(C(F)F)c(Cl)cc2c1cnn2C1CCCCO1]

### Step A: 4-chloro-2-fluoro-5-nitrobenzaldehyde (Int-15A)

To a solution of 4-chloro-2-fluorobenzaldehyde (58 g, 370 mmol) in conc. H₂SO₄ (500 mL) was added potassium nitrate (47 g, 470 mmol) at 0° C. Then the reaction was stirred at 25° C. for 1 h. The reaction mixture was quenched with ice water (2 L), filtered, and the solid was washed with water (2×500 mL), dried in vacuo to give 4-chloro-2-fluoro-5-nitrobenzaldehyde (Int-15A). ¹H NMR (400 MHz, CDCl₃) δ 10.32 (s, 1H), 8.47 (d, J=6.6 Hz, 1H), 7.48 (d, J=9.2 Hz, 1H).

### Step B: 6-chloro-5-nitro-1H-indazole (Int-15B)

To a solution of 4-chloro-2-fluoro-5-nitrobenzaldehyde (Int-15A) (67.0 g, 329 mmol) in DMF (1.00 L) were added hydrazine hydrate (161 mL, 2.82 mol) at 25° C. under N₂ atmosphere. The reaction mixture was stirred at 100° C. for 15 h. The reaction mixture was cooled and quenched with ice water (2 L). The mixture was filtered and the filtered cake was washed with water (2×300 mL), dried in vacuum to give 6-chloro-5-nitro-1H-indazole (Int-15B). MS (ESI) [M+H]⁺: m/z 198. ¹H NMR (400 MHz, DMSO-d₆) δ 8.44 (s, 1H), 8.25 (s, 1H), 7.70 (s, 1H).

### Step C: 6-chloro-5-nitro-1-(tetrahydro-2H-pyran-2-yl)-1H-indazole (Int-15C)

To a solution of 6-chloro-5-nitro-1H-indazole (Int-15B) (92 g, 0.47 mol) in THF (1.0 L) were added 3,4-dihydro-2H-pyran (85 mL, 0.93 mol) and p-toluenesulfonic acid (8.0 g, 47 mmol) at 25° C. under N₂ atmosphere. The reaction mixture was stirred at 70° C. for 5 h. The reaction mixture was cooled and evaporated under reduced pressure to give a crude product. The crude product was purified by flash silica gel chromatography (20% ethyl acetate in petroleum ether) to give 6-chloro-5-nitro-1-(tetrahydro-2H-pyran-2-yl)-1H-indazole (Int-15C). MS (ESI) [M+H]⁺: m/z 282. ¹H NMR (400 MHz, CDCl₃) δ 8.38 (s, 1H), 8.16 (s, 1H), 7.81 (s, 1H), 5.73 (dd, J=8.9, 2.5 Hz, 1H), 3.99-4.04 (m, 1H), 3.76-3.81 (m, 1H), 2.46-2.50 (m, 1H), 2.10-2.18 (m, 2H), 1.71-1.77 (m, 3H).

### Step D: 6-chloro-1-(tetrahydro-2H-pyran-2-yl)-1H-indazol-5-amine (Int-15D)

To a solution of 6-chloro-5-nitro-1-(tetrahydro-2H-pyran-2-yl)-1H-indazole (Int-15C) (38.6 g, 137 mmol) in EtOH (1.00 L) and water (200 mL) were added ammonium chloride (22.0 g, 411 mmol) and iron dust (38.3 g, 685 mmol) while stirring at 25° C. under N₂ atmosphere. The reaction mixture was stirred at 70° C. for 15 h. The reaction mixture was cooled, diluted with EtOAc (200 mL), filtered, and the solvent was concentrated in vacuo. The residue was then dissolved in EtOAc (3×300 mL), washed with brine (100 mL), dried over Na₂SO₄, filtered, and the solvent was evaporated under reduced pressure to give a crude product. The crude product was purified by flash silica gel chromatography (15% EtOAc in petroleum ether) to give 6-chloro-1-(tetrahydro-2H-pyran-2-yl)-1H-indazol-5-amine (Int-15D). MS (ESI) [M+H]⁺: m/z 252. ¹H NMR (400 MHz, CDCl₃) δ 7.82 (d, J=0.8 Hz, 1H), 7.60 (s, 1H), 7.04 (s, 1H), 5.61 (dd, J=9.4, 2.7 Hz, 1H), 4.02-4.07 (m, 1H), 3.89-4.01 (m, 2H), 3.71-3.77 (m, 1H), 2.49-2.56 (m, 1H), 2.06-2.17 (m, 2H), 1.68-1.79 (m, 3H).

### Step E: 4-bromo-6-chloro-1-(tetrahydro-2H-pyran-2-yl)-1H-indazol-5-amine (Int-15E)

To a solution of 6-chloro-1-(tetrahydro-2H-pyran-2-yl)-1H-indazol-5-amine (Int-15D) (66.6 g, 265 mmol) in MeCN (660 mL) was added NBS (56.5 g, 318 mmol) at 20° C. under N₂ atmosphere. The reaction mixture was stirred at 20° C. for 3 h. The reaction mixture was quenched with water (200 mL), diluted with EtOAc (200 mL), filtered, and concentrated. The residue was extracted with EtOAc (2×200 mL), washed with brine (100 mL), dried over Na₂SO₄, filtered and the solvent was evaporated under reduced pressure to give the crude product. The crude product was purified by flash silica gel chromatography (10% EtOAc in petroleum ether) to give 4-bromo-6-chloro-1-(tetrahydro-2H-pyran-2-yl)-1H-indazol-5-amine (Int-15E). MS (ESI) [M+H]⁺: m/z 330, 332. ¹H NMR (400 MHz, CDCl₃) δ 7.84 (s, 1H), 7.59 (s, 1H), 5.61 (dd, J=9.0, 2.7 Hz, 1H), 3.97-4.01 (m, 1H), 3.70-3.76 (m, 1H), 2.44-2.52 (m, 1H), 2.05-2.16 (m, 2H), 1.66-1.77 (m, 3H).

### Step F: 4-bromo-6-chloro-1H-indazol-5-amine (Int-15F)

To a solution of 4-bromo-6-chloro-1-(tetrahydro-2H-pyran-2-yl)-1H-indazol-5-amine (Int-7E) (30 g, 90 mmol) was added 4N HCl in MeOH (300 mL). The reaction was stirred at 50° C. for 2 h. The reaction mixture was cooled and evaporated under reduced pressure to give 4-bromo-6-chloro-1H-indazol-5-amine (Int-15F) isolated as an HCl salt. MS (ESI) [M+H]⁺: m/z 246, 248.

### Step G: 4-bromo-6-chloro-5-iodo-1H-indazole (Int-15G)

To a solution of 4-bromo-6-chloro-1H-indazol-5-amine (Int-15F), HCl (10 g, 35 mmol) in 6M aq. HCl (100 mL) was added a solution of sodium nitrite (2.9 g, 42 mmol) in water (20 mL) dropwise at −5° C. and stirred for 5 min. Then a solution of KI (23 g, 140 mmol) in water (100 mL) was added dropwise to the reaction mixture at −5° C. The reaction mixture was stirred at 90° C. for 1 h. The reaction was cooled, quenched with ice water (150 mL) and sat. aq. Na₂SO₃ (100 mL), and then basified with sat. aq. NaHCO₃ (200 mL) to pH 8. The aqueous layer was extracted with EtOAc (2×200 mL), and the organic layer was dried over Na₂SO₄, filtered, and the solvent was evaporated under reduced pressure to give 4-bromo-6-chloro-5-iodo-1H-indazole (Int-15G). MS (ESI) [M+H]⁺: m/z 357, 359. ¹H NMR (400 MHz, DMSO-d₆) δ 13.62 (br s, 1H), 8.00 (s, 1H), 7.88 (d, J=0.8 Hz, 1H).

### Step H: 4-bromo-6-chloro-5-iodo-1-(tetrahydro-2H-pyran-2-yl)-1H-indazole (Int-15H)

To a solution of 4-bromo-6-chloro-5-iodo-1H-indazole (Int-15G) (32 g, 90 mmol) in THF (300 mL) were added 4-methylbenzenesulfonic acid (1.5 g, 9.0 mmol) and 3,4-dihydro-2H-pyran (16 mL, 180 mmol) at 20° C. under N₂ atmosphere. The reaction mixture was stirred at 70° C. for 3 h. The reaction mixture was cooled, evaporated under reduced pressure to give a crude product. The crude product was purified by flash silica gel chromatography (10% THF in petroleum ether) to give 4-bromo-6-chloro-5-iodo-1-(tetrahydro-2H-pyran-2-yl)-1H-indazole (Int-15H). MS (ESI) [M+H]⁺: m/z 441, 443. ¹H NMR (400 MHz, CDCl₃) δ 7.93 (s, 1H), 7.79 (d, J=0.7 Hz, 1H), 5.65 (dd, J=8.8, 2.7 Hz, 1H), 3.96-4.00 (m, 1H), 3.71-3.77 (m, 1H), 2.42-2.50 (m, 1H), 2.07-2.16 (m, 2H), 1.69-1.79 (m, 3H).

### Step I: 4-bromo-6-chloro-1-(tetrahydro-2H-pyran-2-yl)-1H-indazole-5-carbaldehyde (Int-15I)

To a solution of 4-bromo-6-chloro-5-iodo-1-(tetrahydro-2H-pyran-2-yl)-1H-indazole (Int-7H) (8.0 g, 18.12 mmol) in THF (60 mL) and DMF (30 mL, 390 mmol) was added isopropylmagnesium chloride lithium chloride complex (15.3 mL, 20.0 mmol, 1.3 M in THF) at −5° C. The mixture was stirred at −5° C. for 10 min. Three batches were prepared in this fashion. The combined reactions were quenched with ice water (100 mL) and extracted with EtOAc (3×100 mL). The organic layer was washed with brine (50 mL), dried over sodium sulfate, filtered and concentrated in vacuo. The residue was purified by flash silica gel chromatography (0-15% THF in petroleum ether), followed by recrystallization with EtOAc/Pet. ether (˜10:1) to give 4-bromo-6-chloro-1-(tetrahydro-2H-pyran-2-yl)-1H-indazole-5-carbaldehyde (Int-15I). MS (ESI) [M+H]⁺: m/z 343, 345. ¹H NMR (400 MHz, CDCl₃) δ 10.51 (s, 1H), 8.19 (s, 1H), 7.71 (s, 1H), 5.70 (dd, J=8.9, 2.6 Hz, 1H), 3.99-4.03 (m, 1H), 3.74-3.80 (m, 1H), 2.42-2.49 (m, 1H), 2.08-2.16 (m, 2H), 1.70-1.79 (m, 3H).

### Step J: 4-bromo-6-chloro-5-(difluoromethyl)-1-(tetrahydro-2H-pyran-2-yl)-1H-indazole (Int-15J)

To a solution of 4-bromo-6-chloro-1-(tetrahydro-2H-pyran-2-yl)-1H-indazole-5-carbaldehyde (Int-15I) (8.0 g, 23 mmol) in DCM (100 mL) was added DAST (25 mL, 190 mmol) under N₂ atmosphere. The reaction was stirred at 20° C. for 16 h. The reaction was diluted with EtOAc (200 mL), quenched with sat. aq. NaHCO₃ (150 mL), the organic layer was washed with brine (50 mL), dried, and concentrated in vacuo. The residue was purified by flash silica gel chromatography (20% THF in petroleum ether) to give 4-bromo-6-chloro-5-(difluoromethyl)-1-(tetrahydro-2H-pyran-2-yl)-1H-indazole (Int-15J). MS (ESI) [M+H]⁺: m/z 365, 367. ¹H NMR (400 MHz, MeOD) δ 8.17 (s, 1H), 7.94 (s, 1H), 7.24-7.57 (m, 1H), 5.84 (dd, J=2.62, 9.18 Hz, 1H), 3.95 (br s, 1H), 3.84 (br d, J=13.23 Hz, 1H), 2.45 (br d, J=11.44 Hz, 1H), 2.02-2.18 (m, 2H), 1.64-1.89 (m, 3H).

### Step K: (6-chloro-5-(difluoromethyl)-1-(tetrahydro-2H-pyran-2-yl)-1H-indazol-4-yl)boronic acid (Int-15)

To a solution of 4-bromo-6-chloro-5-(difluoromethyl)-1-(tetrahydro-2H-pyran-2-yl)-1H-indazole (Int-15J) (2.0 g, 5.5 mmol) in MeOH (40 mL) was added tetrahydroxydiboron (2.0 g, 22 mmol)), triethylamine (2.3 mL, 16 mmol) and CataCXium A Pd G2 (0.18 g, 0.27 mmol). The mixture was stirred at 40° C. for 1 h under N₂ atmosphere. The reaction mixture was concentrated under reduced pressure. The residue was acidified with aqueous HCl (0.5 M) to pH ˜6 at 0° C., and the mixture was extracted with EtOAc (50 mL). The organic layer was washed with brine (3×30 mL), dried over Na₂SO₄, filtered and concentrated in vacuum. The residue was purified by flash silica gel chromatography (0-50% EtOH/EtOAc) to give (6-chloro-5-(difluoromethyl)-1-(tetrahydro-2H-pyran-2-yl)-1H-indazol-4-yl)boronic acid (Int-15). MS (ESD) [M+H]⁺: m/z 331. ¹H NMR (400 MHz, CDCl₃) δ 8.07-8.17 (m, 1H), 7.70-7.76 (m, 1H), 7.26-7.29 (m, 1H), 5.66-5.76 (m, 1H), 3.70-3.85 (m, 2H), 2.07 (d. J=4.53 Hz, 2H), 1.66-1.86 (m, 4H).

### Intermediate 16: (6-chloro-1-(tetrahydro-2H-pyran-2-yl)-5-((trimethylsilyl) ethynyl)-1H-indazol-4-yl)boronic acid (Int-16)

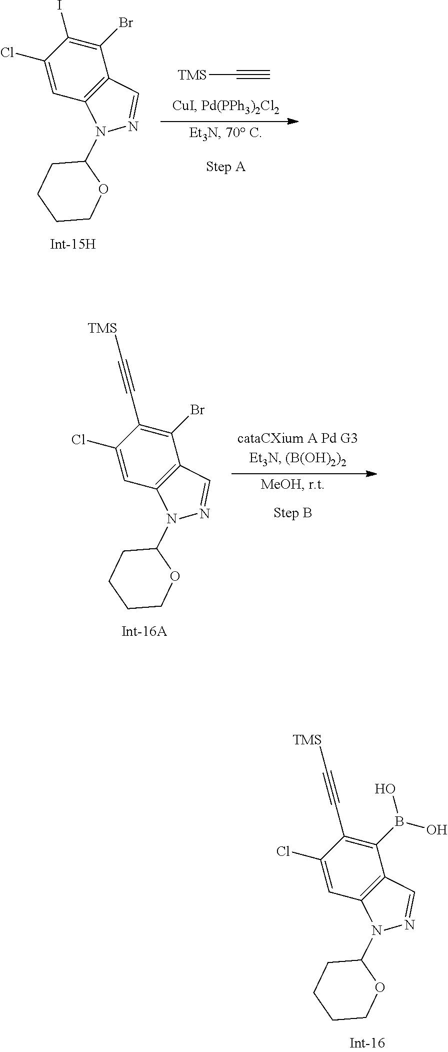

### Step A: 4-bromo-6-chloro-1-(tetrahydro-2H-pyran-2-yl)-5-((trimethylsilyl) ethynyl)-1H-indazole (Int-16A)

Trimethylsilylacetylene (1.72 mL, 12.2 mmol), Pd(PPh₃)₂Cl₂ (47.7 mg, 0.0700 mmol), and CuI (26 mg, 0.14 mmol) were added to a solution of 4-bromo-6-chloro-5-iodo-1-(tetrahydro-2H-pyran-2-yl)-1H-indazole (Int-15H) (600 mg, 1.36 mmol) in Et₃N (7.6 mL) at room temperature. The reaction was warmed to 70° C. and stirred for 10 h. The solution was diluted with EtOAc and filtered through CELITE®. The filtrate was washed with brine, dried over Na₂SO₄, filtered, and evaporated. The residue was purified by silica gel chromatography (hexane/EtOAc) to afford 4-bromo-6-chloro-1-(tetrahydro-2H-pyran-2-yl)-5-((trimethylsilyl) ethynyl)-1H-indazole (Int-16A). MS (ESI): m/z (M+H)⁺ 411, 413. ¹H NMR (400 MHz, CDCl₃) δ 7.98 (d, J=0.8 Hz, 1H), 7.65 (q, J=1.1 Hz, 1H), 5.63 (dd, J=9.0, 2.8 Hz, 1H), 4.01-3.97 (m, 1H), 3.76-3.73 (m, 1H), 2.48-2.41 (m, 1H), 2.17-2.05 (m, 2H), 1.78-1.67 (m, 3H), 0.33-0.30 (m, 9H).

### Step B: (6-chloro-1-(tetrahydro-2H-pyran-2-yl)-5-((trimethylsilyl) ethynyl)-1H-indazol-4-yl)boronic acid (Int-16)

Tetrahydroborate (51 mg, 0.57 mmol), cataCXium A Pd G3 (12 mg, 0.016 mmol) and Et₃N (130 μL, 0.91 mmol) were added to a solution of 4-bromo-6-chloro-1-(tetrahydro-2H-pyran-2-yl)-5-((trimethylsilyl) ethynyl)-1H-indazole (Int-16A) (94 mg, 0.23 mmol) in MeOH (2.3 mL). After stirring at room temperature for 1 h, the solution was filtered through CELITE® and evaporated. The residue was purified by silica gel chromatography (CHCl₃/EtOH) to afford (6-chloro-1-(tetrahydro-2H-pyran-2-yl)-5-((trimethylsilyl) ethynyl)-1H-indazol-4-yl)boronic acid (Int-16). MS (ESI): m/z (M+H)⁺ 377.

### Intermediate 17: (6-fluoro-5-(1-fluorocyclopropyl)-1-(tetrahydro-2H-pyran-2-yl)-1H-indazol-4-yl)boronic acid (Int-17)

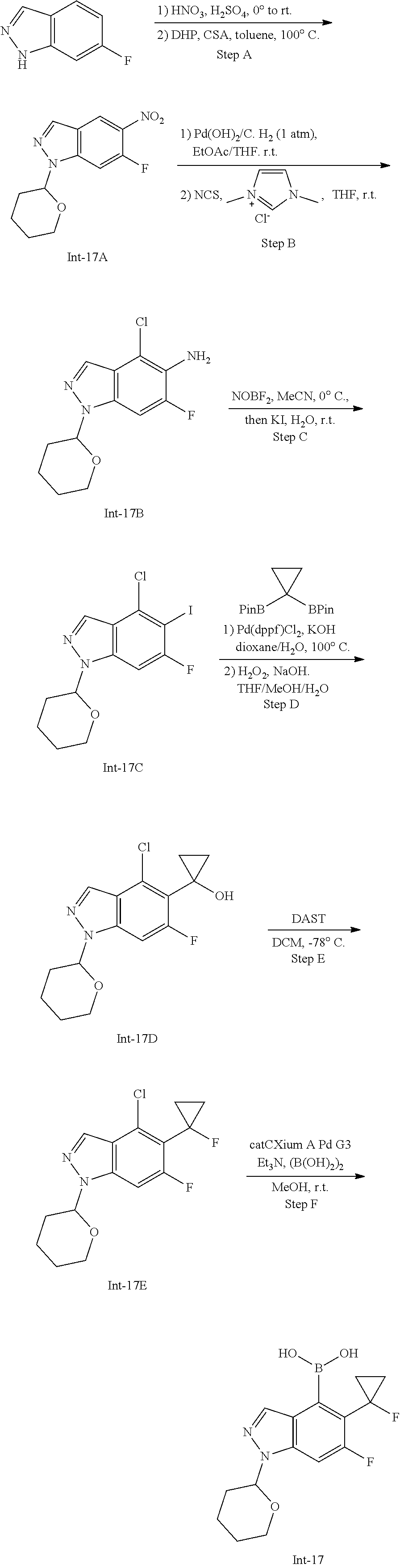

### Step A: 6-fluoro-5-nitro-1-(tetrahydro-2H-pyran-2-yl)-1H-indazole (Int-17A)

HNO₃ (1.0 mL, 22.2 mmol, 65 wt %) was slowly added to a solution of 6-fluoro-1H-indazole (2.72 g, 20.0 mmol) in H₂SO₄ (20 mL) at 0° C. The mixture was warmed to room temperature and stirred for 5 h. After 5 h, the mixture was slowly poured onto crushed ice, filtered, and the filter cake was washed with water. The filter cake was dissolved in EtOAc and THF, washed with brine, and dried over Na₂SO₄. The mixture was filtered and the filtrate was concentrated under reduced pressure to afford 6-fluoro-5-nitro-1H-indazole.

The product was dissolved in toluene (30 mL) and 3,4-dihydro-2H-pyran (6 mL, 66 mmol), and (1R)-(−)-camphor-10-sulfonic acid (464 mg, 2.00 mmol) were added. The reaction was heated to 100° C. for 6 b. After stirring for 6 h, the reaction mixture was cooled to room temperature and sat. aq. NaHCO₃ and EtOAc were added. The organics were separated, washed with brine, dried with Na₂SO₄, and concentrated. The residue was purified by silica gel chromatography (hexane-EtOAc) to give 6-fluoro-5-nitro-1-(tetrahydro-2H-pyran-2-yl)-1H-indazole (Int-17A). MS (ESI) [M+H]⁺: m/z 266.

### Step B: 4-chloro-6-fluoro-1-(tetrahydro-2H-pyran-2-yl)-1H-indazol-5-amine (Int-17B)

6-fluoro-5-nitro-1-(tetrahydro-2H-pyran-2-yl)-1H-indazole (Int-17A) (1.31 g, 4.94 mmol) was dissolved in THF (3 mL) and EtOAc (10 mL). The reaction was purged with nitrogen at 1 atm and palladium hydroxide on carbon (0.5 g) was added. The atmosphere was replaced with hydrogen and the reaction mixture was stirred at room temperature for 3 h. After 3 h, the reaction was filtered and concentrated in vacuo.

The crude residue was dissolved in THF (20 mL) and NCS (692 mg, 5.18 mmol) and 1,3-dimethylimidazolium chloride (66.0 mg, 0.498 mmol) were added to the solution. The reaction was stirred at room temperature overnight. Sat. aq. NaHCO₃ and EtOAc were added to the reaction, the organics were separated, washed with brine, dried with anhydrous Na₂SO₄, and concentrated. The crude residue was purified by silica gel chromatography (hexane-EtOAc) to give 4-chloro-6-fluoro-1-(tetrahydro-2H-pyran-2-yl)-1H-indazol-5-amine (Int-17B). MS (ESI) [M+H]⁺: m/z 270.

### Step C: 4-chloro-6-fluoro-5-iodo-1-(tetrahydro-2H-pyran-2-yl)-1H-indazole (Int-17C)

Nitrosyl tetrafluoroborate (411 mg, 3.52 mmol) was added to a solution of 4-chloro-6-fluoro-1-(tetrahydro-2H-pyran-2-yl)-1H-indazol-5-amine (Int-17B) (730 mg, 2.71 mmol) in MeCN (20 mL) at 0° C. and stirred for 5 min. Then, this reaction mixture was added to a solution of KI (10 g, 60.2 mmol) in water (30 mL) at room temperature and the reaction mixture was quenched with sat. aq. NaHCO₃ and Na₂S₂O₃. The layers were separated and the aqueous layer was extracted with EtOAc. The combined organic layers were dried over Na₂SO₄, filtered, and the solvent was evaporated under reduced pressure. The crude residue was purified by column chromatography (hexane-EtOAc) to afford 4-chloro-6-fluoro-5-iodo-1-(tetrahydro-2H-pyran-2-yl)-1H-indazole (Int-17C). MS (ESI) [M+H]⁺: m/z 381.

### Step D: 1-(4-chloro-6-fluoro-1-(tetrahydro-2H-pyran-2-yl)-1H-indazol-5-yl)cyclopropan-1-ol (Int-17D)

KOH (1.0 mL, 2.0 mmol, 2M in H₂O) was added to a mixture of 4-chloro-6-fluoro-5-iodo-1-(tetrahydro-2H-pyran-2-yl)-1H-indazole (Int-17C) (354 mg, 0.930 mmol), 4,4,5,5-tetramethyl-2-[1-(tetramethyl-1,3,2-dioxaborolan-2-yl)ethyl]-1,3,2-dioxaborolane (600 mg, 2.04 mmol), and [1,1′-bis(diphenylphosphino)ferrocene]dichloropalladium (II) (70 mg, 0.096 mmol) in 1,4-dioxane (8.0 mL). The mixture was heated to 100° C. and stirred for 18 h. After 18 h, the mixture was cooled to room temperature, quenched with sat. aq. NaHCO₃, and diluted with EtOAc. The organic layer was separated, washed with brine, and dried over Na₂SO₄. The mixture was filtered and the filtrate was concentrated under reduced pressure. The residue obtained was purified by column chromatography (hexane-EtOAc=95/5 to 50/50) to afford 4-chloro-6-fluoro-1-(tetrahydro-2H-pyran-2-yl)-5-(1-(4,4,5,5-tetramethyl-1,3,2-dioxaborolan-2-yl)cyclopropyl)-1H-indazole.

MeOH (10 mL) and NaOH (1.3 ml, 2.6 mmol, 2M in H₂O) were added to 4-chloro-6-fluoro-1-(tetrahydro-2H-pyran-2-yl)-5-(1-(4,4,5,5-tetramethyl-1,3,2-dioxaborolan-2-yl)cyclopropyl)-1H-indazole at 0° C. H₂O₂ (0.3 mL, 30 wt %) was added slowly to this mixture at 0° C. The reaction was stirred at 0° C. for 30 min, then water and chloroform were added. The organics were separated, washed with brine, dried with anhydrous Na₂SO₄, and concentrated. The residue was purified by silica gel chromatography (hexane-EtOAc) to afford 1-(4-chloro-6-fluoro-1-(tetrahydro-2H-pyran-2-yl)-1H-indazol-5-yl)cyclopropan-1-ol (Int-17D). MS (ESI) [M+H]⁺: m/z 355.

### Step E: 4-chloro-6-fluoro-5-(1-fluorocyclopropyl)-1-(tetrahydro-2H-pyran-2-yl)-1H-indazole (Int-17E)

Diethylaminosulfur trifluoride (0.120 mL, 0.908 mmol) was added to a solution of 1-(4-chloro-6-fluoro-1-(tetrahydro-2H-pyran-2-yl)-1H-indazol-5-yl)cyclopropan-1-ol (Int-17D) (132 mg, 0.425 mmol) in DCM (5 mL) at −78° C. After stirring at −78° C. for 15 min, the reaction mixture was diluted with sat. aq. NaHCO₃ and EtOAc. The organics were separated, washed with brine, dried with anhydrous Na₂SO₄, and concentrated. The residue was purified by silica gel chromatography (hexane-EtOAc) to give 4-chloro-6-fluoro-5-(1-fluorocyclopropyl)-1-(tetrahydro-2H-pyran-2-yl)-1H-indazole (Int-17E). MS (ESI) [M+H]⁺: m/z 357.

### Step F: (6-fluoro-5-(1-fluorocyclopropyl)-1-(tetrahydro-2H-pyran-2-yl)-1H-indazol-4-yl)boronic acid (Int-17)

Tetrahydroxydiboron (120 mg, 1.34 mmol), triethylamine (0.26 mL, 1.9 mmol), and cataCXium A Pd G3 (0.017 g, 0.023 mmol) were added to a solution of 4-chloro-6-fluoro-5-(1-fluorocyclopropyl)-1-(tetrahydro-2H-pyran-2-yl)-1H-indazole (Int-17E) (89 mg, 0.29 mmol) in MeOH (3 mL) at room temperature. After stirring at room temperature for 1.5 h under N₂ atmosphere, the reaction was diluted with H₃PO₄ (10% aq.) and CHCl₃. The organics were separated, washed with brine, dried with anhydrous Na₂SO₄, and concentrated. The residue was purified by flash silica gel chromatography (hexane-EtOAc/ethanol (4/1)) to afford (6-fluoro-5-(1-fluorocyclopropyl)-1-(tetrahydro-2H-pyran-2-yl)-1H-indazol-4-yl)boronic acid (Int-17). MS (ESI) [M+H]⁺: m/z 323.

### Intermediate 18: (6-chloro-5-isopropyl-1-(tetrahydro-2H-pyran-2-yl)-1H-indazol-4-yl)boronic acid (Int-18)

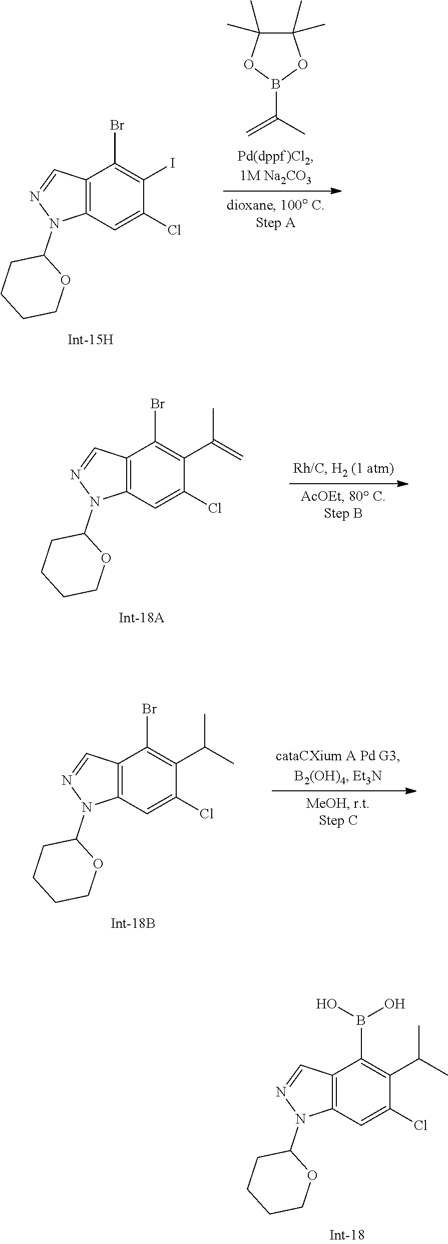

### Step A: 4-bromo-6-chloro-5-(prop-1-en-2-yl)-1-(tetrahydro-2H-pyran-2-yl)-1H-indazole (Int-18A)

A mixture of 4-bromo-6-chloro-5-iodo-1-(tetrahydro-2H-pyran-2-yl)-1H-indazole (Int-15H) (300 mg, 0.680 mmol), 4,4,5,5-tetramethyl-2-(prop-1-en-2-yl)-1,3,2-dioxaborolane (0.141 ml, 0.747 mmol), [1,1′-bis(diphenylphosphino)ferrocene]dichloropalladium (II) (50 mg, 0.068 mmol), sodium carbonate (3.34 ml, 3.34 mmol, 1 M in H₂O), and dioxane (6.7 ml) was degassed and N₂ charged (3×). Then the mixture was stirred at 100° C. under N₂ atmosphere for 1.5 h. After cooling, the mixture was partitioned between EtOAc and water. The organic phase was washed with brine, dried over sodium sulfate, filtered, and concentrated. The crude residue was purified via silica gel chromatography (0 to 10% EtOAc in hexane) to give (4-bromo-6-chloro-5-(prop-1-en-2-yl)-1-(tetrahydro-2H-pyran-2-yl)-1H-indazole (Int-18A). MS (ESI): m/z [M+H]⁺ 355, 357. ¹H NMR (400 MHz, CDCl₃) δ=8.01 (s, 1H), 7.67 (s, 1H), 5.67 (dd, J=2.8, 9.1 Hz, 1H), 5.45 (t, J=1.6 Hz, 1H), 4.96 (s, 1H), 4.06-3.99 (m, 1H), 3.81-3.71 (m, 1H), 2.58-2.44 (m, 1H), 2.22-2.10 (m, 1H), 2.08 (s, 3H), 1.85-1.67 (m, 4H).

### Step B: 4-bromo-6-chloro-5-isopropyl-1-(tetrahydro-2H-pyran-2-yl)-1H-indazole (Int-18B)

A mixture of (4-bromo-6-chloro-5-(prop-1-en-2-yl)-1-(tetrahydro-2H-pyran-2-yl)-1H-indazole (Int-18A) (200 mg, 0.562 mmol), rhodium on carbon (500 mg, 5 wt %) in EtOAc (5.6 ml) was degassed and charged with H₂ (3×) at 1 atm. The mixture was heated to 80° C. and stirred for 5 h. After cooling, the insoluble materials were removed by filtration through a CELITE® pad and the filtrate was concentrated. The crude residue was purified via silica gel chromatography (0 to 15% EtOAc in hexane) to give 4-bromo-6-chloro-5-isopropyl-1-(tetrahydro-2H-pyran-2-yl)-1H-indazole (Int-18B). MS (ESI): m/z [M+H]⁺359. ¹H NMR (400 MHz, CDCl₃) δ=8.03-7.95 (m, 1H), 7.67-7.57 (m, 1H), 5.64 (dd, J=2.8, 9.3 Hz, 1H), 4.08-3.95 (m, 2H), 3.85-3.68 (m, 1H), 2.58-2.43 (m, 1H), 2.18-2.03 (m, 2H), 1.84-1.65 (m, 3H), 1.53-1.42 (m, 6H).

### Step C: (6-chloro-5-isopropyl-1-(tetrahydro-2H-pyran-2-yl)-1H-indazol-4-yl)boronic acid (Int-18)

A mixture of 4-bromo-6-chloro-5-isopropyl-1-(tetrahydro-2H-pyran-2-yl)-1H-indazole (Int-18B) (140 mg, 0.39 mmol), tetrahydroxydiboron (70 mg, 0.78 mmol), cataCXium A Pd G3 (28.3 mg, 0.038 mmol) and triethylamine (0.22 ml, 1.6 mmol) in methanol (3.9 ml) was degassed and charged with N₂ (3×). Then the mixture was stirred at r.t. under N₂ atmosphere for 2 h. The insoluble materials were removed by filtration through a CELITE® pad and the filtrate was concentrated. The crude residue was purified via silica gel chromatography (20 to 100% EtOAc in hexane) to give (6-chloro-5-isopropyl-1-(tetrahydro-2H-pyran-2-yl)-1H-indazol-4-yl)boronic acid (Int-18). MS (ESI): m/z [M+H]⁺323. ¹H NMR (400 MHz, CDCl₃) δ=7.88 (s, 1H), 7.67 (s, 1H), 5.65 (dd, J=2.7, 9.4 Hz, 1H), 4.95 (s, 2H), 4.07-4.00 (m, 1H), 3.81-3.71 (m, 1H), 3.67-3.55 (m, 1H), 2.57-2.45 (m, 1H), 2.20-2.11 (m, 1H), 2.10-2.01 (m, 1H), 1.85-1.63 (m, 3H), 1.45 (s, 3H), 1.44 (s, 3H).

The compounds in the table below were synthesized using a similar procedure as described in the synthesis of Int-18 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-19 | 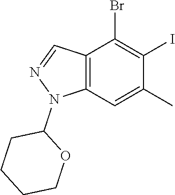 [SMILES: Cc1cc2c(cnn2C2CCCCO2)c(Br)c1I] Int-4C | 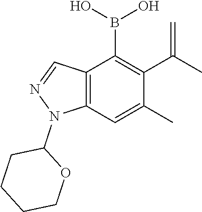 [SMILES: C=C(C)c1c(C)cc2c(cnn2C2CCCCO2)c1B(O)O] | (6-methyl-5-(prop- 1-en-2-yl)- 1-(tetrahydro-2H- pyran-2-yl)- 1H-indazol-4- yl)boronic acid | 301 |
| Int-20 | 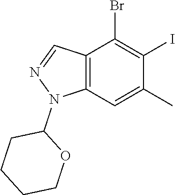 [SMILES: Cc1cc2c(cnn2C2CCCCO2)c(Br)c1I] Int-4C | 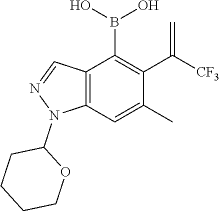 [SMILES: C=C(c1c(C)cc2c(cnn2C2CCCCO2)c1B(O)O)C(F)(F)F] | (6-methyl-1- (tetrahydro-2H- pyran-2-yl)-5- (3,3,3-trifluoro- prop-1-en-2-yl)- 1H-indazol-4- yl)boronic acid | 355 |
| Int-21 | 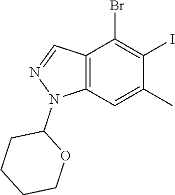 [SMILES: Cc1cc2c(cnn2C2CCCCO2)c(Br)c1I] Int-4C | 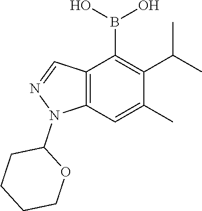 [SMILES: Cc1cc2c(cnn2C2CCCCO2)c(B(O)O)c1C(C)C] | (5-isopropyl-6- methyl-1- (tetrahydro-2H- pyran-2-yl)- 1H-indazol-4- yl)boronic acid | 303 |
| Int-22 | 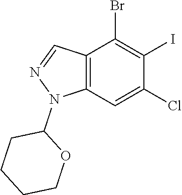 [SMILES: Clc1cc2c(cnn2C2CCCCO2)c(Br)c1I] Int-15H | 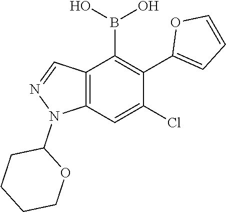 [SMILES: OB(O)c1c(-c2ccco2)c(Cl)cc2c1cnn2C1CCCCO1] | (6-chloro-5- (furan-2-yl)-1- (tetrahydro-2H- pyran-2-yl)- 1H-indazol-4- yl)boronic acid | 347 |
| Int-23 | 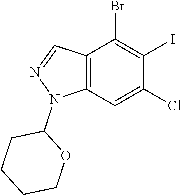 [SMILES: Clc1cc2c(cnn2C2CCCCO2)c(Br)c1I] Int-15H | 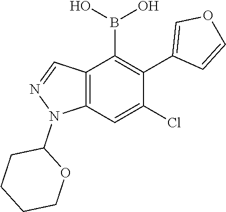 [SMILES: OB(O)c1c(-c2ccoc2)c(Cl)cc2c1cnn2C1CCCCO1] | (6-chloro-5- (furan-3-yl)-1- (tetrahydro-2H- pyran-2-yl)- 1H-indazol-4- yl)boronic acid | 347 |
| Int-24 | 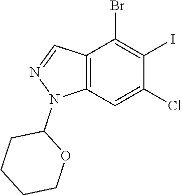 [SMILES: Clc1cc2c(cnn2C2CCCCO2)c(Br)c1I] Int-15H | 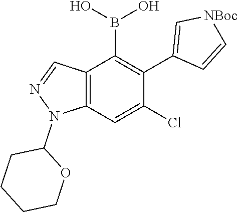 [SMILES: CC(C)(C)OC(=O)n1ccc(-c2c(Cl)cc3c(cnn3C3CCCCO3)c2B(O)O)c1] | (5-(1-(tert- butoxycarbonyl)- 1H-pyrrol-3-yl)- 6-chloro-1- (tetrahydro-2H- pyran-2-yl)- 1H-indazol-4- yl)boronic acid | 446 |
| Int-25 | 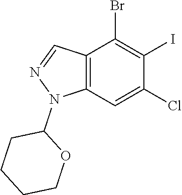 [SMILES: Clc1cc2c(cnn2C2CCCCO2)c(Br)c1I] Int-15H | 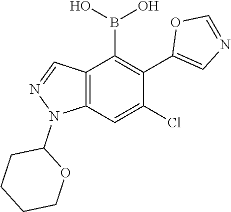 [SMILES: OB(O)c1c(-c2cnco2)c(Cl)cc2c1cnn2C1CCCCO1] | (6-chloro-5- (oxazol-5-yl)-1- (tetrahydro-2H- pyran-2-yl)- 1H-indazol-4- yl)boronic acid | 348 |
| Int-26 | 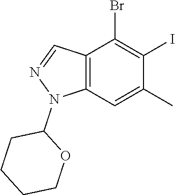 [SMILES: Cc1cc2c(cnn2C2CCCCO2)c(Br)c1I] Int-4C | 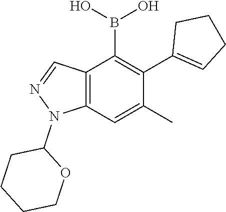 [SMILES: Cc1cc2c(cnn2C2CCCCO2)c(B(O)O)c1C1=CCCC1] | (5-(cyclopent-1- en-1-yl)-6- methyl-1- (tetrahydro-2H- pyran-2-yl)- 1H-indazol-4- yl)boronic acid | 327 |
| Int-27 | 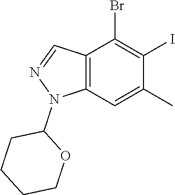 [SMILES: Cc1cc2c(cnn2C2CCCCO2)c(Br)c1I] Int-4C | 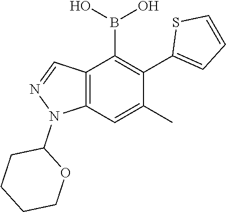 [SMILES: Cc1cc2c(cnn2C2CCCCO2)c(B(O)O)c1-c1cccs1] | (6-methyl-1- (tetrahydro-2H- pyran-2-yl)-5- (thiophen-2-yl)- 1H-indazol-4- yl)boronic acid | 343 |
| Int-28 | 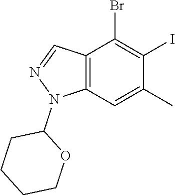 [SMILES: Cc1cc2c(cnn2C2CCCCO2)c(Br)c1I] Int-4C | 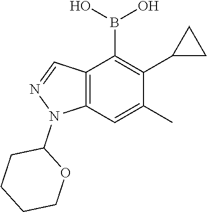 [SMILES: Cc1cc2c(cnn2C2CCCCO2)c(B(O)O)c1C1CC1] | (5-cyclopropyl- 6-methyl-1- (tetrahydro-2H- pyran-2-yl)-1H- indazol-4-yl) boronic acid | 301 |
| Int-29 | 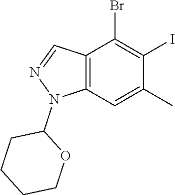 [SMILES: Cc1cc2c(cnn2C2CCCCO2)c(Br)c1I] Int-4C | 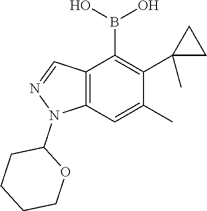 [SMILES: Cc1cc2c(cnn2C2CCCCO2)c(B(O)O)c1C1(C)CC1] | (6-methyl-5-(1- methylcyclopropyl)- 1-(tetrahydro-2H- pyran-2-yl)- 1H-indazol-4- yl)boronic acid | 315 |
| Int-30 | 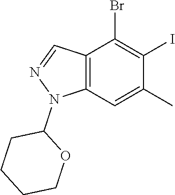 [SMILES: Cc1cc2c(cnn2C2CCCCO2)c(Br)c1I] Int-4C | 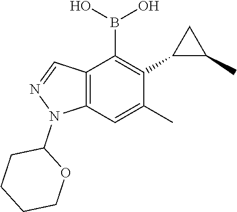 [SMILES: Cc1cc2c(cnn2C2CCCCO2)c(B(O)O)c1[C@@H]1C[C@H]1C] | (6-methyl-5- ((1R,2R)-2- methylcyclopropyl)- 1-(tetrahydro-2H- pyran-2-yl)-1H- 1H-indazol-4- yl)boronic acid | 315 |
| Int-31 | 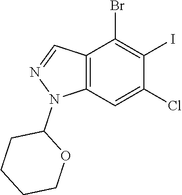 [SMILES: Clc1cc2c(cnn2C2CCCCO2)c(Br)c1I] Int-15H | 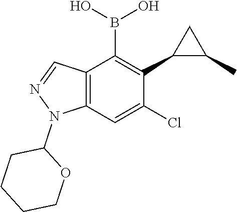 [SMILES: C[C@@H]1C[C@@H]1c1c(Cl)cc2c(cnn2C2CCCCO2)c1B(O)O] | (6-chloro-5- ((1S,2R)-2- methylcyclopropyl)- 1-(tetrahydro-2H- pyran-2-yl)- 1H-indazol-4- yl)boronic acid | 335 |
| Int-32 | 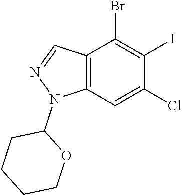 [SMILES: Clc1cc2c(cnn2C2CCCCO2)c(Br)c1I] Int-15H | 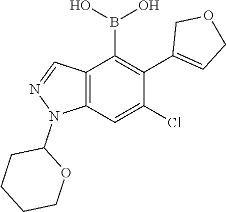 [SMILES: OB(O)c1c(C2=CCOC2)c(Cl)cc2c1cnn2C1CCCCO1] | (6-chloro-5-(2,5- dihydrofuran-3- yl)-1-(tetrahydro- 2H-pyran-2-yl)- 1H-indazol-4- yl)boronic acid | 349 |
| Int-33 | 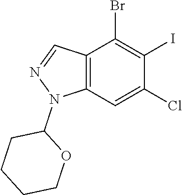 [SMILES: Clc1cc2c(cnn2C2CCCCO2)c(Br)c1I] Int-15H | 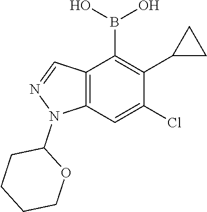 [SMILES: OB(O)c1c(C2CC2)c(Cl)cc2c1cnn2C1CCCCO1] | (6-chloro-5- cyclopropyl-1- (tetrahydro-2H- pyran-2-yl)- 1H-indazol-4- yl)boronic acid | 321 |
| Int-34 | 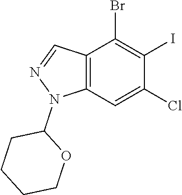 [SMILES: Clc1cc2c(cnn2C2CCCCO2)c(Br)c1I] Int-15H | 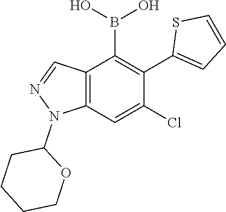 [SMILES: OB(O)c1c(-c2cccs2)c(Cl)cc2c1cnn2C1CCCCO1] | (6-chloro-1- (tetrahydro-2H- pyran-2-yl)-5- (thiophen-2-yl)- 1H-indazol-4- yl)boronic acid | 363 |
| Int-35 | 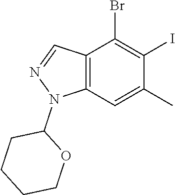 [SMILES: Cc1cc2c(cnn2C2CCCCO2)c(Br)c1I] Int-4C | 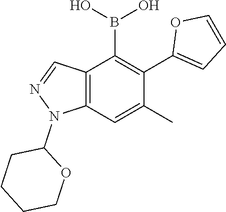 [SMILES: Cc1cc2c(cnn2C2CCCCO2)c(B(O)O)c1-c1ccco1] | (5-(furan-2-yl)-6- methyl-1- (tetrahydro-2H- pyran-2-yl)- 1H-indazol-4- yl)boronic acid | 327 |
| Int-36 | 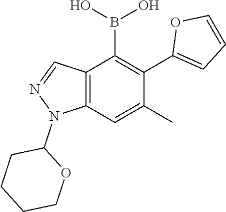 [SMILES: Cc1cc2c(cnn2C2CCCCO2)c(B(O)O)c1-c1ccco1] Int-4C | 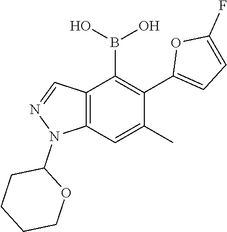 [SMILES: Cc1cc2c(cnn2C2CCCCO2)c(B(O)O)c1-c1ccc(F)o1] | (5-(5-fluorofuran-2- yl)-6-methyl-1- (tetrahydro-2H- pyran-2-yl)- 1H-indazol-4- yl)boronic acid | 345 |
| Int-37 | 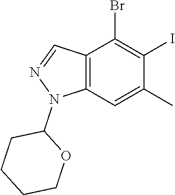 [SMILES: Cc1cc2c(cnn2C2CCCCO2)c(Br)c1I] Int-4C | 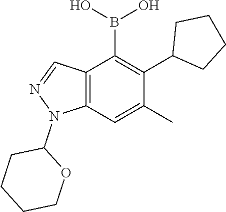 [SMILES: Cc1cc2c(cnn2C2CCCCO2)c(B(O)O)c1C1CCCC1] | (5-cyclopentyl-6- methyl-1- (tetrahydro-2H- pyran-2-yl)- 1H-indazol-4- yl)boronic acid | 329 |
| Int-38 | 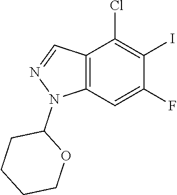 [SMILES: Fc1cc2c(cnn2C2CCCCO2)c(Cl)c1I] Int-17C | 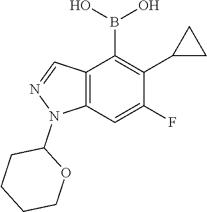 [SMILES: OB(O)c1c(C2CC2)c(F)cc2c1cnn2C1CCCCO1] | (5-cyclopropyl-6- fluoro-1- (tetrahydro-2H- pyran-2-yl)- 1H-indazol-4- yl)boronic acid | 305 |

### Intermediate 39: 4,4-difluoro-5-methyl-7-(tetrahydro-2H-pyran-2-yl)-4,7-dihydro-[1,2]oxaborinino[3,4-e]indazol-1(3H)-ol (Int-39)

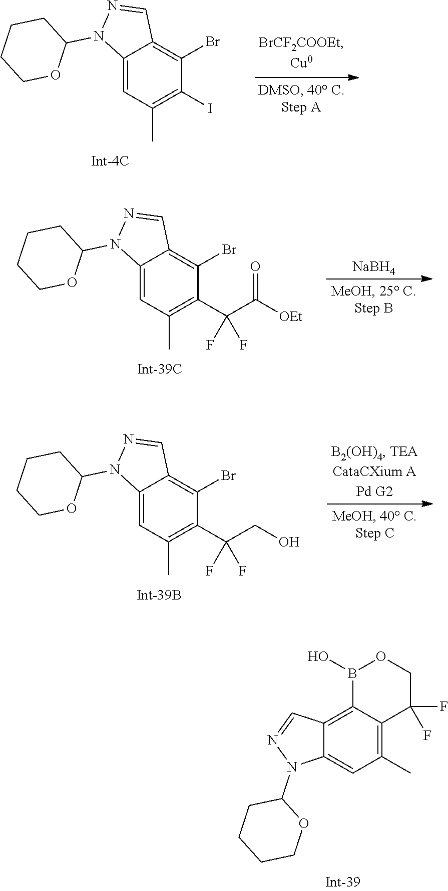 [SMILES: Cc1cc2c(cnn2C2CCCCO2)c(Br)c1I ; CCOC(=O)C(F)(F)c1c(C)cc2c(cnn2C2CCCCO2)c1Br ; "NaBH4" ; Cc1cc2c(cnn2C2CCCCO2)c(Br)c1C(F)(F)CO ; "B2(OH)4, TEACataCXium APd G2" ; Cc1cc2c(cnn2C2CCCCO2)c2c1C(F)(F)COB2O]

### Step A: Ethyl 2-(4-bromo-6-methyl-1-(tetrahydro-2H-pyran-2-yl)-1H-indazol-5-yl)-2,2-difluoroacetate (Int-39A)

To a solution of 4-bromo-5-iodo-6-methyl-1-(tetrahydro-2H-pyran-2-yl)-1H-indazole (Int-4C) (2 g, 4.75 mmol) in DMSO (15 mL) was added BrCF₂COOEt (2.89 g, 14.25 mmol) and copper powder (1.207 g, 19.00 mmol) at 25° C. under nitrogen atmosphere. The mixture was stirred at 40° C. for 16 h. The mixture was cooled, diluted with water (10 mL), extracted with EtOAc (3×10 mL), dried over sodium sulfate, filtered, and the solvent was evaporated under reduced pressure to give the crude product. The crude product was purified by flash silica gel chromatography (10% EtOAc in petroleum ether) to give ethyl 2-(4-bromo-6-methyl-1-(tetrahydro-2H-pyran-2-yl)-1H-indazol-5-yl)-2,2-difluoroacetate (Int-39A). MS (ESI) [M+H]⁺: m/z 417, 419.

### Step B: 2-(4-bromo-6-methyl-1-(tetrahydro-2H-pyran-2-yl)-1H-indazol-5-yl)-2,2-difluoroethan-1-ol (Int-39B)

To a solution of ethyl 2-(4-bromo-6-methyl-1-(tetrahydro-2H-pyran-2-yl)-1H-indazol-5-yl)-2,2-difluoroacetate (Int-39A) (1.548 g, 3.71 mmol) in MeOH (15 mL) was added sodium borohydride (0.561 g, 14.84 mmol) at 25° C. The mixture was stirred at 25° C. for 1 h. The mixture was quenched with water (20 mL), extracted with EtOAc (3×10 mL), dried over sodium sulfate, filtered, and the solvent was evaporated under reduced pressure to give the crude product. The crude product was purified by flash silica gel chromatography (0 to 20% EtOAc in petroleum ether) to give 2-(4-bromo-6-methyl-1-(tetrahydro-2H-pyran-2-yl)-1H-indazol-5-yl)-2,2-difluoroethan-1-ol (Int-39B). MS (ESI) [M+H]⁺: m/z 375, 377.

### Step C: 4,4-difluoro-5-methyl-7-(tetrahydro-2H-pyran-2-yl)-4,7-dihydro-[1,2]oxaborinino[3,4-e]indazol-1 (3H)-ol (Int-39)

To a solution of 2-(4-bromo-6-methyl-1-(tetrahydro-2H-pyran-2-yl)-1H-indazol-5-yl)-2,2-difluoroethan-1-ol (Int-39B) (681 mg, 1.815 mmol), cataCXium A Pd G2 (60.7 mg, 0.091 mmol) in MeOH (7 mL) was added Et₃N (0.759 mL, 5.44 mmol) and hypodiboric acid (651 mg, 7.26 mmol) at 25° C. under nitrogen atmosphere. The mixture was stirred at 40° C. for 1 h. The mixture was evaporated under reduced pressure to give the crude product. The residue was purified by preparative reverse phase HPLC (MeCN/H₂O w/formic acid modifier) to give 4,4-difluoro-5-methyl-7-(tetrahydro-2H-pyran-2-yl)-4,7-dihydro-[1,2]oxaborinino[3,4-e]indazol-1 (3H)-ol (Int-39). MS (ESI) [M+H]⁺: m/z 323.

### Intermediate 40: (5-(1,1-difluoroethyl)-6-methyl-1-(tetrahydro-2H-pyran-2-yl)-1H-pyrazolo[3,4-b]pyridin-4-yl)boronic acid (Int-40)

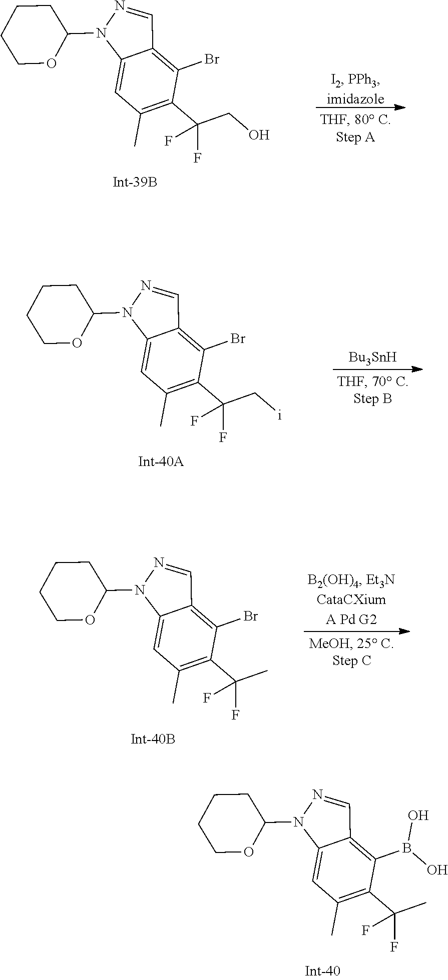

### Step A: 4-bromo-5-(1,1-difluoro-2-iodoethyl)-6-methyl-1-(tetrahydro-2H-pyran-2-yl)-1H-indazole (Int-40A)

To a solution of 2-(4-bromo-6-methyl-1-(tetrahydro-2H-pyran-2-yl)-1H-indazol-5-yl)-2,2-difluoroethan-1-ol (Int-39B) (400 mg, 1.066 mmol) in THF (6 mL) was added imidazole (290 mg, 4.26 mmol) and triphenylphosphine (1.12 g, 4.26 mmol) at 25° C. The reaction mixture was stirred at 25° C. for 20 min. I₂ (1082 mg, 4.26 mmol) was added at 25° C., and then the reaction was stirred at 80° C. for 12 h. The reaction was quenched with aq. Na₂SO₃ (3 mL) and extracted with EtOAc (3×5 mL). The organic layer was dried over sodium sulfate, filtered, and the solvent was evaporated under reduced pressure to give the crude product. The crude product was purified by preparative TLC (SiO₂, petroleum ether:EtOAc=5:1) to give 4-bromo-5-(1,1-difluoro-2-iodoethyl)-6-methyl-1-(tetrahydro-2H-pyran-2-yl)-1H-indazole (Int-40A). MS (ESI) [M+H]⁺: m/z 485, 487.

### Step B: 4-bromo-5-(1,1-difluoroethyl)-6-methyl-1-(tetrahydro-2H-pyran-2-yl)-1H-indazole (Int-40B)

To a solution of 4-bromo-5-(1,1-difluoro-2-iodoethyl)-6-methyl-1-(tetrahydro-2H-pyran-2-yl)-1H-indazole (Int-40A) (400 mg, 0.825 mmol) in THF (8 mL) was added tri-n-butyltin hydride (2 g, 6.87 mmol) at 25° C. under N₂. The reaction mixture was stirred at 70° C. for 2 h. The reaction solution was cooled, quenched with aq. KF (10 mL), and extracted with EtOAc (3×10 mL). The organic layer was dried over sodium sulfate, filtered, and the solvent was evaporated under reduced pressure to give the crude product. The crude product was purified by flash silica gel chromatography (0 to 7% EtOAc in petroleum ether) to give 4-bromo-5-(1,1-difluoroethyl)-6-methyl-1-(tetrahydro-2H-pyran-2-yl)-1H-indazole (Int-40B). MS (ESI) [M+H]⁺: m/z 359, 361.

### Step C: (5-(1,1-difluoroethyl)-6-methyl-1-(tetrahydro-2H-pyran-2-yl)-1H-pyrazolo[3,4-b]pyridin-4-yl)boronic acid (Int-40)

To a solution of 4-bromo-5-(1,1-difluoroethyl)-6-methyl-1-(tetrahydro-2H-pyran-2-yl)-1H-indazole (Int-40B) (75 mg, 0.208 mmol) in MeOH (1 mL) was added hypodiboric acid (74.7 mg, 0.833 mmol), cataCXium A Pd G2 (8.47 mg, 10.41 μmol), and triethylamine (0.087 mL, 0.625 mmol) at 25° C. under N₂. The reaction mixture was stirred at 25° C. for 1 h. The reaction mixture was diluted with DCM (5 mL), filtered, and the solvent was evaporated under reduced pressure to give the crude product. The crude product was purified by preparative TLC (SiO₂, petroleum ether:EtOAc=5:1) to give (5-(1,1-difluoroethyl)-6-methyl-1-(tetrahydro-2H-pyran-2-yl)-1H-pyrazolo[3,4-b]pyridin-4-yl)boronic acid (Int-40). MS (ESI) [M+H]⁺: m/z 325.

### Intermediate 41:2-methyl-2-(6-methyl-1-(tetrahydro-2H-pyran-2-yl)-4-(4,4,5,5-tetramethyl-1,3,2-dioxaborolan-2-yl)-1H-indazol-5-yl) propanenitrile (Int-41)

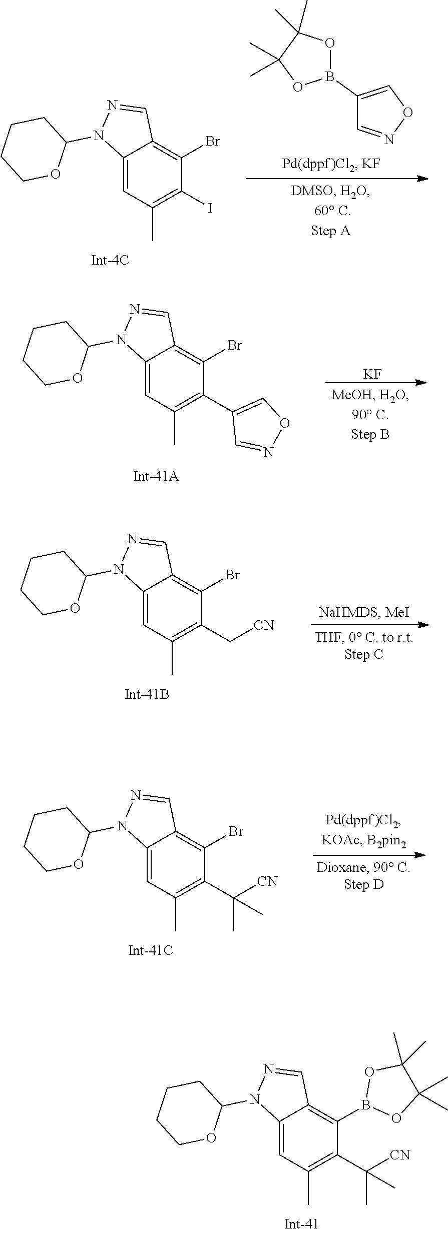

### Step A: 4-(4-bromo-6-methyl-1-(tetrahydro-2H-pyran-2-yl)-1H-indazol-5-ylisoxazole (Int-41A)

To a solution of 4-(4,4,5,5-tetramethyl-1,3,2-dioxaborolan-2-yl) isoxazole (2.038 g, 10.45 mmol), 4-bromo-5-iodo-6-methyl-1-(tetrahydro-2H-pyran-2-yl)-1H-indazole (Int-4C) (4 g, 9.50 mmol) in DMSO (10 mL) and water (2 mL) was added KF (1.656 g, 28.5 mmol) and Pd(dppf)Cl₂ (0.695 g, 0.950 mmol) at 25° C. under N₂. The mixture was stirred at 60° C. for 11 h. The mixture was cooled, diluted with water (10 mL), and extracted with EtOAc (3×10 mL). The organic layer was dried over sodium sulfate, filtered, and the solvent was evaporated under reduced pressure. The crude product was purified by flash silica gel chromatography (0 to 30% EtOAc in petroleum ether) to give 4-(4-bromo-6-methyl-1-(tetrahydro-2H-pyran-2-yl)-1H-indazol-5-yl) isoxazole (Int-41A). MS (ESI) [M+H]⁺: m/z 362, 364.

### Step B: 2-(4-bromo-6-methyl-1-(tetrahydro-2H-pyran-2-yl)-1H-indazol-5-yl) acetonitrile (Int-41B)

To a solution of 4-(4-bromo-6-methyl-1-(tetrahydro-2H-pyran-2-yl)-1H-indazol-5-yl) isoxazole (Int-41A) (1.7 g, 4.69 mmol) in MeOH (20 mL) and water (10 mL) was added KF (1.363 g, 23.47 mmol) at 25° C. The mixture was stirred at 90° C. for 12 b. The mixture was cooled, diluted with water (20 mL), and extracted with EtOAc (3×20 mL). The organic layer was washed with brine (2×50 mL), dried over sodium sulfate, filtered, and the solvent was evaporated under reduced pressure to give the crude product. The crude product was purified by flash silica gel chromatography (0 to 30% EtOAc in petroleum ether) to give 2-(4-bromo-6-methyl-1-(tetrahydro-2H-pyran-2-yl)-1H-indazol-5-yl) acetonitrile (Int-41B). MS (ESI) [M+H]⁺: m/z 334, 336.

### Step C: 2-(4-bromo-6-methyl-1-(tetrahydro-2H-pyran-2-yl)-1H-indazol-5-yl)-2-methylpropanenitrile (Int-41C)

To a solution of 2-(4-bromo-6-methyl-1-(tetrahydro-2H-pyran-2-yl)-1H-indazol-5-yl) acetonitrile (Int-41B) (535 mg, 1.601 mmol) in THF (5 mL) was added NaHMDS (4.80 mL, 4.80 mmol) at 0° C. under N₂. The mixture was stirred at 0° C. for 20 min, and then MeI (0.801 mL, 12.81 mmol) was added. The resulting mixture was stirred at 25° C. for 1 h. The mixture was quenched with sat. aq. NH₄Cl (5 mL), extracted with EtOAc (3×5 mL), dried over sodium sulfate, filtered, and the solvent was evaporated under reduced pressure. The crude product was purified by flash silica gel chromatography (0 to 30% EtOAc in petroleum ether) to give 2-(4-bromo-6-methyl-1-(tetrahydro-2H-pyran-2-yl)-1H-indazol-5-yl)-2-methylpropanenitrile (Int-41C). MS (ESI) [M+H]⁺: m/z 362, 364.

### Step D: 2-methyl-2-(6-methyl-1-(tetrahydro-2H-pyran-2-yl)-4-(4,4,5,5-tetramethyl-1,3,2-dioxaborolan-2-yl)-1H-indazol-5-yl) propanenitrile (Int-41)

To a solution of 2-(4-bromo-6-methyl-1-(tetrahydro-2H-pyran-2-yl)-1H-indazol-5-yl)-2-methylpropanenitrile (Int-41C) (541 mg, 1.493 mmol) and bis(pinacolato)diboron (758 mg, 2.99 mmol) in dioxane (6 mL) was added KOAc (440 mg, 4.48 mmol) and Pd(dppf)Cl₂ (109 mg, 0.149 mmol) at 25° C. under N₂. The mixture was stirred at 90° C. for 1 h. The mixture was cooled, diluted with water (6 mL), and extracted with EtOAc (3×6 mL). The organic layer was dried over sodium sulfate, filtered, and the solvent was evaporated under reduced pressure. The residue was purified by preparative reverse-phase HPLC (MeCN/H₂O w/formic acid modifier) to give 2-methyl-2-(6-methyl-1-(tetrahydro-2H-pyran-2-yl)-4-(4,4,5,5-tetramethyl-1,3,2-dioxaborolan-2-yl)-1H-indazol-5-yl) propanenitrile (Int-41). MS (ESI) [M+H]⁺: m/z 410.

### Intermediate 42: (6-chloro-5-cyclobutyl-1-(tetrahydro-2H-pyran-2-yl)-1-indazol-4-yl)boronic acid (Int-42)

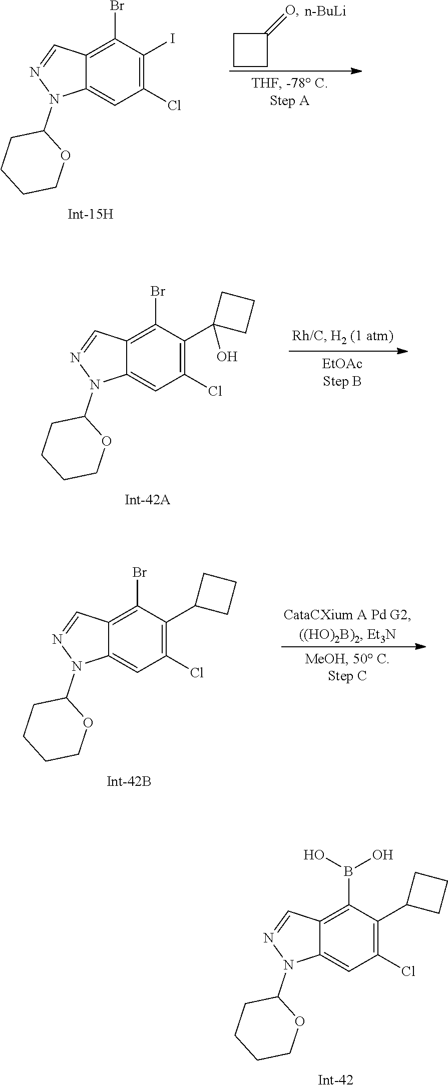

### Step A: 1-(4-bromo-6-chloro-1-(tetrahydro-2H-pyran-2-yl)-1H-indazol-5-yl)cyclobutan-1-ol (Int-42A)

To a solution of 4-bromo-6-chloro-5-iodo-1-(tetrahydro-2H-pyran-2-yl)-1H-indazole (Int-15H) (749 mg, 1.70 mmol) and cyclobutanone (634 μL, 8.49 mmol) in THF (9 mL) was added n-butyllithium (1.8 mL, 2.89 mmol, 1.6 M in hexane) under N₂ atmosphere at −78° C. The resulting mixture was stirred at −78° C. for 30 min. The reaction was quenched with sat. aq. NH₄Cl (10 mL), extracted with EtOAc (60 mL), and washed with brine (15 mL). The organic layer was dried over Na₂SO₄, filtrated, and concentrated in vacuo. The residue was purified by flash silica gel chromatography (0 to 100% EtOAc in hexanes) to provide 1-(4-bromo-6-chloro-1-(tetrahydro-2H-pyran-2-yl)-1H-indazol-5-yl)cyclobutan-1-ol (Int-42A). MS (ESI): [M+H]⁺ m/z 385, 387.

### Step B: 4-bromo-6-chloro-5-cyclobutyl-1-(tetrahydro-2H-pyran-2-yl)-1H-indazole (Int-42B)

A mixture of 1-(4-bromo-6-chloro-1-(tetrahydro-2H-pyran-2-yl)-1H-indazol-5-yl)cyclobutan-1-ol (Int-42A) (45 mg, 0.117 mmol), rhodium on carbon (120 mg, 5 wt %) in EtOAc (2 ml) was degassed and charged with H₂ (3×). The mixture was heated to 50° C. and stirred for 1.5 h. After cooling, the insoluble materials were removed by filtration through a celite pad, which was washed with EtOAc (20 mL). The filtrate was concentrated under reduced pressure to provide 4-bromo-6-chloro-5-cyclobutyl-1-(tetrahydro-2H-pyran-2-yl)-1H-indazole (Int-42B). MS (ESI): [M+H]⁺ m/z 370, 372.

### Step C: (6-chloro-5-cyclobutyl-1-(tetrahydro-2H-pyran-2-yl)-1H-indazol-4-ylboronic acid (Int-42)

A vial was loaded with 4-bromo-6-chloro-5-cyclobutyl-1-(tetrahydro-2H-pyran-2-yl)-1H-indazole (Int-42B) (86.4 mg, 0.234 mmol), hypodiboric acid (62.9 mg, 0.701 mmol), and cataCXium A Pd G2 (7.8 mg, 0.012 mmol). The reaction was dissolved in MeOH (1 mL) and triethylamine (114 μL, 0.818 mmol) under N₂. The mixture was stirred at 50° C. for 1 h under N₂ atmosphere. The mixture was concentrated in vacuo, redissolved in EtOAc, and washed with sat. aq. NH₄Cl. The layers were shaken and separated and the aqueous phase was extracted with EtOAc. The combined organic layers were washed with brine, dried over Na₂SO₄, filtered, and evaporated under reduced pressure to provide (6-chloro-5-cyclobutyl-1-(tetrahydro-2H-pyran-2-yl)-1H-indazol-4-yl)boronic acid (Int-42), which was used directly in the next step without further purification. MS (ESI): [M+H]⁺ m/z 335.

### Intermediate 43: (6-chloro-5-(cyclobut-1-en-1-yl)-1-(tetrahydro-2H-pyran-2-yl)-1H-indazol-4-yl)boronic acid (Int-43)

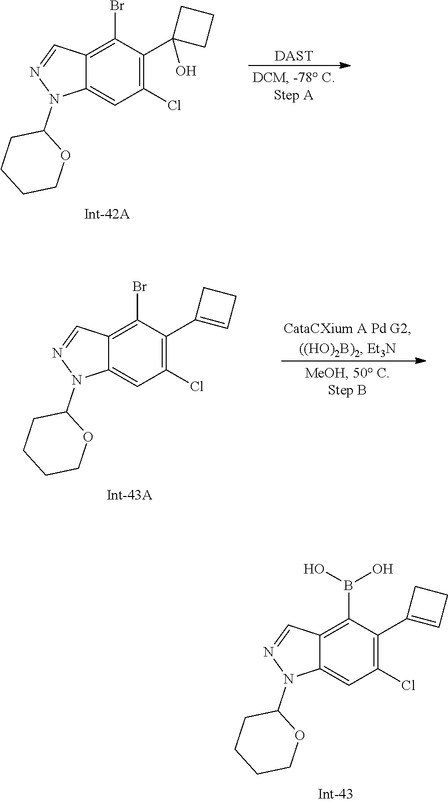

### Step A: 4-bromo-6-chloro-5-(cyclobut-1-en-1-yl)-1-(tetrahydro-2H-pyran-2-yl)-1H-indazole (Int-43A)

To a solution of 1-(4-bromo-6-chloro-1-(tetrahydro-2H-pyran-2-yl)-1H-indazol-5-yl)cyclobutan-1-ol (Int-42A) (220.8 mg, 0.572 mmol) in DCM (5 mL) was added diethylaminosulfur trifluoride (113 μL, 0.859 mmol) at −78° C. The mixture was stirred at −78° C. for 1 h. The mixture was quenched with sat. aq. NaHCO₃ and extracted with EtOAc (3×10 mL). The combined organic layers were washed with brine, dried over MgSO₄, filtered, and concentrated in vacuo. The residue was purified by flash silica gel chromatography (0 to 100% EtOAc in hexanes) to provide 4-bromo-6-chloro-5-(cyclobut-1-en-1-yl)-1-(tetrahydro-2H-pyran-2-yl)-1H-indazole (Int-43A). MS (ESI): [M+H]⁺ m/z 367, 369.

### Step B: (6-chloro-5-(cyclobut-1-en-1-yl)-1-(tetrahydro-2H-pyran-2-yl)-1H-indazol-4-yl)boronic acid (Int-43)

A vial was loaded with 4-bromo-6-chloro-5-(cyclobut-1-en-1-yl)-1-(tetrahydro-2H-pyran-2-yl)-1H-indazole (Int-43A) (20.6 mg, 0.056 mmol), hypodiboric acid (25.1 mg, 0.280 mmol), and cataCXium A Pd G2 (1.9 mg, 2.80 μmol). The reaction was dissolved in MeOH (1 mL) and triethylamine (27 μL, 0.196 mmol) under N₂. The mixture was stirred at 50° C. for 1 h under N₂ atmosphere. The mixture was concentrated in vacuo, redissolved in EtOAc, and washed with sat. aq. NH₄Cl. The layers were shaken and separated and the aqueous phase was extracted with EtOAc. The combined organic layers were washed with brine, dried over Na₂SO₄, filtered, and evaporated under reduced pressure to provide (6-chloro-5-(cyclobut-1-en-1-yl)-1-(tetrahydro-2H-pyran-2-yl)-1H-indazol-4-yl)boronic acid (Int-43), which was used directly in the next step without further purification. MS (ESI): [M+H]⁺ m/z 333.

### Intermediate 44: (6-chloro-5-(1-fluorocyclopropyl)-1-(tetrahydro-2H-pyran-2-yl)-1H-indazol-4-yl)boronic acid (Int-44)

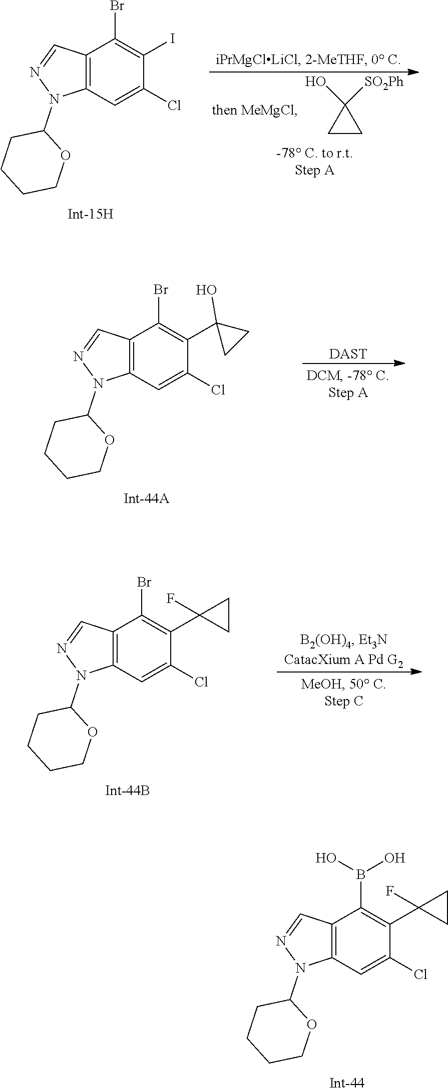

### Step A: 1-(4-bromo-6-chloro-1-(tetrahydro-2H-pyran-2-yl)-1H-indazol-5-yl)cyclopropan-1-ol (Int-44A)

4-bromo-6-chloro-5-iodo-1-(tetrahydro-2H-pyran-2-yl)-1H-indazole (Int-15H) (0.2019 g, 0.457 mmol) was added to a vial with a stir bar and the headspace swept with N₂. 2-methyltetrahydrofuran (880 μL) was added to the vial via syringe and the reaction was cooled to 0° C. Isopropylmagnesium chloride lithium chloride complex (0.343 mL, 0.686 mmol, 2 M in THF) was added dropwise via syringe. The reaction was stirred at 0° C. for 30 min. A separate 50 mL round bottom flask with a stir bar was charged with 1-(phenylsulfonyl)cyclopropan-1-ol (80 mg, 0.387 mmol) and placed under N₂. 2-Me THF (880 μl) was added and the solution was cooled to −78° C. Methylmagnesium chloride (123 μL, 0.368 mmol, 3 M in THF) was added to the reaction followed by the contents of the first reaction flask containing 4-bromo-6-chloro-5-iodo-1-(tetrahydro-2H-pyran-2-yl)-1H-indazole, both dropwise via syringe. The reaction was allowed to warm to room temperature overnight. Sat. aq. Na₂CO₃ (50 mL), H₂O (500 mL), brine (25 mL), and EtOAc (100 mL) were added and the layers were separated. The aqueous phase was extracted with EtOAc (2×150 mL) and the combined organic layers were washed with brine (100 mL), dried over MgSO₄, filtered, and concentrated. The crude residue was purified via silica gel chromatography (0 to 40% EtOAc in hexanes) to provide 1-(4-bromo-6-chloro-1-(tetrahydro-2H-pyran-2-yl)-1H-indazol-5-yl)cyclopropan-1-ol (Int-44A). MS (ESI): [M+H]⁺ m/z 371.

### Step B: 4-bromo-6-chloro-5-(1-fluorocyclopropyl)-1-(tetrahydro-2H-pyran-2-yl)-1H-indazole (Int-44B)

Diethylaminosulfur trifluoride (90 μL, 0.681 mmol) was added to a solution of 1-(4-bromo-6-chloro-1-(tetrahydro-2H-pyran-2-yl)-1H-indazol-5-yl)cyclopropan-1-ol (Int-44A) (111.6 mg, 0.300 mmol) in DCM (2.5 mL) at −78° C. The mixture was stirred at −78° C. for 50 min. The mixture was quenched with sat. aq. NaHCO₃ and extracted with DCM (3×). The combined organic layers were washed with brine, dried over MgSO₄, filtered, and concentrated in vacuo. The crude residue was purified by silica gel chromatography (0 to 40% EtOAc in hexane) to afford 4-bromo-6-chloro-5-(1-fluorocyclopropyl)-1-(tetrahydro-2H-pyran-2-yl)-1H-indazole (Int-44B). MS (ESI): [M+H]⁺ m/z 373, 375.

### Step C: (6-chloro-5-(1-fluorocyclopropyl)-1-(tetrahydro-2H-pyran-2-yl)-1H-indazol-4-yl)boronic acid (Int-44)

CataCXium A Pd G2 (24 mg, 0.036 mmol), hypodiboric acid (56.4 mg, 0.629 mmol), and Et₃N (110 μL, 0.789 mmol) in MeOH (2000 μl) were added to a vial containing 4-bromo-6-chloro-5-(1-fluorocyclopropyl)-1-(tetrahydro-2H-pyran-2-yl)-1H-indazole (Int-44B) (78.4 mg, 0.210 mmol). The mixture was evacuated and backfilled with N₂ (3×). The mixture was stirred at 50° C. for 2 h. The mixture was diluted with water and extracted with EtOAc (3×) and the combined organic layers were washed with brine, dried over MgSO₄, filtered, and concentrated in vacuo to afford (6-chloro-5-(1-fluorocyclopropyl)-1-(tetrahydro-2H-pyran-2-yl)-1H-indazol-4-yl)boronic acid (Int-44). MS (ESI): [M+H]⁺ m/z 339.

The compound in the table below was synthesized using a similar procedure as described in the synthesis of Int-44 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-45 | 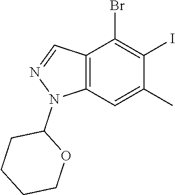 [SMILES: Cc1cc2c(cnn2C2CCCCO2)c(Br)c1I] Int-4C | 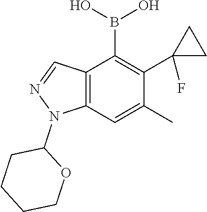 [SMILES: Cc1cc2c(cnn2C2CCCCO2)c(B(O)O)c1C1(F)CC1] | (5-(1-fluorocyclopropyl)-6- methyl-1-(tetrahydro-2H- pyran-2-yl)-1H-indazol-4- yl)boronic acid | 319 |

### Intermediate 46: Dimethyl(5-(tert-butyl)-6-chloro-1-(tetrahydro-2H-pyran-2-yl)-1H-indazol-4-yl) boronate (Int-46)

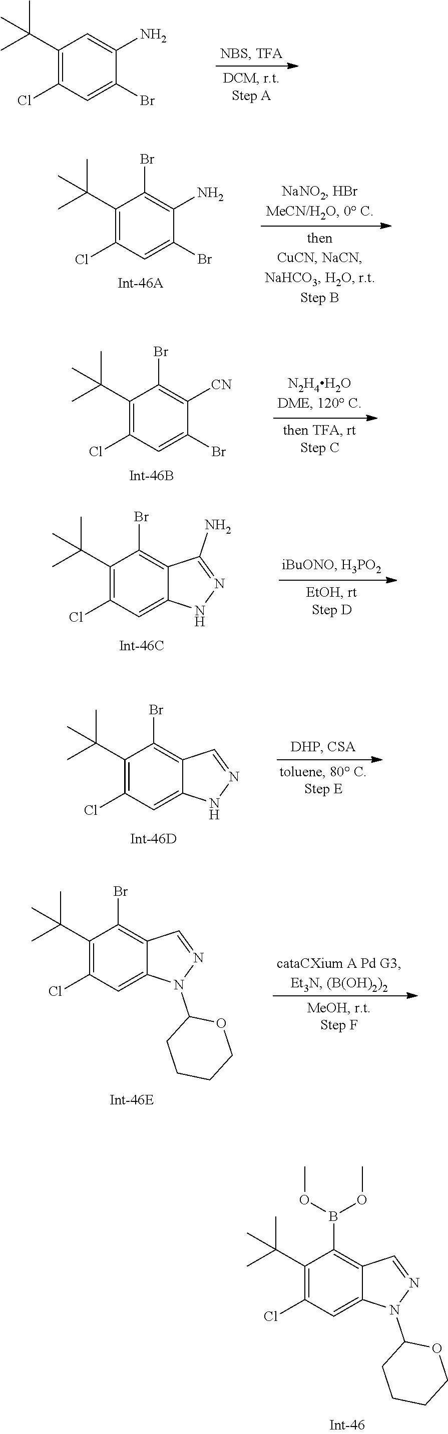

### Step A: 2,6-dibromo-3-(tert-butyl)-4-chloroaniline (Int-46A)

NBS (6.6 g, 37 mmol) was added to a solution of 2-bromo-5-(tert-butyl)-4-chloroaniline (8.9 g, 34 mmol) in DCM (90 mL) and TFA (4.5 mL, 61 mmol) at room temperature. After stirring at room temperature for 2 h, the mixture was diluted with CHCl₃ and washed with sat. aq. NaHCO₃ and brine. The organic layer was dried over Na₂SO₄, filtered, and evaporated. The crude residue was purified by silica gel chromatography (hexane/CHCl₃) to afford 2,6-dibromo-3-(tert-butyl)-4-chloroaniline (Int-46A). MS (ESI): m/z (M+H)⁺ 340, 342, 344.

### Step B: 2,6-dibromo-3-(tert-butyl)-4-chlorobenzonitrile (Int-46B)

A solution of NaNO₂ (976 mg, 14.1 mmol) in H₂O (1 mL) was added to a solution of 2,6-dibromo-3-(tert-butyl)-4-chloroaniline (Int-46A) (4.20 g, 12.3 mmol) in CH₃CN (30 mL), H₂O (13.0 mL), and HBr (9.87 mL, 84.9 mmol) at 0° C. After stirring at 0° C. for 30 min, the reaction mixture was added slowly dropwise to a solution of NaCN (1.21 g, 24.6 mmol), CuCN (1.10 g, 12.3 mmol), and NaHCO₃ (5.17 g, 61.5 mmol) in H₂O (30 mL). After stirring at room temperature for 1 h, the reaction was diluted with EtOAc and filtered through CELITE®. The filtrate was washed with brine, dried over Na₂SO₄, filtered, and evaporated. The crude residue was purified by silica gel chromatography (hexane/CHCl₃) to afford 2,6-dibromo-3-(tert-butyl)-4-chlorobenzonitrile (Int-46B). ¹H NMR (400 MHz, CDCl₃) δ 7.69 (s, 1H), 1.71 (s, 9H).

### Step C: 4-bromo-5-(ter-butyl)-6-chloro-1H-indazol-3-amine (Int-46C)

Hydrazine hydrate (1.72 mL, 34.7 mmol) was added to a solution of 2,6-dibromo-3-(tert-butyl)-4-chlorobenzonitrile (Int-46B) (2.03 g, 5.78 mmol) in DME (30 mL) at room temperature. The reaction mixture was stirred at 120° C. using microwave for 8 h. After cooling to room temperature, TFA (5.15 mL, 69.3 mmol) was added to the mixture. After stirring for 30 min, the mixture was diluted with EtOAc, washed with sat. aq. NaHCO₃ and brine. The organic layer was dried over Na₂SO₄, filtered, and evaporated. The crude residue was purified by silica gel chromatography (hexane/CHCl₃) to afford 4-bromo-5-(tert-butyl)-6-chloro-1H-indazol-3-amine (Int-46C). MS (ESI): m/z (M+H)⁺ 302, 304. ¹H NMR (400 MHz, CDCl₃) δ 7.33 (s, 1H), 1.75 (s, 9H).

### Step D: 4-bromo-5-(ter-butyl)-6-chloro-1H-indazole (Int-46D)

Isobutyl nitrite (0.120 mL, 1.01 mmol) was added to a solution of 4-bromo-5-(tert-butyl)-6-chloro-1H-indazol-3-amine (Int-46C) (102 mg, 0.337 mmol) in EtOH (2.0 mL) and H₃PO₂ (0.280 mL, 2.70 mmol) at 0° C. After warming to room temperature and stirring for 2 h, the mixture was diluted with EtOAc, washed with brine, dried over Na₂SO₄, filtered, and evaporated. The crude residue was purified by silica gel chromatography (hexane/EtOAc) to afford 4-bromo-5-(tert-butyl)-6-chloro-1H-indazole (Int-46D). MS (ESI): m/z (M+H)⁺ 287, 289.

### Step E: 4-bromo-5-(tert-butyl)-6-chloro-1-(tetrahydro-2H-pyran-2-yl)-1H-indazole (Int-46E)

3,4-dihydro-2H-pyran (30 μL, 0.33 mmol) and (+)-CSA (12 mg, 0.050 mmol) were added to a solution of 4-bromo-5-(tert-butyl)-6-chloro-1H-indazole (Int-46D) (48 mg, 0.17 mmol) in toluene (0.8 mL). The mixture was stirred at 80° C. for 1 h. The solution was cooled to room temperature and diluted with EtOAc. After washing with sat. aq. NaHCO₃ and brine, the organic layer was dried over Na₂SO₄, filtered, and evaporated. The crude residue was purified by silica gel chromatography (hexane/EtOAc) to afford 4-bromo-5-(tert-butyl)-6-chloro-1-(tetrahydro-2H-pyran-2-yl)-1H-indazole (Int-46E). MS (ESI): m/z (M+H)⁺ 371, 373.

### Step F: dimethyl(5-(tert-butyl)-6-chloro-1-(tetrahydro-2H-pyran-2-yl)-1H-indazol-4-yl) boronate (Int-46)

Tetrabydroxydiboron (19 mg, 0.22 mmol), cataCXium A Pd G3 (4.4 mg, 0.0060 mmol), and Et₃N (43 μL, 0.309 mmol) were added to a solution of 4-bromo-5-(tert-butyl)-6-chloro-1-(tetrahydro-2H-pyran-2-yl)-1H-indazole (Int-46E) (32 mg, 0.086 mmol) in MeOH (0.86 mL). After stirring at room temperature for 1 h, the solution was filtered through CELITE® and evaporated. The crude residue was purified by silica gel chromatography (hexane/EtOAc) to afford dimethyl(5-(tert-butyl)-6-chloro-1-(tetrahydro-2H-pyran-2-yl)-1H-indazol-4-yl) boronate (Int-45). MS (ESI): m/z (M+H)⁺ 365, 367.

### Intermediate 47: (5-(bicyclo[1.1.1]pentan-1-yl)-1-(tetrahydro-2H-pyran-2-yl)-1H-indazol-4-yl)boronic acid (Int-47)

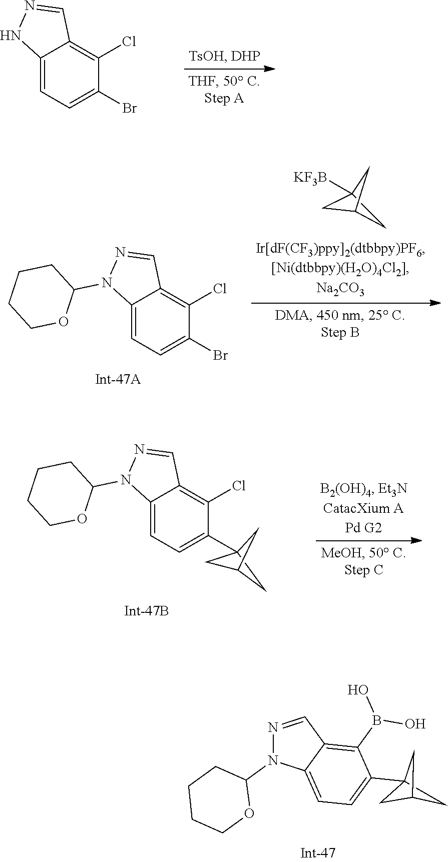

### Step A: 5-bromo-4-chloro-1-(tetrahydro-2H-pyran-2-yl)-1H-indazole (Int-47A)

To a solution of 5-bromo-4-chloro-1H-indazole (800 mg, 3.46 mmol) in THF (10 mL) was added 4-methylbenzenesulfonic acid (59.5 mg, 0.346 mmol) and DHP (0.948 mL, 10.37 mmol). The mixture was stirred at 50° C. for 16 h. The reaction mixture was concentrated in vacuo and the residue was purified by flash silica gel chromatography (0 to 25% EtOAc in petroleum ether) to give 5-bromo-4-chloro-1-(tetrahydro-2H-pyran-2-yl)-1H-indazole (Int-47A). MS (ESI) [M+H]⁺: m/z 315, 317.

### Step B: 5-(bicyclo[1.1.1]pentan-1-yl)-4-chloro-1-(tetrahydro-2H-pyran-2-yl)-1H-indazole (Int-47B)

To a solution of 5-bromo-4-chloro-1-(tetrahydro-2H-pyran-2-yl)-1H-indazole (Int-47A) (200 mg, 0.634 mmol) in DMA (5 mL) was added Na₂CO₃ (269 mg, 2.53 mmol), potassium bicyclo[1.1.1]pentan-1-yltrifluoroborate (333 mg, 1.901 mmol), [Ni(dtbbpy)(H₂O)₄Cl₂](44.7 mg, 0.095 mmol) and (Ir[dF(CF₃)ppy]₂(dtbpy)PF₆ (71.1 mg, 0.063 mmol) at 25° C., and the mixture was stirred at 25° C. for 4 h. The mixture was scaled with a cap and placed in a Merck Photoreactor (450 nm light source, 100% intensity, 1000 rpm stirring and 10000 rpm fan speed) for 4 h. The reaction mixture was diluted with EtOAc (40 mL) and the mixture was washed with brine (3×5 mL). The organic phase was dried over Na₂SO₄, filtered, and concentrated in vacuo. The residue was purified by flash silica gel chromatography (petroleum ether/EtOAc=5/1), followed by preparative HPLC (MeCN/H₂O w/TFA modifier) to give 5-(bicyclo[1.1.1]pentan-1-yl)-4-chloro-1-(tetrahydro-2H-pyran-2-yl)-1H-indazole (Int-47B). MS (ESI) [M+H]⁺: m/z 303.

### Step C: (S-(bicyclo[1.1.1]pentan-1-yl)-1-(tetrahydro-2H-pyran-2-yl)-1H-indazol-4-yl)boronic acid (Int-47)

To a solution of 5-(bicyclo[1.1.1]pentan-1-yl)-4-chloro-1-(tetrahydro-2H-pyran-2-yl)-1H-indazole (Int-47B) (80 mg, 0.264 mmol) in MeOH (1.5 mL) was added Et₃N (0.110 mL, 0.793 mmol), hypodiboric acid (95 mg, 1.057 mmol), and cataCxium A Pd G2 (8.83 mg, 0.013 mmol) at 25° C., and the reaction mixture was stirred at 50° C. for 2 h under N₂. The reaction mixture was concentrated in vacuo and the residue was directly purified by preparative TLC plate (SiO₂, petroleum ether/EtOAc=3/1) to give (5-(bicyclo[1.1.1]pentan-1-yl)-1-(tetrahydro-2H-pyran-2-yl)-1H-indazol-4-yl)boronic acid (Int-47). MS (ESI) [M+H]⁺: m/z 313.

The compound in the table below was synthesized using a similar procedure as described in the synthesis of Int-47 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-48 | 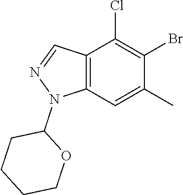 [SMILES: Cc1cc2c(cnn2C2CCCCO2)c(Cl)c1Br] Int-9 | 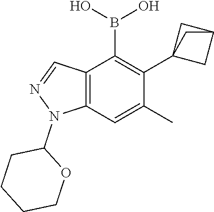 [SMILES: Cc1cc2c(cnn2C2CCCCO2)c(B(O)O)c1C12CC(C1)C2] | (5-(bicyclo[1.1.1] pentan-1-yl)- 6-methyl-1- (tetrahydro-2H- pyran-2-yl)- 1H-indazol-4- yl)boronic acid | 327 |

### Intermediate 49: (5-(1-fluorocyclopropyl)-1-(tetrahydro-2H-pyran-2-yl)-6-(trifluoromethyl)-1H-indazol-4-yl)boronic acid (Int-49)

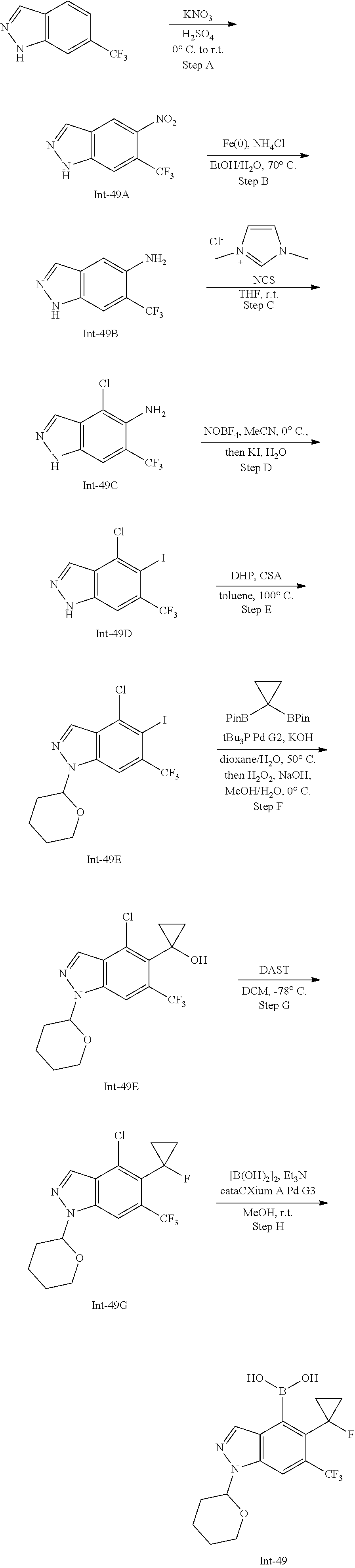

### Step A: 5-nitro-6-(trifluoromethyl)-1H-indazole (Int-49A)

To a solution of 6-(trifluoromethyl)-1H-indazole (7.44 g, 40 mmol) in H₂SO₄ (100 mL, conc. aq.) at 0° C., was slowly added KNO₃ (4.45 g, 44 mmol). The reaction mixture was warmed up to room temperature. After stirring for 3 h at room temperature, it was poured into crushed ice. The precipitated solid was collected by filtration and washed with water to afford 5-nitro-6-(trifluoromethyl)-1H-indazole (Int-49A). MS (ESI) [M+H]⁺: m/z 232.

### Step B: 6-(trifluoromethyl)-1H-indazol-5-amine (Int-49B)

A mixture of 5-nitro-6-(trifluoromethyl)-1H-indazole (Int-49A) (8.91 g, 38.5 mmol), iron (10.8 g, 193 mmol), and NH₄Cl (10.3 g, 193 mmol) in EtOH (120 mL) and water (20 mL) was vigorously stirred at 70° C. for 1 h. The mixture was diluted with EtOAc (˜200 mL), filtered by glass fiber membrane filter, and washed with EtOAc. The filtrate was concentrated under reduced pressure and sat. aq. NaHCO₃ and EtOAc were added. The layers were separated, and the organic phase was washed with brine, dried over Na₂SO₄, filtered, and concentrated to give 6-(trifluoromethyl)-1H-indazol-5-amine (Int-49B). MS (ESI) [M+H]⁺: m/z 202.

### Step C: 4-chloro-6-(trifluoromethyl)-1H-indazol-5-amine (Int-49C)

To a solution of 6-(trifluoromethyl)-1H-indazol-5-amine (Int-49B) (616 mg, 3.06 mmol) in THF (15 mL) was added 1,3-dimethylimidazolium chloride (40 mg, 0.302 mmol) and NCS (430 mg, 3.22 mmol). After stirred overnight at room temperature, sat. aq. NaHCO₃ and EtOAc were added to the reaction mixture. The layers were separated, and the organic phase was washed with brine, dried over Na₂SO₄, filtered, and concentrated. The residue was purified by silica gel chromatography (0 to 100% EtOAc in hexanes) to give 4-chloro-6-(trifluoromethyl)-1H-indazol-5-amine (Int-49C). MS (ESI) [M+H]⁺: m/z 236.

### Step D: 4-chloro-5-iodo-6-(trifluoromethyl)-1H-indazole (Int-49D)

To a solution of 4-chloro-6-(trifluoromethyl)-1H-indazol-5-amine (Int-49C) (634 mg, 2.69 mmol) in MeCN (20 mL) was added nitrosyl tetrafluoroborate (380 mg, 3.25 mmol) at 0° C. The mixture was stirred at 0° C. for 10 min before a solution of KI (5.0 g, 30 mmol) in water (10 mL) was added with vigorous stirring. After stirring for 10 min, EtOAc and water were added and the layers were separated. The organic phase was washed with the mixture of sat. aq. NaHCO₃ and Na₂S₂O₃ solution, dried over Na₂SO₄, and concentrated to give 4-chloro-5-iodo-6-(trifluoromethyl)-1H-indazole (Int-49D). MS (ESI) [M+H]⁺: m/z 347.

### Step E: 4-chloro-5-iodo-1-(tetrahydro-2H-pyran-2-yl)-6-(trifluoromethyl)-1H-indazole (Int-49E)

A mixture of 4-chloro-5-iodo-6-(trifluoromethyl)-1H-indazole (Int-49D) (920 mg), (1R)-(−)-camphor-10-sulfonic acid (60 mg, 0.258 mmol), and 3,4-dihydro-2H-pyran (0.72 mL, 8.0 mmol) in toluene (20 mL) was stirred at 100° C. for 2.5 h. The reaction mixture was cooled to room temperature, and sat. aq. NaHCO₃ was added. The layers were separated, and the organic phase was washed with brine, dried over Na₂SO₄, filtered, and concentrated. The residue was purified by silica gel chromatography (0 to 100% EtOAc in hexanes) to give 4-chloro-5-iodo-1-(tetrahydro-2H-pyran-2-yl)-6-(trifluoromethyl)-1H-indazole (Int-49E). MS (ESD) [M+H]⁺: m/z 431.

### Step F: 1-(4-chloro-1-(tetrahydro-2H-pyran-2-yl)-6-(trifluoromethyl)-1H-indazol-5-yl)cyclopropan-1-ol (Int-49F)

KOH (1.25 mL, 2.50 mmol, 2N in H₂O) was added to a solution of (4-chloro-5-iodo-1-(tetrahydro-2H-pyran-2-yl)-6-(trifluoromethyl)-1H-indazole (Int-49E) (430 mg, 0.999 mmol), 2,2′-cyclopropylidenebis(4,4,5,5-tetramethyl-1,3,2-dioxaborolane) (740 mg, 2.517 mmol), and chloro[(tri-tert-butylphosphine)-2-(2-aminobiphenyl)]palladium (II) (100 mg, 0.195 mmol) in 1,4-dioxane (10 mL). The mixture was stirred at 50° C. for 3 h. The mixture was cooled to 0° C., and MeOH (1 mL) and NaOH (5 mL, 2N in H₂O) were added. H₂O₂ (1.2 mL, 30 wt % in H₂O) was added slowly at 0° C. and stirred for 10 min. After 10 min, MeOH (2 mL) was added and stirred at 0° C. for 30 min. The reaction was quenched with H₃PO₄ (10% aq.) and CHCl₃ and the layers were separated. The organic phase was washed with brine, dried over Na₂SO₄, filtered, and concentrated. The residue was purified by silica gel chromatography (0 to 100% EtOAc in hexanes) to afford 1-(4-chloro-1-(tetrahydro-2H-pyran-2-yl)-6-(trifluoromethyl)-1H-indazol-5-yl)cyclopropan-1-ol (Int-49F). MS (ESI) [M+H]⁺: m/z 361.

### Step G: 4-chloro-5-(1-fluorocyclopropyl)-1-(tetrahydro-2H-pyran-2-yl)-6-(trifluoromethyl)-1H-indazole (Int-49G)

Diethylaminosulfur trifluoride (0.035 mL, 0.265 mmol) was added to a solution of 1-(4-chloro-1-(tetrahydro-2H-pyran-2-yl)-6-(trifluoromethyl)-1H-indazol-5-yl)cyclopropan-1-ol (Int-49F) (43 mg, 0.119 mmol) in DCM (3 mL) at −78° C. After stirring at −78° C. for 30 min, sat. aq. NaHCO₃ and EtOAc were added to the reaction mixture. The layers were separated and the organic phase was washed with brine, dried over Na₂SO₄, filtered, and concentrated. The residue was purified by silica gel chromatography (0 to 100% EtOAc in hexanes) to give 4-chloro-5-(1-fluorocyclopropyl)-1-(tetrahydro-2H-pyran-2-yl)-6-(trifluoromethyl)-1H-indazole (Int-49G). MS (ESI) [M+H]⁺: m/z 363.

### Step H: (5-(1-fluorocyclopropyl)-1-(tetrahydro-2H-pyran-2-yl)-6-(trifluoromethyl)-1H-indazol-4-yl)boronic acid (Int-49)

The mixture of 4-chloro-5-(1-fluorocyclopropyl)-1-(tetrahydro-2H-pyran-2-yl)-6-(trifluoromethyl)-1H-indazole (Int-49G) (34 mg, 0.0937 mmol), tetrahydroxydiboron (40 mg, 0.446 mmol), cataCXium A Pd G3 (6 mg, 0.0082 mmol) and Et₃N (0.080 ml, 0.574 mmol) in MeOH (1 mL) was stirred at room temperature for 60 hours. EtOAc (30 mL), CHCl₃ (1 mL), and H₃PO₄ (10% aq.) were added to the reaction mixture. The layers were separated and the organic phase was washed with brine, dried over Na₂SO₄, filtered, and concentrated. The residue was purified by silica gel chromatography (0 to 100% EtOAc in hexanes) to give (5-(1-fluorocyclopropyl)-1-(tetrahydro-2H-pyran-2-yl)-6-(trifluoromethyl)-1H-indazol-4-yl)boronic acid (Int-49). MS (ESI) [M+H]⁺: m/z 373.

### Intermediate 50: (5-cyclopropyl-1-(tetrahydro-2H-pyran-2-yl)-6-(trifluoromethyl)-1H-indazol-4-yl)boronic acid (Int-50)

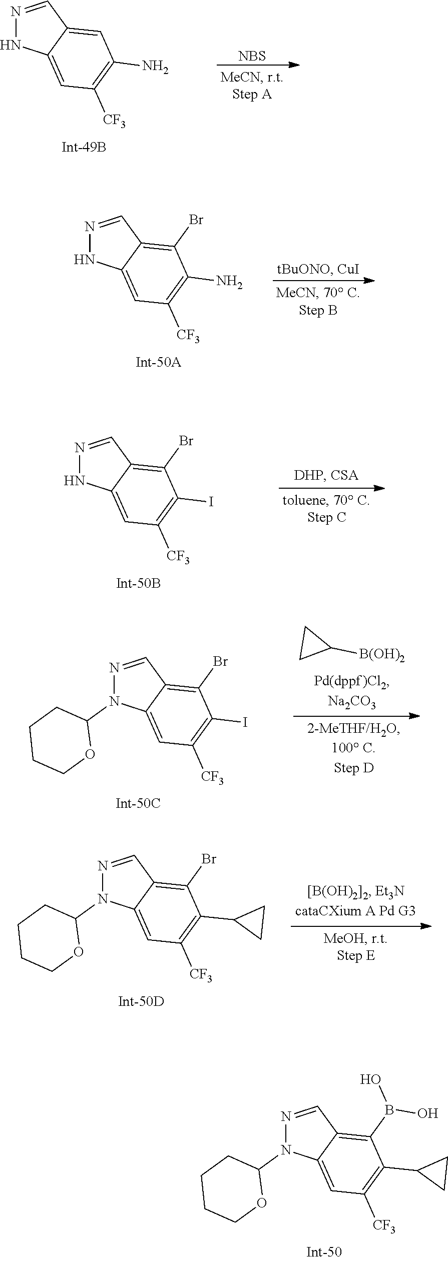

### Step A: 4-bromo-6-(trifluoromethyl)-1H-indazol-5-amine (Int-50A)

NBS (880 mg, 5.0 mmol) was added to a stirred solution of 6-(trifluoromethyl)-1H-indazol-5-amine (Int-49B) (1.0 g, 5.0 mmol) in MeCN (25 mL) at room temperature. After 10 min, the mixture was partitioned between EtOAc and sat. aq. NaHCO₃. The organic phase was washed with brine, dried over Na₂SO₄, and concentrated. The crude residue was purified via silica gel chromatography (0 to 20% EtOAc in hexane) to give 4-bromo-6-(trifluoromethyl)-1H-indazol-5-amine (Int-50A). MS (ESI): m/z (M+H)⁺ 280.

### Step B: 4-bromo-5-iodo-6-(trifluoromethyl)-1H-indazole (Int-50B)

Copper iodide (612 mg, 3.21 mmol) and tert-butyl nitrite (0.395 ml, 3.32 mmol) were added to a stirred solution of 4-bromo-6-(trifluoromethyl)-1H-indazol-5-amine (Int-50A) (300 mg, 1.07 mmol) in MeCN (25 mL) at room temperature. The mixture was warmed to 70° C. and stirred for 30 min. After cooling, the mixture was partitioned between EtOAc and sat. aq. NaHCO₃. The organic phase was washed with brine, dried over Na₂SO₄, and concentrated. The crude residue was purified via silica gel chromatography (0 to 20% EtOAc in hexane) to give 4-bromo-5-iodo-6-(trifluoromethyl)-1H-indazole (Int-50B). MS (ESI): m/z (M+H)⁺ 391.

### Step C: 4-bromo-5-iodo-1-(tetrahydro-2H-pyran-2-yl)-6-(trifluoromethyl)-1H-indazole (Int-50C)

A mixture of 4-bromo-5-iodo-6-(trifluoromethyl)-1H-indazole (Int-50B) (318 mg, 0.812 mmol), 3,4-dihydro-2H-pyran (0.15 mL, 1.61 mmol), (1R)-(−)-camphor-10-sulfonic acid (24 mg, 0.107 mmol) in THF (10 mL) was stirred at 70° C. for 3 h. After cooling to room temperature, the mixture was partitioned between EtOAc and aqueous sat. aq. NaHCO₃. The organic phase was washed with brine, dried over Na₂SO₄, filtered, and concentrated. The crude residue was purified via silica gel chromatography (0 to 10% EtOAc in hexane) to give 4-bromo-5-iodo-1-(tetrahydro-2H-pyran-2-yl)-6-(trifluoromethyl)-1H-indazole (Int-50C). MS (ESI): m/z (M+H)⁺ 475.

### Step D: 4-bromo-5-cyclopropyl-1-(tetrahydro-2H-pyran-2-yl)-6-(trifluoromethyl)-1H-indazole (Int-50D)

A mixture of 4-bromo-5-iodo-1-(tetrahydro-2H-pyran-2-yl)-6-(trifluoromethyl)-1H-indazole (Int-50C) (170 mg, 0.358 mmol), cyclopropylboronic acid (31 mg, 0.358 mmol), [1,1′-bis(diphenylphosphino)ferrocene]dichloropalladium (II) (52 mg, 0.071 mmol), sodium carbonate (1.07 mL, 1.07 mmol, 1 M in H₂O) in 2-methyltetrahydrofuran (3.6 mL) was evacuated and backfilled with N₂ (3×). The mixture was stirred at 100° C. under N₂ for 8 h. Additional cyclopropylboronic acid (15 mg, 0.179 mmol) and [1,1′-bis(diphenylphosphino)ferrocene]dichloropalladium (II) (26 mg, 0.036 mmol) were added to the mixture and stirred at 100° C. for 2 h. After cooling to room temperature, the mixture was partitioned between EtOAc and water. The organic phase was washed with brine, dried over Na₂SO₄, filtered, and concentrated. The crude residue was purified via silica gel chromatography (0 to 10% EtOAc in hexane) to give 4-bromo-5-cyclopropyl-1-(tetrahydro-2H-pyran-2-yl)-6-(trifluoromethyl)-1H-indazole (Int-50D). MS (ESI): m/z (M+H)⁺ 389.

### Step E: (5-cyclopropyl-1-(tetrahydro-2H-pyran-2-yl)-6-(trifluoromethyl)-1H-indazol-4-yl)boronic acid (Int-50)

CataCXium A Pd G3 (12 mg, 0.017 mmol) was added to a stirred mixture of 4-bromo-5-cyclopropyl-1-(tetrahydro-2H-pyran-2-yl)-6-(trifluoromethyl)-1H-indazole (Int-50D) (66 mg, 0.170 mmol), triethylamine (0.095 mL, 0.680 mmol), and tetrahydroxydiboron (31 mg, 0.340 mmol) in MeOH (3.4 mL) at room temperature. The reaction was stirred at room temperature for 1 h. The mixture was filtered through a pad of CELITE®, washed with MeOH and the filtrate was concentrated. The residue was purified via silica gel chromatography (10 to 50% EtOAc in hexanes) to give (5-cyclopropyl-1-(tetrahydro-2H-pyran-2-yl)-6-(trifluoromethyl)-1H-indazol-4-yl)boronic acid (Int-50). MS (ESI): m/z (M+H)⁺ 355.

### Intermediate 51: (5-(1-fluorovinyl)-1-(tetrahydro-2H-pyran-2-yl)-6-(trifluoromethyl)-1H-indazol-4-yl)boronic acid (Int-51)

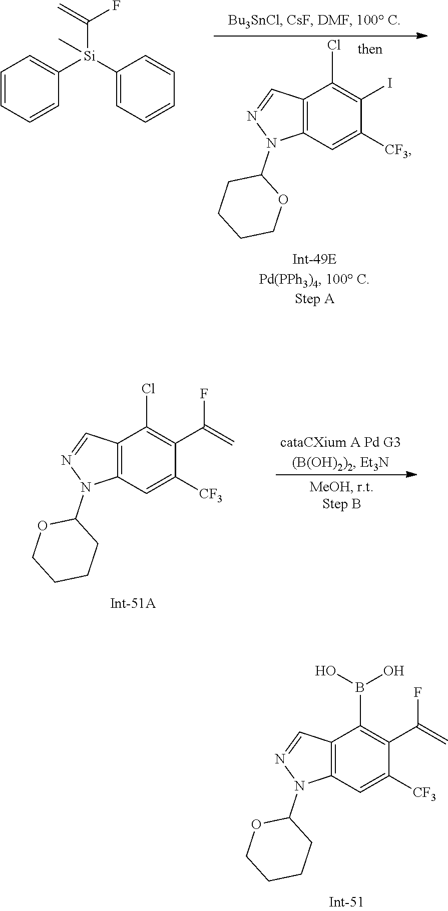 [SMILES: C=C(F)[Si](C)(c1ccccc1)c1ccccc1 ; FC(F)(F)c1cc2c(cnn2C2CCCCO2)c(Cl)c1I ; C=C(F)c1c(C(F)(F)F)cc2c(cnn2C2CCCCO2)c1Cl ; "cataCXium A Pd G3(B(OH)2)2, Et3N" ; C=C(F)c1c(C(F)(F)F)cc2c(cnn2C2CCCCO2)c1B(O)O]

### Step A: 4-chloro-5-(1-fluorovinyl)-1-(tetrahydro-2H-pyran-2-yl)-6-(trifluoromethyl)-1H-indazole (Int-51A)

A mixture of (1-fluorovinyl)methyldiphenylsilane (0.200 ml, 0.73 mmol), tributyltin chloride (0.200 ml, 0.737 mmol), and cesium fluoride (120 mg, 0.78997339 mmol) in DMF (1 mL) was stirred at 100° C. overnight. Then 4-chloro-5-iodo-1-(tetrahydro-2H-pyran-2-yl)-6-(trifluoromethyl)-1H-indazole (Int-49E) (264 mg, 0.613 mmol) and tetrakis(triphenylphosphine) palladium (0) (0.035 g, 0.030 mmol) were added to the reaction mixture and the reaction was stirred at 100° C. for 6 h. After the reaction was cooled to room temperature, the mixture was purified directly via NH-silica gel chromatography (0 to 100% EtOAc in hexanes) to give 4-chloro-5-(1-fluorovinyl)-1-(tetrahydro-2H-pyran-2-yl)-6-(trifluoromethyl)-1H-indazole (Int-51A). MS (ESI) [M+H]⁺: m/z 349.

### Step B: (5-(1-fluorovinyl)-1-(tetrahydro-2H-pyran-2-yl)-6-(trifluoromethyl)-1H-indazol-4-yl)boronic acid (Int-51)

4-chloro-5-(1-fluorovinyl)-1-(tetrahydro-2H-pyran-2-yl)-6-(trifluoromethyl)-1H-indazole (Int-51A) (228 mg, 0.635 mmol), tetrahydroxydiboron (240 mg, 2.68 mmol), cataCXium A Pd G3 (40 mg, 0.055 mmol), Et₃N (0.55 ml, 4.0 mmol), and MeOH (3 ml) were stirred at room temperature overnight. EtOAc, CHCl₃, and H₃PO₄ (10% aq.) were added to the reaction. The organic layer was separated, washed with brine, dried over Na₂SO₄, and concentrated. The crude residue was purified by silica gel chromatography (CHCl₃-ethanol) to give (5-(1-fluorovinyl)-1-(tetrahydro-2H-pyran-2-yl)-6-(trifluoromethyl)-1H-indazol-4-yl)boronic acid (Int-51). MS (ESI) [M+H]⁺: m/z 359.

### Intermediate 52: (5-(difluoromethyl)-1-(tetrahydro-2H-pyran-2-yl)-6-(trifluoromethyl)-1H-indazol-4-yl)boronic acid (Int-52)

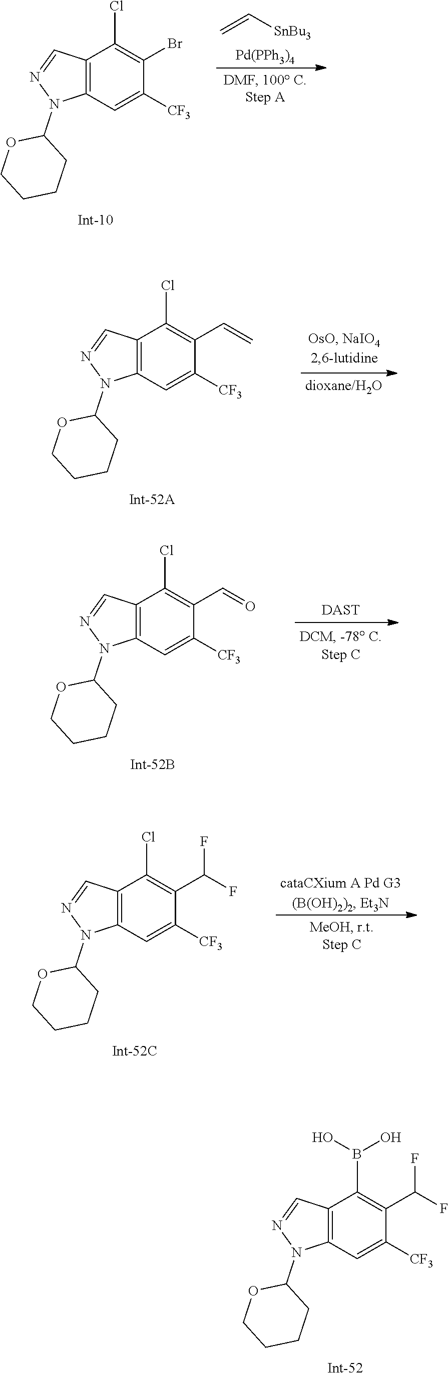

### Step A: 4-chloro-1-(tetrahydro-2H-pyran-2-yl)-6-(trifluoromethyl)-5-vinyl-1H-indazole (Int-52A)

5-bromo-4-chloro-1-(tetrahydro-2H-pyran-2-yl)-6-(trifluoromethyl)-1H-indazole (Int-10) (384 mg, 1.00 mmol), tetrakis(triphenylphosphine) palladium (0) (60 mg, 0.0519 mmol), and tributyl(vinyl) stannane (0.320 mL, 1.10 mmol) in DMF (2 mL) was stirred at 100° C. overnight. After the reaction was cooled to room temperature, the mixture was charged on an amino-SiO₂ column for injection and purified by silica gel chromatography (hexane-EtOAc) to give 4-chloro-1-(tetrahydro-2H-pyran-2-yl)-6-(trifluoromethyl)-5-vinyl-1H-indazole (Int-52A). MS (ESI) [M+H]⁺: m/z 331.

### Step B: 4-chloro-1-(tetrahydro-2H-pyran-2-yl)-6-(trifluoromethyl)-1H-indazole-5-carbaldehyde (Int-52B)

2,6-lutidine (0.240 mL, 2.07 mmol), osmium tetroxide (0.260 mL, 0.0102 mmol, 1 wt % in H₂O), and sodium periodate (880 mg, 4.11 mmol) were added to a solution of 4-chloro-1-(tetrahydro-2H-pyran-2-yl)-6-(trifluoromethyl)-5-vinyl-1H-indazole (Int-52A) (340 mg, 1.02 mmol) in 1,4-dioxane (9.0 mL) and water (3.0 mL). The mixture was stirred at room temperature overnight. Additional sodium periodate (440 mg) and osmium tetroxide (0.260 mL, 0.0102 mmol, 1 wt % in H₂O) were added after this time. After stirring at room temperature overnight, the reaction mixture was diluted with sat. aq. NaHCO₃ and EtOAc. The organic layer was separated, washed with brine, and dried over Na₂SO₄. The mixture was filtered and concentrated under reduced pressure. The residue obtained was purified by silica gel chromatography (hexane/EtOAc=100/0 to 50/50) to afford 4-chloro-1-(tetrahydro-2H-pyran-2-yl)-6-(trifluoromethyl)-1H-indazole-5-carbaldehyde (Int-52B). MS (ESI) [M+H]⁺: m/z 333.

### Step C: 4-chloro-5-(difluoromethyl)-1-(tetrahydro-2H-pyran-2-yl)-6-(trifluoromethyl)-1H-indazole (Int-52C)

Diethylaminosulfur trifluoride (0.060 mL, 0.454 mmol) was added to a solution of 4-chloro-1-(tetrahydro-2H-pyran-2-yl)-6-(trifluoromethyl)-1H-indazole-5-carbaldehyde (Int-52C) (46 mg, 0.138 mmol) in DCM (3.0 mL) at −78° C. The mixture was warmed to room temperature and was stirred for 3 days. The reaction mixture was quenched with sat. aq. NaHCO₃ and diluted with EtOAc. The organic layer was separated, washed with brine, and dried over Na₂SO₄. The mixture was filtered and the filtrate was concentrated under reduced pressure. The residue obtained was purified by silica gel chromatography (hexane/EtOAc=97/3 to 80/20) to afford 4-chloro-5-(difluoromethyl)-1-(tetrahydro-2H-pyran-2-yl)-6-(trifluoromethyl)-1H-indazole (Int-52C). MS (ESI) [M+H]⁺: m/z 355.

### Step D: (5-(difluoromethyl)-1-(tetrahydro-2H-pyran-2-yl)-6-(trifluoromethyl)-1H-indazol-4-yl)boronic acid (Int-52)

Tetrahydroxydiboron (100 mg, 1.12 mmol), triethylamine (0.24 mL, 1.7 mmol), and cataCXium A Pd G3 (0.015 g, 0.0206 mmol) were added to a solution of 4-chloro-5-(difluoromethyl)-1-(tetrahydro-2H-pyran-2-yl)-6-(trifluoromethyl)-1H-indazole (Int-52C) (98 mg, 0.276 mmol) in MeOH (3 mL). The mixture was stirred at room temperature for 3 h under N₂. After 3 h, the reaction was diluted with H₃PO₄ (10% aq.) and CHCl₃. The organic layer was separated, washed with brine, dried with anhydrous Na₂SO₄, and concentrated. The residue was purified by flash silica gel chromatography (hexane-EtOAc/ethanol (4/1)) to afford (5-(difluoromethyl)-1-(tetrahydro-2H-pyran-2-yl)-6-(trifluoromethyl)-1H-indazol-4-yl)boronic acid (Int-52). MS (ESI) [M+H]⁺: m/z 365.

### Intermediate 53: (5-(furan-2-yl)-1-(tetrahydro-2H-pyran-2-yl)-6-(trifluoromethyl)-1H-indazol-4-yl)boronic acid (Int-53)

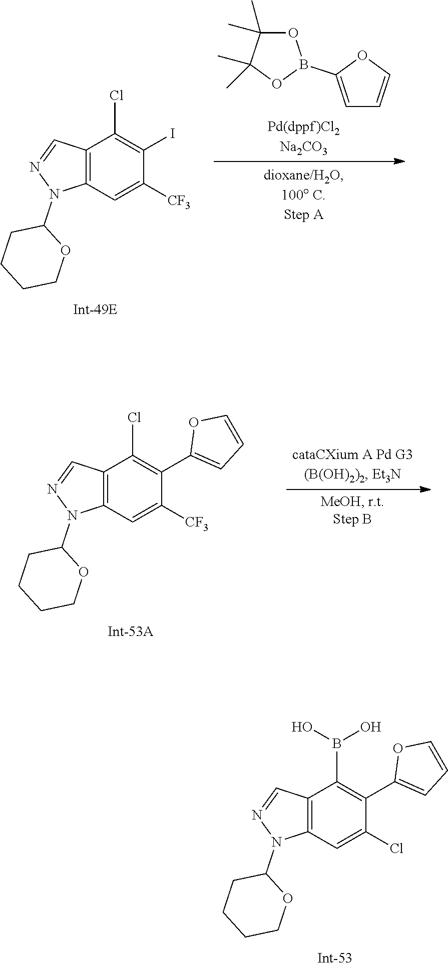 [SMILES: FC(F)(F)c1cc2c(cnn2C2CCCCO2)c(Cl)c1I ; CC1(C)OB(c2ccco2)OC1(C)C ; FC(F)(F)c1cc2c(cnn2C2CCCCO2)c(Cl)c1-c1ccco1 ; OB(O)c1c(-c2ccco2)c(Cl)cc2c1cnn2C1CCCCO1]

### Step A: 4-chloro-5-(furan-2-yl)-1-(tetrahydro-2H-pyran-2-yl)-6-(trifluoromethyl)-1H-indazole (Int-53A)

A mixture of 4-chloro-5-iodo-1-(tetrahydro-2H-pyran-2-yl)-6-(trifluoromethyl)-1H-indazole (Int-49E) (215 mg, 0.499 mmol), furan-2-boronic acid pinacol ester (0.2 mL, 1 mmol), [1,1′-bis(diphenylphosphino)ferrocene]dichloropalladium (II) (67 mg, 0.0916 mmol), Na₂CO₃ (2 M in H₂O) in 1,4-dioxane (2.5 mL) was stirred at 100° C. for 5 h. The resulting mixture was cooled to room temperature and diluted with EtOAc. The mixture was dried over Na₂SO₄, filtered, and concentrated. The residue was purified by silica gel chromatography (hexane-EtOAc) to give 4-chloro-5-(furan-2-yl)-1-(tetrahydro-2H-pyran-2-yl)-6-(trifluoromethyl)-/H-indazole (Int-53A). MS (ESI) [M+H]⁺: m/z 371.

### Step B: (5-(furan-2-yl)-1-(tetrahydro-2H-pyran-2-yl)-6-(trifluoromethyl)-1H-indazol-4-yl)boronic acid (Int-53)

Tetrahydroxydiboron (180 mg, 2.01 mmol), triethylamine (0.37 mL, 2.66 mmol), and cataCXium A Pd G3 (0.026 g, 0.0357 mmol) was added to a solution of 4-chloro-5-(furan-2-yl)-1-(tetrahydro-2H-pyran-2-yl)-6-(trifluoromethyl)-1H-indazole (Int-53A) (163 mg, 0.439 mmol) in MeOH (4 mL). The mixture was stirred at room temperature for 1.5 h under N₂. The reaction mixture was diluted with H₃PO₄ (10% aq.) and CHCl₃. The organic layer was separated, washed with brine, dried over Na₂SO₄, filtered, and concentrated. The residue was purified by flash silica gel chromatography (hexane-EtOAc/ethanol (4/1)) to afford (5-(furan-2-yl)-1-(tetrahydro-2H-pyran-2-yl)-6-(trifluoromethyl)-1H-indazol-4-yl)boronic acid (Int-53). MS (ESI) [M+H]⁺: m/z 381.

### Intermediate 54: (5-(cyclobut-1-en-1-yl)-1-(tetrahydro-2H-pyran-2-yl)-6-(trifluoromethyl)-1H-indazol-4-yl)boronic acid (Int-54)

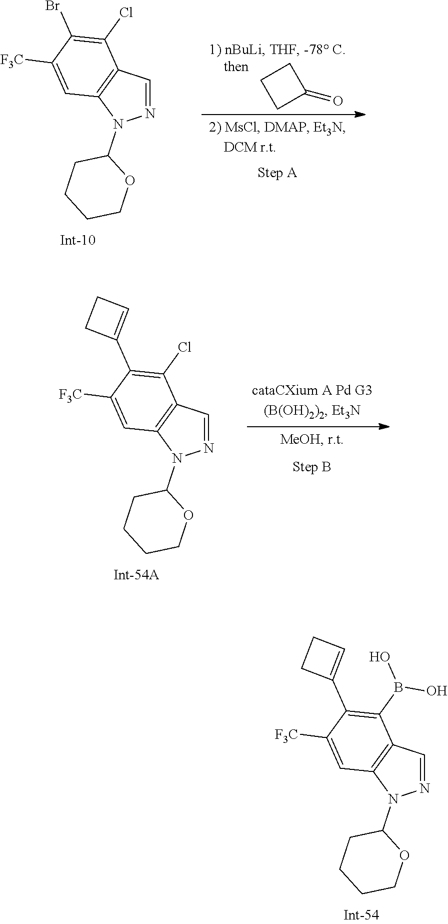 [SMILES: FC(F)(F)c1cc2c(cnn2C2CCCCO2)c(Cl)c1Br ; O=C1CCC1 ; FC(F)(F)c1cc2c(cnn2C2CCCCO2)c(Cl)c1C1=CCC1 ; OB(O)c1c(C2=CCC2)c(C(F)(F)F)cc2c1cnn2C1CCCCO1]

### Step A: 4-chloro-5-(cyclobut-1-en-1-yl)-1-(tetrahydro-2H-pyran-2-yl)-6-(trifluoromethyl)-1H-indazole (Int-54A)

n-butyllithium (1.60 mL, 2.35 mmol, 1.5 M in hexane) was added to a solution of 5-bromo-4-chloro-1-(tetrahydro-2H-pyran-2-yl)-6-(trifluoromethyl)-1H-indazole (Int-10) (600 mg, 1.56 mmol) in toluene (6.0 mL) and THF (1.5 mL) at −78° C. After stirring the mixture at −78° C. for 30 min, cyclobutanone (0.584 mL, 7.82 mmol) was added to the reaction mixture at −78° C. After stirring for 3 h, the reaction was quenched with sat. aq. NH₄Cl. The reaction mixture was extracted with EtOAc, and the combined organic layers were washed with brine, dried over Na₂SO₄, filtered and concentrated in vacuo. The residue was purified by flash silica gel chromatography (4 to 25% EtOAc in hexane).

Methanesulfonyl chloride (0.160 mL, 2.06 mmol) was added to a solution of the crude residue, triethylamine (0.285 mL, 2.05 mmol), and DMAP (209 mg, 1.71 mmol) in DCM (2.6 mL) at room temperature. After stirring the mixture at room temperature for 10 days, the reaction was quenched with H₂O. The reaction mixture was extracted with CHCl₃ and the combined organic layers were washed with brine, dried over Na₂SO₄, filtered, and concentrated in vacuo. The residue was purified by flash silica gel chromatography (17 to 38% EtOAc in hexane) to afford 4-chloro-5-(cyclobut-1-en-1-yl)-1-(tetrahydro-2H-pyran-2-yl)-6-(trifluoromethyl)-1H-indazole (Int-54A). ESI-MS m/z [M+H]⁺ 357.

### Step B: (5-(cyclobut-1-en-1-yl)-1-(tetrahydro-2H-pyran-2-yl)-6-(trifluoromethyl)-1H-indazol-4-yl)boronic acid (Int-54)

Triethylamine (0.107 mL, 0.768 mmol) was added to a solution of 4-chloro-5-(cyclobut-1-en-1-yl)-1-(tetrahydro-2H-pyran-2-yl)-6-(trifluoromethyl)-1H-indazole (Int-54A) (68.5 mg, 0.192 mmol), tetrahydroxydiboron (34.4 mg, 0.384 mmol), and cataCXium A Pd G3 (14.0 mg, 0.0192 mmol) in MeOH (3.8 mL) at room temperature. After stirring the mixture at room temperature for 2 h, the reaction was quenched with H₂O. The reaction mixture was extracted with CHCl₃ and the combined organic layers were washed with brine, dried over Na₂SO₄, filtered, and concentrated in vacuo. The residue was purified by flash silica gel chromatography (37 to 58% EtOAc in hexane) to afford (5-(cyclobut-1-en-1-yl)-1-(tetrahydro-2H-pyran-2-yl)-6-(trifluoromethyl)-1H-indazol-4-yl)boronic acid (Int-54). ESI-MS m/z [M+H]⁺ 367. ¹H-NMR. (400 MHz, CDCl₃) δ 8.21 (s, 1H), 7.99 (s, 1H), 6.26 (s, 1H), 5.97 (s, 2H), 5.75 (dd, J=9.3, 2.8 Hz, 1H), 4.08-4.00 (m, 1H), 3.82-3.73 (m, 1H), 2.93 (d, J=2.5 Hz, 2H), 2.62-2.47 (m, 3H), 2.21-2.04 (m, 2H), 1.84-1.63 (m, 3H).

### Intermediate 55: (6-cyclopropyl-5-(1-fluorocyclopropyl)-1-(tetrahydro-2H-pyran-2-yl)-1H-indazol-4-yl)boronic acid (Int-55)

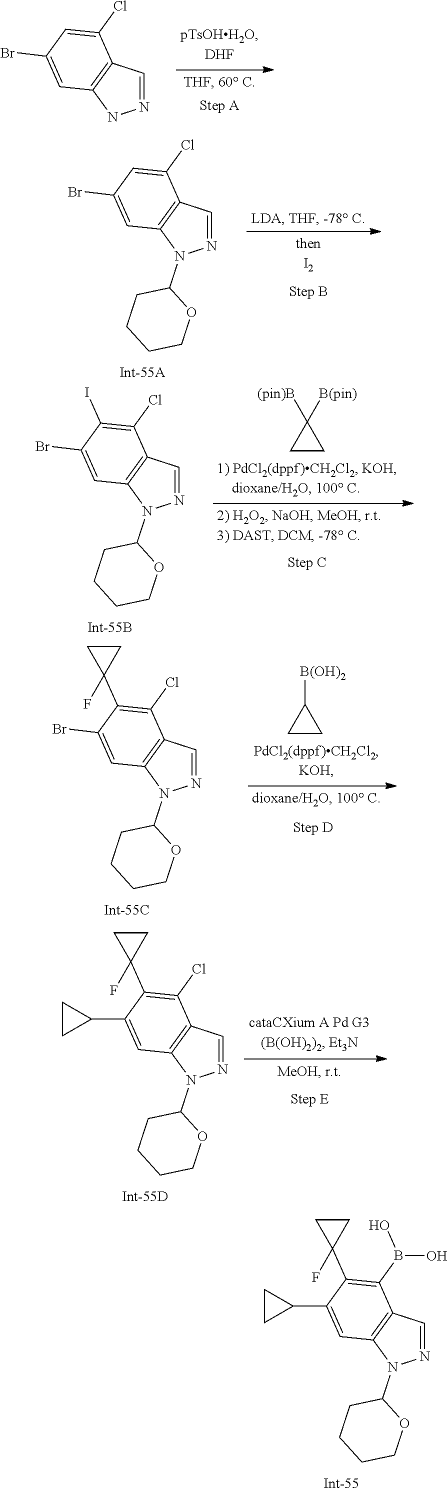

### Step A: 6-bromo-4-chloro-1-(tetrahydro-2H-pyran-2-yl)-1H-indazole (Int-55A)

3,4-dihydro-2H-pyran (5.91 mL, 64.8 mmol) was added to a solution of 6-bromo-4-chloro-1H-indazole (5.00 g, 21.6 mmol) and p-toluenesulfonic acid monohydrate (411 mg, 2.16 mmol) in THF (50 mL) at room temperature. The reaction was stirred at 60° C. for 2 h. cooled to room temperature, and quenched with triethylamine (0.602 mL, 4.32 mmol). The reaction mixture was concentrated in vacuo and purified by flash silica gel chromatography (0 to 40% EtOAc in hexane) to afford 6-bromo-4-chloro-1-(tetrahydro-2H-pyran-2-yl)-1H-indazole (Int-55A). ESI-MS m/z [M+H]⁺ 315, 317, 319. ¹H-NMR (400 MHz, CDCl₃) δ 8.05 (d, J=0.8 Hz, 1H), 7.71 (t, J=1.1 Hz, 1H), 7.30 (d, J=1.3 Hz, 1H), 5.66 (dd, J=9.1, 2.9 Hz, 1H), 4.04-3.96 (m, 1H), 3.79-3.70 (m, 1H), 2.55-2.42 (m, 1H), 2.20-2.03 (m, 2H), 1.84-1.64 (m, 3H).

### Step B: 6-bromo-4-chloro-5-iodo-1-(tetrahydro-2H-pyran-2-yl)-1H-indazole (Int-55B)

LDA (9.51 mL, 9.51 mmol, 1M in THF) was added to a solution of 6-bromo-4-chloro-1-(tetrahydro-2H-pyran-2-yl)-1H-indazole (Int-55A) (2.00 g, 6.34 mmol) in THF (100 mL) at −78° C. After stirring the mixture for 5 min, iodine (3.22 g, 12.7 mmol) in THF (20 mL) was added to the reaction mixture. After stirring the mixture at −78° C. for 1 h, the reaction was warmed up to room temperature and quenched with sat. aq. NH₄Cl. The reaction mixture was extracted with EtOAc and the organic phase was washed with brine, dried over Na₂SO₄, filtered and concentrated in vacuo. The crude residue was purified by flash silica gel chromatography (0 to 100% CHCl₃ in hexane) to afford 6-bromo-4-chloro-5-iodo-1-(tetrahydro-2H-pyran-2-yl)-1H-indazole (Int-55B). ESI-MS m/z [M+H]⁺ 441, 443, 445. ¹H-NMR (400 MHz, CDCl₃) δ 7.99 (d, J=1.0 Hz, 1H), 7.93 (d, J=1.0 Hz, 1H), 5.65 (dd, J=8.9, 2.9 Hz, 1H), 4.02-3.94 (m, 1H), 3.78-3.70 (m, 1H), 2.52-2.40 (m, 1H), 2.19-2.03 (m, 2H), 1.81-1.65 (m, 3H).

### Step C: 6-bromo-4-chloro-5-(1-fluorocyclopropyl)-1-(tetrahydro-2H-pyran-2-yl)-1H-indazole (Int-55C)

KOH (1.13 mL, 2.27 mmol, 2M in H₂O) was added to a solution of 6-bromo-4-chloro-5-iodo-1-(tetrahydro-2H-pyran-2-yl)-1H-indazole (Int-55B) (400 mg, 0.906 mmol), 4,4,5,5-tetramethyl-2-[1-(4,4,5,5-tetramethyl-1,3,2-dioxaborolan-2-yl)cyclopropyl]-1,3,2-dioxaborolane (666 mg, 2.27 mmol) and Pd(dppf)Cl₂·CH₂Cl (148 mg, 0.181 mmol) in dioxane (3.6 mL) at room temperature. After stirring the mixture at 100° C. for 13 h, the reaction was cooled to room temperature and quenched with H₂O. The reaction mixture was extracted with EtOAc and the organic phase was washed with brine, dried over Na₂SO₄, filtered and concentrated in vacuo. The crude residue was purified by flash silica gel chromatography (10 to 100% EtOAc in hexane) to afford 6-bromo-4-chloro-1-(tetrahydro-2H-pyran-2-yl)-5-(1-(4,4,5,5-tetramethyl-1,3,2-dioxaborolan-2-yl)cyclopropyl)-1H-indazole. ESI-MS m/z [M+H]⁺ 481, 483, 485.

NaOH (3.64 mL, 7.25 mmol, 2 M in H₂O) and H₂O₂ (1.02 mL, 9.06 mmol, 30% w/w) were added to a solution of 6-bromo-4-chloro-1-(tetrahydro-2H-pyran-2-yl)-5-(1-(4,4,5,5-tetramethyl-1,3,2-dioxaborolan-2-yl)cyclopropyl)-1H-indazole in THF (9.1 mL) and MeOH (0.91 mL) at 0° C. After warming the mixture to room temperature and stirring for 30 min, the reaction was quenched with sat. aq. NaHCO₃ and sat. aq. Na₂S₂O₃. The reaction mixture was extracted with EtOAc, washed with brine, dried over Na₂SO₄, filtered, and concentrated in vacuo. The residue was purified by flash silica gel chromatography (0 to 30% EtOAc in hexane) to afford 1-(6-bromo-4-chloro-1-(tetrahydro-2H-pyran-2-yl)-1H-indazol-5-yl)cyclopropan-1-ol. ESI-MS m/z [M+H]⁺ 371, 373, 375.

Diethylaminosulfur trifluoride (0.117 mL, 0.883 mmol) was added to a solution of 1-(6-bromo-4-chloro-1-(tetrahydro-2H-pyran-2-yl)-1H-indazol-5-yl)cyclopropan-1-ol (164 mg, 0.441 mmol) in dichloromethane (8.8 mL) at −78° C. After stirring the mixture at −78° C. for 30 min, the reaction was quenched with sat. aq. NaHCO₃. The reaction mixture was extracted with CHCl₃, washed with brine, dried over Na₂SO₄, filtered, and concentrated in vacuo. The residue was purified by flash silica gel chromatography (0 to 30% EtOAc in hexane) to afford 6-bromo-4-chloro-5-(1-fluorocyclopropyl)-1-(tetrahydro-2H-pyran-2-yl)-1H-indazole (Int-55C). ESI-MS m/z [M+H]⁺ 373, 375, 379. ¹H-NMR (400 MHz, CDCl₃) δ 8.11 (d, J=0.5 Hz, 1H), 7.87 (t, J=0.8 Hz, 1H), 5.66 (dd, J=9.0, 2.8 Hz, 1H), 4.03-3.95 (m, 1H), 3.79-3.70 (m, 1H), 2.53-2.41 (m, 1H), 2.19-2.04 (m, 2H), 1.83-1.63 (m, 5H), 1.24-1.15 (m, 2H).

### Step D: 4-chloro-6-cyclopropyl-5-(1-fluorocyclopropyl)-1-(tetrahydro-2H-pyran-2-yl)-1H-indazole (Int-55D)

KOH (0.254 mL, 0.254 mmol, 1M in H₂O) was added to a solution of 6-bromo-4-chloro-5-(1-fluorocyclopropyl)-1-(tetrahydro-2H-pyran-2-yl)-1H-indazole (Int-55C) (19.0 mg, 0.0509 mmol), cyclopropylboronic acid (8.7 mg, 0.102 mmol), and Pd(dppf)Cl₂′ CH₂Cl₂ (8.3 mg, 0.0102 mmol) in dioxane (1.0 mL) at room temperature. After stirring the mixture at 100° C. for 2 h, the reaction was cooled to room temperature and quenched with H₂O. The reaction mixture was extracted with EtOAc, washed with brine, dried over Na₂SO₄, filtered, and concentrated in vacuo. The residue was purified by flash silica gel chromatography (0 to 20% EtOAc in hexane) to afford 4-chloro-6-cyclopropyl-5-(1-fluorocyclopropyl)-1-(tetrahydro-2H-pyran-2-yl)-1H-indazole (Int-55D). ESI-MS m/z [M+H]⁺ 335, 337.

### Step E: (6-cyclopropyl-S-(1-fluorocyclopropyl)-1-(tetrahydro-2H-pyran-2-yl)-1H-indazol-4-yl)boronic acid (Int-55)

Triethylamine (0.0133 mL, 0.0956 mmol) was added to a solution of 4-chloro-6-cyclopropyl-5-(1-fluorocyclopropyl)-1-(tetrahydro-2H-pyran-2-yl)-1H-indazole (Int-55D) (8.0 mg, 0.0234 mmol), tetrahydroxydiboron (4.3 mg, 0.0478 mmol), and cataCXium A Pd G3 (1.7 mg, 0.00239 mmol) in MeOH (0.48 mL) at room temperature. After stirring the mixture at room temperature for 15 h, the reaction was quenched with H₂O. The reaction mixture was extracted with CHCl₃, washed with brine, dried over Na₂SO₄, filtered, and concentrated in vacuo. The residue was purified by flash silica gel chromatography (10 to 50% EtOAc in hexane) to afford (6-cyclopropyl-5-(1-fluorocyclopropyl)-1-(tetrahydro-2H-pyran-2-yl)-1H-indazol-4-yl)boronic acid (Int-55). ESI-MS m/z [M+H]⁺ 345. ¹H-NMR (400 MHz, CDCl₃) δ 8.19 (s, 1H), 7.20 (s, 1H), 5.68 (dd, J=9.1, 2.6 Hz, 1H), 5.19 (d. J=2.0 Hz, 2H), 4.04-3.95 (m, 1H), 3.79-3.69 (m, 1H), 2.61-2.47 (m, 1H), 2.20-2.11 (m, 1H), 2.08-1.96 (m, 2H), 1.81-1.63 (m, 3H), 1.18-1.05 (m, 4H), 0.93-0.80 (m, 4H).

### Intermediate 56: (1-(4-methoxybenzyl)-6-methyl-5-(trifluoromethyl)-1H-pyrazolo[3,4-b]pyridin-4-yl)boronic acid (Int-56)

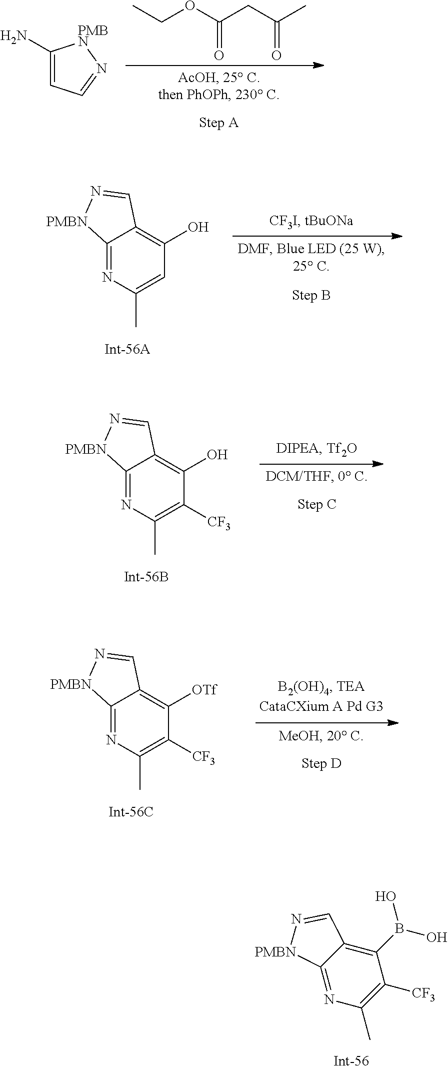

### Step A: 1-(4-methoxybenzyl)-6-methyl-1H-pyrazolo[3,4-b]pyridin-4-ol (Int-56A)

To a solution of 1-(4-methoxybenzyl)-1H-pyrazol-5-amine (35 g, 170 mmol) in AcOH (400 mL) was added ethyl acetoacetate (100 g, 770 mmol) at 25° C., and the reaction was stirred at 25° C. for 16 h. The reaction mixture was concentrated in vacuo and the residue was dissolved in diphenyl ether (350 mL). The resulting mixture was stirred at 230° C. for 90 min. After cooling to r.t., the reaction mixture was poured into petroleum ether (300 mL). The resulting mixture was filtered and the solid was collected to give 1-(4-methoxybenzyl)-6-methyl-1H-pyrazolo[3,4-b]pyridin-4-ol (Int-56A). MS (ESI) [M+H]⁺: m/z 270. ¹H NMR (400 MHz, DMSO) δ 11.37 (br s, 1H), 8.07-7.78 (m, 1H), 7.30-7.07 (m, 2H), 6.86 (br d, J=8.3 Hz, 2H), 6.40 (s, 1H), 5.55-5.28 (m, 2H), 3.70 (s, 3H), 2.50-2.43 (m, 3H).

### Step B: 1-(4-methoxybenzyl)-6-methyl-5-(trifluoromethyl)-1H-pyrazolo[3,4-b]pyridin-4-ol (Int-56B)

To a solution of 1-(4-methoxybenzyl)-6-methyl-1H-pyrazolo[3,4-b]pyridin-4-ol (Int-56A) (13 g, 48 mmol) in DMF (480 mL) was added sodium tert-butoxide (9.3 g, 97 mmol) and trifluoroiodomethane (64 g, 82 mmol, 25% w/w in DMF) at 25° C., and the reaction was stirred at 25° C. under blue LED (25 W) irradiation for 12 b. The reaction mixture was concentrated in vacuo and the residue was diluted with EtOAc (300 mL). The resulting mixture adjusted to pH 7 by the addition of IM aq. HCl. The mixture was extracted with EtOAc (2×300 mL), and the combined organic phase was washed with brine (3×100 mL). The combined organic phase was dried over anhydrous Na₂SO₄, filtered, and concentrated in vacuo. The residue was purified by flash silica gel chromatography (petroleum ether/EtOAc/EtOH=8/3/1) to give 1-(4-methoxybenzyl)-6-methyl-5-(trifluoromethyl)-1H-pyrazolo[3,4-b]pyridin-4-ol (Int-56B). MS (ESI) [M+H]⁺: m/z 338. ¹H NMR (400 MHz, DMSO-d₆) δ 12.83-12.08 (m, 1H), 8.38-7.82 (m, 1H), 7.18 (d, J=8.6 Hz, 2H), 7.00-6.75 (m, 2H), 5.54-5.35 (m, 2H), 3.78-3.61 (m, 3H), 2.70-2.53 (m, 3H).

### Step C: 1-(4-methoxybenzyl)-6-methyl-5-(trifluoromethyl)-1H-pyrazolo[3,4-b]pyridin-4-yl trifluoromethanesulfonate (Int-56C)

To a solution of 1-(4-methoxybenzyl)-6-methyl-5-(trifluoromethyl)-1H-pyrazolo[3,4-b]pyridin-4-ol (Int-56B) (10.7 g, 31.7 mmol) and DIPEA (33.2 mL, 190 mmol) in DCM (100 mL) and THF (30 mL) was added Tf₂O (13.4 mL, 79.0 mmol) at 0° C., and the reaction was stirred at 0° C. for 10 min. The reaction mixture was concentrated in vacuo, and the residue was purified by flash silica gel chromatography (petroleum ether/EtOAc=5/1) to give 1-(4-methoxybenzyl)-6-methyl-5-(trifluoromethyl)-1H-pyrazolo[3,4-b]pyridin-4-yl trifluoromethanesulfonate (Int-56C). MS (ESI) [M+H]⁺: m/z 470. ¹H NMR (400 MHz, CDCl₃) δ 8.08 (s, 1H), 7.33-7.24 (m, 2H), 6.84-6.72 (m, 2H), 5.55 (s, 2H), 3.70 (s, 3H), 2.83 (q, J=2.4 Hz, 3H).

### Step D: (1-(4-methoxybenzyl)-6-methyl-5-(trifluoromethyl)-1H-pyrazolo[3,4-b]pyridin-4-yl)boronic acid (Int-56)

To a solution of 1-(4-methoxybenzyl)-6-methyl-5-(trifluoromethyl)-1H-pyrazolo[3,4-b]pyridin-4-yl trifluoromethanesulfonate (Int-56C) (500 mg, 1.07 mmol) in MeOH (5.00 mL) was added tetrahydroxydiboron (382 mg, 4.26 mmol), CataCXium A Pd G3 (43 mg, 0.053 mmol), and TEA (0.445 mL, 3.20 mmol) at 20° C. under N₂ atmosphere. The reaction mixture was stirred at 20° C. for 2 h. The reaction solution was evaporated under reduced pressure to give the crude product. The crude product was purified by flash silica gel chromatography (0-100% EtOAc in petroleum ether) to give (1-(4-methoxybenzyl)-6-methyl-5-(trifluoromethyl)-1H-pyrazolo[3,4-b]pyridin-4-yl)boronic acid (Int-56). MS (ESI) [M+H]⁺: m/z 366.

### Intermediate 57: (6-chloro-1-(4-methoxybenzyl)-5-(trifluoromethyl)-1H-indazol-4-yl)boronic acid (Int-57)

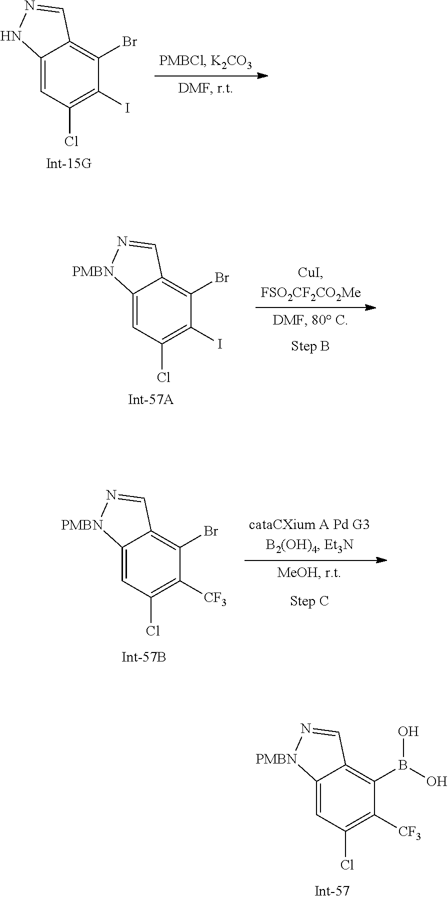

### Step A: 4-bromo-6-chloro-5-iodo-1-(4-methoxybenzyl)-1H-indazole (Int-57A)

To a solution of 4-bromo-6-chloro-5-iodo-1H-indazole (Int-15G) (2 g, 5.60 mmol) in DMF (20 ml) was added K₂CO₃ (2.320 g, 16.79 mmol) and 1-(chloromethyl)-4-methoxybenzene (1.753 g, 11.19 mmol) at 25° C. The mixture was stirred at r.t. for 12 h. The mixture was diluted with water (20 mL) and extracted with EtOAc (3×50 mL). The organic layer was washed with brine (2×15 mL), dried over sodium sulfate, filtered and the solvent was evaporated under reduced pressure to give a crude product. The crude product was purified by flash silica gel chromatography (0 to 30% ethyl acetate in petroleum ether) to give 4-bromo-6-chloro-5-iodo-1-(4-methoxybenzyl)-1H-indazole (Int-57A). MS (ESI) [M+H]⁺: m/z 477.

### Step B: 4-bromo-6-chloro-1-(4-methoxybenzyl)-5-(trifluoromethyl)-1H-indazole (Int-57B)

To a solution of 4-bromo-6-chloro-5-iodo-1-(4-methoxybenzyl)-1H-indazole (Int-57A) (100 mg, 0.209 mmol) in DMF (2 ml) was added copper (I) iodide (120 mg, 0.628 mmol) and methyl 2,2-difluoro-2-(fluorosulfonyl)acetate (161 mg, 0.838 mmol) at 25° C. under N₂ atmosphere. The mixture was stirred at 80° C. for 5.5 h. The mixture was cooled, quenched with conc. aq. NH₃ (1 mL), diluted with water and ethyl acetate (4 mL each), and filtered. The filtrate was extracted with ethyl acetate (3×4 mL), washed with brine (3 mL), dried over Na₂SO₄, filtered, and the solvent was evaporated under reduced pressure to give the crude product. The residue was purified by preparative TLC (SiO₂, petroleum ether:ethyl acetate=5:1) to give 4-bromo-6-chloro-1-(4-methoxybenzyl)-5-(trifluoromethyl)-1H-indazole (Int-57B). [M+H]⁺: m/z 419.

### Step C: (6-chloro-1-(4-methoxybenzyl)-5-(trifluoromethyl)-1H-indazol-4-ylboronic acid (Int-57)

To a solution of 4-bromo-6-chloro-1-(4-methoxybenzyl)-5-(trifluoromethyl)-1H-indazole (Int-57B) (200 mg, 0.477 mmol) in MeOH (3 ml) was added tetrahydroxydiboron (171 mg, 1.906 mmol), Et₃N (0.199 ml, 1.430 mmol), and cataCXium A Pd G3 (15.93 mg, 0.024 mmol), and the reaction mixture was stirred at r.t. for 48 h. The mixture was diluted with water (3 mL) and HCl (0.5M, 0.5 mL) and extracted with EtOAc (3×5 mL). The organic layer was washed with brine (3×10 mL), dried over sodium sulfate, filtered, and the solvent was evaporated under reduced pressure to give a crude product. The crude product was purified by reverse-phase MPLC (0 to 15% MeCN in H₂O (0.05% TFA)) to give (6-chloro-1-(4-methoxybenzyl)-5-(trifluoromethyl)-1H-indazol-4-yl)boronic acid (Int-20). [M+H]⁺: m/z 385.

### Intermediate 58: 3-(4,4,5,5-tetramethyl-1,3,2-dioxaborolan-2-yl)-4-(triisopropylsilyl)ethynyl)-1-((2-(trimethylsilyl)ethoxy)methyl)-1H-indazole (Int-58)

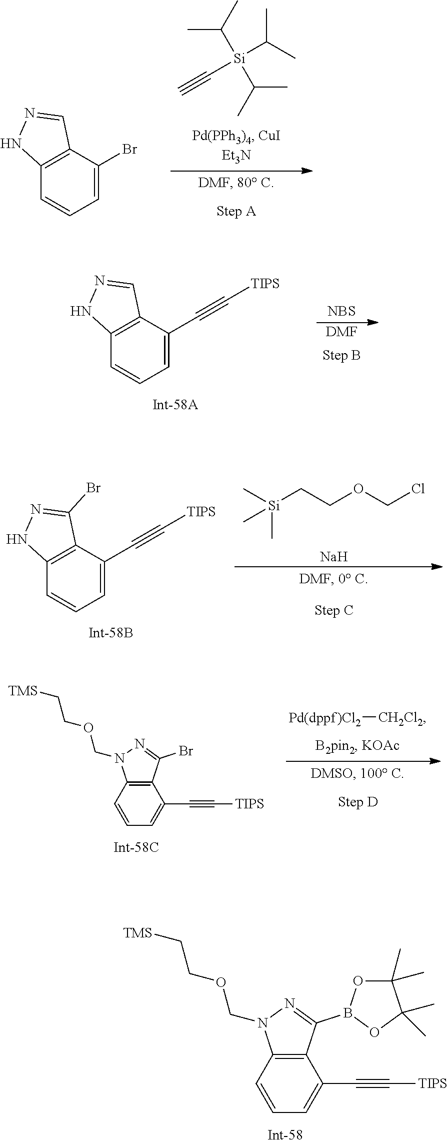

### Step A: 4-((triisopropylsilyl)ethynyl)-1H-indazole (Int-58A)

A mixture of 4-bromo-1H-indazole (1.27 g, 6.45 mmol), ethynyl(triisopropyl) silane (1.76 g, 9.67 mmol), copper (I) iodide (0.246 g, 1.29 mmol), triethylamine (4.49 ml, 32.2 mmol), and tetrakis(triphenylphosphine) palladium (0) (0.745 g, 0.645 mmol) in DMF (10 mL) was degassed, charged with N₂, then stirred at 80° C. for 16 h. After cooling, the mixture was partitioned between EtOAc and water. The organic phase was washed with water and brine, dried over sodium sulfate, filtered and concentrated. The crude residue was purified via silica gel chromatography (5 to 30% EtOAc in hexane) to give 4-((triisopropylsilyl)ethynyl)-1H-indazole (Int-58A). MS (ESI) [M+H]⁺: m/z 300, ¹H NMR (400 MHz, CDCl₃) δ=10.52-9.79 (m, 1H), 8.24 (br s, 1H), 7.52-7.44 (m, 1H), 7.38-7.34 (m, 2H), 1.22-1.18 (m, 21H).

### Step B: 3-bromo-4-((triisopropylsilyl)ethynyl)-1H-indazole (Int-58B)

To a stirred solution of 4-((triisopropylsilyl)ethynyl)-1H-indazole (Int-58A) (1.34 g, 4.49 mmol) in DMF (12 ml) was added N-bromosuccinimide (0.879 g, 4.94 mmol) and stirred at room temperature for 2 h. Then the mixture was partitioned between EtOAc and water. The organic phase was washed with water and brine, dried over sodium sulfate, filtered and concentrated to give 3-bromo-4-((triisopropylsilyl)ethynyl)-1H-indazole (Int-58B) which was used next step without further purification. MS (ESI) [M+H]⁺: m/z 378.

### Step C: 3-bromo-4-((triisopropylsilyl)ethynyl)-1-((2-(trimethylsilyl)ethoxy)methyl)-1H-indazole (Int-58C)

To a stirred mixture of 3-bromo-4-((triisopropylsilyl)ethynyl)-1H-indazole (Int-58B) (500 mg, 1.325 mmol) in DMF (4.4 mL) was added sodium hydride (79 mg, 1.99 mmol, 60 wt %) at 0° C. After 5 min, (2-(chloromethoxy)ethyl)trimethylsilane (0.37 ml, 2.12 mmol) was added to the mixture and allowed to stir for another 1 h at 0° C. Then the mixture was partitioned between water and EtOAc. The organic phase was washed with water and brine, dried over sodium sulfate, filtered and concentrated to give 3-bromo-4-((triisopropylsilyl)ethynyl)-1-((2-(trimethylsilyl)ethoxy)methyl)-1H-indazole (Int-58C) which was used directly in the next step without further purification. MS (ESI) [M+H]⁺: m/z 507.

### Step D: 3-(4,4,5,5-tetramethyl-1,3,2-dioxaborolan-2-yl)-4-((triisopropylsilyl)ethynyl)-1-((2-(trimethylsilyl)ethoxy)methyl)-1H-indazole (Int-58)

A mixture of 3-bromo-4-((triisopropylsilyl)ethynyl)-1-((2-(trimethylsilyl)ethoxy)methyl)-1H-indazole (Int-58C) (231 mg, 0.421 mmol), bis(pinacolato)diboron (214 mg, 0.842 mmol), potassium acetate (165 mg, 1.68 mmol), [1,1′-bis(diphenylphosphino)ferrocene]palladium (II) dichloride dichloromethane complex (34 mg, 0.042 mmol) in DMSO (4.2 ml) was degassed and charged with nitrogen (3 times) and stirred at 100° C. for 5 h. Then another portion of bis(pinacolato)diboron (214 mg, 0.842 mmol) and [1,1′-bis(diphenylphosphino)ferrocene]palladium (II) dichloride dichloromethane complex (34 mg, 0.042 mmol) was added to the mixture and stirred at 100° C. for another 11 h. Then the mixture was partitioned between water and EtOAc. The organic phase was washed with water and brine, dried over sodium sulfate, filtered and concentrated. The crude residue was purified via silica gel chromatography (5 to 100% acetone in hexane) to give desired 3-(4,4,5,5-tetramethyl-1,3,2-dioxaborolan-2-yl)-4-((triisopropylsilyl)ethynyl)-1-((2-(trimethylsilyl)ethoxy)methyl)-1H-indazole (Int-58). MS (ESI) [M+H]⁺: m/z 473 (corresponding boronic acid was observed). ¹H NMR (400 MHz, CDCl₃) δ=7.68-7.59 (m, 1H), 7.56-7.49 (m, 1H), 7.37-7.29 (m, 1H), 5.94-5.71 (m, 2H), 3.73-3.56 (m, 2H), 1.44-1.43 (m, 3H), 1.30-1.28 (m, 18H), 1.27 (s, 12H), 0.99-0.84 (m, 2H), −0.05 (s, 9H).

### Intermediate 59: (6-fluoro-1-((trifluoromethyl) sulfonyl)-5-((triisopropylsilyl)ethynyl)-1H-benzo[f]indazol-4-yl)boronic acid (Int-59)

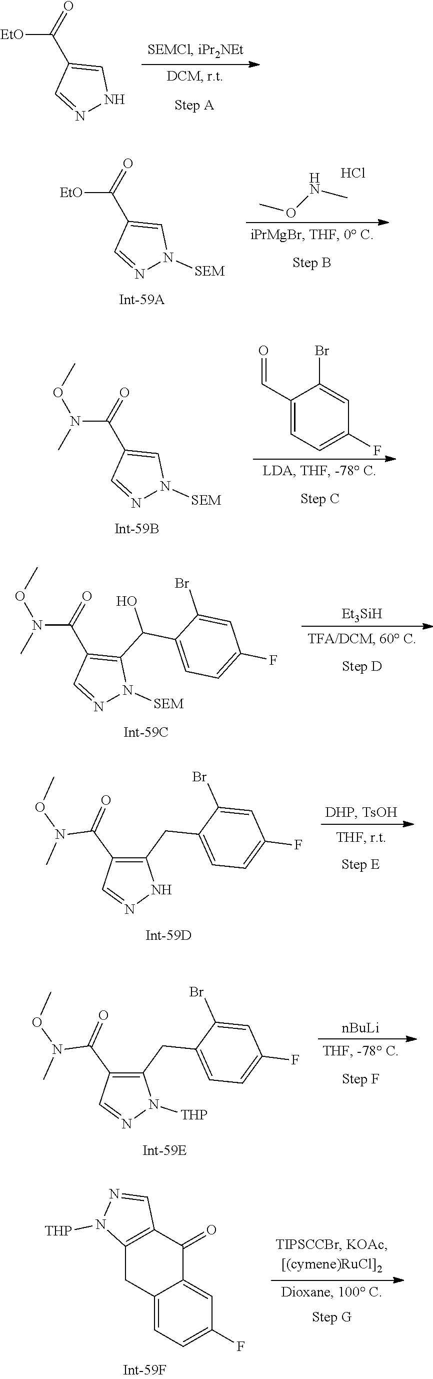

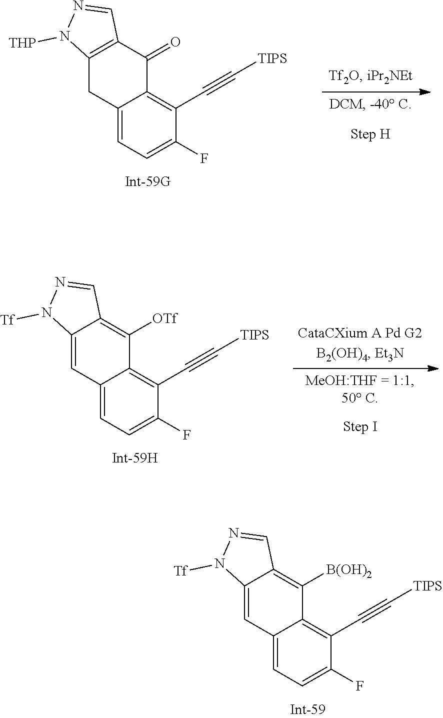

### Step A: Ethyl 1-((2-(trimethylsilyl)ethoxy)methyl)-1H-pyrazole-4-carboxylate (Int-59A)

To a solution of ethyl 1H-pyrazole-4-carboxylate (13.8 g, 98.0 mmol) in DCM (100 mL) was added N,N-diisopropylethylamine (25.4 g, 196 mmol), and the mixture was stirred for 15 min under 0° C., then (2-(chloromethoxy)ethyl)trimethylsilane (24.5 g, 147 mmol) was added slowly. The mixture was stirred at 25° C. for 16 h. The mixture was quenched with water (50 mL) and extracted with DCM (3×200 mL). The combined organic layer was dried over anhydrous sodium sulfate, filtered, and the filtrate was evaporated under reduced pressure. The crude product was purified by flash silica gel chromatography (0 to 35% EtOAc in petroleum ether gradient) to give ethyl 1-((2-(trimethylsilyl)ethoxy)methyl)-1H-pyrazole-4-carboxylate (Int-59A). MS (ESI) [M+H]⁺: m/z 271.

### Step B: N-methoxy-N-methyl-1-((2-(trimethylsilyl)ethoxy)methyl)-1H-pyrazole-4-carboxamide (Int-59B)

To a mixture of ethyl 1-((2-(trimethylsilyl)ethoxy)methyl)-1H-pyrazole-4-carboxylate (Int-59A) (20 g, 74 mmol) and N,O-dimethylhydroxylamine hydrochloride (10.8 g, 111 mmol) in dry THF (350 mL) was added iPrMgBr (77 mL, 222 mmol, 2.9 M in 2-methyltetrahydrofuran) at 0° C. The mixture was stirred at 25° C. for 16 h. The mixture was quenched with sat. aq. NH₄Cl (100 mL) and extracted with EtOAc (3×300 mL). The organic layer was dried over sodium sulfate, filtered, and evaporated under reduced pressure to give the crude product. The crude product was purified by flash silica gel chromatography (0 to 50% EtOAc in petroleum ether) to give N-methoxy-N-methyl-1-((2-(trimethylsilyl)ethoxy)methyl)-1H-pyrazole-4-carboxamide (Int-59B). MS (ESI) [M+H]⁺: m/z 286.

### Step C: 5-((2-bromo-4-fluorophenyl) (hydroxy)methyl)-N-methoxy-N-methyl-1-(2-(trimethylsilyl)ethoxy)methyl)-1H-pyrazole-4-carboxamide (Int-59C)

To a stirred solution of N-methoxy-N-methyl-1-((2-(trimethylsilyl)ethoxy)methyl)-1H-pyrazole-4-carboxamide (Int-59B) (24.7 g, 87.0 mmol) in THF (50 mL) was added lithium diisopropylamide (95 mL, 95 mmol, 1 M in THF) at −78° C. under N₂, and the reaction was stirred at −78° C. for 1 h. Then 2-bromo-4-fluorobenzaldehyde (16 g, 79 mmol) was added at −78° C. and the reaction was stirred at −78° C. for 1 h. The mixture was warmed to room temperature and concentrated in vacuum. The mixture was quenched with water (50 mL) and extracted with EtOAc (3×500 mL). The organic layers were washed with sat. brine (50 mL), dried over sodium sulfate, filtered, and concentrated in vacuum. The residue was purified by flash silica gel chromatography (0 to 25% EtOAc in petroleum ether) to give 5-((2-bromo-4-fluorophenyl) (hydroxy)methyl)-N-methoxy-N-methyl-1-((2-(trimethylsilyl)ethoxy)methyl)-1H-pyrazole-4-carboxamide (Int-59C). MS (ESI) [M+H]⁺: m/z 488, 490.

### Step D: 5-(2-bromo-4-fluorobenzyl)-N-methoxy-N-methyl-1H-pyrazole-4-carboxamide (Int-59D)

To a solution of 5-((2-bromo-4-fluorophenyl) (hydroxy)methyl)-N-methoxy-N-methyl-1-((2-(trimethylsilyl)ethoxy)methyl)-1H-pyrazole-4-carboxamide (Int-59C) (20.0 g, 40.9 mmol) in DCM (56 mL) was added triethylsilane (118 mL, 737 mmol) and TFA (56.8 mL, 737 mmol), and the mixture was stirred at 60° C. for 3 h. The reaction mixture was concentrated in vacuo and dissolved in EtOAc (400 mL). The reaction mixture was basified with sat. aq. NaHCO₃ to pH˜8. The organic layer was washed with brine (2×30 mL), dried with sodium sulfate, filtered, and concentrated in vacuo. The residue was purified by flash silica gel chromatography (0 to 15% ethyl acetate in petroleum ether) to give 5-(2-bromo-4-fluorobenzyl)-N-methoxy-N-methyl-1H-pyrazole-4-carboxamide (Int-59D). MS (ESI) [M+H]⁺: m/z 342, 344.

### Step E: 5-(2-bromo-4-fluorobenzyl)-N-methoxy-N-methyl-1-(tetrahydro-2H-pyran-2-yl)-1H-pyrazole-4-carboxamide (Int-59E)

To a solution of 5-(2-bromo-4-fluorobenzyl)-N-methoxy-N-methyl-1H-pyrazole-4-carboxamide (Int-59D) (7.50 g, 21.9 mmol) in THF (80 mL) was added 4-methylbenzenesulfonic acid (0.377 g, 2.19 mmol) and DHP (4.01 mL, 43.8 mmol) at 25° C., and the mixture was stirred for 2 h. The mixture was concentrated in vacuo and the residue was purified by flash silica gel chromatography (petroleum ether/EtOAc=2/1) to give 5-(2-bromo-4-fluorobenzyl)-N-methoxy-N-methyl-1-(tetrahydro-2H-pyran-2-yl)-1H-pyrazole-4-carboxamide (Int-59E). MS (ESI) [M+H]⁺: m/z 426, 428.

### Step F: 6-fluoro-1-(tetrahydro-2H-pyran-2-yl)-1.3a-dihydro-4H-benzo[f]indazol-4-one (Int-59F)

To a stirred solution of 5-(2-bromo-4-fluorobenzyl)-N-methoxy-N-methyl-1-(tetrahydro-2H-pyran-2-yl)-1H-pyrazole-4-carboxamide (Int-59E) (22 g, 51.6 mmol) in THF (220 mL) was added n-BuLi (31 mL, 77 mmol, 2.5 M in hexane) dropwise over 30 min at −78° C. under N₂. After the addition was finished, the reaction was stirred at −78° C. for 0.5 h. The reaction mixture was poured into sat. aq. NH₄Cl (200 mL) and diluted with EtOAc (200 mL). The organic layer was separated and the aqueous layer was extracted with EtOAc (3×200 mL). The combined organic layers were washed with brine (200 mL), dried over sodium sulfate, filtered, and concentrated in vacuo. The crude material was purified by flash silica gel chromatography (0 to 22% THF in petroleum ether) to give 6-fluoro-1-(tetrahydro-2H-pyran-2-yl)-1,3a-dihydro-4H-benzo[f]indazol-4-one (Int-59F). MS (ESI) [M+H]⁺: m/z 287.

### Step G: 6-fluoro-1-(tetrahydro-2H-pyran-2-yl)-5-(triisopropylsilyl)ethynyl)-1,9-dihydro-4H-benzo[f]indazol-4-one (Int-59G)

To a solution of 6-fluoro-1-(tetrahydro-2H-pyran-2-yl)-1,3a-dihydro-4H-benzo[f]indazol-4-one (Int-59F) (6.50 g, 22.7 mmol) in dioxane (120 mL) was added (bromoethynyl)triisopropylsilane (17.8 g, 68.1 mmol), potassium acetate (7.8 g, 79 mmol), and dichloro(p-cymene) ruthenium (II) dimer (6.95 g, 11.4 mmol) at 25° C. under N₂. The mixture was stirred at 100° C. for 3 h. The mixture was cooled, filtered, and the filtrate was concentrated in vacuo. The residue was purified by flash silica gel chromatography (2% ethyl acetate in DCM) to give 6-fluoro-1-(tetrahydro-2H-pyran-2-yl)-5-((triisopropylsilyl)ethynyl)-1,9-dihydro-4H-benzo[f]indazol-4-one (Int-59G). MS (ESI) [M+H]⁺: m/z 467.

### Step H: 6-fluoro-1-((trifluoromethyl) sulfonyl)-5-((triisopropylsilyl)ethynyl)-1H-benzo[f]indazol-4-yl trifluoromethanesulfonate (Int-59H)

A solution of 6-fluoro-1-(tetrahydro-2H-pyran-2-yl)-5-((triisopropylsilyl)ethynyl)-1,9-dihydro-4H-benzo[f]indazol-4-one (Int-59G) (5.5 g, 11.79 mmol) and N,N-diisopropylethylamine (12.4 mL, 70.7 mmol) in DCM (60 mL) was added Tf₂O (5.97 mL, 35.4 mmol) at −40° C., and the reaction mixture was stirred for 15 min. The reaction mixture was concentrated in vacuo at 30° C. and the residue was purified by flash silica gel chromatography (petroleum ether/EtOAc=20/1) to give 6-fluoro-1-((trifluoromethyl) sulfonyl)-5-((triisopropylsilyl)ethynyl)-1H-benzo[f]indazol-4-yl trifluoromethanesulfonate (Int-59H). ¹H NMR (400 MHz, CDCl₃) δ 8.71 (s, 1H), 8.50 (s, 1H), 8.04 (dd, J=5.36, 9.18 Hz, 1H), 7.55 (t, J=8.64 Hz, 1H), 1.17-1.24 (m, 21H).

### Step 1: (6-fluoro-1-((trifluoromethyl) sulfonyl)-5-((triisopropylsilyl)ethynyl)-1H-benzo[f]indazol-4-yl)boronic acid (Int-59)

To a solution of 6-fluoro-1-((trifluoromethyl) sulfonyl)-5-((triisopropylsilyl)ethynyl)-1H-benzo[f]indazol-4-yl trifluoromethanesulfonate (Int-59H) (3.20 g, 4.95 mmol), hypodiboric acid (3.11 g, 34.6 mmol), and CataCXium A Pd G2 (0.265 g, 0.396 mmol) in MeOH (40 mL) and THF (40 mL) was added triethylamine (2.07 mL, 14.9 mmol) at 25° C. and the mixture was stirred at 50° C. for 1 h under N₂. The reaction mixture was concentrated in vacuo and purified by flash silica gel chromatography (0 to 10% EtOAc in petroleum ether) to give (6-fluoro-1-((trifluoromethyl) sulfonyl)-5-((triisopropylsilyl)ethynyl)-1H-benzo[f]indazol-4-yl)boronic acid (Int-59). MS (ESI) [M+H]⁺; m/z 543.

The compound in the table below was synthesized using a similar procedure as described in the synthesis of Int-59 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-60 | 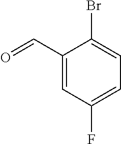 | 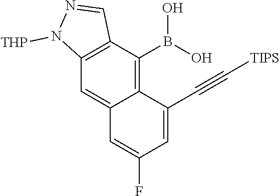 | (7-fluoro-1- (tetrahydro-2H- pyran-2-yl)-5- ((triisopropylsilyl) ethynyl)-1H- benzo[f]indazol-4- yl)boronic acid | 495 |

### Intermediate 61:5-chloro-4-(4,4,5,5-tetramethyl-1,3,2-dioxaborolan-2-yl)-1-((trifluoromethyl) sulfonyl)-1H-benzo[f]indazole (Int-61)

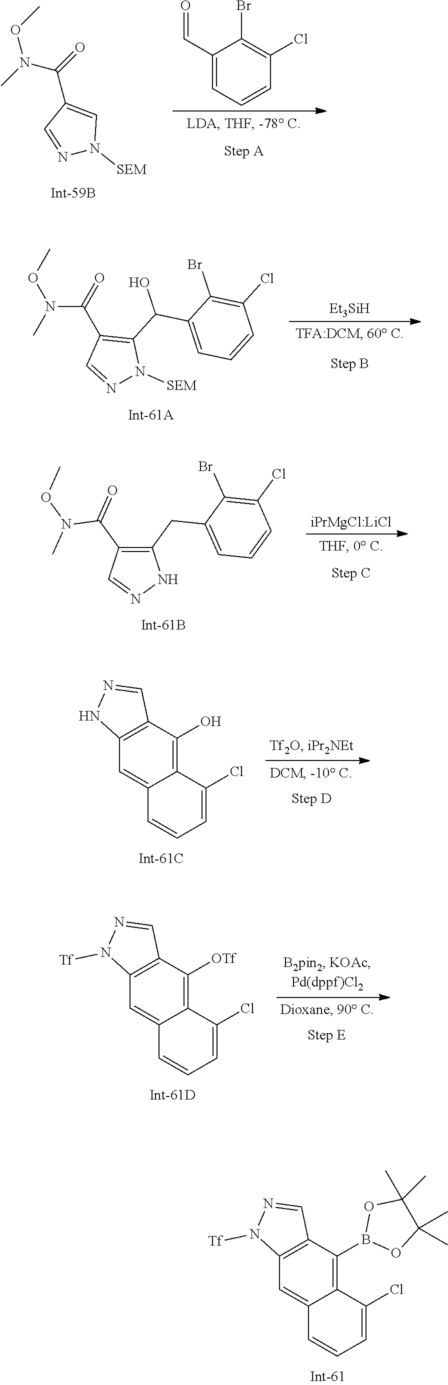

### Step A: 5-((2-bromo-3-chlorophenyl) (hydroxy)methyl)-N-methoxy-N-methyl-1-((2-(trimethylsilyl)ethoxy)methyl)-1H-pyrazole-4-carboxamide (Int-61A)

To a stirred solution of N-methoxy-N-methyl-1-((2-(trimethylsilyl)ethoxy)methyl)-1H-pyrazole-4-carboxamide (Int-59B) (5.00 g, 17.52 mmol) in THF (50 mL) was added lithium diisopropylamide (9.20 mL, 18.4 mmol, 2 M in THF) at −78° C. under N₂. The reaction was stirred at −78° C. for 0.5 h. Then 2-bromo-3-chlorobenzaldehyde (5.77 g, 26.3 mmol) was added at −78° C. and the reaction was stirred at −78° C. for 0.5 h. The mixture was quenched with sat. aq. NH₄Cl (20 mL) and extracted with EtOAc (3×40 mL), the organic layers were washed with sat. NaCl (20 mL), dried over sodium sulfate, and concentrated.

The residue was purified by flash silica gel chromatography (0 to 36% EtOAc in petroleum ether) to give 5-((2-bromo-3-chlorophenyl) (hydroxy)methyl)-N-methoxy-N-methyl-1-((2-(trimethylsilyl)ethoxy)methyl)-1H-pyrazole-4-carboxamide (Int-61A). MS (ESI) [M+H]⁺: m/z 504, 506.

### Step B: 5-(2-bromo-3-chlorobenzyl)-N-methoxy-N-methyl-1H-pyrazole-4-carboxamide (Int-61B)

To a solution of 5-((2-bromo-3-chlorophenyl) (hydroxy)methyl)-N-methoxy-N-methyl-1-((2-(trimethylsilyl)ethoxy)methyl)-1H-pyrazole-4-carboxamide (Int-61A) (6.30 g, 12.5 mmol) in DCM (40 mL) were added triethylsilane (80 mL, 500 mmol) and TFA (40 mL, 520 mmol). The mixture was stirred at 60° C. for 16 h. The reaction mixture was concentrated in vacuo and the residue was diluted with EtOAc (100 mL). The reaction mixture was basified to pH ˜8 with 2 N NaOH. The organic layer was washed with brine (2×20 mL), dried with sodium sulfate, filtered, and concentrated in vacuo, the residue was purified by flash silica gel chromatography (0˜25% EtOAc in petroleum ether) to give 5-(2-bromo-3-chlorobenzyl)-N-methoxy-N-methyl-1H-pyrazole-4-carboxamide (Int-61B). MS (ESI) [M+H]⁺: m/z 358, 360.

### Step C: 5-chloro-1H-benzo[f]indazol-4-ol (Int-61C)

To a stirred solution of 5-(2-bromo-3-chlorobenzyl)-N-methoxy-N-methyl-1H-pyrazole-4-carboxamide (Int-61B) (1.20 g, 3.35 mmol) in THF (30 mL) was added isopropylmagnesium chloride lithium chloride complex (10.3 mL, 13.4 mmol, 1.3 M in THF) at 0° C. under N₂. After the addition was finished, the reaction was stirred at 0° C. for 2 h. The mixture was adjusted to pH ˜5 with 1 M HCl, diluted with EtOAc (50 mL), then basified with sat. aq. NaHCO₃. The organic layer was washed with brine (20 mL), dried over sodium sulfate, filtered, and concentrated in vacuo. The residue was dispersed in with EtOAc (10 mL) and then stirred at room temperature for 10 min. The mixture was filtered and the solid was collected to give 5-chloro-1H-benzo[f]indazol-4-ol (Int-61C). MS (ESI) [M+H]⁺: m/z 219.

### Step D: 5-chloro-1-((trifluoromethyl) sulfonyl)-1H-benzo[f]indazol-4-yl trifluoromethanesulfonate (Int-61D)

To a solution of 5-chloro-1H-benzo[f]indazol-4-ol (Int-61C) (430 mg, 1.967 mmol) in DCM (20 mL) was added N,N-diisopropylethylamine (2.061 mL, 11.80 mmol) at −10° C., and the mixture was stirred for 10 min. Tf₂O (1.994 mL, 11.80 mmol) was added to the above mixture and the reaction was stirred at −10° C. for 1 h. The reaction was diluted with DCM (30 mL), washed with brine (10 mL), the organic layer was dried over sodium sulfate, and concentrated in vacuo. The residue was purified by flash silica gel chromatography (petroleum ether) to give 5-chloro-1-((trifluoromethyl) sulfonyl)-1H-benzo[f]indazol-4-yl trifluoromethanesulfonate (Int-61D). ¹H NMR (500 MHz, CDCl₃) δ: 8.72 (s, 1H), 8.55 (s, 1H), 8.04 (d, J=8.5 Hz, 1H), 7.79 (dd, J=7.4, 1.0 Hz, 1H), 7.62 (dd, J=8.3, 7.6 Hz, 1H).

### Step E: 5-chloro-4-(4,4,5,5-tetramethyl-1,3,2-dioxaborolan-2-yl)-1-((trifluoromethyl) sulfonyl)-1H-benzo[f]indazole (Int-61)

To a solution of 5-chloro-1-((trifluoromethyl) sulfonyl)-1H-benzo[f]indazol-4-yl trifluoromethanesulfonate (Int-61D) (1.2 g, 2.486 mmol) in dioxane (20 mL) was added bis(pinacolato)diboron (1.894 g, 7.46 mmol), KOAc (0.732 g, 7.46 mmol), and Pd(dppf)Cl₂ (0.182 g, 0.249 mmol) and the mixture was stirred at 90° C. for 4 h. The reaction mixture was diluted with EtOAc (30 mL) and the resulting mixture was filtered. The filtrate was concentrated in vacuo and the residue was purified by flash silica gel chromatography (petroleum ether/EtOAc=20/1) to give 5-chloro-4-(4,4,5,5-tetramethyl-1,3,2-dioxaborolan-2-yl)-1-((trifluoromethyl) sulfonyl)-1H-benzo[f]indazole (Int-61). MS (ESI) [M+H]⁺: m/z 461.

The compounds in the table below were synthesized using a similar procedure as described in the synthesis of Int-61 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-62 | 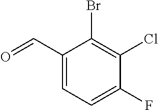 [SMILES: O=Cc1ccc(F)c(Cl)c1Br] | 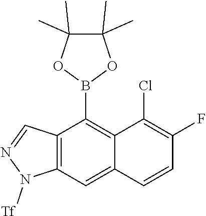 | 5-chloro-6-fluoro- 4-(4,4,5,5- tetramethyl-1,3,2- dioxaborolan-2-yl)- 1-((trifluoromethyl) sulfonyl)-1H- benzo[f]indazole | 479 |
| Int-63 | 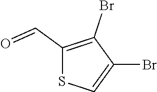 | 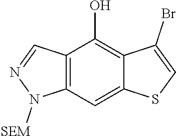 [SMILES: C[Si](C)(C)CCOCn1ncc2c(O)c3c(Br)csc3cc21] | 5-bromo-1-((2- (trimethylsilyl) ethoxy)methyl)-1H- thieno[3,2-f] indazol-4-ol; | 399 |

### Intermediate 64: (5-((triisopropylsilyl)ethynyl)-2-((2-(trimethylsilyl)ethoxy)methyl)-2H-thieno[3,2-f]indazol-4-yl)boronic acid (Int-64)

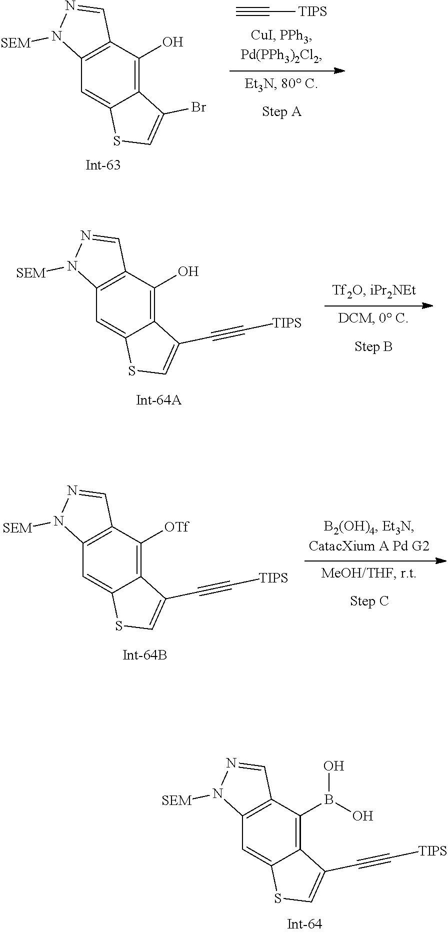

### Step A: 5-((triisopropylsilyl)ethynyl)-1-(2-(trimethylsilyl)ethoxy)methyl)-1H-thieno[3,2-f]indazol-4-ol (Int-64A)

To a solution of 5-bromo-1-((2-(trimethylsilyl)ethoxy)methyl)-1H-thieno[3,2-f]indazol-4-ol (Int-63) (620 mg, 1.552 mmol) in triethylamine (15 mL) was added ethynyltriisopropylsilane (425 mg, 2.329 mmol), copper (I) iodide (29.6 mg, 0.155 mmol), triphenylphosphine (40.7 mg, 0.155 mmol), and bis(triphenylphosphine) palladium (II) dichloride (54.5 mg, 0,078 mmol) at 25° C. under N₂. The mixture was stirred at 80° C. for 2 h. The reaction mixture was cooled and evaporated under reduced pressure to give the crude product. The crude product was purified by flash silica gel chromatography (0 to 10% EtOAc in petroleum ether) to give 5-((triisopropylsilyl)ethynyl)-1-((2-(trimethylsilyl)ethoxy)methyl)-1H-thieno[3,2-f]indazol-4-ol_(Int-64A). MS (ESI) [M+H]⁺: m/z 501.

### Step B: 5-((triisopropylsilyl)ethynyl)-1-((2-(trimethylsilyl)ethoxy)methyl)-1H-thieno[3,2-f]indazol-4-yl trifluoromethanesulfonate (Int-64B)

To a solution of 5-((triisopropylsilyl)ethynyl)-1-((2-(trimethylsilyl)ethoxy)methyl)-1H-thieno[3,2-f]indazol-4-ol (Int-64A) (442 mg, 0.883 mmol) in DCM (8 mL) was added N,N-diisopropylethylamine (0.462 mL, 2.65 mmol) at 0° C. and the mixture was stirred at 0° C. for 10 min. Tf₂O (0.224 mL, 1.324 mmol) was added and the reaction was stirred at 0° C. for 1 h. The reaction mixture was concentrated in vacuo to give crude product. The crude product was purified by flash silica gel chromatography (petroleum ether/EtOAc=10/1) to give 5-((triisopropylsilyl)ethynyl)-1-((2-(trimethylsilyl)ethoxy)methyl)-1H-thieno[3,2-f]indazol-4-yl trifluoromethanesulfonate (Int-64B). MS (ESI) [M+H]⁺: m/z 633.

### Step C: (5-((triisopropylsilyl)ethynyl)-1-((2-(trimethylsilyl)ethoxy)methyl)-1H-thieno[3,2-f]indazol-4-yl)boronic acid (Int-64)

To a solution of 5-((triisopropylsilyl)ethynyl)-1-((2-(trimethylsilyl)ethoxy)methyl)-1H-thieno[3,2-f]indazol-4-yl trifluoromethanesulfonate (Int-64B) (424 mg, 0.670 mmol) in MeOH/THF (5 mL, 3/1) was added hypodiboric acid (360 mg, 4.02 mmol), cataCXium A Pd G2 (44.8 mg, 0.067 mmol), and triethylamine (339 mg, 3.35 mmol). The mixture was stirred at 25° C. for 2 h under N₂. The mixture was diluted with water (3 mL) and extracted with EtOAc (3×). The organic layer was dried over sodium sulfate, filtered, and the filtrate was evaporated under reduced pressure. The crude product was purified by flash silica gel chromatography (0 to 35% EtOAc/petroleum ether) to give (5-((triisopropylsilyl)ethynyl)-1-((2-(trimethylsilyl)ethoxy)methyl)-1H-thieno[3,2-f]indazol-4-yl)boronic acid (Int-64). MS (ESI) [M+H]⁺: m/z 529.

### Intermediate 65:4-(5,5-dimethyl-1,3,2-dioxaborinan-2-yl)-6-fluoro-5-(triisopropylsilyl)ethynyl)-1-((2-(trimethylsilyl)ethoxy)methyl)-1H-thieno[3,2-f]indazole (Int-65)

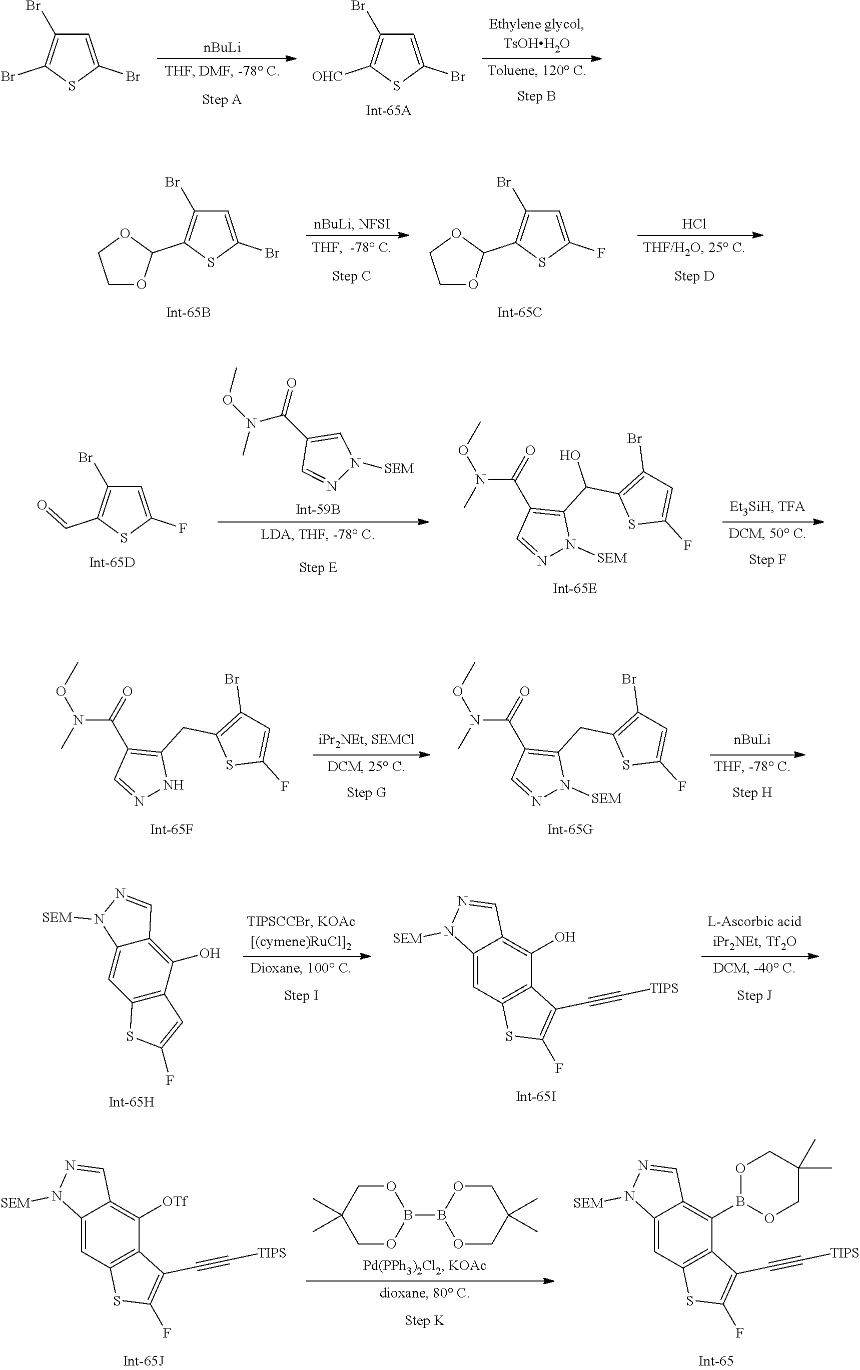

### Step A: 3,5-dibromothiophene-2-carbaldehyde (Int-65A)

To a solution of 2,3,5-tribromothiophene (100 g, 312 mmol) in THF (1.1 L) was added nBuLi (125 mL, 312 mmol, 2.5 M in hexane) dropwise at −78° C. under N₂. The mixture was stirred at −78° C. for 1 h, then DMF (29.0 mL, 374 mmol) was added to the reaction mixture. The mixture was stirred at −78° C. for 1.5 h. The mixture was quenched with ice water (500 mL), then treated with DCM (500 mL). The organic layer was separated and the aqueous layer was extracted with DCM (3×500 mL). The combined organic layers were dried over anhydrous sodium sulfate, filtered, and concentrated in vacuo to give a residue. The residue was diluted with petroleum ether (50 mL), stirred at 25° C. for 30 min, then filtered. The filter cake was washed with petroleum ether (3×30 mL) to give 3,5-dibromothiophene-2-carbaldehyde (Int-65A). MS (ESI) [M+H]⁺: m/z 269, 271.

### Step B: 2-(3,5-dibromothiophen-2-yl)-1,3-dioxolane (Int-65B)

To a solution of 3,5-dibromothiophene-2-carbaldehyde (48 g, 178 mmol) (Int-65A) in toluene (480 mL) was added ethylene glycol (39.7 mL, 711 mmol) and TsOH·H₂O (1.691 g, 8.89 mmol) at 25° C. The reaction was stirred at 120° C. for 16 h under N₂. The reaction mixture was cooled to room temperature and quenched with sat. aq. NaHCO₃ (200 mL). The reaction was treated with H₂O (100 mL) and DCM (300 mL). The organic layer was separated and the aqueous layer was extracted with DCM (3×300 mL). The combined organic layers were washed with brine (300 mL), dried over anhydrous sodium sulfate, filtered, and concentrated in vacuo to give a crude residue. The residue was purified by flash silica gel chromatography (3% EtOAc in petroleum ether) to give 2-(3,5-dibromothiophen-2-yl)-1,3-dioxolane (Int-65B). MS (ESI) [M+H]⁺: m/z 313, 315.

### Step C: 2-(3-bromo-5-fluorothiophen-2-yl)-1,3-dioxolane (Int-65C)

To a solution of 2-(3,5-dibromothiophen-2-yl)-1,3-dioxolane (Int-65B) (40.66 g, 129 mmol) in THF (400 mL) was added nBuLi (51.8 mL, 129 mmol, 2.5 M in hexane) dropwise at −78° C. under N₂. The mixture was stirred at −78° C. for 30 min. A solution of NFSI (82 g, 259 mmol) in THF (200 mL) was added to the reaction mixture. The mixture was stirred at −78° C. for 10 min under N₂. The mixture was quenched with sat. aq. NH₄Cl (400 mL). The mixture was treated with H₂O (100 mL) and EtOAc (400 mL). The organic layer was separated and the aqueous layer was extracted with EtOAc (3×400 mL). The combined organic layers were washed with brine (400 mL), dried over anhydrous sodium sulfate, filtered, and concentrated. The residue was purified by flash silica gel chromatography (0 to 5% EtOAc in petroleum ether) to give 2-(3-bromo-5-fluorothiophen-2-yl)-1,3-dioxolane (Int-65C). MS (ESI) [M+H]⁺: m/z 253, 255.

### Step D: 3-bromo-5-fluorothiophene-2-carbaldehyde (Int-64D)

A solution of 2-(3-bromo-5-fluorothiophen-2-yl)-1,3-dioxolane (Int-65C) (14.0 g, 55.3 mmol) in THF (300 mL) and 1 M HCl (100 mL) was stirred at 25° C. for 16 h under N₂. The reaction was treated with H₂O (100 mL) and EtOAc (100 mL). The organic layer was separated and the aqueous layer was extracted with EtOAc (3×100 mL). The combined organic layers were washed with brine (100 mL), dried over anhydrous sodium sulfate, filtered, and concentrated in vacuo to give a residue. The crude material was purified by flash silica gel chromatography (0 to 5% DCM in petroleum ether) to give the crude product. The crude was repurified by reverse-phase MPLC (C18 column, 0 to 35% MeCN in H₂O (0.5% % TFA) to give 3-bromo-5-fluorothiophene-2-carbaldehyde (Int-65D). MS (ESI) [M+H]⁺: m/z 209, 211.

### Step E: 5-((3-bromo-5-fluorothiophen-2-yl) (hydroxy)methyl)-N-methoxy-N-methyl-1-((2-(trimethylsilyl)ethoxy)methyl)-1H-pyrazole-4-carboxamide (Int-65E)

To a stirred solution of N-methoxy-N-methyl-1-((2-(trimethylsilyl)ethoxy)methyl)-1H-pyrazole-4-carboxamide (Int-59B) (13.7 g, 48.0 mmol) in THF (130 mL) was added LDA (27.6 mL, 55.2 mmol, 1 M in THF) dropwise at −78° C. under N₂. The reaction was stirred at −78° C. for 0.5 h, 3-bromo-5-fluorothiophene-2-carbaldehyde (Int-65D) (5.02 g, 24.0 mmol) in THF (25 mL) was added at −78° C. The reaction was stirred at −78° C. for 10 min. The mixture was quenched with sat. aq. NH₄Cl (50 mL). The reaction was treated with H₂O (50 mL) and EtOAc (100 mL). The organic layer was separated and the aqueous layer was extracted with EtOAc (3×50 mL). The combined organic layers were washed with brine (50 mL), dried over anhydrous sodium sulfate, filtered, and concentrated in vacuo to give the crude product. The crude was purified by flash silica gel chromatography (0 to 20% EtOAc in petroleum ether) to give 5-((3-bromo-5-fluorothiophen-2-yl)(hydroxy)methyl)-N-methoxy-N-methyl-1-((2-(trimethylsilyl)ethoxy)methyl)-1H-pyrazole-4-carboxamide (Int-65E). ¹H NMR (400 MHz, CDCl₃) δ 8.08 (s, 1H), 7.51 (d, J=11.2 Hz, 1H), 6.36-6.42 (m, 2H), 5.54-5.70 (m, 2H), 3.77 (s, 3H), 3.47-3.60 (m, 2H), 3.44 (s, 3H), 0.79-0.93 (m, 2H), 0.00 (s, 9H).

### Step F: 5-((3-bromo-5-fluorothiophen-2-yl)methyl)-N-methoxy-N-methyl-1H-pyrazole-4-carboxamide (Int-65F)

To a solution of 5-((3-bromo-5-fluorothiophen-2-yl) (hydroxy)methyl)-N-methoxy-N-methyl-1-((2-(trimethylsilyl)ethoxy)methyl)-1H-pyrazole-4-carboxamide (Int-65E) (7.9 g, 16 mmol) in DCM (20 mL) was added Et₃SIH (40 mL) and TFA (20 mL) at 25° C. The reaction was stirred at 50° C. for 2 h under N₂. The solvent was cooled to room temperature and evaporated under reduced pressure to give the crude product. The crude was diluted with EtOAc (80 mL) and adjusted to pH ˜7 with sat. aq. NaHCO₃ (80 mL). The organic layer was separated and the aqueous layer was extracted with EtOAc (3×50 mL). The combined organic layers were washed with brine (50 mL), dried over anhydrous sodium sulfate, filtered, and concentrated in vacuo to give the crude product. The crude product was suspended in petroleum ether (50 mL), filtered, and washed with petroleum ether (20 mL) to give 5-((3-bromo-5-fluorothiophen-2-yl)methyl)-N-methoxy-N-methyl-1H-pyrazole-4-carboxamide (Int-65F). MS (ESI) [M+H]⁺: m/z 348, 350.

### Step G: 5-((3-bromo-5-fluorothiophen-2-yl)methyl)-N-methoxy-N-methyl-1-((2-(trimethylsilyl)ethoxy)methyl)-1H-pyrazole-4-carboxamide (Int-65G)

To a stirred solution of 5-((3-bromo-5-fluorothiophen-2-yl)methyl)-N-methoxy-N-methyl-1H-pyrazole-4-carboxamide (Int-65F) (4.63 g, 13.3 mmol) in DCM (60 mL) was added N,N-diisopropylethylamine (4.63 mL, 26.6 mmol) and SEMCl (3.69 mL, 20 mmol) at 20° C. under N₂. The reaction was stirred at 20° C. for 12 h. The reaction was treated with H₂O (50 mL) and DCM (60 mL). After filtration, the organic layer was separated and the aqueous layer was extracted with EtOAc (3×60 mL). The combined organic layers were washed with sat. aq. NaHCO₃ (50 mL), dried over anhydrous sodium sulfate, filtered, and concentrated in vacuo to give a residue. The residue was purified by flash silica gel chromatography (0 to 20% EtOAc in petroleum ether) to give 5-((3-bromo-5-fluorothiophen-2-yl)methyl)-N-methoxy-N-methyl-1-((2-(trimethylsilyl)ethoxy)methyl)-1H-pyrazole-4-carboxamide (Int-65G). MS (ESD) [M+H]⁺: m/z 478, 480.

### Step H: 6-fluoro-1-((2-(trimethylsilyl)ethoxy)methyl)-1H-thieno[3,2-f]indazol-4-ol (Int-65H)

To a solution of 5-((3-bromo-5-fluorothiophen-2-yl)methyl)-N-methoxy-N-methyl-1-((2-(trimethylsilyl)ethoxy)methyl)-1H-pyrazole-4-carboxamide (Int-65G) (1.60 g, 3.34 mmol) in THF (20 mL) was added nBuLi (2.006 mL, 5.02 mmol) at −78° C. dropwise under N₂. The reaction was stirred at −78° C. for 0.5 h. The mixture was quenched with sat. aq. NH₄Cl (20 mL). The mixture was treated with H₂O (20 mL) and EtOAc (20 mL). The organic layer was separated and the aqueous layer was extracted with EtOAc (3×20 mL). The combined organic layers were washed with brine (20 mL), dried over anhydrous sodium sulfate, filtered, and concentrated to give the crude. The crude residue was purified by flash silica gel chromatography (0 to 20% THF in petroleum ether) to provide 6-fluoro-1-((2-(trimethylsilyl)ethoxy)methyl)-1H-thieno[3,2-f]indazol-4-ol (Int-65H). MS (ESI) [M+H]⁺: m/z 339.

### Step I: 6-fluoro-5-((triisopropylsilyl)ethynyl)-1-((2-(trimethylsilyl)ethoxy)methyl)-1H-thieno[3,2-f]indazol-4-ol (Int-65I)

To a solution of 6-fluoro-1-((2-(trimethylsilyl)ethoxy)methyl)-1H-thieno[3,2-f]indazol-4-ol (Int-65H) (900 mg, 2.66 mmol) and [(cymene) RuCl]₂ (814 mg, 1.330 mmol) in dioxane (36 mL) was added (bromoethynyl)triisopropylsilane (2.50 g, 9.57 mmol) and KOAc (992 mg, 10.1 mmol) at 25° C. under N₂. The mixture was stirred at 100° C. for 2 h. The mixture was cooled and diluted with EtOAc (20 mL). The resulting mixture was filtered and concentrated in vacuo to give the crude product. The crude was purified by flash silica gel chromatography (0 to 1% EtOAc in DCM) to give 6-fluoro-5-((triisopropylsilyl)ethynyl)-1-((2-(trimethylsilyl)ethoxy)methyl)-1H-thieno[3,2-f]indazol-4-ol (Int-65I). MS (ESI) [M+H]⁺: m/z 519.

### Step J: 6-fluoro-5-((triisopropylsilyl)ethynyl)-1-((2-(trimethylsilyl)ethoxy)methyl)-1H-thieno[3,2-f]indazol-4-yl trifluoromethanesulfonate (Int-65J)

To a solution of 6-fluoro-5-((triisopropylsilyl)ethynyl)-1-((2-(trimethylsilyl)ethoxy)methyl)-1H-thieno[3,2-f]indazol-4-ol (Int-65I) (1.00 g, 1.927 mmol) and L-ascorbic acid (0.017 g, 0.096 mmol) in DCM (20 mL) was added N,N-diisopropylethylamine (2.020 mL, 11.56 mmol) at −40° C. Tf₂O (0.98 mL, 5.78 mmol) was added to the above mixture at −40° C. The reaction was stirred at −40° C. for 0.5 h under N₂. The mixture was concentrated in vacuo to give a residue. The residue was purified by flash silica gel chromatography (0 to 10% EtOAc in petroleum ether) to give 6-fluoro-5-((triisopropylsilyl)ethynyl)-1-((2-(trimethylsilyl)ethoxy)methyl)-1H-thieno[3,2-f]indazol-4-yl trifluoromethanesulfonate (Int-65J). MS (ESI) [M+H]⁺: m/z 651.

### Step K: 4-(5,5-dimethyl-1,3,2-dioxaborinan-2-yl)-6-fluoro-5-((triisopropylsilyl)ethynyl)-1-((2-(trimethylsilyl)ethoxy)methyl)-1H-thieno[3,2-f]indazole (Int-65)

To a solution of 6-fluoro-5-((triisopropylsilyl)ethynyl)-1-((2-(trimethylsilyl)ethoxy)methyl)-1H-thieno[3,2-f]indazol-4-yl trifluoromethanesulfonate (Int-65J) (900 mg, 1.38 mmol) in dioxane (30 mL) was added KOAc (407 mg, 4.15 mmol) and bis(neopentyl glycolato)diboron (937 mg, 4.15 mmol) at 25° C. under N₂. The reaction was stirred at 25° C. for 10 min. Pd(PPh₃)₂Cl₂ (97 mg, 0.14 mmol) was added to the reaction under N₂. The reaction was stirred at 80° C. for 3 h. The mixture was cooled and diluted with EtOAc (20 mL). The resulting mixture was filtered and concentrated in vacuo to give the crude product. The residue was purified by flash silica gel chromatography (0 to 10% EtOAc in petroleum ether) to give 4-(5,5-dimethyl-1,3,2-dioxaborinan-2-yl)-6-fluoro-5-((triisopropylsilyl)ethynyl)-1-((2-(trimethylsilyl)ethoxy)methyl)-1H-thieno[3,2-f]indazole (Int-65). MS (ESI) [M+H]⁺: m/z 615.

### Intermediate 66: (5-methyl-1-(tetrahydro-2H-pyran-2-yl)-1,5-dihydropyrrolo[2,3-f]indazol-4-yl)boronic acid (Int-66)

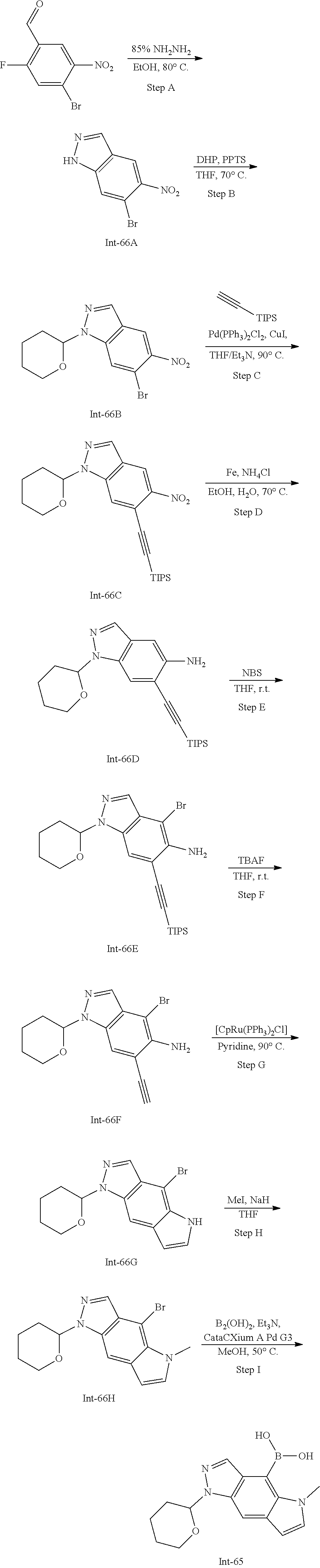

### Step A: 6-bromo-5-nitro-1H-indazole (Int-66A)

To a solution of 4-bromo-2-fluoro-5-nitrobenzaldehyde (4.0 g, 16.13 mmol) in EtOH (40 mL) was added hydrazine hydrate (4.8 g, 81 mmol, 85% in H₂O) at 25° C. The mixture was stirred at 80° C. for 2 h. The solvent was removed under reduced pressure and the crude was diluted with EtOAc (100 mL). The mixture was washed with H₂O (15 mL) and saturated brine (15 mL). The organic layer was dried over sodium sulfate, filtered, and the solvent was removed under reduced pressure to give 6-bromo-5-nitro-1H-indazole (Int-66A). MS (ESI) [M+H]⁺: m/z 242, 244.

### Step B: 6-bromo-5-nitro-1-(tetrahydro-2H-pyran-2-yl)-1H-indazole (Int-66B)

To a solution of 6-bromo-5-nitro-1H-indazole (Int-66A) (3.50 g, 14.5 mmol) in THF (35 mL) was added DHP (3.97 mL, 43.4 mmol) and PPTS (0.623 g, 3.62 mmol) at 25° C. The mixture was stirred at 70° C. for 5 h. The reaction mixture was cooled, diluted with H₂O (10 mL), extracted with EtOAc (2×50 mL), dried over sodium sulfate, filtered, and concentrated in vacuo to give the crude product. The residue was purified by flash silica gel chromatography (15% EtOAc in petroleum ether) to give 6-bromo-5-nitro-1-(tetrahydro-2H-pyran-2-yl)-1H-indazole (Int-66B). MS (ESI) [M+H]⁺: m/z 326, 328.

### Step C: 5-nitro-1-(tetrahydro-2H-pyran-2-yl)-6-(triisopropylsilyl)ethynyl)-1H-indazole (Int-66C)

To a solution of 6-bromo-5-nitro-1-(tetrahydro-2H-pyran-2-yl)-1H-indazole (Int-66B) (4.2 g, 12.9 mmol) in THF (50 mL) and Et₃N (50 mL) were added ethynyltriisopropylsilane (9.39 g, 51.5 mmol), dichlorobis(triphenylphosphine) palladium (II) (1.36 g, 1.932 mmol), and copper (I) iodide (0.491 g, 2.58 mmol) at room temperature. The reaction vessel was sealed and the mixture was stirred at 90° C. for 12 h under N₂. The mixture was cooled to room temperature, quenched with H₂O (25 mL), extracted with EtOAc (3×50 mL). The combined organic layers were dried over sodium sulfate, filtered, and concentrated in vacuo to give the crude product. The residue was purified by flash silica gel chromatography (0 to 15% EtOAc in petroleum ether) to give 5-nitro-1-(tetrahydro-2H-pyran-2-yl)-6-((triisopropylsilyl)ethynyl)-1H-indazole (Int-66C). MS (ESI) [M+H]⁺: m/z 428.

### Step D: 1-(tetrahydro-2H-pyran-2-yl)-6-((triisopropylsilyl)ethynyl)-1H-indazol-5-amine (Int-66D)

To a solution of 5-nitro-1-(tetrahydro-2H-pyran-2-yl)-6-((triisopropylsilyl)ethynyl)-1H-indazole (Int-66C) (5.00 g, 11.7 mmol) in EtOH (50 mL) and H₂O (10 mL) were added NH₄Cl (1.88 g, 35.1 mmol) and iron powder (1.96 g, 35.1 mmol) at 25° C. The mixture was stirred at 70° C. for 2 h. The solvent was removed under reduced pressure and the residue was diluted with EtOAc (50 mL). The mixture was filtered, washed with H₂O (2×5 mL). The organic layer was dried over sodium sulfate, filtered, and the solvent was removed under reduced pressure. The crude product was purified by flash silica gel chromatography (0 to 10% EtOAc in petroleum ether) to provide 1-(tetrahydro-2H-pyran-2-yl)-6-((triisopropylsilyl)ethynyl)-1H-indazol-5-amine (Int-66D). MS (ESI) [M+H]⁺: m/z 398.

### Step E: 4-bromo-1-(tetrahydro-2H-pyran-2-yl)-6-((triisopropylsilyl)ethynyl)-1H-indazol-5-amine (Int-66E)

To a solution of 1-(tetrahydro-2H-pyran-2-yl)-6-((triisopropylsilyl)ethynyl)-1H-indazol-5-amine (Int-66D) (4.30 g, 10.8 mmol) in THF (50 mL) was added NBS (2.12 g, 11.90 mmol) at 25° C. The mixture was stirred at 25° C. for 1 h. The mixture was diluted with water (10 mL) and extracted with EtOAc (2×50 mL). The combined organic layers were dried over sodium sulfate, filtered, and the solvent was removed under reduced pressure. The crude was purified by flash silica gel chromatography (0 to 2% EtOAc in petroleum ether) to give 4-bromo-1-(tetrahydro-2H-pyran-2-yl)-6-((triisopropylsilyl)ethynyl)-1H-indazol-5-amine (Int-66E). MS (ESI) [M+H]⁺: m/z 476, 478.

### Step F: 4-bromo-6-ethynyl-1-(tetrahydro-2H-pyran-2-yl)-1H-indazol-5-amine (Int-66F)

To a solution of 4-bromo-1-(tetrahydro-2H-pyran-2-yl)-6-((triisopropylsilyl)ethynyl)-1H-indazol-5-amine (Int-66E) (4.20 g, 8.81 mmol) in THF (45 mL) was added TBAF (26.4 mL, 26.4 mmol, 1 M in THF) at 25° C. The mixture was stirred at 25° C. for 2 h. The reaction was quenched with sat. aq. NH₄Cl (10 mL) and extracted with EtOAc (2×40 mL). The combined organic layers were dried with sodium sulfate, filtered, and the solvent was removed under reduced pressure. The crude product was purified by flash silica gel chromatography (0 to 15% EtOAc in petroleum ether) to give 4-bromo-6-ethynyl-1-(tetrahydro-2H-pyran-2-yl)-1H-indazol-5-amine (Int-66F). MS (ESD) [M+H]⁺: m/z 320, 322.

### Step G: 4-bromo-1-(tetrahydro-2H-pyran-2-yl)-1,5-dihydropyrrolo[2,3-f]indazole (Int-66G)

To a solution of 4-bromo-6-ethynyl-1-(tetrahydro-2H-pyran-2-yl)-1H-indazol-5-amine (Int-66F) (1.80 g, 5.62 mmol) in pyridine (37.4 mL) was added chlorocyclopentadienylbis(triphenylphosphine) ruthenium (II) (0.408 g, 0.562 mmol) at 25° C. under N₂ and the mixture was stirred at 90° C. for 2 h. The reaction was cooled to room temperature and quenched with water (50 mL). The reaction mixture was extracted with EtOAc (2×40 mL). The combined organic layers were dried over sodium sulfate, filtered, and the solvent was removed under reduced pressure. The crude reaction mixture was purified by flash silica gel chromatography (0 to 10% EtOAc in petroleum ether) to give 4-bromo-1-(tetrahydro-2H-pyran-2-yl)-1,5-dihydropyrrolo[2,3-f]indazole (Int-66G). MS (ESI) [M+H]⁺: m/z 320, 322.

### Step H: 4-bromo-5-methyl-1-(tetrahydro-2H-pyran-2-yl)-1,5-dihydropyrrolo[2,3-f]indazole (Int-66H

To a solution of 4-bromo-1-(tetrahydro-2H-pyran-2-yl)-1,5-dihydropyrrolo[2,3-f]indazole (Int-66G) (1.50 g, 4.68 mmol) in THF (15 mL) was added NaH (0.562 g, 14.1 mmol, 60 wt % in mineral oil) at 0° C. The mixture was stirred at 0° C. for 20 min. Iodomethane (0.44 mL, 7.03 mmol) was added and the mixture was stirred at 0° C. for 1 h. The reaction was quenched with water (50 mL), extracted with EtOAc (2×20 mL). The combined organic layers were dried over sodium sulfate, filtered, and the solvent was removed under reduced pressure. The crude was purified by flash silica gel chromatography (0 to 5% EtOAc in petroleum ether) to give 4-bromo-5-methyl-1-(tetrahydro-2H-pyran-2-yl)-1,5-dihydropyrrolo[2,3-f]indazole (Int-66H). MS (ESI) [M+H]⁺: m/z 334, 336.

### Step 1:(5-methyl-1-(tetrahydro-2H-pyran-2-yl)-1,5-dihydropyrrolo[2,3-f]indazol-4-ylboronic acid (Int-66)

To a solution of 4-bromo-5-methyl-1-(tetrahydro-2H-pyran-2-yl)-1,5-dihydropyrrolo[2,3-f]indazole (Int-66H) (300 mg, 0.898 mmol) in MeOH (10 mL) was added TEA (0.375 mL, 2.69 mmol), tetrahydroxydiboron (322 mg, 3.59 mmol), and cataCXium A Pd G3 (30.0 mg, 0.045 mmol) at 20° C. under N₂. The mixture was stirred at 50° C. for 1 h. The reaction mixture was concentrated in vacuo and the residue was purified by flash silica gel chromatography (0 to 10% MeOH in DCM) to provide (5-methyl-1-(tetrahydro-2H-pyran-2-yl)-1,5-dihydropyrrolo[2,3-f]indazol-4-yl)boronic acid (Int-66). MS (ESI) [M+H]⁺: m/z 300.

### Intermediate 67:4-bromo-5-cyclopropyl-6-ethyl-1-(tetrahydro-2H-pyran-2-yl)-1H-indazole (Int-67)

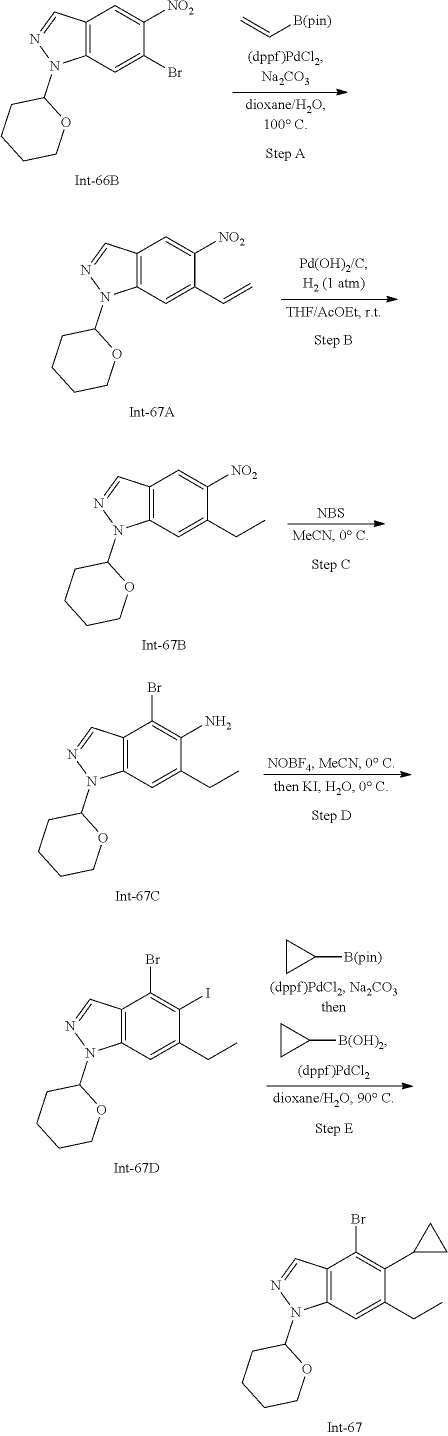

### Step A: 5-nitro-1-(tetrahydro-2H-pyran-2-yl)-6-vinyl-1H-indazole (Int-67A)

A mixture of 6-bromo-5-nitro-1-(tetrahydro-2H-pyran-2-yl)-1H-indazole (Int-66B) (980 mg, 3.00 mmol), [1,1′ bis(diphenylphosphino)ferrocene]dichloropalladium (II) (122 mg, 0.149 mmol), 4,4,5,5-tetramethyl-2-vinyl-1,3,2-dioxaborolane (1.00 mL, 5.84 mmol), and Na₂CO₃ (3.00 mL, 6.00 mmol, 2 M in H₂O) in dioxane (12 mL) was stirred at 100° C. under N₂ for 14 h. The reaction was cooled to room temperature and diluted with water (5 mL) and EtOAc (5 mL). The layers were shaken and separated and the organic phase was washed with brine (15 mL). The organic phase was dried over Na₂SO₄, filtered, and concentrated under reduced pressure. The crude residue was purified by silica gel chromatography (hexane/EtOAc=95/5 to 70/30) to afford 5-nitro-1-(tetrahydro-2H-pyran-2-yl)-6-vinyl-1H-indazole (Int-67A). MS (ESI) [M+H]⁺: m/z 274.

### Step B: 6-ethyl-1-(tetrahydro-2H-pyran-2-yl)-1H-indazol-5-amine (Int-67B)

5-nitro-1-(tetrahydro-2H-pyran-2-yl)-6-vinyl-1H-indazole (Int-67A) (721 mg, 2.63 mmol) was dissolved in THF (7 mL) and EtOAc (7 mL). The reaction was purged with nitrogen and palladium hydroxide on carbon (500 mg) was added. The atmosphere was replaced with hydrogen and the reaction mixture was stirred at room temperature for 5 h. The reaction was filtered and concentrated in vacuo to afford 6-ethyl-1-(tetrahydro-2H-pyran-2-yl)-1H-indazol-5-amine (Int-67B), which was used directly in the next step without further purification. MS (ESI) [M+H]⁺: m/z 246.

### Step C: 4-bromo-6-ethyl-1-(tetrahydro-2H-pyran-2-yl)-1H-indazol-5-amine (Int-670)

To a solution of 6-ethyl-1-(tetrahydro-2H-pyran-2-yl)-1H-indazol-5-amine (Int-67B) (648 mg, 2.64 mmol) in MeCN (10 mL) was added NBS (500 mg, 2.81 mmol) at 0° C. The reaction mixture was stirred at 0° C. for 10 min. The reaction mixture was quenched with sat. aq. NaHCO₃ and aq. Na₂S₂O₃. The reaction was diluted with EtOAc. The organic layer was separated, washed with brine, dried over Na₂SO₄, filtered, and the solvent was evaporated under reduced pressure. The residue was purified by silica gel chromatography (0 to 100% EtOAc in hexanes) to afford 4-bromo-6-ethyl-1-(tetrahydro-2H-pyran-2-yl)-1H-indazol-5-amine (Int-67C). MS (ESI) [M+H]⁺: m/z 324, 326.

### Step D: 4-bromo-6-ethyl-5-iodo-1-(tetrahydro-2H-pyran-2-yl)-1H-indazole (Int-67D)

To a solution of 4-bromo-6-ethyl-1-(tetrahydro-2H-pyran-2-yl)-1H-indazol-5-amine (Int-67C) (318 mg, 0.981 mmol) in MeCN (5 mL) was added nitrosyl tetrafluoroborate (160 mg, 1.37 mmol) at 0° C. and stirred for 5 min. The mixture was added to a solution of KI (5.00 g, 30.1 mmol) in water (10 mL) at 0° C., stirred for 5 min. The mixture was quenched with sat. aq. NaHCO₃ and aq. Na₂S₂O₃. The aqueous layer was extracted with EtOAc and the organic layer was dried over Na₂SO₄, filtered, and the solvent was evaporated under reduced pressure. The residue was purified by silica gel chromatography (0 to 100% EtOAc in hexanes) to afford 4-bromo-6-ethyl-5-iodo-1-(tetrahydro-2H-pyran-2-yl)-1H-indazole (Int-67D) MS (ESI) [M+H]⁺: m/z 435, 437.

### Step E: 4-bromo-5-cyclopropyl-6-ethyl-1-(tetrahydro-2H-pyran-2-yl)-1H-indazole (Int-67)

A mixture of 4-bromo-6-ethyl-5-iodo-1-(tetrahydro-2H-pyran-2-yl)-1H-indazole (Int-67D) (345 mg, 0.792 mmol), [1,1′ bis(diphenylphosphino)ferrocene]dichloropalladium (II) (116 mg, 0.159 mmol), 2-cyclopropyl-4,4,5,5-tetramethyl-1,3,2-dioxaborolane (70 mg, 0.82 mmol), Na₂CO₃ (1.60 mL, 3.20 mmol, 2 M in H₂O) in dioxane (8 mL) was stirred at 90° C. under N₂ overnight. Additional [1,1′ bis(diphenylphosphino)ferrocene]dichloropalladium (II) (116 mg, 0.159 mmol) and 2-cyclopropylboronic acid (70 mg, 0.82 mmol) were added and the mixture was stirred at 95° C. under N₂ overnight. The mixture was filtered, and the filtrate was concentrated under reduced pressure. The residue obtained was purified by silica gel chromatography (hexane/EtOAc=95/5 to 70/30) to afford 4-bromo-5-cyclopropyl-6-ethyl-1-(tetrahydro-2H-pyran-2-yl)-1H-indazole (Int-67). MS (ESI) [M+H]⁺: m/z 349, 351.

The compound in the table below was synthesized using a similar procedure as described in the synthesis of Int-67 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-68 | 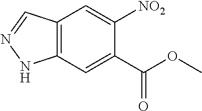 [SMILES: COC(=O)c1cc2[nH]ncc2cc1[N+](=O)[O-]] | 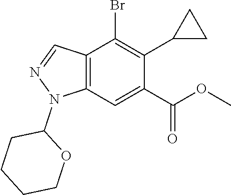 [SMILES: COC(=O)c1cc2c(cnn2C2CCCCO2)c(Br)c1C1CC1] | Methyl 4-bromo-5- cyclopropyl-1- (tetrahydro-2H- pyran-2-yl)-1H- indazole-6- carboxylate | 379, 381 |

### Intermediate 69: (5-cyclopropyl-6-(methoxymethyl)-1-(tetrahydro-2H-pyran-2-yl)-1H-indazol-4-yl)boronic acid (Int-69)

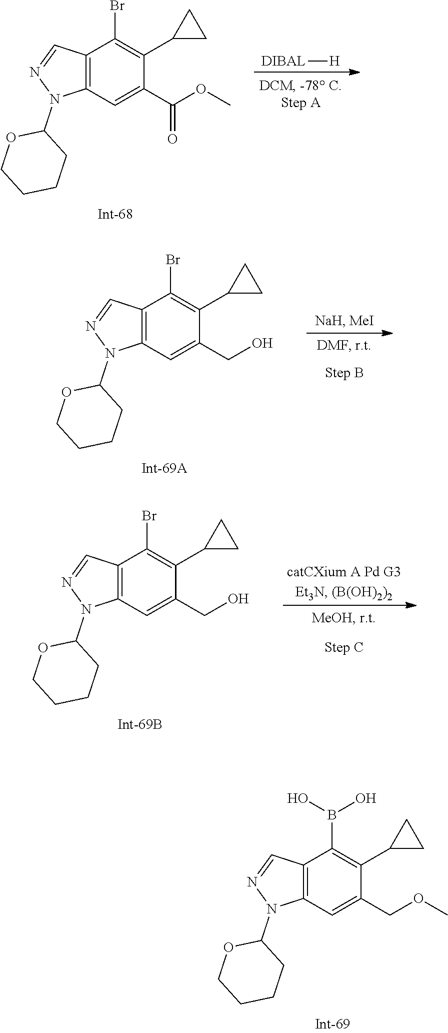

### Step A: (4-bromo-5-cyclopropyl-1-(tetrahydro-2H-pyran-2-yl)-1H-indazol-6-yl) methanol (Int-69A)

To a solution of methyl 4-bromo-5-cyclopropyl-1-(tetrahydro-2H-pyran-2-yl)-1H-indazole-6-carboxylate (Int-68) (114 mg, 0.301 mmol) in DCM (5 mL) was added diisobutylaluminum hydride (0.800 mL, 0.816 mmol, 1.02 M in hexane) at −78° C. The mixture was stirred at −78° C. for 1 h. The mixture was quenched with sat. aq. Rochelle's salt and diluted with EtOAc. The mixture was warmed to the ambient temperature and stirred for 3 h. The organic layer was separated, washed with brine, and dried over Na₂SO₄. The dried solution was filtered and the filtrate was concentrated under reduced pressure. The residue obtained was purified by silica gel chromatography (hexane/EtOAc=90/10 to 0/100) to afford (4-bromo-5-cyclopropyl-1-(tetrahydro-2H-pyran-2-yl)-1H-indazol-6-yl) methanol (Int-69A), MS (ESI) [M+H]⁺: m/z 351, 353.

### Step B: 4-bromo-5-cyclopropyl-6-(methoxymethyl)-1-(tetrahydro-2H-pyran-2-yl)-1H-indazole (Int-69B)

To the mixture of (4-bromo-5-cyclopropyl-1-(tetrahydro-2H-pyran-2-yl)-1H-indazol-6-yl) methanol (Int-69A) (64 mg, 0.18 mmol) and iodomethane (0.040 mL, 0.643 mmol) in DMF (1 mL) was added sodium hydride (20 mg, 0.500 mmol, 60 wt % in mineral oil). The mixture was diluted with water and EtOAc. The organic layer was separated, washed with brine, and dried over Na₂SO₄. The dried solution was filtered and the filtrate was concentrated under reduced pressure to afford 4-bromo-5-cyclopropyl-6-(methoxymethyl)-1-(tetrahydro-2H-pyran-2-yl)-1H-indazole (Int-69B), which was used directly in the next step without further purification. MS (ESI) [M+H]⁺: m/z 365, 367.

### Step C: (5-cyclopropyl-6-(methoxymethyl)-1-(tetrahydro-2H-pyran-2-yl)-1H-indazol-4-yl)boronic acid (Int-69)

To a solution of 4-bromo-5-cyclopropyl-6-(methoxymethyl)-1-(tetrahydro-2H-pyran-2-yl)-1H-indazole (Int-69B) in MeOH (2 mL) were added tetrahydroxydiboron (60 mg, 0.67 mmol)), triethylamine (0.140 mL, 1.01 mmol) and cataCXium A Pd G3 (0.010 g, 0.014 mmol). The mixture was stirred at room temperature overnight under N₂. EtOAc, CHCl₃ and 10% aq. H₃PO₄ were added, the organic layer was separated, washed with brine, and dried over Na₂SO₄. The dried solution was filtered and the filtrate was concentrated under reduced pressure. The residue obtained was purified by silica gel chromatography (CHCl₃/ethanol=100/0 to 80/20) to afford (5-cyclopropyl-6-(methoxymethyl)-1-(tetrahydro-2H-pyran-2-yl)-1H-indazol-4-yl)boronic acid (Int-69). MS (ESI) [M+H]⁺: m/z 331.

The compound in the table below were synthesized using a similar procedure as described in the synthesis of Int-69 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-70 | 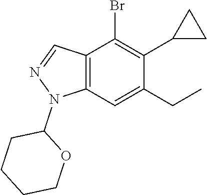 [SMILES: CCc1cc2c(cnn2C2CCCCO2)c(Br)c1C1CC1] Int-67 | 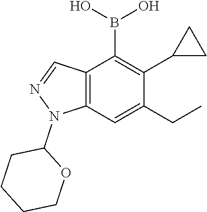 [SMILES: CCc1cc2c(cnn2C2CCCCO2)c(B(O)O)c1C1CC1] | (5-cyclopropyl-6- ethyl-1- (tetrahydro-2H- pyran-2-yl)-1H- indazol-4-yl) boronic acid | 315 |

### Intermediate 71: (6-cyano-5-cyclopropyl-1-(tetrahydro-2H-pyran-2-yl)-1H-indazol-4-yl)boronic acid (Int-71)

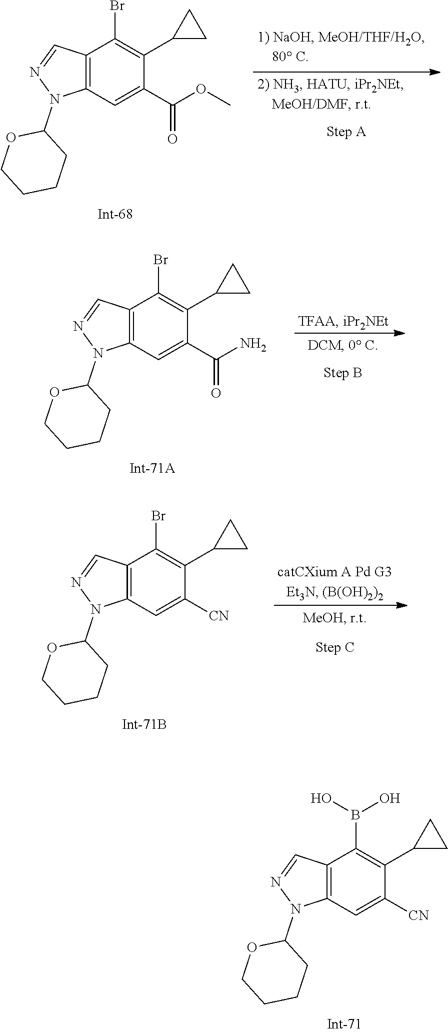

### Step A: 4-bromo-5-cyclopropyl-1-(tetrahydro-2H-pyran-2-yl)-1H-indazole-6-carboxamide (Int-71A)

NaOH (1.5 mL, 9.0 mmol, 6M in H₂O) was added to a solution of methyl 4-bromo-5-cyclopropyl-1-(tetrahydro-2H-pyran-2-yl)-1H-indazole-6-carboxylate (Int-68) (178 mg, 0.469 mmol) in THF (1.0 mL) and MeOH (1.5 mL). The reaction mixture was heated to 80° C. in a microwave reactor for 90 min. The reaction was cooled to room temperature and EtOAc, CHCl₃, and H₃PO₄ (10% aq.) were added. The layers were separated and the organic phase was washed with brine, dried over Na₂SO₄, filtered, and concentrated.

The crude residue was dissolved in DMF (3 mL) and ammonia (0.4 mL, 7N in MeOH), N,N-diisopropylethyamine (0.2 mL), and HATU (450 mg, 1.2 mmol) were added to the reaction mixture. The reaction was stirred at room temperature for 45 min. EtOAc and sat. aq. NaHCO₃ were added. The layers were separated and the organic phase was washed with brine, dried over Na₂SO₄, filtered, and concentrated. The crude residue was purified by silica gel chromatography (CHCl₃-ethanol) to give 4-bromo-5-cyclopropyl-1-(tetrahydro-2H-pyran-2-yl)-1H-indazole-6-carboxamide (Int-71A). MS (ESI) [M+H]⁺: m/z 364, 366.

### Step B: 4-bromo-5-cyclopropyl-1-(tetrahydro-2H-pyran-2-yl)-1H-indazole-6-carbonitrile (Int-71B)

Trifluoroacetic anhydride (0.21 mL, 1.5 mmol) was added to a suspension of 4-bromo-5-cyclopropyl-1-(tetrahydro-2H-pyran-2-yl)-1H-indazole-6-carboxamide (Int-71A) (137 mg, 0.376 mmol) and N,N-diisopropylethyamine (0.660 mL, 3.78 mmol) in CH₂Cl₂ (6 mL) at 0° C. The mixture was stirred at 0° C. for 15 min. EtOAc and sat. aq. NaHCO₃ were added. The layers were separated and the organic phase was washed with brine, dried over Na₂SO₄, filtered, and concentrated to give 4-bromo-5-cyclopropyl-1-(tetrahydro-2H-pyran-2-yl)-1H-indazole-6-carbonitrile (Int-71B). MS (ESI) [M+H]⁺: m/z 346, 348.

### Step C: (6-cyano-5-cyclopropyl-1-(tetrahydro-2H-pyran-2-yl)-1H-indazol-4-yl)boronic acid (Int-71)

A mixture of 4-bromo-5-cyclopropyl-1-(tetrahydro-2H-pyran-2-yl)-1H-indazole-6-carbonitrile (Int-71B) (120 mg, 0.346 mmol), tetrahydroxydiboron (155 mg, 1.73 mmol), cataCXium A Pd G3 (25 mg, 0.034 mmol), and Et₃N (0.34 ml, 2.44 mmol) in MeOH (2 ml) was stirred at room temperature for 2 h. After 2 h, EtOAc, CHCl₃ and H₃PO₄ (10% aq.) were added to the reaction. The layers were separated and the organic phase was washed with brine, dried over Na₂SO₄, filtered, and concentrated. The residue was purified by silica gel chromatography (CHCl₃-ethanol) to give (6-cyano-5-cyclopropyl-1-(tetrahydro-2H-pyran-2-yl)-1H-indazol-4-yl)boronic acid (Int-71). MS (ESI) [M+H]⁺: m/z 312.

### Intermediate 72:4-bromo-1-(tetrahydro-2H-pyran-2-yl)-1,6,7,8-tetrahydro-5H-benzo[f]indazol-5-one (Int-72)

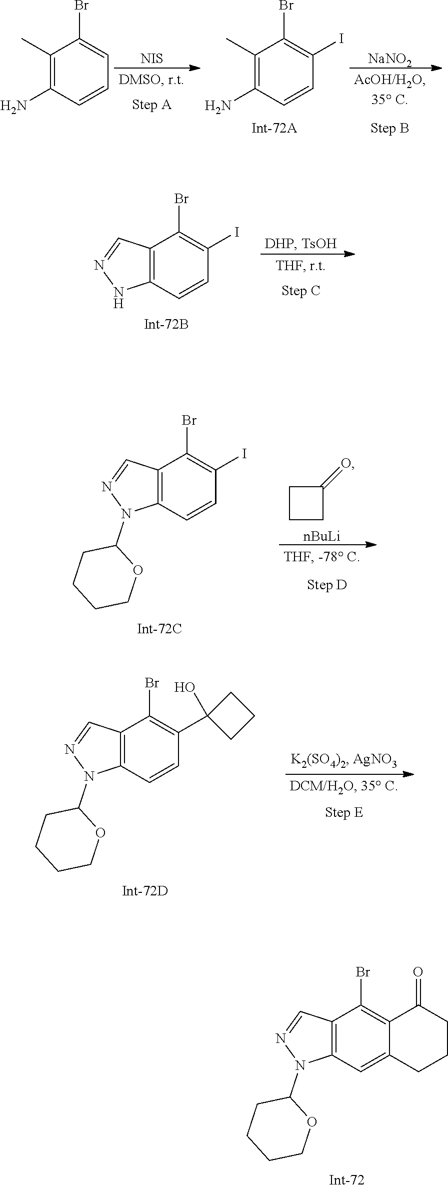

### Step A: 3-bromo-4-iodo-2-methylaniline (Int-72A)

Two reactions of the following were performed in parallel. 3-bromo-2-methylaniline (320 g, 1.72 mol) was dissolved in DMSO (2.00 L) and the solution was degassed with N₂. NIS (406 g, 1.81 mol) was added at room temperature and the reaction was stirred for 30 min. The reaction mixture was diluted with H₂O (15.0 L), filtered, washed with water, and the filter cake was dried under reduced pressure to obtain 3-bromo-4-iodo-2-methylaniline (Int-72A). ¹H NMR (400 MHz, CDCl₃) δ 7.48 (d, J=8.4 Hz, 1H), 6.42 (d, J=8.4 Hz, 1H), 2.39 (s, 3H).

### Step B: 4-bromo-5-iodo-1H-indazole (Int-72B)

Two reactions of the following were performed in parallel. 3-bromo-4-iodo-2-methylaniline (Int-72A) (375 g, 1.09 mol) was dissolved in AcOH (2.25 L) and degassed with N₂. A solution of NaNO₂ (90.3 g, 1.31 mol) in H₂O (225 mL) was added to the reaction mixture at room temperature. The reaction mixture was warmed to 35° C. and stirred for 44 hrs. The reaction mixture was diluted with H₂O (10.0 L), filtered, washed with water, and the filter cake was dried under reduced pressure to obtain 4-bromo-5-iodo-1H-indazole (Int-72B). ¹H NMR (400 MHz, CDCl₃) δ 8.04 (s, 1H), 7.77 (d, J=8.63 Hz, 1H), 7.25 (d, J=8.76 Hz, 1H).

### Step C: 4-bromo-5-iodo-1-(tetrahydro-2H-pyran-2-yl)-1H-indazole (Int-72C)

Three reactions of the following were performed in parallel. 4-bromo-5-iodo-1H-indazole (Int-72B) (320 g, 991 mmol) was dissolved in THF (2.00 L). The solution was degassed with N₂. Then 3,4-dihydro-2H-pyran (167 g, 1.98 mol) and p-toluenesulfonic acid (56.6 g, 297 mmol) were added at room temperature and the reaction was stirred for 6 h. The reaction was diluted with H₂O (1.00 L), extracted with EtOAc (2×1.00 L), and then washed with brine (500 mL). The combined organic layers were dried over Na₂SO₄, filtered, and concentrated. The crude residue was purified via silica gel chromatography (60:1 to 0:1 petroleum ether:EtOAc) to provide 4-bromo-5-iodo-1-(tetrahydro-2H-pyran-2-yl)-1H-indazole (Int-72C), which was used directly in the next step without further purification.

### Step D: 1-(4-bromo-1-(tetrahydro-2H-pyran-2-yl)-1H-indazol-5-yl)cyclobutan-1-ol (Int-72D)

Two reactions of the following were performed in parallel. 4-bromo-5-iodo-1-(tetrahydro-2H-pyran-2-yl)-1H-indazole (Int-72C) (210 g, 516 mmol) was dissolved in THF (2.40 L) and the solution was degassed with N₂. The reaction mixture was cooled to −78° C. Then cyclobutanone (181 g, 2.58 mol) was added at −78° C. followed by nBuLi (56.2 g, 877 mmol). The reaction mixture was stirred at −78° C. for 30 min. The reaction mixture was diluted the reaction mixture with sat. aq. NH₄Cl (2.00 L), extracted with EtOAc (3×2.00 L). The combined organic layers were dried over Na₂SO₄, filtered, and concentrated. The crude residue was purified by silica gel chromatography (petroleum ether:EtOAc=30:1 to 2:1) to provide 1-(4-bromo-1-(tetrahydro-2H-pyran-2-yl)-1H-indazol-5-yl)cyclobutan-1-ol (Int-72D). ¹H NMR (400 MHz, CDCl₃) δ 8.06 (s, 1H), 7.53 (d, J=8.58 Hz, 1H), 7.40 (d, J=8.58 Hz, 1H), 5.70 (dd, J=9.06 Hz, 2.74 Hz, 1H), 3.95-4.03 (m, 1H), 3.70-3.79 (m, 1H), 3.61 (m, 1H), 2.95-3.01 (m, 1H), 2.73-2.79 (m, 1H), 2.59-2.64 (m, 1H), 2.28-2.34 (m, 1H), 2.05-2.10 (m, 3H), 1.73-1.80 (m, 3H).

### Step E: 4-bromo-1-(tetrahydro-2H-pyran-2-yl)-1,6,7,8-tetrahydro-5H-benzo[f]indazol-5-one (Int-72)

Two reactions of the following were performed in parallel. 1-(4-bromo-1-(tetrahydro-2H-pyran-2-yl)-1H-indazol-5-yl)cyclobutan-1-ol (Int-72D) (100 g, 285 mmol) was dissolved in DCM (700 mL) and the solution was degassed with N₂. Then AgNO₃ (14.5 g, 85.1 mmol) and aqueous K₂(SO₄)₂ (385 g, 1.42 mol) were added at room temperature. The reaction was warmed to 35° C. and stirred for 16 hr. The reaction was cooled to room temperature and quenched with sat. aq. Na₂SO₃ (1.50 L). The reaction mixture was extracted with DCM (3×2.00 L) and the combined organic layers were dried over Na₂SO₄, filtered, and concentrated. The crude product was triturated with MTBE (200 mL) at 18° C. for 2 h to provide 4-bromo-1-(tetrahydro-2H-pyran-2-yl)-1,6,7,8-tetrahydro-5H-benzo[f]indazol-5-one (Int-72). MS (ESI): [M+H]⁺ m/z 349, 351. ¹H NMR (400 MHz, CDCl₃) δ 8.19 (s, 1H), 7.39 (s, 1H), 5.69 (m, 1H), 3.94-4.09 (m, 1H), 3.76 (m, 1H), 3.11 (m, 2H), 2.75 (t, J=6.63 Hz, 2H), 2.47-2.57 (m, 1H), 2.04-2.22 (m, 4H), 1.65-1.84 (m, 3H).

### Intermediate 73:4-bromo-1-(tetrahydro-2H-pyran-2-yl)-6,7-dihydrocyclopenta[f]indazol-5 (1H)-one (Int-73)

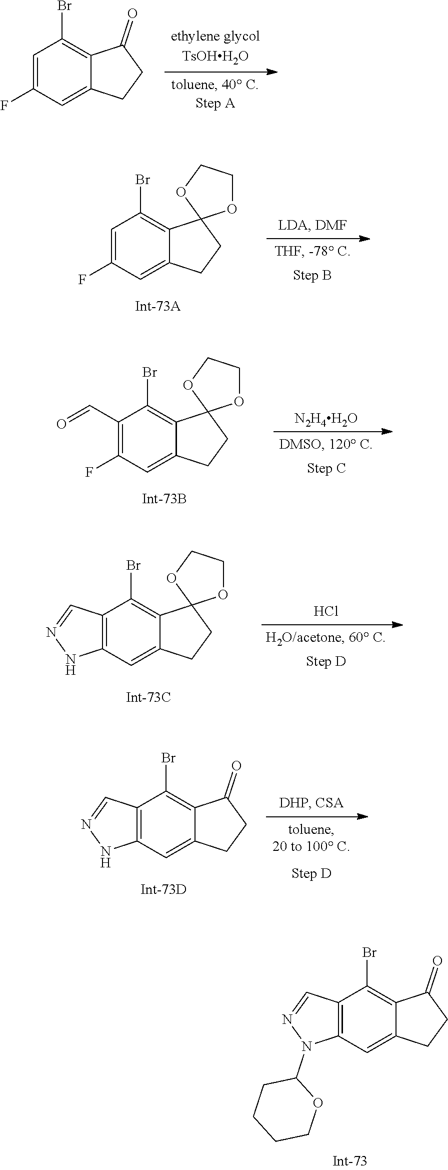

### Step A: 7-bromo-5-fluoro-2,3-dihydrospiro[indene-1,2′-[1,3]dioxolane](Int-73A)

Five reactions of the following were performed in parallel. 7-bromo-5-fluoro-2,3-dihydro-1H-inden-1-one (60.0 g, 262 mmol) was dissolved in toluene (2.58 L) at room temperature. Then, ethylene glycol (325 g, 5.24 mol) and TsOH·H₂O (9.97 g, 52.4 mmol) were added to the mixture at room temperature. A Dean-Stark trap was added to the reactor and the reaction was heated to 140° C. for 19 hr. The five reactions were cooled to room temperature, combined, and partitioned between EtOAc (3×2.50 L) and sat. aq. NaHCO₃ (3.00 L). The organic phase was washed with water (3×2.50 L) and brine, dried over Na₂SO₄, filtered, and concentrated. The crude residue was purified by silica gel chromatography (petroleum ether/EtOAc=50/1 to 1/1) to provide 7-bromo-5-fluoro-2,3-dihydrospiro[indene-1,2′-[1,3]dioxolane](Int-73A). ¹H NMR (400 MHz, CDCl₃) δ 7.18 (dd, J=8.4, 2.0 Hz, 1H), 6.89 (dd, J=8.4, 1.2 Hz, 1H), 4.32-4.36 (m, 2H), 4.09-4.11 (m, 2H), 2.90 (t, J=7.2 Hz, 2H), 2.32 (t, J=7.2 Hz, 2H).

### Step B: 7-bromo-5-fluoro-2,3-dihydrospiro[indene-1,2′-[1,3]dioxolane]-6-carbaldehyde (Int-73B)

Two reactions of the following were performed in parallel. 7-bromo-S-fluoro-2,3-dihydrospiro[indene-1,2′-[1,3]dioxolane](Int-73A) (80.0 g, 293 mmol) was dissolved in THF (1.60 L) at room temperature. The reaction was cooled to −78° C. and LDA (220 mL, 440 mmol, 2M in THF) was added. The reaction was stirred at −78° C. for 1 h. Then, DMF (85.7 g, 1.17 mol, 4.00 eq) was added to the mixture and the reaction was stirred at −78° C. for 0.5 h. The two reactions were combined and quenched with sat. aq. NH₄Cl (3.00 L), The reaction mixture was extracted with EtOAc (3×500 mL). The combined organic layers were washed with brine (1.00 L), dried over Na₂SO₄, filtered, and concentrated under reduced pressure to give a residue. The crude residue was triturated with MTBE (500 mL) for 30 min to provide 7-bromo-5-fluoro-2,3-dihydrospiro[indene-1,2′-[1,3]dioxolane]-6-carbaldehyde (Int-73B). ¹H NMR (400 MHz, CDCl₃) δ 10.43 (s, 1H), 7.01 (d, J=10.0 Hz, 1H), 4.34-4.36 (m, 2H), 4.10-4.14 (m, 2H), 2.95 (t, J=7.2 Hz, 2H), 2.34 (t, J=7.2 Hz, 2H).

### Step C: 4-bromo-6,7-dihydro-1H-spiro[cyclopenta[f]indazole-5,2′-[1,3]dioxolane](Int-73C)

Two reactions of the following were performed in parallel. 7-bromo-5-fluoro-2,3-dihydrospiro[indene-1,2′-[1,3]dioxolane]-6-carbaldehyde (Int-73B) (80.0 g, 266 mmol) was dissolved in DMSO (133 mL) and N₂H₄·H₂O (204 g, 3.99 mol) was added to the mixture at room temperature. The reaction was heated to 120° C. for 1.5 hrs. The two reaction mixtures were combined and quenched with water (1.0 L). The reaction mixture was extracted with EtOAc (2×500 mL) and the combined organic layers were washed with brine (500 mL), dried over Na₂SO₄, filtered, and concentrated under reduced pressure to give a residue. The crude residue was purify by silica gel chromatography (petroleum ether/EtOAc=50/1 to 0/1) to afford 4-bromo-6,7-dihydro-1H-spiro[cyclopenta[f]indazole-5,2′-[1,3]dioxolane](Int-73C). ¹H NMR (400 MHz, DMSO-de) δ 13.25 (s, 1H), 8.02 (s, 1H), 7.37 (s, 1H), 4.24 (t, J=3.6 Hz, 2H), 4.03 (t, J=3.6 Hz, 2H), 2.93 (t, J=6.4 Hz, 2H), 2.24 (t, J=7.2 Hz, 2H).

### Step D: 4-bromo-6,7-dihydrocyclopenta[f]indazol-5 (1H)-one (Int-73D)

4-bromo-6,7-dihydro-1H-spiro[cyclopenta[f]indazole-5,2′-[1,3]dioxolane](Int-73C) (100 g, 339 mmol) was dissolved in acetone (1.00 L) and H₂O (200 mL) at room temperature. Then, HCl (200 mL, 6M in H₂O) was added to the mixture at room temperature. The reaction was heated to 60° C. for 0.5 h. After 0.5 h, the reaction was cooled to room temperature and the pH was adjusted to ˜7-8 with sat. aq. NaHCO₃. The precipitated solid was collected by filtration, washed with water, and dried to afford 4-bromo-6,7-dihydrocyclopenta[f]indazol-5 (1H)-one (Int-73D). ¹H NMR (400 MHz, DMSO-d₆) δ 13.56 (s, 1H), 8.23 (s, 1H), 7.58 (s, 1H), 3.15 (t, J=6.8 Hz, 2H), 2.74 (t, J=6.8 Hz, 2H).

### Step E: 4-bromo-1-(tetrahydro-2H-pyran-2-yl)-6,7-dihydrocyclopenta[f]indazol-5 (1H)-one (Int-73)

4-bromo-6,7-dihydrocyclopenta[f]indazol-5 (1H)-one (Int-73D) (75.0 g, 299 mmol) was dissolved in toluene (1350 mL) at 20° C. and 3,4-dihydro-2H-pyran (100 g, 1.19 mol) and CSA (6.94 g, 29.9 mmol) was added to the mixture at room temperature. The reaction mixture was heated to 100° C. and stirred for 3 hrs. The reaction was quenched with sat. aq. NH₄Cl (2.0 L) and the organic layer was separated. The aqueous phase was extracted with DCM (2×0.5 L) and the combined organic layers were washed with brine (0.5 L), dried over sodium sulfate, filtered and concentrated under reduced pressure to give a residue. The crude residue was triturated with a mixture of MTBE and petroleum ether (3:2, 500 mL) at 25° C. for 30 mins to afford 4-bromo-1-(tetrahydro-2H-pyran-2-yl)-6,7-dihydrocyclopenta[f]indazol-5 (1H)-one (Int-73). ¹H NMR (400 MHz, CDCl₃) δ 8.20 (s, 1H), 7.53 (s, 1H), 5.72 (dd, J=9.2, 3.2 Hz, 1H), 4.00-4.03 (m, 1H), 3.76-3.77 (m, 1H), 3.20-3.23 (m, 2H), 2.81-2.85 (m, 2H), 2.49-2.53 (m, 1H), 2.08-2.18 (m, 2H), 1.70-1.78 (m, 3H).

### Intermediate 74: (1-(tetrahydro-2H-pyran-2-yl)-6,7-dihydro-1H-spiro[cyclopenta[f]indazole-5,1′-cyclopropan]-4-yl)boronic acid (Int-74)

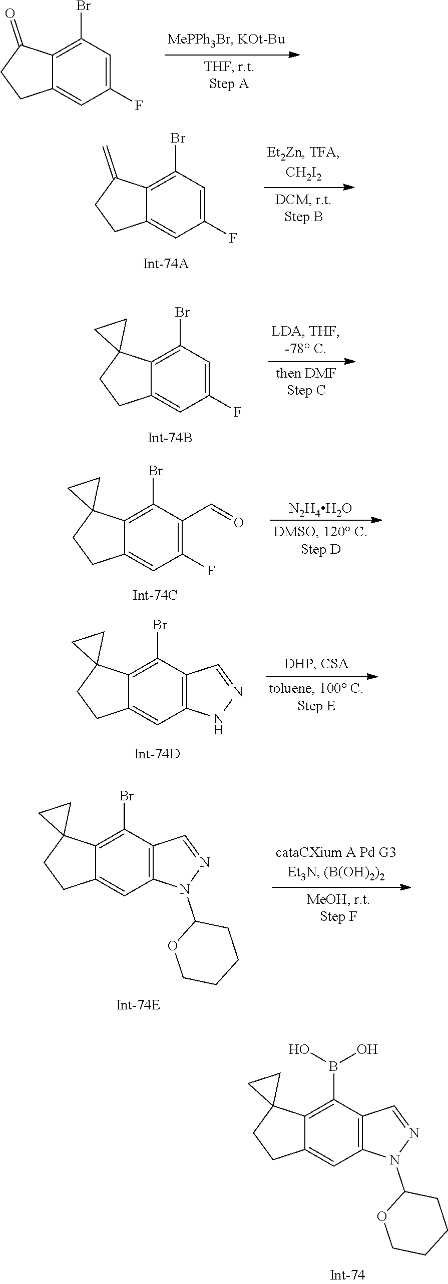

### Step A: 7-bromo-5-fluoro-1-methylene-2,3-dihydro-1H-indene (Int-74A)

To a stirred suspension of methyltriphenylphosphonium iodide (3.20 g, 7.90 mmol) in THF (50 mL) was added potassium tert-butoxide (0.820 g 7.30 mmol) in one portion. The yellowish suspension was stirred at room temperature for 1 h. Then 7-bromo-5-fluoro-indan-1-one (1.20 g, 5.20 mmol) was added to the mixture and stirred at room temperature for overnight. To the mixture was added sat. aq. NH₄Cl and EtOAc. The phases were shaken and separated and the organic phase was washed with brine, dried over Na₂SO₄, and concentrated. The residue was purified via silica gel chromatography (0 to 10% EtOAc in hexanes) to provide 7-bromo-5-fluoro-1-methylene-2,3-dihydro-1H-indene (Int-74A). ¹H NMR (400 MHz, CDCl₃) δ=7.20-7.12 (m, 1H), 6.94 (s, 1H), 6.16 (s, 1H), 5.24-5.16 (m, 1H), 3.01-2.92 (m, 2H), 2.90-2.82 (m, 2H).

### Step B: 7-bromo-5′-fluoro-2′,3′-dihydrospiro[cyclopropane-1,1′-indene](Int-74B)

To a stirred solution of diethylzinc (2.70 mL, 2.70 mmol, 1 M in hexanes) in DCM (3.2 mL) was added TFA (0.200 mL, 2.70 mmol) in DCM (1.50 mL) and stirred for 5 min. Then a solution of diiodomethane (0.220 ml, 2.70 mmol) in DCM (1.50 mL) was added to the mixture. After being stirred for 20 min, a solution of 7-bromo-5-fluoro-1-methylene-2,3-dihydro-1H-indene (Int-74A) (310 mg, 1.35 mmol) in DCM (1.50 mL) was added to the mixture and stirred for 1.5 h. To the mixture was added HCl (0.5N in H₂O) and stirred for 10 min. The mixture was extracted with EtOAc. The organic phase was washed with brine, dried over Na₂SO₄, and concentrated. The residue was purified via silica gel chromatography (0 to 10% EtOAc in hexanes) to provide 7′-bromo-5′-fluoro-2′,3′-dihydrospiro[cyclopropane-1,1′-indene](Int-74B). ¹H NMR (400 MHz, CDCl₃) δ=7.02-6.98 (m, 1H), 6.87 (tdd, J=1.1, 2.3, 8.1 Hz, 1H), 2.99 (dt, J=0.9, 7.8 Hz, 2H), 2.11 (t, J=7.8 Hz, 2H), 1.77-1.72 (m, 2H), 0.77-0.73 (m, 2H).

### Step C: 7′-bromo-5′-fluoro-2′,3′-dihydrospiro[cyclopropane-1,1′-indene]-6′-carbaldehyde (Int-74C)

To a stirred solution of 7′-bromo-5′-fluoro-2′,3′-dihydrospiro[cyclopropane-1,1′-indene](Int-74C) (310 mg, 1.29 mmol) in THF (13 mL) was added LDA (1.40 mL, 1.40 mmol, 1 M in THF) at −78° C. After being stirred at −78° C. for 0.5 h, DMF (0.20 mL, 2.57 mmol) was added to the mixture. After stirring for 0.5 h, the reaction was quenched with sat. aq. NH₄Cl and EtOAc. The layers were shaken and separated and the organic phase was washed with brine, dried over Na₂SO₄, and concentrated. The residue was purified via silica gel chromatography (10 to 50% EtOAc in hexanes) to give 7′-bromo-5′-fluoro-2′,3′-dihydrospiro[cyclopropane-1,1′-indene]-6′-carbaldehyde (Int-74C). ¹H NMR (400 MHz, CDCl₃) δ=10.36 (s, 1H), 6.98 (d, J=10.0 Hz, 1H), 3.07-3.01 (m, 2H), 2.18-2.10 (m, 2H), 1.95-1.89 (m, 2H), 0.85-0.77 (m, 2H).

### Step D: 4-bromo-6,7-dihydro-1H-spiro[cyclopenta[f]indazole-5,1′-cyclopropane](Int-74D)

To a stirred mixture of 7′-bromo-5′-fluoro-2′,3′-dihydrospiro[cyclopropane-1,1′-indene]-6′-carbaldehyde (Int-74C) (128 mg, 0.48 mmol) in DMSO (0.25 mL) was added hydrazine monohydrate (0.700 mL, 14.2 mmol) and then the mixture was stirred at 120° C. for 12 h. The mixture was cooled to room temperature and partitioned between water and EtOAc. The organic phase was washed with water (3×) and brine, dried over Na₂SO₄, and concentrated. The residue was purified via silica gel chromatography (5 to 60% EtOAc in hexanes) to give 4-bromo-6,7-dihydro-1H-spiro[cyclopenta[f]indazole-5,1′-cyclopropane](Int-74D). MS (ESI): m/z (M+H)⁺ 263.

### Step E: 4-bromo-1-(tetrahydro-2H-pyran-2-yl)-6,7-dihydro-1H-spiro[cyclopenta[f]indazole-5,1′-cyclopropane](Int-74E)

A mixture of 4-bromo-6,7-dihydro-1H-spiro[cyclopenta[f]indazole-5,1′-cyclopropane](Int-74D) (11.0 mg, 0.041 mmol), 3,4-dihydro-2H-pyran (0.008 mL, 0.083 mmol), (1R)-(−)-camphor-10-sulfonic acid (2.00 mg, 0.008 mmol) in toluene (0.5 mL) was stirred at 100° C. for 1 h. After cooling to room temperature, the mixture was partitioned between EtOAc and sat. aq. NaHCO₃. The organic phase was washed with brine, dried over Na₂SO₄, and concentrated. The residue was purified via silica gel chromatography (0 to 40% EtOAc in hexanes) to give 4-bromo-1-(tetrahydro-2H-pyran-2-yl)-6,7-dihydro-1H-spiro[cyclopenta[f]indazole-5,1′-cyclopropane](Int-74E). MS (ESI): m/z (M+H)⁺ 347.

### Step F: (1-(tetrahydro-2H-pyran-2-yl)-6,7-dihydro-1H-spiro[cyclopenta[f]indazole-5,1′-cyclopropan]-4-yl)boronic acid (Int-74)

To a stirred mixture of 4-bromo-1-(tetrahydro-2H-pyran-2-yl)-6,7-dihydro-1H-spiro[cyclopenta[f]indazole-5,1′-cyclopropane](Int-74E) (12.0 mg, 0.034 mmol), triethylamine (0.019 mL, 0.14 mmol) and tetrahydroxydiboron (6.00 mg, 0.069 mmol) in MeOH (0.7 mL) was added cataCXium A Pd G3 (3 mg, 0.003 mmol) and the mixture was stirred at room temperature for 3.5 h. The mixture was filtered through a pad of CELITE®, washed with MeOH, and the filtrate was concentrated. The residue was purified via silica gel chromatography (0 to 50% EtOAc in hexanes) to give (1-(tetrahydro-2H-pyran-2-yl)-6,7-dihydro-1H-spiro[cyclopenta[f]indazole-5,1′-cyclopropan]-4-yl)boronic acid (Int-74). MS (ESI): m/z (M+H)⁺ 313.

### Intermediate 75: (1-(tetrahydro-2H-pyran-2-yl)-4b,5,5a,6-tetrahydro-1H-cyclopropa[4,5]cyclopenta[1,2-f]indazol-4-yl)boronic acid (Int-75)

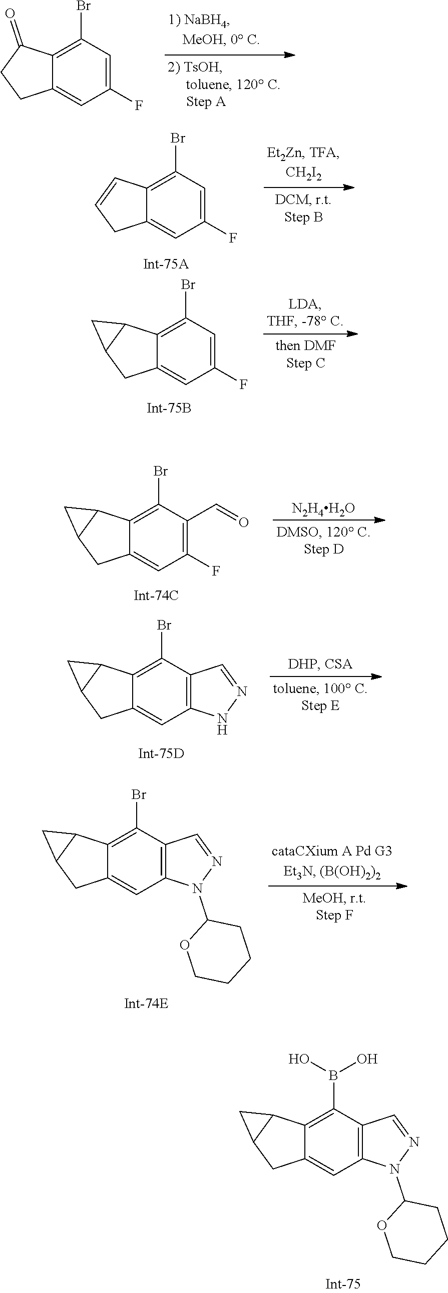

### Step A: 4-bromo-6-fluoro-1H-indene (Int-75A)

Sodium borohydride (496 mg, 13.1 mmol) was added to a stirred solution of 7-bromo-5-fluoro-indan-1-one (1.00 g, 4.36 mmol) in MeOH (40 mL) at 0° C. and the mixture was stirred for 1 h. The reaction was quenched with sat. aq. NH₄Cl and the mixture was concentrated in vacuo to remove MeOH. The resulting solution was extracted with EtOAc and the organic phase was washed with brine, dried over Na₂SO₄, filtered, and concentrated.

Toluene (18 mL) and p-toluenesulfonic acid (68.0 mg, 0.359 mmol) was added to the resulting crude residue and the mixture was stirred at 120° C. in a flask equipped with a Dean-Stark trap for 1 h. The reaction mixture was cooled to room temperature and concentrated. The crude residue was purified via silica gel chromatography (0 to 10% EtOAc in hexanes) to give 4-bromo-6-fluoro-1H-indene (Int-75A). ¹H NMR (400 MHz, CDCl₃) δ=7.20 (dd, J=2.2, 8.8 Hz, 1H), 7.18-7.13 (m, 1H), 6.96-6.91 (m, 1H), 6.61 (td, J=2.0, 5.6 Hz, 1H), 3.53-3.50 (m, 2H).

### Step B: 2-bromo-4-fluoro-1,1a,6,6a-tetrahydrocyclopropa[a]indene (Int-75B)

A solution of trifluoroacetic acid (0.781 mL, 6.85 mmol) in DCM (3.2 mL) was added to a stirred solution of diethylzinc (6.90 mL, 6.85 mmol, 1 M in hexane) in DCM (6.3 mL) and stirred for 20 min. Then, a solution of diiodomethane (0.553 mL, 6.85 mmol) in DCM (3.2 mL) was added to the mixture. After stirring for 20 min, a solution of 4-bromo-6-fluoro-1H-indene (Int-75A) (730 mg, 3.43 mmol) in DCM (3.2 mL) was added to the mixture and stirred for 1.5 h. HCl (0.5N in H₂O) was added to the reaction and stirred for 10 min. The mixture was extracted with EtOAc and the organic phase was washed with brine, dried over Na₂SO₄, filtered, and concentrated. The residue was purified via silica gel chromatography (0 to 10% EtOAc in hexanes) to give 2-bromo-4-fluoro-1,1a,6,6a-tetrahydrocyclopropa[a]indene (Int-75B). ¹H NMR (400 MHz, CDCl₃) δ=7.08-6.96 (m, 1H), 6.79 (td, J=0.9, 8.5 Hz, 1H), 3.27 (ddd, J=0.9, 6.8, 17.4 Hz, 1H), 3.03 (d, J=17.5 Hz, 1H), 2.49-2.43 (m, 1H), 1.94-1.83 (m, 1H), 1.14 (dt, J=4.6, 7.9 Hz, 1H), 0.14 (dt, J=3.4, 4.4 Hz, 1H).

### Step C: 2-bromo-4-fluoro-1,1a,6,6a-tetrahydrocyclopropa[a]indene-3-carbaldehyde (Int-75C)

LDA (3.60 mL, 3.60 mmol, 1 M in THF) was added to a stirred solution of 2-bromo-4-fluoro-1,1a,6,6a-tetrahydrocyclopropa[a]indene (Int-75B) (685 mg, 3.02 mmol) in THF (30 mL) at −78° C. The reaction was stirred at −78° C. for 0.5 h, then DMF (0.930 mL, 2.57 mmol) was added. After stirring for an additional 0.5 h at −78° C., the reaction was quenched with sat. aq. NH₄Cl. The reaction was extracted with EtOAc and the organic phase was washed with brine, dried over Na₂SO₄, filtered, and concentrated. The residue was purified via silica gel chromatography (0 to 20% EtOAc in hexanes) to give 2-bromo-4-fluoro-1,1a,6,6a-tetrahydrocyclopropa[a]indene-3-carbaldehyde (Int-75C). ¹H NMR (400 MHz, CDCl₃) δ=10.36-10.35 (m, 1H), 6.91 (d, J=10.3 Hz, 1H), 3.34 (dd, J=6.8, 18.4 Hz, 1H), 3.16-3.09 (m, 1H), 2.63-2.56 (m, 1H), 2.00-1.88 (m, 1H), 1.26-1.20 (m, 1H), 0.18 (dt, J=3.3, 4.6 Hz, 1H).

### Step D: 4-bromo-4b,5,5a,6-tetrahydro-1H-cyclopropa[4,5]cyclopenta[1,2-f]indazole (Int-75D)

Hydrazine monohydrate (2.40 mL, 48.5 mmol) was added to a stirred mixture of 2-bromo-4-fluoro-1,1a,6,6a-tetrahydrocyclopropa[a]indene-3-carbaldehyde (Int-75C) (412 mg, 1.62 mmol) in DMSO (0.80 mL) and the mixture was stirred at 120° C. for 12 h. The mixture was partitioned between water and EtOAc and the layers were separated. The organic phase was washed with water (3×) and brine, dried over Na₂SO₄, filtered, and concentrated. The residue was purified via silica gel chromatography (0 to 30% EtOAc in hexanes) to give 4-bromo-4b,5,5a,6-tetrahydro-1H-cyclopropa[4,5]cyclopenta[1,2-f]indazole (Int-75D). MS (ESI): m/z (M+H)⁺ 249.

### Step E: 4-bromo-1-(tetrahydro-2H-pyran-2-yl)-4b,5,5a,6-tetrahydro-1H-cyclopropa[4,5]cyclopenta[1,2-]indazole (Int-75E)

A mixture of 4-bromo-4b,5,5a,6-tetrahydro-1H-cyclopropa[4,5]cyclopenta[1,2-f]indazole (Int-75D) (138 mg, 0.554 mmol), 3,4-dihydro-2H-pyran (0.100 mL, 1.11 mmol), (1R)-(−)-camphor-10-sulfonic acid (25.7 mg, 0.11 mmol) in toluene (5 mL) was stirred at 100° C. for 1 h. After cooling to room temperature, the mixture was partitioned between EtOAc and sat. aq. NaHCO₃. The layers were separated and the organic phase was washed with brine, dried over Na₂SO₄, filtered, and concentrated. The residue was purified via silica gel chromatography (10 to 20% EtOAc in hexanes) to give 4-bromo-1-(tetrahydro-2H-pyran-2-yl)-4b,5,5a,6-tetrahydro-1H-cyclopropa[4,5]cyclopenta[1,2-f]indazole (Int-75E). MS (ESI): m/z (M+H)⁺ 332.

### Step F: (1-(tetrahydro-2H-pyran-2-yl)-4b,5,5a,6-tetrahydro-1H-cyclopropa[4,5]cyclopenta[1,2-f]indazol-4-yl)boronic acid (Int-75)

CataCXium A Pd G3 (20.5 mg, 0.028 mmol) was added to a stirred mixture of 4-bromo-1-(tetrahydro-2H-pyran-2-yl)-4b,5,5a,6-tetrahydro-1H-cyclopropa[4,5]cyclopenta[1,2-f]indazole (Int-75) (94.0 mg, 0.282 mmol), triethylamine (0.157 mL, 1.13 mmol), and tetrahydroxydiboron (50.6 mg, 0.564 mmol) in MeOH (5 mL) and the mixture was stirred at room temperature for 3.5 h. The mixture was filtered through a pad of CELITE®, washed with MeOH, and the filtrate was concentrated under reduced pressure. The residue was purified via silica gel chromatography (50 to 100% EtOAc in hexanes) to give (1-(tetrahydro-2H-pyran-2-yl)-4b,5,5a,6-tetrahydro-1H-cyclopropa[4,5]cyclopenta[1,2-f]indazol-4-yl)boronic acid (Int-75). MS (ESI): m/z (M+H)⁺ 299.

The compound in the table below was synthesized using a similar procedure as described in the synthesis of Int-75 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-76 | 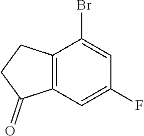 [SMILES: O=C1CCc2c(Br)cc(F)cc21] | 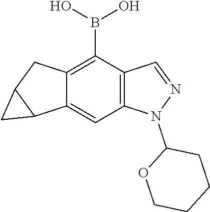 [SMILES: OB(O)c1c2c(cc3c1cnn3C1CCCCO1)C1CC1C2] | (1-(tetrahydro-2H- pyran-2-yl)-5,5,a,6,6a- tetrahydro-1H- cyclopropa[3,4] cyclopenta[1,2-f] indazol-4-yl)boronic acid | 299 |

### Intermediate 77: (E)-4-bromo-5-ethylidene-1-(tetrahydro-2H-pyran-2-yl)-5,6,7,8-tetrahydro-1H-benzo[f]indazole (Int-77)

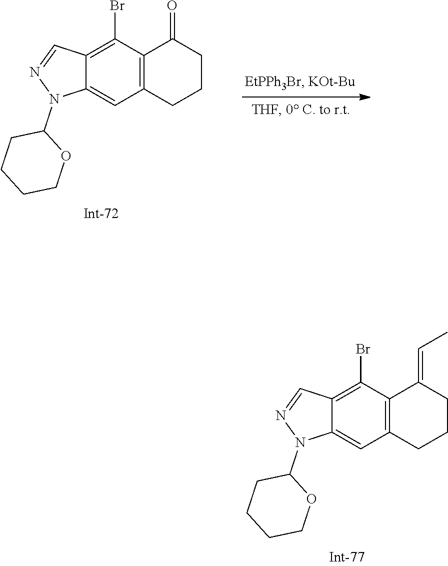 [SMILES: O=C1CCCc2cc3c(cnn3C3CCCCO3)c(Br)c21 ; C/C=C1\CCCc2cc3c(cnn3C3CCCCO3)c(Br)c21]

To a solution of ethyltriphenylphosphonium bromide (2.38 g, 6.40 mmol) in THF (10 mL) was added potassium tert-butoxide (6.40 mL, 6.40 mmol, 1 M in THF) at 25° C. The mixture was stirred at 25° C. for 1 h. 4-bromo-1-(tetrahydro-2H-pyran-2-yl)-1,6,7,8-tetrahydro-5H-benzo[f]indazol-5-one (Int-72) (1.12 g, 3.20 mmol) was dissolved in THF (10 mL) and added via syringe at 0° C. The mixture was warmed to room temperature and stir for 2 h. The mixture was quenched with sat. aq. NaHCO₃ (2 mL) and extracted with DCM (3×10 mL). The organic layer was dried over Na₂SO₄, filtered, and the filtrate was concentrated under reduced pressure. The residue was purified by flash silica gel chromatography (15% EtOAc in hexanes) to provide (E)-4-bromo-5-ethylidene-1-(tetrahydro-2H-pyran-2-yl)-5,6,7,8-tetrahydro-1H-benzo[f]indazole (Int-77). MS (ESI): [M+H]⁺ m/z 361, 363.

The compounds in the table below were synthesized using a similar procedure as described in the synthesis of Int-77 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-78 | 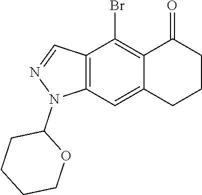 [SMILES: O=C1CCCc2cc3c(cnn3C3CCCCO3)c(Br)c21] Int-72 | 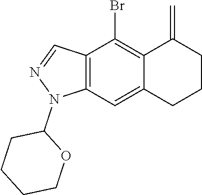 [SMILES: C=C1CCCc2cc3c(cnn3C3CCCCO3)c(Br)c21] | 4-bromo-5-methylene- 1-(tetrahydro-2H-pyran- 2-yl)-5,6,7,8- tetrahydro-1H- benzo[f]indazole | 347, 349 |
| Int-79 | 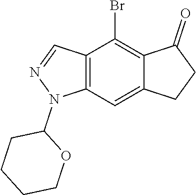 [SMILES: O=C1CCc2cc3c(cnn3C3CCCCO3)c(Br)c21] Int-73 | 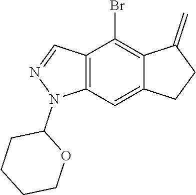 [SMILES: C=C1CCc2cc3c(cnn3C3CCCCO3)c(Br)c21] | 4-bromo-5-methylene- 1-(tetrahydro-2H-pyran- 2-yl)-1,5,6,7- tetrahydrocyclopenta[f] indazole | 333 |

### Intermediate 80: (5-methyl-1-(tetrahydro-2H-pyran-2-yl)-5,6,7,8-tetrahydro-1H-benzo[f]indazol-4-yl)boronic acid (Int-80)

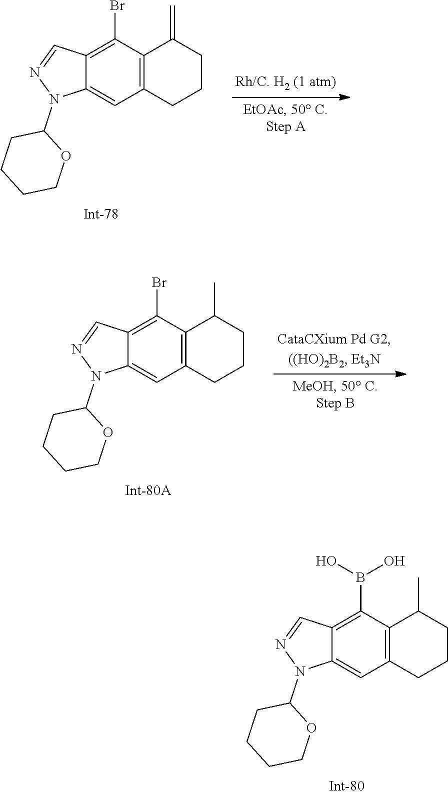 [SMILES: C=C1CCCc2cc3c(cnn3C3CCCCO3)c(Br)c21 ; CC1CCCc2cc3c(cnn3C3CCCCO3)c(Br)c21 ; CC1CCCc2cc3c(cnn3C3CCCCO3)c(B(O)O)c21]

### Step A: 4-bromo-5-methyl-1-(tetrahydro-2H-pyran-2-yl)-5,6,7,8-tetrahydro-1H-benzo[f]indazole (Int-80A)

A mixture of 4-bromo-5-methylene-1-(tetrahydro-2H-pyran-2-yl)-5,6,7,8-tetrahydro-1H-benzo[f]indazole (Int-78) (49.0 mg, 0.141 mmol), rhodium on carbon (145 mg, 5 wt. %) in EtOAc (2 mL) was degassed and charged with H₂ (3×) with a balloon. The mixture was heated to 50° C. and stirred for 1.5 h. After cooling to room temperature, the mixture was filtered through a pad of CELITE®, and the filtrate was concentrated to afford 4-bromo-5-methyl-1-(tetrahydro-2H-pyran-2-yl)-5,6,7,8-tetrahydro-1H-benzo[f]indazole (Int-80A). MS (ESI): [M+H]⁺ m/z 349, 351.

### Step B: (5-methyl-1-(tetrahydro-2H-pyran-2-yl)-5,6,7,8-tetrahydro-1H-benzo[f]indazol-4-yl)boronic acid (Int-80)

A vial was loaded with 4-bromo-5-methyl-1-(tetrahydro-2H-pyran-2-yl)-5,6,7,8-tetrahydro-1H-benzo[f]indazole (Int-80A) (277 mg, 0.793 mmol), hypodiboric acid (213 mg, 2.38 mmol), and cataCXium A Pd G2 (26.5 mg, 0.04 mmol). The reaction was dissolved in MeOH (2 mL) and triethylamine (0.332 mL, 2.38 mmol) under N₂. The mixture was stirred at 50° C. for 1 h under N₂ atmosphere. The mixture was concentrated under reduced pressure and purified via silica gel chromatography (0 to 100% EtOAc in hexanes) to provide (5-methyl-1-(tetrahydro-2H-pyran-2-yl)-5,6,7,8-tetrahydro-1H-benzo[f]indazol-4-yl)boronic acid (Int-80). MS (ESI): [M+H]⁺ m/z 315.

The compounds in the table below were synthesized using a similar procedure as described in the synthesis of Int-80 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-81 | 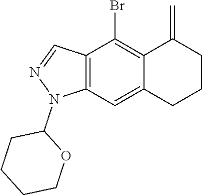 [SMILES: C=C1CCCc2cc3c(cnn3C3CCCCO3)c(Br)c21] Int-78 | 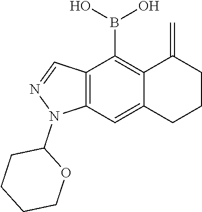 [SMILES: C=C1CCCc2cc3c(cnn3C3CCCCO3)c(B(O)O)c21] | (5-methylene-1- (tetrahydro-2H- pyran-2-yl)-5,6,7,8- tetrahydro-1H- benzo[f]indazol-4- yl)boronic acid | 313 |
| Int-82 | 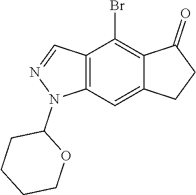 [SMILES: O=C1CCc2cc3c(cnn3C3CCCCO3)c(Br)c21] Int-73 | 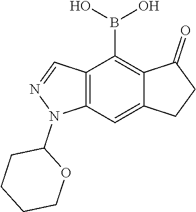 [SMILES: O=C1CCc2cc3c(cnn3C3CCCCO3)c(B(O)O)c21] | (5-oxo-1-(tetrahydro- 2H-pyran-2-yl)- 1,5,6,7- tetrahydrocyclopenta [f]indazol-4-yl) boronic acid | 301 |
| Int-83 | 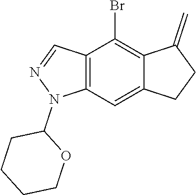 [SMILES: C=C1CCc2cc3c(cnn3C3CCCCO3)c(Br)c21] Int-79 | 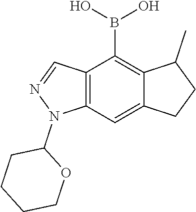 [SMILES: CC1CCc2cc3c(cnn3C3CCCCO3)c(B(O)O)c21] | (5-methyl-1- (tetrahydro-2H- pyran-2-yl)-1,5,6,7- tetrahydrocyclopenta [f]indazol-4-yl) boronic acid | 301 |

### Intermediate 84-1:4-bromo-1-(tetrahydro-2H-pyran-2-yl)-5-((triisopropylsilyl)ethynyl)-5,6,7,8-tetrahydro-1H-benzo[f]indazole (Int-84-1) & Intermediate 84-2:4-bromo-1-(tetrahydro-2H-pyran-2-yl)-5-((triisopropylsilyl)ethynyl)-7,8-dihydro-1-benzo[f]indazole (Int-84-2)

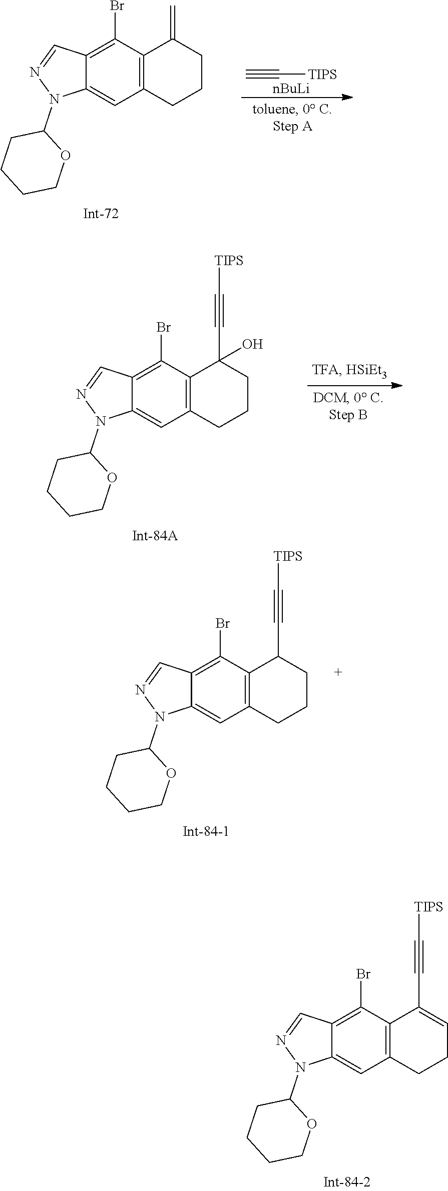

### Step A: 4-bromo-1-(tetrahydro-2H-pyran-2-yl)-5-((triisopropylsilyl)ethynyl)-5,6,7,8-tetrahydro-1H-benzo[f]indazol-5-ol (Int-84A)

Etbynyltriisopropylsilane (8.99 mL, 40.1 mmol) was dissolved in toluene (55 mL) under argon. Then, nBuLi (21.5 mL, 34.4 mmol, 1.6 M in Hexane) was added dropwise at room temperature and the mixture was stirred for 10 min. 4-bromo-1-(tetrahydro-2H-pyran-2-yl)-1,6,7,8-tetrahydro-5H-benzo[f]indazol-5-one (Int-72) (4.00 g, 11.5 mmol) was dissolved in toluene (10 mL) and added in one portion. The mixture was allowed to stir for 30 min at room temperature. The reaction was quenched with sat. aq. NH₄Cl (10 mL), extracted with DCM (3×30 mL), and purified via silica gel chromatography (0 to 100% EtOAc in hexanes) to afford 4-bromo-1-(tetrahydro-2H-pyran-2-yl)-5-((triisopropylsilyl)ethynyl)-5,6,7,8-tetrahydro-1H-benzo[f]indazol-5-ol (Int-84A). MS (ESI): m/z [M+H]⁺ 531, 533.

### Step B: 4-bromo-1-(tetrahydro-2H-pyran-2-yl)-5-((triisopropylsilyl)ethynyl)-5,6,7,8-tetrahydro-1H-benzo[f]indazole (Int-84-1) & 4-bromo-1-(tetrahydro-2H-pyran-2-yl)-5-((triisopropylsilyl)ethynyl)-7,8-dihydro-1H-benzo[f]indazole (Int-84-2)

4-bromo-1-(tetrahydro-2H-pyran-2-yl)-5-((triisopropylsilyl)ethynyl)-5,6,7,8-tetrahydro-1H-benzo[f]indazol-5-ol (Int-84A) (5.72 g, 10.8 mmol) was dissolved in DCM (50 mL) and cooled to 0° C. Triethylsilane (2.91 mL, 25.04 mmol) and TFA (3.29 mL, 43.0 mmol) were added via syringe. After 25 min of stirring at 0° C., the reaction was quenched with sat. aq. NaHCO₃ (50 mL), the layers were separated, and the aqueous phase was extracted with DCM (30 mL). The combined organic layers were evaporated under reduced pressure and the mixture was purified by silica gel chromatography (0 to 25% EtOAc in hexanes) to provide 4-bromo-1-(tetrahydro-2H-pyran-2-yl)-5-((triisopropylsilyl)ethynyl)-5,6,7,8-tetrahydro-1H-benzo[f]indazole (Int-84-1) [MS (ESI): [M+H]⁺ m/z 515, 517] and 4-bromo-1-(tetrahydro-2H-pyran-2-yl)-5-((triisopropylsilyl)ethynyl)-7,8-dihydro-1H-benzo[f]indazole (Int-84-2) [MS (ESI): [M+H]⁺ m/z 513, 515].

### Intermediate 85: (5S)-4-bromo-1-(tetrahydro-2H-pyran-2-yl)-5-((triisopropylsilyl)ethynyl)-5,6,7,8-tetrahydro-1H-benzo[f]indazole (Int-85)

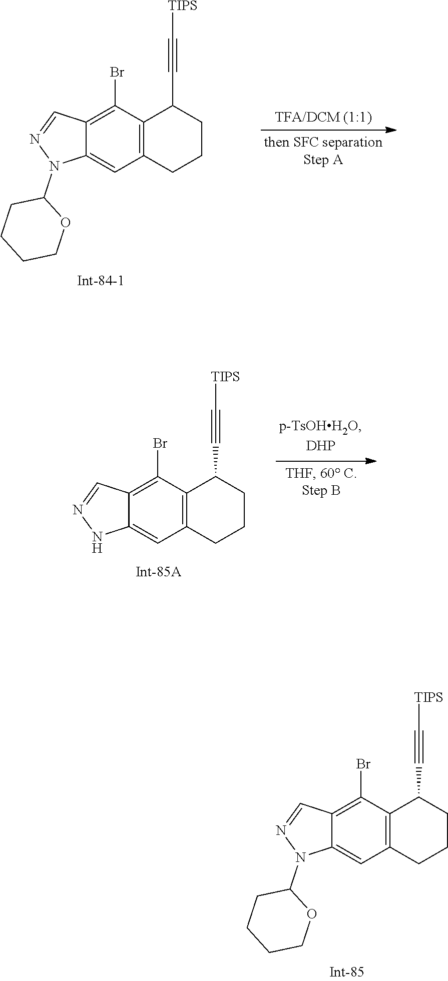

### Step A: (S)-4-bromo-5-((triisopropylsilyl)ethynyl)-5,6,7,8-tetrahydro-1H-benzo[f]indazole (Int-85A)

A vial was loaded with 4-bromo-1-(tetrahydro-2H-pyran-2-yl)-5-((triisopropylsilyl)ethynyl)-5,6,7,8-tetrahydro-1H-benzo[f]indazole (Int-84-1) (3.06 g, 5.93 mmol) and dissolved in TFA/DCM (12 mL, 1:1). The mixture was allowed to stir at room temperature for 90 min. The volatiles were evaporated under reduced pressure and the crude was redissolved in DCM (20 mL) and sat. aq. Na₂CO₃ (20 mL). The layers were separated and the aqueous layer was extracted with DCM (3×20 mL). The combined organic layers were dried over Na₂SO₄, filtered, and concentrated to afford racemic 4-bromo-5-((triisopropylsilyl)ethynyl)-5,6,7,8-tetrahydro-1H-benzo[f]indazole. The enantiomers were separated by chiral SFC (Column D, 30% MeOH w/0.1% NH₄OH) to afford(S)-4-bromo-5-((triisopropylsilyl)ethynyl)-5,6,7,8-tetrahydro-1H-benzo[f]indazole (Int-85A, first eluting isomer). MS (ESI): [M+H]⁺ m/z 431, 433.

### Step B: (5S)-4-bromo-1-(tetrahydro-2H-pyran-2-yl)-5-((triisopropylsilyl)ethynyl)-5,6,7,8-tetrahydro-1H-benzo[f]indazole (Int-85)

A vial was loaded with (S)-4-bromo-5-((triisopropylsilyl)ethynyl)-5,6,7,8-tetrahydro-1H-benzo[f]indazole (Int-85A) (198 mg, 0.459 mmol), p-toluenesulfonic acid monohydrate (26.2 mg, 0.138 mmol), and THF (1 mL). 3,4-dihydro-2H-pyran (84.0 μL, 0.918 mmol) was added and the mixture was allowed to stir at 60° C. for 16 h. The mixture was cooled to room temperature and quenched with sat. aq. NaHCO₃. The layers were separated and the aqueous layer was extracted with DCM (3×20 mL). The combined organic layers were dried with Na₂SO₄, filtered, and concentrated. The crude mixture was purified by silica gel chromatography (0 to 100% EtOAc in hexanes) to provide (5S)-4-bromo-1-(tetrahydro-2H-pyran-2-yl)-5-((triisopropylsilyl)ethynyl)-5,6,7,8-tetrahydro-1H-benzo[f]indazole (Int-85). MS (ESI): [M+H]⁺ m/z 515, 517.

### Intermediate 86: (5R)-4-bromo-5-ethyl-1-(tetrahydro-2H-pyran-2-yl)-5,6,7,8-tetrahydro-1H-benzo[f]indazole (Int-86)

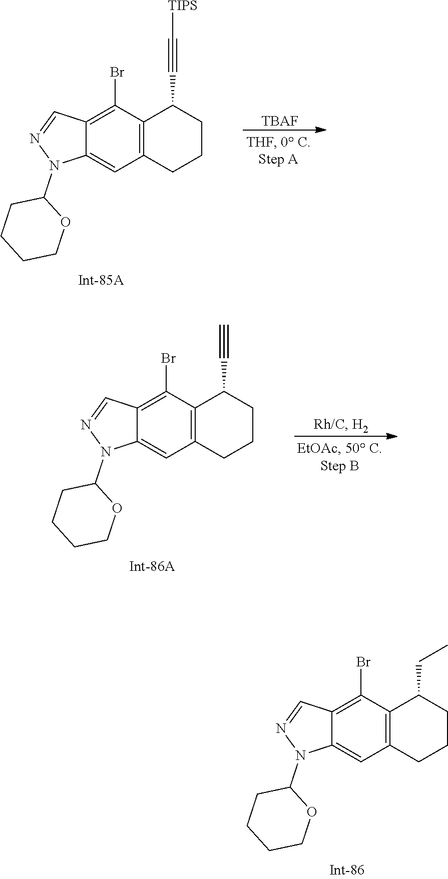

### Step A: (5S)-4-bromo-5-ethynyl-1-(tetrahydro-2H-pyran-2-yl)-5,6,7,8-tetrahydro-1H-benzo[f]indazole (Int-86A)

A vial was loaded with (5S)-4-bromo-1-(tetrahydro-2H-pyran-2-yl)-5-((triisopropylsilyl)ethynyl)-5,6,7,8-tetrahydro-1H-benzo[f]indazole (Int-85A) (500 mg, 1.16 mmol) and dissolved in THF (3 mL). TBAF (2.32 mL, 2.32 mmol, 1M in THF) was added slowly and stirred at room temperature for 2 h. The reaction was quenched with sat. aq. NH₄Cl and extracted with EtOAc (3×20 mL). The combined organic layers were washed with brine, dried over MgSO₄, filtered, and concentrated under reduced pressure to provide (5S)-4-bromo-5-ethynyl-1-(tetrahydro-2H-pyran-2-yl)-5,6,7,8-tetrahydro-1H-benzo[f]indazole (Int-86A) which was used directly in the next step without further purification. MS (ESI): [M+H]⁺ m/z 359, 361.

### Step B: (5R)-4-bromo-5-ethyl-1-(tetrahydro-2H-pyran-2-yl)-5,6,7,8-tetrahydro-1H-benzo[f]indazole (Int-86)

A vial was loaded with (5S)-4-bromo-5-ethynyl-1-(tetrahydro-2H-pyran-2-yl)-5,6,7,8-tetrahydro-1H-benzo[f]indazole (Int-86A) (416 mg, 1.16 mmol), Rh/C (477 mg, 0.232 mmol, 5 wt % Rh), and EtOAc (2 mL). H₂ gas was bubbled through a needle and a balloon and the mixture was heated to 50° C. under H₂ atmosphere for 16 h. The mixture was filtered through CELITE® and washed with EtOAc (20 mL). The mixture was concentrated and the residue was purified by silica gel chromatography (0 to 100% EtOAc in hexanes) to afford (5R)-4-bromo-5-ethyl-1-(tetrahydro-2H-pyran-2-yl)-5,6,7,8-tetrahydro-1H-benzo[f]indazole (Int-86). MS (ESI): [M+H]⁺ m/z 363, 365.

### Intermediate 87: (1-(tetrahydro-2H-pyran-2-yl)-5-(trifluoromethyl)-1,5,6,7-tetrahydrocyclopenta[f]indazol-4-yl)boronic acid (Int-87)

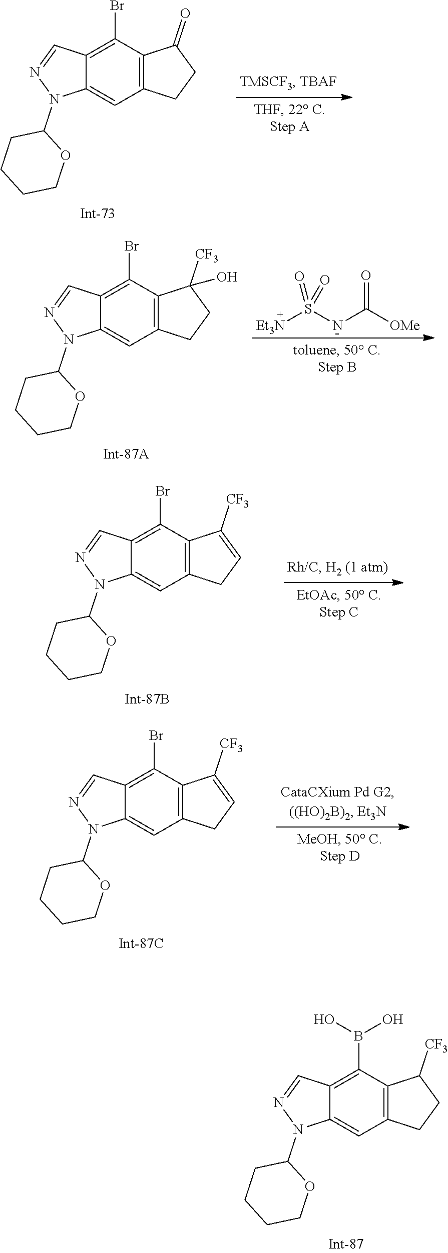

### Step A: 4-bromo-1-(tetrahydro-2H-pyran-2-yl)-5-(trifluoromethyl)-1,5,6,7-tetrahydrocyclopenta[f]indazol-5-ol (Int-87A)

A vial was loaded with 4-bromo-1-(tetrahydro-2H-pyran-2-yl)-6,7-dihydrocyclopenta[f]indazol-5 (1H)-one (Int-73) (662 mg, 1.98 mmol) and suspended in THF (5 mL). (Trifluoromethyl)trimethylsilane (0.497 mL, 3.36 mmol) was added followed by TBAF (40 μL, 0.040 mmol, 1 M in THF). The reaction was cooled to 0° C. and TBAF (1.70 mL, 1.7 mmol, 1 M in THF) was added. The mixture was allowed to stir at this temperature for 10 min and warmed to room temperature over 30 min. The mixture was concentrated under reduced pressure and the residue was purified by silica gel chromatography (0 to 100% EtOAc in hexanes) to provide 4-bromo-1-(tetrahydro-2H-pyran-2-yl)-5-(trifluoromethyl)-1,5,6,7-tetrahydrocyclopenta[f]indazol-5-ol (Int-87A). MS (ESI): [M+H]⁺ m/z 405, 407.

### Step B: 4-bromo-1-(tetrahydro-2H-pyran-2-yl)-5-(trifluoromethyl)-1,7-dihydrocyclopenta[f]indazole (Int-87B)

4-bromo-1-(tetrahydro-2H-pyran-2-yl)-5-(trifluoromethyl)-1,5,6,7-tetrahydrocyclopenta[f]indazol-5-ol (Int-87A) (440 mg, 1.846 mmol) and methyl N-(triethylammoniumsulfonyl) carbamate (450 mg, 1.111 mmol) were dissolved in toluene (5 mL). The reaction mixture heated to 50° C. and stirred for 1 h. After this time, additional methyl N-(triethylammoniumsulfonyl) carbamate (450 mg, 1.111 mmol) was added and the reaction was heated to 50° C. for 16 hr. The mixture was cooled to room temperature and concentrated under reduced pressure. The mixture was purified by silica gel chromatography (0 to 100% EtOAc in hexanes) to provide 4-bromo-1-(tetrahydro-2H-pyran-2-yl)-5-(trifluoromethyl)-1,7-dihydrocyclopenta[f]indazole (Int-87B). MS (ESI): [M+H]⁺ m/z 387, 389.

### Step C: 4-bromo-1-(tetrahydro-2H-pyran-2-yl)-5-(trifluoromethyl)-1,5,6,7-tetrahydrocyclopenta[f]indazole (Int-87C)

A vial was loaded with 4-bromo-1-(tetrahydro-2H-pyran-2-yl)-5-(trifluoromethyl)-1,7-dihydrocyclopenta[f]indazole (Int-87B) (79.2 mg, 0.205 mmol), rhodium on carbon (84.0 mg, 0.016 mmol, 5 wt %), and EtOAc (1.5 mL). H₂ gas was bubbled for 5 minutes with a balloon and the mixture was heated at 50° C. for 1 hour. The reaction mixture was filtered through a pad of CELITE® and washed with EtOAc. The mixture was concentrated under reduced pressure to obtain 4-bromo-1-(tetrahydro-2H-pyran-2-yl)-5-(trifluoromethyl)-1,5,6,7-tetrahydrocyclopenta[f]indazole (Int-87C), which was used directly in the next step without further purification. MS (ESI): [M+H]⁺ m/z 389, 391.

### Step D: (1-(tetrahydro-2H-pyran-2-yl)-5-(trifluoromethyl)-1,5,6,7-tetrahydrocyclopenta[f]indazol-4-yl)boronic acid (Int-87)

A vial was loaded with 4-bromo-1-(tetrahydro-2H-pyran-2-yl)-5-(trifluoromethyl)-1,5,6,7-tetrahydrocyclopenta[f]indazole (Int-87C) (71.6 mg, 0.184 mmol), hypodiboric acid (49.5 mg, 0.552 mmol), and cataCXium A Pd G2 (6.1 mg, 9.20 μmol). The reaction was dissolved in MeOH (1 mL) and triethylamine (77.0 μL, 0.552 mmol) under N₂. The mixture was stirred at 50° C. for 1 h under N₂ atmosphere. The mixture was concentrated in vacuo, redissolved in EtOAc, and washed with sat. aq. NH₄Cl. The layers were shaken and separated and the aqueous phase was extracted with EtOAc. The combined organic layers were washed with brine, dried over Na₂SO₄, filtered, and evaporated under reduced pressure to provide (1-(tetrahydro-2H-pyran-2-yl)-5-(trifluoromethyl)-1,5,6,7-tetrahydrocyclopenta[f]indazol-4-yl)boronic acid (Int-87), which was used directly in the next step without further purification. MS (ESI): [M+H]⁺ m/z 355.

### Intermediate 88: (1-(tetrahydro-2H-pyran-2-yl)-5-vinyl-1,5,6,7-tetrahydrocyclopenta[f]indazol-4-yl)boronic acid (Int-88)

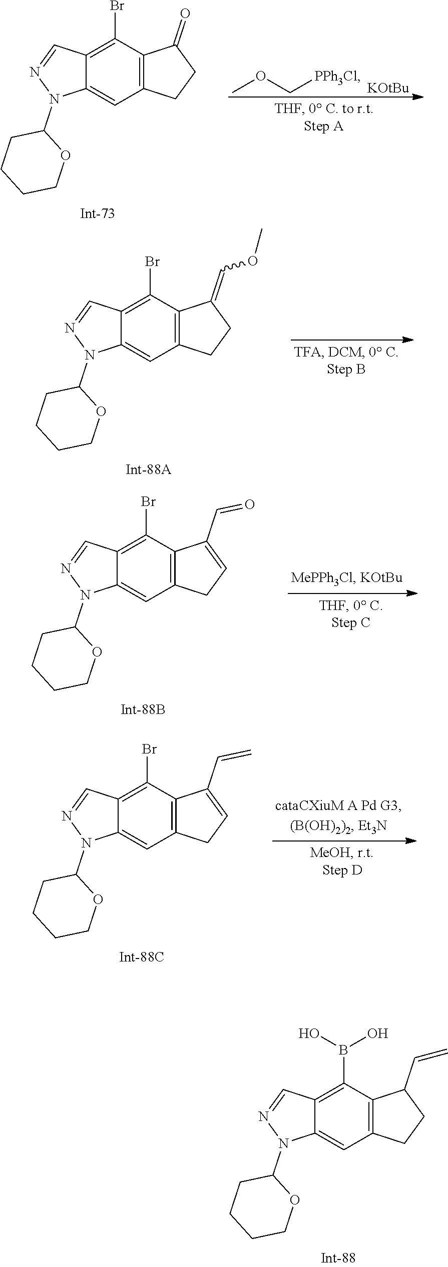

### Step A: 4-bromo-5-(methoxymethylene)-1-(tetrahydro-2H-pyran-2-yl)-1,5,6,7-tetrahydrocyclopenta[f]indazole (Int-88A)

To a stirred suspension of (methoxymethyl)triphenylphosphonium chloride (4.10 g, 12.0 mmol) in THF (40 mL) was added potassium tert-butoxide (12.0 mL, 12.0 mmol, 1 M in THF) at 0° C. The resulting suspension was stirred at room temperature for 0.5 h then 4-bromo-1-(tetrahydro-2H-pyran-2-yl)-6,7-dihydrocyclopenta[f]indazol-5 (1H)-one (Int-73) (2.00 g, 6.00 mmol) in THF (120 mL) was added to the mixture. The reaction mixture was stirred at room temperature for 1 h. To the mixture was added sat. aq. NH₄Cl and EtOAc. The layers were separated and the organic phase was washed with brine, dried over Na₂SO₄, filtered, and concentrated. The crude residue was purified via silica gel chromatography (0 to 40% EtOAc in hexanes) to provide 4-bromo-5-(methoxymethylene)-1-(tetrahydro-2H-pyran-2-yl)-1,5,6,7-tetrahydrocyclopenta[f]indazole (Int-88A). MS (ESI): m/z (M+H)⁺ 363, 365.

### Step B: 4-bromo-1-(tetrahydro-2H-pyran-2-yl)-1,5,6,7-tetrahydrocyclopenta[f]indazole-5-carbaldehyde (Int-88B)

To a stirred solution of 4-bromo-5-(methoxymethylene)-1-(tetrahydro-2H-pyran-2-yl)-1,5,6,7-tetrahydrocyclopenta[f]indazole (Int-88A) (1.68 g, 4.6 mmol) in DCM (7.5 mL) was added TFA (7.5 mL) at 0° C. After stirring for 1 h, the reaction was diluted with CHCl₃ (30 mL) and 5 N NaOH (25 mL) was added gradually to basify the reaction mixture. The layers were separated and organic phase was washed with brine, dried over Na₂SO₄, filtered, and concentrated. The crude residue was purified via silica gel chromatography (10 to 75% EtOAc in hexanes) to give 4-bromo-1-(tetrahydro-2H-pyran-2-yl)-1,5,6,7-tetrahydrocyclopenta[f]indazole-5-carbaldehyde (Int-88B). MS (ESI): m/z (M+H)⁺ 349, 351.

### Step C: 4-bromo-1-(tetrahydro-2H-pyran-2-yl)-5-vinyl-1,5,6,7-tetrahydrocyclopenta[f]indazole (Int-88C)

To a stirred suspension of methyltriphenylphosphonium iodide (717 mg, 1.78 mmol) in THF (10 mL) was added potassium tert-butoxide (199 mg, 1.78 mmol) in one portion and the mixture was stirred at room temperature for 0.5 h. Then 4-bromo-1-(tetrahydro-2H-pyran-2-yl)-1,5,6,7-tetrahydrocyclopenta[f]indazole-5-carbaldehyde (Int-88B) (310 mg, 0.890 mmol) in THF (2 mL) was added to the mixture and stirred at room temperature for 1 h. Sat. aq. NH₄Cl and EtOAc was added and the layers were shaken and separated. The organic phase was washed with brine, dried over dried over Na₂SO₄, filtered, and concentrated. The crude residue was purified via silica gel chromatography (0 to 30% EtOAc in hexanes) to give 4-bromo-1-(tetrahydro-2H-pyran-2-yl)-5-vinyl-1,5,6,7-tetrahydrocyclopenta[f]indazole (Int-88C). MS (ESI): m/z (M+H)⁺ 347, 349.

### Step D: (1-(tetrahydro-2H-pyran-2-yl)-5-vinyl-1,5,6,7-tetrahydrocyclopenta[f]indazol-4-yl)boronic acid (Int-88)

To a stirred mixture of 4-bromo-1-(tetrahydro-2H-pyran-2-yl)-5-vinyl-1,5,6,7-tetrahydrocyclopenta[f]indazole (Int-88C) (47.0 mg, 0.140 mmol), triethylamine (0.075 mL, 0.541 mmol), and tetrahydroxydiboron (24 mg, 0.271 mmol) in MeOH (2.7 mL) was added cataCXium A Pd G3 (5 mg, 0.007 mmol) and the mixture was stirred at room temperature for 1.5 h. The mixture was filtered through a pad of CELITE® and washed with MeOH. The filtrate was concentrated under reduced pressure and the residue was purified via silica gel chromatography (40 to 100% EtOAc in hexanes) to afford (1-(tetrahydro-2H-pyran-2-yl)-5-vinyl-1,5,6,7-tetrahydrocyclopenta[f]indazol-4-yl) boronic acid (Int-88). MS (ESI): m/z (M+H)⁺ 313.

### Intermediate 89: (5-(methoxymethyl)-1-(tetrahydro-2H-pyran-2-yl)-1,5,6,7-tetrahydrocyclopenta[f]indazol-4-yl)boronic acid (Int-89)

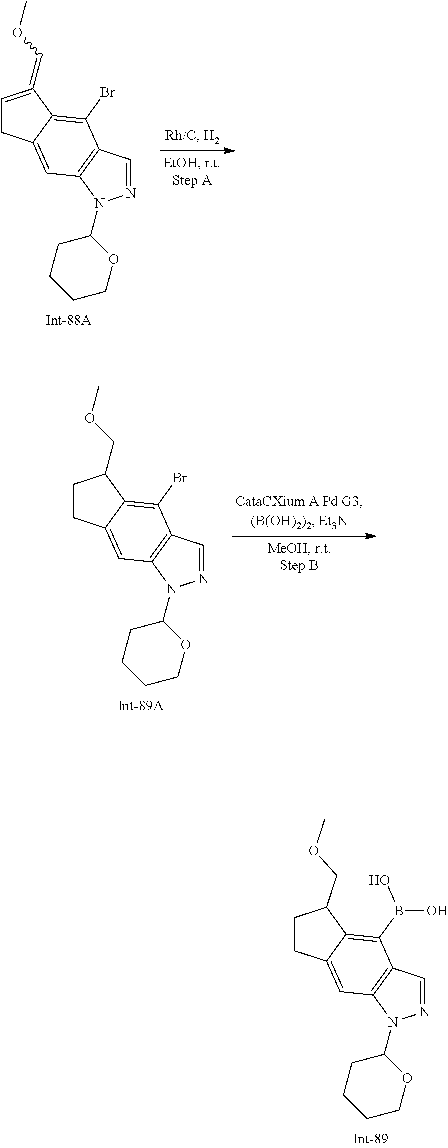

### Step A: 4-bromo-5-(methoxymethyl)-1-(tetrahydro-2H-pyran-2-yl)-1,5,6,7-tetrahydrocyclopenta[f]indazole (Int-89A)

Rh/C (240 mg, 5% w/w) was added to a solution of 4-bromo-5-(methoxymethylene)-1-(tetrahydro-2H-pyran-2-yl)-1,5,6,7-tetrahydrocyclopenta[f]indazole (Int-88A) (80 mg, 0.22 mmol) in EtOH (4.4 mL). The reaction was fitted with a H₂ balloon and allowed to stir at room temperature overnight. The reaction mixture was filtered through a pad of celite and the filtrate was concentrated under reduced pressure. The residue was purified by silica gel chromatography (0 to 100% hexane/EtOAc) to afford 4-bromo-5-(methoxymethyl)-1-(tetrahydro-2H-pyran-2-yl)-1,5,6,7-tetrahydrocyclopenta[f]indazole (Int-89A). MS (ESI) [M+H]⁺: m/z 365, 367.

### Step B: (5-(methoxymethyl)-1-(tetrahydro-2H-pyran-2-yl)-1,5,6,7-tetrahydrocyclopenta[f]indazol-4-yl)boronic acid (Int-89)

A mixture of 4-bromo-5-(methoxymethyl)-1-(tetrahydro-2H-pyran-2-yl)-1,5,6,7-tetrahydrocyclopenta[f]indazole (Int-89A), tetrahydroxydiboron (58.9 mg, 0.660 mmol), cataCXium A Pd G3 (80.0 mg, 0.11 mmol), and Et₃N (0.121 mL, 0.880 mmol) in MeOH (4.4 mL) was stirred at room temperature for 1 h. The reaction mixture was filtered through a pad of CELITE®, washed with MeOH, and the filtrate was concentrated under reduced pressure. The residue was purified by silica gel chromatography (0 to 10% CHCl₃/MeOH) to afford (5-(methoxymethyl)-1-(tetrahydro-2H-pyran-2-yl)-1,5,6,7-tetrahydrocyclopenta[f]indazol-4-yl)boronic acid (Int-89). MS (ESI) [M+H]⁺: 331.

### Intermediate 90: (1-(tetrahydro-2H-pyran-2-yl)-5-((triisopropylsilyl)ethynyl)-1,5,6,7-tetrahydrocyclopenta[f]indazol-4-yl)boronic acid (Int-90)

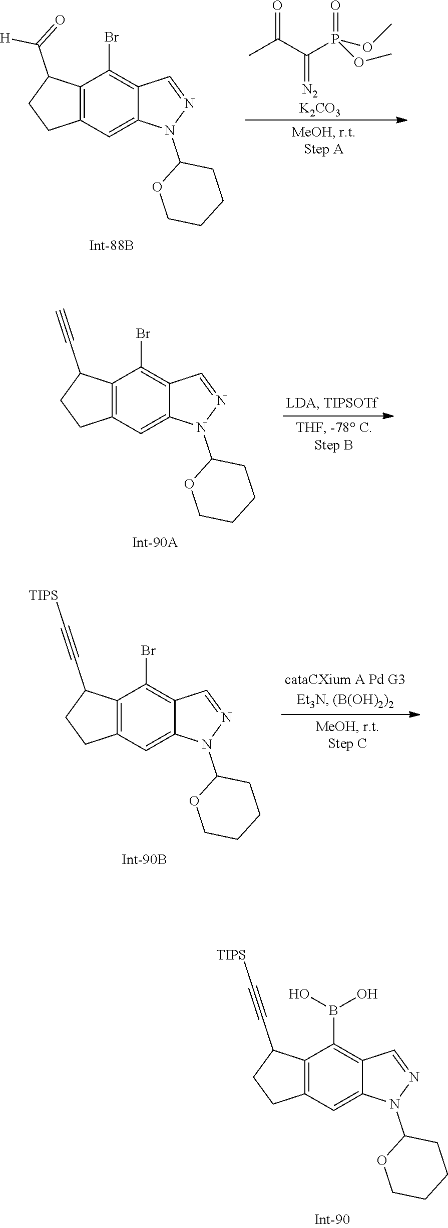

### Step A: 4-bromo-5-ethynyl-1-(tetrahydro-2H-pyran-2-yl)-1,5,6,7-tetrahydrocyclopenta[f]indazole (Int-90A)

To a stirred solution of 4-bromo-1-(tetrahydro-2H-pyran-2-yl)-1,5,6,7-tetrahydrocyclopenta[f]indazole-5-carbaldehyde (Int-88B) (133 mg, 0.381 mmol) and K₂CO₃ (105 mg, 0.762 mmol) in MeOH (4 ml) was added dimethyl(1-diazo-2-oxopropyl)phosphonate (0.137 mL, 0.571 mmol). The mixture was stirred at room temperature for 1 h. To the mixture was added sat. aq. NH₄Cl and EtOAc. The layers were shaken and separated and the organic phase was washed with brine, dried over Na₂SO₄, and concentrated. The residue was purified via silica gel chromatography (10 to 40% EtOAc in hexanes) to give 4-bromo-5-ethynyl-1-(tetrahydro-2H-pyran-2-yl)-1,5,6,7-tetrahydrocyclopenta[f]indazole (Int-90A). MS (ESI): m/z (M+H)⁺ 345, 347.

### Step B: 4-bromo-1-(tetrahydro-2H-pyran-2-yl)-5-((triisopropylsilyl)ethynyl)-1,5,6,7-tetrahydrocyclopenta[f]indazole (Int-90B)

To a stirred solution of 4-bromo-5-ethynyl-1-(tetrahydro-2H-pyran-2-yl)-1,5,6,7-tetrahydrocyclopenta[f]indazole (Int-90A) (92.0 mg, 0.266 mmol) in THF (5 mL) was added LDA (0.400 mL, 0.400 mmol, 1 M in THF) at −78° C. After stirring for 0.5 h, triisopropylsilyl trifluoromethanesulfonate (0.110 mL, 0.40 mmol) was added and the mixture was warmed to 0° C. After 1 h, the mixture was partitioned between EtOAc and water. The organic phase was washed with brine, dried over Na₂SO₄, and concentrated. The residue was purified via silica gel chromatography (0 to 30% EtOAc in hexanes) to give 4-bromo-1-(tetrahydro-2H-pyran-2-yl)-5-((triisopropylsilyl)ethynyl)-1,5,6,7-tetrahydrocyclopenta[f]indazole (Int-90B), m/z (M+H)⁺ 501, 503.

### Step C: (1-(tetrahydro-2H-pyran-2-yl)-5-(trisopropylsilyl)ethynyl)-1,5,6,7-tetrahydrocyclopenta[f]indazol-4-yl)boronic acid (Int-90)

To a stirred mixture of 4-bromo-1-(tetrahydro-2H-pyran-2-yl)-5-((triisopropylsilyl)ethynyl)-1,5,6,7-tetrahydrocyclopenta[f]indazole (Int-90B) (87.0 mg, 0.173 mmol), triethylamine (0.145 mL, 1.04 mmol), and tetrahydrodiboron (78.0 mg, 0.867 mmol) in MeOH (1.7 mL) was added cataCXium A Pd G3 (13.0 mg, 0.017 mmol) and the mixture was stirred at room temperature for 3.5 h. The mixture was filtered through a pad of CELITE®, washed with MeOH, and the filtrate was concentrated. The residue was purified via silica gel chromatography (20 to 75% EtOAc in hexanes) to give (1-(tetrahydro-2H-pyran-2-yl)-5-((triisopropylsilyl)ethynyl)-1,5,6,7-tetrahydrocyclopenta[f]indazol-4-yl)boronic acid (Int-90). MS (ESI): m/z (M+H)⁺ 467.

### Intermediate 91: (5-ethyl-1-(tetrahydro-2H-pyran-2-yl)-1,5,6,7-tetrahydrocyclopenta[f]indazol-4-yl)boronic acid (Int-91)

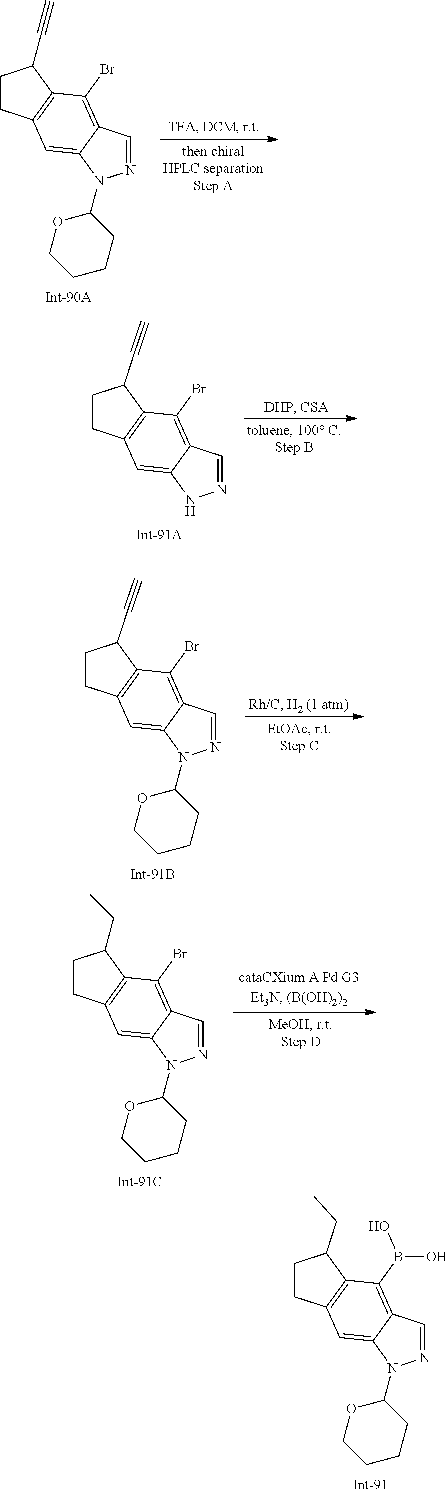

### Step A: 4-bromo-5-ethynyl-1,5,6,7-tetrahydrocyclopenta[f]indazole (Int-91A)

TFA (1.50 mL, 20.0 mmol) was added to a stirred solution of 4-bromo-5-ethynyl-1-(tetrahydro-2H-pyran-2-yl)-1,5,6,7-tetrahydrocyclopenta[f]indazole (Int-90A) (310 mg, 0.898 mmol) in DCM (1.50 mL) at room temperature. After stirring for 1 h, the mixture was basified by NaOH (5N in H₂O) and the mixture was extracted with EtOAc. The organic phase was washed with brine, dried over Na₂SO₄, filtered, and concentrated. The crude residue was purified via silica gel chromatography (20 to 60% EtOAc in hexane). The racemic mixture was separated by Chiral HPLC (ChiralPak AD-H (eluant Hex/EtOH=85/15)) to give 4-bromo-5-ethynyl-1,5,6,7-tetrahydrocyclopenta[f]indazole (Int-91A, first eluting isomer). MS (ESI): m/z (M+H)⁺ 261, 263.

### Step B: 4-bromo-5-ethynyl-1-(tetrahydro-2H-pyran-2-yl)-1,5,6,7-tetrahydrocyclopenta[f]indazole (Int-91B)

A mixture of 4-bromo-5-ethynyl-1,5,6,7-tetrahydrocyclopenta[f]indazole (Int-91A) (69.0 mg, 0.264 mmol), 3,4-dihydro-2H-pyran (0.048 mL, 0.528 mmol), and (1R)-(−)-camphor-10-sulfonic acid (6.1 mg, 0.026 mmol) in toluene (2.5 mL) was stirred at 100° C. for 3 h. The reaction was cooled to room temperature and partitioned between EtOAc and sat. aq. NaHCO₃. The layers were separated and the organic phase was washed with brine, dried over Na₂SO₄, filtered, and concentrated. The crude residue was purified via silica gel chromatography (0 to 30% EtOAc in hexane) to afford 4-bromo-5-ethynyl-1-(tetrahydro-2H-pyran-2-yl)-1,5,6,7-tetrahydrocyclopenta[f]indazole (Int-91B). MS (ESI): m/z (M+H)⁺ 345.

### Step C: 4-bromo-5-ethyl-1-(tetrahydro-2H-pyran-2-yl)-1,5,6,7-tetrahydrocyclopenta[f]indazole (Int-91C)

A mixture of 4-bromo-5-ethynyl-1-(tetrahydro-2H-pyran-2-yl)-1,5,6,7-tetrahydrocyclopenta[f]indazole (Int-91B) (57.0 mg, 0.165 mmol) and rhodium on carbon (114 mg, 5 wt %) in EtOAc (5.70 mL) was evacuated and backfilled with H₂. After stirring at room temperature for 1.5 h, the mixture was filtered through a pad of CELITE®, washed with EtOAc, and the filtrate was concentrated to give 4-bromo-5-ethyl-1-(tetrahydro-2H-pyran-2-yl)-1,5,6,7-tetrahydrocyclopenta[f]indazole (Int-91C), which was used directly in the next step without purification. MS (ESI): m/z (M+H)⁺ 349, 351.

### Step D: (5-ethyl-1-(tetrahydro-2H-pyran-2-yl)-1,5,6,7-tetrahydrocyclopenta[f]indazol-4-yl)boronic acid (Int-91)

A mixture of 4-bromo-5-ethyl-1-(tetrahydro-2H-pyran-2-yl)-1,5,6,7-tetrahydrocyclopenta[f]indazole (Int-91C) (44.6 mg, 0.128 mmol), tetrahydroxydiboron (34.3 mg, 0.383 mmol), triethylamine (71.2 μL, 0.511 mmol), and cataCXium A Pd G3 (9.30 mg, 0.0128 mmol) in MeOH (2.55 mL) was stirred at room temperature for 3 h. After 3 h, the reaction was filtered through a pad of celite and washed with MeOH. The filtrate was concentrated and the residue was purified via silica gel chromatography (10 to 100% EtOAc in hexane) to give (5-ethyl-1-(tetrahydro-2H-pyran-2-yl)-1,5,6,7-tetrahydrocyclopenta[f]indazol-4-yl)boronic acid (Int-91). MS (ESI): m/z (M+H)⁺ 315.

### Intermediate 92: (5-propyl-1-(tetrahydro-2H-pyran-2-yl)-1,5,6,7-tetrahydrocyclopenta[f]indazol-4-yl)boronic acid (Int-92)

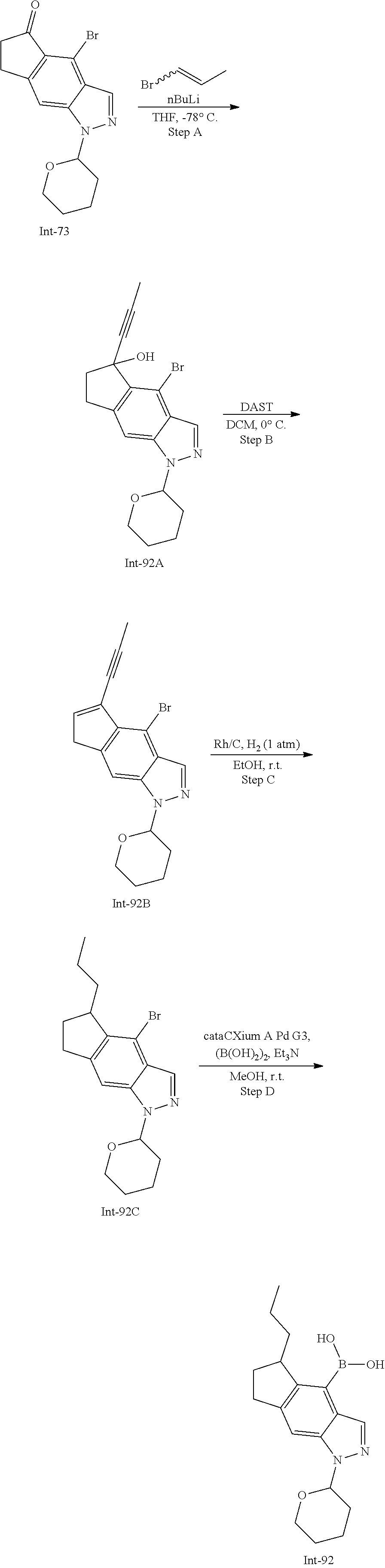

### Step A: 4-bromo-5-(prop-1-yn-1-yl)-1-(tetrahydro-2H-pyran-2-yl)-1,5,6,7-tetrahydrocyclopenta[f]indazol-5-ol (Int-92A)

A mixture of 1-bromoprop-1-ene (38.2 μL, 0.447 mmol) and nBuLi (0.432 mL, 0.656 mmol, 1.5 M in hexane) in THF (0.5 mL) was stirred at −78° C. for 1 h. A solution of 4-bromo-1-(tetrahydro-2H-pyran-2-yl)-6,7-dihydrocyclopenta[f]indazol-5 (1H)-one (Int-73) (100 mg, 0.298 mmol) in THF (5.00 mL) was added via syringe and the reaction was stirred at −78° C. for 1.5 b. Then, an additional mixture of 1-bromoprop-1-ene (115 μL, 1.34 mmol) and nBuLi (0.864 mL, 1.31 mmol, 1.52 M in hexane) in THF (1.00 mL) was added via syringe and the reaction was stirred at −78° C. for 3 h. After 3 hours, the reaction was diluted with EtOAc and sat. aq. NH₄Cl. The layers were separated and the aqueous phase was extracted with EtOAc. The combined organic layers were washed with brine, dried over Na₂SO₄, filtered, and concentrated. The crude residue was purified via silica gel chromatography (10 to 90% EtOAc in hexane) to provide 4-bromo-5-(prop-1-yn-1-yl)-1-(tetrahydro-2H-pyran-2-yl)-1,5,6,7-tetrahydrocyclopenta[f]indazol-5-ol (Int-92A). MS (ESI): m/z (M+H)⁺ 375, 377.

### Step B: 4-bromo-5-(prop-1-yn-1-yl)-1-(tetrahydro-2H-pyran-2-yl)-1,7-dihydrocyclopenta[f]indazole (Int-92B)

A mixture of 4-bromo-5-(prop-1-yn-1-yl)-1-(tetrahydro-2H-pyran-2-yl)-1,5,6,7-tetrahydrocyclopenta[f]indazol-5-ol (Int-92A) (78.7 mg, 0.210 mmol), DAST (83.1 μL, 0.629 mmol) in CH₂Cl₂ (3.94 mL) was stirred at 0° C. for 3 min. The reaction was diluted with EtOAc and sat. aq. NaHCO₃. The layers were shaken and separated and the aqueous phase was extracted with EtOAc. The combined organic layers were washed with brine, dried over Na₂SO₄, filtered, and concentrated. The crude residue was purified via silica gel chromatography (0 to 40% EtOAc in hexane) to give 4-bromo-5-(prop-1-yn-1-yl)-1-(tetrahydro-2H-pyran-2-yl)-1,7-dihydrocyclopenta[f]indazole (Int-92B). MS (ESI): m/z (M+H)⁺ 357, 359.

### Step C: 4-bromo-5-propyl-1-(tetrahydro-2H-pyran-2-yl)-1,5,6,7-tetrahydrocyclopenta[f]indazole (Int-92C)

A mixture of 4-bromo-5-(prop-1-yn-1-yl)-1-(tetrahydro-2H-pyran-2-yl)-1,7-dihydrocyclopenta[f]indazole (Int-92B) (14.6 mg, 0.0409 mmol) and rhodium on carbon (29.2 mg, 5 wt %) in EtOH (3.00 mL) was evacuated and back-filled with H₂. After stirring at room temperature for 1.5 h, the mixture was filtered through a pad of CELITE® and washed with EtOH. The filtrate was concentrated in vacuo to provide 4-bromo-5-propyl-1-(tetrahydro-2H-pyran-2-yl)-1,5,6,7-tetrahydrocyclopenta[f]indazole (Int-92C). MS (ESI): m/z (M+H)⁺ 363, 365.

### Step D: (5-propyl-1-(tetrahydro-2H-pyran-2-yl)-1,5,6,7-tetrahydrocyclopenta[f]indazol-4-yl)boronic acid (Int-92)

A mixture of 4-bromo-5-propyl-1-(tetrahydro-2H-pyran-2-yl)-1,5,6,7-tetrahydrocyclopenta[f]indazole (Int-92C) (11.0 mg, 0.0303 mmol), tetrahydroxydiboron (8.14 mg, 0.0908 mmol), triethylamine (16.9 μL, 0.121 mmol), and cataCXium A Pd G3 (2.21 mg, 0.303 μmol) in MeOH (0.606 mL) was stirred at room temperature for 3 h. The reaction was filtered through a pad of CELITE® and washed with MeOH. The filtrate was concentrated in vacuo and the crude residue was purified via silica gel chromatography (10 to 100% EtOAc in hexane, then 0 to 20% MeOH in EtOAc) to provide (5-propyl-1-(tetrahydro-2H-pyran-2-yl)-1,5,6,7-tetrahydrocyclopenta[f]indazol-4-yl)boronic acid (Int-92). MS (ESI): m/z (M+H)⁺ 329.

### Intermediate 93: (5-cyclopropyl-1-(tetrahydro-2H-pyran-2-yl)-1,7-dihydrocyclopenta[f]indazol-4-yl)boronic acid (Int-93)

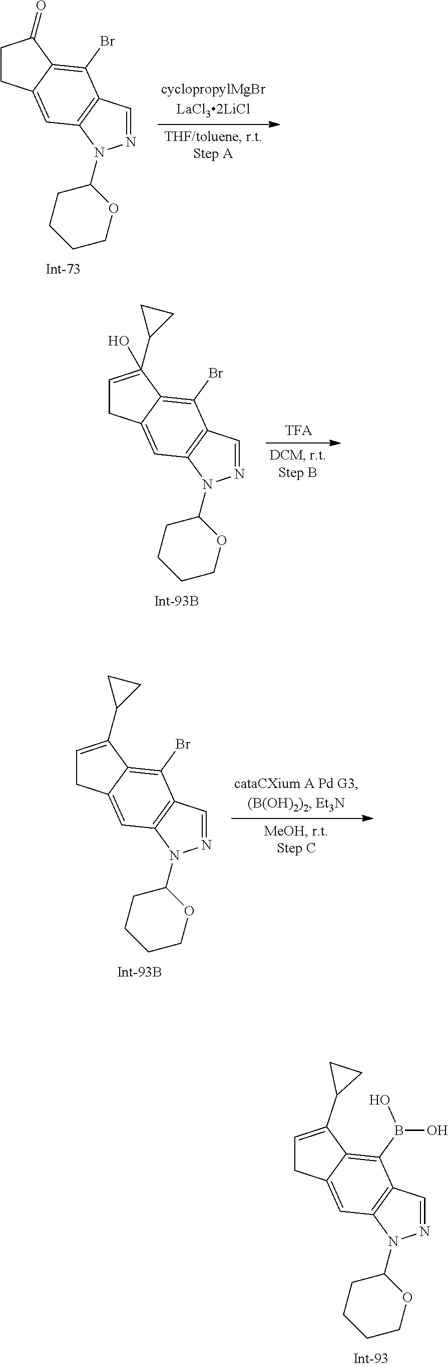

### Step A: 4-bromo-5-cyclopropyl-1-(tetrahydro-2H-pyran-2-yl)-1,5,6,7-tetrahydrocyclopenta[f]indazol-5-ol (Int-93A)

Cyclopropylmagnesium bromide (10.0 mL, 7.46 mmol, 0.7 M in THF) was added to a mixture of 4-bromo-1-(tetrahydro-2H-pyran-2-yl)-6,7-dihydrocyclopenta[f]indazol-5 (1H)-one (Int-73) (500 mg, 1.49 mmol) and LaCl₃·2LiCl (0.5 mL, 0.298 mmol, 0.6 M in THF) in THF (7.5 mL) and toluene (7.5 mL) at room temperature. After stirring for 30 min, the reaction mixture was diluted with EtOAc and sat. aq. NH₄Cl. The layers were separated and the organic phase was washed with brine. The organic phase was dried over Na₂SO₄, filtered, and concentrated under reduced pressure to afford 4-bromo-5-cyclopropyl-1-(tetrahydro-2H-pyran-2-yl)-1,5,6,7-tetrahydrocyclopenta[f]indazol-5-ol (Int-93A), which was used directly in the next step without further purification. MS (ESI) [M+H]⁺: m/z 377, 379.

### Step B: 4-bromo-5-cyclopropyl-1-(tetrahydro-2H-pyran-2-yl)-1,7-dihydrocyclopenta[f]indazole (Int-93B)

A solution of 4-bromo-5-cyclopropyl-1-(tetrahydro-2H-pyran-2-yl)-1,5,6,7-tetrahydrocyclopenta[f]indazol-5-ol (562 mg, 1.49 mmol) (Int-93A) in DCM (7.5 mL) and TFA (0.02 mL) was stirred at room temperature for 0.5 h. The mixture was concentrated under reduced pressure, and the residue was purified by column chromatography on silica gel (0 to 100% hexane/EtOAc) to afford 4-bromo-5-cyclopropyl-1-(tetrahydro-2H-pyran-2-yl)-1,7-dihydrocyclopenta[f]indazole (Int-93B). MS (ESI) [M+H]⁺: m/z 359, 361.

### Step C: (5-cyclopropyl-1-(tetrahydro-2H-pyran-2-yl)-1,7-dihydrocyclopenta[f]indazol-4-yl)boronic acid (Int-93)

Tetrahydroxydiboron (29.9 mg, 0.02 mmol), triethylamine (0.062 mL, 0.45 mmol), and cataXCium A Pd G3 (8.10 mg, 0.011 mmol) were added to a solution of 4-bromo-5-cyclopropyl-1-(tetrahydro-2H-pyran-2-yl)-1,7-dihydrocyclopenta[f]indazole (Int-93B) (40.0 mg, 0.09 mmol) in MeOH (2.2 mL). The mixture was stirred at room temperature for 2 h under N₂. The mixture was concentrated under reduced pressure and the residue was purified by flash silica gel chromatography (0 to 100% EtOAc in hexane) to afford (5-cyclopropyl-1-(tetrahydro-2H-pyran-2-yl)-1,7-dihydrocyclopenta[f]indazol-4-yl) boronic acid (Int-93). MS (ESI) [M+H]⁺: m/z 325.

### Intermediate 94: (5-cyclopropyl-1-(tetrahydro-2H-pyran-2-yl)-1,5,6,7-tetrahydrocyclopenta[f]indazol-4-yl)boronic acid (Int-94)

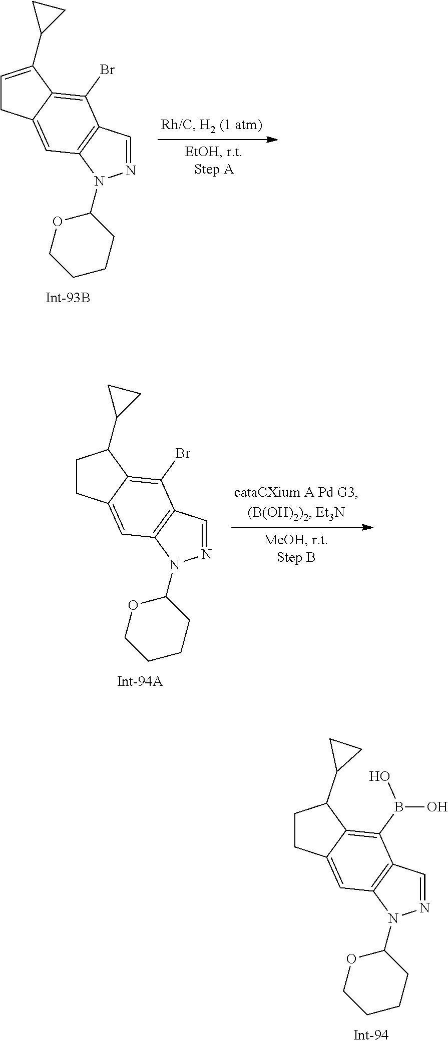 [SMILES: Brc1c2c(cc3c1cnn3C1CCCCO1)CC=C2C1CC1 ; Brc1c2c(cc3c1cnn3C1CCCCO1)CCC2C1CC1 ; OB(O)c1c2c(cc3c1cnn3C1CCCCO1)CCC2C1CC1]

### Step A: 4-bromo-5-cyclopropyl-1-(tetrahydro-2H-pyran-2-yl)-1,5,6,7-tetrahydrocyclopenta[f]indazole (Int-94A)

Rh/C (400 mg, 5 wt %) was added to a solution of 4-bromo-5-cyclopropyl-1-(tetrahydro-2H-pyran-2-yl)-1,7-dihydrocyclopenta[f]indazole (Int-93B) (0.20 g, 0.567 mmol) in EtOH (40 mL). The reaction vessel was evacuated and backfilled with H₂. After stirring at room temperature under H₂ atmosphere for 2 h, the mixture was filtered through a pad of CELITE® and the filtrate was concentrated under reduced pressure. The crude residue was purified by silica gel chromatography (0 to 100% EtOAc in hexane) affording 4-bromo-5-cyclopropyl-1-(tetrahydro-2H-pyran-2-yl)-1,5,6,7-tetrahydrocyclopenta[f]indazole (Int-94A). MS (ESI) [M+H]⁺: m/z 361, 363.

### Step B: (5-cyclopropyl-1-(tetrahydro-2H-pyran-2-yl)-1,5,6,7-tetrahydrocyclopenta[f]indazol-4-yl)boronic acid (Int-94)

Tetrahydroxydiboron (24.5 mg, 0.02 mmol), triethyamine (0.05 mL, 0.36 mmol), and cataCXium A Pd G3 (6.6 mg, 0.009 mmol) were added to a solution of 4-bromo-5-cyclopropyl-1-(tetrahydro-2H-pyran-2-yl)-1,5,6,7-tetrahydrocyclopenta[f]indazole (Int-94A) (33.0 mg, 0.09 mmol) in MeOH (1.8 mL). The mixture was stirred at room temperature for 1 h under N₂ atmosphere. The reaction mixture was concentrated under reduced pressure and the resulting crude residue was purified by flash silica gel chromatography (hexane/EtOAc=95/5 to 0/100) to afford (5-cyclopropyl-1-(tetrahydro-2H-pyran-2-yl)-1,5,6,7-tetrahydrocyclopenta[f]indazol-4-yl)boronic acid (Int-94). MS (ESI) [M+H]⁺: m/z 327.

### Intermediate 95: (1-(tetrahydro-2H-pyran-2-yl)-1,5,6,7-tetrahydrocyclopenta[f]indazol-4-yl)boronic acid (Int-95)

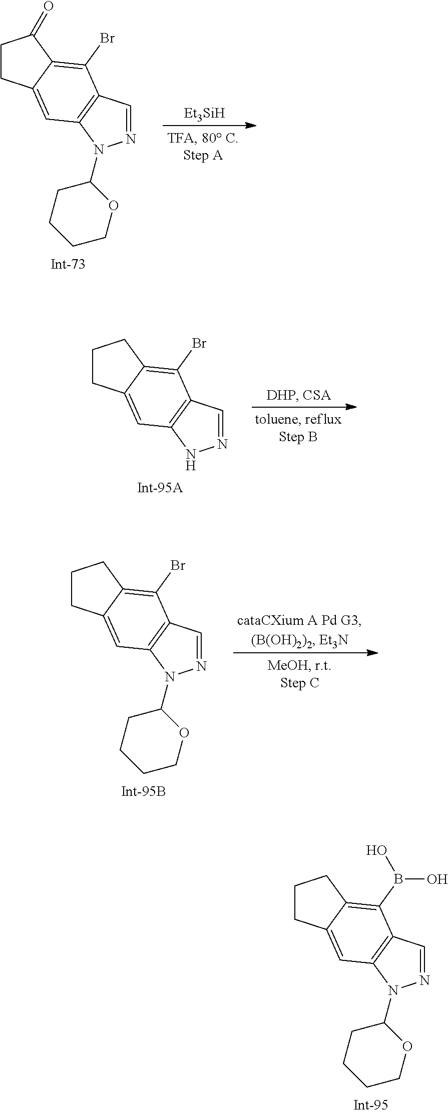

### Step A: 4-bromo-1,5,6,7-tetrahydrocyclopenta[f]indazole (Int-95A)

Triethylsilane (1.91 mL, 11.9 mmol) was added to a solution of 4-bromo-1-(tetrahydro-2H-pyran-2-yl)-6,7-dihydrocyclopenta[f]indazol-5 (1H)-one (Int-73) (400 mg, 1.19 mmol) in TFA (6.0 mL) at room temperature. The reaction mixture was warmed to 80° C. and stirred for 2 h. The reaction mixture was cooled to room temperature and quenched with sat. aq. NaHCO₃ and diluted with CHCl₃. The organic layer was separated, dried over Na₂SO₄, filtered, and concentrated in vacuo to afford 4-bromo-1,5,6,7-tetrahydrocyclopenta[f]indazole (Int-95A), which was used directly in the next step without further purification. MS (ESI) [M+H]⁺: m/z 237, 239.

### Step B: 4-bromo-1-(tetrahydro-2H-pyran-2-yl)-1,5,6,7-tetrahydrocyclopenta[f]indazole (Int-95B)

A mixture of 4-bromo-1,5,6,7-tetrahydrocyclopenta[f]indazole (282 mg, 1.19 mmol) (Int-95A), (1R)-(−)-camphor-10-sulfonic acid (55.0 mg, 0.239 mmol), and 3,4-dihydro-2H-pyran (0.43 mL, 4.77 mmol) in toluene (12 mL) was stirred at 120° C. for 2 h. The mixture was cooled to room temperature and concentrated under reduced pressure. The residue was purified by column chromatography (hexane/EtOAc=100/0 to 20/80) to afford 4-bromo-1-(tetrahydro-2H-pyran-2-yl)-1,5,6,7-tetrahydrocyclopenta[f]indazole (Int-95B). MS (ESI) [M+H]⁺: m/z 321, 323.

### Step C: (1-(tetrahydro-2H-pyran-2-yl)-1,5,6,7-tetrahydrocyclopenta[f]indazol-4-yl)boronic acid (Int-95)

A mixture of 4-bromo-1-(tetrahydro-2H-pyran-2-yl)-1,5,6,7-tetrahydrocyclopenta[f]indazole (Int-95B) (38.0 mg, 0.133 mmol), tetrahydroxydiboron (126 mg, 1.40 mmol), cataCXium A Pd G3 (34.0 mg, 0.047 mmol), and Et₃N (0.260 mL, 1.87 mmol) in MeOH (4.7 ml) was stirred at room temperature for 1 h. The reaction mixture was filtered through a pad of CELITE®, washed with MeOH, and the filtrate was concentrated under reduced pressure. The residue was purified by silica gel chromatography (0 to 20% MeOH in CHCl₃) to afford (1-(tetrahydro-2H-pyran-2-yl)-1,5,6,7-tetrahydrocyclopenta[f]indazol-4-yl)boronic acid (Int-95). MS (ESI) [M+H]⁺: m/z 287.

### Intermediate 96-1:4-bromo-6-methyl-1-(tetrahydro-2H-pyran-2-yl)-6,7-dihydrocyclopenta[f]indazol-5 (1H)-one (Int-96-6) & Intermediate 96-2:4-bromo-6,6-dimethyl-1-(tetrahydro-2H-pyran-2-yl)-6,7-dihydrocyclopenta[f]indazol-5 (1H)-one (Int-96-2)

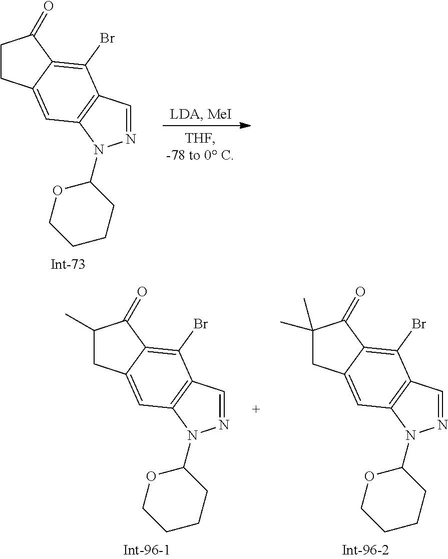

LDA (8.10 mL, 8.10 mmol, 1 M in THF/hexanes) was added to a solution of 4-bromo-1-(tetrahydro-2H-pyran-2-yl)-6,7-dihydrocyclopenta[f]indazol-5 (1H)-one (Int-73) (1.80 g, 5.37 mmol) in THF (130 mL) at −78° C. The reaction mixture was stirred at −78° C. for 1 h. After 1 h, the mixture was warmed up to 0° C. and stirred for 0.5 h. The reaction mixture was cooled to −78° C. and iodomethane (0.4 mL, 6.41 mmol) was added to the solution. After stirring at 0° C. for 2 h, the mixture was diluted with EtOAc, washed with sat. aq. NH₄Cl and brine, and the organic layer was dried over Na₂SO₄, filtered, and concentrated under reduced pressure. The mixture was filtered and concentrated in vacuo. The residue was purified by silica gel chromatography (0 to 100% EtOAc in hexane) affording 4-bromo-6-methyl-1-(tetrahydro-2H-pyran-2-yl)-6,7-dihydrocyclopenta[f]indazol-5 (1H)-one (Int-96-1) [MS (ESI): m/z (M+H)⁺ 349, 351] and 4-bromo-6,6-dimethyl-1-(tetrahydro-2H-pyran-2-yl)-6,7-dihydrocyclopenta[f]indazol-5 (1H)-one (Int-96-2) [MS (ESI): m/z (M+H)⁺ 363, 365].

### Intermediate 97: (6-methyl-1-(tetrahydro-2H-pyran-2-yl)-1,5,6,7-tetrahydrocyclopenta[f]indazol-4-yl)boronic acid (Int-97)

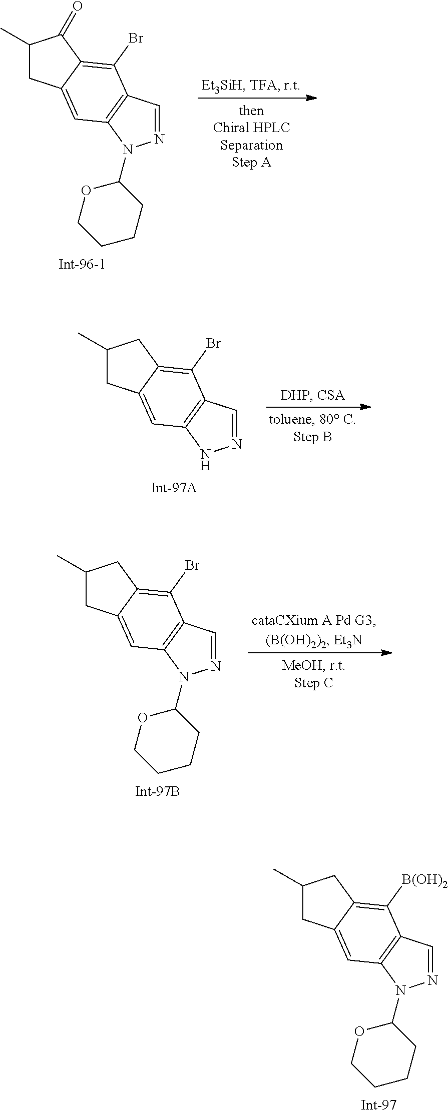

### Step A: 4-bromo-6-methyl-1,5,6,7-tetrahydrocyclopenta[f]indazole (Int-97A)

Trietbylsilane (8.10 mL, 8.10 mmol) was added to 4-bromo-6-methyl-1-(tetrahydro-2H-pyran-2-yl)-6,7-dihydrocyclopenta[f]indazol-5 (1H)-one (Int-96-1) (130 mg, 0.37 mmol) in TFA (3.6 mL) at room temperature. After stirring at room temperature for 48 h, the mixture was diluted with CHCl₃, washed with sat. aq. NaHCO₃ and brine, dried over Na₂SO₄, filtered, and concentrated. The residue was purified by silica gel chromatography (0 to 100% EtOAc in hexane) affording racemic 4-bromo-6-methyl-1,5,6,7-tetrahydrocyclopenta[f]indazole. Racemic 4-bromo-6-methyl-1,5,6,7-tetrahydrocyclopenta[f]indazole was separated by preparative chiral HPLC (Column S, hexane/EtOH=95/5) to afford 4-bromo-6-methyl-1,5,6,7-tetrahydrocyclopenta[f]indazole (Int-97A, 1st-eluting isomer). MS (ESI): m/z (M+H)⁺ 251, 253.

### Step B: 4-bromo-6-methyl-1-(tetrahydro-2H-pyran-2-yl)-1,5,6,7-tetrahydrocyclopenta[f]indazole (Int-97B)

3,4-dihydro-2H-pyran (9.6 μL, 0.105 mmol) and (1R)-(−)-camphor-10-sulfonic acid (3.66 mg, 0.0158 mmol) were added to a solution of 4-bromo-6-methyl-1,5,6,7-tetrahydrocyclopenta[f]indazole (93 mg, 0.37 mmol) (Int-97A) in toluene (0.8 mL), The mixture was stirred at 80° C. for 1 h. The solution was cooled to room temperature and diluted with EtOAc. The layers were separated and the organic phase was washed with sat. aq. NaHCO₃ and brine. The organic layer was dried over Na₂SO₄, filtered, and and concentrated. The residue was purified by silica gel chromatography (0 to 100% EtOAc in hexanes) to afford 4-bromo-6-methyl-1-(tetrahydro-2H-pyran-2-yl)-1,5,6,7-tetrahydrocyclopenta[f]indazole (Int-97B). MS (ESI): m/z (M+H)⁺ 335, 337.

### Step C: (6-methyl-1-(tetrahydro-2H-pyran-2-yl)-1,5,6,7-tetrahydrocyclopenta[f]indazol-4-yl)boronic acid (Int-97)

Tetrahydroxydiboron (12.3 mg, 0.137 mmol), cataCXium A Pd G3 (3.3 mg, 0.0046 mmol), and Et₃N (25 μL, 0.183 mmol) were added to a solution of 4-bromo-6-methyl-1-(tetrahydro-2H-pyran-2-yl)-1,5,6,7-tetrahydrocyclopenta[f]indazole (Int-97B) (15.3 mg, 0.046 mmol) in MeOH (0.91 mL). After stirring at room temperature for 1 h, the solution was filtered through a pad of CELITE®, and the filtrate was concentrated under reduced pressure. The residue was purified silica gel chromatography (0 to 20% EtOH in CHCl₃) affording (6-methyl-1-(tetrahydro-2H-pyran-2-yl)-1,5,6,7-tetrahydrocyclopenta[f]indazol-4-yl)boronic acid (Int-97). MS (ESI): m/z (M+H)⁺ 301.

The compound in the table below was synthesized using a similar procedure as described in the synthesis of Int-97 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-98 | 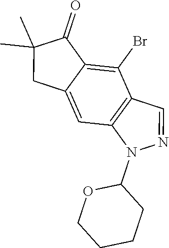 [SMILES: CC1(C)Cc2cc3c(cnn3C3CCCCO3)c(Br)c2C1=O] | 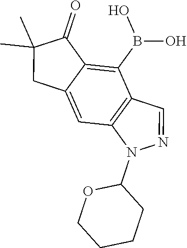 [SMILES: CC1(C)Cc2cc3c(cnn3C3CCCCO3)c(B(O)O)c2C1=O] | (6,6-dimethyl-1- (tetrahydro-2H- pyran-2-yl)-1,5,6,7- tetrahydrocyclopenta [f]indazol-4-yl) boronic acid | 315 |
| | Int-96-2 | | | |

### Intermediate 99: (5-cyano-1-(tetrahydro-2H-pyran-2-yl)-1,5,6,7-tetrahydrocyclopenta[f]indazol-4-yl)boronic acid (Int-99)

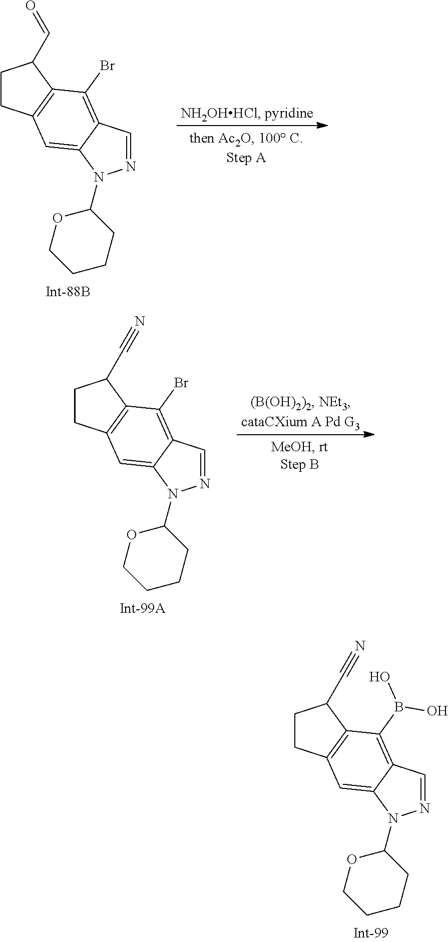 [SMILES: O=CC1CCc2cc3c(cnn3C3CCCCO3)c(Br)c21 ; "NH2OH·HCl, pyridine" ; N#CC1CCc2cc3c(cnn3C3CCCCO3)c(Br)c21 ; "(B(OH)2)2, NEt3,cataCXium A Pd G3" ; N#CC1CCc2cc3c(cnn3C3CCCCO3)c(B(O)O)c21]

### Step A: 4-bromo-1-(tetrahydro-2H-pyran-2-yl)-1,5,6,7-tetrahydrocyclopenta[f]indazole-5-carbonitrile (Int-99A)

A mixture of 4-bromo-1-(tetrahydro-2H-pyran-2-yl)-1,5,6,7-tetrahydrocyclopenta[f]indazole-5-carbaldehyde (Int-88B) (90.0 mg, 0.258 mmol) and hydroxylamine hydrochloride (23.3 mg, 0.335 mmol) in pyridine (1.00 mL) was stirred at room temperature for 30 min. After 30 min, acetic anhydride (1.00 mL, 10.7 mmol) was added and the reaction was heated to 100° C. for 30 min. The mixture was diluted with EtOAc and sat. aq. NaHCO₃. The layers were shaken and separated and the aqueous phase was extracted with EtOAc. The combined organic layers were washed with brine, dried over Na₂SO₄, filtered, and concentrated. The residue was purified via silica gel chromatography (0 to 80% EtOAc in hexane) to give 4-bromo-1-(tetrahydro-2H-pyran-2-yl)-1,5,6,7-tetrahydrocyclopenta[f]indazole-5-carbonitrile (Int-99A). MS (ESI): m/z (M+H)⁺ 346, 348.

### Step B: (5-cyano-1-(tetrahydro-2H-pyran-2-yl)-1,5,6,7-tetrahydrocyclopenta[f]indazol-4-yl)boronic acid (Int-99)

A mixture of 4-bromo-1-(tetrahydro-2H-pyran-2-yl)-1,5,6,7-tetrahydrocyclopenta[f]indazole-5-carbonitrile (Int-99A) (78.0 mg, 0.225 mmol), tetrahydroxydiboron (60.6 mg, 0.676 mmol), triethylamine (126 μL, 0.901 mmol) and cataCXium A Pd G3 (16.4 mg, 0.0225 mmol) in MeOH (3.90 mL) was stirred at room temperature for 3 h. The reaction was filtered through a pad of CELITE® and washed with MeOH. The filtrate was concentrated and the residue was purified via silica gel chromatography (10 to 100% EtOAc in hexane) to give (5-cyano-1-(tetrahydro-2H-pyran-2-yl)-1,5,6,7-tetrahydrocyclopenta[f]indazol-4-yl)boronic acid (Int-99) (61.1 mg, 0.196 mmol). MS (ESI): m/z (M+H)⁺ 312.

### Intermediate 100: (6,6-difluoro-5-methyl-1-(tetrahydro-2H-pyran-2-yl)-1,5,6,7-tetrahydrocyclopenta[f]indazol-4-yl)boronic acid (Int-100)

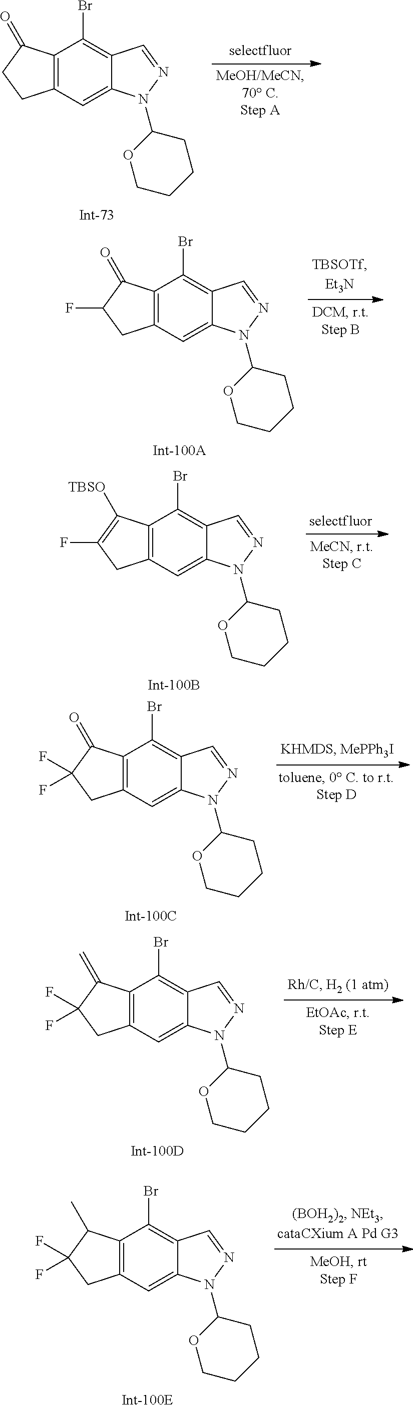 [SMILES: O=C1CCc2cc3c(cnn3C3CCCCO3)c(Br)c21 ; O=C1c2c(cc3c(cnn3C3CCCCO3)c2Br)CC1F ; CC(C)(C)[Si](C)(C)OC1=C(F)Cc2cc3c(cnn3C3CCCCO3)c(Br)c21 ; O=C1c2c(cc3c(cnn3C3CCCCO3)c2Br)CC1(F)F ; C=C1c2c(cc3c(cnn3C3CCCCO3)c2Br)CC1(F)F ; CC1c2c(cc3c(cnn3C3CCCCO3)c2Br)CC1(F)F]

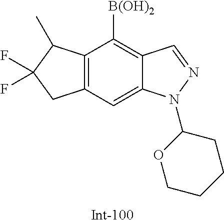 [SMILES: CC1c2c(cc3c(cnn3C3CCCCO3)c2B(O)O)CC1(F)F]

### Step A: 4-bromo-6-fluoro-1-(tetrahydro-2H-pyran-2-yl)-6,7-dihydrocyclopenta[f]indazol-5 (1H)-one (Int-100A)

4-bromo-1-(tetrahydro-2H-pyran-2-yl)-6,7-dihydrocyclopenta[f]indazol-5 (1H)-one (Int-73) (5.00 g, 14.9 mmol) and selectfluor (15.8 g, 44.7 mmol) were dissolved in MeOH (150 mL) and MeCN (150 mL) and stirred for 1.5 h at 70° C. The mixture was cooled to room temperature, quenched with sat. aq. NaHCO₃, and diluted with CHCl₃. The organic layer was separated, washed with brine, dried over Na₂SO₄, filtered, and the filtrate was concentrated under reduced pressure. The residue obtained was purified by silica gel chromatography (hexane/EtOAc=50/50 to 0/100, then MeOH/CHCl_{3=0/100} to 50/50) to afford 4-bromo-6-fluoro-1-(tetrahydro-2H-pyran-2-yl)-6,7-dihydrocyclopenta[f]indazol-5(1H)-one (Int-100A). MS (ESI) [M+H]⁺: m/z 353, 355.

### Step B: 4-bromo-5-((tert-butyldimethylsilyl)oxy)-6-fluoro-1-(tetrahydro-2H-pyran-2-yl)-1,7-dihydrocyclopenta[f]indazole (Int-100B)

TBSOTf (5.70 mL, 25.0 mmol) was added to a mixture of 4-bromo-6-fluoro-1-(tetrahydro-2H-pyran-2-yl)-6,7-dihydrocyclopenta[f]indazol-5 (1H)-one (Int-100A) (2.50 g, 7.10 mmol) and triethylamine (9.90 mL, 71.0 mmol) in DCM (35 mL) at room temperature. The reaction was stirred for 30 min at room temperature. The reaction was quenched with sat. aq. NaHCO₃ and diluted with CHCl₃. The organic layer was separated, washed with brine, and the combined organics were dried over Na₂SO₄, filtered, and the filtrate was concentrated under reduced pressure to afford 4-bromo-5-((tert-butyldimethylsilyl)oxy)-6-fluoro-1-(tetrahydro-2H-pyran-2-yl)-1,7-dihydrocyclopenta[f]indazole (Int-100B), which was used directly in the next step without further purification. MS (ESI) [M+H]⁺: m/z 467, 469.

### Step C: 4-bromo-6,6-difluoro-1-(tetrahydro-2H-pyran-2-yl)-6,7-dihydrocyclopenta[f]indazol-5 (1H)-one (Int-100C)

Selectfluor (1.72 g, 18.7 mmol) was added to a mixture of 4-bromo-5-((tert-butyldimethylsilyl)oxy)-6-fluoro-1-(tetrahydro-2H-pyran-2-yl)-1,7-dihydrocyclopenta[f]indazole (3.32 g, 7.10 mmol) (Int-100B) in MeCN (18.7 mL) at room temperature and stirred for 10 min. The mixture was quenched with sat. aq. NaHCO₃ and diluted with CHCl₃. The organic layer was separated, washed with brine, and dried over Na₂SO₄. The dried solution was filtered and the filtrate was concentrated under reduced pressure. The residue obtained was purified by silica gel chromatography (hexane/EtOAc=100/0 to 20/80) to afford 4-bromo-6,6-difluoro-1-(tetrahydro-2H-pyran-2-yl)-6,7-dihydrocyclopenta[f]indazol-5 (1H)-one (Int-100C). ¹H-NMR (400 MHz, CDCl₃) δ 8.26 (s, 1H), 7.59 (s, 1H), 5.78-5.74 (dd, J=8.8, 2.2 Hz, 1H), 4.04-4.00 (m, 1H), 3.81-3.75 (m, 1H), 3.67 (t, J=13.3, 2H), 2.55-2.47 (m, 1H), 2.23-2.14 (m, 2H), 1.87-1.72 (m, 3H). MS (ESI) [M+H]⁺: m/z 371, 373.

### Step D: 4-bromo-6,6-difluoro-5-methylene-1-(tetrahydro-2H-pyran-2-yl)-1,5,6,7-tetrahydrocyclopenta[f]indazole (Int-100D)

KHMDS (2.58 mL, 2.58 mmol, 1 M in THF) was added to a solution of methyltriphenylphosphonium iodide (1.05 g, 2.59 mmol) in toluene (4 mL) at 0° C. After stirring for 30 min, a solution of 4-bromo-6,6-difluoro-1-(tetrahydro-2H-pyran-2-yl)-6,7-dihydrocyclopenta[f]indazol-5 (1H)-one (Int-100C) (600 mg, 1.62 mmol) in toluene (4 mL) was added dropwise at room temperature and stirred for 1 h. The mixture was quenched with sat. aq. NH₄Cl and diluted with CHCl₃. The organic layer was separated, washed with brine, dried over Na₂SO₄, filtered, and the filtrate was concentrated under reduced pressure. The residue was purified by silica gel chromatography (hexane/EtOAc=90/10 to 20/80) to afford 4-bromo-6,6-difluoro-5-methylene-1-(tetrahydro-2H-pyran-2-yl)-1,5,6,7-tetrahydrocyclopenta[f]indazole (Int-100D). ¹H-NMR (400 MHz, CDCl₃) δ 8.05 (s, 1H), 7.45 (s, 1H), 6.68 (t, J=2.4, 1H), 5.89-5.84 (m, 1H), 5.69 (dd, J=9.2, 2.3 Hz, 1H), 4.04-4.00 (m, 1H), 3.79-3.71 (m, 1H), 3.65-3.44 (m, 2H), 2.57-2.48 (m, 1H), 2.20-2.07 (m, 2H), 1.90-1.63 (m, 3H). MS (ESI) [M+H]⁺: 369, 371.

### Step E: 4-bromo-6,6-difluoro-5-methyl-1-(tetrahydro-2H-pyran-2-yl)-1,5,6,7-tetrahydrocyclopenta[f]indazole (Int-100E)

4-bromo-6,6-difluoro-5-methylene-1-(tetrahydro-2H-pyran-2-yl)-1,5,6,7-tetrahydrocyclopenta[f]indazole (Int-100D) (100 mg, 0.27 mmol) was dissolved in EtOAc (2.7 mL). The reaction was purged with nitrogen and Rh/C (250 mg, 5 wt %) was added. The atmosphere was replaced with hydrogen (1 atm) via balloon and the reaction mixture was stirred at room temperature for 1 h. The mixture was filtered through a pad of CELITE® and the filtrate was concentrated in vacuo to afford 4-bromo-6,6-difluoro-5-methyl-1-(tetrahydro-2H-pyran-2-yl)-1,5,6,7-tetrahydrocyclopenta[f]indazole (Int-100E), which was used directly in the next step without further purification. MS (ESI) [M+H]⁺: m/z 371, 373.

### Step F: (6,6-difluoro-5-methyl-1-(tetrahydro-2H-pyran-2-yl)-1,5,6,7-tetrahydrocyclopenta[f]indazol-4-yl)boronic acid (Int-100)

Tetrahydroxydiboron (130 mg, 1.45 mmol), triethylamine (0.41 mL, 2.90 mmol), and cataXCium A Pd G3 (53.0 mg, 0.072 mmol) were added to a solution of 4-bromo-6,6-difluoro-5-methyl-1-(tetrahydro-2H-pyran-2-yl)-1,5,6,7-tetrahydrocyclopenta[f]indazole (Int-100E) (90 mg, 0.24 mmol) in MeOH (1.2 mL). The mixture was stirred at room temperature for 30 min under N₂. The mixture was filtered through a pad of CELITE®, and the filtrate was concentrated under reduced pressure. The residue was purified by flash silica gel chromatography (hexane/EtOAc=95/5 to 0/100) to afford (6,6-difluoro-5-methyl-1-(tetrahydro-2H-pyran-2-yl)-1,5,6,7-tetrahydrocyclopenta[f]indazol-4-yl)boronic acid (Int-100). MS (ESI) [M+H]⁺: m/z 337.

### Intermediate 101: (6,6-difluoro-5,7,7-trimethyl-1-(tetrahydro-2H-pyran-2-yl)-1,5,6,7-tetrahydrocyclopenta[f]indazol-4-yl)boronic acid (Int-101)

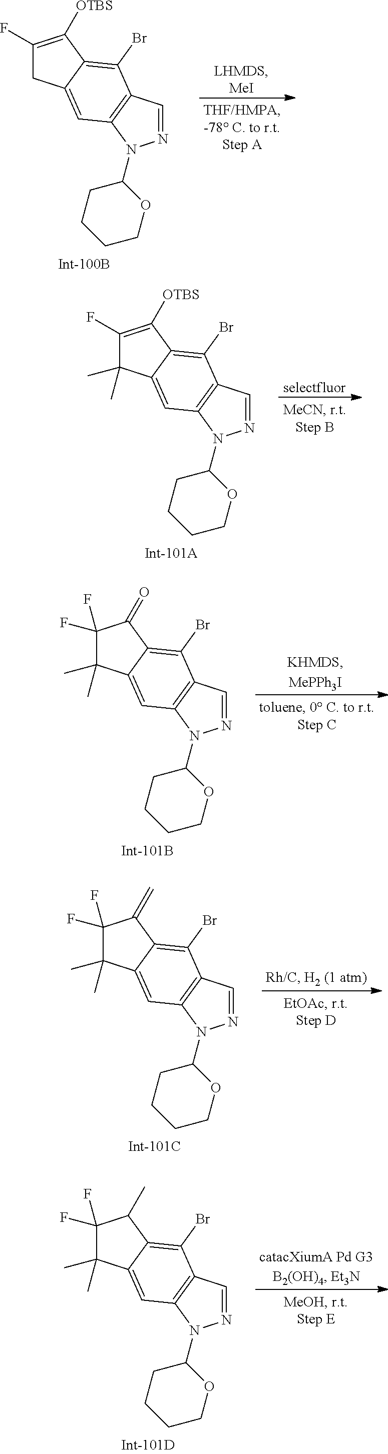

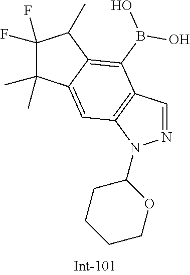 [SMILES: CC1c2c(cc3c(cnn3C3CCCCO3)c2B(O)O)C(C)(C)C1(F)F]

### Step A: 4-bromo-5-((tert-butyldimethylsilyl)oxy)-6-fluoro-7,7-dimethyl-1-(tetrahydro-2H-pyran-2-yl)-1,7-dihydrocyclopenta[f]indazole (Int-101A)

LiHMDS (0.99 mL, 0.99 mmol, 1 M in THF) was added to a mixture of 4-bromo-5-((tert-butyldimethylsilyl)oxy)-6-fluoro-1-(tetrahydro-2H-pyran-2-yl)-1,7-dihydrocyclopenta[f]indazole (Int-100B) (210 mg, 0.449 mmol) and iodomethane (0.22 mL, 3.59 mmol) in THF (2.2 mL) and HMPA (0.2 mL) at −78° C. The mixture was warmed to room temperature and stirred for 30 min under N₂. The mixture was diluted with sat. aq. NaHCO₃ and EtOAc. The layers were separated and the organic layer was dried over Na₂SO₄, filtered, and concentrated to afford 4-bromo-5-((tert-butyldimethylsilyl)oxy)-6-fluoro-7,7-dimethyl-1-(tetrahydro-2H-pyran-2-yl)-1,7-dihydrocyclopenta[f]indazole (Int-101A), which was used directly in the next step without further purification. MS (ESI) [M+H]⁺: m/z 495, 497.

### Step B: 4-bromo-6,6-difluoro-7,7-dimethyl-1-(tetrahydro-2H-pyran-2-yl)-6,7-dihydrocyclopenta[f]indazol-5 (1H)-one (Int-101B)

Selectfluor (191 mg, 0.539 mmol) was added to a solution of 4-bromo-5-((tert-butyldimethylsilyl)oxy)-6-fluoro-7,7-dimethyl-1-(tetrahydro-2H-pyran-2-yl)-1,7-dihydrocyclopenta[f]indazole (Int-101A) in MeCN (2.2 mL). The mixture was stirred at room temperature for 20 min. CHCl₃ and sat. aq. NaHCO₃ was added and the layers were separated. The organic layer was washed with brine, dried over Na₂SO₄, filtered, and concentrated. The residue obtained was purified by column chromatography (hexanes/EtOAc=100/0 to 20/80) to afford 4-bromo-6,6-difluoro-7,7-dimethyl-1-(tetrahydro-2H-pyran-2-yl)-6,7-dihydrocyclopenta[f]indazol-5 (1H)-one (Int-101B). MS (ESI) [M+H]⁺: m/z 399.

### Step C: 4-bromo-6,6-difluoro-7,7-dimethyl-5-methylene-1-(tetrahydro-2H-pyran-2-yl)-1,5,6,7-tetrahydrocyclopenta[f]indazole (Int-101C)

KHMDS (0.34 mL, 0.335 mmol, 1 M in THF) was added to a solution of methyltriphenylphosphonium iodide (136 mg, 0.336 mmol) in toluene (0.8 mL) at 0° C. After the reaction mixture was stirred at that temperature for 30 min, a solution of 4-bromo-6,6-difluoro-7,7-dimethyl-1-(tetrahydro-2H-pyran-2-yl)-6,7-dihydrocyclopenta[f]indazol-5 (1H)-one (Int-101B) (67 mg, 0.168 mmol) in toluene (0.8 mL) was added dropwise and the reaction was stirred for 1 h at room temperature. The mixture was quenched with sat. aq. NH₄Cl and diluted with EtOAc. The organic layer was separated, washed with brine and dried over Na₂SO₄. The dried solution was filtered and the filtrate was concentrated under reduced pressure. The residue obtained was purified by column chromatography (hexane/EtOAc=100/0 to 40/60) to afford 4-bromo-6,6-difluoro-7,7-dimethyl-5-methylene-1-(tetrahydro-2H-pyran-2-yl)-1,5,6,7-tetrahydrocyclopenta[f]indazole (Int-101C). MS (ESI) [M+H]⁺: m/z 397, 399.

### Step D: 4-bromo-6,6-difluoro-5,7,7-trimethyl-1-(tetrahydro-2H-pyran-2-yl)-1,5,6,7-tetrahydrocyclopenta[f]indazole (Int-101D)

4-bromo-6,6-difluoro-7,7-dimethyl-5-methylene-1-(tetrahydro-2H-pyran-2-yl)-1,5,6,7-tetrahydrocyclopenta[f]indazole (Int-101C) (28 mg, 0.070 mmol) was dissolved in EtOAc (0.7 mL). The reaction was purged with N₂ and Rh on carbon (224 mg) was added. The atmosphere was replaced with hydrogen and the reaction mixture was stirred at room temperature for 3 h. The reaction was filtered and concentrated in vacuo to afford 4-bromo-6,6-difluoro-5,7,7-trimethyl-1-(tetrahydro-2H-pyran-2-yl)-1,5,6,7-tetrahydrocyclopenta[f]indazole (Int-101D), which was used directly in the next step without further purification. MS (ESI) [M+H]⁺: m/z 399, 401.

### Step E: (6,6-difluoro-5,7,7-trimethyl-1-(tetrahydro-2H-pyran-2-yl)-1,5,6,7-tetrahydrocyclopenta[f]indazol-4-yl)boronic acid (Int-101)

Tetrahydroxydiboron (25.6 mg, 0.286 mmol)), triethylamine (0.080 mL, 0.571 mmol), and cataXCium A Pd G3 (10.4 mg, 0.014 mmol) were added to a solution of 4-bromo-6,6-difluoro-5,7,7-trimethyl-1-(tetrahydro-2H-pyran-2-yl)-1,5,6,7-tetrahydrocyclopenta[f]indazole (Int-101D) (19 mg, 0.048 mmol) in MeOH (0.5 mL). The mixture was stirred at room temperature for 30 min under N₂. The reaction mixture was filtered and concentrated under reduced pressure. The resulting crude residue was purified by flash silica gel chromatography (hexane/EtOAc=100/0 to 80/20) to afford (6,6-difluoro-5,7,7-trimethyl-1-(tetrahydro-2H-pyran-2-yl)-1,5,6,7-tetrahydrocyclopenta[f]indazol-4-yl)boronic acid (Int-101). MS (ESI) [M+H]⁺: m/z 365.

### Intermediate 102: (5-ethyl-5,6,6-trifluoro-1-(tetrahydro-2H-pyran-2-yl)-1,5,6,7-tetrahydrocyclopenta[f]indazol-4-yl)boronic acid (Int-102)

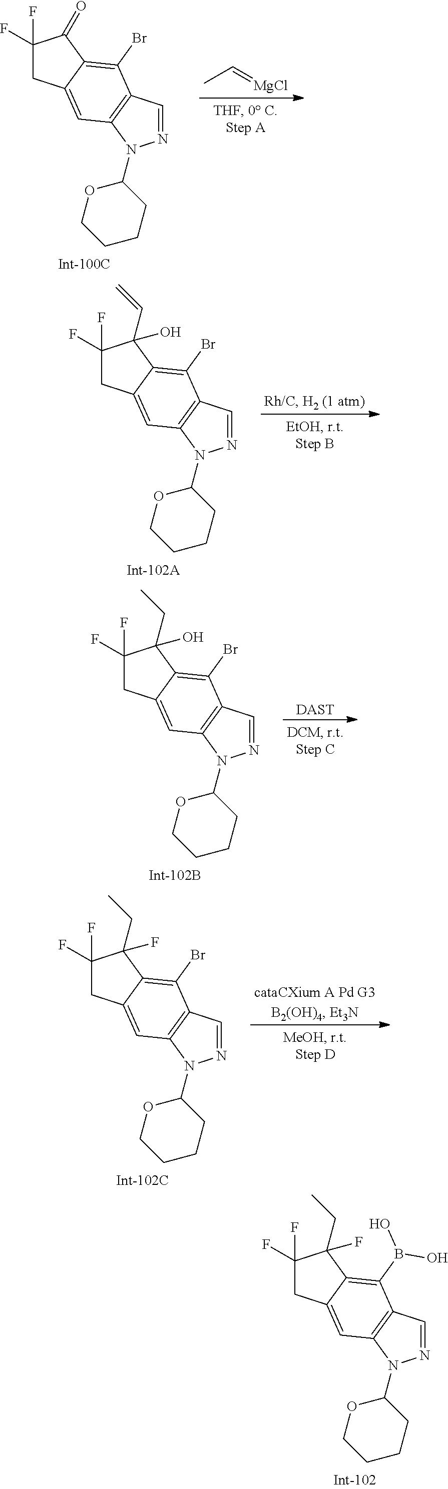

### Step A: 4-bromo-6,6-difluoro-1-(tetrahydro-2H-pyran-2-yl)-5-vinyl-1,5,6,7-tetrahydrocyclopenta[f]indazol-5-ol (Int-102A)

Vinylmagnesium bromide (0.296 mL, 0.296 mmol, 1 M in THF) was added to a solution of 4-bromo-6,6-difluoro-1-(tetrahydro-2H-pyran-2-yl)-6,7-dihydrocyclopenta[f]indazol-5 (1H)-one (Int-100C) (100 mg, 0.269 mmol) in THF (2.70 mL) at 0° C. The mixture was stirred at 0° C. for 10 min. To a mixture was added additional vinylmagnesium bromide (0.100 mL, 0.100 mmol, 1.0 M in THF) and the mixture was stirred at 0° C. for 10 min. The mixture was diluted with EtOAc and 6N HCl (0.5 mL), then extracted with EtOAc three times. The combined organic layers were washed with brine, dried over Na₂SO₄, filtered, and concentrated. The residue was purified via silica gel chromatography (20 to 60% EtOAc in hexane) to give 4-bromo-6,6-difluoro-1-(tetrahydro-2H-pyran-2-yl)-5-vinyl-1,5,6,7-tetrahydrocyclopenta[f]indazol-5-ol (Int-102A). MS (ESI): m/z (M+H)⁺ 399, 401. ¹H NMR (400 MHz, CDCl₃) δ 8.05 (s, 1H), 7.48-7.43 (m, 1H), 6.13-6.05 (m, 1H), 5.71-5.67 (m, 1H), 5.43-5.40 (m, 1H), 5.22-5.17 (m, 1H), 4.03-3.98 (m, 1H), 3.77-3.72 (m, 1H), 3.58-3.35 (m, 2H), 2.93-2.87 (m, 1H), 2.57-2.45 (m, 1H), 2.20-2.07 (m, 2H), 1.79-1.68 (m, 3H).

### Step B: 4-bromo-5-ethyl-6,6-difluoro-1-(tetrahydro-2H-pyran-2-yl)-1,5,6,7-tetrahydrocyclopenta[f]indazol-5-ol (Int-102B)

A mixture of 4-bromo-6,6-difluoro-1-(tetrahydro-2H-pyran-2-yl)-5-vinyl-1,5,6,7-tetrahydrocyclopenta[f]indazol-5-ol (Int-102A) (1010 mg, 0.274 mmol) and rhodium on carbon (273.8 mg, 5 wt %) in EtOH (2.74 mL) was evacuated and backfilled with an H₂ balloon (3×). After stirring at room temperature for 1 h, the mixture was filtered through a CELITE® pad washed with MeOH and the filtrate was concentrated to give 4-bromo-5-ethyl-6,6-difluoro-1-(tetrahydro-2H-pyran-2-yl)-1,5,6,7-tetrahydrocyclopenta[f]indazol-5-ol (Int-102B). MS (ESI): m/z (M+H)⁺ 401, 403. ¹H NMR (400 MHz, CDCl₃) δ 8.06-8.05 (m, 1H), 7.40-7.38 (m, 1H), 5.70-5.65 (m, 1H), 4.03-3.97 (m, 1H), 3.77-3.71 (m, 1H), 3.56-3.34 (m, 2H), 2.70-2.68 (m, 1H), 2.54-2.47 (m, 1H), 2.29-2.06 (m, 4H), 1.79-1.66 (m, 3H), 0.94-0.89 (m, 3H).

### Step C: 4-bromo-5-ethyl-5,6,6-trifluoro-1-(tetrahydro-2H-pyran-2-yl)-1,5,6,7-tetrahydrocyclopenta[f]indazole (Int-102C)

Diethylaminosulfur trifluoride (0.075 mL, 0.57 mmol) was added to a solution of 4-bromo-5-ethyl-6,6-difluoro-1-(tetrahydro-2H-pyran-2-yl)-1,5,6,7-tetrahydrocyclopenta[f]indazol-5-ol (Int-102B) (103 mg, 0.257 mmol) in DCM (2.57 mL) at room temperature. The mixture was stirred at room temperature for 20 min and diluted with sat. aq. NaHCO₃. The mixture was extracted with CHCl₃ (3×) and the combined organic layers were washed with brine, dried over Na₂SO₄, filtered, and concentrated. The residue was purified via silica gel chromatography (0 to 50% EtOAc in hexane) to give 4-bromo-5-ethyl-5,6,6-trifluoro-1-(tetrahydro-2H-pyran-2-yl)-1,5,6,7-tetrahydrocyclopenta[f]indazole (Int-102C). MS (ESI): m/z (M+H)⁺ 403, 405. ¹H NMR (400 MHz, CDCl₃) δ 8.08-8.07 (m, 1H), 7.44-7.41 (m, 1H), 5.71-5.66 (m, 1H), 4.02-3.97 (m, 1H), 3.77-3.71 (m, 1H), 3.59-3.53 (m, 1H), 3.43-3.36 (m, 1H), 2.54-2.38 (m, 3H), 2.18-2.07 (m, 2H), 1.80-1.67 (m, 3H), 1.01-0.97 (m, 3H).

### Step D: (5-ethyl-5,6,6-trifluoro-1-(tetrahydro-2H-pyran-2-yl)-1,5,6,7-tetrahydrocyclopenta[f]indazol-4-yl)boronic acid (Int-102)

A mixture of 4-bromo-5-ethyl-5,6,6-trifluoro-1-(tetrahydro-2H-pyran-2-yl)-1,5,6,7-tetrahydrocyclopenta[f]indazole (Int-102C) (77.1 mg, 0.191 mmol), tetrahydroxydiboron (137 mg, 1.53 mmol), triethylamine (266 μL, 1.91 mmol) and cataCXium A Pd G3 (42 mg, 0.057 mmol) in MeOH (1.91 mL) was stirred at room temperature for 50 min. The mixture was diluted with EtOAc and water, then extracted with EtOAc (3×). The combined organic layers were washed with brine, dried over Na₂SO₄, filtered, and concentrated. The residue was purified via silica gel chromatography (30 to 100% EtOAc in hexane) to give (5-ethyl-5,6,6-trifluoro-1-(tetrahydro-2H-pyran-2-yl)-1,5,6,7-tetrahydrocyclopenta[f]indazol-4-yl)boronic acid (Int-102). MS (ESI): m/z (M+H)⁺ 369. ¹H NMR (400 MHz, CDCl₃) δ 8.41-8.39 (m, 1H), 7.53-7.42 (m, 1H), 5.72-5.67 (m, 1H), 4.03-3.92 (m, 1H), 3.78-3.70 (m, 1H), 3.66-3.51 (m, 1H), 3.49-3.35 (m, 1H), 2.59-2.48 (m, 1H), 2.26-2.01 (m, 4H), 1.80-1.64 (m, 3H), 1.42-1.37 (m, 2H), 1.14-0.96 (m, 3H).

The compound in the table below was synthesized using a similar procedure as described in the synthesis of Int-102 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-103 | 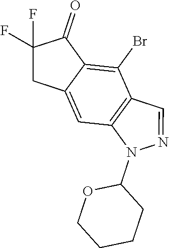 [SMILES: O=C1c2c(cc3c(cnn3C3CCCCO3)c2Br)CC1(F)F] | 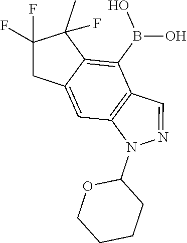 [SMILES: CC1(F)c2c(cc3c(cnn3C3CCCCO3)c2B(O)O)CC1(F)F] | (5,6,6-trifluoro-5- methyl-1-(tetrahydro- 2H-pyran-2-yl)- 1,5,6,7- tetrahydrocyclopenta [f]indazol-4-yl) boronic acid | 355 |
| | Int-100C | | | |

### Intermediate 104: (5-ethyl-6,6-difluoro-1-(tetrahydro-2H-pyran-2-yl)-1,5,6,7-tetrahydrocyclopenta[f]indazol-4-yl)boronic acid (Int-104)

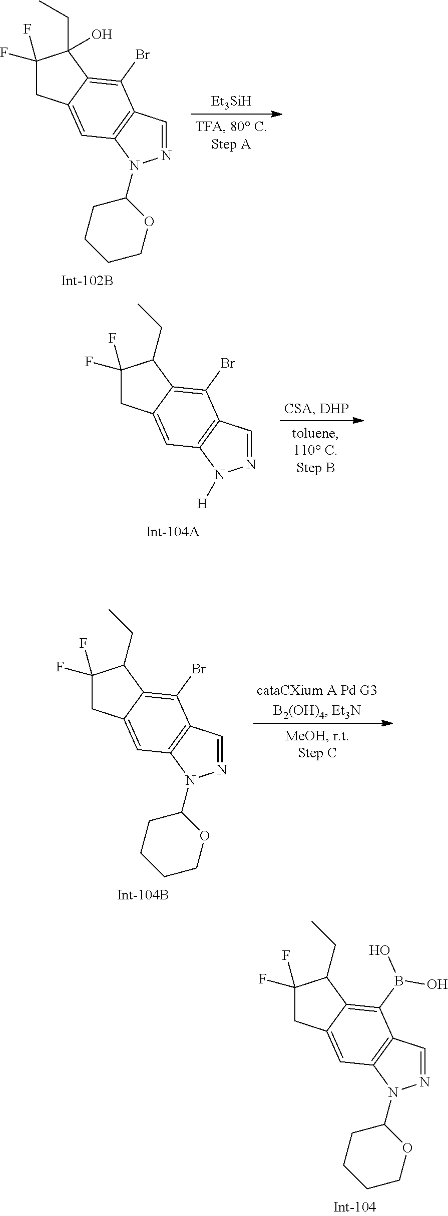

### Step A: 4-bromo-5-ethyl-6,6-difluoro-1,5,6,7-tetrahydrocyclopenta[f]indazole (Int-104A)

Trietbylsilane (1.79 mL, 11.2 mmol) was added to a solution of 4-bromo-5-ethyl-6,6-difluoro-1-(tetrahydro-2H-pyran-2-yl)-1,5,6,7-tetrahydrocyclopenta[f]indazol-5-ol (Int-102B) (300 mg, 0.750 mmol) in TFA (2.8 mL) at room temperature. The reaction mixture was warmed to 80° C. and stirred for 5 h. The reaction mixture was cooled to room temperature and quenched with sat. aq. NaHCO₃. The reaction mixture was extracted with CHCl₃, the organic layer was separated, dried over Na₂SO₄, filtered, and concentrated in vacuo. The residue was purified by silica gel chromatography (0 to 100% EtOAc in hexanes) to give 4-bromo-5-ethyl-6,6-difluoro-1,5,6,7-tetrahydrocyclopenta[f]indazole (Int-104A). MS (ESI) [M+H]⁺: m/z 301, 303.

### Step B: 4-bromo-5-ethyl-6,6-difluoro-1-(tetrahydro-2H-pyran-2-yl)-1,5,6,7-tetrahydrocyclopenta[f]indazole (Int-104B)

3,4-dihydro-2H-pyran (54.0 μL, 0.60 mmol) and (+)-(1S)-10-camphorsulfonic acid (9.2 mg, 0.040 mmol) were added to a solution of 4-bromo-5-ethyl-6,6-difluoro-1,5,6,7-tetrahydrocyclopenta[f]indazole (Int-104A) (60.0 mg, 0.199 mmol) in toluene (2.0 mL). The mixture was warmed to 110° C. and stirred for 2 h. The solution was cooled to room temperature and diluted with EtOAc. The reaction mixture was washed with sat. aq. NaHCO₃ and brine, dried over Na₂SO₄, filtered, and evaporated. The residue was purified by silica gel chromatography (0 to 100% EtOAc in hexanes) to afford 4-bromo-5-ethyl-6,6-difluoro-1-(tetrahydro-2H-pyran-2-yl)-1,5,6,7-tetrahydrocyclopenta[f]indazole (Int-104B). MS (ESI) [M+H]⁺: m/z 385, 387.

### Step C: 5-ethyl-6,6-difluoro-1-(tetrahydro-2H-pyran-2-yl)-1,5,6,7-tetrahydrocyclopenta[f]indazol-4-yl)boronic acid (Int-104)

A mixture of 4-bromo-5-ethyl-6,6-difluoro-1-(tetrahydro-2H-pyran-2-yl)-1,5,6,7-tetrahydrocyclopenta[f]indazole (Int-104B) (80 mg, 0.21 mmol), tetrahydroxydiboron (56 mg, 0.62 mmol), cataXCium A Pd G3 (15 mg, 0.020 mmol), and Et₃N (0.12 mL, 0.83 mmol) in MeOH (4.1 mL) was stirred at room temperature for 2 h. The reaction mixture was filtered through a celite pad, washed with MeOH, and concentrated. The residue was purified by silica gel chromatography (20% MeOH in CHCl₃) to give 5-ethyl-6,6-difluoro-1-(tetrahydro-2H-pyran-2-yl)-1,5,6,7-tetrahydrocyclopenta[f]indazol-4-yl)boronic acid (Int-104). MS (ESI) [M+H]⁺: m/z 351.

### Intermediate 105: (6,6-difluoro-5-methoxy-1-(tetrahydro-2H-pyran-2-yl)-1,5,6,7-tetrahydrocyclopenta[f]indazol-4-yl)boronic acid (Int-105)

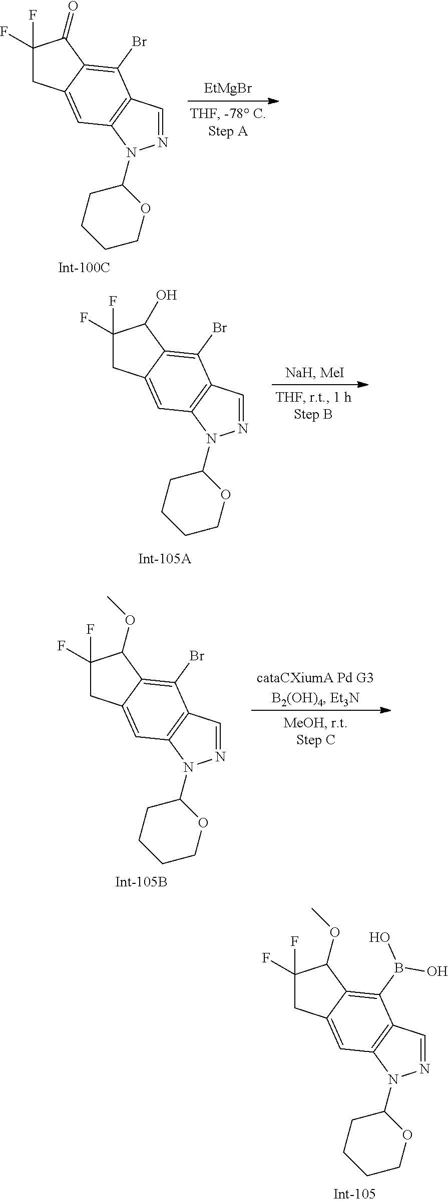

### Step A: 4-bromo-6,6-difluoro-1-(tetrahydro-2H-pyran-2-yl)-1,5,6,7-tetrahydrocyclopenta[f]indazol-5-ol (Int-105A)

Ethylmagnesium bromide (0.106 mL, 0.319 mmol, 3.0 M in Et₂₀) was added to a solution of 4-bromo-6,6-difluoro-1-(tetrahydro-2H-pyran-2-yl)-6,7-dihydrocyclopenta[f]indazol-5 (1H)-one (Int-100C) (91.2 mg, 0.246 mmol) in THF (2.46 mL) at −78° C. The mixture was stirred at −78° C. for 30 min. The mixture was quenched with HCl (0.800 mL, 2.0 M in H₂O) and extracted with EtOAc (3×). The combined organic layers were washed with brine, dried over Na₂SO₄, filtered, and concentrated. The residue was purified via silica gel chromatography (50 to 100% EtOAc in hexane) to give 4-bromo-6,6-difluoro-1-(tetrahydro-2H-pyran-2-yl)-1,5,6,7-tetrahydrocyclopenta[f]indazol-5-ol (Int-105A). MS (ESI): m/z (M+H)⁺ 373, 375. ¹H NMR (400 MHz, CDCl₃) δ 8.03 (s, 1H), 7.44-7.43 (m, 1H), 5.69 (td, J=9.0 Hz, 2.6 Hz, 1H), 5.18 (d, J=11.6 Hz, 1H), 4.01-3.96 (m, 1H), 3.77-3.65 (m, 2H), 3.54-3.44 (m, 1H), 2.54-2.46 (m, 2H), 2.18-2.04 (m, 2H), 1.80-1.67 (m, 3H).

### Step B: 4-bromo-6,6-difluoro-5-methoxy-1-(tetrahydro-2H-pyran-2-yl)-1,5,6,7-tetrahydrocyclopenta[f]indazole (Int-105B)

Methyl iodide (7.5 μL, 0.12 mmol) was added to a solution of 4-bromo-6,6-difluoro-1-(tetrahydro-2H-pyran-2-yl)-1,5,6,7-tetrahydrocyclopenta[f]indazol-5-ol (Int-105A) (30 mg, 0.080 mmol), and NaH (4.2 mg, 0.11 mmol, 60 wt %) in THF (0.800 mL) at room temperature. The mixture was stirred at room temperature for 1 h. Additional NaH (4.2 mg, 0.11 mmol, 60 wt %) and methyl iodide (7.5 μL, 0.12 mmol) were added after 1 h. After 6 b, the mixture was diluted with EtOAc and water, the layers were separated, and the aqueous phase was extracted with EtOAc (3×). The combined organic layers were washed with brine, dried over sodium sulfate, filtered, and concentrated. The residue was purified via silica gel chromatography (9 to 30% EtOAc in hexane) to give 4-bromo-6,6-difluoro-5-methoxy-1-(tetrahydro-2H-pyran-2-yl)-1,5,6,7-tetrahydrocyclopenta[f]indazole (Int-105B). MS (ESI): m/z (M+H)⁺ 387. ¹H NMR (400 MHz, CDCl₃) δ 8.03-8.02 (m, 1H), 7.42-7.40 (m, 1H), 5.70-5.66 (m, 1H), 4.72-4.69 (m, 1H), 3.99-3.95 (m, 1H), 3.78-3.64 (m, 5H), 3.49-3.35 (m, 1H), 2.56-2.45 (m, 1H), 2.19-2.05 (m, 2H), 1.79-1.66 (m, 3H).

### Step C: (6,6-difluoro-5-methoxy-1-(tetrahydro-2H-pyran-2-yl)-1,5,6,7-tetrahydrocyclopenta[f]indazol-4-yl)boronic acid (Int-105)

A mixture of 4-bromo-6,6-difluoro-5-methoxy-1-(tetrahydro-2H-pyran-2-yl)-1,5,6,7-tetrahydrocyclopenta[f]indazole (Int-105B) (11.1 mg, 0.0287 mmol), tetrahydroxydiboron (20.6 mg, 229 mmol), triethylamine (39.9 μL, 2.66 mmol), and cataCXium A Pd G3 (4.2 mg, 0.0057 mmol) in MeOH (0.290 mL) was stirred at room temperature for 50 min. The mixture was diluted with EtOAc and water, the layers were separated, and the aqueous phase was extracted with EtOAc three times. The combined organic layers were washed with brine, dried over sodium sulfate, filtered, and concentrated. The residue was purified via silica gel chromatography (0 to 10% MeOH in CHCl₃) to give (6,6-difluoro-5-methoxy-1-(tetrahydro-2H-pyran-2-yl)-1,5,6,7-tetrahydrocyclopenta[f]indazol-4-yl)boronic acid (Int-105). MS (ESI): m/z (M+H)⁺ 353. ¹H NMR (400 MHz, CDCl₃) δ 8.50-8.49 (m, 1H), 8.01-7.99 (m, 1H), 7.52-7.50 (m, 1H), 6.57-6.54 (m, 1H), 5.72-5.67 (m, 1H), 5.10-5.03 (m, 1H), 4.02-3.97 (m, 1H), 3.77-3.70 (m, 4H), 3.67-3.48 (m, 2H), 2.56-2.51 (m, 1H), 2.18-2.02 (m, 2H), 1.78-1.62 (m, 3H).

### Intermediate 106-1:4-bromo-5-ethyl-7,7-difluoro-1-(tetrahydro-2H-pyran-2-yl)-1,5,6,7-tetrahydrocyclopenta[f]indazole (Int-106-1) & Intermediate 106-2:4-bromo-5-ethyl-7,7-difluoro-1-(tetrahydro-2H-pyran-2-yl)-1,7-dihydrocyclopenta[f]indazole (Int-106-2)

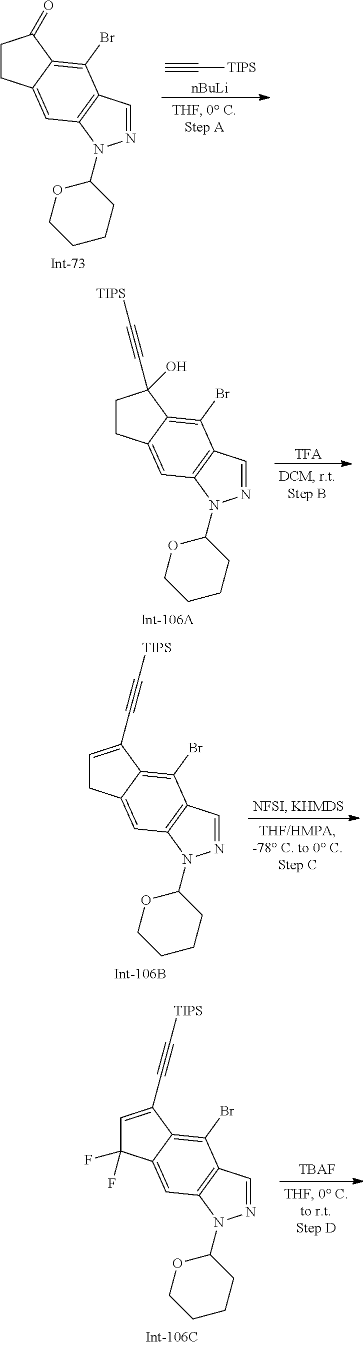

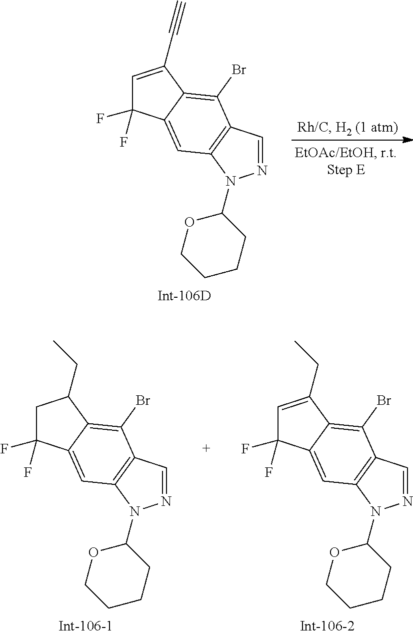

### Step A: 4-bromo-1-(tetrahydro-2H-pyran-2-yl)-5-((triisopropylsilyl)ethynyl)-1,5,6,7-tetrahydrocyclopenta[f]indazol-5-ol (Int-106A)

n-butyllithium (8.32 mL, 22.4 mmol, 2.69 M in hexane) was added to a solution of triisopropylsilylacetylene (5.02 mL, 22.4 mmol) in THF (190 mL) at 0° C. After stirring at 0° C. for 1 h, 4-bromo-1-(tetrahydro-2H-pyran-2-yl)-6,7-dihydrocyclopenta[f]indazol-5 (17)-one (Int-73) (2.50 g, 7.46 mmol) was added to the solution. After stirring at 0° C. for 2 h, the reaction mixture was diluted with EtOAc, washed with sat. aq. NH₄Cl and brine, then dried over Na₂SO₄, filtered, and evaporated. The residue was purified by silica gel chromatography (0 to 100% EtOAc in hexane) to afford 4-bromo-1-(tetrahydro-2H-pyran-2-yl)-5-((triisopropylsilyl)ethynyl)-1,5,6,7-tetrahydrocyclopenta[f]indazol-5-ol (Int-106A). MS (ESI): m/z (M+H)⁺ 517, 519.

### Step B: 4-bromo-1-(tetrahydro-2H-pyran-2-yl)-5-((triisopropylsilyl)ethynyl)-1,7-dihydrocyclopenta[f]indazole (Int-106B)

A solution of 4-bromo-1-(tetrahydro-2H-pyran-2-yl)-5-((triisopropylsilyl)ethynyl)-1,5,6,7-tetrahydrocyclopenta[f]indazol-5-ol (Int-106A) (2.50 g, 4.83 mmol) in DCM (30 mL) and TFA (0.359 mL) was stirred at room temperature for 0.5 h. After stirring, the reaction mixture was concentrated in vacuo. The residue was purified by silica gel chromatography (0 to 100% EtOAc in hexane) to provide 4-bromo-1-(tetrahydro-2H-pyran-2-yl)-5-((triisopropylsilyl)ethynyl)-1,7-dihydrocyclopenta[f]indazole (Int-106B). MS (ESI): m/z (M+H)⁺ 499, 501.

### Step C: 4-bromo-7,7-difluoro-1-(tetrahydro-2H-pyran-2-yl)-5-(triisopropylsilyl)ethynyl)-1,7-dihydrocyclopenta[f]indazole (Int-106C)

KHMDS (11.7 mL, 11.7 mmol, 1 M in THF) was added to a solution of 4-bromo-1-(tetrahydro-2H-pyran-2-yl)-5-((triisopropylsilyl)ethynyl)-1,7-dihydrocyclopenta[f]indazole (Int-106B) (1.95 g, 3.90 mmol) and N-fluorobenzenesulfonimide (3.69 g, 11.7 mmol) in THF (55.8 mL) and HMPA (11.2 mL) at −78° C. The reaction mixture was stirred at −78° C. for 0.5 h and then warmed up to 0° C. After stirring for 1 h at 0° C., the reaction mixture was diluted with EtOAc, washed with sat. aq. NH₄Cl and brine, then dried over Na₂SO₄, filtered, and evaporated. The residue was purified by silica gel chromatography (0 to 100% EtOAc in hexane) to provide 4-bromo-7,7-difluoro-1-(tetrahydro-2H-pyran-2-yl)-5-((triisopropylsilyl)ethynyl)-1,7-dihydrocyclopenta[f]indazole (Int-106C). MS (ESI): m/z (M+H)⁺ 535, 537.

### Step D: 4-bromo-5-ethynyl-7,7-difluoro-1-(tetrahydro-2H-pyran-2-yl)-1,7-dihydrocyclopenta[f]indazole (Int-106D)

TBAF (5.04 mL, 5.04 mmol, 1 M in THF) was added to a solution of 4-bromo-7,7-difluoro-1-(tetrahydro-2H-pyran-2-yl)-5-((triisopropylsilyl)ethynyl)-1,7-dihydrocyclopenta[f]indazole (Int-106C) (900 mg, 1.68 mmol) in THF (15 mL) at 0° C. The reaction was warmed to room temperature and stirred for 30 min. After 30 min, the reaction mixture was diluted with EtOAc, washed with sat. aq. NH₄Cl and brine, then dried over Na₂SO₄, filtered, and evaporated, thereby affording 4-bromo-5-ethynyl-7,7-difluoro-1-(tetrahydro-2H-pyran-2-yl)-1,7-dihydrocyclopenta[f]indazole (Int-106D), which was used without further purification. MS (ESI): m/z (M+H)⁺ 379, 381.

### Step E: 4-bromo-5-ethyl-7,7-difluoro-1-(tetrahydro-2H-pyran-2-yl)-1,5,6,7-tetrahydrocyclopenta[f]indazole (Int-106-1) & 4-bromo-5-ethyl-7,7-difluoro-1-(tetrahydro-2H-pyran-2-yl)-1,7-dihydrocyclopenta[f]indazole (Int-106-2)

Rh/C (1.1 g, 5 wt %) was added to a solution of 4-bromo-5-ethynyl-7,7-difluoro-1-(tetrahydro-2H-pyran-2-yl)-1,7-dihydrocyclopenta[f]indazole (Int-106D) (700 mg, 1.85 mmol) in EtOAc (10 mL) and EtOH (30 mL). The reaction vessel was evacuated and backfilled with H₂. After stirring at room temperature for 24 h, the reaction mixture was filtered through celite and evaporated. The residue was purified by silica gel chromatography (0 to 100% EtOAc in hexane) to afford 4-bromo-5-ethyl-7,7-difluoro-1-(tetrahydro-2H-pyran-2-yl)-1,5,6,7-tetrahydrocyclopenta[f]indazole (Int-106-1) [MS (ESI): m/z (M+H)⁺ 385, 387] and 4-bromo-5-ethyl-7,7-difluoro-1-(tetrahydro-2H-pyran-2-yl)-1,7-dihydrocyclopenta[f]indazole (Int-106-2) [MS (ESI): m/z (M+H)⁺ 383, 385].

### Intermediate 107: (5-ethyl-7,7-difluoro-1-(tetrahydro-2H-pyran-2-yl)-1,7-dihydrocyclopenta[f]indazol-4-yl)boronic acid (Int-107)

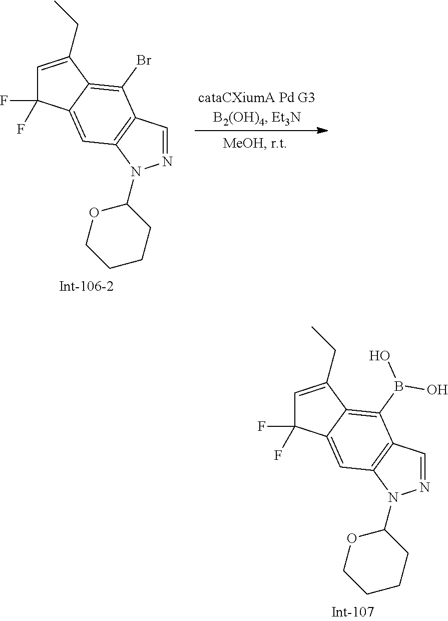 [SMILES: CCC1=CC(F)(F)c2cc3c(cnn3C3CCCCO3)c(Br)c21 ; "cataCXiumA Pd G3B2(OH)4, Et3N" ; CCC1=CC(F)(F)c2cc3c(cnn3C3CCCCO3)c(B(O)O)c21]

Tetrahydroxydiboron (9.05 mg, 0.101 mmol), cataCXium A Pd G3 (2.4 mg, 0.0046 mmol), and Et₃N (19 μL, 0.13 mmol) were added to a solution of 4-bromo-5-ethyl-7,7-difluoro-1-(tetrahydro-2H-pyran-2-yl)-1,7-dihydrocyclopenta[f]indazole (Int-106-2) (12.1 mg, 0.0363 mmol) in MeOH (0.68 mL). After stirring at room temperature for 1 h, the solution was filtered through celite and evaporated. The residue was purified by silica gel chromatography (0 to 20% EtOH in CHCl₃) to afford (5-ethyl-7,7-difluoro-1-(tetrahydro-2H-pyran-2-yl)-1,7-dihydrocyclopenta[f]indazol-4-yl)boronic acid (Int-107). MS (ESI): m/z (M+H)⁺ 349.

### Intermediate 108: (7,7-difluoro-5-methyl-1-(tetrahydro-2H-pyran-2-yl)-1,5,6,7-tetrahydrohydrocyclopenta[f]indazol-4-yl)boronic acid (Int-108)

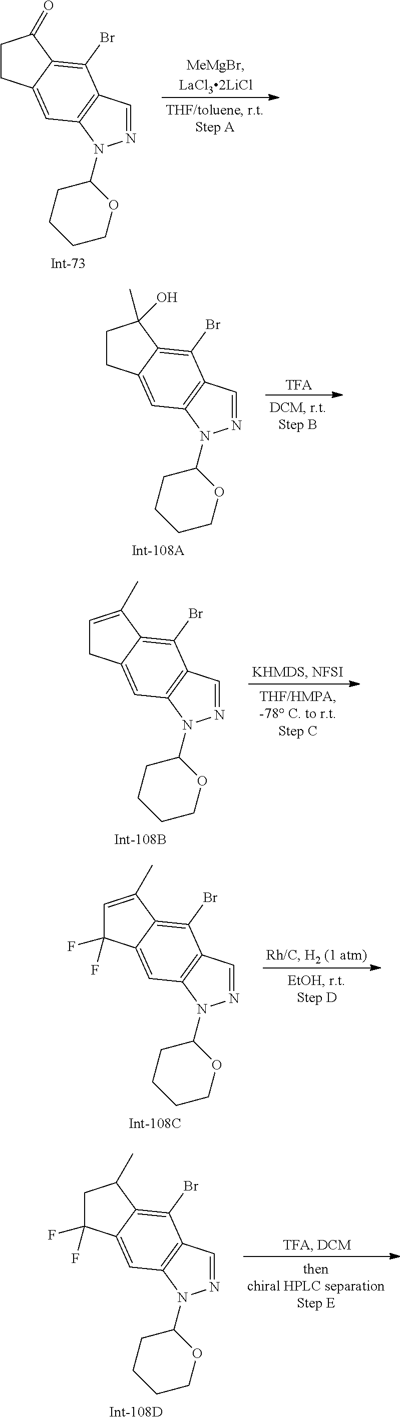

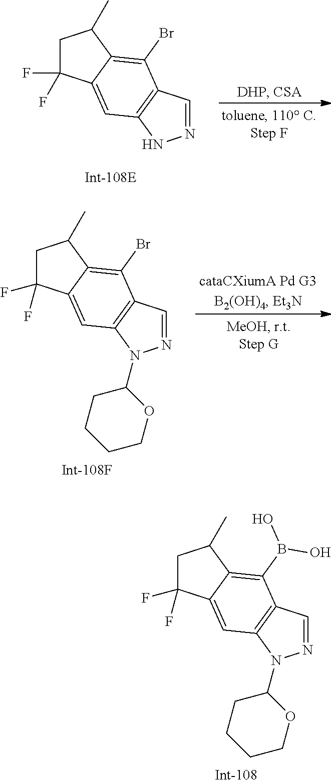

### Step A: 4-bromo-5-methyl-1-(tetrahydro-2H-pyran-2-yl)-1,5,6,7-tetrahydrocyclopenta[f]indazol-5-ol (Int-108A)

Methylmagnesium bromide (8.9 mL, 27 mmol, 3.0 M in THF) was added to a mixture of 4-bromo-1-(tetrahydro-2H-pyran-2-yl)-6,7-dihydrocyclopenta[f]indazol-5 (1H)-one (Int-73) (3.0 g, 9.0 mmol) and LaCl₃·2LiCl (3.0 mL, 1.8 mmol, 0.6 M in THF) in THF (90 mL) and toluene (90 mL) at room temperature. After stirring for 30 min, the reaction mixture was diluted with EtOAc and sat. aq. NH₄Cl. The layers were separated and the organic phase was washed with brine, dried over Na₂SO₄, filtered, and evaporated to afford 4-bromo-5-methyl-1-(tetrahydro-2H-pyran-2-yl)-1,5,6,7-tetrahydrocyclopenta[f]indazol-5-ol (Int-108A), which was used directly in the next step without further purification. MS (ESI) [M+H]⁺: m/z 351, 353.

### Step B: 4-bromo-5-methyl-1-(tetrahydro-2H-pyran-2-yl)-1,7-dihydrocyclopenta[f]indazole (Int-108B)

A solution of 4-bromo-5-methyl-1-(tetrahydro-2H-pyran-2-yl)-1,5,6,7-tetrahydrocyclopenta[f]indazol-5-ol in DCM (45 mL) and TFA (0.13 mL) was stirred at room temperature for 1 h. After stirring for 1 h, the reaction mixture was concentrated in vacuo. The residue was purified by column chromatography on silica gel (0 to 100% EtOAc in hexanes) to afford 4-bromo-5-methyl-1-(tetrahydro-2H-pyran-2-yl)-1,7-dihydrocyclopenta[f]indazole (Int-108B). MS (ESI) [M+H]⁺: m/z 333, 335.

### Step C: 4-bromo-7,7-difluoro-5-methyl-1-(tetrahydro-2H-pyran-2-yl)-1,7-dihydrocyclopenta[f]indazole (Int-108C)

KHMDS (16 mL, 16 mmol, 1 M in toluene) was added to a solution of 4-bromo-5-methyl-1-(tetrahydro-2H-pyran-2-yl)-1,7-dihydrocyclopenta[f]indazole (Int-108B) (1.5 g, 4.5 mmol) and N-fluorobenzenesulfonimide (5.0 g, 16.0 mmol) in THF (45 mL) and HMPA (4.5 mL) at −78° C. The reaction mixture was stirred at −78° C. for 5 min and then warmed to room temperature. After stirring for 1 h, the reaction was diluted with EtOAc and washed with sat. aq. NH₄Cl and brine. The combined organic layers were then dried over Na₂SO₄, filtered, and concentrated in vacuo. The residue was purified by silica gel chromatography (0 to 100% EtOAc in hexanes) to afford 4-bromo-7,7-difluoro-5-methyl-1-(tetrahydro-2H-pyran-2-yl)-1,7-dihydrocyclopenta[f]indazole (Int-108C). MS (ESD) [M+H]⁺: m/z 369, 371.

### Step D: 4-bromo-7,7-difluoro-5-methyl-1-(tetrahydro-2H-pyran-2-yl)-1,5,6,7-tetrahydrocyclopenta[f]indazole (Int-108D)

Rh/C (160 mg, 5 wt %) was added to a solution of 4-bromo-7,7-difluoro-5-methyl-1-(tetrahydro-2H-pyran-2-yl)-1,7-dihydrocyclopenta[f]indazole (Int-108C) (85 mg, 0.23 mmol) in EtOH (4.6 mL). The reaction was evacuated and backfilled with H₂. After stirring at room temperature under H₂ atmosphere for 2 h, the reaction mixture was filtered through CELITE® and concentrated in vacuo. The residue was purified by silica gel chromatography (0 to 100% EtOAc in hexanes) to afford 4-bromo-7,7-difluoro-5-methyl-1-(tetrahydro-2H-pyran-2-yl)-1,5,6,7-tetrahydrocyclopenta[f]indazole (Int-108D). MS (ESI) [M+H]⁺: m/z 371, 373.

### Step E: 4-bromo-7,7-difluoro-5-methyl-1,5,6,7-tetrahydrocyclopenta[f]indazole (Int-108E)

A solution of 4-bromo-7,7-difluoro-5-methyl-1-(tetrahydro-2H-pyran-2-yl)-1,5,6,7-tetrahydrocyclopenta[f]indazole (Int-108D) (166 mg, 0.447 mmol) in DCM (2.2 mL) and TFA (0.5 mL) was stirred at room temperature for 2 h. The reaction mixture was concentrated in vacuo and purified by silica gel chromatography (0 to 100% EtOAc in hexanes) to afford racemic 4-bromo-7,7-difluoro-5-methyl-1,5,6,7-tetrahydrocyclopenta[f]indazole. The enantiomers were separated by preparative chiral HPLC (CHIRAL CEL Oz-H, hexane/IPA=95/5) to afford 4-bromo-7,7-difluoro-5-methyl-1,5,6,7-tetrahydrocyclopenta[f]indazole (Int-108E, first eluting isomer). MS (ESI) [M+H]⁺: m/z 287, 289.

### Step F: 4-bromo-7,7-difluoro-5-methyl-1-(tetrahydro-2H-pyran-2-yl)-1,5,6,7-tetrahydrocyclopenta[f]indazole (Int-108F)

3,4-dihydro-2H-pyran (19.0 μL, 0.234 mmol) and (+)-CSA (4.80 mg, 0.021 mmol) were added to a solution of 4-bromo-7,7-difluoro-5-methyl-1,5,6,7-tetrahydrocyclopenta[f]indazole (Int-108E) (30.0 mg, 0.104 mmol) in toluene (1.0 mL). The mixture was stirred at 110° C. for 30 min. The solution was cooled to room temperature and diluted with EtOAc. The reaction was washed with brine, dried over Na₂SO₄, filtered, and concentrated in vacuo. The residue was purified by silica gel chromatography (0 to 100% EtOAc in hexanes) to afford 4-bromo-7,7-difluoro-5-methyl-1-(tetrahydro-2H-pyran-2-yl)-1,5,6,7-tetrahydrocyclopenta[f]indazole (Int-108F). MS (ESI) [M+H]⁺: m/z 371, 373.

### Step G: (7,7-difluoro-5-methyl-1-(tetrahydro-2H-pyran-2-yl)-1,5,6,7-tetrahydrohydrocyclopenta[f]indazol-4-yl)boronic acid (Int-108)

Tetrahydroxydiboron (14.4 mg, 0.161 mmol), cataXCium A Pd G3 (3.92 mg, 0.0050 mmol), and Et₃N (0.03 mL, 0.2 mmol) were added to a solution of 4-bromo-7,7-difluoro-5-methyl-1-(tetrahydro-2H-pyran-2-yl)-1,5,6,7-tetrahydrocyclopenta[f]indazole (Int-108F) (20 mg, 0.053 mmol) in MeOH (1.1 mL). After stirring at room temperature for 2 h, the solution was filtered through CELITE® and evaporated. The residue was purified by silica gel chromatography (20% EtOH in CHCl₃) to afford (7,7-difluoro-5-methyl-1-(tetrahydro-2H-pyran-2-yl)-1,5,6,7-tetrahydrohydrocyclopenta[f]indazol-4-yl)boronic acid (Int-108). MS (ESI) [M+H]⁺: m/z 337.

The compound in the table below was synthesized using a similar procedure as described in the synthesis of Int-108 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | | SFC/HPLC | |
|---|---|---|---|---|---|
| | Starting | | | Separation | |
| Ex. | Material | Structure | Compound Name | Conditions | [M + H]⁺ |
| Int-109 | 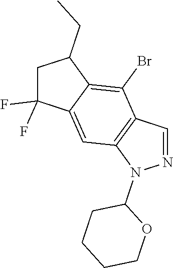 [SMILES: CCC1CC(F)(F)c2cc3c(cnn3C3CCCCO3)c(Br)c21] | 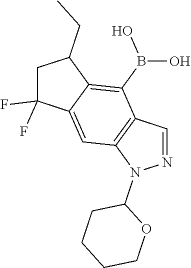 [SMILES: CCC1CC(F)(F)c2cc3c(cnn3C3CCCCO3)c(B(O)O)c21] | (5-ethyl-7,7- difluoro-1- (tetrahydro-2H- pyran-2-yl)- 1,5,6,7- tetrahydro- cyclopenta [f]indazol-4-yl) boronic acid | Step E, CHIRAL CEL Oz-H, hexane/IPA = 95/5 | 351 |
| | Int-106-1 | | | | |

### Intermediate 110: 4-bromo-5-(difluoromethylene)-1-(tetrahydro-2H-pyran-2-yl)-1,5,6,7-tetrahydrocyclopenta[f]indazole (Int-110)

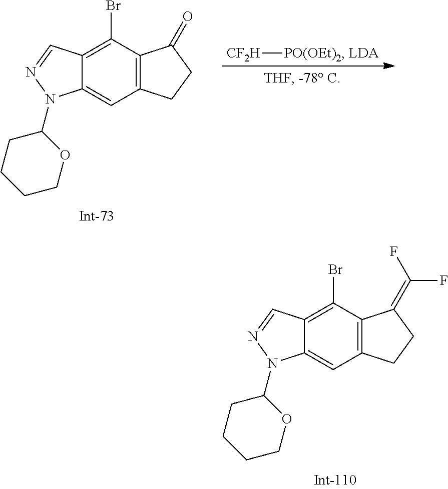

A vial was loaded with difluoromethylphosphonic acid diethyl ester (282 mg, 1.50 mmol) and THF (5 mL). The mixture was cooled to −78° C. and LDA (0.75 mL, 1.5 mmol, 2M in THF) was added dropwise. The mixture was stirred for 30 min. After 30 min, 4-bromo-1-(tetrahydro-2H-pyran-2-yl)-6,7-dihydrocyclopenta[f]indazol-5 (1H)-one (Int-73) (335 mg, 0.999 mmol) was added as a solution in THF (5 mL) in one portion. The reaction was allowed to stir at −78° C. for 1 h and then allowed to warm up to room temperature for 40 min. The reaction was quenched with water, extracted with DCM, dried over Na₂SO₄, filtered, and concentrated under reduced pressure. The crude mixture was purified by silica gel chromatography (0 to 100% EtOAc in hexanes) to provide 4-bromo-5-(difluoromethylene)-1-(tetrahydro-2H-pyran-2-yl)-1,5,6,7-tetrahydrocyclopenta[f]indazole (Int-110) MS (ESI): m/z (M+H)⁺ 369, 371.

### Intermediate 111; (5-methyl-1-(tetrahydro-2H-pyran-2-yl)-1,7-dihydrocyclopenta[f]indazol-4-yl)boronic acid (Int-111)

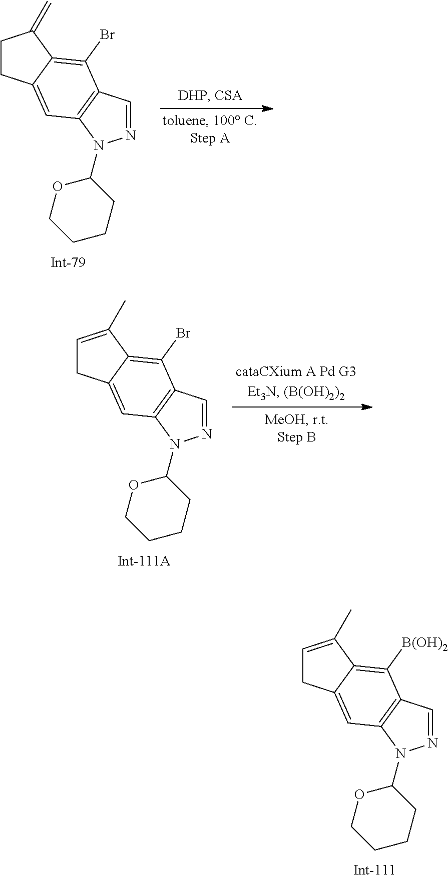 [SMILES: C=C1CCc2cc3c(cnn3C3CCCCO3)c(Br)c21 ; CC1=CCc2cc3c(cnn3C3CCCCO3)c(Br)c21 ; CC1=CCc2cc3c(cnn3C3CCCCO3)c(B(O)O)c21]

### Step A: 4-bromo-5-methyl-1-(tetrahydro-2H-pyran-2-yl)-1,7-dihydrocyclopenta[f]indazole (Int-111A)

To a stirred mixture of 4-bromo-5-methylene-1-(tetrahydro-2H-pyran-2-yl)-1,5,6,7-tetrahydrocyclopenta[f]indazole (Int-79) (95 mg, 0.29 mmol), and 3,4-dihydro-2H-pyran (0.052 mL, 0.57 mmol) in toluene (1 mL) was added camphorsulfonic acid (20 mg, 0.086 mmol) and the mixture was stirred at 100° C. for 0.5 h. After cooling the reaction to room temperature, the mixture was partitioned between EtOAc and water. The organic phase was washed with brine, dried over sodium sulfate, and concentrated. The residue was purified via silica gel chromatography (0 to 30% EtOAc in hexanes) to provide 4-bromo-5-methyl-1-(tetrahydro-2H-pyran-2-yl)-1,7-dihydrocyclopenta[f]indazole (Int-111A). MS (ESI): m/z (M+H)⁺ 333.

### Step B: (5-methyl-1-(tetrahydro-2H-pyran-2-yl)-1,7-dihydrocyclopenta[f]indazol-4-yl)boronic acid (Int-111)

To a stirred mixture of 4-bromo-5-methyl-1-(tetrahydro-2H-pyran-2-yl)-1,7-dihydrocyclopenta[f]indazole (Int-111A) (27 mg, 0.081 mmol), triethylamine (0.045 mL, 0.32 mmol), and tetrahydroxydiboron (14 mg, 0.16 mmol) in MeOH (1 mL) was added cataCXium A Pd G3 (6 mg, 0.008 mmol) and the mixture was stirred at room temperature for 2.5 h. The mixture was filtered through a pad of CELITE®, washed with MeOH, and the filtrate was concentrated. The residue was purified via silica gel chromatography (0 to 50% EtOAc in hexanes) to provide (S-methyl-1-(tetrahydro-2H-pyran-2-yl)-1,7-dihydrocyclopenta[f]indazol-4-yl)boronic acid (Int-111). MS (ESI): m/z (M+H)⁺ 299.

### Intermediate 112: ((2-fluoro-6-(methoxymethoxy)-8-(4,4,5,5-tetramethyl-1,3,2-dioxaborolan-2-yl) naphthalen-1-ylethynyl)triisopropylsilane (Int-112)

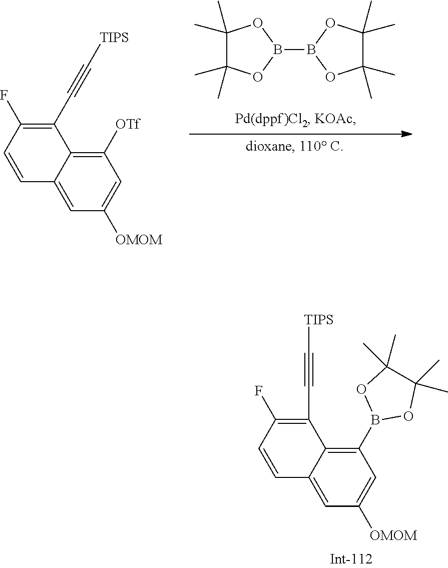

To a solution of 7-fluoro-3-(methoxymethoxy)-8-((triisopropylsilyl)ethynyl) naphthalen-1-yl trifluoromethanesulfonate (15 g, 28 mmol), bis(pinacolato)diboron (13.32 g, 52.5 mmol), [1,1′ bis(diphenylphosphino)ferrocene]dichloropalladium (II) (2.05 g, 2.81 mmol), and potassium acetate (6.88 g, 70.1 mmol) in dioxane (150 mL) was stirred at 110° C. under N₂ for 15 h.

The mixture was concentrated, the residue was purified by flash silica gel chromatography (petroleum ether) to give ((2-fluoro-6-(methoxymethoxy)-8-(4,4,5,5-tetramethyl-1,3,2-dioxaborolan-2-yl) naphthalen-1-yl) ethynyl)triisopropylsilane (Int-112). ¹H NMR (400 MHz, CDCl₃) δ 7.69-7.64 (m, 1H), 7.51 (d. J=2.5 Hz, 1H), 7.38 (d, J=2.6 Hz, 1H), 7.23 (t, J=8.8 Hz, 1H), 5.29-5.26 (m, 2H), 3.51 (s, 3H), 1.44 (s, 12H), 1.18-1.14 (m, 21H).

### Intermediate 113: 4,4,5,5-tetramethyl-2-(3-methyl-2,3-dihydro-1H-inden-4-yl)-1,3,2-dioxaborolane (Int-113)

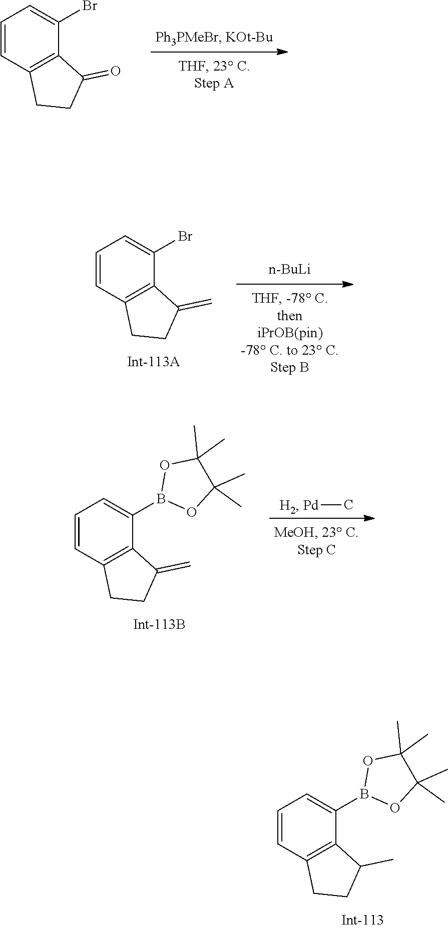

### Step A: 7-bromo-1-methylene-2,3-dihydro-1H-indene (Int-113A)

Potassium tert-butoxide (9.24 mL, 9.24 mmol, 1 M solution in THF) was added at room temperature to a stirred suspension of methyltriphenylphosphonium bromide (3.30 g, 9.24 mmol) in THF (71.1 mL). After stirring for 45 min, 7-bromo-2,3-dihydro-1H-inden-1-one (1.50 g, 7.11 mmol) was added. After an additional 2.5 h, the mixture was diluted with half-saturated aq. ammonium chloride solution (50 mL). The mixture was extracted with hexanes (3×60 mL). The combined extracts were washed with brine (30 mL), dried over anhydrous sodium sulfate, filtered, and concentrated. The residue was purified by flash-column chromatography (0 to 10% EtOAc in hexanes) to provide 7-bromo-1-methylene-2,3-dihydro-1H-indene (Int-113A). ¹H NMR (500 MHz, CDCl₃) δ 7.39 (d, J=7.8 Hz, 1H), 7.20 (d, J=7.1 Hz, 1H), 7.02 (t, J=7.6 Hz, 1H), 6.24 (s, 1H), 5.22 (s, 1H), 2.95 (t, J=6.9 Hz, 2H), 2.84-2.83 (m, 2H).

### Step B: 4,4,5,5-tetramethyl-2-(3-methylene-2,3-dihydro-1H-inden-4-yl)-1,3,2-dioxaborolane (Int-113B)

n-butyllithium (2.53 mL, 6.31 mmol, 2.50 M in hexanes) was added dropwise to a solution of 7-bromo-1-methylene-2,3-dihydro-1H-indene (Int-113A) (1.10 g, 5.26 mmol) in THF (35.1 mL) at −78° C. The mixture was stirred at this temperature for 1 h. Next, 2-isopropoxy-4,4,5,5-tetramethyl-1,3,2-dioxaborolane (1.40 mL, 6.84 mmol) was added dropwise. The mixture was warmed to room temperature. After stirring at room temperature for 2 h, the mixture was diluted with half-saturated aqueous ammonium chloride solution and diethyl ether. The layers were shaken, then separated. The aqueous phase was extracted with diethyl ether, and the combined extracts were dried over anhydrous sodium sulfate. The filtrate was concentrated and the residue was purified by flash-column chromatography (0 to 30% EtOAc in hexanes) to provide 4,4,5,5-tetramethyl-2-(3-methylene-2,3-dihydro-1H-inden-4-yl)-1,3,2-dioxaborolane (Int-113B). MS (ESI): m/z (M+H)⁺ 257.

### Step C: 4,4,5,5-tetramethyl-2-(3-methyl-2,3-dihydro-1H-inden-4-yl)-1,3,2-dioxaborolane (Int-113)

4,4,5,5-tetramethyl-2-(3-methylene-2,3-dihydro-1H-inden-4-yl)-1,3,2-dioxaborolane (Int-113B) (0.30 g, 1.2 mmol) was dissolved in MeOH (12 mL) and palladium on carbon (60 mg, 10% w/w) was added. The mixture was stirred at room temperature under 1 atm of hydrogen gas for 4 h before the mixture was filtered through a pad of celite. The filtrate was concentrated to provide pure 4,4,5,5-tetramethyl-2-(3-methyl-2,3-dihydro-1H-inden-4-yl)-1,3,2-dioxaborolane (Int-113). ¹H NMR (500 MHz, CDCl₃) δ 7.60 (d, J=6.8 Hz, 1H), 7.29 (d, J=6.9 Hz, 1H), 7.13 (t, J=7.1 Hz, 1H), 3.67-3.65 (m, 1H), 3.02 (dt, J=17.2, 9.6 Hz, 1H), 2.78 (dd, J=15.0, 9.2 Hz, 1H), 2.21 (m, 1H), 1.76 (t, J=9.8 Hz, 1H), 1.34 (s, 12 Hz), 1.17 (d, J=6.7 Hz, 3H).

The compound in the table below was synthesized using a similar procedure as described in the synthesis of Int-113 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-114 | 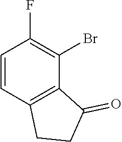 [SMILES: O=C1CCc2ccc(F)c(Br)c21] | 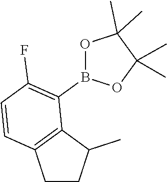 [SMILES: CC1CCc2ccc(F)c(B3OC(C)(C)C(C)(C)O3)c21] | 2-(5-fluoro-3- methyl-2,3- dihydro-1H- inden-4-yl)- 4,4,5,5- tetramethyl- 1,3,2- dioxaborolane | 277 |

### Intermediate 115: 4,4,5,5-tetramethyl-2-(1,1a,6,6a-tetrahydrocyclopropa[a]inden-5-yl)-1,3,2-dioxaborolane (Int-115)

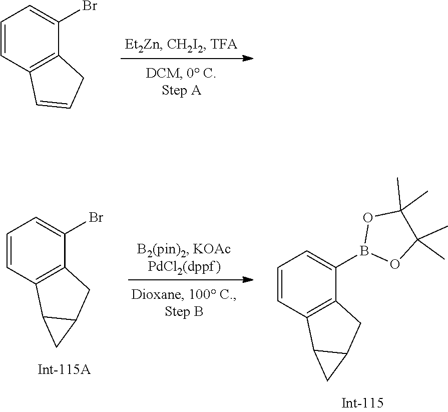 [SMILES: Brc1cccc2c1CC=C2 ; Brc1cccc2c1CC1CC21 ; CC1(C)OB(c2cccc3c2CC2CC32)OC1(C)C]

### Step A: 5-bromo-1,1a,6,6a-tetrahydrocyclopropa[a]indene (Int-115A)

Diethylzinc (4.00 mL, 4.00 mmol, 1 M in hexanes) was diluted in DCM (4 mL) and chilled to 0° C. A solution of TFA (0.310 mL, 4.02 mmol) in DCM (2.00 mL) was added dropwise and was stirred for 20 minutes at 0° C. A solution of diiodomethane (0.320 mL, 3.97 mmol) in DCM (2.00 mL) was added and the resulting mixture was allowed to stir for an additional 20 minutes at 0° C. A solution of 7-bromo-1H-indene (420 mg, 2.15 mmol) in DCM (2.00 mL) was added and the resulting mixture was allowed to stir for 2 hours. The reaction was quenched with sat. aq. NaHCO₃ (5 mL) and stirred for 5 minutes. Sat. aq. NH₄Cl was added and the product was extracted with DCM before drying over sodium sulfate and concentrating. The residue was purified by column chromatography (hexanes) to afford 5-bromo-1,1a,6,6a-tetrahydrocyclopropa[a]indene (Int-115A). ¹H NMR (500 MHz, CDCl₃) δ 7.27-7.21 (m, 2H), 7.00 (t, J=7.5 Hz, 1H), 3.15 (dd, J=17.5, 6.6 Hz, 1H), 2.99 (d, J=17.5 Hz, 1H), 2.45 (s, 1H), 1.90 (s, 1H), 1.11 (q, J=7.8 Hz, 1H), 0.14 (s, 1H).

### Step B: 4,4,5,5-tetramethyl-2-(1,1a,6,6a-tetrahydrocyclopropa[a]inden-5-yl)-1,3,2-dioxaborolane (Int-115)

5-bromo-1,1a,6,6a-tetrahydrocyclopropa[a]indene (372 mg, 1.78 mmol) (Int-115A), bis(pinacolato)diboron (904 mg, 3.56 mmol), potassium acetate (524 mg, 5.34 mmol), and PdCl₂ (dppf) dichloromethane adduct (145 mg, 0.178 mmol) were added to a vial. The vial was sealed and its contents were placed under an inert atmosphere by performing 3 vacuum/nitrogen cycles. Dioxane (10 mL) was added through the septum and the resulting mixture was allowed to stir for 5 hours at 100° C. The reaction mixture was cooled, filtered through a pad of CELITE®, and concentrated under reduced pressure. The residue was purified by column chromatography on silica (0 to 100% EtOAc in hexane) to afford 4,4,5,5-tetramethyl-2-(1,1a,6,6a-tetrahydrocyclopropa[a]inden-5-yl)-1,3,2-dioxaborolane (Int-115). MS (ESI) [M+H]⁺: m/z 257.

### Intermediate 116: (3-chloro-2-(trimethylsilyl)phenyl)boronic acid (Int-116)

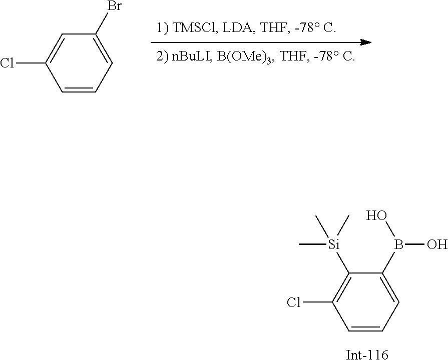

To a mixture of 1-bromo-3-chlorobenzene (500 mg, 2.61 mmol) and TMSCl (0.40 mL, 3.1 mmol) in THF (8.7 mL) was added LDA (1.6 mL, 3.1 mmol, 2.0 M in THF) at −78° C. After the mixture was stirred at −78° C. for 10 min, the mixture was quenched by the addition of water. The reaction mixture was extracted with EtOAc (3×50 mL). The combined organic layers were washed with brine (20 mL), dried over Na₂SO₄, filtered, and concentrated under reduced pressure. The residue was purified via silica gel chromatography (100% hexanes) to give (2-bromo-6-chlorophenyl)trimethylsilane, which was used directly in the next step without further purification.

To a mixture of (2-bromo-6-chlorophenyl)trimethylsilane in THF (1.3 mL) was added nBuLi (0.11 mL, 0.22 mmol, 2.0 M in hexanes) at −78° C. After the mixture was stirred at −78° C. for 45 min, trimethylborate (0.033 mL, 0.30 mmol) was added and the reaction mixture was stirred at −78° C. for 90 min. The mixture was quenched by the addition of H₂SO₄ (1.3 mL, 2.0 M in H₂O). The reaction mixture was extracted with EtOAc (3×50 mL). The combined organic layers were washed with brine (20 mL), dried over Na₂SO₄, filtered, and concentrated under reduced pressure. The residue was purified via silica gel chromatography (5 to 100% EtOAc in hexane) to give (3-chloro-2-(trimethylsilyl)phenyl)boronic acid (Int-116). ¹H NMR (400 MHz, CDCl₃) δ 7.59 (dd, J=0.8, 7.2 Hz, 1H), 7.42 (dd, J=1.2, 8.0 Hz, 1H), 7.34-7.26 (m, 1H), 4.62 (s, 2H), 0.44 (s, 9H).

### Intermediate 117:4-bromo-6-fluoro-N,N-bis(4-methoxybenzyl)-5-((triisopropylsilyl)ethynyl) quinolin-2-amine (Int-117)

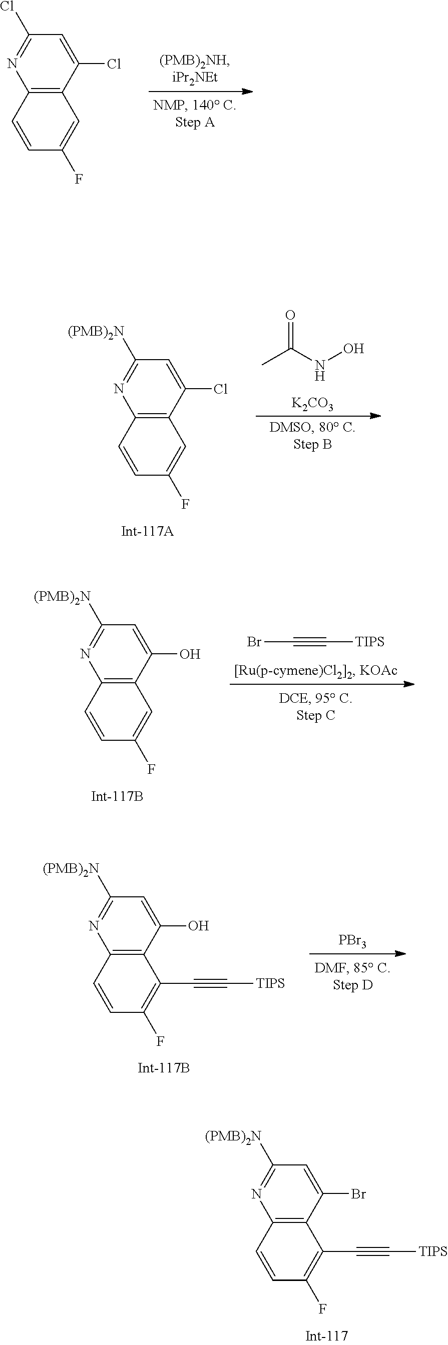

### Step A: 4-chloro-6-fluoro-N,N-bis(4-methoxybenzyl) quinolin-2-amine (Int-117A)

A solution of 2,4-dichloro-6-fluoroquinoline (5.0 g, 23 mmol), bis(4-methoxybenzyl)amine (11.9 g, 46.3 mmol), and N,N-diisopropylethylamine (8.08 mL, 46.3 mmol) in NMP (46 mL) was allowed to stir at 140° C. for 18 h. After 18 h, the reaction was cooled to room temperature and water was added followed by extraction with EtOAc. The combined organic layers were dried over magnesium sulfate, filtered, and concentrated to give a crude product. The crude residue was purified via silica gel chromatography (0 to 10% EtOAc in hexanes) to give 4-chloro-6-fluoro-N,N-bis(4-methoxybenzyl) quinolin-2-amine (Int-117A). MS (ESI): m/z (M+H)⁺ 437.

### Step B: 2-(bis(4-methoxybenzyl)amino)-6-fluoroquinolin-4-ol (Int-117B)

To a slurry solution of 4-chloro-6-fluoro-N,N-bis(4-methoxybenzyl) quinolin-2-amine (Int-117A) (3.8 g, 8.70 mmol) in DMSO (17 ml) was added acetohydroxamic acid (9.79 g, 130 mmol) and potassium carbonate (18 g, 130 mmol). The resultant mixture was allowed to stir at 80° C. for 18 h. After 18 h, the reaction was cooled to r.t., and the reaction mixture was slowly added to water. The resultant precipitate was filtered to give 2-(bis(4-methoxybenzyl)amino)-6-fluoroquinolin-4-ol (Int-117B). MS (ESI): m/z (M+H)⁺ 419.

### Step C: 2-(bis(4-methoxybenzyl)amino)-6-fluoro-5-(triisopropylsilyl)ethynyl)quinolin-4-ol (Int-117C)

To a solution of 2-(bis(4-methoxybenzyl)amino)-6-fluoroquinolin-4-ol (Int-117B) (3.7 g, 8.8 mmol), dichloro(p-cymene) ruthenium (II) dimer (1.354 g, 2.210 mmol), and potassium acetate (1.736 g, 17.7 mmol) in DCE (75 ml) was added (bromoethynyl)triisopropylsilane (4.62 g, 17.68 mmol). The resultant reaction mixture was allowed to stir at 95° C. for 18 h. After 18 h, the reaction was cooled to r.t., and water was added followed by extraction with DCM. The combined organic layers were dried over magnesium sulfate, filtered, and concentrated to give crude product. The crude residue was purified via silica gel chromatography (100% DCM) to give 2-(bis(4-methoxybenzyl)amino)-6-fluoro-5-((triisopropylsilyl)ethynyl) quinolin-4-ol (Int-117C). MS (ESI): m/z (M+H)⁺ 599.

### Step D: 4-bromo-6-fluoro-N,N-bis(4-methoxybenzyl)-5-((triisopropylsilyl)ethynyl) quinolin-2-amine (Int-117)

To a solution of 2-(bis(4-methoxybenzyl)amino)-6-fluoro-5-((triisopropylsilyl)ethynyl) quinolin-4-ol (Int-117C) (1.4 g, 2.3 mmol) in DMF (25 ml) was added phosphorus tribromide (0.330 mL, 3.51 mmol). The resulting reaction mixture was allowed to stir at 85° C. for 18 h. After 18 h, the reaction was cooled to r.t., and 1N aq. NaOH was added until pH >7. The reaction was extracted with EtOAc, then the combined organic layers were washed with 10% aq. LiCl, dried over magnesium sulfate, filtered, and concentrated to give crude product. The crude residue was purified via silica gel chromatography (100% DCM) to give 4-bromo-6-fluoro-N,N-bis(4-methoxybenzyl)-5-((triisopropylsilyl)ethynyl) quinolin-2-amine (Int-117). MS (ESI): m/z (M+H)⁺ 661.

### Intermediate 118-1: (S,E)-7a-(tert-butyldiphenylsilyl)oxy)methyl)-2-(fluoromethylene) hexahydro-1H-pyrrolizine (Int-118-1) & Intermediate 118-2: (S,Z)-7a-(((tert-butyldiphenylsilyl)oxy)methyl)-2-(fluoromethylene) hexahydro-1H-pyrrolizine (Int-118-2)

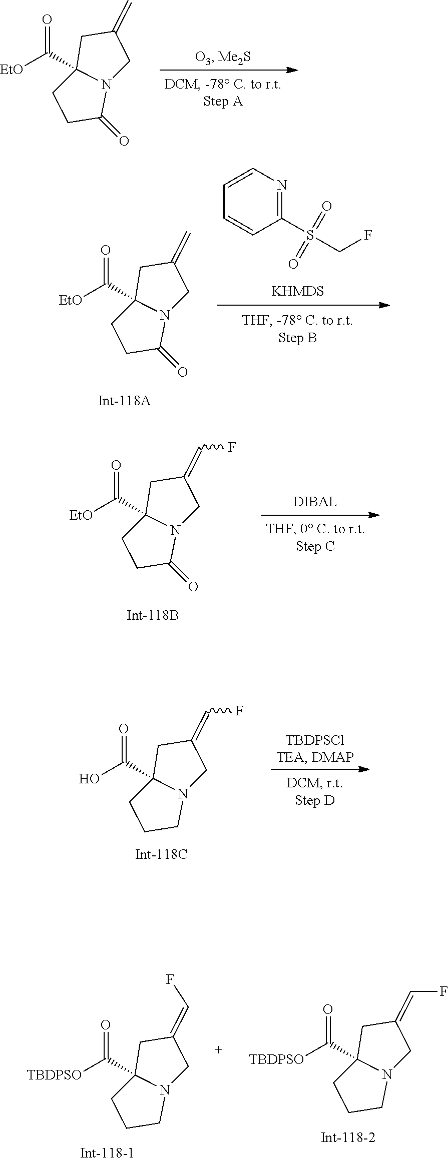

### Step A: Ethyl(S)-2,5-dioxotetrahydro-1H-pyrrolizine-7a(5H)-carboxylate (Int-118A)

A solution of ethyl(S)-2-methylene-5-oxotetrahydro-1H-pyrrolizine-7a(5H)-carboxylate (1.0 g, 4.8 mmol) in DCM (15 mL) was bubbled with 03 at −78° C. until the mixture turned blue. Dimethylsulfide (0.594 g, 9.56 mmol) was added to the mixture and the reaction was stirred at 25° C. for 16 h. The reaction mixture was washed with brine (20 mL), dried over sodium sulfate, filtered, and concentrated under reduced pressure to give the crude product. The crude product was purified by flash silica gel chromatography (0 to 50% EtOAc in petroleum ether) to give ethyl(S)-2,5-dioxotetrahydro-1H-pyrrolizine-7a(5H)-carboxylate (Int-118A). MS (ESI) [M+H]⁺: m/z 212.

### Step B: Ethyl(S)-2-(fluoromethylene)-5-oxotetrahydro-1H-pyrrolizine-7a(5H)-carboxylate (Int-118B)

To a solution of fluoromethyl 2-pyridyl sulfone (684 mg, 3.91 mmol) in THF (10 mL) was added KHMDS (4.51 mL, 4.51 mmol, 1 M in THF) at −78° C. under N₂. After 30 min, ethyl(S)-2,5-dioxotetrahydro-1H-pyrrolizine-7a(5H)-carboxylate (Int-118A) (750 mg, 3.55 mmol) in THF (8 mL) was added slowly at −78° C. and the mixture was stirred for 3 h. The reaction was warmed to room temperature and stirred for 1 h. The reaction was quenched with sat. aq. NH₄Cl (1 mL), and 3 M HCl (2 mL). The mixture was extracted with EtOAc (3×20 mL). The organic layer was dried over sodium sulfate, filtered, and the solvent was evaporated under reduced pressure to give the crude product. The crude product was purified by flash silica gel chromatography (0 to 30% EtOAc in petroleum ether) to give ethyl(S)-2-(fluoromethylene)-5-oxotetrahydro-1H-pyrrolizine-7a(5H)-carboxylate (Int-118B). MS (ESI) [M+H]⁺: m/z 228.

### Step C: (S)-(2-(fluoromethylene)tetrahydro-1H-pyrrolizin-7a(5H)-yl) methanol (Int-118C)

To a solution of ethyl(S)-2-(fluoromethylene)-5-oxotetrahydro-1H-pyrrolizine-7a(5H)-carboxylate (Int-118B) (200 mg, 0.880 mmol) in THF (4 mL) at 0° C. was added DIBAL (8.80 mL, 8.80 mmol, 1 M in toluene) at 0° C. and the mixture was stirred for 1 h. The reaction was warmed to room temperature and stirred for 0.5 h. The reaction mixture was quenched with solid Na₂SO₄·10H₂O. The mixture was filtered and the filtrate was concentrated under reduced pressure. The crude residue was purified by preparative TLC (DCM/MeOH=10/1) to give(S)-(2-(fluoromethylene)tetrahydro-1H-pyrrolizin-7a(5H)-yl) methanol (Int-118C). MS (ESI) [M+H]⁺: m/z 172.

### Step D: (S,E)-7a-(((tert-butyldiphenylsilyl)oxy)methyl)-2-(fluoromethylene)hexahydro-1H-pyrrolizine (Int-118-1) & (S,Z)-7a-(((tert-butyldiphenylsilyl)oxy)methyl)-2-(fluoromethylene)hexahydro-1H-pyrrolizine (Int-118-2)

To a mixture of(S)-(2-(fluoromethylene)tetrahydro-1H-pyrrolizin-7a(5H)-yl) methanol (Int-118C) (120 mg, 0.701 mmol) in DCM (5 mL) was added DMAP (8.6 mg, 0.070 mmol), Et₃N (0.195 mL, 1.40 mmol), and tert-butylchlorodiphenylsilane (289 mg, 1.05 mmol) at 0° C. under N₂, and the mixture was stirred at 25° C. for 16 h. The mixture was diluted with water (5 mL) and extracted with DCM (3×20 mL). The combined organic layers were washed with water (10 mL) and brine (10 mL), dried over sodium sulfate, filtered, and concentrated under reduced pressure. The crude reaction mixture was purified by flash silica gel chromatography (0 to 50% EtOAc in petroleum ether) to give (S,E)-7a-(((tert-butyldiphenylsilyl)oxy)methyl)-2-(fluoromethylene) hexahydro-1H-pyrrolizine (Int-118-1, the first peak eluting isomer, MS (ESI) [M+H]⁺: m/z 410) and (S,Z)-7a-(((tert-butyldiphenylsilyl)oxy)methyl)-2-(fluoromethylene) hexahydro-1H-pyrrolizine (Int-118-2, the second peak eluting isomer, MS (ESI) [M+H]⁺: m/z 410).

### Intermediate 119: (S,Z)-(2-(fluoromethylene)tetrahydro-1H-pyrrolizin-7a(5H)-yl) methanol (Int-119)

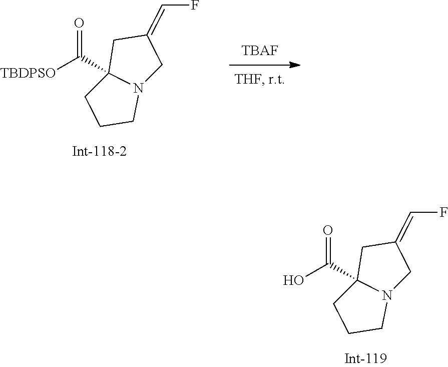

To a mixture of (S,Z)-7a-(((tert-butyldiphenylsilyl)oxy)methyl)-2-(fluoromethylene) hexahydro-1H-pyrrolizine (Int-118-2) (91 mg, 0.22 mmol) in THF (1 mL) was added TBAF (0.444 mL, 0.444 mmol, 1 M in THF) at 25° C. under N₂, and the mixture was stirred at 25° C. for 16 h. The mixture was diluted with water (10 mL) and extracted with EtOAc (3×20 mL). The organic layer was dried over sodium sulfate, filtered, and the solvent was evaporated under reduced pressure to give the crude product, which was purified by preparative TLC (DCM/MeOH=10:1) to give (S,Z)-(2-(fluoromethylene)tetrahydro-1H-pyrrolizin-7a(5H)-yl) methanol (Int-119). MS (ESI) [M+H]⁺: m/z 172.

The compound in the table below was synthesized using a similar procedure as described in the synthesis of Int-119 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-120 | 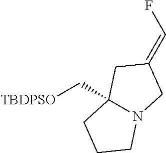 | 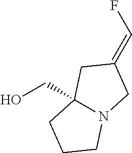 [SMILES: OC[C@@]12CCCN1C/C(=C/F)C2] | (S,E)-(2- (fluoromethylene) tetrahydro-1H- pyrrolizin-7a(5H)- yl)methanol | 172 |
| | Int-118-1 | | | |

### Intermediate 121-1: Ethyl(1S,7a′S)-2,2-difluoro-5′-oxodihydro-1′H,3′H-spiro[cyclopropane-1,2′-pyrrolizine]-7a′ (5′H)-carboxylate (Int-121-1) & Intermediate 121-2: Ethyl(1R,7a′S)-2,2-difluoro-5′-oxodihydro-1′H,3′-spiro[cyclopropane-1,2′-pyrrolizine]-7a (5′H)-carboxylate (Int-121-2)

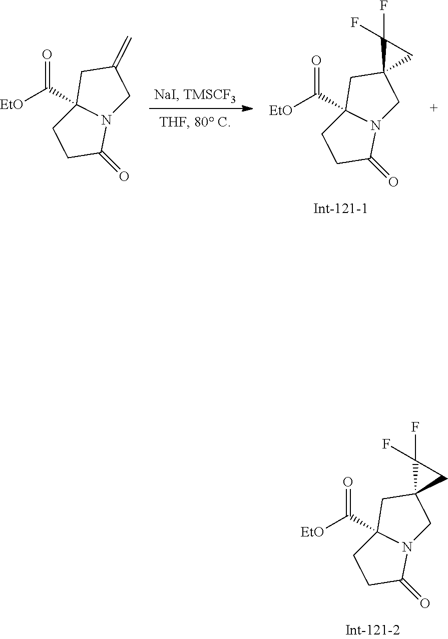 [SMILES: C=C1CN2C(=O)CC[C@@]2(C(=O)OCC)C1 ; CCOC(=O)[C@@]12CCC(=O)N1C[C@@]1(CC1(F)F)C2 ; CCOC(=O)[C@@]12CCC(=O)N1C[C@]1(CC1(F)F)C2]

TMSCF₃ (951 mg, 6.69 mmol) was added to a solution of ethyl(S)-2-methylene-5-oxotetrahydro-1H-pyrrolizine-7a(5H)-carboxylate (400 mg, 1.91 mmol) and sodium iodide (143 mg, 0,956 mmol) in THF (2 mL). The resulting solution was stirred at 80° C. for 16 h. The mixture was cooled, filtered, and the filtrate was evaporated under reduced pressure to give the crude product. The crude product was purified by flash silica gel chromatography (0 to 100% EtOAc in petroleum ether) to give ethyl(1S,7a′S)-2,2-difluoro-5′-oxodihydro-1′H,3′H-spiro[cyclopropane-1,2′-pyrrolizine]-7a (5′H)-carboxylate (Int-121-1, the first eluting isomer) and ethyl(1R,7a′S)-2,2-difluoro-5′-oxodihydro-1′H,3′H-spiro[cyclopropane-1,2′-pyrrolizinc]-7a′ (5′H)-carboxylate (Int-121-2, the second eluting isomer), MS (ESI) [M+H]⁺: m/z 260.

### Intermediate 122-1: ((1S,7a′S)-2,2-difluorodihydro-1′H,3′H-spiro[cyclopropane-1,2′-pyrrolizin]-7a′ (5′H)-yl) methanol (Int-122-1)

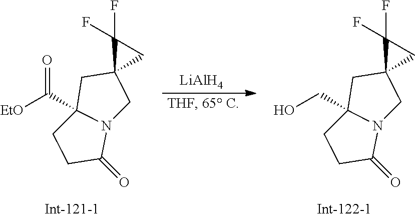 [SMILES: CCOC(=O)[C@@]12CCC(=O)N1C[C@@]1(CC1(F)F)C2 ; "LiAlH4" ; "THF, 65° C." ; O=C1CC[C@@]2(CO)C[C@@]3(CN12)CC3(F)F]

To a solution of ethyl(1S,7a′S)-2,2-difluoro-5′-oxodihydro-1′H,3′H-spiro[cyclopropane-1,2′-pyrrolizine]-7a′ (5′H)-carboxylate (180 mg, 0.694 mmol) (Int-121-1) in THF (5 mL) was added LiAlH₄ (79 mg, 2.1 mmol) at 25° C. The suspension was stirred at 65° C. for 1 h. The reaction was cooled to room temperature and the mixture was quenched with solid Na₂SO₄·10H₂O. After filtration, the filtrate was evaporated under reduced pressure to give ((1S,7a′S)-2,2-difluorodihydro-1′H,3′H-spiro[cyclopropane-1,2′-pyrrolizin]-7a′ (5′H)-yl) methanol (Int-122-1). MS (ESI) [M+H]⁺: m/z 204.

The compound in the table below was synthesized using a similar procedure as described in the synthesis of Int-122-1 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-122-2 | 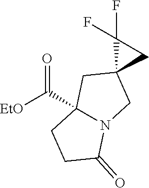 [SMILES: CCOC(=O)[C@@]12CCC(=O)N1C[C@]1(CC1(F)F)C2] | 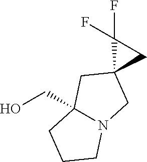 [SMILES: OC[C@@]12CCCN1C[C@]1(CC1(F)F)C2] | ((1R,7a′S)-2,2- difluorodihydro-1′H,3′H- spiro[cyclopropane-1,2′- pyrrolizin]-7a′(5′H)- yl)methanol | 204 |
| | Int-121-2 | | | |

### Intermediate 123: (R)-(1-(2-oxa-6-azaspiro[3,3]heptan-6-yl)methyl)-2,2-difluorocyclopropyl) methanol (Int-123)

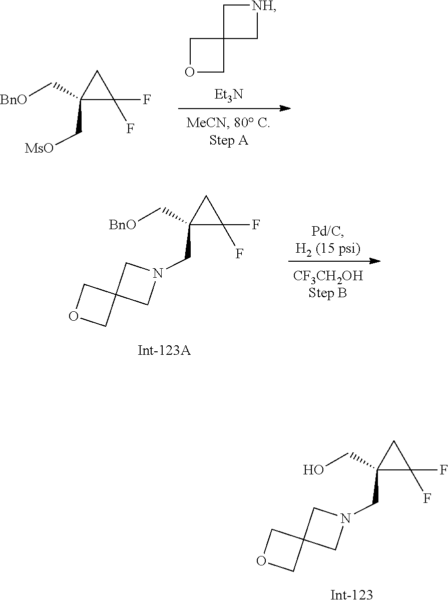

### Step A: (R)-6-((1-((benzyloxy)methyl)-2,2-difluorocyclopropyl methyl)-2-oxa-6-azaspiro[3,3]heptane (Int-123A)

To a solution of(S)-(1-((benzyloxy)methyl)-2,2-difluorocyclopropyl)methyl methanesulfonate (464 mg, 1.51 mmol) in MeCN (10 mL) was added 2-oxa-6-azaspiro[3,3]heptane (125 mg, 1.26 mmol) and triethylamine (0.701 mL, 5.04 mmol). The mixture was stirred at 80° C. for 3 h. The mixture was concentrated under reduced pressure.

The crude product was purified by flash silica gel chromatography (0 to 10% MeOH in DCM) to give (R)-6-((1-((benzyloxy)methyl)-2,2-difluorocyclopropyl)methyl)-2-oxa-6-azaspiro[3,3]heptane (Int-123A), MS (ESD) [M+H]⁺: m/z 310.

### Step B: (R)-(1-((2-oxa-6-azaspiro[3,3]heptan-6-yl)methyl)-2,2-difluorocyclopropyl) methanol (Int-123)

To a solution of (R)-6-((1-((benzyloxy)methyl)-2,2-difluorocyclopropyl)methyl)-2-oxa-6-azaspiro[3,3]heptane (Int-123A) (190 mg, 0.614 mmol) in CF₃CH₂OH (5 mL) was was added Pd/C (65 mg, 0.061 mmol, 10 wt %). The resulting suspension was stirred at 25° C. for 16 h under H₂ (15 psi). The reaction mixture was filtered and the filter cake was washed with CH₃CH₂OH. The filtrate was concentrated in vacuo to give (R)-(1-((2-oxa-6-azaspiro[3,3]heptan-6-yl)methyl)-2,2-difluorocyclopropyl) methanol (Int-123), which was used directly in the next step without further purification. MS (ESI) [M+H]⁺: m/z 220.

### Intermediate 124: ((R)-2,2-difluoro-1-(((1R,5S,6R)-6-fluoro-3-azabicyclo[3.1.0]hexan-3-yl)methyl)cyclopropyl) methanol (Int-124)

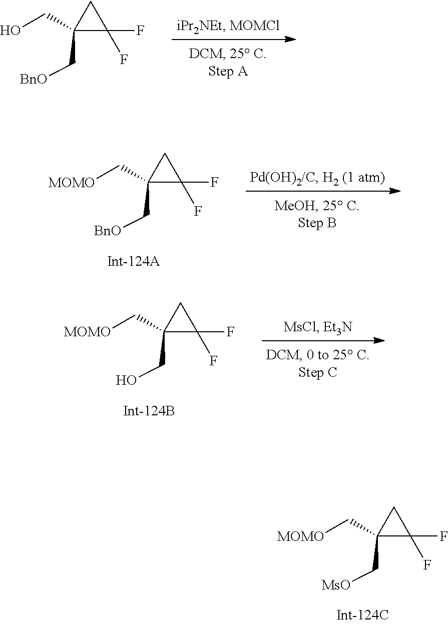

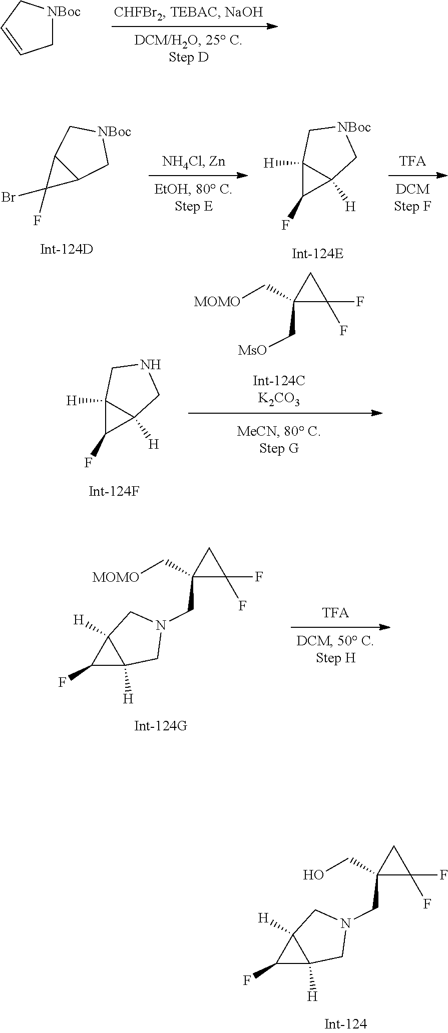

### Step A: (R)-(((2,2-difluoro-1-((methoxymethoxy)methyl)cyclopropyl)methoxy)methyl)benzene (Int-124A)

(S)-(1-((benzyloxy)methyl)-2,2-difluorocyclopropyl) methanol (50.0 g, 219 mmol) was dissolved in DCM (500 mL) at room temperature. N,N-diisopropylethylamine (77.0 mL, 438 mmol) and chloromethyl methyl ether (25.0 mL, 329 mmol) were added via syringe and the mixture was stirred under N₂ overnight. NH₄Cl (10% aq.) and MTBE (200 mL ca.) were added and the mixture was stirred for 15 min. The layers were separated and the organic layer was washed with sat. aq. NaHCO₃ (3×200 mL). The combined organic layers were dried over Na₂SO₄, filtered, and concentrated to provide (R)-(((2,2-difluoro-1-((methoxymethoxy)methyl)cyclopropyl)methoxy)methyl)benzene (Int-124A), which was used directly in the next step without further purification. [M+H]⁺: m/z 273.

### Step B: (R)-(2,2-difluoro-1-((methoxymethoxy)methyl)cyclopropyl) methanol (Int-124B)

(R)-(((2,2-difluoro-1-((methoxymethoxy)methyl)cyclopropyl)methoxy)methyl)benzene (Int-124A) was added to a 3-necked round bottom flask with a stir bar and dissolved in MeOH (400 mL). Pd(OH)₂ on carbon (3.0 g, 10 wt %) was added to the reaction mixture and the headspace was evacuated and backfilled with N₂ (3×). The headspace was placed under vacuum and the flask was equipped with a hydrogen balloon. The mixture was stirred at room temperature for 72 b. The reaction mixture was filtered through a pad of CELITE® and concentrated in vacuo to afford (R)-(2,2-difluoro-1-((methoxymethoxy)methyl)cyclopropyl) methanol (Int-124B). MS (ESI) [M+H]⁺: m/z 183.

### Step C: (S)-(2,2-difluoro-1-((methoxymethoxy)methyl)cyclopropyl methyl metbanesulfonate (Int-124C)

To a solution of (R)-(2,2-difluoro-1-((methoxymethoxy)methyl)cyclopropyl) methanol (Int-124B) (1.0 g, 5.5 mmol) in DCM (10 mL) was added triethylamine (3.06 mL, 22.0 mmol) and MsCl (1.3 mL, 16.9 mmol) at 0° C. under N₂. The reaction mixture was warmed to room temperature and stirred at 25° C. for 2 h. The reaction was quenched with saturated aqueous NaHCO₃ (20 mL) and extracted with DCM (3×15 mL). The organic layers were dried over sodium sulfate, filtered, and concentrated in vacuo. The crude product was purified by flash silica gel chromatography (30% EtOAc in petroleum ether) to give(S)-(2,2-difluoro-1-((methoxymethoxy)methyl)cyclopropyl)methyl methanesulfonate (Int-124C). ¹H NMR (400 MHz, CDCl₃) δ 4.66 (s, 2H), 4.48-4.33 (m, 2H), 3.77-3.54 (m, 2H), 3.45-3.33 (m, 3H), 3.06 (s, 3H), 1.60-1.53 (m, 1H), 1.46 (ddd, J=11.2, 8.4, 5.8 Hz, 1H).

### Step D: Tert-butyl 6-bromo-6-fluoro-3-azabicyclo[3.1.0]hexane-3-carboxylate (Int-124D)

To a solution of tert-butyl 2,5-dihydro-1H-pyrrole-1-carboxylate (2.0 g, 12 mmol) in DCM (20 mL) was added dibromofluoromethane (11.3 g, 59.1 mmol) and benzyltricthylammonium chloride (0.269 g, 1,182 mmol). The reaction was cooled to 0° C. and NaOH (10 mL, 11.82 mmol, 50 wt % in H₂O) was added dropwise. The mixture was warmed to room temperature and stirred for 16 h. The mixture was diluted with water (50 mL) and extracted with EtOAc (3×50 mL). The combined organic layers were dried over sodium sulfate, filtered, and the solvent was evaporated under reduced pressure to give the crude product. The crude product was purified by flash silica gel chromatography (17% EtOAc in petroleum ether) to give tert-butyl 6-bromo-6-fluoro-3-azabicyclo[3.1.0]hexane-3-carboxylate (Int-124D). MS (ESI) [M+H]⁺: m/z 280, 282.

### Step E: Tert-butyl(1R,5S,6r)-6-fluoro-3-azabicyclo[3.1.0]hexane-3-carboxylate (Int-124E)

To a solution of tert-butyl 6-bromo-6-fluoro-3-azabicyclo[3.1.0]hexane-3-carboxylate (Int-124D) (1.2 g, 4.3 mmol) in EtOH (15 mL) was added NH₄Cl (0.687 g, 12.9 mmol) and zinc powder (0.560 g, 8.57 mmol) at 20° C. The reaction was stirred at 80° C. for 16 h. The mixture was cooled to room temperature, diluted with H₂O (20 mL), and extracted with EtOAc (3×30 mL). The combined organics were dried over sodium sulfate, filtered, and the solvent was evaporated under reduced pressure. The crude product was purified by flash silica gel chromatography (15% EtOAc in petroleum ether) to give tert-butyl(1R,5S,6r)-6-fluoro-3-azabicyclo[3.1.0]hexane-3-carboxylate (Int-124E, the first eluting peak). MS (ESI) [M+H]⁺: m/z 202.

### Step F: (1R,5S,6r)-6-fluoro-3-azabicyclo[3.1.0]hexane (Int-124F)

To a solution of TFA (1.0 mL, 13 mmol) in DCM (3 mL) was added tert-butyl(1R,5S,6r)-6-fluoro-3-azabicyclo[3.1.0]hexane-3-carboxylate (Int-124E) (450 mg, 2.24 mmol) at 25° C. The mixture was stirred for 20 min. The reaction was concentrated in vacuo to give (1R,5S,6r)-6-fluoro-3-azabicyclo[3.1.0]hexane (Int-124F). MS (ESI) [M+H]⁺: m/z 102.

### Step G: (1R,5S,6R)-3-(((R)-2,2-difluoro-1-(methoxymethoxy)methyl)cyclopropyl)methyl)-6-fluoro-3-azabicyclo[3.1.0]hexane (Int-124G)

To a solution of (1R,5S,6r)-6-fluoro-3-azabicyclo[3.1.0]hexane (Int-124F) (170 mg, 1.68 mmol) in MeCN (3 mL) was added (S)-(2,2-difluoro-1-((methoxymethoxy)methyl)cyclopropyl)methyl methanesulfonate (Int-124C) (525 mg, 2.02 mmol) and K₂CO₃ (2.32 g, 16.8 mmol). The mixture was stirred at 80° C. for 3 h under N₂. The reaction was concentrated in vacuo and the residue was purified by flash silica gel chromatography (0 to 10% EtOAc in petroleum ether) to give (1R,5S,6R)-3-(((R)-2,2-difluoro-1-((methoxymethoxy)methyl)cyclopropyl)methyl)-6-fluoro-3-azabicyclo[3.1.0]hexane (Int-124G). MS (ESI) [M+H]⁺: m/z 266.

### Step H: ((R)-22-difluoro-1-(((1R,5S,6R)-6-fluoro-3-azabicyclo[3.1.0]hexan-3-yl)methyl)cyclopropyl) methanol (Int-124)

To a solution of TFA (0.25 mL, 3.2 mmol) in DCM (0.25 mL) was added (1R,5S,6R)-3-(((R)-2,2-difluoro-1-((methoxymethoxy)methyl)cyclopropyl)methyl)-6-fluoro-3-azabicyclo[3.1.0]hexane (Int-124G) (35 mg, 0.13 mmol) at room temperature under N₂. The mixture was stirred at 50° C. for 3 h. The reaction was concentrated in vacuo to give ((R)-2,2-difluoro-1-(((1R,5S,6R)-6-fluoro-3-azabicyclo[3.1.0]hexan-3-yl)methyl)cyclopropyl) methanol (Int-124). MS (ESI) [M+H]⁺: m/z 222.

### Intermediate 125: (1R,5R,6S)-2-methyl-2-azabicyclo[3.2.0]heptan-6-ol (Int-125)

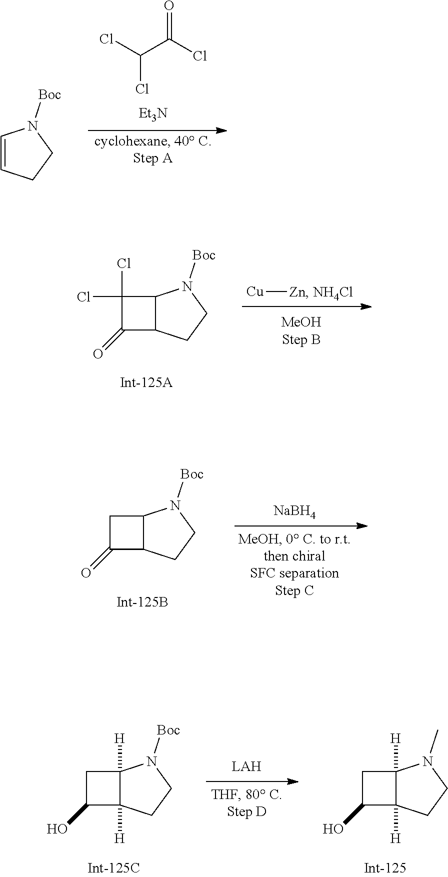

### Step A: Tert-butyl 7,7-dichloro-6-oxo-2-azabicyclo[3.2.0]heptane-2-carboxylate (Int-125A)

To a solution of tert-butyl 2,3-dihydro-1H-pyrrole-1-carboxylate (4.5 g, 27 mmol) in cyclohexane (70 mL) was added triethylamine (7.39 mL, 53.2 mmol) and 2,2-dichloroacetyl chloride (3.84 mL, 39.9 mmol) at 25° C. under N₂. The reaction was stirred at 40° C. for 16 h. The reaction mixture was partitioned into H₂O (80 mL) and EtOAc (90 mL). The layers were shaken and separated and the aqueous phase was extracted with EtOAc (2×90 mL). The combined organic layers were dried over sodium sulfate, filtered, and concentrated in vacuo. The crude product was purified by flash silica gel chromatography (8% EtOAc in petroleum ether) to give tert-butyl 7,7-dichloro-6-oxo-2-azabicyclo[3.2.0]heptane-2-carboxylate (Int-125A). ¹H NMR (400 MHz, CDCl₃) δ: 4.73-4.94 (m, 1H), 4.19-4.29 (m, 1H), 3.79-3.95 (m, 1H), 3.24-3.38 (m, 1H), 2.22-2.33 (m, 1H), 2.05-2.12 (m, 1H), 1.49-1.54 (s, 9H).

### Step B: Tert-butyl 6-oxo-2-azabicyclo[3.2.0]heptane-2-carboxylate (Int-125B)

To a solution of tert-butyl 7,7-dichloro-6-oxo-2-azabicyclo[3.2.0]heptane-2-carboxylate (3.0 g, 11 mmol) (Int-125A) in MeOH (110 mL) was added zinc-copper couple (7.59 g, 58.9 mmol) and NH₄Cl (1.72 g, 32.1 mmol) at 25° C. under N₂ and the reaction was stirred for 2 h. The reaction mixture was poured into H₂O (100 mL) and extracted with EtOAc (3×140 mL). The organic layer was dried over sodium sulfate, filtered, and concentrated in vacuo to give tert-butyl 6-oxo-2-azabicyclo[3.2.0]heptane-2-carboxylate (Int-125B). MS (ESI) [M+H-tBu]⁺: m/z 156.

### Step C: Tert-butyl(1R,5R,6S)-6-hydroxy-2-azabicyclo[3.2.0]heptane-2-carboxylate (Int-125C)

A 100 mL round-bottom flask was charged with tert-butyl 6-oxo-2-azabicyclo[3.2.0]heptane-2-carboxylate (Int-125B) (1.8 g, 8.52 mmol), fitted with a septum, and placed under argon atmosphere. MeOH (20 mL) was added and the reaction was cooled to 0° C. NaBH₄ (0.645 g, 17.0 mmol) was added in portions and the mixture was stirred at room temperature for 1 h. The mixture was quenched with sat. aq. NH₄Cl (15 mL) under nitrogen atmosphere at 0° C., diluted with H₂O (20 mL), and extracted with EtOAc (3×30 mL). The combined organic layers were washed with brine (30 mL), dried over sodium sulfate, filtered, and concentrated in vacuo. The crude product was purified by flash silica gel chromatography (10% EtOAc:EtOH (3:1) in petroleum ether). The racemic tert-butyl 6-hydroxy-2-azabicyclo[3.2.0]heptane-2-carboxylate was separated by preparative chiral SFC (Column K, 35% EtOH w/0.1% NH₄OH) to provide tert-butyl(1R,5R,6S)-6-hydroxy-2-azabicyclo[3.2.0]heptane-2-carboxylate (Int-125C) (the first eluting isomer), but the desired compound eluted with impurities. The resultant material was purified a second time by preparative chiral SFC (Column K, 35% EtOH w/0.1% NH₄OH) to provide tert-butyl(1R,5R,6S)-6-hydroxy-2-azabicyclo[3.2.0]heptane-2-carboxylate (Int-125C). MS (ESI) [M+H-tBu]⁺: m/z 158.

### Step D: (1R,5R,6S)-2-methyl-2-azabicyclo[3.2.0]heptan-6-ol (Int-125)

To a solution of tert-butyl(1R,5R,6S)-6-hydroxy-2-azabicyclo[3.2.0]heptane-2-carboxylate (Int-125C) (200 mg, 0.938 mmol) in THF (3 mL) was added lithium aluminum hydride (142 mg, 3.75 mmol) at 0° C. The reaction suspension was stirred at 80° C. for 5 h. Aq. Na₂SO₄·10H₂O was added slowly dropwise to quench the reaction mixture at 0° C. The mixture was stirred at 25° C. for 2 h then filtered. The filter cake was washed with DCM (3×20 mL). The filtrate was concentrated in vacuo to give the crude product. The crude product was purified by preparative TLC (SiO₂; DCM:MeOH=20:1) to afford (1R,5R,6S)-2-methyl-2-azabicyclo[3.2.0]heptan-6-ol (Int-125). ¹H NMR (400 MHz, CDCl₃) δ: 4.27 (qd, J=7.4, 0.7 Hz, 1H), 3.36 (q, J=5.5 Hz, 1H), 3.06-3.15 (m, 1H), 2.93 (ddd, J=9.6, 7.9, 1.5 Hz, 1H), 2.62 (td, J=10.0, 6.9 Hz, 1H), 2.28 (s, 3H), 2.23-2.27 (m, 2H), 2.02-2.10 (m, 1H), 1.78-1.91 (m, 2H).

### Intermediate 126: ((4aS,7aR)-1-methyloctahydro-4aH-cyclopenta[b]pyridin-4a-yl) methanol (Int-126)

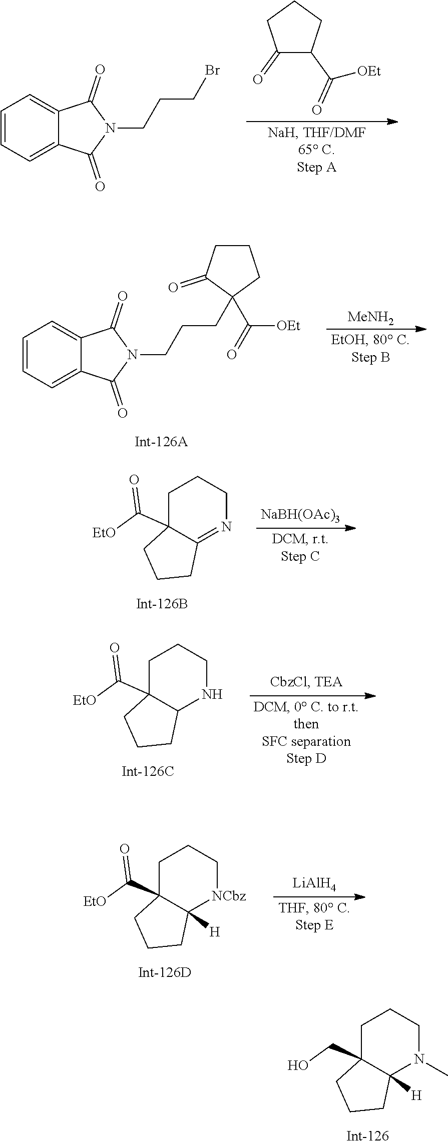

### Step A: Ethyl 1-(3-(1,3-dioxoisoindolin-2-yl) propyl)-2-oxocyclopentane-1-carboxylate (Int-126A)

Sodium hydride (4.61 g, 115 mmol, 60 wt %) was suspended in a mixture of dry THF and DMF (1:1; 500 mL). Ethyl 2-oxocyclopentane-1-carboxylate (14 mL, 96 mmol) was dissolved in dry THF (50 mL) and added dropwise at 0° C. The resulting solution was stirred at 0° C. for 10 min. Then 2-(3-bromopropyl) isoindoline-1,3-dione (28.3 g, 106 mmol) in dry THF (80 mL) was added slowly at 0° C. The reaction mixture was fitted with a reflux condenser and heated at 65° C. for 16 h. The reaction mixture was cooled to room temperature and adjusted to pH ˜7 with 1 M HCl. The reaction mixture was partitioned into water (300 mL) and EtOAc (350 mL). The layers were shaken and separated and the aqueous phase was extracted with EtOAc (2×350 mL). The combined organic layers were dried over sodium sulfate, filtered, and the solvent was evaporated under reduced pressure. The crude product was purified by flash silica gel chromatography (25% EtOAc in petroleum ether) to give ethyl 1-(3-(1,3-dioxoisoindolin-2-yl) propyl)-2-oxocyclopentane-1-carboxylate (Int-126A). MS (ESI) [M+H]⁺: m/z 344.

### Step B: Ethyl 2,3,4,5,6,7-hexahydro-4aH-cyclopenta[b]pyridine-4a-carboxylate (Int-126B)

To a solution of ethyl 1-(3-(1,3-dioxoisoindolin-2-yl) propyl)-2-oxocyclopentane-1-carboxylate (Int-126A) (14.5 g, 42.2 mmol) in EtOH (130 mL) was added methylamine (15.9 g, 169 mmol, 33 wt % in ethanol) dropwise at room temperature. The reaction mixture was fitted with a reflux condenser and heated to 80° C. for 3 h. The reaction mixture was cooled to room temperature, filtered, and the filter cake was washed with EtOAc (150 mL). The filtrate was concentrated in vacuo to afford the crude product. The crude product was purified by flash silica gel chromatography (20% MeOH in DCM) to give ethyl 2,3,4,5,6,7-hexahydro-4aH-cyclopenta[b]pyridine-4a-carboxylate (Int-126B). MS (ESI) [M+H]⁺: m/z 196.

### Step C: Ethyl octahydro-4aH-cyclopenta[b]pyridine-4a-carboxylate (Int-126C)

To a stirred solution of ethyl 2,3,4,5,6,7-hexahydro-4aH-cyclopenta[b]pyridine-4a-carboxylate (Int-126B) (6.2 g, 32 mmol) and 4 Å molecular sieves (6 g) in DCM (80 mL) was added NaBH(OAc); (13.5 g, 63.5 mmol) at room temperature and the reaction mixture was stirred for 20 h. The mixture was basified with sat. aq. NaHCO₃ (pH=7-8), filtered, and the filtrate was extracted with DCM (3×100 mL). The organic layer was dried over sodium sulfate, filtered, and concentrated in vacuo to provide ethyl octahydro-4aH-cyclopenta[b]pyridine-4a-carboxylate (Int-126C). MS (ESI) [M+H]⁺: m/z 198.

### Step D: 1-benzyl 4a-ethyl(4aS,7aR)-hexahydro-1H-cyclopenta[b]pyridine-1,4a(2H)-dicarboxylate (Int-126D)

To a solution of ethyl octahydro-4aH-cyclopenta[b]pyridine-4a-carboxylate (Int-126C) (5.0 g, 25.3 mmol) in DCM (100 mL) was added triethylamine (17.6 mL, 127 mmol) and benzyl chloroformate (8.92 mL, 63.4 mmol) at 0° C. under N₂. The mixture was warmed up to room temperature and basified with sat. aq. NaHCO₃ (pH=7-8), filtered, and the filtrate was extracted with DCM (3×130 mL). The organic layer was dried over sodium sulfate, filtered, and concentrated in vacuo. The crude product was purified by flash silica gel chromatography (15% EtOAc in petroleum ether) to provide racemic 1-benzyl 4a-ethyl hexahydro-1H-cyclopenta[b]pyridine-1,4a (2H)-dicarboxylate. MS (ESI) [M+H]⁺: m/z 332. The racemic 1-benzyl 4a-ethyl hexahydro-1H-cyclopenta[b]pyridine-1,4a (2H)-dicarboxylate was separated by preparative SFC (Column L, 20% MeOH w/0.1% NH₄OH) to provide 1-benzyl 4a-ethyl hexahydro-1H-cyclopenta[b]pyridine-1,4a (2H)-dicarboxylate (mixture of the first and second eluting isomer, four total isomers). 1-benzyl 4a-ethyl hexahydro-1H-cyclopenta[b]pyridine-1,4a (2)-dicarboxylate was separated by preparative SFC (Column M, 20% MeOH w/0.1% NH₄OH) to provide 1-benzyl 4a-ethyl(4aS,7aR)-hexahydro-1H-cyclopenta[b]pyridine-1,4a (2H)-dicarboxylate (Int-126D, the first eluting isomer, two total isomers). MS (ESI) [M+H]⁺: m/z 332.

### Step E: ((4aS 7aR)-1-methyloctahydro-4aH-cyclopenta[b]pyridin-4a-yl) methanol (Int-126)

To a solution of 1-benzyl 4a-ethyl(4aS,7aR)-hexahydro-1H-cyclopenta[6]pyridine-1,4a (2H)-dicarboxylate (Int-126D) (300 mg, 0.905 mmol) in THF (3 mL) was added lithium aluminum hydride (137 mg, 3.62 mmol) at 0° C. The reaction suspension was heated to 80° C. and stirred for 1.5 h. The reaction mixture was cooled to 0° C. and solid Na₂SO₄·10H₂O was added in portions to quench the reaction mixture. The mixture was warmed to 25° C., stirred for 2 h, and filtered. The filter cake was washed with DCM (3×20 mL). The filtrate was concentrated in vacuo to give the crude product. The crude product was purified by preparative TLC (SiO₂; DCM: 7N NH₃ in MeOH=15:1) to afford ((4aS,7aR)-1-methyloctahydro-4aH-cyclopenta[b]pyridin-4a-yl) methanol (Int-126). ¹H NMR (400 MHz, CDCl₃) δ: 3.70-3.79 (m, 1H), 3.56 (d, J=10.8 Hz, 1H), 3.07 (br t, J=7.8 Hz, 1H), 2.62-2.53 (m, 2H), 2.38 (s, 3H), 2.02-1.57 (m, 7H), 1.55-1.45 (m, 3H).

### Intermediate 127: (4-(dimethylamino)-2-oxabicyclo[3.1.0]hexan-5-yl) methanol (Int-127)

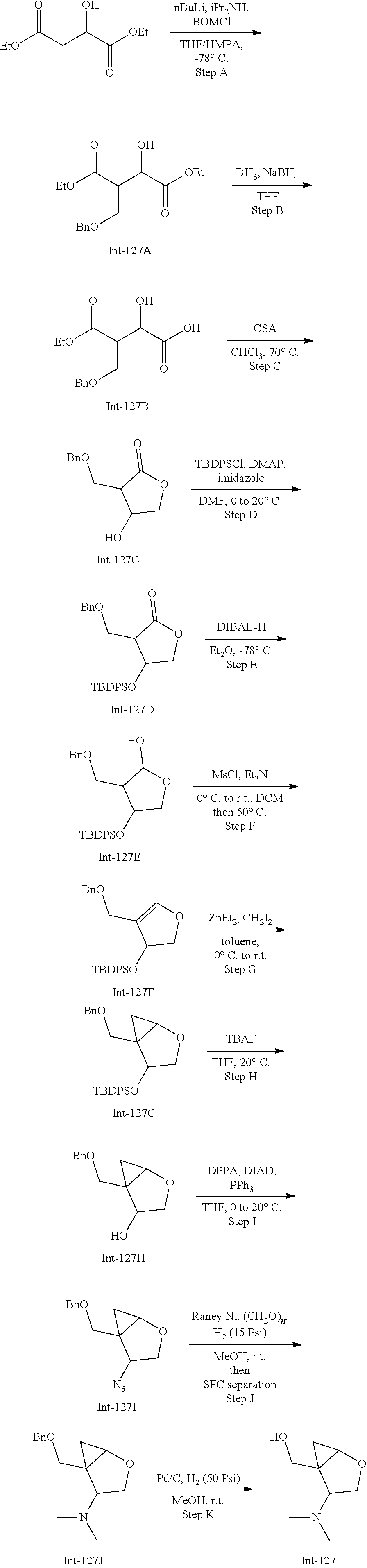

### Step A: Diethyl 2-((benzyloxy)methyl)-3-hydroxysuccinate (Int-127A)

nBuLi (95.0 mL, 238 mmol, 2.5 M in THF) was added dropwise to a solution of diisopropylamine (43.0 mL, 108 mmol) in THF (400 mL) at 0° C. The resulting solution was stirred at 0° C. for 10 min and cooled to −78° C. A solution of diethyl 2-hydroxysuccinate (20.6 g, 108 mmol) in THF (200 mL) was added dropwise. The solution was stirred at −78° C. for 30 min and then stirred at −20° C. for 1 h. After cooling back to −78° C., a solution of ((chloromethoxy)methyl)benzene (18.6 g, 119 mmol) in HMPA (80 mL) was added dropwise. The reaction was stirred at −78° C. for 3 h. Glacial acetic acid (300 mL) and EtOAc (400 mL) were added and the mixture was poured into H₂O (50 mL) and extracted with EtOAc (2×500 mL). The combined organic layers were washed with 1 M HCl (2×250 mL), saturated NaHCO₃ (50 mL), and brine (50 mL), dried over sodium sulfate, filtered, and the solvent was evaporated under reduced pressure. The residue was purified by flash silica gel chromatography (0 to 20% EtOAc in petroleum ether) to give diethyl 2-((benzyloxy)methyl)-3-hydroxysuccinate (Int-127A), ¹H NMR (400 MHz, CDCl₃) δ: 7.39-7.30 (m, 5H), 4.67-4.54 (m, 3H), 4.32-4.21 (m, 2H), 4.19-4.09 (m, 2H), 3.96-3.89 (m, 1H), 3.88-3.77 (m, 1H), 3.30 (ddd, J=8.7, 5.7, 2.8 Hz, 1H), 1.30 (t, J=7.1 Hz, 3H), 1.24 (t. J=7.2 Hz, 3H).

### Step B: Ethyl 2-((benzyloxy)methyl)-3,4-dihydroxybutanoate (Int-127B)

Borane (100 mL, 100 mmol, 1 M in THF) was added dropwise to a solution of diethyl 2-((benzyloxy)methyl)-3-hydroxysuccinate (Int-127A) (31.0 g, 100 mmol) in THF (60 mL) at 0° C. The solution was warmed to 20° C. and stirred for 30 min. The reaction was cooled back to 0° C. and NaBH₄ (0.38 g, 10 mmol) was added. The reaction was cooled back to 0° C. and additional NaBH₄ (0.378 g, 9.99 mmol) was added. The resulting mixture was warmed to 20° C. and stirred for 12 h. The mixture was diluted with MeOH (200 mL), filtered, and the solvent was evaporated under reduced pressure to give the crude product. The residue was purified by flash silica gel chromatography (0 to 10% EtOAc petroleum ether) to give ethyl 2-((benzyloxy)methyl)-3,4-dihydroxybutanoate (Int-127B). ¹H NMR (400 MHz, CDCl₃) δ: 7.33-7.10 (m, 5H), 4.58-4.38 (m, 2H), 4.27-3.94 (m, 3H), 3.92-3.48 (m, 4H), 2.81 (q, J=5.8 Hz, 1H), 1.26-1.09 (m, 3H).

### Step C: 3-((benzyloxy)methyl)-4-hydroxydihydrofuran-2 (3H)-one (Int-127C)

To a solution of ethyl 2-((benzyloxy)methyl)-3,4-dihydroxybutanoate (Int-127B) (13.8 g, 51.4 mmol) in CHCl₃ (2.96 L) was added (+)-camphor-10-sulfonic acid (1.43 g, 6.17 mmol) at 20° C. The mixture was heated to 70° C. and stirred for 24 h. The mixture was evaporated under reduced pressure to give the crude product. The residue was purified by flash silica gel chromatography (0 to 10% EtOAc petroleum ether) to give 3-((benzyloxy)methyl)-4-hydroxydihydrofuran-2 (3H)-one (Int-127C). ¹H NMR (400 MHz, CDCl₃) δ: 7.36-7.14 (m, 5H), 4.68-4.53 (m, 1H), 4.53-4.38 (m, 2H), 4.30-4.04 (m, 2H), 4.02-3.71 (m, 2H), 2.84-2.65 (m, 1H).

### Step D: 3-((benzyloxy)methyl)-4-((tert-butyldiphenylsilyl)oxy)dihydrofuran-2 (3H)-one (Int-127D)

To a solution of 3-((benzyloxy)methyl)-4-hydroxydihydrofuran-2 (3H)-one (Int-127C) (9.0 g, 41 mmol) in DMF (40 mL) was added imidazole (11.0 g, 162 mmol), DMAP (990 mg, 8.1 mmol) and tert-butylchlorodiphenylsilane (23.38 g, 85.06 mmol) at 0° C. under N₂. The mixture was warmed to 20° C. and stirred for 24 h. The mixture was diluted with water (40 mL) and extracted with EtOAc (3×50 mL). The combined organic layers were washed with brine (3×20 mL), dried over sodium sulfate, filtered, and the solvent was evaporated under reduced pressure. The residue was purified by flash silica gel chromatography (0 to 10% EtOAc petroleum ether) to give 3-((benzyloxy)methyl)-4-(tert-butyldiphenylsilyl)dihydrofuran-2 (3H)-one (Int-127D). ¹H NMR (400 MHz, CDCl₃) δ: 7.72-7.51 (m, 4H), 7.43-7.06 (m, 11H), 4.65 (t, J=3.8 Hz, 1H), 4.57-4.39 (m, 2H), 4.09-3.68 (m, 4H), 2.76 (ddd, J=9.1, 5.1, 3.9 Hz, 1H), 1.00 (s, 9H).

### Step E: 3-((benzyloxy)methyl)-4-((tert-butyldiphenylsilyl)oxy)tetrahydrofuran-2-ol (Int-127E)

To a solution of 3-((benzyloxy)methyl)-4-((tert-butyldiphenylsilyl)oxy)dihydrofuran-2 (3H)-one (Int-127D) (12.5 g, 27.1 mmol) in Et₂O (140 mL) was added DIBAL-H (40.7 mL, 40.7 mmol, 1 M in toluene) at −78° C. under N₂. The mixture was stirred at −78° C. for 5 h. Na₂SO₄·10H₂O (30 g) was added then water (20 mL). The mixture was filtered and the solvent was evaporated under reduced pressure. The crude product was purified by flash silica gel chromatography (0 to 30% EtOAc in petroleum ether) to give 3-((benzyloxy)methyl)-4-((tert-butyldiphenylsilyl)oxy)tetrahydrofuran-2-ol (Int-127E). ¹H NMR (400 MHz, CDCl₃) δ: 7.59-7.75 (m, 4H), 7.29-7.54 (m, 11H), 5.28-5.57 (m, 1H), 4.33-4.70 (m, 3H), 3.77-3.93 (m, 3H), 3.55-3.70 (m, 1H), 2.18-2.38 (m, 1H), 1.05-1.18 (m, 9H).

### Step F: ((4-(benzyloxy)methyl)-2,3-dihydrofuran-3-yl)oxy)(tert-butyl)diphenylsilane (Int-127F)

To a solution of 3-((benzyloxy)methyl)-4-((tert-butyldiphenylsilyl)oxy)tetrahydrofuran-2-ol (Int-127E) (12.0 g, 25.9 mmol) in DCM (120 mL) was added triethylamine (21.6 mL, 155 mmol), and methanesulfonyl chloride (11.9 g, 104 mmol) at 0° C. under N₂. The mixture was stirred at 0° C. for 2.5 h. The mixture was then warmed to 50° C. and stirred for 12 h. The mixture was diluted with sat. aq. NaHCO₃ (60 mL) and extracted with DCM (3×200 mL). The combined organic layers were washed with brine (3×100 mL), dried over sodium sulfate, filtered, and the solvent was evaporated under reduced pressure. The residue was purified by flash silica gel chromatography (0 to 25% EtOAc in petroleum ether) to give ((4-((benzyloxy)methyl)-2,3-dihydrofuran-3-yl)oxy) (tert-butyl)diphenylsilane (Int-127F). ¹H NMR (400 MHz, CDCl₃) δ: 7.57-7.67 (m, 4H), 7.20-7.38 (m, 11H), 6.27-6.54 (m, 1H), 5.08 (dd, J=7.0, 2.4 Hz, 1H), 4.29-4.37 (m, 2H), 3.95-4.13 (m, 3H), 3.86 (dd, J=10.8, 7.2 Hz, 1H), 1.00 (s, 9H).

### Step G: ((5-((benzyloxy)methyl)-2-oxabicyclo[3.1.0]hexan-4-yl)oxy)(tert-butyl)diphenylsilane (Int-127G)

To a solution of ((4-((benzyloxy)methyl)-2,3-dihydrofuran-3-yl)oxy) (tert-butyl)diphenylsilane (Int-127F) (9.3 g, 21 mmol) in anhydrous toluene (90 mL) was added CH₂I₂ (8.4 mL, 105 mmol) at 0° C. under N₂. The mixture was stirred at 0° C. for 30 min. ZnEt₂ (115 mL, 115 mmol, 1 M solution in hexane) was added dropwise at 0° C. The reaction mixture was warmed to 20° C. and stirred for 12 h. The mixture was quenched with sat. aq. NH₄Cl (120 mL), extracted with EtOAc (3×100 mL), dried over sodium sulfate, filtered, and the solvent was evaporated under reduced pressure to give the crude product. The residue was purified by preparative TLC (SiO₂, petroleum ether:ethyl acetate=10:1) to give ((5-((benzyloxy)methyl)-2-oxabicyclo[3.1.0]hexan-4-yl)oxy) (tert-butyl)diphenylsilane (Int-127G). ¹H NMR (400 MHz, CDCl₃) δ: 7.57-7.67 (m, 4H), 7.22-7.39 (m, 11H), 4.47-4.54 (m, 2H), 4.26-4.39 (m, 2H), 3.88 (dd, J=5.5, 1.9 Hz, 1H), 3.52 (d, J=10.8 Hz, 1H), 3.12-3.30 (m, 1H), 3.06 (d, J=9.8 Hz, 1H), 1.02 (s, 9H), 0.79-0.81 (m, 1H), 0.53-0.65 (m, 1H).

### Step H: 5-((benzyloxy)methyl)-2-oxabicyclo[3.1.0]hexan-4-ol (Int-127H)

To a solution of ((5-((benzyloxy)methyl)-2-oxabicyclo[3.1.0]hexan-4-yl)oxy) (tert-butyl)diphenylsilane (Int-127G) (7.5 g, 16 mmol) in THF (40 mL) was added TBAF (40 mL, 40.0 mmol, 1M in THF) at 20° C. under N₂. The mixture was stirred at 20° C. for 15 h. The solvent was evaporated under reduced pressure. The crude residue was purified by flash silica gel chromatography (0 to 50% EtOAc in petroleum ether) to give 5-((benzyloxy)methyl)-2-oxabicyclo[3.1.0]hexan-4-ol (Int-127H). ¹H NMR (400 MHz, CDCl₃) δ: 7.12-7.33 (m, 5H), 4.41-4.56 (m, 2H), 4.35 (d, J=4.2 Hz, 1H), 3.94 (dd, J=5.6, 2.0 Hz, 1H), 3.83 (d, J=10.7 Hz, 1H), 3.71 (d, J=10.5 Hz, 1H), 3.49-3.65 (m, 2H), 2.94-3.40 (m, 1H), 0.92 (dd, J=7.2, 2.0 Hz, 1H), 0.45 (dd, J=7.2, 5.7 Hz, 1H).

### Step I: 4-azido-5-((benzyloxy)methyl)-2-oxabicyclo[3.1.0]hexane (Int-127I)

To a solution of 5-((benzyloxy)methyl)-2-oxabicyclo[3.1.0]hexan-4-ol (Int-127H) (1.45 g, 6.58 mmol) and PPh₃ (2.59 g, 9.87 mmol) in THF (20 mL) was added diphenylphosphoryl azide (3.62 g, 13.12 mmol), and DIAD (2.0 g, 9.87 mmol) at 0° C. under N₂. The mixture was stirred at 20° C. for 12 h. The reaction was concentrated in vacuo and the crude product was purified by flash silica gel chromatography (0 to 30% EtOAc in petroleum ether) to give 4-azido-5-((benzyloxy)methyl)-2-oxabicyclo[3.1.0]hexane (Int-127I). ¹H NMR (400 MHz, CDCl₃) δ: 7.35-7.48 (m, 5H), 4.60 (s, 2H), 4.49 (t, J=7.8 Hz, 1H), 4.16-4.23 (m, 1H), 3.93 (dd, J=5.6, 2.3 Hz, 1H), 3.68 (d, J=10.5 Hz, 1H), 3.50 (d, J=10.5 Hz, 1H), 3.35 (dd, J=9.8, 7.7 Hz, 1H), 1.44 (dd, J=7.2, 2.2 Hz, 1H), 0.63-0.77 (m, 1H).

### Step J: 5-((benzyloxy)methyl)-N,N-dimethyl-2-oxabicyclo[3.1.0]hexan-4-amine (Int-127J)

To a solution of 4-azido-5-((benzyloxy)methyl)-2-oxabicyclo[3.1.0]hexane (Int-127I) (1.13 g, 4.61 mmol) in MeOH (10 mL) was added paraformaldehyde (300 mg, 4.61 mmol) and Raney Ni (27.0 mg, 0.461 mmol) at 25° C. under N₂. The atmosphere of the reaction was replaced with hydrogen (3×). The reaction was stirred at 25° C. for 15 h under H₂ at 15 psi. The mixture was diluted with MeOH (30 mL), filtered, and the solvent was evaporated under reduced pressure to give racemic 5-((benzyloxy)methyl)-N,N-dimethyl-2-oxabicyclo[3.1.0]hexan-4-amine. The racemic 5-((benzyloxy)methyl)-N,N-dimethyl-2-oxabicyclo[3.1.0]hexan-4-amine (1.12 g, 4.53 mmol) was separated by preparative SFC (Column N, 25% EtOH) to give 5-((benzyloxy)methyl)-N,N-dimethyl-2-oxabicyclo[3.1.0]hexan-4-amine (Int-127J, the first eluting isomer from SFC). MS (ESI) [M+H]⁺: m/z 248.

### Step K: (4-(dimethylamino)-2-oxabicyclo[3.1.0]hexan-5-yl) methanol (Int-127)

To a solution of 5-((benzyloxy)methyl)-N,N-dimethyl-2-oxabicyclo[3.1.0]hexan-4-amine (Int-127J) (200 mg, 0.809 mmol) in MeOH (10 mL) was added palladium on carbon (86 mg, 0.809 mmol) under N₂, The atmosphere of the reaction was replaced with hydrogen (3×) and then stirred at 25° C. for 48 h under hydrogen at 50 psi. The mixture was filtered and washed with MeOH (20 mL). The filtrate was concentrated in vacuum to give crude product. The crude product was purified by preparative TLC (SiO₂, DCM:MeOH=10:1) to give (4-(dimethylamino)-2-oxabicyclo[3.1.0]hexan-5-yl) methanol (Int-127). MS (ESI) [M+H]⁺: m/z 158.

### Intermediate 128-1: Methyl 2-(((benzyloxy) carbonyl)(methyl)amino)-4,4-difluorobicyclo[3.1.0]hexane-1-carboxylate (Int-128-1) & Intermediate 128-2: Methyl 2-(((benzyloxy) carbonyl)(methyl)amino)-4,4-difluorobicyclo[3.1.0]hexane-1-carboxylate (Int-128-2)

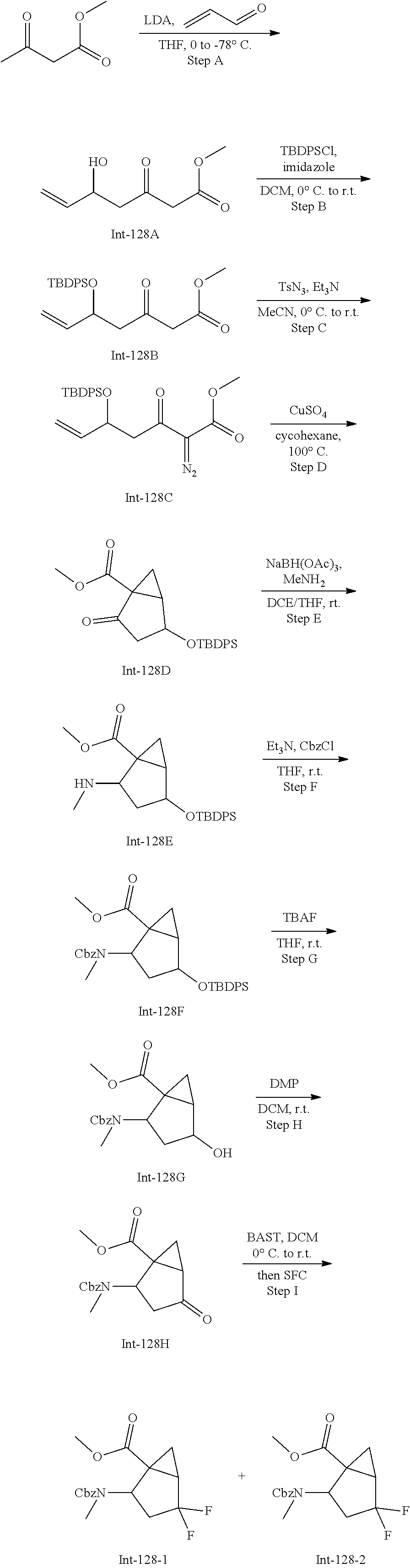

### Step A: Methyl 5-hydroxy-3-oxohept-6-enoate (Int-128A)

A mixture of LDA (51.2 mL, 102 mmol, 2.5M in THF) in THF (150 mL) was cooled to 0° C. under N₂ atmosphere, then methyl 3-oxobutanoate (5.00 g, 43.1 mmol) was added dropwise. After 20 min, the reaction mixture was cooled to −78° C. and acrolein (2.66 g, 47.4 mmol) was added and the reaction was stirred for 20 min at −78° C. The reaction mixture was quenched with sat. aq. NH₄Cl (200 mL) and extracted with EtOAc (3×200 mL). The combined organic layers were dried over Na₂SO₄, filtered, and the solvent was evaporated under reduced pressure to give the crude product. The crude product was purified by flash silica gel chromatography (0 to 30% EtOAc in petroleum ether) to give methyl 5-hydroxy-3-oxohept-6-enoate (Int-128A), ¹H NMR (400 MHz, CDCl₃) δ: 5.85-5.75 (m, 1H), 5.25 (td, J=1.3, 17.3 Hz, 1H), 5.12-5.07 (m, 1H), 4.63-4.50 (m, 1H), 3.70-3.66 (m, 3H), 3.40 (s, 2H), 2.74-2.69 (m, 2H).

### Step B: Methyl 5-((tert-butyldiphenylsilyl)oxy)-3-oxohept-6-enoate (Int-128B)

To a solution of methyl 5-hydroxy-3-oxohept-6-enoate (Int-128A) (7.36 g, 42.7 mmol) in DCM (280 mL) was added 1H-imidazole (8.73 g, 128 mmol) and tert-butylchlorodiphenylsilane (17.6 g, 64.1 mmol) at 0° C., and the mixture was stirred at room temperature for 16 h. The reaction mixture was diluted with DCM (300 mL), filtered, and the filtrate was washed with brine (3×50 mL). The organic phase was dried over Na₂SO₄, filtered, and concentrated in vacuo. The residue was purified by flash silica gel chromatography (0 to 5% EtOAc in petroleum ether) to give methyl 5-((tert-butyldiphenylsilyl)oxy)-3-oxohept-6-enoate (Int-128B). ¹H NMR (400 MHz, CDCl₃) δ: 7.64-7.69 (m, 4H), 7.39-7.43 (m, 6H), 5.82 (J=17.0, 10.5, 6.2 Hz, 1H), 4.91-5.03 (m, 2H), 4.64 (q, J=6.2 Hz, 1H), 3.70 (s, 3H), 3.37 (s, 2H), 2.56-2.77 (m, 2H), 1.06 (s, 9H).

### Step C: Methyl 5-((tert-butyldiphenylsilyl)oxy)-2-diazo-3-oxohept-6-enoate (Int-128C)

To a solution of methyl 5-((tert-butyldiphenylsilyl)oxy)-3-oxohept-6-enoate (Int-128B) (14.5 g, 35.3 mmol) in CH₃CN (145 mL) was added triethylamine (9.84 mL, 70.6 mmol) and 4-methylbenzenesulfonyl azide (10.2 g, 38.8 mmol) at 0° C. under N₂. The mixture was stirred at 0° C. for 2 h and the reaction mixture was stirred at 20° C. for another 2 h. The mixture was diluted with aq. NaOH solution (5 mL, 2M in water) and water (50 mL) and then extracted with EtOAc (2×300 mL). The organic layer was washed with brine (3×50 mL), dried over sodium sulfate, filtered, and the solvent was evaporated under reduced pressure. The residue was purified by preparative TLC (SiO₂, petroleum ether:EtOAc=5:1) to give methyl 5-((tert-butyldiphenylsilyl)oxy)-2-diazo-3-oxohept-6-enoate (Int-128C). ¹H NMR (400 MHz, CDCl₃) δ: 7.62-7.70 (m, 4H), 7.35-7.41 (m, 6H), 5.88 (J=17.1, 10.4, 6.6 Hz, 1H), 4.92-5.12 (m, 2H), 4.75 (q, J=6.6 Hz, 1H), 3.80 (s, 3H), 3.19 (dd, J=15.1, 7.3 Hz, 1H), 3.01 (dd, J=15.1, 5.7 Hz, 1H), 1.05 (s, 9H).

### Step D: Methyl 4-((tert-butyldiphenylsilyl)oxy)-2-oxobicyclo[3.1.0]hexane-1-carboxylate (Int-128D)

To a solution of methyl 5-((tert-butyldiphenylsilyl)oxy)-2-diazo-3-oxohept-6-enoate (Int-128C) (15.7 g, 21.6 mmol) in cyclohexane (160 mL) was added copper (II) sulfate (3.96 g, 24.8 mmol) at room temperature under N₂. The mixture was warmed to 100° C. and stirred for 12 h. The mixture was filtered and the solvent was evaporated under reduced pressure. The residue was purified by flash silica gel chromatography (0 to 30% EtOAc in petroleum ether) to give methyl 4-((tert-butyldiphenylsilyl)oxy)-2-oxobicyclo[3.1.0]hexane-1-carboxylate (Int-128D). ¹H NMR (400 MHz, CDCl₃) δ: 7.50-7.68 (m, 4H), 7.25-7.44 (m, 6H), 4.26-4.64 (m, 1H), 3.55-3.81 (m, 3H), 2.46-2.64 (m, 1H), 2.10-2.41 (m, 2H), 1.91-2.02 (m, 1H), 1.74-1.87 (m, 1H), 0.91-1.03 (m, 9H).

### Step E: Methyl 4-((tert-butyldiphenylsilyl)oxy)-2-(methylamino) bicyclo[3.1.0]hexane-1-carboxylate (Int-128E)

To a solution of methyl 4-((tert-butyldiphenylsilyl)oxy)-2-oxobicyclo[3.1.0]hexane-1-carboxylate (Int-128D) (3.80 g, 9.30 mmol) in DCE (40 mL) was added methylamine (14 mL, 27.9 mmol, 2M in THF) and sodium triacetoxyborohydride (5.91 g, 27.9 mmol) and the mixture was stirred at room temperature for 16 h. The mixture was quenched with water (30 mL) and extracted with EtOAc (3×60 mL). The organic layer was washed with brine (15 mL), dried over sodium sulfate, filtered, and the solvent was evaporated under reduced pressure to give methyl 4-((tert-butyldiphenylsilyl)oxy)-2-(methylamino) bicyclo[3.1.0]hexane-1-carboxylate (Int-128E), which was used directly in the next step without further purification. MS (ESI) [M+H]⁺: m/z 424.

### Step F: Methyl 2-(((benzyloxy) carbonyl)(methyl)amino)-4-((tert-butyldiphenylsilyl)oxy) bicyclo[3.1.0]hexane-1-carboxylate (Int-128F)

To a stirred solution of methyl 4-((tert-butyldiphenylsilyl)oxy)-2-(methylamino) bicyclo[3.1.0]hexane-1-carboxylate (Int-128E) (3.5 g, 3.7 mmol) and triethylamine (1.0 mL, 7.4 mmol) in DCM (40 mL) was added CbzCl (0.64 mL, 4.5 mmol). The mixture was stirred at room temperature for 2.5 h. The mixture was quenched with water (30 mL) and extracted with DCM (2×30 mL). The organic layer was dried over sodium sulfate, filtered, and the solvent was evaporated under reduced pressure. The residue was purified by flash silica gel chromatography (0 to 30% EtOAc in petroleum ether) to give methyl 2-(((benzyloxy) carbonyl)(methyl)amino)-4-((tert-butyldiphenylsilyl)oxy) bicyclo[3.1.0]hexane-1-carboxylate (Int-128F). ¹H NMR (400 MHz, CDCl₃) δ: 7.59-7.75 (m, 4H), 7.30-7.52 (m, 11H), 5.05-5.28 (m, 2H), 4.86-5.02 (m, 1H), 4.35 (s, 1H), 3.22-3.67 (m, 3H), 2.91 (s, 3H), 1.74-2.05 (m, 2H), 1.63 (s, 2H), 1.34-1.49 (m, 1H), 1.06 (s, 9H).

### Step G: Methyl 2-(((benzyloxy) carbonyl)(methyl)amino)-4-hydroxybicyclo[3.1.0]hexane-1-carboxylate (Int-128G)

To a solution of methyl 2-(((benzyloxy) carbonyl)(methyl)amino)-4-((tert-butyldiphenylsilyl)oxy) bicyclo[3.1.0]hexane-1-carboxylate (Int-128F) (1.58 g, 2.83 mmol) in THF (8.5 mL) was added TBAF (8.50 mL, 8.50 mmol, 1M in THF) and the mixture was stirred at room temperature for 16 h. The mixture was evaporated under reduced pressure and the residue was purified by flash silica gel chromatography (0 to 80% EtOAc in petroleum ether) to afford methyl 2-(((benzyloxy) carbonyl)(methyl)amino)-4-hydroxybicyclo[3.1.0]hexane-1-carboxylate (Int-128G). MS (ESI) [M+H]⁺: m/z 320.

### Step H: Methyl 2-(((benzyloxy) carbonyl)(methyl)amino)-4-oxobicyclo[3.1.0]hexane-1-carboxylate (Int-128H)

To a solution of methyl 2-(((benzyloxy) carbonyl)(methyl)amino)-4-hydroxybicyclo[3.1.0]hexane-1-carboxylate (Int-128G) (0.880 g, 2.76 mmol) in DCM (10 mL) was added Dess-Martin periodinane (2.33 g, 5.51 mmol). The reaction mixture was stirred at room temperature for 2 h. The reaction mixture was quenched with sat. aq. Na₂SO₃ (3 mL) and sat. aq. NaHCO₃ solution (3 mL) and the mixture was stirred for 30 min. The reaction mixture was extracted with DCM (3×20 mL). The organic layer was washed with brine (3 mL), dried over sodium sulfate, filtered, and the solvent was evaporated under reduced pressure. The residue was purified by preparative TLC (SiO₂, petroleum ether:EtOAc=1:2) to give methyl 2-(((benzyloxy) carbonyl)(methyl)amino)-4-oxobicyclo[3.1.0]hexane-1-carboxylate (Int-128H). MS (ESI) [M+H]⁺: m/z 318.

### Step I: Methyl 2-(((benzyloxy) carbonyl)(methyl)amino)-4,4-difluorobicyclo[3.1.0]hexane-1-carboxylate (Int-128-1 & Int-128-2)

To a solution of methyl 2-(((benzyloxy) carbonyl)(methyl)amino)-4-oxobicyclo[3.1.0]hexane-1-carboxylate (Int-128H) (652 mg, 2.06 mmol) in DCM (3.2 mL) was added BAST (3.2 mL) at 0° C. The reaction was warmed to room temperature and stirred for 3 h. The mixture was poured into ice/NH₄Cl solution (10 mL). The mixture was extracted with DCM (4×10 mL). The organic layer was dried over sodium sulfate, filtered, and the solvent was evaporated under reduced pressure. The residue was purified by flash silica gel chromatography (0 to 50% EtOAc in petroleum ether) to give racemic methyl 2-(((benzyloxy) carbonyl)(methyl)amino)-4,4-difluorobicyclo[3.1.0]hexane-1-carboxylate. The racemic methyl 2-(((benzyloxy) carbonyl)(methyl)amino)-4,4-difluorobicyclo[3.1.0]hexane-1-carboxylate was separated by preparative SFC (Column K, 5% EtOH w/0.1% NH₄OH) to give methyl 2-(((benzyloxy) carbonyl)(methyl)amino)-4,4-difluorobicyclo[3.1.0]hexane-1-carboxylate (Int-128-1, the first eluting isomer) and methyl 2-(((benzyloxy) carbonyl)(methyl)amino)-4,4-difluorobicyclo[3.1.0]hexane-1-carboxylate (Int-128-2, the second eluting isomer). MS (ESI) [M+H]⁺: m/z 340.

### Intermediate 129; (2-(dimethylamino)-4,4-difluorobicyclo[3.1.0]hexan-1-yl)methanol (Int-129)

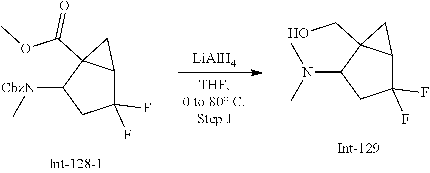 [SMILES: COC(=O)C12CC1C(F)(F)CC2N(C)C(=O)OCc1ccccc1 ; "LiAlH4" ; "THF,0 to 80° C.Step J" ; CN(C)C1CC(F)(F)C2CC12CO]

To a solution of methyl 2-(((benzyloxy) carbonyl)(methyl)amino)-4,4-difluorobicyclo[3.1.0]hexane-1-carboxylate (200 mg, 0.589 mmol) (Int-128-1) in THF (3 mL) was added LiAlH₄ (0.94 mL, 2.36 mmol, 2.5 M in THF) at 0° C. The reaction was warmed to 80° C. and stirred for 1 h. The reaction mixture was cooled to room temperature and quenched with Na₂SO₄ (20 g), EtOAc (20 mL), and H₂O (2 mL) at 0° C. The mixture was warmed to 25° C. and stirred for 0.5 h. The mixture was filtered and the filter cake was washed with EtOAc (30 mL). The organic layer was evaporated under reduced pressure and purified by preparative TLC (DCM:NH₃ (7M in MeOH)=15:1) to give (2-(dimethylamino)-4,4-difluorobicyclo[3.1.0]hexan-1-yl) methanol (Int-129). MS (ESI) [M+H]⁺: m/z 192.

The compound in the table below was synthesized using a similar procedure as described in the synthesis of Int-129 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-130 | 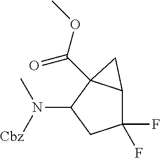 [SMILES: COC(=O)C12CC1C(F)(F)CC2N(C)C(=O)OCc1ccccc1] | 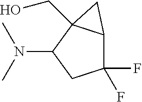 | (2-(dimethylamino)-4,4- difluorobicyclo[3.1.0]hexan-1- yl)methanol | 192 |
| | Int-129-2 | | | |

### Intermediate 131: ((R)-1-((S)-1-(dimethylamino)ethyl)-2,2-difluorocyclopropyl) methanol (Int-131)

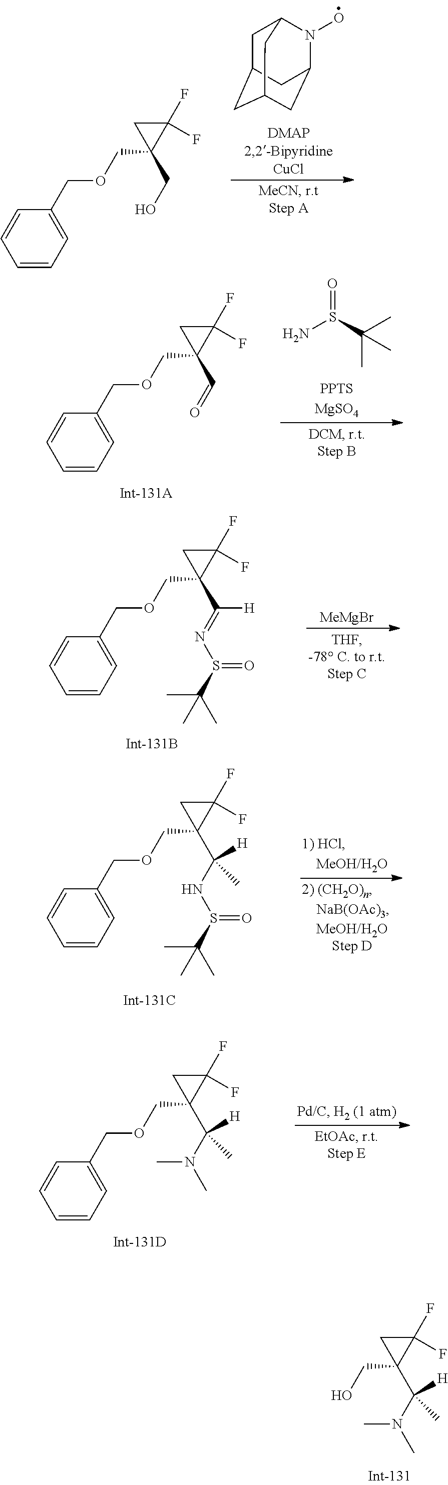

### Step A: (1S)-1-(benzyloxymethyl)-2,2-difluoro-cyclopropanecarbaldehyde (Int-131A)

DMAP (0.32 g, 2.6 mmol), 2,2′-bipyridine (0.21 g, 1.3 mmol), CuCl (0.13 g, 1.3 mmol), and 2-azaadamantane N-oxyl (0.20 g, 1.3 mmol) was added to a solution of [(1R)-1-(benzyloxymethyl)-2,2-difluoro-cyclopropyl]methanol (2.0 g, 8.8 mmol) in MeCN (100 mL) at room temperature. The mixture was stirred at room temperature for 2 h. The reaction was quenched with aq. Na₂S₂O₃, and diluted with EtOAc. The organic layer was separated, washed with HCl (1M in H₂O), H₂O, and brine. The organic layer was dried over Na₂SO₄, filtered, and concentrated in vacuo to afford (1S)-1-(benzyloxymethyl)-2,2-difluoro-cyclopropanecarbaldehyde (Int-131A), which was used directly in the next step without further purification. MS (ESI) [M+H]⁺: m/z 227.

### Step B: (S)—N-((E)-((R)-1-((benzyloxy)methyl)-2,2-difluorocyclopropyl)methylene)-2-methylpropane-2-sulfinamide (Int-131B)

PPTS (89 mg, 0.354 mmol) and magnesium sulfate (2.13 g, 17.7 mmol) was added to a mixture of (1S)-1-(benzyloxymethyl)-2,2-difluoro-cyclopropanecarbaldehyde (Int-131A) (800 mg, 3.54 mmol) and(S)-2-methylpropane-2-sulfinamide (635 mg, 5.30 mmol) in DCM (12 mL). The mixture was stirred at room temperature for 2 days. The mixture was filtered and the filtrate was concentrated under reduced pressure. The residue obtained was purified by silica gel chromatography (hexane/EtOAc=100/0 to 70/30) to afford(S)—N-((E)-((R)-1-((benzyloxy)methyl)-2,2-difluorocyclopropyl)methylene)-2-methylpropane-2-sulfinamide (Int-131B). MS (ESI) [M+H]⁺: m/z 330.

### Step C: (S)—N—((S)-1-((R)-1-((benzyloxy)methyl)-2,2-difluorocyclopropyl)ethyl)-2-methylpropane-2-sulfinamide (Int-131C)

Methylmagnesium bromide (1.30 mL, 3.79 mmol, 3M in Et₂O) was added to a solution of (S)—N—((E)-((R)-1-((benzyloxy)methyl)-2,2-difluorocyclopropyl)methylene)-2-methylpropane-2-sulfinamide (Int-131B) (500 mg, 1.52 mmol) in THF (15 mL) at −78° C. under N₂. The mixture was stirred at −78° C. for 30 min. The mixture was warmed to room temperature and sat. aq. NH₄Cl and EtOAc were added. The organic layer was separated, washed with brine, and dried over Na₂SO₄. The dried solution was filtered and the filtrate was concentrated under reduced pressure. The residue obtained was purified by silica gel chromatography (hexane/EtOAc=100/0 to 80/20) to afford(S)—N—((S)-1-((R)-1-((benzyloxy)methyl)-2,2-difluorocyclopropyl)ethyl)-2-methylpropane-2-sulfinamide (Int-131C). MS (ESI) [M+H]⁺: m/z 346.

### Step D: (S)-1-((R)-1-((benzyloxy)methyl)-2,2-difluorocyclopropyl)-N,N-dimethylethan-1-amine (Int-131D)

HCl (0.408 mL, 1.63 mmol, 4 M in H₂O) was added to a solution of(S)—N—(S)-1-((R)-1-((benzyloxy)methyl)-2,2-difluorocyclopropyl)ethyl)-2-methylpropane-2-sulfinamide (Int-131C) (188 mg, 1.63 mmol) in MeOH (0.564 mL) at room temperature. The mixture was stirred at room temperature for 30 min. The mixture was concentrated under reduced pressure and used directly.

The crude residue was dissolved in MeOH (0.96 mL) and formaldehyde (0.081 mL, 1.39 mmol, 37% in H₂O) and NaBH(OAc) 3 (295 mg, 1.39 mmol) were added at room temperature. The mixture was stirred at room temperature for 30 min. NaOH (1M in H₂O) and EtOAc were added to the mixture. The organic layer was separated, washed with brine, and dried over Na₂SO₄. The dried solution was filtered and the filtrate was concentrated under reduced pressure. The residue obtained was purified by NH-silica gel chromatography (hexane/EtOAc=95/5 to 80/20) to afford(S)-1-((R)-1-((benzyloxy)methyl)-2,2-difluorocyclopropyl)-N,N-dimethylethan-1-amine (Int-131D). MS (ESI) [M+H]⁺: m/z 270.

### Step E: ((R)-1-((S)-1-(dimethylamino)ethyl)-2,2-difluorocyclopropyl)methanol (Int-131)

A mixture of(S)-1-((R)-1-((benzyloxy)methyl)-2,2-difluorocyclopropyl)-N,N-dimethylethan-1-amine (Int-131D) (125 mg, 1.52 mmol) and Pd/C (60 mg, 10 wt %) in EtOAc (2 mL) was stirred at room temperature for 10 h under H₂ atmosphere. The mixture was filtered and the filtrate was concentrated under reduced pressure to afford ((R)-1-((S)-1-(dimethylamino)ethyl)-2,2-difluorocyclopropyl) methanol (Int-131). MS (ESI) [M+H]⁺: m/z 180.

### Intermediate 132: ((25.45)-4-methoxy-1-methylpyrrolidin-2-yl) methanol (Int-132)

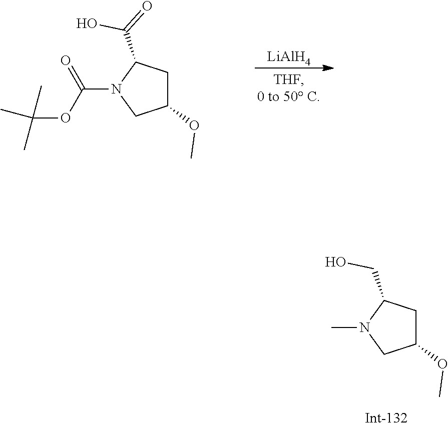

LiAlH₃ (2.0 mL, 10% in THF) was slowly added to a solution of (2S,4S)-1-(tert-butoxycarbonyl)-4-methoxypyrrolidine-2-carboxylic acid (245 mg, 0.999 mmol) in THF (3 mL) at 0° C. and the mixture was warmed to 50° C. and stirred for 1 h. The reaction was cooled to 0° C. and NaOH (0.6 mL, 2 M in H₂O), H₂O (0.9 mL), and Et₂O was added slowly at 0° C. The reaction was warmed to room temperature and stirred 3 h. The resulting solid was filtered off and the filtrate was concentrated to afford ((2S,4S)-4-methoxy-1-methylpyrrolidin-2-yl) methanol (Int-132). MS (ESI) [M+H]⁺: m/z 146.

The compounds in the table below were synthesized using a similar procedure as described in the synthesis of Int-132 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-133 | 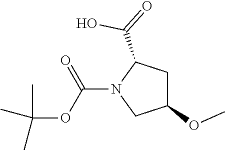 [SMILES: CO[C@@H]1C[C@@H](C(=O)O)N(C(=O)OC(C)(C)C)C1] | 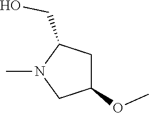 | ((2S,4R)-4-methoxy-1- methylpyrrolidin-2- yl)methanol | 146 |
| Int-134 | 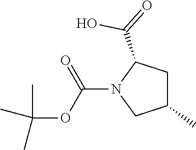 [SMILES: C[C@H]1C[C@@H](C(=O)O)N(C(=O)OC(C)(C)C)C1] | 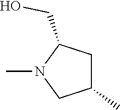 | ((2S,4S)-1,4- dimethylpyrrolidin-2- yl)methanol | 130 |
| Int-135 | 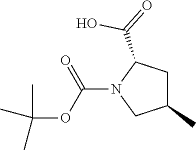 [SMILES: C[C@@H]1C[C@@H](C(=O)O)N(C(=O)OC(C)(C)C)C1] | 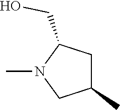 | ((2R,4S)-1,4- dimethylpyrrolidin-2- yl)methanol | 130 |
| Int-136 | 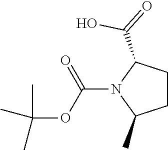 [SMILES: C[C@@H]1CC[C@@H](C(=O)O)N1C(=O)OC(C)(C)C] | 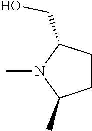 | ((2S,5R)-1,5- dimethylpyrrolidin-2- yl)methanol | 130 |

### Intermediate 137: (S)-4-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Int-137)

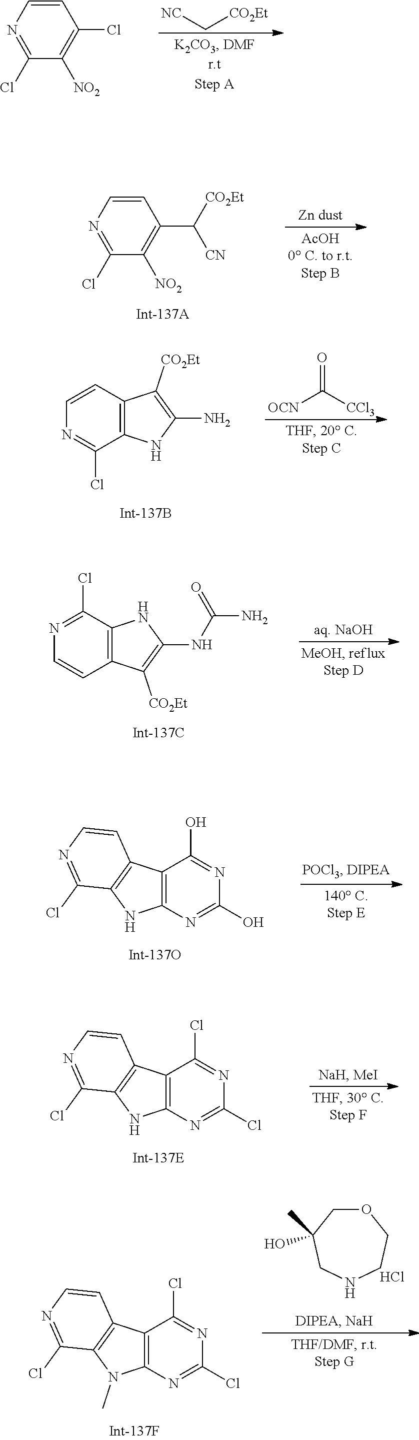

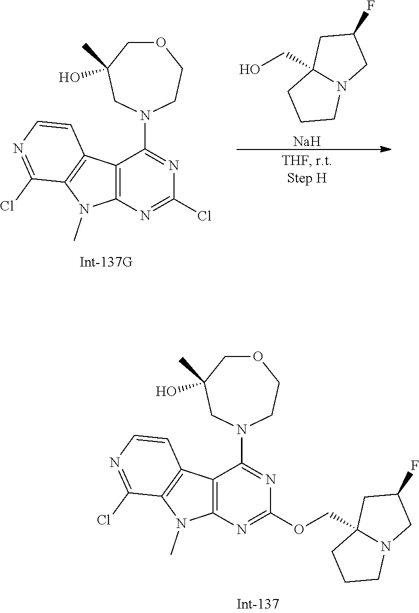

### Step A: Ethyl 2-(2-chloro-3-nitropyridin-4-yl)-2-cyanoacetate (Int-137A)

To a solution of 2,4-dichloro-3-nitropyridine (10.0 g, 51.8 mmol) in DMF (100 mL) was added K₂CO₃ (14.3 g, 104 mmol) and ethyl 2-cyanoacetate (6.15 g, 54.4 mmol) at 25° C. The mixture was stirred at 25° C. for 2 h. After 2 h, LCMS showed the reaction was finished. The reaction mixture was quenched with water (400 mL) and extracted with EtOAc (3×200 mL). The combined organic layers were washed with brine (4×50 mL), dried over Na₂SO₄ and filtered. The filtrate was concentrated under reduced pressure to give a residue which was purified by flash silica gel chromatography (10% EtOAc in petroleum ether) to give ethyl 2-(2-chloro-3-nitropyridin-4-yl)-2-cyanoacetate (Int-137A). MS (ESI) [M+H]⁺: m/z 270. ¹H NMR (400 MHz, DMSO-d₆) δ 8.15 (d, J=6.1 Hz, 1H), 7.95 (s, 1H), 7.80 (d, J=6.1 Hz, 1H), 3.98 (q, J=7.0 Hz, 2H), 1.15 (t, J=6.5 Hz, 3H).

### Step B: Ethyl 2-amino-7-chloro-1H-pyrrolo[2,3-c]pyridine-3-carboxylate (Int-137B)

To a solution of ethyl 2-(2-chloro-3-nitropyridin-4-yl)-2-cyanoacetate (Int-137A) (8 g, 29.7 mmol) in acetic acid (80 mL) was added zinc dust (15.1 g, 231 mmol) at 0° C. The reaction was warmed to 25° C. and stirred for 1 h. The reaction mixture was filtered, and the filtrate was concentrated under reduced pressure to give the residue which was dissolved in THF and filtered again. The filtrate was concentrated under reduced pressure to give a residue which was purified by flash silica gel chromatography (0-15% (7N NH₃ in MeOH) in DCM) to give ethyl 2-amino-7-chloro-1H-pyrrolo[2,3-c]pyridine-3-carboxylate (Int-137B). MS (ESI) [M+H]⁺: m/z 240.

### Step C: ethyl 7-chloro-2-ureido-1H-pyrrolo[2,3-c]pyridine-3-carboxylate (Int-137C)

To a solution of ethyl 2-amino-7-chloro-1H-pyrrolo[2,3-c]pyridine-3-carboxylate (Int-137B) (4.00 g, 16.7 mmol) in THF (70 mL) was added 2,2,2-trichloroacetyl isocyanate (9.43 g, 50.1 mmol) at 20° C. The resulting mixture was stirred at 20° C. for 1 h. LCMS showed the reaction was complete. The solution was concentrated under reduced pressure to give a residue which was diluted with EtOAc (100 mL). The mixture was filtered and the solid was collected and dried in vacuo to give ethyl 7-chloro-2-ureido-1H-pyrrolo[2,3-c]pyridine-3-carboxylate (Int-137C). MS (ESI) [M+H]⁺: m/z 283. ¹H NMR (400 MHz, DMSO-d₆) δ 11.79 (s, 1H), 11.69 (s, 1H), 8.11 (d, J=5.36 Hz, 1H), 7.76 (d, J=5.36 Hz, 1H), 4.39 (q, J=7.03 Hz, 2H), 1.40 (t, J=7.09 Hz, 3H).

### Step D: 8-chloro-9H-pyrido[4′,3′:4.5]pyrrolo[2,3-d]pyrimidine-2,4-diol (Int-137D)

To a solution of ethyl 7-chloro-2-ureido-1H-pyrrolo[2,3-c]pyridine-3-carboxylate (Int-137C) (4.00 g, 14.2 mmol) in MeOH (40 mL) was added sodium hydroxide (50 mL, 75.0 mmol) (1.5 M in water). The reaction mixture was stirred at 90° C. for 16 h. LCMS showed starting material was consumed and desired product was formed. The reaction mixture was concentrated under reduced pressure. The mixture was acidified with HCl (2 M) to pH 3, yellow solid was precipitated and the solid was filtered and collected, dried in vacuo to give 8-chloro-9H-pyrido[4′,3′:4,5]pyrrolo[2,3-d]pyrimidine-2,4-diol (Int-137D) which was used directly in the next step without further purification. MS (ESI) [M+H]⁺: m/z 237. ¹H NMR (400 MHz, DMSO-d₆) δ 12.80 (br s, 1H), 12.10 (br s, 1H), 11.06 (s, 1H), 8.07 (d, J=5.14 Hz, 1H), 7.68 (d, J=5.38 Hz, 1H).

### Step E: 2,4,8-trichloro-9H-pyrido[4′,3′:4,5]pyrrolo[2,3-d]pyrimidine (Int-137E)

A solution of 8-chloro-9H-pyrido[4′,3′:4,5]pyrrolo[2,3-d]pyrimidine-2,4-diol (Int-137D) (1.5 g, 6.34 mmol), N,N-diisopropylethylamine (6.29 mL, 38.0 mmol) in phosphorus oxychloride (18.9 mL, 203 mmol) was stirred at 140° C. in a sealed tube for 48 h. The mixture was concentrated under reduced pressure to give a residue which was purified by flash silica gel chromatography (100% EtOAc) to give 2,4,8-trichloro-9H-pyrido[4′,3′:4,5]pyrrolo[2,3-d]pyrimidine (Int-137E). MS (ESI) [M+H]⁺: m/z 273.

### Step F: 2,4,8-trichloro-9-methyl-9H-pyrido[4′,3′:4,5]pyrrolo[2,3-d]pyrimidine (Int-137F)

To a solution of 2,4,8-trichloro-9H-pyrido[4′,3′:4,5]pyrrolo[2,3-d]pyrimidine (Int-137E) (2.7 g, 9.87 mmol) in THF (50 mL) was added NaH (1.19 g, 29.6 mmol, 60 wt % in mineral oil) followed by iodomethane (1.47 mL, 22.7 mmol). The reaction mixture was stirred at 30° C. for 16 h. LCMS showed the reaction was complete. The reaction was quenched with sat. aq. NH₄Cl (20 mL), extracted with EtOAc (50 mL), and the organic layer was washed with brine (20 mL), and concentrated in vacuo to afford 2,4,8-trichloro-9-methyl-9H-pyrido[4′,3′:4,5]pyrrolo[2,3-d]pyrimidine (Int-137F) which was used directly. MS (ESI) [M+H−Cl+MeO]⁺: m/z 283 (monitored by LCMS using MeOH as solvent).

### Step G: (S)-4-(2,8-dichloro-9-methyl-9H-pyrido[4′,3′:4,5]pyrrolo[2,3-d]pyrimidin-4-yl)-6-methyl-1,4-oxazepam-6-ol (Int-137G)

(S)-6-methyl-1,4-oxazepan-6-ol hydrochloride (1.632 g, 9.74 mmol) was dissolved in DMF (10 mL) and DIPEA (3.40 mL, 19.5 mmol), and the mixture was added to a solution of 2,4,8-trichloro-9-methyl-9H-pyrido[4′,3′:4,5]pyrrolo[2,3-d]pyrimidine (Int-137F) (2.8 g, 9.74 mmol) in THF (40 mL). NaH (0.779 g, 19.5 mmol, 60% in mineral oil) was added at r.t. and the resulting mixture was stirred at r.t. for 16 h. LCMS showed the reaction was complete. The reaction mixture was quenched with sat. aq. NH₄Cl (20 mL), extracted with EtOAc (50 mL), and the organic layer was washed with brine (3×10 mL), dried over Na₂SO₄, filtered, and concentrated in vacuo. The residue was purified by flash silica gel chromatography (30% THF in petroleum ether) to give(S)-4-(2,8-dichloro-9-methyl-9H-pyrido[4′,3′:4,5]pyrrolo[2,3-d]pyrimidin-4-yl)-6-methyl-1,4-oxazepan-6-ol (Int-137G). MS (ESI) [M+H]⁺: m/z 382. ¹H NMR (400 MHz, CDCl₃) δ 8.30 (d, J=5.48 Hz, 1H), 7.58 (d. J=5.36 Hz, 1H), 4.35 (br s, 1H), 4.30 (s, 3H), 4.23 (d, J=15.02 Hz, 1H), 3.98 (td, J=1.91, 12.64 Hz, 1H), 3.72-3.84 (m, 3H), 3.61 (d, J=14.90 Hz, 1H), 3.42-3.53 (m, 1H), 3.34 (d, J=12.52 Hz, 1H), 1.38 (s, 3H).

### Step H: (S)-4-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Int-137)

To a solution of ((2R,7aS)-2-fluorotetrahydro-1H-pyrrolizin-7a(5H)-yl) methanol (1.44 g, 9.03 mmol) in THF (10 mL) was added NaH (0.602 g, 15.04 mmol, 60% in mineral oil). The mixture was added to a stirred solution of(S)-4-(2,8-dichloro-9-methyl-9H-pyrido[4′,3′:4,5]pyrrolo[2,3-d]pyrimidin-4-yl)-6-methyl-1,4-oxazepan-6-ol (Int-137G) (2.3 g, 6.02 mmol) in THF (30 mL) at r.t. The reaction was stirred at r.t. for 1 h. LCMS showed starting material was consumed and desired production was formed. The mixture was quenched with sat. aq. NH₄Cl (20 mL), extracted with EtOAc (50 mL), washed with brine (20 mL), dried over Na₂SO₄, and concentrated in vacuum. The residue was purified by flash silica gel chromatography (80% THF in petroleum ether) to give(S)-4-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Int-137). MS (ESI) [M+H]⁺: m/z 505. ¹H NMR (400 MHz, CDCl₃) δ 8.20 (d, J=5.48 Hz, 1H), 7.48 (d, J=5.36 Hz, 1H), 6.86 (br s, 1H), 5.23-5.37 (m, 1H), 4.28-4.36 (m, 2H), 4.22 (s, 3H), 4.17 (S, 1H), 4.14 (br d, J=4.41 Hz, 1H), 3.94 (br dd, J=2.44, 12.58 Hz, 1H), 3.75-3.80 (m, 1H), 3.67-3.74 (m, 1H), 3.53 (d, J=14.90 Hz, 1H), 3.37 (dt, J=3.34, 11.98 Hz, 1H), 3.31 (br d, J=12.40 Hz, 2H), 3.25 (s, 1H), 3.17 (brd, J=5.72 Hz, 1H), 2.99 (dt, J=4.83, 9.03 Hz, 1H), 2.22-2.32 (m, 1H), 2.11-2.22 (m, 2H), 1.88-2.01 (m, 3H), 1.37 (s, 3H).

### Intermediate 138: (R)-1-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3-methylpiperidin-3-ol (Int-138)

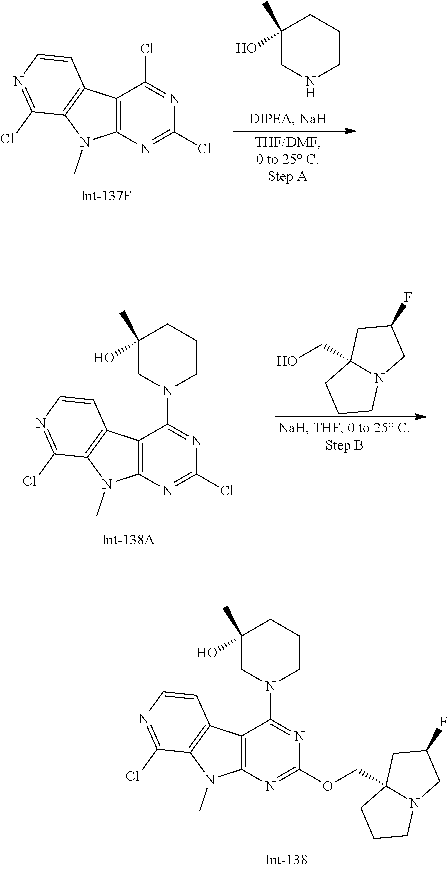

### Step A: (R)-1-(2,8-dichloro-9-methyl-9H-pyrido[4′,3′:4,5]pyrrolo[2,3-d]pyrimidin-4-yl)-3-methylpiperidin-3-ol (Int-138A)

(R)-3-methylpiperidin-3-ol (182 mg, 1.39 mmol) was dissolved in a solution of DMF (2 mL) and DIPEA (0.486 mL, 2.78 mmol), and then the mixture was added to a solution of 2,4,8-trichloro-9-methyl-9H-pyrido[4′,3′:4,5]pyrrolo[2,3-d]pyrimidine (Int-138F) (400 mg, 1.391 mmol) in THF (5 mL). NaH (111 mg, 2.78 mmol, 60 wt % in mineral oil) was added at 0° C. The resulting mixture was stirred at 25° C. for 16 h. The reaction was quenched with sat. aq. NH₄Cl (5 mL) and extracted with EtOAc (30 mL). The organic layer was washed with brine (5 mL), dried over Na₂SO₄, filtered, and concentrated in vacuo. The residue was purified by flash silica gel chromatography (0˜30% THF in petroleum ether) to give (R)-1-(2,8-dichloro-9-methyl-9H-pyrido[4′,3′:4,5]pyrrolo[2,3-d]pyrimidin-4-yl)-3-methylpiperidin-3-ol (Int-138A). MS (ESI) [M+H]⁺: m/z 366.

### Step B: (R)-1-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3-methylpiperidin-3-ol (Int-138)

To a solution of ((2R,7aS)-2-fluorotetrahydro-1H-pyrrolizin-7a(5H)-yl) methanol (189 mg, 1.19 mmol) in THF (2 mL) was added NaH (79 mg, 1.980 mmol, 60% in mineral oil) at 0° C. The mixture was added to a stirred solution of (R)-1-(2,8-dichloro-9-methyl-9H-pyrido[4′,3′:4,5]pyrrolo[2,3-d]pyrimidin-4-yl)-3-methylpiperidin-3-ol (Int-138A) (290 mg, 0.792 mmol) in THF (3.0 mL) at 25° C. The reaction was stirred at 25° C. for 1 h. The mixture was quenched with sat. aq. NH₄Cl (5 mL), extracted with EtOAc (20 mL) and washed with brine (5 mL), dried over Na₂SO₄, filtered, and concentrated in vacuo. The residue was purified by flash silica gel chromatography (0-60% THF in petroleum ether) to give (R)-1-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3-methylpiperidin-3-ol (Int-138). MS (ESI) [M+H]⁺; m/z 489.

The compound in the table below was synthesized using a similar procedure as described in the synthesis of Int-138 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int-139 | 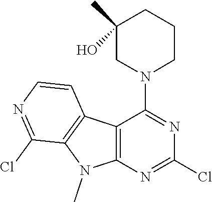 [SMILES: Cn1c2nc(Cl)nc(N3CCC[C@@](C)(O)C3)c2c2ccnc(Cl)c21] | 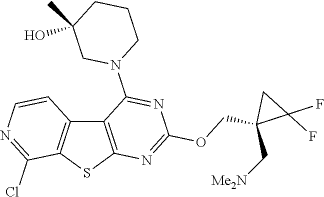 [SMILES: CN(C)C[C@@]1(COc2nc(N3CCC[C@@](C)(O)C3)c3c(n2)sc2c(Cl)nccc23)CC1(F)F] | (R)-1-(8-chloro-2-(((R)- 1-((dimethylamino) methyl)-2,2-difluoro- cyclopropyl)methoxy)- 9-methyl-9H-pyrido [4′,3′:4,5]pyrrolo[2,3- d]pyrimidin-4-yl)-3- methylpiperidin-3-ol | 495 |
| | Int-138A | | | |

### Intermediate 140: 2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-ol (Int-140)

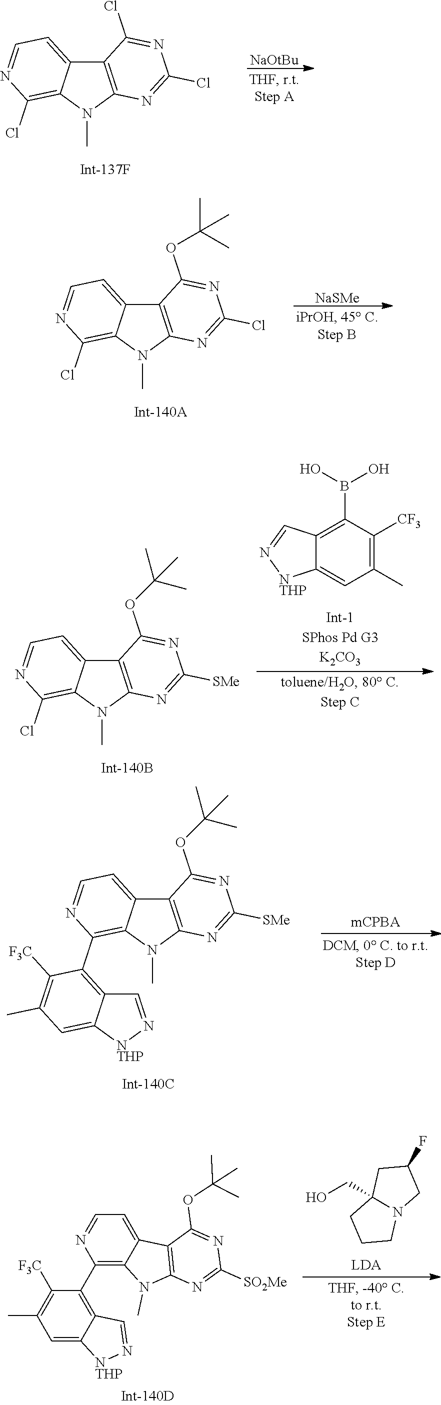

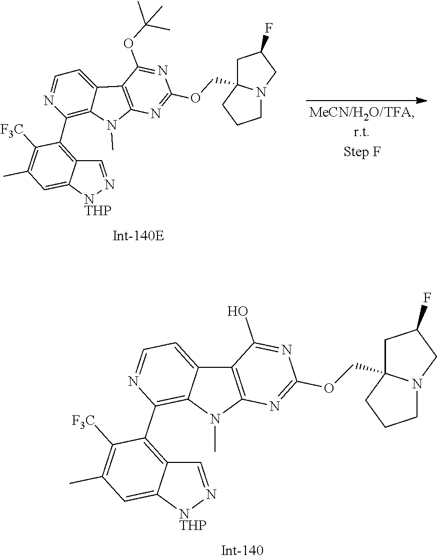

### Step A: 4-(tert-butoxy)-2,8-dichloro-9-methyl-9H-pyrido[4′,3′:4,5]pyrrolo[2,3-d]pyrimidine (Int-140A)

A 50 mL round bottom flask with stir bar was charged with 2,4,8-trichloro-9-methyl-9H-pyrido[4′,3′:4,5]pyrrolo[2,3-d]pyrimidine (Int-137F) (1.05 g, 3.65 mmol). The flask was fitted with a septum and placed under nitrogen. THF (18.3 ml) was added via syringe. Then sodium tert-butoxide (1.92 ml, 3.83 mmol) was added dropwise. The reaction was stirred at r.t. for 2 h. After 2 h, sat. aq. NaHCO₃ (20 mL) was added and the reaction was diluted with EtOAc (30 mL). The layers were transferred to a separatory funnel, shaken, and separated. The aqueous phase was washed with EtOAc (2×30 mL). The combined organics were washed with brine (50 mL), dried over sodium sulfate, filtered, and concentrated. The crude material was purified via silica gel chromatography (0 to 4% (3:1 EtOAc:EtOH) in DCM) to obtain 4-(tert-butoxy)-2,8-dichloro-9-methyl-9H-pyrido[4′,3′:4,5]pyrrolo[2,3-d]pyrimidine (Int-140A), [M+H]⁺: m/z 325.

### Step B: 4-(tert-butoxy)-8-chloro-9-methyl-2-(methylthio)-9H-pyrido[4′,3′:4,5]pyrrolo[2,3-d]pyrimidine (Int-140B)

A vial with stir bar was charged with 4-(tert-butoxy)-2,8-dichloro-9-methyl-9H-pyrido[4′,3′:4,5]pyrrolo[2,3-d]pyrimidine (Int-140A) (434 mg, 1.34 mmol) and sodium thiomethoxide (140 mg, 2.00 mmol) and placed under nitrogen. 2-Propanol (8.9 mL) was added via syringe and the reaction was heated to 45° C. for 2 hours. After 2 hours, LCMS indicated the reaction was complete. The reaction was diluted with sat. aq. NH₄Cl (5 mL) and EtOAc (10 mL). The contents were transferred to a separatory funnel, shaken, and separated. The aqueous phase was washed with EtOAc (2×10 mL). The combined organics were washed with brine (20 mL), dried over sodium sulfate, filtered, and concentrated. 4-(tert-butoxy)-8-chloro-9-methyl-2-(methylthio)-9H-pyrido[4′,3′:4,5]pyrrolo[2,3-d]pyrimidine was used directly in the next step without further purification (Int-140B). [M+H]⁺: m/z 337.

### Step C: 4-(tert-butoxy)-9-methyl-8-(6-methyl-1-(tetrahydro-2H-pyran-2-yl)-5-(trifluoromethyl)-1H-indazol-4-yl)-2-(methylthio)-9H-pyrido[4′,3′:4,5]pyrrolo[2,3-d]pyrimidine (Int-140C)

A mixture of 4-(tert-butoxy)-8-chloro-9-methyl-2-(methylthio)-9H-pyrido[4′,3′:4,5]pyrrolo[2,3-d]pyrimidine (Int-140B) (280 mg, 0.831 mmol), (6-methyl-1-(tetrahydro-2H-pyran-2-yl)-5-(trifluoromethyl)-1H-indazol-4-yl)boronic acid (Int-1) (545 mg, 1.66 mmol), SPhos Pd G3 (324 mg, 0.416 mmol), and K₂CO₃ (1.25 mL, 2.49 mmol, 2M in H₂O) was added to a round bottom flask with stir bar and dissolved in toluene (11.9 mL). The flask was fitted with a septum and the solution was sparged with nitrogen for 15 min. The reaction was heated at 80° C. for 3 hrs. The reaction mixture was diluted with EtOAc (20 mL), washed with brine (50 mL), dried over sodium sulfate, filtered, and concentrated. The crude mixture was purified by silica gel chromatography (0-100% EtOAc in hexane) to provide 4-(tert-butoxy)-9-methyl-8-(6-methyl-1-(tetrahydro-2H-pyran-2-yl)-5-(trifluoromethyl)-1H-indazol-4-yl)-2-(methylthio)-9H-pyrido[4′,3′:4,5]pyrrolo[2,3-d]pyrimidine (Int-140C). MS (ESI) [M+H]⁺: m/z 585.

### Step D: 4-(tert-butoxy)-9-methyl-8-(6-methyl-1-(tetrahydro-2H-pyran-2-yl)-5-(trifluoromethyl)-1H-indazol-4-yl)-2-(methylsulfonyl)-9H-pyrido[4′,3′:4,5]pyrrolo[2,3-d]pyrimidine (Int-140D)

A vial with a stir bar was charged with 4-(tert-butoxy)-9-methyl-8-(6-methyl-1-(tetrahydro-2H-pyran-2-yl)-5-(trifluoromethyl)-1H-indazol-4-yl)-2-(methylthio)-9H-pyrido[4′,3′:4,5]pyrrolo[2,3-d]pyrimidine (Int-140C) (292 mg, 0.5 mmol). DCM (3.3 mL) was added, and the vial was cooled to 0° C. in an ice/water bath. mCPBA (215 mg, 1.249 mmol) was added in a single portion. The reaction was warmed to r.t. and allowed to stir for 1.5 h. After 1.5 h, the reaction was diluted with DCM (5 mL) and washed with sat. ag. NaHCO₃ (5 mL) and water (3×5 mL). The organic phase was dried over sodium sulfate, filtered, and concentrated. 4-(tert-butoxy)-9-methyl-8-(6-methyl-1-(tetrahydro-2H-pyran-2-yl)-5-(trifluoromethyl)-1H-indazol-4-yl)-2-(methylsulfonyl)-9H-pyrido[4′,3′:4,5]pyrrolo[2,3-d]pyrimidine (Int-140D) was used directly in the next step without further purification. MS (ESI) [M+H]⁺: m/z 617.

### Step E: 4-(tert-butoxy)-2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidine (Int-140E)

A vial with a stir bar was charged with ((2R,7aS)-2-fluorotetrahydro-1H-pyrrolizin-7a(5H)-yl) methanol (159 mg, 1.00 mmol), fitted with a septum, and placed under nitrogen. THF (3.9 ml) was added via syringe and the reaction was cooled to 0° C. Lithium diisopropylamide (1.10 mL, 1.10 mmol, 1 M in THF) was added dropwise to make a 0.2 M solution. A separate vial with stir bar was charged with 4-(tert-butoxy)-9-methyl-8-(6-methyl-1-(tetrahydro-2H-pyran-2-yl)-5-(trifluoromethyl)-1H-indazol-4-yl)-2-(methylsulfonyl)-9H-pyrido[4′,3′:4,5]pyrrolo[2,3-d]pyrimidine (Int-140D) (308 mg, 0.5 mmol), fitted with a septum, and placed under nitrogen. THF (2.5 mL) was added via syringe and the solution was cooled to −40° C. with a dry ice/MeCN bath. The lithium alkoxide solution (3.75 mL, 0.75 mmol, 0.2 M in THF) was added dropwise via syringe to the sulfone solution. The reaction was warmed to r.t. and allowed to stir for 1 h. The reaction was quenched with sat. aq. NH₄Cl (2 mL) and diluted with EtOAc (2 mL). The layers were shaken and separated, and the aqueous phase was washed with EtOAc (2×2 mL). The combined organics were dried over sodium sulfate, filtered, and concentrated. The crude material was purified via silica gel chromatography (0% to 50% (3:1 EtOAc:EtOH) in hexanes) to obtain 4-(tert-butoxy)-2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidine (Int-140E). MS (ESI) [M+H]⁺: m/z 696.

### Step F: 2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-ol (Int-140)

4-(tert-butoxy)-2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidine (Int-140E) (69.0 mg, 0.099 mmol) was added to a vial with a stir bar and dissolved in MeCN (763 μL), water (191 μL), and TFA (38.1 μL). The reaction was allowed to stir at room temperature for 45 minutes. LCMS indicated full conversion to the desired product. The reaction was quenched with sat. aq. NaHCO₃ (2 mL) and diluted with EtOAc (2 mL). The layers were shaken, separated, and the aqueous phase was washed with EtOAc (2×2 mL). The combined organics were washed with brine (5 mL), dried over sodium sulfate, filtered, and concentrated to 2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-ol (Int-140). MS (ESI) [M+H]⁺: m/z 640.

### Intermediate 141: (6S)-4-(6-chloro-2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol (Int-141)

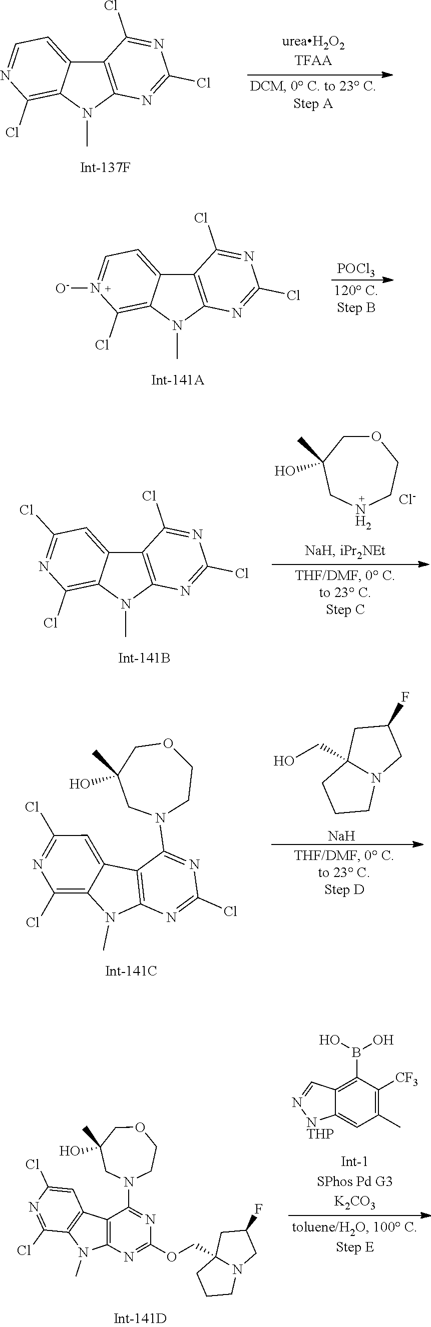

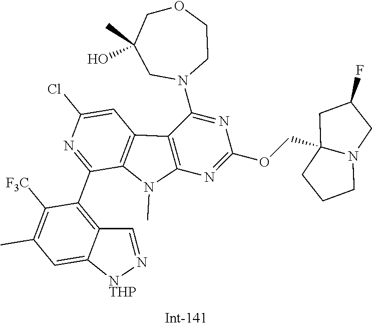

### Step A: 2,4,8-trichloro-9-methyl-9H-pyrido[4′,3′:4,5]pyrrolo[2,3-d]pyrimidine 7-oxide (Int-141A)

A vial with a stir bar was charged with 2,4,8-trichloro-9-methyl-9H-pyrido[4′,3′:4,5]pyrrolo[2,3-d]pyrimidine (Int-141F) (574 mg, 1.996 mmol) and carbamide peroxide (urea·H₂O₂) (225 mg, 2.34 mmol). The vial was fitted with a septum and DCM (20 mL) was added via syringe. The solution was cooled to 0° C. and trifluoroacetic anhydride (0.31 mL, 2.2 mmol) was added via syringe. The reaction was warmed to r.t. for 17 h. Additional trifluoroacetic anhydride (0.17 mL, 1.12 mmol) and carbamide peroxide (113 mg, 1,198 mmol) were added and the reaction was stirred for an additional 3 h. After 3 h, sat. aq. sodium thiosulfate (20 mL) was added followed by DCM (20 mL). The layers were shaken and separated. The aqueous phase was washed with DCM (20 mL) and the combined organics were washed with brine (20 mL). The combined organics were dried over sodium sulfate, filtered, and concentrated to give 2,4,8-trichloro-9-methyl-9H-pyrido[4′,3′:4,5]pyrrolo[2,3-d]pyrimidine 7-oxide (Int-141A). MS (ESI): m/z (M+H)⁺ 303.

### Step B: 2,4,6,8-tetrachloro-9-methyl-9H-pyrido[4′,3′:4,5]pyrrolo[2,3-d]pyrimidine (Int-141B)

A microwave vial was charged with 2,4,8-trichloro-9-methyl-9H-pyrido[4′,3′:4,5]pyrrolo[2,3-d]pyrimidine 7-oxide (Int-141A) (606 mg, 2.00 mmol) and phosphorus oxychloride (18.6 ml, 200 mmol). The vial was sealed and heated in a microwave reactor at 120° C. for 60 min. After 60 min, the vial was cooled to r.t. and added dropwise into cold sat. aq. sodium bicarbonate (500 mL). The solution was diluted with DCM (500 mL) and transferred to a separatory funnel. The layers were shaken and separated. The aqueous phase was washed with DCM (2×100 mL). The combined organics were washed with brine (200 mL), dried over sodium sulfate, filtered, and concentrated. The crude residue was purified via silica gel chromatography (0% to 20% EtOAc in hexanes) to give 2,4,6,8-tetrachloro-9-methyl-9H-pyrido[4′,3′:4,5]pyrrolo[2,3-d]pyrimidine (Int-141B). ¹H NMR (500 MHz, CDCl₃) δ 8.19 (s, 1H), 4.31 (s, 3H).

### Step C: (S)-6-methyl-4-(2,6,8-trichloro-9-methyl-9H-pyrido[4′,3′:4,5]pyrrolo[2,3-d]pyrimidin-4-yl)-1,4-oxazepan-6-ol (Int-141C)

2,4,6,8-tetrachloro-9-methyl-9H-pyrido[4′,3′:4,5]pyrrolo[2,3-d]pyrimidine (Int-141B) (447 mg, 1.40 mmol) and(S)-6-methyl-1,4-oxazepan-6-ol hydrochloride (233 mg, 1.388 mmol) were added to a 250 mL round bottom flask with a stir bar. The flask was fitted with a septum and placed under nitrogen. DMF (1.4 mL) and THF (4.2 mL) were added via syringe and the reaction was cooled to 0° C. N,N-diisopropylethylamine (0.49 mL, 2.78 mmol) was added dropwise followed by sodium hydride (111 mg, 2.78 mmol, 60 wt % in mineral oil) and the reaction was allowed to warm to r.t. over 2 h. After 2 h, the reaction was quenched with water (50 mL) and DCM (50 mL) and transferred to a separatory funnel. The layers were shaken and separated, and the aqueous phase was washed with DCM (2×50 mL). The combined organics were dried over sodium sulfate, filtered, and concentrated. Purified via silica gel chromatography (0% to 30% EtOAc in hexanes) to give(S)-6-methyl-4-(2,6,8-trichloro-9-methyl-9H-pyrido[4′,3′:4,5]pyrrolo[2,3-d]pyrimidin-4-yl)-1,4-oxazepan-6-ol (Int-141C). MS (ESI): m/z (M+H)⁺ 416.

### Step D: (S)-4-(6,8-dichloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Int-141D)

A 100 mL round bottom flask with a stir bar was charged with (S)-6-methyl-4-(2,6,8-trichloro-9-methyl-9H-pyrido[4′,3′:4,5]pyrrolo[2,3-d]pyrimidin-4-yl)-1,4-oxazepan-6-ol (Int-141C) (384 mg, 0.922 mmol) and ((2R,7aS)-2-fluorotetrahydro-1H-pyrrolizin-7a(5H)-yl) methanol (161 mg, 1.014 mmol). The vial was fitted with a septum and placed under nitrogen. THF (9.2 ml) was added and the reaction was cooled to 0° C. Sodium hydride (92 mg, 2.304 mmol, 60 wt % in mineral oil) was added in one portion and the reaction was allowed to warm to r.t. After 1 h, DMF (9.2 ml) was added via syringe and the reaction was allowed to stir for 1 h. The reaction was quenched with sat. aq. ammonium chloride (25 mL). EtOAc (50 mL) was added and the contents were transferred to a separatory funnel. The layers were shaken and separated. The aqueous phase was washed with EtOAc (2×25 mL). The combined organics were washed with brine (100 mL), dried over sodium sulfate, filtered, and concentrated. The crude residue was purified via silica gel chromatography (0% to 50% (3:1 EtOAc:EtOH) in hexanes) to give(S)-4-(6,8-dichloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Int-141D). MS (ESI): m/z (M+H)⁺ 539.

### Step E: (6S)-4-(6-chloro-2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol (Int-141)

A vial with a stir bar was charged with (S)-4-(6,8-dichloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Int-141D) (280 mg, 0.519 mmol), (6-methyl-1-(tetrahydro-2H-pyran-2-yl)-5-(trifluoromethyl)-1H-indazol-4-yl)boronic acid (Int-1) (213 mg, 0.649 mmol) and SPhos Pd G3 (405 mg, 0.519 mmol).

The vial was fitted with a septum and potassium carbonate (2 M in H₂O, 0.52 mL, 1.038 mmol) and toluene (4.7 mL) were added via a syringe. The reaction was sparged with N₂ and heated to 100° C. for 1 hr. The reaction mixture was cooled to r.t., diluted with EtOAc (10 mL), and washed with brine solution (10 mL). The organic phase was dried over sodium sulfate, filtered, and concentrated. The crude residue was purified by silica gel chromatography (0 to 50% (3:1 EtOAc:EtOH) in hexane) to give (6S)-4-(6-chloro-2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol (Int-141). MS (ESI): m/z (M+H)⁺ 787.

The compound in the table below was synthesized using a similar procedure as described in the synthesis of Int-141 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int- 142 | 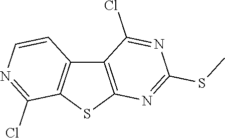 [SMILES: CSc1nc(Cl)c2c(n1)sc1c(Cl)nccc12] Int-1431 | 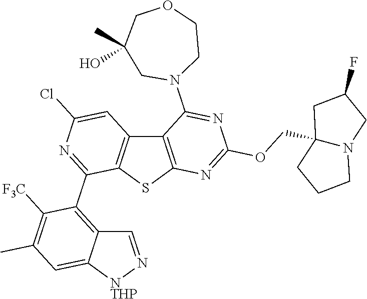 | (6S)-4-(6-chloro- 2-(((2R,7aS)-2- fluorotetrahydro-1H- pyrrolizin-7a(5H)- yl)methoxy)-8- (6-methyl-1- (tetrahydro-2H- pyran-2-yl)-5- (trifluoromethyl)-1H- indazol-4- yl)pyrido[4′,3′:4,5] thieno[2,3- d]pyrimidin-4- yl)-6-methyl- 1,4-oxazepan-6-ol | 790 |

### Intermediate 143: (S)-4-(8-chloro-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Int-143)

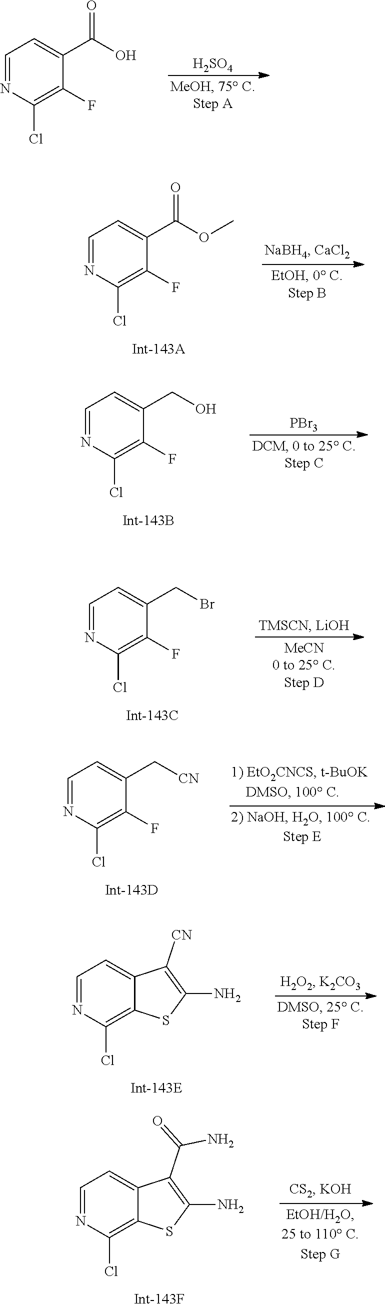

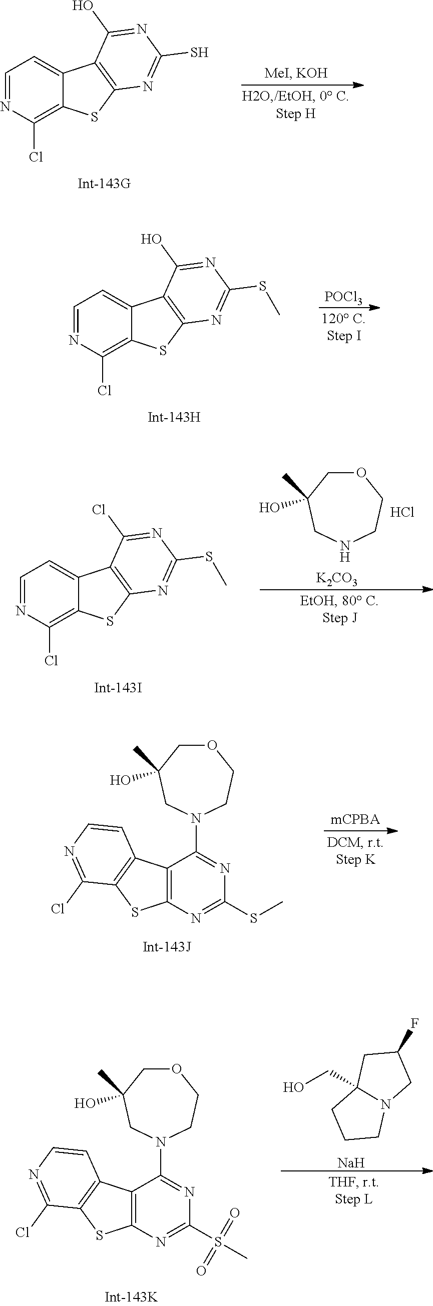

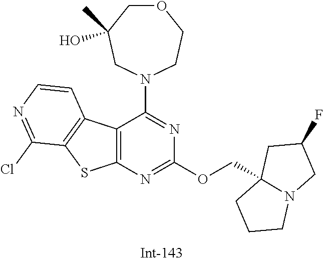 [SMILES: C[C@@]1(O)COCCN(c2nc(OC[C@@]34CCCN3C[C@H](F)C4)nc3sc4c(Cl)nccc4c23)C1]

### Step A: methyl 2-chloro-3-fluoroisonicotinate (Int-143A)

Eight reactors were set up in parallel. To each reactor was charged MeOH (4.0 L) at 25° C., 2-chloro-3-fluoroisonicotinic acid (400 g, 2.3 mol), and conc. H₂SO₄ (45.0 g, 0.4 mol). The mixture was heated to 75° C. for 12 h. The contents of the eight reactors were combined and concentrated to remove MeOH. The pH of the resulting residue was adjusted to 7 using aq. Na₂CO₃ and extracted with EtOAc (20 L×2). The combined organics were dried with Na₂SO₄ and concentrated under reduced pressure to give methyl 2-chloro-3-fluoroisonicotinate (Int-143A) which was used directly in the next step without further purification.

### Step B: (2-chloro-3-fluoropyridin-4-yl) methanol (Int-143B)

Twelve reactors were set up in parallel. To each reactor was charged EtOH (2.3 L), methyl 2-chloro-3-fluoroisonicotinate (Int-143A) (235 g, 1.2 mol), and CaCl₂) (206 g, 1.8 mol). The reactor was degassed and purged with N₂ times and cooled to 0˜10° C. NaBH₄ (93.0 g, 2.4 mol) was added at 0˜10° C. and stirred for 1 h. The mixture was warmed to 20˜25° C. for 12 hrs. The contents of the twelve reactors were combined for workup. The reaction was slowly poured into ice water (5.0 V) under N₂ atmosphere, filtered, and the filter cake was washed with EtOAc (5.0 V). The filtrate was concentrated under reduced pressure to give (2-chloro-3-fluoropyridin-4-yl) methanol (Int-143B). ¹H NMR (400 MHz, CDCl₃) δ 2.91 (s, 1H), 4.85 (s, 2H), 7.45-7.46 (t, J=4.0 Hz, 1H), 8.17-8.18 (d, J=4.0 Hz, 1H).

### Step C: 4-(bromomethyl)-2-chloro-3-fluoropyridine (Int-143C)

Twelve reactors were set up in parallel. To each reactor was charged DCM (2.0 L) and (2-chloro-3-fluoropyridin-4-yl) methanol (Int-143B) (200 g, 1.2 mol). PBr₃ (402 g, 1.5 mol) was charged into the reactor vessel at 0° C. The vessel was warmed to 25° C. and stirred for 12 h. The contents of the twelve reactors were combined for workup and poured into 10% aq. NaHCO₃ (1.0 L) slowly. The reaction mixture was concentrated under reduced pressure. The resulting residue was diluted with DCM (20 L) and the organic phase was separate. The aqueous solution was extracted with DCM (20 L×2). The combined organic layers were dried with Na₂SO₄, filtered, and concentrated under reduce pressure to give 4-(bromomethyl)-2-chloro-3-fluoropyridine (Int-143C). MS (ESI) [M+H]⁺: m/z 224.

### Step D: 2-(2-chloro-3-fluoropyridin-4-yl) acetonitrile (Int-143D)

Eight reactors were set up in parallel. To each reactor was charged MeCN (2.3 L), 4-(bromomethyl)-2-chloro-3-fluoropyridine (Int-143C) (230 g, 1.0 mol), TMSCN (1.01 kg, 10 mol), and LiOH (51.0 g, 1.2 mol). The mixture was stirred at 0° C. for 4 h and 25° C. for 8 h. The contents of the eight reactors were combined and concentrated to remove MeCN. The reaction was diluted with EtOAc (25 L) and H₂O (10 L) and the organic phase was separated. The aqueous phase was extracted with EtOAc (10 L×2). The combined organics were dried with Na₂SO₄, filtered, and concentrated to give 2-(2-chloro-3-fluoropyridin-4-yl) acetonitrile (Int-143D). MS (ESI) [M+H]⁺: m/z 171.

### Step E: 2-amino-7-chlorothieno[2,3-c]pyridine-3-carbonitrile (Int-143E)

Twelve reactors were set up in parallel. To each reactor was charged DMSO (1.7 L) and 2-(2-chloro-3-fluoropyridin-4-yl) acetonitrile (Int-143D) (170 g, 1.00 mol). The reactor was degassed and purged with N₂ three times. Potassium tert-butoxide (123 g, 1.1 mol) was added in batches at 25° C. over 30 min. The mixture was stirred at 25° C. for 30 min. After 30 min, ethoxycarbonyl isothiocyanate (143 g, 1.1 mol) was added at 25° C. The mixture was stirred at 25° C. for 30 min. The reactor was warmed to 100° C. and stirred at 100° C. for 1 h. NaOH (1 μL, 5.0 equiv., 5 M in H₂O) was added and the mixture was stirred at 100° C. for 10 h. The reaction was cooled and the contents of the 12 reactors were combined for workup. The reaction was poured into ice water (10 V) and stirred at 0° C. for 30 min. The resulting solid was filtered, washed with H₂O (5.0 L), and dried in an oven at 50° C. for 12 hr to obtain 2-amino-7-chlorothieno[2,3-c]pyridine-3-carbonitrile (Int-143E). MS (ESI) [M+H]⁺: m/z 210.

### Step F: 2-amino-7-chlorothieno[2,3-c]pyridine-3-carboxamide (Int-143F)

Six reactors were set up in parallel. To each reactor was charged DMSO (2.1 L), 2-amino-7-chlorothieno[2,3-c]pyridine-3-carbonitrile (Int-143E) (70.0 g, 0.3 mol), and K₂CO₃ (92.0 g, 0.6 mol). H₂O₂ (615 g, 5.4 mol, 30% wt %) was added slowly into the reactor in portions over 4 h. The reaction mixture was stirred at 25° C. for 6 h. The contents of the six reactors were combined for workup. The reaction was poured into 10% aq. Na₂SO₃ at 15-25° C. and the mixture was stirred at 0° C. for 0.5 h. The reaction mixture was filtered and the filter cake was washed with H₂O (5.0 L). The resulting solid was dried in an oven at 50° C. for 12 h to give 2-amino-7-chlorothieno[2,3-c]pyridine-3-carboxamide (Int-143F). MS (ESI) [M+H]⁺: m/z 228. ¹H NMR (400 MHz, DMSO-d₆) δ 7.19-7.20 (d. J=4.0 Hz, 1H), 8.10-8.11 (d, J=4.0 Hz, 1H).

### Step G: 8-chloro-2-mercaptopyrido[4′,3′:4,5]thieno[2,3-d]pyrimidin-4-ol (Int-143G)

Five reactors were set up in parallel. To each reactor was charged EtOH (0.5 L) and KOH (52.0 g, 0.9 mol). The mixture was stirred at 25° C. for 0.5 hr. CS₂ (70.0 g, 0.9 mol) was added at 25° C. and the mixture was stirred at 25° C. for 0.5 hr. H₂O (0.5 L) was added followed by 2-amino-7-chlorothieno[2,3-c]pyridine-3-carboxamide (Int-143F) (70.0 g, 0.3 mol). The mixture was heated to 110° C. for 12 hrs. The contents of the five reactors were combined for workup. The reaction was concentrated to remove EtOH and the pH was adjusted to 3. The mixture was stirred at 0° C. for 1 hr, filtered, and washed with H₂O (5.0 L). The resulting solid was triturated with EtOH (10 V) at 25° C. for 8 hr and dried in an oven at 40° C. for 12 hr to give 8-chloro-2-mercaptopyrido[4′,3′:4,5]thieno[2,3-d]pyrimidin-4-ol (Int-143G). MS (ESI) [M+H]⁺: m/z 270.

### Step H: 8-chloro-2-(methylthio)pyrido[4′,3′:4,5]thieno[2,3-d]pyrimidin-4-ol (Int-143H)

Two reactors were set up in parallel. To each reactor was charged EtOH (1.0 L), H₂O (1.0 L), 8-chloro-2-mercaptopyrido[4′,3′:4,5]thieno[2,3-d]pyrimidin-4-ol (Int-143G) (135 g, 0.5 mol), and KOH (56.0 g, 1.0 mol). The mixture was stirred at 0° C. for 0.5 h. MeI (85.0 g, 0.6 mol) was added at 0° C. and stirred for 1.5 h. The contents of the two reactors were combined for workup. The reaction mixture was concentrated to remove EtOH and the pH was adjusted to 3. The mixture was stirred at 0° C. for 1 h, filtered, and washed with H₂O (5.0 L). The resulting solid was triturated with EtOH (10 V) at 25° C. for 8 h and dried in an oven at 40° C. for 12 h to give 8-chloro-2-(methylthio)pyrido[4′,3′:4,5]thieno[2,3-d]pyrimidin-4-ol (Int-143H), which may exist in a tautomeric form. MS (ESI) [M+H]⁺: m/z 284. ¹H NMR (400 MHz, DMSO-d₆) δ 2.62 (s, 3H), 8.16-8.17 (d, J=4.0 Hz, 1H), 8.44-8.45 (d, J=4.0 Hz, 1H), 13.4 (s, 1H).

### Step I: 4,8-dichloro-2-(methylthio)pyrido[4′,3′:4,5]thieno[2,3-d]pyrimidine (Int-143D

Three reactors were set up in parallel. To each reactor was charged POCl₃ (2.02 kg, 13 mol) and 8-chloro-2-(methylthio)pyrido[4′,3′:4,5]thieno[2,3-d]pyrimidin-4-ol (Int-143H) (55.0 g, 0.20 mol). The reaction mixture was stirred at 120° C. for 5 h. The contents of the three reactors were combined for workup. The reaction was concentrated to remove POCl₃ and triturated with EtOH (10 V) at 25° C. for 8 h. The resulting solid was dried in an oven at 40° C. for 12 h to give 4,8-dichloro-2-(methylthio)pyrido[4′,3′:4,5]thieno[2,3-d]pyrimidine (Int-143I). MS (ESI) [M+H]⁺: m/z 302. ¹H NMR (400 MHz, DMSO-d₆) δ 2.67 (s, 3H), 8.48-8.49 (d, J=4.0 Hz, 1H), 8.65-8.66 (d, J=4.0 Hz, 1H).

### Step J: (S)-4-(8-chloro-2-(methylthio)pyrido[4′,3′:4,5]thieno[2,3-d]pyrimidin-4-yl)-6-methyl-1,4-oxazepam-6-ol (Int-143)

A mixture of 4,8-dichloro-2-(methylthio)pyrido[4′,3′:4,5]thieno[2,3-d]pyrimidine (Int-143I) (2 g, 6.6 mmol), (S)-6-methyl-1,4-oxazepan-6-ol hydrochloride (1.1 g, 6.6 mmol), and K₂CO₃ (2.7 g, 20 mmol) in EtOH (30 mL) were heated at 80° C. for 2 h. The reaction mixture was cooled to r.t. and water was added. The solid obtained was filtered and dried under vacuum overnight to obtain(S)-4-(8-chloro-2-(methylthio)pyrido[4′,3′:4,5]thieno[2,3-d]pyrimidin-4-yl)-6-methyl-1,4-oxazepan-6-ol (Int-143J). MS (ESI): m/z (M+H)⁺ 397.

### Step K: (S)-4-(8-chloro-2-(methylsulfonyl)pyrido[4′,3′:4,5]thieno[2,3-d]pyrimidin-4-yl)-6-methyl-1,4-oxazepan-6-ol (Int-143K)

To a solution of(S)-4-(8-chloro-2-(methylthio)pyrido[4′,3′:4,5]thieno[2,3-d]pyrimidin-4-yl)-6-methyl-1,4-oxazepan-6-ol (Int-143J) (1.10 g, 2.77 mmol) in DCM (22 mL) was added mCPBA (1.43 g, 8.31 mmol). The reaction was stirred at r.t. for 3 h, diluted with DCM, washed with sat. aq. NaHCO₃ solution, brine, and concentrated to obtain(S)-4-(8-chloro-2-(methylsulfonyl)pyrido[4′,3′:4,5]thieno[2,3-d]pyrimidin-4-yl)-6-methyl-1,4-oxazepan-6-ol (Int-143K). MS (ESI): m/z (M+H)⁺ 429.

### Step L: (S)-4-(8-chloro-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Int-143)

To a mixture of ((2R,7aS)-2-fluorotetrahydro-1H-pyrrolizin-7a(5H)-yl) methanol (0.640 g, 4.02 mmol) and sodium hydride (0.214 g, 5.36 mmol, 60 wt % in mineral oil) was added THF (7 mL) under nitrogen. The reaction was stirred for 1 h. To this reaction was added (S)-4-(8-chloro-2-(methylsulfonyl)pyrido[4′,3′:4,5]thieno[2,3-d]pyrimidin-4-yl)-6-methyl-1,4-oxazepan-6-ol (Int-143K) (1.15 g, 2.68 mmol) in THF (21 mL). The reaction was stirred at r.t. overnight. The reaction mixture was quenched with sat. aq. NH₄Cl and extracted with ethyl acetate. The combined organic layers were washed with brine solution and concentrated. The product was purified by silica gel chromatography (0 to 100% EtOAc in hexane) to obtain(S)-4-(8-chloro-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Int-143). MS (ESI): m/z (M+H)⁺ 508.

The compounds in the table below were synthesized using a similar procedure as described in the synthesis of Int-143 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | | [M + H]⁺ |
|---|---|---|---|---|
| Int. | Starting Material | Structure | Compound Name | Found |
| Int- 144 | 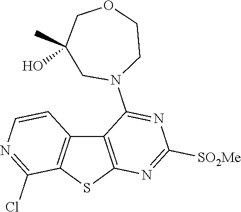 [SMILES: C[C@@]1(O)COCCN(c2nc(S(C)(=O)=O)nc3sc4c(Cl)nccc4c23)C1] | 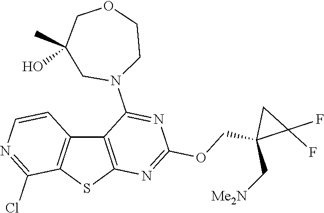 [SMILES: CN(C)C[C@@]1(COc2nc(N3CCOC[C@@](C)(O)C3)c3c(n2)sc2c(Cl)nccc23)CC1(F)F] | (S)-4-(8-chloro- 2-(((R)-1- ((dimethylamino) methyl)-2,2-difluoro- cyclopropyl)methoxy) pyrido[4′,3′:4,5]thieno [2,3-d]pyrimidin-4- yl)-6-methyl-1,4- oxazepan-6-ol | 514 |
| | Int-142K | | | |
| Int- 145 | 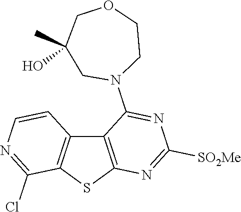 [SMILES: C[C@@]1(O)COCCN(c2nc(S(C)(=O)=O)nc3sc4c(Cl)nccc4c23)C1] | 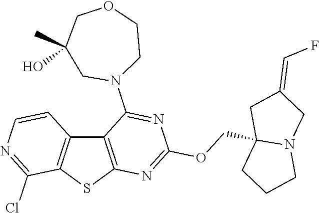 [SMILES: C[C@@]1(O)COCCN(c2nc(OC[C@@]34CCCN3C/C(=C\F)C4)nc3sc4c(Cl)nccc4c23)C1] | (S)-4-(8-chloro- 2-(((S,Z)-2-(fluoro- methylene)tetrahydro- 1H-pyrrolizin-7a(5H)- yl)methoxy) pyrido[4′,3′:4,5] thieno[2,3-d] pyrimidin-4- yl)-6-methyl-1,4- oxazepan-6-ol | 520 |
| | Int-143K | | | |
| | 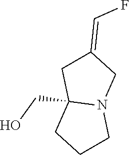 [SMILES: OC[C@@]12CCCN1C/C(=C\F)C2] | | | |
| | Int-119 | | | |
| Int- 146 | 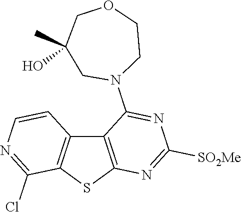 [SMILES: C[C@@]1(O)COCCN(c2nc(S(C)(=O)=O)nc3sc4c(Cl)nccc4c23)C1] | 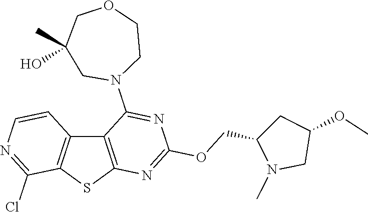 [SMILES: CO[C@H]1C[C@@H](COc2nc(N3CCOC[C@@](C)(O)C3)c3c(n2)sc2c(Cl)nccc23)N(C)C1] | (S)-4-(8-chloro-2- (((2S,4S)-4-methoxy-1- methylpyrrolidin-2- yl)methoxy) pyrido[4′,3′:4,5] thieno[2,3-d] pyrimidin-4- yl)-6-methyl-1,4- oxazepan-6-ol | 494 |
| | Int-143K | | | |
| | 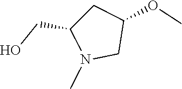 | | | |
| | Int-132 | | | |
| Int- 147 | 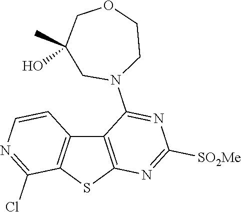 [SMILES: C[C@@]1(O)COCCN(c2nc(S(C)(=O)=O)nc3sc4c(Cl)nccc4c23)C1] | 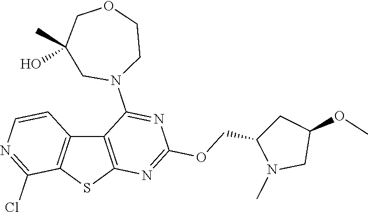 [SMILES: CO[C@@H]1C[C@@H](COc2nc(N3CCOC[C@@](C)(O)C3)c3c(n2)sc2c(Cl)nccc23)N(C)C1] | (S)-4-(8-chloro-2- (((2S,4R)-4-methoxy-1- methylpyrrolidin-2- yl)methoxy) pyrido[4′,3′:4,5] thieno[2,3-d] pyrimidin-4- yl)-6-methyl-1,4- oxazepan-6-ol | 494 |
| | Int-143K | | | |
| | 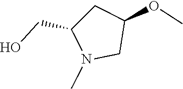 | | | |
| | Int-133 | | | |
| Int- 148 | 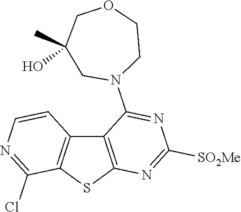 [SMILES: C[C@@]1(O)COCCN(c2nc(S(C)(=O)=O)nc3sc4c(Cl)nccc4c23)C1] | 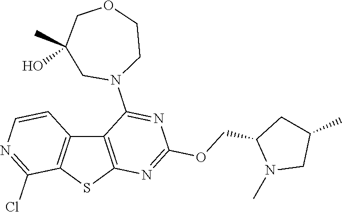 [SMILES: C[C@H]1C[C@@H](COc2nc(N3CCOC[C@@](C)(O)C3)c3c(n2)sc2c(Cl)nccc23)N(C)C1] | (S)-4-(8-chloro-2- (((2S,4S)-1,4- dimethylpyrrolidin-2- yl)methoxy) pyrido[4′,3′:4,5] thieno[2,3-d] pyrimidin-4- yl)-6-methyl-1,4- oxazepan-6-ol | 478 |
| | Int-143K | | | |
| | 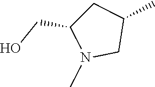 | | | |
| | Int-134 | | | |
| Int- 149 | 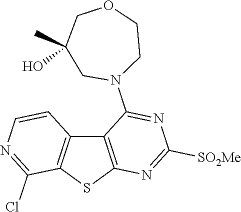 [SMILES: C[C@@]1(O)COCCN(c2nc(S(C)(=O)=O)nc3sc4c(Cl)nccc4c23)C1] | 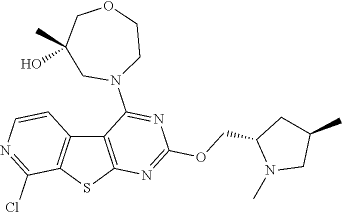 [SMILES: C[C@@H]1C[C@@H](COc2nc(N3CCOC[C@@](C)(O)C3)c3c(n2)sc2c(Cl)nccc23)N(C)C1] | (S)-4-(8-chloro-2- (((2S,4R)-1,4- dimethylpyrrolidin-2- yl)methoxy) pyrido[4′,3′:4,5] thieno[2,3-d] pyrimidin-4- yl)-6-methyl-1,4- oxazepan-6-ol | 478 |
| | Int-143K | | | |
| | 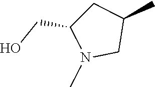 | | | |
| | Int-135 | | | |
| Int- 150 | 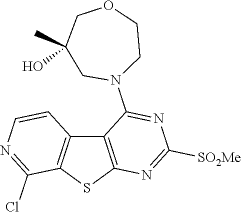 [SMILES: C[C@@]1(O)COCCN(c2nc(S(C)(=O)=O)nc3sc4c(Cl)nccc4c23)C1] | 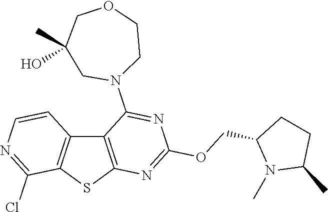 [SMILES: C[C@@H]1CC[C@@H](COc2nc(N3CCOC[C@@](C)(O)C3)c3c(n2)sc2c(Cl)nccc23)N1C] | (S)-4-(8-chloro-2- (((2S,5R)-1,5- dimethylpyrrolidin-2- yl)methoxy) pyrido[4′,3′:4,5] thieno[2,3-d] pyrimidin-4- yl)-6-methyl-1,4- oxazepan-6-ol | 478 |
| | Int-143K | | | |
| | 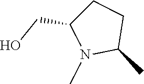 | | | |
| | Int-136 | | | |
| Int- 151 | 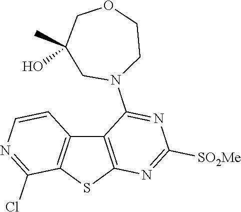 [SMILES: C[C@@]1(O)COCCN(c2nc(S(C)(=O)=O)nc3sc4c(Cl)nccc4c23)C1] | 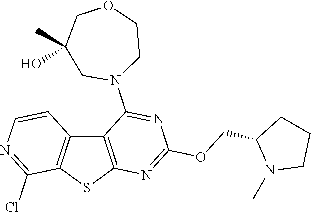 [SMILES: CN1CCC[C@H]1COc1nc(N2CCOC[C@@](C)(O)C2)c2c(n1)sc1c(Cl)nccc12] | (S)-4-(8-chloro- 2-(((S)-1- methylpyrrolidin-2- yl)methoxy) pyrido[4′,3′:4,5] thieno[2,3-d] pyrimidin-4- yl)-6-methyl-1,4- oxazepan-6-ol | 464 |
| | Int-143K | | | |
| Int- 152 | 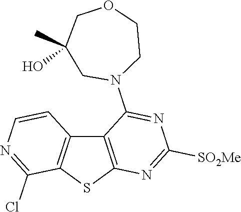 [SMILES: C[C@@]1(O)COCCN(c2nc(S(C)(=O)=O)nc3sc4c(Cl)nccc4c23)C1] | 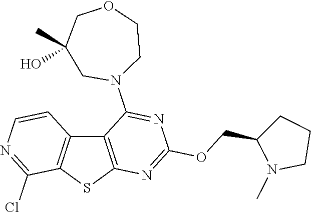 [SMILES: CN1CCC[C@@H]1COc1nc(N2CCOC[C@@](C)(O)C2)c2c(n1)sc1c(Cl)nccc12] | (S)-4-(8-chloro- 2-(((R)-1- methylpyrrolidin-2- yl)methoxy) pyrido[4′,3′:4,5] thieno[2,3-d] pyrimidin-4- yl)-6-methyl-1,4- oxazepan-6-ol | 464 |
| | Int-143K | | | |
| Int- 153 | 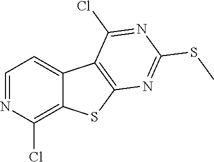 [SMILES: CSc1nc(Cl)c2c(n1)sc1c(Cl)nccc12] Int-143I | 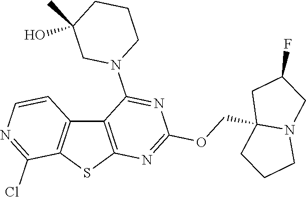 [SMILES: C[C@@]1(O)CCCN(c2nc(OC[C@@]34CCCN3C[C@H](F)C4)nc3sc4c(Cl)nccc4c23)C1] | (R)-1-(8-chloro-2- (((2R,7aS)-2- fluorotetrahydro-1H- pyrrolizin-7a(5H)- yl)methoxy) pyrido[4′,3′:4,5] thieno[2,3-d] pyrimidin-4- yl)-3-methyl- piperidin-3-ol | 492 |

### Intermediate 154: 4,8-dibromo-2-(methylthio)pyrido[4′,3′:4,5]thieno[2,3-d]pyrimidine (Int-154)

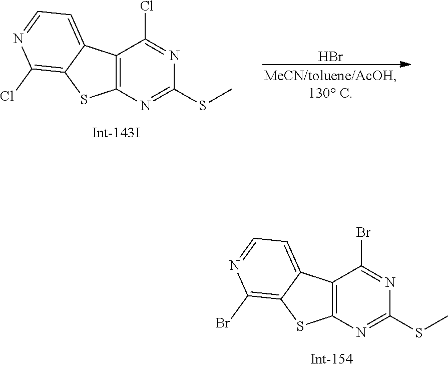 [SMILES: CSc1nc(Cl)c2c(n1)sc1c(Cl)nccc12 ; Br ; CSc1nc(Br)c2c(n1)sc1c(Br)nccc12]

4,8-dichloro-2-(methylthio)pyrido[4′,3′:4,5]thieno[2,3-d]pyrimidine (Int-143I) (2.00 g, 6.62 mmol) was added into a 250 mL round bottom flask with stir bar. MeCN (25.5 mL), toluene (25.5 mL), and hydrogen bromide (10.96 mL, 66.2 mmol, 33 wt % in AcOH) were added and a reflux condenser was attached to the flask. The reaction was stirred at 130° C. for 17 h. The reaction was cooled to room temperature and filtered to provide 4,8-dibromo-2-(methylthio)pyrido[4′,3′:4,5]thieno[2,3-d]pyrimidine (Int-154). MS (ESI) [M+H]⁺: m/z 390, 392.

The compounds in the table below were synthesized using a similar procedure as described in the synthesis of Int-143 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int- 155 | 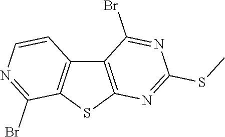 [SMILES: CSc1nc(Br)c2c(n1)sc1c(Br)nccc12] Int-154 | 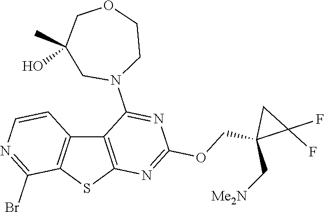 [SMILES: CN(C)C[C@@]1(COc2nc(N3CCOC[C@@](C)(O)C3)c3c(n2)sc2c(Br)nccc23)CC1(F)F] | (S)-4-(8-bromo-2-(((R)-1- ((dimethylamino)methyl)- 2,2-difluoro- cyclopropyl)methoxy) pyrido[4′,3′:4,5]thieno [2,3-d]pyrimidin-4-yl)-6- methyl-1,4-oxazepan-6-ol | 558, 560 |
| Int- 156 | 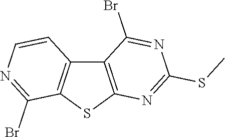 [SMILES: CSc1nc(Br)c2c(n1)sc1c(Br)nccc12] Int-154 | 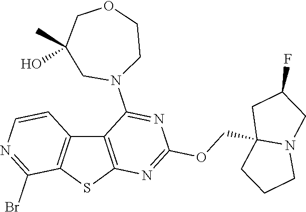 [SMILES: C[C@@]1(O)COCCN(c2nc(OC[C@@]34CCCN3C[C@H](F)C4)nc3sc4c(Br)nccc4c23)C1] | (S)-4-(8-bromo-2- (((2R,7aS)-2- fluorotetrahydro-1H- pyrrolizin-7a(5H)- yl)methoxy)pyrido[4′,3′:4,5] thieno[2,3-d]pyrimidin-4- yl)-6-methyl-1,4- oxazepan-6-ol | 552, 554 |

### Intermediate 157:4-(benzyloxy)-8-chloro-2-(((2R,7aS)-2-fluorotetrahydro-1H-pyrrolizin-7a(5H)-yl)methoxy)pyrido[4′,3′:4,5]thieno[2,3-d]pyrimidine (Int-157)

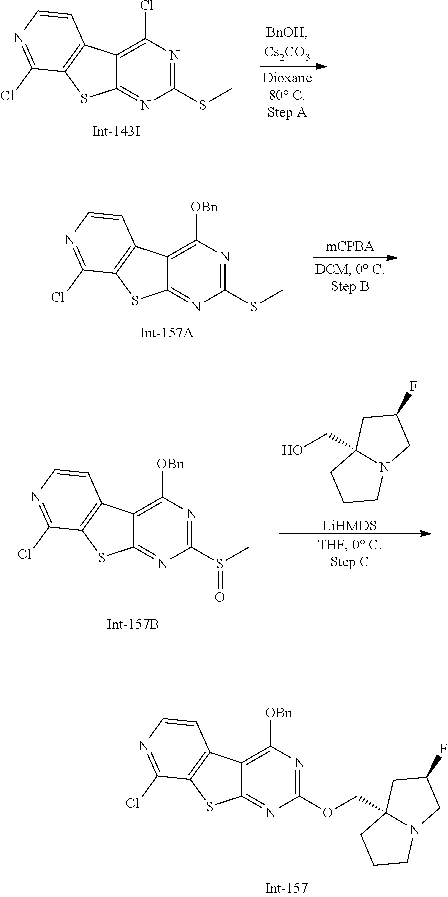

### Step A: 4-(benzyloxy)-8-chloro-2-(methylthio)pyrido[4′,3′:4,5]thieno[2,3-d]pyrimidine (Int-157A)

Benzyl alcohol (1.0 mL, 9.9 mmol) and Cs₂CO₃ (3.3 g, 10 mmol) were added to a solution of 4,8-dichloro-2-(methylthio)pyrido[4′,3′:4,5]thieno[2,3-d]pyrimidine (Int-143I) (1.0 g, 3.3 mmol) in 1,4-dioxane (17 mL). The mixture was heated to 80° C. and stirred for 5 h. After 5 h, the reaction was cooled to room temperature. Water was added and the resulting solid was collected by filtration. The solid was heated to 50° C. under vacuum to dry, thereby obtaining 4-(benzyloxy)-8-chloro-2-(methylthio)pyrido[4′,3′:4,5]thieno[2,3-d]pyrimidine (Int-157A). MS (ESI): m/z (M+H)⁺ 374.

### Step B: 4-(benzyloxy)-8-chloro-2-(methylsulfinyl)pyrido[4′,3′:4,5]thieno[2,3-d]pyrimidine (Int-157B)

mCPBA (610 mg, 2.6 mmol) was added to a solution of 4-(benzyloxy)-8-chloro-2-(methylthio)pyrido[4′,3′:4,5]thieno[2,3-d]pyrimidine (Int-157A) (900 mg, 2.5 mmol) in DCM (60 mL) at 0° C. The mixture was stirred at 0° C. for 30 min. EtOAc, water, and sat. aq. NaHCO₃ were added and the layers were separated. The organic phase was washed with sat. aq. NaHCO₃ and brine, dried over sodium sulfate, filtered, and concentrated to give crude 4-(benzyloxy)-8-chloro-2-(methylsulfinyl)pyrido[4′,3′:4,5]thieno[2,3-d]pyrimidine (Int-157B), which was used directly in the next step without further purification. MS (ESI): m/z (M+H)⁺ 390.

### Step C: 4-(benzyloxy)-8-chloro-2-(((2R,7aS)-2-fluorotetrahydro-1H-pyrrolizin-7a(5H)-yl)methoxy)pyrido[4′,3′:4,5]thieno[2,3-d]pyrimidine (Int-157)

LiHMDS (4.8 mL, 4.8 mmol, 1 M in THF) was added to a solution of ((2R,7aS)-2-fluorotetrahydro-1H-pyrrolizin-7a(5H)-yl) methanol (770 mg, 4.8 mmol) in THF (24 mL) at 0° C. The mixture was stirred at 0° C. for 20 min. The solution of crude 4-(benzyloxy)-8-chloro-2-(methylsulfinyl)pyrido[4′,3′:4,5]thieno[2,3-d]pyrimidine (Int-157B) in THF (24 mL) was added dropwise at 0° C. and the reaction was stirred at 0° C. for 15 min. Water, EtOAc, and sat. aq. NaHCO₃ were added at 0° C. and the organic phase was washed with sat. aq. NaHCO₃ and brine, dried over sodium sulfate, filtered, and concentrated. The crude mixture was purified via silica gel chromatography (0 to 3% MeOH in chloroform) to give 4-(benzyloxy)-8-chloro-2-(((2R,7aS)-2-fluorotetrahydro-1H-pyrrolizin-7a(5H)-yl)methoxy)pyrido[4′,3′:4,5]thieno[2,3-d]pyrimidine (Int-157). MS (ESI): m/z (M+H)⁺ 485.

### Intermediate 158: (6S)-4-(8-(6-chloro-5-cyclopropyl-1-(tetrahydro-2H-pyran-2-yl)-1H-indazol-4-yl)-2-(methylsulfonyl)pyrido[4′,3′:4,5]thieno[2,3-d]pyrimidin-4-yl)-6-methyl-1,4-oxazepan-6-ol (Int-158)

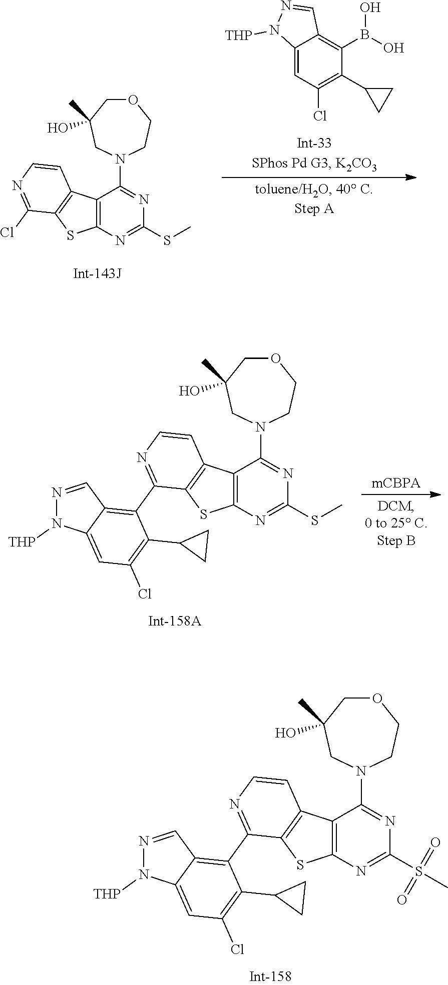

### Step A: (6S)-4-(8-(6-chloro-5-cyclopropyl-1-(tetrahydro-2H-pyran-2-yl)-1H-indazol-4-yl)-2-(methylthio)pyrido[4′,3′:4,5]thieno[2,3-d]pyrimidin-4-yl)-6-methyl-1,4-oxazepan-6-ol (Int-158A)

To a solution of(S)-4-(8-chloro-2-(methylthio)pyrido[4′,3′:4,5]thieno[2,3-d]pyrimidin-4-yl)-6-methyl-1,4-oxazepan-6-ol (Int-143J) (2.00 g, 5.04 mmol) in toluene (20 mL) was added (6-chloro-5-cyclopropyl-1-(tetrahydro-2H-pyran-2-yl)-1H-indazol-4-yl)boronic acid (Int-33) (2.18 g, 6.80 mmol), K₂CO₃ (12.6 mL, 25.2 mmol, 2 M in H₂O) and (2-dicyclohexylphosphino-2′,6′-dimethoxybiphenyl) [2-(2′-amino-1,1′-biphenyl)]palladium (II) methanesulfonate (1.97 g, 2.52 mmol) at 25° C. The reaction mixture was heated to 40° C. and stirred for 1 h under N₂. The reaction mixture was cooled to room temperature, diluted with H₂O (10 mL), and extracted with DCM (20 mL×3). The combined organic layers were dried over Na₂SO₄, filtered, and concentrated in vacuo. The crude residue was purified by flash silica gel chromatography (0 to 50% EtOAc in petroleum ether) to give (6S)-4-(8-(6-chloro-5-cyclopropyl-1-(tetrahydro-2H-pyran-2-yl)-1H-indazol-4-yl)-2-(methylthio)pyrido[4′,3′:4,5]thieno[2,3-d]pyrimidin-4-yl)-6-methyl-1,4-oxazepan-6-ol (Int-158A). MS (ESI) [M+H]⁺: m/z 637.

### Step B: (6S)-4-(8-(6-chloro-5-cyclopropyl-1-(tetrahydro-2H-pyran-2-yl)-1H-indazol-4-yl)-2-(methylsulfonyl)pyrido[4′,3′:4,5]thieno[2,3-d]pyrimidin-4-yl)-6-methyl-1,4-oxazepan-6-ol (Int-158)

To a solution of (6S)-4-(8-(6-chloro-5-cyclopropyl-1-(tetrahydro-2H-pyran-2-yl)-1H-indazol-4-yl)-2-(methylthio)pyrido[4′,3′:4,5]thieno[2,3-d]pyrimidin-4-yl)-6-methyl-1,4-oxazepan-6-ol (Int-158A) (1.10 g, 1.73 mmol) in DCM (20 mL) was added mCPBA (0.946 g, 4.66 mmol) at 0° C. The reaction was warmed to 25° C. and stirred for 1.5 h. The reaction mixture was quenched with sat. aq. NaHCO₃ (5 mL) and Na₂SO₃ (10 mL), extracted with DCM (3×20 mL). The combined organic layers were dried over Na₂SO₄ and concentrated in vacuo. The crude was purified by flash silica gel chromatography (0 to 25% (3:1 EtOAc/EtOH) in petroleum ether gradient) to give (6S)-4-(8-(6-chloro-5-cyclopropyl-1-(tetrahydro-2H-pyran-2-yl)-1H-indazol-4-yl)-2-(methylsulfonyl)pyrido[4′,3′:4,5]thieno[2,3-d]pyrimidin-4-yl)-6-methyl-1,4-oxazepan-6-ol (Int-158). MS (ESI) [M+H]⁺: m/z 669.

The compounds in the table below were synthesized using a similar procedure as described in the synthesis of Int-158 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int- 159 | 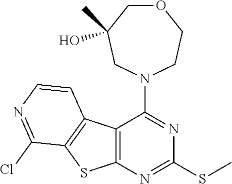 [SMILES: CSc1nc(N2CCOC[C@@](C)(O)C2)c2c(n1)sc1c(Cl)nccc12] Int-143J | 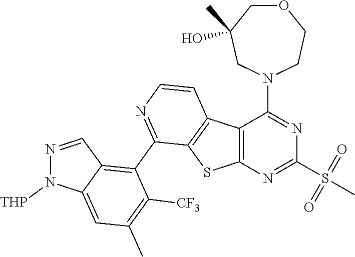 | (6S)-6-methyl-4-(8-(6- methyl-1-(tetrahydro- 2H-pyran-2-yl)-5- (trifluoromethyl)-1H- indazol-4-yl)-2- (methylsulfonyl)pyrido [4′,3′:4,5]thieno[2,3- d]pyrimidin-4-yl)-1,4- oxazepan-6-ol | 677 |
| Int- 160 | 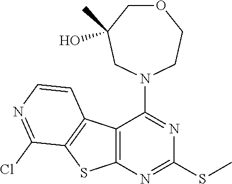 [SMILES: CSc1nc(N2CCOC[C@@](C)(O)C2)c2c(n1)sc1c(Cl)nccc12] Int-143J | 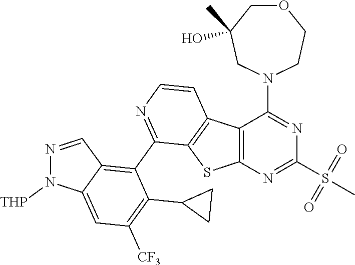 | (6S)-4-(8-(5- cyclopropyl-1- (tetrahydro-2H-pyran-2- yl)-6-(trifluoromethyl)- 1H-indazol-4-yl)-2- (methylsulfonyl)pyrido [4′,3′:4,5]thieno[2,3- d]pyrimidin-4-yl)-6- methyl-1,4-oxazepan-6- ol | 703 |
| Int- 161 | 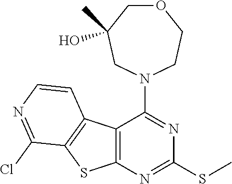 [SMILES: CSc1nc(N2CCOC[C@@](C)(O)C2)c2c(n1)sc1c(Cl)nccc12] Int-143J | 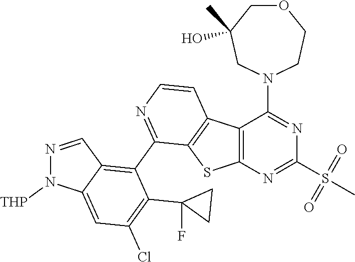 | (6S)-4-(8-(6-chloro-5- (1-fluorocyclopropyl)- 1-(tetrahydro-2H-pyran- 2-yl)-1H-indazol-4-yl)- 2- (methylsulfonyl)pyrido [4′,3′:4,5]thieno[2,3- d]pyrimidin-4-yl)-6- methyl-1,4-oxazepan-6- ol | 687 |

### Intermediate 162: (S)-4-(8-chloro-2-(((2R,7aS)-2-fluorotetrahydro-1H-pyrrolizin-7a(5H)-yl)methoxy)pyrimido[5′,4′:4,5]thieno[2,3-d]pyridazin-4-yl)-6-methyl-1,4-oxazepan-6-ol (Int-162)

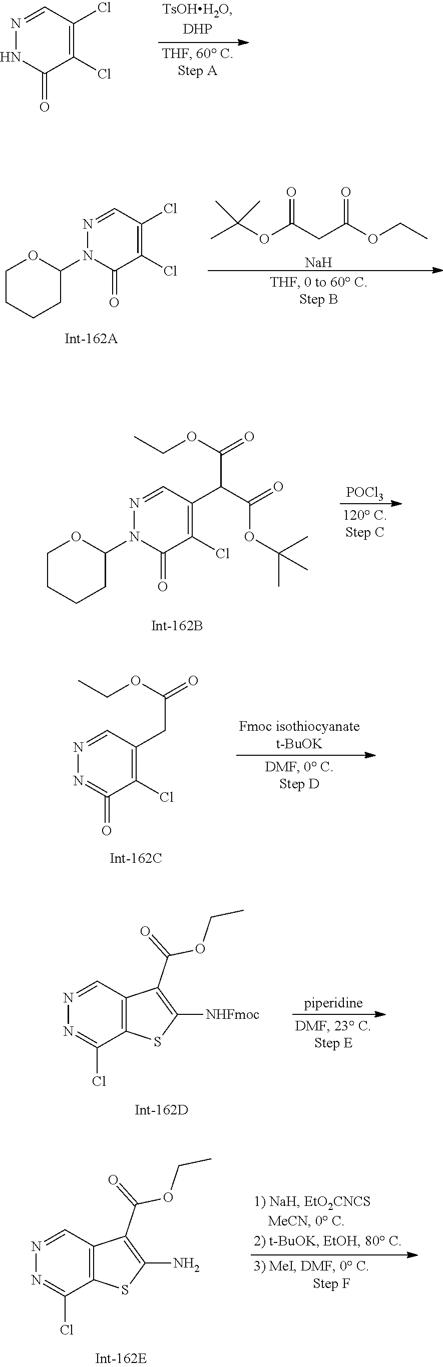

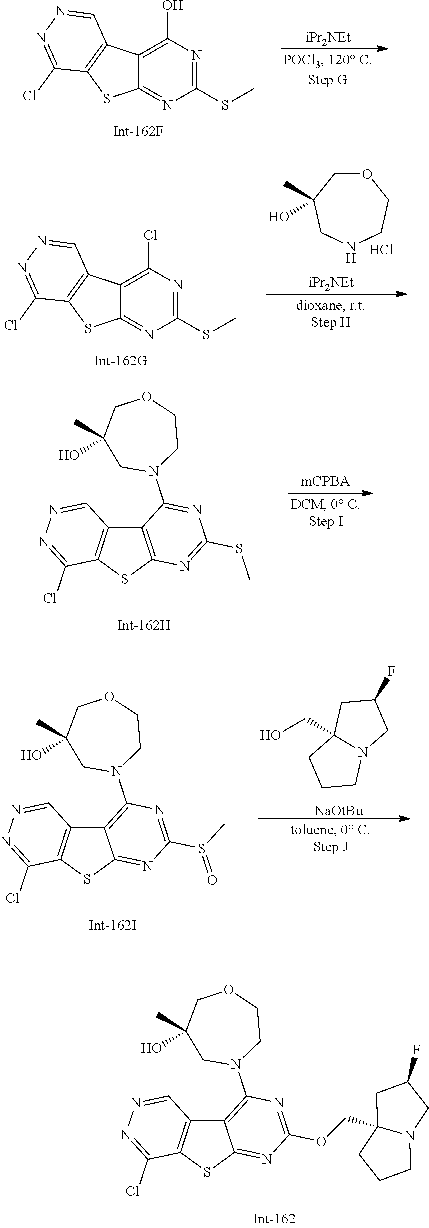

### Step A: 4,5-dichloro-2-(tetrahydro-2H-pyran-2-yl)pyridazin-3(2H)-one (Int-162A)

A 100 mL round bottom flask with a stir bar was charged with 4,5-dichloropyridazin-3 (2H)-one (5.00 g, 30 mmol), TsOH-H₂O (1.15 g, 6.1 mmol), DHP (4.10 mL, 45.5 mmol) and THF (30 mL). The reaction mixture was stirred at 60° C. for 11 h. The reaction mixture was cooled to r.t. and then evaporated. To the residue was added EtOAc, and the reaction mixture was washed with water (50 mL) and brine (50 mL), dried over sodium sulfate, filtered and concentrated under reduced pressure. The residue was purified by silica gel chromatography (10% EtOAc in hexanes) to give 4,5-dichloro-2-(tetrahydro-2H-pyran-2-yl)pyridazin-3 (2H)-one (Int-162A). MS (ESI): m/z (M+H)⁺ 249, 251. ¹H NMR (400 MHz, CDCl₃) δ 7.84 (s, 1H), 4.15-4.11 (m, 1H), 3.78-3.72 (m, 2H), 2.08-2.00 (m, 2H), 1.78-1.67 (m, 4H).

### Step B: 1-(ter-butyl) 3-ethyl 2-(5-chloro-6-oxo-1-(tetrahydro-2H-pyran-2-yl)-1,6-dihydropyridazin-4-yl) malonate (Int-162B)

A 200 mL round bottom flask with a stir bar was charged with NaH (1.40 g, 34.9 mmol, 60 wt % in mineral oil) and THF (30 mL). The reaction mixture was cooled to 0° C. To this mixture was added dropwise tert-butyl ethyl malonate (3.97 mL, 21 mmol) at 0° C. After stirring this mixture at 0° C. for 10 min, a solution of 4,5-dichloro-2-(tetrahydro-2H-pyran-2-yl)pyridazin-3 (2H)-one (Int-162A) (4.35 g, 17.5 mmol) in THF (20 mL) was added to the reaction mixture at 0° C. The reaction mixture was stirred at 0° C. for 5 min, and then stirred at 60° C. for 15 min. The reaction mixture was cooled to r.t. and quenched by the addition of H₂O and 2 M aq. HCl (35 mL). The reaction mixture was extracted with EtOAc (2×50 mL). The combined organic layer was washed with brine (20 mL), dried over sodium sulfate, filtered, and concentrated under reduced pressure. The residue was purified by silica gel chromatography (30% EtOAc in hexanes) to give 1-(tert-butyl) 3-ethyl 2-(5-chloro-6-oxo-1-(tetrahydro-2H-pyran-2-yl)-1,6-dihydropyridazin-4-yl) malonate (Int-162B). MS (ESI): m/z (M+H)⁺ 401, 403.

### Step C: ethyl 2-(5,6-dichloropyridazin-4-yl)acetate (Int-162C)

A 500 mL round bottom flask with a stir bar was charged with 1-(tert-butyl) 3-ethyl 2-(5-chloro-6-oxo-1-(tetrahydro-2H-pyran-2-yl)-1,6-dihydropyridazin-4-yl) malonate (Int-162B) (7.79 g, 17.8 mmol) and phosphoryl chloride (18 mL). The reaction mixture was stirred at 120° C. for 20 min. The reaction mixture was cooled to r.t. and evaporated. To this residue was added toluene, and the mixture was evaporated. EtOAc was added, and the organic layer was washed with water, sat. aq. NaHCO₃, and brine (20 mL each). The organic layer was dried over sodium sulfate, filtered, and concentrated under reduced pressure. The residue was purified by silica gel chromatography (0 to 50% EtOAc in hexanes) to give ethyl 2-(5,6-dichloropyridazin-4-yl)acetate (Int-162C). MS (ESI): m/z (M+H) 235, 237.

### Step D: ethyl 2-((((9H-fluoren-9-yl)methoxy)carbonyl)amino)-7-chlorothieno[2,3-d]pyridazine-3-carboxylate (Int-162D)

A 100 mL round bottom flask with a stir bar was charged with ethyl 2-(5,6-dichloropyridazin-4-yl)acetate (Int-162C) (649 mg, 2.76 mmol) and DMF (9 mL). The mixture was cooled to 0° C. and potassium tert-butoxide (326 mg, 2.90 mmol) was added portionwise. The reaction mixture was stirred at 0° C. for 5 min, and then Fmoc isothiocyanate (816 mg, 2.90 mmol) was added at 0° C. The reaction mixture was stirred at r.t. for 3 min. H₂O was added slowly with stirring. The resulting precipitate was collected by filtration, rinsed with H₂O and hexane, and dried under reduced pressure to give ethyl 2-((((9H-fluoren-9-yl)methoxy) carbonyl)amino)-7-chlorothieno[2,3-d]pyridazine-3-carboxylate (Int-162D). MS (ESI): m/z (M+H)⁺ 480, 482.

### Step E: ethyl 2-amino-7-chlorothieno[2,3-d]pyridazine-3-carboxylate (Int-162E)

A 20 mL round bottom flask with a stir bar was charged with ethyl 2-((((9H-fluoren-9-yl)methoxy) carbonyl)amino)-7-chlorothieno[2,3-d]pyridazine-3-carboxylate (Int-162D) (100 mg, 0.21 mmol), piperidine (45 μL, 0.46 mmol), and DMF (0.70 mL). The reaction mixture was stirred at r.t. for 15 min. H₂O was added slowly with stirring. The resulting precipitate was collected by filtration, rinsed with H₂O and hexane, and dried under reduced pressure to give ethyl 2-amino-7-chlorothieno[2,3-d]pyridazine-3-carboxylate (Int-162E). MS (ESI): m/z (M+H)⁺ 258, 260.

### Step F: 8-chloro-2-(methylthio)pyrimido[5′,4′:4,5]thieno[2,3-d]pyridazin-4-ol (Int-162F)

A 50 mL round bottom flask with a stir bar was charged with ethyl 2-amino-7-chlorothieno[2,3-d]pyridazine-3-carboxylate (Int-162E) (115 mg, 0.45 mmol) and MeCN (9.0 mL). The reaction mixture was cooled to 0° C. To this mixture was added NaH (21 mg, 0.53 mmol, 60 wt % in mineral oil) at 0° C. After stirring the mixture at 0° C. for 5 min. ethoxycarbonyl isothiocyanate (79 μL, 0.67 mmol) was added to the reaction mixture at 0° C. After the reaction mixture was stirred at 0° C. for 90 min, MeOH was added and the reaction mixture was evaporated. To a residue was added potassium tert-butoxide (150 mg, 1.3 mmol) and EtOH (9.0 mL), and then the reaction mixture was stirred at 80° C. for 1 h. After the reaction mixture was cooled to r.t., the reaction mixture was evaporated. To a residue was added DMF (4.4 mL). After the reaction mixture was cooled to 0° C., MeI (31 μL, 0.49 mmol) was added. The reaction mixture was stirred at 0° C. for 5 min. H₂O and 2 M. HCl (1.4 mL) were added slowly with stirring. The resulting precipitate was collected by filtration, rinsed with H₂O and hexane, and dried under reduced pressure to give 8-chloro-2-(methylthio)pyrimido[5′,4′:4,5]thieno[2,3-d]pyridazin-4-ol (Int-162F), which may exist in a tautomeric form. MS (ESI): m/z (M+H)⁺ 285, 287.

### Step G: 4,8-dichloro-2-(methylthio)pyrimido[5′,4′:4,5]thieno[2,3-d]pyridazine (Int-162G)

A 20 mL round bottom flask with a stir bar was charged with 8-chloro-2-(methylthio)pyrimido[5′,4′:4,5]thieno[2,3-d]pyridazin-4-ol (Int-162F) (80 mg, 0.28 mmol), phosphoryl chloride (0.94 mL) and DIPEA (0.096 mL, 0.56 mmol). The reaction mixture was stirred at 120° C. for 5 min. The reaction mixture was cooled to r.t. and evaporated. To this residue was added water and CHCl₃. The layers were separated and the aqueous phase was extracted with CHCl₃ (3×10 mL). The combined organic layer was washed with brine (10 mL), dried over sodium sulfate, filtered, and concentrated under reduced pressure to give 4,8-dichloro-2-(methylthio)pyrimido[5′,4′:4,5]thieno[2,3-d]pyridazine (Int-162G). MS (ESI): m/z (M+H)⁺ 303, 305. ¹H NMR (400 MHz, CDCl₃) δ 10.13 (s, 1H), 2.72 (s, 3H).

### Step H: (S)-4-(8-chloro-2-(methylthio)pyrimido[5′,4′:4,5]thieno[2,3-d]pyridazin-4-yl)-6-methyl-1,4-oxazepan-6-ol (Int-162H)

To a stirred mixture of 4,8-dichloro-2-(methylthio)pyrimido[5′,4′:4,5]thieno[2,3-d]pyridazine (Int-162G) (800 mg, 2.64 mmol) and N,N-diisopropylethylamine (2.30 mL, 13.2 mmol) in 1,4-dioxane (26 mL) was added (S)-6-methyl-1,4-oxazepan-6-ol hydrochloride (487 mg, 2.90 mmol) at r.t. After being stirred for 15 min, to the mixture was added water. The resulting precipitate was filtered and washed with water. The filtered solid was dried under vacuum at 40° C. to give(S)-4-(8-chloro-2-(methylthio)pyrimido[5′,4′:4,5]thieno[2,3-d]pyridazin-4-yl)-6-methyl-1,4-oxazepan-6-ol (Int-162H). MS (ESI): m/z (M+H)⁺ 398, 400.

### Step I: (6S)-4-(8-chloro-2-(methylsulfinyl)pyrimido[5′,4′:4,5]thieno[2,3-d]pyridazin-4-yl)-6-methyl-1,4-oxazepan-6-ol (Int-1621)

To a stirred solution of(S)-4-(8-chloro-2-(methylthio)pyrimido[5′,4′:4,5]thieno[2,3-d]pyridazin-4-yl)-6-methyl-1,4-oxazepan-6-ol (Int-162H) (872 mg, 2.19 mmol) in DCM (22 mL) was added mCPBA (504 mg, 2.19 mmol, 75 wt %) at 0° C. After 5 min, to the mixture was added sat. aq. sodium bicarbonate and the mixture was extracted with chloroform. The organic extract was washed with brine, dried over sodium sulfate, filtered, and concentrated to give (6S)-4-(8-chloro-2-(methylsulfinyl)pyrimido[5′,4′:4,5]thieno[2,3-d]pyridazin-4-yl)-6-methyl-1,4-oxazepan-6-ol (Int-1621). The crude material was used directly in the next step without further purification. MS (ESI): m/z (M+H)⁺ 414, 416.

### Step J: (S)-4-(8-chloro-2-(((2R,7aS)-2-fluorotetrahydro-1H-pyrrolizin-7a(5H)-yl)methoxy)pyrimido[5′4′:4,5]thieno[2,3-d]pyridazin-4-yl)-6-methyl-1,4-oxazepan-6-ol (Int-162)

To a mixture of ((2R,7aS)-2-fluorotetrahydro-1H-pyrrolizin-7a(5H)-yl) methanol (516 mg, 3.24 mmol) and (6S)-4-(8-chloro-2-(methylsulfinyl)pyrimido[5′,4′:4,5]thieno[2,3-d]pyridazin-4-yl)-6-methyl-1,4-oxazepan-6-ol (Int-321) (894 mg, 2.16 mmol) was added toluene (43 mL). After the mixture was cooled 0° C., sodium tert-butoxide (623 mg, 6.48 mmol) was added and stirred at 0° C. After 60 min, water was added to the mixture and the mixture was extracted with EtOAc (3×). The combined organic phase was dried over sodium sulfate, filtered, and concentrated. The crude residue was purified via silica gel chromatography (0 to 20% MeOH in chloroform) to give(S)-4-(8-chloro-2-(((2R,7aS)-2-fluorotetrahydro-1H-pyrrolizin-7a(5H)-yl)methoxy)pyrimido[5′,4′:4,5]thieno[2,3-d]pyridazin-4-yl)-6-methyl-1,4-oxazepan-6-ol (Int-162). MS (ESI): m/z (M+H)⁺ 509, 511. ¹H NMR (400 MHz, CDCl₃) δ=9.35 (s, 1H), 5.79 (br, 1H), 5.35-5.18 (m, 1H), 4.30-4.26 (m, 1H), 4.21-4.18 (m, 1H), 4.13-4.09 (m, 1H), 4.06-4.02 (m, 1H), 3.95-3.91 (m, 1H), 3.85-3.75 (m, 2H), 3.66-3.62 (m, 1H), 3.54-3.49 (m, 1H), 3.34-3.31 (m, 1H), 3.28-3.21 (m, 2H), 3.15-3.12 (m, 1H), 2.99-2.95 (m, 1H), 2.22-2.05 (m, 4H), 1.99-1.85 (m, 2H), 1.33 (s, 3H).

The compounds in the table below were synthesized using a similar procedure as described in the synthesis of Int-162 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int- 163 | 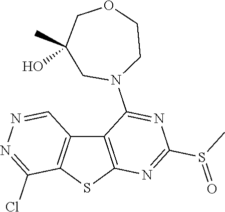 [SMILES: CS(=O)c1nc(N2CCOC[C@@](C)(O)C2)c2c(n1)sc1c(Cl)nncc12] | 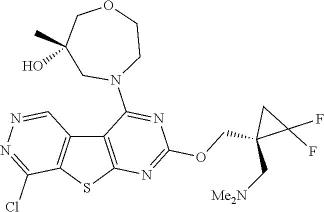 [SMILES: CN(C)C[C@@]1(COc2nc(N3CCOC[C@@](C)(O)C3)c3c(n2)sc2c(Cl)nncc23)CC1(F)F] | (S)-4-(8-chloro-2-(((R)-1- ((dimethylamino)methyl)-2,2- difluorocyclopropyl)methoxy) pyrimido[5′,4′:4,5]thieno[2,3- d]pyridazin-4-yl)-6-methyl- 1,4-oxazepan-6-ol | 515 |
| | Int-162I | | | |
| Int- 164 | 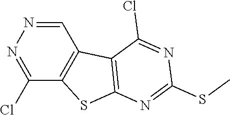 [SMILES: CSc1nc(Cl)c2c(n1)sc1c(Cl)nncc12] Int-162G | 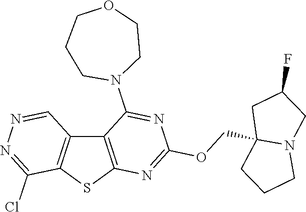 [SMILES: F[C@H]1CN2CCC[C@@]2(COc2nc(N3CCCOCC3)c3c(n2)sc2c(Cl)nncc23)C1] | 4-(8-chloro-2-(((2R,7aS)-2- fluorotetrahydro-1H- pyrrolizin-7a(5H)- yl)methoxy)pyrimido[5′,4′:4,5] thieno[2,3-d]pyridazin-4-yl)- 1,4-oxazepane | 479 |

### Intermediate 165: (S)-4-(8-chloro-2-(((2R,7aS)-2-fluorotetrahydro-1H-pyrrolizin-7a(5H)-yl)methoxy)pyrido[4′,3′:4,5]furo[2,3-d]pyrimidin-4-yl)-6-methyl-1,4-oxazepam-6-ol (Int-165)

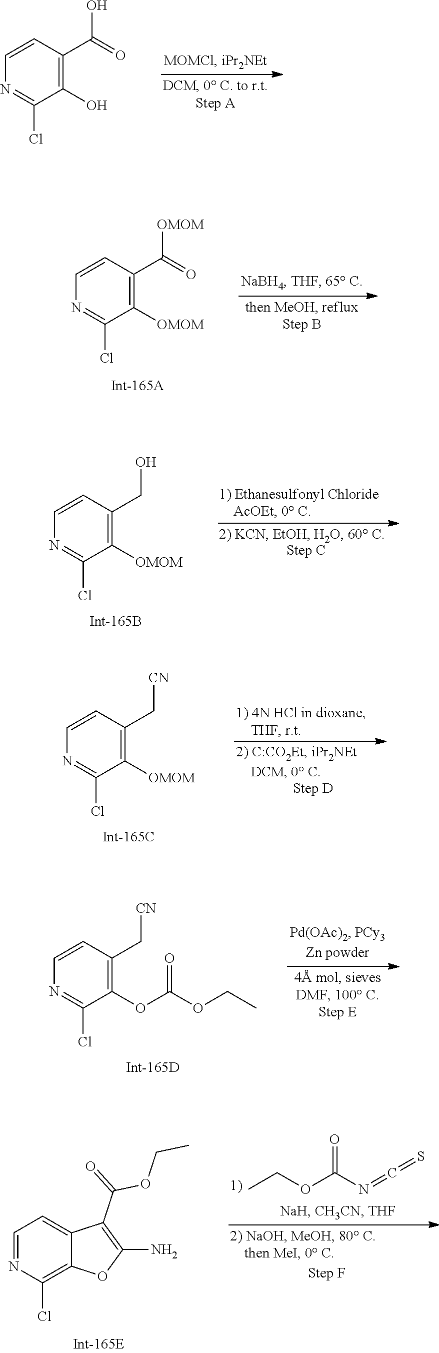

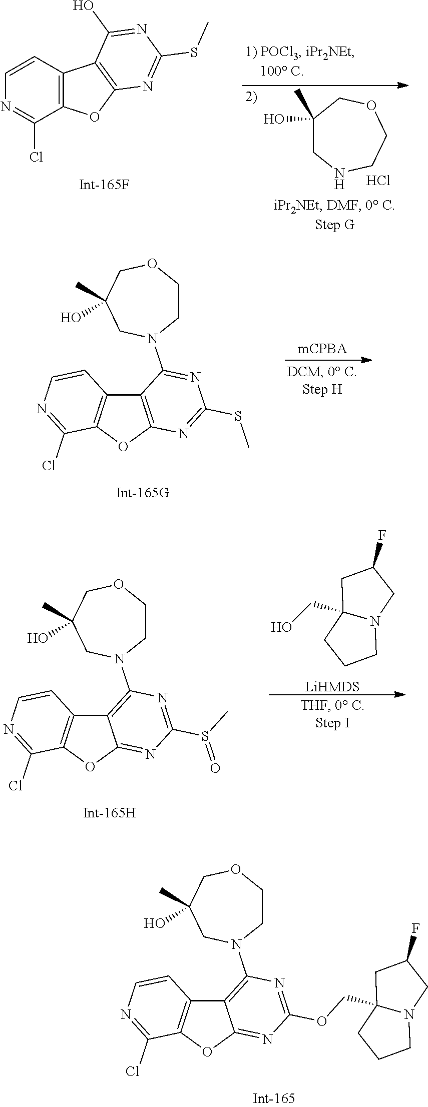

### Step A: Methoxymethyl 2-chloro-3-(methoxymethoxy) isonicotinate (Int-165A)

To a stirred mixture of 2-chloro-3-hydroxyisonicotinic acid (5.0 g, 29 mmol) and N,N-diisopropylethylamine (25 mL, 140 mmol) in DCM (150 mL) was added chloro(methoxy) methane (7.7 mL, 100 mmol) at 0° C. The reaction was allowed to warm to r.t. over 3 h. After 3 h, the reaction was quenched with water and the mixture was extracted with chloroform. The organic phase was washed with brine, dried over sodium sulfate, filtered, and concentrated. The crude residue was purified via silica gel chromatography (15% to 30% EtOAc in hexane) to give methoxymethyl 2-chloro-3-(methoxymethoxy) isonicotinate (Int-165A). MS (ESI): m/z (M+H)⁺ 262. ¹H NMR (400 MHz, CDCl₃) δ 8.29 (d, J=5.0 Hz, 1H), 7.60 (d. J=5.0 Hz, 1H), 5.51 (s, 2H), 5.23 (s, 2H), 3.65 (s, 3H), 3.59 (s, 3H).

### Step B: (2-chloro-3-(methoxymethoxy)pyridin-4-yl) methanol (Int-165B)

To a stirred solution of methoxymethyl 2-chloro-3-(methoxymethoxy) isonicotinate (Int-165A) (5.70 g, 22.0 mmol) in THF (109 mL) was added sodium borohydride (7.02 g, 185 mmol) at 65° C. in a portionwise manner. After addition, to the mixture was added MeOH (110 ml) dropwise, then the mixture was warmed to 90° C. and stirred for 0.5 h. After cooling, the reaction was quenched with sat. aq. ammonium chloride and concentrated in vacuo. To the residue was added EtOAc and the mixture was washed with brine, dried over sodium sulfate, filtered, and concentrated to give (2-chloro-3-(methoxymethoxy)pyridin-4-yl) methanol (Int-165B). MS (ESI): m/z (M+H)⁺ 204. ¹H NMR (400 MHz, CDCl₃) δ 8.22 (d, J=4.8 Hz, 1H), 7.36 (d, J=4.8 Hz, 1H), 5.18 (s, 2H), 4.75 (s, 2H), 3.67 (s, 3H), 2.81 (br s, 1H).

### Step C: 2-(2-chloro-3-(methoxymethoxy)pyridin-4-yl) acetonitrile (Int-165C)

To a stirred solution of (2-chloro-3-(methoxymethoxy)pyridin-4-yl) methanol (Int-165B) (3.53 g, 17.3 mmol) and N,N-diisopropylethylamine (5.93 mmol, 34.7 mmol) in EtOAc (87 mL) was added ethanesulfonyl chloride (2.46 mmol, 26 mmol) at 0° C. The mixture was stirred at 0° C. for 0.5 h. Then the mixture was diluted with EtOAc and quenched with water. The organic phase was washed with water and brine, dried over sodium sulfate, filtered, and concentrated to give a corresponding ethanesulfonyl compound which was used directly in the next step without further purification. The crude ethanesulfonyl compound in ethanol (43 mL) was added to a stirred solution of potassium cyanide (2.26 g, 34.7 mmol) in water (43 mL) at 60° C. Then the mixture was stirred at 60° C. for 1.5 h. After cooling, to the reaction was added EtOAc and the mixture was washed with brine, dried over sodium sulfate, filtered, and concentrated. The crude residue was purified via silica gel chromatography (15% to 48% EtOAc in hexane) to give 2-(2-chloro-3-(methoxymethoxy)pyridin-4-yl) acetonitrile (Int-165C). MS (ESI): m/z (M+H)⁺ 213. ¹H NMR (400 MHz, CDCl₃) δ 8.26 (d, J=4.9 Hz, 1H), 7.43 (d, J=4.9 Hz, 1H), 5.20 (s, 2H), 3.96 (s, 2H), 3.63 (s, 3H).

### Step D: 2-chloro-4-(cyanomethyl)pyridin-3-yl ethyl carbonate (Int-165D)

To a stirred suspension of 2-(2-chloro-3-(methoxymethoxy)pyridin-4-yl) acetonitrile (Int-165C) (1.2 g, 5.6 mmol) in THF (2.5 mL) was added HCl (5 mL, 4 N in dioxane) at r.t. After being stirred for 1 h, the mixture was concentrated in vacuo. To the residue was added DCM (20 ml) and N,N-diisopropylethylamine (4.8 mL, 28 mmol) and then cooled to 0° C. To a stirred this mixture was added ethyl chloroformate (0.65 mL, 6.8 mmol) and allowed to stir at 0° C. for 0.5 h. The reaction was quenched with water and the mixture was extracted with chloroform. The organic phase was washed with brine, dried over sodium sulfate, filtered, and concentrated. The crude residue was purified via silica gel chromatography (15 to 55% EtOAc in hexane) to give 2-chloro-4-(cyanomethyl)pyridin-3-yl ethyl carbonate (Int-165D). MS (ESI): m/z (M+H)⁺ 241. ¹H NMR (400 MHz, CDCl₃) δ 8.39 (d, J=5.0 Hz, 1H), 7.48 (d, J=5.0 Hz, 1H), 4.43 (q, J=7.1 Hz, 2H), 3.81 (s, 2H), 1.45 (t, J=7.1 Hz, 3H).

### Step E: Ethyl 2-amino-7-chlorofuro[2,3-c]pyridine-3-carboxylate (Int-165E)

A mixture of palladium acetate (75.0 mg, 0.332 mmol), tricyclohexylphosphine (186 mg, 0.665 mmol), zinc (45.0 mg, 0.665 mmol), and powdered 4 Å molecular sieves (350 mg) in DMF (24 mL) was degassed 3 times and stirred at r.t. for 15 min. Then to the mixture was added 2-chloro-4-(cyanomethyl)pyridin-3-yl ethyl carbonate (Int-165D) (800 mg, 3.32 mmol) in DMF (12 mL) and the mixture was heated at 100° C. for 9 h. After cooling, the mixture was filtered through a CELITE® pad and the filtrate was partitioned between EtOAc and water. The organic phase was washed with water and brine, dried over sodium sulfate, filtered, and concentrated. The crude residue was purified via silica gel chromatography (20 to 60% EtOAc in hexane) to give ethyl 2-amino-7-chlorofuro[2,3-c]pyridine-3-carboxylate (Int-165E). MS (ESI): m/z (M+H)⁺ 241. ¹H NMR (400 MHz, CDCl₃) δ 8.16 (d, J=5.1 Hz, 1H), 7.52 (d, J=5.1 Hz, 1H), 6.34 (br s, 2H), 4.41 (q, J=7.1 Hz, 2H), 1.46 (t, J=7.1 Hz, 3H).

### Step F: 8-chloro-2-(methylthio)pyrido[4′,3′:4,5]furo[2,3-d]pyrimidin-4-ol (Int-165F)

To a stirred solution of ethyl 2-amino-7-chlorofuro[2,3-c]pyridine-3-carboxylate (Int-165E) (80 mg, 0.332 mmol) and ethoxycarbonyl isothiocyanate (58 μL, 0.499 mmol) in acetonitrile (3 mL) and THF (1 ml) was added sodium hydride (17 mg, 0.432 mmol, 60 wt % in mineral oil) at r.t. After being stirred for 0.5 h, the volatile materials were removed by evaporation. To the residue was added EtOAc and the resulting precipitate was collected by filtration to give a thiourea compound which was used directly in the next step without further purification. A mixture of the thiourea compound in 1 M aq. NaOH (1.6 mL), water (0.8 mL) and methanol (1.6 mL) was stirred at 80° C. for 1.5 h. Then the mixture was cooled to 0° C. and methyl iodide (25 μL, 0.399 mmol) was added to the mixture. The reaction mixture was stirred for 0.5 h at 0° C., and then acidified to pH 3 by dilute hydrochloric acid. The resulting precipitate was collected by filtration to 8-chloro-2-(methylthio)pyrido[4′,3′:4,5]furo[2,3-d]pyrimidin-4-ol (Int-165F), which may exist in a tautomeric form. MS (ESI): m/z (M+H)⁺ 268. ¹H NMR (400 MHz, DMSO-d₆) δ 13.52 (br s, 1H), 8.35 (d, J=5.0 Hz, 1H), 7.82 (d, J=5.0 Hz, 1H), 2.64 (s, 3H).

### Step G: (S)-4-(8-chloro-2-(methylthio)pyrido[4′,3′:4,5]furo[2,3-d]pyrimidin-4-yl)-6-methyl-1,4-oxazepan-6-ol (Int-165G)

A mixture of 8-chloro-2-(methylthio)pyrido[4′,3′:4,5]furo[2,3-d]pyrimidin-4-ol (Int-165F) (114 mg, 0.426 mmol), phosphorus oxychloride (2 mL, 21.4 mmol) and N,N-diisopropylethylamine (146 μL, 0.851 mmol) was stirred at 100° C. for 1.5 h. Then volatile materials were removed by evaporation and the residue was partitioned between EtOAc and sat. aq. sodium bicarbonate. The organic phase was washed with brine, dried over sodium sulfate, filtered, and concentrated to give the desired dichloride compound which was used directly in the next step without further purification. To the stirred crude dichloride compound and N,N-diisopropylethylamine (732 mL, 4.26 mmol) in DMF (5 mL) was added (S)-6-methyl-1,4-oxazepan-6-ol hydrochloride (107 mg, 0.638 mmol) at 0° C. The reaction mixture was allowed to stir to r.t. for 0.5 h, then partitioned between EtOAc and water. The organic phase was washed with water and brine, dried over sodium sulfate, filtered, and concentrated. The crude residue was purified via silica gel chromatography (0% to 5% MeOH in EtOAc) to give(S)-4-(8-chloro-2-(methylthio)pyrido[4′,3′:4,5]furo[2,3-d]pyrimidin-4-yl)-6-methyl-1,4-oxazepan-6-ol (Int-165G). MS (ESI): m/z (M+H)⁺ 381. ¹H NMR (400 MHz, CDCl₃) δ 8.37 (d, J=5.4 Hz, 1H), 7.58 (d, J=5.4 Hz, 1H), 4.97-4.83 (m, 1H), 4.48-4.38 (m, 2H), 4.06-3.98 (m, 1H), 3.90-3.79 (m, 2H), 3.75 (d, J=12.6 Hz, 1H), 3.58 (d, J=14.6 Hz, 1H), 3.49 (d, J=12.3 Hz, 1H), 2.66 (s, 3H), 1.36 (s, 3H).

### Step H: (6S)-4-(8-chloro-2-(methylsulfinyl)pyrido[4′,3′:4,5]furo[2,3-d]pyrimidin-4-yl)-6-methyl-1,4-oxazepan-6-ol (Int-165H)

To a stirred solution of(S)-4-(8-chloro-2-(methylthio)pyrido[4′,3′:4,5]furo[2,3-d]pyrimidin-4-yl)-6-methyl-1,4-oxazepan-6-ol (Int-165G) (130 mg, 0.341 mmol) in DCM (5 mL) was added mCPBA (79 mg, 0.341 mmol, 75 wt %) at 0° C. After 30 min, to the mixture was added sat. aq. sodium bicarbonate and the mixture was extracted with chloroform. The organic extract was washed with brine, dried over sodium sulfate, filtered, and concentrated to give (6S)-4-(8-chloro-2-(methylsulfinyl)pyrido[4′,3′:4,5]furo[2,3-d]pyrimidin-4-yl)-6-methyl-1,4-oxazepan-6-ol (Int-165H). The crude material was used directly in the next step without further purification. MS (ESI): m/z (M+H)⁺ 397.

### Step I: (S)-4-(18-chloro-2-((2R,7aS)-2-fluorotetrahydro-1H-pyrrolizin-7a(5H)-yl)methoxy)pyrido[4′,3′:4,5]furo[2,3-d]pyrimidin-4-yl)-6-methyl-1,4-oxazepan-6-ol (Int-165)

To a stirred solution of ((2R,7aS)-2-fluorotetrahydro-1H-pyrrolizin-7a(5H)-yl) methanol (65 mg, 0.41 mmol) in THF (25 mL) was added lithium bis(trimethylsilyl)amide (690 μL, 0.69 mmol, 1.0 M THF solution) at 0° C. and stirred at 0° C. for 15 min. Then to the mixture was added (6S)-4-(8-chloro-2-(methylsulfinyl)pyrido[4′,3′:4,5]furo[2,3-d]pyrimidin-4-yl)-6-methyl-1,4-oxazepan-6-ol (Int-165H) (108 mg, 0.27 mmol) in THF (5 mL) dropwise. The mixture was allowed to stir at 0° C. for 30 min and the mixture was partitioned between EtOAc and water. The combined organic phase was dried over sodium sulfate, filtered, and concentrated. The crude residue was purified via silica gel chromatography (0 to 30% MeOH in chloroform) to give(S)-4-(8-chloro-2-(((2R,7aS)-2-fluorotetrahydro-1H-pyrrolizin-7a(5H)-yl)methoxy)pyrido[4′,3′:4,5]furo[2,3-d]pyrimidin-4-yl)-6-methyl-1,4-oxazepan-6-ol (Int-165). MS (ESI): m/z (M+H)⁺ 492. ¹H NMR (400 MHz, CDCl₃) δ 8.35 (d, J=5.5 Hz, 1H), 7.53 (d, J=5.5 Hz, 1H), 5.62-5.42 (m, 1H), 5.40-5.17 (m, 1H), 4.48-4.40 (m, 1H), 4.38-4.26 (m, 2H), 4.27-4.19 (m, 1H), 4.05-3.95 (m, 1H), 3.85-3.70 (m, 3H), 3.55 (d, J=14.8 Hz, 1H), 3.44 (d, J=12.5 Hz, 1H), 3.37-3.13 (m, 3H), 3.07-2.91 (m, 1H), 2.28-2.14 (m, 2H), 2.11-2.05 (m, 1H), 2.02-1.83 (m, 3H), 1.36 (s, 3H).

The compound in the table below was synthesized using a similar procedure as described in the synthesis of Int-165 by making the appropriate substitutions for starting material, intermediates, and/or reagents. Such starting materials, intermediates, and/or reagents are available commercially, synthesized as described in the literature, synthesized using methods available to those skilled in the art or synthesized as described herein

| | Starting | | | [M + H]⁺ |
|---|---|---|---|---|
| Int. | Material | Structure | Compound Name | Found |
| Int- 166 | 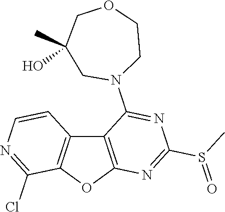 [SMILES: CS(=O)c1nc(N2CCOC[C@@](C)(O)C2)c2c(n1)oc1c(Cl)nccc12] | 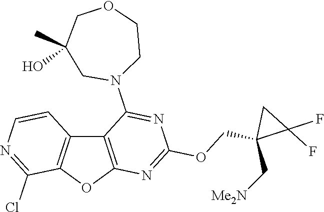 [SMILES: CN(C)C[C@@]1(COc2nc(N3CCOC[C@@](C)(O)C3)c3c(n2)oc2c(Cl)nccc23)CC1(F)F] | (S)-4-(8-chloro-2-(((R)-1- ((dimethylamino)methyl)- 2,2- difluorocyclopropyl)methoxy) pyrido[4′,3′:4,5]furo[2,3- d]pyrimidin-4-yl)-6-methyl- 1,4-oxazepan-6-ol | 498 |
| | Int-165H | | | |

### Intermediate 167: (6S)-4-(2-chloro-9-(5-cyclopropyl-6-methyl-1-(tetrahydro-2H-pyran-2-yl)-1H-indazol-4-yl)pyrazino[1′,2′:1,5]pyrrolo[3,2-d]pyrimidin-4-yl)-6-methyl-1,4-oxazepan-6-ol (Int-167)

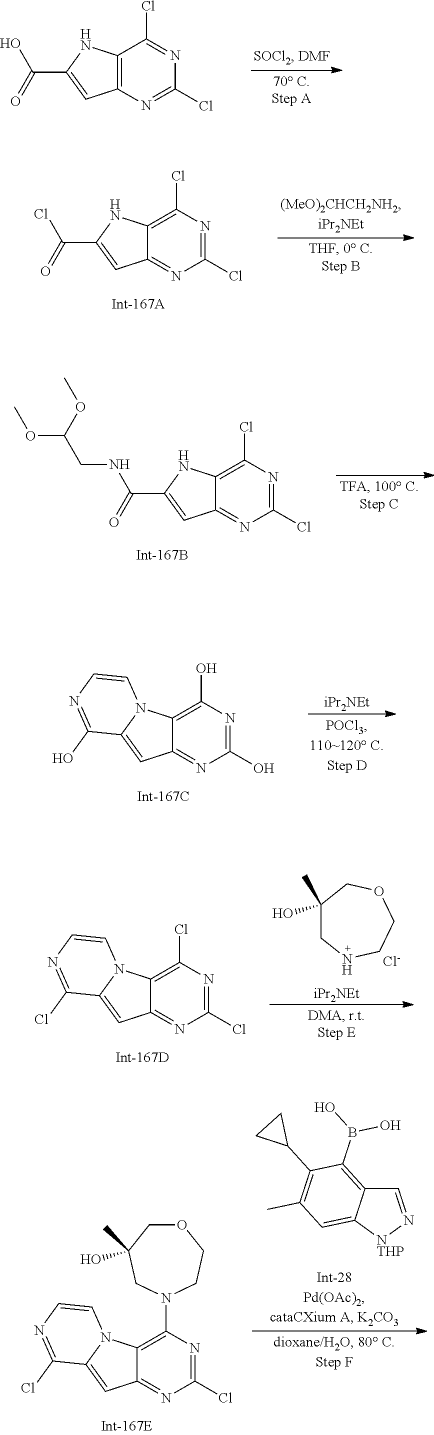

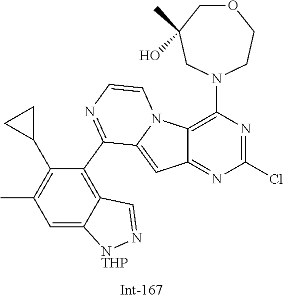

### Step A: 2,4-dichloro-5H-pyrrolo[3,2-d]pyrimidine-6-carbonyl chloride (Int-167A)

A 100 mL round-bottom flask with a stir bar was charged with 2,4-dichloro-5H-pyrrolo[3,2-d]pyrimidine-6-carboxylic acid (2.00 g, 8.62 mmol), SOCl₂ (6.20 mL, 86.2 mmol), and DMF (10 μL). The reaction mixture was stirred at 70° C. for 90 min. The reaction mixture was cooled to room temperature and concentrated in vacuo to provide 2,4-dichloro-5H-pyrrolo[3,2-d]pyrimidine-6-carbonyl chloride (Int-167A), which was used directly in the next step without further purification.

### Step B: 2,4-dichloro-N-(2,2-dimethoxyethyl)-5H-pyrrolo[3,2-d]pyrimidine-6-carboxamide (Int-167B)

To 2,4-dichloro-5H-pyrrolo[3,2-d]pyrimidine-6-carbonyl chloride (2.16 g, 8.62 mmol) (Int-167A) was added THF (17.0 mL) and the mixture was cooled to 0° C. After stirring the mixture at 0° C. for 10 min, aminoacetaldehyde dimethyl acetal (0.92 mL, 8.45 mmol) and N,N-diisopropylethylamine (4.42 mL, 25.7 mmol) were added at 0° C. The reaction mixture was stirred at 0° C. for 5 min. The reaction mixture was quenched by the addition of H₂O (50 mL) and the reaction mixture was extracted with EtOAc (3×50 mL). The combined organic layers were washed with brine (20 mL), dried over sodium sulfate, filtered, and concentrated under reduced pressure to provide 2,4-dichloro-N-(2,2-dimethoxyethyl)-5H-pyrrolo[3,2-d]pyrimidine-6-carboxamide (Int-167B), which was used directly in the next step without further purification.

### Step C: Pyrazino[1′,2′:1,5]pyrrolo[3,2-d]pyrimidine-2,4,9-triol (Int-167C)

To 2,4-dichloro-N-(2,2-dimethoxyethyl)-5H-pyrrolo[3,2-d]pyrimidine-6-carboxamide (Int-167B) (2.75 g, 8.62 mmol) was added TFA (23.0 mL), and the reaction mixture was stirred at 100° C. for 1 h. The reaction mixture was cooled to 0° C. MeOH was added slowly with stirring at 0° C. The resulting precipitate was collected by filtration, rinsed with hexane, and dried under reduced pressure to give pyrazino[1′,2′:1,5]pyrrolo[3,2-d]pyrimidine-2,4,9-triol (Int-167C). MS (ESI): m/z (M+H)⁺ 219. ¹H NMR (400 MHz, d⁶-DMSO) δ 11.08 (d, 1H, J=5.2 Hz), 11.02 (s, 1H), 11.00 (s, 1H), 7.75 (d, 1H, J=5.2 Hz), 6.71 (t, 1H, J=5.6 Hz), 6.33 (s, 1H).

### Step D: 2,4,9-trichloropyrazino[1′,2′:1,5]pyrrolo[3,2-d]pyrimidine (Int-167D)

A 50 mL round bottom flask with stir bar was charged with pyrazino[1′,2′:1,5]pyrrolo[3,2-d]pyrimidine-2,4,9-triol (Int-167C) (586 mg, 2.68 mmol), phosphoryl chloride (9.0 mL), and N,N-diisopropylethyamine (2.30 mL, 13.4 mmol). The reaction mixture was stirred at 110° C. for 2.5 h and then 120° C. for 6 h. The reaction mixture was cooled to room temperature and then evaporated. To this residue was added CHCl₃ and water and the reaction mixture was extracted with CHCl₃ (3×50 mL). The combined organic layers were washed with brine (20 mL), dried over Na₂SO₄, filtered, and concentrated under reduced pressure to provide 2,4,9-trichloropyrazino[1′,2′:1,5]pyrrolo[3,2-d]pyrimidine (Int-167D), which was used directly in the next step without further purification.

### Step E: (S)-4-(2,9-dichloropyrazino[1,2′:1,5]pyrrolo[3,2-d]pyrimidin-4-yl)-6-methyl-1,4-oxazepan-6-ol (Int-167E)

A 200 mL round bottom flask with a stir bar was charged with 2,4,9-trichloropyrazino[1′,2′:1,5]pyrrolo[3,2-d]pyrimidine (733 mg, 2.68 mmol) (Int-167D), (S)-6-methyl-1,4-oxazepan-6-ol hydrochloride (496 mg, 2.96 mmol), N,N-diisopropylethyamine (2.30 mL, 13.4 mmol), and DMA (5.4 mL). The reaction mixture was stirred at room temperature for 10 min. The reaction mixture was quenched by the addition of H₂O extracted with AcOEt (3×50 mL), The combined organic layer was washed with brine (20 mL), dried over Na₂SO₄, filtered, and concentrated under reduced pressure. The crude residue was purified via silica gel chromatography (50 to 100% EtOAc in hexanes) to give(S)-4-(2,9-dichloropyrazino[1′,2′:1,5]pyrrolo[3,2-d]pyrimidin-4-yl)-6-methyl-1,4-oxazepam-6-ol (Int-167E), MS (ESD): m/z (M+H)⁺ 368, 370, ¹H NMR (400 MHz, CDCl₃) δ 8.02 (dd, J=0.8, 4.8 Hz, 1H), 7.67 (d. J=4.8 Hz, 1H), 7.22 (d, J=0.8 Hz, 1H), 5.94 (br, 1H), 4.05 (d, J=15 Hz, 1H), 3.97-3.92 (m, 1H), 3.84 (d, J=13 Hz, 1H), 3.75-3.73 (m, 2H), 3.64 (d, J=15 Hz, 1H), 3.49-3.37 (m, 2H), 1.38 (s, 3H).

### Step F: (6S)-4-(2-chloro-9-(5-cyclopropyl-6-methyl-1-(tetrahydro-2H-pyran-2-yl)-1H-indazol-4-yl)pyrazino[1′,2′:1,5]pyrrolo[3,2-d]pyrimidin-4-yl)-6-methyl-1,4-oxazepan-6-ol (Int-167)

A round bottom flask with a stir bar was charged with (S)-4-(2,9-dichloropyrazino[1′,2′:1,5]pyrrolo[3,2-d]pyrimidin-4-yl)-6-methyl-1,4-oxazepan-6-ol (Int-167E) (50.0 mg, 0.136 mmol), Pd(OAc)₂ (9.1 mg, 0.041 mmol), di(1-adamantyl)-n-butylphosphinic (29.2 mg, 0.0815 mmol), K₂CO₃ (0.340 mL, 0.679 mmol, 2 M in H₂O), (5-cyclopropyl-6-methyl-1-(tetrahydro-2H-pyran-2-yl)-1H-indazol-4-yl)boronic acid (Int-28) (65.2 mg, 0.217 mmol), and 1,4-dioxane (1.36 mL). The mixture was degassed with N₂ (3×) and stirred at 80° C. under N₂ for 11 h. After cooling, the mixture was diluted with CHCl₃ and water. The mixture was extracted with CHCl₃ (3×) and the combined organic layers were washed with brine, dried over Na₂SO₄, filtered, and concentrated. The residue was purified via silica gel chromatography (0 to 10% MeOH in CHCl₃) to afford (6S)-4-(2-chloro-9-(5-cyclopropyl-6-methyl-1-(tetrahydro-2H-pyran-2-yl)-1H-indazol-4-yl)pyrazino[1′,2′:1,5]pyrrolo[3,2-d]pyrimidin-4-yl)-6-methyl-1,4-oxazepan-6-ol (Int-167). MS (ESI): m/z (M+H)⁺ 588, 590. ¹H NMR (400 MHz, CDCl₃) δ 8.11-8.06 (m, 2H), 7.72-7.56 (m, 2H), 6.73-6.70 (m, 1H), 6.35-6.26 (m, 1H), 6.78-6.64 (m, 1H), 4.08-4.02 (m, 4H), 3.89-3.76 (m, 4H), 3.44-3.39 (m, 2H), 2.68-2.67 (m, 3H), 2.58-2.52 (m, 1H), 2.19-2.12 (m, 2H), 2.08-2.00 (m, 4H), 1.42-1.41 (m, 3H), 0.82-0.73 (m, 0.5H), 0.68-0.66 (m, 0.5H), 0.64-0.61 (m, 0.5H), 0.37-0.30 (m, 0.5H), 0.19-0.11 (m, 2H).

The compounds in the table below were synthesized using a similar procedure as described in the synthesis of Int-167 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Int. | Material | Structure | Compound Name | Found |
| Int- 168 | 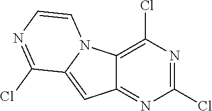 [SMILES: Clc1nc(Cl)c2c(cc3c(Cl)nccn32)n1] Int-167D | 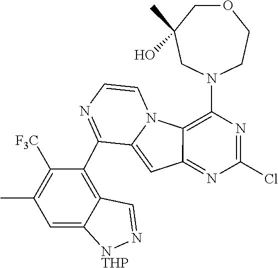 | (6S)-4-(2-chloro-9-(6- methyl-1-(tetrahydro-2H- pyran-2-yl)-5- (trifluoromethyl)-1H- indazol-4- yl)pyrazino[l′,2′:1,5]pyrrolo [3,2-d]pyrimidin-4-yl)-6- methyl-1,4-oxazepan-6-ol | 616 |
| Int- 169 | 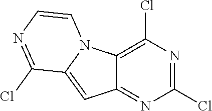 [SMILES: Clc1nc(Cl)c2c(cc3c(Cl)nccn32)n1] Int-167D | 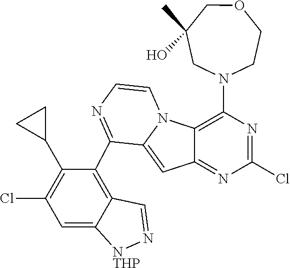 | (6S)-4-(2-chloro-9-(6- chloro-5-cyclopropyl-1- (tetrahydro-2H-pyran-2-yl)- 1H-indazol-4- yl)pyrazino[l′,2′:1,5]pyrrolo [3,2-d]pyrimidin-4-yl)-6- methyl-1,4-oxazepan-6-ol | 608 |

### Intermediate 170: (6S)-4-(2,10-dichloro-9-(5-cyclopropyl-6-methyl-1-(tetrahydro-2H-pyran-2-yl)-1H-indazol-4-yl)pyrazino[1′,2′:1,5]pyrrolo[3,2-d]pyrimidin-4-yl)-6-methyl-1,4-oxazepan-6-ol (Int-170)

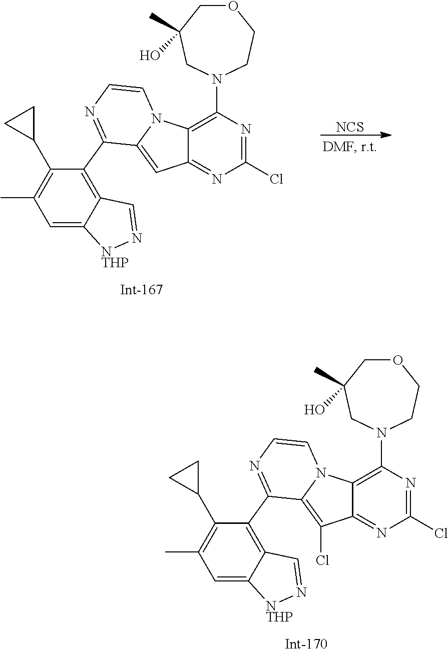

To a stirred solution of (6S)-4-(2-chloro-9-(5-cyclopropyl-6-methyl-1-(tetrahydro-2H-pyran-2-yl)-1H-indazol-4-yl)pyrazino[11,2′:1,5]pyrrolo[3,2-d]pyrimidin-4-yl)-6-methyl-1,4-oxazepan-6-ol (Int-167) (92.9 mg, 0.158 mmol) in DMF (0.527 mL) was added NCS (31.6 mg, 0.237 mmol) and the reaction was stirred at room temperature. After 24 h, the reaction was diluted with water, and extracted with EtOAc (3×). The combined organic layers were washed with brine, dried over Na₂SO₄, filtered, and concentrated. The residue was purified via silica gel chromatography (0 to 40% MeOH in EtOAc) to give (6S)-4-(2,10-dichloro-9-(5-cyclopropyl-6-methyl-1-(tetrahydro-2H-pyran-2-yl)-1H-indazol-4-yl)pyrazino[1′,2′:1,5]pyrrolo[3,2-d]pyrimidin-4-yl)-6-methyl-1,4-oxazepan-6-ol (Int-170). MS (ESI): m/z (M+H)⁺ 622.

The compounds in the table below were synthesized using a similar procedure as described in the synthesis of Int-170 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | | [M + H]⁺ |
|---|---|---|---|---|
| Int. | Starting Material | Structure | Compound Name | Found |
| Int- 171 | 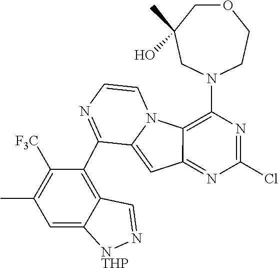 | 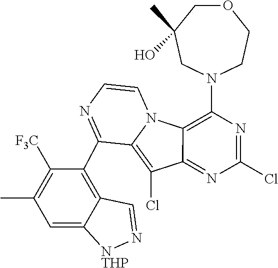 | (6S)-4-(2,10- dichloro-9-(6- methyl-1- (tetrahydro-2H- pyran-2-yl)-5- (trifluoromethyl)- 1H-indazol-4- yl)pyrazino [1′,2′:1,5]pyrrolo [3,2-d]pyrimidin- 4-yl)-6-methyl- 1,4-oxazepan-6-ol | 650 |
| | Int-168 | | | |
| Int- 172 | 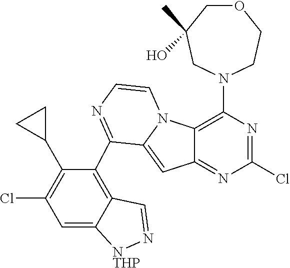 | 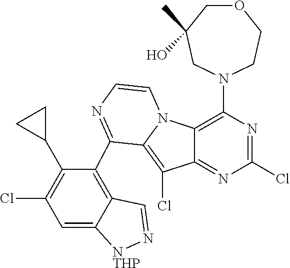 | (6S)-4-(2,10- dichloro-9-(6- chloro-5- cyclopropyl-1- (tetrahydro- 2H-pyran-2-yl)- 1H-indazol-4- yl)pyrazino [1′,2′:1,5]pyrrolo [3,2-d]pyrimidin- 4-yl)-6-methyl- 1,4-oxazepan-6-ol | 642 |
| | Int-169 | | | |

### Intermediate 173: (6S)-4-(2-chloro-9-(5-cyclopropyl-6-methyl-1-(tetrahydro-2H-pyran-2-yl)-1H-indazol-4-yl)-10-methylpyrazino[1′2′:1,5]pyrrolo[3,2-d]pyrimidin-4-yl)-6-methyl-1,4-oxazepan-6-ol (Int-173)

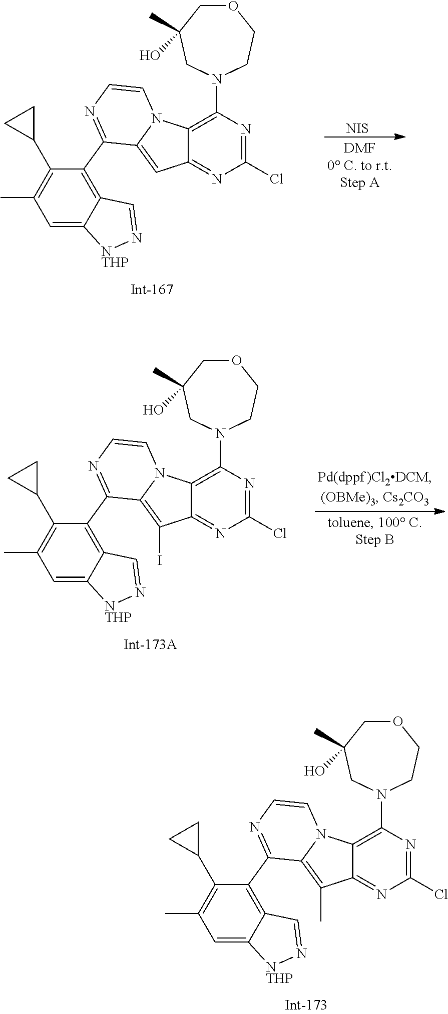

### Step A: (6S)-4-(2-chloro-9-(5-cyclopropyl-6-methyl-1-(tetrahydro-2H-pyran-2-yl)-1H-indazol-4-yl)-10-iodopyrazino[1′,2′:1,5]pyrrolo[3,2-d]pyrimidin-4-yl)-6-methyl-1,4-oxazepan-6-ol (Int-173A)

To a stirred solution of (6S)-4-(2-chloro-9-(5-cyclopropyl-6-methyl-1-(tetrahydro-2H-pyran-2-yl)-1H-indazol-4-yl)pyrazino[1′,2′:1,5]pyrrolo[3,2-d]pyrimidin-4-yl)-6-methyl-1,4-oxazepan-6-ol (Int-167) (10 mg, 0.017 mmol) in DMF (0.34 mL) was added NIS (4.2 mg, 0.019 mmol) at 0° C. and the reaction was stirred at 0° C. for 1 h and then room temperature for 3 h. The reaction was diluted with water and extracted with EtOAc (3×). The combined organic layers were washed with brine, dried over Na₂SO₄, filtered, and concentrated. The residue was purified via silica gel chromatography (0 to 15% MeOH in CHCl₃) to provide (6S)-4-(2-chloro-9-(5-cyclopropyl-6-methyl-1-(tetrahydro-2H-pyran-2-yl)-1H-indazol-4-yl)-10-iodopyrazino[1′,2′:1,5]pyrrolo[3,2-d]pyrimidin-4-yl)-6-methyl-1,4-oxazepan-6-ol (Int-173A). MS (ESI): m/z (M+H)⁺ 714. ¹H NMR (400 MHz, CDCl₃) δ 8.18-8.17 (m, 1H), 8.01-8.00 (m, 1H), 7.66-7.39 (m, 2H), 6.13-5.91 (m, 1H), 5.80-5.70 (m, 1H), 4.13-4.00 (m, 3H), 3.87-3.67 (m, 5H), 3.43-3.36 (m, 2H), 2.65-2.63 (m, 3H), 2.54-2.50 (m, 1H), 2.14-2.11 (m, 2H), 2.06-1.99 (m, 4H), 1.39-1.38 (m, 3H), 0.95-0.84 (m, 1H), 0.53-0.49 (m, 1H), 0.31-0.14 (m, 1H), 0.06-0.04 (m, 0.5H), −0.11-0.13 (m, 0.5H).

### Step B: (6S)-4-(2-chloro-9-(5-cyclopropyl-6-methyl-1-(tetrahydro-2H-pyran-2-yl)-1H-indazol-4-yl)-10-methylpyrazino[1′,2′:1,5]pyrrolo[3,2-d]pyrimidin-4-yl)-6-methyl-1,4-oxazepan-6-ol (Int-173)

A round bottom flask with a stir bar was charged with (6S)-4-(2-chloro-9-(5-cyclopropyl-6-methyl-1-(tetrahydro-2H-pyran-2-yl)-1H-indazol-4-yl)-10-iodopyrazino[1′,2′:1,5]pyrrolo[3,2-d]pyrimidin-4-yl)-6-methyl-1,4-oxazepan-6-ol (Int-173A) (13.2 mg, 0.0185 mmol), Pd(dppf)Cl₂ dichloromethane complex (2.7 mg, 0.0037 mmol), Cs₂CO₃ (30.1 mg, 0.092 mmol), trimethylboroxine (2.6 μL, 0.0185 mmol) and toluene (0.40 mL). The mixture was degassed with nitrogen (3×) and the reaction mixture was stirred at 100° C. for 2.5 h. After cooling, the mixture was diluted with EtOAc and water. The mixture was extracted with EtOAc (3×) and the combined organic layers were washed with brine, dried over Na₂SO₄, filtered, and concentrated. The residue was purified via silica gel chromatography (0 to 20% MeOH in CHCl₃) to provide (6S)-4-(2-chloro-9-(5-cyclopropyl-6-methyl-1-(tetrahydro-2H-pyran-2-yl)-1H-indazol-4-yl)-10-methylpyrazino[11,2′:1,5]pyrrolo[3,2-d]pyrimidin-4-yl)-6-methyl-1,4-oxazepan-6-ol (Int-173). MS (ESI): m/z (M+H)⁺ 602.

The compounds in the table below were synthesized using a similar procedure as described in the synthesis of Int-173 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | | [M + H]⁺ |
|---|---|---|---|---|
| Int. | Starting Material | Structure | Compound Name | Found |
| Int- 174 | 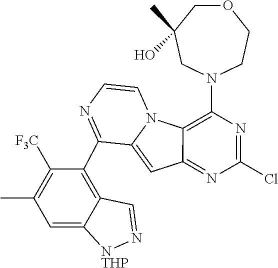 | 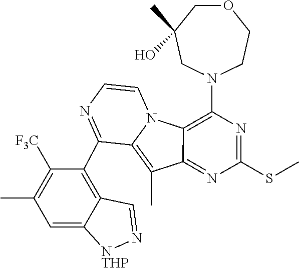 | (6S)-6-methyl-4-(10- methyl-9-(6-methyl- 1-(tetrahydro- 2H-pyran-2- yl)-5-(trifluoro- methyl)-1H- indazol-4-yl)-2- (methylthio)pyrazino [l′,2′:1,5]pyrrolo[3,2- d]pyrimidin-4-yl)- 1,4-oxazepan-6-ol | 642 |
| | Int-168 | | | |
| Int- 175 | 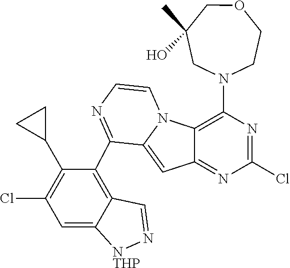 | 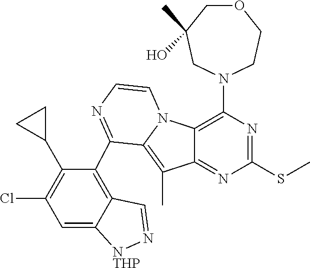 | (6S)-4-(9- (6-chloro-5- cyclopropyl-1- (tetrahydro- 2H-pyran-2-yl)-1H- indazol-4-yl)- 10-methyl-2- (methylthio)pyrazino [1′,2′:1,5]pyrrolo[3,2- d]pyrimidin-4-yl)-6- methyl-1,4- oxazepan-6-ol | 634 |
| | Int-169 | | | |

### Intermediate 176: (S)-4-(9-chloro-10-fluoro-2-(methylthio)pyrazino[1′,2′:1,5]pyrrolo[3,2-d]pyrimidin-4-yl)-6-methyl-1,4-oxazepan-6-ol (Int-176)

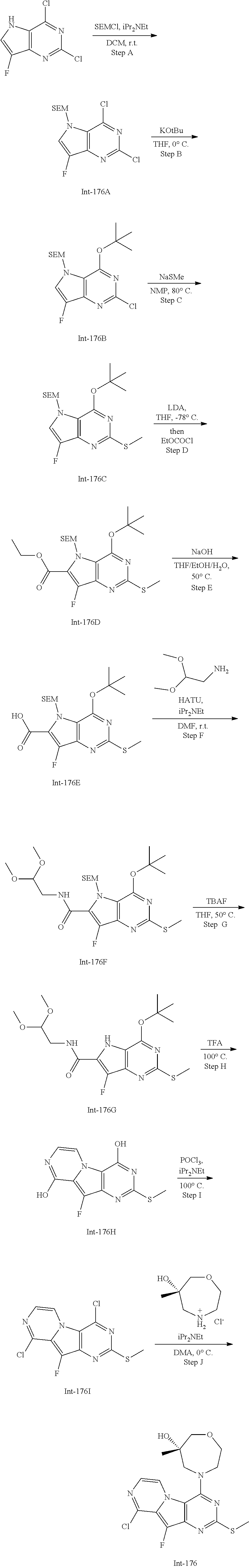

### Step A: 2,4-dichloro-7-fluoro-5-((2-(trimethylsilyl)ethoxy)methyl)-5H-pyrrolo[3,2-d]pyrimidine (Int-176A)

SEMCl (8.81 mL, 49.8 mmol) and N,N-diisopropylethylamine (14.3 mL, 83.0 mmol) were added to a solution of 2,4-dichloro-7-fluoro-5H-pyrrolo[3,2-d]pyrimidine (8.55 g, 41.5 mmol) in DCM (240 mL). After stirring at room temperature for 1 h, the mixture was diluted with EtOAc, washed with sat aq. NaHCO₃ and brine, then dried over Na₂SO₄, filtered, and evaporated. The residue was purified by silica gel chromatography (hexane/EtOAc) to afford 2,4-dichloro-7-fluoro-5-((2-(trimethylsilyl)ethoxy)methyl)-5H-pyrrolo[3,2-d]pyrimidine (Int-176A). MS (ESI): m/z (M+H)⁺ 336, 338, 340.

### Step B: 4-(tert-butoxy)-2-chloro-7-fluoro-5-((2-(trimethylsilyl)ethoxy)methyl)-5H-pyrrolo[3,2-d]pyrimidine (Int-176B)

KOtBu (32 mL, 32 mmol, 1 M in THF) was added to a solution of 2,4-dichloro-7-fluoro-5-((2-(trimethylsilyl)ethoxy)methyl)-5H-pyrrolo[3,2-d]pyrimidine (Int-176A) (8.9 g, 26 mmol) in THF (60 mL) at 0° C. After stirring at 0° C. for 1 h, the mixture was diluted with EtOAc and washed with sat. aq. NH₄Cl and brine. The organic phase was dried over Na₂SO₄, filtered, and concentrated under reduced pressure to obtain 4-(tert-butoxy)-2-chloro-7-fluoro-5-((2-(trimethylsilyl)ethoxy)methyl)-5H-pyrrolo[3,2-d]pyrimidine (Int-176B), which was used in the next step without further purification. MS (ESI): m/z (M+H)⁺ 374, 376.

### Step C: 4-(tert-butoxy)-7-fluoro-2-(methylthio)-5-((2-(trimethylsilyl)ethoxy)methyl)-5H-pyrrolo[3,2-d]pyrimidine (Int-176C)

NaSMe (3.75 g, 57.5 mmol) was added to a solution of 4-(tert-butoxy)-2-chloro-7-fluoro-5-((2-(trimethylsilyl)ethoxy)methyl)-5H-pyrrolo[3,2-d]pyrimidine (Int-176B) (10.0 g, 26.7 mmol) in NMP (40 mL) and the reaction was heated to 80° C. After stirring at 80° C. for 1 h, the mixture was diluted with EtOAc, washed with brine, then dried over Na₂SO₄, filtered, and evaporated. The residue was purified by silica gel chromatography (hexane/EtOAc) to afford 4-(tert-butoxy)-7-fluoro-2-(methylthio)-5-((2-(trimethylsilyl)ethoxy)methyl)-5H-pyrrolo[3,2-d]pyrimidine (Int-176C). MS (ESI): m/z (M−/Bu+H)⁺ 330. ¹H NMR (400 MHz, CDCl₃) δ 7.13 (d, J=2.8 Hz, 1H), 5.55 (d, J=0.8 Hz, 2H), 3.49-3.45 (m, 2H), 2.61 (s, 3H), 1.72 (s, 9H), 0.89-0.85 (m, 2H), −0.03-−0.05 (m, 9H).

### Step D: Ethyl 4-(tert-butoxy)-7-fluoro-2-(methylthio)-5-((2-(trimethylsilyl)ethoxy)methyl)-5H-pyrrolo[3,2-d]pyrimidine-6-carboxylate (Int-176D)

LDA (35 mL, 70 mmol, 2 M in THF/hexanes) was added to a solution of 4-(tert-butoxy)-7-fluoro-2-(methylthio)-5-((2-(trimethylsilyl)ethoxy)methyl)-5H-pyrrolo[3,2-d]pyrimidine (Int-176C) (9.0 g, 23 mmol) in THF (100 mL) at −78° C. After stirring at −78° C. for 1 h, a solution of ethyl chloroformate (11 mL, 120 mmol) in THF (30 mL) was added to the mixture. After stirring at −78° C. for 1 h, the mixture was diluted with EtOAc, washed with sat. aq. NH₄Cl and brine, then dried over Na₂SO₄, filtered, and evaporated. The residue was purified by silica gel chromatography (hexane/EtOAc) to afford ethyl 4-(tert-butoxy)-7-fluoro-2-(methylthio)-5-((2-(trimethylsilyl)ethoxy)methyl)-5H-pyrrolo[3,2-d]pyrimidine-6-carboxylate (Int-176D). MS (ESI): m/z (M+H)⁺ 458.

### Step E: 4-(tert-butoxy)-7-fluoro-2-(methylthio)-5-((2-(trimethylsilyl) ethoxy)methyl)-5H-pyrrolo[3,2-d]pyrimidine-6-carboxylic acid (Int-176E)

NaOH (13 mL, 66 mmol, 5 N in H₂O) was added to a solution of ethyl 4-(tert-butoxy)-7-fluoro-2-(methylthio)-5-((2-(trimethylsilyl)ethoxy)methyl)-5H-pyrrolo[3,2-d]pyrimidine-6-carboxylate (Int-176D) (10 g, 22 mmol) in THF (50 mL) and EtOH (50 mL). The reaction was warmed to 50° C. and stirred for 1 h. After 1 h, the mixture was quenched with HCl (13 mL, 5 N in H₂O) and extracted with EtOAc (3×100 mL). The combined organic layers were washed with brine, dried over sodium sulfate, filtered, and concentrated under reduced pressure to obtain 4-(tert-butoxy)-7-fluoro-2-(methylthio)-5-((2-(trimethylsilyl)ethoxy)methyl)-5H-pyrrolo[3,2-d]pyrimidine-6-carboxylic acid (Int-176E), which was used in the next step without further purification. MS (ESI): m/z (M+H)⁺ 430.

### Step F: 4-(tert-butoxy)-N-(2,2-dimethoxyethyl)-7-fluoro-2-(methylthio)-5-((2-(trimethylsilyl)ethoxy)methyl)-5H-pyrrolo[3,2-d]pyrimidine-6-carboxamide (Int-176F)

Aminoacetaldehyde dimethyl acetal (2.3 g, 22 mmol), HATU (13 g, 33 mmol), N,N-diisopropylethylamine (11 mL, 66 mmol) were added to a solution of 4-(tert-butoxy)-7-fluoro-2-(methylthio)-5-((2-(trimethylsilyl)ethoxy)methyl)-5H-pyrrolo[3,2-d]pyrimidine-6-carboxylic acid (Int-176E) (9.5 g, 22.1 mmol) in DMF (50 mL). After stirring at room temperature for 30 min, the mixture was diluted with EtOAc, washed with H₂O and brine, then dried over Na₂SO₄, filtered, and evaporated. The residue was purified by silica gel chromatography (hexane/EtOAc) to afford 4-(tert-butoxy)-N-(2,2-dimethoxyethyl)-7-fluoro-2-(methylthio)-5-((2-(trimethylsilyl)ethoxy)methyl)-5H-pyrrolo[3,2-d]pyrimidine-6-carboxamide (Int-176F). MS (ESI): m/z (M+H)⁺ 517.

### Step G: 4-(tert-butoxy)-N-(2,2-dimethoxyethyl)-7-fluoro-2-(methylthio)-5H-pyrrolo[3,2-d]pyrimidine-6-carboxamide (Int-176G)

TBAF (15 mL, 15 mmol, 1M in THF) was added to a solution of 4-(tert-butoxy)-N-(2,2-dimethoxyethyl)-7-fluoro-2-(methylthio)-5-((2-(trimethylsilyl)ethoxy)methyl)-5H-pyrrolo[3,2-d]pyrimidine-6-carboxamide (Int-176F) (1.6 g, 3.1 mmol) in THF (3 mL) at room temperature and warmed to 50° C. After stirring at 50° C. for 24 h, the mixture was diluted with EtOAc, washed with 1 N HCl and brine, dried over Na₂SO₄, filtered, and evaporated to obtain 4-(tert-butoxy)-N-(2,2-dimethoxyethyl)-7-fluoro-2-(methylthio)-5H-pyrrolo[3,2-d]pyrimidine-6-carboxamide (Int-176G), which was used in the next step without further purification. MS (ESI): m/z (M+H)⁺ 387.

### Step H: 10-fluoro-2-(methylthio)pyrazino[1′,2′:1,5]pyrrolo[3,2-d]pyrimidine-4,9-diol (Int-176H)

TFA (9.7 mL, 130 mmol) was added to 4-(tert-butoxy)-N-(2,2-dimethoxyethyl)-7-fluoro-2-(methylthio)-5H-pyrrolo[3,2-d]pyrimidine-6-carboxamide (Int-176G) (1.1 g, 2.8 mmol) and the reaction was warmed to 100° C. After stirring at 100° C. for 30 min, the reaction was cooled to room temperature and MeOH (30 mL) was added to the reaction mixture. The precipitated solid was filtered off and dried to give 10-fluoro-2-(methylthio)pyrazino[1,2′:1,5]pyrrolo[3,2-d]pyrimidine-4,9-diol (Int-176H). MS (ESI): m/z (M+H)⁺ 267. ¹H NMR (400 MHz, DMSO-d₆) δ 11.08 (d, J=5.3 Hz, 1H), 7.85 (d, J=6.0 Hz, 1H), 6.75 (t, J=5.6 Hz, 1H), 2.56 (s, 3H).

### Step 1:4,9-dichloro-10-fluoro-2-(methylthio)pyrazino[1′,2′:1,5]pyrrolo[3,2-d]pyrimidine (Int-1761)

Phosphoryl chloride (4.85 mL, 52.1 mmol) and N,N-diisopropylethylamine (2.50 mL, 14.6 mmol) were added to 10-fluoro-2-(methylthio)pyrazino[1′,2′:1,5]pyrrolo[3,2-d]pyrimidine-4,9-diol (Int-176H) (775 mg, 2.91 mmol) and the reaction was warmed to 100° C. After stirring at 100° C. for 24 h, the reaction mixture was concentrated. The mixture was diluted with EtOAc, washed with brine, dried over Na₂SO₄, filtered, and evaporated to obtain 4,9-dichloro-10-fluoro-2-(methylthio)pyrazino[1′,2′:1,5]pyrrolo[3,2-d]pyrimidine (Int-1761), which was used in the next step without further purification. MS (ESI): m/z (M+H)⁺ 303, 305.

### Step J: (S)-4-(9-chloro-10-fluoro-2-(methylthio)pyrazino[1′,2′:1,5]pyrrolo[3,2-d]pyrimidin-4-yl)-6-methyl-1,4-oxazepan-6-ol (Int-176)

(S)-6-methyl-1,4-oxazepan-6-ol hydrochloride (1.22 mL, 7.28 mmol) and N,N-diisopropylethylamine (5.00 mL, 29.2 mmol) were added to crude 4,9-dichloro-10-fluoro-2-(methylthio)pyrazino[1′,2′:1,5]pyrrolo[3,2-d]pyrimidine (Int-1761) in DMA (9.70 mL) at 0° C. After stirring at 0° C. for 2 h, the mixture was diluted with EtOAc, washed with sat. aq. NH₄Cl and brine, dried over Na₂SO₄, filtered, and evaporated to obtain(S)-4-(9-chloro-10-fluoro-2-(methylthio)pyrazino[1′,2′:1,5]pyrrolo[3,2-d]pyrimidin-4-yl)-6-methyl-1,4-oxazepan-6-ol (Int-176). MS (ESI): m/z (M+H)⁺ 398, 400.

### Intermediate 177:4-(benzyloxy)-6-chloro-5-(2-chloro-3-fluoropyridin-4-yl)-2-(methylthio)pyrimidine (Int-177)

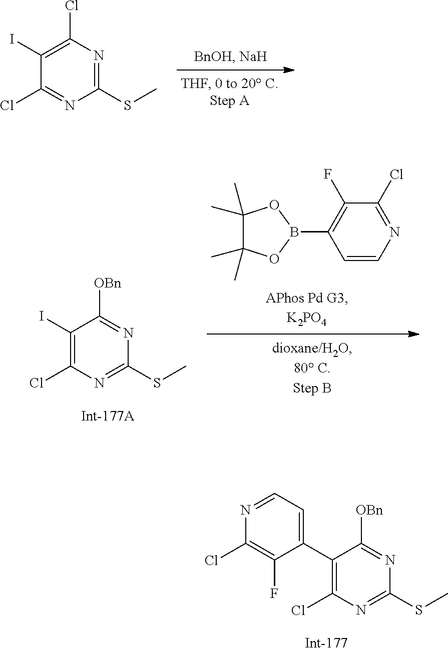 [SMILES: CSc1nc(Cl)c(I)c(Cl)n1 ; CC1(C)OB(c2ccnc(Cl)c2F)OC1(C)C ; CSc1nc(Cl)c(I)c(OCc2ccccc2)n1 ; CSc1nc(Cl)c(-c2ccnc(Cl)c2F)c(OCc2ccccc2)n1]

### Step A: 4-(benzyloxy)-6-chloro-5-iodo-2-(methylthio)pyrimidine (Int-177A)

NaH (149.5 g, 3.739 mol, 60 wt % in mineral oil) was added to a stirred solution of benzyl alcohol (404.3 g, 3.738 mol) in THF (5 L) in portions at 0° C. under nitrogen atmosphere. The resulting mixture was stirred for 1 h at 0° C. under nitrogen atmosphere. The reaction mixture was added dropwise to 4,6-dichloro-5-iodo-2-(methylsulfanyl)pyrimidine (1000 g, 3.115 mol) in THF (10 L) over 1 h at 20° C. Then resulting mixture was stirred for additional 1 h 20° C. The reaction was poured into the ice water (10 L) and extracted with EtOAc (2×10 L). The combined organic layers were washed with brine (1×10 L), dried over anhydrous Na₂SO₄, filtered, and the filtrate was concentrated under reduced pressure. The residue was purified by trituration with MeOH (5.0 L) to afford 4-(benzyloxy)-6-chloro-5-iodo-2-(methylthio)pyrimidine (Int-177A). MS (ESI): m/z (M+H)⁺ 393.

### Step B: 4-(benzyloxy)-6-chloro-5-(2-chloro-3-fluoropyridin-4-yl)-2-(methylthio)pyrimidine (Int-177)

K₃PO₄ (946.1 g, 4.457 mol) and APhos Pd G3 (56.61 g, 89.14 mmol) were added to a solution of 4-(benzyloxy)-6-chloro-5-iodo-2-(methylthio)pyrimidine (Int-177A) (700 g, 1.78 mol) in dioxane (8 L) and H₂O (1 L). A solution of 2-chloro-3-fluoro-4-(4,4,5,5-tetramethyl-1,3,2-dioxaborolan-2-yl)pyridine (918.1 g, 3.565 mmol) in dioxane (2 L) was added dropwise over 2 h at 80° C. under nitrogen atmosphere. The reaction was quenched by the addition of water (10 L). The resulting mixture was extracted with EtOAc (2×10 L). The combined organic layers were washed with brine (1×10 L), dried over anhydrous Na₂SO₄, filtered, and the filtrate was concentrated under reduced pressure. The residue was purified by silica gel column chromatography (20:1 petroleum ether:EtOAc) to afford 4-(benzyloxy)-6-chloro-5-(2-chloro-3-fluoropyridin-4-yl)-2-(methylthio)pyrimidine (Int-177). MS (ESI): m/z (M+H)⁺ 396.

### Intermediate 178: Tert-butyl 1-fluoro-3,8-diazabicyclo[3.2.1]octane-8-carboxylate (Int-178)

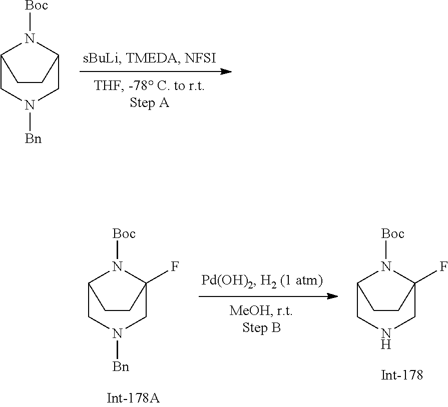 [SMILES: CC(C)(C)OC(=O)N1C2CCC1CN(Cc1ccccc1)C2 ; CC(C)(C)OC(=O)N1C2CCC1(F)CN(Cc1ccccc1)C2 ; CC(C)(C)OC(=O)N1C2CCC1(F)CNC2]

### Step A: Tert-butyl 3-benzyl-1-fluoro-3,8-diazabicyclo[3.2.1]octane-8-carboxylate (Int-178A)

Tert-butyl(1R,5S)-3-benzyl-3,8-diazabicyclo[3.2.1]octane-8-carboxylate (1.00 g, 3.31 mmol) was added to a round bottom flask with a stir bar. The flask was evacuated and back filled with nitrogen (3×). Dry THF (20.0 mL) and TMEDA (0.56 mL, 3.70 mmol) was added via a syringe. The reaction was cooled to −78° C. and sec-butyllithium (3.54 mL, 4.96 mmol) was added dropwise via syringe, keeping the internal temperature below −60° C. The solution was stirred at −78° C. for 30 mins. To this solution was added N-fluorobenzenesulfonamide (2.09 g, 6.61 mmol) as a solution in THF (20.00 mL). The mixture was stirred for 30 min at −78° C. and allowed to slowly warm to r.t. Water (20 mL) was added to the solution and the organic layer was extracted with EtOAc, dried over MgSO₄, then filtered and concentrated. The crude product was purified via silica gel chromatography (0 to 100% EtOAc in hexanes) to provide tert-butyl 3-benzyl-1-fluoro-3,8-diazabicyclo[3.2.1]octane-8-carboxylate (Int-178A), MS (ESI): m/z (M+H)⁺ 321.

### Step B: Tert-butyl 1-fluoro-3,8-diazabicyclo[3.2.1]octane-8-carboxylate (Int-178)

Tert-butyl 3-benzyl-1-fluoro-3,8-diazabicyclo[3.2.1]octane-8-carboxylate (Int-178A) (100 mg, 0.312 mmol) was dissolved in MeOH (1.56 mL). The reaction was purged with nitrogen at 1 atm and palladium hydroxide on carbon (22 mg, 0.03 mmol) was added. The reaction was flushed with nitrogen, then the atmosphere was replaced with hydrogen. The reaction mixture was stirred at r.t. overnight. The reaction was filtered through a pad of CELITE® and concentrated in vacuo to provide tert-butyl 1-fluoro-3,8-diazabicyclo[3.2.1]octane-8-carboxylate (Int-178), which was used directly in the next step without further purification. MS (ESI): m/z (M-t-Bu)+175.

### Intermediate 179: 6-oxa-1,9-diazaspiro[3.6]decan-2-one (Int-179)

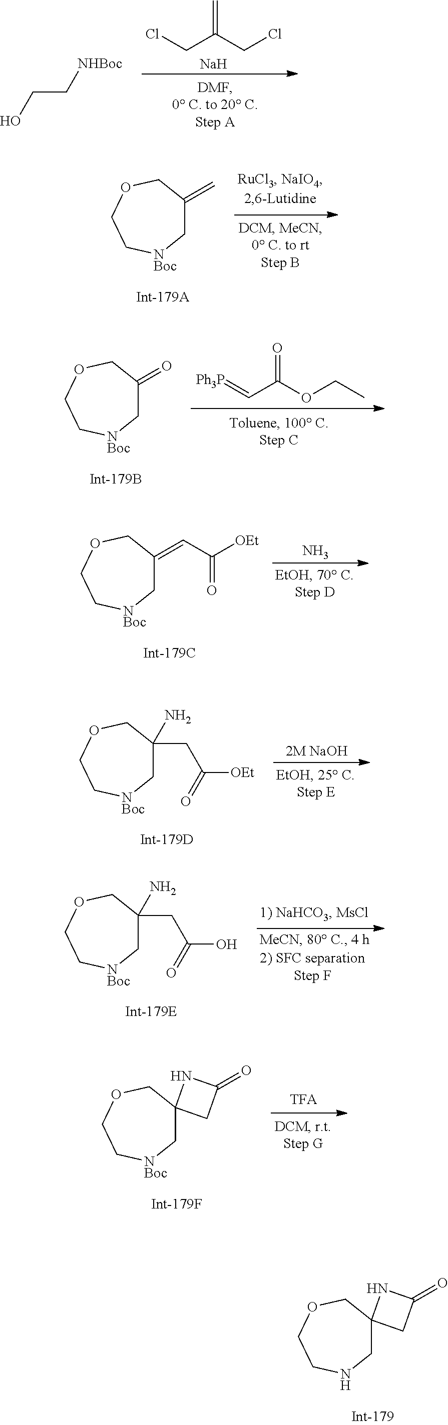

### Step A: Tert-butyl 6-methylene-1,4-oxazepane-4-carboxylate (Int-179A)

To a stirred solution of tert-butyl(2-hydroxyethyl) carbamate (15 g, 93 mmol) and 3-chloro-2-(chloromethyl) prop-1-ene (12 g, 97 mmol) in DMF (100 mL) under argon was added NaH (8.2 g, 205 mmol, 60 wt % in mineral oil) at 0° C. The reaction mixture was stirred at 20° C. for 12 h. The reaction mixture was quenched with ice cold water (200 mL) and extracted with ether (2×200 mL). The combined organic layers were dried over sodium sulfate and concentrated under reduced pressure. The crude compound was purified by silica gel chromatography over (10% EtOAc in petroleum ether) to obtain tert-butyl 6-methylene-1,4-oxazepane-4-carboxylate (Int-179A). ¹H NMR (400 MHz, CDCl₃) δ 4.90-5.08 (m, 2H), 4.0-4.20 (m, 4H), 3.65-3.75 (m, 2H), 3.44-3.60 (m, 2H), 1.46 (s, 9H).

### Step B: Tert-butyl 6-oxo-1,4-oxazepane-4-carboxylate (Int-179B)

To a stirred solution of tert-butyl 6-methylene-1,4-oxazepane-4-carboxylate (Int-179A) (15.0 g, 7.03 mmol) in DCM (250 mL) and MeCN (250 mL) was added 2,6-lutidine (16.4 mL, 141 mmol), water (750 mL), and sodium periodate (60.0 g, 281 mmol) at 0° C. Then to the reaction mixture was added a stock solution of ruthenium (III) chloride (20.1 mL, 0.703 mmol) in water dropwise at r.t. The reaction mixture was stirred at 25° C. for 6 h. The reaction mixture was quenched with water (200 mL) and extracted with DCM (2×100 mL). The combined organic layers were dried over sodium sulfate and concentrated under reduced pressure. The crude compound was purified by silica gel chromatography (10% EtOAc in petroleum ether) to obtain tert-butyl 6-oxo-1,4-oxazepane-4-carboxylate (Int-179B). ¹H NMR (400 MHz, CDCl₃) δ 3.98-4.18 (m, 4H), 3.90 (s, 2H), 3.69 (s, 2H), 1.45 (s, 9H).

### Step C: Tert-butyl(E)-6-(2-ethoxy-2-oxoethylidene)-1,4-oxazepane-4-carboxylate (Int-179C)

To a stirred solution of tert-butyl 6-oxo-1,4-oxazepane-4-carboxylate (Int-179B) (19 g, 88 mmol) in toluene (500 mL) was added (carbethoxymethylene)triphenylphosphorane (77 g, 221 mmol) at r.t. The reaction mixture was stirred at 100° C. for 18 h. The reaction mixture was concentrated under reduced pressure. The crude compound was purified by silica gel chromatography (4% EtOAc in petroleum ether) to obtain tert-butyl(E)-6-(2-ethoxy-2-oxoethylidene)-1,4-oxazepane-4-carboxylate (Int-179C). MS (ESI): m/z (M+H)⁺ 286.

### Step D: tert-butyl 6-amino-6-(2-ethoxy-2-oxoethyl)-1,4-oxazepane-4-carboxylate (Int-179D)

To a stirred solution of tert-butyl(E)-6-(2-ethoxy-2-oxoethylidene)-1,4-oxazepane-4-carboxylate (Int-179C) (14.0 g, 46.1 mmol) in EtOH (115 mL, 231 mmol) was added a solution of ammonia in EtOH (100 mL). The reaction mixture was stirred at 70° C. for 12 h in a steel bomb. The reaction mixture was concentrated under reduced pressure. The crude compound was purified by silica gel chromatography (4% MeOH in DCM) to obtain tert-butyl 6-amino-6-(2-ethoxy-2-oxoethyl)-1,4-oxazepane-4-carboxylate (Int-179D). MS (ESI): m/z (M+H)⁺ 303.

### Step E: 2-(6-amino-4-(tert-butoxycarbonyl)-1,4-oxazepan-6-yl) acetic acid (Int-179E)

To a stirred solution of tert-butyl 6-amino-6-(2-ethoxy-2-oxoethyl)-1,4-oxazepane-4-carboxylate (Int-179D) (8.00 g, 26.5 mmol) in EtOH (80 mL) was added 2 M aq. NaOH (19.84 mL, 39.7 mmol) at r.t. The reaction mixture was stirred at 25° C. for 16 h. The reaction mixture was concentrated under reduced pressure and co-distilled with toluene (50 mL) to afford 2-(6-amino-4-(tert-butoxycarbonyl)-1,4-oxazepan-6-yl) acetic acid (Int-179E) which was used directly in next step without further purification. MS (ESI): m/z (M+H)⁺ 275.

### Step F: Tert-butyl 2-oxo-6-oxa-1,9-diazaspiro[3.6]decane-9-carboxylate (Int-179F)

To a stirred solution of sodium bicarbonate (11.0 g, 131 mmol) in MeCN (60 mL) was added MsCl (2.56 mL, 32.8 mmol) at r.t. and the temperature was increased to 80° C. Then 2-(6-amino-4-(tert-butoxycarbonyl)-1,4-oxazepan-6-yl) acetic acid (Int-179E) (6.00 g, 21.9 mmol) was added portion wise over a period of 1 h. The reaction mixture was stirred at 80° C. for 4 h then quenched with water (200 mL) and extracted with EtOAc (4×200 mL). The combined organic layers were dried over anhydrous sodium sulfate and concentrated under reduced pressure. The crude compound was purified by silica gel chromatography (5% MeOH in DCM) to obtain racemic tert-butyl 2-oxo-6-oxa-1,9-diazaspiro[3.6]decane-9-carboxylate. The enantiomers were separated by chiral SFC (Column J, 15% MeOH) to afford tert-butyl 2-oxo-6-oxa-1,9-diazaspiro[3.6]decane-9-carboxylate (Int-179F) (second eluting isomer from SFC). MS (ESI): m/z (M+H)⁺ 257. ¹H NMR (400 MHz, DMSO-d₆): δ (ppm) 8.20-8.34 (m, 1H), 3.51-3.80 (m, 6H), 3.45 (t, J=14.6 Hz, 1H), 3.19-3.28 (m, 1H), 2.72-2.80 (m, 1H), 2.58-2.70 (m, 1H), 1.41 (s, 9H).

### Step G: 6-oxa-1,9-diazaspiro[3.6]decan-2-one carboxylate (Int-179)

To a solution of tert-butyl 2-oxo-6-oxa-1,9-diazaspiro[3.6]decane-9-carboxylate (Int-37F) (100 mg, 0.390 mmol) in DCM (0.60 mL) was added TFA (0.20 mL, 2.60 mmol) at 25° C. The reaction mixture was stirred at 25° C. for 30 min. The reaction mixture was evaporated under reduced pressure to give 6-oxa-1,9-diazaspiro[3.6]decan-2-one (Int-179), isolated as a TFA salt. The crude product was used directly in the next step without further purification. ¹H NMR (500 MHz, DMSO-d₆) δ 8.31 (s, 1H), 3.90-3.86 (m, 1H), 3.84-3.76 (m, 2H), 3.56 (d, J=13.5 Hz, 1H), 3.39 (d. J=13.5 Hz, 1H), 3.31 (brs, 2H), 3.18 (s, 2H), 2.91 (d, J=14.7 Hz, 1H), 2.78 (d, J=14.7 Hz, 1H).

### Intermediate 180:1,7-dioxa-10-azaspiro[4.6]undecane (Int-180)

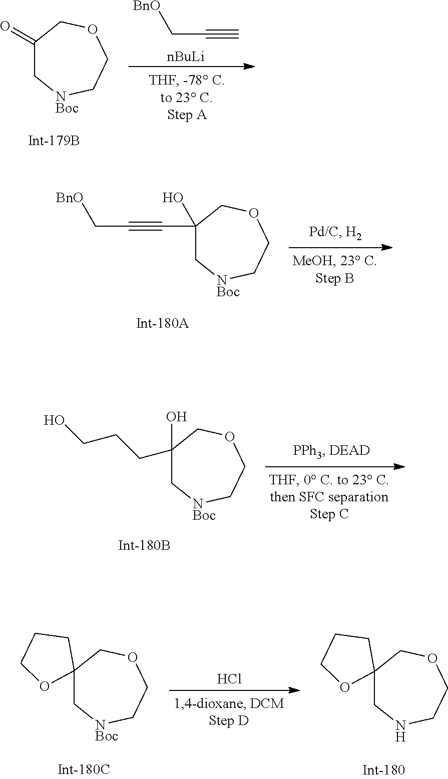

### Step A: Tert-butyl 6-(3-(benzyloxy) prop-1-yn-1-yl)-6-hydroxy-1,4-oxazepane-4-carboxylate (Int-180A)

A 100 mL round-bottom flask with a stir bar was placed under nitrogen and THF (23 mL) and ((prop-2-yn-1-yloxy)methyl)benzene (1.6 mL, 11.2 mmol) were added via syringe. The reaction was cooled to −78° C. with a dry ice/acetone bath and nBuLi (4.5 mL, 11.2 mmol, 2.5 M in hexanes) was added via syringe. The reaction was stirred at this temperature for 20 min before tert-butyl 6-oxo-1,4-oxazepane-4-carboxylate (Int-179B) (2.00 g, 9.29 mmol) was added in one portion. The reaction was warmed to room temperature and stirred for 1 h. The reaction was quenched with sat. aq. NH₄Cl (50 mL) and diluted with EtOAc (100 mL). The mixture was transferred to a separatory funnel, shaken, and separated. The aqueous phase was washed with EtOAc (2×50 mL). The combined organics were dried over MgSO₄, filtered, and concentrated. Purified via silica gel chromatography (0 to 60% EtOAc in hexanes) to give tert-butyl 6-(3-(benzyloxy) prop-1-yn-1-yl)-6-hydroxy-1,4-oxazepane-4-carboxylate (Int-180A). MS (ESI): m/z (M+H)⁺ 362.

### Step B: Tert-butyl 6-hydroxy-6-(3-hydroxypropyl)-1,4-oxazepane-4-carboxylate (Int-180B)

To a solution of tert-butyl 6-(3-(benzyloxy) prop-1-yn-1-yl)-6-hydroxy-1,4-oxazepane-4-carboxylate (Int-180A) (2.9 g, 8.0 mmol) in MeOH (40 mL) was added palladium on carbon (598 mg, 0.562 mmol). The reaction vessel was evacuated and backfilled with H₂ three times and the reaction mixture was stirred under 1 atm (balloon) of hydrogen for 22 h. Palladium on carbon (598 mg, 0.562 mmol) was added and hydrogenation was continued for 3 h. The reaction mixture was filtered through a colite pad and concentrated. Purified via silica gel chromatography (0 to 100% EtOAc in hexanes) to give tert-butyl 6-hydroxy-6-(3-hydroxypropyl)-1,4-oxazepane-4-carboxylate (Int-180B). MS (ESI): m/z (M+H)⁺ 276.

### Step C: Tert-butyl 1,7-dioxa-10-azaspiro[4.6]undecane-10-carboxylate (Int-180C)

To a solution of tert-butyl 6-hydroxy-6-(3-hydroxypropyl)-1,4-oxazepane-4-carboxylate (Int-180B) (1.58 g, 5.74 mmol) and triphenylphosphine (1.81 g, 6.89 mmol) in THF (66 mL) was added a solution of diethyl azodicarboxylate (3.10 mL, 6.89 mmol) in THF (23 mL) at 0° C. and the reaction was stirred at the same temperature for 45 min. The cooling bath was removed, and the reaction was stirred at room temperature for 55 h. The reaction was concentrated and purified via silica gel chromatography (0 to 50% EtOAc in hexanes) to give racemic tert-butyl 1,7-dioxa-10-azaspiro[4.6]undecane-10-carboxylate. The enantiomers were separated by chiral SFC (Column I, 20% MeOH) to afford tert-butyl 1,7-dioxa-10-azaspiro[4.6]undecane-10-carboxylate (Int-180C, second eluting isomer from SFC). MS (ESI): m/z (M+H)⁺ 258.

### Step D: 1,7-dioxa-10-azaspiro[4.6]undecane (Int-180)

Tert-butyl 1,7-dioxa-10-azaspiro[4.6]undecane-10-carboxylate (Int-180C) (0.494 g, 1.92 mmol) was dissolved in DCM (19 mL) and HCl (9.5 mL, 38.0 mmol, 4 M in dioxane) was added dropwise. The reaction was stirred at room temperature for 2.5 h and then evaporated under reduced pressure to give 1,7-dioxa-10-azaspiro[4.6]undecane (Int-180) isolated as the HCl salt. MS (ESI), MS (ESI): m/z (M+H)⁺ 158.

### Intermediate 181:2-methyl-1,6-dioxa-9-azaspiro[3.6]decane (Int-181)

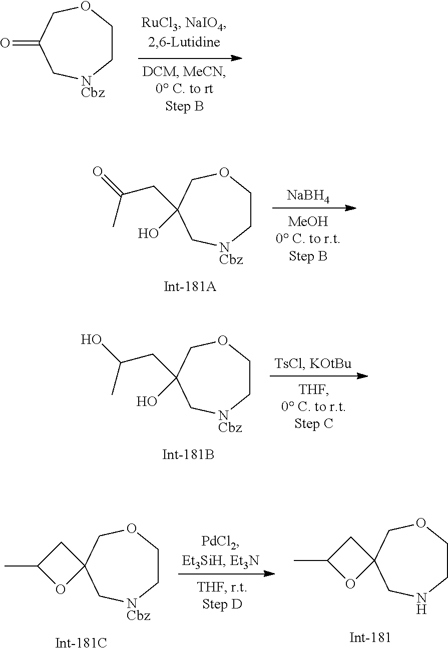

### Step A: Benzyl 6-hydroxy-6-(2-oxopropyl)-1,4-oxazepane-4-carboxylate (Int-181A)

An oven-dried vial with stir bar was placed under N₂ and THF (3.01 mL) and acetone (66.3 μL, 0.903 mmol) were added via syringe. The reaction was cooled to −78° C. Lithium diisopropylamide (451 μL, 0.903 mmol) was added dropwise and the reaction was stirred for 15 minutes. After 15 min, benzyl 6-oxo-1,4-oxazepane-4-carboxylate (75 mg, 0.301 mmol) in THF (3.01 mL) was added dropwise and the reaction was warmed to room temperature. After 1 h, the reaction was quenched with sat. aq. NH₄Cl (10 mL) and extracted with EtOAc (3×10 mL). The combined organics were washed with brine (10 mL), dried over Na₂SO₄, filtered, and concentrated to afford benzyl 6-hydroxy-6-(2-oxopropyl)-1,4-oxazepane-4-carboxylate (Int-181A), which was used directly in the next step without purification. MS (ESI): m/z (M+H)⁺ 308.

### Step B: Benzyl 6-hydroxy-6-(2-hydroxypropyl)-1,4-oxazepane-4-carboxylate (Int-181B)

A vial with stir bar was charged with benzyl 6-hydroxy-6-(2-oxopropyl)-1,4-oxazepane-4-carboxylate (Int-181A) (92 mg, 0.30 mmol) and MeOH (1.5 mL). The reaction was cooled to 0° C. and sodium borohydride (57 mg, 1.5 mmol) was added. The reaction was warmed to room temperature and stirred for 1 h. After 1 h, the reaction was quenched with sat. aq. NH₄Cl (10 mL) and extracted with EtOAc (3×10 mL). The combined organics were washed with brine (10 mL), dried over Na₂SO₄, filtered, and concentrated. The crude product was purified via silica gel chromatography (0 to 70% EtOAc in hexanes) to provide benzyl 6-hydroxy-6-(2-hydroxypropyl)-1,4-oxazepane-4-carboxylate (Int-181B). MS (ESI): m/z (M+H)⁺ 310.

### Step C: Benzyl 2-methyl-1,6-dioxa-9-azaspiro[3.6]decane-9-carboxylate (Int-181C)

An oven-dried vial with stir bar was charged with benzyl 6-hydroxy-6-(2-hydroxypropyl)-1,4-oxazepane-4-carboxylate (Int-181B) (46 mg, 0.149 mmol) and p-toluenesulfonyl chloride (28.3 mg, 0.149 mmol). The vial was fitted with a septum and placed under N₂. THF (2.97 mL) was added via syringe and the reaction was cooled to 0° C. Potassium tert-butoxide (149 μL, 0.149 mmol, 1M in THF) was added and the reaction was stirred for 30 min. Then, potassium tert-butoxide (164 μL, 0.164 mmol, 1M in THF) was added, the reaction was warmed to room temperature, and stirred for an additional 30 min. The reaction was quenched with sat. aq. NH₄Cl (10 mL) and diluted with EtOAc (10 mL). The layers were shaken and separated. The aqueous phase was extracted with EtOAc (3×10 mL) and the combined organics were dried over MgSO₄, filtered, and concentrated. The crude product was purified via silica gel chromatography (0 to 50% EtOAc in hexanes) to provide benzyl 2-methyl-1,6-dioxa-9-azaspiro[3.6]decane-9-carboxylate (Int-181C) as a clear liquid. MS (ESI): m/z (M+H)⁺ 292.

### Step D: 2-methyl-1,6-dioxa-9-azaspiro[3.6]decane (Int-181)

A vial with stir bar was charged with palladium (II) chloride (3.65 mg, 0.021 mmol), fitted with a septum, and placed under N₂. Benzyl 2-methyl-1,6-dioxa-9-azaspiro[3.6]decane-9-carboxylate (Int-180C) (12 mg, 0.041 mmol) was added as a solution in THF (824 μL) to this vial via syringe and sparged with N₂ for 15 min. Triethylamine (17.22 μL, 0.124 mmol) and triethylsilane (13.16 μL, 0.082 mmol) were added via syringe and the reaction was stirred for 1 h. After 1 h, the reaction was diluted with ether (5 mL) and filtered through celite. The reaction was concentrated in vacuo to provide 2-methyl-1,6-dioxa-9-azaspiro[3.6]decane (Int-181). MS (ESI): m/z (M+H)⁺ 158.

### Intermediate 182:6-ethyl-1,4-oxazepan-6-ol (Int-182)

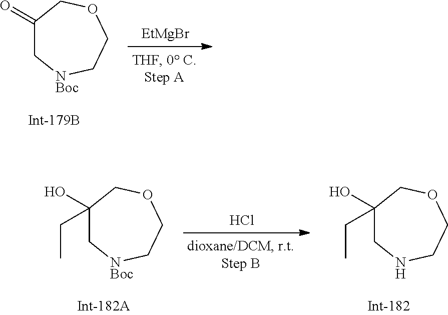

### Step A: Tert-butyl 6-ethyl-6-hydroxy-1,4-oxazepane-4-carboxylate (Int-182A)

Ethylmagnesium bromide (0.70 mL, 2.09 mmol, 3M in Et₂O) was added to a solution of tert-butyl 6-oxo-1,4-oxazepane-4-carboxylate (Int-179B) (300 mg, 1.39 mmol) in THF (4.65 mL) at 0° C. The mixture was stirred at room temperature for 14 h. The mixture was quenched with sat. aq. NH₄Cl and diluted with EtOAc. The organic layer was separated, washed with brine, and dried over Na₂SO₄. The dried solution was filtered and the filtrate was concentrated under reduced pressure. The residue obtained was purified by silica gel chromatography (hexane/EtOAc=80/20 to 50/50) to afford tert-butyl 6-ethyl-6-hydroxy-1,4-oxazepane-4-carboxylate (Int-182A). MS (ESI) [M+H]⁺: m/z 246.

### Step B: 6-ethyl-1,4-oxazepan-6-ol (Int-182)

HCl (1.70 mL, 6.81 mmol, 4 N in dioxane) was added to a solution of tert-butyl 6-ethyl-6-hydroxy-1,4-oxazepane-4-carboxylate (Int-182A) (334 mg, 1.36 mmol) in DCM (3.34 mL) at room temperature. The mixture was stirred at room temperature for 14 h and concentrated to afford 6-ethyl-1,4-oxazepan-6-ol (Int-182) as the hydrochloride salt, which was used directly in the next step without further purification. MS (ESI) [M+H]⁺: m/z 146.

### Example Syntheses

### Example 1:(6S)-4-(2-(((2R,7aS)-2-fluorotetrahydro-1H-pyrrolizin-7a(5H)-yl)methoxy)-9-methyl-8-(6-methyl-5-(trifluoromethyl)-1H-indazol-4-yl)-9H-pyrido[4′,3′:4,5]pyrrolo[2,3-d]pyrimidin-4-yl)-6-methyl-1,4-oxazepam-6-ol (Ex. 1)

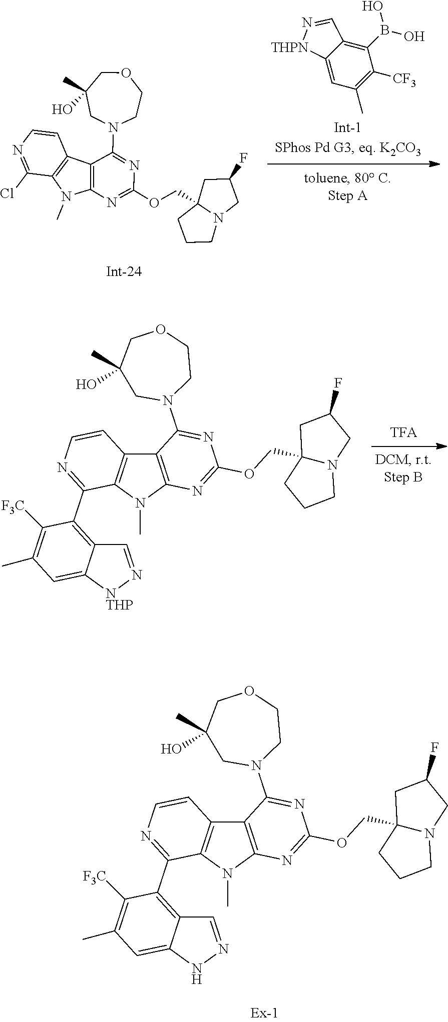

### Step A: (6S)-4-(2-(2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol

To a solution of(S)-4-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Int-137) (300 mg, 0.594 mmol) (divided into 6 batches, 50 mg each) in toluene (24 mL) was added (6-methyl-1-(tetrahydro-2H-pyran-2-yl)-5-(trifluoromethyl)-1H-indazol-4-yl)boronic acid (Int-1) (390 mg, 1.19 mmol), SPhos Pd G3 (232 mg, 0.297 mmol), and K₂CO₃ (0.891 mL, 1.78 mmol, 2 M in H₂O) at 25° C. The reaction was stirred at 80° C. for 16 h under N₂ atmosphere. LCMS showed the reaction was complete. The reaction mixture was diluted with EtOAc (30 mL), washed with brine (10 mL), and the organic layer was dried and concentrated in vacuo. The residue was purified by flash silica gel chromatography (0-50% THF in petroleum ether gradient @ 20 mL/min, dry loaded) to give rac. (6S)-4-(2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol. MS (ESI) [M+H]⁺: m/z 753.

### Step B: (6S)-4-(2-(((2R,7aS)-2-fluorotetrahydro-1H-pyrrolizin-7a(5H)-yl)methoxy)-9-methyl-8-(6-methyl-5-(trifluoromethyl)-1H-indazol-4-yl)-9H-pyrido[4′,3′:4,5]pyrrolo[2,3-d]pyrimidin-4-yl)-6-methyl-1,4-oxazepam-6-ol (Ex-1)

To a solution of (6S)-4-(2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol (160 mg, 0.213 mmol) in DCM (1 mL) was added TFA (0.5 mL), and the reaction was stirred at r.t. for 1 h. LCMS showed the reaction was complete. The reaction was concentrated in vacuo and the residue was purified to give rac (6S)-4-(2-(((2R,7aS)-2-fluorotetrahydro-1H-pyrrolizin-7a(5H)-yl)methoxy)-9-methyl-8-(6-methyl-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol (rac-Ex-1). The atropisomers were separated by preparative reverse-phase HPLC (Column A, 10 to 30% MeCN in H₂O, 0.2% formic acid modifier) to give (6S)-4-(2-(((2R,7aS)-2-fluorotetrahydro-1H-pyrrolizin-7a(5H)-yl)methoxy)-9-methyl-8-(6-methyl-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol (Ex-1) (the second eluting isomer from RP-HPLC). MS (ESI) [M+H]⁺: m/z 669. ¹H NMR (500 MHz, MeOD) δ 8.46 (d, J=5.65 Hz, 1H), 7.94 (d. J=5.65 Hz, 1H), 7.76 (s, 1H), 7.37 (s, 1H), 5.44-5.60 (m, 1H), 4.58 (br d, J=11.60 Hz, 1H), 4.41-4.50 (m, 2H), 4.20 (d, J=14.80 Hz, 1H), 3.99-4.07 (m, 2H), 3.90 (d, J=14.80 Hz, 1H), 3.63-3.77 (m, 5H), 3.47 (d, J=12.51 Hz, 1H), 3.32-3.37 (m, 1H), 3.17 (s, 3H), 2.75 (d, J=1.53 Hz, 3H), 2.44-2.63 (m, 2H), 2.30 (br s, 1H), 2.24 (br dd, J=6.18, 11.22 Hz, 2H), 2.04-2.13 (m, 1H), 1.29 (s, 3H).

The examples in the table below were synthesized using a similar procedure as described in the synthesis of Ex-1 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | SFC | | |
|---|---|---|---|---|---|
| | Starting | | Separation | Compound | [M + H]⁺ |
| Ex. | Material | Structure | Conditions | Name | Found |
| Ex- 2 | 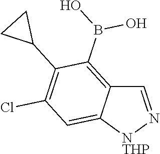 Int-33 | 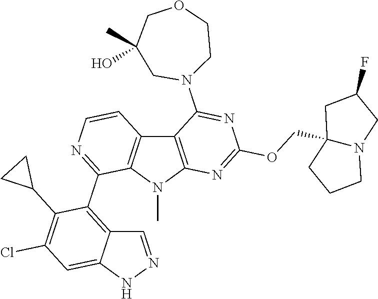 [SMILES: Cn1c2nc(OC[C@@]34CCCN3C[C@H](F)C4)nc(N3CCOC[C@@](C)(O)C3)c2c2ccnc(-c3c(C4CC4)c(Cl)cc4[nH]ncc34)c21] | Column E, 55% EtOH w/ 0.1% NH₄OH, Second eluting isomer | (6S)-4-(8-(6-chloro- 5-cyclopropyl-1H- indazol-4-yl)-2- (((2R,7aS)-2- fluorotetrahydro- 1H-pyrrolizin- 7a(5H)- yl)methoxy)-9- methyl-9H-pyrido [4′,3′:4,5]pyrrolo [2,3-d]pyrimidin- 4-yl)-6-methyl-1,4- oxazepan-6-ol | 661 |
| Ex- 3 | 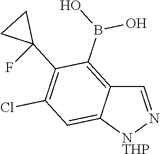 Int-44 | 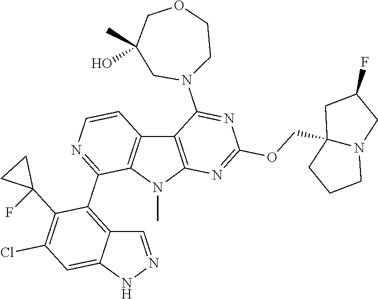 [SMILES: Cn1c2nc(OC[C@@]34CCCN3C[C@H](F)C4)nc(N3CCOC[C@@](C)(O)C3)c2c2ccnc(-c3c(C4(F)CC4)c(Cl)cc4[nH]ncc34)c21] | Column D, 45% MeOH w/ 0.1% NH₄OH, Second eluting isomer | (6S)-4-(8-(6-chloro- 5-(1- fluorocyclopropyl)- 1H-indazol-4-yl)-2- (((2R,7aS)-2- fluorotetrahydro- 1H-pyrrolizin- 7a(5H)- yl)methoxy)-9- methyl-9H-pyrido [4′,3′:4,5]pyrrolo [2,3-d]pyrimidin- 4-yl)-6-methyl-1,4- oxazepan-6-ol | 678 |

### Example 4:4-(2-(((2R,7aS)-2-fluorotetrahydro-1H-pyrrolizin-7a(5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)-6-methyl-5-(trifluoromethyl)-1H-indazole-3-carbonitrile (Ex-4)

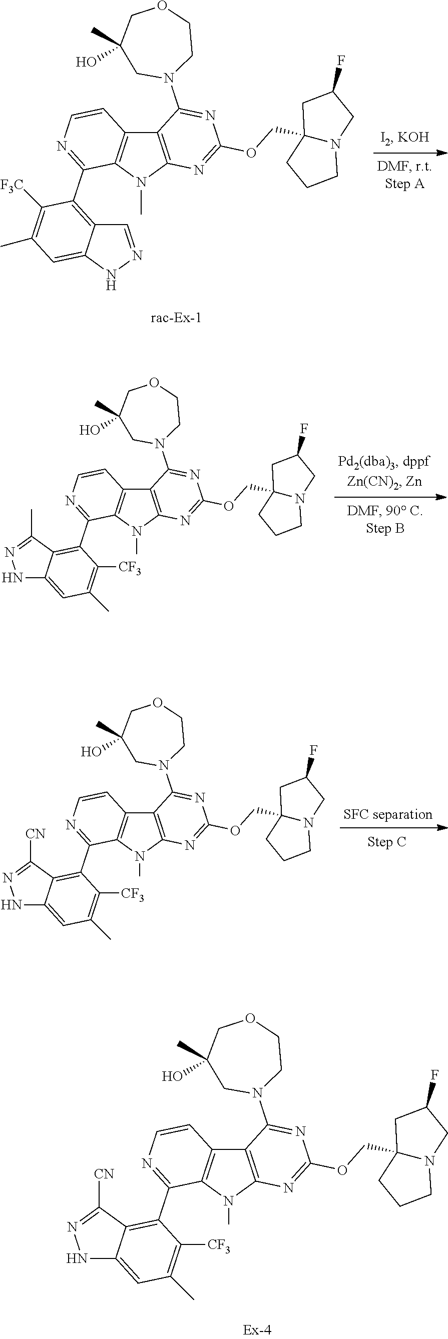 [SMILES: Cc1cc2[nH]ncc2c(-c2nccc3c4c(N5CCOC[C@@](C)(O)C5)nc(OC[C@@]56CCCN5C[C@H](F)C6)nc4n(C)c23)c1C(F)(F)F ; Cc1cc2[nH]nc(C)c2c(-c2nccc3c4c(N5CCOC[C@@](C)(O)C5)nc(OC[C@@]56CCCN5C[C@H](F)C6)nc4n(C)c23)c1C(F)(F)F ; Cc1cc2[nH]nc(C#N)c2c(-c2nccc3c4c(N5CCOC[C@@](C)(O)C5)nc(OC[C@@]56CCCN5C[C@H](F)C6)nc4n(C)c23)c1C(F)(F)F ; Cc1cc2[nH]nc(C#N)c2c(-c2nccc3c4c(N5CCOC[C@@](C)(O)C5)nc(OC[C@@]56CCCN5C[C@H](F)C6)nc4n(C)c23)c1C(F)(F)F]

### Step A: (6S)-4-(2-(((2R,7aS)-2-fluorotetrahydro-1H-pyrrolizin-7a(5H)-yl)methoxy)-8-(3-iodo-6-methyl-5-(trifluoromethyl)-1H-indazol-4-yl)-9-methyl-9H-pyrido[4′,3′:4,5]pyrrolo[2,3-d]pyrimidin-4-yl)-6-methyl-1,4-oxazepan-6-ol

To a solution of rac (6S)-4-(2-(((2R,7aS)-2-fluorotetrahydro-1H-pyrrolizin-7a(5H)-yl)methoxy)-9-methyl-8-(6-methyl-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol (rac-Ex-1) (120 mg, 0.179 mmol, mixture of atropisomers) in DMF (1.5 mL) was added KOH (30.2 mg, 0.538 mmol) and I₂ (182 mg, 0.718 mmol), the reaction was stirred at r.t. for 30 min. LCMS showed the reaction was complete. The reaction was diluted with EtOAc (15 mL), washed with brine (3×2 mL), and the organic layer was concentrated in vacuo. The residue was purified by preparative TLC (SiO₂, DCM/7M NH₃ in MeOH)=10/1) to give (6S)-4-(2-(((2R,7aS)-2-fluorotetrahydro-1H-pyrrolizin-7a(5H)-yl)methoxy)-8-(3-iodo-6-methyl-5-(trifluoromethyl)-1H-indazol-4-yl)-9-methyl-9H-pyrido[4′,3′:4,5]pyrrolo[2,3-d]pyrimidin-4-yl)-6-methyl-1,4-oxazepan-6-ol. MS (ESI) [M+H]⁺: m/z 795.

### Step B: rac 4-(2-(((2R,7aS)-2-fluorotetrahydro-1H-pyrrolizin-7a(5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)-6-methyl-5-(trifluoromethyl)-1H-indazole-3-carbonitrile

To a mixture of (6S)-4-(2-(((2R,7aS)-2-fluorotetrahydro-1H-pyrrolizin-7a(5H)-yl)methoxy)-8-(3-iodo-6-methyl-5-(trifluoromethyl)-1H-indazol-4-yl)-9-methyl-9H-pyrido[4′,3′:4,5]pyrrolo[2,3-d]pyrimidin-4-yl)-6-methyl-1,4-oxazepan-6-ol (110 mg, 0.138 mmol) in DMF (1 mL) was added Pd₂(dba) 3 (25.4 mg, 0.028 mmol), dppf (30.7 mg, 0.055 mmol), zinc cyanide (48.8 mg, 0.415 mmol), and zinc (18.10 mg, 0.277 mmol) in a glovebox. The reaction was stirred at 90° C. for 0.5 h. LCMS showed the staring material was consumed and the desired MS was found. The reaction was diluted with EtOAc (20 mL), washed with brine (3×2 mL), the organic layer was dried, and concentrated in vacuo. The residue was purified by preparative TLC (SiO₂, DCM:MeOH=10:1) to give rac 4-(2-(((2R,7aS)-2-fluorotetrahydro-1H-pyrrolizin-7a(5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)-6-methyl-5-(trifluoromethyl)-1H-indazole-3-carbonitrile. MS (ESI) [M+H]⁺: m/z 694.

### Step C: 4-(2-(((2R,7aS)-2-fluorotetrahydro-1H-pyrrolizin-7a(5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)-6-methyl-5-(trifluoromethyl)-1H-indazole-3-carbonitrile (Ex-4)

Atropisomers of rac 4-(2-(((2R,7aS)-2-fluorotetrahydro-1H-pyrrolizin-7a(5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)-6-methyl-5-(trifluoromethyl)-1H-indazole-3-carbonitrile (60 mg, 0.086 mmol) were separated by preparative SFC (Column B, 40% IPA/H₂O, 0.1% NH₃ modifier) to give 4-(2-(((2R,7aS)-2-fluorotetrahydro-1H-pyrrolizin-7a(5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)-6-methyl-5-(trifluoromethyl)-1H-indazole-3-carbonitrile (Ex-4) (the first eluting isomer from SFC). MS (ESI) [M+H]⁺: m/z 694. ¹H NMR (400 MHz, MeOD) δ 8.46 (d, J=5.60 Hz, 1H), 7.84-8.00 (m, 2H), 5.21-5.42 (m, 1H), 4.32-4.43 (m, 2H), 4.14-4.25 (m, 2H), 3.93-4.02 (m, 2H), 3.86 (d, J=14.78 Hz, 1H), 3.69 (d, J=12.52 Hz, 1H), 3.59-3.66 (m, 1H), 3.42 (d, J=12.52 Hz, 1H), 3.25-3.31 (m, 2H), 3.23 (s, 1H), 3.18 (s, 3H), 3.00-3.11 (m, 1H), 2.77 (br s, 3H), 2.27-2.39 (m, 1H), 2.19-2.27 (m, 1H), 2.09-2.18 (m, 1H), 1.96-2.05 (m, 2H), 1.85-1.94 (m, 1H), 1.27 (s, 3H).

### Example 5: (3R)-1-(2-(((2R,7aS)-2-fluorotetrahydro-1H-pyrrolizin-7a(5H)-yl)methoxy)-9-methyl-8-(6-methyl-5-(trifluoromethyl)-1H-indazol-4-yl)-9H-pyrido[4′,3′:4,5]pyrrolo[2,3-d]pyrimidin-4-yl)-3-methylpiperidin-3-ol (Ex-5)

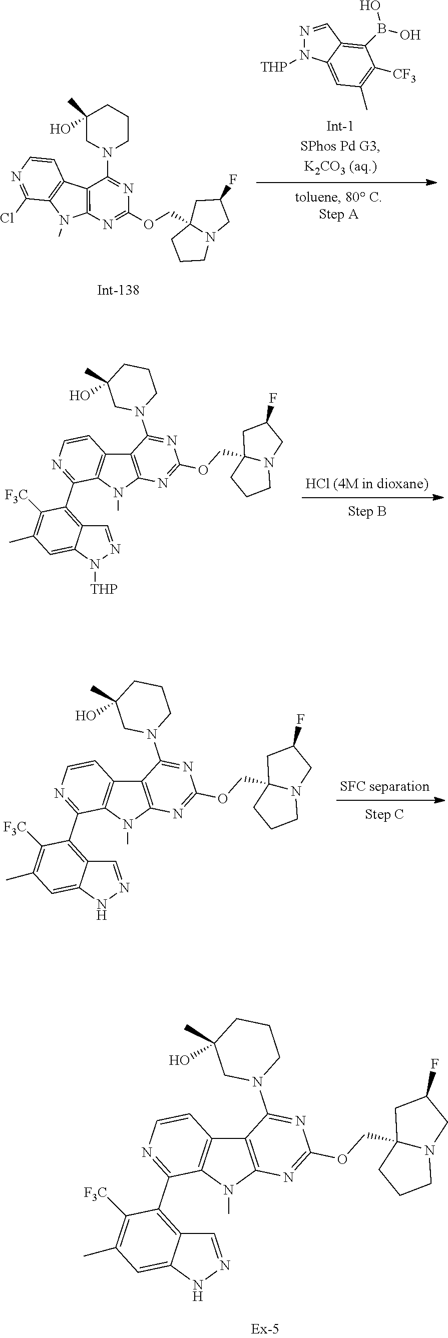

### Step A: (3R)-1-(2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-3-methylpiperidin-3-ol

To a solution of (R)-1-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-3-methylpiperidin-3-ol (Int-138) (300 mg, 0.614 mmol) in toluene (24 mL) was added (6-methyl-1-(tetrahydro-2H-pyran-2-yl)-5-(trifluoromethyl)-1H-indazol-4-yl)boronic acid (Int-1) (403 mg, 1.23 mmol), SPhos Pd G3 (239 mg, 0.301 mmol), and K₂CO₃ (0.920 mL, 1.841 mmol, 2 M in H₂O) at 25° C. The reaction was stirred at 80° C. for 3 h under N₂ atmosphere. The reaction was diluted with EtOAc (30 mL) and washed with brine (10 mL). The organic layer was dried over sodium sulfate, filtered, and concentrated in vacuo. The residue was purified by flash silica gel chromatography (0-50% THF in petroleum ether) to give (3R)-1-(2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-3-methylpiperidin-3-ol. MS (ESI) [M+H]⁺: m/z 737.

### Step B: Rac (3R)-1-(2-(((2R,7aS)-2-fluorotetrahydro-1H-pyrrolizin-7a(5H)-yl)methoxy)-9-methyl-8-(6-methyl-5-(trifluoromethyl)-1H-indazol-4-yl)-9H-pyrido[4′,3′:4,5]pyrrolo[2,3-d]pyrimidin-4-yl)-3-methylpiperidin-3-ol

A mixture of (3R)-1-(2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-3-methylpiperidin-3-ol (100 mg, 0.136 mmol) and HCl (2 mL, 4 M in dioxane) was stirred at 25° C. for 1 h. The reaction mixture was concentrated in vacuo to give a crude product, which was diluted with EtOAc (5 mL) and basified with sat. aq. NaHCO₃ solution (5 mL). The resulting mixture was extracted with EtOAc (10 mL) and washed with brine (3×2 mL). The combined organic phase was dried over anhydrous Na₂SO₄, filtered, and concentrated in vacuo to give a crude product, which was purified by preparative TLC (silica gel, DCM/MeOH=7:1) to give rac (3R)-1-(2-(((2R,7aS)-2-fluorotetrahydro-1H-pyrrolizin-7a(5H)-yl)methoxy)-9-methyl-8-(6-methyl-5-(trifluoromethyl)-1H-indazol-4-yl)-9H-pyrido[4′,3′:4,5]pyrrolo[2,3-d]pyrimidin-4-yl)-3-methylpiperidin-3-ol. MS (ESI) [M+H]⁺: m/z 653.

### Step C: (3R)-1-(2-(((2R,7aS)-2-fluorotetrahydro-1H-pyrrolizin-7a(5H)-yl)methoxy)-9-methyl-8-(6-methyl-5-(trifluoromethyl)-1H-indazol-4-yl)-9H-pyrido[4′,3′:4,5]pyrrolo[2,3-d]pyrimidin-4-yl)-3-methylpiperidin-3-ol (Ex-5)

Atropisomers of rac (3R)-1-(2-(((2R,7aS)-2-fluorotetrahydro-1H-pyrrolizin-7a(5H)-yl)methoxy)-9-methyl-8-(6-methyl-5-(trifluoromethyl)-1H-indazol-4-yl)-9H-pyrido[4′,3′:4,5]pyrrolo[2,3-d]pyrimidin-4-yl)-3-methylpiperidin-3-ol (95 mg, 0.15 mmol) were separated by preparative SFC (Column C, 55% EtOH/H₂O, 0.1% NH₄OH modifier) to give (3R)-1-(2-(((2R,7aS)-2-fluorotetrahydro-1H-pyrrolizin-7a(5H)-yl)methoxy)-9-methyl-8-(6-methyl-5-(trifluoromethyl)-1H-indazol-4-yl)-9H-pyrido[4′,3′:4,5]pyrrolo[2,3-d]pyrimidin-4-yl)-3-methylpiperidin-3-ol (Ex-5) (the second eluting isomer by SFC). MS (ESI) [M+H]⁺: m/z 653. ¹H NMR (400 MHz, CDCl₃) δ 8.48 (d, J=5.36 Hz, 1H), 7.74 (d, J=5.36 Hz, 1H), 7.21 (br d, J=6.91 Hz, 2H), 5.07-5.32 (m, 1H), 3.90-4.49 (m, 5H), 3.16-3.41 (m, 3H), 3.04-3.14 (m, 2H), 2.99 (s, 3H), 2.87-2.95 (m, 1H), 2.55 (br s, 3H), 2.03-2.24 (m, 3H), 1.75-1.97 (m, 5H), 1.51-1.70 (m, 2H), 1.28 (s, 3H).

The example in the table below was synthesized using a similar procedure as described in the synthesis of Ex-5 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | SFC | | |
|---|---|---|---|---|---|
| | | | Sepa- | | |
| | | | ration | | |
| | Starting | | Con- | Compound | [M + H]⁺ |
| Ex. | Material | Structure | ditions | Name | Found |
| Ex- 6 | 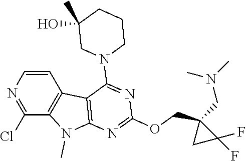 [SMILES: CN(C)C[C@@]1(COc2nc(N3CCC[C@@](C)(O)C3)c3c4ccnc(Cl)c4n(C)c3n2)CC1(F)F] Int-139 | 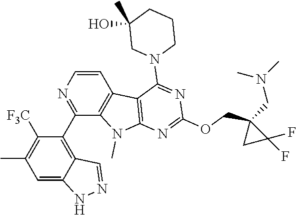 [SMILES: Cc1cc2[nH]ncc2c(-c2nccc3c4c(N5CCC[C@@](C)(O)C5)nc(OC[C@]5(CN(C)C)CC5(F)F)nc4n(C)c23)c1C(F)(F)F] | Column C, 35% MeOH w/ 0.1% NH₄OH | (3R)-1-(2- (((R)-1-((di- methylamino) methyl)-2,2- difluoro- cyclopropyl) methoxy)-9- methyl-8-(6- methyl-5- (trifluoro- methyl)- 1H-indazol- 4-yl)- | 659 |
| | | | | 9H- | |
| | | | | pyrido | |
| | | | | [4′,3′:4,5] | |
| | | | | pyrrolo[2,3- | |
| | | | | d]pyrimidin- | |
| | | | | 4-yl)-3- | |
| | | | | methyl- | |
| | | | | piperidin- | |
| | | | | 3-ol | |

### Example 7: (6S)-4-(8-(5-ethynyl-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-7)

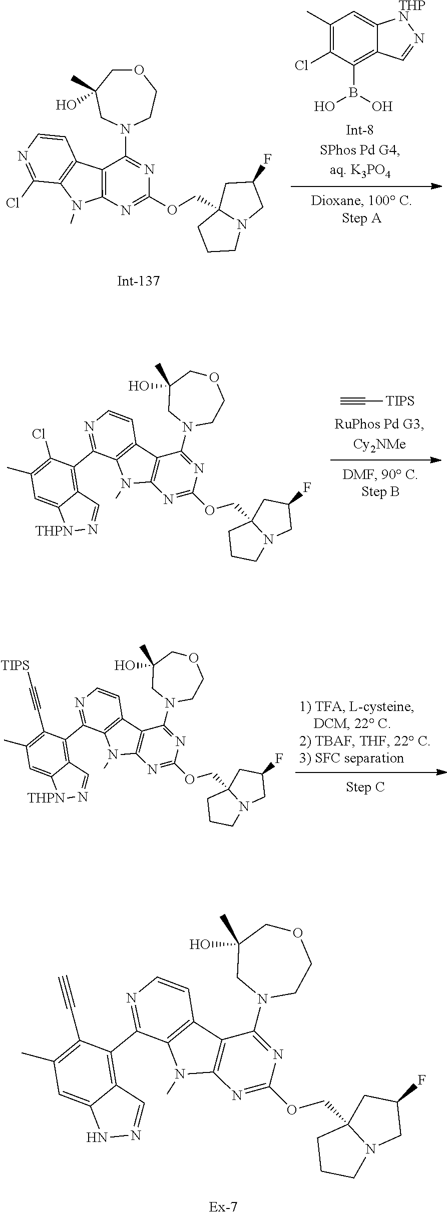

### Step A: (6S)-4-(8-(5-chloro-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol

A vial was loaded with (5-chloro-6-methyl-1-(tetrahydro-2H-pyran-2-yl)-3a,7a-dihydro-1H-indazol-4-yl)boronic acid (Int-8) (109 mg, 0.370 mmol), (S)-4-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Int-137) (106 mg, 0.209 mmol), SPhos Pd G4 (33.2 mg, 0.042 mmol), and 2 mL of dioxane under argon. A solution of potassium phosphate (2M in degassed water, 314 μL) was added via a syringe and the sample was heated to 100° C. for 20 h. The reaction was quenched with water (2 mL) and extracted with DCM (3×10 mL). The organic layer was dried over Na₂SO₄, filtered, and evaporated under reduced pressure. The mixture was purified by silica gel chromatography (17% MeOH in DCM), affording (6S)-4-(8-(5-chloro-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol. MS (ESI): m/z (M+H)⁺ 720.

### Step B: (6S)-4-(2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(triisopropylsilyl)ethynyl)-1H-indazol-4-yl)-9H-pyrido[4′,3′:4,5]pyrrolo[2,3-d]pyrimidin-4-yl)-6-methyl-1,4-oxazepan-6-ol

A vial was loaded with (6S)-4-(8-(5-chloro-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (68 mg, 0.095 mmol), RuPhos Pd G3 (8.0 mg, 9.5 mol) and DMF (1 mL) under nitrogen.

Ethynyltriisopropylsilane (212 μL, 0.945 mmol) and N-cyclohexyl-N-methylcyclohexanamine (122 μL, 0.567 mmol) were added via syringe and the mixture was heated to 90° C. for 16 hours. The organic layer was dried over Na₂SO₄, filtered, and evaporated under reduced pressure. The mixture was purified by silica gel chromatography (17% MeOH in DCM), affording (6S)-4-(2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-((triisopropylsilyl)ethynyl)-1H-indazol-4-yl)-9H-pyrido[4′,3′:4,5]pyrrolo[2,3-d]pyrimidin-4-yl)-6-methyl-1,4-oxazepan-6-ol. MS (ESI): m/z (M+H)⁺ 866.

### Step C: (6S)-4-(8-(5-ethynyl-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-7)

A vial was loaded with (6S)-4-(2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-((triisopropylsilyl)ethynyl)-1H-indazol-4-yl)-9H-pyrido[4′,3′:4,5]pyrrolo[2,3-d]pyrimidin-4-yl)-6-methyl-1,4-oxazepan-6-ol (68 mg, 0.090 mmol) and L-cysteine (10.9 mg, 0.0900 mmol). DCM (1.5 mL) and TFA (0.5 mL) was added to the mixture. The mixture was allowed to stir for 2 h. The desired product was found and no starting material remained. The volatiles were removed under reduced pressure and the remaining crude was redissolved in 3 mL of DCM and 2 mL of sat. aq. NaHCO₃. The aqueous layer was extracted with DCM (3×5 mL), dried over Na₂SO₄, filtered, and concentrated under reduced pressure. The crude mixture was used directly in the next step without further purification and was dissolved in THF (0.9 mL). Tetra-n-butylammonium fluoride (IM in THF, 0.315 mL, 0.315 mmol) was added via a syringe. The mixture was allowed to stir at r.t. for 2 hours. The mixture was purified via preparative reverse-phase HPLC (MeCN/H₂O with TFA modifier). The atropisomers were resolved by SFC purification (Column D, 35% MeOH with 0.1% NH₄OH) to afford (6S)-4-(8-(5-ethynyl-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-7) (the second eluting isomer by SFC). MS (ESI): m/z (M+H)⁺ 625. ¹H NMR (600 MHz, MeOD) δ 8.49 (d, J=5.5 Hz, 1H), 7.92 (d, J=5.6 Hz, 1H), 7.65 (s, 1H), 7.57 (s, 1H), 5.42-5.25 (m, 1H), 4.50-4.40 (m, 1H), 4.34 (d, J=10.4 Hz, 1H), 4.26-4.16 (m, 2H), 4.08-3.95 (m, 2H), 3.88 (d, J=14.8 Hz, 1H), 3.80-3.68 (m, 2H), 3.47 (d, J=12.5 Hz, 1H), 3.43 (s, 1H), 3.31-3.21 (m, 5H), 3.07-3.01 (m, 1H), 2.69 (s, 3H), 2.38-2.17 (m, 3H), 2.17-2.07 (m, 1H), 2.07-1.96 (m, 2H), 1.95-1.84 (m, 1H), 1.38-1.23 (m, 4H).

### Example 8: (6S)-4-(8-(5-(difluoromethyl)-3,6-dimethyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-8)

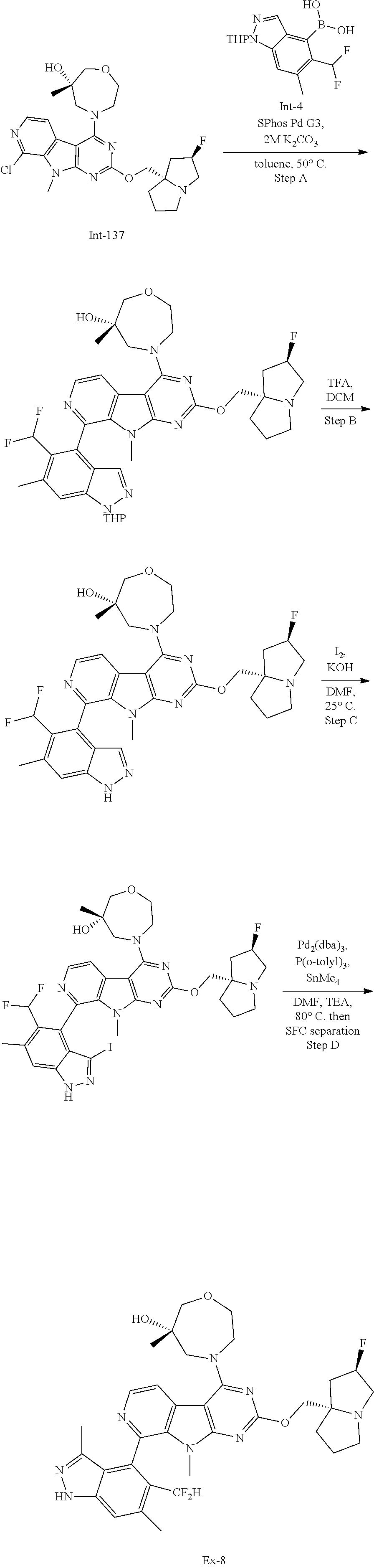

### Step A: (6S)-4-(8-(5-(difluoromethyl)-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol

To a solution of(S)-4-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Int-137) (800 mg, 1.58 mmol) in toluene (1 mL) was added (5-(difluoromethyl)-6-methyl-1-(tetrahydro-2H-pyran-2-yl)-1H-indazol-4-yl)boronic acid (Int-4) (737 mg, 2.38 mmol), SPhos Pd G3 (618 mg, 0.792 mmol), and potassium carbonate (2.38 mL, 4.75 mmol, 2 M in H₂O) at 25° C. The reaction was stirred at 50° C. for 2 h under N₂ atmosphere. The reaction was diluted with EtOAc (30 mL), washed with brine (1 mL), the organic layer was dried and concentrated in vacuo. The residue was purified by flash silica gel chromatography (0-50% THF in petroleum ether) to give (6S)-4-(8-(5-(difluoromethyl)-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol. MS (ESI) [M+H]⁺: m/z 735.

### Step B: (6S)-4-(8-(5-(difluoromethyl)-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol

A solution of (6S)-4-(8-(S-(difluoromethyl)-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (800 mg, 1.09 mmol) in DCM (8 mL) was added TFA (2.67 mL, 34.7 mmol) at 25° C. The reaction was stirred at 25° C. for 1.5 h. The reaction mixture was concentrated in vacuo, and the residue was diluted with EtOAc (20 mL), basified with aqueous NaHCO₃ to pH 8, and separated. The organic layer was dried over sodium sulfate, filtered, and concentrated in vacuum to give (6S)-4-(8-(5-(difluoromethyl)-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol. MS (ESI) [M+H]⁺: m/z 651.

### Step C: (6S)-4-(8-(5-(difluoromethyl)-3-iodo-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol

To a solution of (6S)-4-(8-(5-(difluoromethyl)-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (714 mg, 1.10 mmol) in DMF (8 mL) was added KOH (185 mg, 3.29 mmol) and 12 (1.11 g, 4.39 mmol). The reaction was stirred at 25° C. for 30 min. The reaction mixture was diluted with H₂O (5 mL), and then quenched with 2M Na₂SO₃ (10 mL), and extracted with EtOAc (60 mL). The organic phase was dried over Na₂SO₄, filtered, and concentrated in vacuo to give a crude product which was purified by flash silica gel chromatography (petroleum ether/THF=1/2) to give (6S)-4-(8-(5-(difluoromethyl)-3-iodo-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol. MS (ESI) [M+H]⁺: m/z 777.

### Step D: (6S)-4-(8-(5-(difluoromethyl)-3,6-dimethyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-8)

To a mixture of (6R)-4-(8-(5-(difluoromethyl)-3-iodo-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (223 mg, 0.287 mmol) in DMF (2 mL) was added tri-o-tolylphosphine (17 mg, 0.057 mmol), tris(dibenzylideneacetone) dipalladium (0) (26 mg, 0.029 mmol), tetramethylstannane (128 mg, 0.718 mmol), and triethylamine (0.120 mL, 0.861 mmol). The reaction was stirred at 80° C. for 3 h under nitrogen. The reaction mixture was diluted with MeCN (2 mL), and filtered. The filtrate was purified by preparative reverse-phase HPLC (MeCN/H₂O w/10 mM NH₄HCO₃ modifier). The atropisomers were separated via preparative SFC (Column B, 40% MeOH w/0.1% NH₄OH) to give (6S)-4-(8-(S-(difluoromethyl)-3,6-dimethyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-8) (the first eluting isomer from SFC). MS (ESI) [M+H]⁺: m/z 665. ¹H NMR (400 MHz, MeOD) δ 8.39 (d, J=5.6 Hz, 1H), 7.86 (d, J=5.6 Hz, 1H), 7.48 (s, 1H), 6.56-6.21 (m, 1H), 5.31-5.08 (m, 1H), 4.33-4.18 (m, 2H), 4.14-4.06 (m, 2H), 3.99-3.85 (m, 2H), 3.79 (d, J=14.7 Hz, 1H), 3.74-3.66 (m, 1H), 3.59 (d, J=12.5 Hz, 1H), 3.37 (d, J=12.5 Hz, 1H), 3.19-3.08 (m, 3H), 3.01 (s, 3H), 2.95-2.87 (m, 1H), 2.63 (s, 3H), 2.30-2.07 (m, 2H), 2.05-1.95 (m, 1H), 1.94-1.74 (m, 3H), 1.38 (s, 3H), 1.13 (s, 3H).

### Example 9: 5-(difluoromethyl)-4-(2-(((2R,7aS)-2-fluorotetrahydro-1H-pyrrolizin-7a(5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)-6-methyl-1H-indazole-3-carbonitrile (Ex-9)

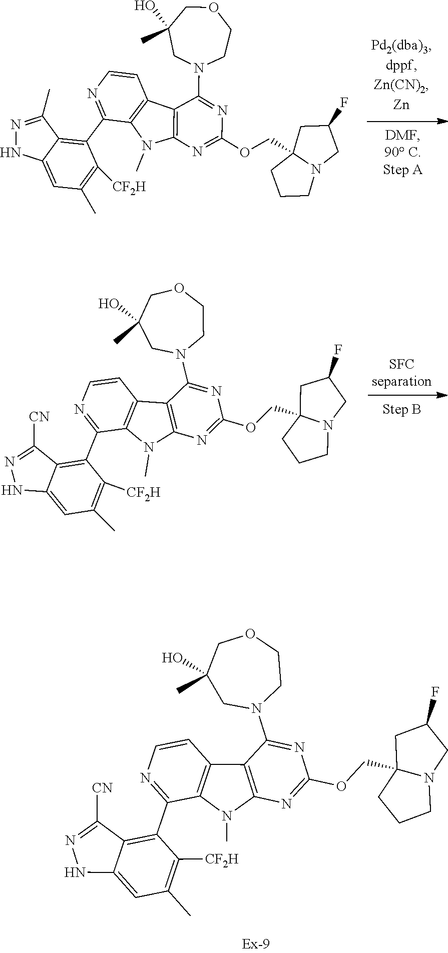 [SMILES: Cc1cc2[nH]nc(C)c2c(-c2nccc3c4c(N5CCOC[C@@](C)(O)C5)nc(OC[C@@]56CCCN5C[C@H](F)C6)nc4n(C)c23)c1C(F)F ; Cc1cc2[nH]nc(C#N)c2c(-c2nccc3c4c(N5CCOC[C@@](C)(O)C5)nc(OC[C@@]56CCCN5C[C@H](F)C6)nc4n(C)c23)c1C(F)F ; Cc1cc2[nH]nc(C#N)c2c(-c2nccc3c4c(N5CCOC[C@@](C)(O)C5)nc(OC[C@@]56CCCN5C[C@H](F)C6)nc4n(C)c23)c1C(F)F]

### Step A: rac 5-(difluoromethyl)-4-(2-(((2R,7aS)-2-fluorotetrahydro-1H-pyrrolizin-7a(5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)-6-methyl-1H-indazole-3-carbonitrile

To a mixture of (6S)-4-(8-(5-(difluoromethyl)-3-iodo-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (from Step C of Example 6) (150 mg, 0.193 mmol) in DMF (3 mL) was added zinc (25.3 mg, 0.386 mmol), zinc cyanide (113 mg, 0.966 mmol), 1,1′-bis(diphenylphosphino)ferrocene (21 mg, 0.039 mmol), and tris(dibenzylideneacetone) dipalladium (0) (18 mg, 0.019 mmol) at 25° C. The reaction was stirred at 90° C. for 30 min under nitrogen atmosphere. The reaction mixture was diluted with EtOAc (20 mL), and the reaction mixture was filtered. The organic phase was washed with H₂O (3 mL) and brine (3 mL×2). The combined organic phase was dried over anhydrous Na₂SO₄, filtered, and concentrated in vacuo. The crude material was purified by preparative reverse-phase HPLC (MeCN/H₂O w/0.1% TFA modifier) to obtain to give rac 5-(difluoromethyl)-4-(2-(((2R,7aS)-2-fluorotetrahydro-1H-pyrrolizin-7a(5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)-6-methyl-1H-indazole-3-carbonitrile. MS (ESI) [M+H]⁺: m/z 676.

### Step B: 5-(difluoromethyl)-4-(2-(((2R,7aS)-2-fluorotetrahydro-1H-pyrrolizin-7a(5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)-6-methyl-1H-indazole-3-carbonitrile (Ex-9)

Atropisomers of rac 5-(difluoromethyl)-4-(2-(((2R,7aS)-2-fluorotetrahydro-1H-pyrrolizin-7a(5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)-6-methyl-1H-indazole-3-carbonitrile were separated by preparative SFC (Column B, 35% EtOH w/0.1% NH₃) to give 5-(difluoromethyl)-4-(2-(((2R,7aS)-2-fluorotetrahydro-1H-pyrrolizin-7a(5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)-6-methyl-1H-indazole-3-carbonitrile (Ex-9) (the first eluting isomer from SFC). MS (ESI) [M+H]⁺: m/z 676. ¹H NMR (400 MHz, MeOD) δ 8.39 (d, J=5.5 Hz, 1H), 7.82 (d, J=5.6 Hz, 1H), 7.69 (s, 1H), 6.83-6.46 (m, 1H), 5.30-5.09 (m, 1H), 4.32-4.20 (m, 2H), 4.13-4.01 (m, 2H), 3.91-3.79 (m, 2H), 3.73 (d, J=14.7 Hz, 1H), 3.61-3.50 (m, 2H), 3.31 (d, J=12.5 Hz, 1H), 3.19-3.08 (m, 3H), 3.00 (s, 3H), 2.94-2.85 (m, 1H), 2.67 (br s, 3H), 2.27-1.74 (m, 6H), 1.15 (s, 3H).

### Example 10: (6S)-4-(8-(5-(difluoromethyl)-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-10)

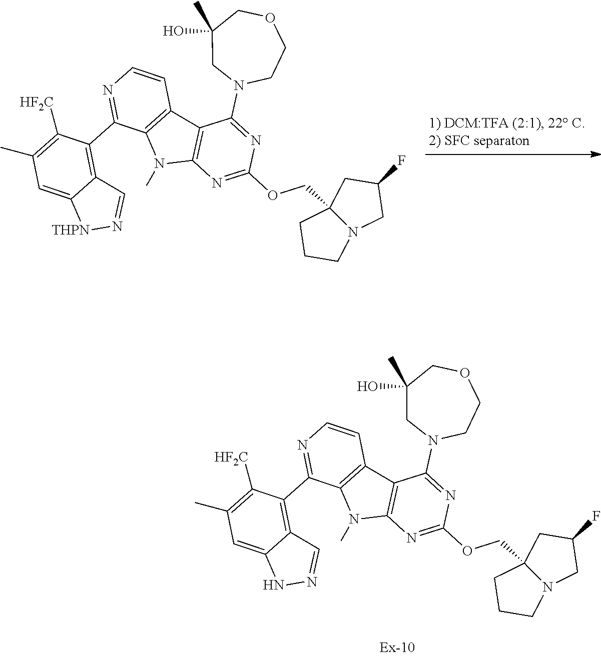

A vial was loaded with (6S)-4-(8-(5-(difluoromethyl)-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (from Step A of Example 6) (121 mg, 0.17 mmol) and was dissolved DCM (2 mL) and TFA (1 mL). The reaction was monitored by LCMS. After 30 minutes, the reaction was completed. The volatiles were evaporated under reduced pressure, and the residue was dissolved in DCM (5 mL). The solution was basified with Na₂CO₃ and extracted with DCM (3×5 mL). The volatiles were evaporated under reduced pressure. The material was purified by SFC (20% MeOH with 0.1% NH₄OH). The atropisomers were resolved by SFC purification (Column D, 35% MeOH with 0.1% NH₄OH) to afford (6S)-4-(8-(5-(difluoromethyl)-6-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-10) (the second eluting isomer from SFC). MS (ESI): m/z (M+H)⁺ 651. ¹H NMR (600 MHz, DMSO-d₆) δ 8.50 (d, J=5.2 Hz, 1H), 7.86 (d, J=5.4 Hz, 1H), 7.66 (s, 1H), 7.42 (s, 1H), 6.70 (t, J=53.6 Hz, 1H), 5.49 (s, 1H), 5.35-5.14 (m, 1H), 4.21-3.96 (m, 7H), 3.88 (s, 1H), 3.67-3.50 (m, 1H), 3.48-3.43 (m, 1H), 3.14-2.99 (m, 2H), 2.95 (s, 3H), 2.82 (s, 1H), 2.71 (s, 3H), 2.16-1.92 (m, 3H), 1.91-1.67 (m, 5H), 1.10 (s, 3H).

### Example 11: (6S)-4-(8-(6-chloro-5-(difluoromethyl)-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-11)

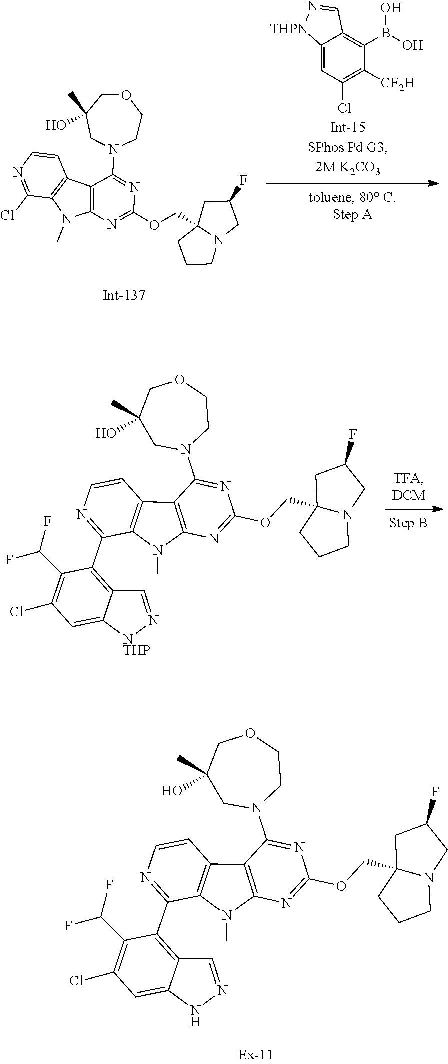

### Step A: (6S)-4-(8-(6-chloro-5 (difluoromethyl)-1-(tetrahydro-2H-pyran-2-yl)-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol

To a solution of(S)-4-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Int-137) (1.2 g, 2.4 mmol) in toluene (17 mL) was added (6-chloro-5-(difluoromethyl)-1-(tetrahydro-2H-pyran-2-yl)-1H-indazol-4-yl)boronic acid (Int-15) (0.785 g, 2.38 mmol), SPhos Pd G3 (0.927 g, 1.19 mmol), and potassium carbonate (3.56 mL, 7.13 mmol, 2M in H₂O) at 25° C. under N₂ atmosphere. The reaction was stirred at 80° C. for 16 h. LCMS showed the reaction was finished. The reaction was diluted with EtOAc (30 mL) and washed with brine (10 mL). The organic layer was dried over sodium sulfate, filtered, and concentrated in vacuo. The residue was purified by flash silica gel chromatography (0 to 50% THF in petroleum ether) to give (6S)-4-(8-(6-chloro-5-(difluoromethyl)-1-(tetrahydro-2H-pyran-2-yl)-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol. MS (ESI) [M+H]⁺: m/z 755.

### Step B: (6S)-4-(8-(6-chloro-5-(difluoromethyl)-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepam-6-ol (Ex-11)

To a solution of (6S)-4-(8-(6-chloro-5-(difluoromethyl)-1-(tetrahydro-2H-pyran-2-yl)-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (500 mg, 0.66 mmol) in DCM (5 mL) was added TFA (1.67 mL, 21.68 mmol). The reaction mixture was stirred at 25° C. for 2 h. LCMS showed the reaction was finished. Then the reaction was concentrated in vacuo and the residue was purified by reverse-phase MPLC (0 to 40% MeCN in H₂O, 0.05% TFA) to give rac (6S)-4-(8-(6-chloro-5-(difluoromethyl)-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (rac-Ex-11). The atropisomers were separated by preparative SFC (Column E, 40% MeOH w/0.1% NH₄OH). The separated atropisomers were further purified by preparative HPLC (MeCN/H₂O w/NH₄OH modifier) to give (6S)-4-(8-(6-chloro-5-(difluoromethyl)-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-11) (the second eluting isomer from SFC). MS (ESI) [M+H]⁺: m/z 671. ¹H NMR (400 MHz, MeOD) δ 8.49 (d, J=5.60 Hz, 1H), 7.91-7.97 (m, 2H), 7.53 (s, 1H), 6.74-7.06 (m, 1H), 5.26-5.37 (m, 1H), 4.43 (br d, J=14.78 Hz, 1H), 4.29 (d, J=10.37 Hz, 1H), 4.14-4.22 (m, 2H), 3.93-4.06 (m, 2H), 3.86 (d, J=14.90 Hz, 1H), 3.66-3.77 (m, 2H), 3.45 (d, J=12.28 Hz, 1H), 3.23 (br s, 2H), 3.17 (br s, 1H), 3.15 (s, 3H), 3.01 (br s, 1H), 2.16-2.35 (m, 2H), 2.05-2.15 (m, 1H), 1.94-2.02 (m, 2H), 1.87 (br s, 1H), 1.29 (s, 3H).

### Example 12:6-chloro-5-(difluoromethyl)-4-(2-(((2R,7aS)-2-fluorotetrahydro-1H-pyrrolizin-7a(5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)-1H-indazole-3-carbonitrile (Ex-12)

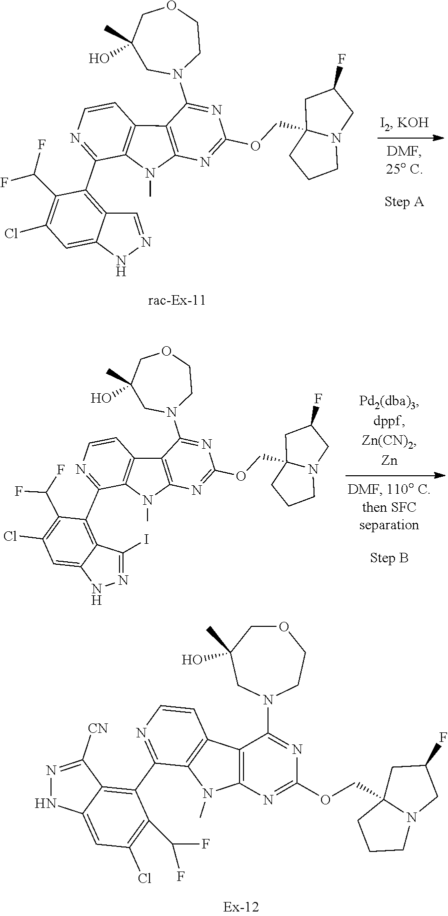 [SMILES: Cn1c2nc(OC[C@@]34CCCN3C[C@H](F)C4)nc(N3CCOC[C@@](C)(O)C3)c2c2ccnc(-c3c(C(F)F)c(Cl)cc4[nH]ncc34)c21 ; Cn1c2nc(OC[C@@]34CCCN3C[C@H](F)C4)nc(N3CCOC[C@@](C)(O)C3)c2c2ccnc(-c3c(C(F)F)c(Cl)cc4[nH]nc(I)c34)c21 ; Cn1c2nc(OC[C@@]34CCCN3C[C@H](F)C4)nc(N3CCOC[C@@](C)(O)C3)c2c2ccnc(-c3c(C(F)F)c(Cl)cc4[nH]nc(C#N)c34)c21]

### Step A: (6S)-4-(8-(6-chloro-5-(difluoromethyl)-3-iodo-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol

To a solution of rac (6S)-4-(8-(6-chloro-5-(difluoromethyl)-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (rac-Ex-11) (200 mg, 0.306 mmol, mixture of atropisomers) in DMF (3 mL) was added KOH (51.5 mg, 0.919 mmol) and 12 (311 mg, 1.23 mmol). The reaction mixture was stirred at 25° C. for 30 min. LCMS showed the reaction was finished. The reaction mixture was quenched with 2M Na₂SO₃ solution (5 mL), and extracted with EtOAc (3×15 mL). The combined organic phase was dried over anhydrous Na₂SO₄, filtered, and concentrated in vacuo. The crude material was purified by preparative HPLC (MeCN/H₂O w/TFA modifier) to give (6S)-4-(8-(6-chloro-5-(difluoromethyl)-3-iodo-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol. MS (ESI) [M+H]⁺: m/z 797.

### Step B: 6-chloro-5-(difluoromethyl)-4-(2-(((2R,7aS)-2-fluorotetrahydro-1H-pyrrolizin-7a(5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)-1H-indazole-3-carbonitrile (Ex-12)

To a mixture of (6S)-4-(8-(6-chloro-5-(difluoromethyl)-3-iodo-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (65 mg, 0.082 mmol) in DMF (0.8 mL) was added zinc (10.66 mg, 0.163 mmol), zinc cyanide (47.9 mg, 0.408 mmol), dppf (9.04 mg, 0.016 mmol), and tris(dibenzylidencacetone) dipalladium (0) (7.47 mg, 8.16 μmol) at 25° C. The reaction was stirred at 110° C. for 30 min under nitrogen atmosphere. LCMS showed the starting material was consumed and the desired mass was formed. The reaction mixture was diluted with EtOAc (20 mL) and filtered. The filtrate was washed with brine (2×2 mL). The organic layer was dried over anhydrous Na₂SO₄, filtered, and concentrated in vacuo. The residue was purified by preparative reverse-phase HPLC (MeCN/H₂O w/NH₃ modifier). The atropisomers were separated by preparative SFC (Column B, 35% IPA w/0.1% NH₃). The separated atropisomers were further purified by preparative HPLC (MeCN/H₂O w/NH₃ modifier) to give 6-chloro-5-(difluoromethyl)-4-(2-(((2R,7aS)-2-fluorotetrahydro-1H-pyrrolizin-7a(5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)-1H-indazole-3-carbonitrile (Ex-12) (the first eluting isomer from SFC). MS (ESI) [M+H]⁺: m/z 696. ¹H NMR (400 MHz, MeOD) δ8.51 (d, J=5.48 Hz, 1H), 8.13 (s, 1H), 7.97 (d, J=5.60 Hz, 1H), 6.89-7.19 (m, 1H), 5.34-5.52 (m, 1H), 4.46-4.54 (m, 1H), 4.33-4.41 (m, 2H), 4.20 (d, J=14.78 Hz, 1H), 3.93-4.03 (m, 2H), 3.88 (d, J=14.78 Hz, 1H), 3.65-3.75 (m, 2H), 3.51-3.58 (m, 2H), 3.40-3.51 (m, 2H), 3.20-3.26 (m, 1H), 3.18 (s, 3H), 2.31-2.56 (m, 2H), 2.21-2.28 (m, 1H), 2.09-2.19 (m, 2H), 1.97-2.07 (m, 1H), 1.26 (s, 3H).

### Example 13: (6S)-4-(8-(6-chloro-5-(difluoromethyl)-3-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-13)

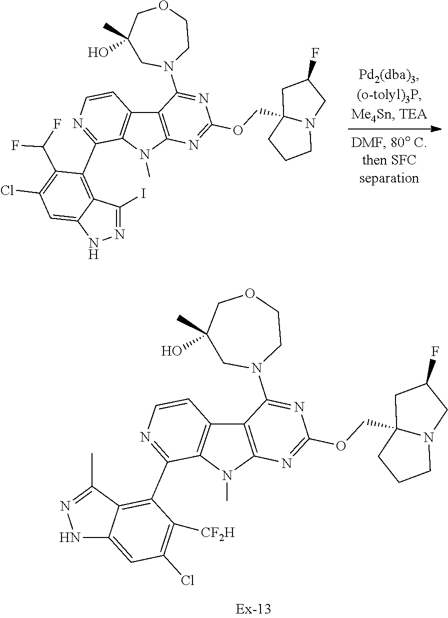 [SMILES: Cn1c2nc(OC[C@@]34CCCN3C[C@H](F)C4)nc(N3CCOC[C@@](C)(O)C3)c2c2ccnc(-c3c(C(F)F)c(Cl)cc4[nH]nc(I)c34)c21 ; Cc1n[nH]c2cc(Cl)c(C(F)F)c(-c3nccc4c5c(N6CCOC[C@@](C)(O)C6)nc(OC[C@@]67CCCN6C[C@H](F)C7)nc5n(C)c34)c12]

To a mixture of (6S)-4-(8-(6-chloro-5-(difluoromethyl)-3-iodo-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (from Step A of Example 10) (45 mg, 0.056 mmol) in DMF (1 mL) was added tri-o-tolylphosphine (3.44 mg, 0.011 mmol), tris(dibenzylideneacetone) dipalladium (0) (5.17 mg, 5.65 μmol), tetramethylstannane (25.2 mg, 0.141 mmol), and triethylamine (0.024 mL, 0.169 mmol). The reaction was stirred at 80° C. for 3 h under nitrogen atmosphere. LCMS showed that the starting material disappeared and the desired mass was found. The reaction mixture was concentrated in vacuo to give a crude product which was purified by preparative reverse-phase HPLC (MeCN/H₂O w/NHs modifier). The atropisomers were separated by preparative SFC (Column B, 40% IPA w/0.1% NH₃) to give (6S)-4-(8-(6-chloro-5-(difluoromethyl)-3-methyl-1H-indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-13) (the first eluting isomer from SFC). MS (ESI) [M+H]⁺: m/z 685. ¹H NMR (400 MHz, MeOD) δ 8.49 (d, J=5.48 Hz, 1H), 7.97 (d, J=5.60 Hz, 1H), 7.87 (s, 1H), 6.68-7.00 (m, 1H), 5.18-5.40 (m, 1H), 4.34-4.42 (m, 1H), 4.25-4.31 (m, 1H), 4.14-4.23 (m, 2H), 3.96-4.08 (m, 2H), 3.90 (d, J=14.78 Hz, 1H), 3.75-3.83 (m, 1H), 3.69 (d, J=12.40 Hz, 1H), 3.48 (d, J=12.40 Hz, 1H), 3.19-3.26 (m, 2H), 3.17 (s, 3H), 3.15 (s, 1H), 2.99 (dt, J=5.60, 9.36 Hz, 1H), 2.21-2.35 (m, 1H), 2.15-2.20 (m, 1H), 2.04-2.11 (m, 1H), 1.93-2.02 (m, 2H), 1.85-1.90 (m, 1H), 1.49 (s, 3H), 1.24 (s, 3H).

### Example 14: (6S)-4-(2-(((2R,7aS)-2-fluorotetrahydro-1H-pyrrolizin-7a(5H)-yl)methoxy)-9-methyl-8-(6-methyl-5-(trifluoromethyl)-1H-pyrazolo[3,4-b]pyridin-4-yl)-9H-pyrido[4′,3′:4,5]pyrrolo[2,3-d]pyrimidin-4-yl)-6-methyl-1,4-oxazepan-6-ol (Ex-14)

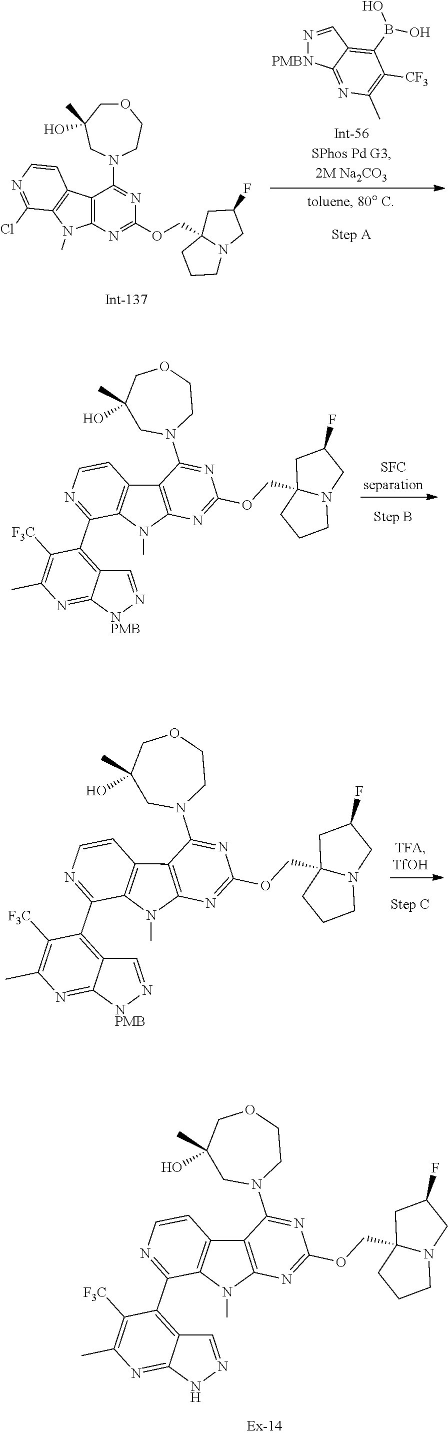

### Step A: Rac (6S)-4-(2-(((2R,7aS)-2-fluorotetrahydro-1H-pyrrolizin-7a(5H)-yl)methoxy)-8-(1-(4-methoxybenzyl)-6-methyl-5-(trifluoromethyl)-1H-pyrazolo[3,4-b]pyridin-4-yl)-9-methyl-9H-pyrido[4′,3′:4,5]pyrrolo[2,3-d]pyrimidin-4-yl)-6-methyl-1,4-oxazepan-6-ol

To a solution of(S)-4-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Int-137) (150 mg, 0.297 mmol) in toluene (6 mL) was added (1-(4-methoxybenzyl)-6-methyl-5-(trifluoromethyl)-1H-pyrazolo[3,4-b]pyridin-4-yl)boronic acid (Int-56) (217 mg, 0.594 mmol), SPhos Pd G3 (116 mg, 0.149 mmol), and 2M aqueous Na₂CO₃ (0.446 mL, 0.891 mmol) in a glovebox. The reaction was divided into three parts. The reaction mixtures were stirred at 80° C. for 1 h. LCMS showed the desired mass was formed. The reaction solutions were cooled, combined, evaporated under reduced pressure to give a crude product. The crude product was purified by preparative TLC (SiO₂, DCM:MeOH=10:1) to give rac (6S)-4-(2-(((2R,7aS)-2-fluorotetrahydro-1H-pyrrolizin-7a(5H)-yl)methoxy)-8-(1-(4-methoxybenzyl)-6-methyl-5-(trifluoromethyl)-1H-pyrazolo[3,4-b]pyridin-4-yl)-9-methyl-9H-pyrido[4′,3′:4,5]pyrrolo[2,3-d]pyrimidin-4-yl)-6-methyl-1,4-oxazepan-6-ol. MS (ESI) [M+H]⁺: m/z 790.

### Step B: (6S)-4-(2-(((2R,7aS)-2-fluorotetrahydro-1H-pyrrolizin-7a(5H)-yl)methoxy)-8-(1-(4-methoxybenzyl)-6-methyl-5-(trifluoromethyl)-1H-pyrazolo[3,4-b]pyridin-4-yl)-9-methyl-9H-pyrido[4′,3′:4,5]pyrrolo[2,3-d]pyrimidin-4-yl)-6-methyl-1,4-oxazepan-6-ol

Atropisomers of rac (6S)-4-(2-(((2R,7aS)-2-fluorotetrahydro-1H-pyrrolizin-7a(5H)-yl)methoxy)-8-(1-(4-methoxybenzyl)-6-methyl-5-(trifluoromethyl)-1H-pyrazolo[3,4-b]pyridin-4-yl)-9-methyl-9H-pyrido[4′,3′:4,5]pyrrolo[2,3-d]pyrimidin-4-yl)-6-methyl-1,4-oxazepan-6-ol (168 mg, 0.213 mmol) were separated by SFC (Column F, 21 to 41% MeCN gradient in H₂O w/0.1% TFA modifier) to give (6S)-4-(2-(((2R,7aS)-2-fluorotetrahydro-1H-pyrrolizin-7a(5H)-yl)methoxy)-8-(1-(4-methoxybenzyl)-6-methyl-5-(trifluoromethyl)-1H-pyrazolo[3,4-b]pyridin-4-yl)-9-methyl-9H-pyrido[4′,3′:4,5]pyrrolo[2,3-d]pyrimidin-4-yl)-6-methyl-1,4-oxazepan-6-ol (the second eluting isomer from SFC). MS (ESI) [M+H]⁺: m/z 790.

### Step C: (6S)-4-(2-(((2R,7aS)-2-fluorotetrahydro-1H-pyrrolizin-7a(5H)-yl)methoxy)-9-methyl-8-(6-methyl-5-(trifluoromethyl)-1H-pyrazolo[3,4-b]pyridin-4-yl)-9H-pyrido[4′,3′:4,5]pyrrolo[2,3-d]pyrimidin-4-yl)-6-methyl-1,4-oxazepan-6-ol (Ex-14)

To (6S)-4-(2-(((2R,7aS)-2-fluorotetrahydro-1H-pyrrolizin-7a(5H)-yl)methoxy)-8-(1-(4-methoxybenzyl)-6-methyl-5-(trifluoromethyl)-1H-pyrazolo[3,4-b]pyridin-4-yl)-9-methyl-9H-pyrido[4′,3′:4,5]pyrrolo[2,3-d]pyrimidin-4-yl)-6-methyl-1,4-oxazepan-6-ol (20 mg, 0.025 mmol) was added TFA (0.2 mL, 2.60 mmol) and trifluoromethanesulfonic acid (0.02 mL, 0.250 mmol) at 20° C. The reaction mixture was stirred at 20° C. for 0.5 h. LCMS showed the starting material was consumed and the desired peak was formed. The reaction mixture was evaporated under reduced pressure to give a crude product. The crude product was purified by preparative HPLC (MeCN/H₂O w/TFA modifier) to give (6S)-4-(2-(((2R,7aS)-2-fluorotetrahydro-1H-pyrrolizin-7a(5H)-yl)methoxy)-9-methyl-8-(6-methyl-5-(trifluoromethyl)-1H-pyrazolo[3,4-b]pyridin-4-yl)-9H-pyrido[4′,3′:4,5]pyrrolo[2,3-d]pyrimidin-4-yl)-6-methyl-1,4-oxazepan-6-ol (Ex-14). MS (ESI) [M+H]⁺: m/z 670. ¹H NMR (400 MHz, MeOD) δ 8.60 (d, J=6.1 Hz, 1H), 8.30 (d, J=6.2 Hz, 1H), 7.74 (s, 1H), 5.51-5.67 (m, 1H), 4.68 (s, 1H), 4.42 (br d, J=13.0 Hz, 1H), 4.31 (br d, J=14.5 Hz, 1H), 4.02-4.08 (m, 2H), 3.83-3.99 (m, 6H), 3.74 (br d, J=12.5 Hz, 1H), 3.43-3.55 (m, 2H), 3.27 (s, 3H), 2.98 (br d, J=1.5 Hz, 3H), 2.57-2.74 (m, 2H), 2.21-2.43 (m, 4H), 1.28 (s, 3H).

### Example 15: (6S)-4-(8-(3,6-dimethyl-5-(trifluoromethyl)-1H-pyrazolo[3,4-b]pyridin-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-15)

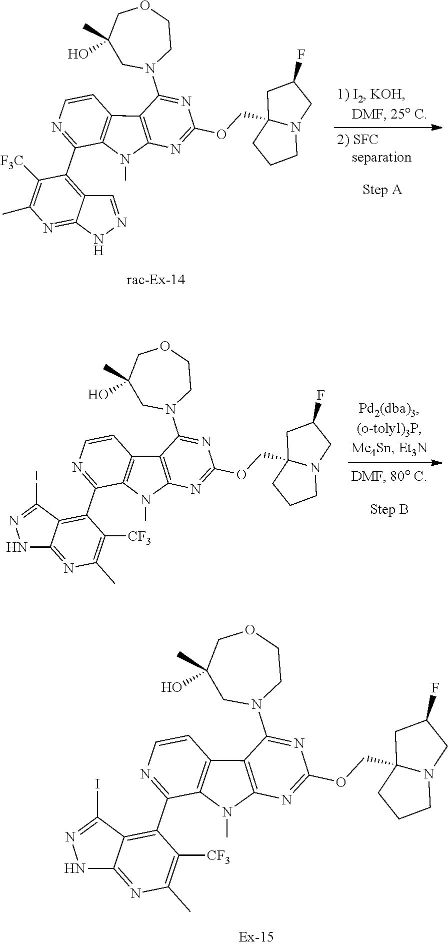 [SMILES: Cc1nc2[nH]ncc2c(-c2nccc3c4c(N5CCOC[C@@](C)(O)C5)nc(OC[C@@]56CCCN5C[C@H](F)C6)nc4n(C)c23)c1C(F)(F)F ; Cc1nc2[nH]nc(I)c2c(-c2nccc3c4c(N5CCOC[C@@](C)(O)C5)nc(OC[C@@]56CCCN5C[C@H](F)C6)nc4n(C)c23)c1C(F)(F)F ; Cc1nc2[nH]nc(I)c2c(-c2nccc3c4c(N5CCOC[C@@](C)(O)C5)nc(OC[C@@]56CCCN5C[C@H](F)C6)nc4n(C)c23)c1C(F)(F)F]

### Step A: (6S)-4-(2-(((2R,7aS)-2-fluorotetrahydro-1H-pyrrolizin-7a(5H)-yl)methoxy)-8-(3-iodo-6-methyl-5-(trifluoromethyl)-1H-pyrazolo[3,4-b]pyridin-4-yl)-9-methyl-9H-pyrido[4′,3′:4,5]pyrrolo[2,3-d]pyrimidin-4-yl)-6-methyl-1,4-oxazepan-6-ol

To a solution of (6S)-4-(2-(((2R,7aS)-2-fluorotetrahydro-1H-pyrrolizin-7a(5H)-yl)methoxy)-9-methyl-8-(6-methyl-5-(trifluoromethyl)-1H-pyrazolo[3,4-b]pyridin-4-yl)-9H-pyrido[4′,3′:4,5]pyrrolo[2,3-d]pyrimidin-4-yl)-6-methyl-1,4-oxazepan-6-ol (rac-Ex-14) (400 mg, 0.597 mmol, mixture of atropisomers obtained from Example 12 in the absence of SFC separation) in DMF (5 mL) was added KOH (134 mg, 2.39 mmol) and 12 (606 mg, 2.39 mmol) at 25° C., and the reaction mixture was stirred at 25° C. for 1 h. LCMS showed the starting material was consumed and the desired MS was found. The reaction mixture was quenched with aq. Na₂SO₃ solution (3 mL) and extracted with EtOAc (30 mL×2). The combined organic phase was washed with brine (10 mL×3), dried over anhydrous Na₂SO₄, filtered and concentrated in vacuo. The residue was purified by silica gel chromatography (petroleum ether:THF=1:2). The atropisomers were separated by preparative SFC (Column G, 40% EtOH w/0.1% NH₃) to afford (6S)-4-(2-(((2R,7aS)-2-fluorotetrahydro-1H-pyrrolizin-7a(5H)-yl)methoxy)-8-(3-iodo-6-methyl-5-(trifluoromethyl)-1H-pyrazolo[3,4-b]pyridin-4-yl)-9-methyl-9H-pyrido[4′,3′:4,5]pyrrolo[2,3-d]pyrimidin-4-yl)-6-methyl-1,4-oxazepan-6-ol (the second eluting isomer from SFC). MS (ESI) [M+H]⁺: m/z 796.

### Step B: 4-(2-(((2R,7aS)-2-fluorotetrahydro-1H-pyrrolizin-7a(5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)-6-methyl-5-(trifluoromethyl)-1H-pyrazolo[3,4-b]pyridine-3-carbonitrile (Ex-15)

To a mixture of (6S)-4-(2-(((2R,7aS)-2-fluorotetrahydro-1H-pyrrolizin-7a(5H)-yl)methoxy)-8-(3-iodo-6-methyl-5-(trifluoromethyl)-1H-pyrazolo[3,4-b]pyridin-4-yl)-9-methyl-9H-pyrido[4′,3′:4,5]pyrrolo[2,3-d]pyrimidin-4-yl)-6-methyl-1,4-oxazepan-6-ol (42 mg, 0.053 mmol) in DMF (1 mL) was added tri-o-tolylphosphine (3.21 mg, 10.6 μmol), Pd₂ (dba): (4.83 mg, 5.28 μmol), tetramethylstannane (28.3 mg, 0.158 mmol) and triethylamine (16.03 mg, 0.158 mmol), and the reaction was stirred at 80° C. for 4 h under nitrogen atmosphere. LCMS showed the staring material disappeared and the desired MS was found. The reaction was diluted with MeCN (1 mL), filtered, and the filtrate was purified by reverse phase preparative HPLC (MeCN/H₂O with NH₄OH modifier) to give (6S)-4-(8-(3,6-dimethyl-5-(trifluoromethyl)-1H-pyrazolo[3,4-b]pyridin-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-15). MS (ESI) [M+H]⁺: m/z 684. ¹H NMR (400 MHz, d⁴-MeOD) δ 8.50 (d, J-5.6 Hz, 1H), 7.99 (d, J=5.6 Hz, 1H), 5.43-5.20 (m, 1H), 4.42-4.34 (m, 1H), 4.34-4.28 (m, 1H), 4.24-4.14 (m, 2H), 4.10-3.98 (m, 2H), 3.97-3.88 (m, 1H), 3.84-3.74 (m, 1H), 3.71 (d, J=12.5 Hz, 1H), 3.50 (d, J=12.5 Hz, 1H), 3.29-3.20 (m, 5H), 3.19-3.15 (m, 1H), 3.02 (dt, J=5.6, 9.4 Hz, 1H), 2.96-2.90 (m, 3H), 2.36-2.17 (m, 2H), 2.16-2.06 (m, 1H), 2.05-1.94 (m, 2H), 1.94-1.80 (m, 1H), 1.49 (s, 3H), 1.26 (s, 3H).

### Example 16:2-amino-7-fluoro-4-(2-(((2R,7aS)-2-fluorotetrahydro-1H-pyrrolizin-7a(5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)benzo[b]thiophene-3-carbonitrile (Ex-16)

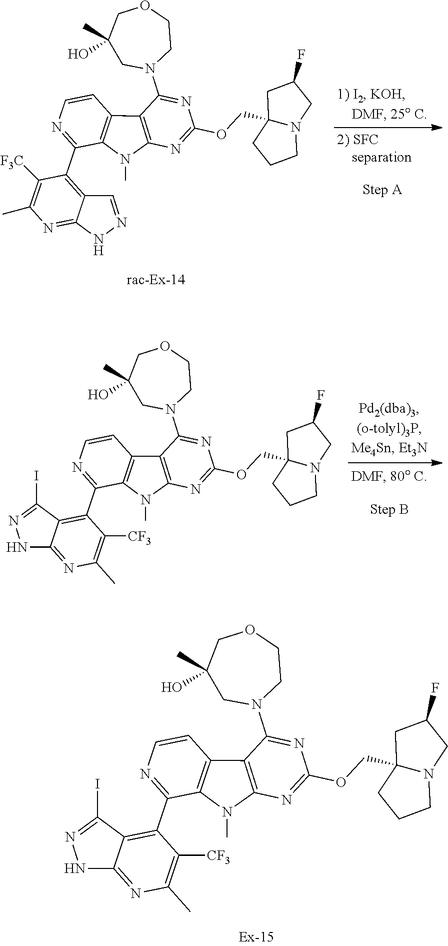 [SMILES: Cc1nc2[nH]ncc2c(-c2nccc3c4c(N5CCOC[C@@](C)(O)C5)nc(OC[C@@]56CCCN5C[C@H](F)C6)nc4n(C)c23)c1C(F)(F)F ; Cc1nc2[nH]nc(I)c2c(-c2nccc3c4c(N5CCOC[C@@](C)(O)C5)nc(OC[C@@]56CCCN5C[C@H](F)C6)nc4n(C)c23)c1C(F)(F)F ; Cc1nc2[nH]nc(I)c2c(-c2nccc3c4c(N5CCOC[C@@](C)(O)C5)nc(OC[C@@]56CCCN5C[C@H](F)C6)nc4n(C)c23)c1C(F)(F)F]

### Step A: Tert-butyl(3-cyano-7-fluoro-4-(2-(((2R,7aS)-2-fluorotetrahydro-1H-pyrrolizin-7a (5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)benzo[b]thiophen-2-yl) carbamate

To a solution of(S)-4-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Int-137) (150 mg, 0.297 mmol) (divided into 3 batches, 50 mg each) in toluene (16 mL) was added tert-butyl(3-cyano-4-(5,5-dimethyl-1,3,2-dioxaborinan-2-yl)-7-fluorobenzo[b]thiophen-2-yl) carbamate (192 mg, 0.475 mmol), sodium tert-butoxide (86 mg, 0.891 mmol), palladium (II) acetate (33.3 mg, 0.149 mmol), and rac-3-(tert-butyl)-4-(2,6-dimethoxyphenyl)-2,3-dihydrobenzo[d][1,3]oxaphosphole (rac-BI-DIME) (98 mg, 0.297 mmol) in a glovebox. The reaction was stirred at 80° C. for 16 h under N₂ atmosphere. LCMS showed the starting material was consumed and a major peak with the desired MS was observed. The reaction mixture was cooled to r.t., diluted with EtOAc (30 mL), washed with brine (5 mL), and the organic layer was dried and concentrated in vacuo. The residue was purified by flash silica gel chromatography (50% THF in petroleum ether) to give tert-butyl(3-cyano-7-fluoro-4-(2-(((2R,7aS)-2-fluorotetrahydro-1H-pyrrolizin-7a(5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)benzo[b]thiophen-2-yl) carbamate. MS (ESI) [M+H]⁺: m/z 761.

### Step B: 2-amino-7-fluoro-4-(2-(((2R,7aS)-2-fluorotetrahydro-1H-pyrrolizin-7a (5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)benzo[b]thiophene-3-carbonitrile (Ex-16)

A solution of tert-butyl(3-cyano-7-fluoro-4-(2-(((2R,7aS)-2-fluorotetrahydro-1H-pyrrolizin-7a(5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)benzo[b]thiophen-2-yl) carbamate (70 mg, 0.092 mmol) in DCM (1 mL) and TFA (0.5 mL) was stirred at 25° C. for 1 h. LCMS showed the reaction was complete. The reaction was concentrated in vacuo and the residue was diluted with EtOAc (5 mL), basified with sat. aq. NaHCO₃ (1 mL), and the organic layer was concentrated in vacuum. The residue was purified by reverse-phase preparative HPLC (MeCN/H₂O with NH₄OH modifier) to give 2-amino-7-fluoro-4-(2-(((2R,7aS)-2-fluorotetrahydro-1H-pyrrolizin-7a(5H)-yl)methoxy)-4-((S)-6-hydroxy-6-methyl-1,4-oxazepan-4-yl)-9-methyl-9H-pyrido[4′,3′:4,5]pyrrolo[2,3-d]pyrimidin-8-yl)benzo[b]thiophene-3-carbonitrile (Ex-16). MS (ESI) [M+H]⁺: m/z 661. ¹H NMR (400 MHz, CDCl₃) δ 8.52 (dd, J=5.36, 9.30 Hz, 1H), 7.61-7.68 (m, 1H), 7.30-7.47 (m, 1H), 7.00-7.06 (m, 1H), 6.86-6.99 (m, 1H), 5.90-6.08 (m, 2H), 5.15-5.36 (m, 1H), 4.46 (br d, J=13.47 Hz, 1H), 4.22-4.31 (m, 1H), 4.13-4.20 (m, 1H), 4.04-4.12 (m, 1H), 3.90-4.02 (m, 1H), 3.77 (brt, J=11.98 Hz, 2H), 3.55 (br dd, J=4.41, 15.02 Hz, 1H), 3.45 (br d, J=11.21 Hz, 1H), 3.34 (br d, J=12.52 Hz, 1H), 3.27 (br d, J=19.91 Hz, 4H), 3.18-3.23 (m, 1H), 3.08-3.17 (m, 1H), 2.96 (br d, J=5.13 Hz, 1H), 2.15-2.32 (m, 2H), 2.04-2.14 (m, 2H), 1.78-1.93 (m, 2H), 1.36 (br d, J=3.34 Hz, 3H).

### Example 17: (S)-4-(8-(3-amino-8-ethynyl-7-fluoroisoquinolin-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-17)

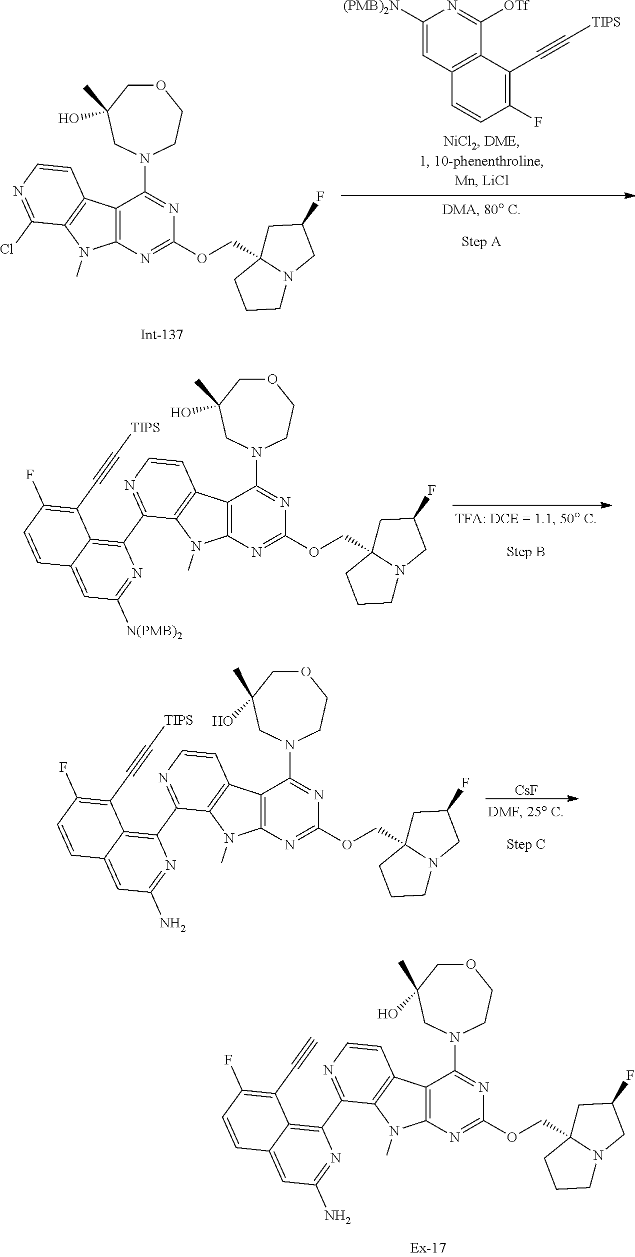

### Step A: (S)-4-(8-(3-(bis(4-methoxybenzyl)amino)-7-fluoro-8-((triisopropylsilyl)ethynyl) isoquinolin-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol

To a solution of 1,10-phenanthroline (17.1 mg, 0.095 mmol) in DMA (1 mL) was added nickel (II) chloride ethylene glycol dimethyl ether complex (17.4 mg, 0.079 mmol) in a glovebox, and the mixture was stirred at 50° C. for 1 h. Manganese dust (17.4 mg, 0.317 mmol), (S)-4-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Int-137) (40 mg, 0.079 mmol), lithium chloride (5.04 mg, 0.119 mmol), and 3-(bis(4-methoxybenzyl)amino)-7-fluoro-8-((triisopropylsilyl)ethynyl) isoquinolin-1-yl trifluoromethanesulfonate (87 mg, 0.119 mmol) were added to the mixture. Then the reaction was stirred at 80° C. for another 3 h. LCMS showed the starting material was consumed and the desired mass was found. The reaction mixture was diluted with EtOAc (15 mL) and washed with brine (4×2 mL). The organic layer was dried over sodium sulfate, filtered, and concentrated in vacuo. The residue was purified by preparative TLC (SiO₂, DCM:MeOH=15:1) to give(S)-4-(8-(3-(bis(4-methoxybenzyl)amino)-7-fluoro-8-((triisopropylsilyl)ethynyl) isoquinolin-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol. MS (ESI) [M/2+H]⁺: m/z 526.

### Step B: (S)-4-(8-(3-amino-7-fluoro-8-((triisopropylsilyl)ethynyl) isoquinolin-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepam-6-ol

To a solution of(S)-4-(8-(3-(bis(4-methoxybenzyl)amino)-7-fluoro-8-((triisopropylsilyl)ethynyl) isoquinolin-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (80 mg, 0.076 mmol) in DCE (0.5 mL) was added TFA (0.5 mL), then the mixture was stirred at 50° C. for 1 h. LCMS showed the starting material was consumed and the desired mass was found. The reaction was concentrated in vacuo to afford(S)-4-(8-(3-amino-7-fluoro-8-((triisopropylsilyl)ethynyl) isoquinolin-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol. MS (ESI) [M+H]⁺: m/z 811.

### Step C: (S)-4-(8-(3-amino-8-ethynyl-7-fluoroisoquinolin-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepam-6-ol (Ex-17)

To a solution of(S)-4-(8-(3-amino-7-fluoro-8-((triisopropylsilyl)ethynyl) isoquinolin-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (61.7 mg, 0.076 mmol) in DMF (1 mL) was added CsF (57.8 mg, 0.380 mmol), the reaction was stirred at 25° C. for 3 h. LCMS showed the reaction was complete. The reaction mixture was concentrated in vacuo and the residue was purified by preparative reverse-phase HPLC (MeCN/H₂O with NH₄OH modifier) to give(S)-4-(8-(3-amino-8-ethynyl-7-fluoroisoquinolin-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-17). MS (ESI) [M+H]⁺: m/z 655. ¹H NMR (400 MHz, MeOD) δ8.37 (d, J=5.48 Hz, 1H), 7.91 (d, J=5.48 Hz, 1H), 7.76 (dd, J=5.42, 9.24 Hz, 1H), 7.41 (t, J=9.00 Hz, 1H), 7.06 (s, 1H), 5.29 (d, J=54.0 Hz, 1H), 4.34-4.44 (m, 1H), 4.25-4.33 (m, 1H), 4.10-4.22 (m, 2H), 3.91-4.07 (m, 2H), 3.81-3.89 (m, 1H), 3.57-3.79 (m, 2H), 3.37-3.50 (m, 1H), 3.22-3.29 (m, 3H), 3.12-3.22 (m, 4H), 2.94-3.01 (m, 1H), 2.14-2.35 (m, 2H), 2.04-2.12 (m, 1H), 1.91-2.02 (m, 2H), 1.76-1.89 (m, 1H), 1.25 (d, J=6.20 Hz, 3H).

### Example 18: (S)-4-(8-(8-ethynyl-7-fluoro-3-hydroxynaphthalen-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-18)

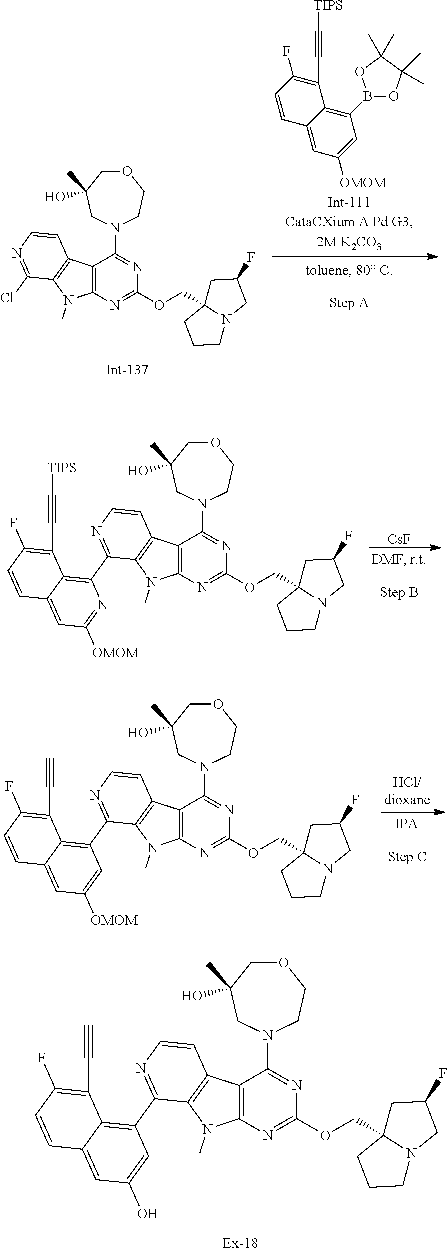

### Step A: (S)-4-(8-(7-fluoro-3-(methoxymethoxy)-8-((triisopropylsilyl)ethynyl) naphthalen-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol

To a solution of(S)-4-(8-chloro-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Int-137) (600 mg, 1.19 mmol) (divided into 12 batches, 50 mg each) in toluene (48 mL) was added ((2-fluoro-6-(methoxymethoxy)-8-(4,4,5,5-tetramethyl-1,3,2-dioxaborolan-2-yl) naphthalen-1-yl) ethynyl)triisopropylsilane (Int-112) (974 mg, 1.90 mmol), K₂CO₃ (1.78 mL, 3.56 mmol, 2 M in H₂O) and CataCXium A Pd G3 (433 mg, 0.594 mmol) in a glovebox. The reactions were stirred at 80° C. for 16 h under N₂ atmosphere. LCMS showed the reactions were complete and a major peak with the desired MS was observed. The reactions were cooled to r.t., combined, and diluted with EtOAc (60 mL). The reactions were washed with brine (10 mL). The organic layer was dried over anhydrous Na₂SO₄, filtered, and concentrated in vacuo. The residue was purified by flash silica gel chromatography (0-50% THF in petroleum ether) to give(S)-4-(8-(7-fluoro-3-(methoxymethoxy)-8-((triisopropylsilyl)ethynyl) naphthalen-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol. MS (ESI) [M+H]⁺: m/z 855.

### Step B: (S)-4-(8-(8-ethynyl-7-fluoro-3-(methoxymethoxy) naphthalen-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol

To a solution of(S)-4-(8-(7-fluoro-3-(methoxymethoxy)-8-((triisopropylsilyl)ethynyl) naphthalen-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (200 mg, 0.234 mmol) in DMF (2 mL) was added CsF (178 mg, 1.17 mmol). The reaction was stirred at r.t. for 1 h. LCMS showed the reaction was finished and a major peak with the desired MS was observed. The reaction mixture was diluted with EtOAc (30 mL), washed with brine (2×5 mL), and the organic layer was concentrated in vacuo to afford(S)-4-(8-(8-ethynyl-7-fluoro-3-(methoxymethoxy) naphthalen-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol, which was used directly in the next step without further purification. MS (ESI) [M+H]⁺: m/z 699.

### Step C: (S)-4-(8-(8-ethynyl-7-fluoro-3-hydroxynaphthalen-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-18)

A solution of(S)-4-(8-(8-ethynyl-7-fluoro-3-(methoxymethoxy) naphthalen-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (160 mg, 0.229 mmol) in i-PrOH (2 mL) and HCl (4 M in dioxane, 2 mL) was stirred at 25° C. for 1 h. LCMS showed the reaction was complete. The reaction was concentrated in vacuo and the residue was diluted with EtOAc (10 mL), washed with sat. aq. NaHCO₃ (2 mL), and brine (2 mL). The organic layer was concentrated in vacuo. The residue was purified by preparative reverse-phase HPLC (MeCN/H₂O with NH₄OH modifier) to give(S)-4-(8-(8-ethynyl-7-fluoro-3-hydroxynaphthalen-1-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-18). MS (ESI) [M+H]⁺: m/z 655. ¹H NMR (400 MHz, MeOD) δ 8.37-8.31 (m, 1H), 7.91-7.83 (m, 2H), 7.40 (s, 1H), 7.32 (t, J=8.94 Hz, 1H), 7.27-7.20 (m, 1H), 5.37-5.17 (m, 1H), 4.39 (br s, 1H), 4.27 (d, J=10.37 Hz, 1H), 4.20-4.09 (m, 2H), 3.98 (brt, J=13.11 Hz, 2H), 3.80 (d, J=14.90 Hz, 1H), 3.69 (d, J=12.52 Hz, 1H), 3.63-3.55 (m, 1H), 3.51-3.37 (m, 1H), 3.28-3.19 (m, 2H), 3.18-3.13 (m, 3H), 3.09-3.06 (m, 1H), 3.04-2.90 (m, 2H), 2.32-2.11 (m, 2H), 2.11-2.01 (m, 1H), 2.00-1.91 (m, 2H), 1.90-1.79 (m, 1H), 1.31-1.22 (m, 3H).

### Example 19: (6S)-4-(8-(5-(difluoromethyl)-6-methyl-1H-indazol-4-yl)-2-(((R)-1-((dimethylamino)methyl)-2,2-difluorocyclopropyl)methoxy)-9-methyl-9H-pyrido[4′,3′:4,5]pyrrolo[2,3-d]pyrimidin-4-yl)-6-methyl-1,4-oxazepan-6-ol (Ex-19)

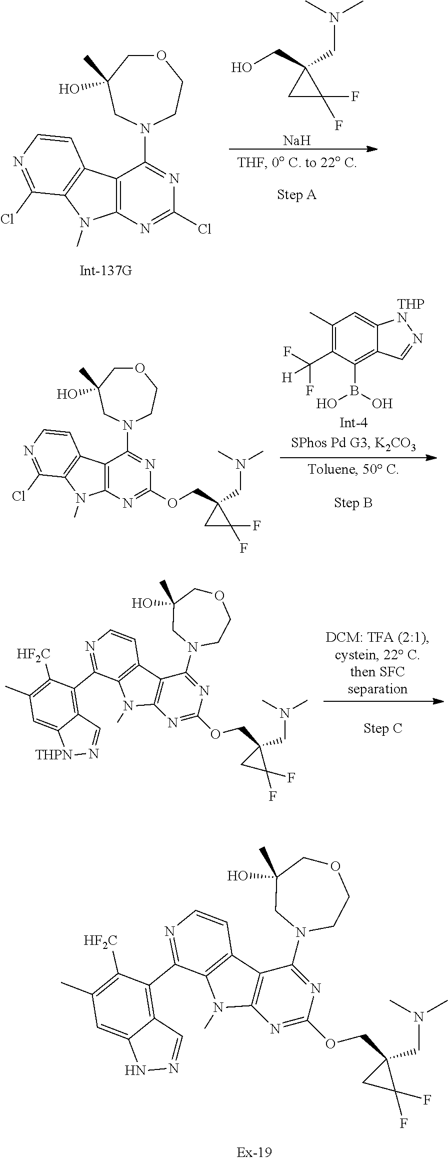

### Step A: (S)-4-(8-chloro-2-(((R)-1-((dimethylamino)methyl)-2,2-difluorocyclopropyl)methoxy)-9-methyl-9H-pyrido[4′,3′:4,5]pyrrolo[2,3-d]pyrimidin-4-yl)-6-methyl-1,4-oxazepan-6-ol

A vial was loaded with (R)-(1-((dimethylamino)methyl)-2,2-difluorocyclopropyl) methanol (472 mg, 2.86 mmol) and THF (3 mL). The mixture was cooled to 0° C. and NaH (190 mg, 4.76 mmol) was added. The reaction was stirred at room temperature for 1 hour. The mixture was added over a solution of(S)-4-(2,8-dichloro-9-methyl-9H-pyrido[4′,3′:4,5]pyrrolo[2,3-d]pyrimidin-4-yl)-6-methyl-1,4-oxazepan-6-ol (Int-137G) (728 mg, 1.90 mmol) in THF (10 mL). The reaction was warmed to r.t. and stirred for 1.5 hours. LCMS showed full conversion of starting material to product. The mixture was quenched with sat. aq. NH₄Cl, extracted with EtOAc (3×25 mL) and washed with brine (2×10 mL). The combined organic phase was dried over Na₂SO₄, filtered, and the filtrate was evaporated under reduced pressure to afford a residue, which was purified by silica gel chromatography (30% MeOH in DCM) to obtain(S)-4-(8-chloro-2-(((R)-1-((dimethylamino)methyl)-2,2-difluorocyclopropyl)methoxy)-9-methyl-9H-pyrido[4′,3′:4,5]pyrrolo[2,3-d]pyrimidin-4-yl)-6-methyl-1,4-oxazepan-6-ol. MS (ESI): m/z (M+H)⁺ 511.

### Step B: (6S)-4-(8-(5-(difluoromethyl)-6-methyl-1-(tetrahydro-2H-pyran-2-yl)-1H-indazol-4-yl)-2-(((R)-1-((dimethylamino)methyl)-2,2-difluorocyclopropyl)methoxy)-9-methyl-9H-pyrido[4′,3′:4,5]pyrrolo[2,3-d]pyrimidin-4-yl)-6-methyl-1,4-oxazepan-6-ol

A vial was loaded with (S)-4-(8-chloro-2-(((R)-1-((dimethylamino)methyl)-2,2-difluorocyclopropyl)methoxy)-9-methyl-9H-pyrido[4′,3′:4,5]pyrrolo[2,3-d]pyrimidin-4-yl)-6-methyl-1,4-oxazepan-6-ol (74.5 mg, 0.146 mmol) and SPhos Pd G3 (57.9 mg, 0.073 mmol) and the vial was placed under nitrogen. Toluene (0.5 mL) was added via a syringe, then degassed K₂CO₃ (115 μL, 0.230 mmol, 2 M in H₂O) was added via a syringe. The mixture was allowed to stir for 5 minutes at r.t. After this time, a solution of (5-(difluoromethyl)-6-methyl-1-(tetrahydro-2H-pyran-2-yl)-1H-indazol-4-yl)boronic acid (Int-4) (54 mg, 0.18 mmol) in 0.5 mL toluene was added via a syringe. The mixture was heated at 50° C. for 16 hours, and then at 90° C. for two hours. The reaction was quenched with water (2 mL) and extracted with DCM (3×10 mL). The organic layer was dried over Na₂SO₄, filtered, and evaporated under reduced pressure to afford crude (6S)-4-(8-(5-(difluoromethyl)-6-methyl-1-(tetrahydro-2H-pyran-2-yl)-1H-indazol-4-yl)-2-(((R)-1-((dimethylamino)methyl)-2,2-difluorocyclopropyl)methoxy)-9-methyl-9H-pyrido[4′,3′:4,5]pyrrolo[2,3-d]pyrimidin-4-yl)-6-methyl-1,4-oxazepan-6-ol, which was used directly in the next step without further purification. MS (ESI): m/z (M+H)⁺ 741.

### Step C: (6S)-4-(8-(5-(difluoromethyl)-6-methyl-1H-indazol-4-yl)-2-(((R)-1-((dimethylamino)methyl)-2,2-difluorocyclopropyl)methoxy)-9-methyl-9H-pyrido[4′,3′:4,5]pyrrolo[2,3-d]pyrimidin-4-yl)-6-methyl-1,4-oxazepan-6-ol (Ex-19)

A vial was loaded with (6S)-4-(8-(5-(difluoromethyl)-6-methyl-1-(tetrahydro-2H-pyran-2-yl)-1H-indazol-4-yl)-2-(((R)-1-((dimethylamino)methyl)-2,2-difluorocyclopropyl)methoxy)-9-methyl-9H-pyrido[4′,3′:4,5]pyrrolo[2,3-d]pyrimidin-4-yl)-6-methyl-1,4-oxazepan-6-ol (108 mg, 0.146 mmol) and cysteine (18 mg, 0.15 mmol) and the solids were dissolved in DCM (1.5 mL) and TFA (0.8 mL). The solution was stirred at r.t. for 30 minutes. After this time, LCMS showed full conversion of the starting material. The volatiles were evaporated, and the crude product was redissolved in DMSO, filtered and subjected to preparative reverse-phase HPLC purification (MeCN/H₂O with TFA modifier). The atropisomers were separated by SFC purification (Column D, 35% MeOH with 0.1% NH₄OH) to afford (6S)-4-(8-(5-(difluoromethyl)-6-methyl-1H-indazol-4-yl)-2-(((R)-1-((dimethylamino)methyl)-2,2-difluorocyclopropyl)methoxy)-9-methyl-9H-pyrido[4′,3′:4,5]pyrrolo[2,3-d]pyrimidin-4-yl)-6-methyl-1,4-oxazepan-6-ol (Ex-19) (the first eluting isomer from SFC). MS (ESI): m/z (M+H)⁺ 657. ¹H NMR (600 MHz, MeOD) δ 8.51 (d, J=5.5 Hz, 1H), 7.97 (d, J=5.6 Hz, 1H), 7.69 (s, 1H), 7.50 (s, 1H), 6.56 (t, J=53.7 Hz, 1H), 4.67-4.48 (m, 2H), 4.43 (d, J=13.9 Hz, 1H), 4.24 (d, J=14.7 Hz, 1H), 4.11-3.97 (m, 2H), 3.91 (d, J=14.7 Hz, 1H), 3.86-3.77 (m, 1H), 3.73 (d, J=12.5 Hz, 1H), 3.50 (d, J=12.5 Hz, 1H), 3.13 (s, 3H), 2.85 (d, J=13.0 Hz, 1H), 2.78 (s, 3H), 2.45 (d, J=13.0 Hz, 1H), 2.29 (s, 6H), 1.69-1.58 (m, 1H), 1.43-1.36 (m, 1H), 1.33-1.23 (m, 4H).

The example in the table below was synthesized using a similar procedure as described in the synthesis of Ex-19 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | SFC | | |
|---|---|---|---|---|---|
| | Starting | | Separation | | [M + H]⁺ |
| Ex. | Material | Structure | Conditions | Compound Name | Found |
| Ex- 20 | 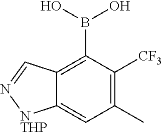 Int-1 | 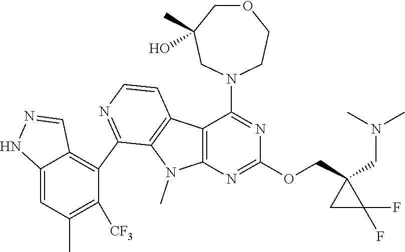 [SMILES: Cc1cc2[nH]ncc2c(-c2nccc3c4c(N5CCOC[C@@](C)(O)C5)nc(OC[C@]5(CN(C)C)CC5(F)F)nc4n(C)c23)c1C(F)(F)F] | Column C, 30% MeOH w/0.1% NH₄OH | (6S)-4-(2-(((R)-1- ((dimethylamino)meth- yl)-2,2-difluoro- cyclopropyl)meth- oxy)-9-methyl-8- (6-methyl-5- (trifluoromethyl)-1H- indazol-4-yl)-9H- pyrido[4′,3′:4,5]pyrrolo [2,3-d]pyrimidin-4- yl)-6-methyl-1,4- oxazepan-6-ol | 675 |

### Example 21:6-(2-(((2R,7aS)-2-fluorotetrahydro-1H-pyrrolizin-7a(5H)-yl)methoxy)-9-methyl-8-(6-methyl-5-(trifluoromethyl)-1H-indazol-4-yl)-9H-pyrido[4′,3′:4,5]pyrrolo[2,3-d]pyrimidin-4-yl)-1,6-diazaspiro[3.5]nonan-2-one (Ex-21)

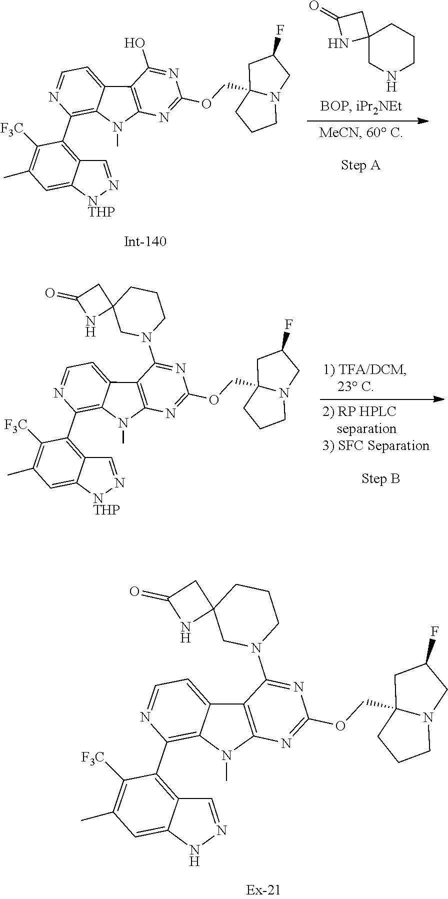

### Step A: 6-(2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-1,6-diazaspiro[3.5]nonan-2-one

2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-ol (Int-140) (30 mg, 0.047 mmol), rac-1,6-diazaspiro[3.5]nonan-2-one (13 mg, 0.094 mmol), and BOP (42 mg, 0.094 mmol) were added to a vial with a stir bar. Acetonitrile (469 μL) and DIPEA (49 μL, 0.28 mmol) were added via a syringe. The vial was fitted with a septum and heated to 60° C. for 1 h. After 1 h, reaction was complete by LCMS. The reaction was cooled to r.t., diluted with water and EtOAc (5 mL each), the layers were shaken, and separated. The aqueous phase was extracted with EtOAc (2×5 mL). The combined organics were dried over sodium sulfate, filtered, and concentrated to provide 6-(2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-1,6-diazaspiro[3.5]nonan-2-one which was used in the next step without further purification. MS (ESI): m/z (M+H)⁺ 762.

### Step B: 6-(2-(((2R,7aS)-2-fluorotetrahydro-1H-pyrrolizin-7a(5H)-yl)methoxy)-9-methyl-8-(6-methyl-5-(trifluoromethyl)-1H-indazol-4-yl)-9H-pyrido[4′,3′:4,5]pyrrolo[2,3-d]pyrimidin-4-yl)-1,6-diazaspiro[3.5]nonan-2-one (Ex-21)

The crude reaction mixture including 6-(2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-1,6-diazaspiro[3.5]nonan-2-one (36 mg, 0.047 mmol) was added to a vial with stir bar, dissolved in DCM (1 mL) and TFA (0.3 mL), and allowed to stir for 1 h. After 1 h, the reaction was complete by LCMS. The reaction was diluted with DCM (5 mL) and quenched with sat. aq. NaHCO₃ (5 mL). The layers were shaken and separated and the aqueous phase was washed with DCM (2×5 mL). The combined organics were washed with brine (10 mL), dried over sodium sulfate, filtered, and concentrated. The crude material was purified and the product diastereomers were separated by preparative reverse-phase HPLC (MeCN/H₂O w/TFA modifier). The resulting enantiomers were separated by SFC (Column H, 50% MeOH w/0.1% NH₄OH) to obtain 6-(2-(((2R,7aS)-2-fluorotetrahydro-1H-pyrrolizin-7a(5H)-yl)methoxy)-9-methyl-8-(6-methyl-5-(trifluoromethyl)-1H-indazol-4-yl)-9H-pyrido[4′,3′:4,5]pyrrolo[2,3-d]pyrimidin-4-yl)-1,6-diazaspiro[3.5]nonan-2-one (Ex-21) (the first eluting isomer from SFC). MS (ESI): m/z (M+H)⁺ 678. ¹H NMR (500 MHz, d⁴-MeOH) δ 8.47 (d, J=5.4 Hz, 1H), 7.89 (d, J=5.3 Hz, 1H), 7.78 (s, 1H), 7.38 (s, 1H), 5.34 (d, J=53.5 Hz, 1H), 4.36 (d, J=10.7 Hz, 1H), 4.25 (d, J=10.8 Hz, 1H), 4.18 (d, J=13.0 Hz, 1H), 4.05 (m, 1H), 3.94 (d, J=13.3 Hz, 1H), 3.79 (m, 1H), 3.14 (s, 3H), 3.09 (m, 1H), 2.92-2.88 (m, 1H), 2.80 (m, 4H), 2.42-1.91 (m, 13H).

The examples in the table below were synthesized using a similar procedure as described in the synthesis of Ex-21 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | SFC | | |
|---|---|---|---|---|---|
| | Starting | | Separation | | [M + H]⁺ |
| Ex. | Material | Structure | Conditions | Compound Name | Found |
| Ex- 20 | 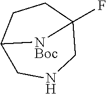 [SMILES: CC(C)(C)OC(=O)N1C2CCC1(F)CNC2] Int-36 | 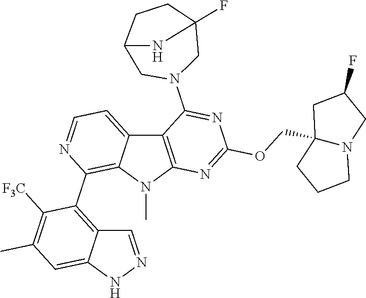 [SMILES: Cc1cc2[nH]ncc2c(-c2nccc3c4c(N5CC6CCC(F)(C5)N6)nc(OC[C@@]56CCCN5C[C@H](F)C6)nc4n(C)c23)c1C(F)(F)F] | Column H, 50% MeOH w/0.1% NH₄OH, first eluting peak | 4-(1-fluoro-3,8- diazabicyclo[3.2.1]octan- 3-yl)-2-(((2R,7aS)-2- fluorotetrahydro-1H- pyrrolizin-7a(5H)- yl)methoxy)-9-methyl- 8-(6-methyl-5- (trifluoromethyl)-1H- indazol-4-yl)-9H- pyrido[4′,3′:4,5]pyrrolo[2,3- d]pyrimidine | 668 |
| Ex- 21 | 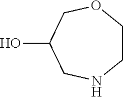 | 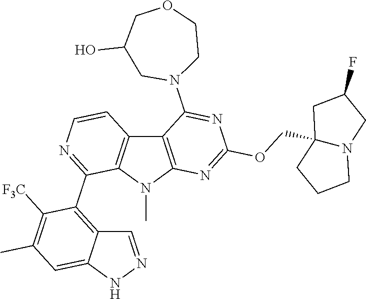 [SMILES: Cc1cc2[nH]ncc2c(-c2nccc3c4c(N5CCOCC(O)C5)nc(OC[C@@]56CCCN5C[C@H](F)C6)nc4n(C)c23)c1C(F)(F)F] | Column C, 45% MeOH w/0.1% NH₄OH, 70 mL/min, third eluting peak | 4-(2-(((2R,7aS)-2- fluorotetrahydro-1H- pyrrolizin-7a(5H)- yl)methoxy)-9-methyl- 8-(6-methyl-5- (trifluoromethyl)-1H- indazol-4-yl)-9H- pyrido[4′,3′:4,5]pyrrolo[2,3- d]pyrimidin-4-yl)- 1,4-oxazepan-6-ol | 655 |

### Example 24: (6S)-4-(9-ethyl-2-(((2R,7aS)-2-fluorotetrahydro-1H-pyrrolizin-7a(5H)-yl)methoxy)-8-(6-methyl-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol (Ex-24)

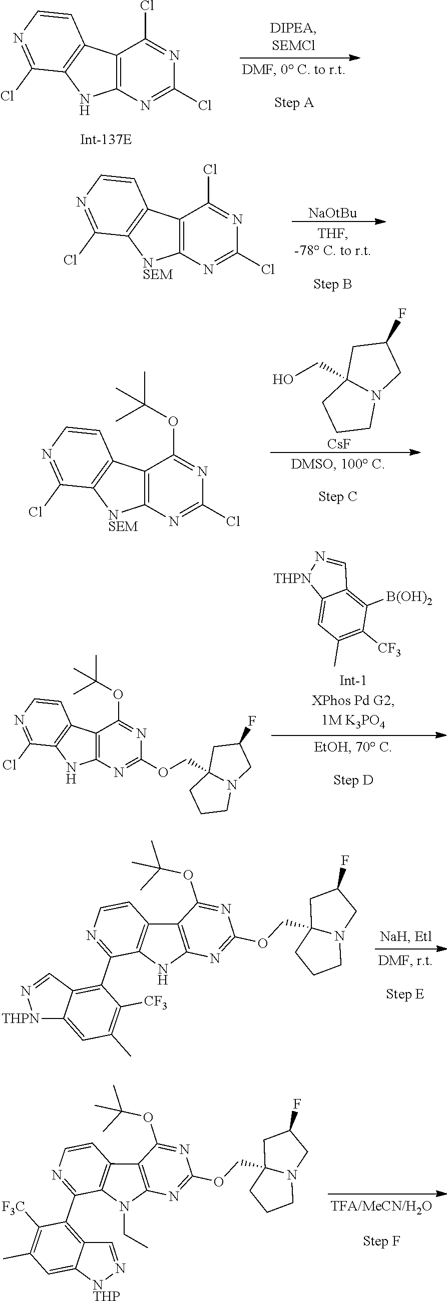

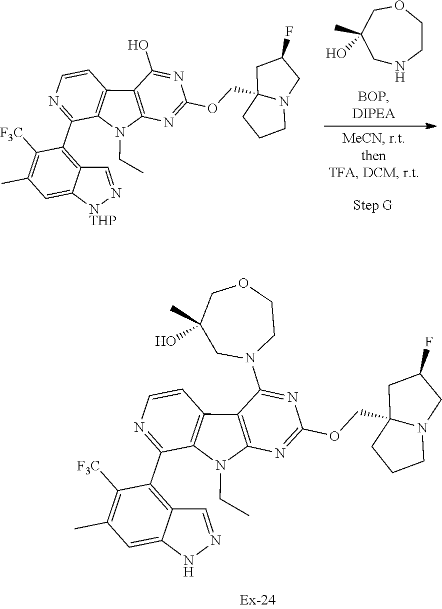

### Step A: 2,4,8-trichloro-9-((2-(trimethylsilyl)ethoxy)methyl)-9H-pyrido[4′,3′:4,5]pyrrolo[2,3-d]pyrimidine

N,N-Diisopropylethylamine (2.5 mL, 15 mmol) followed immediately by (2-(chloromethoxy)ethyl)trimethylsilane (1.9 mL, 11 mmol) were added to a solution of 2,4,8-trichloro-9H-pyrido[4′,3′:4,5]pyrrolo[2,3-d]pyrimidine (Int-137E) (2.00 g, 7.31 mmol) in DMF (24 mL) at 0° C. The reaction mixture was allowed to warm to r.t. and stirred for 4 h. After 4 h, the reaction mixture was concentrated in vacuo. The product was purified by silica gel chromatography (0 to 60% ethyl acetate in hexane) to provide 2,4,8-trichloro-9-((2-(trimethylsilyl)ethoxy)methyl)-9H-pyrido[4′,3′:4,5]pyrrolo[2,3-d]pyrimidine. MS (ESI) [M+H]⁺ m/z 403, 405.

### Step B: 4-(tert-butoxy)-2,8-dichloro-9-((2-(trimethylsilyl)ethoxy)methyl)-9H-pyrido[4′,3′:4,5]pyrrolo[2,3-d]pyrimidine

Sodium tert-butoxide (3.5 ml, 7.0 mmol, 2M in THF) was added to a solution of 2,4,8-trichloro-9-((2-(trimethylsilyl)ethoxy)methyl)-9H-pyrido[4′,3′:4,5]pyrrolo[2,3-d]pyrimidine (2.6 g, 6.4 mmol) in THF (64.3 ml) at −78° C. The reaction mixture was allowed to warm to r.t. and stirred overnight. The reaction mixture was concentrated in vacuo and purified by silica gel chromatography (0 to 60% ethyl acetate in hexane) to provide 4-(tert-butoxy)-2,8-dichloro-9-((2-(trimethylsilyl)ethoxy)methyl)-9H-pyrido[4′,3′:4,5]pyrrolo[2,3-d]pyrimidine. MS (ESI) [M+H]⁺ m/z 441, 443.

### Step C: 4-(tert-butoxy)-8-chloro-2-(((2R,7aS)-2-fluorotetrahydro-1H-pyrrolizin-7a(5H)-yl)methoxy)-9H-pyrido[4′,3′:4,5]pyrrolo[2,3-d]pyrimidine

Cesium fluoride (1.6 g, 11 mmol) was added to a solution of 4-(tert-butoxy)-2,8-dichloro-9-((2-(trimethylsilyl)ethoxy)methyl)-9H-pyrido[4′,3′:4,5]pyrrolo[2,3-d]pyrimidine (800 mg, 1.81 mmol) and ((2R,7aS)-2-fluorotetrahydro-1H-pyrrolizin-7a(5H)-yl) methanol (577 mg, 3.62 mmol) in DMSO (9.0 ml). The reaction mixture was stirred at 100° C. overnight. The reaction mixture was concentrated in vacuo. The product was purified by silica gel chromatography (0 to 100% (3:1 EtOH:EtOAc) in hexane with 0.1% Et₃N additive) to provide 4-(tert-butoxy)-8-chloro-2-(((2R,7aS)-2-fluorotetrahydro-1H-pyrrolizin-7a(5H)-yl)methoxy)-9H-pyrido[4′,3′:4,5]pyrrolo[2,3-d]pyrimidine. MS (ESI) [M+H]⁺ m/z 434.

### Step D: 4-(tert-butoxy)-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidine

A mixture of 4-(tert-butoxy)-8-chloro-2-(((2R,7aS)-2-fluorotetrahydro-1H-pyrrolizin-7a(5H)-yl)methoxy)-9H-pyrido[4′,3′:4,5]pyrrolo[2,3-d]pyrimidine (190 mg, 0.436 mmol), XPhos Pd G2 (69 mg, 0.087 mmol), and (6-methyl-1-(tetrahydro-2H-pyran-2-yl)-5-(trifluoromethyl)-1H-indazol-4-yl)boronic acid (Int-1) (286 mg, 0.871 mmol) was sealed in a vial and purged with argon. Ethanol (3.0 mL) and potassium phosphate tribasic (870 μl, 0.870 mmol, 1M in H₂O) was added to the reaction mixture and the reaction mixture was stirred at 70° C. overnight. The reaction mixture was filtered and concentrated in vacuo. The product was purified by silica gel chromatography (0 to 100% EtOAc in hexane then 0 to 100% (3:1 EtOH:EtOAc) in EtOAc) to provide 4-(tert-butoxy)-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidine. MS (ESI) [M+H]⁺ m/z 682.

### Step E: 4-(tert-butoxy)-9-ethyl-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidine

Sodium hydride (13 mg, 0.32 mmol, 60 wt % in mineral oil) was added to a solution of 4-(tert-butoxy)-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidine (110 mg, 0.160 mmol) in DMF (1600 μl). The reaction mixture was stirred for 10 min prior to the addition of ethyl iodide (26 μl, 0.32 mmol) to the reaction mixture. The reaction mixture was allowed to stir overnight at r.t. The reaction mixture was concentrated in vacuo and purified by silica gel chromatography (0 to 100% (3:1 EtOH:EtOAc) in hexane with 0.1% Et₃N additive) to provide 4-(tert-butoxy)-9-ethyl-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidine. MS (ESI) [M+H-t-Bu]⁺ m/z 654.

### Step F: 9-ethyl-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-ol

TFA (28 μl) was added to a solution of 4-(tert-butoxy)-9-ethyl-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidine (98 mg, 0.14 mmol) in acetonitrile (1.08 ml) and water (0.28 ml). The reaction mixture was stirred overnight at r.t. The reaction mixture was concentrated in vacuo and the resulting 9-ethyl-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-ol was used directly in the next step without further purification. MS (ESI) [M+H]⁺ m/z 654.

### Step G: (6S)-4-(9-ethyl-2-(((2R,7aS)-2-fluorotetrahydro-1H-pyrrolizin-7a(5H)-yl)methoxy)-8-(6-methyl-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol (Ex-24)

N,N-Diisopropylethylamine (60 μl, 0.34 mmol) followed by BOP (46 mg, 0.10 mmol) was added to a solution of 9-ethyl-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-ol (45 mg, 0.069 mmol) in acetonitrile (344 μl). The reaction mixture was stirred for 30 min before adding it to neat(S)-6-methyl-1,4-oxazepan-6-ol (18 mg, 0.14 mmol). The reaction mixture was stirred overnight. The reaction mixture was concentrated in vacuo and redissolved in DCM (344 μl) and TFA (344 μl) and allowed to stir for 4 h. The reaction mixture was concentrated in vacuo, redissolved in DMSO (1 mL), and treated with aq. ammonium hydroxide (300 μL). The reaction mixture was filtered to remove precipitate and the product was purified by preparative reverse-phase HPLC (MeCN/H₂O with 0.05% NH₄OH additive) to provide (6S)-4-(9-ethyl-2-(((2R,7aS)-2-fluorotetrahydro-1H-pyrrolizin-7a(5H)-yl)methoxy)-8-(6-methyl-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol (Ex-24). MS (ESI) [M+H]⁺ m/z 683. ¹H NMR (499 MHz, DMSO) δ 8.44 (d, J=5.4 Hz, 1H), 7.84 (d, J=5.4 Hz, 1H), 7.78 (s, 1H), 7.35 (s, 1H), 5.56 (s, 1H), 4.16-3.98 (m, 7H), 3.92-3.82 (m, 1H), 3.72-3.63 (m, 1H), 3.59-3.49 (m, 2H), 3.45 (d, J=12.4 Hz, 1H), 3.12-2.96 (m, 3H), 2.85-2.78 (m, 1H), 2.70 (s, 3H), 2.17-1.92 (m, 3H), 1.89-1.67 (m, 3H), 1.11 (s, 3H), 0.66 (t, J=6.9 Hz, 3H).

### Example 25: (6S)-4-(8-(5-ethynyl-6-fluoro-1H-benzo[f]indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-25)

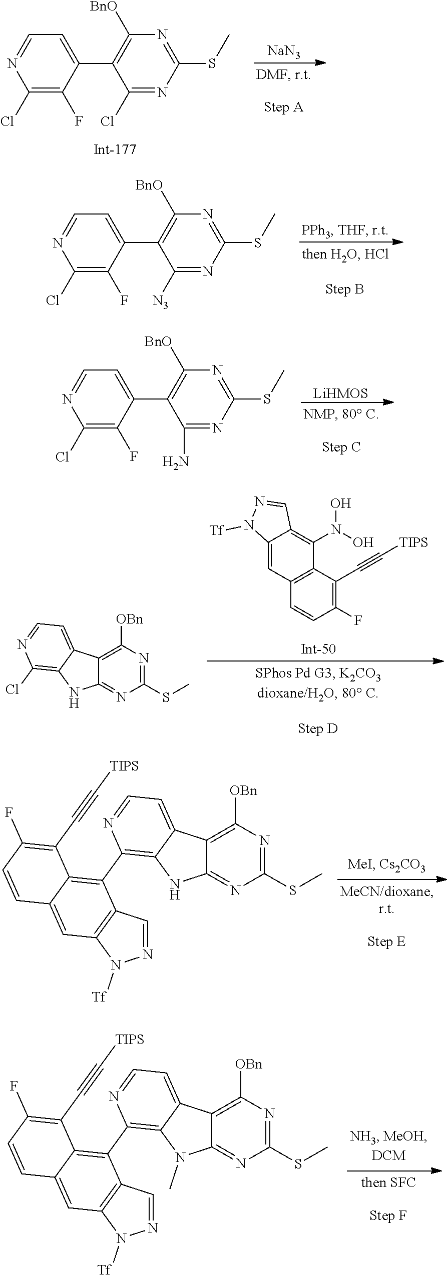

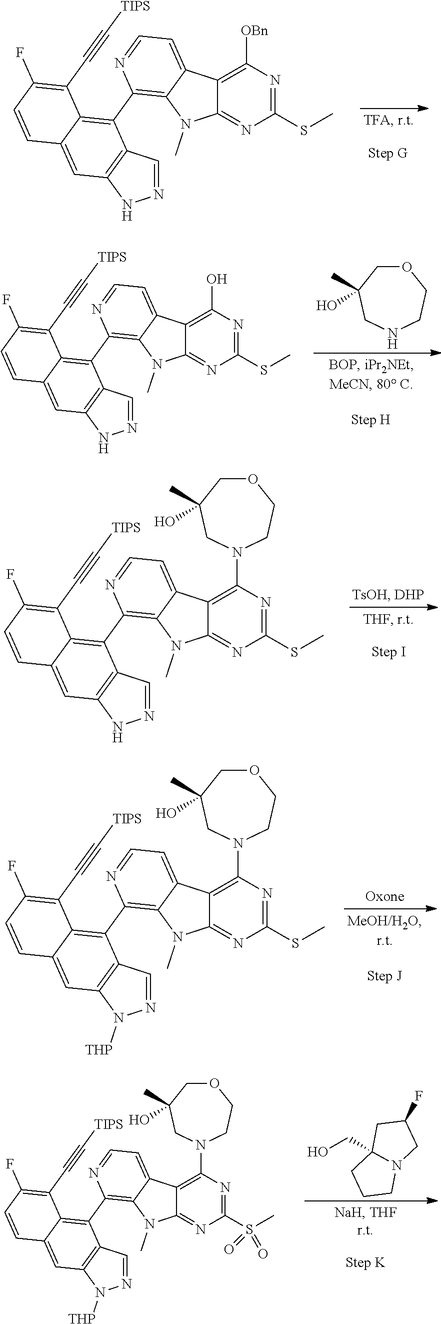

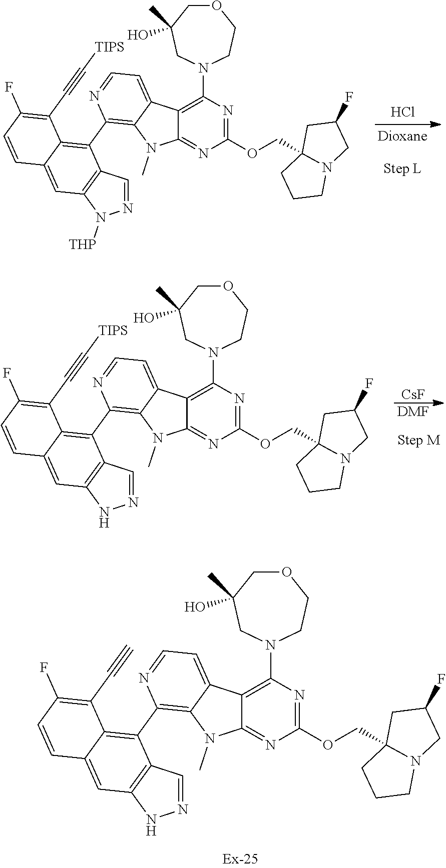

### Step A: 4-azido-6-(benzyloxy)-5-(2-chloro-3-fluoropyridin-4-yl)-2-(methylthio)pyrimidine

To a solution of 4-(benzyloxy)-6-chloro-5-(2-chloro-3-fluoropyridin-4-yl)-2-(methylthio)pyrimidine (Int-177) (10 g, 25 mmol) in DMF (168 mL) was added NaN₃ (1.68 g, 25.8 mmol) at 25° C. under N₂ and the mixture was stirred for 16 h. The reaction was quenched with H₂O (150 mL) and extracted with EtOAc (3×150 mL). The combined organic layers were dried over Na₂SO₄, filtered, and concentrated in vacuo to give 4-azido-6-(benzyloxy)-5-(2-chloro-3-fluoropyridin-4-yl)-2-(methylthio)pyrimidine. MS (ESI) [M+H]⁺: m/z 403.

### Step B: 6-(benzyloxy)-5-(2-chloro-3-fluoropyridin-4-yl)-2-(methylthio)pyrimidin-4-amine

To a solution of 4-azido-6-(benzyloxy)-5-(2-chloro-3-fluoropyridin-4-yl)-2-(methylthio)pyrimidine (10.17 g, 25.20 mmol) in THF (168 mL) was added triphenylphosphine (7.95 g, 30.3 mmol) at 25° C. under N₂ and the mixture was stirred for 1 h. To the reaction mixture was added H₂O (45.50 mL, 2525 mmol) and HCl (25.2 mL, 101 mmol, 4 M in dioxane) and the reaction mixture was stirred at room temperature for 16 h. The reaction mixture was quenched with sat. aq. NaHCO₃ (30 mL) and extracted with EtOAc (3×20 mL). The combined organic layers were dried over Na₂SO₄, filtered, and concentrated in vacuo. The crude material was purified by flash silica gel chromatography (0 to 46% EtOAc in petroleum ether) to give 6-(benzyloxy)-5-(2-chloro-3-fluoropyridin-4-yl)-2-(methylthio)pyrimidin-4-amine. MS (ESI) [M+H]⁺: m/z 377.

### Step C: 4-(benzyloxy)-8-chloro-2-(methylthio)-9H-pyrido[4′,3′:4,5]pyrrolo[2,3-d]pyrimidine

To a solution of 6-(benzyloxy)-5-(2-chloro-3-fluoropyridin-4-yl)-2-(methylthio)pyrimidin-4-amine (8.8 g, 23 mmol) in NMP (120 mL) was added LiHMDS (42.0 mL, 42.0 mmol, 1 M in THF) at 25° C. under N₂. The mixture was warmed to 80° C. and stirred for 16 h. The reaction was cooled to room temperature and poured into H₂O (200 mL). The reaction was filtered and the filter cake was washed with MeOH (20 mL). The filtrate was concentrated in vacuo to give crude 4-(benzyloxy)-8-chloro-2-(methylthio)-9H-pyrido[4′,3′:4,5]pyrrolo[2,3-d]pyrimidine. MS (ESI) [M+H]⁺: m/z 357.

### Step D: 4-(benzyloxy)-8-(6-fluoro-1-((trifluoromethyl) sulfonyl)-5-((triisopropylsilyl)ethynyl)-1H-benzo[f]indazol-4-yl)-2-(methylthio)-9H-pyrido[4′,3′:4,5]pyrrolo[2,3-d]pyrimidine

To a solution of 4-(benzyloxy)-8-chloro-2-(methylthio)-9H-pyrido[4′,3′:4,5]pyrrolo[2,3-d]pyrimidine (2.0 g, 5.6 mmol) in dioxane (20 mL) was added (6-fluoro-1-((trifluoromethyl) sulfonyl)-5-((triisopropylsilyl)ethynyl)-1H-benzo[f]indazol-4-yl)boronic acid (Int-59) (0.912 g, 1.681 mmol), K₂CO₃ (8.41 mL, 16.81 mmol, 2 M in H₂O) and (2-dicyclohexylphosphino-2′,6′-dimethoxybiphenyl) [2-(2′-amino-1,1′-biphenyl)]palladium (II) methanesulfonate (2.187 g, 2.800 mmol) at 25° C. The reaction mixture was warmed to 80° C. and stirred for 1 h under N₂. Then to the reaction mixture was added an additional solution of (6-fluoro-1-((trifluoromethyl) sulfonyl)-5-((triisopropylsilyl)ethynyl)-1H-benzo[f]indazol-4-yl)boronic acid (Int-59) (0.912 g, 1.68 mmol) in 1,4-dioxane (3 mL) each hour for 3 h. The reaction mixture was cooled to room temperature and diluted with H₂O (30 mL). The reaction mixture was extracted with EtOAc (3×50 mL) and the combined organic layers were dried over Na₂SO₄, filtered, and concentrated in vacuo. The crude mixture was purified by flash silica gel chromatography (0 to 26% EtOAc in petroleum ether) to give 4-(benzyloxy)-8-(6-fluoro-1-((trifluoromethyl) sulfonyl)-5-((triisopropylsilyl)ethynyl)-1H-benzo[f]indazol-4-yl)-2-(methylthio)-9H-pyrido[4′,3′:4,5]pyrrolo[2,3-d]pyrimidine. MS (ESI) [M+H]⁺: m/z 819.

### Step E: 4-(benzyloxy)-8-(6-fluoro-1-((trifluoromethyl)sulfonyl)-5-((triisopropylsilyl)ethynyl)-1H-benzo[f]indazol-4-yl)-9-methyl-2-(methylthio)-9H-pyrido[4′,3′:4,5]pyrrolo[2,3-d]pyrimidine

To a solution of Cs₂CO₃ (0.955 g, 2.93 mmol) in MeCN (8 mL) was added a solution of 4-(benzyloxy)-8-(6-fluoro-1-((trifluoromethyl) sulfonyl)-5-((triisopropylsilyl)ethynyl)-1H-benzo[f]indazol-4-yl)-2-(methylthio)-9H-pyrido[4′,3′:4,5]pyrrolo[2,3-d]pyrimidine (1.20 g, 1.46 mmol) in 1,4-dioxane (2 mL). The reaction mixture was stirred at 25° C. for 0.5 h and then a solution of MeI (0.101 mL, 1.61 mmol) in 1,4-dioxane (1 mL) was added at 25° C. under N₂. The mixture was stirred at 25° C. for 5 min. The reaction mixture was concentrated in vacuo and the residue was purified by flash silica gel chromatography (0 to 26% EtOAc in petroleum ether) to give 4-(benzyloxy)-8-(6-fluoro-1-((trifluoromethyl) sulfonyl)-5-((triisopropylsilyl)ethynyl)-1H-benzo[f]indazol-4-yl)-9-methyl-2-(methylthio)-9H-pyrido[4′,3′:4,5]pyrrolo[2,3-d]pyrimidine. MS (ESI) [M+H]⁺: m/z 833.

### Step F: 4-(benzyloxy)-8-(6-fluoro-5-((triisopropylsilyl)ethynyl)-1H-benzo[f]indazol-4-yl)-9-methyl-2-(methylthio)-9H-pyrido[4′,3′:4,5]pyrrolo[2,3-d]pyrimidine

To a solution of 4-(benzyloxy)-8-(6-fluoro-1-((trifluoromethyl) sulfonyl)-5-((triisopropylsilyl)ethynyl)-1H-benzo[f]indazol-4-yl)-9-methyl-2-(methylthio)-9H-pyrido[4′,3′:4,5]pyrrolo[2,3-d]pyrimidine (850 mg, 1.02 mmol) in DCM (2 mL) was added ammonia (8.0 mL, 56 mmol, 7 M in MeOH) at 25° C. under N₂ atmosphere. The mixture was stirred at 25° C. for 16 h. The reaction was concentrated in vacuo to give racemic 4-(benzyloxy)-8-(6-fluoro-5-((triisopropylsilyl)ethynyl)-1H-benzo[f]indazol-4-yl)-9-methyl-2-(methylthio)-9H-pyrido[4′,3′:4,5]pyrrolo[2,3-d]pyrimidine. The racemic 4-(benzyloxy)-8-(6-fluoro-5-((triisopropylsilyl)ethynyl)-1H-benzo[f]indazol-4-yl)-9-methyl-2-(methylthio)-9H-pyrido[4′,3′:4,5]pyrrolo[2,3-d]pyrimidine was separated by preparative chiral SFC (Column O, 55% EtOH w/0.1% NH₄OH) to give 4-(benzyloxy)-8-(6-fluoro-5-((triisopropylsilyl)ethynyl)-1H-benzo[f]indazol-4-yl)-9-methyl-2-(methylthio)-9H-pyrido[4′,3′:4,5]pyrrolo[2,3-d]pyrimidine (the second eluting isomer from SFC). MS (ESI) [M+H]⁺: m/z 701.

### Step G: 8-(6-fluoro-5-((triisopropylsilyl)ethynyl)-1H-benzo[f]indazol-4-yl)-9-methyl-2-(methylthio)-9H-pyrido[4′,3′:4,5]pyrrolo[2,3-d]pyrimidin-4-ol

A solution of 4-(benzyloxy)-8-(6-fluoro-5-((triisopropylsilyl)ethynyl)-1H-benzo[f]indazol-4-yl)-9-methyl-2-(methylthio)-9H-pyrido[4′,3′:4,5]pyrrolo[2,3-d]pyrimidine (270 mg, 0.385 mmol) in TFA (5 mL) was stirred at 25° C. for 1 h. TFA was removed in vacuo and the residue was basified with sat. aq. NaHCO₃ (5 mL), filtered, and the filter cake was collected and dried in vacuum to afford 8-(6-fluoro-5-((triisopropylsilyl)ethynyl)-1H-benzo[f]indazol-4-yl)-9-methyl-2-(methylthio)-9H-pyrido[4′,3′:4,5]pyrrolo[2,3-d]pyrimidin-4-ol. MS (ESI) [M+H]⁺: m/z 611.

### Step H: (6S)-4-(8-(16-fluoro-5-(triisopropylsilyl)ethynyl)-1H-benzo[f]indazol-4-yl)-9-methyl-2-(methylthio)-9H-pyrido[4′,3′:4,5]pyrrolo[2,3-d]pyrimidin-4-yl)-6-methyl-1,4-oxazepan-6-ol

To a solution of 8-(6-fluoro-5-((triisopropylsilyl)ethynyl)-1H-benzo[f]indazol-4-yl)-9-methyl-2-(methylthio)-9H-pyrido[4′,3′:4,5]pyrrolo[2,3-d]pyrimidin-4-ol (80 mg, 0.13 mmol) in MeCN (2 mL) was added BOP (87.0 mg, 0.196 mmol) at 25° C. and the reaction mixture was stirred for 0.5 h under N₂. Then to the reaction mixture was added (S)-6-methyl-1,4-oxazepan-6-ol hydrochloride (32.9 mg, 0.196 mmol) and N,N-diisopropylethylamine (0.137 mL, 0.786 mmol) at 25° C. The reaction was warmed to 80° C. and stirred for 16 h. The reaction was cooled to room temperature and concentrated in vacuo. The crude residue was purified by preparative TLC (SiO₂, petroleum ether/EtOAc=1/1) to give (6S)-4-(8-(6-fluoro-5-((triisopropylsilyl)ethynyl)-1H-benzo[f]indazol-4-yl)-9-methyl-2-(methylthio)-9H-pyrido[4′,3′:4,5]pyrrolo[2,3-d]pyrimidin-4-yl)-6-methyl-1,4-oxazepan-6-ol. MS (ESI) [M+H]⁺: m/z 724.

### Step I: (6S)-4-(8-(6-fluoro-1-(tetrahydro-2H-pyran-2-yl)-5-((triisopropylsilyl)ethynyl)-1H-benzo[f]indazol-4-yl)-9-methyl-2-(methylthio)-9H-pyrido[4′,3′:4,5]pyrrolo[2,3-d]pyrimidin-4-yl)-6-methyl-1,4-oxazepan-6-ol

To a solution of (6S)-4-(8-(6-fluoro-5-((triisopropylsilyl)ethynyl)-1H-benzo[f]indazol-4-yl)-9-methyl-2-(methylthio)-9H-pyrido[4′,3′:4,5]pyrrolo[2,3-d]pyrimidin-4-yl)-6-methyl-1,4-oxazepan-6-ol (70 mg, 0.097 mmol) in THF (0.3 mL) was added DHP (0.088 mL, 0.97 mmol) and 4-methylbenzenesulfonic acid (16.65 mg, 0.09700 mmol) at 25° C. under N₂ and the mixture was stirred at room temperature for 1 h. The reaction mixture was purified directly by preparative TLC (petroleum ether/EtOAc=1:1) to give (6S)-4-(8-(6-fluoro-1-(tetrahydro-2H-pyran-2-yl)-5-((triisopropylsilyl)ethynyl)-1H-benzo[f]indazol-4-yl)-9-methyl-2-(methylthio)-9H-pyrido[4′,3′:4,5]pyrrolo[2,3-d]pyrimidin-4-yl)-6-methyl-1,4-oxazepan-6-ol. MS (ESI) [M+H]⁺: m/z 808.

### Step J: (6S)-4-(8-(6-fluoro-1-(tetrahydro-2H-pyran-2-yl)-5-((triisopropylsilyl)ethynyl)-1H-benzo[f]indazol-4-yl)-9-methyl-2-(methylsulfonyl)-9H-pyrido[4′,3′:4,5]pyrrolo[2,3-d]pyrimidin-4-yl)-6-methyl-1,4-oxazepan-6-ol

To a solution of (6S)-4-(8-(6-fluoro-1-(tetrahydro-2H-pyran-2-yl)-5-((triisopropylsilyl)ethynyl)-1H-benzo[f]indazol-4-yl)-9-methyl-2-(methylthio)-9H-pyrido[4′,3′:4,5]pyrrolo[2,3-d]pyrimidin-4-yl)-6-methyl-1,4-oxazepan-6-ol (50 mg, 0.062 mmol) in MeOH (0.5 mL) and water (0.1 mL) was added oxone (114 mg, 0.186 mmol) at 25° C. under N₂ and the mixture was stirred for 1 h. The reaction mixture was quenched with sat. aq. Na₂SO₃ (2 mL), and extracted with DCM (3×3 mL). The combined organic layers were dried over Na₂SO₄, filtered, and concentrated in vacuo. The crude material was purified by preparative TLC (petroleum ether/EtOAc=1:1) to give (6S)-4-(8-(6-fluoro-1-(tetrahydro-2H-pyran-2-yl)-5-((triisopropylsilyl)ethynyl)-1H-benzo[f]indazol-4-yl)-9-methyl-2-(methylsulfonyl)-9H-pyrido[4′,3′:4,5]pyrrolo[2,3-d]pyrimidin-4-yl)-6-methyl-1,4-oxazepan-6-ol. MS (ESI) [M+H]⁺: m/z 840.

### Step K: (6S)-4-(8-(6-fluoro-1-(tetrahydro-2H-pyran-2-yl)-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol

To a solution of (6S)-4-(8-(6-fluoro-1-(tetrahydro-2H-pyran-2-yl)-5-((triisopropylsilyl)ethynyl)-1H-benzo[f]indazol-4-yl)-9-methyl-2-(methylsulfonyl)-9H-pyrido[4′,3′:4,5]pyrrolo[2,3-d]pyrimidin-4-yl)-6-methyl-1,4-oxazepan-6-ol (20 mg, 0.024 mmol) and ((2R,7aS)-2-fluorotetrahydro-1H-pyrrolizin-7a(5H)-yl) methanol (4.55 mg, 0.0290 mmol) in THF (0.5 mL) was added sodium hydride (4.76 mg, 0.119 mmol, 60% in mineral oil) under N₂. The reaction was stirred at room temperature for 0.5 h. The reaction was quenched with water (1 mL), extracted with EtOAc (2×10 mL), washed with brine (5 mL), dried over Na₂SO₄, filtered, and concentrated in vacuo. The crude residue was purified by preparative TLC (CH₂Cl₂:MeOH=20:1) to give (6S)-4-(8-(6-fluoro-1-(tetrahydro-2H-pyran-2-yl)-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol. MS (ESI) [M+H]⁺: m/z 919.

### Step L: (6S)-4-(8-(6-fluoro-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepam-6-ol

To a solution of (6S)-4-(8-(6-fluoro-1-(tetrahydro-2H-pyran-2-yl)-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (18 mg, 0.020 mmol) in MeOH (0.2 mL) was added HCl (0.5 mL, 4 M in dioxane). The mixture was stirred at room temperature for 1 h. The solvent was evaporated under reduced pressure to give (6S)-4-(8-(6-fluoro-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol. MS (ESI) [M+H]⁺: m/z 835.

### Step M: (6S)-4-(8-(5-ethynyl-6-fluoro-1H-benzo[f]indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-25)

To a solution of (6S)-4-(8-(6-fluoro-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (16 mg, 0.019 mmol) in DMF (0.5 mL) was added cesium fluoride (29.1 mg, 0.192 mmol) under N₂. The mixture was stirred at room temperature for 15 h. The mixture was filtered and the filtrate was purified by preparative reverse-phase HPLC (MeCN/H₂O w/formic acid modifier) to give (6S)-4-(8-(5-ethynyl-6-fluoro-1H-benzo[f]indazol-4-yl)-2-(((2R,7aS)-2-fluorotetrahydro-1H-pyrrolizin-7a(5H)-yl)methoxy)-9-methyl-9H-pyrido[4′,3′:4,5]pyrrolo[2,3-d]pyrimidin-4-yl)-6-methyl-1,4-oxazepan-6-ol (Ex-25). MS (ESI) [M+H]⁺: m/z 679. ¹H NMR (400 MHz, MeOD) δ 8.49 (d, J=5.48 Hz, 1H), 8.39 (s, 1H), 8.26 (dd, J=5.96, 9.42 Hz, 1H), 8.00 (d, J=5.60 Hz, 1H), 7.70 (d, J=0.95 Hz, 1H), 7.43 (t, J=8.94 Hz, 1H), 5.46-5.27 (m, 1H), 4.61 (br s, 1H), 4.45 (br d, J=13.83 Hz, 1H), 4.36 (d, J=10.73 Hz, 1H), 4.29-4.21 (m, 2H), 4.09-3.98 (m, 2H), 3.92 (d, J=14.78 Hz, 1H), 3.88-3.82 (m, 1H), 3.74 (d, J=12.40 Hz, 1H), 3.51 (d, J=12.52 Hz, 1H), 3.38 (br s, 2H), 3.20 (d, J=0.83 Hz, 1H), 3.13-3.05 (m, 1H), 2.86 (s, 3H), 2.34-2.19 (m, 2H), 2.17-2.12 (m, 1H), 2.08-2.01 (m, 2H), 1.97-1.89 (m, 1H), 1.31-1.28 (m, 3H).

### Example 26: (6S)-4-(2-(((2R,7aS)-2-fluorotetrahydro-1H-pyrrolizin-7a(5H)-yl)methoxy)-6-methoxy-9-methyl-8-(6-methyl-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol (Ex-26)

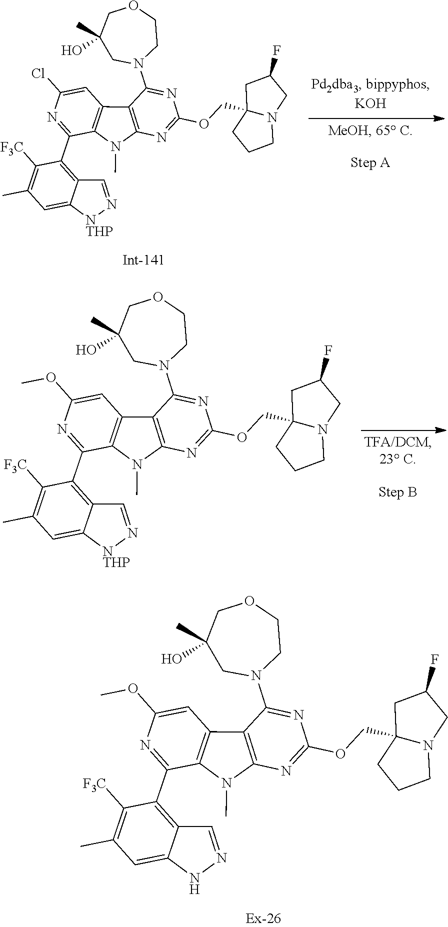

### Step A: (6S)-4-(2-(((2R,7aS)-2-fluorotetrahydro-1H-pyrrolizin-7a(5H)-yl)methoxy)-6-methoxy-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol

An oven-dried vial with a stir bar was charged with (6S)-4-(6-chloro-2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol (Int-141) (30 mg, 0.038 mmol), tris(dibenzylidineacetone) dipalladium (0) (6.98 mg, 7.62 μmol), 5-(di-tert-butylphosphaneyl)-1′,3′,5′-triphenyl-1′H-1,4′-bipyrazole (bippyphos) (7.72 mg, 0.0150 mmol), and potassium hydroxide (3.21 mg, 0.0570 mmol). The vial was fitted with a septum and placed under nitrogen. Methanol (3.81 mL) was added via a syringe and the reaction was heated to 65° C. for 2 h. LCMS indicated reaction completion. The reaction was cooled to r.t., filtered through CELITE®, and concentrated to afford crude (6S)-4-(2-(((2R,7aS)-2-fluorotetrahydro-1H-pyrrolizin-7a(5H)-yl)methoxy)-6-methoxy-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol, which was used directly in the next step without further purification. MS (ESI): m/z (M+H)⁺ 783.

### Step B: (6S)-4-(2-(((2R,7aS)-2-fluorotetrahydro-1H-pyrrolizin-7a(5H)-yl)methoxy)-6-methoxy-9-methyl-8-(6-methyl-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol (Ex-26)

A vial with a stir bar was charged with (6S)-4-(2-(((2R,7aS)-2-fluorotetrahydro-1H-pyrrolizin-7a(5H)-yl)methoxy)-6-methoxy-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol (29.8 mg, 0.0380 mmol). DCM (305 μL) and TFA (76 μL) were added via a syringe and the reaction was allowed to stir at r.t. for 1 h. After 1 h, the reaction was concentrated in vacuo. The residue was purified by preparative reverse-phase HPLC (MeCN/H₂O with TFA as modifier) to give (6S)-4-(2-(((2R,7aS)-2-fluorotetrahydro-1H-pyrrolizin-7a(5H)-yl)methoxy)-6-methoxy-9-methyl-8-(6-methyl-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol (Ex-26) as a 2TFA salt. MS (ESI): m/z (M+H)⁺ 699. ¹H NMR (500 MHz, de-MeOH) δ 7.75 (s, 1H), 7.46 (s, 1H), 7.24 (s, 1H), 5.61 (d, J=52.0 Hz, 1H), 4.70 (d, J=12.1 Hz, 1H), 4.57 (d, J=11.8 Hz, 1H), 4.41 (d, J=14.1 Hz, 1H), 4.17 (d, J=14.7 Hz, 1H), 4.06 (d, J=12.3 Hz, 1H), 4.00 (m, 1H), 3.95 (m, 4H), 3.91 (m, 3H), 3.88-3.80 (m, 1H), 3.75 (m, 2H), 3.48 (d, J=11.8 Hz, 2H), 3.12 (s, 3H), 2.77 (s, 3H), 2.65-2.55 (m, 1H), 2.46-2.30 (m, 3H), 2.19 (brs, 1H), 1.31 (s, 3H).

The example in the table below was synthesized using a similar procedure as described in the synthesis of Ex-26 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | | [M + H]⁺ |
|---|---|---|---|---|
| Ex. | Starting Material | Structure | Compound Name | found |
| Ex- 27 | 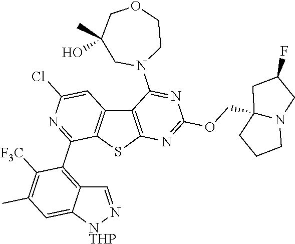 | 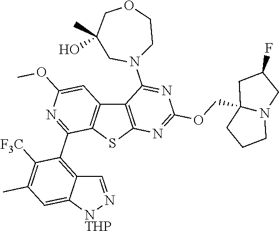 | (6S)-4-(2-(((2R,7aS)-2- fluorotetrahydro-1H- pyrrolizin-7a(5H)- yl)methoxy)-6-methoxy- 8-(6-methyl-5- (trifluoromethyl)-1H- indazol-4- yl)pyrido[4′,3′:4,5]thieno [2,3-d]pyrimidin-4-yl)-6- methyl-1,4-oxazepan-6- ol | 702 |
| | Int-142 | | | |

### Example 28: (6S)-4-(2-(((2R,7aS)-2-fluorotetrahydro-1H-pyrrolizin-7a(5H)-yl)methoxy)-6,9-dimethyl-8-(6-methyl-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol (Ex-28)

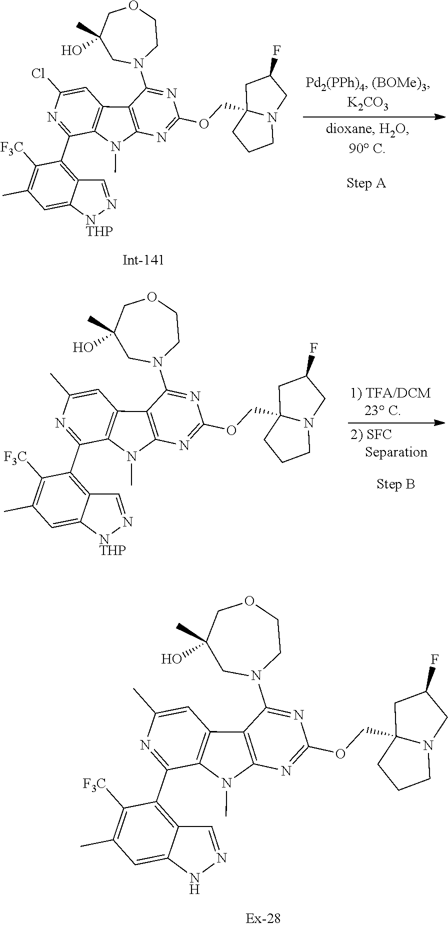

### Step A: (6S)-4-(2-(((2R,7aS)-2-fluorotetrahydro-1H-pyrrolizin-7a(5H)-yl)methoxy)-6,9-dimethyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol

A vial with a stir bar was charged with (6S)-4-(6-chloro-2-(((2R,7aS)-2-fluorotetrahydro-1H-pyrrolizin-7a(5H)-yl)methoxy)-9-methyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol (Int-141) (30 mg, 0.038 mmol), K₂CO₃ (26 mg, 0.191 mmol), and tetrakis(triphenylphosphine) palladium (0) (8.8 mg, 7.62 μmol). The vial was fitted with a septum and placed under nitrogen. Dioxane (508 μL), water (254 μL), and trimethylboroxine (26.6 μL, 0.19 mmol) were added via a syringe. The reaction was sparged with nitrogen for 15 min. The vial was placed in a preheated aluminum block set to 90° C. and the reaction was allowed to stir for 5 h. The reaction was cooled to r.t., diluted with water (5 mL) and EtOAc (5 mL). The layers were shaken and separated. The aqueous phase was extracted with EtOAc (2×5 mL) and the combined organics were washed with brine (15 mL). The combined organics were dried over sodium sulfate, filtered, and concentrated to obtain (6S)-4-(2-(((2R,7aS)-2-fluorotetrahydro-1H-pyrrolizin-7a(5H)-yl)methoxy)-6,9-dimethyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol, which was used in the subsequent step without further purification. MS (ESI): m/z (M+H)⁺ 767.

### Step B: (6S)-4-(2-(((2R,7aS)-2-fluorotetrahydro-1H-pyrrolizin-7a(5H)-yl)methoxy)-6,9-dimethyl-8-(6-methyl-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol (Ex-28)

A vial with a stir bar was charged with (6S)-4-(2-(((2R,7aS)-2-fluorotetrahydro-1H-pyrrolizin-7a(5H)-yl)methoxy)-6,9-dimethyl-8-(6-methyl-1-(tetrahydro-2H-pyran-2-yl)-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol (29 mg, 0.038 mmol). DCM (305 μL) and TFA (76 μL) were added and the reaction was allowed to stir at r.t. for 1 hour. After 1 hour, the reaction was concentrated in vacuo. The residue was purified by preparative reverse-phase HPLC (MeCN/H₂O with TFA as modifier) to give the product as a racemic mixture. The atropisomers were separated by SFC (Column C, 30% MeOH w/0.1% NH₄OH) to give (6S)-4-(2-(((2R,7aS)-2-fluorotetrahydro-1H-pyrrolizin-7a(5H)-yl)methoxy)-6,9-dimethyl-8-(6-methyl-5-(trifluoromethyl)-1H-indazol-4-yl)-9H-pyrido[4′,3′:4,5]pyrrolo[2,3-d]pyrimidin-4-yl)-6-methyl-1,4-oxazepan-6-ol (Ex-28) (the first eluting isomer from SFC). MS (ESI): m/z (M+H)⁺ 683. ¹H NMR (499 MHz, MeOD) δ 7.77 (s, 1H), 7.75 (s, 1H), 7.41 (s, 1H), 5.36 (d, J=53.4 Hz, 1H), 4.44 (d, J=13.8 Hz, 1H), 4.37 (d, J=10.7 Hz, 1H), 4.25 (d, J=10.8 Hz, 1H), 4.17 (d, J=14.8 Hz, 1H), 4.06 (m, 4H), 3.87 (d, J=14.9 Hz, 1H), 3.76-3.64 (m, 2H), 3.46 (d, J=12.5 Hz, 1H), 3.14 (s, 3H), 2.77 (s, 3H), 2.71 (s, 3H), 2.44-1.88 (m, 8H), 1.31 (s, 3H).

### Example 29: (6S)-4-(2-(((2R,7aS)-2-fluorotetrahydro-1H-pyrrolizin-7a(5H)-yl)methoxy)-6-(furan-3-yl)-8-(6-methyl-5-(trifluoromethyl)-1H-indazol-4-yl)pyrido[4′,3′:4,5]thieno[2,3-d]pyrimidin-4-yl)-6-methyl-1,4-oxazepan-6-ol (Ex-29)

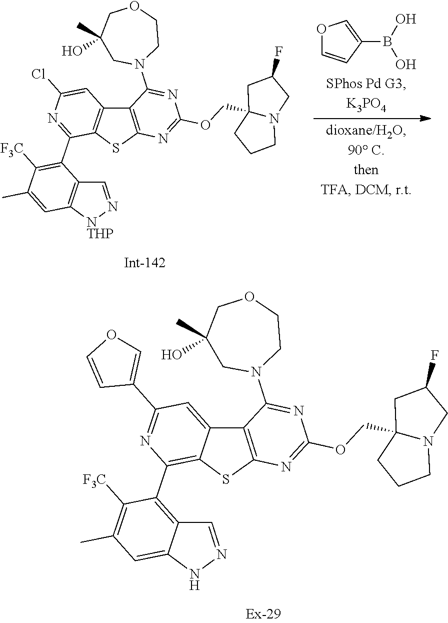

To a vial equipped with a stir bar was added SPhos Pd G3 (13.4 mg, 0.016 mmol), furan-3-ylboronic acid (10.6 mg, 0.0950 mmol), (6S)-4-(6-chloro-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidin-4-yl)-6-methyl-1,4-oxazepan-6-ol (Int-142) (25 mg, 0.032 mmol). The vial was fitted with a septum and dioxane (316 μL) and K₃PO₄ (95 μL, 0.095 mmol, 1 M in H₂O) was added. The reaction mixture was sparged with N₂ for 15 min. The reaction was then heated to 90° C. for 6 h. After 6 h, the reaction was cooled to room temperature and diluted with EtOAc (10 mL). The reaction was washed with water (10 mL), brine (10 mL), dried over Na₂SO₄, filtered, and concentrated. The reaction mixture was dissolved in DCM (1 mL) and TFA (0.3 mL) and allowed to stir at room temperature for 1 h. After 1 h, the reaction was quenched with sat. aq. NaHCO₃ (5 mL), and the layers were separated. The aqueous phase was washed with DCM (2×5 mL) and the combined organics were dried over Na₂SO₄, filtered, and concentrated. The crude product was purified by preparative reverse-phase HPLC (MeCN/H₂O w/TFA modifier) to afford (6S)-4-(2-(((2R,7aS)-2-fluorotetrahydro-1H-pyrrolizin-7a(5H)-yl)methoxy)-6-(furan-3-yl)-8-(6-methyl-5-(trifluoromethyl)-1H-indazol-4-yl)pyrido[4′,3′:4,5]thieno[2,3-d]pyrimidin-4-yl)-6-methyl-1,4-oxazepan-6-ol (Ex-29) as the TFA salt. MS (ESI): m/z (M+H)⁺ 738. ¹H NMR (500 MHz, d⁴-MeOD) δ 8.29 (s, 1H), 8.13 (s, 1H), 7.76 (s, 1H), 7.64 (s, 1H), 7.53 (s, 1H), 7.16 (s, 1H), 5.60 (d, J=51.2 Hz, 1H), 4.74-4.55 (m, 2H), 4.27-3.67 (m, 11H), 3.64-3.41 (m, 2H), 2.82-2.53 (m, 5H), 2.46-2.30 (m, 3H), 2.19 (m, 1H), 1.23 (d, J=15.3 Hz, 3H).

### Example 30: (6S)-4-(2-(((2R,7aS)-2-fluorotetrahydro-1H-pyrrolizin-7a(5H)-yl)methoxy)-8-(6-methyl-5-(trifluoromethyl)-1H-indazol-4-yl)-6-(methylamino)pyrido[4′,3′:4,5]thieno[2,3-d]pyrimidin-4-yl)-6-methyl-1,4-oxazepan-6-ol (Ex-30)

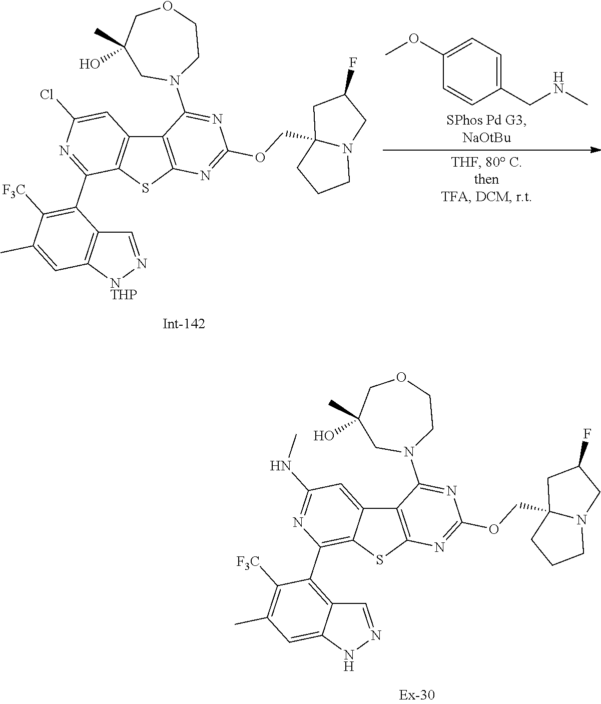

To a vial equipped with a stir bar were added SPhos Pd G3 (12.3 mg, 0.016 mmol), 4-methoxy-N-methylbenzylamine (5.22 μL, 0.035 mmol), (6S)-4-(6-chloro-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidin-4-yl)-6-methyl-1,4-oxazepan-6-ol (Int-142) (25 mg, 0.032 mmol). The vial was fitted with a septum and THF (1.05 mL) and sodium tert-butoxide (19.00 μL, 0.038 mmol, 2 M in THF) was added. The reaction mixture was sparged with N₂ for 15 min. The reaction was then heated to 80° C. for 2 h. After 2 h, the reaction was cooled to room temperature and diluted with EtOAc (10 mL). The reaction was washed with water (10 mL), brine (10 mL), dried over Na₂SO₄, filtered, and concentrated. The reaction mixture was dissolved in DCM (1 mL) and TFA (0.3 mL) and allowed to stir at room temperature for 1 h. After 1 h, the reaction was quenched with sat. aq. NaHCO₃ (5 mL) and the layers were separated. The aqueous phase was extracted with DCM (2×5 mL) and the combined organics were dried over Na₂SO₄, filtered, and concentrated. The crude product was purified by preparative reverse-phase HPLC (MeCN/H₂O w/TFA modifier) to afford (6S)-4-(2-(((2R,7aS)-2-fluorotetrahydro-1H-pyrrolizin-7a(5H)-yl)methoxy)-8-(6-methyl-5-(trifluoromethyl)-1H-indazol-4-yl)-6-(methylamino)pyrido[4′,3′:4,5]thieno[2,3-d]pyrimidin-4-yl)-6-methyl-1,4-oxazepan-6-ol (Ex-30) as the TFA salt. MS (ESI): m/z (M+H)⁺ 701. ¹H NMR (500 MHz, da-MeOD) δ 7.74 (s, 1H), 7.61 (s, 1H), 6.95 (s, 1H), 5.59 (d. J=50.9 Hz, 1H), 4.72-4.51 (m, 2H), 4.27-3.68 (m, 11H), 3.58-3.38 (m, 2H), 3.02 (s, 3H), 2.66 (m, 5H), 2.51-2.04 (m, 4H), 1.22 (d. J=11.3 Hz, 3H).

### Example 31: 2-(((2R,7aS)-2-fluorotetrahydro-1H-pyrrolizin-7a(5H)-yl)methoxy)-4-((S)-6-hydroxy-6-methyl-1,4-oxazepan-4-yl)-8-(6-methyl-5-(trifluoromethyl)-1H-indazol-4-yl)pyrido[4′,3′:4,5]thieno[2,3-d]pyrimidine-6-carbonitrile (Ex-31)

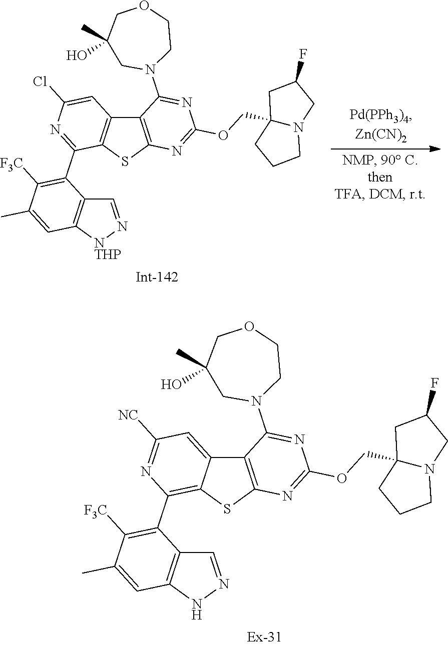

A vial with stir bar was charged with (6S)-4-(6-chloro-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidin-4-yl)-6-methyl-1,4-oxazepan-6-ol (Int-142) (25 mg, 0.032 mmol), tetrakis(triphenylphosphine) palladium (0) (18.3 mg, 0.016 mmol), and zinc cyanide (11.1 mg, 0.095 mmol). The vial was fitted with a septum and the reaction flask was flushed with N₂. NMP (1.05 mL) was added via syringe and the reaction was heated to 90° C. for 5 h. After 5 h, the reaction was cooled to room temperature and diluted with EtOAc (20 mL) and water (10 mL). The layers were shaken and separated. The organic phase was washed with water (10 mL), brine (10 mL), dried over Na₂SO₄, filtered, and concentrated. The crude reaction mixture was dissolved in DCM (1 mL) and TFA (0.3 mL) and allowed to stir at room temperature for 1 h. After 1 h, the reaction was quenched with sat. aq. NaHCO₃, and the layers were separated. The aqueous phase was washed with DCM (2×5 mL) and the combined organics were dried over Na₂SO₄, filtered, and concentrated. The crude product was purified by preparative reverse-phase HPLC (MeCN/H₂O w/TFA modifier) to afford 2-(((2R,7aS)-2-fluorotetrahydro-1H-pyrrolizin-7a(5H)-yl)methoxy)-4-((S)-6-hydroxy-6-methyl-1,4-oxazepan-4-yl)-8-(6-methyl-5-(trifluoromethyl)-1H-indazol-4-yl)pyrido[4′,3′:4,5]thieno[2,3-d]pyrimidine-6-carbonitrile (Ex-31) as the TFA salt. MS (ESI): m/z (M+H)⁺ 697. ¹H NMR (500 MHz, d⁴-MeOD) δ 8.41 (d, J=4.1 Hz, 1H), 7.80 (s, 1H), 7.55 (d, J=10.0 Hz, 1H), 5.59 (d, J=52.0 Hz, 1H), 4.77-4.54 (m, 2H), 4.29-3.67 (m, 10H), 3.52 (m, 2H), 2.85-2.55 (m, 5H), 2.51-2.28 (m, 3H), 2.18 (s, 1H), 1.20 (d, J=15.2 Hz, 3H).

### Example 32: (6S)-4-(8-(5-chloro-6-methyl-1H-indazol-4-yl)-2-(((2R,7aS)-2-fluorotetrahydro-1H-pyrrolizin-a (5H)-yl)methoxy)pyrido[4′,3′:4,5]thieno[2,3-d]pyrimidin-4-yl)-6-methyl-1,4-oxazepan-6-ol (Ex-32)

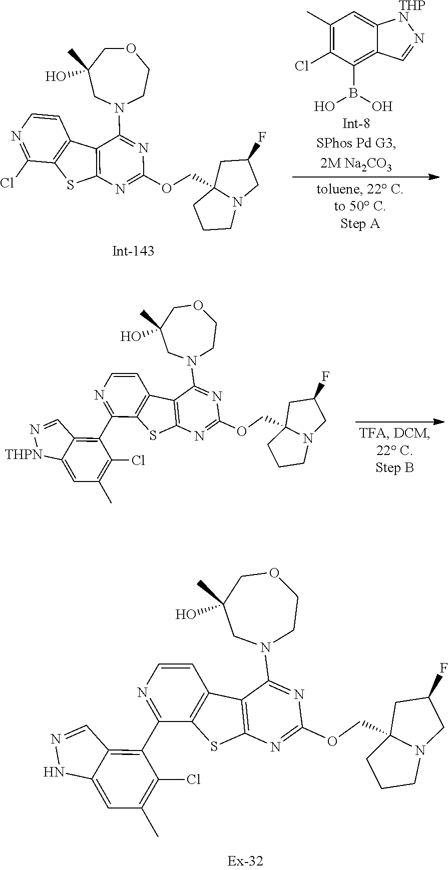

### Step A: (6S)-4-(8-(5-chloro-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol

To a mixture of(S)-4-(8-chloro-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Int-143) (30 mg, 0.059 mmol), (5-chloro-6-methyl-1-(tetrahydro-2H-pyran-2-yl)-3a,7a-dihydro-1H-indazol-4-yl)boronic acid (Int-8) (26 mg, 0.089 mmol), and SPhos Pd G3 (23 mg, 0.030 mmol) in Toluene (1 ml) was added 2M aq. sodium carbonate (0.089 ml, 0.18 mmol). The solution was degassed three times with nitrogen and vacuum and stirred at room temperature for 30 min. LCMS showed major product formation. The reaction was then heated to 50° C. for another 20 min. The reaction was cooled to room temperature and water and EtOAc were added. The layers were mixed and the organic layer was separated. The organic layer was washed with brine and concentrated. The product was purified by silica gel chromatography (0-50% MeOH in DCM) to obtain (6S)-4-(8-(5-chloro-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol. MS (ESI): m/z (M+H)⁺ 722.

### Step B: (6S)-4-(8-(5-chloro-6-methyl-1H-indazol-4-yl)-2-(((2R,7aS)-2-fluorotetrahydro-1H-pyrrolizin-a(5H)-yl)methoxy)pyrido[4′,3′:4,5]thieno[2,3-d]pyrimidin-4-yl)-6-methyl-1,4-oxazepan-6-ol (Ex-32)

To a solution of (6S)-4-(8-(5-chloro-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (25 mg, 0.035 mmol) in DCM (1 ml) was added TFA (1 ml). The reaction was stirred at room temperature for 30 mins. The reaction mixture was concentrated in vacuo and purified by reverse-phase preparative HPLC (MeCN/H₂O w/NH₄OH modifier) to obtain (6S)-4-(8-(5-chloro-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-32). MS (ESI): m/z (M+H)⁺ 638. ¹H NMR (600 MHz, DMSO-d₆) δ 13.36 (s, 1H), 8.81 (d, J=5.4 Hz, 1H), 7.87 (d, J=3.8 Hz, 1H), 7.74 (s, 1H), 7.51 (s, 1H), 5.31 (s, 1H), 5.21 (d, J=9.3 Hz, 1H), 5.14 (s, 1H), 4.07 (dd, J=44.4, 10.0 Hz, 3H), 4.01-3.84 (m, 6H), 3.54 (dd, J=12.1, 8.7 Hz, 1H), 3.44 (dd, J=12.3, 3.0 Hz, 1H), 3.06 (d, J=10.2 Hz, 2H), 3.02 (d, J=11.6 Hz, 2H), 2.87-2.78 (m, 1H), 2.57 (s, 3H), 2.14-2.02 (m, 2H), 1.97 (s, 1H), 1.89-1.70 (m, 4H), 1.02 (d, J=10.5 Hz, 3H).

### Example 33: (6S)-4-(8-(5-ethynyl-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-33)

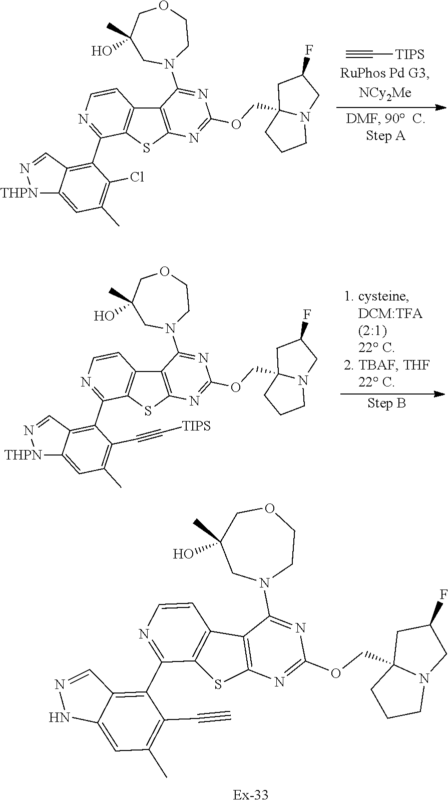

### Step A: (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-5-((triisopropylsilyl)ethynyl)-1H-indazol-4-yl)pyrido[4′,3′:4,5]thieno[2,3-d]pyrimidin-4-yl)-6-methyl-1,4-oxazepan-6-ol

A vial was loaded with ((6S)-4-(8-(5-chloro-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (102 mg, 0.100 mmol), RuPhos Pd G3 (42 mg, 0.050 mmol) and DMF (1 mL) under nitrogen. Ethynyltriisopropylsilane (222 μL, 0.990 mmol) and N-cyclohexyl-N-methylcyclohexanamine (127 μL, 0.593 mmol) were added via a syringe and the mixture was heated to 90° C. for 16 h. After 16 h, the reaction was quenched with water, extracted with DCM, and evaporated under reduced pressure. The mixture was purified by silica gel chromatography (DCM/MeOH, 8:1) to afford (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-5-((triisopropylsilyl)ethynyl)-1H-indazol-4-yl)pyrido[4′,3′:4,5]thieno[2,3-d]pyrimidin-4-yl)-6-methyl-1,4-oxazepan-6-ol. MS (ESI): m/z (M+H)⁺ 868.

### Step B: (6S)-4-(8-(5-ethynyl-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-33)

A vial was loaded with (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-5-((triisopropylsilyl)ethynyl)-1H-indazol-4-yl)pyrido[4′,3′:4,5]thieno[2,3-d]pyrimidin-4-yl)-6-methyl-1,4-oxazepan-6-ol (50 mg, 0.058 mmol) and L-cysteine (13.9 mg, 0.120 mmol). 2 mL of DCM (2 mL) and TFA (1 mL) were added via a syringe. The mixture was allowed to stir for 30 min. The volatiles were evaporated, and the mixture was redissolved in DCM and sat. aq. NaHCO₃. The mixture was extracted with DCM, dried over Na₂SO₄, filtered, and evaporated under reduced pressure. The crude material was dissolved in THF (1 mL), and tetrabutylammonium fluoride was added via a syringe (0.203 mL, 0.203 mmol, 1M in THF). The mixture was stirred at 22° C. for 2 hours. The mixture was diluted with EtOAc and water, and the aqueous layer was separated and extracted with EtOAc. The organic phase was washed with brine and dried with Na₂SO₄. The mixture was filtered and the filtrate was evaporated under reduced pressure. The crude product was purified via preparative reverse-phase HPLC (MeCN/H₂O with TFA modifier) to afford (6S)-4-(8-(5-ethynyl-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-33) as a TFA salt. MS (ESI): m/z (M+H)⁺ 628. ¹H NMR (600 MHz, MeOD) δ 8.85 (dd, J=6.0, 2.9 Hz, 1H), 8.12 (dd, J=6.0, 3.7 Hz, 1H), 7.78-7.68 (m, 2H), 5.72-5.51 (m, 1H), 4.72 (dd, J=11.9, 2.6 Hz, 1H), 4.65 (d, J=11.9 Hz, 1H), 4.28-4.20 (m, 1H), 4.14 (dd, J=14.6, 4.0 Hz, 1H), 4.08-3.99 (m, 2H), 4.00-3.90 (m, 5H), 3.91-3.83 (m, 1H), 3.74 (dd, J=12.6, 2.8 Hz, 1H), 3.65 (d, J=5.8 Hz, 1H), 3.58-3.51 (m, 1H), 3.52-3.44 (m, 1H), 2.77-2.57 (m, 5H), 2.48-2.40 (m, 1H), 2.40-2.32 (m, 2H), 2.27-2.16 (m, 1H), 1.26-1.14 (m, 3H).

### Example 34:4-(2-(((2R,7aS)-2-fluorotetrahydro-1H-pyrrolizin-7a(5H)-yl)methoxy)-4-((S)-6-hydroxy-6-methyl-1,4-oxazepan-4-yl)pyrido[4′,3′:4,5]thieno[2,3-d]pyrimidin-8-yl)-6-methyl-1H-indazole-5-carbonitrile (Ex-34)

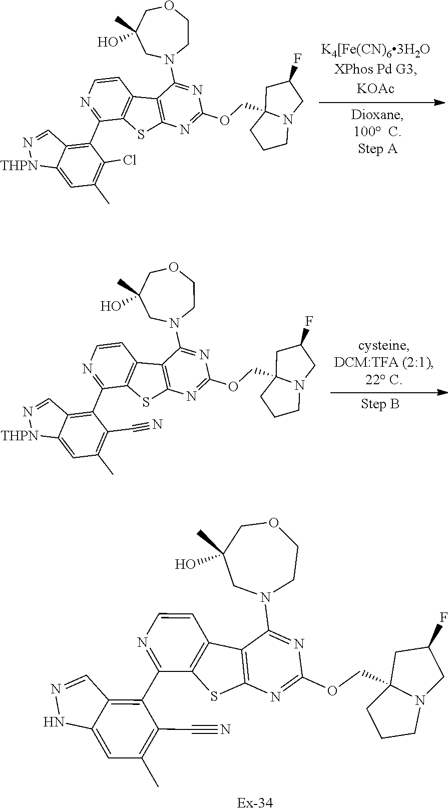

### Step A: 4-(2-(((2R,7aS)-2-fluorotetrahydro-1H-pyrrolizin-7a(5H)-yl)methoxy)-4-((S)-6-hydroxy-6-methyl-1,4-oxazepan-4-yl)pyrido[4′,3′:4,5]thieno[2,3-d]pyrimidin-8-yl)-6-methyl-1-(tetrahydro-2H-pyran-2-yl)-1H-indazole-5-carbonitrile

A vial was loaded with (6S)-4-(8-(5-chloro-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (120 mg, 0.0116 mmol), XPhos Pd G3 (9.9 mg, 0.012 mmol), potassium hexacyanoferrate (II) trihydrate (24.6 mg, 0.0580 mmol), and dioxane (2 mL) under nitrogen. Potassium acetate (0.050 M, 0.015 mmol) was added via a syringe as a degassed aqueous solution and the mixture was heated to 100° C. for 1 hour. LCMS showed formation of the desired product. The mixture was quenched with water and extracted with DCM, dried over Na₂SO₄, filtered, and evaporated under reduced pressure to afford 4-(2-(((2R,7aS)-2-fluorotetrahydro-1H-pyrrolizin-7a(5H)-yl)methoxy)-4-((S)-6-hydroxy-6-methyl-1,4-oxazepan-4-yl)pyrido[4′,3′:4,5]thieno[2,3-d]pyrimidin-8-yl)-6-methyl-1-(tetrahydro-2H-pyran-2-yl)-1H-indazole-5-carbonitrile. The crude mixture was used directly in the next step without further purification. MS (ESI): m/z (M+H)⁺ 713.

### Step B: 4-(2-(((2R,7aS)-2-fluorotetrahydro-1H-pyrrolizin-7a(5H)-yl)methoxy)-4-((S)-6-hydroxy-6-methyl-1,4-oxazepan-4-yl)pyrido[4′,3′:4,5]thieno[2,3-d]pyrimidin-8-yl)-6-methyl-1H-indazole-5-carbonitrile (Ex-34)

A vial was loaded with 4-(2-(((2R,7aS)-2-fluorotetrahydro-1H-pyrrolizin-7a(5H)-yl)methoxy)-4-((S)-6-hydroxy-6-methyl-1,4-oxazepan-4-yl)pyrido[4′,3′:4,5]thieno[2,3-d]pyrimidin-8-yl)-6-methyl-1-(tetrahydro-2H-pyran-2-yl)-1H-indazole-5-carbonitrile (83.0 mg, 0.116 mmol) and cysteine (14.0 mg, 0.116 mmol). The mixture was dissolved in DCM (2 mL) and TFA (1 mL) and stirred at r.t. for 30 min. LCMS showed starting material consumption and the desired product. The volatiles were evaporated and the crude product was neutralized with NaHCO₃, extracted with DCM. The organic phase was dried over Na₂SO₄, filtered, and evaporated under reduced pressure. The crude product mixture was subjected to preparative reverse phase HPLC purification (MeCN/H₂O with TFA modifier). A second purification was conducted by SFC purification (Column I, 25% MeOH with 0.1% NH₄OH) to afford 4-(2-(((2R,7aS)-2-fluorotetrahydro-1H-pyrrolizin-7a(5H)-yl)methoxy)-4-((S)-6-hydroxy-6-methyl-1,4-oxazepan-4-yl)pyrido[4′,3′:4,5]thieno[2,3-d]pyrimidin-8-yl)-6-methyl-1H-indazole-5-carbonitrile (Ex-34). MS (ESI): m/z (M+H)⁺ 629. ¹H NMR (600 MHz, MeOD) δ 8.83 (d, J=5.1 Hz, 1H), 7.98 (dd, J=5.5, 2.2 Hz, 1H), 7.85 (d, J=8.4 Hz, 1H), 7.77 (s, 1H), 5.42-5.19 (m, 1H), 4.32 (d, J=10.3 Hz, 1H), 4.23-4.14 (m, 2H), 4.10 (d, J=14.8 Hz, 1H), 4.05-3.97 (m, 1H), 3.97-3.84 (m, 3H), 3.83-3.77 (m, 1H), 3.72 (d, J=12.6 Hz, 1H), 3.49 (dd, J=12.5, 3.3 Hz, 1H), 3.37 (s, 1H), 3.29-3.23 (m, 2H), 3.21 (s, 1H), 3.08-3.01 (m, 1H), 2.77 (s, 3H), 2.38-2.15 (m, 2H), 2.14-2.07 (m, 1H), 2.05-1.96 (m, 2H), 1.95-1.86 (m, 1H), 1.22 (d, J=15.6 Hz, 3H).

### Example 35: (6S)-4-(2-(((2R,7aS)-2-fluorotetrahydro-1H-pyrrolizin-7a(5H)-yl)methoxy)-8-(6-methyl-5-vinyl-1H-indazol-4-yl)pyrido[4′,3′:4,5]thieno[2,3-d]pyrimidin-4-yl)-6-methyl-1,4-oxazepan-6-ol (Ex-35)

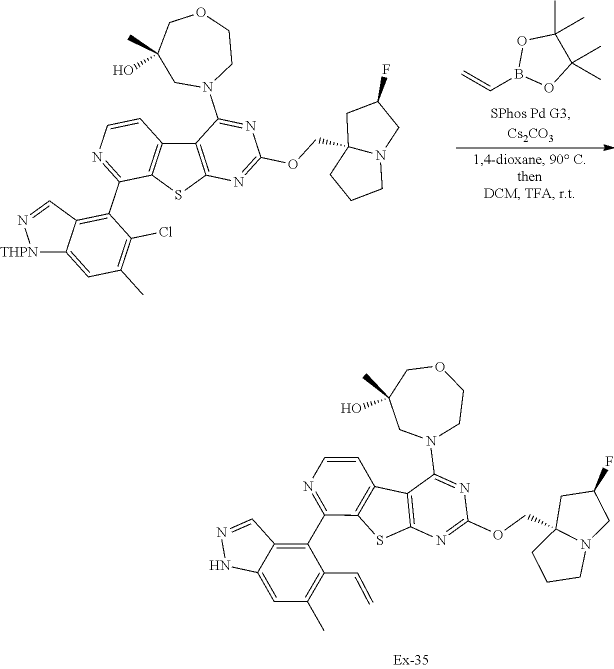

A mixture of (6S)-4-(8-(5-chloro-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (85.0 mg, 0.118 mmol), 4,4,5,5-tetramethyl-2-vinyl-1,3,2-dioxaborolane (0.080 mL, 0.47 mmol), cesium carbonate (115 mg, 0.353 mmol)) and SPhos Pd G3 (46 mg, 0.059 mmol) in 1,4-dioxane (3 mL) was evacuated and backfilled with nitrogen (3×). The reaction was heated to 90° C. and stirred for 2 h. The reaction mixture was cooled to r.t. and quenched with sat. aq. NH₄Cl solution and extracted with EtOAc. The organic layer was separated, washed with brine, and concentrated. The product was purified by silica gel chromatography (0 to 50% methanol in DCM) to give (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-5-vinyl-1H-indazol-4-yl)pyrido[4′,3′:4,5]thieno[2,3-d]pyrimidin-4-yl)-6-methyl-1,4-oxazepan-6-ol. This was taken in TFA (0.5 ml) and DCM (1 ml), stirred at r.t. for 45 mins. The reaction mixture was concentrated and purified by reverse-phase HPLC to obtain (6S)-4-(2-(((2R,7aS)-2-fluorotetrahydro-1H-pyrrolizin-7a(5H)-yl)methoxy)-8-(6-methyl-5-vinyl-1H-indazol-4-yl)pyrido[4′,3′:4,5]thieno[2,3-d]pyrimidin-4-yl)-6-methyl-1,4-oxazepan-6-ol (Ex-35). MS (ESI): m/z (M+H)⁺ 630. ¹H NMR (600 MHz, DMSO-d₆) δ 13.18 (s, 1H), 10.68 (s, 1H), 8.82 (d, J=5.5 Hz, 1H), 7.86 (t, J=5.1 Hz, 1H), 7.57 (s, 1H), 7.51 (d, J=6.7 Hz, 1H), 6.87-6.72 (m, 1H), 5.58 (d, J=51.9 Hz, 2H), 4.98 (d, J=11.2 Hz, 2H), 4.74 (d, J=17.6 Hz, 2H), 4.61-4.51 (m, 3H), 4.09-4.01 (m, 2H), 3.99-3.84 (m, 5H), 3.82-3.73 (m, 2H), 3.73-3.66 (m, 1H), 3.57-3.50 (m, 1H), 3.49-3.42 (m, 1H), 3.30 (s, 1H), 2.32 (s, 1H), 2.23-2.10 (m, 2H), 2.10-2.00 (m, 1H), 0.98 (d, J=15.3 Hz, 3H).

### Example 36: (6S)-4-(8-(5-ethyl-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-36)

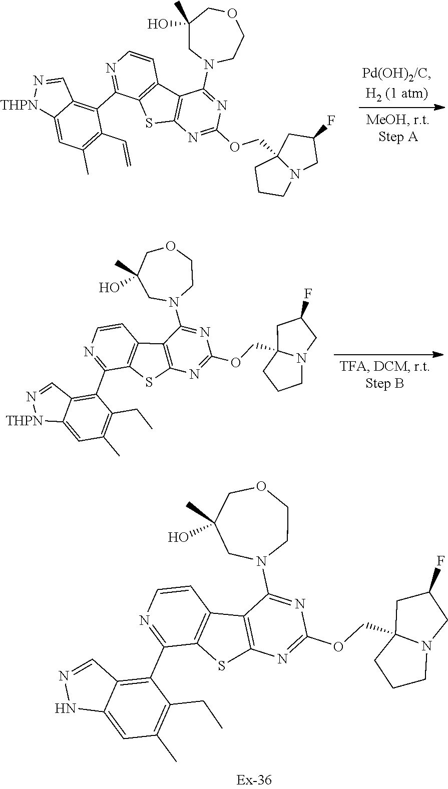

### Step A: (6S)-4-(8-(5-ethyl-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol

To a solution of (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-5-vinyl-1H-indazol-4-yl)pyrido[4′,3′:4,5]thieno[2,3-d]pyrimidin-4-yl)-6-methyl-1,4-oxazepan-6-ol (39 mg, 0.055 mmol) in MeOH (2 mL) was added palladium hydroxide on carbon (19.2 mg, 0.0270 mmol). The reaction was evacuated and backfilled with nitrogen 3 times and stirred under H₂ atmosphere at r.t. for 1 hr. After 1 hr, the reaction mixture was filtered and concentrated to give (6S)-4-(8-(5-ethyl-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol. MS (ESI): m/z (M+H)⁺ 716.

### Step B: (6S)-4-(8-(5-ethyl-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-36)

To a mixture of (6S)-4-(8-(5-ethyl-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (33 mg, 0.046 mmol) in DCM (1 ml) was added TFA (0.7 ml). The reaction was stirred at r.t. for 30 min, concentrated, and purified by reverse-phase preparative HPLC (MeCN/H₂O w/NH₄OH modifier) to provide (6S)-4-(8-(5-ethyl-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-36). MS (ESI): m/z (M+H)⁺ 632. ¹H NMR (600 MHz, DMSO-d₆) δ 13.03 (s, 1H), 8.79 (d, J=5.5 Hz, 1H), 7.83 (d, J=5.5 Hz, 1H), 7.52 (s, 1H), 7.32 (d, J=8.1 Hz, 1H), 5.31 (s, 1H), 5.23 (d, J=14.7 Hz, 2H), 4.17-3.98 (m, 3H), 3.91 (m, 4H), 3.49 (m, 2H), 3.04 (m, 3H), 2.93-2.61 (m, 2H), 2.36 (td, J=16.1, 13.7, 9.2 Hz, 1H), 2.22-1.89 (m, 4H), 1.89-1.63 (m, 4H), 1.01 (d, J=7.6 Hz, 3H), 0.94 (t, J=7.4 Hz, 3H).

### Example 37: (S)-4-(2-(((2R,7aS)-2-fluorotetrahydro-1H-pyrrolizin-7a(5H)-yl)methoxy)-8-(6-methyl-1H-indazol-4-yl)pyrido[4′,3′:4,5]thieno[2,3-d]pyrimidin-4-yl)-6-methyl-1,4-oxazepan-6-ol (Ex-37)

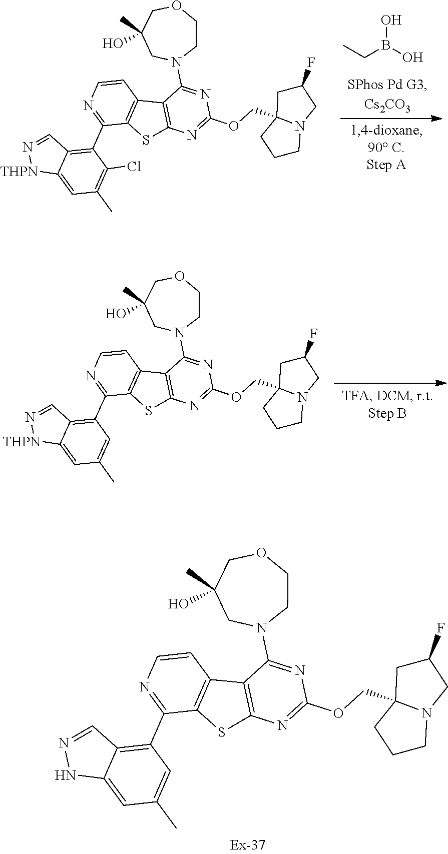

### Step A: (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-1H-indazol-4-yl)pyrido[4′,3′:4,5]thieno[2,3-d]pyrimidin-4-yl)-6-methyl-1,4-oxazepan-6-ol

A mixture of (6S)-4-(8-(5-chloro-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (from Step A of Example 25) (75 mg, 0.066 mmol), ethylboronic acid (19.6 mg, 0.266 mmol), cesium carbonate (65 mg, 0.199 mmol) and SPhos Pd G3 (25.9 mg, 0.033 mmol) in 1,4-dioxane (3 ml) was degassed by cycling nitrogen and vacuum atmosphere (3×) and stirred at 90° C. for 2 hr. After 2 hr, the reaction was cooled to r.t. and diluted with water and EtOAc. The organic layer was separated, washed with brine, and concentrated. The product was purified by silica gel chromatography (0 to 100% EtOAc in hexane) to provide (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-1H-indazol-4-yl)pyrido[4′,3′:4,5]thieno[2,3-d]pyrimidin-4-yl)-6-methyl-1,4-oxazepan-6-ol. MS (ESI): m/z (M+H)⁺ 688.

### Step B: (S)-4-(2-(((2R,7aS)-2-fluorotetrahydro-1H-pyrrolizin-7a(5H)-yl)methoxy)-8-(6-methyl-1H-indazol-4-yl)pyrido[4′,3′:4,5]thieno[2,3-d]pyrimidin-4-yl)-6-methyl-1,4-oxazepan-6-ol (Ex-37)

To a mixture of (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-1H-indazol-4-yl)pyrido[4′,3′:4,5]thieno[2,3-d]pyrimidin-4-yl)-6-methyl-1,4-oxazepan-6-ol (28.8 mg, 0.042 mmol) in DCM (1 mL) was added TFA (0.5 ml). The reaction was stirred at r.t. for 30 mins, concentrated, and purified by reverse phase preparative HPLC (MeCN/H₂O w/NH₄OH modifier) to provide(S)-4-(2-(((2R,7aS)-2-fluorotetrahydro-1H-pyrrolizin-7a(5H)-yl)methoxy)-8-(6-methyl-1H-indazol-4-yl)pyrido[4′,3′:4,5]thieno[2,3-d]pyrimidin-4-yl)-6-methyl-1,4-oxazepan-6-ol (Ex-37). MS (ESI): m/z (M+H)⁺ 604. ¹H NMR (600 MHz, DMSO-d₆) δ 13.14 (s, 1H), 8.83 (s, 1H), 8.13 (s, 1H), 7.79 (d, J=5.4 Hz, 1H), 7.55-7.47 (m, 2H), 5.28 (d. J=52.8 Hz, 1H), 5.12 (s, 1H), 4.19-4.12 (m, 1H), 4.10-4.04 (m, 1H), 3.96-3.89 (m, 3H), 3.89-3.81 (m, 2H), 3.53 (d, J=12.2 Hz, 1H), 3.41 (d, J=12.2 Hz, 1H), 3.11-3.05 (m, 2H), 3.05-2.99 (m, 1H), 2.88-2.79 (m, 1H), 2.57 (s, 3H), 2.19-2.04 (m, 2H), 1.99 (s, 1H), 1.89-1.83 (m, 1H), 1.82-1.74 (m, 2H), 1.24 (s, 1H), 1.00 (s, 3H).

### Example 38: (6S)-4-(8-(5-ethoxy-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-38)

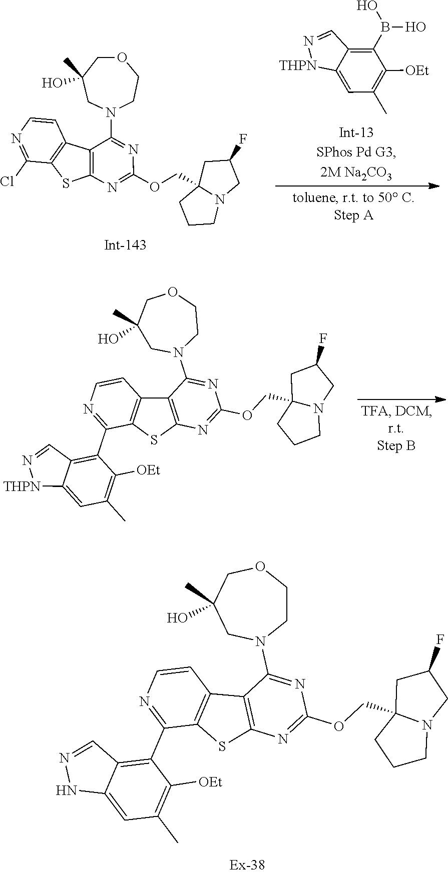

### Step A: (6S)-4-(8-(5-ethoxy-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol

To a mixture of(S)-4-(8-chloro-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Int-143) (100 mg, 0.197 mmol)), 5-ethoxy-6-methyl-1-(tetrahydro-2H-pyran-2-yl)-1H-indazol-4-yl)boronic acid (Int-13) (90 mg, 0.295 mmol), and SPhos Pd G3 (77 mg, 0.098 mmol) in toluene (3 ml) was added sodium carbonate (0.295 ml, 0.591 mmol, 2 M in H₂O).

The reaction was degassed by cycling nitrogen and vacuum atmosphere (3×) and stirred at r.t. for 30 min. The reaction was heated to 50° C. for another 20 min. The reaction was cooled to r.t. and diluted with water and EtOAc. The organic layer was separated, washed with brine, and concentrated. The product was purified by silica gel chromatography (0 to 50% MeOH in DCM) to provide (6S)-4-(8-(5-ethoxy-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol. MS (ESI): m/z (M+H)⁺ 732.

### Step B: (6S)-4-(8-(5-ethoxy-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-38)

To a solution of (6S)-4-(8-(5-ethoxy-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (65 mg, 0.089 mmol) in DCM (1 ml) was added TFA (0.029 ml, 0.444 mmol). The reaction was stirred at r.t. for 30 min, concentrated in vacuo, and purified by reverse phase preparative HPLC (MeCN/H₂O w/NH₄OH modifier) to provide (6S)-4-(8-(5-ethoxy-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-38). MS (ESI): m/z (M+H)⁺ 648. ¹H NMR (600 MHz, DMSO-d₆) δ 13.07 (s, 1H), 8.81 (d, J=5.4 Hz, 1H), 7.83 (d, J=5.5 Hz, 1H), 7.60 (s, 1H), 7.55 (s, 1H), 5.27 (d, J=54.1 Hz, 1H), 5.17 (s, 1H), 4.12 (d, J=10.1 Hz, 1H), 4.05 (d, J=10.3 Hz, 2H), 3.90 (s, 5H), 3.53-3.41 (m, 3H), 3.06 (m, 3H), 2.88-2.78 (m, 1H), 2.46 (s, 3H), 2.21-1.93 (m, 3H), 1.93-1.67 (m, 3H), 0.96 (s, 3H), 0.86 (t, J=7.0 Hz, 3H).

The examples in the table below were synthesized using a similar procedure as described in the synthesis of Ex-38 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | | SFC | |
|---|---|---|---|---|---|
| | | | | Separa- | |
| | Starting | | | tion | [M + |
| Ex. | Material | Structure | Compound Name | Conditions | H]⁺ |
| Ex- 39 | 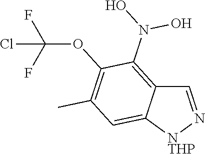 Int-12 | 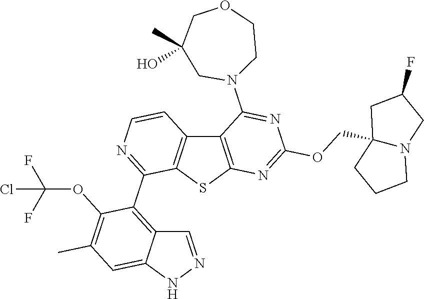 [SMILES: Cc1cc2[nH]ncc2c(-c2nccc3c2sc2nc(OC[C@@]45CCCN4C[C@H](F)C5)nc(N4CCOC[C@@](C)(O)C4)c23)c1OC(F)(F)Cl] | (6S)-4-(8-(5- (chlorodifluoromethoxy)- 6-methy1-1H-indazol- 4-yl)-2-(((2R,7aS)-2- fluorotetrahydro-1H- pyrrolizin-7a(5H)- yl)methoxy)pyri- do[4′,3′:4,5]thi- eno[2,3- d]pyrimidin-4-yl)-6- methyl-1,4-oxazepan-6- ol | | 704 |
| Ex- 40 | 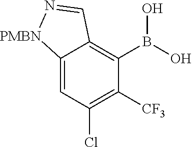 Int-57 | 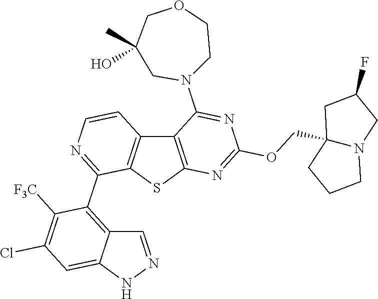 [SMILES: C[C@@]1(O)COCCN(c2nc(OC[C@@]34CCCN3C[C@H](F)C4)nc3sc4c(-c5c(C(F)(F)F)c(Cl)cc6[nH]ncc56)nccc4c23)C1] | (6S)-4-(8-(6-chloro-5- (trifluoromethyl)-1H- indazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 692 |
| Ex- 41 | 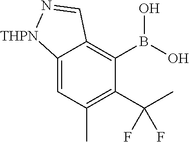 Int-40 | 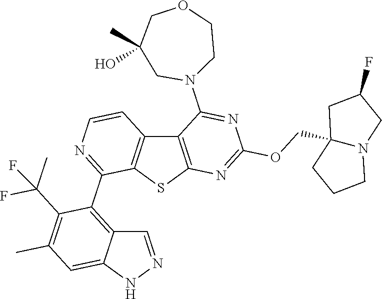 [SMILES: Cc1cc2[nH]ncc2c(-c2nccc3c2sc2nc(OC[C@@]45CCCN4C[C@H](F)C5)nc(N4CCOC[C@@](C)(O)C4)c23)c1C(C)(F)F] | (6S)-4-(8-(5-(1,1- difluoroethyl)-6-methyl- 1H-indazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 668 |
| Ex- 42 | 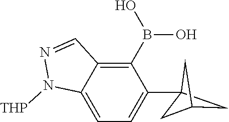 Int-47 | 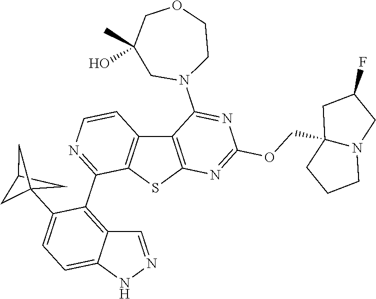 [SMILES: C[C@@]1(O)COCCN(c2nc(OC[C@@]34CCCN3C[C@H](F)C4)nc3sc4c(-c5c(C67CC(C6)C7)ccc6[nH]ncc56)nccc4c23)C1] | (6S)-4-(8-(5- (bicyclo[1.1.1]pentan-1- yl)-1H-indazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 656 |
| Ex- 43 | 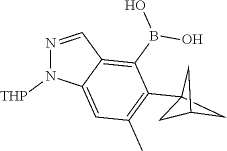 Int-48 | 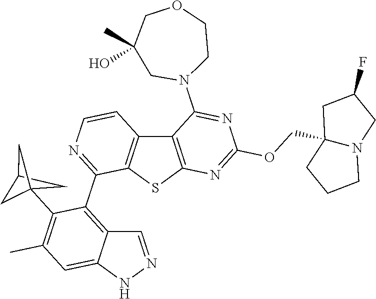 [SMILES: Cc1cc2[nH]ncc2c(-c2nccc3c2sc2nc(OC[C@@]45CCCN4C[C@H](F)C5)nc(N4CCOC[C@@](C)(O)C4)c23)c1C12CC(C1)C2] | (6S)-4-(8-(5- (bicyclo[1.1.1]pentan-1 yl)-6-methy1-1H- indazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 670 |
| Ex- 44 | 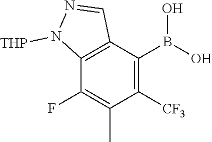 Int-3 | 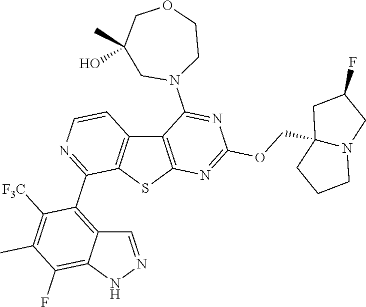 [SMILES: Cc1c(C(F)(F)F)c(-c2nccc3c2sc2nc(OC[C@@]45CCCN4C[C@H](F)C5)nc(N4CCOC[C@@](C)(O)C4)c23)c2cn[nH]c2c1F] | (6S)-4-(8-(7-fluoro-6- methyl-5- (trifluoromethyl)-1H- indazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 690 |
| Ex- 45 | 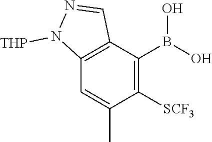 Int-14 | 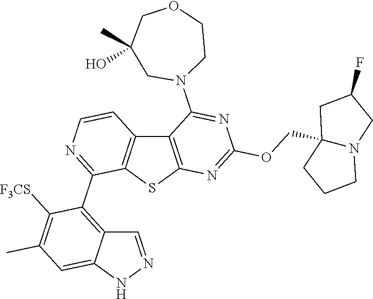 | (6S)-4-(2-(((2R,7aS)-2- fluorotetrahydro-1H- pyrrolizin-7a(5H)- yl)methoxy)-8-(6- methyl-5- ((trifluoromethyl)thio)- 1H-indazol-4- yl)pyrido[4′,3′:4,5]thieno [2,3-d]pyrimidin-4-yl)- 6-methyl-1,4-oxazepan- 6-ol | | 704 |
| Ex- 46 | 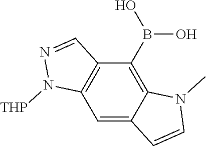 Int-66 | 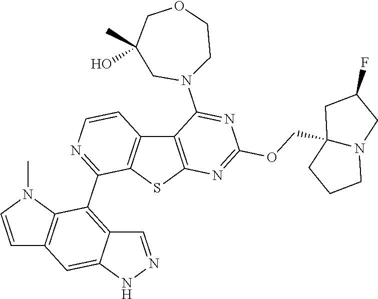 [SMILES: Cn1ccc2cc3[nH]ncc3c(-c3nccc4c3sc3nc(OC[C@@]56CCCN5C[C@H](F)C6)nc(N5CCOC[C@@](C)(O)C5)c34)c21] | (6S)-4-(2-(((2R,7aS)-2- fluorotetrahydro-1H- pyrrolizin-7a(5H)- yl)methoxy)-8-(5- methyl-1,5- dihydropyrrolo[2,3- f]indazol-4- yl)pyrido[4′,3′:4,5]thieno [2,3-d]pyrimidin-4-y])- 6-methyl-1,4-oxazepan- 6-ol | | 643 |
| Ex- 47 | 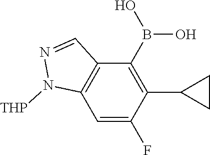 Int-38 | 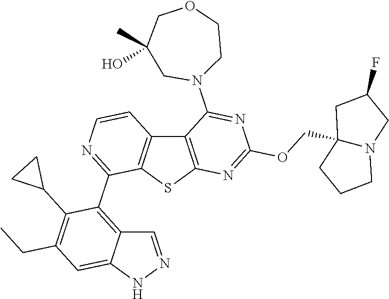 [SMILES: CCc1cc2[nH]ncc2c(-c2nccc3c2sc2nc(OC[C@@]45CCCN4C[C@H](F)C5)nc(N4CCOC[C@@](C)(O)C4)c23)c1C1CC1] | (6S)-4-(8-(5- cyclopropyl-6-fluoro- 1H-indazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 648 |
| Ex- 48 | 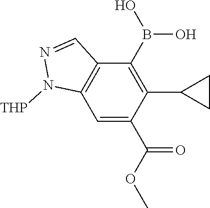 Int-70 | 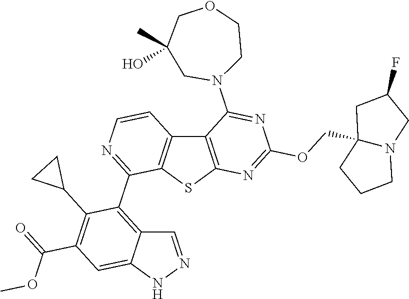 [SMILES: COC(=O)c1cc2[nH]ncc2c(-c2nccc3c2sc2nc(OC[C@@]45CCCN4C[C@H](F)C5)nc(N4CCOC[C@@](C)(O)C4)c23)c1C1CC1] | (6S)-4-(8-(5- cyclopropyl-6-ethyl-1H- indazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 658 |
| Ex- 49 | 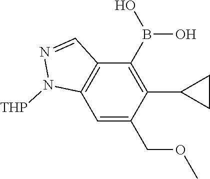 Int-68 | 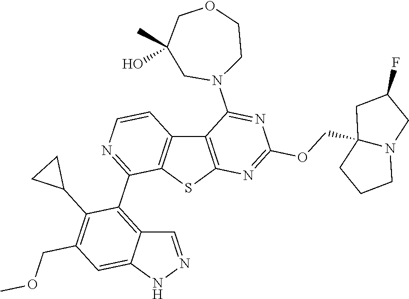 [SMILES: COCc1cc2[nH]ncc2c(-c2nccc3c2sc2nc(OC[C@@]45CCCN4C[C@H](F)C5)nc(N4CCOC[C@@](C)(O)C4)c23)c1C1CC1] | Methyl 5-cyclopropyl- 4-(2-(((2R,7aS)-2- fluorotetrahydro-1H- pyrrolizin-7a(5H)- yl)methoxy)-4-((S)-6- hydroxy-6-methyl-1,4- oxazepan-4- yl)pyrido[4′,3′:4,5]thieno [2,3-d]pyrimidin-8-yl)- 1H-indazole-6- carboxylate | | 688 |
| Ex- 50 | 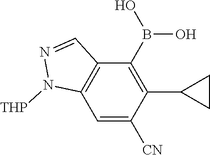 Int-69 | 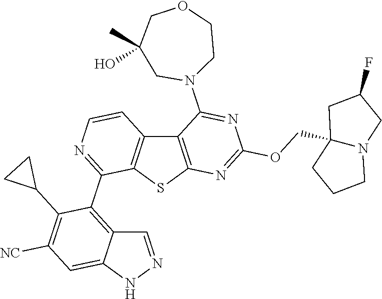 [SMILES: C[C@@]1(O)COCCN(c2nc(OC[C@@]34CCCN3C[C@H](F)C4)nc3sc4c(-c5c(C6CC6)c(C#N)cc6[nH]ncc56)nccc4c23)C1] | (6S)-4-(8-(5- cyclopropyl-6- (methoxymethyl)-1H- indazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 674 |
| Ex- 51 | 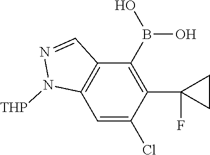 Int-71 | 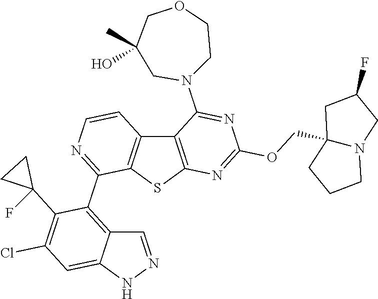 [SMILES: C[C@@]1(O)COCCN(c2nc(OC[C@@]34CCCN3C[C@H](F)C4)nc3sc4c(-c5c(C6(F)CC6)c(Cl)cc6[nH]ncc56)nccc4c23)C1] | 5-cyclopropy1-4-(2- (((2R,7aS)-2- fluorotetrahydro-1H- pyrrolizin-7a(5H)- yl)methoxy)-4-((S)-6- hydroxy-6-methyl-1,4- oxazepan-4- yl)pyrido[4′,3′:4,5]thieno [2,3-d]pyrimidin-8-yl)- 1H-indazole-6- carbonitrile | | 655 |
| Ex- 52 | 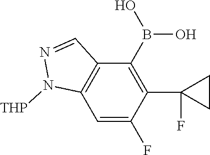 Int-44 | 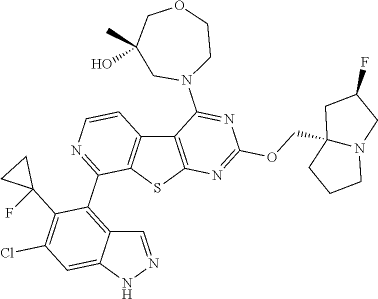 [SMILES: C[C@@]1(O)COCCN(c2nc(OC[C@@]34CCCN3C[C@H](F)C4)nc3sc4c(-c5c(C6(F)CC6)c(Cl)cc6[nH]ncc56)nccc4c23)C1] | (6S)-4-(8-(6-chloro-5- (1-fluorocyclopropyl)- 1H-indazol-4-yl)-2- (((2R,7aS)-2- fluorotetrahydro-1H- pyrrolizin-7a(5H)- yl)methoxy)pyrido[4′,3′-4,5]thi- eno[2,3- d]pyrimidin-4-yl)-6- methyl-1,4-oxazepan-6- ol | | 682 |
| Ex- 53 | 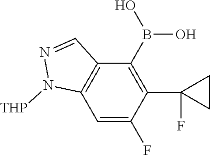 Int-17 | 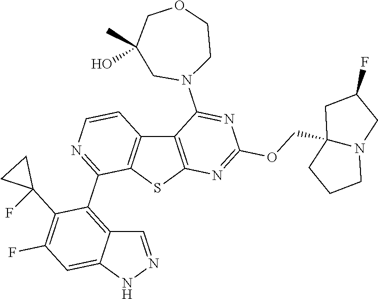 [SMILES: C[C@@]1(O)COCCN(c2nc(OC[C@@]34CCCN3C[C@H](F)C4)nc3sc4c(-c5c(C6(F)CC6)c(F)cc6[nH]ncc56)nccc4c23)C1] | (6S)-4-(8-(6-fluoro-5- (1-fluorocyclopropyl)- 1H-indazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 666 |
| Ex- 54 | 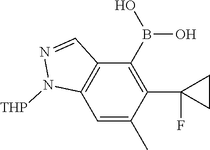 Int-45 | 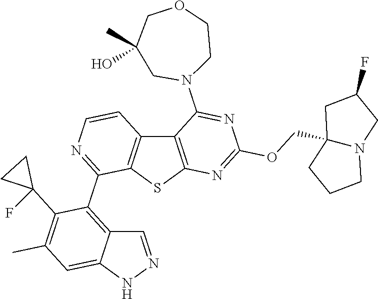 [SMILES: Cc1cc2[nH]ncc2c(-c2nccc3c2sc2nc(OC[C@@]45CCCN4C[C@H](F)C5)nc(N4CCOC[C@@](C)(O)C4)c23)c1C1(F)CC1] | (6S)-4-(8-(5-(1- fluorocyclopropyl)-6- methyl-1H-indazol-4- yl)-2-(((2R,7aS)-2- fluorotetrahydro-1H- pyrrolizin-7a(5H)- yl)methoxy)pyrido[4′,3′:4,5]thi- eno[2,3- d)pyrimidin-4-yl)-6- methyl-1,4-oxazepan-6- ol | | 662 |
| Ex- 55 | 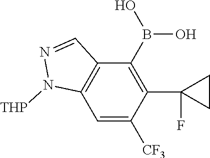 Int-49 | 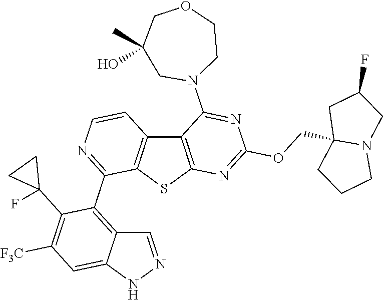 [SMILES: C[C@@]1(O)COCCN(c2nc(OC[C@@]34CCCN3C[C@H](F)C4)nc3sc4c(-c5c(C6(F)CC6)c(C(F)(F)F)cc6[nH]ncc56)nccc4c23)C1] | (6S)-4-(8-(5-(1- fluorocyclopropyl)-6- (trifluoromethyl)-1H- indazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 716 |
| Ex- 56 | 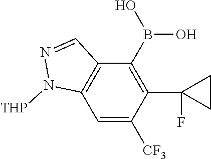 Int-49 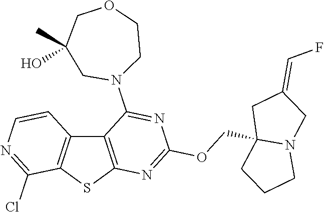 [SMILES: C[C@@]1(O)COCCN(c2nc(OC[C@@]34CCCN3C/C(=C\F)C4)nc3sc4c(Cl)nccc4c23)C1] | 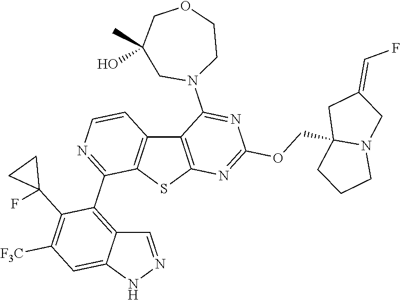 [SMILES: C[C@@]1(O)COCCN(c2nc(OC[C@@]34CCCN3C/C(=C\F)C4)nc3sc4c(-c5c(C6(F)CC6)c(C(F)(F)F)cc6[nH]ncc56)nccc4c23)C1] | (6S)-4-(8-(5-(1- fluorocyclopropyl)-6- (trifluoromethyl)-1H- indazol-4-yl)-2-(((S,Z)- 2- (fluoromethylene)tetrahy- dro-1H-pyrrolizin- 7a(5H)- yl)methoxy)pyrido[4′,3′:4,5]thi- eno[2,3- d]pyrimidin-4-yl)-6- methyl-1,4-oxazepan-6- ol | | 728 |
| | Int-145 | | | | |
| Ex- 57 | 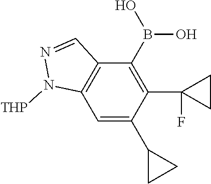 Int-55 | 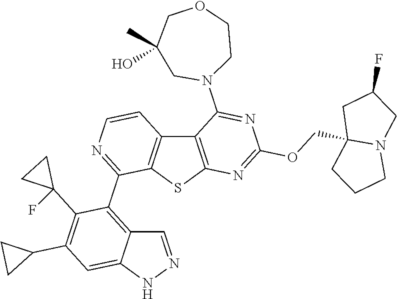 [SMILES: C[C@@]1(O)COCCN(c2nc(OC[C@@]34CCCN3C[C@H](F)C4)nc3sc4c(-c5c(C6(F)CC6)c(C6CC6)cc6[nH]ncc56)nccc4c23)C1] | (6S)-4-(8-(6- cyclopropyl-5-(1- fluorocyclopropyl)-1H- indazol-4-yl)-2- (((2R,7aS)-2- fluorotetrahydro-1H- pyrrolizin-7a(5H)- yl)methoxy)pyrido[4′,3′: 4,5]thieno[2,3- d]pyrimidin-4-yl)-6- methyl-1,4-oxazepan-6- ol | | 688 |
| Ex- 58 | 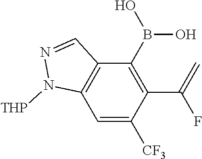 Int-51 | 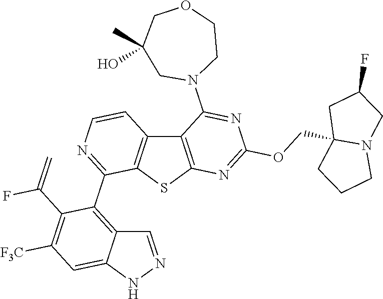 [SMILES: C=C(F)c1c(C(F)(F)F)cc2[nH]ncc2c1-c1nccc2c1sc1nc(OC[C@@]34CCCN3C[C@H](F)C4)nc(N3CCOC[C@@](C)(O)C3)c12] | (6S)-4-(2-(((2R,7aS)-2- fluorotetrahydro-1H- pyrrolizin-7a(5H)- yl)methoxy )-8-(5-(1- fluorovinyl)-6- (trifluoromethyl)-1H- indazol-4- yl)pyrido[4′,3′:4,5]thieno [2,3-d]pyrimidin-4-yl)- 6-methyl-1,4-oxazepan- 6-ol | | 702 |
| Ex- 59 | Byproduct of Ex-58 | 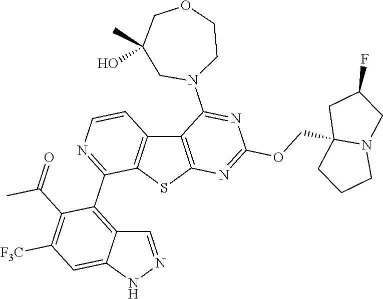 [SMILES: CC(=O)c1c(C(F)(F)F)cc2[nH]ncc2c1-c1nccc2c1sc1nc(OC[C@@]34CCCN3C[C@H](F)C4)nc(N3CCOC[C@@](C)(O)C3)c12] | 1-(4-(2-(((2R,7aS)-2- fluorotetrahydro-1H- pyrrolizin-7a(5H)- yl)methoxy)-4-((S)-6- hydroxy-6-methyl-1,4- oxazepan-4- yl)pyrido[4′,3′:4,5]thieno [2,3-d]pyrimidin-8-y])- 6-(trifluoromethyl)-1H- indazol-5-yl)ethan-1- one | | 700 |
| Ex- 60 | 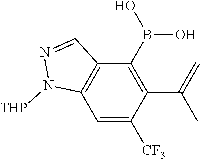 Int-11 | 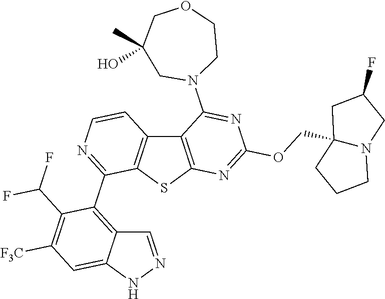 [SMILES: C[C@@]1(O)COCCN(c2nc(OC[C@@]34CCCN3C[C@H](F)C4)nc3sc4c(-c5c(C(F)F)c(C(F)(F)F)cc6[nH]ncc56)nccc4c23)C1] | (6S)-4-(2-(((2R,7aS)-2- fluorotetrahydro-1H- pyrrolizin-7a(5H)- yl)methoxy)-8-(5-(prop- 1-en-2-yl)-6- (trifluoromethyl)-1H- indazol-4- yl)pyrido[4′,3′:4,5]thieno [2,3-d]pyrimidin-4-y])- 6-methyl-1,4-oxazepan- 6-ol | | 698 |
| Ex- 61 | 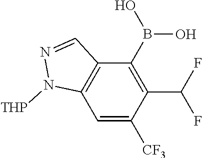 Int-52 | 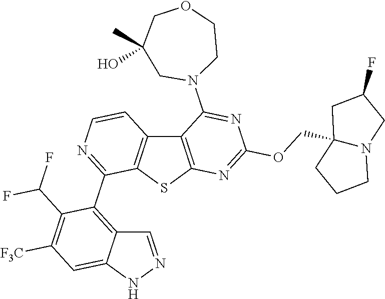 [SMILES: C[C@@]1(O)COCCN(c2nc(OC[C@@]34CCCN3C[C@H](F)C4)nc3sc4c(-c5c(C(F)F)c(C(F)(F)F)cc6[nH]ncc56)nccc4c23)C1] | (6S)-4-(8-(5- (difluoromethyl)-6- (trifluoromethyl)-1H- indazol-4-yl)-2- (((2R,7aS)-2- fluorotetrahydro-1H- pyrrolizin-7a(5H)- yl)methoxy )pyrido[4′,3′:4,5]thi- eno[2,3- d]pyrimidin-4-yl)-6- methyl-1,4-oxazepan-6- ol | | 708 |
| Ex- 62 | 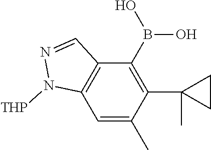 Int-29 | 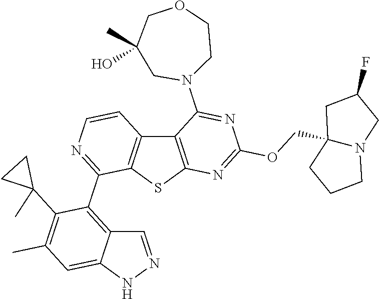 [SMILES: Cc1cc2[nH]ncc2c(-c2nccc3c2sc2nc(OC[C@@]45CCCN4C[C@H](F)C5)nc(N4CCOC[C@@](C)(O)C4)c23)c1C1(C)CC1] | (6S)-4-(2-(((2R,7aS)-2- fluorotetrahydro-1H- pyrrolizin-7a(5H)- yl)methoxy)-8-(6- methyl-5-(1- methylcyclopropyl)-1H- indazol-4- yl)pyrido[4′,3′:4,5]thieno [2,3-d]pyrimidin-4-yl)- 6-methyl-1,4-oxazepan- 6-ol | | 658 |
| Ex- 63 | 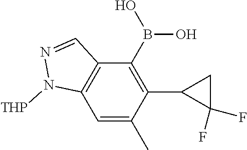 Int-7 | 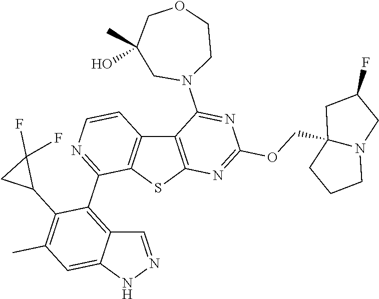 [SMILES: Cc1cc2[nH]ncc2c(-c2nccc3c2sc2nc(OC[C@@]45CCCN4C[C@H](F)C5)nc(N4CCOC[C@@](C)(O)C4)c23)c1C1CC1(F)F] | (6S)-4-(8-(5-(2,2- difluorocyclopropyl)-6- methyl-1H-indazol-4- yl)-2-(((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 680 |
| Ex- 64 | 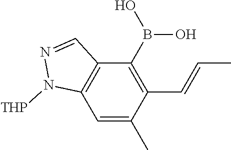 Int-5 | 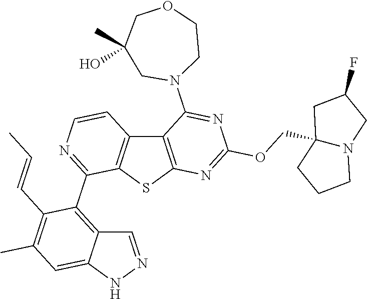 [SMILES: C/C=C/c1c(C)cc2[nH]ncc2c1-c1nccc2c1sc1nc(OC[C@@]34CCCN3C[C@H](F)C4)nc(N3CCOC[C@@](C)(O)C3)c12] | (6S)-4-(2-(((2R,7aS)-2- fluorotetrahydro-1H- pyrrolizin-7a(5H)- yl)methoxy)-8-(6- methyl-5-((E)-prop-1- en-1-yl)-1H-indazol-4- yl)pyrido[4′,3′:4,5]thieno [2,3-d]pyrimidin-4-yl)- 6-methyl-1,4-oxazepan- 6-ol | | 644 |
| Ex- 65 | 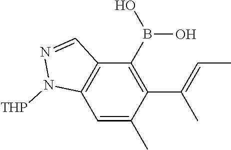 Int-6 | 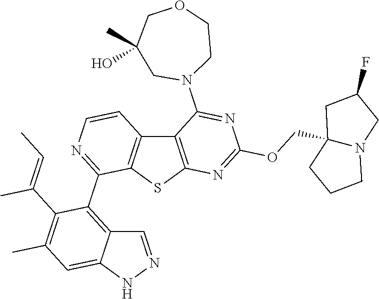 [SMILES: C/C=C(\C)c1c(C)cc2[nH]ncc2c1-c1nccc2c1sc1nc(OC[C@@]34CCCN3C[C@H](F)C4)nc(N3CCOC[C@@](C)(O)C3)c12] | (6S)-4-(8-(5-((E)-but-2- en-2-yl)-6-methyl-1H- indazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 658 |
| Ex- 66 | 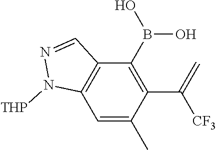 Int-20 | 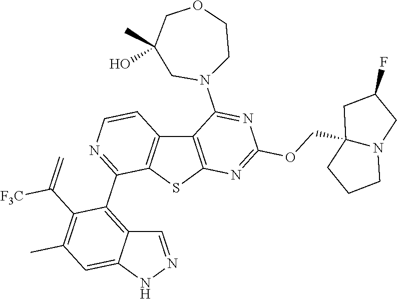 [SMILES: C=C(c1c(C)cc2[nH]ncc2c1-c1nccc2c1sc1nc(OC[C@@]34CCCN3C[C@H](F)C4)nc(N3CCOC[C@@](C)(O)C3)c12)C(F)(F)F] | (6S)-4-(2-(((2R,7aS)-2- fluorotetrahydro-1H- pyrrolizin-7a(5H)- yl)methoxy)-8-(6- methyl-5-(3,3,3- trifluoroprop-1-en-2- yl)-1H-indazol-4- yl)pyrido[4′,3′:4,5]thieno [2,3-d]pyrimidin-4-yl)- 6-methyl-1,4-oxazepan- 6-ol | | 698 |
| Ex- 67 | 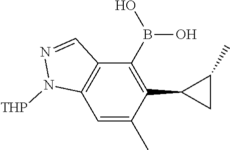 Int-30 | 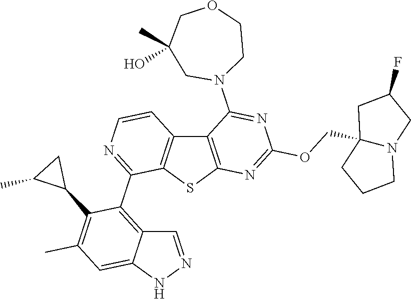 [SMILES: Cc1cc2[nH]ncc2c(-c2nccc3c2sc2nc(OC[C@@]45CCCN4C[C@H](F)C5)nc(N4CCOC[C@@](C)(O)C4)c23)c1[C@@H]1C[C@H]1C] | (S)-4-((R)-2-(((2R,7aS)- 2-fluorotetrahydro-1H- pyrrolizin-7a(5H)- yl)methoxy)-8-(6- methyl-5-((1R,2R)-2- methylcyclopropyl)-1H- indazol-4- yl)pyrido[4′,3′:4,5]thieno [2,3-d]pyrimidin-4-yl)- 6-methyl-1,4-oxazepan- 6-o1 | | 658 |
| Ex- 68 | 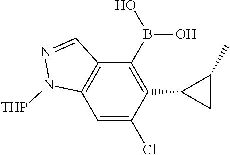 Int-31 | 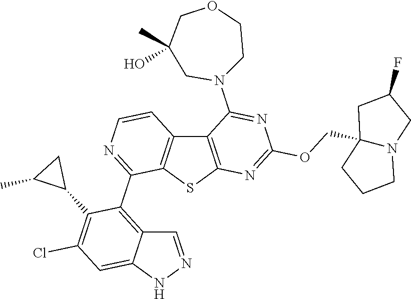 [SMILES: C[C@@H]1C[C@@H]1c1c(Cl)cc2[nH]ncc2c1-c1nccc2c1sc1nc(OC[C@@]34CCCN3C[C@H](F)C4)nc(N3CCOC[C@@](C)(O)C3)c12] | (S)-4-((R)-8-(6-chloro- 5-((1S,2R)-2- methylcyclopropyl)-1H- indazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 678 |
| Ex- 69 | 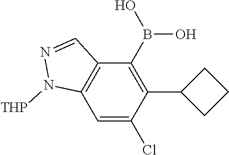 Int-42 | 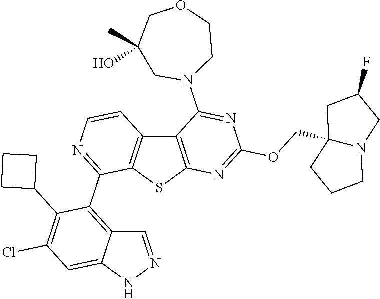 [SMILES: C[C@@]1(O)COCCN(c2nc(OC[C@@]34CCCN3C[C@H](F)C4)nc3sc4c(-c5c(C6CCC6)c(Cl)cc6[nH]ncc56)nccc4c23)C1] | (6S)-4-(8-(6-chloro-5- cyclobutyl-1H-indazol- 4-yl)-2-(((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 678 |
| Ex- 70 | 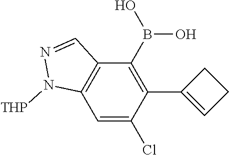 Int-43 | 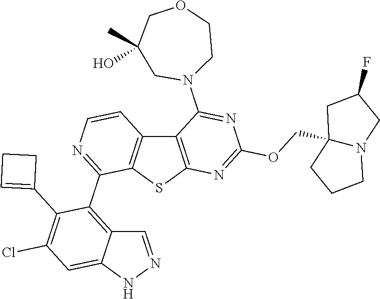 [SMILES: C[C@@]1(O)COCCN(c2nc(OC[C@@]34CCCN3C[C@H](F)C4)nc3sc4c(-c5c(C6=CCC6)c(Cl)cc6[nH]ncc56)nccc4c23)C1] | (6S)-4-(8-(6-chloro-5- (cyclobut-1-en-1-yl)- 1H-indazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 676 |
| Ex- 71 | 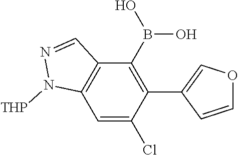 Int-23 | 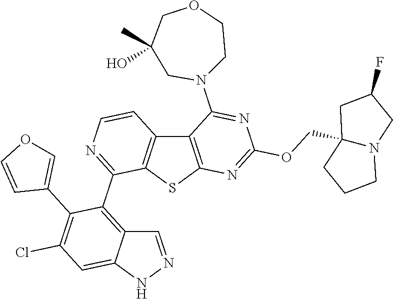 [SMILES: C[C@@]1(O)COCCN(c2nc(OC[C@@]34CCCN3C[C@H](F)C4)nc3sc4c(-c5c(-c6ccoc6)c(Cl)cc6[nH]ncc56)nccc4c23)C1] | (6S)-4-(8-(6-chloro-5- (furan-3-yl)-1H- indazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 690 |
| Ex- 72 | 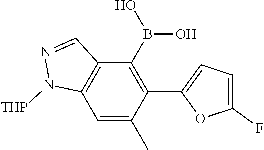 Int-36 | 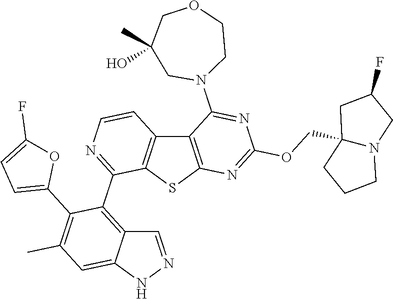 [SMILES: Cc1cc2[nH]ncc2c(-c2nccc3c2sc2nc(OC[C@@]45CCCN4C[C@H](F)C5)nc(N4CCOC[C@@](C)(O)C4)c23)c1-c1ccc(F)o1] | (6S)-4-(8-(5-(5- fluorofuran-2-yl)-6- methyl-1H-indazol-4- yl)-2-(((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 688 |
| Ex- 73 | 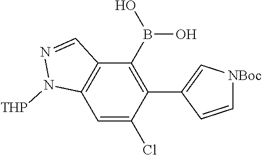 Int-24 | 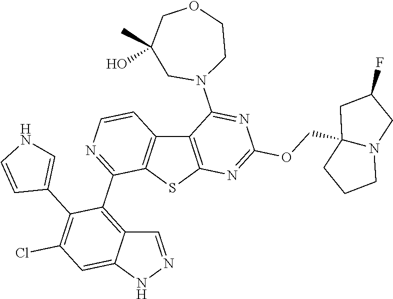 [SMILES: C[C@@]1(O)COCCN(c2nc(OC[C@@]34CCCN3C[C@H](F)C4)nc3sc4c(-c5c(-c6cc[nH]c6)c(Cl)cc6[nH]ncc56)nccc4c23)C1] | (6S)-4-(8-(6-chloro-5- (1H-pyrrol-3-yl)-1H- indazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 689 |
| Ex- 74 | 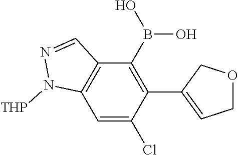 Int-32 | 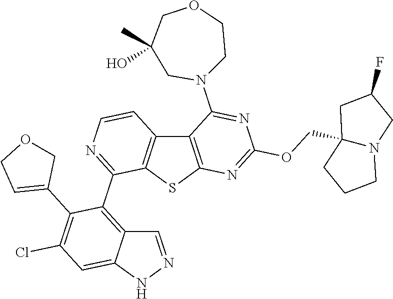 [SMILES: C[C@@]1(O)COCCN(c2nc(OC[C@@]34CCCN3C[C@H](F)C4)nc3sc4c(-c5c(C6=CCOC6)c(Cl)cc6[nH]ncc56)nccc4c23)C1] | (6S)-4-(8-(6-chloro-5- (2,5-dihydrofuran-3-yl)- 1H-indazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 692 |
| Ex- 75 | 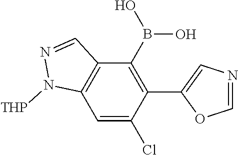 Int-25 | 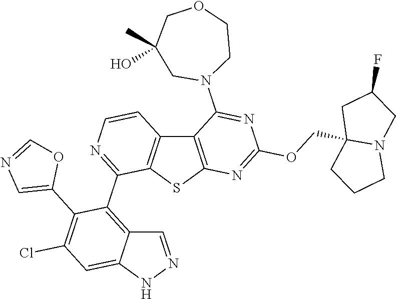 [SMILES: C[C@@]1(O)COCCN(c2nc(OC[C@@]34CCCN3C[C@H](F)C4)nc3sc4c(-c5c(-c6cnco6)c(Cl)cc6[nH]ncc56)nccc4c23)C1] | (6S)-4-(8-(6-chloro-5- (oxazol-5-yl)-1H- indazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 691 |
| Ex- 76 | 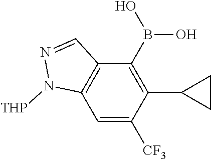 Int-50 | 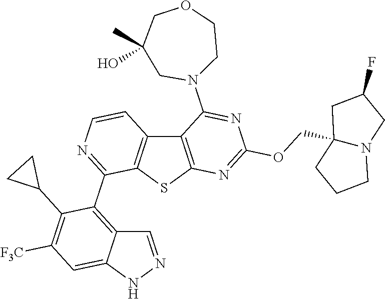 [SMILES: C[C@@]1(O)COCCN(c2nc(OC[C@@]34CCCN3C[C@H](F)C4)nc3sc4c(-c5c(C6CC6)c(C(F)(F)F)cc6[nH]ncc56)nccc4c23)C1] | (6S)-4-(8-(5- cyclopropyl-6- (trifluoromethyl)-1H- indazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 698 |
| Ex- 77 | 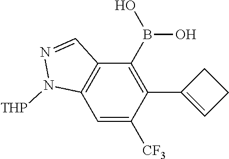 Int-54 | 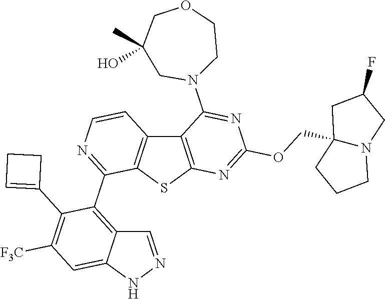 [SMILES: C[C@@]1(O)COCCN(c2nc(OC[C@@]34CCCN3C[C@H](F)C4)nc3sc4c(-c5c(C6=CCC6)c(C(F)(F)F)cc6[nH]ncc56)nccc4c23)C1] | (6S)-4-(8-(5-(cyclobut- 1-en-1-yl)-6- (trifluoromethyl)-1H- indazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 710 |
| Ex- 78 | 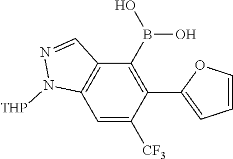 Int-53 | 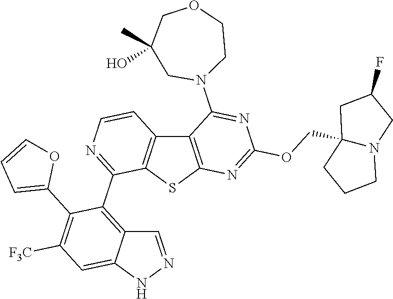 [SMILES: C[C@@]1(O)COCCN(c2nc(OC[C@@]34CCCN3C[C@H](F)C4)nc3sc4c(-c5c(-c6ccco6)c(C(F)(F)F)cc6[nH]ncc56)nccc4c23)C1] | (6S)-4-(2-(((2R,7aS)-2- fluorotetrahydro-1H- pyrrolizin-7a(5H)- yl)methoxy)-8-(5- (furan-2-yl)-6- (trifluoromethyl)-1H- indazol-4- yl)pyrido[4′,3′:4,5]thieno [2,3-d]pyrimidin-4-y])- 6-methy1-1,4-oxazepan- 6-ol | | 724 |
| Ex- 79 | 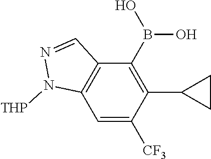 Int-50 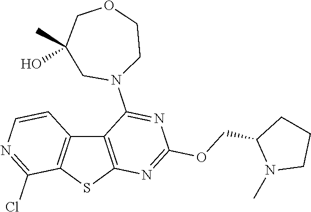 [SMILES: CN1CCC[C@H]1COc1nc(N2CCOC[C@@](C)(O)C2)c2c(n1)sc1c(Cl)nccc12] | 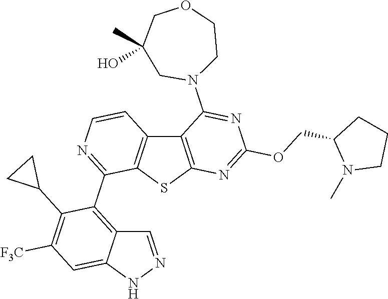 [SMILES: CN1CCC[C@H]1COc1nc(N2CCOC[C@@](C)(O)C2)c2c(n1)sc1c(-c3c(C4CC4)c(C(F)(F)F)cc4[nH]ncc34)nccc12] | (6S)-4-(8-(5- cyclopropyl-6- (trifluoromethyl)-1H- indazol-4-yl)-2-(((S)-1- methylpyrrolidin-2- yl)methoxy)pyrido[4′,3′:4.5]thi- eno[2,3- d]pyrimidin-4-yl)-6- methyl-1,4-oxazepan-6- ol | | 654 |
| | Int-51 | | | | |
| Ex- 80 | 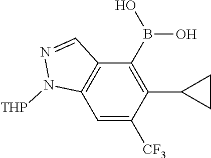 Int-33 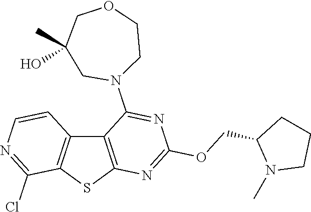 [SMILES: CN1CCC[C@H]1COc1nc(N2CCOC[C@@](C)(O)C2)c2c(n1)sc1c(Cl)nccc12] | 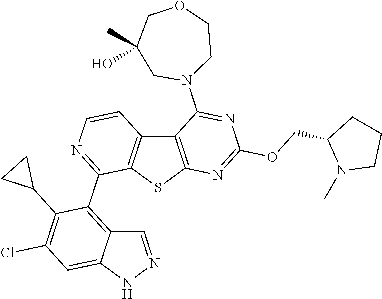 [SMILES: CN1CCC[C@H]1COc1nc(N2CCOC[C@@](C)(O)C2)c2c(n1)sc1c(-c3c(C4CC4)c(Cl)cc4[nH]ncc34)nccc12] | (6S)-4-(8-(6-chloro-5- cyclopropyl-1H- indazol-4-yl)-2-(((S)-1- methylpyrrolidin-2- yl)methoxy)pyrido[4′,3′:4,5]thi- eno[2,3- d]pyrimidin-4-yl)-6- methy1-1,4-oxazepan-6- ol | | 620 |
| | Int-151 | | | | |
| Ex- 81 | 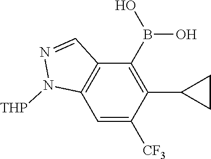 Int-50 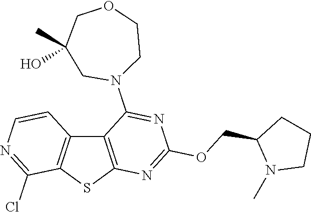 [SMILES: CN1CCC[C@@H]1COc1nc(N2CCOC[C@@](C)(O)C2)c2c(n1)sc1c(Cl)nccc12] | 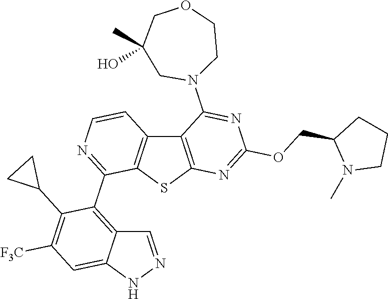 [SMILES: CN1CCC[C@@H]1COc1nc(N2CCOC[C@@](C)(O)C2)c2c(n1)sc1c(-c3c(C4CC4)c(C(F)(F)F)cc4[nH]ncc34)nccc12] | (6S)-4-(8-(5- cyclopropyl-6- (trifluoromethyl)-1H- indazol-4-yl)-2-(((R)-1- methylpyrrolidin-2- yl)methoxy)pyrido[4′,3′:4,5]thi- eno[2,3- d]pyrimidin-4-yl)-6- methyl-1,4-oxazepan-6- ol | | 654 |
| | Int-152 | | | | |
| Ex- 82 | 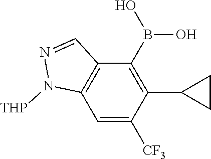 Int-50 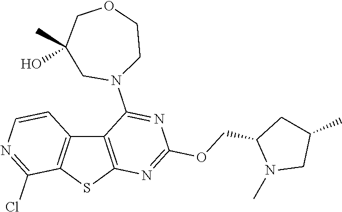 [SMILES: C[C@H]1C[C@@H](COc2nc(N3CCOC[C@@](C)(O)C3)c3c(n2)sc2c(Cl)nccc23)N(C)C1] | 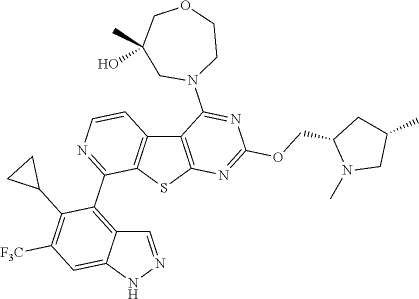 [SMILES: C[C@H]1C[C@@H](COc2nc(N3CCOC[C@@](C)(O)C3)c3c(n2)sc2c(-c4c(C5CC5)c(C(F)(F)F)cc5[nH]ncc45)nccc23)N(C)C1] | (6S)-4-(8-(5- cyclopropyl-6- (trifluoromethyl)-1H- indazol-4-yl)-2- (((2S,4S)-1,4- dimethylpyrrolidin-2- yl)methoxy)pyrido[4′,3′:4,5]thi- eno[2,3- d]pyrimidin-4-yl)-6- methyl-1,4-oxazepan-6- ol | | 668 |
| | Int-148 | | | | |
| Ex- 83 | 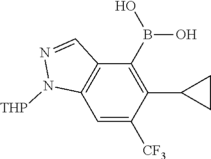 Int-50 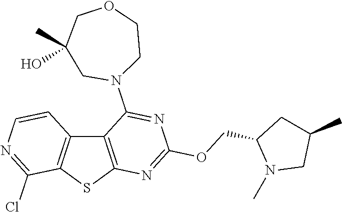 [SMILES: C[C@@H]1C[C@@H](COc2nc(N3CCOC[C@@](C)(O)C3)c3c(n2)sc2c(Cl)nccc23)N(C)C1] | 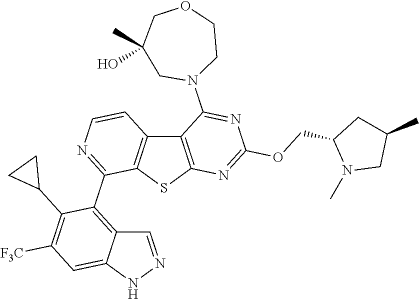 [SMILES: C[C@@H]1C[C@@H](COc2nc(N3CCOC[C@@](C)(O)C3)c3c(n2)sc2c(-c4c(C5CC5)c(C(F)(F)F)cc5[nH]ncc45)nccc23)N(C)C1] | (6S)-4-(8-(5- cyclopropyl-6- (trifluoromethyl)-1H- indazol-4-yl)-2- (((2S,4R)-1,4- dimethylpyrrolidin-2- yl)methoxy)pyrido[4′,3′:4,5]thi- eno[2,3- d]pyrimidin-4-yl)-6- methyl-1,4-oxazepan-6- ol | | 668 |
| | Int-149 | | | | |
| Ex- 84 | 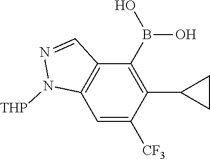 Int-50 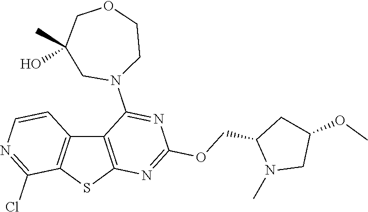 [SMILES: CO[C@H]1C[C@@H](COc2nc(N3CCOC[C@@](C)(O)C3)c3c(n2)sc2c(Cl)nccc23)N(C)C1] | 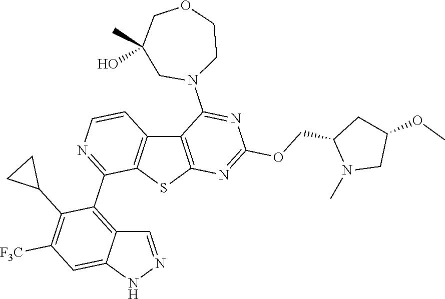 [SMILES: CO[C@H]1C[C@@H](COc2nc(N3CCOC[C@@](C)(O)C3)c3c(n2)sc2c(-c4c(C5CC5)c(C(F)(F)F)cc5[nH]ncc45)nccc23)N(C)C1] | (6S)-4-(8-(5- cyclopropyl-6- (trifluoromethyl)-1H- indazol-4-yl)-2- (((2S,4S)-4-methoxy-1- methylpyrrolidin-2- yl)methoxy)pyrido[4′,3′:4,5]thi- eno[2,3- d]pyrimidin-4-yl)-6- methyl-1,4-oxazepan-6- ol | | 684 |
| | Int-146 | | | | |
| Ex- 85 | 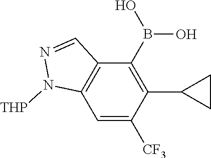 Int-50 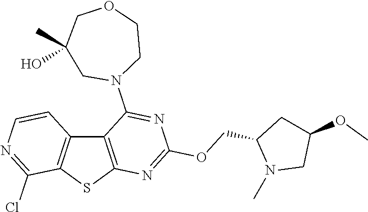 [SMILES: CO[C@@H]1C[C@@H](COc2nc(N3CCOC[C@@](C)(O)C3)c3c(n2)sc2c(Cl)nccc23)N(C)C1] | 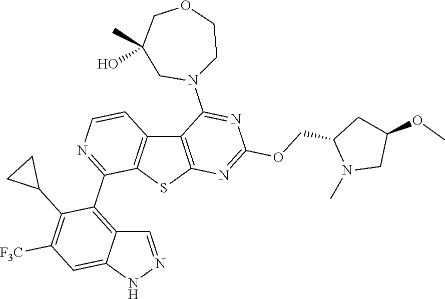 [SMILES: CO[C@@H]1C[C@@H](COc2nc(N3CCOC[C@@](C)(O)C3)c3c(n2)sc2c(-c4c(C5CC5)c(C(F)(F)F)cc5[nH]ncc45)nccc23)N(C)C1] | (6S)-4-(8-(5- cyclopropyl-6- (trifluoromethyl)-1H- indazol-4-yl)-2- (((2S,4R)-4-methoxy-1- methylpyrrolidin-2- yl)methoxy)pyrido[4′,3′:4,5]thi- eno[2,3- d]pyrimidin-4-yl)-6- methyl-1,4-oxazepan-6- ol | | 684 |
| | Int-147 | | | | |
| Ex- 86 | 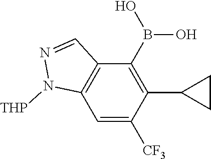 Int-50 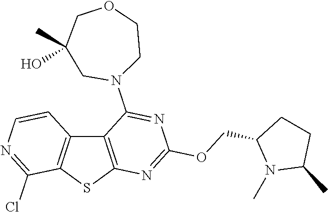 [SMILES: C[C@@H]1CC[C@@H](COc2nc(N3CCOC[C@@](C)(O)C3)c3c(n2)sc2c(Cl)nccc23)N1C] | 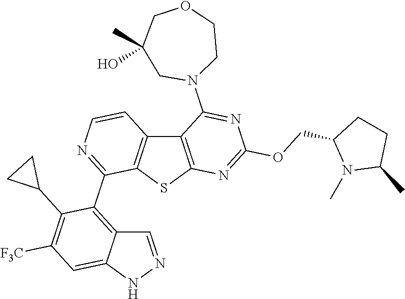 [SMILES: C[C@@H]1CC[C@@H](COc2nc(N3CCOC[C@@](C)(O)C3)c3c(n2)sc2c(-c4c(C5CC5)c(C(F)(F)F)cc5[nH]ncc45)nccc23)N1C] | (6S)-4-(8-(5- cyclopropyl-6- (trifluoromethyl)-1H- indazol-4-yl)-2- (((2S,5R)-1,5- dimethylpyrrolidin-2- yl)methoxy)pyrido[4′,3′:4,5]thi- eno[2,3- d]pyrimidin-4-yl)-6- methyl-1,4-oxazepan-6- ol | | 668 |
| | Int-150 | | | | |
| Ex- 87 | 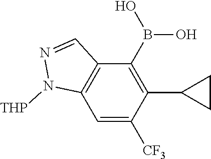 Int-50 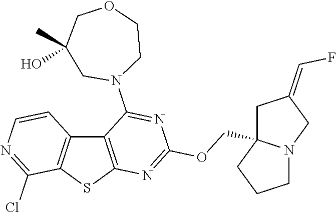 [SMILES: C[C@@]1(O)COCCN(c2nc(OC[C@@]34CCCN3C/C(=C\F)C4)nc3sc4c(Cl)nccc4c23)C1] | 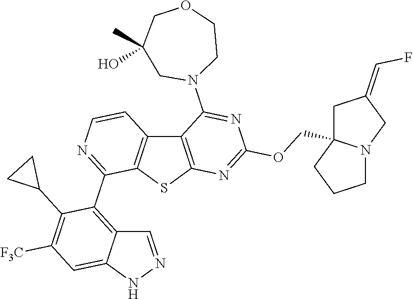 [SMILES: C[C@@]1(O)COCCN(c2nc(OC[C@@]34CCCN3C/C(=C\F)C4)nc3sc4c(-c5c(C6CC6)c(C(F)(F)F)cc6[nH]ncc56)nccc4c23)C1] | (6S)-4-(8-(5- cyclopropyl-6- (trifluoromethyl)-1H- indazol-4-yl)-2-(((S,Z)- 2- (fluoromethylene)tetrahy- dro-1H-pyrrolizin- 7a(5H)- yl)methoxy)pyrido[4′,3′:4,5]thi- eno[2,3- d]pyrimidin-4-yl)-6- methyl-1,4-oxazepan-6- ol | | 710 |
| | Int-145 | | | | |
| Ex- 88 | 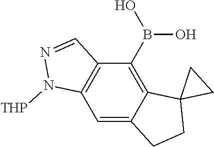 Int-74 | 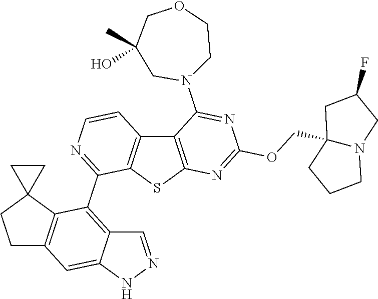 [SMILES: C[C@@]1(O)COCCN(c2nc(OC[C@@]34CCCN3C[C@H](F)C4)nc3sc4c(-c5c6c(cc7[nH]ncc57)CCC65CC5)nccc4c23)C1] | (6S)-4-(8-(6,7-dihydro- 1H- spiro[cyclopenta[f]indazole- 5,1′-cyclopropan]-4- yl)-2-(((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 656 |
| Ex- 89 | 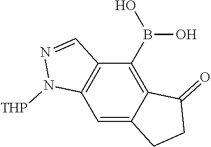 Int-82 | 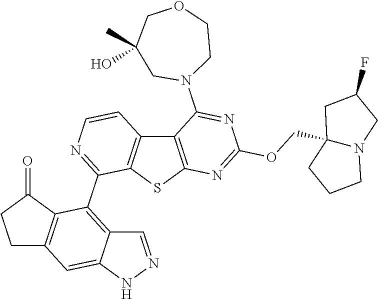 [SMILES: C[C@@]1(O)COCCN(c2nc(OC[C@@]34CCCN3C[C@H](F)C4)nc3sc4c(-c5c6c(cc7[nH]ncc57)CCC6=O)nccc4c23)C1] | 4-(2-(((2R,7aS)-2- fluorotetrahydro-1H- pyrrolizin-7a(5H)- yl)methoxy)-4-((S)-6- hydroxy-6-methyl-1,4- oxazepan-4- yl)pyrido[4′,3′:4,5]thieno [2,3-d]pyrimidin-8-yl)- 6,7- dihydrocyclopenta[f]inda- zol-5(1H)-one | | 644 |
| Ex- 90 | 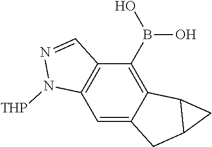 Int-75 | 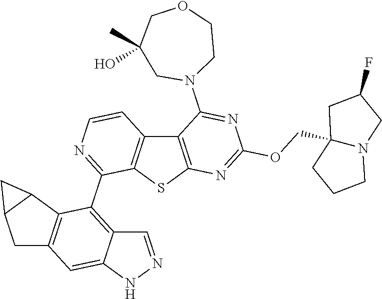 [SMILES: C[C@@]1(O)COCCN(c2nc(OC[C@@]34CCCN3C[C@H](F)C4)nc3sc4c(-c5c6c(cc7[nH]ncc57)CC5CC65)nccc4c23)C1] | (6S)-4-(2-(((2R,7aS)-2- fluorotetrahydro-1H- pyrrolizin-7a(5H)- yl)methoxy)-8- (4b,5,5a,6-tetrahydro- 1H- cyclopropa[4,5]cyclopen- ta[1,2-f]indazol-4- yl)pyrido[4′,3′:4,5]thieno [2,3-d]pyrimidin-4-yl)- 6-methyl-1,4-oxazepan- 6-ol | | 642 |
| Ex- 91 | 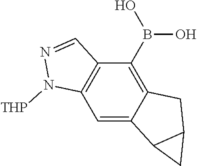 Int-76 | 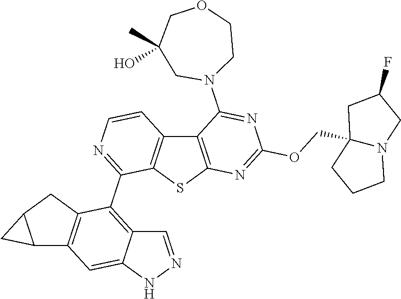 [SMILES: C[C@@]1(O)COCCN(c2nc(OC[C@@]34CCCN3C[C@H](F)C4)nc3sc4c(-c5c6c(cc7[nH]ncc57)C5CC5C6)nccc4c23)C1] | (6S)-4-(2-(((2R,7aS)-2- fluorotetrahydro-1H- pyrrolizin-7a(5H)- yl)methoxy)-8- (5,5a,6,6a-tetrahydro- 1H- cyclopropa[3,4]cyclopen- ta[1,2-f]indazol-4- yl)pyrido[4′,3′:4,5]thieno [2,3-d]pyrimidin-4-yl)- 6-methyl-1,4-oxazepan- 6-ol | | 642 |
| Ex- 92 | 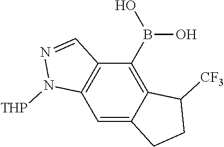 Int-87 | 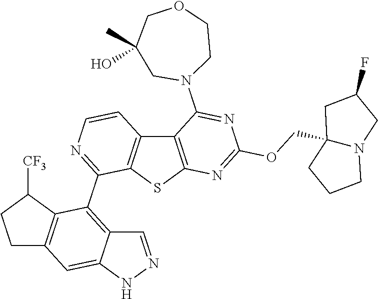 [SMILES: C[C@@]1(O)COCCN(c2nc(OC[C@@]34CCCN3C[C@H](F)C4)nc3sc4c(-c5c6c(cc7[nH]ncc57)CCC6C(F)(F)F)nccc4c23)C1] | (6S)-4-(2-(((2R,7aS)-2- fluorotetrahydro-1H- pyrrolizin-7a(5H)- yl)methoxy)-8-(5- (trifluoromethyl)- 1,5,6,7- tetrahydrocyclopenta[f]in- dazol-4- yl)pyrido[4′,3′:4,5]thieno [2,3-d]pyrimidin-4-y])- 6-methy1-1,4-oxazepan- 6-ol | | 698 |
| Ex- 93 | 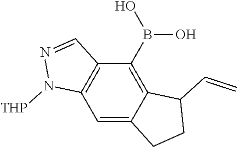 Int-88 | 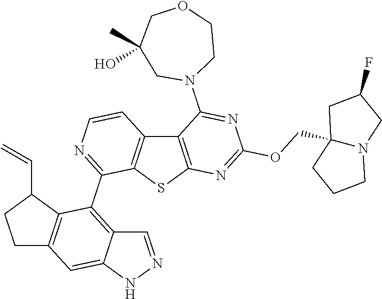 [SMILES: C=CC1CCc2cc3[nH]ncc3c(-c3nccc4c3sc3nc(OC[C@@]56CCCN5C[C@H](F)C6)nc(N5CCOC[C@@](C)(O)C5)c34)c21] | (6S)-4-(2-(((2R,7aS)-2- fluorotetrahydro-1H- pyrrolizin-7a(5H)- yl)methoxy)-8-(5-vinyl- 1,5,6,7- tetrahydrocyclopenta[f]in- dazol-4- yl)pyrido[4′,3′:4,5]thieno [2,3-d]pyrimidin-4-yl)- 6-methyl-1,4-oxazepan- 6-ol | Column P, 40% EtOH in hexane w/ 0.1% Et₂NH, first eluting isomer | 656 |
| Ex- 94 | 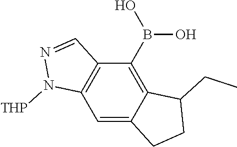 Int-91 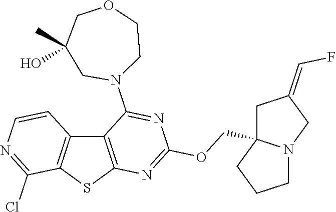 [SMILES: C[C@@]1(O)COCCN(c2nc(OC[C@@]34CCCN3C/C(=C\F)C4)nc3sc4c(Cl)nccc4c23)C1] | 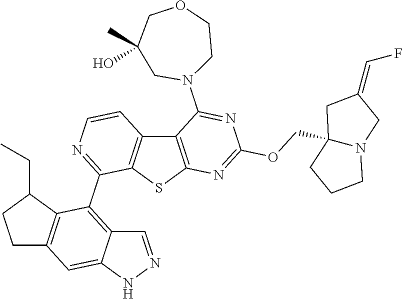 [SMILES: CCC1CCc2cc3[nH]ncc3c(-c3nccc4c3sc3nc(OC[C@@]56CCCN5C/C(=C\F)C6)nc(N5CCOC[C@@](C)(O)C5)c34)c21] | (6S)-4-(8-(5-ethyl- 1,5,6,7- tetrahydrocyclopenta[f]in- dazol-4-y1)-2-(((S,Z)- 2- (fluoromethylene)tetrahy- dro-1H-pyrrolizin- 7a(5H)- yl)methoxy)pyrido[4′,3':4,5]thi- eno[2,3- d]pyrimidin-4-y1)-6- methyl-1,4-oxazepan-6- ol | | 670 |
| | Int-145 | | | | |
| Ex- 95 | 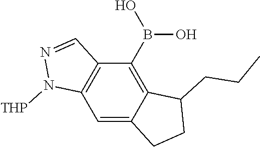 Int-92 | 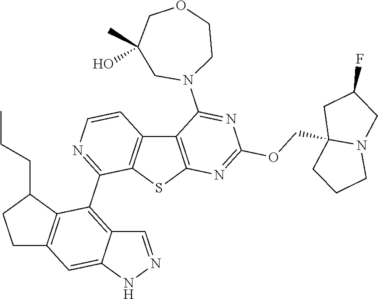 [SMILES: CCCC1CCc2cc3[nH]ncc3c(-c3nccc4c3sc3nc(OC[C@@]56CCCN5C[C@H](F)C6)nc(N5CCOC[C@@](C)(O)C5)c34)c21] | (6S)-4-(2-(((2R,7aS)-2- fluorotetrahydro-1H- pyrrolizin-7a(5H)- yl)methoxy)-8-(5- propyl-1,5,6,7- tetrahydrocyclopenta[f]in- dazol-4- yl)pyrido[4′,3′:4,5]thieno [2,3-d]pyrimidin-4-y])- 6-methyl-1,4-oxazepan- 6-ol | | 672 |
| Ex- 96 | 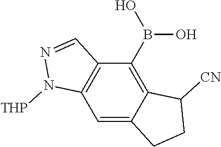 Int-99 | 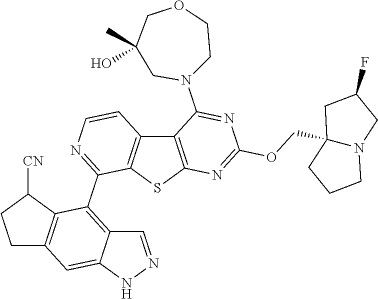 [SMILES: C[C@@]1(O)COCCN(c2nc(OC[C@@]34CCCN3C[C@H](F)C4)nc3sc4c(-c5c6c(cc7[nH]ncc57)CCC6C#N)nccc4c23)C1] | 4-(2-(((2R,7aS)-2- fluorotetrahydro-1H- pyrrolizin-7a(5H)- yl)methoxy)-4-((S)-6- hydroxy-6-methyl-1,4- oxazepan-4- yl)pyrido[4′,3′:4,5]thieno [2,3-d]pyrimidin-8-yl)- 1,5,6,7- tetrahydrocyclopenta[f]in- dazole-5-carbonitrile | | 655 |
| Ex- 97 | 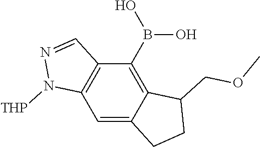 Int-89 | 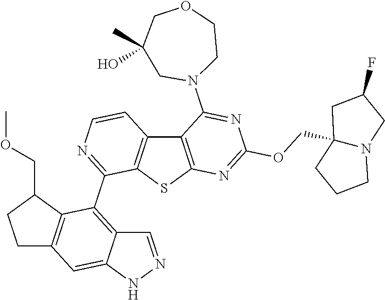 [SMILES: COCC1CCc2cc3[nH]ncc3c(-c3nccc4c3sc3nc(OC[C@@]56CCCN5C[C@H](F)C6)nc(N5CCOC[C@@](C)(O)C5)c34)c21] | (6S)-4-(2-(((2R,7aS)-2- fluorotetrahydro-1H- pyrrolizin-7a(5H)- yl)methoxy)-8-(5- (methoxymethyl)- 1,5,6,7- tetrahydrocyclopenta[f]in- dazol-4- yl)pyrido[4′,3′:4,5]thieno [2,3-d]pyrimidin-4-yl)- 6-methyl-1,4-oxazepan- 6-ol | Column P, 40% EtOH in hexane w/ 0.1% Et₂NH, first eluting isomer | 674 |
| Ex- 98 | 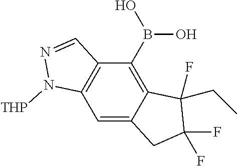 Int-102 | 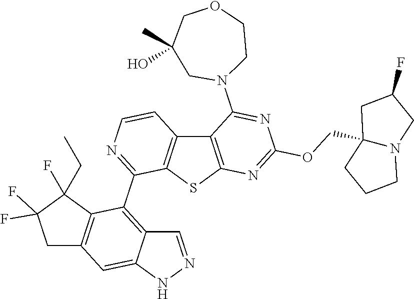 [SMILES: CCC1(F)c2c(cc3[nH]ncc3c2-c2nccc3c2sc2nc(OC[C@@]45CCCN4C[C@H](F)C5)nc(N4CCOC[C@@](C)(O)C4)c23)CC1(F)F] | (6S)-4-(8-(5-ethy1-5,6,6- trifluoro-1,5,6,7- tetrahydrocyclopenta[f]in- dazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 712 |
| Ex- 99 | 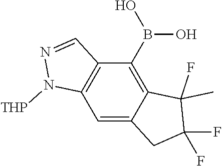 Int-103 | 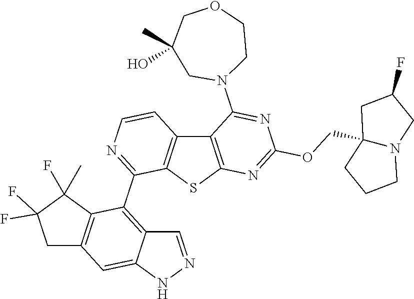 [SMILES: CC1(F)c2c(cc3[nH]ncc3c2-c2nccc3c2sc2nc(OC[C@@]45CCCN4C[C@H](F)C5)nc(N4CCOC[C@@](C)(O)C4)c23)CC1(F)F] | (6S)-4-(2-(((2R,7aS)-2- fluorotetrahydro-1H- pyrrolizin-7a(5H)- yl)methoxy)-8-(5,6,6- trifluoro-5-methyl- 1,5,6,7- tetrahydrocyclopenta[f]in- dazol-4- yl)pyrido[4′,3′:4,5]thieno [2,3-d]pyrimidin-4-yl)- 6-methyl-1,4-oxazepan- 6-o1 | Prep reverse- phase HPLC (MeCN/ H₂O w/ formic acid modifier) first and third eluting peak | 698 |
| Ex- 100 | 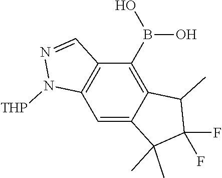 Int-101 | 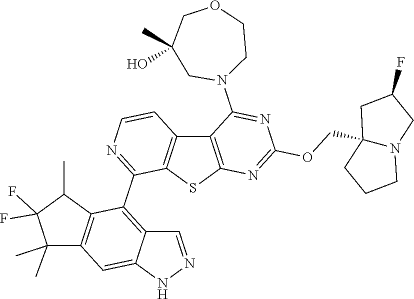 [SMILES: CC1c2c(cc3[nH]ncc3c2-c2nccc3c2sc2nc(OC[C@@]45CCCN4C[C@H](F)C5)nc(N4CCOC[C@@](C)(O)C4)c23)C(C)(C)C1(F)F] | (6S)-4-(8-(6,6-difluoro- 5,7,7-trimethyl-1,5,6,7- tetrahydrocyclopenta[f]in- dazol-4-yl)-2- (((2R,7aS)-2- fluorotetrahydro-1H- pyrrolizin-7a(5H)- yl)methoxy)pyrido[4′,3′: 4,5]thieno[2,3- d]pyrimidin-4-yl)-6- methyl-1,4-oxazepan-6- ol | | 708 |
| Ex- 101 | 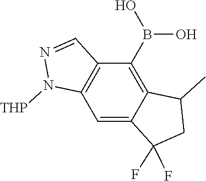 Int-108 | 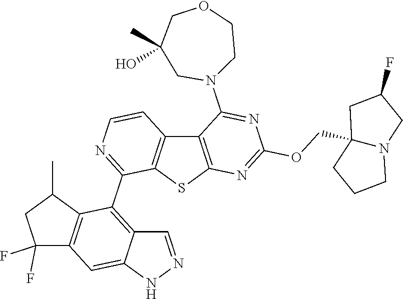 [SMILES: CC1CC(F)(F)c2cc3[nH]ncc3c(-c3nccc4c3sc3nc(OC[C@@]56CCCN5C[C@H](F)C6)nc(N5CCOC[C@@](C)(O)C5)c34)c21] | (6S)-4-(8-(7,7-difluoro- 5-methy1-1,5,6,7- tetrahydrocyclopenta[f]in- dazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 680 |
| Ex- 102 | 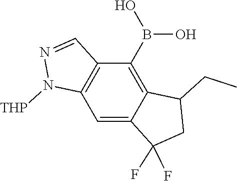 Int-109 | 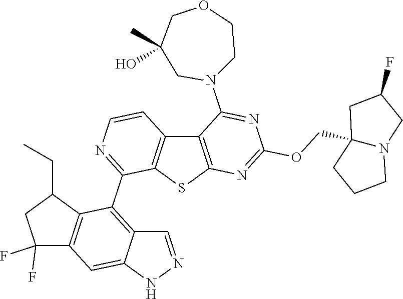 [SMILES: CCC1CC(F)(F)c2cc3[nH]ncc3c(-c3nccc4c3sc3nc(OC[C@@]56CCCN5C[C@H](F)C6)nc(N5CCOC[C@@](C)(O)C5)c34)c21] | (6S)-4-(8-(5-ethyl-7,7- difluoro-1,5,6,7- tetrahydrocyclopenta[f]in- dazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 694 |
| Ex- 103 | 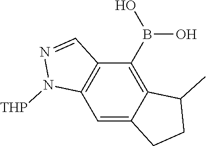 Int-83 | 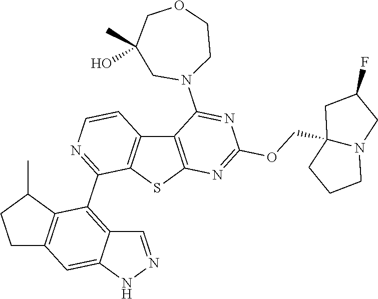 [SMILES: CC1CCc2cc3[nH]ncc3c(-c3nccc4c3sc3nc(OC[C@@]56CCCN5C[C@H](F)C6)nc(N5CCOC[C@@](C)(O)C5)c34)c21] | (6S)-4-(2-(((2R,7aS)-2- fluorotetrahydro-1H- pyrrolizin-7a(5H)- yl)methoxy)-8-(5- methyl-1,5,6,7- tetrahydrocyclopenta[f]in- dazol-4- yl)pyrido[4′,3′:4,5]thieno [2,3-d]pyrimidin-4-y])- 6-methy1-1,4-oxazepan- 6-ol | Column P, 40% EtOH in hexane w/ 0.1% Et₂NH, first eluting isomer | 644 |
| Ex- 104 | 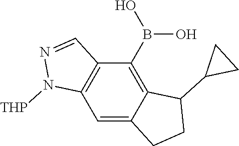 Int-94 | 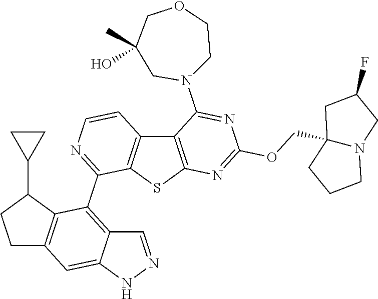 [SMILES: C[C@@]1(O)COCCN(c2nc(OC[C@@]34CCCN3C[C@H](F)C4)nc3sc4c(-c5c6c(cc7[nH]ncc57)CCC6C5CC5)nccc4c23)C1] | (6S)-4-(8-(5- cyclopropyl-1,5,6,7- tetrahydrocyclopenta[f]in- dazol-4-yl)-2- (((2R,7aS)-2- fluorotetrahydro-1H- pyrrolizin-7a(5H)- yl)methoxy)pyrido[4′,3′:4,5] thieno[2,3- d]pyrimidin-4-yl)-6- methyl-1,4-oxazepan-6- ol | | 670 |
| Ex- 105 | 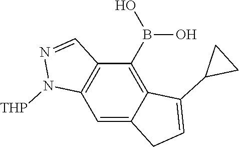 Int-93 | 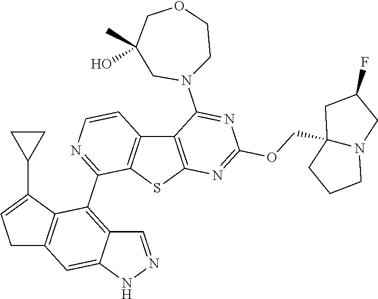 [SMILES: C[C@@]1(O)COCCN(c2nc(OC[C@@]34CCCN3C[C@H](F)C4)nc3sc4c(-c5c6c(cc7[nH]ncc57)CC=C6C5CC5)nccc4c23)C1] | (6S)-4-(8-(5- cyclopropyl-1,7- dihydrocyclopenta[f]inda- zol-4-yl)-2-(((2R,7aS)- 2-fluorotetrahydro-1H- pyrrolizin-7a(5H)- yl)methoxy)pyrido[4′,3′:4,5] thieno[2,3- d]pyrimidin-4-yl)-6- methyl-1,4-oxazepan-6- ol | | 668 |
| Ex 106 | 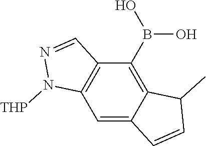 Int-111 | 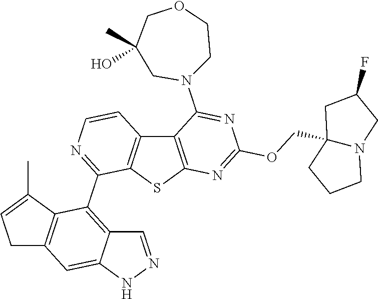 [SMILES: CC1=CCc2cc3[nH]ncc3c(-c3nccc4c3sc3nc(OC[C@@]56CCCN5C[C@H](F)C6)nc(N5CCOC[C@@](C)(O)C5)c34)c21] | (6S)-4-(2-(((2R,7aS)-2- fluorotetrahydro-1H- pyrrolizin-7a(5H)- yl)methoxy)-8-(5- methyl-1,7- dihydrocyclopenta[f]inda- zol-4- yl)pyrido[4′,3′:4,5]thieno [2,3-d]pyrimidin-4-yl)- 6-methyl-1,4-oxazepan- 6-ol | | 642 |
| Ex- 107 | 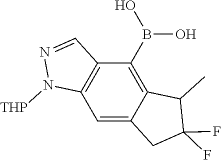 Int-100 | 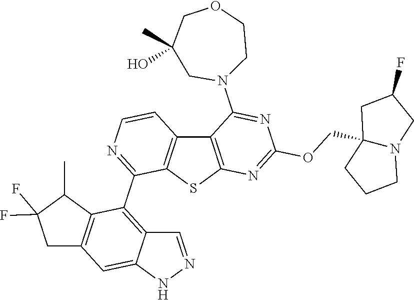 [SMILES: CC1c2c(cc3[nH]ncc3c2-c2nccc3c2sc2nc(OC[C@@]45CCCN4C[C@H](F)C5)nc(N4CCOC[C@@](C)(O)C4)c23)CC1(F)F] | (6S)-4-(8-(6,6-difluoro- 5-methyl-1,5,6,7- tetrahydrocyclopenta[f]in- dazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | Column P, 40% EtOH in hexane w/ 0.1% Et₂NH, first eluting isomer | 680 |
| Ex- 108 | 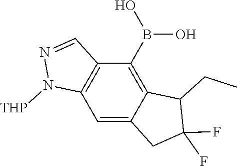 Int-104 | 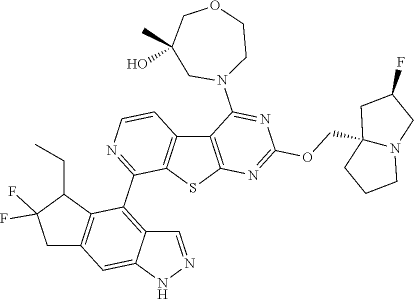 [SMILES: CCC1c2c(cc3[nH]ncc3c2-c2nccc3c2sc2nc(OC[C@@]45CCCN4C[C@H](F)C5)nc(N4CCOC[C@@](C)(O)C4)c23)CC1(F)F] | (6S)-4-(8-(5-ethyl-6,6- difluoro-1,5,6,7- tetrahydrocyclopenta[f]in- dazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | Column P, 40% EtOH in hexane w/ 0.1% Et₂NH, first eluting isomer | 694 |
| Ex- 109 | 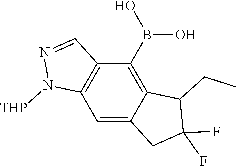 Int-104 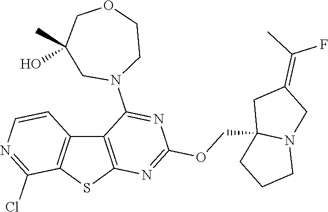 [SMILES: C/C(F)=C1/CN2CCC[C@@]2(COc2nc(N3CCOC[C@@](C)(O)C3)c3c(n2)sc2c(Cl)nccc23)C1] | 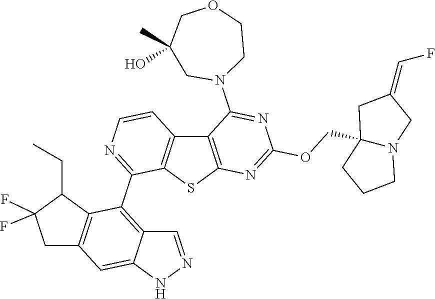 [SMILES: CCC1c2c(cc3[nH]ncc3c2-c2nccc3c2sc2nc(OC[C@@]45CCCN4C/C(=C\F)C5)nc(N4CCOC[C@@](C)(O)C4)c23)CC1(F)F] | (6S)-4-(8-(5-ethyl-6,6- difluoro-1,5,6,7- tetrahydrocyclopenta[f]in- dazol-4-yl)-2-(((S,Z)- 2- (fluoromethylene)tetrahy- dro-1H-pyrrolizin- 7a(5H)- yl)methoxy)pyrido[4′,3′:4,5]thi- eno[2,3- d]pyrimidin-4-yl)-6- methy1-1,4-oxazepan-6- ol | Column P, 40% EtOH in hexane w/ 0.1% Et₂NH, first eluting isomer | 706 |
| | Int-105 | | | | |
| Ex- 110 | 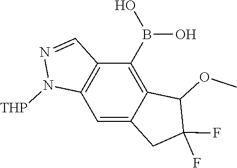 Int-105 | 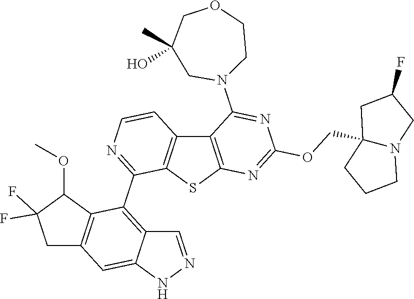 [SMILES: COC1c2c(cc3[nH]ncc3c2-c2nccc3c2sc2nc(OC[C@@]45CCCN4C[C@H](F)C5)nc(N4CCOC[C@@](C)(O)C4)c23)CC1(F)F] | (6S)-4-(8-(6,6-difluoro- 5-methoxy-1,5,6,7- tetrahydrocyclopenta[f]in- dazol-4-yl)-2- (((2R,7aS)-2- fluorotetrahydro-1H- pyrrolizin-7a(5H)- yl)methoxy)pyrido[4′,3′:4,5]thi- no[2,3- d]pyrimidin-4-yl)-6- methyl-1,4-oxazepan-6- ol | | 696 |
| Ex- 111 | 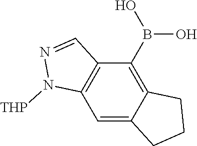 Int-95 | 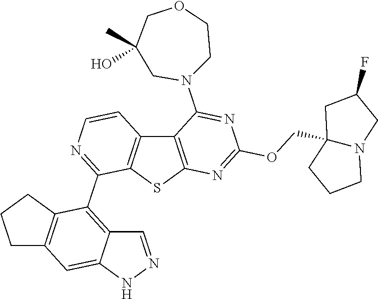 [SMILES: C[C@@]1(O)COCCN(c2nc(OC[C@@]34CCCN3C[C@H](F)C4)nc3sc4c(-c5c6c(cc7[nH]ncc57)CCC6)nccc4c23)C1] | (6S)-4-(2-(((2R,7aS)-2- fluorotetrahydro-1H- pyrrolizin-7a(5H)- yl)methoxy)-8-(1,5,6,7- tetrahydrocyclopenta[f]in- dazol-4- yl)pyrido[4′,3′:4,5]thieno [2,3-d]pyrimidin-4-yl)- 6-methyl-1,4-oxazepan- 6-ol | | 630 |
| Ex- 112 | 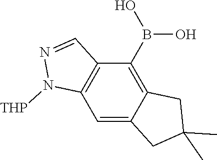 Int-98 | 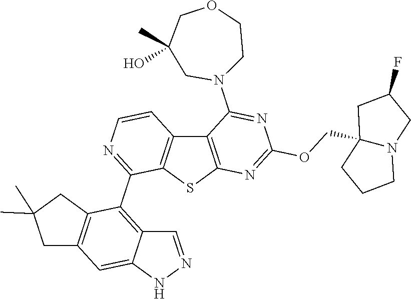 [SMILES: CC1(C)Cc2cc3[nH]ncc3c(-c3nccc4c3sc3nc(OC[C@@]56CCCN5C[C@H](F)C6)nc(N5CCOC[C@@](C)(O)C5)c34)c2C1] | (6S)-4-(8-(6,6-dimethyl- 1,5,6,7- tetrahydrocyclopenta[f]in- dazol-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 658 |
| Ex- 113 | 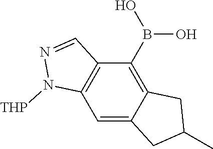 Int-97 | 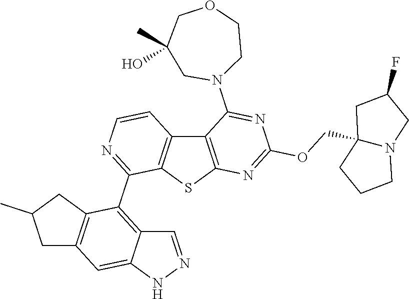 [SMILES: CC1Cc2cc3[nH]ncc3c(-c3nccc4c3sc3nc(OC[C@@]56CCCN5C[C@H](F)C6)nc(N5CCOC[C@@](C)(O)C5)c34)c2C1] | (6S)-4-(2-(((2R,7aS)-2- fluorotetrahydro-1H- pyrrolizin-7a(5H)- yl)methoxy)-8-(6- methyl-1,5,6,7- tetrahydrocyclopenta[f]in- dazol-4- yl)pyrido[4′,3′:4,5]thieno [2,3-d]pyrimidin-4-yl)- 6-methy1-1,4-oxazepan- 6-ol | | 644 |
| Ex- 114 | 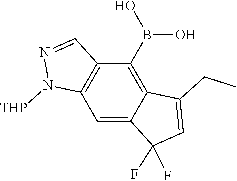 Int-107 | 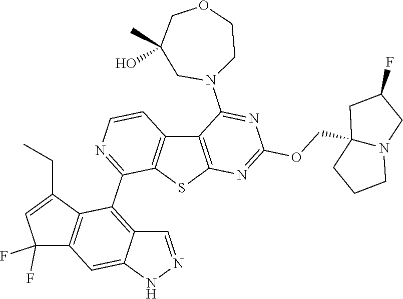 [SMILES: CCC1=CC(F)(F)c2cc3[nH]ncc3c(-c3nccc4c3sc3nc(OC[C@@]56CCCN5C[C@H](F)C6)nc(N5CCOC[C@@](C)(O)C5)c34)c21] | (6S)-4-(8-(5-ethyl-7,7- difluoro-1,7- dihydrocyclopenta[f]inda- zol-4-yl)-2-(((2R,7aS)- 2-fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 692 |
| Ex- 115 | 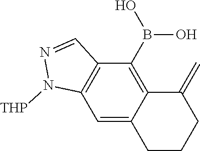 Int-81 | 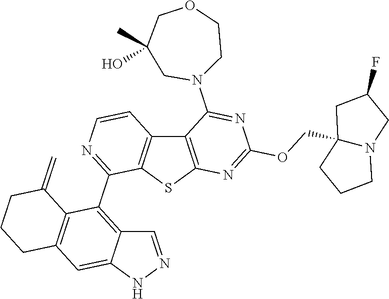 [SMILES: C=C1CCCc2cc3[nH]ncc3c(-c3nccc4c3sc3nc(OC[C@@]56CCCN5C[C@H](F)C6)nc(N5CCOC[C@@](C)(O)C5)c34)c21] | (6S)-4-(2-(((2R,7aS)-2- fluorotetrahydro-1H- pyrrolizin-7a(5H)- yl)methoxy)-8-(5- methylene-5,6,7,8- tetrahydro-1H- benzo[f]indazol-4- yl)pyrido[4′,3′:4,5]thieno [2,3-d]pyrimidin-4-yl)- 6-methyl-1,4-oxazepan- 6-ol | | 656 |
| Ex- 116 | 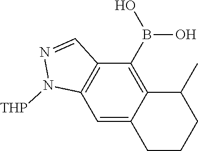 Int-80 | 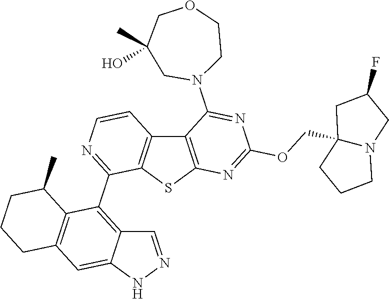 [SMILES: C[C@@H]1CCCc2cc3[nH]ncc3c(-c3nccc4c3sc3nc(OC[C@@]56CCCN5C[C@H](F)C6)nc(N5CCOC[C@@](C)(O)C5)c34)c21] | (6S)-4-(2-(((2R,7aS)-2- fluorotetrahydro-1H- pyrrolizin-7a(5H)- yl)methoxy 8-((R)-5- methyl-5,6,7,8- tetrahydro-1H- benzo[f]indazol-4- yl)pyrido[4′,3′:4,5]thieno [2,3-d]pyrimidin-4-yl)- 6-methyl-1,4-oxazepan- 6-ol | Column D, 45% MeOH w/ 0.1% NH₄OH, 4th eluting isomer | 658 |
| Ex- 117 | 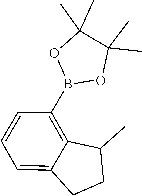 [SMILES: CC1CCc2cccc(B3OC(C)(C)C(C)(C)O3)c21] Int-113 | 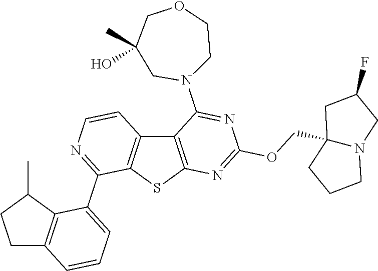 [SMILES: CC1CCc2cccc(-c3nccc4c3sc3nc(OC[C@@]56CCCN5C[C@H](F)C6)nc(N5CCOC[C@@](C)(O)C5)c34)c21] | (6S)-4-(2-(((2R,7aS)-2- fluorotetrahydro-1H- pyrrolizin-7a(5H)- yl)methoxy)-8-(3- methyl-2,3-dihydro-1H- inden-4- yl)pyrido[4′,3′:4,5]thieno [2,3-d]pyrimidin-4-yl)- 6-methyl-1,4-oxazepan- 6-ol | Column H, 45% MeOH w/ 0.1% NH₄OH, First eluting isomer | 604 |
| Ex- 118 | 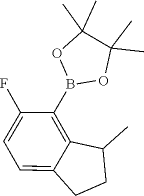 [SMILES: CC1CCc2ccc(F)c(B3OC(C)(C)C(C)(C)O3)c21] Int-114 | 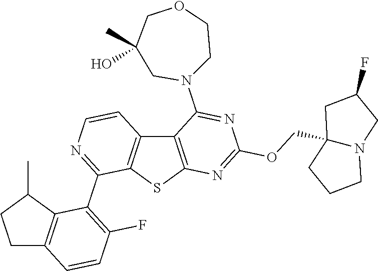 [SMILES: CC1CCc2ccc(F)c(-c3nccc4c3sc3nc(OC[C@@]56CCCN5C[C@H](F)C6)nc(N5CCOC[C@@](C)(O)C5)c34)c21] | (6S)-4-(8-(5-fluoro-3- methyl-2,3-dihydro-1H- inden-4-yl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | Column C, 45% MeOH w/ 0.1% NH₄OH First eluting isomer | 622 |
| Ex- 119 | 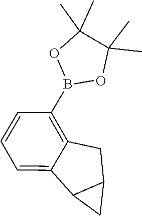 [SMILES: CC1(C)OB(c2cccc3c2CC2CC32)OC1(C)C] Int-115 | 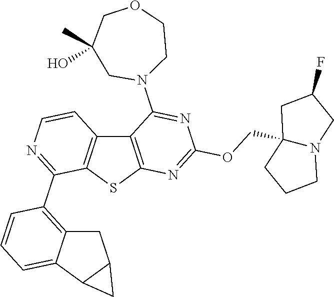 [SMILES: C[C@@]1(O)COCCN(c2nc(OC[C@@]34CCCN3C[C@H](F)C4)nc3sc4c(-c5cccc6c5CC5CC65)nccc4c23)C1] | (6S)-4-(2-(((2R,7aS)-2- fluorotetrahydro-1H- pyrrolizin-7a(5H)- yl)methoxy)-8- (1,1a,6,6a- tetrahydrocyclopropa[a] inden-5- yl)pyrido[4′,3′:4,5]thieno [2,3-d]pyrimidin-4-yl)- 6-methyl-1,4-oxazepan- 6-ol | Column Q, 35% MeOH w/ 0.1% NH₄OH, First eluting isomer | 602 |
| Ex 120 | 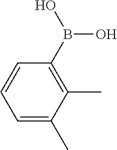 | 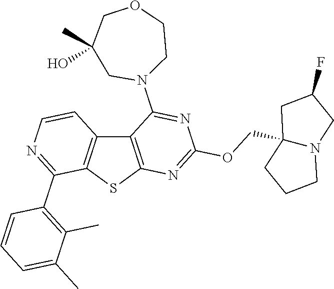 [SMILES: Cc1cccc(-c2nccc3c2sc2nc(OC[C@@]45CCCN4C[C@H](F)C5)nc(N4CCOC[C@@](C)(O)C4)c23)c1C] | (S)-4-(8-(2,3- dimethylpheny1)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 578 |
| Ex- 121 | 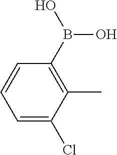 | 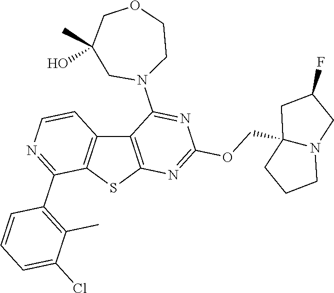 [SMILES: Cc1c(Cl)cccc1-c1nccc2c1sc1nc(OC[C@@]34CCCN3C[C@H](F)C4)nc(N3CCOC[C@@](C)(O)C3)c12] | (S)-4-(8-(3-chloro-2- methylphenyl)-2- (((2R,7aS)-2- fluorotetrahydro-1H- pyrrolizin-7a(5H)- yl)methoxy)pyrido[4′,3′:4,5]thi- eno[2,3- d]pyrimidin-4-yl)-6- methyl-1,4-oxazepan-6- ol | | 598 |
| Ex- 122 | 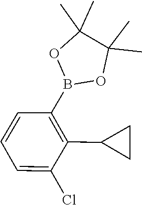 [SMILES: CC1(C)OB(c2cccc(Cl)c2C2CC2)OC1(C)C] | 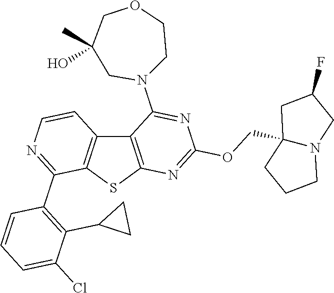 [SMILES: C[C@@]1(O)COCCN(c2nc(OC[C@@]34CCCN3C[C@H](F)C4)nc3sc4c(-c5cccc(Cl)c5C5CC5)nccc4c23)C1] | (S)-4-(8-(3-chloro-2- cyclopropylphenyl)-2- (((2R,7aS)-2- fluorotetrahydro-1H- pyrrolizin-7a(5H)- yl)methoxy)pyri- do[4′,3′:4,5]thieno[2,3- d]pyrimidin-4-yl)-6- methyl-1,4-oxazepan-6- ol | | 624 |
| Ex- 123 | 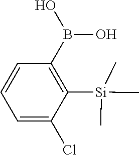 Int-116 | 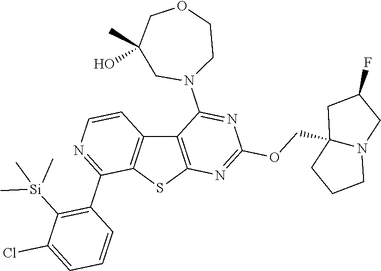 [SMILES: C[C@@]1(O)COCCN(c2nc(OC[C@@]34CCCN3C[C@H](F)C4)nc3sc4c(-c5cccc(Cl)c5[Si](C)(C)C)nccc4c23)C1] | (S)-4-(8-(3-chloro-2- (trimethylsilyl)phenyl)- 2-(((2R,7aS)-2- fluorotetrahydro-1H- pyrrolizin-7a(5H)- yl)methoxy)pyri- do[4′,3′:4,5]thieno[2,3- d]pyrimidin-4-yl)-6- methyl-1,4-oxazepan-6- ol | | 656 |
| Ex- 124 | 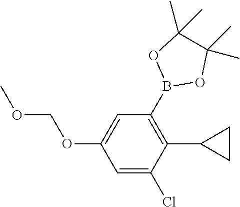 | 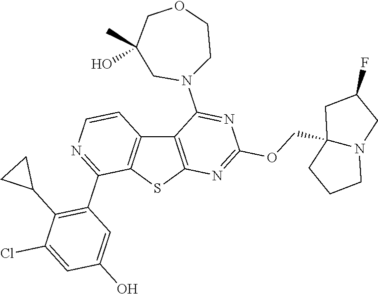 [SMILES: C[C@@]1(O)COCCN(c2nc(OC[C@@]34CCCN3C[C@H](F)C4)nc3sc4c(-c5cc(O)cc(Cl)c5C5CC5)nccc4c23)C1] | (S)-4-(8-(3-chloro-2- cyclopropyl-5- hydroxyphenyl)-2- (((2R,7aS)-2- fluorotetrahydro-1H- pyrrolizin-7a(5H)- yl)methoxy)pyri- do[4′,3′:4,5]thieno[2,3- d]pyrimidin-4-yl)-6- methyl-1,4-oxazepan-6- ol | | 640 |

### Example 125: (6S)-4-(2-(((2R,7aS)-2-fluorotetrahydro-1H-pyrrolizin-7a(5H)-yl)methoxy)-8-(5-methyl-1,5,6,7-tetrahydropyrrolo[2,3-f]indazol-4-yl)pyrido[4′,3′:4,5]thieno[2,3-d]pyrimidin-4-yl)-6-methyl-1,4-oxazepan-6-ol (Ex-125)

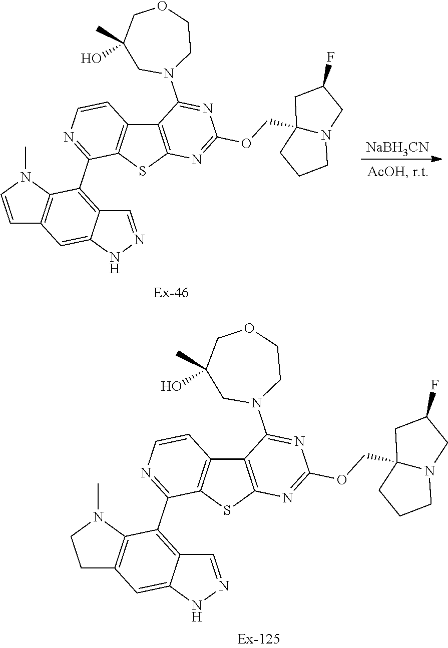 [SMILES: Cn1ccc2cc3[nH]ncc3c(-c3nccc4c3sc3nc(OC[C@@]56CCCN5C[C@H](F)C6)nc(N5CCOC[C@@](C)(O)C5)c34)c21 ; "NaBH3CN" ; CN1CCc2cc3[nH]ncc3c(-c3nccc4c3sc3nc(OC[C@@]56CCCN5C[C@H](F)C6)nc(N5CCOC[C@@](C)(O)C5)c34)c21]

To a solution of (6S)-4-(2-(((2R,7aS)-2-fluorotetrahydro-1H-pyrrolizin-7a(5H)-yl)methoxy)-8-(5-methyl-1,5-dihydropyrrolo[2,3-f]indazol-4-yl)pyrido[4′,3′:4,5]thieno[2,3-d]pyrimidin-4-yl)-6-methyl-1,4-oxazepan-6-ol (Ex-46) (45 mg, 0.070 mmol) in AcOH (0.5 mL) was added NaBH₃CN (8.80 mg, 0.140 mmol) at 25° C. The mixture was stirred at 25° C. for 1 h. The solvent was removed under reduced pressure and the crude was purified by preparative reverse-phase HPLC (MeCN/H₂O w/formic acid modifier) to give (6S)-4-(2-(((2R,7aS)-2-fluorotetrahydro-1H-pyrrolizin-7a(5H)-yl)methoxy)-8-(5-methyl-1,5,6,7-tetrahydropyrrolo[2,3-f]indazol-4-yl)pyrido[4′,3′:4,5]thieno[2,3-d]pyrimidin-4-yl)-6-methyl-1,4-oxazepan-6-ol (Ex-125). MS (ESI) [M+H]⁺: m/z 645. ¹H NMR (400 MHz, CD₃OD) δ 8.62 (dd, J=2.26, 5.60 Hz, 1H), 7.75 (dd, J=2.62, 5.60 Hz, 1H), 7.24-7.32 (m, 2H), 5.11-5.29 (m, 1H), 4.18-4.24 (m, 1H), 4.01-4.12 (m, 2H), 3.97 (d, J=14.90 Hz, 1H), 3.71-3.85 (m, 3H), 3.53-3.67 (m, 2H), 3.36-3.49 (m, 2H), 3.25-3.32 (m, 1H), 3.15 (br s, 2H), 3.03-3.10 (m, 3H), 2.86-2.96 (m, 1H), 2.10-2.27 (m, 2H), 2.07 (d, J=13.47 Hz, 3H), 1.97-2.03 (m, 1H), 1.85-1.93 (m, 2H), 1.75-1.83 (m, 1H), 1.05-1.12 (m, 1H), 1.05-1.12 (m, 2H).

### Example 126: (6S)-4-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidin-4-yl)-6-methyl-1,4-oxazepan-6-ol (Ex-126)

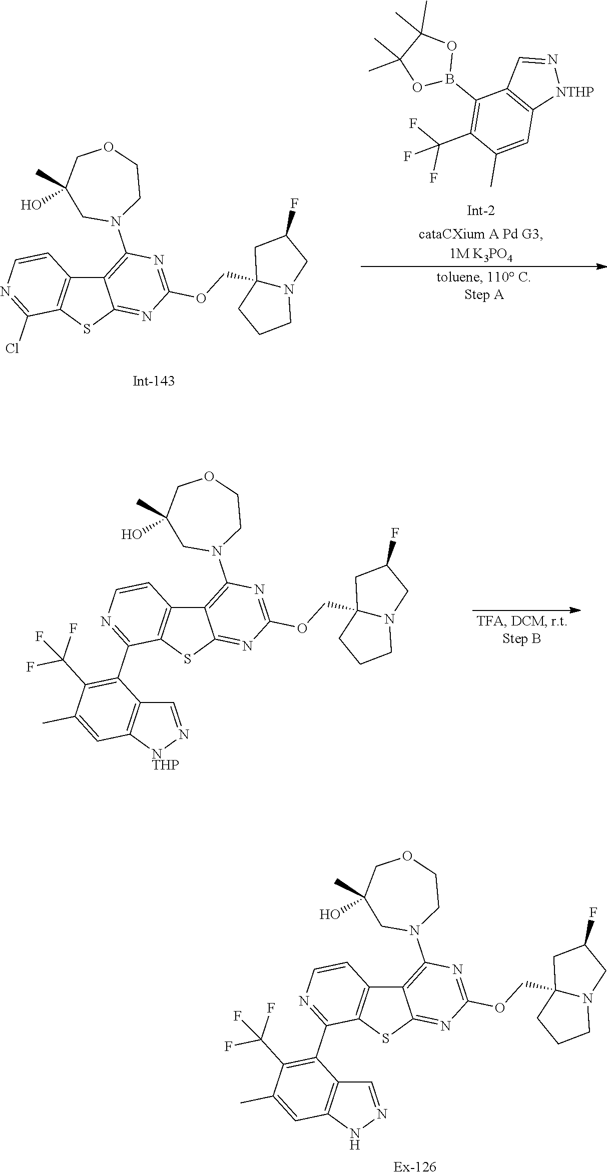

### Step A: (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidin-4-yl)-6-methyl-1,4-oxazepan-6-ol

A mixture of(S)-4-(8-chloro-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Int-143) (180 mg, 0.354 mmol), 6-methyl-1-(tetrahydro-2H-pyran-2-yl)-4-(4,4,5,5-tetramethyl-1,3,2-dioxaborolan-2-yl)-5-(trifluoromethyl)-1H-indazole (Int-2) (218 mg, 0.513 mmol), cataCXium A Pd G3 (129 mg, 0.177 mmol) and IM aq. K₃PO₄ (1.06 mL, 1.06 mmol) in toluene (4 ml) was degassed with nitrogen three times. Then the mixture was heated to 110° C. and stirred under nitrogen atmosphere for 5 h. After cooling, the mixture was partitioned between EtOAc and water. The organic phase was washed with brine, dried over sodium sulfate, filtered, and concentrated. The crude residue was purified via silica gel chromatography (0% to 30% MeOH in EtOAc) to give (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidin-4-yl)-6-methyl-1,4-oxazepan-6-ol. MS (ESI): m/z (M+H)⁺ 756.

### Step B: (6S)-4-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidin-4-yl)-6-methyl-1,4-oxazepan-6-ol (Ex-126)

To a stirred solution of (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidin-4-yl)-6-methyl-1,4-oxazepan-6-ol (150 mg, 0.199 mmol) in DCM (1.2 ml) was added TFA (500 μl) and the reaction was allowed to stir at r.t. for 1 h. After 1 h, the reaction was concentrated in vacuo. The residue was partitioned between sat. aq. sodium bicarbonate and chloroform. The organic phase was washed with brine, dried over sodium sulfate, filtered, and concentrated. The crude residue was purified via silica gel chromatography (0% to 25% MeOH in chloroform) to give (6S)-4-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidin-4-yl)-6-methyl-1,4-oxazepan-6-ol (Ex-126). MS (ESI): (M+H)⁺ 672. ¹H NMR (400 MHz, CDCl₃) δ=8.83-8.77 (m, 1H), 7.66-7.62 (m, 1H), 7.58 (s, 1H), 7.47 (m, 1H), 6.63-6.47 (m, 1H), 5.41-5.17 (m, 1H), 4.29-4.07 (m, 4H), 4.00 (td, J=3.6, 12.5 Hz, 1H), 3.83 (d, J=12.5 Hz, 1H), 3.81-3.71 (m, 1H), 3.67 (d, J=14.9 Hz, 1H), 3.50-3.31 (m, 2H), 3.31-3.21 (m, 2H), 3.20-3.09 (m, 1H), 3.05-2.90 (m, 1H), 2.79-2.72 (m, 3H), 2.29-2.03 (m, 3H), 2.00-1.81 (m, 3H), 1.41 (s, 3H).

The examples in the table below were synthesized using a similar procedure as described in the synthesis of Ex-126 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | | [M + H]⁺ |
|---|---|---|---|---|
| Ex. | Starting Material | Structure | Compound Name | Found |
| Ex- 127 | 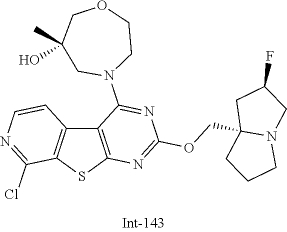 [SMILES: C[C@@]1(O)COCCN(c2nc(OC[C@@]34CCCN3C[C@H](F)C4)nc3sc4c(Cl)nccc4c23)C1] | 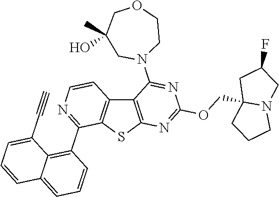 [SMILES: C#Cc1cccc2cccc(-c3nccc4c3sc3nc(OC[C@@]56CCCN5C[C@H](F)C6)nc(N5CCOC[C@@](C)(O)C5)c34)c12] | (S)-4-(8-(8-ethynyl- naphthalen-1-yl)-2- (((2R,7aS)-2-fluoro- tetrahydro-1H-pyr- rolizin-7a(5H)-yl)- methoxy)pyrido[4′, 3′:4,5]thieno[2,3- d]pyrimidin-4-yl)- 6-methyl-1,4-oxaze- pan-6-ol | 624 |
| Ex- 128 | 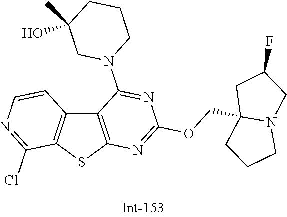 [SMILES: C[C@@]1(O)CCCN(c2nc(OC[C@@]34CCCN3C[C@H](F)C4)nc3sc4c(Cl)nccc4c23)C1] | 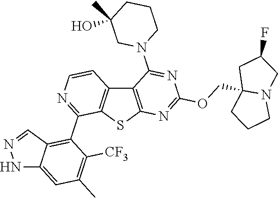 [SMILES: Cc1cc2[nH]ncc2c(-c2nccc3c2sc2nc(OC[C@@]45CCCN4C[C@H](F)C5)nc(N4CCC[C@@](C)(O)C4)c23)c1C(F)(F)F] | (3R)-1-(2-(((2R, 7aS)-2-fluorotetra- hydro-1H-pyrrol- izin-7a(5H)-yl)meth- oxy)-8-(6-methyl-5- (trifluoromethyl)- 1H-indazol-4-yl)- pyrido[4′,3′:4,5] thieno[2,3-d]pyr- imidin-4-yl)-3- methylpiperidin- 3-ol | 656 |
| Ex- 129 | 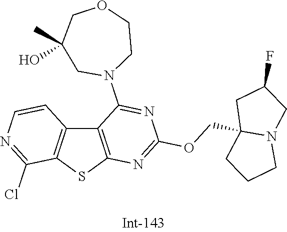 [SMILES: C[C@@]1(O)COCCN(c2nc(OC[C@@]34CCCN3C[C@H](F)C4)nc3sc4c(Cl)nccc4c23)C1] | 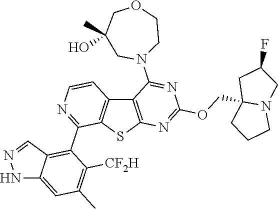 [SMILES: Cc1cc2[nH]ncc2c(-c2nccc3c2sc2nc(OC[C@@]45CCCN4C[C@H](F)C5)nc(N4CCOC[C@@](C)(O)C4)c23)c1C(F)F] | (6S)-4-(8-(5-(di- fluoromethyl)-6- methyl-1H-indazol- 4-yl)-2-(((2R,7aS)- 2-fluorotetrahydro- 1H-pyrrolizin- 7a(5H)-yl)methoxy)- pyrido[4′,3′:4,5] thieno[2,3-d]pyr- imidin-4-yl)-6-meth- yl-1,4-oxazepan- 6-ol | 654 |
| Ex- 130 | 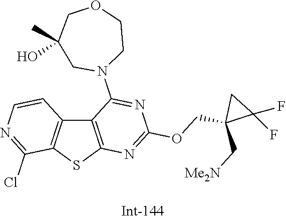 [SMILES: CN(C)C[C@@]1(COc2nc(N3CCOC[C@@](C)(O)C3)c3c(n2)sc2c(Cl)nccc23)CC1(F)F] | 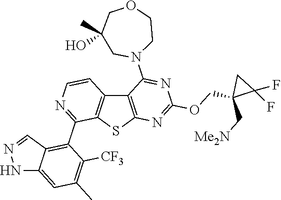 [SMILES: Cc1cc2[nH]ncc2c(-c2nccc3c2sc2nc(OC[C@]4(CN(C)C)CC4(F)F)nc(N4CCOC[C@@](C)(O)C4)c23)c1C(F)(F)F] | (6S)-4-(2-(((R)-1- ((dimethylamino)- methyl)-2,2-di- fluorocyclopropyl)- methoxy)-8-(6- methyl-5-(trifluoro- methyl)-1H-indazol- 4-yl)pyrido[4′,3′: 4,5]thieno[2,3-d] pyrimidin-4-yl)-6- methyl-1,4-oxaze- pan-6-ol | 678 |
| Ex- 131 | 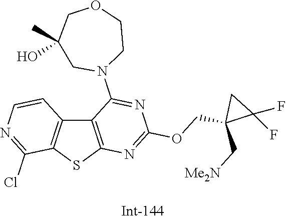 [SMILES: CN(C)C[C@@]1(COc2nc(N3CCOC[C@@](C)(O)C3)c3c(n2)sc2c(Cl)nccc23)CC1(F)F] | 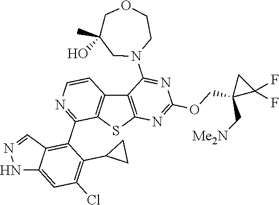 [SMILES: CN(C)C[C@@]1(COc2nc(N3CCOC[C@@](C)(O)C3)c3c(n2)sc2c(-c4c(C5CC5)c(Cl)cc5[nH]ncc45)nccc23)CC1(F)F] | (6S)-4-(8-(6-chloro- 5-cyclopropyl-1H- indazol-4-yl)-2- (((R)-1-((dimeth- ylamino )methyl)- 2,2-difluorocyclo- propyl)methoxy)- pyrido[4′,3′:4,5] thieno[2,3-d]pyr- imidin-4-yl)-6- methyl-1,4-oxaze- pan-6-ol | 670 |
| Ex- 132 | 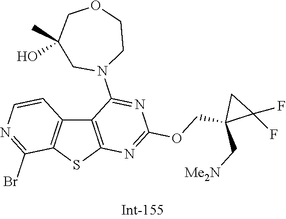 [SMILES: CN(C)C[C@@]1(COc2nc(N3CCOC[C@@](C)(O)C3)c3c(n2)sc2c(Br)nccc23)CC1(F)F] | 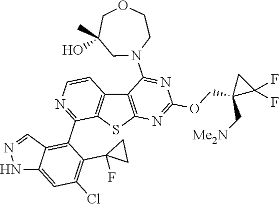 [SMILES: CN(C)C[C@@]1(COc2nc(N3CCOC[C@@](C)(O)C3)c3c(n2)sc2c(-c4c(C5(F)CC5)c(Cl)cc5[nH]ncc45)nccc23)CC1(F)F] | (6S)-4-(8-(6-chloro- 5-(1-fluorocyclo- propyl)-1H-indazol- 4-yl)-2-(((R)-1- ((dimethylamino)- methyl)-2,2-di- fluorocyclopropyl)- methoxy)pyrido [4′,3′:4,5]thieno[2,3- d]pyrimidin-4-yl)-6- methyl-1,4-oxaze- pan-6-ol | 688 |
| Ex- 133 | 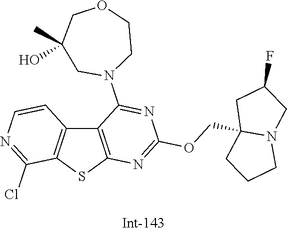 [SMILES: C[C@@]1(O)COCCN(c2nc(OC[C@@]34CCCN3C[C@H](F)C4)nc3sc4c(Cl)nccc4c23)C1] | 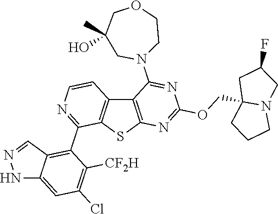 [SMILES: C[C@@]1(O)COCCN(c2nc(OC[C@@]34CCCN3C[C@H](F)C4)nc3sc4c(-c5c(C(F)F)c(Cl)cc6[nH]ncc56)nccc4c23)C1] | (6S)-4-(8-(6-chloro- 5-(difluoromethyl)- 1H-indazol-4-yl)-2- (((2R,7aS)-2-fluoro- tetrahydro-1H-pyr- rolizin-7a(5H)-yl)- methoxy)pyrido [4′,3′:4,5]thieno [2,3-d]pyrimidin- 4-yl)-6-methyl-1,4- oxazepan-6-ol | 674 |
| Ex- 134 | Byproduct isolated from Ex-133 | 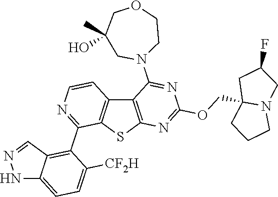 [SMILES: C[C@@]1(O)COCCN(c2nc(OC[C@@]34CCCN3C[C@H](F)C4)nc3sc4c(-c5c(C(F)F)ccc6[nH]ncc56)nccc4c23)C1] | (6S)-4-(8-(5-di- fluoromethyl)-1H- indazol-4-yl)-2- (((2R,7aS)-2-fluoro- tetrahydro-1H-pyr- rolizin-7a(5H)-yl)- methoxy)pyrido [4′,3′:4,5]thieno[2,3- d]pyrimidin-4-yl)-6- methyl-1,4-oxaze- pan-6-ol | 640 |
| Ex- 135 | 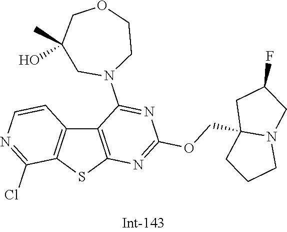 [SMILES: C[C@@]1(O)COCCN(c2nc(OC[C@@]34CCCN3C[C@H](F)C4)nc3sc4c(Cl)nccc4c23)C1] | 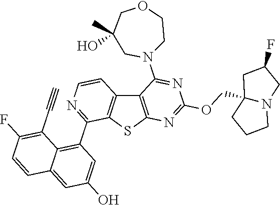 [SMILES: C#Cc1c(F)ccc2cc(O)cc(-c3nccc4c3sc3nc(OC[C@@]56CCCN5C[C@H](F)C6)nc(N5CCOC[C@@](C)(O)C5)c34)c12] | (S)-4-(8-(8-ethynyl- 7-fluoro-3-hydroxy- naphthalen-1-yl)-2- (((2R,7aS)-2-fluoro- tetrahydro-1H-pyr- rolizin-7a(5H)-yl)- methoxy)pyrido [4′,3′:4,5]thieno[2,3- d]pyrimidin-4-yl)-6- methyl-1,4-oxaze- pan-6-ol | 658 |
| Ex- 136 | 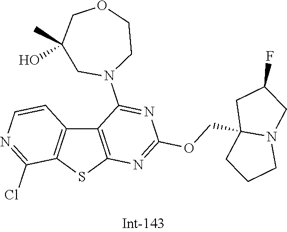 [SMILES: C[C@@]1(O)COCCN(c2nc(OC[C@@]34CCCN3C[C@H](F)C4)nc3sc4c(Cl)nccc4c23)C1] | 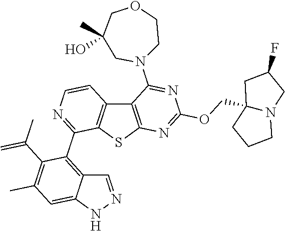 [SMILES: C=C(C)c1c(C)cc2[nH]ncc2c1-c1nccc2c1sc1nc(OC[C@@]34CCCN3C[C@H](F)C4)nc(N3CCOC[C@@](C)(O)C3)c12] | (6S)-4-(2-(((2R, 7aS)-2-fluorotetra- hydro-1H-pyrrol- izin-7a(5H)-yl)meth- oxy)-8-(6-methyl-5- (prop-1-en-2-yl)- 1H-indazol-4-yl)pyr- ido[4′,3′:4,5]thieno [2,3-d]pyrimidin-4- yl)-6-methyl-1,4- oxazepan-6-ol | 644 |
| Ex- 137 | 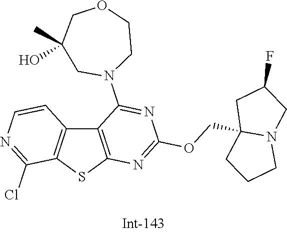 [SMILES: C[C@@]1(O)COCCN(c2nc(OC[C@@]34CCCN3C[C@H](F)C4)nc3sc4c(Cl)nccc4c23)C1] | 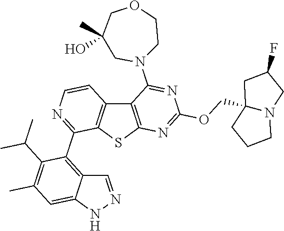 [SMILES: Cc1cc2[nH]ncc2c(-c2nccc3c2sc2nc(OC[C@@]45CCCN4C[C@H](F)C5)nc(N4CCOC[C@@](C)(O)C4)c23)c1C(C)C] | (6S)-4-(2-(((2R, 7aS)-2-fluorotetra- hydro-1H-pyrrol- izin-7a(5H)-yl)meth- oxy)-8-(5-isopropyl- 6-methyl-1H-ind- azol-4-yl)pyrido [4′,3′:4,5]thieno[2,3- d]pyrimidin-4-yl)-6- methyl-1,4-oxaze- pan-6-ol | 646 |
| Ex- 138 | 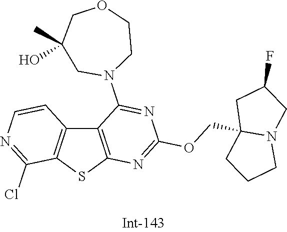 [SMILES: C[C@@]1(O)COCCN(c2nc(OC[C@@]34CCCN3C[C@H](F)C4)nc3sc4c(Cl)nccc4c23)C1] | 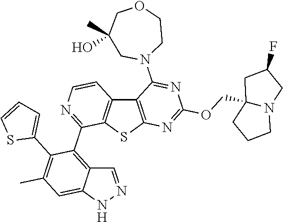 [SMILES: Cc1cc2[nH]ncc2c(-c2nccc3c2sc2nc(OC[C@@]45CCCN4C[C@H](F)C5)nc(N4CCOC[C@@](C)(O)C4)c23)c1-c1cccs1] | (6S)-4-(2-(((2R, 7aS)-2-fluorotetra- hydro-1H-pyrrolizin- 7a(5H)-yl)methoxy)- 8-(6-methyl-5-(thio- phen-2-yl)-1H-ind- azol-4-yl)pyrido [4′,3′:4,5]thieno[2,3- d]pyrimidin-4-yl)-6- methyl-1,4-oxaze- pan-6-ol | 686 |
| Ex- 139 | 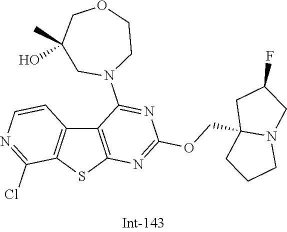 [SMILES: C[C@@]1(O)COCCN(c2nc(OC[C@@]34CCCN3C[C@H](F)C4)nc3sc4c(Cl)nccc4c23)C1] | 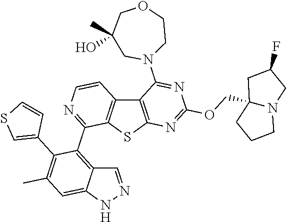 [SMILES: Cc1cc2[nH]ncc2c(-c2nccc3c2sc2nc(OC[C@@]45CCCN4C[C@H](F)C5)nc(N4CCOC[C@@](C)(O)C4)c23)c1-c1ccsc1] | (6S)-4-(2-(((2R, 7aS)-2-fluorotetra- hydro-1H-pyrrol- izin-7a(5H)-yl)meth- oxy)-8-(6-methyl-5- (thiophen-3-yl)-1H- indazol-4-yl)pyrido [4′,3′:4,5]thieno[2,3- d]pyrimidin-4-yl)-6- methyl-1,4-oxaze- pan-6-ol | 686 |
| Ex- 140 | 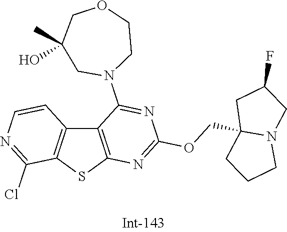 [SMILES: C[C@@]1(O)COCCN(c2nc(OC[C@@]34CCCN3C[C@H](F)C4)nc3sc4c(Cl)nccc4c23)C1] | 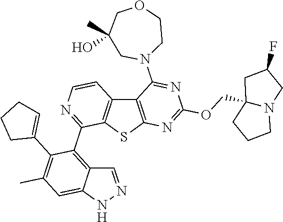 [SMILES: Cc1cc2[nH]ncc2c(-c2nccc3c2sc2nc(OC[C@@]45CCCN4C[C@H](F)C5)nc(N4CCOC[C@@](C)(O)C4)c23)c1C1=CCCC1] | (6S)-4-(8-(5-(cyclo- pent-1-en-1-yl)-6- methyl-1H-indazol- 4-yl)-2-(((2R,7aS)-2- fluorotetrahydro-1H- pyrrolizin-7a(5H)- yl)methoxy)pyrido [4′,3′:4,5]thieno[2,3- d]pyrimidin-4-yl)-6- methyl-1,4-oxaze- pan-6-ol | 670 |
| Ex- 141 | 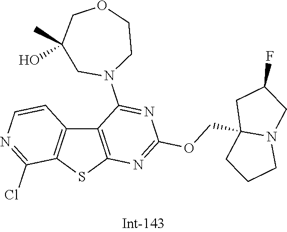 [SMILES: C[C@@]1(O)COCCN(c2nc(OC[C@@]34CCCN3C[C@H](F)C4)nc3sc4c(Cl)nccc4c23)C1] | 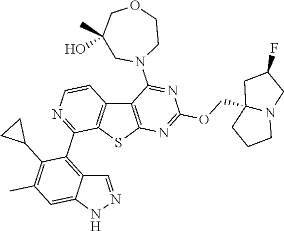 [SMILES: Cc1cc2[nH]ncc2c(-c2nccc3c2sc2nc(OC[C@@]45CCCN4C[C@H](F)C5)nc(N4CCOC[C@@](C)(O)C4)c23)c1C1CC1] | (6S)-4-(8-(5-cyclo- propyl-6-methyl- 1H-indazol-4-yl)-2- (((2R,7aS)-2-fluoro- tetrahydro-1H-pyr- rolizin-7a(5H)-yl)- methoxy)pyrido [4′,3′:4,5]thieno[2,3- d]pyrimidin-4-yl)-6- methyl-1,4-oxaze- pan-6-ol | 644 |
| Ex- 142 | 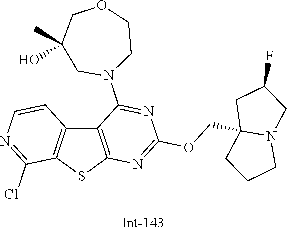 [SMILES: C[C@@]1(O)COCCN(c2nc(OC[C@@]34CCCN3C[C@H](F)C4)nc3sc4c(Cl)nccc4c23)C1] | 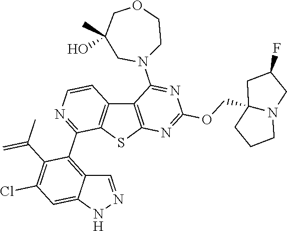 [SMILES: C=C(C)c1c(Cl)cc2[nH]ncc2c1-c1nccc2c1sc1nc(OC[C@@]34CCCN3C[C@H](F)C4)nc(N3CCOC[C@@](C)(O)C3)c12] | (6S)-4-(8-(6-chloro- 5-(prop-1-en-2-yl)- 1H-indazol-4-yl)-2- (((2R,7aS)-2-fluoro- tetrahydro-1H-pyr- rolizin-7a(5H)-yl)- methoxy)pyrido [4′,3′:4,5]thieno[2,3- d]pyrimidin-4-yl)-6- methyl-1,4-oxaze- pan-6-ol | 664 |
| Ex- 143 | 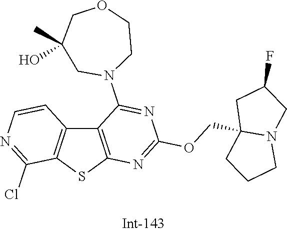 [SMILES: C[C@@]1(O)COCCN(c2nc(OC[C@@]34CCCN3C[C@H](F)C4)nc3sc4c(Cl)nccc4c23)C1] | 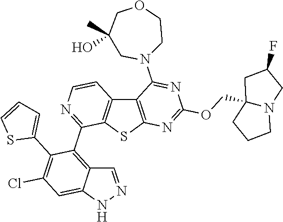 [SMILES: C[C@@]1(O)COCCN(c2nc(OC[C@@]34CCCN3C[C@H](F)C4)nc3sc4c(-c5c(-c6cccs6)c(Cl)cc6[nH]ncc56)nccc4c23)C1] | (6S)-4-(8-(6-chloro- 5-(thiophen-2-yl)- 1H-indazol-4-yl)-2- (((2R,7aS)-2-fluoro- tetrahydro-1H-pyr- rolizin-7a(5H)-yl)- methoxy)pyrido [4′,3′:4,5]thieno[2,3- d]pyrimidin-4-yl)-6- methyl-1,4-oxaze- pan-6-ol | 706 |
| Ex- 144 | 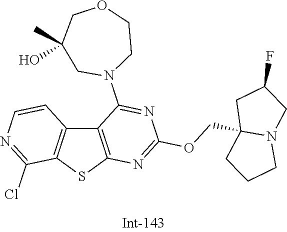 [SMILES: C[C@@]1(O)COCCN(c2nc(OC[C@@]34CCCN3C[C@H](F)C4)nc3sc4c(Cl)nccc4c23)C1] | 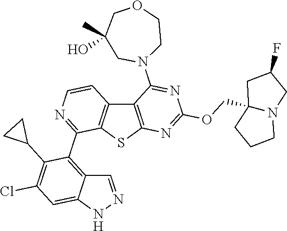 [SMILES: C[C@@]1(O)COCCN(c2nc(OC[C@@]34CCCN3C[C@H](F)C4)nc3sc4c(-c5c(C6CC6)c(Cl)cc6[nH]ncc56)nccc4c23)C1] | (6S)-4-(8-(6-chloro- 5-cyclopropyl-1H- indazol-4-yl)-2- (((2R,7aS)-2-fluoro- tetrahydro-1H-pyr- rolizin-7a(5H)-yl)- methoxy)pyrido [4′,3′:4,5]thieno[2,3- d]pyrimidin-4-yl)-6- methyl-1,4-oxaze- pan-6-ol | 664 |
| Ex- 145 | 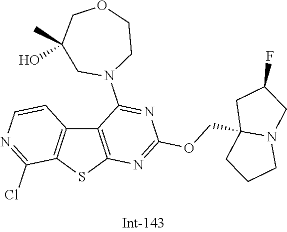 [SMILES: C[C@@]1(O)COCCN(c2nc(OC[C@@]34CCCN3C[C@H](F)C4)nc3sc4c(Cl)nccc4c23)C1] | 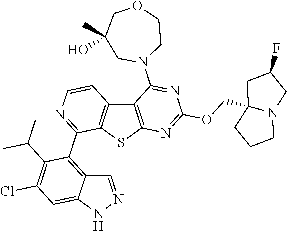 [SMILES: CC(C)c1c(Cl)cc2[nH]ncc2c1-c1nccc2c1sc1nc(OC[C@@]34CCCN3C[C@H](F)C4)nc(N3CCOC[C@@](C)(O)C3)c12] | (6S)-4-(8-(6-chloro- 5-isopropyl-1H-ind- azol-4-yl)-2-(((2R, 7aS)-2-fluorotetra- hydro-1H-pyrrol- izin-7a(5H)-yl)meth- oxy)pyrido[4′,3′:4,5] thieno[2,3-d]pyr- imidin-4-yl)-6-meth- yl-1,4-oxazepan-6-ol | 666 |
| Ex- 146 | 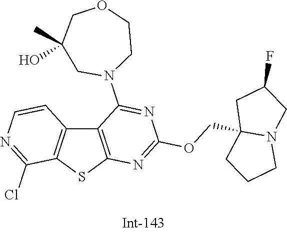 [SMILES: C[C@@]1(O)COCCN(c2nc(OC[C@@]34CCCN3C[C@H](F)C4)nc3sc4c(Cl)nccc4c23)C1] | 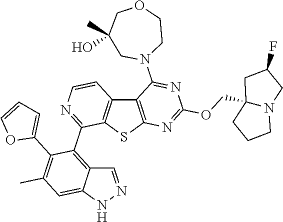 [SMILES: Cc1cc2[nH]ncc2c(-c2nccc3c2sc2nc(OC[C@@]45CCCN4C[C@H](F)C5)nc(N4CCOC[C@@](C)(O)C4)c23)c1-c1ccco1] | (6S)-4-(2-(((2R, 7aS)-2-fluorotetra- hydro-1H-pyrrol- izin-7a(5H)-yl)meth- oxy)-8-(5-(furan-2- yl)-6-methyl-1H- indazol-4-yl)pyrido [4′,3′:4,5]thieno[2,3- d]pyrimidin-4-yl)-6- methyl-1,4-oxaze- pan-6-ol | 670 |
| Ex- 147 | 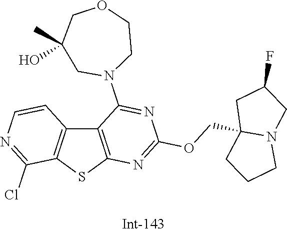 [SMILES: C[C@@]1(O)COCCN(c2nc(OC[C@@]34CCCN3C[C@H](F)C4)nc3sc4c(Cl)nccc4c23)C1] | 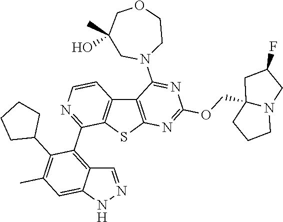 [SMILES: Cc1cc2[nH]ncc2c(-c2nccc3c2sc2nc(OC[C@@]45CCCN4C[C@H](F)C5)nc(N4CCOC[C@@](C)(O)C4)c23)c1C1CCCC1] | (6S)-4-(8-(5-cyclo- pentyl-6-methyl- 1H-indazol-4-yl)-2- (((2R,7aS)-2-fluoro- tetrahydro-1H-pyr- rolizin-7a(5H)-yl)- methoxy)pyrido [4′,3′:4,5]thieno[2,3- d]pyrimidin-4-yl)-6- methyl-1,4-oxaze- pan-6-ol | 672 |
| Ex- 148 | 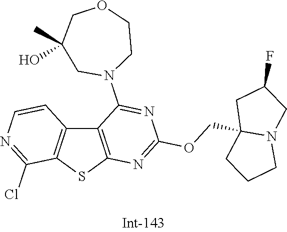 [SMILES: C[C@@]1(O)COCCN(c2nc(OC[C@@]34CCCN3C[C@H](F)C4)nc3sc4c(Cl)nccc4c23)C1] | 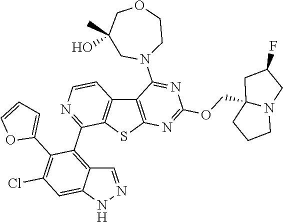 [SMILES: C[C@@]1(O)COCCN(c2nc(OC[C@@]34CCCN3C[C@H](F)C4)nc3sc4c(-c5c(-c6ccco6)c(Cl)cc6[nH]ncc56)nccc4c23)C1] | (6S)-4-(8-(6-chloro- 5-(furan-2-yl)-1H- indazol-4-yl)-2- (((2R,7aS)-2-fluoro- tetrahydro-1H-pyr- rolizin-7a(5H)-yl)- methoxy)pyrido [4′,3′:4,5]thieno[2,3- d]pyrimidin-4-yl)-6- methyl-1,4-oxaze- pan-6-ol | 690 |

### Example 149: (69)-4-(2-(((2R,7aS)-2-fluorotetrahydro-1H-pyrrolizin-7a(5H)-yl)methoxy)-8-(3-iodo-6-methyl-5-(trifluoromethyl)-1H-indazol-4-yl)pyrido[4′,3′:4,5]thieno[2,3-d]pyrimidin-4-yl)-6-methyl-1,4-oxazepan-6-ol (Ex-149)

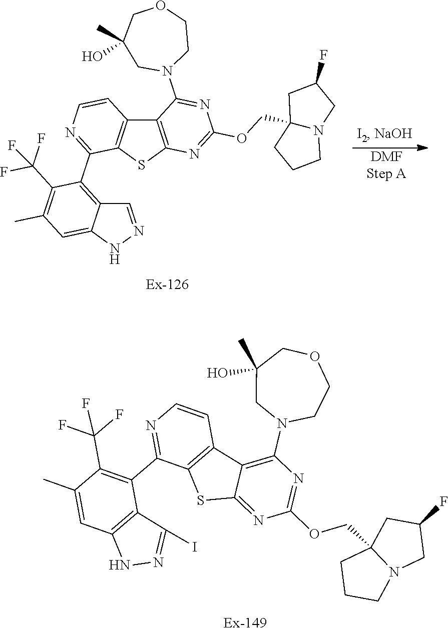 [SMILES: Cc1cc2[nH]ncc2c(-c2nccc3c2sc2nc(OC[C@@]45CCCN4C[C@H](F)C5)nc(N4CCOC[C@@](C)(O)C4)c23)c1C(F)(F)F ; Cc1cc2[nH]nc(I)c2c(-c2nccc3c2sc2nc(OC[C@@]45CCCN4C[C@H](F)C5)nc(N4CCOC[C@@](C)(O)C4)c23)c1C(F)(F)F]

To a stirred mixture of (6S)-4-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidin-4-yl)-6-methyl-1,4-oxazepan-6-ol (Ex-126) (55 mg, 0.082 mmol) and sodium hydroxide (10 mg, 0.25 mmol) in DMF was added iodine (83 mg, 0.33 mmol) at r.t. After being stirred for 1.5 h, the mixture was quenched by the addition of 10% aq. sodium thiosulfate solution. The mixture was extracted with EtOAc and the organic phase was washed with water and brine, dried over sodium sulfate, filtered, and concentrated. The crude residue was purified via silica gel chromatography (0 to 25% MeOH in chloroform) to give (6S)-4-(2-(((2R,7aS)-2-fluorotetrahydro-1H-pyrrolizin-7a(5H)-yl)methoxy)-8-(3-iodo-6-methyl-5-(trifluoromethyl)-1H-indazol-4-yl)pyrido[4′,3′:4,5]thieno[2,3-d]pyrimidin-4-yl)-6-methyl-1,4-oxazepan-6-ol (Ex-149). MS (ESI): m/z (M+H)⁺ 798. ¹H NMR (400 MHz, CDCl₃) δ 11.50-10.80 (brs, 1H), 8.80-8.76 (m, 1H), 7.65-7.62 (m, 1H), 7.55-7.54 (brs, 1H), 6.72-6.42 (m, 1H), 5.36-5.22 (m, 1H), 4.31-4.03 (m, 2H), 3.99-3.89 (m, 1H), 3.82-3.70 (m, 4H), 3.69-3.61 (m, 3H), 3.45-3.17 (m, 3H), 3.04-2.95 (m, 1H), 2.71-2.70 (brs, 3H), 2.24-1.95 (m, 6H), 1.38 (s, 1.5H), 1.36 (s, 1.5H).

### Example 150; 4-(2-(((2R,7aS)-2-fluorotetrahydro-1H-pyrrolizin-7a(5H)-yl)methoxy)-4-((S)-6-hydroxy-6-methyl-1,4-oxazepan-4-yl)pyrido[4′,3′:4,5]thieno[2,3-d]pyrimidin-8-yl)-6-methyl-5-(trifluoromethyl)-1H-indazole-3-carbonitrile (Ex-150)

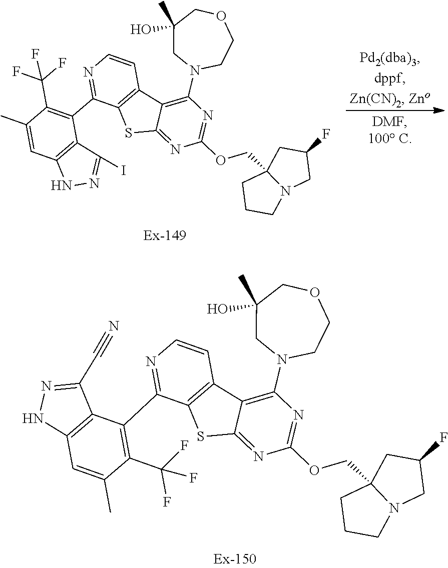 [SMILES: Cc1cc2[nH]nc(I)c2c(-c2nccc3c2sc2nc(OC[C@@]45CCCN4C[C@H](F)C5)nc(N4CCOC[C@@](C)(O)C4)c23)c1C(F)(F)F ; Cc1cc2[nH]nc(C#N)c2c(-c2nccc3c2sc2nc(OC[C@@]45CCCN4C[C@H](F)C5)nc(N4CCOC[C@@](C)(O)C4)c23)c1C(F)(F)F]

A mixture of (6S)-4-(2-(((2R,7aS)-2-fluorotetrahydro-1H-pyrrolizin-7a(5H)-yl)methoxy)-8-(3-iodo-6-methyl-5-(trifluoromethyl)-1H-indazol-4-yl)pyrido[4′,3′:4,5]thieno[2,3-d]pyrimidin-4-yl)-6-methyl-1,4-oxazepan-6-ol (Ex-149) (32 mg, 0.040 mmol), zinc (5 mg, 0.080 mmol), zinc cyanide (14 mg, 0.12 mmol), 1,1′-bis(diphenylphosphino)ferrocene (9 mg, 0.02 mmol), and tris(dibenzylideneacetone) dipalladium (0) (7 mg, 0.008 mmol) in DMF (2 ml) was stirred at 100° C. for 1.5 h under nitrogen atmosphere. After cooling, the insoluble materials were filtered by a CELITE® pad and the filtrate was partitioned between EtOAc and water. The organic phase was washed with water and brine, dried over sodium sulfate, filtered, and concentrated. The crude residue was purified via preparative reverse-phase HPLC (MeCN/H₂O w/formic acid as modifier) to give 4-(2-(((2R,7aS)-2-fluorotetrahydro-1H-pyrrolizin-7a(5H)-yl)methoxy)-4-((S)-6-hydroxy-6-methyl-1,4-oxazepan-4-yl)pyrido[4′,3′:4,5]thieno[2,3-d]pyrimidin-8-yl)-6-methyl-5-(trifluoromethyl)-1H-indazole-3-carbonitrile (Ex-150). MS (ESI): m/z (M+H)⁺ 697. ¹H NMR (400 MHz, CDCl₃) δ=8.83-8.72 (m, 1H), 7.71-7.65 (m, 1H), 7.59-7.52 (m, 1H), 5.39-5.15 (m, 1H), 4.30-4.04 (m, 4H), 3.98-3.85 (m, 1H), 3.83-3.55 (m, 4H), 3.40-3.12 (m, 5H), 3.07-2.97 (m, 1H), 2.74-2.68 (m, 3H), 2.32-2.19 (m, 1H), 2.17-2.06 (m, 2H), 2.06-1.90 (m, 2H), 1.35 (s, 3H).

### Example 151: (6S)-4-(8-(3-ethynyl-6-methyl-5-(trifluoromethyl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-151)

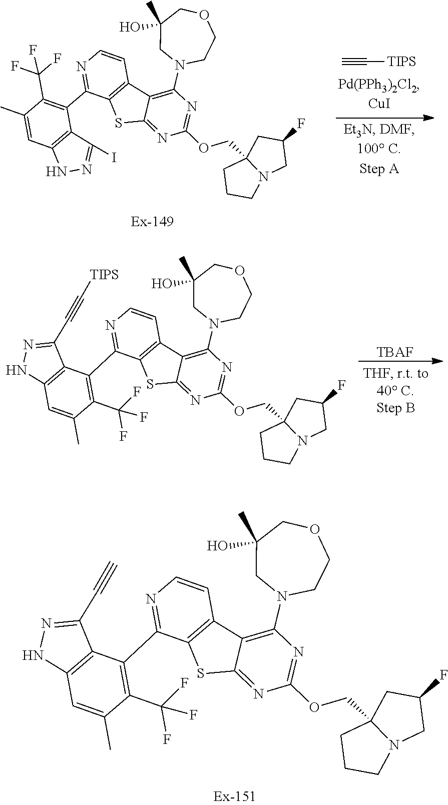

### Step (6S)-4-(2-(((2R,7aS)-2-fluorotetrahydro-1H-pyrrolizin-7a(5H)-yl)methoxy)-8-(6-methyl-5-(trifluoromethyl)-3-((triisopropylsilyl)ethynyl)-1H-indazol-4-yl)pyrido[4′,3′:4,5]thieno[2,3-d]pyrimidin-4-yl)-6-methyl-1,4-oxazepan-6-ol

A mixture of (6S)-4-(2-(((2R,7aS)-2-fluorotetrahydro-1H-pyrrolizin-7a(5H)-yl)methoxy)-8-(3-iodo-6-methyl-5-(trifluoromethyl)-1H-indazol-4-yl)pyrido[4′,3′:4,5]thieno[2,3-d]pyrimidin-4-yl)-6-methyl-1,4-oxazepan-6-ol (Ex-149) (13 mg, 0.016 mmol), copper iodide (0.9 mg, 0.049 mmol), ethynyltriisopropylsilane (0.022 ml, 0.098 mmol), bis(triphenylphosphine) palladium (II) dichloride (3.4 mg, 0.049 mmol), and triethylamine (100 μl) in DMF (0.3 ml) was heated at 100° C. for 2 h. After cooling, the mixture was partitioned between EtOAc and water. The organic phase was washed with water and brine, dried over sodium sulfate, filtered and concentrated to give crude (6S)-4-(2-(((2R,7aS)-2-fluorotetrahydro-1H-pyrrolizin-7a(5H)-yl)methoxy)-8-(6-methyl-5-(trifluoromethyl)-3-((triisopropylsilyl)ethynyl)-1H-indazol-4-yl)pyrido[4′,3′:4,5]thieno[2,3-d]pyrimidin-4-yl)-6-methyl-1,4-oxazepan-6-ol which was used for next reaction without further purification. MS (ESI): m/z (M+H)⁺ 852.

### Step B: (6S)-4-(8-(3-ethynyl-6-methyl-5-(trifluoromethyl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-151)

To a stirred solution of (6S)-4-(2-(((2R,7aS)-2-fluorotetrahydro-1H-pyrrolizin-7a(5H)-yl)methoxy)-8-(6-methyl-5-(trifluoromethyl)-3-((triisopropylsilyl)ethynyl)-1H-indazol-4-yl)pyrido[4′,3′:4,5]thieno[2,3-d]pyrimidin-4-yl)-6-methyl-1,4-oxazepan-6-ol in THF (0.5 ml) was added TBAF (50 μl, 0.05 mmol) at r.t. Additional TBAF (50 μl, 0.05 mmol) was added twice at 1 h and at 1.5 h then the mixture was stirred at 40° C. for another 0.5 h. The mixture was partitioned between EtOAc and water. The organic phase was washed with water and brine, dried over sodium sulfate, filtered and concentrated. The crude residue was purified via reverse phase HPLC (MeCN/H₂O with formic acid as modifier) to give (6S)-4-(8-(3-ethynyl-6-methyl-5-(trifluoromethyl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-151). MS (ESI): m/z (M+H)⁺ 696, ¹H NMR (400 MHz, CDCl₃) δ 8.73-8.70 (m, 1H), 7.61-7.58 (m, 1H), 7.55-7.54 (brs, 1H), 6.43 (brm, 1H), 5.35-5.22 (m, 1H), 4.31-4.05 (m, 4H), 3.98-3.89 (m, 1H), 3.82-3.71 (m, 2H), 3.69-3.61 (m, 2H), 3.47-3.15 (m, 5H), 3.02-2.94 (m, 1H), 2.74-2.70 (brs, 3H), 2.40-2.03 (m, 3H), 2.00-1.90 (m, 3H), 1.365 (m, 3H).

### Example 152: (6S)-4-(8-(5-(1,1-difluoroallyl)-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-152)

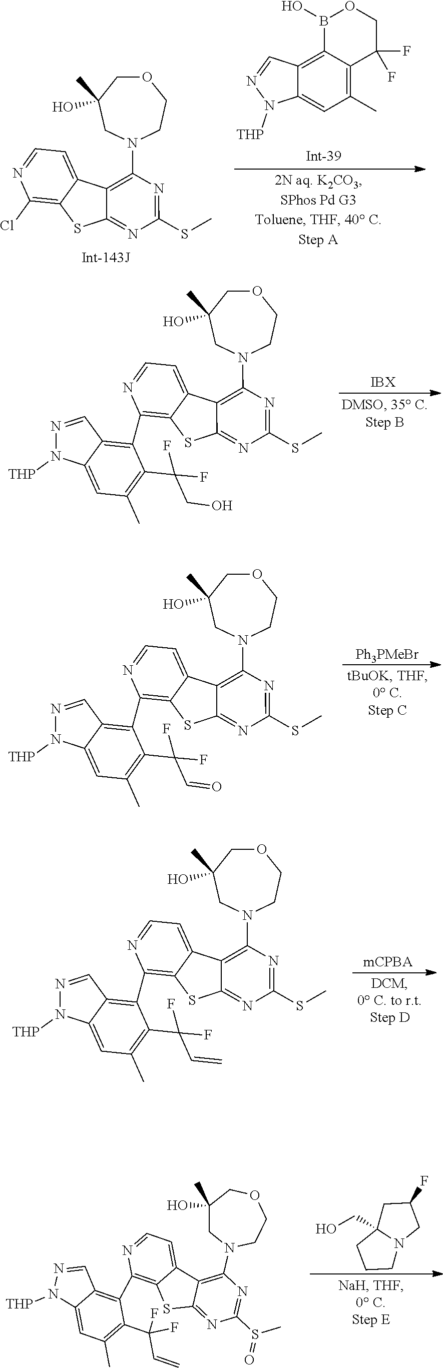

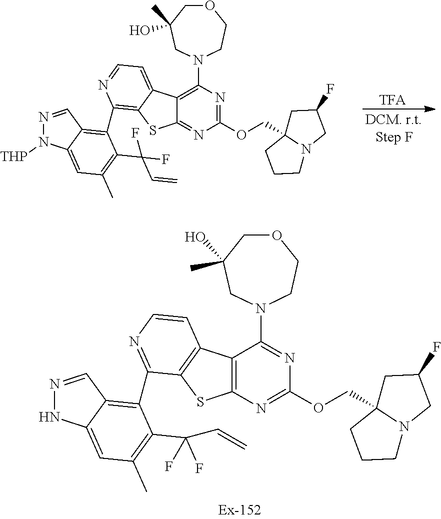

### Step A: (6R)-4-(8-(5-(1,1-difluoro-2-hydroxyethyl)-6-methyl-1-(tetrahydro-2H-pyran-2-yl)-1H-indazol-4-yl)-2-(methylthio)pyrido[4′,3′:4,5]thieno[2,3-d]pyrimidin-4-yl)-6-methyl-1,4-oxazepan-6-ol

To a solution of(S)-4-(8-chloro-2-(methylthio)pyrido[4′,3′:4,5]thieno[2,3-d]pyrimidin-4-yl)-6-methyl-1,4-oxazepan-6-ol (Int-143J) (300 mg, 0.756 mmol), 4,4-difluoro-5-methyl-7-(tetrahydro-2H-pyran-2-yl)-4,7-dihydro-[1,2]oxaborinino[3,4-e]indazol-1 (3H)-ol (Int-39) (292 mg, 0.907 mmol) in THF (3 mL) and toluene (3 mL) was added aq. K₂CO₃ (1.134 mL, 2.268 mmol, 2 M in H₂O) and SPhos Pd G3 (295 mg, 0.378 mmol) at 25° C. under N₂. The mixture was stirred at 40° C. for 1 h. The mixture was cooled, diluted with water (5 mL), extracted with EtOAc (3×5 mL), dried over sodium sulfate, filtered, and the solvent was evaporated under reduced pressure to give the crude product. The crude product was purified by flash silica gel chromatography (0 to 50% EtOAc in petroleum ether) to give (6R)-4-(8-(5-(1,1-difluoro-2-hydroxyethyl)-6-methyl-1-(tetrahydro-2H-pyran-2-yl)-1H-indazol-4-yl)-2-(methylthio)pyrido[4′,3′:4,5]thieno[2,3-d]pyrimidin-4-yl)-6-methyl-1,4-oxazepan-6-ol. MS (ESI) [M+H]⁺: m/z 657.

### Step B: 2,2-difluoro-2-(4-(4-((S)-6-hydroxy-6-methyl-1,4-oxazepan-4-yl)-2-(methylthio)pyrido[4′,3′:4,5]thieno[2,3-d]pyrimidin-8-yl)-6-methyl-1-(tetrahydro-2H-pyran-2-yl)-1H-indazol-5-yl) acetaldehyde

To a solution of (6R)-4-(8-(5-(1,1-difluoro-2-hydroxyethyl)-6-methyl-1-(tetrahydro-2H-pyran-2-yl)-1H-indazol-4-yl)-2-(methylthio)pyrido[4′,3′:4,5]thieno[2,3-d]pyrimidin-4-yl)-6-methyl-1,4-oxazepan-6-ol (220 mg, 0.335 mmol) in DMSO (3 mL) was added 2-iodoxybenzoic acid (234 mg, 0.837 mmol) at 25° C. The mixture was stirred at 35° C. for 12 h. The mixture was quenched with sat. aq. NaHCO₃ (1.5 mL), diluted with EtOAc (30 mL), and filtered. The organic layer was separated and washed with brine (3×3 mL), dried over sodium sulfate, filtered, and the solvent was evaporated under reduced pressure to give the crude product. The crude product was purified by preparative TLC (SiO₂, DCM:MeOH=15:1) to give 2,2-difluoro-2-(4-(4-((S)-6-hydroxy-6-methyl-1,4-oxazepan-4-yl)-2-(methylthio)pyrido[4′,3′:4,5]thieno[2,3-d]pyrimidin-8-yl)-6-methyl-1-(tetrahydro-2H-pyran-2-yl)-1H-indazol-5-yl) acetaldehyde. MS (ESI) [M+H]⁺: m/z 655.

### Step C: (6S)-4-(8-(5-(1,1-difluoroallyl)-6-methyl-1-(tetrahydro-2H-pyran-2-yl)-1H-indazol-4-yl)-2-(methylthio)pyrido[4′,3′:4,5]thieno[2,3-d]pyrimidin-4-yl)-6-methyl-1,4-oxazepan-6-ol

To a solution of methyltriphenylphosphonium bromide (85 mg, 0.238 mmol) in THF (1 mL) was added/BuOK (0.238 mL, 0.238 mmol, 1 M in THF) at 0° C. under N₂. The mixture was stirred at 0° C. for 10 min, then 2,2-difluoro-2-(4-(4-((S)-6-hydroxy-6-methyl-1,4-oxazepan-4-yl)-2-(methylthio)pyrido[4′,3′:4,5]thieno[2,3-d]pyrimidin-8-yl)-6-methyl-1-(tetrahydro-2H-pyran-2-yl)-1H-indazol-5-yl) acetaldehyde (80 mg, 0.079 mmol) was added. The mixture was stirred at 0° C. for another 20 min. The mixture was quenched with sat. aq. NH₄Cl (2 mL), extracted with EtOAc (3×2 mL), dried over sodium sulfate, filtered, and the solvent was evaporated under reduced pressure. The residue was purified by preparative TLC (SiO₂, ethyl acetate) to give (6S)-4-(8-(5-(1,1-difluoroallyl)-6-methyl-1-(tetrahydro-2H-pyran-2-yl)-1H-indazol-4-yl)-2-(methylthio)pyrido[4′,3′:4,5]thieno[2,3-d]pyrimidin-4-yl)-6-methyl-1,4-oxazepan-6-ol. MS (ESI) [M+H]⁺: m/z 653.

### Step D: (6S)-4-(8-(5-(1,1-difluoroallyl)-6-methyl-1-(tetrahydro-2H-pyran-2-yl)-1H-indazol-4-yl)-2-(methylsulfinyl)pyrido[4′,3′:4,5]thieno[2,3-d]pyrimidin-4-yl)-6-methyl-1,4-oxazepan-6-ol

To a solution of (6S)-4-(8-(5-(1,1-difluoroallyl)-6-methyl-1-(tetrahydro-2H-pyran-2-yl)-1H-indazol-4-yl)-2-(methylthio)pyrido[4′,3′:4,5]thieno[2,3-d]pyrimidin-4-yl)-6-methyl-1,4-oxazepan-6-ol (15 mg, 0.023 mmol) in DCM (0.2 mL) was added mCPBA (11.66 mg, 0.057 mmol) at 0° C. The mixture was warmed to r.t. stirred for 30 min. The mixture was quenched with sat. aq. NaHCO₃ (1 mL), extracted with DCM (3×1 mL), dried over sodium sulfate, filtered, and the solvent was evaporated under reduced pressure to give the crude product. The residue was purified by preparative TLC (SiO₂, EtOAc) to give (6S)-4-(8-(5-(1,1-difluoroallyl)-6-methyl-1-(tetrahydro-2H-pyran-2-yl)-1H-indazol-4-yl)-2-(methylsulfinyl)pyrido[4′,3′:4,5]thieno[2,3-d]pyrimidin-4-yl)-6-methyl-1,4-oxazepan-6-ol. MS (ESI) [M+H]⁺: m/z 669.

### Step E: (6R)-4-(8-(5-(1,1-difluoroallyl)-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol

To a solution of ((2R,7aS)-2-fluorotetrahydro-1H-pyrrolizin-7a(5H)-yl) methanol (1.785 mg, 0.011 mmol) in THF (0.2 mL) was added sodium hydride (0.598 mg, 0.015 mmol, 60% in mineral oil) at 0° C. under nitrogen atmosphere. The mixture was stirred at 0° C. for 5 min, and then (6S)-4-(8-(5-(1,1-difluoroallyl)-6-methyl-1-(tetrahydro-2H-pyran-2-yl)-1H-indazol-4-yl)-2-(methylsulfinyl)pyrido[4′,3′:4,5]thieno[2,3-d]pyrimidin-4-yl)-6-methyl-1,4-oxazepan-6-ol (5 mg, 7.48 μmol) was added. The mixture was stirred at 0° C. for another 20 min. The mixture was quenched with sat. aq. NH₄Cl (1 mL), and extracted with DCM (3×1 mL). The organic layer was dried over sodium sulfate, filtered, and the solvent was evaporated under reduced pressure to give the crude product. The residue was purified by preparative TLC (SiO₂, DCM:MeOH=10:1) to give (6R)-4-(8-(5-(1,1-difluoroallyl)-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol. MS (ESI) [M+H]⁺: m/z 764.

### Step F: (6S)-4-(8-(5-(1,1-difluoroallyl)-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-152)

To a solution of (6S)-4-(8-(5-(1,1-difluoroallyl)-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (6 mg, 7.85 μmol) in DCM (0.1 mL) was added TFA (0.1 mL, 1.298 mmol) at 0° C. The mixture was stirred at 25° C. for 10 min. The mixture was evaporated under reduced pressure to give the crude product. The residue was purified by preparative reverse-phase HPLC (MeCN/H₂O w/TFA modifier) to give (6S)-4-(8-(5-(1,1-difluoroallyl)-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-152) as the TFA salt. MS (ESI) [M+H]⁺: m/z 680. ¹H NMR (400 MHz, CD₃OD) δ 8.73 (d, J=5.8 Hz, 1H), 7.98 (d, J=5.7 Hz, 1H), 7.71 (s, 1H), 7.38 (d, J=2.3 Hz, 1H), 6.36-6.17 (m, 1H), 5.69-5.49 (m, 3H), 4.74-4.53 (m, 2H), 4.27-4.07 (m, 3H), 4.02-3.86 (m, 6H), 3.76-3.69 (m, 1H), 3.57-3.43 (m, 2H), 2.77-2.58 (m, 5H), 2.44-2.29 (m, 3H), 2.18 (brd, J=3.6 Hz, 1H), 1.21 (d, J=10.3 Hz, 3H).

### Example 153: (6S)-4-(8-(5-(1,1-difluoroprop-2-yn-1-yl)-6-methyl-1H-indazol-4-yl)-2-(((2R,7aS)-2-fluorotetrahydro-1H-pyrrolizin-7a(5H)-yl) methoxy)pyrido[4′,3′:4,5]thieno[2,3-d]pyrimidin-4-yl)-6-methyl-1,4-oxazepan-6-ol (Ex-153)

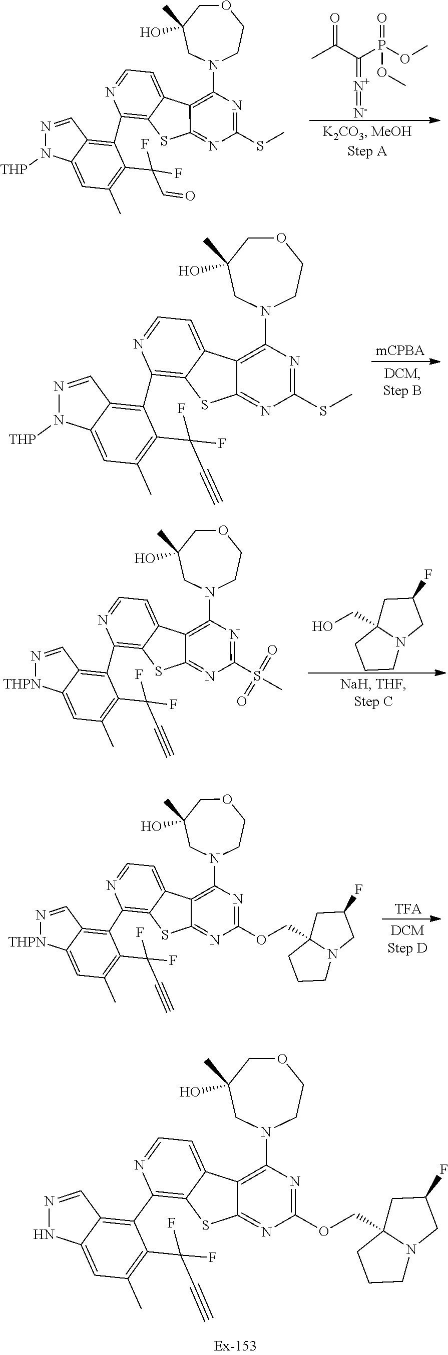

### Step A: (6S)-4-(8-(5-(1,1-difluoroprop-2-yn-1-yl)-6-methyl-1-(tetrahydro-2H-pyran-2-yl)-1H-indazol-4-yl)-2-(methylthio)pyrido[4′,3′:4,5]thieno[2,3-d]pyrimidin-4-yl)-6-methyl-1,4-oxazepan-6-ol

To a solution of 2,2-difluoro-2-(4-(4-((S)-6-hydroxy-6-methyl-1,4-oxazepan-4-yl)-2-(methylthio)pyrido[4′,3′:4,5]thieno[2,3-d]pyrimidin-8-yl)-6-methyl-1-(tetrahydro-2H-pyran-2-yl)-1H-indazol-5-yl) acetaldehyde (150 mg, 0.183 mmol) in MeOH (2 mL) was added potassium carbonate (50.7 mg, 0.367 mmol) and dimethyl(1-diazo-2-oxopropyl)phosphonate (52.8 mg, 0.275 mmol) at 25° C. under N₂. The mixture was stirred at 25° C. for 1 h. The reaction mixture was purified by preparative TLC directly (SiO₂, DCM:MeOH=15:1) to give (6S)-4-(8-(5-(1,1-difluoroprop-2-yn-1-yl)-6-methyl-1-(tetrahydro-2H-pyran-2-yl)-1H-indazol-4-yl)-2-(methylthio)pyrido[4′,3′:4,5]thieno[2,3-d]pyrimidin-4-yl)-6-methyl-1,4-oxazepan-6-ol. MS (ESI) [M+H]⁺: m/z 651.

### Step B: (6S)-4-(8-(5-(1,1-difluoroprop-2-yn-1-yl)-6-methyl-1-(tetrahydro-2H-pyran-2-yl)-1H-indazol-4-yl)-2-(methylsulfonyl)pyrido[4′,3′:4,5]thieno[2,3-d]pyrimidin-4-yl)-6-methyl-1,4-oxazepan-6-ol

To a solution of (6S)-4-(8-(5-(1,1-difluoroprop-2-yn-1-yl)-6-methyl-1-(tetrahydro-2H-pyran-2-yl)-1H-indazol-4-yl)-2-(methylthio)pyrido[4′,3′:4,5]thieno[2,3-d]pyrimidin-4-yl)-6-methyl-1,4-oxazepan-6-ol (42 mg, 0.065 mmol) in DCM (1 mL) was added mCPBA (32.8 mg, 0.161 mmol, 85% w/w) at 25° C. The mixture was stirred at 25° C. for 30 min. The reaction was purified by preparative TLC (SiO₂, DCM:MeOH=15:1) directly to give (6S)-4-(8-(5-(1,1-difluoroprop-2-yn-1-yl)-6-methyl-1-(tetrahydro-2H-pyran-2-yl)-1H-indazol-4-yl)-2-(methylsulfonyl)pyrido[4′,3′:4,5]thieno[2,3-d]pyrimidin-4-yl)-6-methyl-1,4-oxazepan-6-ol. MS (ESI) [M+H]⁺: m/z 683.

### Step C: (6R)-4-(8-(5-(1,1-difluoroprop-2-yn-1-yl)-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepam-6-ol

To a solution of (6S)-4-(8-(5-(1,1-difluoroprop-2-yn-1-yl)-6-methyl-1-(tetrahydro-2H-pyran-2-yl)-1H-indazol-4-yl)-2-(methylsulfonyl)pyrido[4′,3′:4,5]thieno[2,3-d]pyrimidin-4-yl)-6-methyl-1,4-oxazepan-6-ol (20 mg, 0.030 mmol) and ((2R,7aS)-2-fluorotetrahydro-1H-pyrrolizin-7a(5H)-yl) methanol (14.33 mg, 0.090 mmol) in THF (1 mL) was added sodium hydride (4.80 mg, 0.120 mmol, 60% in mineral oil) at 25° C. under N₂. The mixture was stirred at 25° C. for 15 min. The reaction was quenched with sat. aq. NH₄Cl (0.5 mL) and extracted with EtOAc (5 mL). The organic layer was dried over sodium sulfate, filtered, and concentrated in vacuo. The residue was purified by preparative TLC (SiO₂, DCM:MeOH=10:1) to give (6R)-4-(8-(5-(1,1-difluoroprop-2-yn-1-yl)-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol. MS (ESI) [M+H]⁺: m/z 763.

### Step D: (6S)-4-(8-(5-(1,1-difluoroprop-2-yn-1-yl)-6-methyl-1H-indazol-4-yl)-2-(((2R,7aS)-2-fluorotetrahydro-1H-pyrrolizin-7a(5H)-yl) methoxy)pyrido[4′,3′:4,5]thieno[2,3-d]pyrimidin-4-yl)-6-methyl-1,4-oxazepan-6-ol (Ex-153)

To a solution of (6R)-4-(8-(5-(1,1-difluoroprop-2-yn-1-yl)-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (16 mg, 0.021 mmol) in DCM (0.2 mL) was added TFA (0.2 mL) at 0° C. under N₂. The mixture was stirred at 0° C. for 30 min. The reaction was concentrated in vacuo. The residue was purified by preparative reverse-phase HPLC (MeCN/H₂O w/TFA modifier) to give (6S)-4-(8-(5-(1,1-difluoroprop-2-yn-1-yl)-6-methyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-153). MS (ESI) [M+H]⁺: m/z 678. ¹H NMR (400 MHz, CD₃OD) δ 8.71 (dd, J=3.58, 5.48 Hz, 1H), 7.75-7.80 (m, 1H), 7.69 (s, 1H), 7.28-7.39 (m, 1H), 5.41-5.59 (m, 1H), 4.52-4.59 (m, 2H), 4.07-4.16 (m, 1H), 4.01 (brd, J=14.78 Hz, 1H), 3.80-3.93 (m, 4H), 3.70-3.80 (m, 3H), 3.64-3.68 (m, 1H), 3.58-3.64 (m, 2H), 3.23-3.47 (m, 4H), 2.77 (s, 3H), 2.60-2.69 (m, 1H), 2.57 (br d, J=4.29 Hz, 1H), 2.47-2.49 (m, 1H), 1.18 (d, J=3.58 Hz, 3H).

### Example 154:2-(4-(2-(((2R,7aS)-2-fluorotetrahydro-1H-pyrrolizin-7a(5H)-yl)methoxy)-4-((S)-6-hydroxy-6-methyl-1,4-oxazepan-4-yl)pyrido[4′,3′:4,5]thieno[2,3-d]pyrimidin-8-yl)-6-methyl-1H-indazol-5-yl)-2-methylpropanenitrile (Ex-154)

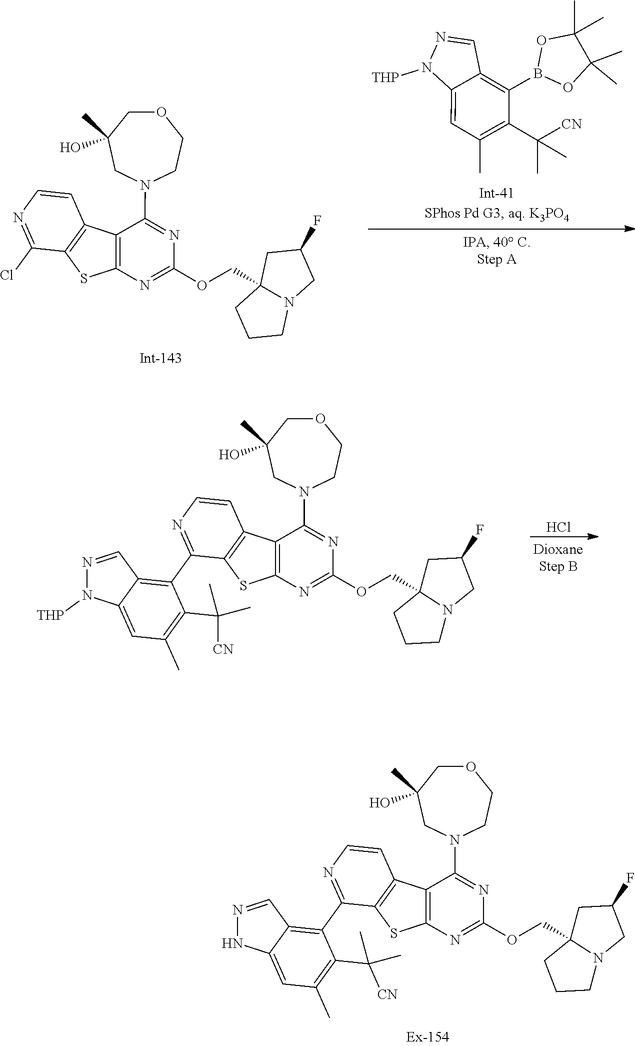

### Step A: 2-(4-(2-(((2R,7aS)-2-fluorotetrahydro-1H-pyrrolizin-7a(5H)-yl)methoxy)-4-((S)-6-hydroxy-6-methyl-1,4-oxazepan-4-yl)pyrido[4′,3′:4,5]thieno[2,3-d]pyrimidin-8-yl)-6-methyl-1-(tetrahydro-2H-pyran-2-yl)-1H-indazol-5-yl)-2-methylpropanenitrile

To a solution of(S)-4-(8-chloro-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Int-143) (330 mg, 0.650 mmol) in i-PrOH (3 mL) was added 2-methyl-2-(6-methyl-1-(tetrahydro-2H-pyran-2-yl)-4-(4,4,5,5˜tetramethyl-1,3,2-dioxaborolan-2-yl)-1H-indazol-5-yl) propanenitrile (Int-41) (220 mg, 0.537 mmol), K₃PO₄ (1.075 mL, 1.075 mmol, 1M in H₂O) and SPhos Pd G3 (84 mg, 0.107 mmol) at 25° C. under N₂. The mixture was stirred at 40° C. for 12 h. The mixture was cooled, diluted with water (3 mL), extracted with EtOAc (3×3 mL), dried over sodium sulfate, filtered, and the solvent was evaporated under reduced pressure. The residue was purified by preparative reverse-phase HPLC (MeCN/H₂O w/formic acid modifier) to give 2-(4-(2-(((2R,7aS)-2-fluorotetrahydro-1H-pyrrolizin-7a(5H)-yl)methoxy)-4-((S)-6-hydroxy-6-methyl-1,4-oxazepan-4-yl)pyrido[4′,3′:4,5]thieno[2,3-d]pyrimidin-8-yl)-6-methyl-1-(tetrahydro-2H-pyran-2-yl)-1H-indazol-5-yl)-2-methylpropanenitrile. MS (ESI) [M+H]⁺: m/z 756.

### Step B: 2-(4-(2-(((2R,7aS)-2-fluorotetrahydro-1H-pyrrolizin-7a(5H)-yl)methoxy)-4-((S)-6-hydroxy-6-methyl-1,4-oxazepan-4-yl)pyrido[4′,3′:4,5]thieno[2,3-d]pyrimidin-8-yl)-6-methyl-1H-indazol-5-yl)-2-methylpropanenitrile (Ex-154)

To a solution of 2-(4-(2-(((2R,7aS)-2-fluorotetrahydro-1H-pyrrolizin-7a(5H)-yl)methoxy)-4-((S)-6-hydroxy-6-methyl-1,4-oxazepan-4-yl)pyrido[4′,3′:4,5]thieno[2,3-d]pyrimidin-8-yl)-6-methyl-1-(tetrahydro-2H-pyran-2-yl)-1H-indazol-5-yl)-2-methylpropanenitrile (18 mg, 0.024 mmol) in i-PrOH (0.1 mL) was added HCl (0.1 mL, 0.400 mmol, 4 M in dioxane) at 25° C. The mixture was stirred at 25° C. for 30 min. The mixture was evaporated under reduced pressure. The residue was purified by preparative reverse-phase HPLC (MeCN/H₂O w/formic acid modifier) to give 2-(4-(2-(((2R,7aS)-2-fluorotetrahydro-1H-pyrrolizin-7a(5H)-yl)methoxy)-4-((S)-6-hydroxy-6-methyl-1,4-oxazepan-4-yl)pyrido[4′,3′:4,5]thieno[2,3-d]pyrimidin-8-yl)-6-methyl-1H-indazol-5-yl)-2-methylpropanenitrile (Ex-154). MS (ESI) [M+H]⁺: m/z 671. ¹H NMR (400 MHz, CD₃OD) δ 8.78-8.66 (m, 1H), 7.93 (d, J=5.6 Hz, 1H), 7.70 (s, 1H), 7.22 (d, J=13.0 Hz, 1H), 5.43-5.23 (m, 1H), 4.39-4.31 (m, 1H), 4.30-4.17 (m, 2H), 4.16-3.87 (m, 5H), 3.85-3.65 (m, 2H), 3.49 (t, J=12.9 Hz, 1H), 3.24 (br s, 2H), 3.13-3.00 (m, 1H), 2.94 (s, 3H), 2.28-1.88 (m, 6H), 1.77 (s, 3H), 1.54 (d, J=15.4 Hz, 3H), 1.21 (d, J=16.6 Hz, 3H).

### Example 155: (6S)-4-(8-(6-(difluoromethyl)-5-(trifluoromethyl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-155)

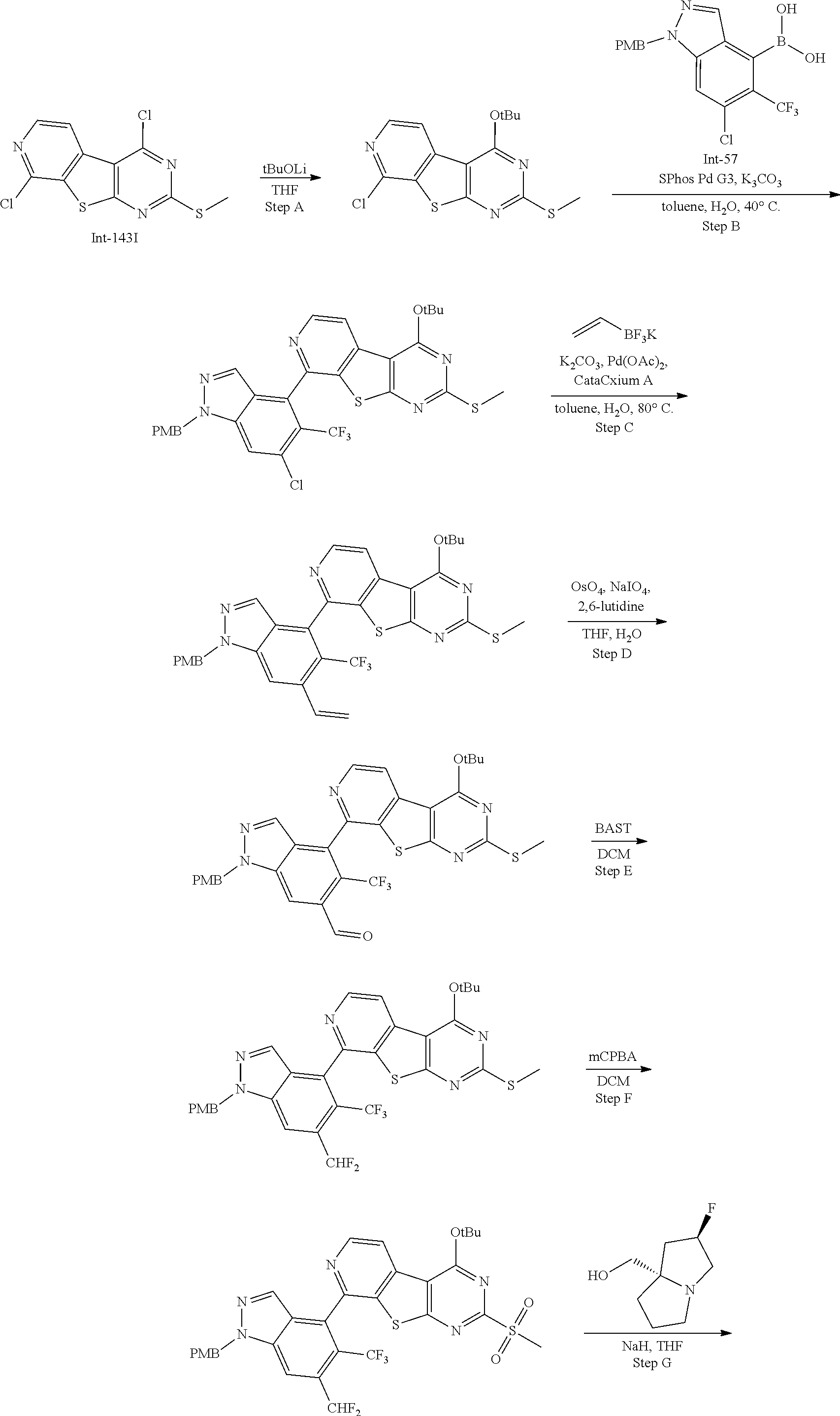

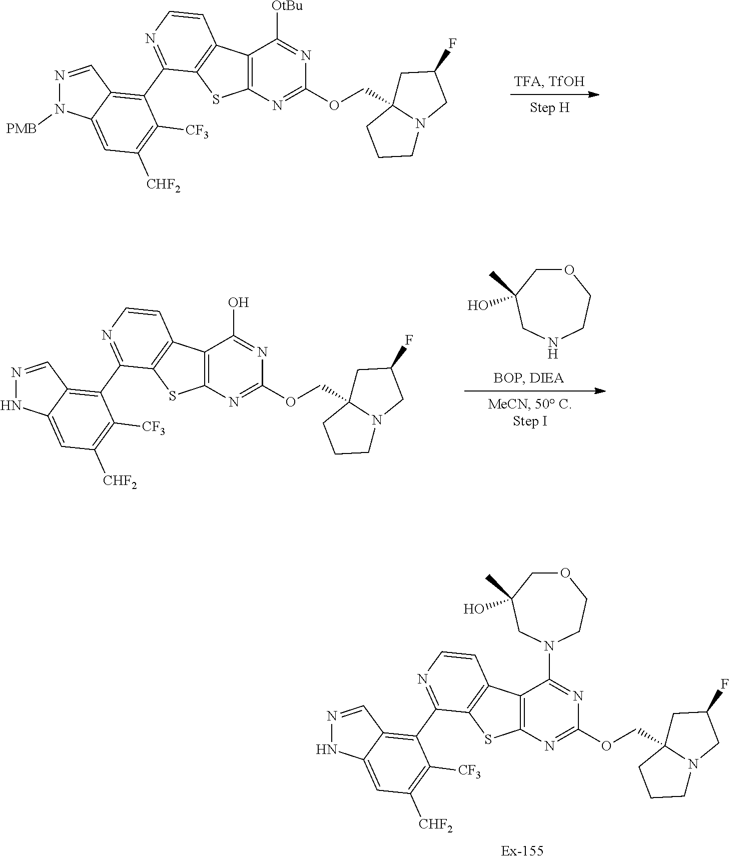

### Step A: 4-(tert-butoxy)-8-chloro-2-(methylthio)pyrido[4′,3′:4,5]thieno[2,3-d]pyrimidine

To a solution of 4,8-dichloro-2-(methylthio)pyrido[4′,3′:4,5]thieno[2,3-d]pyrimidine (Int-143I) (1 g, 3.31 mmol) in dry THF (10 mL) was added lithium tert-butoxide (4.96 mL, 4.96 mmol, 1 M in THF) dropwise at 0° C. under N₂. The solution was stirred at 20° C. for 20 h. Additional lithium tert-butoxide (0.159 g, 1.985 mmol) was added to the mixture at 0° C. The solution was stirred at 20° C. for 12 h. The mixture was diluted with water (30 mL) and extracted with EtOAc (3×50 mL). The organic layer was washed with brine (30 mL), dried over sodium sulfate, filtered, and the solvent was evaporated under reduced pressure to give the crude product. The crude product was purified by flash silica gel chromatography (0 to 15% EtOAc in petroleum ether) to give 4-(tert-butoxy)-8-chloro-2-(methylthio)pyrido[4′,3′:4,5]thieno[2,3-d]pyrimidine. MS (ESI) [M+H]⁺: m/z 340.

### Step B: 4-(tert-butoxy)-8-(6-chloro-1-(4-methoxybenzyl)-5-(trifluoromethyl)-1H-indazol-4-yl)-2-(methylthio)pyrido[4′,3′:4,5]thieno[2,3-d]pyrimidine

To a solution of 4-(tert-butoxy)-8-chloro-2-(methylthio)pyrido[4′,3′:4,5]thieno[2,3-d]pyrimidine (220 mg, 0.647 mmol) in toluene (3 mL) was added (6-chloro-1-(4-methoxybenzyl)-5-(trifluoromethyl)-1H-indazol-4-yl)boronic acid (Int-57) (242.6 mg, 0.631 mmol), K₂CO₃ (1.295 mL, 2.59 mmol, 2 M in H₂O) and (2-dicyclohexylphosphino-2′,6′-dimethoxybiphenyl) [2-(2′-amino-1,1′-biphenyl)]palladium (II) methanesulfonate (253 mg, 0.324 mmol) at 20° C. The mixture was stirred at 40° C. for 2 h. The mixture was quenched with water (2 mL), extracted with EtOAc (2×10 mL), and the organic layer dried over sodium sulfate. The organic layer was filtered and the solvent was evaporated under reduced pressure to give the crude product. The crude material was purified by preparative TLC (SiO₂, petroleum ether:EtOAc=3:1) to give 4-(tert-butoxy)-8-(6-chloro-1-(4-methoxybenzyl)-5-(trifluoromethyl)-1H-indazol-4-yl)-2-(methylthio)pyrido[4′,3′:4,5]thieno[2,3-d]pyrimidine. MS (ESI) [M+H]⁺: m/z 644.

### Step C: 4-(tert-butoxy)-8-(1-(4-methoxybenzyl)-5-(trifluoromethyl)-6-vinyl-1H-indazol-4-yl)-2-(methylthio)pyrido[4′,3′:4,5]thieno[2,3-d]pyrimidine

To solution of 4-(tert-butoxy)-8-(6-chloro-1-(4-methoxybenzyl)-5-(trifluoromethyl)-1H-indazol-4-yl)-2-(methylthio)pyrido[4′,3′:4,5]thieno[2,3-d]pyrimidine (268 mg, 0.416 mmol) in toluene/water (4 mL, 3:1) was added potassium trifluoro (vinyl) borate (84 mg, 0.624 mmol), palladium (II) acetate (4.67 mg, 0.021 mmol), K₂CO₃ (173 mg, 1.248 mmol) and di(1-adamantyl)-n-butylphosphine (14.92 mg, 0.042 mmol) under nitrogen atmosphere at 25° C. The mixture was stirred at 80° C. for 2 h. The mixture was diluted with water (10 mL) and extracted with EtOAc (3×30 mL). The combined organic layers were washed with brine (2×10 mL), dried over sodium sulfate, filtered, and the solvent was evaporated under reduced pressure. The residue was purified by flash silica gel chromatography (0 to 35% EtOAc in petroleum ether) to give 4-(tert-butoxy)-8-(1-(4-methoxybenzyl)-5-(trifluoromethyl)-6-vinyl-1H-indazol-4-yl)-2-(methylthio)pyrido[4′,3′:4,5]thieno[2,3-d]pyrimidine. MS (ESI) [M+H]⁺: m/z 636.

### Step D: 4-(4-(tert-butoxy)-2-(methylthio)pyrido[4′,3′:4,5]thieno[2,3-d]pyrimidin-8-yl)-1-(4-methoxybenzyl)-5-(trifluoromethyl)-1H-indazole-6-carbaldehyde

To a solution of 4-(tert-butoxy)-8-(1-(4-methoxybenzyl)-5-(trifluoromethyl)-6-vinyl-1H-indazol-4-yl)-2-(methylthio)pyrido[4′,3′:4,5]thieno[2,3-d]pyrimidine (270 mg, 0.425 mmol) in THF (1.5 mL) and water (1.5 mL) was added 2,6-lutidine (0.099 mL, 0.849 mmol), sodium periodate (363 mg, 1.699 mmol), and osmium tetroxide (21.59 mg, 0.085 mmol) at 25° C. The mixture was stirred at 50° C. for 2 h. The reaction was cooled to room temperature, quenched with sat. aq. Na₂SO₃ (2 mL), and the mixture was extracted with EtOAc (3×5 mL). The combined organic layers were washed with brine (2 mL), dried over anhydrous sodium sulfate, filtered, and concentrated in vacuo. The residue was purified by preparative TLC (SiO₂, petroleum ether:EtOAc=3:1) to give 4-(4-(tert-butoxy)-2-(methylthio)pyrido[4′,3′:4,5]thieno[2,3-d]pyrimidin-8-yl)-1-(4-methoxybenzyl)-5-(trifluoromethyl)-1H-indazole-6-carbaldehyde. MS (ESI) [M+H]⁺: m/z 638.

### Step E: 4-(tert-butoxy)-8-(6-(difluoromethyl)-5-(trifluoromethyl)-1H-indazol-4-yl)-2-(methylthio)pyrido[4′,3′:4,5]thieno[2,3-d]pyrimidine

To a solution of 4-(4-(tert-butoxy)-2-(methylthio)pyrido[4′,3′:4,5]thieno[2,3-d]pyrimidin-8-yl)-1-(4-methoxybenzyl)-5-(trifluoromethyl)-1H-indazole-6-carbaldehyde (60 mg, 0.094 mmol) in DCM (1 mL) was added bis(2-methoxyethyl)aminosulfur trifluoride (0.035 mL, 0.189 mmol) at 0° C. under N₂. The mixture was stirred at 15° C. for 1 h. The mixture was purified directly by preparative TLC (SiO₂, petroleum ether:EtOAc=3:1) to give 4-(tert-butoxy)-8-(6-(difluoromethyl)-5-(trifluoromethyl)-1H-indazol-4-yl)-2-(methylthio)pyrido[4′,3′:4,5]thieno[2,3-d]pyrimidine. MS (ESI) [M+H]⁺: m/z 660.

### Step F: 4-(tert-butoxy)-8-(6-(difluoromethyl)-1-(4-methoxybenzyl)-5-(trifluoromethyl)-1H-indazol-4-yl)-2-(methylsulfonyl)pyrido[4′,3′:4,5]thieno[2,3-d]pyrimidine

To a solution of 4-(tert-butoxy)-8-(6-(difluoromethyl)-5-(trifluoromethyl)-1H-indazol-4-yl)-2-(methylthio)pyrido[4′,3′:4,5]thieno[2,3-d]pyrimidine (31 mg, 0.047 mmol) in DCM (1 mL) was added mCPBA (21 mg, 0.103 mmol, 85 wt %) at 20° C. The mixture was stirred at 20° C. for 1 h. The mixture was quenched with sat. aq. NaHCO₃ (0.5 mL), sat. aq. Na₂SO₃ (0.5 mL), and extracted with DCM (6 mL). The combined organic layers was dried over sodium sulfate, filtered, and the solvent was evaporated under reduced pressure. The residue was purified by preparative TLC (SiO₂, petroleum ether:EtOAc=1:1) to give 4-(tert-butoxy)-8-(6-(difluoromethyl)-1-(4-methoxybenzyl)-5-(trifluoromethyl)-1H-indazol-4-yl)-2-(methylsulfonyl)pyrido[4′,3′:4,5]thieno[2,3-d]pyrimidine. MS (ESI) [M+H]⁺: m/z 692.

### Step G: 4-(tert-butoxy)-8-(6-(difluoromethyl)-1-(4-methoxybenzyl)-5-(trifluoromethyl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine

To a solution of 4-(tert-butoxy)-8-(6-(difluoromethyl)-1-(4-methoxybenzyl)-5-(trifluoromethyl)-1H-indazol-4-yl)-2-(methylsulfonyl)pyrido[4′,3′:4,5]thieno[2,3-d]pyrimidine (23 mg, 0.033 mmol) and ((2R,7aS)-2-fluorotetrahydro-1H-pyrrolizin-7a(5H)-yl) methanol (10.6 mg, 0.067 mmol) in THF (0.5 mL) was added NaH (6.7 mg, 0.166 mmol, 60% in mineral oil) at 0° C. under nitrogen atmosphere. The mixture was stirred at 20° C. for 30 min. The mixture was diluted with water (0.5 mL) and sat. aq. NH₄Cl (0.5 mL), extracted with EtOAc (3×2 mL). The combined organics were dried over sodium sulfate, filtered, and the solvent was evaporated under reduced pressure. The residue was purified by preparative TLC (SiO₂, DCM:MeOH=15:1) to give 4-(tert-butoxy)-8-(6-(difluoromethyl)-1-(4-methoxybenzyl)-5-(trifluoromethyl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine. MS (ESI) [M+H]⁺: m/z 771.

### Step H: 8-(6-(difluoromethyl)-5-(trifluoromethyl)-1H-indazol-4-yl)-2-(((2R,7aS)=2-fluorotetrahydro-1H-pyrrolizin-7a(5H)-yl)methoxy)pyrido[4′,3′:4,5]thieno[2,3-d]pyrimidin-4-ol

To a solution of 4-(tert-butoxy)-8-(6-(difluoromethyl)-1-(4-methoxybenzyl)-5-(trifluoromethyl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidine (20 mg, 0,026 mmol) in TFA/TfOH (0.3 mL, 10/1) was stirred at 50° C. for 0.5 h. The mixture was diluted with MeCN (0.5 mL) and triethylamine (0.04 mL). The mixture was purified by preparative TLC (SiO₂, DCM:MeOH=10:1) to give 8-(6-(difluoromethyl)-5-(trifluoromethyl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-ol. MS (ESI) [M+H]⁺: m/z 595.

### Step 1:(6S)-4-(8-(6-(difluoromethyl)-5-(trifluoromethyl)-1H-indazol-4-yl)-2-(2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-155)

To a solution of 8-(6-(difluoromethyl)-5-(trifluoromethyl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-ol (10 mg, 0.017 mmol) in MeCN (0.5 mL) was added BOP (37.2 mg, 0.084 mmol) at 25° C. The mixture was stirred at 25° C. for 30 min. N,N-diisopropylethylamine (32.6 mg, 0.252 mmol) and(S)-6-methyl-1,4-oxazepan-6-ol hydrochloride (14.10 mg, 0.084 mmol) was added to the reaction solution, then the mixture was stirred at 50° C. for 1 h. The mixture was cooled to room temperature and purified by preparative reverse-phase HPLC (MeCN/H₂O w/formic acid modifier) to give (6S)-4-(8-(6-(difluoromethyl)-5-(trifluoromethyl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-155). MS (ESI) [M+H]⁺: m/z 708. ¹H NMR (400 MHz, CD₃OD) δ 8.83 (d, J=5.8 Hz, 1H), 8.39 (s, 1H), 8.07 (d, J=5.8 Hz, 1H), 7.69-7.77 (m, 1H), 7.32 (t. J=53.6 Hz, 1H), 5.55 (d, J=52 Hz, 1H), 4.56-4.77 (m, 2H), 4.02-4.31 (m, 4H), 3.84-3.98 (m, 5H), 3.70-3.79 (m, 1H), 3.47-3.59 (m, 2H), 2.57-2.78 (m, 2H), 2.34-2.44 (m, 2H), 2.21 (d. J=7.6 Hz, 1H), 1.30-1.38 (m, 1H), 1.17-1.25 (m, 3H).

### Example 156: (6S)-4-(8-(5-(tert-butyl)-6-chloro-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-156)

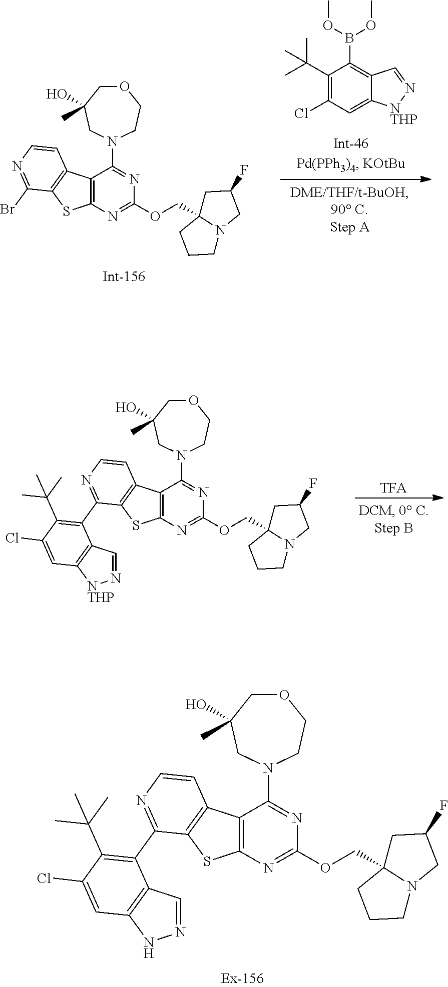

### Step A: (6S)-4-(8-(5-(tert-butyl)-6-chloro-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol

KOtBu (0.17 mL, 0.17 mmol, 1 M in THF) and Pd(PPh₃)₄ (20 mg, 0.017 mmol) were added to a solution of (5-(tert-butyl)-6-chloro-1-(tetrahydro-2H-pyran-2-yl)-1H-indazol-4-yl) boronate (Int-46) (32 mg, 0.088 mmol) and(S)-4-(8-bromo-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Int-156) (32 mg, 0.058 mmol) in DME (3.0 mL) and BuOH (0.17 mL). After stirring at 90° C. for 1 h, the solution was cooled to room temperature and diluted with EtOAc. After washing with brine, the organic layer was dried over Na₂SO₄, filtered, and evaporated. The crude residue was purified by silica gel chromatography (CHCl₃/EtOH) to afford (6S)-4-(8-(5-(tert-butyl)-6-chloro-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol. MS (ESI): m/z (M+H)⁺ 764.

### Step B: (6S)-4-(8-(5-(tert-butyl)-6-chloro-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-156)

TFA (0.40 mL) was added to a solution of (6S)-4-(8-(5-(tert-butyl)-6-chloro-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (8.5 mg, 0.011 mmol) in DCM (0.40 mL) at 0° C. After stirring at 0° C. for 3 h, the solution was diluted with CHCl₃ and quenched with sat. aq. NaHCO₃. The layers were separated and the aqueous phase was extracted with CHCl₃. The combined organic layers were washed with brine, dried over Na₂SO₄, filtered, and evaporated. The residue was purified by preparative reverse-phase HPLC (MeCN/H₂O w/formic acid modifier). Sat. aq. NaHCO₃ was added to the purified fractions containing the title compound, followed by extraction with ethyl acetate. The extract was washed with brine and dried over Na₂SO₄. The solvent was evaporated under reduced pressure to afford (6S)-4-(8-(5-(tert-butyl)-6-chloro-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Int-156). MS (ESI): m/z (M+H)⁺ 680. ¹H-NMR (400 MHz, CDCl₃) δ 8.75 (t, J=5.4 Hz, 1H), 7.69 (d, J=0.8 Hz, 1H), 7.59-7.55 (m, 1H), 7.10 (d, J=1.0 Hz, 1H), 6.89 (d, J=1.0 Hz, 1H), 5.26 (d, J=54.8 Hz, 1H), 4.31-4.05 (m, 5H), 4.00-3.96 (m, 1H), 3.83-3.60 (m, 4H), 3.48-3.14 (m, 5H), 2.96 (td, J=8.7, 3.5 Hz, 1H), 2.24-2.04 (m, 4H), 2.01-1.82 (m, 5H), 1.40 (s, 6H), 1.33 (s, 3H).

### Example 157: (6S)-4-(8-(1,5-dimethyl-6-(trifluoromethyl)-1H-indazol-7-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-157)

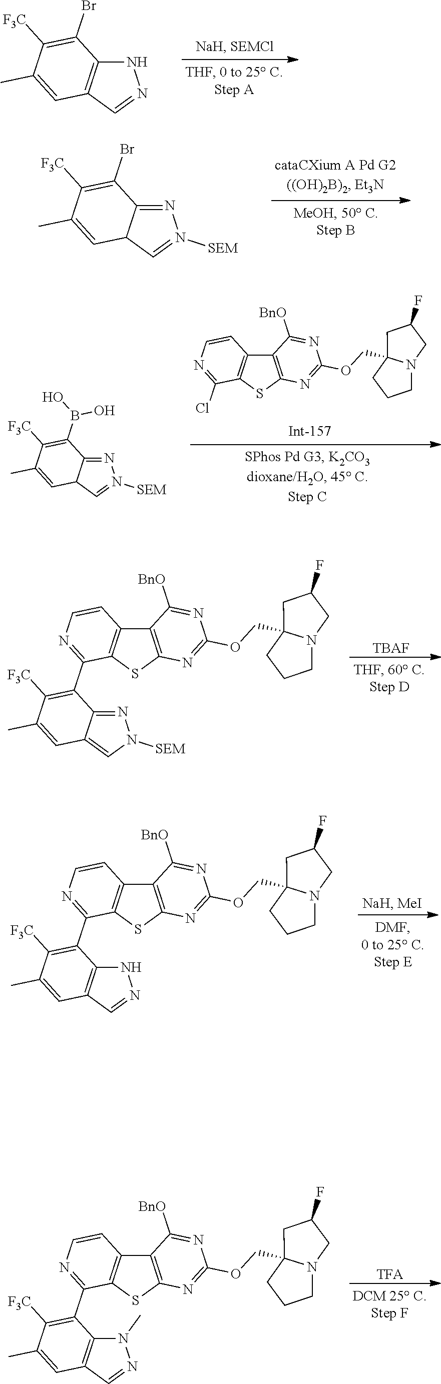

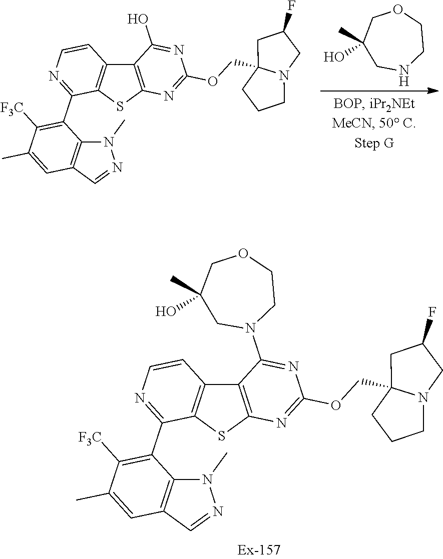

### Step A: 7-bromo-5-methyl-6-(trifluoromethyl)-2-((2-(trimethylsilyl)ethoxy)methyl)-2H-indazole

To a stirred solution of NaH (1.771 g, 44.3 mmol) and THF (70 ml) at 0° C. was added 7-bromo-5-methyl-6-(trifluoromethyl)-1H-indazole (7.2 g, 20.12 mmol) and the reaction mixture was stirred for 15 min. After 15 min, 2-(trimethylsilyl)ethoxymethyl chloride (5.71 mL, 32.2 mmol) was added and the reaction was stirred at 25° C. for 1 h. The reaction mixture was quenched with cold water (250 mL) at 0° C. and the reaction mixture was extracted with EtOAc (3×70 mL). The combined organic layers were dried over Na₂SO₄ and concentrated under reduced pressure. The crude product was purified via neutral alumina column chromatography (0 to 5% EtOAc in petroleum ether) to provide 7-bromo-5-methyl-6-(trifluoromethyl)-2-((2-(trimethylsilyl)ethoxy)methyl)-2H-indazole. MS (ESD): m/z (M+H)⁺ 409.

### Step B: (5-methyl-6-(trifluoromethyl)-2-((2-(trimethylsilyl) ethoxy)methyl)-2H-indazol-7-yl)boronic acid

A solution of 7-bromo-5-methyl-6-(trifluoromethyl)-2-((2-(trimethylsilyl)ethoxy)methyl)-2H-indazole (348 mg, 0.850 mmol), tetrahydroxydiboron (114 mg, 1.275 mmol), cataCXium A Pd G2 (56.8 mg, 0.085 mmol), and triethylamine (474 μL, 3.40 mmol) in MeOH (4.25 mL) was purged with N₂ and then allowed to stir at 50° C. for 1 h. The reaction mixture was cooled to room temperature and concentrated to give crude product. The crude residue was purified via silica gel chromatography (0 to 5% MeOH in DCM) to give (5-methyl-6-(trifluoromethyl)-2-((2-(trimethylsilyl)ethoxy)methyl)-2H-indazol-7-yl)boronic acid. MS (ESI): m/z (M+H)⁺ 374.

### Step C: 4-(benzyloxy)-2-(2R,7aS)-2-fluorotetrahydro-1H-pyrrolizin-7a(5H)-yl)methoxy)-8-(5-methyl-6-(trifluoromethyl)-2-((2-(trimethylsilyl)ethoxy)methyl)-2H-indazol-7-yl)pyrido[4′,3′:4,5]thieno[2,3-d]pyrimidine

A vial with stir bar was charged with (5-methyl-6-(trifluoromethyl)-2-((2-(trimethylsilyl)ethoxy)methyl)-2H-indazol-7-yl)boronic acid (386 mg, 1.031 mmol), 4-(benzyloxy)-8-chloro-2-(((2R,7aS)-2-fluorotetrahydro-1H-pyrrolizin-7a(5H)-yl)methoxy)pyrido[4′,3′:4,5]thieno[2,3-d]pyrimidine (Int-157) (400 mg, 0.825 mmol), and SPhos Pd G3 (515 mg, 0.660 mmol). The vial was fitted with a septum and purged with N₂ (3×). 1,4-dioxane (4.12 mL) and K₂CO₃ (4.12 mL, 8.25 mmol, 2 M in H₂O) were added, purged with nitrogen gas (×3), and the resultant mixture was allowed to stir at 45° C. for 1 h. The reaction mixture was cooled to room temperature and diluted with water. The aqueous phase was extracted with DCM and the combined organic layers were dried over MgSO₄, filtered, and concentrated to give 4-(benzyloxy)-2-(((2R,7aS)-2-fluorotetrahydro-1H-pyrrolizin-7a(5H)-yl)methoxy)-8-(5-methyl-6-(trifluoromethyl)-2-((2-(trimethylsilyl)ethoxy)methyl)-2H-indazol-7-yl)pyrido[4′,3′:4,5]thieno[2,3-d]pyrimidine. MS (ESI): m/z (M+H)⁺ 778.

### Step D: 4-(benzyloxy)-2-(((2R,7aS)-2-fluorotetrahydro-1H-pyrrolizin-7a(5H)-yl)methoxy)-8-(5-methyl-6-(trifluoromethyl)-1H-indazol-7-yl)pyrido[4′,3′:4,5]thieno[2,3-d]pyrimidine

To a solution of 4-(benzyloxy)-2-(((2R,7aS)-2-fluorotetrahydro-1H-pyrrolizin-7a(5H)-yl)methoxy)-8-(5-methyl-6-(trifluoromethyl)-2-((2-(trimethylsilyl)ethoxy)methyl)-2H-indazol-7-yl)pyrido[4′,3′:4,5]thieno[2,3-d]pyrimidine (208 mg, 0.267 mmol) in THF (2.67 mL) was added TBAF (401 μL, 0.401 mmol, 1 M in THF). The reaction mixture was allowed to stir at 60° C. for 18 h. The reaction mixture was cooled to room temperature and sat. aq. NaHCO₃ was added. The aqueous phase was extracted with EtOAc and the combined organic layers were dried over MgSO₄, filtered, and concentrated to give 4-(benzyloxy)-2-(((2R,7aS)-2-fluorotetrahydro-1H-pyrrolizin-7a(5H)-yl)methoxy)-8-(5-methyl-6-(trifluoromethyl)-1H-indazol-7-yl)pyrido[4′,3′:4,5]thieno[2,3-d]pyrimidine. MS (ESI): m/z (M+H)⁺ 648.

### Step E: 4-(benzyloxy)-8-(1,5-dimethyl-6-(trifluoromethyl)-1H-indazol-7-yl)-2-(((2R,7aS)-2-fluorotetrahydro-1H-pyrrolizin-7a(5H)-yl)methoxy)pyrido[4′,3′:4,5]thieno[2,3-d]pyrimidine

To solution of 4-(benzyloxy)-2-(((2R,7aS)-2-fluorotetrahydro-1H-pyrrolizin-7a(5H)-yl)methoxy)-8-(5-methyl-6-(trifluoromethyl)-1H-indazol-7-yl)pyrido[4′,3′:4,5]thieno[2,3-d]pyrimidine (173 mg, 0.267 mmol) in DMF (2.67 mL) at 0° C. was added sodium hydride (21.33 mg, 0.533 mmol, 60 wt % in mineral oil). The mixture was warmed to room temperature and stirred for 5 min. Iodomethane (41.7 μl, 0.667 mmol) was added and the resultant mixture was allowed to stir for 18 h. The reaction mixture was diluted with water and extracted with EtOAc. The combined organic layers were washed with 1% aq. LiCl, dried over magnesium sulfate, filtered, and concentrated to give crude product. The crude material was purified by preparative reverse-phase HPLC (MeCN/H₂O w/TFA modifier) to give 4-(benzyloxy)-8-(1,5-dimethyl-6-(trifluoromethyl)-1H-indazol-7-yl)-2-(((2R,7aS)-2-fluorotetrahydro-1H-pyrrolizin-7a(5H)-yl)methoxy)pyrido[4′,3′:4,5]thieno[2,3-d]pyrimidine. MS (ESI): m/z (M+H)⁺ 662.

### Step F: 8-(1,5-dimethyl-6-(trifluoromethyl)-1H-indazol-7-yl)-2-(((2R,7aS)-2-fluorotetrahydro-1H-pyrrolizin-7a(5H)-yl)methoxy)pyrido[4′,3′:4,5]thieno[2,3-d]pyrimidin-4-ol

To a solution of 4-(benzyloxy)-8-(1,5-dimethyl-6-(trifluoromethyl)-1H-indazol-7-yl)-2-(((2R,7aS)-2-fluorotetrahydro-1H-pyrrolizin-7a(5H)-yl)methoxy)pyrido[4′,3′:4,5]thieno[2,3-d]pyrimidine (5 mg, 7.54 μmol) in DCM (1 mL) was added TFA (1 mL) and the resultant mixture was allowed to stir at room temperature for 3 h. The reaction mixture was quenched with sat. aq. Na₂CO₃ and extracted with DCM. The combined organic layers were concentrated in vacuo to give 8-(1,5-dimethyl-6-(trifluoromethyl)-1H-indazol-7-yl)-2-(((2R,7aS)-2-fluorotetrahydro-1H-pyrrolizin-7a(5H)-yl)methoxy)pyrido[4′,3′:4,5]thieno[2,3-d]pyrimidin-4-ol. MS (ESI): m/z (M+H)⁺ 572.

### Step G: (6S)-4-(8-(1,5-dimethyl-6-(trifluoromethyl)-1H-indazol-7-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-157)

A solution of 8-(1,5-dimethyl-6-(trifluoromethyl)-1H-indazol-7-yl)-2-(((2R,7aS)-2-fluorotetrahydro-1H-pyrrolizin-7a(5H)-yl)methoxy)pyrido[4′,3′:4,5]thieno[2,3-d]pyrimidin-4-ol (5 mg, 8.73 μmol) and BOP (19.3 mg, 0.044 mmol) in acetonitrile (300 μL) was allowed to stir at room temperature for 1 h. To the reaction mixture was added (S)-6-methyl-1,4-oxazepan-6-ol (5.73 mg, 0.044 mmol) and N,N-diisopropylethylamine (22.9 μL, 0.131 mmol) and the resultant mixture was allowed to stir at 50° C. for 1 h. The reaction mixture was quenched with sat. aq. NaHCO₃, the aqueous phase was extracted with DCM, and the combined organic layers were concentrated to give crude product. The crude material was purified by preparative reverse-phase HPLC (MeCN/H₂O w/TFA modifier) to give (6S)-4-(8-(1,5-dimethyl-6-(trifluoromethyl)-1H-indazol-7-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-157) as the TFA salt. MS (ESI): m/z (M+H)⁺ 685.

### Example 158: (6S)-4-(8-(6-chloro-5-ethynyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-158)

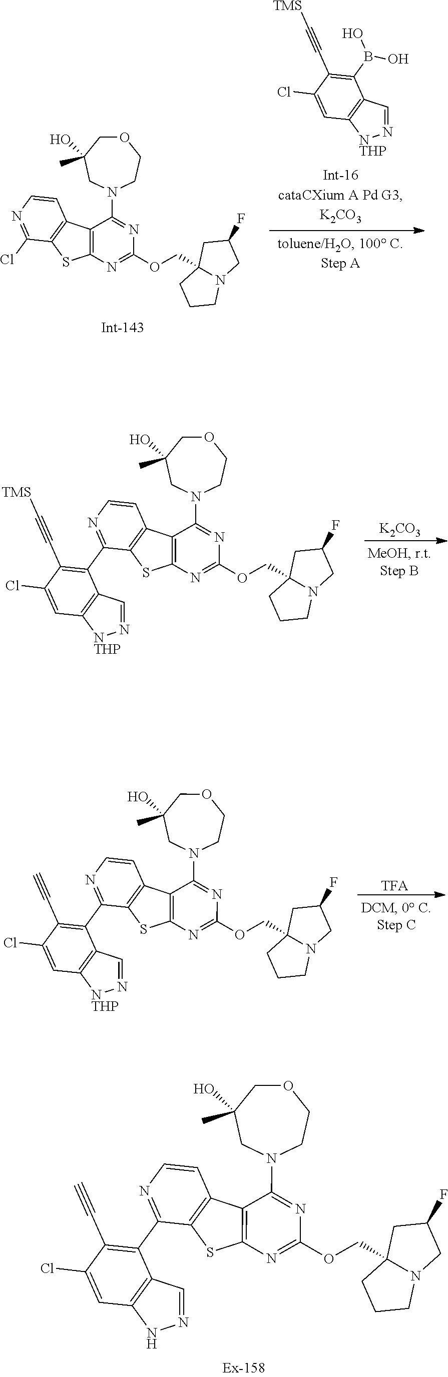

### Step A (6S)-4-(8-(6-chloro-1-(tetrahydro-2H-pyran-2-yl)-5-((trimethylsilyl) ethynyl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol

K₂CO₃ (0,094 mL, 0.19 mmol, 2 M in H₂O) and cataCXium A Pd G3 (11 mg, 0.015 mmol) were added to a solution of (6-chloro-1-(tetrahydro-2H-pyran-2-yl)-5-((trimethylsilyl) ethynyl)-1H-indazol-4-yl)boronic acid (Int-16) (29 mg, 0.076 mmol) and(S)-4-(8-chloro-2-(((2R,7aS)-2-fluorotetrahydro-1H-pyrrolizin-7a(5H)-yl)methoxy) pyrido[4′,3′:4,5]thieno[2,3-d]pyrimidin-4-yl)-6-methyl-1,4-oxazepan-6-ol (Int-143) (19 mg, 0.037 mmol) in toluene (1.0 mL). The reaction was warmed to 100° C. and stirred for 1 h. The solution was cooled to room temperature and diluted with EtOAc. After washing with brine, the organic layer was dried over Na₂SO₄, filtered, and evaporated. The residue was purified by silica gel chromatography (CHCl₃/EtOH) to afford (6S)-4-(8-(6-chloro-1-(tetrahydro-2H-pyran-2-yl)-5-((trimethylsilyl) ethynyl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol. MS (ESI): m/z (M+H)⁺ 804, 806.

### Step B: (6S)-4-(8-(6-chloro-5-ethynyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol

K₂CO₃ (2.3 mg, 0.016 mmol) was added to a solution of (6S)-4-(8-(6-chloro-1-(tetrahydro-2H-pyran-2-yl)-5-((trimethylsilyl) ethynyl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (6.6 mg, 0.0082 mmol) in MeOH (0.60 mL). After stirring at room temperature for 1 b, the solution was diluted with EtOAc and quenched with sat. aq. NH₄CL. The layers were separated and the aqueous phase was extracted with EtOAc. The combined organic layers were washed with brine, dried over Na₂SO₄, filtered, and evaporated to obtain (6S)-4-(8-(6-chloro-5-ethynyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol, which was used directly in the next step without further purification. MS (ESI): m/z (M+H)⁺ 732, 734.

### Step C: (6S)-4-(8-(6-chloro-5-ethynyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-158)

TFA (0.60 mL) was added to a solution of (6S)-4-(8-(6-chloro-5-ethynyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (5.8 mg, 0.0079 mmol) in DCM (0.60 mL) at 0° C. After stirring at 0° C. for 3 b, the solution was diluted with CHCl₃ and quenched with sat. aq. NaHCO₃. The layers were separated and the aqueous phase was extracted with CHCl₃. The combined organic layers were washed with brine, dried over Na₂SO₄, filtered, and evaporated. The residue was purified by preparative reverse-phase HPLC (MeCN/H₂O w/formic acid modifier). Sat. aq. NaHCO₃ was added to the purified fractions containing the title compound, followed by extraction with ethyl acetate. The extract was washed with brine and dried over Na₂SO₄. The solvent was evaporated under reduced pressure to afford (6S)-4-(8-(6-chloro-5-ethynyl-1H-indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-158). MS (ESI): m/z (M+H)⁺ 648, 650. ¹H NMR (400 MHz, CDCl₃) δ 8.90-8.81 (m, 1H), 7.76-7.64 (m, 3H), 5.38-5.23 (m, 1H), 4.32-4.09 (m, 5H), 3.96 (dd, J=12.5, 3.0 Hz, 1H), 3.83-3.62 (m, 4H), 3.45-3.32 (m, 3H), 3.21-3.16 (m, 2H), 3.05-2.92 (m, 1H), 2.33-2.18 (m, 4H), 2.08-1.95 (m, 2H), 1.42-1.37 (m, 3H).

### Example 159: (S)-4-(8-(4-ethynyl-1H-indazol-3-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-159)

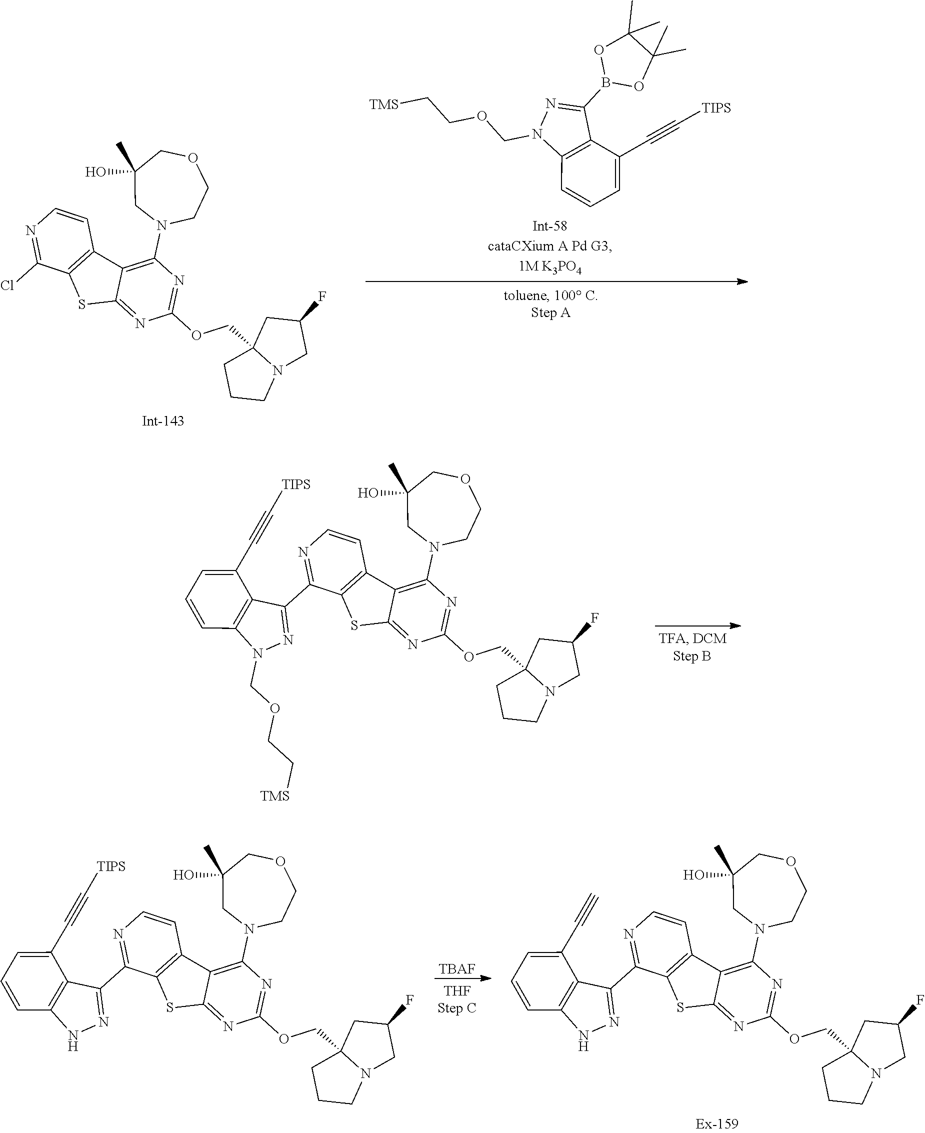

### Step A: (S)-4-(12-(2R,7aS)-2-fluorotetrahydro-1H-pyrrolizin-7a(5H)-yl)methoxy)-8-(4-((triisopropylsilyl)ethynyl)-1-((2-(trimethylsilyl)ethoxy)methyl)-1H-indazol-3-yl)pyrido[4′,3′:4,5]thieno[2,3-d]pyrimidin-4-yl)-6-methyl-1,4-oxazepan-6-ol

A mixture of(S)-4-(8-chloro-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Int-143) (30 mg, 0.059 mmol), 3-(4,4,5,5-tetramethyl-1,3,2-dioxaborolan-2-yl)-4-(triisopropylsilyl)ethynyl)-1-((2-(trimethylsilyl)ethoxy)methyl)-1H-indazole (Int-58) (66 mg, 0.118 mmol), cataCXium A Pd G3 (21 mg, 0.030 mmol) and K₃PO₄ (0.30 mL, 0.30 mmol, 1 M in H₂O) in toluene (1 mL) was degassed and nitrogen charged (3 times). Then the mixture was stirred at 110° C. under an atmosphere of nitrogen for 3 h. After cooling, the mixture was partitioned between EtOAc and water. The organic phase was washed with brine, dried over sodium sulfate, filtered, and concentrated. The crude residue was purified via silica gel chromatography (0 to 7% MeOH in chloroform) to give(S)-4-(2-(((2R,7aS)-2-fluorotetrahydro-1H-pyrrolizin-7a(5H)-yl)methoxy)-8-(4-((triisopropylsilyl)ethynyl)-1-((2-(trimethylsilyl)ethoxy)methyl)-1H-indazol-3-yl)pyrido[4′,3′:4,5]thieno[2,3-d]pyrimidin-4-yl)-6-methyl-1,4-oxazepan-6-ol which was used for next reaction without further purification. MS (ESI): m/z (M+H)⁺ 900.

### Step B: (S)-4-(2-(((2R,7aS)-2-fluorotetrahydro-1H-pyrrolizin-7a(5H)-yl)methoxy)-8-(4-((triisopropylsilyl)ethynyl)-1H-indazol-3-yl)pyrido[4′,3′:4,5]thieno[2,3-d]pyrimidin-4-yl)-6-methyl-1,4-oxazepan-6-ol

TFA (0.66 mL) was added to a stirred solution of(S)-4-(2-(((2R,7aS)-2-fluorotetrahydro-1H-pyrrolizin-7a(5H)-yl)methoxy)-8-(4-((triisopropylsilyl) ethynyl)-1-(2-(trimethylsilyl)ethoxy)methyl)-1H-indazol-3-yl)pyrido[4′,3′:4,5]thieno[2,3-d]pyrimidin-4-yl)-6-methyl-1,4-oxazepan-6-ol (30 mg, 0.033 mmol) in DCM (1.1 mL) at r.t. After being stirred for 1 h, the mixture was concentrated and the residue was partitioned between chloroform and sat. aq. NaHCO₃. The organic phase was dried over sodium sulfate, filtered, and concentrated. Methanol (1 mL) was added to the residue, along with water (0.12 mL) and potassium carbonate (25 mg, 0.18 mmol) and the resulting mixture was stirred for 10 min. The mixture was partitioned between EtOAc and water. The organic phase was washed with brine, dried over sodium sulfate, filtered, and concentrated to afford(S)-4-(2-(((2R,7aS)-2-fluorotetrahydro-1H-pyrrolizin-7a(5H)-yl)methoxy)-8-(4-((triisopropylsilyl)ethynyl)-1H-indazol-3-yl)pyrido[4′,3′:4,5]thieno[2,3-d]pyrimidin-4-yl)-6-methyl-1,4-oxazepan-6-ol which was used directly in the next step without further purification. MS (ESI): m/z (M+H)⁺ 770.

### Step C: (S)-4-(18-(4-ethynyl-1H-indazol-3-yl)-2-(2R,7aS)-2-fluorotetrahydro-1H-pyrrolizin-7a (5H)-yl)methoxy)pyrido[4′,3′:4,5]thieno[2,3-d]pyrimidin-4-yl)-6-methyl-1,4-oxazepan-6-ol (Ex-159)

TBAF (354 μL, 0.354 mmol) was added to a stirred solution of(S)-4-(2-(((2R,7aS)-2-fluorotetrahydro-1H-pyrrolizin-7a(5H)-yl)methoxy)-8-(4-((triisopropylsilyl)ethynyl)-1H-indazol-3-yl)pyrido[4′,3′:4,5]thieno[2,3-d]pyrimidin-4-yl)-6-methyl-1,4-oxazepan-6-ol in THF (1.2 mL) at r.t. An additional portion of TBAF (177 μL, 0.177 mmol) was added at 1.5 h then the mixture was stirred at 40° C. for another 1 h. The mixture was partitioned between EtOAc and water. The organic phase was washed with water and brine, dried over sodium sulfate, filtered and concentrated. The crude residue was purified via reverse phase HPLC (MeCN/H₂O with formic acid as modifier) to give(S)-4-(8-(4-ethynyl-1H-indazol-3-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-159). MS (ESI): m/z (M+H)⁺ 614. ¹H NMR (400 MHz, CDCl₃) δ 14.00-13.80 (brs, 1H), 8.50 (d, J=5.3 Hz, 1H), 7.62 (d, J=8.3 Hz, 1H), 7.52 (d, J=6.2 Hz, 1H), 7.43-7.39 (m, 1H), 7.00-6.99 (m, 1H), 6.56-6.52 (brs, 1H), 5.55-5.41 (m, 1H), 4.62-4.54 (m, 2H), 3.76-3.35 (m, 8H), 3.28 (s, 1H), 3.22-3.16 (m, 2H), 3.07-2.97 (m, 2H), 2.88-2.84 (m, 1H), 2.57-2.00 (m, 5H), 1.08 (s, 3H).

### Example 160: (6S)-4-(8-(5-ethynyl-6-fluoro-1H-benzo[f]indazol-4-yl)-2-(2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-160)

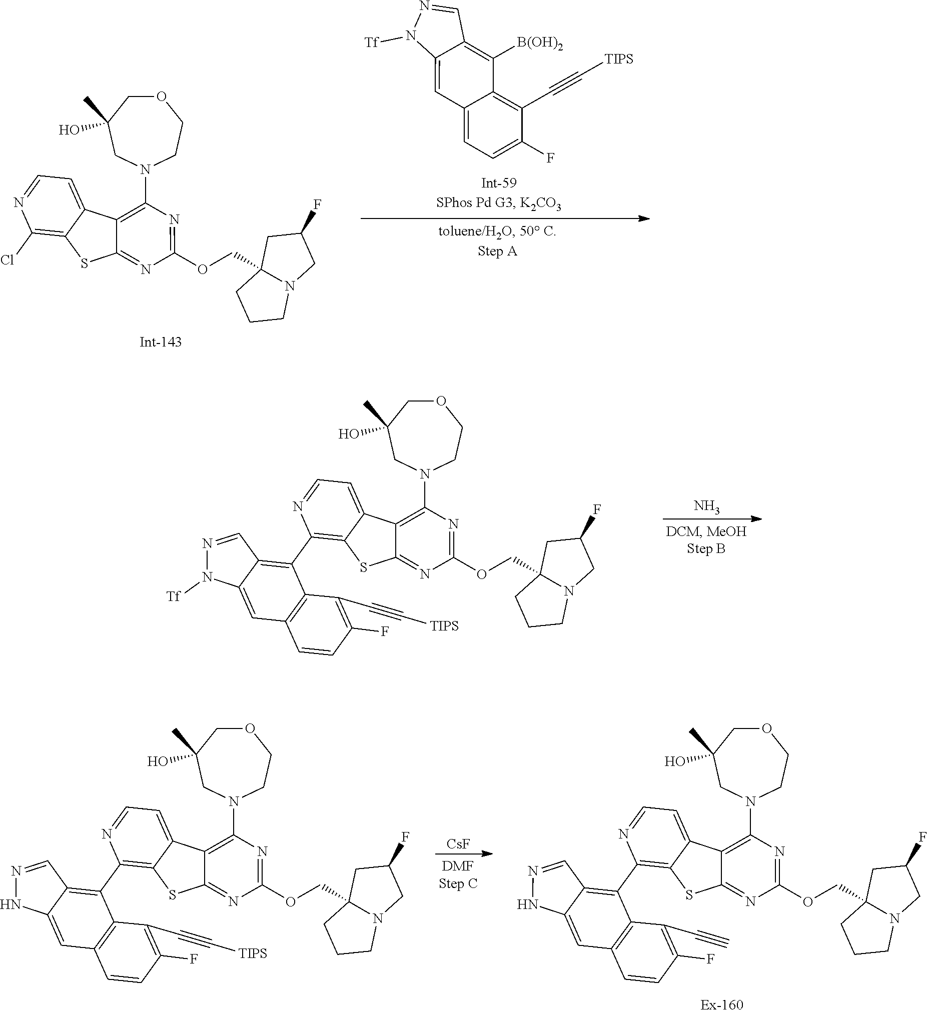

### Step A: (6S)-4-(8-(6-fluoro-1-((trifluoromethyl) sulfonyl)-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol

SPhos Pd G3 (307 mg, 0.394 mmol) and potassium carbonate (1.181 mL, 2.362 mmol, 2 M aqueous solution) were added to a solution of(S)-4-(8-chloro-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Int-143) (400 mg, 0.787 mmol) and (6-fluoro-1-((trifluoromethyl) sulfonyl)-5-((triisopropylsilyl)ethynyl)-1H-benzo[f]indazol-4-yl) boronic acid (Int-59) (427 mg, 0.787 mmol) in toluene (10 mL) at 25° C. The mixture was stirred at 50° C. for 3 h. The reaction was diluted with EtOAc (40 mL), washed with brine (3×8 mL) and the organic layer was concentrated in vacuo. The residue was purified by silica gel chromatography (petroleum ether/THF=1/2) to give (6S)-4-(8-(6-fluoro-1-((trifluoromethyl) sulfonyl)-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol. MS (ESI) [M+H]⁺: m/z 971.

### Step B: (6S)-4-(8-(6-fluoro-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol

Ammonia (8 mL, 56.0 mmol, 7 M in MeOH) was added to a solution of (6S)-4-(8-(6-fluoro-1-((trifluoromethyl) sulfonyl)-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (520 mg, 0.536 mmol) in DCM (1 mL) at 25° C., and the mixture was stirred for 16 h. The reaction mixture was concentrated in vacuo and the residue was purified by silica gel chromatography (DCM/MeOH=10/1) to give (6S)-4-(8-(6-fluoro-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol, MS (ESI) [M+H]⁺: m/z 839.

### Step C: (6S)-4-(8-(5-ethynyl-6-fluoro-1H-benzo[f]indazol-4-yl)-2-(2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepam-6-ol (Ex-160)

CsF (562 mg, 3.70 mmol) was added to a solution of (6S)-4-(8-(6-fluoro-5-((triisopropylsilyl)ethynyl)-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (310 mg, 0.370 mmol) in DMF (4.5 mL) at 25° C. and the mixture was stirred for 16 h. The reaction mixture was filtered and the filtrate was purified by preparative reverse-phase HPLC (MeCN/H₂O w/NH₄HCO₃ modifier) to give (6S)-4-(8-(5-ethynyl-6-fluoro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-260). MS (ESI) [M+H]⁺: m/z 682. ¹H NMR (400 MHz, CD₃OD) δ 8.76 (d, J=5.6 Hz, 1H), 8.38 (s, 1H), 8.25 (dd, J=5.8, 9.4 Hz, 1H), 7.99 (d, J=5.6 Hz, 1H), 7.82 (s, 1H), 7.43 (t, J=8.9 Hz, 1H), 5.40-5.17 (m, 1H), 4.30-4.09 (m, 4H), 4.05-3.86 (m, 3H), 3.77-3.68 (m, 2H), 3.56-3.42 (m, 1H), 3.22 (br d, J=7.7 Hz, 3H), 3.18-3.09 (m, 1H), 3.01 (dt, J=5.7, 9.5 Hz, 1H), 2.34-2.15 (m, 2H), 2.14-2.04 (m, 1H), 2.03-1.93 (m, 2H), 1.93-1.80 (m, 1H), 1.28-1.15 (m, 3H).

The examples in the table below were synthesized using a similar procedure as described in the synthesis of Ex-160 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | Variation | | |
|---|---|---|---|---|---|
| | | | in | | |
| | | | Reaction | | |
| Ex. | Starting Material | Structure | Conditions | Compound Name | [M + H]⁺ |
| Ex- 161 | 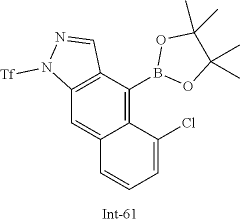 | 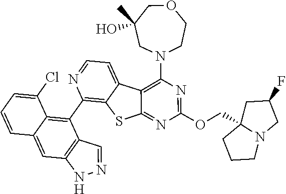 [SMILES: C[C@@]1(O)COCCN(c2nc(OC[C@@]34CCCN3C[C@H](F)C4)nc3sc4c(-c5c6cn[nH]c6cc6cccc(Cl)c56)nccc4c23)C1] | | (6S)-4-(8-(5-chloro- 1H-benzo[f]indazol- 4-yl)-2-(((2R,7aS)- 2-fluorotetrahydro- 1H-pyrrolizin- 7a(5H)-yl)methoxy)- pyrido[4′,3′:4,5] thieno[2,3-d]pyr- imidin-4-yl)-6-meth- yl-1,4-oxazepan-6-ol | 674 |
| Ex- 162 | 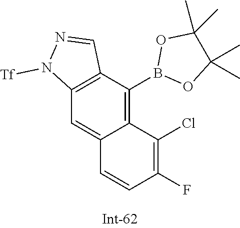 | 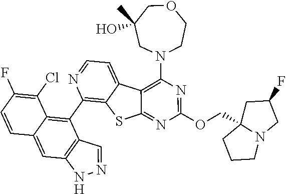 [SMILES: C[C@@]1(O)COCCN(c2nc(OC[C@@]34CCCN3C[C@H](F)C4)nc3sc4c(-c5c6cn[nH]c6cc6ccc(F)c(Cl)c56)nccc4c23)C1] | | (6S)-4-(8-(5-chloro- 6-fluoro-1H-benzo [f]indazol-4-yl)-2- (((2R,7aS)-2-fluoro- tetrahydro-1H-pyr- rolizin-7a(5H)-yl)- methoxy)pyrido [4′,3′:4,5]]thieno [2,3-d]pyrimidin- 4-yl)-6-methyl-1,4- oxazepan-6-ol | 692 |
| Ex- 163 | 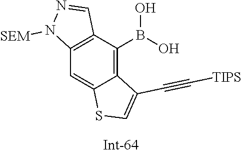 | 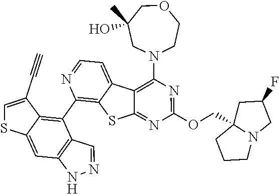 [SMILES: C#Cc1csc2cc3[nH]ncc3c(-c3nccc4c3sc3nc(OC[C@@]56CCCN5C[C@H](F)C6)nc(N5CCOC[C@@](C)(O)C5)c34)c12] | Step B: TFA, DCM, r.t. | (6S)-4-(8-(5-ethyn- yl-1H-thieno[3,2- f]indazol-4-yl)-2- (((2R,7aS)-2-fluoro- tetrahydro-1H-pyr- rolizin-7a(5H)-yl)- methoxy)pyrido [4′,3′:4,5]thieno[2,3- d]pyrimidin-4-yl)-6- methyl-1,4-oxaze- pan-6-ol | 670 |
| Ex- 164 | 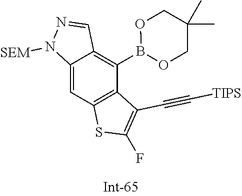 | 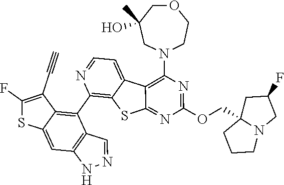 [SMILES: C#Cc1c(F)sc2cc3[nH]ncc3c(-c3nccc4c3sc3nc(OC[C@@]56CCCN5C[C@H](F)C6)nc(N5CCOC[C@@](C)(O)C5)c34)c12] | Step B: TFA, DCM, r.t. | (6S)-4-(8-(5-ethyn- yl-6-fluoro-1H- thieno[3,2-f] indazol-4-yl)-2- (((2R,7aS)-2-fluoro- tetrahydro-1H-pyr- rolizin-7a(5H)-yl)- methoxy)pyrido [4′,3′:4,5]thieno \|2,3-d]pyrimidin-4- yl)-6-methyl-1,4- oxazepan-6-ol | 688 |
| Ex- 165 | 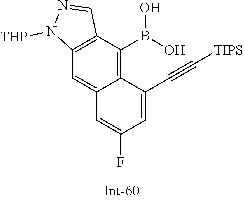 | 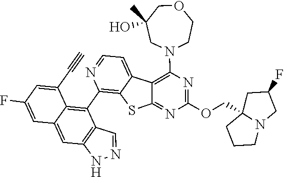 [SMILES: C#Cc1cc(F)cc2cc3[nH]ncc3c(-c3nccc4c3sc3nc(OC[C@@]56CCCN5C[C@H](F)C6)nc(N5CCOC[C@@](C)(O)C5)c34)c12] | Step B: TFA, DCM, r.t. | (6S)-4-(8-(5-ethyn- yl-7-fluoro-1H- benzo[f]indazol- 4-yl)-2-(((2R,7aS)- 2-fluorotetrahydro- 1H-pyrrolizin- 7a(5H)-yl)methoxy)- pyrido[4′,3′:4,5]thieno [2,3-d[pyrimidin-4- yl)-6-methyl-1,4- oxazepan-6-ol | 682 |
| Ex- 166 | 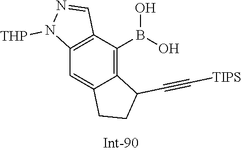 | 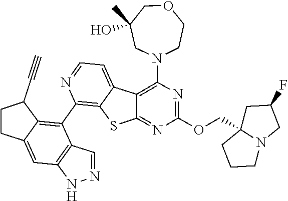 [SMILES: C#CC1CCc2cc3[nH]ncc3c(-c3nccc4c3sc3nc(OC[C@@]56CCCN5C[C@H](F)C6)nc(N5CCOC[C@@](C)(O)C5)c34)c21] | Step B: TFA, DCM, r.t. | (6S)-4-(8-(5-ethyn- yl-1,5,6,7-tetrahydro- cyclopenta[f]indazol- 4-yl)-2-(((2R,7aS)- 2-fluorotetrahydro- 1H-pyrrolizin- 7a(5H)-yl)methoxy)- pyrido[4′,3′:4,5] thieno[2,3-d]pyr- imidin-4-yl)-6-meth- yl-1,4-oxazepan-6-ol | 654 |

### Example 167: (6S)-4-(8-(5-ethyl-6-fluoro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-167)

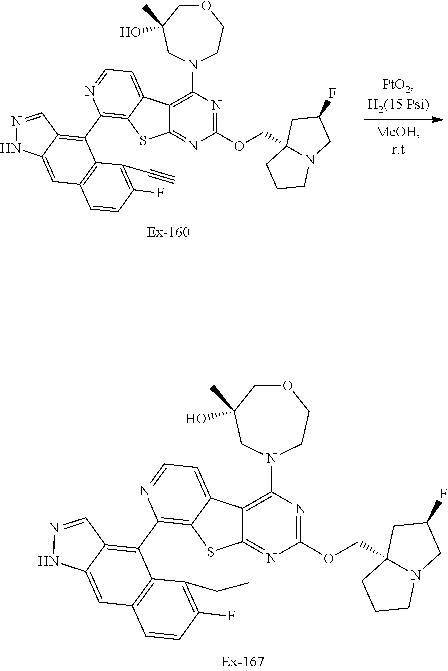 [SMILES: C#Cc1c(F)ccc2cc3[nH]ncc3c(-c3nccc4c3sc3nc(OC[C@@]56CCCN5C[C@H](F)C6)nc(N5CCOC[C@@](C)(O)C5)c34)c12 ; CCc1c(F)ccc2cc3[nH]ncc3c(-c3nccc4c3sc3nc(OC[C@@]56CCCN5C[C@H](F)C6)nc(N5CCOC[C@@](C)(O)C5)c34)c12]

Platinum (IV) oxide (150 mg, 0.661 mmol) was added to a solution of (6S)-4-(8-(5-ethynyl-6-fluoro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-160) (170 mg, 0.249 mmol) in MeOH (10 mL) at 25° C. under an atmosphere of N₂. The mixture was degassed and purged with hydrogen (3×) and stirred at 25° C. for 16 h under H₂ (15 psi). The reaction mixture was filtered and the filtrate was washed with MeOH (2×20 mL). The reaction was concentrated in vacuo and the residue was purified by preparative HPLC (MeCN/H₂O w/NH₄HCO₃ modifier), followed by preparative SFC (Column R, 30% EtOH w/0.1% NH₄OH) to give (6S)-4-(8-(5-ethyl-6-fluoro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-167) MS (ESI) [M+H]⁺: m/z 686. ¹H NMR (400 MHz, CD₃OD) δ 8.87-8.80 (m, 1H), 8.31 (s, 1H), 8.07 (dd, J=6.0, 9.2 Hz, 1H), 8.03-7.97 (m, 1H), 7.64-7.59 (m, 1H), 7.37 (t. J=9.3 Hz, 1H), 5.40-5.22 (m, 1H), 4.33-4.26 (m, 1H), 4.25-4.16 (m, 2H), 4.16-3.89 (m, 5H), 3.75-3.69 (m, 1H), 3.55-3.45 (m, 1H), 3.31-3.18 (m, 3H), 3.09-2.97 (m, 1H), 2.52-2.37 (m, 1H), 2.27-2.18 (m, 2H), 2.14-1.97 (m, 4H), 1.92-1.82 (m, 1H), 1.26-1.17 (m, 3H), 0.80-0.70 (m, 3H).

The examples in the table below were synthesized using a similar procedure as described in the synthesis of Ex-167 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | | [M + H]⁺ |
|---|---|---|---|---|
| Ex. | Starting Material | Structure | Compound Name | Found |
| Ex- 168 | 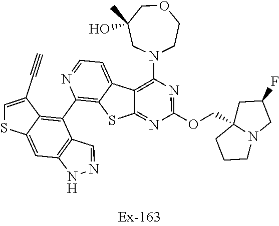 [SMILES: C#Cc1csc2cc3[nH]ncc3c(-c3nccc4c3sc3nc(OC[C@@]56CCCN5C[C@H](F)C6)nc(N5CCOC[C@@](C)(O)C5)c34)c12] | 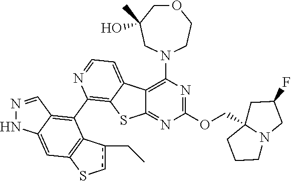 | (6S)-4-(8-(5-ethyl-1H- thieno[3,2-f]indazol- 4-yl)-2-(((2R,7aS)-2- fluorotetrahydro-1H- pyrrolizin-7a(5H)- yl)methoxy)pyrido [4′,3′:4,5]thieno[2,3- d]pyrimidin-4-yl)-6- methyl-1,4-oxazepan- 6-ol | 674 |
| Ex- 169 | 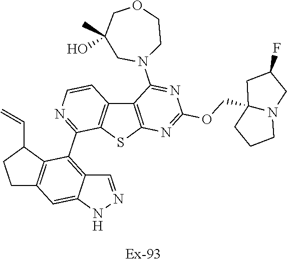 [SMILES: C=CC1CCc2cc3[nH]ncc3c(-c3nccc4c3sc3nc(OC[C@@]56CCCN5C[C@H](F)C6)nc(N5CCOC[C@@](C)(O)C5)c34)c21] | 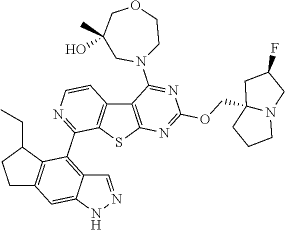 [SMILES: CCC1CCc2cc3[nH]ncc3c(-c3nccc4c3sc3nc(OC[C@@]56CCCN5C[C@H](F)C6)nc(N5CCOC[C@@](C)(O)C5)c34)c21] | (6S)-4-(8-(5-ethyl- 1,5,6,7-tetrahydro- cyclopenta[f]indazol- 4-yl)-2-(((2R,7aS)-2- fluorotetrahydro-1H- pyrrolizin-7a(5H)- yl)methoxy)pyrido [4′,3′:4,5]thieno[2,3- d]pyrimidin-4-yl)- 6-methyl-1,4-oxaze- pan-6-ol | 658 |

### Example 170: (6S)-4-(8-(1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-170) & Example 171: (6S)-4-(8-(5-ethynyl-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-171)

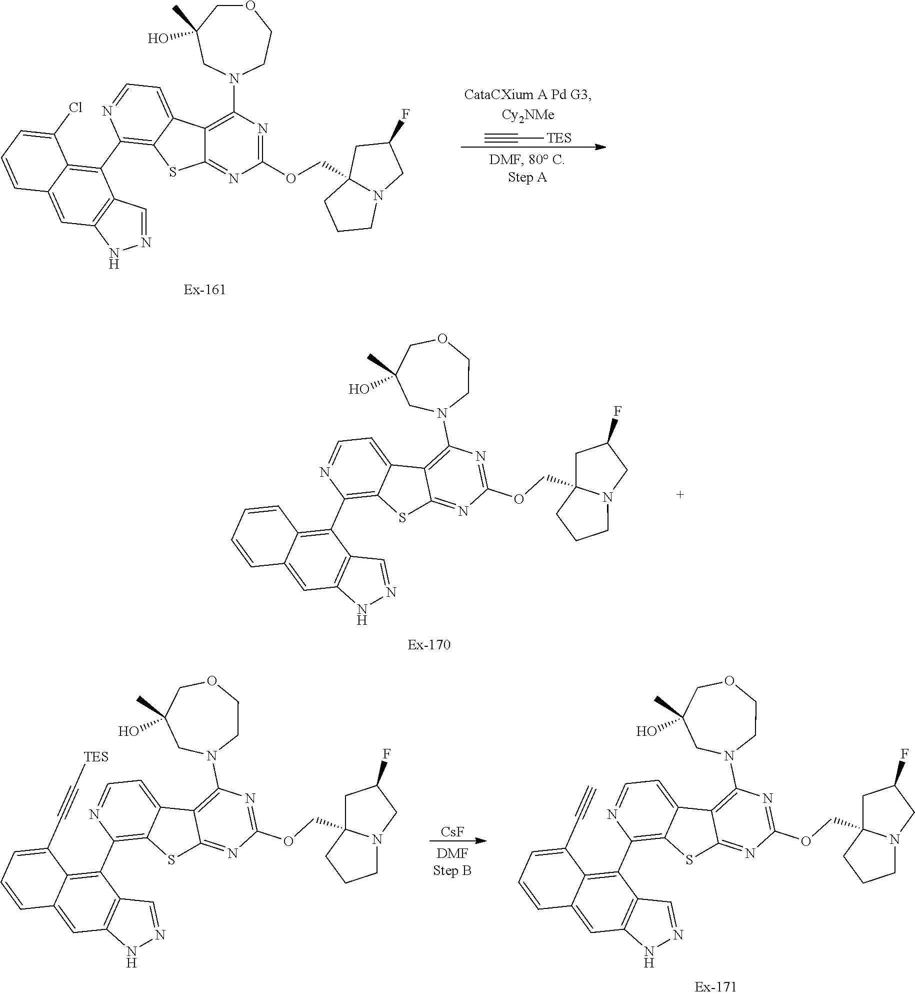

### Step A: (6S)-4-(2-(((2R,7aS)-2-fluorotetrahydro-1H-pyrrolizin-7a(5H)-yl)methoxy)-8-(5-((triethylsilyl) ethynyl)-1-benzo[f]indazol-4-yl)pyrido[4′,3′:4,5]thieno[2,3-d]pyrimidin-4-yl)-6-methyl-1,4-oxazepan-6-ol & (6S)-4-(8-(LH-benzo[f]indazol-4-yl)-2-(((2R,7aS)-2-fluorotetrahydro-1H-pyrrolizin-7a(5H)-yl)methoxy) pyrido[4′,3′:4,5]thieno[2,3-d]pyrimidin-4-yl)-6-methyl-1,4-oxazepan-6-ol (Ex-170)

A solution of (6S)-4-(8-(5-chloro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-161) (80 mg, 0.119 mmol) in DMF (3 mL) was added to a flask containing N,N-dicyclohexylmethylamine (185 mg, 0,949 mmol), triethyl(ethynyl) silane (166 mg, 1.187 mmol), and cataCXium A Pd G3 (43.3 mg, 0.059 mmol). The mixture was stirred at 80° C. for 16 h. The reaction was concentrated in vacuo. The residue was diluted with EtOAc (10 mL), washed with brine (2×1 mL), concentrated in vacuo, and the crude product was purified by preparative TLC (SiO₂, petroleum ether/THF (contained 1% NH₃ (7M in MeOH))=1/2) to give (6S)-4-(2-(((2R,7aS)-2-fluorotetrahydro-1H-pyrrolizin-7a(5H)-yl)methoxy)-8-(5-((triethylsilyl) ethynyl)-1H-benzo[f]indazol-4-yl)pyrido[4′,3′:4,5]thieno[2,3-d]pyrimidin-4-yl)-6-methyl-1,4-oxazepan-6-ol (MS (ESI) [M+H]⁺: m/z 778) and (6S)-4-(8-(1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-170). MS (ESI) [M+H]⁺: m/z 640. ¹H NMR (400 MHz, CD₃OD) δ 8.93 (d, J=5.7 Hz, 1H), 8.34 (s, 1H), 8.19-8.10 (m, 2H), 7.96 (d, J=9.2 Hz, 1H), 7.65 (t, J=8.8 Hz, 1H), 7.58-7.49 (m, 1H), 7.40-7.31 (m, 1H), 5.70-5.48 (m, 1H), 4.73-4.66 (m, 1H), 4.63-4.54 (m, 1H), 4.30 (br d, J=10.4 Hz, 1H), 4.19 (br d, J=15.0 Hz, 1H), 4.10-4.00 (m, 2H), 4.00-3.81 (m, 5H), 3.77 (d, J=13.7 Hz, 1H), 3.58-3.42 (m, 2H), 2.78-2.57 (m, 2H), 2.46-2.28 (m, 3H), 2.17 (br s, 1H), 1.23 (s, 3H).

### Step B: (6S)-4-(8-(5-ethynyl-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-171)

CsF (43.9 mg, 0.289 mmol) was added to a solution of (6S)-4-(2-(((2R,7aS)-2-fluorotetrahydro-1H-pyrrolizin-7a(5H)-yl)methoxy)-8-(5-((triethylsilyl) ethynyl)-1H-benzo[f]indazol-4-yl)pyrido[4′,3′:4,5]thieno[2,3-d]pyrimidin-4-yl)-6-methyl-1,4-oxazepan-6-ol (45 mg, 0.058 mmol) in DMF (1 mL) at 25° C. under an atmosphere of N₂ and the mixture was stirred for 16 h. The reaction mixture was filtered, and the filtrate was purified by preparative reverse-phase HPLC (MeCN/H₂O w/TFA modifier) to give (6S)-4-(8-(5-ethynyl-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-171) as the TFA salt. MS (ESI) [M+H]⁺: m/z 664. ¹H NMR (400 MHz, CD₃OD) δ 8.87 (dd, J=3.22, 6.08 Hz, 1H), 8.47-8.52 (m, 1H), 8.24-8.31 (m, 2H), 7.94-8.02 (m, 1H), 7.70-7.76 (m, 1H), 7.52-7.59 (m, 1H), 5.49-5.67 (m, 1H), 4.60-4.72 (m, 2H), 4.27 (br d, J=13.71 Hz, 1H), 4.19 (br d, J=14.54 Hz, 1H), 4.04-4.13 (m, 1H), 3.94-4.03 (m, 2H), 3.83-3.93 (m, 3H), 3.71-3.78 (m, 1H), 3.50-3.63 (m, 1H), 3.46 (td, J=5.42, 10.61 Hz, 1H), 3.07-3.21 (m, 1H), 2.76 (s, 1H), 2.54-2.73 (m, 2H), 2.29-2.45 (m, 3H), 2.03-2.14 (m, 1H), 1.24 (s, 3H).

The example in the table below was synthesized using a similar procedure as described in the synthesis of Ex-170 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | | [M + H]⁺ |
|---|---|---|---|---|
| Ex. | Starting Material | Structure | Compound Name | Found |
| Ex- 172 | 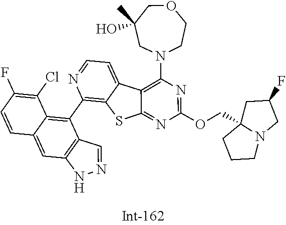 [SMILES: C[C@@]1(O)COCCN(c2nc(OC[C@@]34CCCN3C[C@H](F)C4)nc3sc4c(-c5c6cn[nH]c6cc6ccc(F)c(Cl)c56)nccc4c23)C1] | 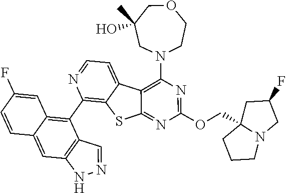 [SMILES: C[C@@]1(O)COCCN(c2nc(OC[C@@]34CCCN3C[C@H](F)C4)nc3sc4c(-c5c6cc(F)ccc6cc6[nH]ncc56)nccc4c23)C1] | (6S)-4-(8-(6-fluoro- 1H-benzo[f]indazol-4- yl)-2-(((2R,7aS)-2- fluorotetrahydro-1H- pyrrolizin-7a(5H)-yl)- methoxy)pyrido[4′,3′: 4,5]thieno[2,3-d]pyr- imidin-4-yl)-6-methyl- 1,4-oxazepam-6-ol | 658 |

### Example 173: (6S)-4-(8-(5-ethynyl-7,8-dihydro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-173)

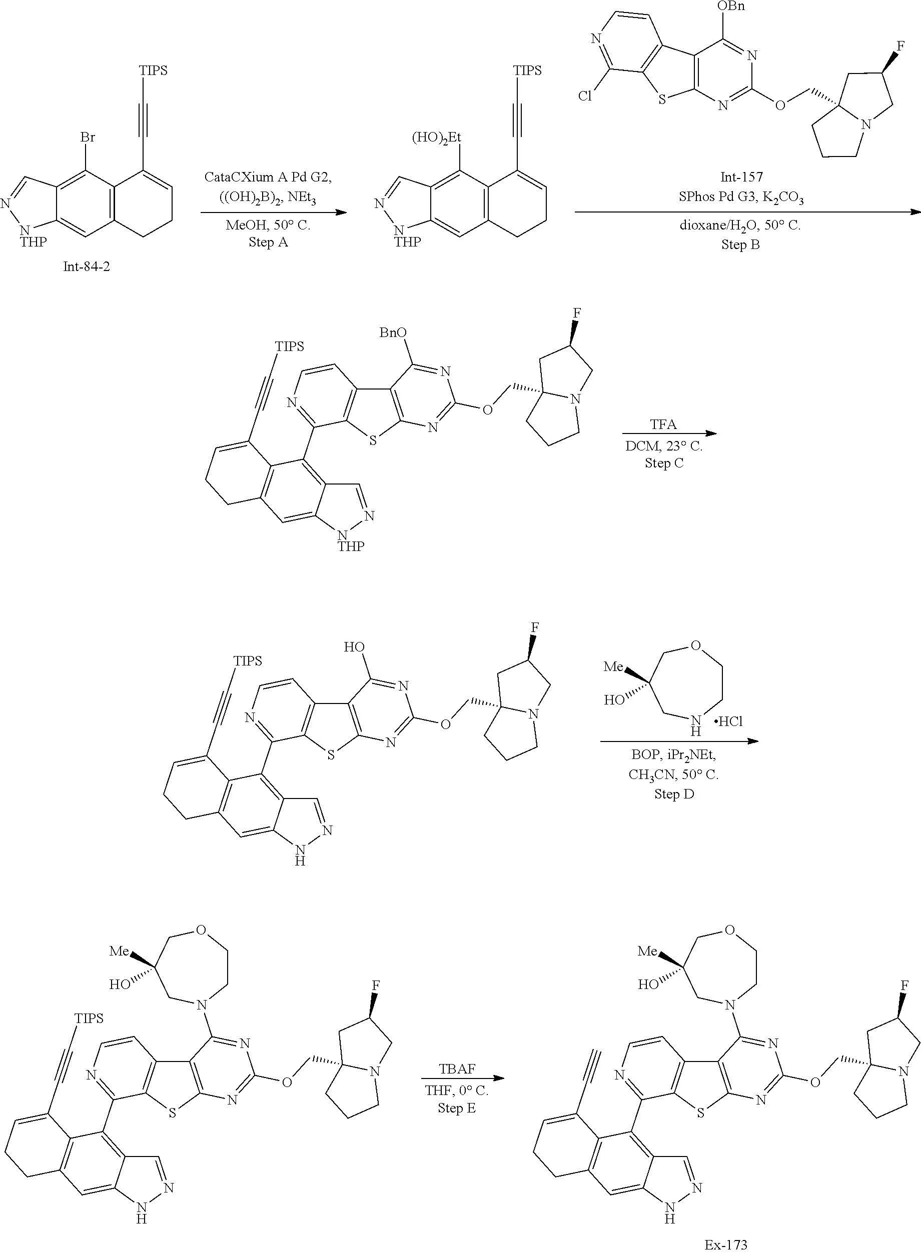

### Step A: (1-(tetrahydro-2H-pyran-2-yl)-5-((triisopropylsilyl)ethynyl)-7,8-dihydro-1-benzo[f]indazol-4-yl)boronic acid

A vial was charged with 4-bromo-1-(tetrahydro-2H-pyran-2-yl)-5-((triisopropylsilyl)ethynyl)-5,6,7,8-tetrahydro-1H-benzo[f]indazole (Int-84-2) (36 mg, 0.070 mmol), hypodiboric acid (19 mg, 0.210 mmol)), and cataCxium A Pd G2 (2.3 mg, 3.50 μmol) and dissolved in MeOH (1 mL) under an atmosphere of N₂. Triethylamine (15 μL, 0.108 mmol) was added via syringe. The mixture was stirred at 50° C. for 1 h. The mixture was concentrated in vacuo and redissolved in EtOAc (5 mL) and sat. aq. NH₄Cl (2 mL). The layers were shaken vigorously and separated. The aqueous phase was extracted with EtOAc (3×5 mL) and the combined organic layers were washed with brine, dried over Na₂SO₄, filtered, and concentrated under reduced pressure to obtain (1-(tetrahydro-2H-pyran-2-yl)-5-((triisopropylsilyl)ethynyl)-7,8-dihydro-1H-benzo[f]indazol-4-yl)boronic acid which was used without further purification. MS (ESI): [M+H]⁺ m/z 579.

### Step B: 4-(benzyloxy)-2-(((2R,7aS)-2-fluorotetrahydro-1H-pyrrolizin-7a(5H)-yl)methoxy)-8-(1-(tetrahydro-2H-pyran-2-yl)-5-((triisopropylsilyl) ethynyl)-7,8-dihydro-1H-benzo[f]indazol-4-yl)pyrido[4′,3′:4,5]thieno[2,3-d]pyrimidine

A vial was charged with (1-(tetrahydro-2H-pyran-2-yl)-5-((triisopropylsilyl)ethynyl)-7,8-dihydro-1H-benzo[f]indazol-4-yl)boronic acid (161 mg, 0.337 mmol), 4-(benzyloxy)-8-chloro-2-(((2R,7aS)-2-fluorotetrahydro-1H-pyrrolizin-7a(5H)-yl)methoxy)pyrido[4′,3′:4,5]thieno[2,3-d]pyrimidine (Int-157) (106 mg, 0.219 mmol)), and SPhos Pd G3 (87 mg, 0.111 mmol) and dioxane (3 mL). Under an atmosphere N₂, aqueous K₂CO₃ (0.843 mL, 1.685 mmol, 2 M in H₂O) was added via syringe. The mixture was stirred at 50° C. for 2 h. After this time, the mixture was diluted with water (2 mL), and extracted with DCM (3×5 mL), and the mixture was concentrated in vacuo. The crude mixture was purified via silica gel chromatography (10% MeOH in DCM) to obtain 4-(benzyloxy)-2-(((2R,7aS)-2-fluorotetrahydro-1H-pyrrolizin-7a(5H)-yl)methoxy)-8-(1-(tetrahydro-2H-pyran-2-yl)-5-((triisopropylsilyl)ethynyl)-7,8-dihydro-1H-benzo[f]indazol-4-yl)pyrido[4′,3′:4,5]thieno[2,3-d]pyrimidine. MS (ESI): [M+H]⁺ m/z 883.

### Step C: 2-(((2R,7aS)-2-fluorotetrahydro-1H-pyrrolizin-7a(5H)-yl)methoxy)-8-(5-((triisopropylsilyl)ethynyl)-7,8-dihydro-1H-benzo[f]indazol-4-yl)pyrido[4′,3′:4,5]thieno[2,3-d]pyrimidin-4-ol

A vial was charged with 4-(benzyloxy)-2-(((2R,7aS)-2-fluorotetrahydro-1H-pyrrolizin-7a(5H)-yl)methoxy)-8-(1-(tetrahydro-2H-pyran-2-yl)-5-((triisopropylsilyl)ethynyl)-7,8-dihydro-1H-benzo[f]indazol-4-yl)pyrido[4′,3′:4,5]thieno[2,3-d]pyrimidine (88 mg, 0.05 mmol) and TFA/DCM 1:1. The mixture was allowed to stir for 2.5 hours at room temperature. The volatiles were evaporated and the mixture was redissolved in CHCl₃/IPA (3:1, 5 mL) and aq. Na₂CO₃ (1 mL). The layers were separated and the aqueous layer was extracted with CHCl₃/IPA (3:1, 15 mL). The combined organic layers were concentrated under reduced pressure to provide 2-(((2R,7aS)-2-fluorotetrahydro-1H-pyrrolizin-7a(5H)-yl)methoxy)-8-(5-((triisopropylsilyl)ethynyl)-7,8-dihydro-1H-benzo[f]indazol-4-yl)pyrido[4′,3′:4,5]thieno[2,3-d]pyrimidin-4-ol. MS (ESI): [M+H]⁺ m/z 709.

### Step D: (6S)-4-(2-(((2R,7aS)-2-fluorotetrahydro-1H-pyrrolizin-7a(5H)-yl)methoxy)-8-(5-((triisopropylsilyl)ethynyl)-7,8-dihydro-1H-benzo[f]indazol-4-yl)pyrido[4′,3′:4,5]thieno[2,3-d]pyrimidin-4-yl)-6-methyl-1,4-oxazepan-6-ol

BOP (111 mg, 0.250 mmol) was added to a solution of 2-(((2R,7aS)-2-fluorotetrahydro-1H-pyrrolizin-7a(5H)-yl)methoxy)-8-(5-((triisopropylsilyl)ethynyl)-7,8-dihydro-1H-benzo[f]indazol-4-yl)pyrido[4′,3′:4,5]thieno[2,3-d]pyrimidin-4-ol (35 mg, 0.05 mmol) in MeCN (1 mL) at 25° C. The mixture was stirred at 25° C. for 30 min. N,N-diisopropylethylamine (0.174 mL, 1 mmol) and(S)-6-methyl-1,4-oxazepan-6-ol hydrochloride (42 mg, 0.250 mmol) were added to the reaction and the mixture was stirred at 50° C. for 2 h. The volatiles were removed to provide (6S)-4-(2-(((2R,7aS)-2-fluorotetrahydro-1H-pyrrolizin-7a(5H)-yl)methoxy)-8-(5-((triisopropylsilyl)ethynyl)-7,8-dihydro-1H-benzo[f]indazol-4-yl)pyrido[4′,3′:4,5]thieno[2,3-d]pyrimidin-4-yl)-6-methyl-1,4-oxazepan-6-ol. MS (ESI): m/z [M+H]⁺ 822.

### Step E: (6S)-4-(8-(5-ethynyl-7,8-dihydro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepam-6-ol (Ex-173)

TBAF (250 μL, 0.250 mmol, 1 M in THF) was added to a solution of (6S)-4-(2-(((2R,7aS)-2-fluorotetrahydro-1H-pyrrolizin-7a(5H)-yl)methoxy)-8-(5-((triisopropylsilyl)ethynyl)-7,8-dihydro-1H-benzo[f]indazol-4-yl)pyrido[4′,3′:4,5]thieno[2,3-d]pyrimidin-4-yl)-6-methyl-1,4-oxazepan-6-ol (41 mg, 0.05 mmol) in DMF (0.8 mL) via syringe and the mixture was heated at 80° C. for 3 h. The reaction was diluted with EtOAc (20 mL), washed with brine (3×3 mL), and the organic layer was concentrated in vacuo. The sample was purified via preparative reverse-phase HPLC (MeCN/H₂O w/NH₄OH) to provide (6S)-4-(8-(5-ethynyl-7,8-dihydro-1H-benzo[f]indazol-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-173). MS (ESI): m/z [M+H]⁺ 666. ¹H NMR (600 MHz, MeOD) δ 8.72-8.68 (m, 1H), 7.90-7.86 (m, 1H), 7.64 (s, 1H), 7.60 (s, 1H), 6.69-6.62 (m, 1H), 5.38-5.22 (m, 1H), 4.29 (dd, J=10.3, 3.1 Hz, 1H), 4.23-4.15 (m, 1H), 4.14-4.05 (m, 1H), 4.06-3.97 (m, 1H), 3.97-3.83 (m, 3H), 3.74-3.65 (m, 2H), 3.55-3.42 (m, 1H), 3.18 (s, 2H), 3.03 (dt, J=15.1, 6.5 Hz, 3H), 2.55 (s, 1H), 2.50-2.40 (m, 3H), 2.19 (dd, J=23.4, 14.9 Hz, 2H), 2.14-2.06 (m, 2H), 2.04-1.95 (m, 2H), 1.94-1.84 (m, 1H), 1.28-1.14 (m, 3H).

The examples in the table below were synthesized using a similar procedure as described in the synthesis of Ex-173 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | | [M + H]⁺ |
|---|---|---|---|---|
| Ex. | Starting Material | Structure | Compound Name | Found |
| Ex- 174 | 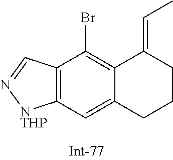 | 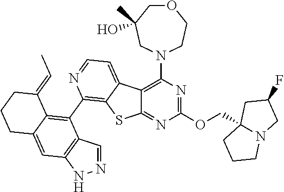 [SMILES: C/C=C1\CCCc2cc3[nH]ncc3c(-c3nccc4c3sc3nc(OC[C@@]56CCCN5C[C@H](F)C6)nc(N5CCOC[C@@](C)(O)C5)c34)c21] | (6S)-4-(8-((E)-5-ethyl- idene-5,6,7,8-tetrahydro- 1H-benzo[f]indazol-4- yl)-2-(((2R,7aS)-2-fluoro- tetrahydro-1H-pyrrolizin- 7a(5H)-yl)methoxy)pyr- ido[4′,3′:4,5]thieno[2,3- d]pyrimidin-4-yl)-6-meth- yl-1,4-oxazepan-6-ol | 670 |
| Ex- 175 | 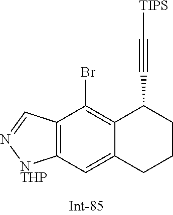 | 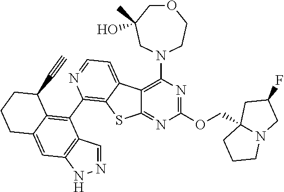 [SMILES: C#C[C@@H]1CCCc2cc3[nH]ncc3c(-c3nccc4c3sc3nc(OC[C@@]56CCCN5C[C@H](F)C6)nc(N5CCOC[C@@](C)(O)C5)c34)c21] | (6S)-4-(8-((S)-5-ethynyl- 5,6,7,8-tetrahydro-1H- benzo[flindazol-4-yl)-2- (((2R,7aS)-2-fluorotetra- hydro-1H-pyrrolizin- 7a(5H)-yl)methoxy)pyr- ido[4′,3′:4,5]thieno[2,3- d\|pyrimidin-4-yl)-6- methyl-1,4-oxazepan- 6-ol | 668 |
| Ex- 176 | 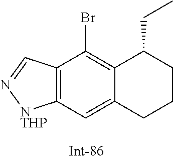 | 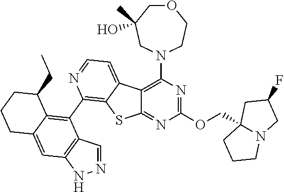 [SMILES: CC[C@@H]1CCCc2cc3[nH]ncc3c(-c3nccc4c3sc3nc(OC[C@@]56CCCN5C[C@H](F)C6)nc(N5CCOC[C@@](C)(O)C5)c34)c21] | (6S)-4-(8-((R)-5-ethyl- 5,6,7,8-tetrahydro-1H- benzo[/Jindazol-4-yl)-2- (((2R,7aS)-2-fluorotetra- hydro-1H-pyrrolizin- 7a(5H)-yl)methoxy)- pyrido[4′,3′:4,5]thieno [2,3-d]pyrimidin-4-yl)- 6-methyl-1,4-oxazepan- 6-ol | 672 |
| Ex- 177 | 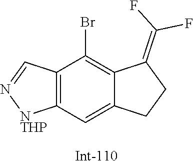 | 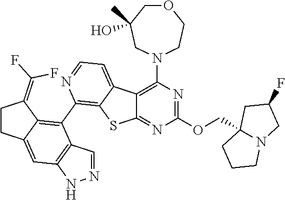 [SMILES: C[C@@]1(O)COCCN(c2nc(OC[C@@]34CCCN3C[C@H](F)C4)nc3sc4c(-c5c6c(cc7[nH]ncc57)CCC6=C(F)F)nccc4c23)C1] | (6S)-4-(8-(5-(difluoro- methylene)-1,5,6,7- tetrahydrocyclopenta [f]indazo1-4-yl)-2- (((2R,7aS)-2-fluoro- tetrahydro-1H-pyr- rolizin-7a(5H)-yl)- methoxy)pyrido[4′,3′: 4,5]thieno[2,3-d]pyr- imidin-4-yl)-6-methyl- 1,4-oxazepam-6-ol | 678 |

### Example 178: (S)-4-(8-(2-amino-5-ethynyl-6-fluoroquinolin-4-yl)-2-(((2R,7aS)-2-fluorotetrahydro-1H-pyrrolizin-7a(5H)-yl)methoxy)pyrido[4′,3′: 4.5]thieno[2,3-d]pyrimidin-4-yl)-6-methyl-1,4-oxazepan-6-ol (Ex-178)

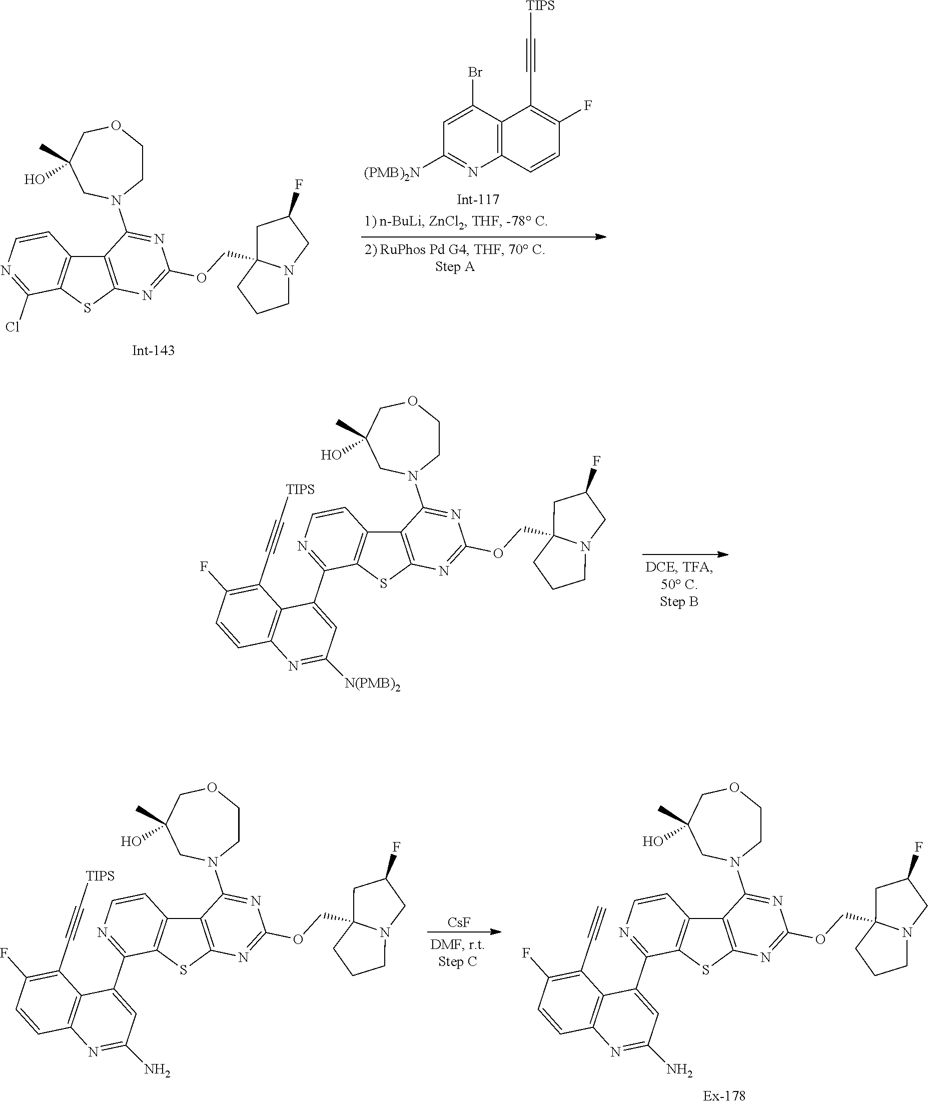

### Step(S)-4-(8-(2-(bis(4-methoxybenzyl)amino)-6-fluoro-5-((triisopropylsilyl)ethynyl) quinolin-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol

n-butyllithium (0.142 mL, 0.354 mmol, 2.5 M in hexanes) was added to a chilled solution (−78° C. of 4-bromo-6-fluoro-N,N-bis(4-methoxybenzyl)-5-((triisopropylsilyl)ethynyl) quinolin-2-amine (Int-117) (188 mg, 0.284 mmol) in THF (2 mL) and allowed to stir at −78° C. for 30 minutes. Zinc chloride (0.354 mL, 0.354 mmol, 1 M in THF) was added dropwise to the reaction mixture, and was then allowed to warm to room temperature and stir for 30 min. A nitrogen gas-purged solution of(S)-4-(8-chloro-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Int-143) (150 mg, 0.295 mmol) and RuPhos Pd G4 (25.1 mg, 0.030 mmol) in THF (2 mL) was added to the reaction mixture. The resulting reaction mixture was allowed to stir at 70° C. for 1 h. The reaction was cooled to r.t. and water was added. The mixture was extracted with EtOAc and the combined organics were dried over magnesium sulfate, filtered, and concentrated in vacuo. The crude residue was purified via silica gel chromatography (0 to 10% MeOH in DCM) to give(S)-4-(8-(2-(bis(4-methoxybenzyl)amino)-6-fluoro-5-((triisopropylsilyl)ethynyl) quinolin-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol. MS (ESI): m/z (M+H)⁺ 1054.

### Step B: (S)-4-(8-(2-amino-6-fluoro-5-((triisopropylsilyl)ethynyl) quinolin-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol

A solution of(S)-4-(8-(2-(bis(4-methoxybenzyl)amino)-6-fluoro-5-((triisopropylsilyl)ethynyl) quinolin-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (38 mg, 0.036 mmol) in TFA (0.5 mL, 6.49 mmol) and DCE (0.5 mL) was allowed to stir at 50° C. for 2 h. The reaction mixture was concentrated and sat. aq. NaHCO₃ was added. The aqueous phase was extracted with DCM, then the combined organics were dried over magnesium sulfate, filtered, and concentrated to give(S)-4-(8-(2-amino-6-fluoro-5-((triisopropylsilyl)ethynyl) quinolin-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol. MS (ESI): m/z (M+H)⁺ 814.

### Step C: (S)-4-(8-(2-amino-5-ethynyl-6-fluoroquinolin-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-178)

A solution of(S)-4-(8-(2-amino-6-fluoro-5-((triisopropylsilyl)ethynyl) quinolin-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (35 mg, 0.043 mmol) and cesium fluoride (13.1 mg, 0.086 mmol) in DMF (1 mL) was allowed to stir at room temperature for 2 h. Water was added and the mixture was extracted with EtOAc. The combined organic layers were dried over magnesium sulfate, filtered, and concentrated. The crude product was purified by preparative reverse phase HPLC (acetonitrile-water with NH₄OH as modifier) to give(S)-4-(8-(2-amino-5-ethynyl-6-fluoroquinolin-4-yl)-2-(((2R,7aS)-2-fluorotetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-178). MS (ESI): m/z (M+H)⁺ 658. ¹H NMR (500 MHz, DMSO-d⁶) δ 8.68 (s, 1H), 7.82 (s, 1H), 7.74-7.64 (m, 1H), 7.54 (t. J=9.0 Hz, 1H), 6.92 (s, 1H), 6.78 (s, 2H), 5.27 (t, J=27.2 Hz, 2H), 4.96 (s, 1H), 4.15-4.07 (m, 1H), 4.04-3.82 (m, 6H), 3.60-3.40 (m, 3H), 3.07 (s, 2H), 3.02 (s, 1H), 2.89-2.79 (m, 1H), 2.17-1.91 (m, 3H), 1.93-1.71 (m, 3H), 0.97 (d, J=5.4 Hz, 3H).

### Example 179:4-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidin-4-yl) morpholine (Ex-179)

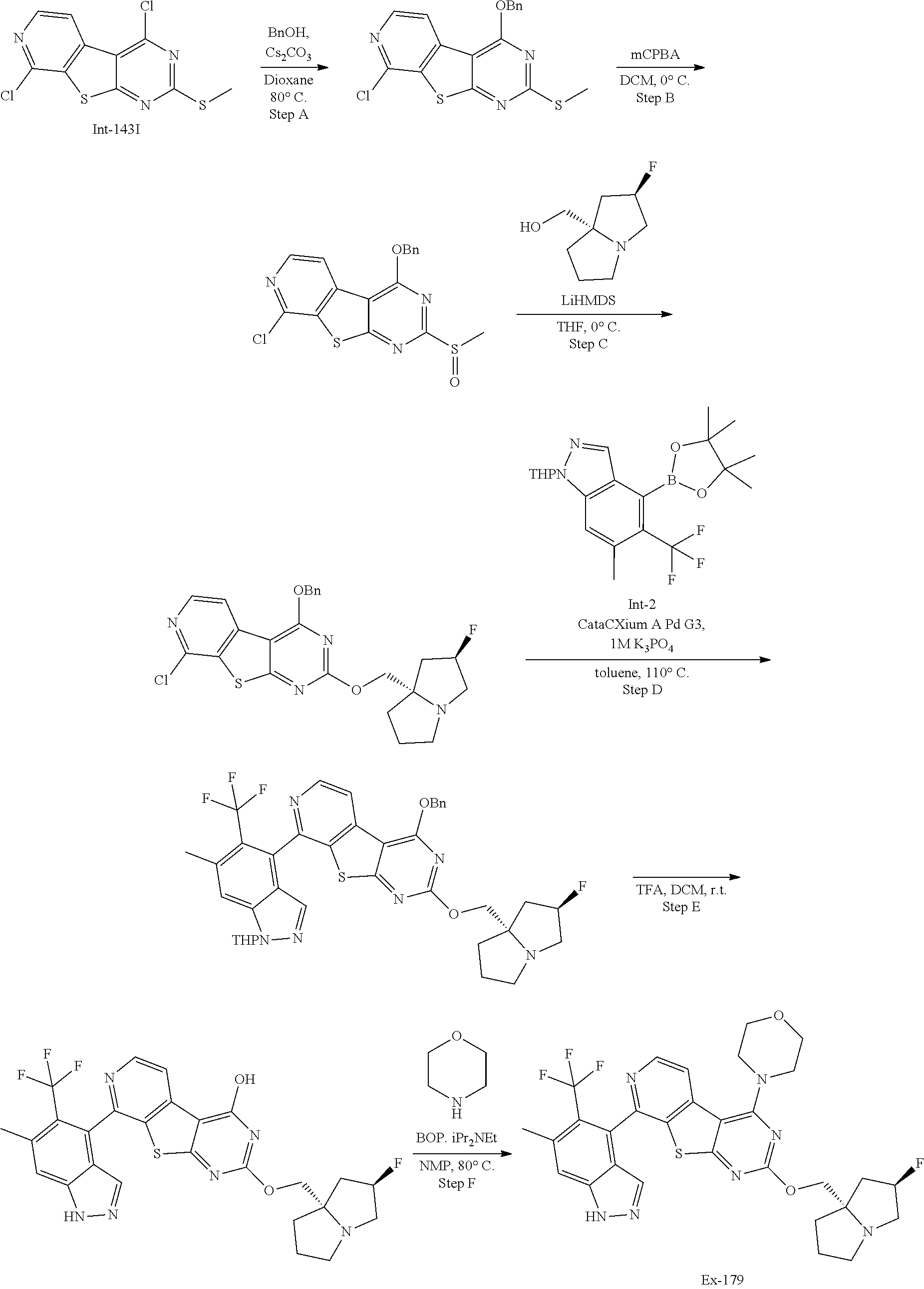

### Step A 4-(benzyloxy)-8-chloro-2-(methylthio)pyrido[4′,3′:4,5]thieno[2,3-d]pyrimidine

Benzyl alcohol (1.0 mL, 9.9 mmol) and Cs₂CO₃ (3.3 g, 10 mmol) were added to a solution of 4,8-dichloro-2-(methylthio)pyrido[4′,3′:4,5]thieno[2,3-d]pyrimidine (Int-143I) (1 g, 3.3 mmol) in 1,4-dioxane (17 mL). The mixture was heated to 80° C. and stirred for 5 h. After 5 h, the reaction was cooled to r.t. Water was added and the resulting solid was collected by filtration. The solid was heated to 50° C. under vacuum to dry, thereby obtaining 4-(benzyloxy)-8-chloro-2-(methylthio)pyrido[4′,3′:4,5]thieno[2,3-d]pyrimidine. MS (ESI): m/z (M+H)⁺ 374.

### Step B: 4-(benzyloxy)-8-chloro-2-(methylsulfinyl)pyrido[4′,3′:4,5]thieno[2,3-d]pyrimidine

mCPBA (610 mg, 2.6 mmol) was added to a solution of 4-(benzyloxy)-8-chloro-2-(methylthio)pyrido[4′,3′:4,5]thieno[2,3-d]pyrimidine (900 mg, 2.5 mmol) in DCM (60 mL) at 0° C. The mixture was stirred at 0° C. for 30 min. EtOAc, water, and sat. aq. NaHCO₃ were added and the layers were separated. The organic phase was washed with sat. aq. NaHCO₃ and brine, dried over sodium sulfate, filtered, and concentrated to give crude 4-(benzyloxy)-8-chloro-2-(methylsulfinyl)pyrido[4′,3′:4,5]thieno[2,3-d]pyrimidine. MS (ESI): m/z (M+H)⁺ 390.

### Step C: 4-(benzyloxy)-8-chloro-2-(((2R,7aS)-2-fluorotetrahydro-1H-pyrrolizin-7a(5H)-yl)methoxy)pyrido[4′,3′:4,5]thieno[2,3-d]pyrimidine

LiHMDS (4.8 mL, 4.8 mmol, 1M in THF) was added to a solution of ((2R,7aS)-2-fluorotetrahydro-1H-pyrrolizin-7a(5H)-yl) methanol (770 mg, 4.8 mmol) in THF (24 mL) at 0° C. The mixture was stirred at 0° C. for 20 min. The solution of crude 4-(benzyloxy)-8-chloro-2-(methylsulfinyl)pyrido[4′,3′:4,5]thieno[2,3-d]pyrimidine in THF (24 mL) was added dropwise at 0° C. and the reaction was stirred at 0° C. for 15 min. Water, EtOAc, and sat. ag. NaHCO₃ were added at 0° C. and the organic phase was washed with sat. aq. NaHCO₃ and brine, dried over sodium sulfate, filtered, and concentrated. The crude mixture was purified via silica gel chromatography (0 to 3% MeOH in chloroform) to give 4-(benzyloxy)-8-chloro-2-(((2R,7aS)-2-fluorotetrahydro-1H-pyrrolizin-7a(5H)-yl)methoxy)pyrido[4′,3′:4,5]thieno[2,3-d]pyrimidine. MS (ESI): m/z (M+H)⁺ 485.

### Step D: 4-(benzyloxy)-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidine

K₃PO₄ (2.3 mL, 2.3 mmol, 1 M in H₂O) was added to a mixture of 4-(benzyloxy)-8-chloro-2-(((2R,7aS)-2-fluorotetrahydro-1H-pyrrolizin-7a(5H)-yl)methoxy)pyrido[4′,3′:4,5]thieno[2,3-d]pyrimidine (220 mg, 0.45 mmol), 6-methyl-1-(tetrahydro-2H-pyran-2-yl)-4-(4,4,5,5-tetramethyl-1,3,2-dioxaborolan-2-yl)-5-(trifluoromethyl)-1H-indazole (Int-2) (320 mg, 0.77 mmol), and cataCXium A Pd G3 (170 mg, 0.23 mmol) in toluene (9.1 mL). The mixture was stirred at 110° C. for 6 hr. After 6 hr, the reaction was cooled to r.t. Water and EtOAc were added and the layers were separated. The organic phase was washed with brine, dried over sodium sulfate, filtered, and concentrated. The crude residue was purified by silica gel chromatography (0 to 4% MeOH in chloroform) to give 4-(benzyloxy)-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidine. MS (ESI): m/z (M+H)⁺ 733.

### Step E: 2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidin-4-ol

TFA (0.30 mL, 4.1 mmol) was added to a mixture of 4-(benzyloxy)-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidine (20 mg, 0.027 mmol) in DCM (0.55 mL). The mixture was stirred at r.t. for 2.5 hr. After 2.5 hr, the reaction was concentrated to give crude 2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidin-4-ol, which was used directly in the next step without further purification. MS (ESI): m/z (M+H)⁺ 559.

### Step F: 4-(2-(((2R,7aS)-2-fluorotetrahydro-1H-pyrrolizin-7a (5)-yl)methoxy)-8-(6-methyl-5-(trifluoromethyl)-1H-indazol-4-yl)pyrido[4′,3′:4,5]thieno[2,3-d]pyrimidin-4-yl) morpholine (Ex-60)

BOP (18 mg, 0.041 mmol) was added to a mixture of crude 2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidin-4-ol, iPr₂NEt (0.019 mL, 0.11 mmol), and morpholine (0.12 mL, 0.14 mmol) in NMP (0.55 mL) and the resulting mixture was stirred at 80° C. for 1.5 hr. The reaction was cooled to r.t. and EtOAc, water, and sat. aq. NaHCO₃ were added. The layers were separated and the organic phase was washed with water, sat. aq. NaHCO₃, and brine, dried over sodium sulfate, filtered, and concentrated. The residue was purified by preparative HPLC (MeCN/H₂O (0.1% formic acid)). Sat. aq. NaHCO₃ was added to the purified fractions containing the title compound, followed by extraction with ethyl acetate. The extract was washed with brine, dried over sodium sulfate, filtered, and evaporated under reduced pressure, thereby obtaining 4-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidin-4-yl) morpholine (Ex-179). MS (ESI): m/z (M+H)⁺ 628. ¹H NMR (400 MHz, CDCl₃) δ 10.95-10.04 (brs, 1H), 8.77 (d, J=5.49 Hz, 1H), 7.67 (d, J=5.50 Hz, 1H), 7.52 (s, 1H), 7.44 (s, 1H), 5.35-5.18 (m, 1H), 4.23 (d, J=10.29 Hz, 1H), 4.13 (d, J=10.76 Hz, 1H), 3.96-3.94 (m, 4H), 3.77-3.63 (m, 4H), 3.27-3.15 (m, 3H), 3.00-2.95 (m, 1H), 2.73-2.70 (brs, 3H), 2.32-2.09 (m, 3H), 1.98-1.82 (m, 3H).

The examples in the table below were synthesized using a similar procedure as described in the synthesis of Ex-179 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | | [M + H]⁺ |
|---|---|---|---|---|
| Ex. | Starting Material | Structure | Compound Name | Found |
| Ex- 180 | 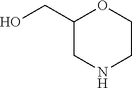 | 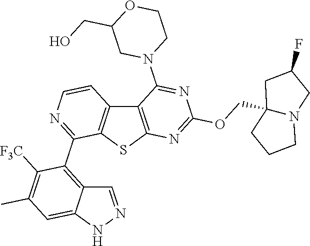 [SMILES: Cc1cc2[nH]ncc2c(-c2nccc3c2sc2nc(OC[C@@]45CCCN4C[C@H](F)C5)nc(N4CCOC(CO)C4)c23)c1C(F)(F)F] | (4-(2-(((2R,7aS)-2-fluoro- tetrahydro-1H-pyrrolizin- 7a(5H)-yl)methoxy)-8-(6- methyl-5-(trifluorometh- yl)-1H-indazol-4-yl)pyrido [4′,3′:4,5]thieno[2,3-d]pyr- imidin-4-yl)morpholin-2- yl)methanol | 658 |
| Ex- 181 | 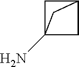 | 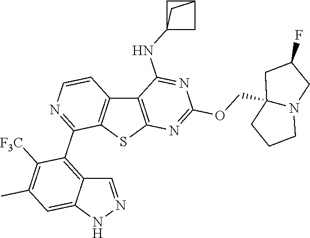 [SMILES: Cc1cc2[nH]ncc2c(-c2nccc3c2sc2nc(OC[C@@]45CCCN4C[C@H](F)C5)nc(NC45CC(C4)C5)c23)c1C(F)(F)F] | N-(bicyclo[1.1.1]pentan-1- yl)-2-(((2R,7aS)-2-fluoro- tetrahydro-1H-pyrrolizin- 7a(5H)-yl)-methoxy)-8-(6- methyl-5-(trifluoromethyl)- 1H-indazol-4-yl)pyrido [4′,3′:4,5]thieno[2,3-d] pyrimidin-4-amine | 624 |
| Ex- 182 | 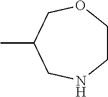 | 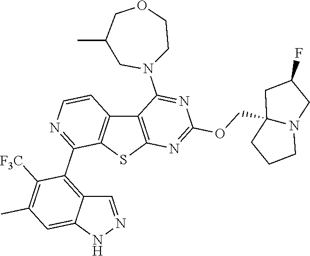 [SMILES: Cc1cc2[nH]ncc2c(-c2nccc3c2sc2nc(OC[C@@]45CCCN4C[C@H](F)C5)nc(N4CCOCC(C)C4)c23)c1C(F)(F)F] | 4-(2-(((2R,7aS)-2-fluoro- tetrahydro-1H-pyrrolizin- 7a(5H)-yl)methoxy)-8-(6- methyl-5-(trifluorometh- yl)-1H-indazol-4-yl)pyrido [4′,3′:4,5]thieno[2,3-d]pyr- imidin-4-yl)-6-methyl-1,4- oxazepane | 656 |
| Ex- 183 | 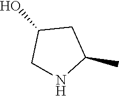 | 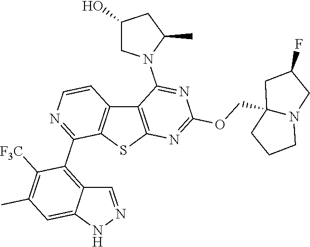 [SMILES: Cc1cc2[nH]ncc2c(-c2nccc3c2sc2nc(OC[C@@]45CCCN4C[C@H](F)C5)nc(N4C[C@H](O)C[C@H]4C)c23)c1C(F)(F)F] | (3R,5R)-1-(2-(((2R,7aS)- 2-fluorotetrahydro-1H- pyrrolizin-7a(5H)-yl) methoxy)-8-(6-methyl-5- (trifluoromethyl)-1H- indazol-4-yl)pyrido[4′,3′: 4,5]thieno[2,3-d]pyrimi- din-4-yl)-5-methylpyrrol- idin-3-ol | 642 |
| Ex- 184 | 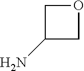 | 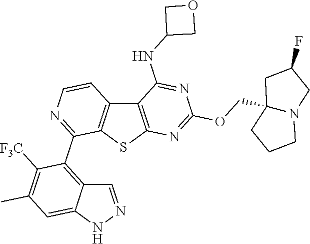 [SMILES: Cc1cc2[nH]ncc2c(-c2nccc3c2sc2nc(OC[C@@]45CCCN4C[C@H](F)C5)nc(NC4COC4)c23)c1C(F)(F)F] | 2-(((2R,7aS)-2-fluoro- tetrahydro-1H-pyrrolizin- 7a(5H)-yl)methoxy)-8-(6- methyl-5-(trifluorometh- yl)-1H-indazol-4-yl)-N- (oxetan-3-yl)pyrido [4′,3′:4,5]thieno[2,3-d] pyrimidin-4-amine | 614 |
| Ex- 185 | 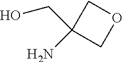 | 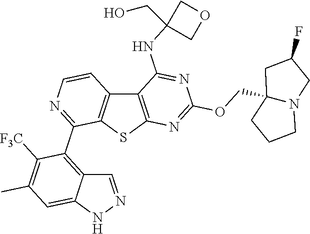 [SMILES: Cc1cc2[nH]ncc2c(-c2nccc3c2sc2nc(OC[C@@]45CCCN4C[C@H](F)C5)nc(NC4(CO)COC4)c23)c1C(F)(F)F] | (3-((2-(((2R,7aS)-2-fluoro- tetrahydro-1H-pyrrolizin- 7a(5H)-yl)methoxy)-8-(6- methyl-5-(trifluoromethyl)- 1H-indazol-4-yl)pyrido [4′,3′:4,5]thieno[2,3-d]pyr- imidin-4-yl)amino)oxetan- 3-yl)methanol | 644 |
| Ex- 186 | 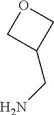 | 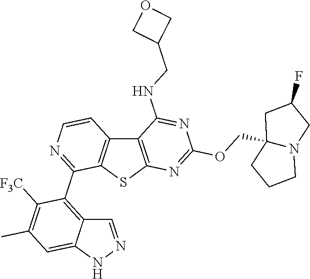 [SMILES: Cc1cc2[nH]ncc2c(-c2nccc3c2sc2nc(OC[C@@]45CCCN4C[C@H](F)C5)nc(NCC4COC4)c23)c1C(F)(F)F] | 2-(((2R,7aS)-2-fluorotetra- hydro-1H-pyrrolizin- 7a(5H)-yl)methoxy)-8-(6- methyl-5-(trifluorometh- yl)-1H-indazol-4-yl)-N- (oxetan-3-ylmethyl)pyrido [4′,3′:4,5]thieno[2,3-d] pyrimidin-4-amine | 628 |
| Ex- 187 | 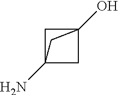 | 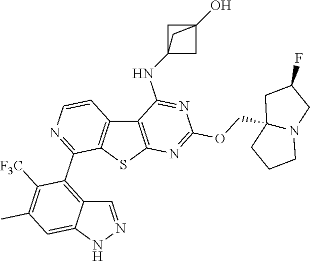 [SMILES: Cc1cc2[nH]ncc2c(-c2nccc3c2sc2nc(OC[C@@]45CCCN4C[C@H](F)C5)nc(NC45CC(O)(C4)C5)c23)c1C(F)(F)F] | 3-((2-(((2R,7aS)-2-fluoro- tetrahydro-1H-pyrrolizin- 7a(5H)-yl)methoxy)-8-(6- methyl-5-(trifluorometh- yl)-1H-indazol-4-yl)pyrido [4′,3′:4,5]thieno[2,3-d] pyrimidin-4-yl)amino)- bicyclo[1.1.1]pentan-1-ol | 640 |
| Ex- 188 | 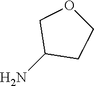 | 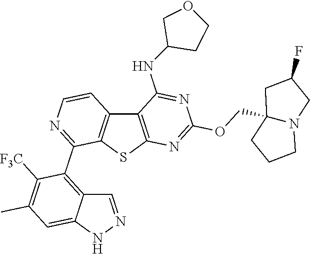 [SMILES: Cc1cc2[nH]ncc2c(-c2nccc3c2sc2nc(OC[C@@]45CCCN4C[C@H](F)C5)nc(NC4CCOC4)c23)c1C(F)(F)F] | 2-(((2R,7aS)-2-fluoro- tetrahydro-1H-pyrrolizin- 7a(5H)-yl)methoxy)-8-(6- methyl-5-(trifluoromethyl)- 1H-indazol-4-yl)-N-(tetra- hydrofuran-3-yl)pyrido [4′,3′:4,5 ]thieno[2,3-d] pyrimidin-4-amine | 628 |
| Ex- 189 | NH₃ | 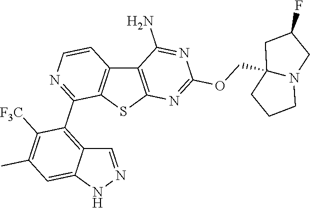 [SMILES: Cc1cc2[nH]ncc2c(-c2nccc3c2sc2nc(OC[C@@]45CCCN4C[C@H](F)C5)nc(N)c23)c1C(F)(F)F] | 2-(((2R,7aS)-2-fluoro- tetrahydro-1H-pyrrolizin- 7a(5H)-yl)methoxy)-8-(6- methyl-5-(trifluoromethyl)- 1H-indazol-4-yl)pyrido [4′,3′:4,5]thieno[2,3-d] pyrimidin-4-amine | 558 |
| Ex- 190 | 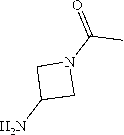 | 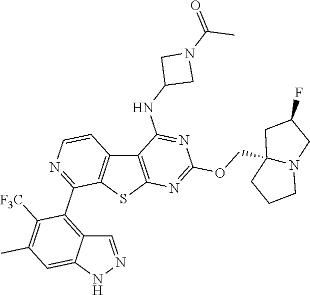 [SMILES: CC(=O)N1CC(Nc2nc(OC[C@@]34CCCN3C[C@H](F)C4)nc3sc4c(-c5c(C(F)(F)F)c(C)cc6[nH]ncc56)nccc4c23)C1] | 1-(3-((2-(((2R,7aS)-2- fluorotetrahydro-1H-pyr- rolizin-7a(5H)-yl)meth- oxy)-8-(6-methyl-5-(tri- fluoromethyl)-1H-indazol- 4-yl)pyrido[4′,3′:4,5] thieno[2,3-d]pyrimidin-4- yl)amino)azetidin-1-yl)- ethan-1-one | 655 |
| Ex- 191 | 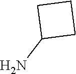 | 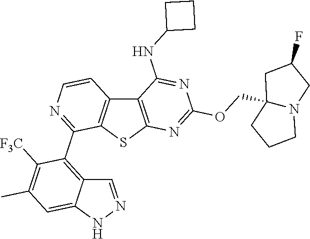 [SMILES: Cc1cc2[nH]ncc2c(-c2nccc3c2sc2nc(OC[C@@]45CCCN4C[C@H](F)C5)nc(NC4CCC4)c23)c1C(F)(F)F] | N-cyclobutyl-2-(((2R,7aS)- 2-fluorotetrahydro-1H- pyrrolizin-7a(5H)-yl) methoxy)-8-(6-methyl-5- (trifluoromethyl)-1H- indazol-4-yl)pyrido [4′,3′:4,5]thieno[2,3-d] pyrimidin-4-amine | 613 |
| Ex- 192 | 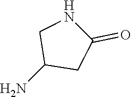 | 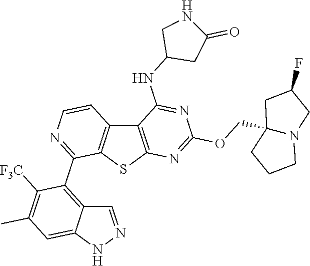 [SMILES: Cc1cc2[nH]ncc2c(-c2nccc3c2sc2nc(OC[C@@]45CCCN4C[C@H](F)C5)nc(NC4CNC(=O)C4)c23)c1C(F)(F)F] | 4-((2-(((2R,7aS)-2-fluoro- tetrahydro-1H-pyrrolizin- 7a(5H)-yl)methoxy)-8-(6- methyl-5-(trifluorometh- yl)-1H-indazol-4-yl)pyrido [4′,3′:4,5]thieno[2,3-d] pyrimidin-4-yl)amino)- pyrrolidin-2-one | 642 |
| Ex- 193 | 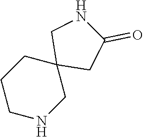 [SMILES: O=C1CC2(CCCNC2)CN1] | 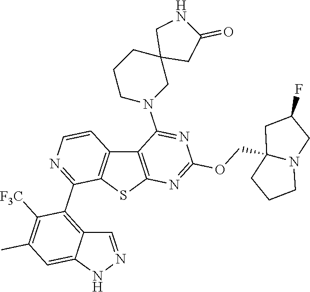 [SMILES: Cc1cc2[nH]ncc2c(-c2nccc3c2sc2nc(OC[C@@]45CCCN4C[C@H](F)C5)nc(N4CCCC5(CNC(=O)C5)C4)c23)c1C(F)(F)F] | 7-(2-(((2R,7aS)-2-fluoro- tetrahydro-1H-pyrrolizin- 7a(5H)-yl)methoxy)-8-(6- methyl-5-(trifluorometh- yl)-1H-indazol-4-yl)pyrido [4′,3′:4,5]thieno[2,3-d] pyrimidin-4-yl)-2,7-diaza- spiro[4.5]decan-3-one | 696 |
| Ex- 194 | 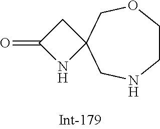 [SMILES: O=C1CC2(CNCCOC2)N1] | 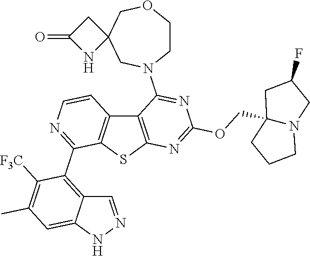 [SMILES: Cc1cc2[nH]ncc2c(-c2nccc3c2sc2nc(OC[C@@]45CCCN4C[C@H](F)C5)nc(N4CCOCC5(CC(=O)N5)C4)c23)c1C(F)(F)F] | 9-(2-(((2R,7aS)-2-fluoro- tetrahydro-1H-pyrrolizin- 7a(5/7)-yl)methoxy)-8-(6- methyl-5-(trifluorometh- yl)-1H-indazol-4-yl)pyrido [4′,3′:4,5]thieno[2,3-d] pyrimidin-4-yl)-6-oxa- 1,9-diazaspiro[3.6]decan- 2-one | 698 |
| Ex- 195 | 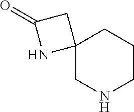 | 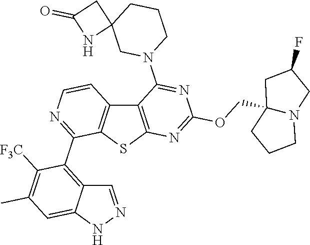 [SMILES: Cc1cc2[nH]ncc2c(-c2nccc3c2sc2nc(OC[C@@]45CCCN4C[C@H](F)C5)nc(N4CCCC5(CC(=O)N5)C4)c23)c1C(F)(F)F] | 6-(2-(((2R,7aS)-2-fluoro- tetrahydro-1H-pyrrolizin- 7a(5H)-yl)methoxy)-8-(6- methyl-5-(trifluorometh- yl)-1H-indazol-4-yl)pyrido [4′,3′:4,5]thieno[2,3-d] pyrimidin-4-yl)-1,6-diaza- spiro[3.5]nonan-2-one | 682 |
| Ex- 196 | 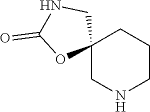 [SMILES: O=C1NC[C@@]2(CCCNC2)O1] | 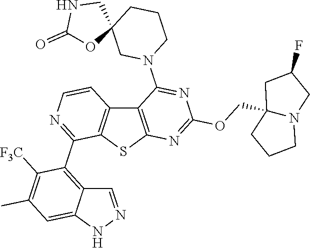 [SMILES: Cc1cc2[nH]ncc2c(-c2nccc3c2sc2nc(OC[C@@]45CCCN4C[C@H](F)C5)nc(N4CCC[C@@]5(CNC(=O)O5)C4)c23)c1C(F)(F)F] | (5R)-7-(2-(((2R,7aS)-2- fluorotetrahydro-1H-pyr- rolizin-7a(5)-yl)methoxy)- 8-(6-methyl-5-(trifluoro- methyl)-1H-indazol-4-yl)- pyrido[4′,3′:4,5]thieno[2,3- d]pyrimidin-4-yl)-1-oxa- 3,7-diazaspiro[4.5]decan- 2-one | 697 |
| Ex- 197 | 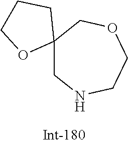 [SMILES: C1COC2(C1)CNCCOC2] | 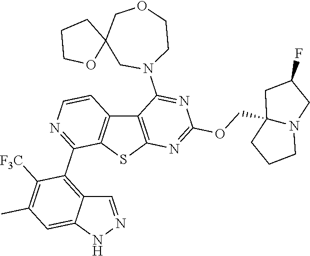 [SMILES: Cc1cc2[nH]ncc2c(-c2nccc3c2sc2nc(OC[C@@]45CCCN4C[C@H](F)C5)nc(N4CCOCC5(CCCO5)C4)c23)c1C(F)(F)F] | 10-(2-(((2R,7aS)-2-fluoro- tetrahydro-1H-pyrrolizin- 7a(5H)-yl)methoxy)-8-(6- methyl-5-(trifluorometh- yl)-1H-indazol-4-yl)pyrido [4′,3′:4,5]thieno[2,3-d] pyrimidin-4-yl)-1,7-dioxa- 10-azaspiro[4.6]undecane | 698 |
| Ex- 198 | 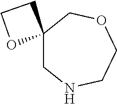 | 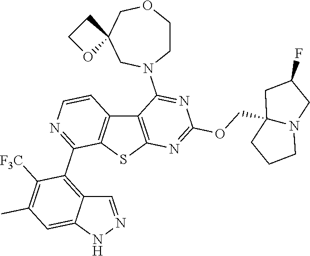 [SMILES: Cc1cc2[nH]ncc2c(-c2nccc3c2sc2nc(OC[C@@]45CCCN4C[C@H](F)C5)nc(N4CCOC[C@]5(CCO5)C4)c23)c1C(F)(F)F] | (4S)-9-(2-(((2R,7aS)-2- fluorotetrahydro-1H-pyr- rolizin-7a(5H)-yl)meth- oxy)-8-(6-methyl-5-(tri- fluoromethyl)-1H-indazol- 4-yl)pyrido[4′,3′:4,5] thieno[2,3-d]pyrimidin-4- yl)-1,6-dioxa-9-azaspiro [3.6]decane | 684 |
| Ex- 199 | 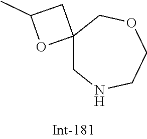 | 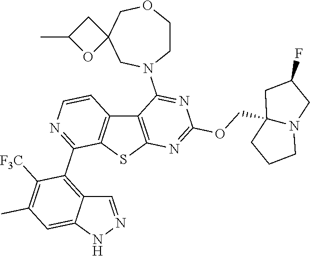 [SMILES: Cc1cc2[nH]ncc2c(-c2nccc3c2sc2nc(OC[C@@]45CCCN4C[C@H](F)C5)nc(N4CCOCC5(CC(C)O5)C4)c23)c1C(F)(F)F] | 9-(2-(((2R,7aS)-2-fluoro- tetrahydro-1H-pyrrolizin- 7a(5/7)-yl)methoxy)-8-(6- methyl-5-(trifluorometh- yl)-1H-indazol-4-yl)pyrido [4′,3′:4,5]thieno[2,3-d]pyr- imidin-4-yl)-2-methyl- 1,6-dioxa-9-azaspiro[3.6] decane | 698 |
| Ex- 200 | 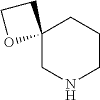 | 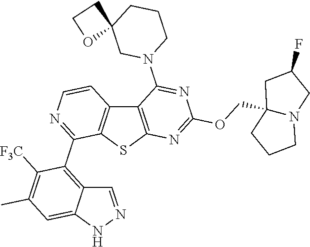 [SMILES: Cc1cc2[nH]ncc2c(-c2nccc3c2sc2nc(OC[C@@]45CCCN4C[C@H](F)C5)nc(N4CCC[C@]5(CCO5)C4)c23)c1C(F)(F)F] | (4S)-6-(2-(((2R,7aS)-2- fluorotetrahydro-1H-pyr- rolizin-7a(5H)-yl)meth- oxy)-8-(6-methyl-5-(tri- fluoromethyl])-1H-indazol- 4-yl)pyrido[4′,3′:4,5] thieno[2,3-d]pyrimidin-4- yl)-1-oxa-6-azaspiro[3.5] nonane | 668 |
| Ex- 201 | 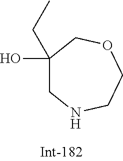 | 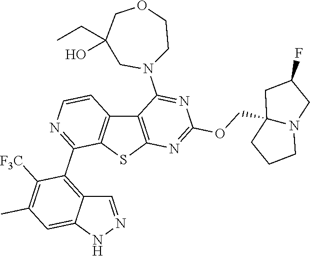 [SMILES: CCC1(O)COCCN(c2nc(OC[C@@]34CCCN3C[C@H](F)C4)nc3sc4c(-c5c(C(F)(F)F)c(C)cc6[nH]ncc56)nccc4c23)C1] | 6-ethyl-4-(2-(((2R,7aS)-2- fluorotetrahydro-1H-pyr- rolizin-7a(5H)-yl)meth- oxy)-8-(6-methyl-5-(tri- fluoromethyl)-1H-indazol- 4-yl)pyrido[4′,3′:4,5] thieno[2,3-d]pyrimidin-4- yl)-1,4-oxazepan-6-ol | 686 |
| Ex- 202 | 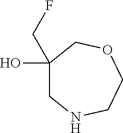 | 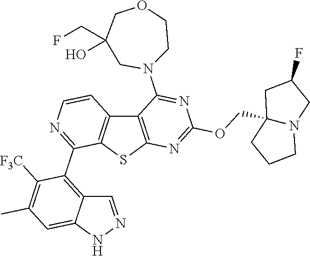 [SMILES: Cc1cc2[nH]ncc2c(-c2nccc3c2sc2nc(OC[C@@]45CCCN4C[C@H](F)C5)nc(N4CCOCC(O)(CF)C4)c23)c1C(F)(F)F] | 6-(fluoromethyl)-4-(2- (((2R,7aS)-2-fluorotetra- hydro-1H-pyrrolizin- 7a(5H)-yl)methoxy)-8- (6-methyl-5-(trifluoro- methyl)-1H-indazol-4-yl)- pyrido[4′,3′:4,5]thieno [2,3-d]pyrimidin-4-yl)- 1,4-oxazepan-6-ol | 690 |

### Example 203: (6S)-4-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidin-4-yl)-6-methoxy-6-methyl-1,4-oxazepane (Ex-203)

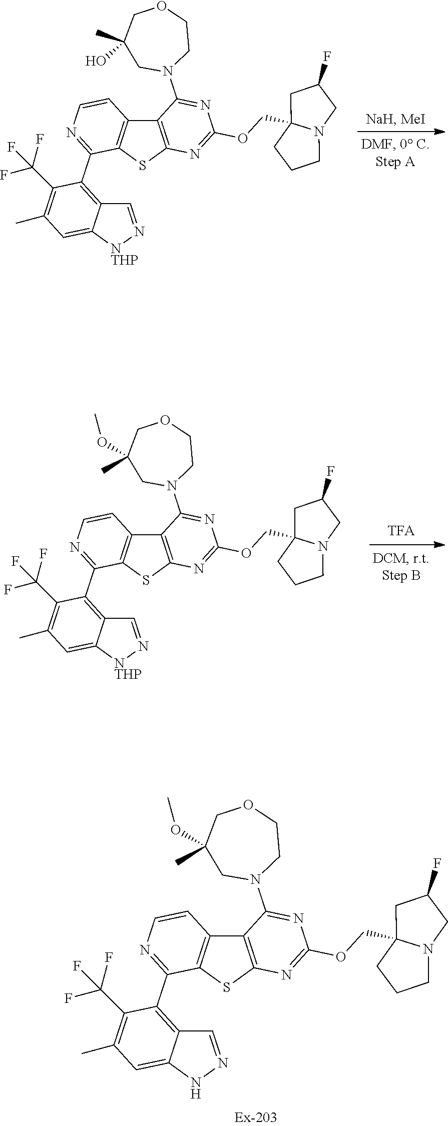

### Step A: (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidin-4-yl)-6-methoxy-6-methyl-1,4-oxazepane

NaH (2.1 mg, 0.05 mmol) and MeI (0.008 mL, 0.053 mmol) were added to a solution of (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidin-4-yl)-6-methyl-1,4-oxazepan-6-ol (from Step A of Example 34) (20 mg, 0.026 mmol) in DMF (0.26 mL) at 0° C. The mixture was stirred at 0° C. for 0.5 h. EtOAc, water, and sat. aq. NaHCO₃ were added and the organic phase was washed with water and brine, dried over sodium sulfate, filtered, and concentrated to give crude (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidin-4-yl)-6-methoxy-6-methyl-1,4-oxazepane. MS (ESI): m/z (M+H)⁺ 771.

### Step B: (6S)-4-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidin-4-yl)-6-methoxy-6-methyl-1,4-oxazepane (Ex-203)

TFA (0.1 mL, 1.3 mmol) was added to a solution of crude (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]thieno[2,3-d]pyrimidin-4-yl)-6-methoxy-6-methyl-1,4-oxazepane in CH₂Cl₂ (0.53 mL). The mixture was stirred at r.t. for 1.5 h. Water, sat. aq. NaHCO₃, and EtOAc were added at 0° C. and the organic phase was washed with sat. aq. NaHCO₃ and brine, dried over sodium sulfate, filtered, and concentrated. The residue was purified by preparative HPLC (H₂O:MeCN (0.1% formic acid)). Saturated sodium bicarbonate, and water were added to the purified fractions, followed by extraction with ethyl acetate. The extract was washed with a saturated sodium chloride solution and dried over sodium sulfate. The solvent was evaporated under reduced pressure, thereby obtaining (6S)-4-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]thieno[2,3-d]pyrimidin-4-yl)-6-methoxy-6-methyl-1,4-oxazepane (Ex-203). MS (ESI): m/z (M+H)⁺ 687. ¹H NMR (400 MHz, CDCl₃) δ 8.78-8.76 (m, 1H), 7.73-7.71 (m, 1H), 7.55-7.54 (brs, 1H), 7.45-7.44 (brs, 0.5H), 7.41-7.40 (brs, 0.5H), 5.36-5.23 (m, 1H), 4.31-4.04 (m, 6H), 3.83-3.25 (m, 7H), 3.08-2.98 (m, 1H), 2.93 (s, 1.5H), 2.73-2.72 (m, 4.5H), 2.34-2.18 (m, 3H), 2.06-1.95 (m, 3H), 1.08 (s, 1.5H), 1.06 (s, 1.5H).

### Example 204: (6S)-6-methyl-4-(8-(6-methyl-5-(trifluoromethyl)-1H-indazol-4-yl)-2-((S)-1-((S)-1-methylpyrrolidin-2-yl) ethoxy)pyrido[4′,3′:4,5]thieno[2,3-d]pyrimidin-4-yl)-1,4-oxazepan-6-ol (Ex-204)

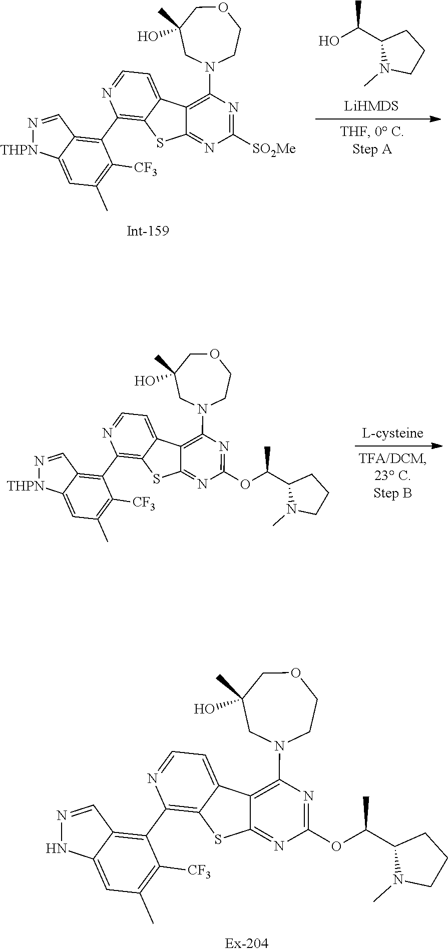

### Step A: (6S)-6-methyl-4-(8-(6-methyl-1-(tetrahydro-2H-pyran-2-yl)-5-(trifluoromethyl)-1H-indazol-4-yl)-2-((S)-1-((S)-1-methylpyrrolidin-2-yl)ethoxy)pyrido[4′,3′:4,5]thieno[2,3-d]pyrimidin-4-yl)-1,4-oxazepan-6-ol

A vial with stir bar was charged with (S)-1-((S)-1-methylpyrrolidin-2-yl) ethan-1-ol (13.02 mg, 0.101 mmol) and the reaction vessel was evacuated and backfilled with N₂ three times. THF (163 μl) was added into the reaction vessel and the resulting solution was cooled to 0° C. Lithium bis(trimethylsilyl)amide (98 μL, 0.098 mmol, 1 M in THF) was added into the reaction vessel and the resulting mixture was stirred for 30 min. This solution was added dropwise into a solution of (6S)-6-methyl-4-(8-(6-methyl-1-(tetrahydro-2H-pyran-2-yl)-5-(trifluoromethyl)-1H-indazol-4-yl)-2-(methylsulfonyl)pyrido[4′,3′:4,5]thieno[2,3-d]pyrimidin-4-yl)-1,4-oxazepan-6-ol (Int-159) (22 mg, 0.033 mmol) in THF (163 μL) cooled to 0° C. The resulting mixture was stirred for 50 min. The product mixture was diluted with ethyl acetate (2 mL) and sat. aq. sodium bicarbonate (2 mL). The layers were separated and the aqueous phase was extracted with EtOAc (2×2 mL). The combined organic layers were dried over sodium sulfate, filtered, and concentrated to obtain (6S)-6-methyl-4-(8-(6-methyl-1-(tetrahydro-2H-pyran-2-yl)-5-(trifluoromethyl)-1H-indazol-4-yl)-2-((S)-1-((S)-1-methylpyrrolidin-2-yl) ethoxy)pyrido[4′,3′:4,5]thieno[2,3-d]pyrimidin-4-yl)-1,4-oxazepan-6-ol, which was used without further purification. MS (ESI): (M+H)⁺ 726.

### Step B: (6S)-6-methyl-4-(8-(6-methyl-5-(trifluoromethyl)-1H-indazol-4-yl)-2-((S)-1-((S)-1-methylpyrrolidin-2-yl) ethoxy)pyrido[4′,3′:4,5]thieno[2,3-d]pyrimidin-4-yl)-1,4-oxazepan-6-ol (Ex-204)

A vial with a stir bar was charged with (6S)-6-methyl-4-(8-(6-methyl-1-(tetrahydro-2H-pyran-2-yl)-5-(trifluoromethyl)-1H-indazol-4-yl)-2-((S)-1-((S)-1-methylpyrrolidin-2-yl) ethoxy)pyrido[4′,3′:4,5]thieno[2,3-d]pyrimidin-4-yl)-1,4-oxazepan-6-ol (24.0 mg, 0.033 mmol) and L-cysteine (8.00 mg, 0.066 mmol). DCM (1.1 mL) and TFA (550 μL) were added and the reaction was allowed to stir at 0° C. for 70 min. The reaction mixture was added dropwise into sat. aq. NaHCO₃ (8 mL). The mixture was extracted with CHCl₃:iPrOH, 3:1 (3×6 mL). The combined organic phases were dried over sodium sulfate, filtered, and concentrated. The residue was purified by preparative reverse-phase HPLC (MeCN/H₂O w/NH₄OH as modifier) to give (6S)-6-methyl-4-(8-(6-methyl-5-(trifluoromethyl)-1H-indazol-4-yl)-2-((S)-1-((S)-1-methylpyrrolidin-2-yl) ethoxy)pyrido[4′,3′:4,5]thieno[2,3-d]pyrimidin-4-yl)-1,4-oxazepan-6-ol (Ex-204). MS (ESI): (M+H)⁺ 642. ¹H NMR (600 MHz, DMSO) δ 8.75 (d, J=5.5 Hz, 1H), 7.84 (d, J=5.5 Hz, 1H), 7.78 (s, 1H), 7.47 (d, J=7.1 Hz, 1H), 5.26-5.08 (m, 2H), 4.05-4.00 (m, 1H), 3.97-3.85 (m, 5H), 3.52 (dd, J=12.2, 7.9 Hz, 1H), 3.44 (d, J=12.2 Hz, 1H), 2.97-2.91 (m, 1H), 2.68 (s, 3H), 2.58-2.53 (m, 1H), 2.34 (d, J=1.8 Hz, 3H), 2.21-2.14 (m, 1H), 1.82-1.74 (m, 1H), 1.71-1.59 (m, 3H), 1.23 (dd, J=6.3, 3.4 Hz, 3H), 0.99 (d, J=11.7 Hz, 3H).

### Example 205: (6S)-4-(8-(5-cyclopropyl-6-(trifluoromethyl)-1H-indazol-4-yl)-2-(((4aS,7aR)-1-methyloctahydro-4aH-cyclopenta[b]pyridin-4a-yl)methoxy)pyrido[4′,3′:4,5]thieno[2,3-d]pyrimidin-4-yl)-6-methyl-1,4-oxazepan-6-ol (Ex-205)

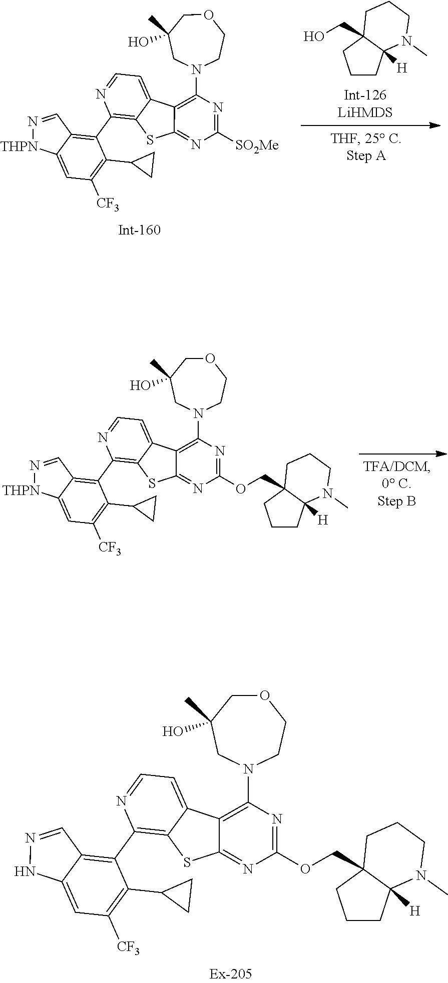

### Step A: (6S)-4-(8-(5-cyclopropyl-1-(tetrahydro-2H-pyran-2-yl)-6-(trifluoromethyl)-1H-indazol-4-yl)-2-(((4aS,7aR)-1-methyloctahydro-4aH-cyclopenta[b]pyridin-4a-yl)methoxy)pyrido[4′,3′:4,5]thieno[2,3-d]pyrimidin-4-yl)-6-methyl-1,4-oxazepan-6-ol

THF (711 μL) was added to a mixture of ((4aS,7aR)-1-methyloctahydro-4aH-cyclopenta[b]pyridin-4a-yl) methanol (Int-126) (23.3 mg, 128 μmol) and LiHMDS (256 μL, 256 μmol, 1 M in THF) under an atmosphere of N₂. The reaction was stirred for 1 h and to this was added (6S)-4-(8-(5-cyclopropyl-1-(tetrahydro-2H-pyran-2-yl)-6-(trifluoromethyl)-1H-indazol-4-yl)-2-(methylsulfonyl)pyrido[4′,3′:4,5]thieno[2,3-d]pyrimidin-4-yl)-6-methyl-1,4-oxazepan-6-ol (Int-160) (100 mg, 71.1 μmol). The reaction was stirred at room temperature for 17 h. The reaction was quenched with sat. NH₄Cl (1 mL) and extracted with EtOAc (3×1 mL). The combined organic layers washed with brine and concentrated. The crude product was purified by silica gel chromatography (0 to 100% EtOAc in hexanes) to provide (6S)-4-(8-(5-cyclopropyl-1-(tetrahydro-2H-pyran-2-yl)-6-(trifluoromethyl)-1H-indazol-4-yl)-2-(((4aS,7aR)-1-methyloctahydro-4aH-cyclopenta[b]pyridin-4a-yl)methoxy)pyrido[4′,3′:4,5]thieno[2,3-d]pyrimidin-4-yl)-6-methyl-1,4-oxazepan-6-ol. MS (ESI): m/z (M+H)⁺ 792.

### Step B (6S)-4-(8-(5-cyclopropyl-6-(trifluoromethyl)-1H-indazol-4-yl)-2-(4aS,7aR)-1-methyloctahydro-4aH-cyclopenta[b]pyridin-4a-yl)methoxy)pyrido[4′,3′:4,5]thieno[2,3-d]pyrimidin-4-yl)-6-methyl-1,4-oxazepan-6-ol (Ex-205)

DCM (505 μL) and TFA (195 μL, 2.53 mmol) were added to a vial containing (6S)-4-(8-(5-cyclopropyl-1-(tetrahydro-2H-pyran-2-yl)-6-(trifluoromethyl)-1H-indazol-4-yl)-2-(((4aS,7aR)-1-methyloctahydro-4aH-cyclopenta[b]pyridin-4a-yl)methoxy)pyrido[4′,3′:4,5]thieno[2,3-d]pyrimidin-4-yl)-6-methyl-1,4-oxazepan-6-ol (40 mg, 50.5 μmol) at 0° C. and stirred for 2 h. After 2 h, sat. aq. NaHCO₃ and DCM were added to the reaction and passed through a phase separator. The organic layer was concentrated in vacuo then purified via preparative reverse-phase HPLC (MeCN/H₂O w/NH₄OH modifier) to afford (6S)-4-(8-(5-cyclopropyl-6-(trifluoromethyl)-1H-indazol-4-yl)-2-(((4aS,7aR)-1-methyloctahydro-4aH-cyclopenta[b]pyridin-4a-yl)methoxy)pyrido[4′,3′:4,5]thieno[2,3-d]pyrimidin-4-yl)-6-methyl-1,4-oxazepan-6-ol (Ex-205). MS (ESI): m/z (M+H)⁺ 708. ¹H NMR (600 MHz, MeOD) δ 8.81 (d. J=5.5 Hz, 1H), 8.17 (s, 1H), 7.93 (d, J=5.5 Hz, 1H), 7.72 (d. J=12.8 Hz, 1H), 4.47 (d, J=10.6 Hz, 1H), 4.25 (d, J=10.5 Hz, 1H), 4.21-4.02 (m, 4H), 3.93 (dt, J=19.8, 10.0 Hz, 3H), 3.71 (d, J=12.6 Hz, 1H), 3.52 (d, J=12.5 Hz, 1H), 2.82 (s, 1H), 2.65 (s, 1H), 2.36 (s, 2H), 2.31 (s, 3H), 2.24 (s, 1H), 1.94 (s, 1H), 1.82 (s, 1H), 1.74 (d, J=7.7 Hz, 1H), 1.69 (d, J=9.3 Hz, 4H), 1.57 (d, J=8.9 Hz, 1H), 1.18 (s, 3H), 0.74 (d, J=4.7 Hz, 1H), 0.32 (dd, J=16.1, 7.0 Hz, 2H), 0.17 (s, 1H).

The example in the table below was synthesized using a similar procedure as described in the synthesis of Ex-205 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | | [M + H]⁺ |
|---|---|---|---|---|
| Ex. | Starting Material | Structure | Compound Name | Found |
| Ex- 206 | 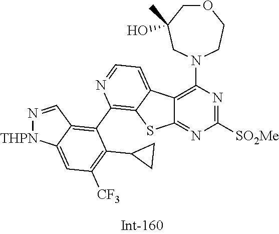 | 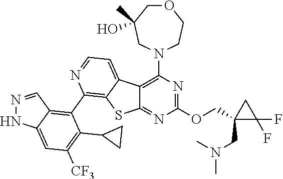 [SMILES: CN(C)C[C@@]1(COc2nc(N3CCOC[C@@](C)(O)C3)c3c(n2)sc2c(-c4c(C5CC5)c(C(F)(F)F)cc5[nH]ncc45)nccc23)CC1(F)F] | (6S)-4-(8-(5-cyclo- propyl-6-(trifluoro- methyl)-1H-inda- zol-4-yl)-2-(((R)-1- ((dimethylamino)- methyl)-2,2- difluorocycloprop- yl)methoxy)pyrido [4′,3′:4,5]thieno [2,3-d]pyrimidin- 4-yl)-6-methyl-1,4- oxazepan-6-ol | 704 |

### Example 207: ((3S,7aS)-7a-(((8-(5-cyclopropyl-6-(trifluoromethyl)-1H-indazol-4-yl)-4-((S)-6-hydroxy-6-methyl-1,4-oxazepan-4-yl)pyrido[4′,3′:4,5]thieno[2,3-d]pyrimidin-2-yl)oxy)methyl) hexahydro-1H-pyrrolizin-3-yl)methyl dimethylcarbamate (Ex-207)

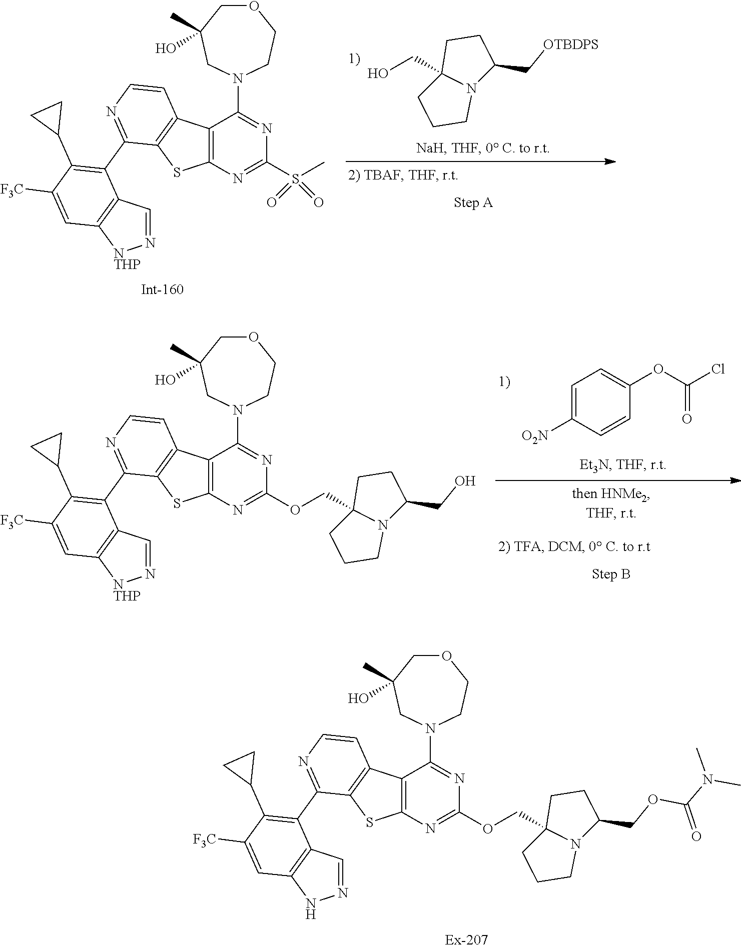

### Step A: (6S)-4-(8-(5-cyclopropyl-1-(tetrahydro-2H-pyran-2-yl)-6-(trifluoromethyl)-1H-indazol-4-yl)-2-(((3S,7aS)-3-(hydroxymethyl)tetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol

NaH (11.4 mg, 0.285 mmol, 60% dispersion in mineral oil) was added to a solution of (6S)-4-(8-(5-cyclopropyl-1-(tetrahydro-2H-pyran-2-yl)-6-(trifluoromethyl)-1H-indazol-4-yl)-2-(methylsulfonyl)pyrido[4′,3′:4,5]thieno[2,3-d]pyrimidin-4-yl)-6-methyl-1,4-oxazepan-6-ol (Int-160) (40.0 mg, 0.0569 mmol) and (3-((tert-butyl(diphenyl) silyl)oxymethyl)-1,2,3,5,6,7-hexahydropyrrolizin-8-yl]methanol (35.0 mg, 0.0599 mmol) in THF (1.14 mL) at 0° C. After warming to room temperature and stirring for 1 h, the reaction was quenched by the addition of H₂O. The reaction mixture was extracted with EtOAc, washed with brine, dried over Na₂SO₄, filtered and concentrated in vacuo. The residue was purified by flash NH-silica gel chromatography (0 to 20%, MeOH in CHCl₃) to afford (6S)-4-(2-(((3S,7aS)-3-(((tert-butyldiphenylsilyl)oxy)methyl)tetrahydro-1H-pyrrolizin-7a(5H)-yl)methoxy)-8-(5-cyclopropyl-1-(tetrahydro-2H-pyran-2-yl)-6-(trifluoromethyl)-1H-indazol-4-yl)pyrido[4′,3′:4,5]thieno[2,3-d]pyrimidin-4-yl)-6-methyl-1,4-oxazepan-6-ol.

A solution of TBAF (0.285 mL, 0.285 mmol, 1 M in THF) was added to a solution of (6S)-4-(2-(((3S,7aS)-3-(((tert-butyldiphenylsilyl)oxy)methyl)tetrahydro-1H-pyrrolizin-7a(5H)-yl)methoxy)-8-(5-cyclopropyl-1-(tetrahydro-2H-pyran-2-yl)-6-(trifluoromethyl)-1H-indazol-4-yl)pyrido[4′,3′:4,5]thieno[2,3-d]pyrimidin-4-yl)-6-methyl-1,4-oxazepan-6-ol in THF (0.57 mL) at room temperature. After stirring the mixture at room temperature for 15 h, the reaction was diluted with EtOAc, washed with phosphate buffer solution and brine, dried over Na₂SO₄, filtered, and concentrated in vacuo to afford (6S)-4-(8-(5-cyclopropyl-1-(tetrahydro-2H-pyran-2-yl)-6-(trifluoromethyl)-1H-indazol-4-yl)-2-(((3S,7aS)-3-(hydroxymethyl)tetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol. ESI-MS m/z [M+H]⁺ 794.

### Step B: ((3S,7aS)-7a-(((8-(5-cyclopropyl-6-(trifluoromethyl)-1H-indazol-4-yl)-4-((S)-6-hydroxy-6-methyl-1,4-oxazepan-4-yl)pyrido[4′,3′:4,5]thieno[2,3-d]pyrimidin-2-yl)oxy)methyl)hexahydro-1H-pyrrolizin-3-yl)methyl dimethylcarbamate (Ex-207)

4-nitrophenyl chloroformate (8.1 mg, 0.0605 mmol) was added to a solution of (6S)-4-(8-(5-cyclopropyl-1-(tetrahydro-2H-pyran-2-yl)-6-(trifluoromethyl)-1H-indazol-4-yl)-2-(((3S,7aS)-3-(hydroxymethyl)tetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (30.0 mg, 0.0378 mmol) and triethylamine (0.0263 mL, 0.189 mmol) in THF (0.38 mL) at room temperature. After stirring the mixture at room temperature for 40 min, to the reaction mixture was added dimethylamine (0.567 mL, 1.13 mmol, 2 M in THF). After stirring the mixture at room temperature for 5 min, the reaction was quenched by the addition of sat. aq. NaHCO₃. The reaction mixture was extracted with EtOAc, washed with brine, dried over Na₂SO₄, filtered, and concentrated in vacuo to afford ((3S,7aS)-7a-(((8-(5-cyclopropyl-1-(tetrahydro-2H-pyran-2-yl)-6-(trifluoromethyl)-1H-indazol-4-yl)-4-((S)-6-hydroxy-6-methyl-1,4-oxazepan-4-yl)pyrido[4′,3′:4,5]thieno[2,3-d]pyrimidin-2-yl)oxy)methyl) hexahydro-1H-pyrrolizin-3-yl)methyl dimethylcarbamate, which was used directly in the next step without further purification.

TFA (0.378 mL, 5.09 mmol) was added to a solution of ((3S,7aS)-7a-(((8-(5-cyclopropyl-1-(tetrahydro-2H-pyran-2-yl)-6-(trifluoromethyl)-1H-indazol-4-yl)-4-((S)-6-hydroxy-6-methyl-1,4-oxazepan-4-yl)pyrido[4′,3′:4,5]thieno[2,3-d]pyrimidin-2-yl)oxy)methyl) hexahydro-1H-pyrrolizin-3-yl)methyl dimethylcarbamate in DCM (0.76 mL) at 0° C. The mixture was warmed to r.t. and stirred for 30 min, the reaction was diluted with CHCl₃ and quenched by the addition of sat. aq. NaHCO₃. The mixture was extracted with CHCl₃, washed with brine, dried over Na₂SO₄, filtered, and concentrated in vacuo. The residue was purified by preparative reverse-phase HPLC (MeCN/H₂O w/formic acid modifier) to afford ((3S,7aS)-7a-(((8-(5-cyclopropyl-6-(trifluoromethyl)-1H-indazol-4-yl)-4-((S)-6-hydroxy-6-methyl-1,4-oxazepan-4-yl)pyrido[4′,3′:4,5]thieno[2,3-d]pyrimidin-2-yl)oxy)methyl) hexahydro-1H-pyrrolizin-3-yl)methyl dimethylcarbamate (Ex-207). ESI-MS m/z [M+H]⁺ 781. ¹H-NMR (400 MHz, do-DMSO) δ 13.65 (s, 1H), 8.87-8.78 (m, 1H), 8.10 (s, 1H), 7.85 (d, J=6.5 Hz, 1H), 7.70 (s, 1H), 5.27-4.99 (m, 1H), 4.24-3.80 (m, 9H), 3.62-3.35 (m, 5H), 2.84-2.75 (m, 6H), 2.74-2.64 (m, 1H), 2.57-2.52 (m, 1H), 2.19-1.93 (m, 2H), 1.80-1.42 (m, 6H), 1.02-0.90 (m, 3H), 0.71-0.50 (m, 1H), 0.36-0.03 (3H).

### Example 208: (6S)-4-(8-(6-chloro-5-cyclopropyl-1H-indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-208)

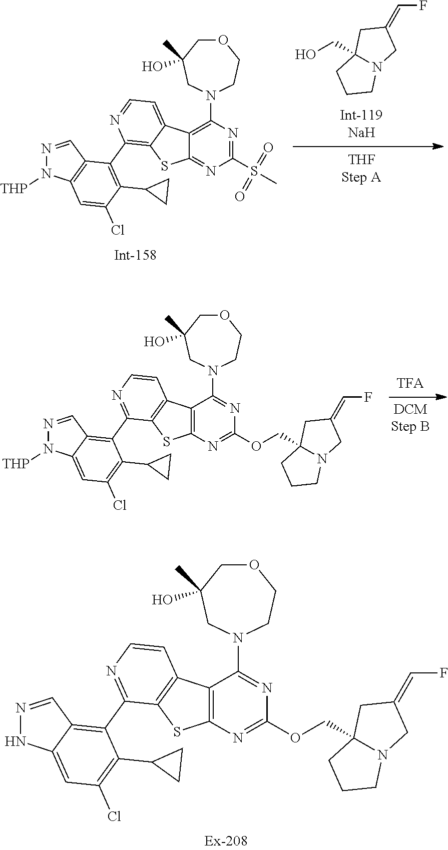

### Step A: (6S)-4-(8-(6-chloro-5-cyclopropyl-1-(tetrahydro-2H-pyran-2-yl)-1H-indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol

NaH (12 mg, 0.3 mmol, 60 wt % in mineral oil) was added to a solution of (6S)-4-(8-(6-chloro-5-cyclopropyl-1-(tetrahydro-2H-pyran-2-yl)-1H-indazol-4-yl)-2-(methylsulfonyl)pyrido[4′,3′:4,5]thieno[2,3-d]pyrimidin-4-yl)-6-methyl-1,4-oxazepan-6-ol (Int-158) (40 mg, 0.060 mmol) and (S,Z)-(2-(fluoromethylene)tetrahydro-1H-pyrrolizin-7a(5H)-yl) methanol (Int-119) (10.2 mg, 0.06 mmol) in dry THF (2 mL) at 0° C. The reaction solution was warmed to 25° C. and stirred for 10 min. The mixture was quenched with water (3 mL) and extracted with EtOAc (2×10 mL). The combined organic layers were dried over sodium sulfate, filtered, and the solvent was evaporated under reduced pressure. The crude was purified by preparative TLC (SiO₂, DCM/MeOH=15:1) to give (6S)-4-(8-(6-chloro-5-cyclopropyl-1-(tetrahydro-2H-pyran-2-yl)-1H-indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol. MS (ESI) [M+H]⁺: m/z 760.

### Step B: (6S)-4-(8-(6-chloro-5-cyclopropyl-1H-indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-208)

TFA (0.5 mL) was added to a solution of (6S)-4-(8-(6-chloro-5-cyclopropyl-1-(tetrahydro-2H-pyran-2-yl)-1H-indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (30 mg, 0.039 mmol) in DCM (0.5 mL) at 25° C. and the mixture was stirred for 5 min. The solvent was removed under reduced pressure and the residue was diluted with EtOAc (15 mL) and sat. aq. NaHCO₃ (2 mL). The organic layer was separated and dried over sodium sulfate, filtered, and concentrated in vacuo. The crude product was purified by preparative reverse-phase HPLC (MeCN/H₂O w/NH₄HCO; modifier) to give (6S)-4-(8-(6-chloro-5-cyclopropyl-1H-indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-208). MS (ESI) [M+H]⁺: m/z 676. ¹H NMR (400 MHz, CD₃OD) δ 8.65 (dd, J=1.31, 5.60 Hz, 1H), 7.79 (dd, J=1.79, 5.60 Hz, 1H), 7.71 (s, 1H), 7.50 (d, J=10.01 Hz, 1H), 6.36-6.63 (m, 1H), 4.20 (dd, J=4.29, 10.49 Hz, 1H), 3.73-4.10 (m, 7H), 3.68 (br d, J=13.59 Hz, 1H), 3.57 (dd, J=7.87, 12.52 Hz, 1H), 3.30-3.41 (m, 2H), 3.01 (s, 1H), 2.51-2.61 (m, 2H), 2.30 (br d, J=15.50 Hz, 1H), 1.69-1.98 (m, 5H), 1.00-1.10 (m, 3H), 0.54-0.71 (m, 1H), 0.23-0.38 (m, 1H), −0.09-−0.06 (m, 2H).

The examples in the table below were synthesized using a similar procedure as described in the synthesis of Ex-208 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | | SFC | |
|---|---|---|---|---|---|
| | | | | Separation | [M + H]⁺ |
| Ex. | Starting Material | Structure | Compound Name | Conditions | Found |
| Ex- 209 | 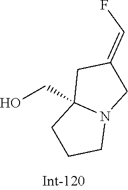 [SMILES: OC[C@@]12CCCN1C/C(=C/F)C2] | 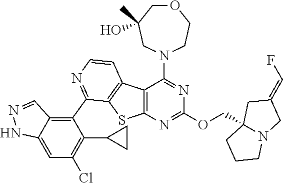 [SMILES: C[C@@]1(O)COCCN(c2nc(OC[C@@]34CCCN3C/C(=C/F)C4)nc3sc4c(-c5c(C6CC6)c(Cl)cc6[nH]ncc56)nccc4c23)C1] | (6S)-4-(8-(6-chloro- 5-cyclopropyl-1H- indazol-4-yl)-2- (((S,E)-2-(fluoro- methylene)tetra- hydro-1H-pyrrol- izin-7a(5H)-yl)- methoxy)pyrido [4′,3′:4,5]thieno [2,3-d]pyrimidin- 4-yl)-6-methyl- 1,4-oxazepan-6-ol | | 676 |
| Ex- 210 | 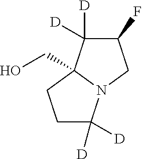 [SMILES: [2H]C1([2H])CC[C@]2(CO)N1C[C@H](F)C2([2H])[2H]] | 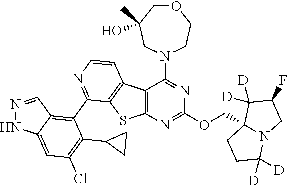 [SMILES: [2H]C1([2H])CC[C@]2(COc3nc(N4CCOC[C@@](C)(O)C4)c4c(n3)sc3c(-c5c(C6CC6)c(Cl)cc6[nH]ncc56)nccc34)N1C[C@H](F)C2([2H])[2H]] | (6S)-4-(8-(6-chloro- 5-cyclopropyl-1H- indazol-4-yl)-2- (((2R,7aS)-2-fluoro- tetrahydro-1H- pyrrolizin-7a(5H)- yl-1,1,5,5-d₄)meth- oxy)pyrido[4′,3′: 4,5]thieno[2,3-d] pyrimidin-4-yl)-6- methyl-1,4-oxaze- pan-6-ol | | 668 |
| Ex- 211 | 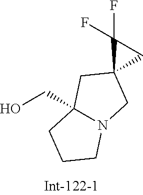 [SMILES: OC[C@@]12CCCN1C[C@@]1(CC1(F)F)C2] | 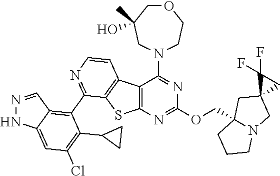 [SMILES: C[C@@]1(O)COCCN(c2nc(OC[C@@]34CCCN3C[C@@]3(CC3(F)F)C4)nc3sc4c(-c5c(C6CC6)c(Cl)cc6[nH]ncc56)nccc4c23)C1] | (6S)-4-(8-(6-chloro- 5-cyclopropyl-1H- indazol-4-yl)-2- (((1S,7a′S)-2,2- difluorodihydro- 1′H,3′H-spiro[cyclo- propane-1,2′-pyrrol- izin]-7a′(5′H)-yl)- methoxy)pyrido [4′,3′:4,5]thieno[2,3- d]pyrimidin-4-yl)-6- methyl-1,4-oxaze- pan-6-ol | | 708 |
| Ex- 212 | 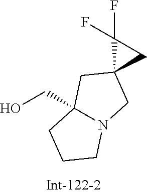 [SMILES: OC[C@@]12CCCN1C[C@]1(CC1(F)F)C2] | 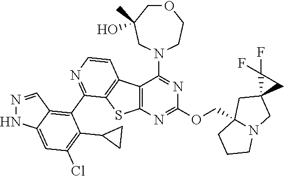 [SMILES: C[C@@]1(O)COCCN(c2nc(OC[C@@]34CCCN3C[C@]3(CC3(F)F)C4)nc3sc4c(-c5c(C6CC6)c(Cl)cc6[nH]ncc56)nccc4c23)C1] | (6S)-4-(8-(6-chloro- 5-cyclopropyl-1H- indazol-4-yl)-2- (((1R,7a'S)-2,2- difluorodihydro- 1′H,3′H-spiro[cyclo- propane-1,2′-pyrrol- izin]-7a′(5′H)-yl)- methoxy)pyrido [4′,3′:4,5]thieno[2,3- d]pyrimidin-4-yl)-6- methyl-1,4-oxaze- pan-6-ol | | 708 |
| Ex- 213 | 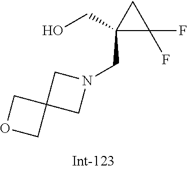 [SMILES: OC[C@]1(CN2CC3(COC3)C2)CC1(F)F] | 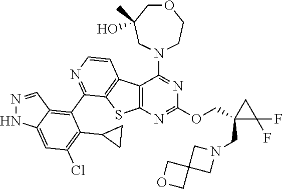 [SMILES: C[C@@]1(O)COCCN(c2nc(OC[C@]3(CN4CC5(COC5)C4)CC3(F)F)nc3sc4c(-c5c(C6CC6)c(Cl)cc6[nH]ncc56)nccc4c23)C1] | (6S)-4-(2-(((R)-1- ((2-oxa-6-azaspiro [3.3]heptan-6-yl)- methyl)-2,2-difluoro- cyclopropyl)meth- oxy)-8-(6-chloro-5- cyclopropyl-1H- indazol-4-yl)pyrido [4′,3′:4,5]thieno [2,3-d]pyrimidin-4- yl)-6-methyl-1,4- oxazepan-6-ol | | 724 |
| Ex- 214 | 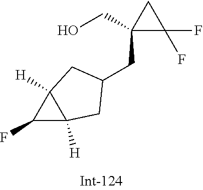 [SMILES: OC[C@]1(CC2C[C@@H]3[C@H](F)[C@@H]3C2)CC1(F)F] | 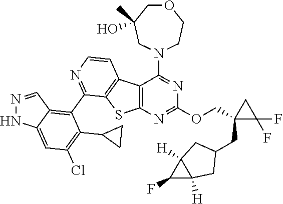 [SMILES: C[C@@]1(O)COCCN(c2nc(OC[C@]3(CC4C[C@@H]5[C@H](F)[C@@H]5C4)CC3(F)F)nc3sc4c(-c5c(C6CC6)c(Cl)cc6[nH]ncc56)nccc4c23)C1] | (6S)-4-(8-(6-chloro- 5-cyclopropyl-1H- indazol-4-yl)-2- (((R)-2,2-difluoro- 1-(((1R,5S,6R)-6- fluoro-3-azabicyclo [3.1.0]hexan-3-yl)- methyl)cycloprop- yl)methoxy)pyrido [4′,3′:4,5]thieno[2,3- d]pyrimidin-4-yl)-6- methyl-1,4-oxaze- pan-6-ol | | 726 |
| Ex- 215 | 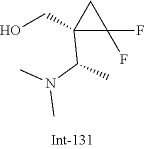 | 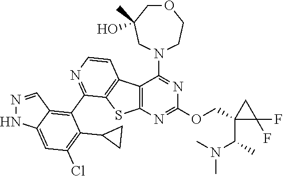 [SMILES: C[C@H](N(C)C)[C@@]1(COc2nc(N3CCOC[C@@](C)(O)C3)c3c(n2)sc2c(-c4c(C5CC5)c(Cl)cc5[nH]ncc45)nccc23)CC1(F)F] | (6S)-4-(8-(6-chloro- 5-cyclopropyl-1H- indazol-4-yl)-2- (((R)-1-((S)-1- (dimethylamino)- ethyl)-2,2-difluoro- cyclopropyl)meth- oxy)pyrido[4′,3′: 4,5]thieno[2,3-d] pyrimidin-4-yl)-6- methyl-1,4-oxaze- pan-6-ol | | 685 |
| Ex- 216 | 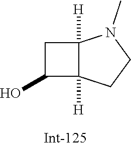 | 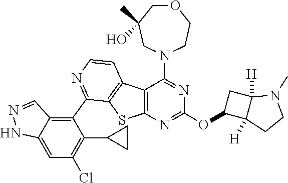 | (6S)-4-(8-(6-chloro- 5-cyclopropyl-1H- indazol-4-yl)-2- (((1R,5R,6S)-2- methyl-2-azabicyclo [3.2.0]heptan-6-yl)- oxy)pyrido[4′,3′: 4,5]thieno[2,3-d] pyrimidin-4-yl)-6- methyl-1,4-oxaze- pam-6-ol | | 632 |
| Ex- 217 | 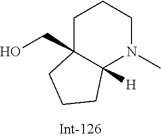 | 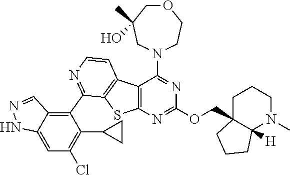 [SMILES: CN1CCC[C@@]2(COc3nc(N4CCOC[C@@](C)(O)C4)c4c(n3)sc3c(-c5c(C6CC6)c(Cl)cc6[nH]ncc56)nccc34)CCC[C@@H]12] | (6S)-4-(8-(6-chloro- 5-cyclopropyl-1H- indazol-4-yl)-2- (((4aS,7aR)-1-meth- yloctahydro-4aH- cyclopenta[b] pyridin-4a-yl)meth- oxy)pyrido[4′,3′:4,5] thieno[2,3-d]pyrimi- din-4-yl)-6-methyl- 1,4-oxazepan-6-ol | | 674 |
| Ex- 218 | 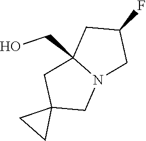 [SMILES: OC[C@@]12C[C@@H](F)CN1CC1(CC1)C2] | 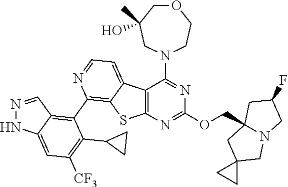 [SMILES: C[C@@]1(O)COCCN(c2nc(OC[C@@]34C[C@@H](F)CN3CC3(CC3)C4)nc3sc4c(-c5c(C6CC6)c(C(F)(F)F)cc6[nH]ncc56)nccc4c23)C1] | (6S)-4-(8-(6-chloro- 5-cyclopropyl-1H- indazol-4-yl)-2- (((6'R,7a′R)-6′- fluorodihydro- 1′H,3′H-spiro[cyclo- propane-1,2′-pyr- rolizin]-7a′(5′H)- yl)methoxy)pyrido [4′,3′:4,5]thieno[2,3- d]pyrimidin-4-yl)-6- methyl-1,4-oxaze- pan-6-ol | | 690 |
| Ex- 219 | 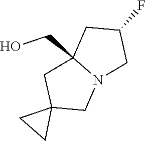 [SMILES: OC[C@@]12C[C@H](F)CN1CC1(CC1)C2] | 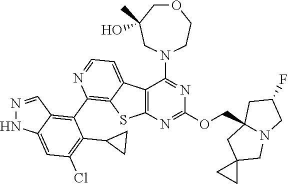 [SMILES: C[C@@]1(O)COCCN(c2nc(OC[C@@]34C[C@H](F)CN3CC3(CC3)C4)nc3sc4c(-c5c(C6CC6)c(Cl)cc6[nH]ncc56)nccc4c23)C1] | (6S)-4-(8-(6-chloro- 5-cyclopropyl-1H- indazol-fluoro- dihydro-1H,3′H- spiro[cyclopropane- 1,2′-yl)methoxy)- pyrido[4′,3′:4,5] thieno[2,3-d]pyr- imidin-4-yl)-6- methyl-1,4-oxaze- pan-6-ol | | 690 |
| Ex- 220 | 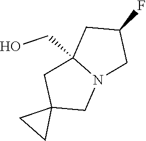 [SMILES: OC[C@]12C[C@@H](F)CN1CC1(CC1)C2] | 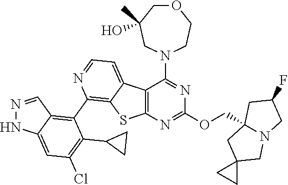 [SMILES: C[C@@]1(O)COCCN(c2nc(OC[C@]34C[C@@H](F)CN3CC3(CC3)C4)nc3sc4c(-c5c(C6CC6)c(Cl)cc6[nH]ncc56)nccc4c23)C1] | (6S)-4-(8-(6-chloro- 5-cyclopropyl-1H- indazol-4-yl)-2- (((6R,7a′S)-6′- fluorodihydro-1H, 3H-spiro[cyclopro- pane-1,2′-pyrrol- izin]7a′(5′H)-yl)- methoxy)pyrido [4′,3′:4,5]thieno[2,3- d]pyrimidin-4-yl)-6- methyl-1,4-oxaze- pan-6-ol | | 690 |
| Ex- 221 | 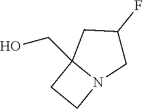 | 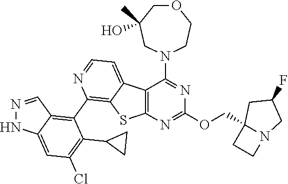 [SMILES: C[C@@]1(O)COCCN(c2nc(OC[C@@]34CCN3C[C@H](F)C4)nc3sc4c(-c5c(C6CC6)c(Cl)cc6[nH]ncc56)nccc4c23)C1] | (6S)-4-(8-(6-chloro- 5-cyclopropyl-1H- indazol-4-yl)-2- (((3R,5S)-3-fluoro- 1-azabicyclo[3.2.0] heptan-5-yl)meth- oxy)pyrido[4′,3′: 4,5]thieno[2,3-d] pyrimidin-4-yl)-6- methyl-1,4-oxaze- pan-6-ol | Prep reverse- phase HPLC (MeCN/H₂O w/TFA modifier) second eluting peak Then Column I, 25% MeOH w/0.1% NH₄OH, first eluting peak | 650 |
| Ex- 222 | 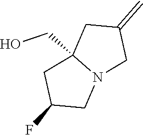 | 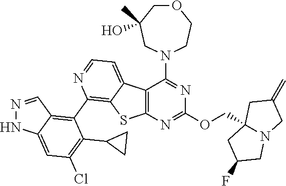 [SMILES: C=C1CN2C[C@@H](F)C[C@@]2(COc2nc(N3CCOC[C@@](C)(O)C3)c3c(n2)sc2c(-c4c(C5CC5)c(Cl)cc5[nH]ncc45)nccc23)C1] | (6S)-4-(8-(6-chloro- 5-cyclopropyl-1H- indazol-4-yl)-2- (((2S,7aR)-2-fluoro- 6-methylenetetra- hydro-1H-pyrrol- izin-7a(5H)-yl) methoxy)pyrido [4′,3′:4,5]thieno[2,3- d]pyrimidin-4-yl)-6- methyl-1,4-oxaze- pan-6-ol | | 676 |
| Ex- 223 | 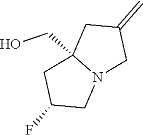 | 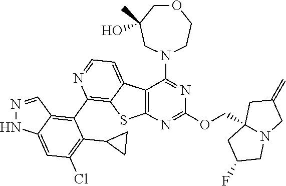 [SMILES: C=C1CN2C[C@H](F)C[C@@]2(COc2nc(N3CCOC[C@@](C)(O)C3)c3c(n2)sc2c(-c4c(C5CC5)c(Cl)cc5[nH]ncc45)nccc23)C1] | (6S)-4-(8-(6-chloro- 5-cyclopropyl-1H- indazol-4-yl)-2- (((2R,7aR)-2-fluoro- 6-methylenetetra- hydro-1H-pyrrol- izin-7a(5H)-yl) methoxy)pyrido [4′,3′:4,5]thieno[2,3- d]pyrimidin-4-yl)-6- methyl-1,4-oxaze- pan-6-ol | | 676 |
| Ex- 224 | 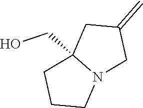 | 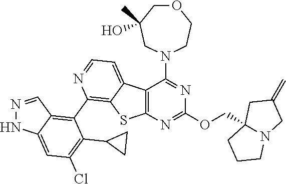 [SMILES: C=C1CN2CCC[C@@]2(COc2nc(N3CCOC[C@@](C)(O)C3)c3c(n2)sc2c(-c4c(C5CC5)c(Cl)cc5[nH]ncc45)nccc23)C1] | (6S)-4-(8-(6-chloro- 5-cyclopropyl-1H- indazol-4-yl)-2- (((S)-2-methylene- tetrahydro-1H- pyrrolizin-7a(5H)- yl)methoxy)pyrido [4′,3′:4,5]thieno[2,3- d]pyrimidin-4-yl)-6- methyl-1,4-oxaze- pan-6-ol | | 658 |
| Ex- 225 | 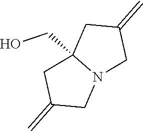 | 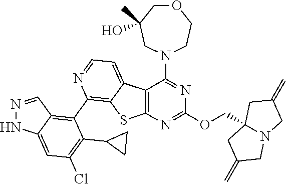 | (6S)-4-(8-(6-chloro- 5-cyclopropyl-1H- indazol-4-yl)-2- ((2,6-dimethylene- tetrahydro-1H- pyrrolizin-7a(5H)- yl)methoxy)pyrido [4′,3′:4,5]thieno[2,3- d]pyrimidin-4-yl)-6- methyl-1,4-oxaze- pan-6-ol | | 671 |
| Ex- 226 | 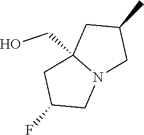 | 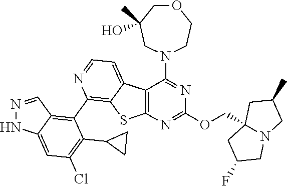 [SMILES: C[C@H]1CN2C[C@H](F)C[C@@]2(COc2nc(N3CCOC[C@@](C)(O)C3)c3c(n2)sc2c(-c4c(C5CC5)c(Cl)cc5[nH]ncc45)nccc23)C1] | (6S)-4-(8-(6-chloro- 5-cyclopropyl-1H- indazol-4-yl)-2- (((2R,6R,7aR)-2- fluoro-6-methyl- tetrahydro-1H- pyrrolizin-7a(5H)- yl)methoxy)pyrido [4′,3′:4,5]thieno[2,3- d]pyrimidin-4-yl)-6- methyl-1,4-oxaze- pan-6-ol | | 678 |
| Ex- 227 | 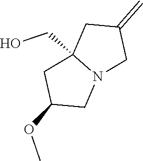 | 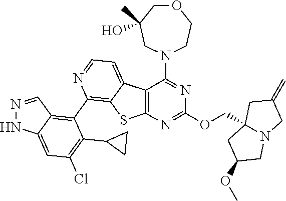 [SMILES: C=C1CN2C[C@@H](OC)C[C@@]2(COc2nc(N3CCOC[C@@](C)(O)C3)c3c(n2)sc2c(-c4c(C5CC5)c(Cl)cc5[nH]ncc45)nccc23)C1] | (6S)-4-(8-(6-chloro- 5-cyclopropyl-1H- indazol-4-yl)-2- (((25,7aR)-2-meth- oxy-6-methylene- tetrahydro-1H- pyrrolizin-7a(5H)- yl)methoxy)pyrido [4′,3′:4,5]thieno[2,3- d]pyrimidin-4-yl)-6- methyl-1,4-oxaze- pan-6-ol | | 689 |
| Ex- 228 | 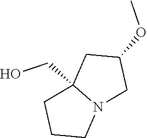 | 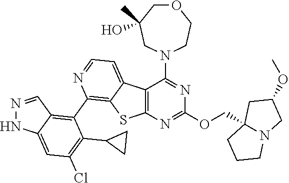 [SMILES: CO[C@@H]1CN2CCC[C@@]2(COc2nc(N3CCOC[C@@](C)(O)C3)c3c(n2)sc2c(-c4c(C5CC5)c(Cl)cc5[nH]ncc45)nccc23)C1] | (6S)-4-(8-(6-chloro- 5-cyclopropyl-1H- indazol-4-yl)-2- (((25,7aS)-2-meth- oxytetrahydro-1H- pyrrolizin-7a(5H)- yl)methoxy)pyrido [4′,3′:4,5]thieno[2,3- d]pyrimidin-4-yl)-6- methyl-1,4-oxaze- pan-6-ol | | 676 |
| Ex- 229 | 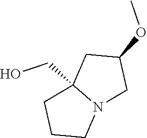 | 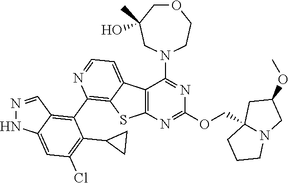 [SMILES: CO[C@H]1CN2CCC[C@@]2(COc2nc(N3CCOC[C@@](C)(O)C3)c3c(n2)sc2c(-c4c(C5CC5)c(Cl)cc5[nH]ncc45)nccc23)C1] | (6S)-4-(8-(6-chloro- 5-cyclopropyl-1H- indazol-4-yl)-2- (((2R,7aS)-2-meth- oxytetrahydro-1H- pyrrolizin-7a(5H)- yl)methoxy)pyrido [4′,3′:4,5]thieno[2,3- d]pyrimidin-4-yl)-6- methyl-1,4-oxaze- pan-6-ol | | 676 |

### Example 230: (6S)-4-(8-(6-chloro-5-cyclopropyl-1H-indazol-4-yl)-2-(R)-2,2-difluoro-1-((6-fluoro-2-azaspiro[3,3]heptan-2-yl)methyl)cyclopropyl)methoxy)pyrido[4′,3′:4,5]thieno[2,3-d]pyrimidin-4-yl)-6-methyl-1,4-oxazepan-6-ol (Ex-230)

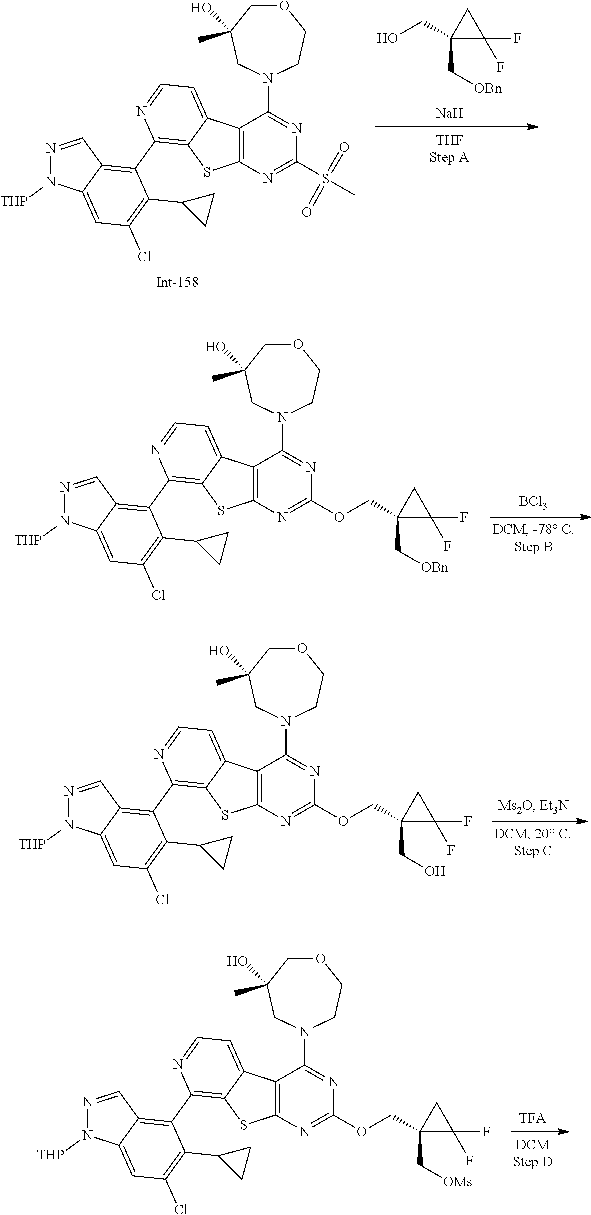

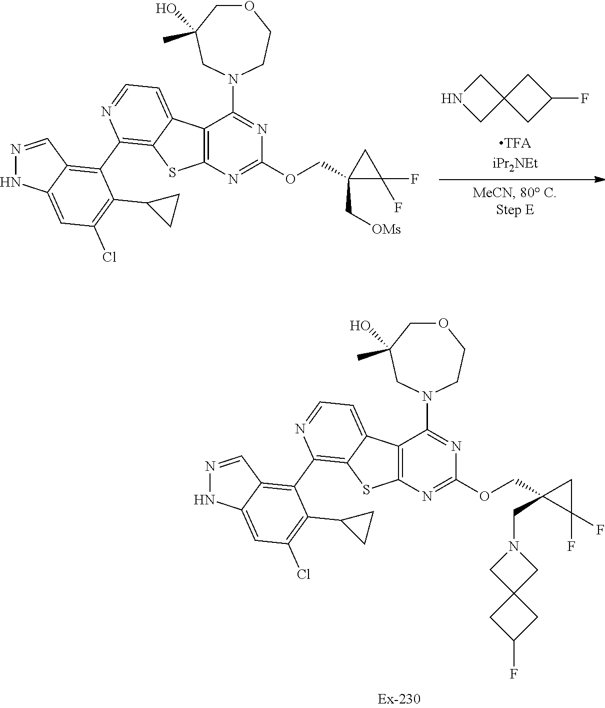 [SMILES: C[C@@]1(O)COCCN(c2nc(OC[C@]3(COS(C)(=O)=O)CC3(F)F)nc3sc4c(-c5c(C6CC6)c(Cl)cc6[nH]ncc56)nccc4c23)C1 ; FC1CC2(CNC2)C1 ; C[C@@]1(O)COCCN(c2nc(OC[C@]3(CN4CC5(CC(F)C5)C4)CC3(F)F)nc3sc4c(-c5c(C6CC6)c(Cl)cc6[nH]ncc56)nccc4c23)C1]

### Step A: (6S)-4-(2-(((R)-1-((benzyloxy)methyl)-2,2-difluorocyclopropyl)methoxy)-8-(6-chloro-5-cyclopropyl-1-(tetrahydro-2H-pyran-2-yl)-1H-indazol-4-yl)pyrido[4′,3′:4,5]thieno[2,3-d]pyrimidin-4-yl)-6-methyl-1,4-oxazepan-6-ol

To a solution of (6S)-4-(8-(6-chloro-5-cyclopropyl-1-(tetrahydro-2H-pyran-2-yl)-1H-indazol-4-yl)-2-(methylsulfonyl)pyrido[4′,3′:4,5]thieno[2,3-d]pyrimidin-4-yl)-6-methyl-1,4-oxazepan-6-ol (Int-158) (150 mg, 0.22 mmol) and(S)-(1-((benzyloxy)methyl)-2,2-difluorocyclopropyl) methanol (77 mg, 0.34 mmol) in THF (2.0 mL) was added NaH (45 mg, 1.12 mmol, 60% in mineral oil) at 0° C. under N₂. The reaction mixture was stirred at 0° C. for 30 min. The reaction solution was quenched with sat. aq. NH₄Cl (0.5 mL), extracted with EtOAc (3×2 mL). The combined organic layers were washed with brine (2 mL), dried over sodium sulfate, filtered, and the solvent was evaporated under reduced pressure to give the crude product. The crude product was purified by preparative TLC (SiO₂, 100% EtOAc) to give (6S)-4-(2-(((R)-1-((benzyloxy)methyl)-2,2-difluorocyclopropyl)methoxy)-8-(6-chloro-5-cyclopropyl-1-(tetrahydro-2H-pyran-2-yl)-1H-indazol-4-yl)pyrido[4′,3′:4,5]thieno[2,3-d]pyrimidin-4-yl)-6-methyl-1,4-oxazepan-6-ol. MS (ESI) [M+H]⁺: m/z 817.

### Step B: (6S)-4-(8-(6-chloro-5-cyclopropyl-1-(tetrahydro-2H-pyran-2-yl)-1H-indazol-4-yl)-2-(((R)-2,2-difluoro-1-(hydroxymethyl)cyclopropyl)methoxy)pyrido[4′,3′:4,5]thieno[2,3-d]pyrimidin-4-yl)-6-methyl-1,4-oxazepan-6-ol

To a solution of (6S)-4-(2-(((R)-1-((benzyloxy)methyl)-2,2-difluorocyclopropyl)methoxy)-8-(6-chloro-5-cyclopropyl-1-(tetrahydro-2H-pyran-2-yl)-1H-indazol-4-yl)pyrido[4′,3′:4,5]thieno[2,3-d]pyrimidin-4-yl)-6-methyl-1,4-oxazepan-6-ol (76 mg, 0.093 mmol) in DCM (2 mL) was added boron trichloride (0.93 mL, 0.93 mmol, 1.0 M in DCM) at −78° C. under N₂. The reaction mixture was stirred at −78° C. for 1 h. The reaction solution was quenched with MeOH (0.5 mL) at −78° C., poured into sat. aq. NaHCO₃. The mixture was extracted with DCM (5 mL) and the combined organic layers were dried over sodium sulfate, filtered, and the solvent was evaporated under reduced pressure. The crude product was purified by preparative TLC (SiO₂, DCM/MeOH=20:1) to give (6S)-4-(8-(6-chloro-5-cyclopropyl-1-(tetrahydro-2H-pyran-2-yl)-1H-indazol-4-yl)-2-(((R)-2,2-difluoro-1-(hydroxymethyl)cyclopropyl)methoxy)pyrido[4′,3′:4,5]thieno[2,3-d]pyrimidin-4-yl)-6-methyl-1,4-oxazepan-6-ol. MS (ESI) [M+H]⁺: m/z 727.

### Step C: ((1S)-1-(((8-(6-chloro-5-cyclopropyl-1-(tetrahydro-2H-pyran-2-yl)-1H-indazol-4-yl)-4-((S)-6-hydroxy-6-methyl-1,4-oxazepan-4-yl)pyrido[4′,3′:4,5]thieno[2,3-d]pyrimidin-2-yl)oxy)methyl)-2,2-difluorocyclopropyl)methyl methanesulfonate

To a stirred solution of (6S)-4-(8-(6-chloro-5-cyclopropyl-1-(tetrahydro-2H-pyran-2-yl)-1H-indazol-4-yl)-2-(((R)-2,2-difluoro-1-(hydroxymethyl)cyclopropyl)methoxy)pyrido[4′,3′:4,5]thieno[2,3-d]pyrimidin-4-yl)-6-methyl-1,4-oxazepan-6-ol (50 mg, 0.069 mmol) in DCM (1 mL) was added triethylamine (0.038 mL, 0.28 mmol) and methanesulfonic anhydride (18 mg, 0.10 mmol) at 20° C. The reaction was stirred at 20° C. for 10 min. The reaction solution was concentrated in vacuo and purified by preparative TLC (SiO₂, DCM/MeOH=20/1) to give ((1S)-1-(((8-(6-chloro-5-cyclopropyl-1-(tetrahydro-2H-pyran-2-yl)-1H-indazol-4-yl)-4-((S)-6-hydroxy-6-methyl-1,4-oxazepan-4-yl)pyrido[4′,3′:4,5]thieno[2,3-d]pyrimidin-2-yl)oxy)methyl)-2,2-difluorocyclopropyl)methyl methanesulfonate. MS (ESI) [M+H]⁺: m/z 805.

### Step D: ((1S)-1-(((8-(6-chloro-5-cyclopropyl-1H-indazol-4-yl)-4-((S)-6-hydroxy-6-methyl-1,4-oxazepan-4-yl)pyrido[4′,3′:4,5]thieno[2,3-d]pyrimidin-2-yl)oxy)methyl)-2,2-difluorocyclopropyl methyl methanesulfonate

To a stirred solution of ((1S)-1-(((8-(6-chloro-5-cyclopropyl-1-(tetrahydro-2H-pyran-2-yl)-1H-indazol-4-yl)-4-((S)-6-hydroxy-6-methyl-1,4-oxazepan-4-yl)pyrido[4′,3′:4,5]thieno[2,3-d]pyrimidin-2-yl)oxy)methyl)-2,2-difluorocyclopropyl)methyl methanesulfonate (35 mg, 0.043 mmol) in DCM (1 mL) was added TFA (1 mL) at 20° C. The reaction was stirred at 20° C. for 1 h. The reaction was evaporated under reduced pressure to give ((1S)-1-(((8-(6-chloro-5-cyclopropyl-1H-indazol-4-yl)-4-((S)-6-hydroxy-6-methyl-1,4-oxazepan-4-yl)pyrido[4′,3′:4,5]thieno[2,3-d]pyrimidin-2-yl)oxy)methyl)-2,2-difluorocyclopropyl)methyl methanesulfonate. MS (ESI) [M+H]⁺: m/z 721.

### Step E: (6S)-4-(8-(6-chloro-5-cyclopropyl-1H-indazol-4-yl)-2-(((R)-2,2-difluoro-1-((6-fluoro-2-azaspiro[3,3]heptan-2-yl)methyl)cyclopropyl)methoxy)pyrido[4′,3′:4,5]thieno[2,3-d]pyrimidin-4-yl)-6-methyl-1,4-oxazepan-6-ol (Ex-230)

To a solution of ((1S)-1-(((8-(6-chloro-5-cyclopropyl-1H-indazol-4-yl)-4-((S)-6-hydroxy-6-methyl-1,4-oxazepan-4-yl)pyrido[4′,3′:4,5]thieno[2,3-d]pyrimidin-2-yl)oxy)methyl)-2,2-difluorocyclopropyl)methyl methanesulfonate (31 mg, 0.043 mmol) in MeCN (0.5 mL) was added N,N-diisopropylethylamine (0.076 mL, 0.43 mmol) and 6-fluoro-2-azaspiro[3,3]heptane, TFA salt (19 mg, 0.087 mmol) at 20° C. under N₂. The reaction was stirred at 80° C. for 12 h. The reaction solution was purified by preparative reverse-phase HPLC (MeCN/H₂O w/formic acid modifier) to give (6S)-4-(8-(6-chloro-5-cyclopropyl-1H-indazol-4-yl)-2-(((R)-2,2-difluoro-1-((6-fluoro-2-azaspiro[3,3]heptan-2-yl)methyl)cyclopropyl)methoxy)pyrido[4′,3′:4,5]thieno[2,3-d]pyrimidin-4-yl)-6-methyl-1,4-oxazepan-6-ol, formic acid salt (Ex-230). MS (ESI) [M+H]⁺: m/z 740. ¹H NMR (400 MHz, CD₃OD) & 8.79 (dd, J=1.31, 5.60 Hz, 1H), 7.94 (dd, J=1.91, 5.60 Hz, 1H), 7.84 (s, 1H), 7.63 (d, J=8.70 Hz, 1H), 4.90-5.01 (m, 1H), 4.59 (br d, J=11.44 Hz, 1H), 4.46 (br d, J=11.56 Hz, 1H), 4.03-4.22 (m, 3H), 3.89-4.02 (m, 3H), 3.71 (dd, J=8.82, 12.52 Hz, 1H), 3.48-3.61 (m, 5H), 2.94 (br s, 2H), 2.50-2.62 (m, 2H), 2.29 (br s, 2H), 2.00-2.08 (m, 1H), 1.64 (br s, 1H), 1.48-1.58 (m, 1H), 1.11-1.23 (m, 3H), 1.11-1.11 (m, 1H), 0.66-0.84 (m, 1H), 0.38-0.52 (m, 1H), 0.14 (br d, J=5.48 Hz, 2H).

The examples in the table below were synthesized using a similar procedure as described in the synthesis of Ex-230 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Ex. | Material | Structure | Compound Name | Found |
| Ex- 231 | 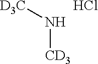 | 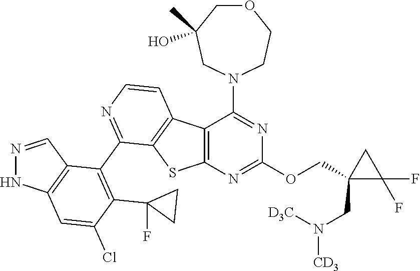 [SMILES: [2H]C([2H])([2H])N(C[C@@]1(COc2nc(N3CCOC[C@@](C)(O)C3)c3c(n2)sc2c(-c4c(C5(F)CC5)c(Cl)cc5[nH]ncc45)nccc23)CC1(F)F)C([2H])([2H])[2H]] | (6S)-4-(2-(((R)-1- ((bis(methy]- d₃)amino)methyl)-2,2- difluorocyclopropyl)methoxy)- 8-(6-chloro-5-(1- fluorocyclopropyl)-1H- indazol-4- yl)pyrido[4′,3′:4,5]thieno[2,3- d]pyrimidin-4-y1)-6-methyl- 1,4-oxazepan-6-ol | 694 |
| Ex- 232 | 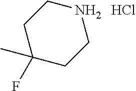 | 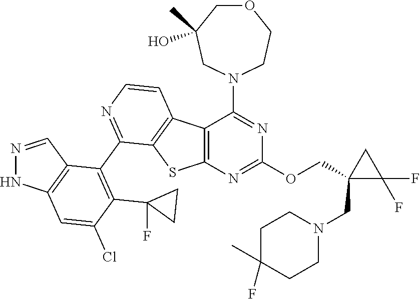 [SMILES: CC1(F)CCN(C[C@@]2(COc3nc(N4CCOC[C@@](C)(O)C4)c4c(n3)sc3c(-c5c(C6(F)CC6)c(Cl)cc6[nH]ncc56)nccc34)CC2(F)F)CC1] | (6S)-4-(8-(6-chloro-5-(1- fluorocyclopropyl)-1H- indazol-4-yl)-2-(((R)-2,2- difluoro-1-((4-fluoro-4- methylpiperidin-1- yl)methyl)cyclopropyl)metho- xy)pyrido[4′,3′:4,5]thieno[2,3- d]pyrimidin-4-yl)-6-methyl- 1,4-oxazepan-6-ol | 760 |
| Ex- 233 | 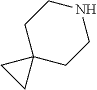 | 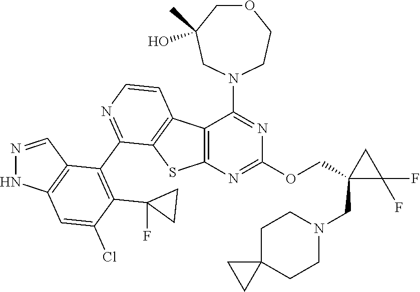 [SMILES: C[C@@]1(O)COCCN(c2nc(OC[C@]3(CN4CCC5(CC4)CC5)CC3(F)F)nc3sc4c(-c5c(C6(F)CC6)c(Cl)cc6[nH]ncc56)nccc4c23)C1] | (6S)-4-(2-(((R)-1-((6- azaspiro[2.5]octan-6- yl)methyl)-2,2- difluorocyclopropyl)methoxy)- 8-(6-chloro-5-(1- fluorocyclopropyl)-1H- indazol-4- yl)pyrido[4′,3′:4,5]thieno[2,3- d]pyrimidin-4-yl)-6-methyl- 1,4-oxazepan-6-ol | 754 |
| Ex- 234 | 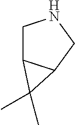 | 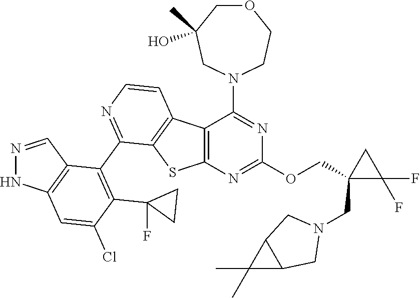 [SMILES: CC1(C)C2CN(C[C@@]3(COc4nc(N5CCOC[C@@](C)(O)C5)c5c(n4)sc4c(-c6c(C7(F)CC7)c(Cl)cc7[nH]ncc67)nccc45)CC3(F)F)CC21] | (6S)-4-(8-(6-chloro-5-(1- fluorocyclopropyl)-1H- indazol-4-yl)-2-(((1R)-1- ((6,6-dimethyl-3- azabicyclo[3.1.0]hexan-3- yl)methyl)-2,2- difluorocyclopropyl)methoxy) pyrido[4′,3′:4,5]thieno[2,3- d]pyrimidin-4-yl)-6-methyl- 1,4-oxazepan-6-ol | 754 |
| Ex- 235 | 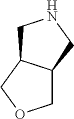 | 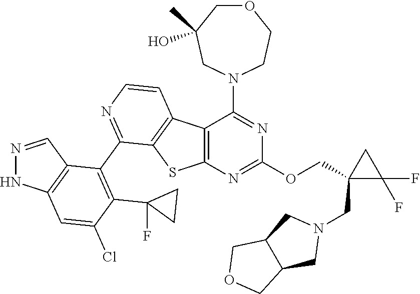 [SMILES: C[C@@]1(O)COCCN(c2nc(OC[C@]3(CN4C[C@H]5COC[C@H]5C4)CC3(F)F)nc3sc4c(-c5c(C6(F)CC6)c(Cl)cc6[nH]ncc56)nccc4c23)C1] | (6S)-4-(8-(6-chloro-5-(1- fluorocyclopropyl)-1H- indazol-4-yl)-2-(((R)-2,2- difluoro-1-(((3aR,6aS)- tetrahydro-1H-furo[3,4- c]pyrrol-5(3H)- yl)methyl)cyclopropyl)metho- xy)pyrido[4′,3′:4,5]thieno[2,3- d]pyrimidin-4-yl)-6-methyl- 1,4-oxazepan-6-ol | 756 |
| Ex- 236 | 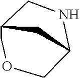 | 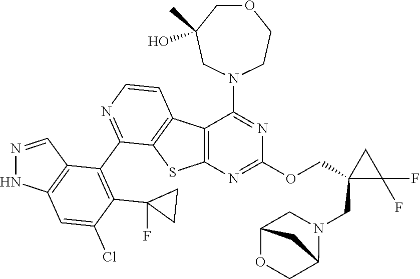 [SMILES: C[C@@]1(O)COCCN(c2nc(OC[C@]3(CN4C[C@@H]5C[C@H]4CO5)CC3(F)F)nc3sc4c(-c5c(C6(F)CC6)c(Cl)cc6[nH]ncc56)nccc4c23)C1] | (6S)-4-(2-(((R)-1-(((15,4S)-2- oxa-5- azabicyclo[2.2.1]heptan-5- yl)methyl)-2,2- difluorocyclopropyl)methoxy)- 8-(6-chloro-5-(1- fluorocyclopropyl)-1H- indazol-4- yl)pyrido[4′,3′:4,5]thieno[2,3- d]pyrimidin-4-yl)-6-methyl- 1,4-oxazepan-6-ol | 742 |
| Ex- 237 | 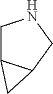 | 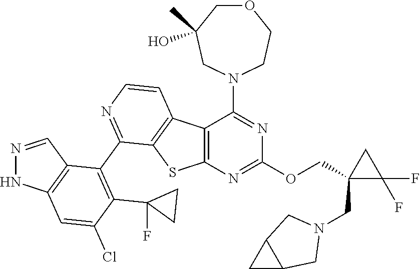 [SMILES: C[C@@]1(O)COCCN(c2nc(OC[C@]3(CN4CC5CC5C4)CC3(F)F)nc3sc4c(-c5c(C6(F)CC6)c(Cl)cc6[nH]ncc56)nccc4c23)C1] | (6S)-4-(2-(((1R)-1-((3- azabicyclo[3.1.0]hexan-3- yl)methyl)-2,2- difluorocyclopropyl)methoxy)- 8-(6-chloro-5-(1- fluorocyclopropyl)-1H- indazol-4- yl)pyrido[4′,3′:4,5]thieno[2,3- d]pyrimidin-4-yl)-6-methyl- 1,4-oxazepan-6-ol | 726 |
| Ex- 238 | 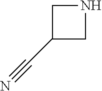 | 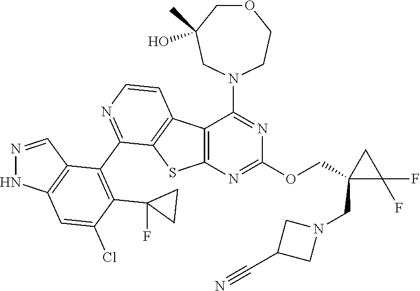 [SMILES: C[C@@]1(O)COCCN(c2nc(OC[C@]3(CN4CC(C#N)C4)CC3(F)F)nc3sc4c(-c5c(C6(F)CC6)c(Cl)cc6[nH]ncc56)nccc4c23)C1] | 1-(((IR)-1-(((8-(6-chloro-5- (1-fluorocyclopropyl)-1H- indazol-4-yl)-4-((S)-6- hydroxy-6-methyl-1,4- oxazepan-4- yl)pyrido[4′,3′:4,5]thieno[2,3- d]pyrimidin-2- yl)oxy)methyl)-2,2- difluorocyclopropyl)methyl]a- zetidine-3-carbonitrile | 725 |
| Ex- 239 | 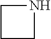 | 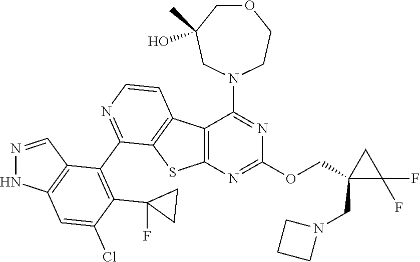 [SMILES: C[C@@]1(O)COCCN(c2nc(OC[C@]3(CN4CCC4)CC3(F)F)nc3sc4c(-c5c(C6(F)CC6)c(Cl)cc6[nH]ncc56)nccc4c23)C1] | (6S)-4-(2-(((R)-1-(azetidin-1- ylmethyl)-2,2- difluorocyclopropyl)methoxy)- 8-(6-chloro-5-(1- fluorocyclopropyl)-1H- indazol-4- yl)pyrido[4′,3:′4,5]thieno[2,3- d]pyrimidin-4-yl)-6-methyl- 1,4-oxazepan-6-ol | 700 |
| 240 | 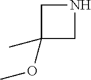 | 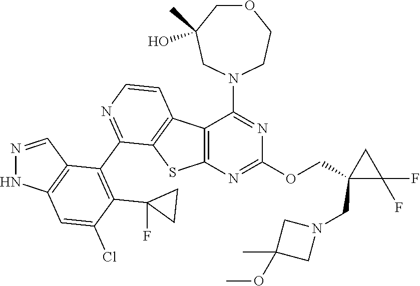 [SMILES: COC1(C)CN(C[C@@]2(COc3nc(N4CCOC[C@@](C)(O)C4)c4c(n3)sc3c(-c5c(C6(F)CC6)c(Cl)cc6[nH]ncc56)nccc34)CC2(F)F)C1] | (6S)-4-(8-(6-chloro-5-(1- fluorocyclopropyl)-1H- indazol-4-yl)-2-(((R)-2,2- difluoro- 1-((3-methoxy-3- methylazetidin-1- yl)methyl)cyclopropyl)metho- xy)pyrido[4′,3′:4,5]thieno[2,3- d]pyrimidin-4-yl)-6-methyl- 1,4-oxazepan-6-ol | 744 |
| Ex- 241 | 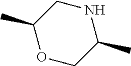 | 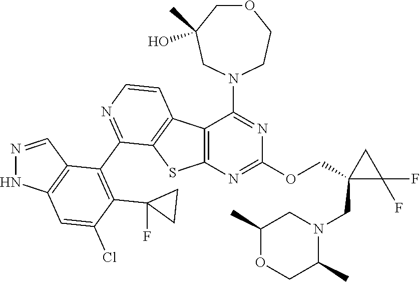 [SMILES: C[C@H]1CN(C[C@@]2(COc3nc(N4CCOC[C@@](C)(O)C4)c4c(n3)sc3c(-c5c(C6(F)CC6)c(Cl)cc6[nH]ncc56)nccc34)CC2(F)F)[C@@H](C)CO1] | (6S)-4-(8-(6-chloro-5-(1- fluorocyclopropyl)-1H- indazol-4-yl)-2-(((R)-1- (((2S,5S)-2,5- dimethylmorpholino)methyl)- 2,2- difluorocyclopropyl)methoxy) pyrido[4′,3′:4,5]thieno[2,3- d]pyrimidin-4-yl)-6-methyl- 1,4-oxazepan-6-ol | 758 |
| Ex- 242 | 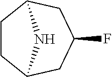 | 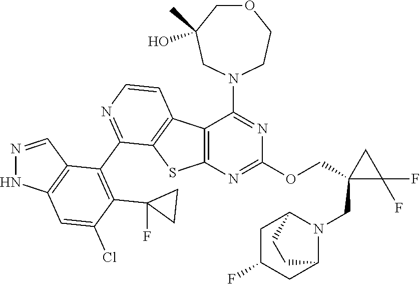 [SMILES: C[C@@]1(O)COCCN(c2nc(OC[C@]3(CN4[C@@H]5CC[C@H]4C[C@@H](F)C5)CC3(F)F)nc3sc4c(-c5c(C6(F)CC6)c(Cl)cc6[nH]ncc56)nccc4c23)C1] | (6S)-4-(8-(6-chloro-5-(1- fluorocyclopropyl)-1H- indazol-4-yl)-2-(((R)-2,2- difluoro-1-(((1R,3R,5S)-3- fluoro-8- azabicyclo[3.2.1]octan-8- yl)methyl)cyclopropyl)metho- xy)pyrido[4′,3′:4,5] thieno[2,3- d]pyrimidin-4-yl)-6-methyl- 1,4-oxazepan-6-ol | 772 |
| Ex- 243 | 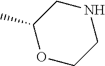 | 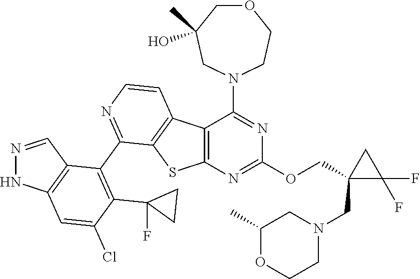 [SMILES: C[C@@H]1CN(C[C@@]2(COc3nc(N4CCOC[C@@](C)(O)C4)c4c(n3)sc3c(-c5c(C6(F)CC6)c(Cl)cc6[nH]ncc56)nccc34)CC2(F)F)CCO1] | (6S)-4-(8-(6-chloro-5-(1- indazol-4-yl)-2-(((R)-2,2- difluoro-1-(((R)-2- methylmorpholino)methyl)cy- clopropyl)methoxy)pyrido[4′, 3′:4,5] thieno[2,3- d]pyrimidin-4-yl)-6-methyl- 1,4-oxazepan-6-ol | 744 |
| Ex- 244 | 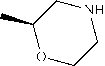 | 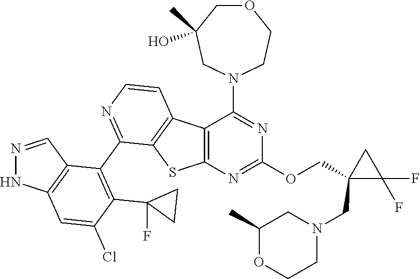 [SMILES: C[C@H]1CN(C[C@@]2(COc3nc(N4CCOC[C@@](C)(O)C4)c4c(n3)sc3c(-c5c(C6(F)CC6)c(Cl)cc6[nH]ncc56)nccc34)CC2(F)F)CCO1] | (6S)-4-(8-(6-chloro-5-(1- fluorocyclopropyl)-1H- indazol-4-yl)-2-(((R)-2,2- difluoro-1-(((S)-2- methylmorpholino)methyl)cy- clopropyl)methoxy)pyrido[4′, 3′:4,5]thieno[2,3- d]pyrimidin-4-yl)-6-methyl- 1,4-oxazepan-6-ol | 744 |
| Ex- 245 | 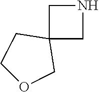 | 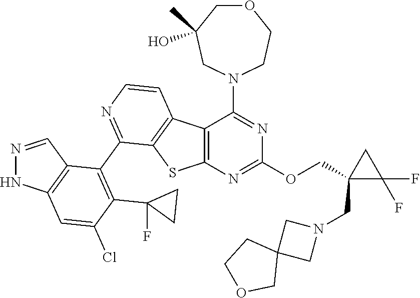 [SMILES: C[C@@]1(O)COCCN(c2nc(OC[C@]3(CN4CC5(CCOC5)C4)CC3(F)F)nc3sc4c(-c5c(C6(F)CC6)c(Cl)cc6[nH]ncc56)nccc4c23)C1] | (6S)-4-(2-(((R)-1-((6-oxa-2- azaspiro[3.4]octan-2- yl)methyl)-2,2- difluorocyclopropyl)methoxy)- 8-(6-chloro-5-(1- fluorocyclopropyl)-1H- indazol-4- yl)pyrido[4′,3′:4,5]thieno[2,3- d)pyrimidin-4-yl)-6-methyl- 1,4-oxazepan-6-ol | 756 |
| Ex- 246 | 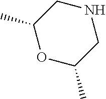 | 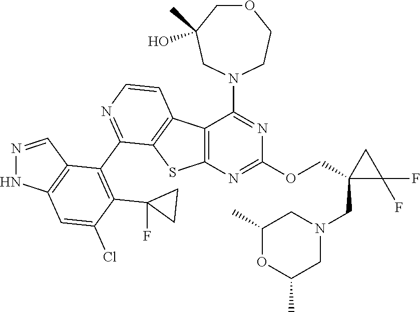 [SMILES: C[C@@H]1CN(C[C@@]2(COc3nc(N4CCOC[C@@](C)(O)C4)c4c(n3)sc3c(-c5c(C6(F)CC6)c(Cl)cc6[nH]ncc56)nccc34)CC2(F)F)C[C@H](C)O1] | (6S)-4-(8-(6-chloro-5-(1- fluorocyclopropyl)-1H- indazol-4-yl)-2-(((R)-1- (((2R,6S)-2,6- dimethylmorpholino)methyl)- 2,2- difluorocyclopropyl)methoxy) pyrido[4′,3′:4,5]thieno[2,3- d]pyrimidin-4-yl)-6-methyl- 1,4-oxazepan-6-ol | 758 |
| Ex- 247 | 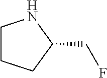 | 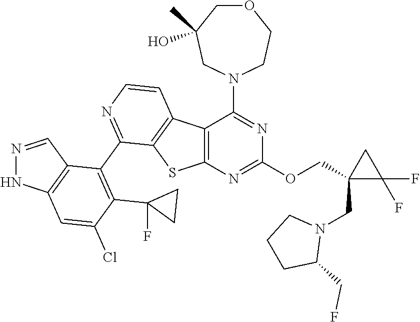 [SMILES: C[C@@]1(O)COCCN(c2nc(OC[C@]3(CN4CCC[C@H]4CF)CC3(F)F)nc3sc4c(-c5c(C6(F)CC6)c(Cl)cc6[nH]ncc56)nccc4c23)C1] | (6S)-4-(8-(6-chloro-5-(1- fluorocyclopropyl)-1H- indazol-4-yl)-2-(((R)-2,2- difluoro-1-(((S)-2- (fluoromethyl)pyrrolidin-1- yl)methyl)cyclopropyl)metho- xy)pyrido[4′,3′:4,5]thieno[2,3- d]pyrimidin-4-yl)-6-methyl- 1,4-oxazepan-6-ol | 746 |
| Ex- 248 | 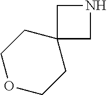 | 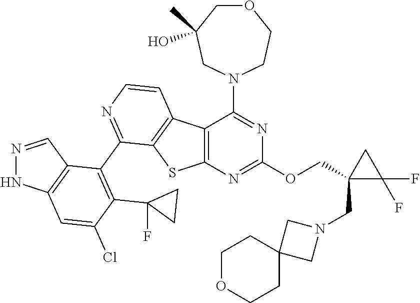 [SMILES: C[C@@]1(O)COCCN(c2nc(OC[C@]3(CN4CC5(CCOCC5)C4)CC3(F)F)nc3sc4c(-c5c(C6(F)CC6)c(Cl)cc6[nH]ncc56)nccc4c23)C1] | (6S)-4-(2-(((R)-1-((7-oxa-2- azaspiro[3.5]nonan-2- yl)methyl)-2,2- difluorocyclopropyl)methoxy)- 8-(6-chloro-5-(1- fluorocyclopropyl)-1H- indazol-4- yl)pyrido[4′,3′:4,5]thieno[2,3- d]pyrimidin-4-y1)-6-methyl- 1,4-oxazepan-6-ol | 770 |
| Ex- 249 | 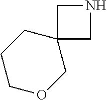 | 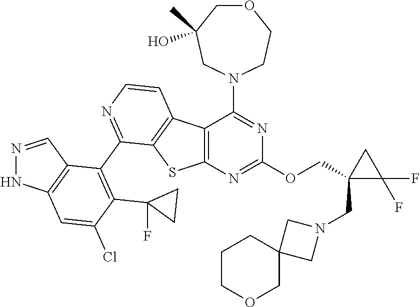 [SMILES: C[C@@]1(O)COCCN(c2nc(OC[C@]3(CN4CC5(CCCOC5)C4)CC3(F)F)nc3sc4c(-c5c(C6(F)CC6)c(Cl)cc6[nH]ncc56)nccc4c23)C1] | (6S)-4-(2-(((R)-1-((6-oxa-2- azaspiro[3.5]nonan-2- yl)methyl)-2,2- difluorocyclopropyl)methoxy)- 8-(6-chloro-5-(1- fluorocyclopropyl)-1H- indazol-4- yl)pyrido[4′,3′:4,5]thieno[2,3- d]pyrimidin-4-yl)-6-methyl- 1,4-oxazepan-6-ol | 770 |
| Ex- 250 | 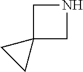 | 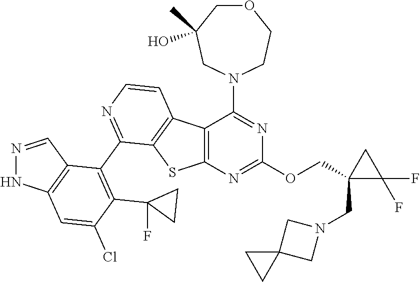 [SMILES: C[C@@]1(O)COCCN(c2nc(OC[C@]3(CN4CC5(CC5)C4)CC3(F)F)nc3sc4c(-c5c(C6(F)CC6)c(Cl)cc6[nH]ncc56)nccc4c23)C1] | (6S)-4-(2-(((R)-1-((5- azaspiro[2.3]hexan-5- yl)methyl)-2,2- difluorocyclopropyl)methoxy)- 8-(6-chloro-5-(1- fluorocyclopropyl)-1H- indazol-4- yl)pyrido[4′,3′:4,5]thieno[2,3- d]pyrimidin-4-yl)-6-methyl- 1,4-oxazepan-6-ol | 726 |
| Ex- 251 | 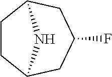 | 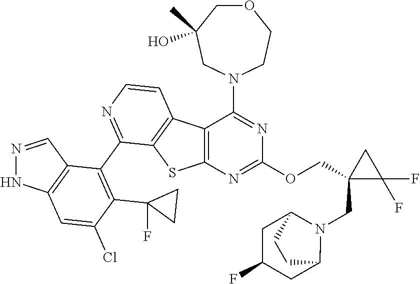 [SMILES: C[C@@]1(O)COCCN(c2nc(OC[C@]3(CN4[C@@H]5CC[C@H]4C[C@H](F)C5)CC3(F)F)nc3sc4c(-c5c(C6(F)CC6)c(Cl)cc6[nH]ncc56)nccc4c23)C1] | (6S)-4-(8-(6-chloro-5-(1- fluorocyclopropyl)-1H- indazol-4-yl)-2-(((R)-2,2- difluoro-1-(((1R,3S,5S)-3- fluoro-8- azabicyclo[3.2.1]octan-8- yl)methyl)cyclopropyl)metho- xy)pyrido[4′,3′:4,5]thieno[2,3- d]pyrimidin-4-yl)-6-methyl- 1,4-oxazepan-6-ol | 772 |
| Ex- 252 | 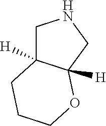 | 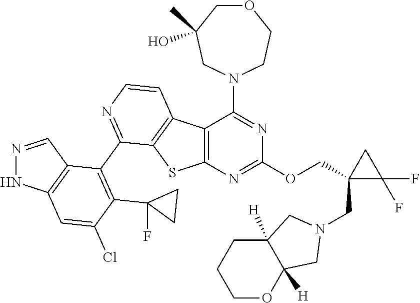 [SMILES: C[C@@]1(O)COCCN(c2nc(OC[C@]3(CN4C[C@@H]5CCCO[C@H]5C4)CC3(F)F)nc3sc4c(-c5c(C6(F)CC6)c(Cl)cc6[nH]ncc56)nccc4c23)C1] | (6S)-4-(8-(6-chloro-5-(1- fluorocyclopropyl)-1H- indazol-4-yl)-2-(((R)-2,2- difluoro-1-(((4aS,7aR)- hexahydropyrano[2,3- c]pyrrol-6(2H)- yl)methyl)cyclopropyl)methoxy) pyrido[4′,3′:4,5]thieno[2,3- d]pyrimidin-4-yl)-6-methyl- 1,4-oxazepan-6-ol | 770 |
| Ex- 253 | 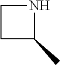 | 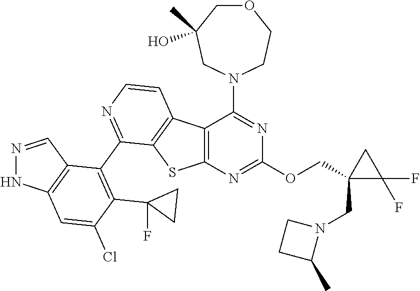 [SMILES: C[C@H]1CCN1C[C@@]1(COc2nc(N3CCOC[C@@](C)(O)C3)c3c(n2)sc2c(-c4c(C5(F)CC5)c(Cl)cc5[nH]ncc45)nccc23)CC1(F)F] | (6S)-4-(8-(6-chloro-5-(1- fluorocyclopropyl)-1H- indazol-4-yl)-2-(((R)-2,2- difluoro-1-(((S)-2- methylazetidin-1- yl)methyl)cyclopropyl)metho- xy)pyrido[4′,3′:4,5]thieno[2,3- d]pyrimidin-4-yl)-6-methyl- 1,4-oxazepan-6-ol | 714 |
| Ex- 254 | 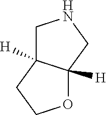 | 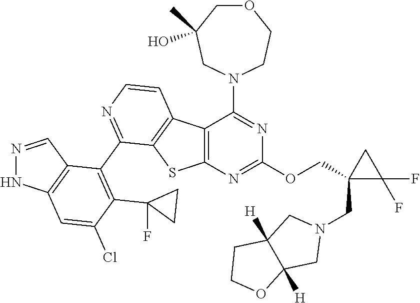 [SMILES: C[C@@]1(O)COCCN(c2nc(OC[C@]3(CN4C[C@H]5CCO[C@H]5C4)CC3(F)F)nc3sc4c(-c5c(C6(F)CC6)c(Cl)cc6[nH]ncc56)nccc4c23)C1] | (6S)-4-(8-(6-chloro-5-(1- fluorocyclopropyl)-1H- indazol-4-yl)-2-(((R)-2,2- difluoro-1-(((3aR,6aR)- hexahydro-5H-furo[2,3- c]pyrrol-5- yl)methyl)cyclopropyl)metho- xy)pyrido[4′,3′:4,5]thieno[2,3- d]pyrimidin-4-yl)-6-methyl- 1,4-oxazepan-6-ol | 756 |
| Ex- 255 | 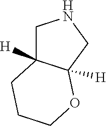 | 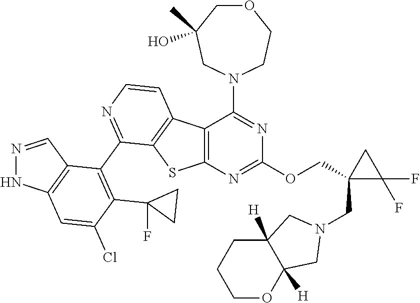 [SMILES: C[C@@]1(O)COCCN(c2nc(OC[C@]3(CN4C[C@H]5CCCO[C@H]5C4)CC3(F)F)nc3sc4c(-c5c(C6(F)CC6)c(Cl)cc6[nH]ncc56)nccc4c23)C1] | (6S)-4-(8-(6-chloro-5-(1- fluorocyclopropyl)-1H- indazol-4-yl)-2-(((R)-2,2- difluoro-1-(((4aR,7aR)- hexahydropyrano[2,3- c]pyrrol-6(2H)- yl)methyl)cyclopropyl)metho- xy)pyrido[4′,3′:4,5]thieno[2,3- d]pyrimidin-4-yl)-6-methyl- 1,4-oxazepan-6-o1 | 770 |
| Ex- 256 | 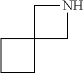 | 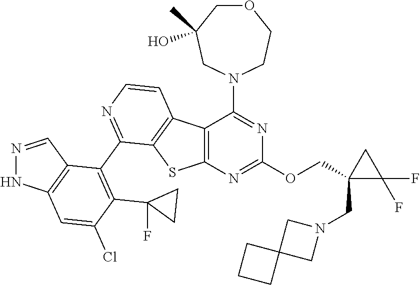 [SMILES: C[C@@]1(O)COCCN(c2nc(OC[C@]3(CN4CC5(CCC5)C4)CC3(F)F)nc3sc4c(-c5c(C6(F)CC6)c(Cl)cc6[nH]ncc56)nccc4c23)C1] | (6S)-4-(2-(((R)-1-((2- azaspiro[3.3]heptan-2- yl)methyl)-2,2- difluorocyclopropyl)methoxy)- 8-(6-chloro-5-(1- fluorocyclopropyl)-1H- indazol-4- yl)pyrido[4′,3′:4,5]thieno[2,3- d]pyrimidin-4-yl)-6-methyl- 1,4-oxazepam-6-ol | 740 |
| Ex- 257 | 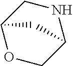 | 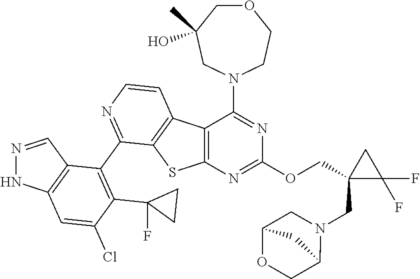 [SMILES: C[C@@]1(O)COCCN(c2nc(OC[C@]3(CN4C[C@H]5C[C@@H]4CO5)CC3(F)F)nc3sc4c(-c5c(C6(F)CC6)c(Cl)cc6[nH]ncc56)nccc4c23)C1] | (6S)-4-(2-(((R)-1-(((1R,4R)- 2-oxa-5- azabicyclo[2.2.1]heptan-5- yl)methyl)-2,2- difluorocyclopropyl)methoxy)- 8-(6-chloro-5-(1- fluorocyclopropyl)-1H- indazol-4- yl)pyrido[4′,3′:4,5]thieno[2,3- d]pyrimidin-4-yl)-6-methyl- 1,4-oxazepan-6-ol | 742 |
| Ex- 258 | 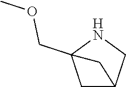 | 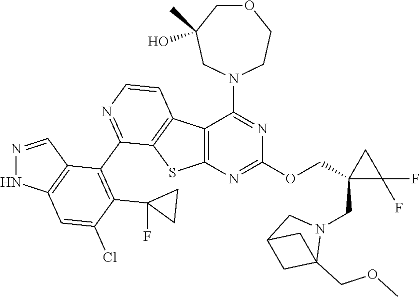 [SMILES: COCC12CC(CN1C[C@@]1(COc3nc(N4CCOC[C@@](C)(O)C4)c4c(n3)sc3c(-c5c(C6(F)CC6)c(Cl)cc6[nH]ncc56)nccc34)CC1(F)F)C2] | (6S)-4-(8-(6-chloro-5-(1- fluorocyclopropyl)-1H- indazol-4-yl)-2-(((R)-2,2- difluoro-1-((1- (methoxymethyl)-2- azabicyclo[2.1.1]hexan-2- yl)methyl)cyclopropyl)metho- xy)pyrido[4′,3′:4,5]thieno[2,3- d]pyrimidin-4-yl)-6-methyl- 1,4-oxazepan-6-ol | 770 |
| Ex- 259 | 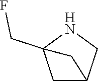 | 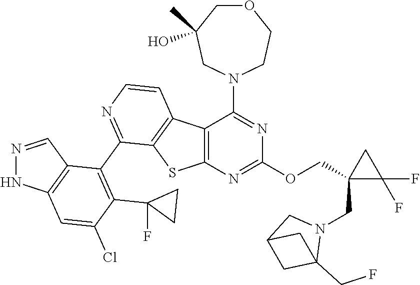 [SMILES: C[C@@]1(O)COCCN(c2nc(OC[C@]3(CN4CC5CC4(CF)C5)CC3(F)F)nc3sc4c(-c5c(C6(F)CC6)c(Cl)cc6[nH]ncc56)nccc4c23)C1] | (6S)-4-(8-(6-chloro-5-(1- fluorocyclopropyl)-1H- indazol-4-yl)-2-(((R)-2,2- difluoro-1-((1- (fluoromethyl)-2- azabicyclo[2.1.1]hexan-2- yl)methyl)cyclopropyl)metho- xy)pyrido[4′,3′:4,5]thieno[2,3- d]pyrimidin-4-yl)-6-methyl- 1,4-oxazepan-6-ol | 758 |
| Ex- 260 | 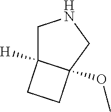 | 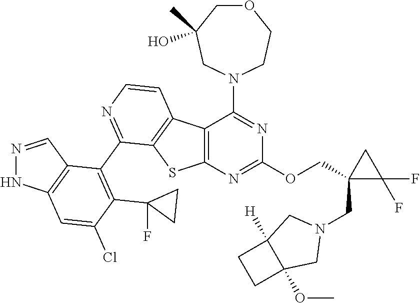 [SMILES: CO[C@]12CC[C@H]1CN(C[C@@]1(COc3nc(N4CCOC[C@@](C)(O)C4)c4c(n3)sc3c(-c5c(C6(F)CC6)c(Cl)cc6[nH]ncc56)nccc34)CC1(F)F)C2] | (6S)-4-(8-(6-chloro-5-(1- fluorocyclopropyl)-1H- indazol-4-yl)-2-(((R)-2,2- difluoro-1-(((1R,5S)-1- methoxy-3- azabicyclo[3.2.0]heptan-3- yl)methyl)cyclopropyl)metho- xy)pyrido[4′,3′:4,5]thieno[2,3- d]pyrimidin-4-yl)-6-methyl- 1,4-oxazepan-6-ol | 770 |
| Ex- 261 | 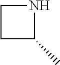 | 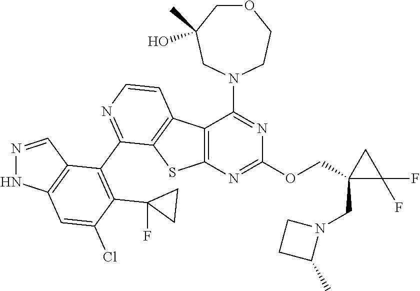 [SMILES: C[C@@H]1CCN1C[C@@]1(COc2nc(N3CCOC[C@@](C)(O)C3)c3c(n2)sc2c(-c4c(C5(F)CC5)c(Cl)cc5[nH]ncc45)nccc23)CC1(F)F] | (6S)-4-(8-(6-chloro-5-(1- fluorocyclopropyl)-1H- indazol-4-yl)-2-(((R)-2,2- difluoro-1-(((R)-2- methylazetidin-1- yl)methyl)cyclopropyl)metho- xy)pyrido[4′,3′:4,5]thieno[2,3- d]pyrimidin-4-yl)-6-methyl- 1,4-oxazepan-6-ol | 714 |
| Ex- 262 | 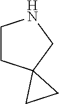 | 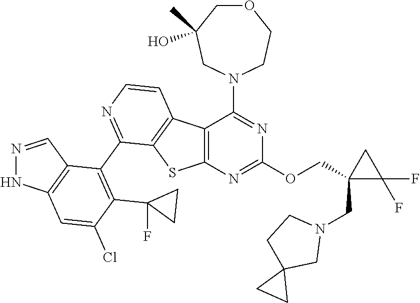 [SMILES: C[C@@]1(O)COCCN(c2nc(OC[C@]3(CN4CCC5(CC5)C4)CC3(F)F)nc3sc4c(-c5c(C6(F)CC6)c(Cl)cc6[nH]ncc56)nccc4c23)C1] | (6S)-4-(2-(((R)-1-((5- azaspiro[2.4]heptan-5- yl)methyl)-2,2- difluorocyclopropyl)methoxy)- 8-(6-chloro-5-(1- fluorocyclopropyl)-1H- indazol-4- yl)pyrido[4′,3′:4,5]thieno[2,3- d]pyrimidin-4-yl)-6-methyl- 1,4-oxazepan-6-ol | 740 |
| Ex- 263 | 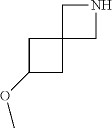 | 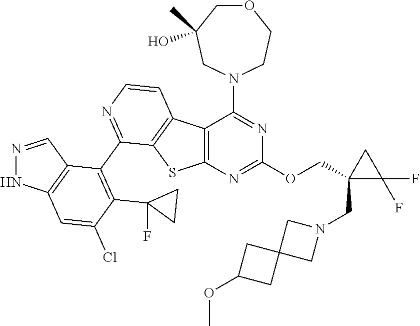 [SMILES: COC1CC2(C1)CN(C[C@@]1(COc3nc(N4CCOC[C@@](C)(O)C4)c4c(n3)sc3c(-c5c(C6(F)CC6)c(Cl)cc6[nH]ncc56)nccc34)CC1(F)F)C2] | (6S)-4-(8-(6-chloro-5-(1- fluorocyclopropyl)-1H- indazol-4-yl)-2-(((R)-2,2- difluoro-1-((6-methoxy-2- azaspiro[3.3]heptan-2- yl)methyl)cyclopropyl)metho- xy)pyrido[4′,3′:4,5]thieno[2,3- d]pyrimidin-4-yl)-6-methyl- 1,4-oxazepan-6-ol | 770 |
| Ex- 264 | 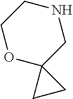 | 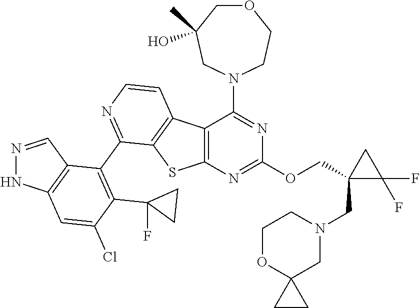 [SMILES: C[C@@]1(O)COCCN(c2nc(OC[C@]3(CN4CCOC5(CC5)C4)CC3(F)F)nc3sc4c(-c5c(C6(F)CC6)c(Cl)cc6[nH]ncc56)nccc4c23)C1] | (6S)-4-(2-(((R)-1-((4-oxa-7- azaspiro[2.5]octan-7- yl)methyl)-2,2- difluorocyclopropyl)methoxy)- 8-(6-chloro-5-(1- fluorocyclopropyl)-1H- indazol-4- yl)pyrido[4′,3′:4,5]thieno[2,3- d]pyrimidin-4-yl)-6-methyl- 1,4-oxazepan-6-ol | 756 |
| Ex- 265 | 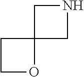 | 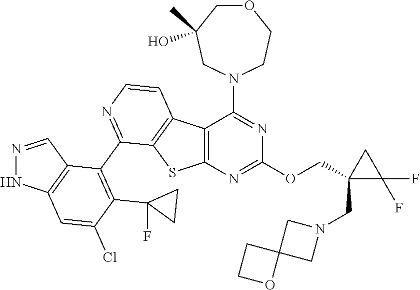 [SMILES: C[C@@]1(O)COCCN(c2nc(OC[C@]3(CN4CC5(CCO5)C4)CC3(F)F)nc3sc4c(-c5c(C6(F)CC6)c(Cl)cc6[nH]ncc56)nccc4c23)C1] | (6S)-4-(2-(((R)-1-((1-oxa-6- azaspiro[3.3]heptan-6- yl)methyl)-2,2- difluorocyclopropyl)methoxy)- 8-(6-chloro-5-(1- fluorocyclopropyl)-1H- indazol-4- yl)pyrido[4′,3′:4,5]thieno[2,3- d]pyrimidin-4-yl)-6-methyl- 1,4-oxazepan-6-ol | 742 |
| Ex- 266 | 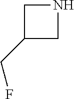 | 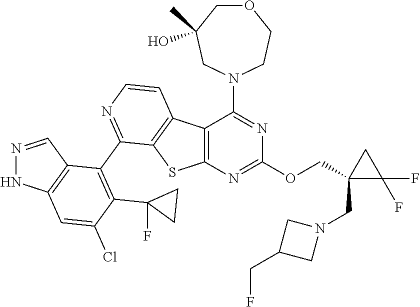 [SMILES: C[C@@]1(O)COCCN(c2nc(OC[C@]3(CN4CC(CF)C4)CC3(F)F)nc3sc4c(-c5c(C6(F)CC6)c(Cl)cc6[nH]ncc56)nccc4c23)C1] | (6S)-4-(8-(6-chloro-5-(1- fluorocyclopropyl)-1H- indazol-4-yl)-2-(((R)-2,2- difluoro-1-((3- (fluoromethyl)azetidin-1- yl)methyl)cyclopropyl)metho- xy)pyrido[4′,3′:4,5]thieno[2,3- d]pyrimidin-4-yl)-6-methyl- 1,4-oxazepan-6-ol | 732 |

### Example 267: (6S)-4-(8-(6-chloro-5-(1-fluorocyclopropyl)-1H-indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-267)

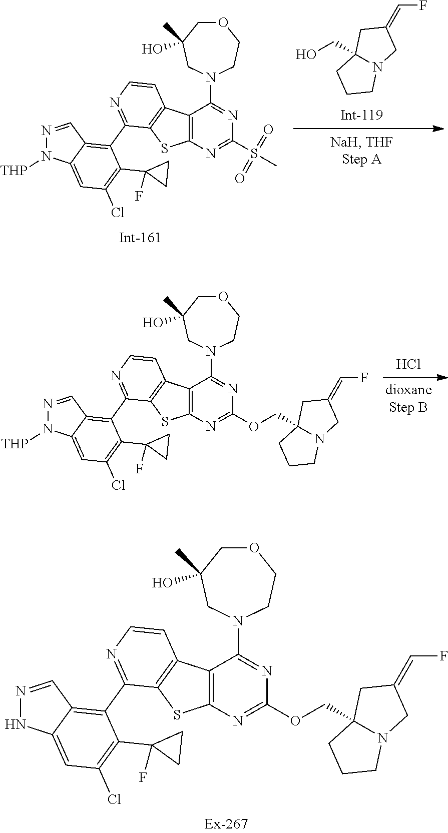

### Step A: (6S)-4-(8-(6-chloro-5-(1-fluorocyclopropyl)-1-(tetrahydro-2H-pyran-2-yl)-1H-indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol

To a solution of (6S)-4-(8-(6-chloro-5-(1-fluorocyclopropyl)-1-(tetrahydro-2H-pyran-2-yl)-1H-indazol-4-yl)-2-(methylsulfonyl)pyrido[4′,3′:4,5]thieno[2,3-d]pyrimidin-4-yl)-6-methyl-1,4-oxazepan-6-ol (Int-161) (800 mg, 1.16 mmol) and (S,Z)-(2-(fluoromethylene)tetrahydro-1H-pyrrolizin-7a(5H)-yl) methanol (Int-119) (239 mg, 1.40 mmol) in THF (10 mL) was added NaH (279 mg, 6.98 mmol, 60% in mineral oil) at 25° C., the reaction mixture was stirred at 25° C. for 1 h under N₂. The reaction mixture was quenched with sat. aq. NH₄Cl (20 mL) and extracted with EtOAc (3×20 mL). The organic layer was dried over sodium sulfate, filtered and concentrated in vacuo. The crude was purified by flash silica gel chromatography (0 to 12% (3:1 EtOAc:EtOH) in petroleum ether) to give (6S)-4-(8-(6-chloro-5-(1-fluorocyclopropyl)-1-(tetrahydro-2H-pyran-2-yl)-1H-indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol. MS (ESI) [M+H]⁺: m/z 778.

### Step B: (6S)-4-(8-(6-chloro-5-(1-fluorocyclopropyl)-1H-indazol-4-yl)-2-(((S,Z)-2-(fluoromethylene)tetrahydro-1H-pyrrolizin-7a (5H)-yl)methoxy)pyrido[4′,3′:4,5]thieno[2,3-d]pyrimidin-4-yl)-6-methyl-1,4-oxazepam-6-ol (Ex-267)

To (6S)-4-(8-(6-chloro-5-(1-fluorocyclopropyl)-1-(tetrahydro-2H-pyran-2-yl)-1H-indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (560 mg, 0.720 mmol) was added HCl (10 mL, 40 mmol, 4.0 M in dioxane) at 25° C. The reaction mixture was stirred for 1 h under N₂. Most of the solvent was removed by nitrogen flow, basified with sat aq. NaHCO₃ (5 mL), and extracted with EtOAc (3×10 mL). The combined organic layers were dried over sodium sulfate, filtered and concentrated in vacuo. The crude product was purified by preparative reverse-phase HPLC (MeCN/H₂O w/NH₃—NH₄HCO₃ modifier) to give (6S)-4-(8-(6-chloro-5-(1-fluorocyclopropyl)-1H-indazol-4-yl)-2-(((S,Z)-2-(fluoromethylene)tetrahydro-1H-pyrrolizin-7a(5H)-yl)methoxy)pyrido[4′,3′:4,5]thieno[2,3-d]pyrimidin-4-yl)-6-methyl-1,4-oxazepan-6-ol (Ex-267). MS (ESI) [M+H]⁺: m/z 694. ¹H NMR (400 MHz, CD₃OD) δ 8.77 (d, J=5.6 Hz, 1H), 7.99-7.82 (m, 2H), 7.62 (s, 1H), 6.78-6.46 (m, 1H), 4.31 (dd, J=4.2, 10.5 Hz, 1H), 4.22-3.76 (m, 8H), 3.69 (dd, J=6.3, 12.5 Hz, 1H), 3.54-3.39 (m, 2H), 3.18-3.06 (m, 1H), 2.74-2.61 (m, 2H), 2.41 (br d, J=15.5 Hz, 1H), 2.13-2.02 (m, 1H), 2.01-1.76 (m, 3H), 1.25-1.13 (m, 3H), 0.99 (dq, J=6.3, 12.0 Hz, 2H), 0.75-0.62 (m, 1H), 0.55-0.32 (m, 1H).

The examples in the table below were synthesized using a similar procedure as described in the synthesis of Ex-267 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | | [M + |
|---|---|---|---|---|
| | Starting | | | H]⁺ |
| Ex | Material | Structure | Compound Name | Found |
| Ex- 268 | 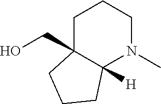 Int-126 | 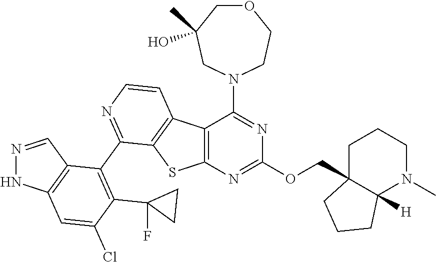 [SMILES: CN1CCC[C@@]2(COc3nc(N4CCOC[C@@](C)(O)C4)c4c(n3)sc3c(-c5c(C6(F)CC6)c(Cl)cc6[nH]ncc56)nccc34)CCC[C@@H]12] | (6S)-4-(8-(6-chloro-5-(1- fluorocyclopropyl)-1H- indazol-4-yl)-2-(((4aS,7aR)- 1-methyloctahydro-4aH- cyclopenta[b]pyridin-4a- yl)methoxy)pyri- do[4′,3′:4,5]thi- eno[2,3-d]pyrimidin-4-yl)- 6-methyl-1,4-oxazepan-6- ol | 692 |
| Ex- 269 | 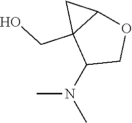 Int-127 | 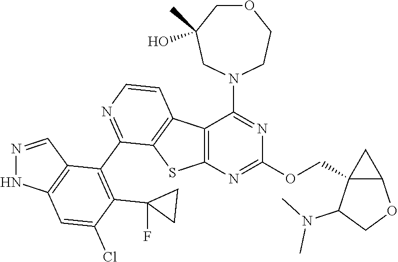 [SMILES: CN(C)C1COC2C[C@@]21COc1nc(N2CCOC[C@@](C)(O)C2)c2c(n1)sc1c(-c3c(C4(F)CC4)c(Cl)cc4[nH]ncc34)nccc12] | (6S)-4-(8-(6-chloro-5-(1- fluorocyclopropyl)-1H- indazol-4-yl)-2-((4- (dimethylamino)-2- oxabicyclo[3.1.0]hexan-5- yl)methoxy)pyri- do[4′,3′:4,5]thi- eno[2,3-d]pyrimidin-4-yl)- 6-methyl-1,4-oxazepan-6- ol | 680 |
| Ex- 270 | 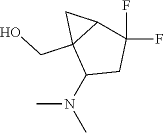 Int-129 | 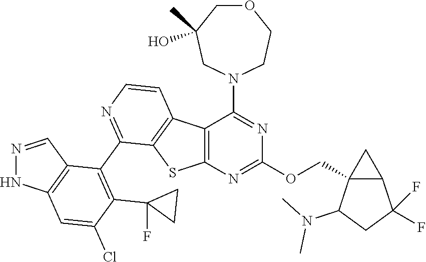 [SMILES: CN(C)C1CC(F)(F)C2C[C@]12COc1nc(N2CCOC[C@@](C)(O)C2)c2c(n1)sc1c(-c3c(C4(F)CC4)c(Cl)cc4[nH]ncc34)nccc12] | (6S)-4-(8-(6-chloro-5-(1- fluorocyclopropyl)-1H- indazol-4-yl)-2-((2- (dimethylamino)-4,4- difluorobicy- clo[3.1.0]hexan- 1-yl)methoxy)pyri- do[4′,3′:4,5]thi- eno[2,3-d]pyrimidin-4-yl)- 6-methyl-1,4-oxazepan-6- ol | 714 |
| Ex- 271 | 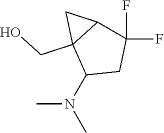 Int-130 | 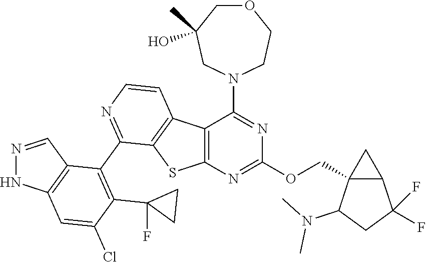 [SMILES: CN(C)C1CC(F)(F)C2C[C@]12COc1nc(N2CCOC[C@@](C)(O)C2)c2c(n1)sc1c(-c3c(C4(F)CC4)c(Cl)cc4[nH]ncc34)nccc12] | (6S)-4-(8-(6-chloro-5-(1- fluorocyclopropyl)-1H- indazol-4-yl)-2-((2- (dimethylamino)-4,4- difluorobicy- clo[3.1.0]hexan- 1-yl)methoxy)pyri- do[4′,3′:4,5]thi- eno[2,3-djpyrimidin-4-yl)- 6-methyl-1,4-oxazepan-6- ol | 714 |
| Ex- 272 | 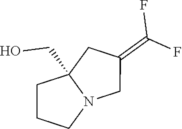 [SMILES: OC[C@@]12CCCN1CC(=C(F)F)C2] | 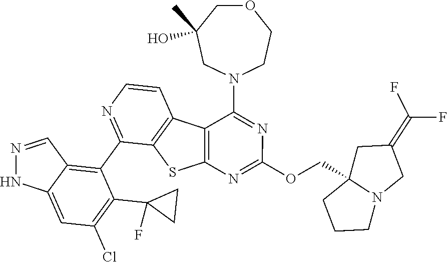 [SMILES: C[C@@]1(O)COCCN(c2nc(OC[C@@]34CCCN3CC(=C(F)F)C4)nc3sc4c(-c5c(C6(F)CC6)c(Cl)cc6[nH]ncc56)nccc4c23)C1] | (6S)-4-(8-(6-chloro-5-(1- fluorocyclopropyl)-1H- indazol-4-yl)-2-(((S)-2- (difluoromethylene)tetra- hydro- 1H-pyrrolizin-7a(5H)- yl)methoxy)pyri- do[4′,3′:4,5]thi- eno[2,3-d]pyrimidin-4-yl)- 6-methyl-1,4-oxazepan-6- ol | 712 |

### Example 273: (6S)-4-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[3′,4′:4,5]thieno[2,3-d]pyrimidin-4-yl)-6-methyl-1,4-oxazepan-6-ol (Ex-273)

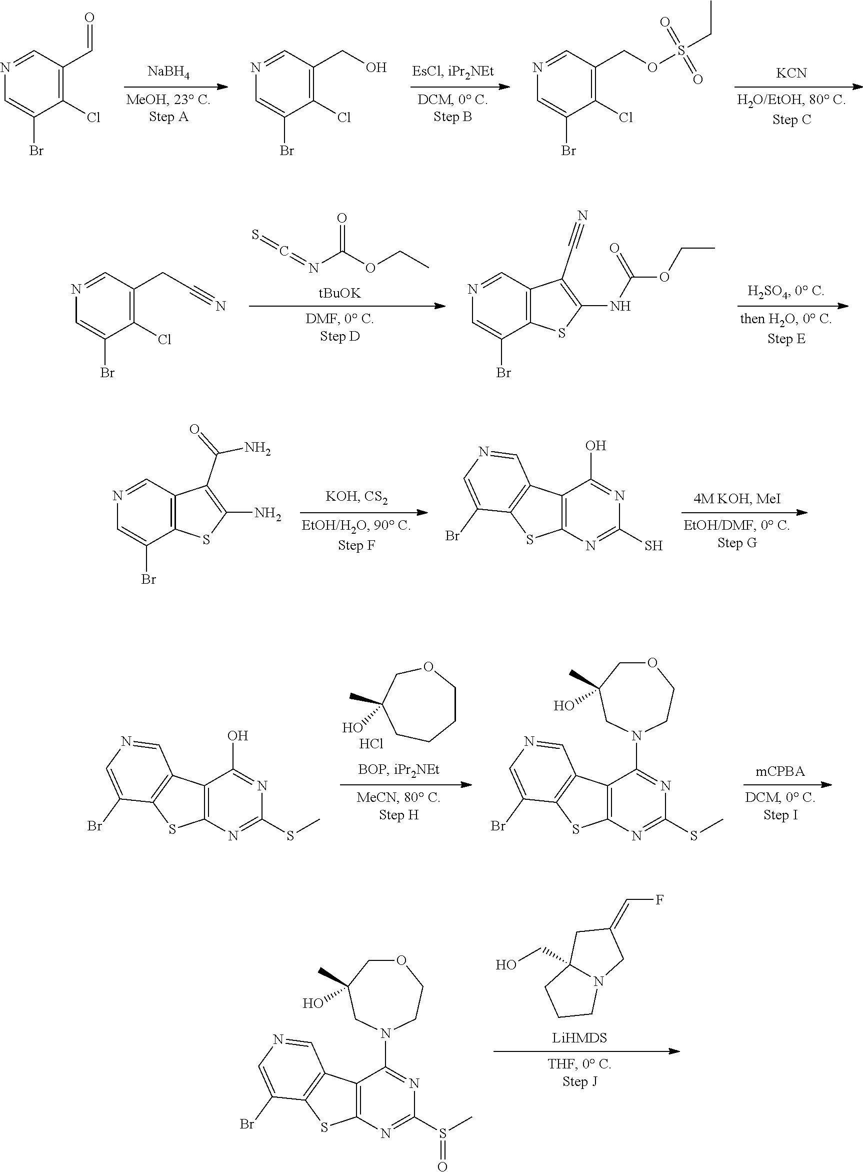

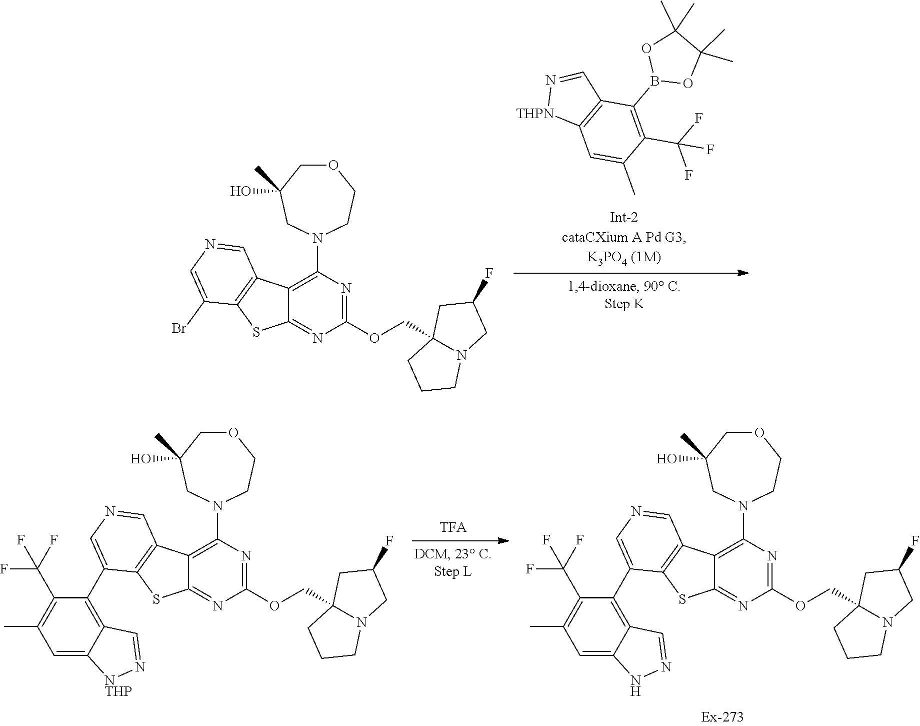

### Step A: (5-bromo-4-chloropyridin-3-yl) methanol

A 100 mL round bottom flask with a stir bar was charged with 5-bromo-4-chloronicotinaldehyde (2.0 g, 9.1 mmol) and MeOH (30 mL). The mixture was cooled to 0° C. and sodium borohydride (340 mg, 9.1 mmol) was added portionwise. The reaction mixture was warmed to r.t. After stirring the mixture at r.t. for 15 min, the reaction mixture was quenched by the addition of H₂O. The reaction mixture was extracted with EtOAc (3×20 mL). The combined organic layer was washed with brine (20 mL), dried over sodium sulfate, filtered, and concentrated under reduced pressure to give (5-bromo-4-chloropyridin-3-yl) methanol, which was used directly in the next step without purification. MS (ESI): m/z (M+H)⁺ 221, 223, 225. ¹H NMR (400 MHz, CDCl₃) δ 8.71 (s, 1H), 8.61 (s, 1H), 4.86 (d, J=6.4 Hz, 2H), 2.06 (t, J=6.4 Hz, 1H).

### Step B: (5-bromo-4-chloropyridin-3-yl)methyl ethanesulfonate

A 100 mL round bottom flask with a stir bar was charged with (5-bromo-4-chloropyridin-3-yl) methanol (2.00 g, 9.08 mmol) and DCM (30 mL). To a mixture was added N,N-diisopropylethylamine (3.10 mL, 18.2 mmol). The solution was cooled to 0° C. and ethanesulfonyl chloride (0.946 mL, 9.98 mmol) was added dropwise. The reaction mixture was warmed to r.t. After stirring the mixture at r.t. for 10 min, the reaction mixture was quenched by the addition of H₂O. The reaction mixture was extracted with CHCl₃ (3×20 mL). The combined organic layer was washed with brine (20 mL), dried over sodium sulfate, filtered, and concentrated under reduced pressure to give (5-bromo-4-chloropyridin-3-yl)methyl ethanesulfonate, which was used directly in the next step without further purification. MS (ESI): m/z (M+H)⁺ 313, 315, 317.

### Step C: 2-(5-bromo-4-chloropyridin-3-yl) acetonitrile

A 300 mL round bottom flask with a stir bar was charged with potassium cyanide (650 mg, 9.98 mmol) and H₂O (15 mL). The reaction mixture was warmed to 80° C. To the mixture was added the solution of crude (5-bromo-4-chloropyridin-3-yl)methyl ethanesulfonate from step B in EtOH (15 mL) at 80° C. After stirring the mixture at 80° C. for 15 min, the reaction mixture was cooled to r.t. and quenched by the addition of H₂O. The reaction mixture was extracted with EtOAc (3×20 mL). The combined organic layer was washed with brine (20 mL), dried over sodium sulfate, filtered, and concentrated under reduced pressure. The residue was purified by silica gel column (20 to 60% EtOAc in hexanes) to give 2-(5-bromo-4-chloropyridin-3-yl) acetonitrile. MS (ESI): m/z (M+H)⁺ 230, 232, 234. ¹H NMR (400 MHz, CDCl₃) δ 8.78 (s, 1H), 8.61 (s, 1H), 3.88 (s, 2H).

### Step D: ethyl N-(7-bromo-3-cyano-thieno[3,2-c]pyridin-2-yl) carbamate

A 50 mL round bottom flask with a stir bar was charged with 2-(5-bromo-4-chloropyridin-3-yl) acetonitrile (1.66 g, 7.17 mmol) and DMF (14 mL). The mixture was cooled to 0° C. and potassium tert-butoxide (845 mg, 7.53 mmol) was added portionwise. The reaction mixture was stirred at 0° C. for 30 min, and then ethoxycarbonyl isothiocyanate (0.890 mL, 7.53 mmol) was added at 0° C. The reaction mixture was stirred at 0° C. for 10 min. H₂O was added slowly with stirring. The resulting precipitate was collected by filtration, rinsed with H₂O and hexane, and dried under reduced pressure to give ethyl N-(7-bromo-3-cyano-thieno[3,2-c]pyridin-2-yl) carbamate. MS (ESI): m/z (M+H)⁺ 326, 328. ¹H NMR (400 MHz, DMSO-d₆) δ 12.4 (br, 1H), 8.74 (s, 1H), 8.50 (s, 1H), 4.20 (q, J=6.8 Hz, 2H), 1.21 (q. J=6.8 Hz, 3H).

### Step E: 2-amino-7-bromothieno[3,2-c]pyridine-3-carboxamide

A 300 mL round bottom flask with a stir bar was charged with sulfuric acid (2.50 mL). The reaction mixture was cooled to 0° C. To the mixture was added ethyl N-(7-bromo-3-cyano-thieno[3,2-c]pyridin-2-yl) carbamate (500 mg, 1.53 mmol) portionwise at 0° C. After stirring the mixture at 0° C. for 1 min, cold H₂O (2.50 mL) was added at 0° C. and then the reaction mixture was stirred at 0° C. for 10 min. LCMS indicated the desired product formed. The reaction mixture was neutralized with NaOH (17 mL, 100 mmol, 6 M in H₂O). After the addition of H₂O (50 mL) and THF (50 mL), the reaction mixture was extracted with THF (3×20 mL). The combined organic layer was washed with brine (20 mL), dried over sodium sulfate, filtered, and concentrated under reduced pressure to give 2-amino-7-bromothieno[3,2-c]pyridine-3-carboxamide. MS (ESI): m/z (M+H)⁺ 272, 274. ¹H NMR (400 MHz, DMSO-d₆) δ 8.74 (s, 1H), 8.18 (s, 1H), 7.94 (br, 2H), 7.12 (br, 2H).

### Step F: 8-bromo-2-mercaptopyrido[3′,4′:4,5]thieno[2,3-d]pyrimidin-4-ol

A 50 mL round bottom flask with a stir bar was charged with 2-amino-7-bromothieno[3,2-c]pyridine-3-carboxamide (145 mg, 0.533 mmol), EtOH (3.50 mL), H₂O (1.8 mL), and carbon disulfide (1.10 mL, 17.5 mmol). To this mixture was added 4M aq. KOH (0.160 mL, 0.639 mmol), and then the reaction mixture was heated to 90° C. for 30 min. After the reaction mixture was cooled to room temperature, KOH (0.320 mL, 1.28 mmol, 4.0 M in H₂O) was added, and then the reaction mixture was stirred at 90° C. for 1 h. After the reaction mixture was cooled to room temperature, additional KOH (0.160 mL, 0.639 mmol, 4.0 M in H₂O) was added, and then the reaction mixture was stirred at 90° C. for 7 h. After the reaction mixture was cooled to room temperature, HCl (1.30 mL, 2.60 mmol, 2 M in H₂O) was added and the reaction mixture was stirred at r.t. for 10 min. H₂O was added slowly with stirring. The resulting precipitate was collected by filtration, rinsed with H₂O and hexane, and dried under reduced pressure to give 8-bromo-2-mercaptopyrido[3′,4′:4,5]thieno[2,3-d]pyrimidin-4-ol. MS (ESI): m/z (M+H)⁺ 313.9, 315.9. ¹H NMR (400 MHz, DMSO-d₆) δ 13.8 (br, 1H), 12.9 (s, 1H), 9.23 (s, 1H), 8.58 (s, 1H).

### Step G: 8-bromo-2-(methylthio)pyrido[3′,4′:4,5]thieno[2,3-d]pyrimidin-4-ol

A 50 mL round bottom flask with a stir bar was charged with 8-bromo-2-mercaptopyrido[3′,4′:4,5]thieno[2,3-d]pyrimidin-4-ol (146 mg, 0.465 mmol), DMF (1.60 mL), and EtOH (0.470 mL). After the addition of KOH (0.120 mL, 0,465 mmol, 4.0 M in H₂O), the reaction mixture was cooled to 0° C. To this mixture was added MeI (34.8 μL, 0.558 mmol), and then the reaction mixture was stirred at 0° C. for 1 h. The reaction mixture was neutralized with HCl (240 μL, 2 M in H₂O), and then H₂O was added slowly with stirring. The resulting precipitate was collected by filtration, rinsed with H₂O and hexane, and dried under reduced pressure to give 8-bromo-2-(methylthio)pyrido[3′,4′:4,5]thieno[2,3-d]pyrimidin-4-ol. MS (ESI): m/z (M+H)⁺ 328, 330. ¹H NMR (400 MHz, DMSO-d₆) δ 13.4 (br, 1H), 9.30 (s, 1H), 8.61 (s, 1H), 2.52 (s, 3H).

### Step H: (S)-4-(8-bromo-2-(methylthio)pyrido[3′,4′:4,5]thieno[2,3-d]pyrimidin-4-yl)-6-methyl-1,4-oxazepan-6-ol

A test tube with a stir bar was charged with BOP (101.1 mg, 0.229 mmol), (6S)-6-methyl-1,4-oxazepan-6-ol hydrochloride (30.6 mg, 0.183 mmol), 8-bromo-2-(methylthio)pyrido[3′,4′:4,5]thieno[2,3-d]pyrimidin-4-ol (50.0 mg, 0.152 mmol), MeCN (1.5 mL) and N,N-diisopropylethylamine (78.1 μL, 0.457 mmol). After stirring the mixture at 80° C. for 15 min, the reaction mixture was cooled to r.t. and quenched by the addition of H₂O. The reaction mixture was extracted with EtOAc (3×20 mL). The combined organic layer was washed with brine (20 mL), dried over sodium sulfate, filtered, and concentrated under reduced pressure. The residue was purified by silica gel chromatography (30 to 100% EtOAc in hexanes) to give(S)-4-(8-bromo-2-(methylthio)pyrido[3′,4′:4,5]thieno[2,3-d]pyrimidin-4-yl)-6-methyl-1,4-oxazepan-6-ol. MS (ESI): m/z (M+H)⁺ 441, 443. ¹H NMR (400 MHz, DMSO-d₆) δ 8.91 (s, 1H), 8.63 (s, 1H), 4.66 (s, 1H), 3.90-3.69 (m, 8H), 2.48 (s, 3H), 0.80 (s, 3H).

### Step I: (6S)-4-(8-bromo-2-(methylsulfinyl)pyrido[3′,4′:4,5]thieno[2,3-d]pyrimidin-4-yl)-6-methyl-1,4-oxazepan-6-ol

A 100 mL round bottom flask with a stir bar was charged with (S)-4-(8-bromo-2-(methylthio)pyrido[3′,4′:4,5]thieno[2,3-d]pyrimidin-4-yl)-6-methyl-1,4-oxazepan-6-ol (63.8 mg, 0.145 mmol) and DCM (1.5 mL). The reaction mixture was cooled to 0° C. and mCPBA (33.3 mg, 0.145 mmol, 75% w/w) was added at 0° C. After stirring the mixture at 0° C. for 3 min, the reaction mixture was quenched by the addition of sat. aq. NaHCO₃. The reaction mixture was extracted with CHCl₃ (3×20 mL). The combined organic layer was washed with brine (20 mL), dried over sodium sulfate, filtered, and concentrated under reduced pressure to give (6S)-4-(8-bromo-2-(methylsulfinyl)pyrido[3′,4′:4,5]thieno[2,3-d]pyrimidin-4-yl)-6-methyl-1,4-oxazepan-6-ol, which was used directly in the next step without further purification. MS (ESI): m/z (M+H)⁺ 457, 459.

### Step J: (S)-4-(18-bromo-2-(2R,7aS)-2-fluorotetrahydro-1H-pyrrolizin-7a(5H)-yl)methoxy)pyrido[3′,4′:4,5]thieno[2,3-d]pyrimidin-4-yl)-6-methyl-1,4-oxazepan-6-ol

A 50 mL round bottom flask with a stir bar was charged with ((2R,7aS)-2-fluorotetrahydro-1H-pyrrolizin-7a(5H)-yl) methanol (34.5 mg, 0.217 mmol) and THF (1.5 mL). The reaction mixture was cooled at 0° C., and then LiHMDS (0.289 mL, 0.289 mmol, 1.00 M in THF) was added at 0° C. After stirring the mixture at 0° C. for 5 min, the solution of (6S)-4-(8-bromo-2-(methylsulfinyl)pyrido[3′,4′:4,5]thieno[2,3-d]pyrimidin-4-yl)-6-methyl-1,4-oxazepan-6-ol in THF (3.0 mL) was added to the reaction mixture at 0° C. and the reaction mixture was stirred at 0° C. for 1 min. The reaction mixture was quenched by the addition of H₂O and extracted with EtOAc (3×20 mL). The combined organic layer was washed with brine (20 mL), dried over sodium sulfate, filtered, and concentrated under reduced pressure. The residue was purified by silica gel chromatography (0 to 20% MeOH in CHCl₃) to give(S)-4-(8-bromo-2-(((2R,7aS)-2-fluorotetrahydro-1H-pyrrolizin-7a(5H)-yl)methoxy)pyrido[3′,4′:4,5]thieno[2,3-d]pyrimidin-4-yl)-6-methyl-1,4-oxazepan-6-ol. MS (ESI): m/z (M+H)⁺ 552, 554. ¹H NMR (400 MHz, CDCl₃) δ 8.85 (s, 1H), 8.61 (s, 1H), 8.32 (br, 1H), 5.36-5.21 (m, 1H), 4.28-4.18 (m, 2H), 4.09-4.04 (m, 2H), 3.96-3.85 (m, 1H), 3.77 (d, J=12.4 Hz, 1H), 3.72-3.64 (m, 1H), 3.60 (d, J=15.0 Hz, 1H), 3.38-3.13 (m, 5H), 3.00-2.95 (m, 1H), 2.23-2.10 (m, 2H), 1.98-1.80 (m, 4H), 1.35 (s, 3H).

### Step K: (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[3′,4′:4,5]thieno[2,3-d]pyrimidin-4-yl)-6-methyl-1,4-oxazepan-6-ol

A 50 mL round bottom flask with a stir bar was charged with (S)-4-(8-bromo-2-(((2R,7aS)-2-fluorotetrahydro-1H-pyrrolizin-7a(5H)-yl)methoxy)pyrido[3′,4′:4,5]thieno[2,3-d]pyrimidin-4-yl)-6-methyl-1,4-oxazepan-6-ol (27.2 mg, 0.0492 mmol), cataCXium A Pd G3 (10.8 mg, 0.0148 mmol), 6-methyl-1-(tetrahydro-2H-pyran-2-yl)-4-(4,4,5,5-tetramethyl-1,3,2-dioxaborolan-2-yl)-5-(trifluoromethyl)-1H-indazole (Int-2) (30.3 mg, 0.0738 mmol), K₃PO₄ (0.246 mL, 0.246 mmol, 1.00 M in H₂O) and 1,4-dioxane (0.985 mL). The mixture was degassed under reduced pressure and purged with nitrogen five times. The mixture was stirred at 90° C. for 4 h under nitrogen atmosphere. The reaction mixture was cooled to r.t. and diluted with water. The reaction mixture was extracted with EtOAc (3×20 mL). The combined organic layer was washed with brine (20 mL), dried over sodium sulfate, filtered, and concentrated under reduced pressure. (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[3′,4′:4,5]thieno[2,3-d]pyrimidin-4-yl)-6-methyl-1,4-oxazepan-6-ol was used directly in the next step without further purification. MS (ESI): m/z (M+H)⁺ 756.

### Step L: (6S)-4-(2-(((2R,7aS)-2-fluorotetrahydro-1H-pyrrolizin-7a(5H)-yl) methoxy)-8-(6-methyl-5-(trifluoromethyl)-1H-indazol-4-yl)pyrido[3′,4′:4,5]thieno[2,3-d]pyrimidin-4-yl)-6-methyl-1,4-oxazepan-6-ol (Ex-77)

A 20 mL round bottom flask with stir bar was charged with (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[3′,4′:4,5]thieno[2,3-d]pyrimidin-4-yl)-6-methyl-1,4-oxazepan-6-ol (37.2 mg, 0.0492 mmol) and DCM (0.5 mL). To the mixture was added TFA (0.183 mL, 2.46 mmol), and the mixture was stirred at r.t. for 30 min. The reaction mixture was evaporated and the residue was purified by preparative reverse phase HPLC (MeCN/water with 0.1% formic acid) to obtain (6S)-4-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[3′,4′:4,5]thieno[2,3-d]pyrimidin-4-yl)-6-methyl-1,4-oxazepan-6-ol (Ex-273). MS (ESI): m/z (M+H)⁺ 672. ¹H NMR (400 MHz, CDCl₃) δ 10.3 (br, 1H), 9.03 (s, 0.5H), 9.20 (s, 0.5H), 8.47 (s, 0.5H) 8.46 (s, 0.5H), 7.57 (s, 1H), 7.54 (s, 0.5H), 7.40 (s, 0.5H), 6.56 (br, 1H), 5.32-5.19 (m, 1H), 4.21-4.19 (m, 2H), 4.11-4.08 (m, 2H), 4.00-3.96 (m, 1H), 3.83-3.64 (m, 3H), 3.45˜3.32 (m, 2H), 3.28-3.19 (m, 2H), 3.13-3.08 (m, 1H), 3.00-2.95 (m, 1H), 2.74-2.73 (m, 3H), 2.23-2.03 (m, 2H), 1.96-1.82 (m, 4H), 1.39 (s, 3H).

### Example 274:2-amino-7-fluoro-4-(2-(((2R,7aS)-2-fluorotetrahydro-1H-pyrrolizin-7a(5H)-yl)methoxy)-4-((S)-6-hydroxy-6-methyl-1,4-oxazepan-4-yl)pyrido[3′,4′:4,5]thieno[2,3-d]pyrimidin-8-yl)benzo[b]thiophene-3-carbonitrile (Ex-274)

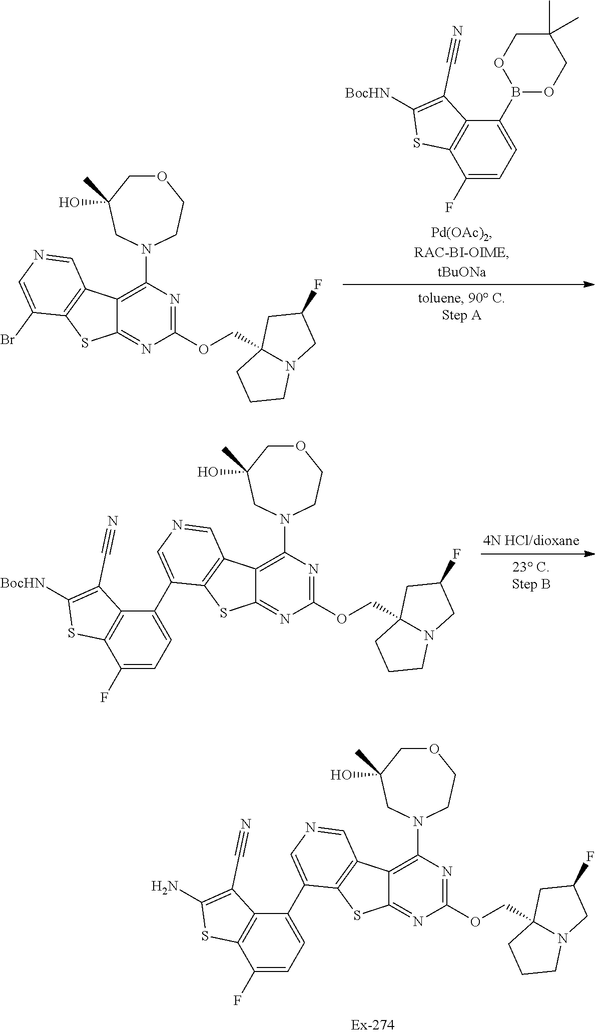 [SMILES: C[C@@]1(O)COCCN(c2nc(OC[C@@]34CCCN3C[C@H](F)C4)nc3sc4c(Br)cncc4c23)C1 ; CC1(C)COB(c2ccc(F)c3sc(NC(=O)OC(C)(C)C)c(C#N)c23)OC1 ; CC(C)(C)OC(=O)Nc1sc2c(F)ccc(-c3cncc4c3sc3nc(OC[C@@]56CCCN5C[C@H](F)C6)nc(N5CCOC[C@@](C)(O)C5)c34)c2c1C#N ; C[C@@]1(O)COCCN(c2nc(OC[C@@]34CCCN3C[C@H](F)C4)nc3sc4c(-c5ccc(F)c6sc(N)c(C#N)c56)cncc4c23)C1]

### Step A: Ter-butyl(3-cyano-7-fluoro-4-(2-(((2R,7aS)-2-fluorotetrahydro-1H-pyrrolizin-7a(5H)-yl)methoxy)-4-((S)-6-hydroxy-6-methyl-1,4-oxazepan-4-yl)pyrido[3′,4′:4,5]thieno[2,3-d]pyrimidin-8-yl)benzo[b]thiophen-2-yl) carbamate

A 20 mL round bottom flask with a stir bar was charged with (S)-4-(8-bromo-2-(((2R,7aS)-2-fluorotetrahydro-1H-pyrrolizin-7a(5H)-yl)methoxy)pyrido[3′,4′:4,5]thieno[2,3-d]pyrimidin-4-yl)-6-methyl-1,4-oxazepan-6-ol (30 mg, 0.056 mmol), Pd(OAc)₂ (10 mg, 0.045 mmol), rac-3-(tert-butyl)-4-(2,6-dimethoxyphenyl)-2,3-dihydrobenzo[d][1,3]oxaphosphole (30 mg, 0.090 mmol), sodium tert-butoxide (16 mg, 0.17 mmol), tert-butyl(3-cyano-4-(5,5-dimethyl-1,3,2-dioxaborinan-2-yl)-7-fluorobenzo[b]thiophen-2-yl) carbamate (57 mg, 0.14 mmol) and toluene (2.8 mL). The mixture was degassed under reduced pressure and purged with nitrogen five times. The mixture was stirred at 90° C. for 1.5 h under nitrogen atmosphere. The reaction mixture was cooled to r.t. and diluted with water. The reaction mixture was extracted with EtOAc (3×20 mL). The combined organic layer was washed with brine (20 mL), dried over sodium sulfate, filtered and concentrated under reduced pressure. tert-butyl(3-cyano-7-fluoro-4-(2-(((2R,7aS)-2-fluorotetrahydro-1H-pyrrolizin-7a(5H)-yl)methoxy)-4-((S)-6-hydroxy-6-methyl-1,4-oxazepan-4-yl)pyrido[3′,4′:4,5]thieno[2,3-d]pyrimidin-8-yl)benzo[b]thiophen-2-yl) carbamate was used directly in the next step without further purification. MS (ESI): m/z (M+H)⁺ 764.

### Step B: 2-amino-7-fluoro-4-(2-(((2R,7aS)-2-fluorotetrahydro-1H-pyrrolizin-7a(5H)-yl)methoxy)-4-((S)-6-hydroxy-6-methyl-1,4-oxazepan-4-yl)pyrido[3′,4′:4,5]thieno[2,3-d]pyrimidin-8-yl)benzo[b]thiophene-3-carbonitrile (Ex-274)

To tert-butyl(3-cyano-7-fluoro-4-(2-(((2R,7aS)-2-fluorotetrahydro-1H-pyrrolizin-7a(5H)-yl)methoxy)-4-((S)-6-hydroxy-6-methyl-1,4-oxazepan-4-yl)pyrido[3′,4′:4,5]thieno[2,3-d]pyrimidin-8-yl)benzo[b]thiophen-2-yl) carbamate (43 mg, 0.056 mmol) was added HCl (1.4 mL, 4.0 M in dioxane), and the mixture was stirred at r.t. for 30 min. The reaction mixture was evaporated and the residue was purified by reverse phase HPLC (MeCN/water with 0.1% formic acid), and then purified by silica gel NH₂ column (0% to 40% MeOH in EtOAc) to obtain 2-amino-7-fluoro-4-(2-(((2R,7aS)-2-fluorotetrahydro-1H-pyrrolizin-7a(5H)-yl)methoxy)-4-((S)-6-hydroxy-6-methyl-1,4-oxazepan-4-yl)pyrido[3′,4′:4,5]thieno[2,3-d]pyrimidin-8-yl)benzo[b]thiophene-3-carbonitrile (Ex-274). MS (ESI): m/z (M+H)⁺ 664. ¹H NMR (400 MHz, CDCl₃) δ 9.0 (m, 1H), 8.50 (m, 1H), 7.40-7.33 (m, 1H), 7.09-7.04 (m, 1H), 6.45-6.19 (m, 1H), 5.32-5.19 (m, 3H), 4.22-4.09 (m, 4H), 3.97-3.87 (m, 1H), 3.79-3.60 (m, 3H), 3.47-3.36 (m, 1H), 3.35-3.29 (m, 1H), 3.28-3.20 (m, 2H), 3.16-3.09 (m, 1H), 3.00-2.94 (m, 1H), 2.23-2.02 (m, 4H), 1.95-1.84 (m, 2H), 1.36-1.35 (m, 3H).

The example in the table below was synthesized using a similar procedure as described in the synthesis of Ex-274 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | | [M + |
|---|---|---|---|---|
| | | | | H]⁺ |
| Ex. | Starting Material | Structure | Compound Name | Found |
| Ex- 275 | 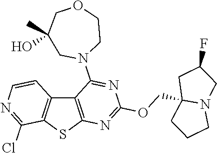 [SMILES: C[C@@]1(O)COCCN(c2nc(OC[C@@]34CCCN3C[C@H](F)C4)nc3sc4c(Cl)nccc4c23)C1] Int-143 | 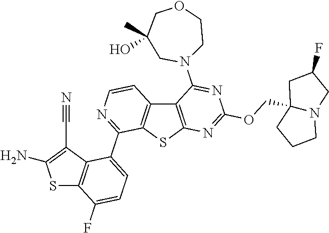 [SMILES: C[C@@]1(O)COCCN(c2nc(OC[C@@]34CCCN3C[C@H](F)C4)nc3sc4c(-c5ccc(F)c6sc(N)c(C#N)c56)nccc4c23)C1] | 2-amino-7-fluoro-4-(2- (((2R,7aS)-2-fluoro- tetrahydro- 1H-pyrrolizin-7a(5H)- yl)methoxy)-4-((S)-6- hydroxy-6-methyl- 1,4-oxazepan-4-yl) pyrido[4′,3′:4,5]thi- eno[2,3-d]pyrimidin-8- yl)benzo[b]thiophene-3- carbonitrile | 664 |

### Example 276: (6S)-4-(7-(((2R,7aS)-2-fluorotetrahydro-1H-pyrrolizin-7a(5H)-yl)methoxy)-4-(6-methyl-5-(trifluoromethyl)-1H-indazol-4-yl) thieno[2,3-d:4,5-d′]dipyrimidin-9-yl)-6-methyl-1,4-oxazepan-6-ol (Ex-276)

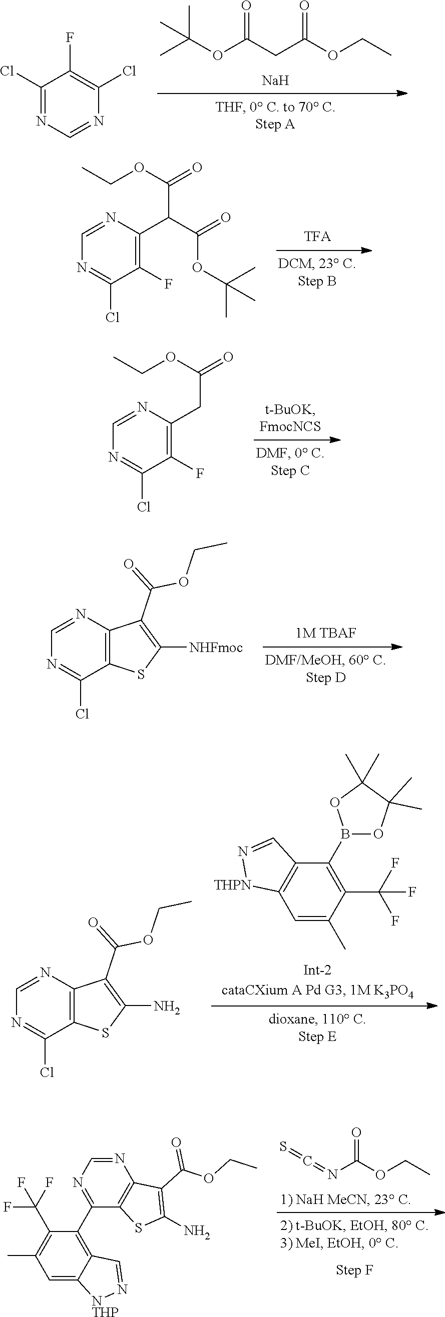

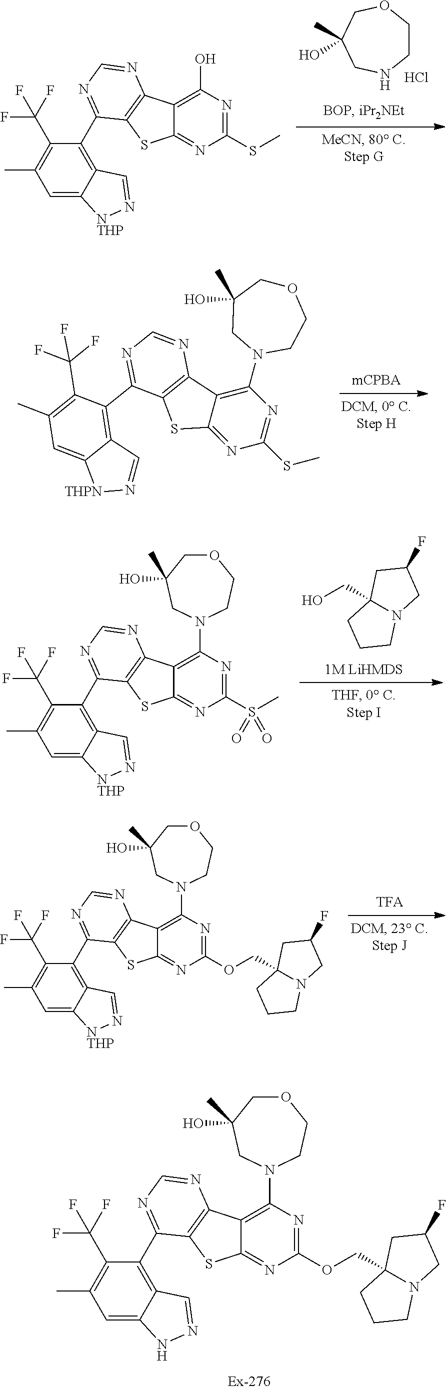

### Step A: 1-(ter-butyl)-3-ethyl 2-(6-chloro-5-fluoropyrimidin-4-yl) malonate

A 300 mL round bottom flask with a stir bar was charged with NaH (1.08 g, 27.0 mmol, 60 wt % in mineral oil) and THF (60 mL). The reaction mixture was cooled to 0° C. To this mixture was added tert-butyl ethyl malonate (3.74 mL, 19.79 mmol) dropwise via a syringe at 0° C. After stirring the mixture at 0° C. for 40 min, 4,6-dichloro-5-fluoropyrimidine (3.00 g, 18.0 mmol) was added to the reaction mixture at 0° C. The reaction mixture was stirred at 0° C. for 5 min, and then stirred at 70° C. for 20 min. The reaction mixture was cooled to r.t. and quenched by the addition of H₂O and 6 M aq. HCl (5 mL). The reaction mixture was extracted with EtOAc (3×20 mL). The combined organic layer was washed with brine (20 mL), dried over sodium sulfate, filtered, and concentrated under reduced pressure to give 1-(tert-butyl)-3-ethyl 2-(6-chloro-5-fluoropyrimidin-4-yl) malonate as a crude material, which was used directly in the next step without further purification. MS (ESI): m/z (M+H)⁺ 319.

### Step B: Ethyl 2-(6-chloro-5-fluoropyrimidin-4-yl)acetate

A 300 mL round bottom flask with a stir bar was charged with 1-(tert-butyl)-3-ethyl 2-(6-chloro-5-fluoropyrimidin-4-yl) malonate and DCM (9 mL). To this reaction mixture was added TFA (9 mL) and the reaction mixture was stirred at r.t. for 3 h. The reaction mixture was concentrated and the residue was diluted with water and EtOAc. The mixture was extracted with EtOAc (3×20 mL). The combined organic layer was washed with brine (20 mL), dried over sodium sulfate, filtered, and concentrated under reduced pressure. The residue was purified by silica gel chromatography (10 to 50% EtOAc in hexane) to give ethyl 2-(6-chloro-5-fluoropyrimidin-4-yl)acetate. MS (ESI): m/z (M+H)⁺ 219, 221. ¹H NMR (400 MHz, CDCl₃) δ 8.73 (s, 1H), 4.22 (q, J=7.2 Hz, 2H), 3.93 (d, J=2.0 Hz, 2H), 1.275 (t, J=7.2 Hz, 3H).

### Step C: Ethyl 6-((((9H-fluoren-9-yl)methoxy) carbonyl)amino)-4-chlorothieno[3,2-d]pyrimidine-7-carboxylate

A 50 mL round bottom flask with a stir bar was charged with ethyl 2-(6-chloro-5-fluoropyrimidin-4-yl)acetate (500 mg, 2.29 mmol) and DMF (4.6 mL). The mixture was cooled to 0° C. and potassium tert-butoxide (270 mg, 2.40 mmol) was added portionwise. The reaction mixture was stirred at 0° C. for 10 min, and then 9-fluorenylmethoxycarbonyl isothiocyanate (676 mg, 2.40 mmol) was added at 0° C. The reaction mixture was stirred at 0° C. for 5 min. H₂O was added slowly with stirring. The resulting precipitate was collected by filtration, rinsed with H₂O and hexane, and dried under reduced pressure to give ethyl 6-((((9H-fluoren-9-yl)methoxy) carbonyl)amino)-4-chlorothieno[3,2-d]pyrimidine-7-carboxylate. MS (ESI): m/z (M+H)⁺ 480, 482. ¹H NMR (400 MHz, DMSO-d₆) δ 11.2 (s, 1H), 8.82 (s, 1H), 7.82 (d. J=7.2 Hz, 2H), 7.64 (d, J=7.2 Hz, 2H), 7.38 (m, 2H), 7.26 (m, 2H), 4.59 (br, 2H), 4.27 (m, 3H), 1.22 (t, J=7.1 Hz, 3H).

### Step D: Ethyl 6-amino-4-chlorothieno[3,2-d]pyrimidine-7-carboxylate

A 50 mL round bottom flask with a stir bar was charged with ethyl 6-((((9H-fluoren-9-yl)methoxy) carbonyl)amino)-4-chlorothieno[3,2-d]pyrimidine-7-carboxylate (600 mg, 1.25 mmol), DMF (3.2 mL) and MeOH (1.6 mL). To the reaction mixture was added IM TBAF in THF (3.0 mL, 3.00 mmol) and the reaction mixture was stirred at 60° C. for 4 h. The reaction mixture was cooled to r.t. and quenched by the addition of H₂O. The mixture was extracted with MeOH/CHCl₃ (1:9) (3×20 mL). The combined organic layer was washed with a phosphate buffer solution (pH=6.8) and brine, dried over sodium sulfate, filtered, and concentrated under reduced pressure. The residue was purified by silica gel column (30 to 100% EtOAc in hexane) to give ethyl 6-amino-4-chlorothieno[3,2-d]pyrimidine-7-carboxylate. MS (ESI): m/z (M+H)⁺ 258, 260. ¹H NMR (400 MHz, DMSO-d₆) δ 8.82 (br, 2H), 8.26 (s, 1H), 4.21 (q, J=7.2 Hz, 2H), 1.20 (t, J=7.2 Hz, 3H).

### Step E: Ethyl 6-amino-4-(6-methyl-1-(tetrahydro-2H-pyran-2-yl)-5-(trifluoromethyl)-1H-indazol-4-yl) thieno[3,2-d]pyrimidine-7-carboxylate

A 50 mL round bottom flask with a stir bar was charged with ethyl 6-amino-4-chlorothieno[3,2-d]pyrimidine-7-carboxylate (46.6 mg, 0.181 mmol), cataCXium A Pd G3 (39.5 mg, 0.0542 mmol), 6-methyl-1-(tetrahydro-2H-pyran-2-yl)-4-(4,4,5,5-tetramethyl-1,3,2-dioxaborolan-2-yl)-5-(trifluoromethyl)-1H-indazole (Int-2) (89.0 mg, 0.217 mmol), K₃PO₄ (0.904 mL, 0.904 mmol, 1 M in H₂O) and 1,4-dioxane (3.6 mL). The mixture was degassed under reduced pressure and purged with nitrogen five times. The mixture was stirred at 110° C. for 2.5 h under nitrogen atmosphere. The reaction mixture was cooled to r.t. and additional cataCXium A Pd G3 (39.5 mg, 0.0542 mmol) and 6-methyl-1-(tetrahydro-2H-pyran-2-yl)-4-(4,4,5,5-tetramethyl-1,3,2-dioxaborolan-2-yl)-5-(trifluoromethyl)-1H-indazole (Int-2) (89.0 mg, 0.217 mmol) were added. The mixture was degassed under reduced pressure and purged with nitrogen five times. The mixture was stirred at 110° C. for 6 h under nitrogen atmosphere. The reaction mixture was cooled to r.t. and diluted with water and 2M HCl (1.8 mL). The reaction mixture was extracted with EtOAc (3×20 mL). The combined organic layer was washed with brine (20 mL), dried over sodium sulfate, filtered, and concentrated under reduced pressure. The residue was purified by silica gel column (30 to 100% EtOAc in hexane then 0% to 20% MeOH in EtOAc) to give ethyl 6-amino-4-(6-methyl-1-(tetrahydro-2H-pyran-2-yl)-5-(trifluoromethyl)-1H-indazol-4-yl) thieno[3,2-d]pyrimidine-7-carboxylate. MS (ESI): m/z (M+H)⁺ 506.

### Step F: 4-(6-methyl-1-(tetrahydro-2H-pyran-2-yl)-5-(trifluoromethyl)-1H-indazol-4-yl)-7-(methylthio) thieno[2,3-d:4,5-d′]dipyrimidin-9-ol

A 50 mL round bottom flask with a stir bar was charged with ethyl 6-amino-4-(6-methyl-1-(tetrahydro-2H-pyran-2-yl)-5-(trifluoromethyl)-1H-indazol-4-yl) thieno[3,2-d]pyrimidine-7-carboxylate (54.1 mg, 0.107 mmol) and MeCN (2.1 mL). The reaction mixture was cooled to 0° C. To the mixture was added NaH (5.1 mg, 0.13 mmol, 60 wt % in mineral oil) at 0° C. After stirring the mixture at 0° C. for 5 min, ethoxycarbonyl isothiocyanate (18.9 μL, 0.161 mmol) was added to the reaction mixture at 0° C. After the reaction mixture was stirred at 0° C. for 50 min, MeOH was added and the reaction mixture was evaporated. To the residue was added potassium tert-butoxide (36.0 mg, 0.321 mmol) and EtOH (2.1 mL), and then the reaction mixture was stirred at 80° C. for 20 min. After the reaction mixture was cooled to 0° C., MeI (7.3 μL, 0.12 mmol) was added. The reaction mixture was stirred at 0° C. for 1 h. The reaction mixture was quenched by the addition of H₂O and 2M aq. HCl (0.30 mL). The reaction mixture was extracted with EtOAc (3×20 mL). The combined organic layer was washed with brine (20 mL), dried over sodium sulfate, filtered, and concentrated under reduced pressure. The residue was purified by silica gel column (30 to 100% EtOAc in Hexane then 0% to 20% MeOH in CHCl₃) to give 4-(6-methyl-1-(tetrahydro-2H-pyran-2-yl)-5-(trifluoromethyl)-1H-indazol-4-yl)-7-(methylthio) thieno[2,3-d: 4,5-d′]dipyrimidin-9-ol. This product was used directly in the next step without further purification. MS (ESI): m/z (M+H)⁺ 533.

### Step G: (6S)-6-methyl-4-(4-(6-methyl-1-(tetrahydro-2H-pyran-2-yl)-5-(trifluoromethyl)-1H-indazol-4-yl)-7-(methylthio) thieno[2,3-d: 4,5-d′]dipyrimidin-9-yl)-1,4-oxazepan-6-ol

A 50 mL round bottom flask with a stir bar was charged with BOP (20.3 mg, 0.0459 mmol), (6S)-6-methyl-1,4-oxazepan-6-ol hydrochloride (7.7 mg, 0.0459 mmol), 4-(6-methyl-1-(tetrahydro-2H-pyran-2-yl)-5-(trifluoromethyl)-1H-indazol-4-yl)-7-(methylthio) thieno[2,3-d: 4,5-d′]dipyrimidin-9-ol (16.3 mg, 0.031 mmol), MeCN (0.30 mL) and DIPEA (15.7 μL, 0.0918 mmol). After stirring the mixture at 80° C. for 15 min, the reaction mixture was cooled to r.t. To the mixture was added additional BOP (20.3 mg, 0.0459 mmol) and (6S)-6-methyl-1,4-oxazepan-6-ol hydrochloride (7.7 mg, 0.0459 mmol), and the reaction mixture was stirred at 80° C. for 4 h. The reaction mixture was cooled to r.t. and quenched by the addition of H₂O. The reaction mixture was extracted with EtOAc (3×20 mL). The combined organic layer was washed with brine (20 mL), dried over sodium sulfate, filtered, and concentrated under reduced pressure. The residue was purified by silica gel column (30 to 100% EtOAc in hexanes) to give (6S)-6-methyl-4-(4-(6-methyl-1-(tetrahydro-2H-pyran-2-yl)-5-(trifluoromethyl)-1H-indazol-4-yl)-7-(methylthio) thieno[2,3-d: 4,5-d′]dipyrimidin-9-yl)-1,4-oxazepan-6-ol. MS (ESI): m/z (M+H)⁺ 646.

### Step H: (6S)-6-methyl-4-(4-(6-methyl-1-(tetrahydro-2H-pyran-2-yl)-5-(trifluoromethyl)-1H-indazol-4-yl)-7-(methylsulfonyl) thieno[2,3-d: 4,5-d′]dipyrimidin-9-yl)-1,4-oxazepan-6-ol

A 50 mL round bottom flask with a stir bar was charged with (6S)-6-methyl-4-(4-(6-methyl-1-(tetrahydro-2H-pyran-2-yl)-5-(trifluoromethyl)-1H-indazol-4-yl)-7-(methylthio) thieno[2,3-d: 4,5-d′]dipyrimidin-9-yl)-1,4-oxazepan-6-ol (19.0 mg, 0.029 mmol) and DCM (0.98 mL). The reaction mixture was cooled to 0° C. and mCPBA (6.8 mg, 0.0294 mmol) was added at 0° C. After stirring the mixture at 0° C. for 10 min, the reaction mixture was quenched by the addition of sat aq. NaHCO₃. The reaction mixture was extracted with CHCl₃ (3×20 mL). The combined organic layer was washed with brine (20 mL), dried over sodium sulfate, filtered and concentrated under reduced pressure to give (6S)-6-methyl-4-(4-(6-methyl-1-(tetrahydro-2H-pyran-2-yl)-5-(trifluoromethyl)-1H-indazol-4-yl)-7-(methylsulfonyl) thieno[2,3-d: 4,5-d′]dipyrimidin-9-yl)-1,4-oxazepan-6-ol, which was used directly in the next step without further purification. MS (ESI): m/z (M+H)⁺ 678.

### Step I: (6S)-4-(7-(((2R,7aS)-2-fluorotetrahydro-1H-pyrrolizin-7a(5H)-yl)methoxy)-4-(6-methyl-1-(tetrahydro-2H-pyran-2-yl)-5-(trifluoromethyl)-1H-indazol-4-yl) thieno[2,3-d:4,5-d′]dipyrimidin-9-yl)-6-methyl-1,4-oxazepan-6-ol

A 50 mL round bottom flask with a stir bar was charged with ((2R,7aS)-2-fluorotetrahydro-1H-pyrrolizin-7a (5H)-yl) methanol (7.0 mg, 0.0441 mmol) and THF (0.3 mL). The reaction mixture was cooled at 0° C., and then IM LiHMDS (60 μL, 0.059 mmol) was added at 0° C. After stirring the mixture at 0° C. for 5 min, a solution of (6S)-6-methyl-4-(4-(6-methyl-1-(tetrahydro-2H-pyran-2-yl)-5-(trifluoromethyl)-1H-indazol-4-yl)-7-(methylsulfonyl) thieno[2,3-d: 4,5-d′]dipyrimidin-9-yl)-1,4-oxazepan-6-ol in THF (1.2 mL) was added to the reaction mixture at 0° C. and the reaction mixture was stirred at 0° C. for 1 min. The reaction mixture was quenched by the addition of H₂O and extracted with EtOAc (3×20 mL). The combined organic layer was washed with brine (20 mL), dried over sodium sulfate, filtered and concentrated under reduced pressure. The residue was purified by silica gel column (0 to 20% MeOH in CHCl₃) to give (6S)-4-(7-(((2R,7aS)-2-fluorotetrahydro-1H-pyrrolizin-7a(5H)-yl)methoxy)-4-(6-methyl-1-(tetrahydro-2H-pyran-2-yl)-5-(trifluoromethyl)-1H-indazol-4-yl) thieno[2,3-d: 4,5-d′]dipyrimidin-9-yl)-6-methyl-1,4-oxazepan-6-ol, which was used directly in the next step without purification. MS (ESI): m/z (M+H) 757.

### Step J: (6S)-4-(7-(((2R,7aS)-2-fluorotetrahydro-1H-pyrrolizin-7a(5H)-yl)methoxy)-4-(6-methyl-5-(trifluoromethyl)-1H-indazol-4-yl) thieno[2,3-d: 4,5-d′]dipyrimidin-9-yl)-6-methyl-1,4-oxazepan-6-ol (Ex-276)

A 20 mL round bottom flask with a stir bar was charged with (6S)-4-(7-(((2R,7aS)-2-fluorotetrahydro-1H-pyrrolizin-7a(5H)-yl)methoxy)-4-(6-methyl-1-(tetrahydro-2H-pyran-2-yl)-5-(trifluoromethyl)-1H-indazol-4-yl) thieno[2,3-d: 4,5-d′]dipyrimidin-9-yl)-6-methyl-1,4-oxazepan-6-ol and DCM (0.2 mL). To the mixture was added TFA (0.2 mL, 2.50 mmol), and the mixture was stirred at r.t. for 30 min. The reaction mixture was evaporated and the residue was purified by NH₂ silica gel column (0 to 40% MeOH in EtOAc) to give (6S)-4-(7-(((2R,7aS)-2-fluorotetrahydro-1H-pyrrolizin-7a(5H)-yl)methoxy)-4-(6-methyl-5-(trifluoromethyl)-1H-indazol-4-yl) thieno[2,3-d: 4,5-d′]dipyrimidin-9-yl)-6-methyl-1,4-oxazepan-6-ol (Ex-276). MS (ESI): m/z (M+H)⁺ 673. ¹H NMR (400 MHz, CDCl₃) δ 10.5 (br, 1H), 9.30 (s, 1H), 7.59 (s, 1H), 7.535 (m, 1H), 5.93 (br, 1H), 5.34-5.21 (m, 1H), 4.62-4.57 (m, 1H), 4.23-4.15 (m, 2H), 4.03-3.99 (m, 2H), 3.78-3.63 (m, 3H), 3.50-3.46 (m, 2H), 3.32-3.12 (m, 3H), 3.02-2.95 (m, 1H), 2.67-2.64 (m, 3H), 2.28-1.92 (m, 6H), 1.39-1.37 (m, 3H).

### Example 277: (6S)-4-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrimido[5′,4′:4,5]thieno[2,3-d]pyridazin-4-yl)-6-methyl-1,4-oxazepan-6-ol (Ex-277)

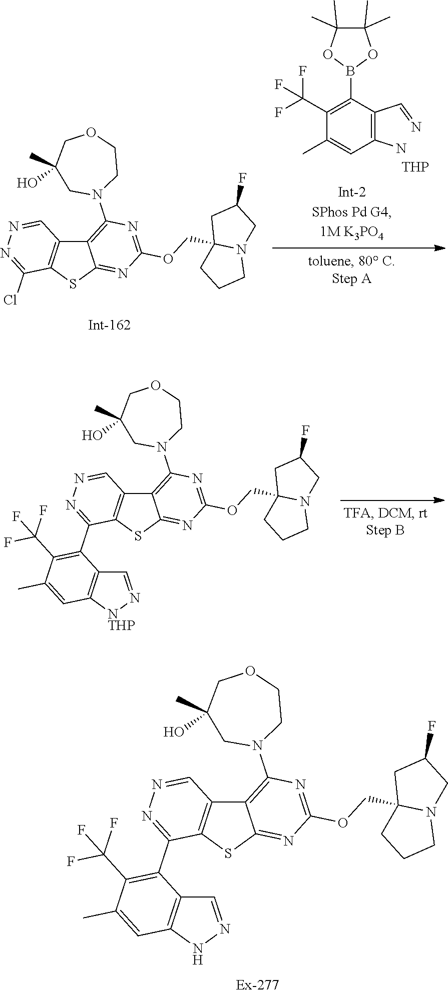

### Step A: (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrimido[5′,4′:4,5]thieno[2,3-d]pyridazin-4-yl)-6-methyl-1,4-oxazepan-6-ol

A mixture of(S)-4-(8-chloro-2-(((2R,7aS)-2-fluorotetrahydro-1H-pyrrolizin-7a(5H)-yl)methoxy)pyrimido[5′,4′:4,5]thieno[2,3-d]pyridazin-4-yl)-6-methyl-1,4-oxazepan-6-ol (Int-162) (20 mg, 0.039 mmol), 6-methyl-1-(tetrahydro-2H-pyran-2-yl)-4-(4,4,5,5-tetramethyl-1,3,2-dioxaborolan-2-yl)-5-(trifluoromethyl)-1H-indazole (Int-2) (19.3 mg, 0.047 mmol), SPhos Pd G4 (15.6 mg, 0.020 mmol), and K₃PO₄ (0.2 mL, 0.20 mmol, 1 M in H₂O) in toluene (0.80 ml) was degassed and nitrogen charged (3×). Then the mixture was stirred at 80° C. under nitrogen atmosphere for 5 h. After cooling, the mixture was partitioned between EtOAc and water. The organic phase was washed with brine, dried over sodium sulfate, filtered, and concentrated. The crude residue was purified via silica gel chromatography (0 to 10% MeOH in chloroform)) to give (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrimido[5′,4′:4,5]thieno[2,3-d]pyridazin-4-yl)-6-methyl-1,4-oxazepan-6-ol. This material was used directly in the next step without further purification. MS (ESI): m/z (M+H)⁺ 757.

### Step B: (6S)-4-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrimido[5′,4′:4,5]thieno[2,3-d]pyridazin-4-yl)-6-methyl-1,4-oxazepan-6-ol (Ex-277)

To a stirred solution of (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrimido[5′,4′:4,5]thieno[2,3-d]pyridazin-4-yl)-6-methyl-1,4-oxazepan-6-ol (10.9 mg, 0.014 mmol) in DCM (0.14 ml) was added TFA (72 μl). After 15 min, the reaction was evaporated and the residue was purified by preparative reverse-phase HPLC (MeCN/water with 0.1% formic acid) to obtain (6S)-4-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrimido[5′,4′:4,5]thieno[2,3-d]pyridazin-4-yl)-6-methyl-1,4-oxazepan-6-ol (Ex-277). MS (ESI): m/z (M+H)⁺ 673. ¹H NMR (400 MHz, CDCl₃) δ 9.53 (s, 1H), 7.63 (s, 1H), 7.50-7.43 (m, 1H), 6.21-5.90 (m, 1H), 5.35-5.22 (m, 1H), 4.32-4.12 (m, 5H), 4.02-3.99 (m, 1H), 3.92-3.83 (m, 1H), 3.83-3.80 (m, 1H), 3.72-3.64 (m, 2H), 3.59-3.52 (m, 1H), 3.38-3.34 (m, 1H), 3.24-3.19 (m, 1H), 3.02-2.98 (m, 1H), 2.75 (s, 3H), 2.30-2.05 (m, 4H), 1.98-1.90 (m, 2H), 1.37 (s, 3H).

The examples in the table below were synthesized using a similar procedure as described in the synthesis of Ex-277 by making the appropriate substitutions for starting material, intermediates, and or reagents. Such starting materials, intermediates, and/or reagents are available commercially, synthesized as described in the literature, synthesized using methods available to those skilled in the art, or synthesized as described herein.

| | | | | [M + H]⁺ |
|---|---|---|---|---|
| Ex. | Starting Material | Structure | Compound Name | Found |
| Ex 278 | 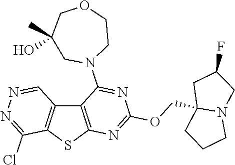 [SMILES: C[C@@]1(O)COCCN(c2nc(OC[C@@]34CCCN3C[C@H](F)C4)nc3sc4c(Cl)nncc4c23)C1] Int-162 | 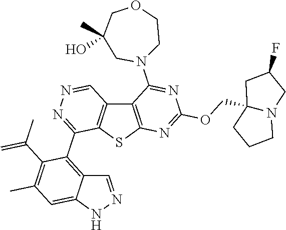 [SMILES: C=C(C)c1c(C)cc2[nH]ncc2c1-c1nncc2c1sc1nc(OC[C@@]34CCCN3C[C@H](F)C4)nc(N3CCOC[C@@](C)(O)C3)c12] | (6S)-4-(2-(((2R,7aS)-2- fluorotetrahydro-1H- pyrrolizin-7a(5H)- yl)methoxy)-8-(6-methyl- 5-(prop-1-en-2-y])-1H- indazol-4-yl)pyrimi- do[5′,4′:4,5]thieno [2,3-d]pyridazin-4-yl)-6- methyl-1,4-oxazepan-6-ol | 645 |
| Ex- 279 | 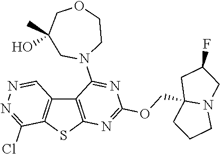 [SMILES: C[C@@]1(O)COCCN(c2nc(OC[C@@]34CCCN3C[C@H](F)C4)nc3sc4c(Cl)nncc4c23)C1] Int-162 | 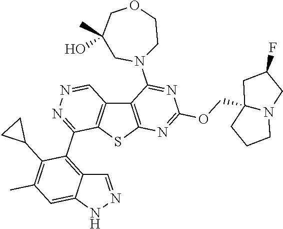 [SMILES: Cc1cc2[nH]ncc2c(-c2nncc3c2sc2nc(OC[C@@]45CCCN4C[C@H](F)C5)nc(N4CCOC[C@@](C)(O)C4)c23)c1C1CC1] | (6S)-4-(8-(5-cyclopropyl- 6-methyl-1H-indazol-4- yl)-2-(((2R,7aS)-2- fluorotetrahydro-1H- pyrrolizin-7a(5H)- yl)methoxy)pyrimi- do[5′,4′:4,5]thieno[2,3- d]pyridazin-4-yl)-6- methyl-1,4-oxazepan-6-ol | 645 |
| Ex- 280 | 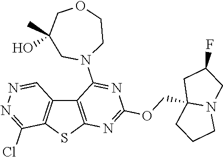 [SMILES: C[C@@]1(O)COCCN(c2nc(OC[C@@]34CCCN3C[C@H](F)C4)nc3sc4c(Cl)nncc4c23)C1] Int-162 | 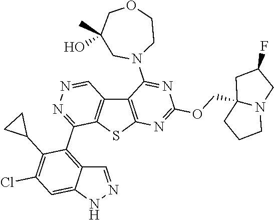 [SMILES: C[C@@]1(O)COCCN(c2nc(OC[C@@]34CCCN3C[C@H](F)C4)nc3sc4c(-c5c(C6CC6)c(Cl)cc6[nH]ncc56)nncc4c23)C1] | (6S)-4-(8-(6-chloro-5- cyclopropyl-1H-indazol- 4-yl)-2-(((2R,7aS)-2- fluorotetrahydro-1H- pyrrolizin-7a(5H)- yl)methoxy)pyrimi- do[5′,4′:4,5]thieno[2,3- d]pyridazin-4-yl)-6- methyl-1,4-oxazepan-6-ol | 665 |
| Ex- 281 | 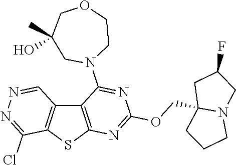 [SMILES: C[C@@]1(O)COCCN(c2nc(OC[C@@]34CCCN3C[C@H](F)C4)nc3sc4c(Cl)nncc4c23)C1] Int-162 | 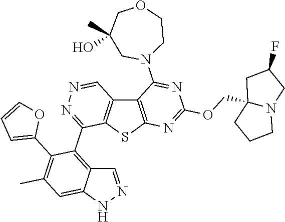 [SMILES: Cc1cc2[nH]ncc2c(-c2nncc3c2sc2nc(OC[C@@]45CCCN4C[C@H](F)C5)nc(N4CCOC[C@@](C)(O)C4)c23)c1-c1ccco1] | (6S)-4-(2-(((2R,7aS)-2- fluorotetrahydro-1H- pyrrolizin-7a(5H)- yl)methoxy)-8-(5-(furan- 2-yl)-6-methyl-1H- indazol-4- yl)pyrimido[5′,4′:4,5]thie- no[2,3-d]pyridazin-4-yl)-6- methyl-1,4-oxazepan-6-ol | 671 |
| Ex- 282 | 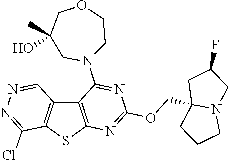 [SMILES: C[C@@]1(O)COCCN(c2nc(OC[C@@]34CCCN3C[C@H](F)C4)nc3sc4c(Cl)nncc4c23)C1] Int-162 | 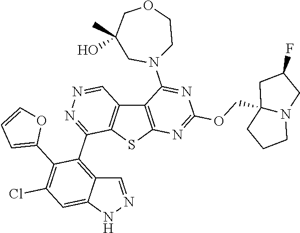 [SMILES: C[C@@]1(O)COCCN(c2nc(OC[C@@]34CCCN3C[C@H](F)C4)nc3sc4c(-c5c(-c6ccco6)c(Cl)cc6[nH]ncc56)nncc4c23)C1] | (6S)-4-(8-(6-chloro-5- (furan-2-yl)-1H-indazol- 4-yl)-2-(((2R,7aS)-2- fluorotetrahydro-1H- pyrrolizin-7a(5H)- yl)methoxy)pyrimi- do[5′,4′:4,5]thieno[2,3- dipyridazin-4-yl)-6- methyl-1,4-oxazepan-6-ol | 691 |
| Ex- 283 | 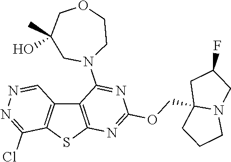 [SMILES: C[C@@]1(O)COCCN(c2nc(OC[C@@]34CCCN3C[C@H](F)C4)nc3sc4c(Cl)nncc4c23)C1] Int-162 | 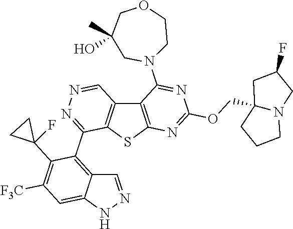 [SMILES: C[C@@]1(O)COCCN(c2nc(OC[C@@]34CCCN3C[C@H](F)C4)nc3sc4c(-c5c(C6(F)CC6)c(C(F)(F)F)cc6[nH]ncc56)nncc4c23)C1] | (6S)-4-(8-(5-(1- fluorocyclopropyl)-6- (trifluoromethyl)-1H- indazol-4-yl)-2- (((2R,7aS)-2- fluorotetrahydro-1H- pyrrolizin-7a(5H)- yl)methoxy)pyrimido [5′,4′:4,5]thieno[2,3- d]pyridazin-4-yl)-6- methyl-1,4-oxazepan-6-ol | 717 |
| Ex- 284 | 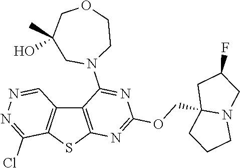 [SMILES: C[C@@]1(O)COCCN(c2nc(OC[C@@]34CCCN3C[C@H](F)C4)nc3sc4c(Cl)nncc4c23)C1] Int-32 | 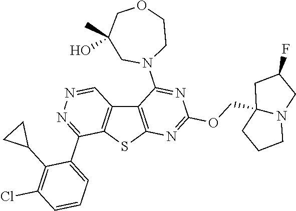 [SMILES: C[C@@]1(O)COCCN(c2nc(OC[C@@]34CCCN3C[C@H](F)C4)nc3sc4c(-c5cccc(Cl)c5C5CC5)nncc4c23)C1] | (S)-4-(8-(3-chloro-2- cyclopropylphenyl)-2- (((2R,7aS)-2- fluorotetrahydro-1H- pyrrolizin-7a(5H)- yl)methoxy)pyrimi- do[5′,4′:4,5]thieno[2,3- dipyridazin-4-yl)-6- methyl-1,4-oxazepan-6-ol | 625 |
| Ex- 285 | 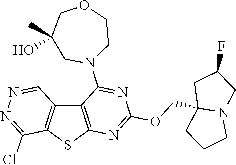 [SMILES: C[C@@]1(O)COCCN(c2nc(OC[C@@]34CCCN3C[C@H](F)C4)nc3sc4c(Cl)nncc4c23)C1] Int-32 | 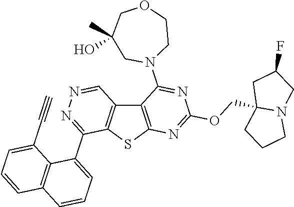 [SMILES: C#Cc1cccc2cccc(-c3nncc4c3sc3nc(OC[C@@]56CCCN5C[C@H](F)C6)nc(N5CCOC[C@@](C)(O)C5)c34)c12] | (S)-4-(8-(8- ethynylnaphthalen-1-yl)- 2-(((2R,7aS)-2- fluorotetrahydro-1H- pyrrolizin-7a(5H)- yl)methoxy)pyrimi- do[5′,4′:4,5]thieno[2,3- dipyridazin-4-yl)-6- methyl-1,4-oxazepan-6-ol | 625 |
| Ex- 286 | 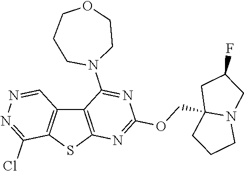 [SMILES: F[C@H]1CN2CCC[C@@]2(COc2nc(N3CCCOCC3)c3c(n2)sc2c(Cl)nncc23)C1] Int-164 | 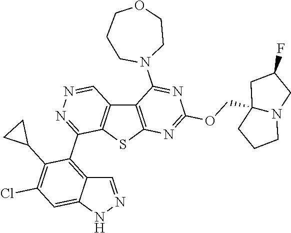 [SMILES: F[C@H]1CN2CCC[C@@]2(COc2nc(N3CCCOCC3)c3c(n2)sc2c(-c4c(C5CC5)c(Cl)cc5[nH]ncc45)nncc23)C1] | 4-(8-(6-chloro-5- cyclopropyl-1H-indazol- 4-yl-2-(((2R,7aS)-2- fluorotetrahydro-1H- pyrrolizin-7a(5H)- yl)methoxy)pyrimi- do[5′,4:′4,5]thieno[2,3- dipyridazin-4-yl)-1,4- oxazepane | 635 |
| Ex- 287 | 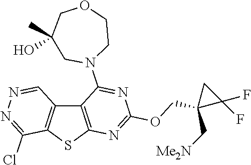 [SMILES: CN(C)C[C@@]1(COc2nc(N3CCOC[C@@](C)(O)C3)c3c(n2)sc2c(Cl)nncc23)CC1(F)F] Int-163 | 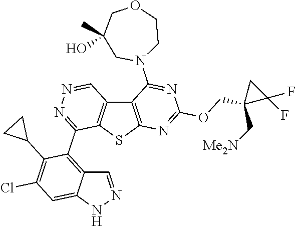 [SMILES: CN(C)C[C@@]1(COc2nc(N3CCOC[C@@](C)(O)C3)c3c(n2)sc2c(-c4c(C5CC5)c(Cl)cc5[nH]ncc45)nncc23)CC1(F)F] | (6S)-4-(8-(6-chloro-5- cyclopropyl-1H-indazol- 4-yl)-2-(((R)-1- ((dimethylamino)methyl)- 2,2- difluorocyclopropyl)meth- oxy)pyrimido[5′,4′:4,5]thi- eno[2,3-d]pyridazin-4-yl)- 6-methyl-1,4-oxazepan-6- ol | 671 |

### Example 288: (6S)-4-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]selenopheno[2,3-d]pyrimidin-4-yl)-6-methyl-1,4-oxazepan-6-ol (Ex-288)

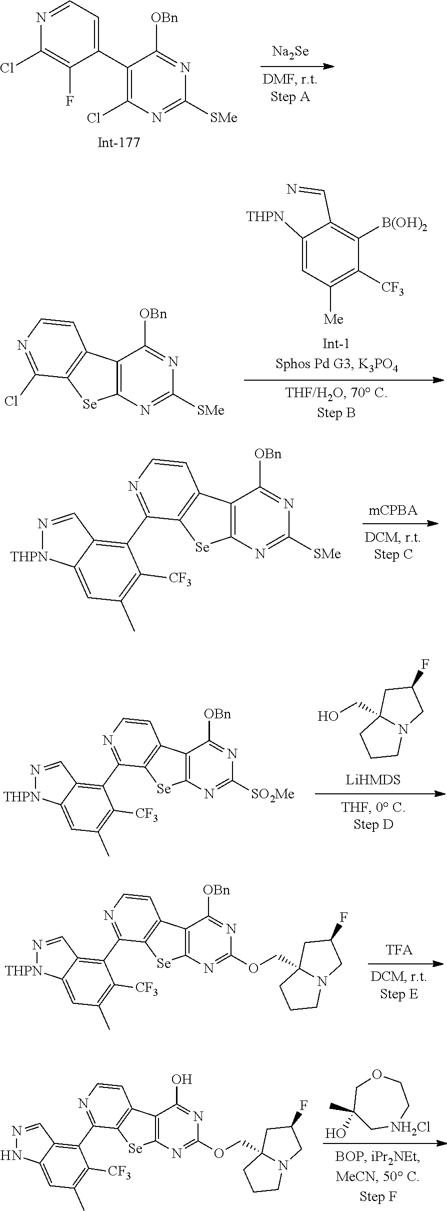

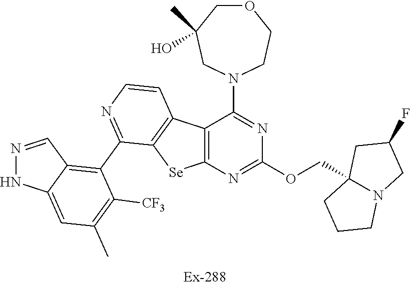 [SMILES: Cc1cc2[nH]ncc2c(-c2nccc3c2[se]c2nc(OC[C@@]45CCCN4C[C@H](F)C5)nc(N4CCOC[C@@](C)(O)C4)c23)c1C(F)(F)F]

### Step A: 4-(benzyloxy)-8-chloro-2-(methylthio)pyrido[4′,3′:4,5]selenopheno[2,3-d]pyrimidine

In a glovebox, 4-(benzyloxy)-6-chloro-5-(2-chloro-3-fluoropyridin-4-yl)-2-(methylthio)pyrimidine (Int-177) (1.00 g, 2.52 mmol), DMF (20.0 mL), and sodium selenide (0.631 g, 5.05 mmol) were combined. The mixture was stirred under an inert atmosphere for 2 h before being diluted with DCM and water. The layers were shaken and separated and the aqueous phase was extracted DCM. The combined extracts were dried over Na₂SO₄, filtered, and concentrated. The crude reaction mixture was purified by flash column chromatography 0 to 40% EtOAc in hexanes) to provide 4-(benzyloxy)-8-chloro-2-(methylthio)pyrido[4′,3′:4,5]selenopheno[2,3-d]pyrimidine. MS (ESI): m/z (M+H)⁺ 422.

### Step B 4-(benzyloxy)-8-(6-methyl-1-(tetrahydro-2H-pyran-2-yl)-5-(trifluoromethyl)-1H-indazol-4-yl)-2-(methylthio)pyrido[4′,3′:4,5]selenopheno[2,3-d]pyrimidine

4-(benzyloxy)-8-chloro-2-(methylthio)pyrido[4′,3′:4,5]selenopheno[2,3-d]pyrimidine (200 mg, 0.475 mmol), (6-methyl-1-(tetrahydro-2H-pyran-2-yl)-5-(trifluoromethyl)-1H-indazol-4-yl)boronic acid (Int-1) (187 mg, 0.570 mmol), SPhos Pd G3 (37 mg, 0.048 mmol), and THF (3.17 mL) were combined under an inert atmosphere before K₃PO₄ (1.19 mL, 2.38 mmol, 2 M in H₂O) was added. The mixture was then stirred at 70° C. for 13 h. The mixture was cooled to room temperature, diluted with brine (20 mL), and extracted with ethyl acetate (3×20 mL). The combined extracts were dried over Na₂SO₄, filtered, and concentrated in vacuo. The crude residue was purified by flash-column chromatography (0 to 50% EtOAc in hexanes) to provide 4-(benzyloxy)-8-(6-methyl-1-(tetrahydro-2H-pyran-2-yl)-5-(trifluoromethyl)-1H-indazol-4-yl)-2-(methylthio)pyrido[4′,3′:4,5]selenopheno[2,3-d]pyrimidine. MS (ESI): m/z (M+H)⁺ 670.

### Step C: 4-(benzyloxy)-8-(6-methyl-1-(tetrahydro-2H-pyran-2-yl)-5-(trifluoromethyl)-1H-indazol-4-yl)-2-(methylsulfonyl)pyrido[4′,3′:4,5]selenopheno[2,3-d]pyrimidine

mCPBA (178 mg, 0.721 mmol, 70% w/w in H₂O) was added to a solution of 4-(benzyloxy)-8-(6-methyl-1-(tetrahydro-2H-pyran-2-yl)-5-(trifluoromethyl)-1H-indazol-4-yl)-2-(methylthio)pyrido[4′,3′:4,5]selenopheno[2,3-d]pyrimidine (241 mg, 0.360 mmol) in dichloromethane (2.40 mL) at room temperature. The mixture was stirred for 10 min before being purified via silica gel chromatography (0 to 60% (3:1 EtOAc:EtOH) in hexanes) to provide 4-(benzyloxy)-8-(6-methyl-1-(tetrahydro-2H-pyran-2-yl)-5-(trifluoromethyl)-1H-indazol-4-yl)-2-(methylsulfonyl)pyrido[4′,3′:4,5]selenopheno[2,3-d]pyrimidine. MS (ESI): m/z (M+H)⁺ 702.

### Step D: 4-(benzyloxy)-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]selenopheno[2,3-d]pyrimidine

LiHMDS (0.255 mL, 0.255 mmol, 1M in THF) was added dropwise to a solution of ((2R,7aS)-2-fluorotetrahydro-1H-pyrrolizin-7a(5H)-yl) methanol (54.1 mg, 0.340 mmol) in THF (1.70 mL) at room temperature. After 15 min, this solution was chilled to 0° C. and 4-(benzyloxy)-8-(6-methyl-1-(tetrahydro-2H-pyran-2-yl)-5-(trifluoromethyl)-1H-indazol-4-yl)-2-(methylsulfonyl)pyrido[4′,3′:4,5]selenopheno[2,3-d]pyrimidine (119 mg, 0.170 mmol) was added. The mixture was warmed to room temperature and stirring was continued for 15 min, at which time the mixture was diluted with brine (10 mL) and sat. aq. NaHCO₃ (10 mL). The diluted mixture was extracted with ethyl acetate (3×20 mL) and the combined extracts were dried over Na₂SO₄, filtered, and concentrated in vacuo to provide 4-(benzyloxy)-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]selenopheno[2,3-d]pyrimidine, which was used without further purification. MS (ESI): m/z (M+H)⁺ 781.

### Step E: 2-(((2R,7aS)-2-fluorotetrahydro-1H-pyrrolizin-7a(5H)-yl)methoxy)-8-(16-methyl-5-(trifluoromethyl)-1H-indazol-4-yl)pyrido[4′,3′:4,5]selenopheno[2,3-d]pyrimidin-4-01

4-(benzyloxy)-2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]selenopheno[2,3-d]pyrimidine (128 mg, 0.164 mmol) was dissolved in a mixture of DCM (0.821 mL) and TFA (0.821 mL). The solution was stirred at room temperature for 2.5 h before it was transferred dropwise to a solution of sat. aq. NH₄HCO₃ (30 ml). The resulting mixture was extracted with 30% v/v/PrOH in CHCl₃ (3×15 mL). The combined extracts were dried by passage through a phase-separation column, then dried further over Na₂SO₄, filtered, and concentrated in vacuo to provide 2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]selenopheno[2,3-d]pyrimidin-4-ol, which was used without further purification. MS (ESI): m/z (M+H)⁺ 607.

### Step F (6S)-4-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]selenopheno[2,3-d]pyrimidin-4-yl)-6-methyl-1,4-oxazepan-6-ol (Ex-288)

2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]selenopheno[2,3-d]pyrimidin-4-ol (99 mg, 0.16 mmol) was dissolved in MeCN (1.6 mL). BOP (360 mg, 0.82 mmol) was added, and the mixture was stirred at room temperature for 30 min. Next, N,N-diisopropylethylamine (0.43 mL, 2.5 mmol) and(S)-6-methyl-1,4-oxazepan-6-ol hydrochloride (140 mg, 0.82 mmol) were added, and the mixture was heated with stirring to 50° C. After 75 min, the mixture was cooled to room temperature and diluted with MeCN (6.0 mL), and the diluted mixture was filtered. The filtrate was purified by preparative reverse-phase HPLC (MeCN/H₂O w/TFA modifier). The liquid fractions containing product were basified with the addition of sat. aq. NaHCO₃ to attain pH >8 before they were extracted with 30% v/v iPrOH in CHCl₃. The combined extracts were dried over Na₂SO₄, filtered, and concentrated to provide (6S)-4-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]selenopheno[2,3-d]pyrimidin-4-yl)-6-methyl-1,4-oxazepan-6-ol (Ex-288). MS (ESI): m/z (M+H)⁺ 720. ¹H NMR (500 MHz, CD: OD) δ 8.71 (d, J=4.5 Hz, 1H), 7.86 (d, J=5.7 Hz, 1H), 7.73 (s, 1H), 7.48 (d, J=16.5 Hz, 1H), 5.29 (d, J=53.6 Hz, 1H), 4.29 (d, J=10.5 Hz, 1H), 4.20-4.17 (m, 1H), 4.09-3.97 (3H), 3.91-3.84 (m, 2H), 3.70 (dd, J=12.5, 6.7 Hz, 1H), 3.47 (dd, J=12.5, 6.7 Hz, 1H), 3.24 (br s, 2H), 3.17 (brs, 1H), 3.03-2.98 (m, 1H), 2.73 (s, 3H), 2.31-2.15 (m, 2H), 2.11-2.07 (m, 1H), 2.01-1.95 (m, 2H), 1.91-1.85 (m, 1H), 1.29 (br s, 1H), 1.19 (d, J=15.3 Hz, 3H).

The example in the table below was synthesized using a similar procedure as described in the synthesis of Ex-288 by making the appropriate substitutions for starting material, intermediates, and or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Ex. | Material | Structure | Compound Name | Found |
| Ex- 289 | 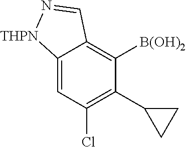 Int-33 | 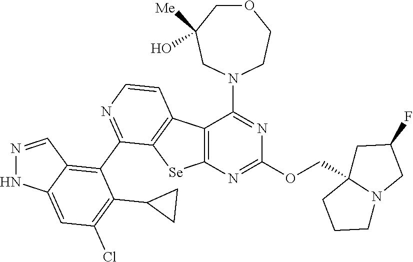 [SMILES: C[C@@]1(O)COCCN(c2nc(OC[C@@]34CCCN3C[C@H](F)C4)nc3[se]c4c(-c5c(C6CC6)c(Cl)cc6[nH]ncc56)nccc4c23)C1] | (6S)-4-(8-(6-chloro-5- cyclopropyl-1H-indazol- 4-yl)-2-(((2R,7aS)-2- fluorotetrahydro-1H- pyrrolizin-7a(5H)- yl)methoxy)pyri- do[4′,3′:4,5]seleno- pheno[2,3- d]pyrimidin-4-yl)-6- methyl-1,4-oxazepan-6-ol | 712 |

### Example 290: (6S)-4-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]furo[2,3-d]pyrimidin-4-yl)-6-methyl-1,4-oxazepan-6-ol (Ex-290)

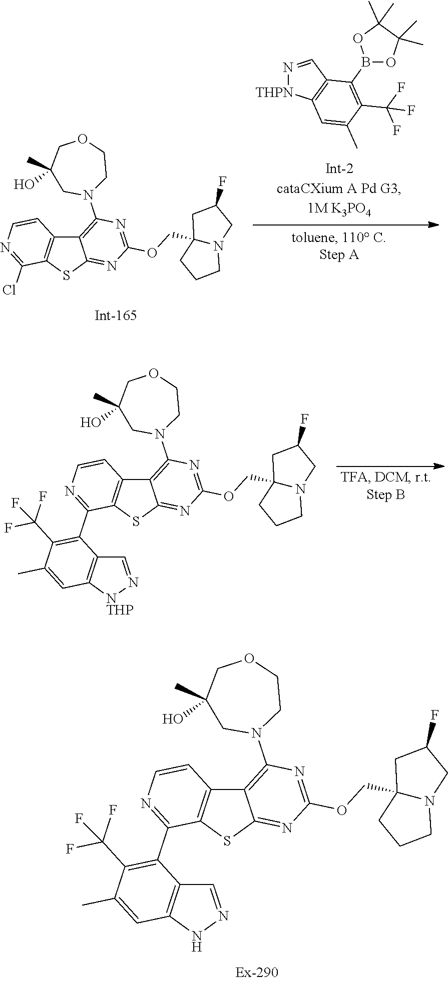

### Step A: (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]furo[2,3-d]pyrimidin-4-yl)-6-methyl-1,4-oxazepan-6-ol

A mixture of(S)-4-(8-chloro-2-(((2R,7aS)-2-fluorotetrahydro-1H-pyrrolizin-7a(5H)-yl)methoxy)pyrido[4′,3′:4,5]furo[2,3-d]pyrimidin-4-yl)-6-methyl-1,4-oxazepan-6-ol (Int-165) (32 mg, 0.065 mmol), 6-methyl-1-(tetrahydro-2H-pyran-2-yl)-4-(4,4,5,5-tetramethyl-1,3,2-dioxaborolan-2-yl)-5-(trifluoromethyl)-1H-indazole (Int-2) (40 mg, 0.098 mmol), cataCXium A Pd G3 (24 mg, 0.032 mmol) and IM aq. K₃PO₄ (0.32 mL, 0.325 mmol) in toluene (1.2 ml) was degassed and nitrogen charged (3 times). Then the mixture was stirred at 110° C. under nitrogen atmosphere for 5 h. After cooling, the mixture was partitioned between EtOAc and water. The organic phase was washed with brine, dried over sodium sulfate, filtered, and concentrated. The crude residue was purified via silica gel chromatography (0% to 40% MeOH in EtOAc) to give (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]furo[2,3-d]pyrimidin-4-yl)-6-methyl-1,4-oxazepan-6-ol. MS (ESI): m/z (M+H)⁺ 740.

### Step B: (6S)-4-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]furo[2,3-d]pyrimidin-4-yl)-6-methyl-1,4-oxazepan-6-ol (Ex-290)

To a stirred solution of (6S)-4-(2-(((2R,7aS)-2-fluorotetrahydro-1H-pyrrolizin-7a(5H)-yl)methoxy)-8-(6-methyl-1-(tetrahydro-2H-pyran-2-yl)-5-(trifluoromethyl)-1H-indazol-4-yl)pyrido[4′,3′:4,5]furo[2,3-d]pyrimidin-4-yl)-6-methyl-1,4-oxazepan-6-ol (150 mg, 0.199 mmol) in DCM (1.2 ml) was added TFA (500 μl) and the reaction was allowed to stir at r.t. for 1 h. After 1 h, the reaction was concentrated in vacuo. The residue was partitioned between sat. aq. sodium bicarbonate and chloroform. The organic phase was washed with brine, dried over sodium sulfate, filtered, and concentrated. The crude residue was purified via silica gel chromatography (0 to 25% MeOH in CHCl₃) to give (6S)-4-(2-(((2R,7aS)-2-fluorotetrahydro-1H-pyrrolizin-7a(5H)-yl)methoxy)-8-(6-methyl-5-(trifluoromethyl)-1H-indazol-4-yl)pyrido[4′,3′:4,5]furo[2,3-d]pyrimidin-4-yl)-6-methyl-1,4-oxazepan-6-ol (Ex-290), MS (ESI): (M+H)⁺ 656. ¹H NMR (400 MHz, CDCl₃) δ=10.27-10.05 (m, 1H), 8.72-8.67 (m, 1H), 7.72-7.65 (m, 1H), 7.56-7.50 (m, 2H), 5.86-5.69 (m, 1H), 5.43-5.11 (m, 1H), 4.55 (br dd, J=2.7, 13.4 Hz, 1H), 4.37 (dd, J=5.8, 14.8 Hz, 1H), 4.23-4.05 (m, 3H), 3.92-3.70 (m, 3H), 3.59 (dd, J=1.8, 14.8 Hz, 1H), 3.44 (d, J=12.1 Hz, 1H), 3.30-3.19 (m, 2H), 3.15-3.12 (m, 1H), 3.01-2.90 (m, 1H), 2.74 (s, 3H), 2.21-2.16 (m, 1H), 2.13-2.09 (m, 1H), 2.03-1.80 (m, 4H), 1.39 (s, 3H).

The examples in the table below were synthesized using a similar procedure as described in the synthesis of Ex-290 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | | [M + H]⁺ |
|---|---|---|---|---|
| Ex. | Starting Material | Structure | Compound Name | Found |
| Ex- 291 | 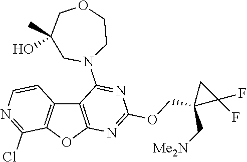 [SMILES: CN(C)C[C@@]1(COc2nc(N3CCOC[C@@](C)(O)C3)c3c(n2)oc2c(Cl)nccc23)CC1(F)F] Int-166 | 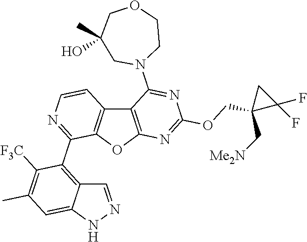 [SMILES: Cc1cc2[nH]ncc2c(-c2nccc3c2oc2nc(OC[C@]4(CN(C)C)CC4(F)F)nc(N4CCOC[C@@](C)(O)C4)c23)c1C(F)(F)F] | (6S)-4-(2-(((R)-1- ((dimethylamino)methyl)- 2.2- difluorocyclopropyl)meth- oxy)-8-(6-methyl-5- (trifluoromethyl)-1H- indazol-4-yl)pyri do[4′,3′:4,5]furo[2,3- d]pyrimidin-4-yl)-6- methyl-1,4-oxazepan-6-ol | 662 |
| Ex- 292 | 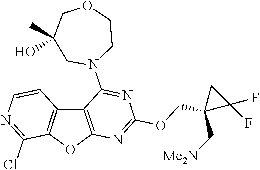 [SMILES: CN(C)C[C@@]1(COc2nc(N3CCOC[C@@](C)(O)C3)c3c(n2)oc2c(Cl)nccc23)CC1(F)F] Int-166 | 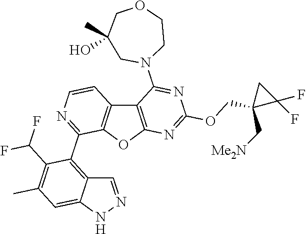 [SMILES: Cc1cc2[nH]ncc2c(-c2nccc3c2oc2nc(OC[C@]4(CN(C)C)CC4(F)F)nc(N4CCOC[C@@](C)(O)C4)c23)c1C(F)F] | (6S)-4-(8-(5- (difluoromethyl)-6- methyl-1H-indazol-4-yl)- 2-(((R)-1- ((dimethylamino)methyl)- 2,2- difluorocyclopropyl)meth- oxy)pyrido[4′,3′:4,5]furo[2,3- d]pyrimidin-4-yl)-6- methyl-1,4-oxazepan-6-ol | 644 |
| Ex- 293 | 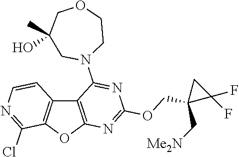 [SMILES: CN(C)C[C@@]1(COc2nc(N3CCOC[C@@](C)(O)C3)c3c(n2)oc2c(Cl)nccc23)CC1(F)F] Int-166 | 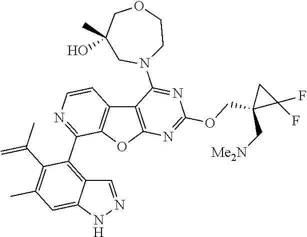 [SMILES: C=C(C)c1c(C)cc2[nH]ncc2c1-c1nccc2c1oc1nc(OC[C@]3(CN(C)C)CC3(F)F)nc(N3CCOC[C@@](C)(O)C3)c12] | (6S)-4-(2-(((R)-1- ((dimethylamino)methyl)- 2,2- difluorocyclopropyl)meth- oxy)-8-(6-methyl-5-(prop- 1-en-2-yl)-1H-indazol-4- yl)pyrido[4′,3′:4,5]furo[2,3- d]pyrimidin-4-yl)-6- methyl-1,4-oxazepan-6-ol | 634 |
| Ex- 294 | 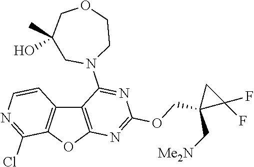 [SMILES: CN(C)C[C@@]1(COc2nc(N3CCOC[C@@](C)(O)C3)c3c(n2)oc2c(Cl)nccc23)CC1(F)F] Int-166 | 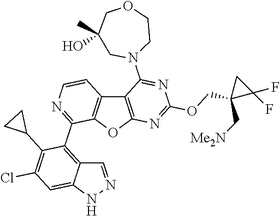 [SMILES: CN(C)C[C@@]1(COc2nc(N3CCOC[C@@](C)(O)C3)c3c(n2)oc2c(-c4c(C5CC5)c(Cl)cc5[nH]ncc45)nccc23)CC1(F)F] | (6S)-4-(8-(6-chloro-5- cyclopropyl-1H-indazol- 4-yl)-2-(((R)-1- ((dimethylamino)methyl)- 2,2- difluorocyclopropyl)meth- oxy)pyrido[4′,3′:4,5]furo[2,3- d]pyrimidin-4-yl)-6- methyl-1,4-oxazepan-6-ol | 654 |

### Example 295: (S)-4-(19-(5-cyclopropyl-6-methyl-1H-indazol-4-yl)-2-(2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepan-6-ol (Ex-295)

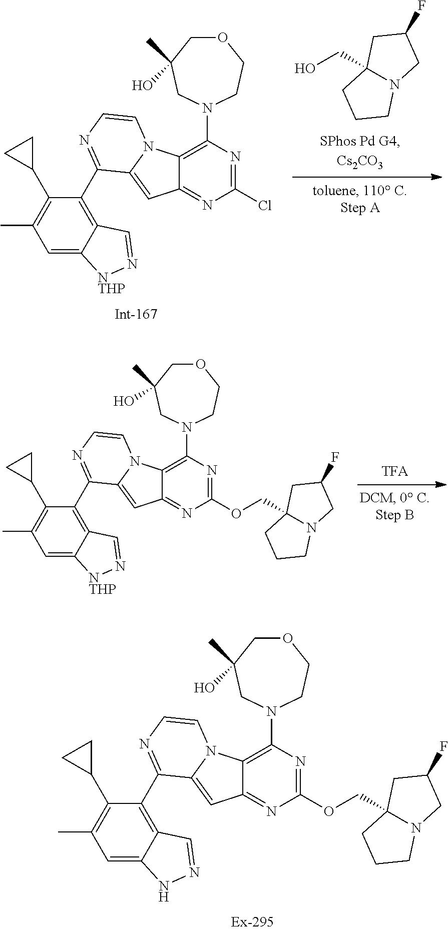

### Step A: (6S)-4-(9-(5-cyclopropyl-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrazino[1′,2′: 1,5]pyrrolo[3,2-d]pyrimidin-4-yl)-6-methyl-1,4-oxazepan-6-ol

A round bottom flask with stir bar was charged with (6S)-4-(2-chloro-9-(5-cyclopropyl-6-methyl-1-(tetrahydro-2H-pyran-2-yl)-1H-indazol-4-yl)pyrazino[1′,2′:1,5]pyrrolo[3,2-d]pyrimidin-4-yl)-6-methyl-1,4-oxazepan-6-ol (Int-167) (115 mg, 0.196 mmol), SPhos Pd G4 (21.6 mg, 0.027 mmol), Cs₂CO₃ (221.2 mg, 0,679 mmol), ((2R,7aS)-2-fluorotetrahydro-1H-pyrrolizin-7a(5H)-yl) methanol (32.4 mg, 0.204 mmol) and toluene (2.7 mL). The mixture was degassed with N₂ (3×) and stirred at 110° C. under N₂ for 2 h. After cooling, the mixture was diluted with AcOEt and water. The mixture was extracted with AcOEt (3×) and the combined organic layers were washed with brine, dried over Na₂SO₄, filtered, and concentrated. The residue was purified via silica gel chromatography (0 to 20% MeOH in CHCl₃) to afford (6S)-4-(9-(5-cyclopropyl-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepan-6-ol. MS (ESI): m/z (M+H) 711. ¹H NMR (400 MHz, CDCl₃) δ 8.06-7.97 (m, 2H), 7.73-7.52 (m, 2H), 7.08-7.06 (m, 1H), 6.59-6.57 (m, 1H), 5.74-5.69 (m, 1H), 5.34-5.21 (m, 1H), 4.21-3.98 (m, 5H), 3.86-3.71 (m, 5H), 3.65-3.61 (m, 1H), 2.44-3.33 (m, 2H), 3.26-3.21 (m, 1H), 3.14-3.12 (m, 1H), 2.99-2.92 (m, 1H), 2.67-2.66 (m, 3H), 2.58-2.46 (m, 1H), 2.20-2.12 (m, 3H), 2.08-2.03 (m, 2H), 1.99-1.82 (m, 4H), 1.80-1.73 (m, 3H), 1.41-1.38 (m, 3H), 0.89-0.85 (m, 0.5H), 0.80-0.75 (m, 0.5H), 0.65-0.60 (m, 0.5H), 0.45-0.38 (m, 0.5H), 0.21-0.13 (m, 2H).

### Step B: (S)-4-(9-(5-cyclopropyl-6-methyl-1H-indazol-4-yl)-2-(((2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepan-6-ol (Ex-295)

To a stirred solution of (6S)-4-(9-(5-cyclopropyl-6-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepan-6-ol (44.7 mg, 0.0629 mmol) in DCM (0.63 mL) at 0° C. was added TFA (0.21 mL) and the reaction was allowed to stir at 0° C. for 30 min. The reaction was quenched with sat. aq. NaHCO₃ and extracted with AcOEt (3×). The combined organic layers were washed with brine, dried over Na₂SO₄, filtered, and concentrated. The residue was purified by preparative reverse-phase HPLC (MeCN/H₂O w/formic acid modifier) to afford(S)-4-(9-(5-cyclopropyl-6-methyl-1H-indazol-4-yl)-2-(((2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepan-6-ol (Ex-295). MS (ESI): m/z (M+H)⁺ 627. ¹H NMR (400 MHz, CDCl₃) δ 10.51 (br, 1H), 8.07-8.03 (m, 1H), 8.04-7.98 (m, 1H), 7.78-7.68 (m, 1H), 7.43-7.42 (m, 1H), 7.08 (br, 1H), 6.59 (s, 1H), 5.34-5.20 (m, 1H), 4.28-4.19 (m, 1H), 4.15-4.09 (m, 1H), 4.04-3.98 (m, 2H), 3.85-3.78 (m, 3H), 3.65-3.61 (m, 1H), 3.45-3.35 (m, 2H), 3.27-3.15 (m, 3H), 2.98-2.84 (m, 1H), 2.63-2.62 (m, 3H), 2.14-2.03 (m, 4H), 1.98-1.88 (m, 3H), 1.41-1.40 (m, 3H), 0.80-0.75 (m, 0.5H), 0.64-0.60 (m, 0.5H), 0.46-0.42 (m, 0.5H), 0.21-0.13 (m, 2H), −0.1-−0.08 (m, 0.5H).

The examples in the table below were synthesized using a similar procedure as described in the synthesis of Ex-295 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | | SFC/ | |
|---|---|---|---|---|---|
| | | | | HPLC | [M + |
| | Starting | | Compound | separation | H]⁺ |
| Ex. | Material | Structure | Name | conditions | Found |
| Ex- 296 | 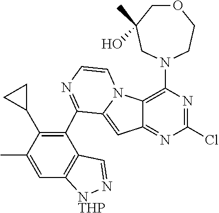 Int-167 | 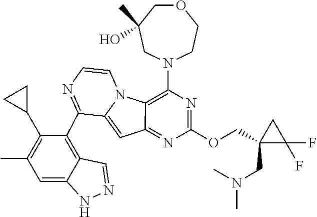 [SMILES: Cc1cc2[nH]ncc2c(-c2nccn3c2cc2nc(OC[C@]4(CN(C)C)CC4(F)F)nc(N4CCOC[C@@](C)(O)C4)c23)c1C1CC1] | (S)-4-(9-(5- cyclopropyl-6- methyl-1H-indazol-4- yl)-2-(((R)-1- ((dimethylamino)meth- yl)-2,2- difluorocyclopropyl) methoxy)pyrazino [1′,2′:1,5]pyrrolo[3,2- d]pyrimidin-4-yl)-6- methyl-1,4-oxazepan- 6-ol | | 633 |
| Ex- 297 | 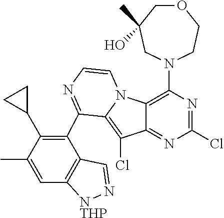 Int-170 | 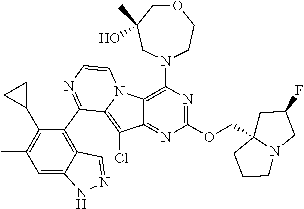 [SMILES: Cc1cc2[nH]ncc2c(-c2nccn3c2c(Cl)c2nc(OC[C@@]45CCCN4C[C@H](F)C5)nc(N4CCOC[C@@](C)(O)C4)c23)c1C1CC1] | (S)-4-(10-chloro-9-(5- cyclopropyl-6- methyl-1H-indazol-4- yl)-2-(((2R,7aS)-2- fluorotetrahydro-1H- pyrrolizin-7a(5H)- yl)methoxy)pyra- zino[1′,2′:1,5]pyrro- lo[3,2-d] pyrimidin-4-yl)-6- methyl-1,4-oxazepan- 6-ol | Column P, 40% EtOH in hexane w/0.1% Et₂NH, first eluting isomer | 661 |
| Ex- 298 | 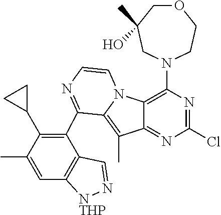 Int-173 | 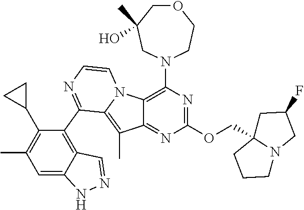 [SMILES: Cc1cc2[nH]ncc2c(-c2nccn3c2c(C)c2nc(OC[C@@]45CCCN4C[C@H](F)C5)nc(N4CCOC[C@@](C)(O)C4)c23)c1C1CC1] | (S)-4-(9-(5- cyclopropyl-6- methyl-1H-indazol-4- yl)-2-(((2R,7aS)-2- fluorotetrahydro-1H- pyrrolizin-7a(5H)- yl)methoxy)-10- methylpyrazi- no[1′,2′:1,5] pyrrolo[3,2- d]pyrimidin-4-yl)-6- methyl-1,4-oxazepan- 6-ol | | 641 |
| Ex- 299 | 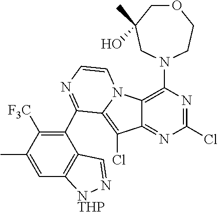 Int-171 | 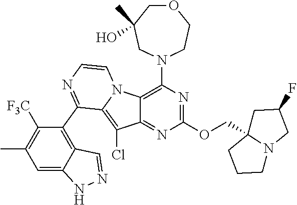 [SMILES: Cc1cc2[nH]ncc2c(-c2nccn3c2c(Cl)c2nc(OC[C@@]45CCCN4C[C@H](F)C5)nc(N4CCOC[C@@](C)(O)C4)c23)c1C(F)(F)F] | (S)-4-(10-chloro-2- (((2R,7aS)-2- fluorotetrahydro-1H- pyrrolizin-7a(5H)- yl)methoxy)-9-(6- methyl-5- (trifluoromethyl)-1H- indazol-4- yl)pyrazino[1′,2′:1,5] pyrrolo[3,2- d]pyrimidin-4-yl)-6- methyl-1,4-oxazepan- 6-ol | | 689 |

### Example 300: (S)-4-(10-chloro-9-(6-chloro-5-cyclopropyl-1H-indazol-4-yl)-2-(((2R,7aS)-2-fluorotetrahydro-1H-pyrrolizin-7a(5H)-yl)methoxy)pyrazino[1′2′:1,5]pyrrolo[3,2-d]pyrimidin-4-yl)-6-methyl-1,4-oxazepan-6-ol (Ex-300)

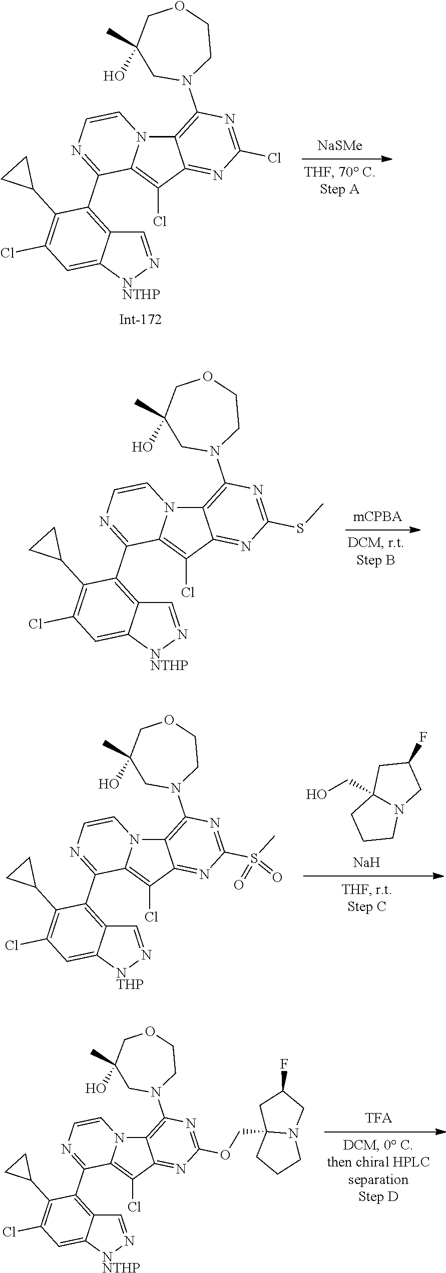

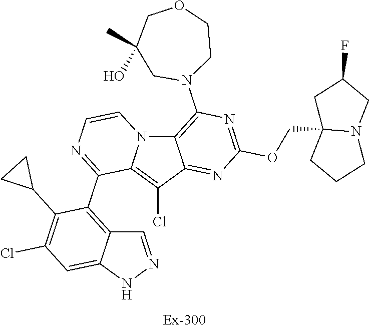 [SMILES: C[C@@]1(O)COCCN(c2nc(OC[C@@]34CCCN3C[C@H](F)C4)nc3c(Cl)c4c(-c5c(C6CC6)c(Cl)cc6[nH]ncc56)nccn4c23)C1]

### Step A: (6S)-4-(10-chloro-9-(6-chloro-5-cyclopropyl-1-(tetrahydro-2H-pyran-2-yl)-1H-indazol-4-yl)-2-(methylthio)pyrazino[1′,2′:1,5]pyrrolo[3,2-d]pyrimidin-4-yl)-6-methyl-1,4-oxazepan-6-ol

A round bottom flask with a stir bar was charged with (6S)-4-(2,10-dichloro-9-(6-chloro-5-cyclopropyl-1-(tetrahydro-2H-pyran-2-yl)-1H-indazol-4-yl)pyrazino[1′,2′:1,5]pyrrolo[3,2-d]pyrimidin-4-yl)-6-methyl-1,4-oxazepan-6-ol (Int-172) (90.9 mg, 0.141 mmol), THF (1.41 mL), and NaSMe (59.5 mg, 0.848 mmol). The reaction mixture was stirred at 70° C. for 50 min. The reaction mixture was cooled to room temperature and quenched by the addition of H₂O. The mixture was extracted with EtOAc (3×50 mL) and the combined organic layers were washed with brine (20 mL), dried over Na₂SO₄, filtered, and concentrated under reduced pressure. The residue was purified via silica gel chromatography (0 to 30% MeOH in EtOAc) to afford (6S)-4-(10-chloro-9-(6-chloro-5-cyclopropyl-1-(tetrahydro-2H-pyran-2-yl)-1H-indazol-4-yl)-2-(methylthio)pyrazino[1′,2′:1,5]pyrrolo[3,2-d]pyrimidin-4-yl)-6-methyl-1,4-oxazepan-6-ol. MS (ESI): m/z (M+H)⁺ 654. ¹H NMR (400 MHz, CDCl₃) δ 8.11-8.06 (m, 1H), 8.00-7.95 (m, 1H), 7.90-7.84 (m, 1H), 7.73-7.70 (m, 0.5H), 7.53-7.51 (m, 0.5H), 6.26-6.09 (m, 1H), 5.78-5.68 (m, 1H), 4.11-3.99 (m, 3H), 3.86-3.73 (m, 4H), 3.67-3.63 (m, 1H), 3.57-3.47 (m, 1H), 3.45-3.39 (m, 1H), 2.65 (s, 3H), 2.55-2.44 (m, 1H), 2.25-2.05 (m, 1H), 2.01-1.94 (m, 1H), 1.88-1.82 (m, 1H), 1.79-1.69 (m, 3H), 1.38 (s, 3H), 0.91-0.81 (m, 1H), 0.64-0.53 (m, 1H), 0.34-0.31 (m, 1H), 0.22-0.19 (m, 0.5H), 0.15-0.10 (m, 0.5H).

### Step B: (6S)-4-(10-chloro-9-(6-chloro-5-cyclopropyl-1-(tetrahydro-2H-pyran-2-yl)-1H-indazol-4-yl)-2-(methylsulfonyl)pyrazino[1′2′:1,5]pyrrolo[3,2-d]pyrimidin-4-yl)-6-methyl-1,4-oxazepam-6-ol

A round bottom flask with a stir bar was charged with (6S)-4-(10-chloro-9-(6-chloro-5-cyclopropyl-1-(tetrahydro-2H-pyran-2-yl)-1H-indazol-4-yl)-2-(methylthio)pyrazino[1′,2′:1,5]pyrrolo[3,2-d]pyrimidin-4-yl)-6-methyl-1,4-oxazepan-6-ol (75 mg, 0.114 mmol) and DCM (1.14 mL). The reaction mixture was cooled to 0° C. and mCPBA (66 mg, 0.285 mmol, 75 wt %) and the reaction mixture was stirred at room temperature for 20 min. The reaction mixture was quenched by the addition of sat. aq. Na₂S₂O₃ and sat. aq. NaHCO₃. The mixture was extracted with EtOAc (3×50 mL). The combined organic layer was washed with brine (20 mL), dried over dried over Na₂SO₄, filtered, and concentrated under reduced pressure to afford (6S)-4-(10-chloro-9-(6-chloro-5-cyclopropyl-1-(tetrahydro-2H-pyran-2-yl)-1H-indazol-4-yl)-2-(methylsulfonyl)pyrazino[1′,2′:1,5]pyrrolo[3,2-d]pyrimidin-4-yl)-6-methyl-1,4-oxazepan-6-ol, which was used in the next step without further purification. MS (ESI): m/z (M+H)⁺ 686, 688.

### Step C: (69)-4-(10-chloro-9-(6-chloro-5-cyclopropyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepan-6-ol

A round bottom flask with a stir bar was charged with (6S)-4-(10-chloro-9-(6-chloro-5-cyclopropyl-1-(tetrahydro-2H-pyran-2-yl)-1H-indazol-4-yl)-2-(methylsulfonyl)pyrazino[1′,2′:1,5]pyrrolo[3,2-d]pyrimidin-4-yl)-6-methyl-1,4-oxazepan-6-ol (78 mg, 0.114 mmol), ((2R,7aS)-2-fluorotetrahydro-1H-pyrrolizin-7a(5H)-yl) methanol (27.2 mg, 0.171 mmol) and THF (1.14 mL). To this mixture was added NaH (13.7 mg, 0.342 mmol, 60 wt % in mineral oil) and the reaction mixture was stirred at room temperature for 30 min. The reaction mixture was quenched by the addition of sat. aq. NaHCO₃ and the mixture was extracted with EtOAc (3×) and the combined organic layers were washed with brine, dried over Na₂SO₄, filtered, and concentrated under reduced pressure. The crude residue was purified via silica gel chromatography (0 to 80% MeOH in EtOAc) to afford (6S)-4-(10-chloro-9-(6-chloro-5-cyclopropyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepan-6-ol. MS (ESI): m/z (M+H)⁺ 765. ¹H NMR (400 MHz, CDCl₃) δ 8.07-8.02 (m, 1H), 7.99-7.93 (m, 1H), 7.89-7.83 (m, 1H), 7.73-7.70 (m, 0.5H), 7.50-7.48 (m, 0.5H), 6.80-6.68 (m, 1H), 5.78-5.67 (m, 1H), 5.32-5.13 (m, 1H), 4.31-4.27 (m, 1H), 4.19-4.15 (m, 1H), 4.07-3.99 (m, 3H), 3.85-3.73 (m, 4H), 3.65-3.61 (m, 1H), 3.45-3.33 (m, 2H), 3.25-3.13 (m, 3H), 2.98-2.92 (m, 1H), 2.54-2.43 (m, 1H), 2.21-2.07 (m, 5H), 1.97-1.68 (m, 7H), 1.35 (s, 3H), 0.88-0.80 (m, 0.5H), 0.61-0.56 (m, 1H), 0.38-0.33 (m, 1H), 0.20-0.17 (m, 0.5H), 0.12-0.07 (m, 0.5H), −0.01-−0.05 (m, 0.5H).

### Step D: (S)-4-(10-chloro-9-(6-chloro-5-cyclopropyl-1H-indazol-4-yl)-2-(((2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepan-6-ol (Ex-300)

To a stirred solution of (6S)-4-(10-chloro-9-(6-chloro-5-cyclopropyl-1-(tetrahydro-2H-pyran-2-yl)-1H-indazol-4-yl)-2-(((2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepan-6-ol (61.4 mg, 0.080 mmol) in DCM (0.90 mL) at 0° C. was added TFA (0.902 mL) and the reaction was allowed to stir at 0° C. After 25 min, the reaction was quenched with sat. aq. NaHCO₃ and extracted with EtOAc (3×). The combined organic layers were washed with brine, dried over Na₂SO₄, filtered, and concentrated under reduced pressure. The residue was purified via NH₂ silica gel chromatography (0 to 30% MeOH in EtOAc). The racemic(S)-4-(10-chloro-9-(6-chloro-5-cyclopropyl-1H-indazol-4-yl)-2-(((2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepan-6-ol was separated by preparative chiral HPLC (Column P, 40% EtOH in hexane w/0.1% Et₂NH) to give(S)-4-(10-chloro-9-(6-chloro-5-cyclopropyl-1H-indazol-4-yl)-2-(((2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepan-6-ol (Ex-300, the first eluting isomer from chiral HPLC). MS (ESI): m/z (M+H)⁺ 681, 683. ¹HNMR (400 MHz, CDCl₃) δ 10.41 (br, 1H), 8.08 (d, J=4.8 Hz, 1H), 7.99 (d, J=4.8 Hz, 1H), 7.69 (s, 1H), 7.55 (s, 1H), 6.69 (br, 1H), 5.33-5.19 (m, 1H), 4.31-4.29 (m, 1H), 4.20-4.18 (m, 1H), 4.06-3.99 (m, 2H), 3.85-3.73 (m, 3H), 3.65-3.62 (m, 1H), 3.45-3.38 (m, 2H), 3.28-3.21 (m, 2H), 3.14-3.13 (m, 1H), 2.99-2.93 (m, 1H), 2.22-2.08 (m, 3H), 2.00-1.86 (m, 4H), 1.40 (s, 3H), 0.66-0.55 (m, 2H), 0.41-0.35 (m, 1H), 0.21-0.18 (m, 1H).

The examples in the table below were synthesized using a similar procedure as described in the synthesis of Ex-300 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | | | | SFC/HPLC | |
|---|---|---|---|---|---|
| | | | | atropisomer | |
| | | | Compound | separation | [M + H]⁺ |
| Ex. | Starting Material | Structure | Name | conditions | Found |
| Ex- 301 | 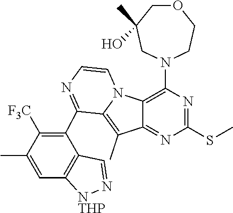 | 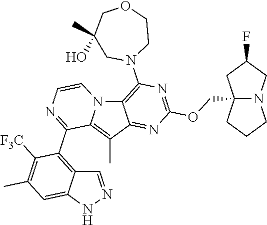 [SMILES: Cc1cc2[nH]ncc2c(-c2nccn3c2c(C)c2nc(OC[C@@]45CCCN4C[C@H](F)C5)nc(N4CCOC[C@@](C)(O)C4)c23)c1C(F)(F)F] | (S)-4-(2- (((2R,7aS)-2- fluorotetrahydro- 1H-pyrrolizin- 7a(5H)- yl)methoxy)-10- methyl-9-(6- methyl-5- (trifluoromethyl)- 1H-indazol-4- yl)pyrazino [1′,2′:1,5]pyrrolo [3,2-d]pyrimidin- 4-yl)-6-methyl-1,4- | Achiral preparative HPLC, MeCN/ Water w/ formic acid modifier, second eluting isomer | 669 |
| | Int-174 | | oxazepan-6-ol | | |
| Ex- 302 | 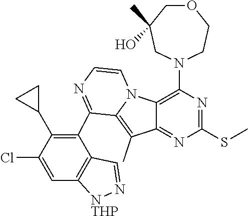 Int-175 | 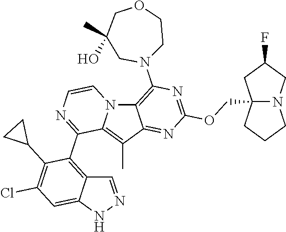 [SMILES: Cc1c2nc(OC[C@@]34CCCN3C[C@H](F)C4)nc(N3CCOC[C@@](C)(O)C3)c2n2ccnc(-c3c(C4CC4)c(Cl)cc4[nH]ncc34)c12] | (S)-4-(9-(6- chloro-5- cyclopropyl-1H- indazol-4-yl)-2- (((2R,7aS)-2- fluorotetrahydro- 1H-pyrrolizin- 7a(5H)- yl)methoxy)-10- methylpyrazino [1′,2′:1,5]pyrrolo [3,2-d]pyrimidin- 4-yl)-6-methyl- 1,4-oxazepan-6- ol | Achiral preparative HPLC, MeCN/ Water w/ formic acid modifier, second eluting isomer | 661 |
| Ex- 303 | 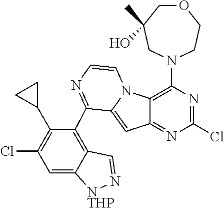 | 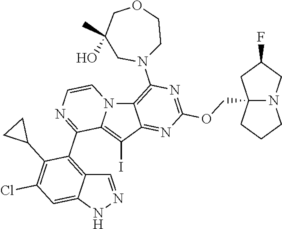 [SMILES: C[C@@]1(O)COCCN(c2nc(OC[C@@]34CCCN3C[C@H](F)C4)nc3c(I)c4c(-c5c(C6CC6)c(Cl)cc6[nH]ncc56)nccn4c23)C1] | (S)-4-(9-(6- chloro-5- cyclopropyl-1H- indazol-4-yl)-2- (((2R,7aS)-2- fluorotetrahydro- 1H-pyrrolizin- 7a(5H)- yl)methoxy)-10- iodopyrazino [1′,2′:1,5]pyrrolo [3,2-d]pyrimidin-4- yl)-6-methyl-1,4- oxazepan-6-ol | | 773, 775 |
| | Int-172 | | | | |

### Example 304: (S)-4-(9-(6-chloro-5-ethynyl-1H-indazol-4-yl)-10-fluoro-2-(((2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepan-6-ol (Ex-304)

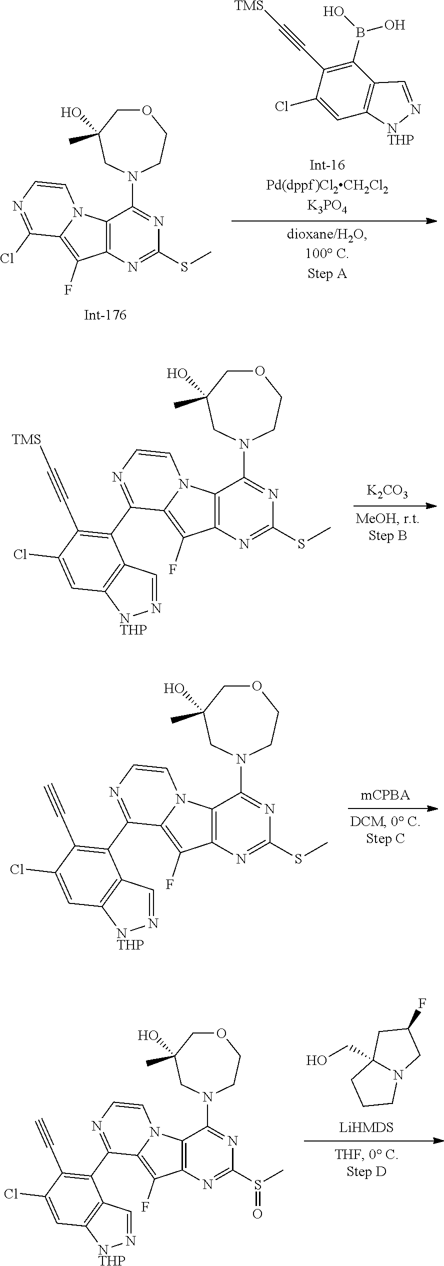

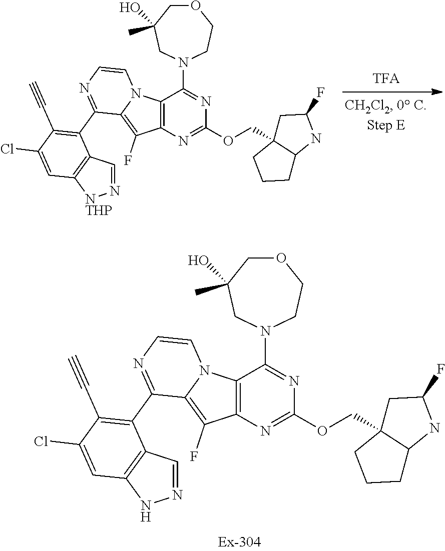

### Step A: (6S)-4-(9-(6-chloro-1-(tetrahydro-2H-pyran-2-yl)-5-((trimethylsilyl) ethynyl)-1H-indazol-4-yl)-10-fluoro-2-(methylthio)pyrazino[1′,2′:1,5]pyrrolo[3,2-d]pyrimidin-4-yl)-6-methyl-1,4-oxazepan-6-ol

K₃PO₄ (0.049 mL, 0.098 mmol, 2 M in H₂O) and Pd(dppf)Cl₂·CH₂Cl₂ (13 mg, 0.016 mmol) were added to a solution of (6-chloro-1-(tetrahydro-2H-pyran-2-yl)-5-((trimethylsilyl) ethynyl)-1H-indazol-4-yl)boronic acid (Int-16) (25 mg, 0.066 mmol) and(S)-4-(9-chloro-10-fluoro-2-(methylthio)pyrazino[1′,2′:1,5]pyrrolo[3,2-d]pyrimidin-4-yl)-6-methyl-1,4-oxazepan-6-ol (Int-176) (13 mg, 0.033 mmol) in 1,4-dioxane (1.0 mL). The reaction was heated to 100° C. and stirred for 2 h. The solution was cooled to room temperature and diluted with EtOAc. After washing with brine, the organic layer was dried over Na₂SO₄, filtered, and evaporated. The residue was purified by silica gel chromatography (hexane/EtOAc) to afford (6S)-4-(9-(6-chloro-1-(tetrahydro-2H-pyran-2-yl)-5-((trimethylsilyl) ethynyl)-1H-indazol-4-yl)-10-fluoro-2-(methylthio)pyrazino[1′,2′:1,5]pyrrolo[3,2-d]pyrimidin-4-yl)-6-methyl-1,4-oxazepan-6-ol. MS (ESI): m/z (M+H)⁺ 694.

### Step B: (6S)-4-(9-(6-chloro-5-ethynyl-1-(tetrahydro-2H-pyran-2-yl)-1H-indazol-4-yl)-10-fluoro-2-(methylthio)pyrazino[1′,2′:1,5]pyrrolo[3,2-d]pyrimidin-4-yl)-6-methyl-1,4-oxazepan-6-ol

K₂CO₃ (6.2 mg, 0.045 mmol) was added to a solution of (6S)-4-(9-(6-chloro-1-(tetrahydro-2H-pyran-2-yl)-5-((trimethylsilyl) ethynyl)-1H-indazol-4-yl)-10-fluoro-2-(methylthio)pyrazino[1′,2′:1,5]pyrrolo[3,2-d]pyrimidin-4-yl)-6-methyl-1,4-oxazepan-6-ol (6.8 mg, 0.0098 mmol) in MeOH (0.60 mL). After stirring at room temperature for 1 h, the solution was diluted with EtOAc and quenched with sat. aq. NH₄Cl. The layers were separated and the aqueous phase was extracted with EtOAc. The combined organic layers were washed with brine, dried over Na₂SO₄, filtered, and evaporated to obtain (6S)-4-(9-(6-chloro-5-ethynyl-1-(tetrahydro-2H-pyran-2-yl)-1H-indazol-4-yl)-10-fluoro-2-(methylthio)pyrazino[1′,2′:1,5]pyrrolo[3,2-d]pyrimidin-4-yl)-6-methyl-1,4-oxazepan-6-ol, which was used in the next step without further purification. MS (ESI): m/z (M+H)⁺ 622.

### Step C: (6S)-4-(9-(6-chloro-5-ethynyl-1-(tetrahydro-2H-pyran-2-yl)-1H-indazol-4-yl)-10-fluoro-2-(methylsulfinyl)pyrazino[1′,2′:1,5]pyrrolo[3,2-d]pyrimidin-4-yl)-6-methyl-1,4-oxazepan-6-ol

mCPBA (1.8 mg, 0.010 mmol) was added to (6S)-4-(9-(6-chloro-5-ethynyl-1-(tetrahydro-2H-pyran-2-yl)-1H-indazol-4-yl)-10-fluoro-2-(methylthio)pyrazino[1′,2′:1,5]pyrrolo[3,2-d]pyrimidin-4-yl)-6-methyl-1,4-oxazepan-6-ol (6.2 mg, 0.010 mmol) in DCM (0.20 mL) at 0° C. The reaction mixture was stirred at 0° C. for 1 h. The reaction mixture was quenched by the addition of aq. Na₂S₂O₃ and extracted with CHCl₃ (2×10 mL). The combined organic layers were washed with brine (15 mL), dried over Na₂SO₄, filtered and, concentrated under reduced pressure to obtain (6S)-4-(9-(6-chloro-5-ethynyl-1-(tetrahydro-2H-pyran-2-yl)-1H-indazol-4-yl)-10-fluoro-2-(methylsulfinyl)pyrazino[1′,2′:1,5]pyrrolo[3,2-d]pyrimidin-4-yl)-6-methyl-1,4-oxazepan-6-ol, which was used in the next step without further purification. MS (ESI): m/z (M+H)⁺ 638.

### Step D: (6S)-4-(19-16-chloro-5-ethynyl-1-(tetrahydro-2H-pyran-2-yl)-1H-indazol-4-yl)-10-fluoro-2-(((2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepan-6-ol

LiHMDS (0.034 mL, 0.034 mmol, 1 M in THF) was added to a solution of ((2R,7aS)-2-fluorotetrahydro-1H-pyrrolizin-7a(5H)-yl) methanol (4.2 mg, 0.026 mmol) in THF (0.20 mL) at 0° C. The reaction mixture was stirred at 0° C. for 30 min. A solution of (6S)-4-(9-(6-chloro-5-ethynyl-1-(tetrahydro-2H-pyran-2-yl)-1H-indazol-4-yl)-10-fluoro-2-(methylsulfinyl)pyrazino[1′,2′:1,5]pyrrolo[3,2-d]pyrimidin-4-yl)-6-methyl-1,4-oxazepan-6-ol (6.2 mg) in THF (0.20 mL) was added to the reaction. After stirring at 0° C. for 30 min, the mixture was diluted with EtOAc, washed with sat. aq. NH₄Cl and brine, then dried over Na₂SO₄, filtered, and evaporated. The residue was purified by silica gel chromatography (CHCl₃/EtOH) to afford (6S)-4-(9-(6-chloro-5-ethynyl-1-(tetrahydro-2H-pyran-2-yl)-1H-indazol-4-yl)-10-fluoro-2-(((2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepan-6-ol. MS (ESI): m/z (M+H)⁺ 733.

### Step E: (S)-4-(9-(6-chloro-5-ethynyl-1H-indazol-4-yl)-10-fluoro-2-(((2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepan-6-ol (Ex-304)

TFA (0.60 mL) was added to a solution of (6S)-4-(9-(6-chloro-5-ethynyl-1-(tetrahydro-2H-pyran-2-yl)-1H-indazol-4-yl)-10-fluoro-2-(((2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepan-6-ol (5.5 mg, 0.0075 mmol) in DCM (0.60 mL) at 0° C. After stirring at 0° C. for 3 h, the solution was diluted with CHCl₃ and quenched with sat. aq. NaHCO₃. The layers were separated and the aqueous phase was extracted with CHCl₃. The combined organic layers were washed with brine, dried over Na₂SO₄, filtered, and evaporated. The residue was purified by preparative reverse phase HPLC (MeCN/H₂O w/formic acid modifier). Sat. aq. NaHCO₃ was added to the purified fractions containing the title compound, followed by extraction with ethyl acetate. The extract was washed with brine and dried over Na₂SO₄. The mixture was filtered and the filtrate was evaporated under reduced pressure to afford(S)-4-(9-(6-chloro-5-ethynyl-1H-indazol-4-yl)-10-fluoro-2-(((2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepan-6-ol (Ex-304). MS (ESI): m/z (M+H)⁺ 649. ¹H NMR (400 MHz, CDCl₃) δ 7.98-7.92 (m, 1H), 7.89 (t, J=2.4 Hz, 1H), 7.78-7.76 (m, 1H), 7.53 (dd, J=5.4, 3.1 Hz, 1H), 5.36-5.22 (m, 1H), 4.32-4.17 (m, 2H), 4.07-3.99 (m, 2H), 3.84-3.76 (m, 3H), 3.64-3.60 (m, 1H), 3.43-3.13 (m, 6H), 2.99-2.95 (m, 1H), 2.33-2.22 (m, 1H), 2.19-2.12 (m, 2H), 2.03-1.88 (m, 5H), 1.42-1.40 (m, 3H).

### Example 305: (S)-4-(9-(6-chloro-5-cyclopropyl-1H-indazol-4-yl)-10-fluoro-2-(((2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepan-6-ol (Int-305)

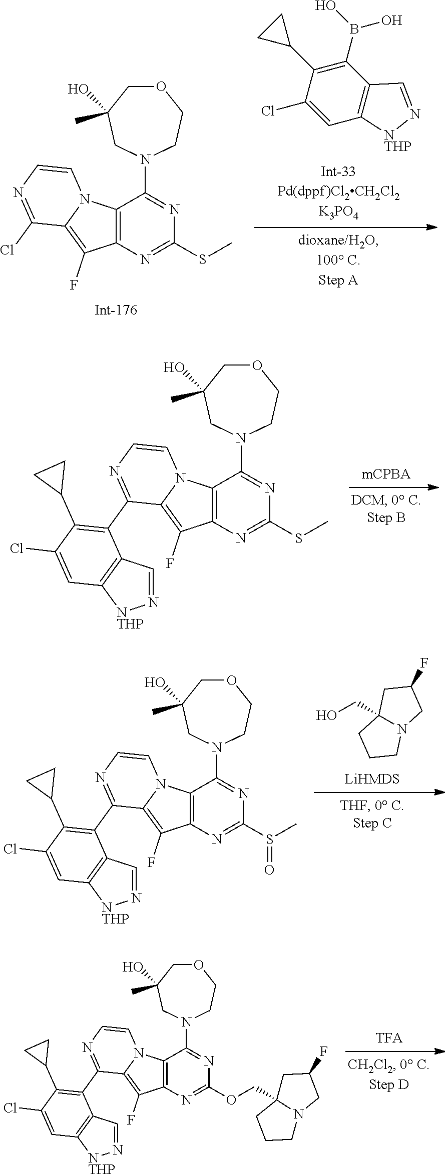

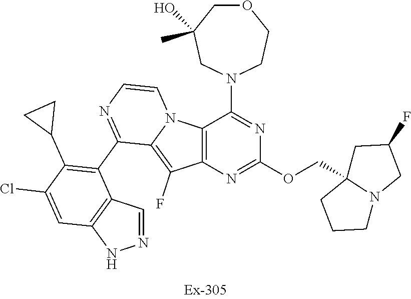 [SMILES: C[C@@]1(O)COCCN(c2nc(OC[C@@]34CCCN3C[C@H](F)C4)nc3c(F)c4c(-c5c(C6CC6)c(Cl)cc6[nH]ncc56)nccn4c23)C1]

### Step A: (6S)-4-(9-(6-chloro-5-cyclopropyl-1-(tetrahydro-2H-pyran-2-yl)-1H-indazol-4-yl)-10-fluoro-2-(methylthio)pyrazino[1′,2′:1,5]pyrrolo[3,2-d]pyrimidin-4-yl)-6-methyl-1,4-oxazepan-6-ol

K₃PO₄ (0.15 mL, 0.30 mmol, 2 M in H₂O) and Pd(dppf)Cl₂·CH₂Cl₂ (41 mg, 0.050 mmol) were added to a solution of(S)-4-(9-chloro-10-fluoro-2-(methylthio)pyrazino[1′,2′:1,5]pyrrolo[3,2-d]pyrimidin-4-yl)-6-methyl-1,4-oxazepan-6-ol (Int-176) (40 mg, 0.10 mmol) and (6-chloro-5-cyclopropyl-1-(tetrahydro-2H-pyran-2-yl)-1H-indazol-4-yl)boronic acid (Int-33) (58 mg, 0.18 mmol) in 1,4-dioxane (2.0 mL). The reaction was heated to 100° C. and stirred for 3 h. The solution was cooled at room temperature and diluted with EtOAc. After washing with brine, the organic layer was dried over Na₂SO₄, filtered, and evaporated. The residue was purified by silica gel chromatography (CHCl₃/EtOH) to afford (6S)-4-(9-(6-chloro-5-cyclopropyl-1-(tetrahydro-2H-pyran-2-yl)-1H-indazol-4-yl)-10-fluoro-2-(methylthio)pyrazino[1′,2′:1,5]pyrrolo[3,2-d]pyrimidin-4-yl)-6-methyl-1,4-oxazepan-6-ol. MS (ESI): m/z (M+H)⁺ 638.

### Step B: (6S)-4-(9-(6-chloro-5-cyclopropyl-1-(tetrahydro-2H-pyran-2-yl)-1H-indazol-4-yl)-10-fluoro-2-(methylsulfinyl)pyrazino[1′,2′:1,5]pyrrolo[3,2-d]pyrimidin-4-yl)-6-methyl-1,4-oxazepan-6-ol

mCPBA (14 mg, 0.079 mmol) was added to (6S)-4-(9-(6-chloro-5-cyclopropyl-1-(tetrahydro-2H-pyran-2-yl)-1H-indazol-4-yl)-10-fluoro-2-(methylthio)pyrazino[1,2′:1,5]pyrrolo[3,2-d]pyrimidin-4-yl)-6-methyl-1,4-oxazepan-6-ol (48 mg, 0.075 mmol) in DCM (0.75 mL) at 0° C. The reaction mixture was stirred at 0° C. for 1 h. The reaction mixture was quenched with sat. aq. Na₂S₂O₃ and extracted with CHCl₃ (2×10 mL). The combined organic layers were washed with brine (15 mL), dried over sodium sulfate, filtered, and concentrated under reduced pressure to obtain (6S)-4-(9-(6-chloro-5-cyclopropyl-1-(tetrahydro-2H-pyran-2-yl)-1H-indazol-4-yl)-10-fluoro-2-(methylsulfinyl)pyrazino[1′,2′:1,5]pyrrolo[3,2-d]pyrimidin-4-yl)-6-methyl-1,4-oxazepan-6-ol. MS (ESI): m/z (M+H)⁺ 654.

### Step C: (6S)-4-(9-(16-chloro-5-cyclopropyl-1-(tetrahydro-2H-pyran-2-yl)-1H-indazol-4-yl)-10-fluoro-2-(((2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepam-6-ol

LiHMDS (0.32 mL, 0.32 mmol, 1 M in THF) was added to a solution of ((2R,7aS)-2-fluorotetrahydro-1H-pyrrolizin-7a(5H)-yl) methanol (40 mg, 0.25 mmol) in THF (1.5 mL) at 0° C. The reaction mixture was stirred at 0° C. for 30 min. (6S)-4-(9-(6-chloro-5-cyclopropyl-1-(tetrahydro-2H-pyran-2-yl)-1H-indazol-4-yl)-10-fluoro-2-(methylsulfinyl)pyrazino[1′,2′:1,5]pyrrolo[3,2-d]pyrimidin-4-yl)-6-methyl-1,4-oxazepan-6-ol (60 mg, 0.092 mmol) was added as a solution in THF (1.5 mL). After stirring at 0° C. for 30 min, the mixture was diluted with EtOAc, washed with sat. aq. NH₄Cl and brine, dried over Na₂SO₄, filtered, and evaporated. The crude residue obtained was purified by silica gel chromatography (CHCl₃/EtOH) to afford (6S)-4-(9-(6-chloro-5-cyclopropyl-1-(tetrahydro-2H-pyran-2-yl)-1H-indazol-4-yl)-10-fluoro-2-(((2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepan-6-ol. MS (ESI): m/z (M+H)⁺ 749.

### Step D: (S)-4-(9-(6-chloro-5-cyclopropyl-1H-indazol-4-yl)-10-fluoro-2-(((2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepan-6-ol (Ex-305)

TFA (0.60 mL) was added to a solution of (6S)-4-(9-(6-chloro-5-cyclopropyl-1-(tetrahydro-2H-pyran-2-yl)-1H-indazol-4-yl)-10-fluoro-2-(((2R,7aS)-2-fluorotetrahydro-H-pyrrolizin-7a(5H)-yl)methoxy)pyrazino[1′,2′:1,5]pyrrolo[3,2-d]pyrimidin-4-yl)-6-methyl-1,4-oxazepan-6-ol (55 mg, 0.073 mmol) in DCM (0.60 mL) at 0° C. After stirring at 0° C. for 3 h, the solution was diluted with CHCl₃ and quenched with sat. aq. NaHCO₃. The layers were separated and the aqueous phase was extracted with CHCl₃. The combined organic layers were washed with brine, dried over Na₂SO₄, filtered, and evaporated. The crude residue obtained was purified by preparative reverse-phase HPLC (MeCN/H₂O w/formic acid modifier), Sat. aq. NaHCO₃ was added to the purified fractions containing the title compound, followed by extraction with ethyl acetate. The extract was washed with brine and dried over Na₂SO₄. The solvent was evaporated under reduced pressure to afford(S)-4-(9-(6-chloro-5-cyclopropyl-1H-indazol-4-yl)-10-fluoro-2-(((2R,7aS)-2-fluorotetrahydro-1H-pyrrolizin-7a(5H)-yl)methoxy)pyrazino[1′,2′:1,5]pyrrolo[3,2-d]pyrimidin-4-yl)-6-methyl-1,4-oxazepan-6-ol (Ex-305). MS (ESI): m/z (M+H)⁺ 665. ¹H-NMR (400 MHz, CDCl₃) δ 7.95 (q, J=5.2 Hz, 1H), 7.89 (dd, J=13.0, 4.8 Hz, 1H), 7.82-7.69 (m, 1H), 7.67-7.64 (m, 1H), 5.35-5.21 (m, 1H), 4.31-4.27 (m, 1H), 4.22 (q, J=5.3 Hz, 1H), 4.03-3.98 (m, 2H), 3.87-3.71 (m, 3H), 3.66-3.60 (m, 1H), 3.45-3.34 (m, 2H), 3.28-3.17 (m, 3H), 2.98 (td, J=9.2, 3.7 Hz, 1H), 2.33-2.22 (m, 1H), 2.19-2.12 (m, 2H), 2.09-2.00 (m, 2H), 1.97-1.87 (m, 3H), 1.39-1.34 (m, 3H), 0.95-0.84 (m, 1H), 0.77-0.70 (m, 0.5H), 0.49-0.42 (m, 0.5H), 0.33-0.23 (m, 1H), 0.21-0.07 (m, 2H).

The example in the table below was synthesized using a similar procedure as described in the synthesis of Ex-305 by making the appropriate substitutions for starting material, intermediates, and/or reagents. Such starting materials, intermediates, and/or reagents are available commercially, synthesized as described in the literature, synthesized using methods available to those skilled in the art, or synthesized as described herein.

| | Starting | | | [M + H]⁺ |
|---|---|---|---|---|
| Ex. | Material | Structure | Compound Name | Found |
| Ex- 306 | 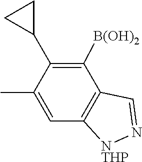 Int-28 | 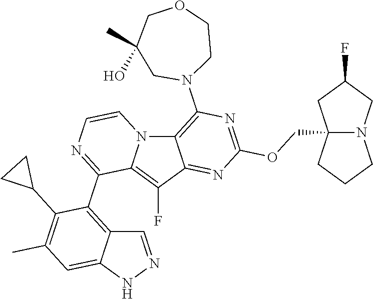 [SMILES: Cc1cc2[nH]ncc2c(-c2nccn3c2c(F)c2nc(OC[C@@]45CCCN4C[C@H](F)C5)nc(N4CCOC[C@@](C)(O)C4)c23)c1C1CC1] | (S)-4-(9-(5-cyclopropy1-6- methyl-1H-indazol-4-yl)-10- fluoro-2-(((2R,7aS)-2- fluorotetrahydro-1H- pyrrolizin-7a(5H)- yl)methoxy)pyrazino[1′2′:1,5] pyrrolo[3,2-d]pyrimidin-4-yl)- 6-methyl-1,4-oxazepan-6-o1 | 645 |

### Example 307: (6S)-4-(2′-(((2R,7aS)-2-fluorotetrahydro-1H-pyrrolizin-7a(5H)-yl)methoxy)-8′-(6-methyl-5-(trifluoromethyl)-1H-indazol-4-yl)spiro[cyclopropane-1,9′-pyrido[4′,3′:3,4]cyclopenta[1,2-d]pyrimidin]-4′-yl)-6-methyl-1,4-oxazepan-6-ol (Ex-307)

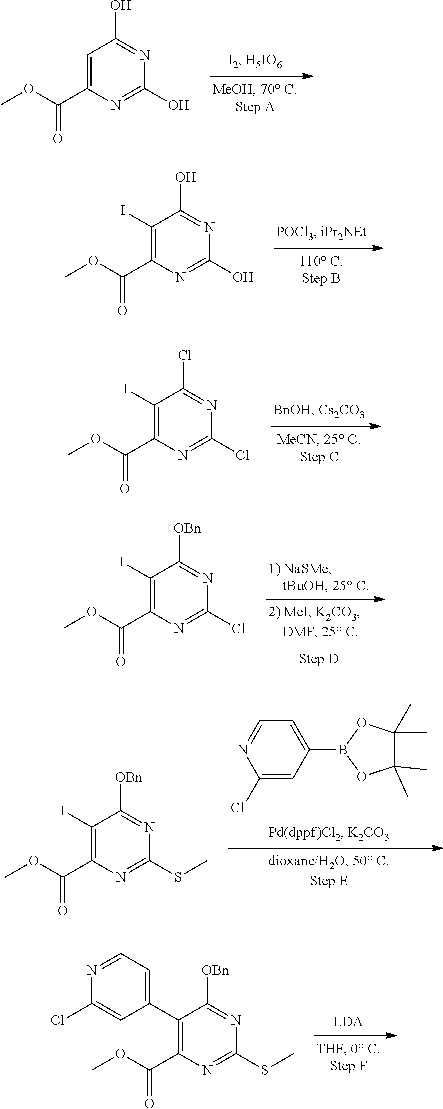

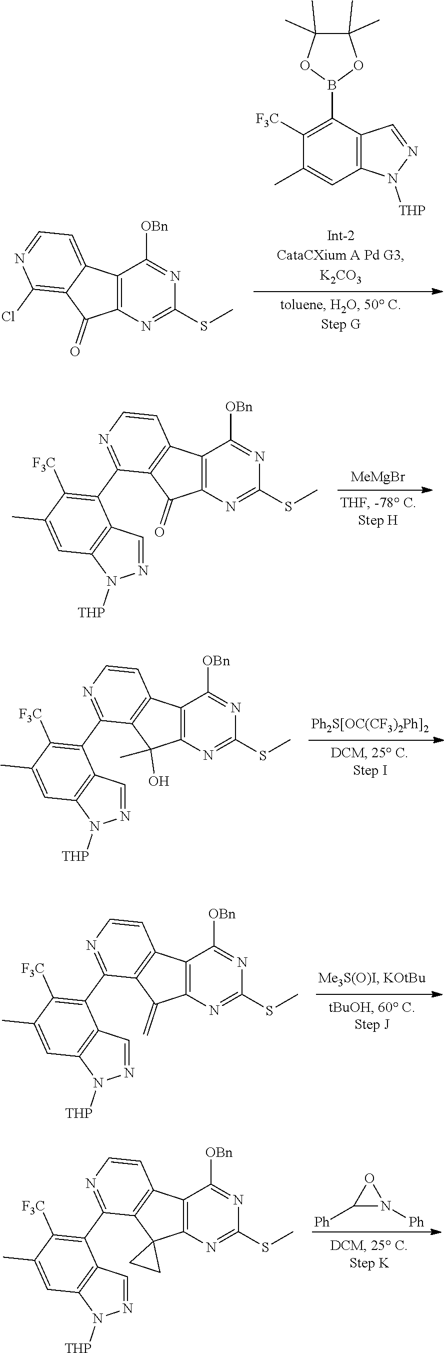

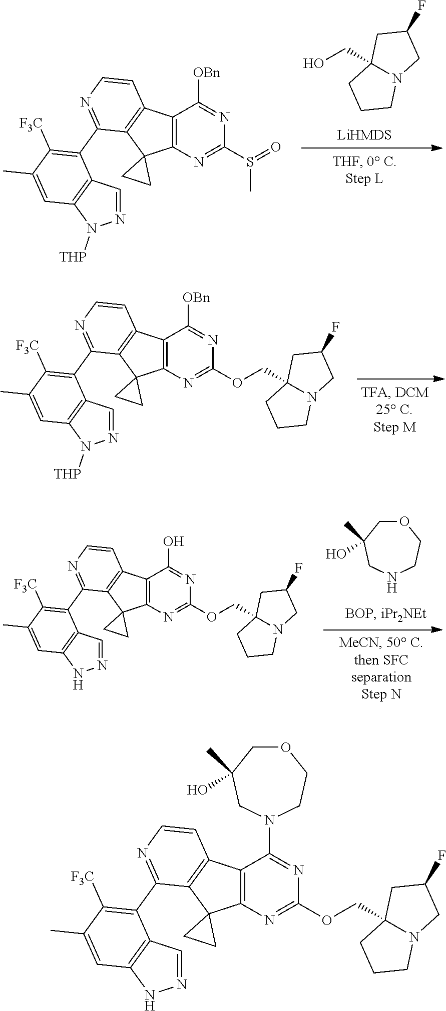

### Step A: Methyl 2,6-dihydroxy-5-iodopyrimidine-4-carboxylate

Periodic acid (9.04 g, 39.7 mmol) and iodine (23.50 g, 93 mmol) were added to a solution of methyl 2,6-dihydroxypyrimidine-4-carboxylate (37.5 g, 220 mmol) in MeOH (350 mL) at 25° C. The reaction mixture was heated to 70° C. and stirred for 16 h. The reaction mixture was concentrated in vacuo and the residue was diluted with H₂O (200 mL). The resulting mixture was filtered and the filter cake was washed with H₂O (2×50 mL).

The filter cake was dried via azeotropic distillation using toluene to give methyl 2,6-dihydroxy-5-iodopyrimidine-4-carboxylate. MS (ESI) [M+H]⁺: m/z 297.

### Step B: Methyl 2,6-dichloro-5-iodopyrimidine-4-carboxylate

N,N-diisopropylethylamine (73.0 mL, 418 mmol) was added to a solution of methyl 2,6-dihydroxy-5-iodopyrimidine-4-carboxylate (55 g, 186 mmol) in POCl₃ (550 mL) at 25° C. The reaction mixture was heated to 110° C. for 6 h. POCl₃ was removed by distillation followed by azeotropic distillation using toluene (2×200 mL). The crude residue was purified by flash silica gel chromatography (petroleum ether/EtOAc=10/1) to give methyl 2,6-dichloro-5-iodopyrimidine-4-carboxylate. ¹H NMR (400 MHz, CDCl₃) δ 4.05 (s, 3H).

### Step C: Methyl 6-(benzyloxy)-2-chloro-5-iodopyrimidine-4-carboxylate

Cs₂CO₃ (63.7 g, 196 mmol) was added to a solution of methyl 2,6-dichloro-5-iodopyrimidine-4-carboxylate (37.2 g, 112 mmol) and benzyl alcohol (10.88 g, 101 mmol) in MeCN (375 mL) at 25° C. The reaction mixture was stirred at 25° C. for 12 h. The reaction mixture was diluted with EtOAc (375 mL), filtered, and the filtrate was concentrated in vacuo. The residue was purified by flash silica gel chromatography (petroleum ether/EtOAc=20/1) to give methyl 6-(benzyloxy)-2-chloro-5-iodopyrimidine-4-carboxylate. MS (ESI) [M+H]⁺: m/z 405.

### Step D: Methyl 6-(benzyloxy)-5-iodo-2-(methylthio)pyrimidine-4-carboxylate

Sodium methanethiolate (8.45 g, 121 mmol) was added to a solution of methyl 6-(benzyloxy)-2-chloro-5-iodopyrimidine-4-carboxylate (28.7 g, 70.9 mmol) in tert-butanol (450 mL) at 25° C. and the reaction mixture was stirred for 16 h under N₂ atmosphere. The reaction mixture was quenched with H₂O (100 mL) and extracted with EtOAc (3×500 mL). The organic layer was dried over Na₂SO₄, filtered, and concentrated in vacuo to give 6-(benzyloxy)-5-iodo-2-(methylthio)pyrimidine-4-carboxylic acid, which was used to next step without further purification. MS (ESI) [M+H]⁺: m/z 403.

K₂CO₃ (49.0 g, 354 mmol) and MeI (17.72 mL, 283 mmol) was added to a solution of 6-(benzyloxy)-5-iodo-2-(methylthio)pyrimidine-4-carboxylic acid (28.5 g, 70.9 mmol) in DMF (300 mL) at 25° C. and the reaction mixture was stirred for 16 h under N₂ atmosphere. The reaction mixture was diluted with H₂O (100 mL) and extracted with EtOAc (3×300 mL). The combined organic layer was washed with brine (3×40 mL), dried over Na₂SO₄, filtered, and concentrated in vacuo. The residue was purified by flash silica gel chromatography (0 to 18% petroleum Ether in THF) to afford methyl 6-(benzyloxy)-5-iodo-2-(methylthio)pyrimidine-4-carboxylate. MS (ESI) [M+H]⁺: m/z 417.

### Step E: 6-(benzyloxy)-5-(2-chloropyridin-4-yl)-2-(methylthio)pyrimidine-4-carboxylate

2-chloro-4-(4,4,5,5-tetramethyl-1,3,2-dioxaborolan-2-yl)pyridine (6.28 g, 26.2 mmol), K₂CO₃ (7.77 g, 56.2 mmol) and Pd(dppf)Cl₂ (2.74 g, 3.75 mmol) were added to a solution of methyl 6-(benzyloxy)-5-iodo-2-(methylthio)pyrimidine-4-carboxylate (7.8 g, 18.74 mmol) in dioxane (100 mL) under N₂ atmosphere and the reaction mixture was stirred at 50° C. for 4 h. The reaction mixture was diluted with H₂O (40 mL) and the resulting mixture was extracted with EtOAc (3×200 mL). The combined organic phase was washed with brine, dried over anhydrous Na₂SO₄, filtered, and concentrated in vacuo. The residue was purified by flash silica gel chromatography (petroleum ether/EtOAc=5/1) to give methyl 6-(benzyloxy)-5-(2-chloropyridin-4-yl)-2-(methylthio)pyrimidine-4-carboxylate. MS (ESI) [M+H]⁺: m/z 402.

### Step F: 4-(benzyloxy)-8-chloro-2-(methylthio)-9H-pyrido[4′,3′:3,4]cyclopenta[1,2-d]pyrimidin-9-one

The reaction was set up using flow chemistry. Methyl 6-(benzyloxy)-5-(2-chloropyridin-4-yl)-2-(methylthio)pyrimidine-4-carboxylate (5.6 g, 13.94 mmol) was dissolved in THF (120 mL) and charged into Reservoir 1 at 20° C. LDA (20.90 mL, 41.8 mmol, 2 M in THF) and THF (120 mL) was charged into Reservoir 2 at 20° C. The flow rate for Reservoir 1 and Reservoir 2 was adjusted to 12.0 mL/min with the total reaction residence time being 15 s. The reaction mixture was quenched with aq. NH₄Cl, the organic phase was collected, and the pumps and tubing were washed with THF. The reaction mixture was diluted with additional sat. aq. NH₄Cl (80 mL) and the mixture was extracted with EtOAc (200 mL×3). The combined organic layers were washed with brine, dried over anhydrous Na₂SO₄, filtered, and concentrated in vacuo. The residue was purified by flash silica gel chromatography (petroleum ether/THF=5/1) to give 4-(benzyloxy)-8-chloro-2-(methylthio)-9H-pyrido[4′,3′:3,4]cyclopenta[1,2-d]pyrimidin-9-one. MS (ESI) [M+H]⁺: m/z 370.

### Step G: 4-(benzyloxy)-8-(6-methyl-1-(tetrahydro-2H-pyran-2-yl)-5-(trifluoromethyl)-1H-indazol-4-yl)-2-(methylthio)-9H-pyrido[4′,3′:3,4]cyclopenta[1,2-d]pyrimidin-9-one

Potassium carbonate (10.55 mL, 21.09 mmol, 2 M in H₂O), cataCXium A Pd G3 (2.350 g, 3.52 mmol), and 6-methyl-1-(tetrahydro-2H-pyran-2-yl)-4-(4,4,5,5-tetramethyl-1,3,2-dioxaborolan-2-yl)-5-(trifluoromethyl)-1H-indazole (Int-2) (3.17 g, 7.73 mmol) was added to a solution of 4-(benzyloxy)-8-chloro-2-(methylthio)-9H-pyrido[4′,3′:3,4]cyclopenta[1,2-d]pyrimidin-9-one (2.6 g, 7.03 mmol) in toluene (60 mL) at 25° C. The reaction mixture was heated to 50° C. for 16 h under N₂ atmosphere. The reaction mixture was cooled to room temperature, diluted with EtOAc (150 mL), and filtered. The filtrate was washed with brine (3×20 mL), dried over sodium sulfate, filtered, and the filtrate was concentrated in vacuo. The residue was purified by flash silica gel chromatography (EtOAc/petroleum ether=1:3) to afford 4-(benzyloxy)-8-(6-methyl-1-(tetrahydro-2H-pyran-2-yl)-5-(trifluoromethyl)-1H-indazol-4-yl)-2-(methylthio)-9H-pyrido[4′,3′:3,4]cyclopenta[1,2-d]pyrimidin-9-one. MS (ESI) [M+H]⁺: m/z 618.

### Step H: 4-(benzyloxy)-9-methyl-8-(6-methyl-1-(tetrahydro-2H-pyran-2-yl)-5-(trifluoromethyl)-1H-indazol-4-yl)-2-(methylthio)-9H-pyrido[4′,3′:3,4]cyclopenta[1,2-d]pyrimidin-9-ol

Methylmagnesium bromide (2.159 mL, 6.48 mmol, 3 M in THF) was added to a solution of 4-(benzyloxy)-8-(6-methyl-1-(tetrahydro-2H-pyran-2-yl)-5-(trifluoromethyl)-1H-indazol-4-yl)-2-(methylthio)-9H-pyrido[4′,3′:3,4]cyclopenta[1,2-d]pyrimidin-9-one (2 g, 3.24 mmol) in THF (20 mL) at −78° C., and the reaction mixture was stirred for 40 min under N₂ atmosphere. The reaction mixture was quenched with sat. aq. NH₄Cl (5 mL) and extracted with EtOAc (3×50 mL). The combined organic layers were washed with brine (3×5 mL), dried over sodium sulflate, filtered, and the filtrate was concentrated in vacuo to give a residue. The residue was purified by flash silica gel chromatography (petroleum ether:EtOAc=3:1) to afford 4-(benzyloxy)-9-methyl-8-(6-methyl-1-(tetrahydro-2H-pyran-2-yl)-5-(trifluoromethyl)-1H-indazol-4-yl)-2-(methylthio)-9H-pyrido[4′,3′:3,4]cyclopenta[1,2-d]pyrimidin-9-ol. MS (ESI) [M+H]⁺: m/z 634.

### Step I: 4-(benzyloxy)-8-(6-methyl-1-(tetrahydro-2H-pyran-2-yl)-5-(trifluoromethyl)-1H-indazol-4-yl)-9-methylene-2-(methylthio)-9H-pyrido[4′,3′:3,4]cyclopenta[1,2-d]pyrimidine

In a glovebox, Martin's sulfurane (1.910 g, 2.84 mmol) was added to a solution of 4-(benzyloxy)-9-methyl-8-(6-methyl-1-(tetrahydro-2H-pyran-2-yl)-5-(trifluoromethyl)-1H-indazol-4-yl)-2-(methylthio)-9H-pyrido[4′,3′:3,4]cyclopenta[1,2-d]pyrimidin-9-ol (1.2 g, 1.894 mmol) in DCM (20 mL) at 25° C. and the reaction mixture was stirred at for 1 h. The reaction mixture was concentrated in vacuo and the residue was purified by flash silica gel chromatography (petroleum ether/THF=3/1) to give 4-(benzyloxy)-8-(6-methyl-1-(tetrahydro-2H-pyran-2-yl)-5-(trifluoromethyl)-1H-indazol-4-yl)-9-methylene-2-(methylthio)-9H-pyrido[4′,3′:3,4]cyclopenta[1,2-d]pyrimidine. MS (ESI) [M+H]⁺: m/z 616.

### Step 4′ (benzyloxy)-8′-(6-methyl-1-(tetrahydro-2H-pyran-2-yl)-5-(trifluoromethyl)-1H-indazol-4-yl)-2′-(methylthio)spiro[cyclopropane-1,9′-pyrido[4′,3′:3,4]cyclopenta[1,2-d]pyrimidine]

Potassium tert-butoxide (142 mg, 1.267 mmol) was added to a solution of trimethylsulfoxonium iodide (279 mg, 1.267 mmol) in t-BuOH (4 mL) at 25° C. under N₂ atmosphere and the mixture was stirred at for 1 h. 4-(benzyloxy)-8-(6-methyl-1-(tetrahydro-2H-pyran-2-yl)-5-(trifluoromethyl)-1H-indazol-4-yl)-9-methylene-2-(methylthio)-9H-pyrido[4′,3′:3,4]cyclopenta[1,2-d]pyrimidine (600 mg, 0.975 mmol) in t-BuOH (8 mL) was added to the above mixture and the reaction mixture was stirred at 60° C. for 1 h. The mixture was cooled to room temperature and quenched with sat. aq. NH₄Cl (5 mL). The reaction mixture was extracted with EtOAc (3×20 mL). The combined organic layers were washed with brine (2×10 mL), dried over sodium sulfate, filtered, and the filtrate was concentrated in vacuo. The residue was purified by flash silica gel chromatography (petroleum ether/EtOAc=5/1) to give 4′-(benzyloxy)-8′-(6-methyl-1-(tetrahydro-2H-pyran-2-yl)-5-(trifluoromethyl)-1H-indazol-4-yl)-2′-(methylthio)spiro[cyclopropane-1,9′-pyrido[4′,3′:3,4]cyclopenta[1,2-d]pyrimidine]. MS (ESI) [M+H]⁺: m/z 630.

### Step K: 4′-(benzyloxy)-8′-(6-methyl-1-(tetrahydro-2H-pyran-2-yl)-5-(trifluoromethyl)-1H-indazol-4-yl)-2′-(methylsulfinyl)spiro[cyclopropane-1,9′-pyrido[4′,3′:3,4]cyclopenta[1,2-d]pyrimidine]

3-phenyl-2-(phenylsulfonyl)-1,2-oxaziridine (448 mg, 1.715 mmol) was added to a solution of 4′-(benzyloxy)-8′-(6-methyl-1-(tetrahydro-2H-pyran-2-yl)-5-(trifluoromethyl)-1H-indazol-4-yl)-2′-(methylthio)spiro[cyclopropane-1,9′-pyrido[4′,3′:3,4]cyclopenta[1,2-d]pyrimidine](360 mg, 0.572 mmol) in DCM (5 mL) at 25° C. under N₂ atmosphere and the mixture was stirred for 1 h. The reaction mixture was concentrated in vacuo at 35° C. and the residue was purified by silica gel chromatography (petroleum ether/THF=1/1) to give 4′-(benzyloxy)-8′-(6-methyl-1-(tetrahydro-2H-pyran-2-yl)-5-(trifluoromethyl)-1H-indazol-4-yl)-2′-(methylsulfinyl)spiro[cyclopropane-1,9′-pyrido[4′,3′:3,4]cyclopenta[1,2-d]pyrimidine]. MS (ESI) [M+H]⁺: m/z 646.

### Step L: 4′-(benzyloxy)-2′-(((2R,7aS)-2-fluorotetrahydro-1H-pyrrolizin-7a(5H)-yl)methoxy)-8′-(6-methyl-1-(tetrahydro-2H-pyran-2-yl)-5-(trifluoromethyl)-1H-indazol-4-yl)spiro[cyclopropane-1,9′ pyrido[4′,3′:3.4]cyclopenta[1,2-d]pyrimidine]

4′-(benzyloxy)-8′-(6-methyl-1-(tetrahydro-2H-pyran-2-yl)-5-(trifluoromethyl)-1H-indazol-4-yl)-2′-(methylsulfinyl)spiro[cyclopropane-1,9′-pyrido[4′,3′:3,4]cyclopenta[1,2-d]pyrimidine](280 mg, 0.434 mmol) was added to a solution of ((2R,7aS)-2-fluorotetrahydro-1H-pyrrolizin-7a(5H)-yl) methanol (117 mg, 0.737 mmol) in THF (3 mL) at 25° C. LiHMDS (0.667 mL, 0.867 mmol, 1.3 M in THF) was added to the reaction mixture at 0° C. and the reaction mixture was stirred for 0.5 h. The reaction mixture was quenched with sat. aq. NH₄Cl (2 mL) and the resulting mixture was extracted with EtOAc (3×20 mL), The combined organic layers were washed with brine, dried over anhydrous Na₂SO₄, filtered, and concentrated in vacuo. The residue was purified by preparative TLC (SiO₂, Pet. ether/THF=1/1) to give 4′-(benzyloxy)-2′-(((2R,7aS)-2-fluorotetrahydro-1H-pyrrolizin-7a(5H)-yl)methoxy)-8′-(6-methyl-1-(tetrahydro-2H-pyran-2-yl)-5-(trifluoromethyl)-1H-indazol-4-yl)spiro[cyclopropane-1,9′-pyrido[4′,3′:3,4]cyclopenta[1,2-d]pyrimidine]. MS (ESI) [M+H]⁺: m/z 741.

### Step M: 2′-(((2R,7aS)-2-fluorotetrahydro-1H-pyrrolizin-7a(5H)-yl)methoxy)-8′-(6-methyl-5-(trifluoromethyl)-1H-indazol-4-yl)spiro[cyclopropane-1,9′-pyrido[4′,3′:3,4]cyclopenta[1,2-d]pyrimidin]-4′-ol

TFA (1.5 mL, 19.47 mmol) was added to a solution of 4′-(benzyloxy)-2′-(((2R,7aS)-2-fluorotetrahydro-1H-pyrrolizin-7a(5H)-yl)methoxy)-8′-(6-methyl-1-(tetrahydro-2H-pyran-2-yl)-5-(trifluoromethyl)-1H-indazol-4-yl)spiro[cyclopropane-1,9′-pyrido[4′,3′:3,4]cyclopenta[1,2-d]pyrimidine](260 mg, 0.351 mmol) in DCM (4.5 mL) at 25° C. and the reaction mixture was stirred for 1 h. The reaction mixture was concentrated in vacuo and the residue was diluted with DCM (30 mL, contained 10% MeOH). The resulting mixture was basified with sat. aq. NaHCO₃ (3 mL) and extracted with DCM (2×20 mL). The combined organic phase was washed with brine, dried over anhydrous Na₂SO₄, filtered, and concentrated in vacuo to give 2′-(((2R,7aS)-2-fluorotetrahydro-1H-pyrrolizin-7a(5H)-yl)methoxy)-8′-(6-methyl-5-(trifluoromethyl)-1H-indazol-4-yl)spiro[cyclopropane-1,9′-pyrido[4′,3′:3,4]cyclopenta[1,2-d]pyrimidin]-4′-ol. MS (ESI) [M+H]⁺: m/z 567.

### Step N: (6.5)-4-(2′-(((2R,7aS)-2-fluorotetrahydro-1H-pyrrolizin-7a(5H)-yl)methoxy)-8′-(6-methyl-5-(trifluoromethyl)-1H-indazol-4-yl)spiro[cyclopropane-1,9′-pyrido[4′,3′:3,4]cyclopenta[1,2-d]pyrimidin]-4′-yl)-6-methyl-1,4-oxazepan-6-ol (Ex-307)

BOP (211 mg, 0.477 mmol) was added to a solution of 2′-(((2R,7aS)-2-fluorotetrahydro-1H-pyrrolizin-7a(5H)-yl)methoxy)-8′-(6-methyl-5-(trifluoromethyl)-1H-indazol-4-yl)spiro[cyclopropane-1,9′-pyrido[4′,3′:3,4]cyclopenta[1,2-d]pyrimidin]-4′-ol (180 mg, 0.318 mmol) in MeCN (5 mL) at 25° C., and the mixture was stirred at 25° C. for 1 h. Then(S)-6-methyl-1,4-oxazepan-6-ol hydrochloride (160 mg, 0.953 mmol) and N,N-diisopropylethylamine (0.277 mL, 1.589 mmol) were added the reaction mixture and the mixture was stirred at 50° C. for 16 h. The reaction mixture was cooled to room temperature, filtered, and the filtrate was purified by preparative HPLC (MeCN/H₂O w/TFA modifier) to give racemic (6S)-4-(2′-(((2R,7aS)-2-fluorotetrahydro-1H-pyrrolizin-7a(5H)-yl)methoxy)-8′-(6-methyl-5-(trifluoromethyl)-1H-indazol-4-yl)spiro[cyclopropane-1,9′-pyrido[4′,3′:3,4]cyclopenta[1,2-d]pyrimidin]-4′-yl)-6-methyl-1,4-oxazepan-6-ol. MS (ESI) [M+H]⁺; m/z 680. The atropisomers of (6S)-4-(2′-(((2R,7aS)-2-fluorotetrahydro-1H-pyrrolizin-7a(5H)-yl)methoxy)-8′-(6-methyl-5-(trifluoromethyl)-1H-indazol-4-yl)spiro[cyclopropane-1,9′-pyrido[4′,3′:3,4]cyclopenta[1,2-d]pyrimidin]-4′-yl)-6-methyl-1,4-oxazepan-6-ol (80 mg, 0.118 mmol) were separated by preparative SFC (Column B, 30 to 100% EtOH in CO₂ w/0.1% NH₄OH modifier) to provide (6S)-4-(2′-(((2R,7aS)-2-fluorotetrahydro-1H-pyrrolizin-7a(5H)-yl)methoxy)-8′-(6-methyl-5-(trifluoromethyl)-1H-indazol-4-yl)spiro[cyclopropane-1,9′-pyrido[4′,3′:3,4]cyclopenta[1,2-d]pyrimidin]-4′-yl)-6-methyl-1,4-oxazepan-6-ol (Ex-307, the second eluting isomer from SFC), ¹H NMR (400 MHz, CD₃OD) δ 8.58 (d, J=5.4 Hz, 1H), 7.80-7.69 (m, 2H), 7.43 (s, 1H), 5.40-5.18 (m, 1H), 4.27-4.16 (m, 2H), 4.15-4.00 (m, 3H), 3.97-3.82 (m, 2H), 3.79-3.70 (m, 2H), 3.47 (d, J=12.5 Hz, 1H), 3.23 (br s, 2H), 3.18-3.12 (m, 1H), 3.07-2.95 (m, 1H), 2.73 (s, 3H), 2.33-2.15 (m, 2H), 2.10-2.05 (m, 1H), 2.03-1.92 (m, 2H), 1.92-1.81 (m, 1H), 1.79-1.70 (m, 1H), 1.59-1.51 (m, 1H), 1.49-1.40 (m, 1H), 1.27 (s, 3H), 0.88-0.78 (m, 1H). MS (ESI) [M+H]⁺: m/z 680.

### Assays

### Procedure for SOS-Catalyzed Nucleotide Exchange Assay for KRAS-WT, G12C/D/V, G13D, HRAS, and NRAS (Procedure A)

Recombinant KRAS-G12C protein used in this assay has an additional triple mutation (C51S/C80L/C118S). Whereas KRAS-WT, G12D/V, G13D, HRAS and NRAS are in context of their WT protein sequence background. Specifically, the SOS-catalyzed nucleotide exchange assay utilizes a preformed TR-FRET complex containing a specific biotinylated RAS protein (KRAS-WT, G12C/V/D, G13D, HRAS, NRAS; described above) with Bodipy-GDP, and Terbium-streptavidin. Compounds are preincubated with this complex for 60 minutes. Subsequently, recombinant human SOS protein and unlabeled GTP are added to initiate the exchange reaction. Small molecule inhibitors stabilize the Bodipy-GDP complex whereas the untreated protein rapidly exchanges Bodipy-GDP for unlabeled GTP resulting in reduced TR-FRET signal.

To assemble the preformed TR-FRET complexes, each biotinylated RAS protein is diluted to 2 μM in an EDTA Buffer (20 mM HEPES pH 7.5, 50 mM sodium chloride, 10 mM EDTA, and 0.01% Tween) and incubated at room temperature for one hour. This mixture is then further diluted to 90 nM in an Assay Buffer (20 mM HEPES pH 7.5, 150 mM sodium chloride, 10 mM magnesium chloride, and 0.005% Tween) containing 15 nM of Terbium-Streptavidin (Invitrogen, catalog #PV3577) and 900 nM of Bodipy-GDP (Invitrogen, catalog #G22360) and incubated at room temperature for six hours. It should be noted that this preformed TR-FRET complex for each of the RAS protein were made ahead of time, aliquoted and stored at −80° C. until the day of the experiment.

Each test compound (10 mM stock in DMSO) is diluted in DMSO to make a final-10-point, 3-fold dilution and is acoustically dispensed into a 384-well assay plate (Corning, catalog #3820) using an Echo 550 (Labcyte). Each well of the assay plate receives 3 μL of a specific 3×RAS preformed TR-FRET complex and 3 μL of Assay Buffer and is incubated at room temperature for 60 minutes (preincubation time). Each well then receives 3 μL of 3×recombinant human SOS protein and GTP (Sigma, G8877) in Assay Buffer and is incubated at room temperature for 30 minutes (G13D), 60 minutes (KRAS WT/G12C/D, H/NRAS) or 90 minutes for G12V. The final reaction in each well of 9 μL consists of 3 mM GTP, specific Ras and SOS proteins in the following concentrations: KRAS-G12C/SOS=3 nM/40 nM, KRAS-WT and G12D/SOS=1 nM/40 nM, KRAS-G12V/SOS=1.25 nM/160 nM. KRAS-G13D/SOS=1.25 nM/0 nM, HRAS and NRAS=1.25 nM/40 nM.

The time-resolved fluorescence resonance energy transfer (TR-FRET) signal is measured on an Envision (PerkinElmer) plate reader: Excitation filter=340 nm; emission 1=495 nm; emission2=520 nm; dichroic mirror=D400/D505; delay time=100 ms. The signal of each well is determined as the ratio of the emission at 520 nm to that at 495 nm. Percent effect of each well is determined after normalization to control wells containing DMSO (no effect) or a saturating concentration of inhibitor (max effect). The apparent effect as a function of compound concentration is fit to a four-parameter logistic equation.

### Procedure for SOS-Catalyzed Nucleotide Exchange Assay for KRAS-G12C/D/V/WT (Procedure B)

Recombinant KRAS G12C (amino acids 1-169, SEQ ID NO:9), KRAS G12D (amino acids 1-169, SEQ ID NO: 10), KRAS G12V (amino acids 1-169, SEQ ID NO: 11), KRAS WT (amino acids 1-169, SEQ ID NO:12) and SOS1 (amino acids 564-1049, SEQ ID NO: 13) proteins were expressed in E. coli and purified by affinity chromatography. To prepare each BODIPY™ FL GDP-bound KRAS protein, 50 μM KRAS proteins were incubated with 0.5 mM BODIPY™ FL GDP (Invitrogen, G22360) in a loading buffer (20 mM Tris-HCl pH 7.5, 50 mM NaCl, 1 mM DTT and 2.5 mM EDTA) for 1 hour on ice. After the incubation, MgCl₂ was added to a final concentration of 10 mM, followed by an incubation at room temperature for 30 minutes. The mixtures were allowed to pass through a NAP-5 column to remove free nucleotides and purified BODIPY™ FL GDP-bound KRAS G12C, G12D, G12V and WT proteins were used for compound evaluation.

The inhibitory activity of compounds on recombinant KRAS is measured by the displacement of the bound BODIPY™ GDP. Specifically, 2.5 nM of each BODIPY™ FL GDP-bound KRAS complex was incubated with various concentrations of compound in a reaction buffer (20 mM Tris-HCl pH 7.5, 100 mM NaCl, 1 mM MgCl₂, 2 mM DTT, 0.1% Tween 20) at 25° C. for 1 hour. After the incubation, recombinant SOS1 and GMPPNP (Jena Bioscience GmbH, NU-401) were added and incubated at room temperature for 30 minutes to proceed SOS1-dependent GDP-GTP exchange reaction on KRAS. Displacement of BODIPY™ FL GDP by Guanosine-5′-[(β,γ)-imido]triphosphate, Tetralithium salt (GMPPNP) was measured by calculating the ratio of fluorescence intensities of BODIPY™ FL before and after the exchange reaction. Percent Inhibition was calculated by setting the fluorescence ratio from the reaction without test compound (DMSO control) and the fluorescence ratio from the reaction without SOS1 and GMPPNP as 0% and 100% inhibition, respectively. Dose response curves were analyzed using a 4-parameter logistic model to calculate IC₅₀ values.

### Procedure for Cellular Phospho-ERK Assay in KRAS Wild Type and Mutant (G12D, G12V) Cell Lines

MKN-1 cells (JCRB JCRB0252) containing amplified wild-type KRAS, were cultured in growth medium that contains RPMI 1640-GlutaMAX™-I (ThermoFisher Scientific 61870) containing 10% heat inactivated fetal bovine serum (ThermoFisher Scientific 10091148), 1 mM sodium pyruvate and 10 mM HEPES. PANC08.13 cells (ATCC® CRL-2551™), containing homozygous KRAS-G12D activating mutation, were cultured in growth medium made of RPMI1640-GlutaMAX™-I (ThermoFisher Scientific 61870) containing 15% heat inactivated fetal bovine serum (ThermoFisher Scientific 10091148)). AsPC-1 cells (ATCC® CRL-1682™), containing homozygous KRAS-G12D activating mutation, were cultured in T150 flask in growth medium (RPMI medium 1640-GlutaMAX™-I (ThermoFisher Scientific 61870) containing 10% fetal bovine serum (ThermoFisher Scientific 10091148)). SW620 cells (ATCC® CRL-227™), containing homozygous KRAS-G12V activating mutation, were cultured in growth medium that contains RPMI 1640-GlutaMAX™-I (ThermoFisher Scientific 61870) containing 10% heat inactivated fetal bovine serum (ThermoFisher Scientific 10091148).

Cells for the assay were harvested in growth medium after TrypLE (ThermoFisher scientific 12604021) digestion and were seeded in a 384-well collagen coated cell culture plate (Corning 356702) at a density of 10,000-15,000 cells/20 μL/well, and incubated at 37° C., 5% CO₂ overnight. The compound (with 10 mM stock concentration) dose-response titrations were prepared [30 μM final ERK detection assay concentration and 1:3 dilutions, 10-point dose response] and appropriate amounts (270 nL) of test compounds were dispensed in a 384-well intermediate plate using an Echo 550 liquid handler. 30 μL/well of RPMI medium 1640-GlutaMAX™—I was added to the intermediate plate and the contents of the intermediate plate (10 μL/well) were then transferred to the 384-well collagen coated cell culture plate, which was incubated at 37° C., 5% CO₂ for 2 hours. After removal of medium from the collagen coated cell culture plate, cells were lysed in lysis buffer from Alpha SureFire® Ultra™ Multiplex p-ERK and total ERK assay kit (PerkinElmer MPSU-PTERK) containing Halt™ Protease and Phosphatase inhibitor cocktail (ThermoFisher Scientific 78446) at room temperature with constant shaking at 300 rpm for 30 minutes. The cell lysates were then transferred to an OptiPlate-384 plate (PerkinElmer 6005620), and the phosphorylation of ERK (p-ERK) and total ERK levels were detected by Alpha SureFire® Ultra™ Multiplex p-EEK kit and total ERK assay kit (PerkinElmer MPSU-PTERK) following the manufacturer's protocol. Assay plates were read on a En Vision Multimode Plate Reader (PerkinElmer), and the ratio of p-ERK vs. total ERK in each well was used as the final readout. Dose response curves were analyzed using a 4-parameter logistic model to calculate IC₅₀ values using Spotfire software. The results of this assay are presented in the table below.

| | KRAS | KRAS | KRAS | KRAS | KRAS | | | PANC- | | | |
|---|---|---|---|---|---|---|---|---|---|---|---|
| | G12D | G12V | WT | G12C | G13D | HRAS | NRAS | 08.13 | AsPC1 | SW620 | MKN1 |
| | GNE | GNE | GNE | GNE | GNE | GNE | GNE | pERK | pERK | pERK | pERK |
| | IC50 | IC50 | IC50 | IC50 | IC50 | IC50 | IC50 | IC50 | IC50 | IC50 | IC50 |
| Ex # | (nM) | (nM) | (nM) | (nM) | (nM) | (nM) | (nM) | (nM) | (nM) | (nM) | (nM) |
| Ex-1 | 41.48 | 55.57 | 93.36 | 19.79 | 77.42 | 11060 | 11060 | 237.4 | 375.7 | 428.9 | 222.5 |
| Ex-2 | 35.41 | 36.77 | 64 | — | — | — | — | — | 425.7 | — | — |
| Ex-3 | 10.47 | 15.33 | 17.22 | — | — | — | — | — | 207 | 95.5 | 134.4 |
| Ex-4 | 338.6 | 536.2 | 567.9 | 123.9 | 429.9 | 11060 | 11060 | 1635 | — | — | — |
| Ex-5 | 61.24 | 80.65 | 100.9 | 96.82 | 94.32 | 11060 | 11060 | 188.5 | 693.7 | — | 127.3 |
| Ex-6 | 98.08 | 127.7 | 218.5 | 104.3 | 48.98 | 11060 | 11060 | 188.1 | 459.9 | 290.6 | 303.7 |
| Ex-7 | 22.29 | 30.22 | 42.85 | 129.4 | 22.88 | 11060 | 11060 | 99.87 | 248.1 | 232.7 | 141.8 |
| Ex-8 | 1497 | 1653 | 2246 | 1192 | 2090 | 11060 | 11060 | 1431 | — | — | — |
| Ex-9 | 616 | 575.4 | 1352 | 566.1 | 560.1 | 11060 | 11060 | 1688 | — | — | — |
| Ex-10 | 40.33 | 54.03 | 97.7 | 30.05 | 93.85 | 11060 | 11060 | 191.3 | 439.1 | 336.8 | 199.5 |
| Ex-11 | 84.17 | 124.9 | 158 | 41.09 | 104.7 | 11060 | 11060 | 239.6 | 1200 | — | — |
| Ex-12 | 2153 | 2965 | 5932 | 583.8 | 2654 | 11060 | 11060 | 6055 | — | — | — |
| Ex-13 | 3656 | 1662 | 2751 | 1485 | 4858 | 11060 | 11060 | 4303 | — | — | — |
| Ex-14 | 275.8 | 403.8 | 680.8 | 232.8 | 543 | 11060 | 11060 | 561.3 | — | — | — |
| Ex-15 | 9045 | 4928 | 8021 | 1971 | 5378 | 11060 | 11060 | — | — | — | — |
| Ex-16 | 952 | 561.2 | 765 | 1319 | 315.3 | 11060 | 11060 | 5449 | — | 8488 | 1773 |
| Ex-17 | 1418 | 1015 | 1808 | 300.1 | 591.6 | 11060 | 11060 | 3752 | — | — | — |
| Ex-18 | 1.148 | 3.846 | 1.459 | 10.47 | 2.659 | 1817 | 8817 | 4.54 | 29.76 | 13.12 | 7.67 |
| Ex-19 | 26.5 | 67.24 | 85.48 | 37.38 | 68.34 | 11060 | 11060 | 68.21 | 58.46 | 214 | 118 |
| Ex-20 | 37.93 | 62.86 | 125.3 | — | — | — | — | 80.62 | — | 538.9 | 150.3 |
| Ex-21 | 58.99 | 34.37 | 454.7 | 25.29 | 11060 | 11060 | 11060 | 1861 | 3261 | — | — |
| Ex-22 | 2445 | 409.3 | 974.9 | 189 | 456 | 11060 | 11060 | 3513 | — | — | — |
| Ex-23 | 1431 | 656.9 | 1205 | 189.8 | 788.5 | 11060 | 11060 | 3055 | — | — | — |
| Ex-24 | 704.6 | 491.6 | 929.4 | 224.2 | 1015 | 11060 | 11060 | 748.6 | — | — | — |
| Ex-25 | 61.61 | 89.84 | 135.5 | — | — | — | — | — | 1148 | — | — |
| Ex-26 | 95.97 | 96.33 | 189.5 | 208.7 | 94.3 | 11060 | 11060 | 169.5 | | 708.6 | 325.2 |
| Ex-27 | 196.5 | 221.1 | 792.9 | — | — | — | — | — | 660 | — | — |
| Ex-28 | 628.9 | 934.1 | 973.1 | 274.7 | 540.8 | 11060 | 11060 | 2043 | | — | — |
| Ex-29 | 250.9 | 142.7 | 851.8 | — | — | — | — | — | 1532 | — | — |
| Ex-30 | 157.9 | 196.5 | 436 | — | — | — | — | — | 730.2 | — | — |
| Ex-31 | 304.9 | 219.3 | 571 | — | — | — | — | — | 1309 | — | — |
| Ex-32 | 100.7 | 211.8 | 201.9 | 91.66 | 110.6 | 11060 | 11060 | 174.5 | — | 222.1 | 274 |
| Ex-33 | 13.64 | 28.69 | 35.68 | 35.34 | 19.7 | 11060 | 11060 | 60.11 | — | 126.2 | 52.9 |
| Ex-34 | 260.9 | 439.8 | 494 | 175.1 | 1092 | 11060 | 11060 | 899.9 | — | — | — |
| Ex-35 | 31.94 | 73.67 | 66.68 | 29.18 | 47.14 | 11060 | 11060 | 73.46 | 263.7 | 171.5 | 121.3 |
| Ex-36 | 163.7 | 532.9 | 330.2 | 124.8 | 149.6 | 11060 | 11060 | 267.5 | — | — | — |
| Ex-37 | 1449 | 1493 | 3069 | 1480 | 2448 | 11060 | 11060 | 1805 | — | — | — |
| Ex-38 | 96.62 | 186.4 | 239.5 | 36.01 | 123.3 | 11060 | 11060 | 121.7 | 395.3 | 277.2 | 196.9 |
| Ex-39 | 9.227 | 30.86 | 30.01 | 9.416 | 20.37 | 11060 | 11060 | 35.72 | 117.5 | 74.31 | 44.16 |
| Ex-40 | 61.5 | 83.38 | 137.7 | 34.41 | 52.42 | 11060 | 11060 | 113.8 | 294.2 | 124.5 | 158.8 |
| Ex-41 | 121.5 | 166.5 | 259.9 | — | — | — | — | — | 635.1 | — | — |
| Ex-42 | 135 | 144.3 | 286.1 | — | — | — | — | — | 1376 | — | — |
| Ex-43 | 13.89 | 26.82 | 44.36 | 24.31 | 17.47 | 11060 | 11060 | — | 130.2 | 61.99 | 145 |
| Ex-44 | 141 | 242.1 | 281 | — | — | — | — | — | 452.3 | 280.7 | 372.8 |
| Ex-45 | 166.6 | 152.8 | 373.7 | — | — | — | — | — | 1107 | — | — |
| Ex-46 | 72.4 | 157.2 | 166.3 | — | — | — | — | — | 934.2 | — | — |
| Ex-47 | 31.99 | 54.96 | 101.3 | 17.88 | 32.58 | 11060 | 11060 | — | 385.3 | — | — |
| Ex-48 | 18.2 | 28.52 | 50.44 | — | — | — | — | — | 152.9 | 105.5 | 122.6 |
| Ex-49 | 39.56 | 59.26 | 74.14 | — | — | — | — | — | 1933 | — | — |
| Ex-50 | 242.5 | 291 | 326 | — | — | — | — | — | 3956 | — | — |
| Ex-51 | 114.9 | 140.2 | 233.6 | — | — | — | — | — | 1411 | — | — |
| Ex-52 | 4.046 | 12.89 | 11.87 | 30.06 | 9.015 | 11060 | 11060 | | 68.71 | 37.26 | 48.56 |
| Ex-53 | 26.36 | 46.75 | 91.18 | — | — | — | — | — | 464.6 | — | — |
| Ex-54 | 5.062 | 11.08 | 10.79 | — | — | — | — | — | 84.62 | 185.6 | 56.5 |
| Ex-55 | 9.341 | 18.23 | 26.35 | — | — | — | — | — | 107.1 | 45.88 | 44.96 |
| Ex-56 | 9.15 | 32.58 | 26.49 | 127.7 | 22.61 | 11060 | 11060 | — | 48.88 | 57.45 | 45.61 |
| Ex-57 | 26.89 | 41.27 | 29.35 | 62.52 | — | — | — | — | — | — | — |
| Ex-58 | 5.413 | 13.53 | 17.57 | 16.54 | 10.41 | 11060 | 11060 | — | 57.09 | 36.19 | 35.53 |
| Ex-59 | 71.91 | 125 | 112.1 | 182.6 | — | — | — | — | — | — | — |
| Ex-60 | 9.46 | 18.59 | 29.03 | 22.36 | 15.84 | 11060 | 11060 | — | 99.48 | 41.88 | 44.48 |
| Ex-61 | 127 | 235.8 | 230.9 | — | — | — | — | — | 1278 | — | — |
| Ex-62 | 29.78 | 21.71 | 53.72 | — | — | — | — | — | 174.9 | 130.9 | 251.1 |
| Ex-63 | 19.59 | 39.32 | 55.24 | — | — | — | — | — | 184.9 | 121.1 | 113.8 |
| Ex-64 | 99.13 | 162.7 | 250.1 | — | — | — | — | — | 485.9 | 294 | 389.3 |
| Ex-65 | 69.3 | 65.27 | 183.9 | 362.1 | 172.1 | 11060 | 11060 | — | 560.4 | — | — |
| Ex-66 | 93.59 | 57.91 | 218.4 | — | — | — | — | — | 456.9 | — | — |
| Ex-67 | 11.87 | 23.08 | 40.02 | — | — | — | — | — | 113.4 | 322.7 | 186.2 |
| Ex-68 | 21.9 | 97.18 | 57.25 | 39 | — | — | — | — | — | — | — |
| Ex-69 | 6.79 | 10.19 | 28.65 | 20.35 | 12.51 | 11060 | 11060 | — | 68.54 | 51.86 | 52.66 |
| Ex-70 | 6.495 | 19.59 | 33.79 | — | — | — | — | — | 95.12 | 29.59 | 61.77 |
| Ex-71 | 4.003 | 11.54 | 17.58 | — | — | — | — | — | 66.36 | 150.4 | 50.2 |
| Ex-72 | 15.04 | 35.3 | 53.88 | — | — | — | — | — | 139 | 53.75 | 82.37 |
| Ex-73 | 9.667 | 52.24 | 60.44 | — | — | — | — | — | 1058 | — | — |
| Ex-74 | 437.8 | 1262 | 933.1 | 818.6 | — | — | — | — | — | — | — |
| Ex-75 | 19.35 | 117.6 | 60.8 | 55.99 | — | — | — | — | — | — | — |
| Ex-76 | 5.463 | 20.42 | 8.471 | 55.76 | 7.219 | 11060 | 11060 | — | 24.14 | 18.47 | 28.14 |
| Ex-77 | 14.97 | 20.79 | 61.81 | — | — | — | — | — | 175 | — | — |
| Ex-78 | 94.7 | 84.77 | 194 | — | — | — | — | — | 433.6 | — | — |
| Ex-79 | 5.837 | 10.85 | 8.199 | 57.9 | 27.93 | 11060 | 11060 | — | 23.23 | 8.941 | 16.17 |
| Ex-80 | 32.74 | 42.04 | 88.96 | — | — | — | — | — | 200.1 | 131.3 | 143.1 |
| Ex-81 | 85.64 | 86.25 | 164.4 | — | — | — | — | — | 1110 | — | — |
| Ex-82 | 31.2 | 32.77 | 54.65 | — | — | — | — | — | 591.7 | — | — |
| Ex-83 | 80.08 | 102.8 | 129.8 | — | — | — | — | — | 1364 | — | — |
| Ex-84 | 670.2 | 846.8 | 1382 | — | — | — | — | — | 2889 | — | — |
| Ex-85 | 38.79 | 44.32 | 81.09 | — | — | — | — | — | 764 | — | — |
| Ex-86 | 14.57 | 21.24 | 33.77 | — | — | — | — | — | 361.2 | — | — |
| Ex-87 | 41.59 | 31.43 | 80.03 | — | — | — | — | — | 176.2 | 96.58 | 108.7 |
| Ex-88 | 18.47 | 42.47 | 37.17 | — | — | — | — | — | 159.1 | 36.09 | 121.6 |
| Ex-89 | 79.99 | 244.6 | 170.9 | 138.3 | — | — | — | — | — | — | — |
| Ex-90 | 48.81 | 84.96 | 151.9 | — | — | — | — | — | 311.2 | 192.8 | 174.1 |
| Ex-91 | 64.95 | 102.6 | 129.3 | — | — | — | — | — | 399.3 | — | — |
| Ex-92 | 225.8 | 340.3 | 482.2 | — | — | — | — | — | 1860 | — | — |
| Ex-93 | 1.644 | 3.699 | 3.13 | 7.029 | — | — | — | — | — | — | — |
| Ex-94 | 1.677 | 4.201 | 3.823 | 5.33 | — | — | — | — | — | — | — |
| Ex-95 | 49.82 | 64.95 | 99.42 | 91.55 | — | — | — | — | — | — | — |
| Ex-96 | 43.8 | 104.5 | 124.9 | 192.6 | — | — | — | — | — | — | — |
| Ex-97 | 3.164 | 7.193 | 8.375 | 9.726 | — | — | — | — | — | — | — |
| Ex-98 | 18.93 | 20.8 | 61.29 | — | — | — | — | — | 175 | 68.84 | 72.79 |
| Ex-99 | 27.33 | 65.61 | 56.53 | 52.44 | — | — | — | — | — | — | — |
| Ex-100 | 216.3 | 507.4 | 353.7 | 663.5 | — | — | — | — | — | — | — |
| Ex-101 | 1.989 | 5.164 | 4.001 | 6.437 | — | — | — | — | — | — | — |
| Ex-102 | 3.56 | 10.39 | 7.842 | 10.33 | — | — | — | — | — | — | — |
| Ex-103 | 2.117 | 8.722 | 6.28 | 18.15 | 4.514 | 8011 | 11060 | — | 26.27 | 19.87 | 30.88 |
| Ex-104 | 43.9 | 59.98 | 71.52 | 168.1 | — | — | — | — | — | — | — |
| Ex-105 | 26.71 | 37.23 | 72.22 | — | — | — | — | — | 158.3 | 80.52 | 136.1 |
| Ex-106 | 19.71 | 30.59 | 55.14 | — | — | — | — | — | 128 | — | — |
| Ex-107 | 3.814 | 9.518 | 6.198 | 19.75 | — | — | — | — | — | — | — |
| Ex-108 | 1.52 | 5.41 | 3.09 | — | — | — | — | — | 50.04 | 20.88 | 28.94 |
| Ex-109 | 4.331 | 5.478 | 6.124 | 34.18 | 4.728 | 8060 | 11060 | — | 38.38 | 18.83 | 15.54 |
| Ex-110 | 37.68 | 43.02 | 47.51 | 57.67 | — | — | — | — | — | — | — |
| Ex-111 | 18.68 | 38.13 | 34.12 | 44.38 | — | — | — | — | — | — | — |
| Ex-112 | 273.5 | 402.4 | 472.6 | 518.2 | — | — | — | — | — | — | — |
| Ex-113 | 88.46 | 123.4 | 149.8 | 176.5 | — | — | — | — | — | — | — |
| Ex-114 | 165.1 | 278 | 380 | — | — | — | — | — | 670.2 | — | — |
| Ex-115 | 3.984 | 13.25 | 10.79 | — | — | — | — | — | 46.63 | 18.92 | 24.58 |
| Ex-116 | 4.874 | 17.92 | 9.841 | — | — | — | — | — | 56.59 | 36.38 | 46.42 |
| Ex-117 | 73.57 | 96.1 | 218.1 | — | — | — | — | — | 719.5 | — | — |
| Ex-118 | 149.6 | 123.4 | 432.2 | — | — | — | — | — | 1060 | — | — |
| Ex-119 | 229.7 | 156.6 | 662.1 | — | — | — | — | — | 2154 | — | — |
| Ex-120 | 402.5 | 268.8 | 1079 | — | — | — | — | — | 2030 | — | — |
| Ex-121 | 369 | 278.3 | 1033 | — | — | — | — | — | 3426 | — | — |
| Ex-122 | 403.5 | 280.4 | 907.9 | — | — | — | — | — | — | — | — |
| Ex-123 | 173.5 | 157.1 | 434.4 | — | — | — | — | — | 2064 | — | — |
| Ex-124 | 11.08 | 24.05 | 39.36 | — | — | — | — | — | 151 | — | — |
| Ex-125 | 66.03 | 94 | 161.2 | — | — | — | — | — | 1032 | — | — |
| Ex-126 | 34.88 | 52.79 | 88.33 | 30.71 | 58.56 | 11060 | 11060 | 85.4 | 271.2 | 319.6 | 169 |
| Ex-127 | 103.2 | 121.5 | 233.2 | 91.71 | 287.3 | 11060 | 11060 | 351.1 | — | 482.7 | 509.5 |
| Ex-128 | 96.91 | 143.2 | 245.6 | 102.4 | 204.3 | 11060 | 11060 | 242.4 | 666.9 | — | 214.4 |
| Ex-129 | 56.2 | 85.03 | 164.8 | 50.03 | 79.05 | 11060 | 11060 | 144.5 | 468.8 | 210.9 | 241.4 |
| Ex-130 | 20.25 | 77.21 | 114.8 | 100.7 | 75.16 | 4920 | 11060 | 27.74 | 68.65 | 115.6 | 248.4 |
| Ex-131 | 3.574 | 18.22 | 11.32 | 97.89 | 7.213 | 388.7 | 5161 | — | 21.83 | 15.1 | 22.79 |
| Ex-132 | 5.454 | 13.9 | 15.65 | — | — | — | — | — | 24.04 | 14.53 | 44.43 |
| Ex-133 | 119.5 | 130.1 | 249.5 | 47.13 | 110.4 | 11060 | 11060 | 279.1 | — | — | — |
| Ex-134 | 1159 | 876.5 | 7211 | 647.9 | 2311 | 11060 | 11060 | 2679 | — | — | — |
| Ex-135 | 1.474 | 1.076 | 0.766 | 9.248 | 2.544 | 1566 | 9396 | 1.4 | 4.613 | 1.5 | 3.857 |
| Ex-136 | 21.61 | 40.56 | 60.01 | 38.98 | 24.78 | 11060 | 11060 | 58.4 | 130.7 | 253.2 | 118.2 |
| Ex-137 | 32.61 | 66.69 | 58 | 40.12 | — | — | — | — | — | — | — |
| Ex-138 | 150.1 | 244.9 | 398.4 | 119.6 | 336.8 | 11060 | 11060 | — | 702.8 | — | — |
| Ex-139 | 129.2 | 128.2 | 319.4 | 159.1 | 377.2 | 11060 | 11060 | — | 663 | — | — |
| Ex-140 | 69.3 | 65.27 | 183.9 | 362.1 | 172.1 | 11060 | 11060 | — | 560.4 | — | — |
| Ex-141 | 7.498 | 16.54 | 18.12 | 87.65 | 14.36 | 11060 | 11060 | — | 38.05 | 131.4 | 53.1 |
| Ex-142 | 19.19 | 29.29 | 49.18 | 34 | 31.09 | 11060 | 11060 | — | 82.06 | 96.71 | 119.6 |
| Ex-143 | 108.1 | 109.3 | 197.1 | 52.9 | 124.3 | 11060 | 11060 | — | 330.5 | | |
| Ex-144 | 6.196 | 14.31 | 14.55 | 45.96 | 10.74 | 11060 | 11060 | — | 41.7 | 73.05 | 53.25 |
| Ex-145 | 34.29 | 73.19 | 58.94 | 44.26 | 27.75 | 11060 | 11060 | — | 119.2 | 120.3 | 154.6 |
| Ex-146 | 15.32 | 22.08 | 58.24 | 77.6 | 29.36 | 11060 | 11060 | — | 152.8 | — | — |
| Ex-147 | 284.4 | 293.5 | 670 | 438 | 935.9 | 11060 | 11060 | — | 1262 | — | — |
| Ex-148 | 6.615 | 9.354 | 27.58 | 112.4 | 20.42 | 11060 | 11060 | — | 91.43 | — | — |
| Ex-149 | 1471 | 2371 | 1349 | 1921 | — | — | — | — | — | — | — |
| Ex-150 | 1207 | 2124 | 1866 | 742.6 | 1433 | 11060 | 11060 | 974.3 | | | |
| Ex-151 | 392.7 | 1177 | 690.3 | 571.7 | — | — | — | — | — | — | — |
| Ex-152 | 76.92 | 106.3 | 194.9 | — | — | — | — | — | 636.5 | — | — |
| Ex-153 | 122.9 | 214.9 | 321.8 | — | — | — | — | — | 1641 | — | — |
| Ex-154 | 104.4 | 103.2 | 276.7 | — | — | — | — | — | — | — | — |
| Ex-155 | 158.2 | 211.1 | 350.1 | — | — | — | — | — | 926.6 | — | — |
| Ex-156 | 5.213 | 16.71 | 18.86 | — | — | — | — | — | 76.13 | 45.22 | 113.4 |
| Ex-157 | 288.3 | 4235 | 9650 | — | — | — | — | — | 1309 | — | — |
| Ex-158 | 7.181 | 15.5 | 26.89 | 15.37 | — | — | — | — | — | — | — |
| Ex-159 | 259.9 | 117.7 | 657.6 | 48.34 | 328.2 | 11060 | 11060 | — | 1172 | — | — |
| Ex-160 | 2.434 | 5.247 | 4.413 | 53.21 | 4.555 | 5544 | 11060 | — | 18.91 | 7.873 | 19.9 |
| Ex-161 | 6.039 | 11.41 | 15.2 | — | — | — | — | — | 61.74 | 32.07 | 28.38 |
| Ex-162 | 19.79 | 47.51 | 43.62 | — | — | — | — | — | 132.6 | 48 | 105.7 |
| Ex-163 | 2.363 | 4.531 | 3.786 | 19.82 | 3.126 | 1452 | 7373 | — | 14.64 | 2.29 | 20.82 |
| Ex-164 | 1.487 | 3.148 | 2.055 | 35.09 | 6.606 | 1894 | 11060 | — | 5.762 | 2.764 | 14.61 |
| Ex-165 | 6.79 | 11.04 | 18.63 | 21.72 | 14.07 | 11060 | 11060 | — | 40.68 | 31.23 | 41.5 |
| Ex-166 | 6.648 | 15.81 | 12.45 | 17.99 | — | — | — | — | — | — | — |
| Ex-167 | 3.284 | 4.823 | 4.999 | 23.31 | 10.1 | 10280 | 11060 | — | 23.36 | 8.095 | 16.1 |
| Ex-168 | 3.173 | 5.488 | 7.252 | — | — | — | — | — | 31.42 | 12.99 | 25.79 |
| Ex-169 | 2.221 | 13.45 | 7.698 | 15.47 | 7.051 | 9789 | 11060 | — | 30.93 | 25.76 | 21.22 |
| Ex-170 | 32.86 | 33.37 | 89.6 | — | — | — | — | — | 219 | — | — |
| Ex-171 | 1.65 | 5.092 | 4.376 | 38.73 | 5.026 | 5129 | 11060 | — | 12.29 | 10.46 | 24.21 |
| Ex-172 | 34.1 | 49.22 | 72.42 | — | — | — | — | — | 252.1 | — | — |
| Ex-173 | 18.92 | 50.09 | 38.18 | — | — | — | — | — | 161 | 61.06 | 56.91 |
| Ex-174 | 23.63 | 26.18 | 63.84 | — | — | — | — | — | 254.4 | 91.1 | 73.59 |
| Ex-175 | 82.11 | 66.33 | 186.4 | — | — | — | — | — | 856.1 | 277.5 | 323.6 |
| Ex-176 | 6.221 | 11.55 | 19.84 | 10.54 | 12.55 | 11060 | 11060 | — | 51.91 | 22.67 | 44.8 |
| Ex-177 | 23.06 | 61.67 | 77.38 | — | — | — | — | — | 248.1 | — | — |
| Ex-178 | 905.9 | 656.8 | 1283 | 240.5 | 1062 | 11060 | 11060 | 1700 | — | — | — |
| Ex-179 | 7603 | 1596 | 2758 | 124.3 | 1320 | 11060 | 11060 | 3898 | — | — | — |
| Ex-180 | 2272 | 428.4 | 724.8 | 78.04 | 813.2 | 11060 | 11060 | 3932 | — | — | — |
| Ex-181 | 1320 | 757.9 | 1485 | 787 | — | — | — | — | — | — | — |
| Ex-182 | 3631 | 3198 | 2193 | 1262 | — | — | — | — | — | — | — |
| Ex-183 | 2187 | 4643 | 2987 | 2877 | — | — | — | — | — | — | — |
| Ex-184 | 590.6 | 193.9 | 822.2 | 96.72 | 668.1 | 11060 | 11060 | 811.7 | — | — | — |
| Ex-185 | 482.4 | 318.7 | 1369 | 145.8 | 842.4 | 11060 | 11060 | — | 4576 | — | — |
| Ex-186 | 409.2 | 435.5 | 979.9 | — | — | — | — | — | 2494 | — | — |
| Ex-187 | 389.9 | 170.4 | 498.7 | 72.55 | 223.1 | 11060 | 11060 | — | 3570 | — | — |
| Ex-188 | 4952 | 752.9 | 1859 | 561.5 | 1248 | 11060 | 11060 | — | 3662 | — | — |
| Ex-189 | 3012 | 2196 | 2247 | 1360 | — | — | — | — | — | — | — |
| Ex-190 | 438 | 490.4 | 432.3 | 340.5 | — | — | — | — | — | — | — |
| Ex-191 | 11110 | 5633 | 11110 | 422.9 | 3693 | 11060 | 11060 | — | 8563 | — | — |
| Ex-192 | 283.2 | 192.4 | 418.3 | 120.9 | 409 | 11060 | 11060 | — | 30000 | — | — |
| Ex-193 | 643.1 | 476.4 | 1198 | 418.3 | 970.1 | 11060 | 11060 | — | 18290 | — | — |
| Ex-194 | 57.6 | 61.31 | 167.1 | 55.09 | — | — | — | — | — | — | — |
| Ex-195 | 76.73 | 77.93 | 251.1 | — | — | — | — | — | 7557 | — | — |
| Ex-196 | 8.056 | 19.42 | 35.82 | — | — | — | — | — | 1874 | — | — |
| Ex-197 | 200.1 | 186.4 | 432 | — | — | — | — | — | 393.3 | 459.4 | 512.8 |
| Ex-198 | 36.02 | 33.55 | 131.5 | — | — | — | — | — | 140.2 | 74.27 | 120.5 |
| Ex-199 | 86.85 | 92.63 | 287 | — | — | — | — | — | 374.2 | 190 | 312.6 |
| Ex-200 | 141.2 | 92.9 | 474.1 | — | — | — | — | — | 612.7 | — | — |
| Ex-201 | 284.7 | 205.5 | 403.9 | — | — | — | — | — | 923 | 219.6 | 372.7 |
| Ex-202 | 97.49 | 229.4 | 292.5 | — | — | — | — | — | 701.2 | — | — |
| Ex-203 | 493.4 | 936 | 767.4 | 590.4 | — | — | — | — | — | — | — |
| Ex-204 | 339.2 | 238.9 | 935.9 | — | — | — | — | — | 2314 | — | — |
| Ex-205 | 1.692 | 4.421 | 3.944 | 40.08 | 10.65 | 5092 | 11060 | — | 22.28 | 15.14 | 16.97 |
| Ex-206 | 7.07 | 22.25 | 23.5 | 3.568 | — | — | — | — | 24 | 15.36 | 22.87 |
| Ex-208 | 2.438 | 11.12 | 5.789 | 44.51 | 20.19 | 11060 | 11060 | — | 20.39 | 17.03 | 63.64 |
| Ex-209 | 2.91 | 11.99 | 9.463 | 47.72 | 13.59 | 9435 | 11060 | — | 53.04 | 49.74 | 158.3 |
| Ex-210 | 4.71 | 14.14 | 12.21 | 13.39 | 12.67 | 11060 | 11060 | — | 34.96 | 12.31 | 20.07 |
| Ex-211 | 5.846 | 12.14 | 16.5 | 45.35 | 15.04 | 11060 | 11060 | — | 56.88 | 49.42 | 82.97 |
| Ex-212 | 4.752 | 39.8 | 17.82 | — | — | — | — | — | 67.3 | 56.07 | 89.78 |
| Ex-213 | 3.503 | 11.25 | 12.22 | — | — | — | — | — | 48.3 | 42.87 | 66.44 |
| Ex-214 | 62.78 | 242.8 | 498.1 | — | — | — | — | — | 135.6 | 106.8 | 171.3 |
| Ex-215 | 3.747 | 20.88 | 16.04 | 105.1 | 38.86 | 1608 | 11060 | — | 55.93 | 23.01 | 32.6 |
| Ex-216 | 463.6 | 863.5 | 1587 | — | — | — | — | — | 2901 | — | — |
| Ex-217 | 3.462 | 13.07 | 8.852 | — | — | — | — | — | 47.71 | 10.88 | 46.89 |
| Ex-218 | 3.425 | 8.692 | 6.762 | 77.42 | 7.465 | 11060 | 11060 | — | 31.9 | 20.23 | 37.36 |
| Ex-219 | 82.69 | 196.2 | 254.8 | — | — | — | — | — | 304.7 | 229.8 | 318.5 |
| Ex-220 | 27.82 | 70.56 | 72.35 | — | — | — | — | — | 175.7 | 129.7 | 170.7 |
| Ex-221 | 5.549 | 21.18 | 22.82 | — | — | — | — | — | 175.2 | 79.39 | 80.71 |
| Ex-222 | 29.3 | 46.03 | 104.3 | — | — | — | — | — | 245.8 | — | — |
| Ex-223 | 9.265 | 34.07 | 24.46 | 111.1 | 37.62 | 11060 | 11060 | — | 63.77 | 23.56 | 42.88 |
| Ex-224 | 2.718 | 11.9 | 5.373 | 18.99 | 5.483 | 3306 | 11060 | — | 19.37 | 9.815 | 23.48 |
| Ex-225 | 12.68 | 21.51 | 39.92 | — | — | — | — | — | — | — | — |
| Ex-226 | 30.57 | 42.12 | 50.93 | 48.02 | — | — | — | — | — | — | — |
| Ex-227 | 6.564 | 21.94 | 18.48 | — | — | — | — | — | 175.1 | — | — |
| Ex-228 | 16.05 | 38.23 | 53 | — | — | — | — | — | 379.3 | 132.9 | 180.2 |
| Ex-229 | 6.535 | 19.08 | 22.46 | — | — | — | — | — | 109.4 | 38.21 | 51.93 |
| Ex-230 | 16.05 | 70.11 | 88.49 | 21.11 | 13.85 | 1640 | 11060 | — | 51.53 | 23.21 | 52.72 |
| Ex-231 | 2.874 | 8.766 | 8.547 | 20.34 | 15.01 | 492.5 | 11060 | — | 25.68 | — | — |
| Ex-232 | 103.6 | 197 | 761.5 | — | — | — | — | — | 188 | 117.4 | 84.22 |
| Ex-233 | 10.07 | 25.86 | 153.1 | — | — | — | — | — | 497.7 | — | — |
| Ex-234 | 27.37 | 106.7 | 388.9 | — | — | — | — | — | 1519 | — | — |
| Ex-235 | 3.865 | 19.4 | 31.62 | — | — | — | — | — | 206.5 | — | — |
| Ex-236 | 16.25 | 34.83 | 121.1 | — | — | — | — | — | 242.3 | — | — |
| Ex-237 | 24.88 | 32.93 | 158.4 | — | — | — | — | — | 292.8 | — | — |
| Ex-238 | 112.3 | 399.9 | 446.5 | — | — | — | — | — | 4122 | — | — |
| Ex-239 | 1.849 | 11.43 | 8.198 | — | — | — | — | — | 54.55 | 29.41 | 33.74 |
| Ex-240 | 4.311 | 10.4 | 20.04 | 19.44 | 45.83 | 2220 | 11060 | — | 58.81 | 36.13 | 39.32 |
| Ex-241 | 98.21 | 307.6 | 1859 | — | — | — | — | — | 1408 | — | — |
| Ex-242 | 90.66 | 347.7 | 694.3 | — | — | — | — | — | 1150 | — | — |
| Ex-243 | 87.32 | 332.8 | 985.8 | — | — | — | — | — | 943.2 | — | — |
| Ex-244 | 140.1 | 206.6 | 698.6 | — | — | — | — | — | 711.3 | — | — |
| Ex-245 | 4.216 | 12.9 | 18.46 | 19.09 | 15.85 | 1293 | 11060 | — | 70.39 | 47.64 | 44.05 |
| Ex-246 | 96.76 | 275.1 | 373.7 | — | — | — | — | — | 1167 | — | — |
| Ex-247 | 74.57 | 320.4 | 352.4 | — | — | — | — | — | 976.8 | — | — |
| Ex-248 | 3.089 | 15.54 | 12.54 | — | — | — | — | — | 45.73 | 42.81 | 36.27 |
| Ex-249 | 2.694 | 15.46 | 20.22 | 11.42 | 21.32 | 943.2 | 11060 | — | 73.83 | 51.2 | 36.49 |
| Ex-250 | 8.558 | 18.56 | 36.22 | — | — | — | — | — | 151.6 | — | — |
| Ex-251 | 52.98 | 109.5 | 488.7 | — | — | — | — | — | 670.2 | — | — |
| Ex-252 | 4.96 | 32.23 | 57.76 | — | — | — | — | — | 231.5 | — | — |
| Ex-253 | 4.096 | 15.87 | 20.08 | 15.18 | 18.41 | 1065 | 11060 | — | 57.58 | 49.56 | 49.7 |
| Ex-254 | 6.964 | 91.87 | 59.65 | — | — | — | — | — | 314.8 | — | — |
| Ex-255 | 4.791 | 14.92 | 18.52 | 86.66 | 12.84 | 1389 | 11060 | — | 93.27 | 40.14 | 25.34 |
| Ex-256 | 4.408 | 11.06 | 16.52 | — | — | — | — | — | 126.3 | — | — |
| Ex-257 | 4.425 | 28.69 | 42.48 | — | — | — | — | — | 144.2 | — | — |
| Ex-258 | 7.003 | 25.17 | 38.79 | — | — | — | — | — | 123.8 | — | — |
| Ex-259 | 11.31 | 34.22 | 79.74 | — | — | — | — | — | 184.1 | — | — |
| Ex-260 | 41.56 | 184.7 | 486 | — | — | — | — | — | 1214 | — | — |
| Ex-261 | 16.96 | 58.27 | 81.56 | — | — | — | — | — | 368.8 | — | — |
| Ex-262 | 4.437 | 26.75 | 22.21 | — | — | — | — | — | 118.6 | 66.13 | 52.36 |
| Ex-263 | 3.025 | 12.54 | 11.36 | — | — | — | — | — | 111.4 | — | — |
| Ex-264 | 63.93 | 214.8 | 350.9 | — | — | — | — | — | 618.5 | — | — |
| Ex-265 | 4.214 | 14.95 | 24.47 | — | — | — | — | — | 200.1 | — | — |
| Ex-266 | 3.748 | 21.62 | 23.35 | 68.84 | 28.17 | 1294 | 11060 | — | 89.66 | 39.25 | 47.27 |
| Ex-267 | 3.261 | 8.109 | 5.561 | 23.46 | 9.935 | 4249 | 11060 | — | 23.63 | 24.04 | 18.74 |
| Ex-268 | 2.47 | 5.787 | 5.724 | 87.49 | 10.3 | 4189 | 11060 | — | 55.98 | 27.55 | 30.66 |
| Ex-269 | 8.129 | 19.97 | 24.89 | — | — | — | — | — | 181.8 | 84.51 | 90.08 |
| Ex-270 | 8.78 | 30.92 | 27.94 | — | — | — | — | — | 142 | — | — |
| Ex-271 | 42.2 | 37.34 | 78.77 | — | — | — | — | — | 244.2 | — | — |
| Ex-272 | 3.185 | 19.36 | 29.35 | — | — | — | — | — | 62.36 | 29.45 | 50.53 |
| Ex-273 | 359.9 | 573.7 | 1226 | 455.9 | 1099 | 11060 | 11060 | 561.5 | — | — | — |
| Ex-274 | 671.3 | 400.8 | 330.5 | 120.6 | 87.56 | 11060 | 11060 | 1009 | — | — | — |
| Ex-275 | 1308 | 2950 | 1524 | 2217 | 1197 | 11060 | 11060 | — | — | — | — |
| Ex-276 | 422.6 | 312.1 | 796.3 | 124.5 | 460.7 | 11060 | 11060 | — | 1810 | — | — |
| Ex-277 | 39.24 | 64.64 | 94.62 | 183.9 | 37.99 | 11060 | 11060 | — | 1098 | — | — |
| Ex-278 | 14.64 | 30.37 | 45.96 | 74.91 | 20.34 | 11060 | 11060 | — | 364.2 | 103 | 98.22 |
| Ex-279 | 5.152 | 16.03 | 13.55 | 97.58 | 7.593 | 11060 | 11060 | — | 113.6 | 99.75 | 42.95 |
| Ex-280 | 4.548 | 12.43 | 9.466 | 61.05 | 4.888 | 8662 | 11060 | — | 78.24 | 106.3 | 62.01 |
| Ex-281 | 8.239 | 11.69 | 31.15 | — | — | — | — | — | 355.5 | — | — |
| Ex-282 | 4.26 | 12.52 | 13.51 | 5.844 | — | — | — | — | — | — | — |
| Ex-283 | 5.257 | 15.4 | 14.5 | — | — | — | — | — | 244 | 41.48 | 77.43 |
| Ex-284 | 194.7 | 137.6 | 577.7 | — | — | — | — | — | 934.6 | — | — |
| Ex-285 | 322.4 | 999.5 | 889.4 | 644.8 | — | — | — | — | — | — | — |
| Ex-286 | 204.8 | 50.28 | 52.02 | — | — | — | — | — | 1106 | — | — |
| Ex-287 | 5.277 | 15.85 | 10.15 | — | — | — | — | — | 41.49 | — | — |
| Ex-288 | 34.59 | 25.9 | 69.15 | 22.01 | 50.72 | 11060 | 11060 | — | 374.1 | — | — |
| Ex-289 | 6.968 | 26.94 | 22.31 | — | — | — | — | — | 103 | 64.6 | 81.39 |
| Ex-290 | 611.4 | 364.8 | 1398 | 175.4 | 1028 | 11060 | 11060 | 2327 | — | — | — |
| Ex-291 | 153.1 | 206.9 | 377 | 115.6 | 210.5 | 11060 | 11060 | 306.5 | — | — | — |
| Ex-292 | 175.4 | 133.6 | 603.5 | 76.86 | 308.3 | 11060 | 11060 | — | 2316 | — | — |
| Ex-293 | 148.8 | 203.3 | 455.2 | 136.1 | 296.2 | 11060 | 11060 | — | 1008 | — | — |
| Ex-294 | 113 | 125.3 | 261.6 | 30.11 | 105.7 | 11060 | 11060 | — | 294.9 | — | — |
| Ex-295 | 72.12 | 98.19 | 185.4 | — | — | — | — | — | 1897 | — | — |
| Ex-296 | 48.71 | 100.8 | 166.6 | 43.87 | 80.12 | 11060 | 11060 | — | 315.9 | — | — |
| Ex-297 | 23.05 | 57.62 | 67.81 | — | — | — | — | — | 635.5 | — | — |
| Ex-298 | 49.78 | 159.4 | 112.8 | 85.16 | — | — | — | — | — | — | — |
| Ex-299 | 115.4 | 93.06 | 293.1 | — | — | — | — | — | 1452 | — | — |
| Ex-300 | 6.526 | 8.432 | 14.4 | — | — | — | — | — | 222.1 | 39.83 | 54.58 |
| Ex-301 | 49.45 | 86.09 | 107.3 | 106.8 | — | — | — | — | — | — | — |
| Ex-302 | 8.989 | 17 | 18.62 | — | — | — | — | — | 135 | 48.88 | 49.53 |
| Ex-303 | 379.6 | 837.5 | 732.4 | 1139 | — | — | — | — | — | — | — |
| Ex-304 | 13.1 | 33.86 | 33.99 | 24.87 | — | — | — | — | — | — | — |
| Ex-305 | 11.61 | 23.81 | 37.03 | — | — | — | — | — | 212 | 54.7 | 93.01 |
| Ex-306 | 4.699 | 18.91 | 16.63 | 10.82 | — | — | — | — | — | — | — |
| Ex-307 | 8.964 | 24.67 | 22.4 | — | — | — | — | — | 258.3 | — | — |

For Example Nos. 57, 59, 68, 74, 75, 89, 93-97, 99-102, 104, 107, 110-113, 137, 149, 151, 158, 166, 181-183, 189, 190, 194, 203, 207, 226, 282, 285, 298, 301, 303, 304, and 306 nucleotide exchange assays for KRAS G12D, WT, G12C, and G12V were performed according to Procedure B. All other compounds of the nucleotide exchange assays were tested according to Procedure A.

SEQUENCES (Procedure A)
SEQ ID NO: 1-Recombinant Human KRAS G12C
GLNDIFEAQKIEWHETEYKLVVVGACGVGKSALTIQLIQNHFVDEYDPTIED
SYRKQVVIDGETSLLDILDTAGQEEYSAMRDQYMRTGEGFLLVFAINNTKSFEDIHH
YREQIKRVKDSEDVPMVLVGNKSDLPSRTVDTKQAQDLARSYGIPFIETSAKTRQGV
DDAFYTLVREIRKHKEK
SEQ ID NO: 2-Recombinant Human KRAS G12D
GLNDIFEAQKIEWHETEYKLVVVGADGVGKSALTIQLIQNHFVDEYDPTIED
SYRKQVVIDGETCLLDILDTAGQEEYSAMRDQYMRTGEGFLCVFAINNTKSFEDIHH
YREQIKRVKDSEDVPMVLVGNKCDLPSRTVDTKQAQDLARSYGIPFIETSAKTRQGV
DDAFYTLVREIRKHKEK
SEQ ID NO: 3-Recombinant Human KRAS G12V
GLNDIFEAQKIEWHETEYKLVVVGAVGVGKSALTIQLIQNHFVDEYDPTIED
SYRKQVVIDGETCLLDILDTAGQEEYSAMRDQYMRTGEGFLCVFAINNTKSFEDIHH
YREQIKRVKDSEDVPMVLVGNKCDLPSRTVDTKQAQDLARSYGIPFIETSAKTRQGV
DDAFYTLVREIRKHKEK
SEQ ID NO: 4-Recombinant Human KRAS G13D
GLNDIFEAQKIEWHETEYKLVVVGAGDVGKSALTIQLIQNHFVDEYDPTIED
SYRKQVVIDGETCLLDILDTAGQEEYSAMRDQYMRTGEGFLCVFAINNTKSFEDIHH
YREQIKRVKDSEDVPMVLVGNKCDLPSRTVDTKQAQDLARSYGIPFIETSAKTRQGV
DDAFYTLVREIRKHKEK
SEQ ID NO: 5-Recombinant Human HRAS
GGGGSHMTEYKLVVVGAGGVGKSALTIQLIQNHFVDEYDPTIEDSYRKQV
VIDGETCLLDILDTAGQEEYSAMRDQYMRTGEGFLCVFAINNTKSFEDIHQYREQIKR
VKDSDDVPMVLVGNKCDLAARTVESRQAQDLARSYGIPYIETSAKTRQGVEDAFYT
LVREIRQH
SEQ ID NO: 6-Recombinant Human NRAS
GGGGMTEYKLVVVGAGGVGKSALTIQLIQNHFVDEYDPTIEDSYRKQVVID
GETCLLDILDTAGQEEYSAMRDQYMRTGEGFLCVFAINNSKSFADINLYREQIKRVK
DSDDVPMVLVGNKCDLPTRTVDTKQAHELAKSYGIPFIETSAKTRQGVEDAFYTLVR
EIRQYRMKKLN
SEQ ID NO: 7-Recombinant Human SOS Protein
MGSSHHHHHHSGENLYFQGSSGLNDIFEAQKIEWHESSEEQMRLPSADVYR
FAEPDSEENIIFEENMQPKAGIPIIKAGTVIKLIERLTYHMYADPNFVRTFLTTYRSFCK
PQELLSLIIERFEIPEPEPTEADRIAIENGDQPLSAELKRFRKEYIQPVQLRVLNVCRHW
VEHHFYDFERDAYLLQRMEEFIGTVRGKAMKKWVESITKIIQRKKIARDNGPGHNIT
FQSSPPTVEWHISRPGHIETFDLLTLHPIEIARQLTLLESDLYRAVQPSELVGSVWTKE
DKEINSPNLLKMIRHTTNLTLWFEKCIVETENLEERVAVVSRIIEILQVFQELNNENGV
LEVVSAMNSSPVYRLDHTFEQIPSRQKKILEEAHELSEDHYKKYLAKLRSINPPCVPF
FGIYLTNILKTEEGNPEVLKRHGKELINFSKRRKVAEITGEIQQYQNQPYCLRVESDIK
RFFENLNPMGNSMEKEFTDYLFNKSLEIEPRNPKPLPRFPKKYSYPLKSPGVRPSNPRP
GT
SEQ ID NO: 8-Recombinant Human KRAS WT
GGGGTEYKLVVVGAGGVGKSALTIQLIQNHFVDEYDPTIEDSYRKQVVIDG
ETCLLDILDTAGQEEYSAMRDQYMRTGEGFLCVFAINNTKSFEDIHHYREQIKRVKD
SEDVPMVLVGNKCDLPSRTVDTKQAQDLARSYGIPFIETSAKTRQGVDDAFYTLVRE
IRKHKEK
SEQUENCES (Procedure B)
SEQ ID NO: 9-KRAS G12C (amino acids 1-169, N-terminal His-tag)
MASSHHHHHHSSENLYFQGMTEYKLVVVGACGVGKSALTIQLIQNHFVDE
YDPTIEDSYRKQVVIDGETCLLDILDTAGQEEYSAMRDQYMRTGEGFLCVFAINNTK
SFEDIHHYREQIKRVKDSEDVPMVLVGNKCDLPSRTVDTKQAQDLARSYGIPFIETSA
KTRQGVDDAFYTLVREIRKHKEK
SEQ ID NO: 10-KRAS G12D (amino acids 1-169, N-terminal His-tag)
MASSHHHHHHSSENLYFQGMTEYKLVVVGADGVGKSALTIQLIQNHFVDE
YDPTIEDSYRKQVVIDGETCLLDILDTAGQEEYSAMRDQYMRTGEGFLCVFAINNTK
SFEDIHHYREQIKRVKDSEDVPMVLVGNKCDLPSRTVDTKQAQDLARSYGIPFIETSA
KTRQGVDDAFYTLVREIRKHKEK
SEQ ID NO: 11-KRAS G12V (amino acids 1-169, N-terminal His-tag)
MGSSHHHHHHSSGLVPRGSHMASMTGGQQMGRGSENLYFQGMTEYKLVV
VGAVGVGKSALTIQLIQNHFVDEYDPTIEDSYRKQVVIDGETCLLDILDTAGQEEYSA
MRDQYMRTGEGFLCVFAINNTKSFEDIHHYREQIKRVKDSEDVPMVLVGNKCDLPS
RTVDTKQAQDLARSYGIPFIETSAKTRQGVDDAFYTLVREIRKHKEK
SEQ ID NO: 12-KRAS WT (amino acids 1-169, N-terminal His-tag)
MASSHHHHHHSSENLYFQGMTEYKLVVVGAGGVGKSALTIQLIQNHFVDE
YDPTIEDSYRKQVVIDGETCLLDILDTAGQEEYSAMRDQYMRTGEGFLCVFAINNTK
SFEDIHHYREQIKRVKDSEDVPMVLVGNKCDLPSRTVDTKQAQDLARSYGIPFIETSA
KTRQGVDDAFYTLVREIRKHKEK
SEQ ID NO: 13-SOS1 (amino acids 564-1049, No tag)
GEEQMRLPSADVYRFAEPDSEENIIFEENMQPKAGIPIIKAGTVIKLIERLTYH
MYADPNFVRTFLTTYRSFCKPQELLSLIIERFEIPEPEPTEADRIAIENGDQPLSAELKR
FRKEYIQPVQLRVLNVCRHWVEHHFYDFERDAYLLQRMEEFIGTVRGKAMKKWVE
SITKIIQRKKIARDNGPGHNITFQSSPPTVEWHISRPGHIETFDLLTLHPIEIARQLTLLES
DLYRAVQPSELVGSVWTKEDKEINSPNLLKMIRHTTNLTLWFEKCIVETENLEERVA
VVSRIIEILQVFQELNNFNGVLEVVSAMNSSPVYRLDHTFEQIPSRQKKILEEAHELSE
DHYKKYLAKLRSINPPCVPFFGIYLTNILKTEEGNPEVLKRHGKELINFSKRRKVAEIT
GEIQQYQNQPYCLRVESDIKRFFENLNPMGNSMEKEFTDYLFNKSLEIEPRNPKPLPR
FPKKYSYPLKSPGVRPSNPRPGT

1. A compound of Formula (I) 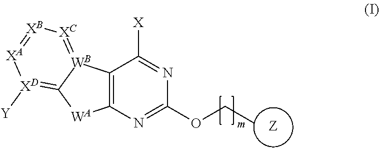
wherein:
X is: 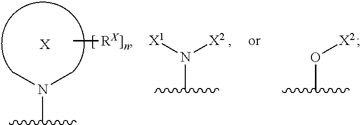
Ring X is selected from the group consisting of:
(i) a 5- to 9-membered monocyclic or fused bicyclic or bridged bicyclic heterocycloalkyl, wherein the 5- to 9-membered monocyclic or fused bicyclic or bridged bicyclic heterocycloalkyl is saturated and contains 0 to 2 heteroatom groups selected from the group consisting of N, S, S(O), S(O)₂ and O, in addition to the illustrated N atom; and
(ii) an 8- to 12-membered spiroheterocycloalkyl, wherein the 8- to 12-membered spiroheterocycloalkyl is saturated and contains 0 to 2 heteroatom groups selected from the group consisting of N, S, S(O), S(O)₂ and O, in addition to the illustrated N atom;
each R^{X} is independently selected from the group consisting of fluoro, cyano, hydroxy, oxo, C₁-C₆ alkyl, C₁-C₆ fluoroalkyl, C₁-C₆ alkoxy, C₁-C₆ fluoroalkoxy, C₁-C₆ cyanoalkyl, and C₁-C₆ hydroxyalkyl;
X¹ is selected from the group consisting of H, C₁-C₆ alkyl, C₁-C₆ fluoroalkyl, C₁-C₆ fluoroalkoxy, C₃-C₆ cycloalkyl, and C₃-C₆ fluorocycloalkyl;
X² is H or —(CR^{a}₂)ₚ—C^{X}, wherein C^{X} is:
(i) a 3- to 10-membered monocyclic or fused bicyclic or bridged bicyclic cycloalkyl or an 8- to 10-membered spirocycloalkyl;
(ii) a 3- to 10-membered monocyclic or fused bicyclic or bridged bicyclic heterocycloalkyl or an 8- to 10-membered spiroheterocycloalkyl, wherein the 3- to 10-membered monocyclic or fused bicyclic or bridged bicyclic heterocycloalkyl or the 8- to 10-membered spiroheterocycloalkyl is saturated and contains 1 to 3 heteroatom groups independently selected from the group consisting of N, S, S(O), S(O)₂ and O;
wherein C^{X} is unsubstituted or substituted by 1 to 2 R^{C}^{^{X}} substituents independently selected from the group consisting of fluoro, hydroxy, oxo, cyano, C₁-C₆ alkyl, C₁-C₆ acyl, C₁-C₆ fluoroalkyl, C₁-C₆ alkoxy, C₁-C₆ fluoroalkoxy, C₁-C₆ hydroxyalkyl, C₁-C₆ cyanoalkyl, C₃-C₆ cycloalkyl, and C₃-C₆ fluorocycloalkyl;
each R^{a} is independently selected from the group consisting of hydrogen, halo, cyano, hydroxy, C₁-C₆ alkyl, C₁-C₆ fluoroalkyl, C₁-C₆ alkoxy, C₁-C₆ fluoroalkoxy, C₁-C₆ cyanoalkyl, C₁-C₆ fluorocyanoalkyl, C₃-C₆ cycloalkyl, and C₃-C₆ fluorocycloalkyl;
X^{A}, X^{B}, and X^{C}, are independently selected from the group consisting of N, C(H), and C(R¹);
X^{D} is selected from the group consisting of N and C;
each R¹ is independently selected from the group consisting of C₁-C₃ alkyl, C₁-C₃ fluoroalkyl, C₁-C₃ alkoxy, C₁-C₃ fluoroalkoxy, halo, hydroxy, oxo, cyano, C₁-C₃ alkylamino, C₁-C₃ dialkylamino, and C^{B};
wherein C^{B} is selected from the group consisting of:
(i) a monocyclic, saturated heterocycloalkyl containing 1 to 3 heteroatoms independently selected from the group consisting of N, S, and O;
(ii) a monocyclic 5- to 6-membered heteroaryl containing 1 to 3 heteroatoms independently selected from the group consisting of N, S, and O;
wherein C^{B} is unsubstituted or substituted by 1 to 2 R^{C}^{^{B}} substituents independently selected from the group consisting of halo, hydroxy, oxo, cyano, C₁-C₃ alkyl, C₁-C₃ fluoroalkyl, C₁-C₃ alkoxy, C₁-C₃ fluoroalkoxy, C₃-C₆ cycloalkyl, and C₃-C₆ fluorocycloalkyl;
W^{A} is selected from the group consisting of N(R^{W1}), C(R^{W2})₂, O, S, Se, and C(R^{W3});
R^{W1} is selected from the group consisting of C₁-C₃ alkyl, C₁-C₃ fluoroalkyl, C₃-C₆ cycloalkyl, and C₃-C₆ fluorocycloalkyl;
each R^{W2} is independently selected from the group consisting of fluoro, C₁-C₃ alkyl, C₁-C₃ fluoroalkyl, and hydroxy;
or alternatively, the two R^{W2}, together with the carbon atom to which they are attached form a C₃-C₆ cycloalkyl or C₃-C₆ fluorocycloalkyl; R^{W3} is selected from the group consisting of H, halo, C₁-C₃ alkyl, C₁-C₃ alkoxy, C₁-C₃ fluoroalkyl, C₁-C₃ fluoroalkoxy, C₃-C₆ cycloalkyl, and C₃-C₆ fluorocycloalkyl;
W^{B} is C or N, wherein when:
W^{B} is C, then W^{A} is N(R^{W1}), C(R^{W2})₂, O, or S; and
W^{B} is N, then W^{A} is C(R^{W3});
Y is selected from the group consisting of C₁-C₆ alkyl, C₁-C₆ fluoroalkyl, and C^{Y};
C^{Y} is:
(i) an aryl selected from the group consisting of phenyl and naphthyl:
(ii) an indanyl or fused indanyl group of the formula: 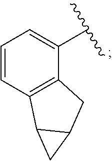 [SMILES: *c1cccc2c1CC1CC21]
(iii) a 5- to 6-membered monocyclic heteroaryl, wherein the 5- to 6-membered monocyclic heteroaryl contains 1 to 3 heteroatoms independently selected from the group consisting of N, S, and O; or
(iv) a 9- to 10-membered fused, bridged or spiro-bicyclic heteroaryl, wherein the 9- to 10-membered fused, bridged or spiro-bicyclic heteroaryl contains 1 to 3 heteroatoms independently selected from the group consisting of N, O, and S; or
(v) a 12- to 17-membered fused, bridged or spiro-tri- or tetracyclic heterocycloalkyl, where at least 2 of the rings of the 12- to 17-membered fused, bridged or spiro-tri- or tetracyclic heterocycloalkyl are aromatic, the third ring is partially unsaturated or aromatic, and the fourth ring, if present, is saturated, wherein the 12- to 17-membered fused, bridged or spiro-tri- or tetracyclic heterocycloalkyl contains 1 to 4 heteroatoms independently selected from the group consisting of N, S, and O;
wherein C^{Y} is unsubstituted or substituted by 1 to 5 RY substituents independently selected from the group consisting of halo, hydroxy, oxo, cyano, C₁-C₆ alkyl, C₂-C₆ alkynyl, C₂-C₇ alkenyl, C₁-C₆ fluoroalkyl, C₁-C₆ alkoxy, C₁-C₆ haloalkoxy, C₂-C₆ fluoroalkynyl, C₂-C₇ fluoroalkenyl, C₁-C₃ fluoroalkenylenyl, C₁-C₆ alkylthio, C₁-C₆ fluoroalkylthio, amino, C₁-C₆ alkylamino, C₁-C₆ dialkylamino, tri (C₁-C₆ alkyl) silyl, cyano, C₁-C₆ cyanoalkyl, C₁-C₆ fluorocyanoalkyl, C₁-C₃ alkoxy C₁-C₃ alkyl, C₁-C₆ alkoxycarbonyl, C₁-C₆ acyl, methylenyl, and R^{ya};
wherein R^{ya} is:
(a) a 3- to 9-membered monocyclic or fused bicyclic or bridged bicyclic cycloalkyl;
(b) a 3- to 9-membered monocyclic or fused bicyclic or bridged bicyclic cycloalkenyl;
(c) a 5- to 6-membered heteroaryl containing 1 to 3 heteroatoms independently selected from the group consisting of N, S, and O; or
(d) a 4- to 7-membered saturated or partially saturated heterocycloalkyl containing 1 to 2 heteroatoms independently selected from the group of N, S, and O;
wherein R^{ya} is unsubstituted or substituted by 1 to 2 substituents independently selected from the group consisting of halo, hydroxy, cyano, C₁-C₃ alkyl, C₁-C₃ fluoroalkyl, C₃-C₆ cycloalkyl, C₃-C₆ fluorocycloalkyl, C₁-C₃ alkoxy, C₁-C₃ fluoroalkoxy, and C₁-C₃ cyanoalkyl;
Ring Z is selected from the group consisting of:
(i) a 3- to 10-membered monocyclic, fused, bridged or spiro-bicyclic, or fused, bridged or spiro-tricyclic heterocycloalkyl, wherein the 3- to 10-membered monocyclic, fused, bridged or spiro-bicyclic, or fused, bridged or spiro-tricyclic heterocycloalkyl is saturated or partially unsaturated and contains 1 to 3 heteroatoms independently selected from the group consisting of N, S, and O;
(ii) a 3- to 10-membered monocyclic, fused, bridged or spiro-bicyclic, or fused, bridged or spiro-tricyclic cycloalkyl, wherein the 3- to 10-membered monocyclic, fused, bridged or spiro-bicyclic, or fused, bridged or spiro-tricyclic cycloalkyl is saturated or partially unsaturated; and
(iii) 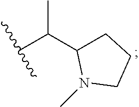 [SMILES: *C(C)C1CCCN1C]
wherein ring Z is unsubstituted or independently substituted with 1 to 4 substituents R^{ZC} selected from the group consisting of halo, hydroxy, oxo, cyano, C₁-C₃ alkyl, C₁-C₃ fluoroalkyl, C₁-C₃ hydroxyalkyl, C₁-C₃ fluoroalkenylenyl, C₁-C₃ hydroxyfluoroalkyl, C₁-C₃ alkoxy, C₁-C₃ fluoroalkoxy, C₁-C₃ cyanoalkyl, C₃-C₆ cycloalkyl, C₃-C₆ fluorocycloalkyl, C₃-C₆ hydroxycycloalkyl, C₃-C₆ hydroxyfluorocycloalkyl, C₂-C₄ fluoroalkenyl, C₁-C₃ alkylamino, C₁-C₃ dialkylamino, methylene (C₁-C₃ alkyl)amino, C₁-C₃ alkylenedi (C₁-C₃ alkyl)amino and methylene (C₁-C₃ alkyl) (C₁-C₃ alkyl) carbamate;
wherein ring Z is optionally substituted with 1 P or —CH₂P, wherein P is a 4- to 10-membered monocyclic, fused, bridged or spiro-bicyclic, or fused, bridged or spiro-tricyclic heterocycloalkyl, wherein the 4- to 10-membered monocyclic, fused, bridged or spiro-bicyclic, or fused, bridged or spiro-tricyclic heterocycloalkyl is saturated or partially unsaturated and contains 1 to 2 heteroatoms selected from the group consisting of N, S, and O;
wherein P is unsubstituted or independently substituted with 1 to 4 R^{P} substituents selected from the group consisting of halo, hydroxy, oxo, cyano, C₁-C₃ alkyl, C₁-C₃ fluoroalkyl, C₁-C₃ hydroxyalkyl, C₁-C₃ hydroxyfluoroalkyl, C₁-C₃ alkoxy, C₁-C₃ fluoroalkoxy, C₁-C₃ cyanoalkyl, C₃-C₆ cycloalkyl, C₃-C₆ fluorocycloalkyl, C₃-C₆ hydroxycycloalkyl, C₁-C₃ alkoxy C₁-C₃ alkyl, C₃-C₆ hydroxyfluorocycloalkyl, carbamoyl, and —NHC(O)C₁-C₃alkyl;
subscript m is 0, 1, or 2;
subscript n is 0, 1, 2, or 3; and
subscript p is 0, 1, 2, or 3;
or a pharmaceutically acceptable salt thereof.

2. The compound of claim 1 or the pharmaceutically acceptable salt thereof, wherein X is

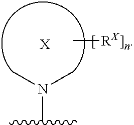

3. The compound of claim 2 or the pharmaceutically acceptable salt thereof, wherein X is

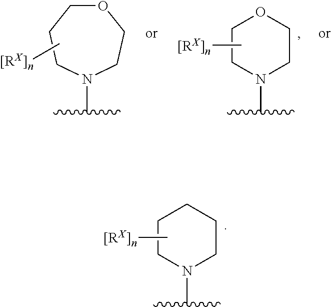

4. The compound of claim 1 or the pharmaceutically acceptable salt thereof, wherein X is

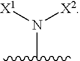

5. The compound of claim 4 or the pharmaceutically acceptable salt thereof, wherein X¹ is H and X² is —(CH₂)ₚ—C^{X}, wherein C^{X} is selected from the group consisting of: 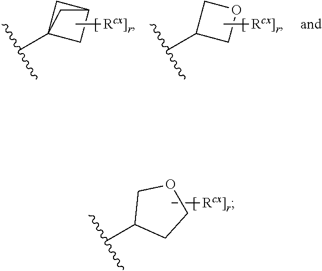
and
subscript r is 0, 1, or 2.

6.-12. (canceled)

13. The compound of claim 1 or the pharmaceutically acceptable salt thereof, wherein Y is C^{Y}, and C^{Y} is unsubstituted or substituted phenyl, naphthyl, pyridyl, indazolyl, benzothienyl, benzoxazolyl, benzothiazolyl, or isoquinolinyl.

14. (canceled)

15. The compound of claim 1 or the pharmaceutically acceptable salt thereof, wherein C^{Y} is selected from the group consisting of: 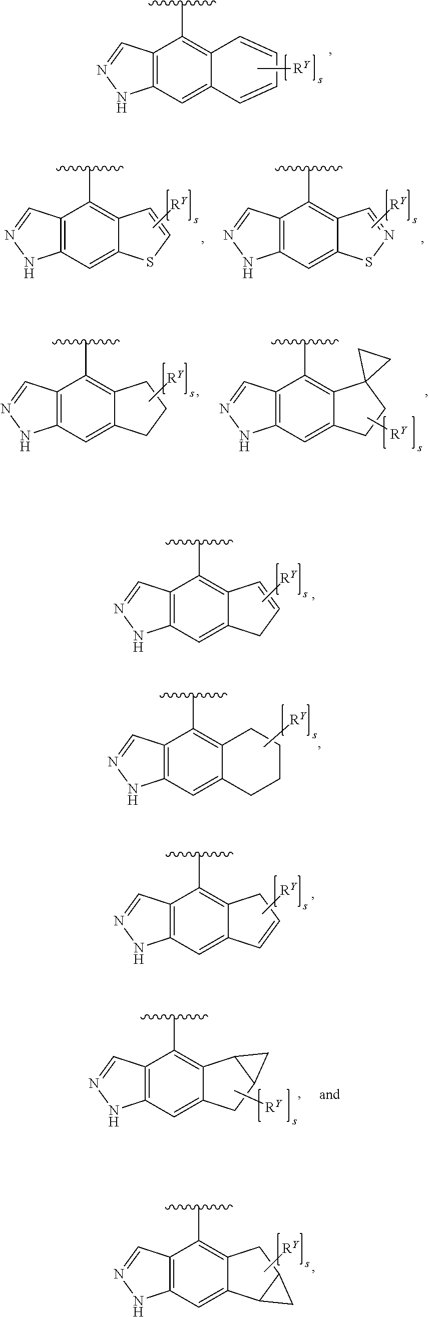
and subscript s is 0, 1, 2, 3, or 4.

16. (canceled)

17. The compound of claim 1 or the pharmaceutically acceptable salt thereof, wherein Y is C^{Y}, wherein C^{Y} is selected from the group consisting of:

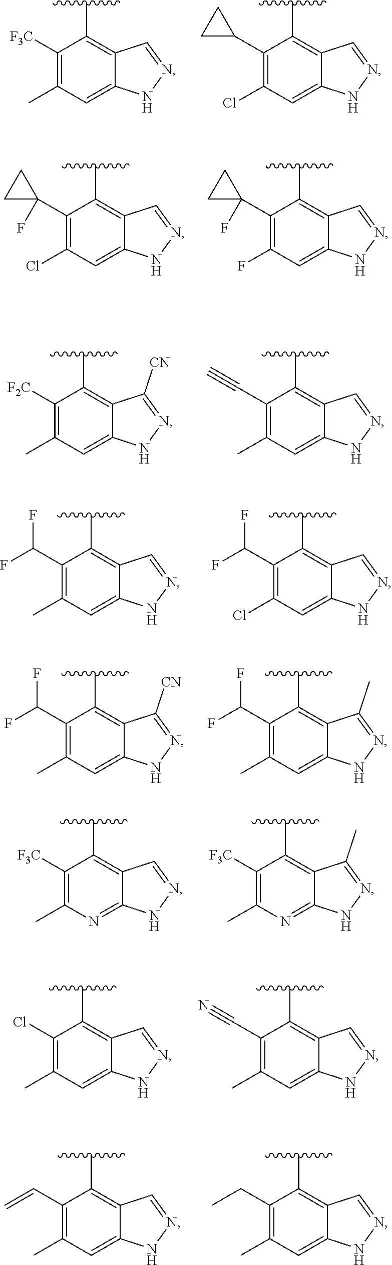

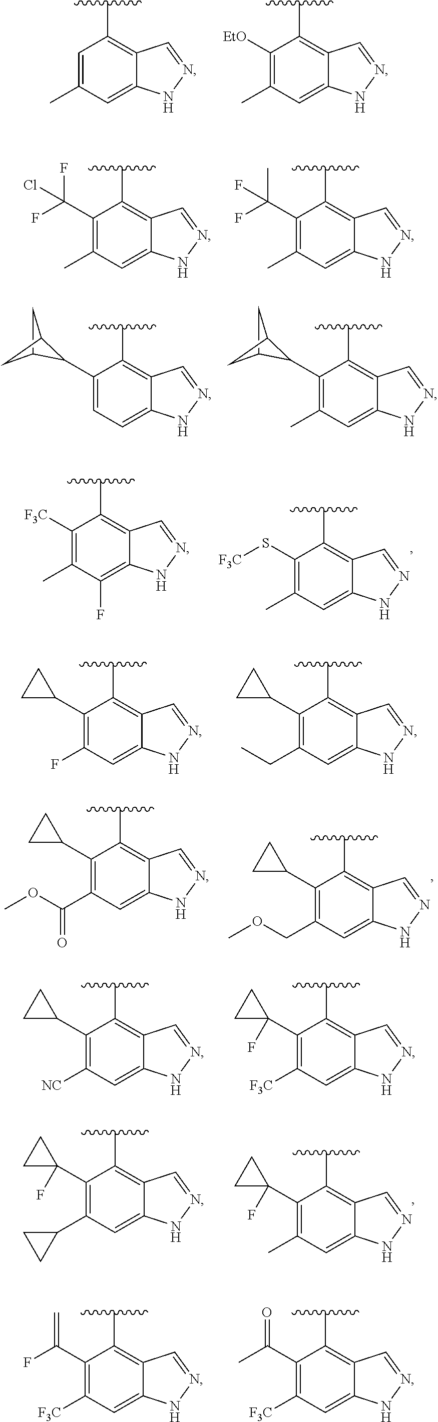

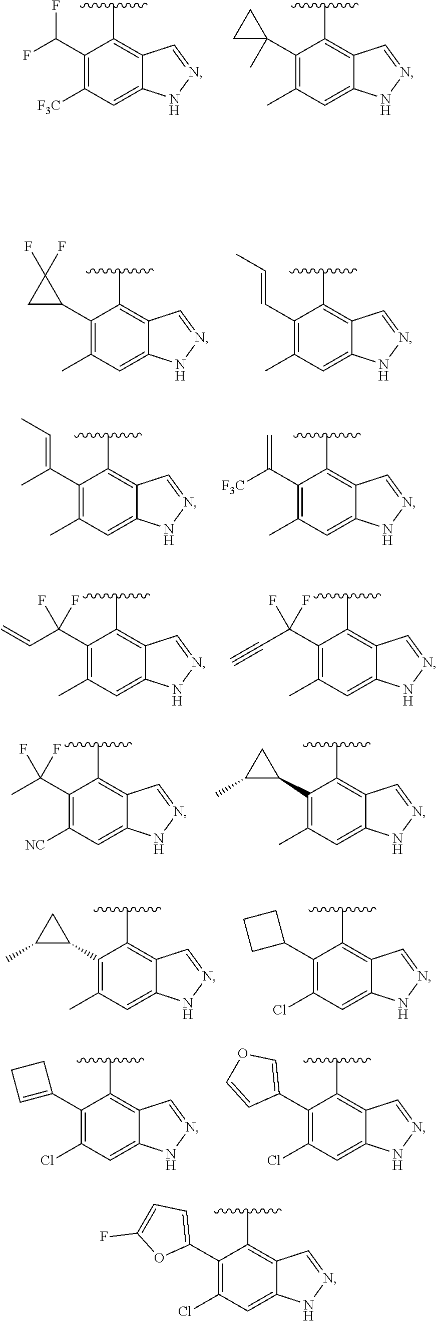

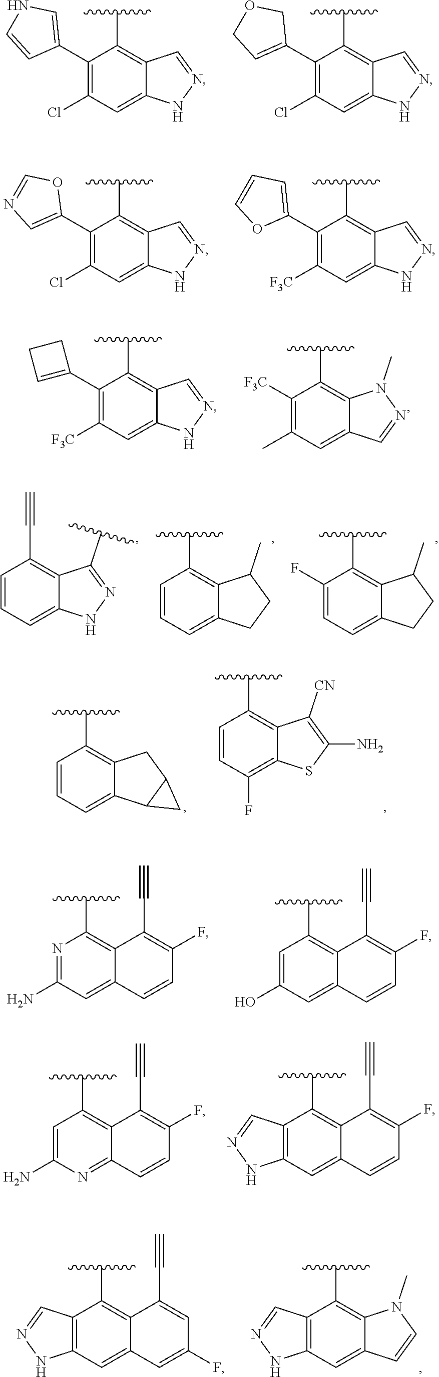

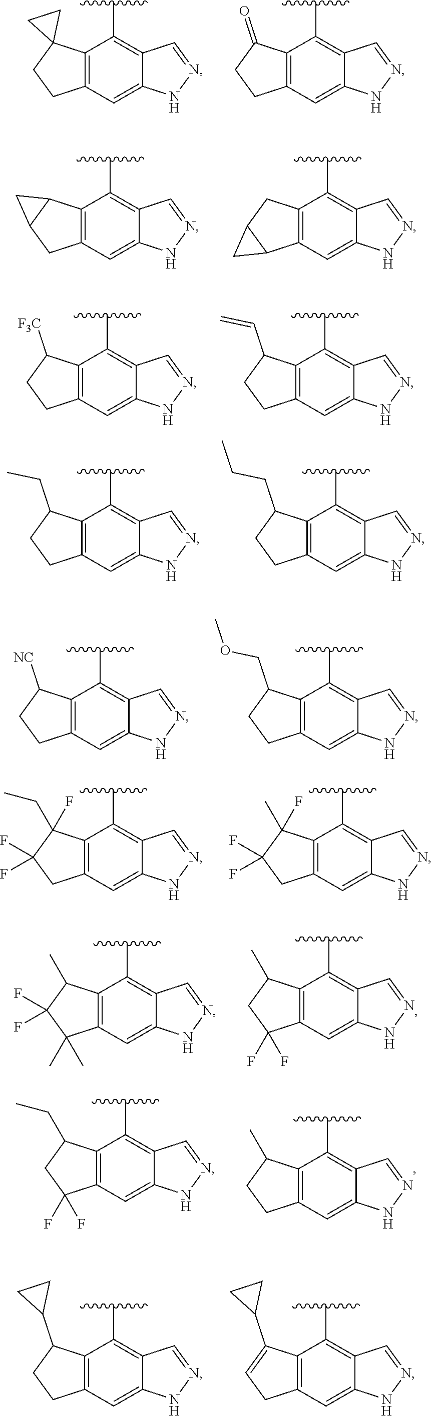

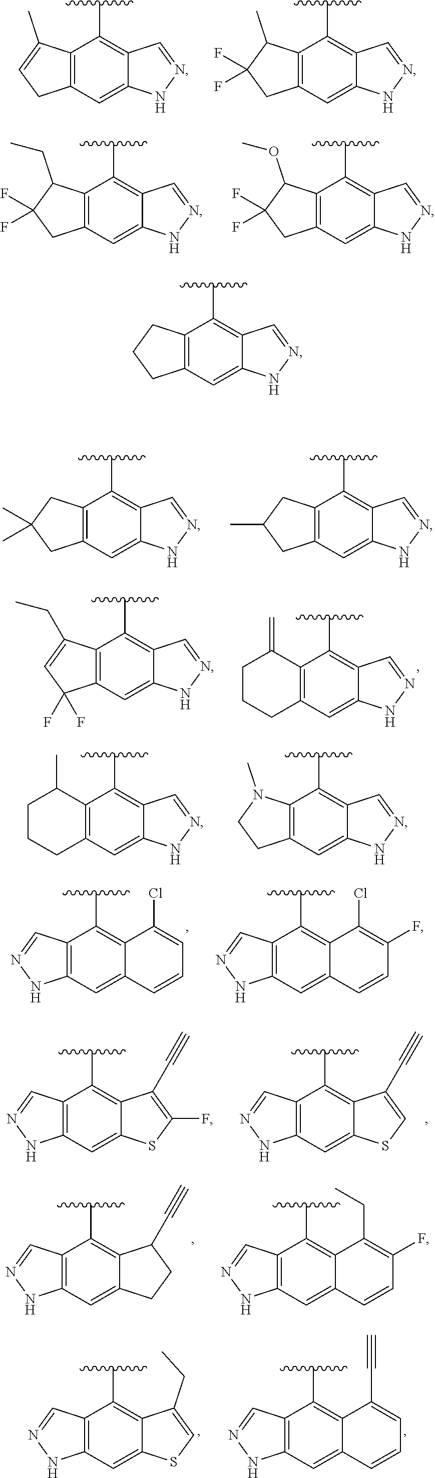

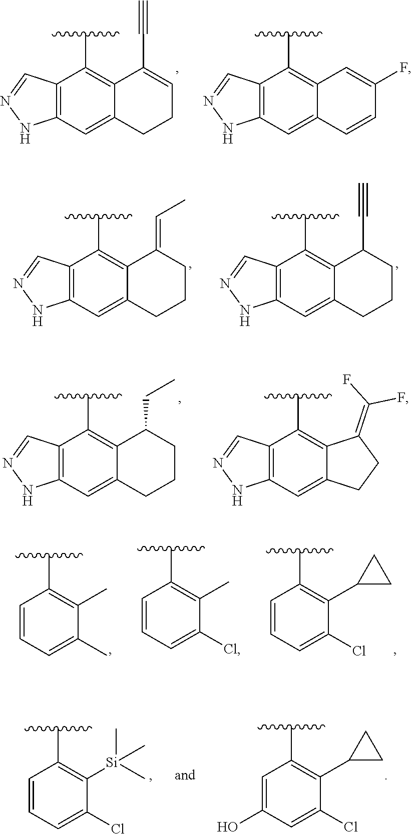

18. The compound of claim 1 or the pharmaceutically acceptable salt thereof, wherein
X^{A} is N,
X^{B} and X^{C} are independently selected from the group consisting of C(H) and (CR¹),
X^{D} is C, and
W^{B} is C.

19. The compound of claim 1 or the pharmaceutically acceptable salt thereof, wherein Ring Z is selected from the group consisting of:
(i) a 5- to 10-membered monocyclic, fused, bridged or spiro-bicyclic, or fused, bridged or spiro-tricyclic heterocycloalkyl, wherein the 5- to 10-membered monocyclic, fused, bridged or spiro-bicyclic, or fused, bridged or spiro-tricyclic heterocycloalkyl is saturated and contains 1 to 3 heteroatoms independently selected from the group consisting of N, S, and O, and wherein the 5- to 10-membered monocyclic, fused, bridged or spiro-bicyclic, or fused, bridged or spiro-tricyclic heterocycloalkyl is unsubstituted or substituted with 1 to 2 substituents R^{ZHC} selected from the group consisting of halo, hydroxy, C₁-C₃ alkyl, C₁-C₃ hydroxyalkyl, —C(H)(OH)CF₂H, —O—CH₂—O—(C₁-C₃ fluoroalkyl), C₂-C₄ fluoroalkenyl, and methylene (C₁-C₃ alkyl) (C₁-C₃ alkyl) carbamate;
(ii) 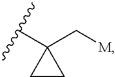
wherein M is selected from the group consisting of hydroxy, C₁-C₄ dialkylamino, and C₁-C₄ alkylamino, and wherein the cyclopropyl group is unsubstituted or independently substituted with up to 2 halo groups; 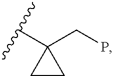
(iii) wherein P is a 5- to 10-membered monocyclic, fused bicyclic, bridged bicyclic, or spirocyclic heterocycloalkyl, wherein the 5- to 10-membered monocyclic, fused bicyclic, bridged bicyclic, or spirocyclic heterocycloalkyl is saturated and contains 1 to 2 heteroatoms selected from the group consisting of N, S, and O, wherein the 5- to 10-membered monocyclic, fused bicyclic, bridged bicyclic, or spirocyclic heterocycloalkyl is unsubstituted or substituted with 1 to 2 R^{P} substituents selected from the group consisting of halo, hydroxy, C₁-C₃ alkyl, C₁-C₃ hydroxyalkyl, C₁-C₃ cyanoalkyl, C₁-C₃ fluoroalkyl carbamoyl, C₁-C₃ alkoxy, C₁-C₃ fluoroalkoxy, cyano, C₃-C₆ cycloalkyl, C₃-C₆ fluorocycloalkyl, and —NHC(O)C₁-C₃alkyl, and wherein the cyclopropyl group is unsubstituted or independently substituted with up to 2 halo groups; and
(iv) a 4- to 8-membered monocyclic or fused, bridged or spiro-bicyclic cycloalkyl, wherein the 4- to 8-membered monocyclic or fused, bridged or spiro-bicyclic cycloalkyl is saturated and wherein the 4- to 8-membered monocyclic or fused, bridged or spiro-bicyclic cycloalkyl is unsubstituted or independently substituted with 1 to 3 substituents R^{ZC} selected from the group consisting of halo, hydroxy, C₁-C₃ alkyl, C₁-C₃ fluoroalkyl, C₁-C₃ hydroxyalkyl, C₁-C₃ hydroxyfluoroalkyl, C₃-C₄ cycloalkyl, C₃-C₄ fluorocycloalkyl, C₃-C₄ hydroxycycloalkyl, and C₃-C₄ hydroxyfluorocycloalkyl.

20.-22. (canceled)

23. The compound of claim 22 or the pharmaceutically acceptable salt thereof, wherein Ring Z is selected from the group consisting of:

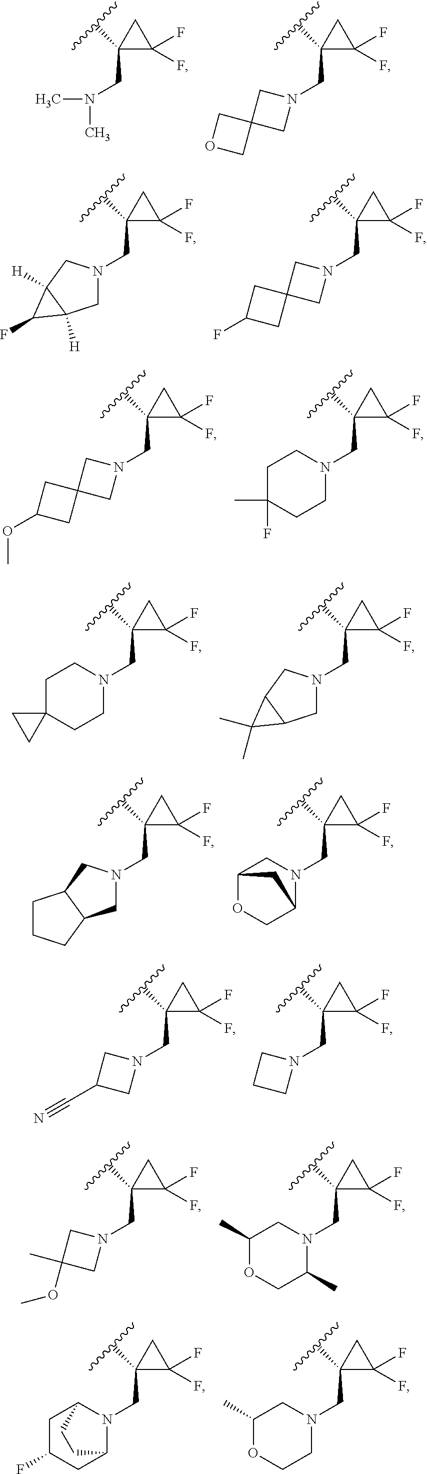

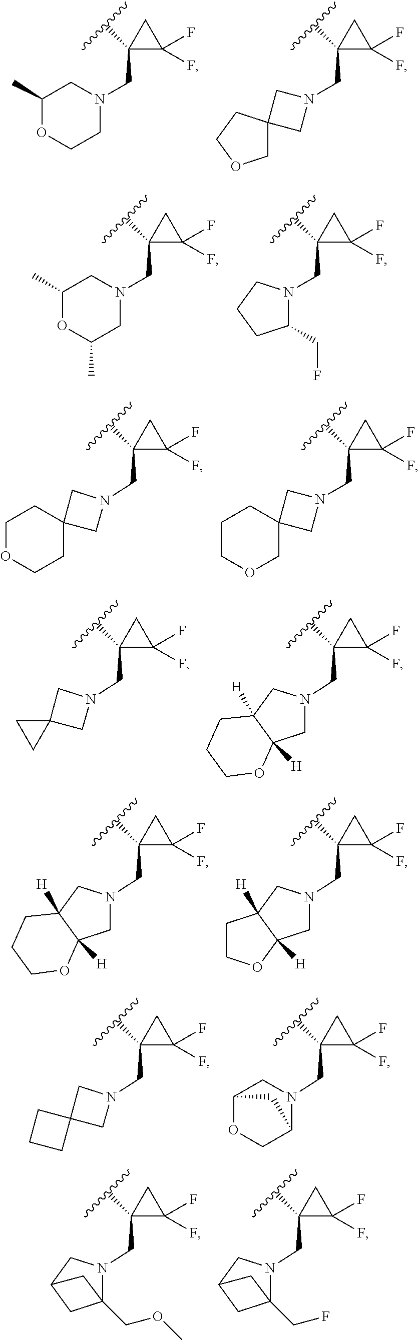

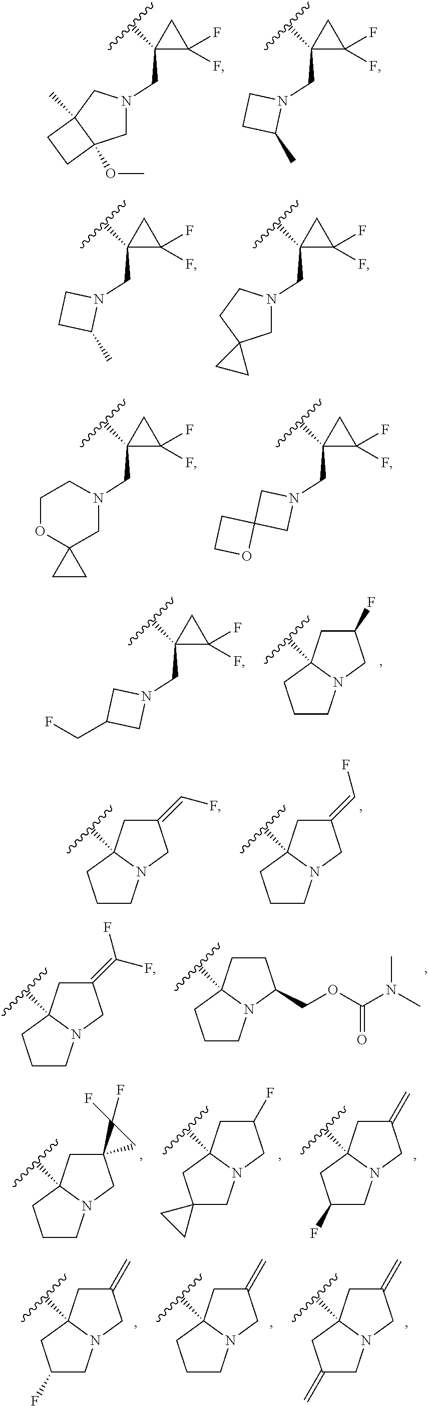

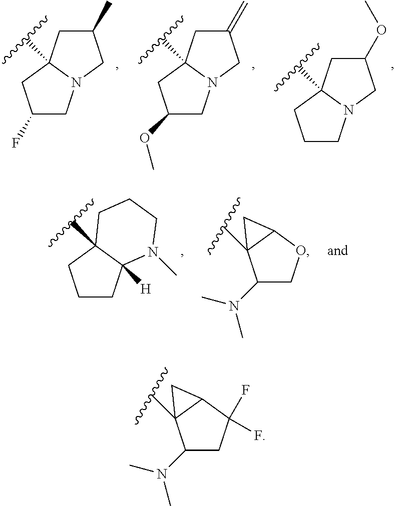

24. (canceled)

25. The compound of claim 1 or the pharmaceutically acceptable salt thereof, wherein the group 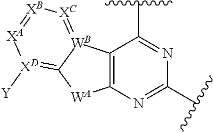
is selected from the group consisting of:

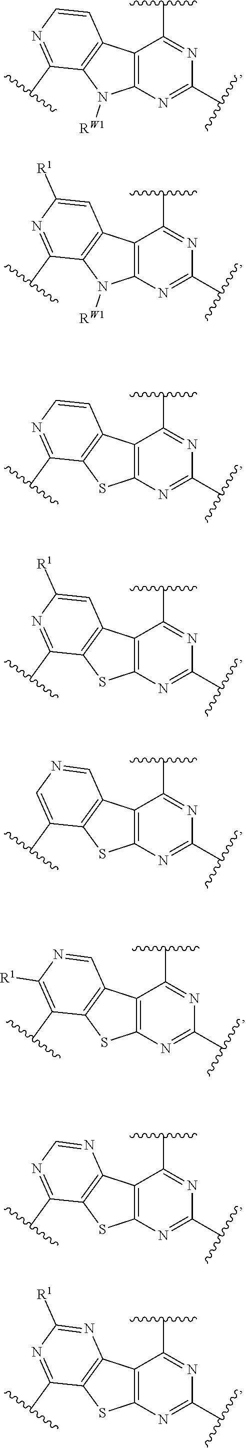

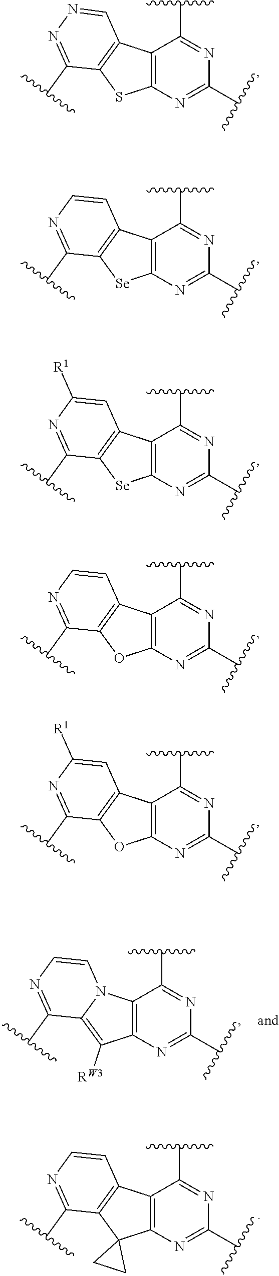

26. The compound of claim 1 or the pharmaceutically acceptable salt thereof, wherein the compound of Formula (I) has Formula (IA)

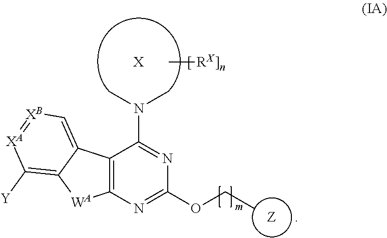

27. The compound of claim 1 or the pharmaceutically acceptable salt thereof, wherein the compound of Formula (I) has Formula (IB)

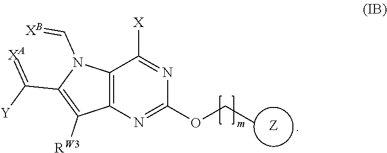

28. The compound of claim 1 selected from Examples 1-307 or the pharmaceutically acceptable salts thereof.

29. A compound of Formula (I) 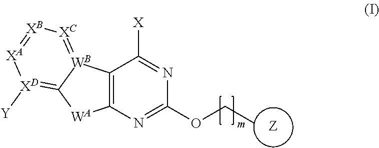
wherein:
X is: 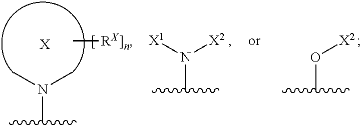
Ring X is selected from the group consisting of:
(i) a 5- to 9-membered monocyclic or fused bicyclic or bridged bicyclic heterocycloalkyl, wherein the 5- to 9-membered monocyclic or fused bicyclic or bridged bicyclic heterocycloalkyl is saturated and contains 0 to 2 heteroatom groups selected from the group consisting of N, S, S(O), S(O)₂ and O, in addition to the illustrated N atom; and
(ii) an 8- to 10-membered spiroheterocycloalkyl, wherein the 8- to 10-membered spiroheterocycloalkyl is saturated and contains 0 to 2 heteroatom groups selected from the group consisting of N, S, S(O), S(O)₂ and O, in addition to the illustrated N atom;
each R^{X} is independently selected from the group consisting of fluoro, cyano, hydroxy, oxo, C₁-C₆ alkyl, C₁-C₆ fluoroalkyl, C₁-C₆ alkoxy, C₁-C₆ fluoroalkoxy, C₁-C₆ cyanoalkyl, and C₁-C₆ hydroxyalkyl;
X¹ is selected from the group consisting of H, C₁-C₆ alkyl, C₁-C₆ fluoroalkyl, C₁-C₆ fluoroalkoxy, C₃-C₆ cycloalkyl, and C₃-C₆ fluorocycloalkyl;
X² is H or —(CR^{a}₂)ₚ—C^{X}, wherein C^{X} is:
(i) a 3- to 10-membered monocyclic or bicyclic cycloalkyl or an 8- to 10-membered spirocycloalkyl;
(ii) a 3- to 10-membered monocyclic or bicyclic heterocycloalkyl or an 8- to 10-membered spiroheterocycloalkyl, wherein the 3- to 10-membered monocyclic or bicyclic heterocycloalkyl or the 8- to 10-membered spiroheterocycloalkyl is saturated and contains 1 to 3 heteroatom groups independently selected from the group consisting of N, S, S(O), S(O)₂ and O;
wherein C^{X} is unsubstituted or substituted by 1 to 2 R^{C}^{^{X}} substituents independently selected from the group consisting of fluoro, hydroxy, oxo, cyano, C₁-C₆ alkyl, C₁-C₆ acyl, C₁-C₆ fluoroalkyl, C₁-C₆ alkoxy, C₁-C₆ fluoroalkoxy, C₁-C₆ hydroxyalkyl, C₁-C₆ cyanoalkyl, C₃-C₆ cycloalkyl, and C₃-C₆ fluorocycloalkyl;
each R^{a} is independently selected from the group consisting of hydrogen, halo, cyano, hydroxy, C₁-C₆ alkyl, C₁-C₆ fluoroalkyl, C₁-C₆ alkoxy, C₁-C₆ fluoroalkoxy, C₁-C₆ cyanoalkyl, C₁-C₆ fluorocyanoalkyl, C₃-C₆ cycloalkyl, and C₃-C₆ fluorocycloalkyl;
X^{A}, X^{B}, and X^{C}, are independently selected from the group consisting of N, C(H), and C(R¹);
X^{D} is selected from the group consisting of N and C;
each R¹ is independently selected from the group consisting of C₁-C₃ alkyl, C₁-C₃ fluoroalkyl, C₁-C₃ alkoxy, C₁-C₃ fluoroalkoxy, halo, hydroxy, oxo, cyano, C₁-C₃ alkylamino, C₁-C₃ dialkylamino, and C^{B};
wherein C^{B} is a monocyclic, saturated heterocycloalkyl containing 1 to 3 heteroatoms independently selected from the group consisting of N, S, and O;
wherein C^{B} is unsubstituted or substituted by 1 to 2 R^{C}^{^{B}} substituents independently selected from the group consisting of halo, hydroxy, oxo, cyano, C₁-C₃ alkyl, C₁-C₃ fluoroalkyl, C₁-C₃ alkoxy, C₁-C₃ fluoroalkoxy, C₃-C₆ cycloalkyl, and C₃-C₆ fluorocycloalkyl;
W^{A} is selected from the group consisting of N(R^{W1}), C(R^{W2})₂, O, S, and C(R^{W3});
R^{W1} is selected from the group consisting of C₁-C₃ alkyl, C₁-C₃ fluoroalkyl, C₃-C₆ cycloalkyl, and C₃-C₆ fluorocycloalkyl;
each R^{W2} is independently selected from the group consisting of fluoro, C₁-C₃ alkyl, C₁-C₃ fluoroalkyl, and hydroxy;
or alternatively, the two R^{W2}, together with the carbon atom to which they are attached form a C₃-C₆ cycloalkyl or C₃-C₆ fluorocycloalkyl;
R^{W3} is selected from the group consisting of H, halo, C₁-C₃ alkyl, C₁-C₃ alkoxy, C₁-C₃ fluoroalkyl, C₁-C₃ fluoroalkoxy, C₃-C₆ cycloalkyl, and C₃-C₆ fluorocycloalkyl;
W^{B} is C or N, wherein when:
W^{B} is C, then W^{A} is N(R^{W1}), C(R^{W2})₂, O, or S; and
W^{B} is N, then W^{A} is C(R^{W3});
Y is selected from the group consisting of C₁-C₆ alkyl, C₁-C₆ fluoroalkyl, and C^{Y};
C^{Y} is:
(i) an aryl selected from the group consisting of phenyl and naphthyl:
(ii) a 5- to 6-membered monocyclic heteroaryl, wherein the 5- to 6-membered monocyclic heteroaryl contains 1 to 3 heteroatoms independently selected from the group consisting of N, S, and O; or
(iii) a 9- to 10-membered bicyclic heteroaryl, wherein the 9- to 10-membered bicyclic heteroaryl contains 1 to 3 heteroatoms independently selected from the group consisting of N, O, and S; or
(iv) a 12- to 17-membered tri- or tetracyclic heterocycloalkyl, where at least 2 of the rings of the 12- to 17-membered tri- or tetracyclic heterocycloalkyl are aromatic, the third ring is partially unsaturated or aromatic, and the fourth ring, if present, is saturated, wherein the 12- to 17-membered tri- or tetracyclic heterocycloalkyl contains 1 to 4 heteroatoms independently selected from the group consisting of N, S, and O;
wherein C^{Y} is unsubstituted or substituted by 1 to 4 RY substituents independently selected from the group consisting of halo, hydroxy, cyano, C₁-C₆ alkyl, C₂-C₆ alkynyl, C₂-C₇ alkenyl, C₁-C₆ fluoroalkyl, C₁-C₆ alkoxy, C₁-C₆ haloalkoxy, C₂-C₆ fluoroalkynyl, C₂-C₇ fluoroalkenyl, C₁-C₆ alkylthio, C₁-C₆ fluoroalkylthio, amino, C₁-C₆ alkylamino, C₁-C₆ dialkylamino, tri (C₁-C₆ alkyl) silyl, cyano, C₁-C₆ cyanoalkyl, C₁-C₆ fluorocyanoalkyl, and R^{ya};
wherein R^{ya} is:
(a) a 3- to 9-membered monocyclic or bicyclic cycloalkyl;
(b) a 3- to 9-membered monocyclic or bicyclic cycloalkenyl;
(c) a 5- to 6-membered heteroaryl containing 1 to 3 heteroatoms independently selected from the group consisting of N, S, and O; or
(d) a 4- to 7-membered saturated heterocycloalkyl containing 1 to 2 heteroatoms independently selected from the group of N, S, and O;
wherein R^{ya} is unsubstituted or substituted by 1 to 2 substituents independently selected from the group consisting of halo, hydroxy, cyano, C₁-C₃ alkyl, C₁-C₃ fluoroalkyl, C₃-C₆ cycloalkyl, C₃-C₆ fluorocycloalkyl, C₁-C₃ alkoxy, C₁-C₃ fluoroalkoxy, and C₁-C₃ cyanoalkyl;
Ring Z is selected from the group consisting of:
(i) a 3- to 10-membered monocyclic, bicyclic, or tricyclic heterocycloalkyl, wherein the 3- to 10-membered monocyclic, bicyclic, or tricyclic heterocycloalkyl is saturated or partially unsaturated and contains 1 to 3 heteroatoms independently selected from the group consisting of N, S, and O; and
(ii) a 3- to 10-membered monocyclic, bicyclic, or tricyclic cycloalkyl, wherein the 3- to 10-membered monocyclic, bicyclic, or tricyclic cycloalkyl is saturated or partially unsaturated;
wherein ring Z is unsubstituted or independently substituted with 1 to 4 substituents R^{ZC} selected from the group consisting of halo, hydroxy, oxo, cyano, C₁-C₃ alkyl, C₁-C₃ fluoroalkyl, C₁-C₃ hydroxyalkyl, C₁-C₃ hydroxyfluoroalkyl, C₁-C₃ alkoxy, C₁-C₃ fluoroalkoxy, C₁-C₃ cyanoalkyl, C₃-C₆ cycloalkyl, C₃-C₆ fluorocycloalkyl, C₃-C₆ hydroxycycloalkyl, C₃-C₆ hydroxyfluorocycloalkyl, C₂-C₄ fluoroalkenyl, C₁-C₃ alkylamino, C₁-C₃ dialkylamino, methylene (C₁-C₃) alkyl)amino, methylenedi (C₁-C₃)alkylamino, and methylene (C₁-C₃ alkyl) (C₁-C₃ alkyl) carbamate;
wherein ring Z is optionally substituted with 1 P or —CH₂P, wherein P is a 4- to 10-membered monocyclic, bicyclic, or tricyclic heterocycloalkyl, wherein the 4- to 10-membered monocyclic, bicyclic, or tricyclic heterocycloalkyl is saturated or partially unsaturated and contains 1 to 2 heteroatoms selected from the group consisting of N, S, and O;
wherein P is unsubstituted or independently substituted with 1 to 4 R^{P} substituents selected from the group consisting of halo, hydroxy, oxo, cyano, C₁-C₃ alkyl, C₁-C₃ fluoroalkyl, C₁-C₃ hydroxyalkyl, C₁-C₃ hydroxyfluoroalkyl, C₁-C₃ alkoxy, C₁-C₃ fluoroalkoxy, C₁-C₃ cyanoalkyl, C₃-C₆ cycloalkyl, C₃-C₆ fluorocycloalkyl, C₃-C₆ hydroxycycloalkyl, C₃-C₆ hydroxyfluorocycloalkyl, carbamoyl, and —NHC(O)C₁-C₃alkyl;
subscript m is 0, 1, or 2;
subscript n is 0, 1, 2, or 3; and
subscript p is 0, 1, 2, or 3;
or a pharmaceutically acceptable salt thereof.

30. A pharmaceutical composition comprising the compound of claim 1 or the pharmaceutically acceptable salt thereof, and a pharmaceutically acceptable carrier.

31. A pharmaceutical composition comprising the compound of claim 1 or the pharmaceutically acceptable salt thereof, an additional anti-cancer agent, and a pharmaceutically acceptable carrier.

32.-35. (canceled)

36. A method of treating cancer comprising administering a therapeutically effective amount of the compound of claim 1, or the pharmaceutically acceptable salt thereof, to a subject in need of such treatment.

37. The method of claim 36, further comprising administering an additional active agent to the subject.

38.-44. (canceled)
